(19) United States
(12) Patent Application Publication
(10) Pub. No.: US 2024/0090329 A1
(43) Pub. Date: Mar. 14, 2024

(54) COMPOUND, ORGANIC ELECTROLUMINESCENT ELEMENT AND ELECTRONIC DEVICE

(71) Applicant: IDEMITSU KOSAN CO.,LTD., Tokyo (JP)

(72) Inventors: Hiroaki ITOI, Tokyo (JP); Yuki NAKANO, Tokyo (JP); Taro YAMAKI, Tokyo (JP); Maiko IIDA, Tokyo (JP); Takamoto MORITA, Tokyo (JP); Shintaro BAN, Tokyo (JP); Ryota TAKAHASHI, Tokyo (JP); Yu KUDO, Tokyo (JP); Yoshinao SHIRASAKI, Tokyo (JP)

(73) Assignee: IDEMITSU KOSAN CO.,LTD., Tokyo (JP)

(21) Appl. No.: 18/032,821

(22) PCT Filed: Oct. 1, 2021
(86) PCT No.: PCT/JP2021/036397

§ 371 Date: Apr. 20, 2023

(30) Foreign Application Priority Data
Oct. 27, 2020 (JP) 2020-179704
Feb. 19, 2021 (JP) 2021-025304
Mar. 31, 2021 (JP) 2021-062404
Sep. 7, 2021 (JP) 2021-145819

(51) Int. Cl. H10K 85/60; C07D 311/78; C09K 11/06

(52) U.S. Cl. CPC H10K 85/6574; C07D 311/78; H10K 85/622; C09K 11/06; H10K 50/11

(57) ABSTRACT

A compound includes: at least one group represented by a formula (11) below; and a single benz[de]anthracene derivative skeleton represented by a formula (1000) below in a molecule, in which Ar₁ is a substituted or unsubstituted aryl group including at least four rings, at least one of R₁₀ to R₁₉ is a group represented by the formula (11), L₁ is a substituted or unsubstituted arylene group having 6 to 15 ring carbon atoms or a substituted or unsubstituted divalent heterocyclic group having 5 to 15 ring atoms, and mx is 1, 2, or 3.

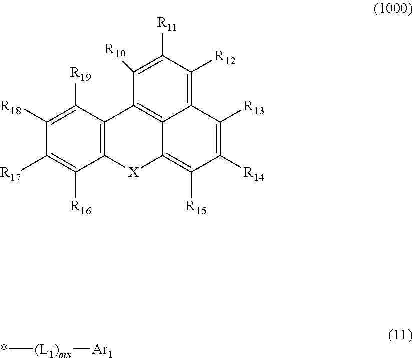

### TECHNICAL FIELD

The present invention relates to a compound, an organic electroluminescence device, and an electronic device.

### BACKGROUND ART

An organic electroluminescence device (hereinafter, occasionally referred to as “organic EL device”) has found its application in a full-color display for mobile phones, televisions and the like. When a voltage is applied to an organic EL device, holes are injected from an anode and electrons are injected from a cathode into an emitting layer. The injected electrons and holes are recombined in the emitting layer to form excitons. Specifically, according to the electron spin statistics theory, singlet excitons and triplet excitons are generated at a ratio of 25%:75%.

Various studies have been made on a compound to be used for an organic EL device in order to enhance the performance of the organic EL device (see, for instance, Patent Literatures 1 to 4). The performance of the organic EL device is evaluable in terms of, for instance, luminance, emission wavelength, chromaticity, luminous efficiency, drive voltage, and lifetime.

### CITATION LIST

### Patent Literature(s)

Patent Literature 1 US Patent Application Publication No. 2015/270498
Patent Literature 2 US Patent Application Publication No. 2015/001479
Patent Literature 3 US Patent Application Publication No. 2015/069344
Patent Literature 4 US Patent Application Publication No. 2017/331051

### SUMMARY OF THE INVENTION

### Problem(s) to be Solved by the Invention

An object of the invention is to provide a compound capable of improving performance of an organic EL device, an organic electroluminescence device containing the compound, and an electronic device including the organic electroluminescence device.

Another object of the invention is to provide a compound capable of providing an organic electroluminescence device, in which a plurality of emitting layers are layered, in a favorable balance between a luminous efficiency and a lifetime when the compound is used in an emitting layer close to an anode of the organic electroluminescence device, and to provide an electronic device including the organic electroluminescence device.

### Means for Solving the Problem(s)

According to an aspect of the invention, there is provided a compound having at least one group represented by a formula (11) below and a single benz[de]anthracene derivative skeleton represented by a formula (1000) below in a molecule.

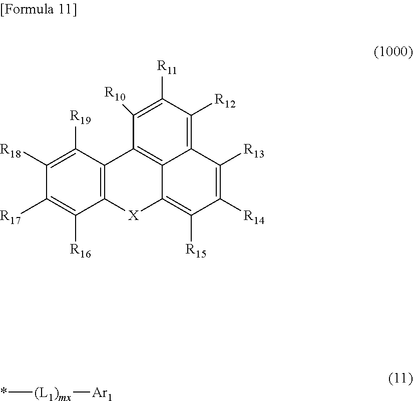

In the formula (1000):
X is an oxygen atom, a sulfur atom, C(R₂₀₀₁)(R₂₀₀₂), or Si(R₂₀₀₃)(R₂₀₀₄);
R₂₀₀₁ to R₂₀₀₄ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
at least one combination of adjacent two or more of R₁₀ to R₁₉ are mutually bonded to form a substituted or unsubstituted monocyclic ring, mutually bonded to form a substituted or unsubstituted fused ring, or not mutually bonded;
R₁₀ to R₁₉ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a nitro group, a substituted or unsubstituted aryl group having 6 to 50 ring carbon atoms, a substituted or unsubstituted heterocyclic group having 5 to 50 ring atoms, or a group represented by the formula (11), and
at least one of R₁₀ to R₁₉ is a group represented by the formula (11);
when a plurality of groups represented by the formula (11) are present, the plurality of groups represented by the formula (11) are mutually the same or different;
L₁ is a substituted or unsubstituted arylene group having 6 to 15 ring carbon atoms, or a substituted or unsubstituted divalent heterocyclic group having 5 to 15 ring atoms;
mx is 1, 2, or 3;
when two or more L₁ are present, the two or more L₁ are mutually the same or different;
Ar₁ is a substituted or unsubstituted aryl group including four or more rings;
when two or more Ar₁ are present, the two or more Ar₁ are mutually the same or different;
* in the formula (11) represents a bonding position;
in a compound represented by the formula (1000), R₉₀₁, R₉₀₂, R₉₀₃, R₉₀₄, R₉₀₅, R₉₀₅, R₉₀₇, R₈₀, and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₅ are present, the plurality of R₉₀₅ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

According to another aspect of the invention, there is provided a compound having at least one group represented by a formula (110A) below and a single benz[de]anthracene derivative skeleton represented by a formula (1000A) below in a molecule.

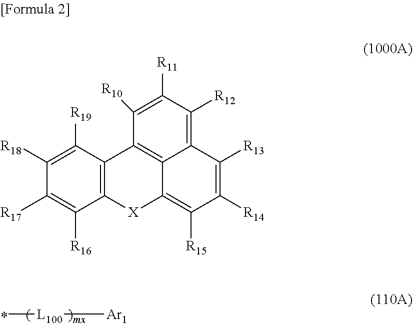

In the formula (1000A):
X is an oxygen atom, a sulfur atom, C(R₂₀₀₁)(R₂₀₀₂), or Si(R₂₀₀₃)(R₂₀₀₄);
R₂₀₀₁ to R₂₀₀₄ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
at least one combination of adjacent two or more of R₁₀ to R₁₉ are mutually bonded to form a substituted or unsubstituted monocyclic ring, mutually bonded to form a substituted or unsubstituted fused ring, or not mutually bonded;
R₁₀ to R₁₉ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a nitro group, a substituted or unsubstituted aryl group having 6 to 50 ring carbon atoms, a substituted or unsubstituted heterocyclic group having 5 to 50 ring atoms, or a group represented by the formula (110A);
at least one of R₁₃ or R₁₈ is a group represented by the formula (110A);
when a plurality of groups represented by the formula (110A) are present, the plurality of groups represented by the formula (110A) are mutually the same or different;
L₁₀₀ is a single bond, a substituted or unsubstituted arylene group having 6 to 50 ring carbon atoms, or a substituted or unsubstituted divalent heterocyclic group having 5 to 50 ring atoms;
mx is 1, 2, or 3;
when two or more L₁₀₀ are present, the two or more L₁₀₀ are mutually the same or different;
Ar₁ is a substituted or unsubstituted aryl group including four or more rings;
when two or more Ar₁ are present, the two or more Ar₁ are mutually the same or different;
* in the formula (110A) represents a bonding position;
in a compound represented by the formula (1000A), R₉₀₁, R₉₀₂, R₉₀₃, R₉₀₄, R₉₀₅, R₉₀₆, R₉₀₇, R₈₀₁, and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₅ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

According to still another aspect of the invention, there is provided an organic electroluminescence device including: a cathode, an anode, and at least one organic layer provided between the cathode and the anode, in which the at least one organic layer includes an emitting layer and at least one of the at least one organic layer contains the compound according to the above aspect of the invention.

According to a further aspect of the invention, there is provided an organic electroluminescence device including: a cathode, an anode, and at least one emitting layer provided between the cathode and the anode, in which the at least one emitting layer includes a first emitting layer and a second emitting layer, the first emitting layer contains a first compound, and the first compound is a compound represented by a formula (1000B) below and having at least one group represented by a formula (110) below.

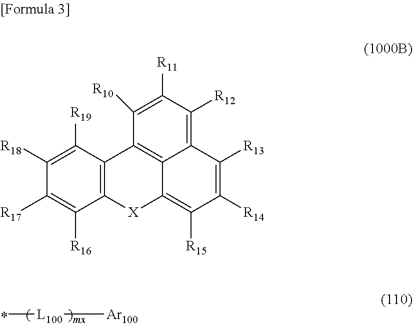

In the formula (1000B):
X is an oxygen atom, a sulfur atom, C(R₂₀₀₁)(R₂₀₀₂), or Si(R₂₀₀₃)(R₂₀₀₄);
R₂₀₀₁ to R₂₀₀₄ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
at least one combination of adjacent two or more of R₁₀ to R₁₉ are mutually bonded to form a substituted or unsubstituted monocyclic ring, mutually bonded to form a substituted or unsubstituted fused ring, or not mutually bonded;
R₁₀ to R₁₉ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a nitro group, a substituted or unsubstituted aryl group having 6 to 50 ring carbon atoms, a substituted or unsubstituted heterocyclic group having 5 to 50 ring atoms, or a group represented by the formula (110);
at least one of R₁₀ to R₁₉ is a group represented by the formula (110);
when a plurality of groups represented by the formula (110) are present, the plurality of groups represented by the formula (110) are mutually the same or different;
L₁₀₀ is a single bond, a substituted or unsubstituted arylene group having 6 to 50 ring carbon atoms, or a substituted or unsubstituted divalent heterocyclic group having 5 to 50 ring atoms;
mx is 1, 2, or 3;
when two or more L₁₀₀ are present, the two or more L₁₀₀ are mutually the same or different;
Ar₁₀₀ is a substituted or unsubstituted aryl group including three or more rings or a substituted or unsubstituted heterocyclic group including two or more aromatic rings and one or more heterocyclic rings;
Ar₁₀₀ does not include an anthracene ring;
when two or more Ar₁₀₀ are present, the two or more Ar₁₀₀ are mutually the same or different; and
* in the formula (110) represents a bonding position;
in the first compound represented by the formula (1000B), R₉₀₁, R₉₀₂, R₉₀₃, R₉₀₄, R₉₀₅, R₉₀₆, R₉₀₇, R₈₀₁, and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₅ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

According to a still further aspect of the invention, there is provided an electronic device including the organic electroluminescence device according to the above aspect of the invention.

According to the above aspects of the invention, there can be provided a compound capable of improving performance of an organic EL device, an organic electroluminescence device containing the compound, and an electronic device including the organic electroluminescence device.

Moreover, according to the above aspects of the invention, there can be provided a compound capable of providing an organic electroluminescence device, in which a plurality of emitting layers are layered, in a favorable balance between a luminous efficiency and a lifetime when the compound is used in an emitting layer close to an anode of the organic electroluminescence device, and to provide an electronic device including the organic electroluminescence device.

### BRIEF DESCRIPTION OF DRAWING(S)

FIG. 1 schematically shows an exemplary arrangement of an organic electroluminescence device according to an exemplary embodiment of the invention.

### DESCRIPTION OF EMBODIMENT(S)

### Definitions

Herein, a hydrogen atom includes isotopes having different numbers of neutrons, specifically, protium, deuterium and tritium.

In chemical formulae herein, it is assumed that a hydrogen atom (i.e. protium, deuterium and tritium) is bonded to each of bondable positions that are not annexed with signs “R” or the like or “D” representing a deuterium.

Herein, the ring carbon atoms refer to the number of carbon atoms among atoms forming a ring of a compound (e.g., a monocyclic compound, fused-ring compound, crosslinking compound, carbon ring compound, and heterocyclic compound) in which the atoms are bonded with each other to form the ring. When the ring is substituted by a substituent(s), carbon atom(s) contained in the substituent(s) is not counted in the ring carbon atoms. Unless otherwise specified, the same applies to the “ring carbon atoms” described later. For instance, a benzene ring has 6 ring carbon atoms, a naphthalene ring has 10 ring carbon atoms, a pyridine ring has 5 ring carbon atoms, and a furan ring has 4 ring carbon atoms. Further, for instance, 9,9-diphenylfluorenyl group has 13 ring carbon atoms and 9,9′-spirobifluorenyl group has 25 ring carbon atoms.

When a benzene ring is substituted by a substituent in a form of, for instance, an alkyl group, the number of carbon atoms of the alkyl group is not counted in the number of the ring carbon atoms of the benzene ring. Accordingly, the benzene ring substituted by an alkyl group has 6 ring carbon atoms. When a naphthalene ring is substituted by a substituent in a form of, for instance, an alkyl group, the number of carbon atoms of the alkyl group is not counted in the number of the ring carbon atoms of the naphthalene ring. Accordingly, the naphthalene ring substituted by an alkyl group has 10 ring carbon atoms.

Herein, the ring atoms refer to the number of atoms forming a ring of a compound (e.g., a monocyclic compound, fused-ring compound, crosslinking compound, carbon ring compound, and heterocyclic compound) in which the atoms are bonded to each other to form the ring (e.g., monocyclic ring, fused ring, and ring assembly). Atom(s) not forming the ring (e.g., hydrogen atom(s) for saturating the valence of the atom which forms the ring) and atom(s) in a substituent by which the ring is substituted are not counted as the ring atoms. Unless otherwise specified, the same applies to the “ring atoms” described later. For instance, a pyridine ring has 6 ring atoms, a quinazoline ring has 10 ring atoms, and a furan ring has 5 ring atoms. For instance, the number of hydrogen atom(s) bonded to a pyridine ring or the number of atoms forming a substituent are not counted as the pyridine ring atoms. Accordingly, a pyridine ring bonded with a hydrogen atom(s) or a substituent(s) has 6 ring atoms. For instance, the hydrogen atom(s) bonded to carbon atom(s) of a quinazoline ring or the atoms forming a substituent are not counted as the quinazoline ring atoms. Accordingly, a quinazoline ring bonded with hydrogen atom(s) or a substituent(s) has 10 ring atoms.

Herein, “XX to YY carbon atoms” in the description of “substituted or unsubstituted ZZ group having XX to YY carbon atoms” represent carbon atoms of an unsubstituted ZZ group and do not include carbon atoms of a substituent(s) of the substituted ZZ group. Herein, “YY” is larger than “XX,” “XX” representing an integer of 1 or more and “YY” representing an integer of 2 or more.

Herein, “XX to YY atoms” in the description of “substituted or unsubstituted ZZ group having XX to YY atoms” represent atoms of an unsubstituted ZZ group and do not include atoms of a substituent(s) of the substituted ZZ group. Herein, “YY” is larger than “XX,” “XX” representing an integer of 1 or more and “YY” representing an integer of 2 or more.

Herein, an unsubstituted ZZ group refers to an “unsubstituted ZZ group” in a “substituted or unsubstituted ZZ group,” and a substituted ZZ group refers to a “substituted ZZ group” in a “substituted or unsubstituted ZZ group.”

Herein, the term “unsubstituted” used in a “substituted or unsubstituted ZZ group” means that a hydrogen atom(s) in the ZZ group is not substituted with a substituent(s). The hydrogen atom(s) in the “unsubstituted ZZ group” is protium, deuterium, or tritium.

Herein, the term “substituted” used in a “substituted or unsubstituted ZZ group” means that at least one hydrogen atom in the ZZ group is substituted with a substituent. Similarly, the term “substituted” used in a “BB group substituted by AA group” means that at least one hydrogen atom in the BB group is substituted with the AA group.

Substituents Mentioned Herein

Substituents mentioned herein will be described below.

An “unsubstituted aryl group” mentioned herein has, unless otherwise specified herein, 6 to 50, preferably 6 to 30, more preferably 6 to 18 ring carbon atoms.

An “unsubstituted heterocyclic group” mentioned herein has, unless otherwise specified herein, 5 to 50 ring atoms, preferably 5 to 30 ring atoms, more preferably 5 to 18 ring atoms.

An “unsubstituted alkyl group” mentioned herein has, unless otherwise specified herein, 1 to 50 carbon atoms, preferably 1 to 20 carbon atoms, more preferably 1 to 6 carbon atoms.

An “unsubstituted alkenyl group” mentioned herein has, unless otherwise specified herein, 2 to 50, preferably 2 to 20, more preferably 2 to 6 carbon atoms.

An “unsubstituted alkynyl group” mentioned herein has, unless otherwise specified herein, 2 to 50 carbon atoms, preferably 2 to 20 carbon atoms, more preferably 2 to 6 carbon atoms.

An “unsubstituted cycloalkyl group” mentioned herein has, unless otherwise specified herein, 3 to 50, preferably 3 to 20, more preferably 3 to 6 ring carbon atoms.

An “unsubstituted arylene group” mentioned herein has, unless otherwise specified herein, 6 to 50 ring carbon atoms, preferably 6 to 30 ring carbon atoms, more preferably 6 to 18 ring carbon atoms.

An “unsubstituted divalent heterocyclic group” mentioned herein has, unless otherwise specified herein, 5 to 50 ring atoms, preferably 5 to 30 ring atoms, more preferably 5 to 18 ring atoms.

An “unsubstituted alkylene group” mentioned herein has, unless otherwise specified herein, 1 to 50 carbon atoms, preferably 1 to 20 carbon atoms, more preferably 1 to 6 carbon atoms.

Substituted or Unsubstituted Aryl Group

Specific examples (specific example group G1) of the “substituted or unsubstituted aryl group” mentioned herein include unsubstituted aryl groups (specific example group G1A) below and substituted aryl groups (specific example group G1B). Herein, an unsubstituted aryl group refers to an “unsubstituted aryl group” in a “substituted or unsubstituted aryl group,” and a substituted aryl group refers to a “substituted aryl group” in a “substituted or unsubstituted aryl group.” A simply termed “aryl group” herein includes both of an “unsubstituted aryl group” and a “substituted aryl group.”

The “substituted aryl group” refers to a group derived by substituting at least one hydrogen atom in an “unsubstituted aryl group” with a substituent. Examples of the “substituted aryl group” include a group derived by substituting at least one hydrogen atom in the “unsubstituted aryl group” in the specific example group G1A below with a substituent, and examples of the substituted aryl group in the specific example group G1B below. It should be noted that the examples of the “unsubstituted aryl group” and the “substituted aryl group” mentioned herein are merely exemplary, and the “substituted aryl group” mentioned herein includes a group derived by further substituting a hydrogen atom bonded to a carbon atom of a skeleton of a “substituted aryl group” in the specific example group G1B below, and a group derived by further substituting a hydrogen atom of a substituent of the “substituted aryl group” in the specific example group G1B below.

Unsubstituted Aryl Group (Specific Example Group G1A): phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, benzanthryl group, phenanthryl group, benzophenanthryl group, phenalenyl group, pyrenyl group, chrysenyl group, benzochrysenyl group, triphenylenyl group, benzotriphenylenyl group, tetracenyl group, pentacenyl group, fluorenyl group, 9,9′-spirobifluorenyl group, benzofluorenyl group, dibenzofluorenyl group, fluoranthenyl group, benzofluoranthenyl group, perylenyl group, and a monovalent aryl group derived by removing one hydrogen atom from cyclic structures represented by formulae (TEMP-1) to (TEMP-15) below.

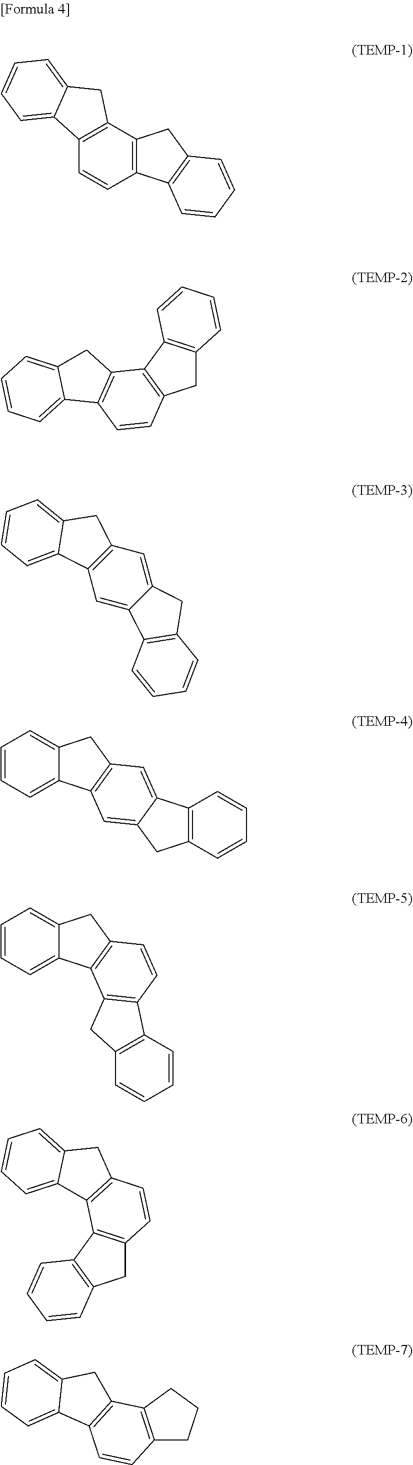

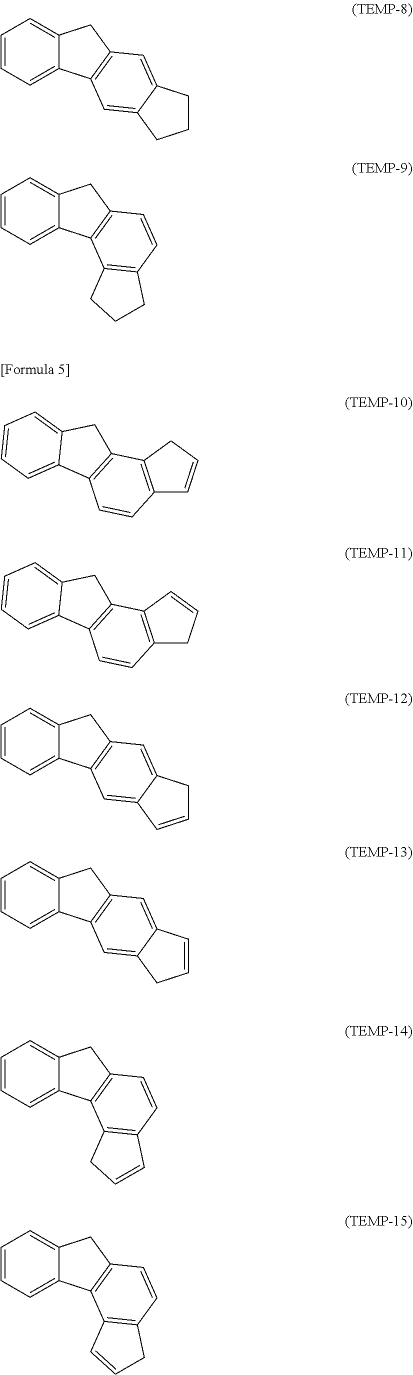

Substituted Aryl Group (Specific Example Group G1B):

o-tolyl group, m-tolyl group, p-tolyl group, para-xylyl group, meta-xylyl group, ortho-xylyl group, para-isopropylphenyl group, meta-isopropylphenyl group, ortho-isopropylphenyl group, para-t-butylphenyl group, meta-t-butylphenyl group, ortho-t-butylphenyl group, 3,4,5-trimethylphenyl group, 9,9-dimethylfluorenyl group, 9,9-diphenylfluorenyl group, 9,9-bis(4-methylphenyl)fluorenyl group, 9,9-bis(4-isopropylphenyl)fluorenyl group, 9,9-bis(4-t-butylphenyl)fluorenyl group, cyanophenyl group, triphenylsilylphenyl group, trimethylsilylphenyl group, phenylnaphthyl group, naphthylphenyl group, and a group derived by substituting at least one hydrogen atom of a monovalent group derived from the cyclic structures represented by the formulae (TEMP-1) to (TEMP-15) with a substituent.

Substituted or Unsubstituted Heterocyclic Group

The “heterocyclic group” mentioned herein refers to a cyclic group having at least one hetero atom in the ring atoms. Specific examples of the hetero atom include a nitrogen atom, oxygen atom, sulfur atom, silicon atom, phosphorus atom, and boron atom.

The “heterocyclic group” mentioned herein is a monocyclic group or a fused-ring group.

The “heterocyclic group” mentioned herein is an aromatic heterocyclic group or a non-aromatic heterocyclic group.

Specific examples (specific example group G2) of the “substituted or unsubstituted heterocyclic group” mentioned herein include unsubstituted heterocyclic groups (specific example group G2A) and substituted heterocyclic groups (specific example group G2B). Herein, an unsubstituted heterocyclic group refers to an “unsubstituted heterocyclic group” in a “substituted or unsubstituted heterocyclic group,” and a substituted heterocyclic group refers to a “substituted heterocyclic group” in a “substituted or unsubstituted heterocyclic group.” A simply termed “heterocyclic group” herein includes both of an “unsubstituted heterocyclic group” and a “substituted heterocyclic group.”

The “substituted heterocyclic group” refers to a group derived by substituting at least one hydrogen atom in an “unsubstituted heterocyclic group” with a substituent. Specific examples of the “substituted heterocyclic group” include a group derived by substituting at least one hydrogen atom in the “unsubstituted heterocyclic group” in the specific example group G2A below with a substituent, and examples of the substituted heterocyclic group in the specific example group G2B below. It should be noted that the examples of the “unsubstituted heterocyclic group” and the “substituted heterocyclic group” mentioned herein are merely exemplary, and the “substituted heterocyclic group” mentioned herein includes a group derived by further substituting a hydrogen atom bonded to a ring atom of a skeleton of a “substituted heterocyclic group” in the specific example group G2B below, and a group derived by further substituting a hydrogen atom of a substituent of the “substituted heterocyclic group” in the specific example group G2B below.

The specific example group G2A includes, for instance, unsubstituted heterocyclic groups including a nitrogen atom (specific example group G2A1) below, unsubstituted heterocyclic groups including an oxygen atom (specific example group G2A2) below, unsubstituted heterocyclic groups including a sulfur atom (specific example group G2A3) below, and monovalent heterocyclic groups (specific example group G2A4) derived by removing a hydrogen atom from cyclic structures represented by formulae (TEMP-16) to (TEMP-33) below.

The specific example group G2B includes, for instance, substituted heterocyclic groups including a nitrogen atom (specific example group G2B1) below, substituted heterocyclic groups including an oxygen atom (specific example group G2B2) below, substituted heterocyclic groups including a sulfur atom (specific example group G2B3) below, and groups derived by substituting at least one hydrogen atom of the monovalent heterocyclic groups (specific example group G2B4) derived from the cyclic structures represented by formulae (TEMP-16) to (TEMP-33) below.

Unsubstituted Heterocyclic Groups Including Nitrogen Atom (Specific Example Group G2A1):
pyrrolyl group, imidazolyl group, pyrazolyl group, triazolyl group, tetrazolyl group, oxazolyl group, isoxazolyl group, oxadiazolyl group, thiazolyl group, isothiazolyl group, thiadiazolyl group, pyridyl group, pyridazynyl group, pyrimidinyl group, pyrazinyl group, triazinyl group, indolyl group, isoindolyl group, indolizinyl group, quinolizinyl group, quinolyl group, isoquinolyl group, cinnolyl group, phthalazinyl group, quinazolinyl group, quinoxalinyl group, benzimidazolyl group, indazolyl group, phenanthrolinyl group, phenanthridinyl group, acridinyl group, phenazinyl group, carbazolyl group, benzocarbazolyl group, morpholino group, phenoxazinyl group, phenothiazinyl group, azacarbazolyl group, and diazacarbazolyl group.

Unsubstituted Heterocyclic Groups Including Oxygen Atom (Specific Example Group G2A2):
furyl group, oxazolyl group, isoxazolyl group, oxadiazolyl group, xanthenyl group, benzofuranyl group, isobenzofuranyl group, dibenzofuranyl group, naphthobenzofuranyl group, benzoxazolyl group, benzisoxazolyl group, phenoxazinyl group, morpholino group, dinaphthofuranyl group, azadibenzofuranyl group, diazadibenzofuranyl group, azanaphthobenzofuranyl group, and diazanaphthobenzofuranyl group.

Unsubstituted Heterocyclic Groups Including Sulfur Atom (Specific Example Group G2A3):
thienyl group, thiazolyl group, isothiazolyl group, thiadiazolyl group, benzothiophenyl group (benzothienyl group), isobenzothiophenyl group (isobenzothienyl group), dibenzothiophenyl group (dibenzothienyl group), naphthobenzothiophenyl group (nahthobenzothienyl group), benzothiazolyl group, benzisothiazolyl group, phenothiazinyl group, dinaphthothiophenyl group (dinaphthothienyl group), azadibenzothiophenyl group (azadibenzothienyl group), diazadibenzothiophenyl group (diazadibenzothienyl group), azanaphthobenzothiophenyl group (azanaphthobenzothienyl group), and diazanaphthobenzothiophenyl group (diazanaphthobenzothienyl group).

Monovalent Heterocyclic Groups Derived by Removing One Hydrogen Atom from Cyclic Structures Represented by Formulae (TEMP-16) to (TEMP-33) (Specific Example Group G2A4):

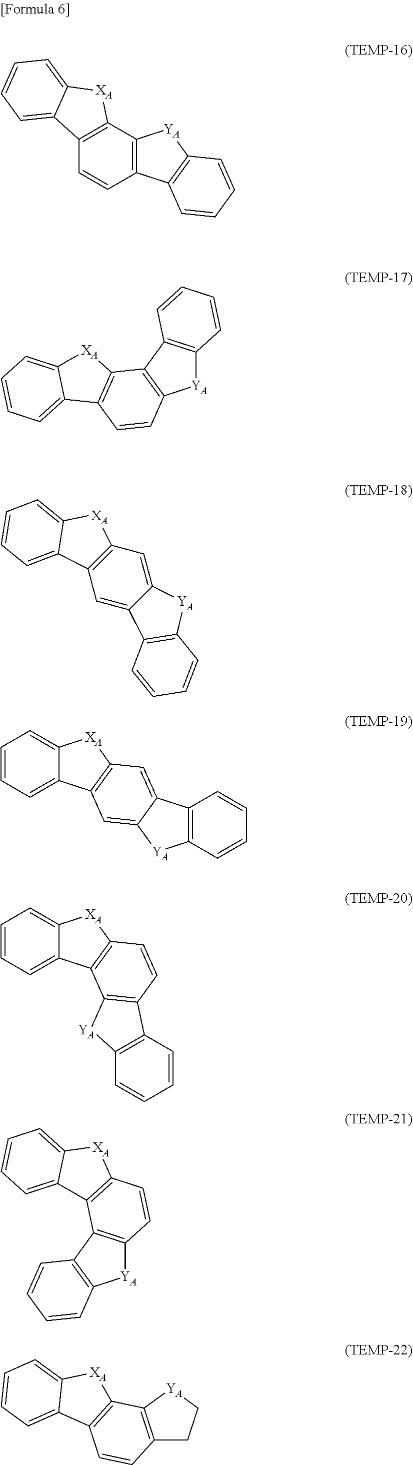

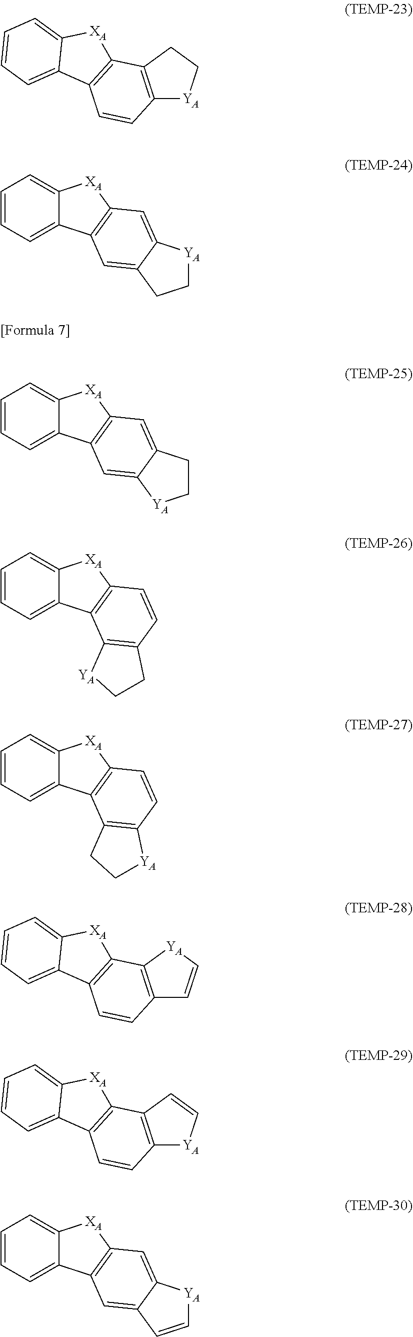

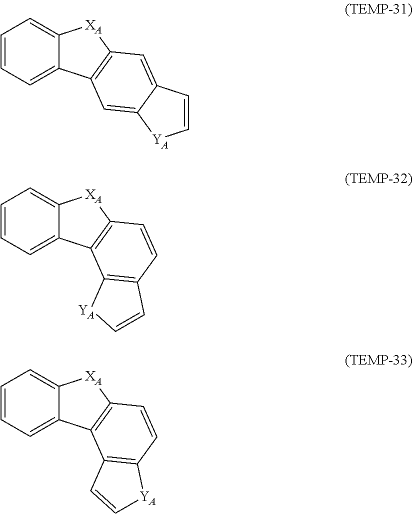

In the formulae (TEMP-16) to (TEMP-33), X_{A} and Y_{A} are each independently an oxygen atom, a sulfur atom, NH, or CH₂. However, at least one of X_{A} or Y_{A} is an oxygen atom, a sulfur atom, or NH.

When at least one of X_{A} Or Y_{A} in the formulae (TEMP-16) to (TEMP-33) is NH or CH₂, the monovalent heterocyclic groups derived from the cyclic structures represented by the formulae (TEMP-16) to (TEMP-33) include a monovalent group derived by removing one hydrogen atom from NH, or CH₂.

Substituted Heterocyclic Groups Including Nitrogen Atom (Specific Example Group G2B1):
(9-phenyl)carbazolyl group, (9-biphenylyl)carbazolyl group, (9-phenyl)phenylcarbazolyl group, (9-naphthyl)carbazolyl group, diphenylcarbazole-9-yl group, phenylcarbazole-9-yl group, methylbenzimidazolyl group, ethylbenzimidazolyl group, phenyltriazinyl group, biphenylyltriazinyl group, diphenyltriazinyl group, phenylquinazolinyl group, and biphenylquinazolinyl group.

Substituted Heterocyclic Groups Including Oxygen Atom (Specific Example Group G2B2):
phenyldibenzofuranyl group, methyldibenzofuranyl group, t-butyldibenzofuranyl group, and monovalent residue of spiro[9H-xanthene-9,9′-[9H]fluorene].

Substituted Heterocyclic Groups Including Sulfur Atom (Specific Example Group G2B3):
phenyldibenzothiophenyl group, methyldibenzothiophenyl group, t-butyldibenzothiophenyl group, and monovalent residue of spiro[9H-thioxanthene-9,9′-[9H]fluorene].

Groups Obtained by Substituting at Least One Hydrogen Atom of Monovalent Heterocyclic Group Derived from Cyclic Structures Represented by Formulae (TEMP-16) to (TEMP-33) with Substituent (Specific Example Group G2B4):

The “at least one hydrogen atom of a monovalent heterocyclic group” means at least one hydrogen atom selected from a hydrogen atom bonded to a ring carbon atom of the monovalent heterocyclic group, a hydrogen atom bonded to a nitrogen atom of at least one of X_{A} or Y_{A} in a form of NH, and a hydrogen atom of one of X_{A} and Y_{A} in a form of a methylene group (CH₂).

Substituted or Unsubstituted Alkyl Group

Specific examples (specific example group G3) of the “substituted or unsubstituted alkyl group” mentioned herein include unsubstituted alkyl groups (specific example group G3A) and substituted alkyl groups (specific example group G3B below). Herein, an unsubstituted alkyl group refers to an “unsubstituted alkyl group” in a “substituted or unsubstituted alkyl group,” and a substituted alkyl group refers to a “substituted alkyl group” in a “substituted or unsubstituted alkyl group.” A simply termed “alkyl group” herein includes both of an “unsubstituted alkyl group” and a “substituted alkyl group.”

The “substituted alkyl group” refers to a group derived by substituting at least one hydrogen atom in an “unsubstituted alkyl group” with a substituent. Specific examples of the “substituted alkyl group” include a group derived by substituting at least one hydrogen atom of an “unsubstituted alkyl group” (specific example group G3A) below with a substituent, and examples of the substituted alkyl group (specific example group G3B) below. Herein, the alkyl group for the “unsubstituted alkyl group” refers to a chain alkyl group. Accordingly, the “unsubstituted alkyl group” include linear “unsubstituted alkyl group” and branched “unsubstituted alkyl group.” It should be noted that the examples of the “unsubstituted alkyl group” and the “substituted alkyl group” mentioned herein are merely exemplary, and the “substituted alkyl group” mentioned herein includes a group derived by further substituting a hydrogen atom of a skeleton of the “substituted alkyl group” in the specific example group G3B, and a group derived by further substituting a hydrogen atom of a substituent of the “substituted alkyl group” in the specific example group G3B.

Unsubstituted Alkyl Group (Specific Example Group G3A):
methyl group, ethyl group, n-propyl group, isopropyl group, n-butyl group, isobutyl group, s-butyl group, and t-butyl group.

Substituted Alkyl Group (Specific Example Group G3B):
heptafluoropropyl group (including isomer thereof), pentafluoroethyl group, 2,2,2-trifluoroethyl group, and trifluoromethyl group.

Substituted or Unsubstituted Alkenyl Group

Specific examples (specific example group G4) of the “substituted or unsubstituted alkenyl group” mentioned herein include unsubstituted alkenyl groups (specific example group G4A) and substituted alkenyl groups (specific example group G4B). Herein, an unsubstituted alkenyl group refers to an “unsubstituted alkenyl group” in a “substituted or unsubstituted alkenyl group,” and a substituted alkenyl group refers to a “substituted alkenyl group” in a “substituted or unsubstituted alkenyl group.” A simply termed “alkenyl group” herein includes both of an “unsubstituted alkenyl group” and a “substituted alkenyl group.”

The “substituted alkenyl group” refers to a group derived by substituting at least one hydrogen atom in an “unsubstituted alkenyl group” with a substituent. Specific examples of the “substituted alkenyl group” include an “unsubstituted alkenyl group” (specific example group G4A) substituted by a substituent, and examples of the substituted alkenyl group (specific example group G4B) below. It should be noted that the examples of the “unsubstituted alkenyl group” and the “substituted alkenyl group” mentioned herein are merely exemplary, and the “substituted alkenyl group” mentioned herein includes a group derived by further substituting a hydrogen atom of a skeleton of the “substituted alkenyl group” in the specific example group G4B with a substituent, and a group derived by further substituting a hydrogen atom of a substituent of the “substituted alkenyl group” in the specific example group G4B with a substituent.

Unsubstituted Alkenyl Group (Specific Example Group G4A):
vinyl group, allyl group, 1-butenyl group, 2-butenyl group, and 3-butenyl group.

Substituted Alkenyl Group (Specific Example Group G4B):
1,3-butanedienyl group, 1-methylvinyl group, 1-methylallyl group, 1,1-dimethylallyl group, 2-methylallyl group, and 1,2-dimethylallyl group.

Substituted or Unsubstituted Alkynyl Group

Specific examples (specific example group G5) of the “substituted or unsubstituted alkynyl group” mentioned herein include unsubstituted alkynyl groups (specific example group G5A) below. Herein, an unsubstituted alkynyl group refers to an “unsubstituted alkynyl group” in a “substituted or unsubstituted alkynyl group.” A simply termed “alkynyl group” herein includes both of an “unsubstituted alkynyl group” and a “substituted alkynyl group.”

The “substituted alkynyl group” refers to a group derived by substituting at least one hydrogen atom in an “unsubstituted alkynyl group” with a substituent. Specific examples of the “substituted alkynyl group” include a group derived by substituting at least one hydrogen atom of the “unsubstituted alkynyl group” (specific example group G5A) below with a substituent.

Unsubstituted Alkynyl Group (Specific Example Group G5A): ethynyl group.

Substituted or Unsubstituted Cycloalkyl Group

Specific examples (specific example group G6) of the “substituted or unsubstituted cycloalkyl group” mentioned herein include unsubstituted cycloalkyl groups (specific example group G6A) and substituted cycloalkyl groups (specific example group G6B). Herein, an unsubstituted cycloalkyl group refers to an “unsubstituted cycloalkyl group” in a “substituted or unsubstituted cycloalkyl group,” and a substituted cycloalkyl group refers to a “substituted cycloalkyl group” in a “substituted or unsubstituted cycloalkyl group.” A simply termed “cycloalkyl group” herein includes both of an “unsubstituted cycloalkyl group” and a “substituted cycloalkyl group.”

The “substituted cycloalkyl group” refers to a group derived by substituting at least one hydrogen atom of an “unsubstituted cycloalkyl group” with a substituent. Specific examples of the “substituted cycloalkyl group” include a group derived by substituting at least one hydrogen atom of the “unsubstituted cycloalkyl group” (specific example group G6A) below with a substituent, and examples of the substituted cycloalkyl group (specific example group G6B) below. It should be noted that the examples of the “unsubstituted cycloalkyl group” and the “substituted cycloalkyl group” mentioned herein are merely exemplary, and the “substituted cycloalkyl group” mentioned herein includes a group derived by substituting at least one hydrogen atom bonded to a carbon atom of a skeleton of the “substituted cycloalkyl group” in the specific example group G6B with a substituent, and a group derived by further substituting a hydrogen atom of a substituent of the “substituted cycloalkyl group” in the specific example group G6B with a substituent.

Unsubstituted Cycloalkyl Group (Specific Example Group G6A):
cyclopropyl group, cyclobutyl group, cyclopentyl group, cyclohexyl group, 1-adamantyl group, 2-adamantyl group, 1-norbornyl group, and 2-norbornyl group.

Substituted Cycloalkyl Group (Specific Example Group G6B):
4-methylcyclohexyl group

Group represented by —Si(R₉₀₁)(R₉₀₂)(R₉₀₃) Specific examples (specific example group G7) of the group represented herein by —Si(R₉₀₁)(R₉₀₂)(R₉₀₃) include: —Si(G1)(G1)(G1); —Si(G1)(G2)(G2); —Si(G1)(G1)(G2); —Si(G2)(G2)(G2); —Si(G3)(G3)(G3); and —Si(G6)(G6)(G6).

Herein: G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3; and
G6 represents a “substituted or unsubstituted cycloalkyl group” in the specific example group G6.

A plurality of G1 in —Si(G1)(G1)(G1) are mutually the same or different.

A plurality of G2 in —Si(G1)(G2)(G2) are mutually the same or different.

A plurality of G1 in —Si(G1)(G1)(G2) are mutually the same or different.

A plurality of G2 in —Si(G2)(G2)(G2) are mutually the same or different.

The plurality of G3 in —Si(G3)(G3)(G3) are mutually the same or different.

A plurality of G6 in —Si(G6)(G6)(G6) are mutually the same or different.

Group Represented by —O—(R₉₀₄)

Specific examples (specific example group G8) of a group represented by —O—(R₉₀₄) herein include: —O(G1); —O(G2); —O(G3); and —O(G6).

Herein: G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3; and
G6 represents a “substituted or unsubstituted cycloalkyl group” in the specific example group G6.

Group Represented by —S—(R₉₀₅)

Specific examples (specific example group G9) of a group represented herein by —S—(R₉₀₅) include: —S(G1); —S(G2); —S(G3); and —S(G6).

Herein: G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3; and
G6 represents a “substituted or unsubstituted cycloalkyl group” in the specific example group G6.

Group Represented by —N(R₉₀₆)(R₉₀₇)

Specific examples (specific example group G10) of a group represented herein by —N(R₉₀₆)(R₉₀₇) include: —N(G1)(G1); —N(G2)(G2); —N(G1)(G2); —N(G3)(G3); and —N(G6)(G6).

Herein: G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3; and
G6 represents a “substituted or unsubstituted cycloalkyl group” in the specific example group G6.

A plurality of G1 in —N(G1)(G1) are mutually the same or different.

A plurality of G2 in —N(G2)(G2) are mutually the same or different.

A plurality of G3 in —N(G3)(G3) are mutually the same or different.

A plurality of G6 in —N(G6)(G6)) are mutually the same or different.

Halogen Atom

Specific examples (specific example group G11) of “halogen atom” mentioned herein include a fluorine atom, chlorine atom, bromine atom, and iodine atom.

Substituted or Unsubstituted Fluoroalkyl Group

The “substituted or unsubstituted fluoroalkyl group” mentioned herein refers to a group derived by substituting at least one hydrogen atom bonded to at least one of carbon atoms forming an alkyl group in the “substituted or unsubstituted alkyl group” with a fluorine atom, and also includes a group (perfluoro group) derived by substituting all of hydrogen atoms bonded to carbon atoms forming the alkyl group in the “substituted or unsubstituted alkyl group” with fluorine atoms. An “unsubstituted fluoroalkyl group” has, unless otherwise specified herein, 1 to 50 carbon atoms, preferably 1 to 30 carbon atoms, more preferably 1 to 18 carbon atoms. The “substituted fluoroalkyl group” refers to a group derived by substituting at least one hydrogen atom in a “fluoroalkyl group” with a substituent. It should be noted that the examples of the “substituted fluoroalkyl group” mentioned herein include a group derived by further substituting at least one hydrogen atom bonded to a carbon atom of an alkyl chain of a “substituted fluoroalkyl group” with a substituent, and a group derived by further substituting at least one hydrogen atom of a substituent of the “substituted fluoroalkyl group” with a substituent. Specific examples of the “substituted fluoroalkyl group” include a group derived by substituting at least one hydrogen atom of the “alkyl group” (specific example group G3) with a fluorine atom.

Substituted or Unsubstituted Haloalkyl Group

The “substituted or unsubstituted haloalkyl group” mentioned herein refers to a group derived by substituting at least one hydrogen atom bonded to carbon atoms forming the alkyl group in the “substituted or unsubstituted alkyl group” with a halogen atom, and also includes a group derived by substituting all hydrogen atoms bonded to carbon atoms forming the alkyl group in the “substituted or unsubstituted alkyl group” with halogen atoms. An “unsubstituted haloalkyl group” has, unless otherwise specified herein, 1 to 50 carbon atoms, preferably 1 to 30 carbon atoms, more preferably 1 to 18 carbon atoms. The “substituted haloalkyl group” refers to a group derived by substituting at least one hydrogen atom in a “haloalkyl group” with a substituent. It should be noted that the examples of the “substituted haloalkyl group” mentioned herein include a group derived by further substituting at least one hydrogen atom bonded to a carbon atom of an alkyl chain of a “substituted haloalkyl group” with a substituent, and a group derived by further substituting at least one hydrogen atom of a substituent of the “substituted haloalkyl group” with a substituent. Specific examples of the “substituted haloalkyl group” include a group derived by substituting at least one hydrogen atom of the “alkyl group” (specific example group G3) with a halogen atom. The haloalkyl group is sometimes referred to as a halogenated alkyl group.

Substituted or Unsubstituted Alkoxy Group

Specific examples of a “substituted or unsubstituted alkoxy group” mentioned herein include a group represented by —O(G3), G3 being the “substituted or unsubstituted alkyl group” in the specific example group G3. An “unsubstituted alkoxy group” has, unless otherwise specified herein, 1 to 50 carbon atoms, preferably 1 to 30 carbon atoms, more preferably 1 to 18 carbon atoms.

Substituted or Unsubstituted Alkylthio Group

Specific examples of a “substituted or unsubstituted alkylthio group” mentioned herein include a group represented by —S(G3), G3 being the “substituted or unsubstituted alkyl group” in the specific example group G3. An “unsubstituted alkylthio group” has, unless otherwise specified herein, 1 to 50 carbon atoms, preferably 1 to 30 carbon atoms, more preferably 1 to 18 carbon atoms.

Substituted or Unsubstituted Aryloxy Group

Specific examples of a “substituted or unsubstituted aryloxy group” mentioned herein include a group represented by —O(G1), G1 being the “substituted or unsubstituted aryl group” in the specific example group G1. An “unsubstituted aryloxy group” has, unless otherwise specified herein, 6 to 50 ring carbon atoms, preferably 6 to 30 ring carbon atoms, more preferably 6 to 18 ring carbon atoms.

Substituted or Unsubstituted Arylthio Group

Specific examples of a “substituted or unsubstituted arylthio group” mentioned herein include a group represented by —S(G1), G1 being the “substituted or unsubstituted aryl group” in the specific example group G1. An “unsubstituted arylthio group” has, unless otherwise specified herein, 6 to 50 ring carbon atoms, preferably 6 to 30 ring carbon atoms, more preferably 6 to 18 ring carbon atoms.

Substituted or Unsubstituted Trialkylsilyl Group

Specific examples of a “trialkylsilyl group” mentioned herein include a group represented by —Si(G3)(G3)(G3), G3 being the “substituted or unsubstituted alkyl group” in the specific example group G3. The plurality of G3 in —Si(G3)(G3)(G3) are mutually the same or different. Each of the alkyl groups in the “trialkylsilyl group” has, unless otherwise specified herein, 1 to 50 carbon atoms, preferably 1 to 20 carbon atoms, more preferably 1 to 6 carbon atoms.

Substituted or Unsubstituted Aralkyl Group

Specific examples of a “substituted or unsubstituted aralkyl group” mentioned herein include a group represented by (G3)-(G1), G3 being the “substituted or unsubstituted alkyl group” in the specific example group G3, G1 being the “substituted or unsubstituted aryl group” in the specific example group G1. Accordingly, the “aralkyl group” is a group derived by substituting a hydrogen atom of the “alkyl group” with a substituent in a form of the “aryl group,” which is an example of the “substituted alkyl group.” An “unsubstituted aralkyl group,” which is an “unsubstituted alkyl group” substituted by an “unsubstituted aryl group,” has, unless otherwise specified herein, 7 to 50 carbon atoms, preferably 7 to 30 carbon atoms, more preferably 7 to 18 carbon atoms.

Specific examples of the “substituted or unsubstituted aralkyl group” include a benzyl group, 1-phenylethyl group, 2-phenylethyl group, 1-phenylisopropyl group, 2-phenylisopropyl group, phenyl-t-butyl group, α-naphthylmethyl group, 1-α-naphthylethyl group, 2-α-naphthylethyl group, 1-α-naphthylisopropyl group, 2-α-naphthylisopropyl group, β-naphthylmethyl group, 1-β-naphthylethyl group, 2-β-naphthylethyl group, 1-β-naphthylisopropyl group, and 2-β-naphthylisopropyl group.

Preferable examples of the substituted or unsubstituted aryl group mentioned herein include, unless otherwise specified herein, a phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, phenanthryl group, pyrenyl group, chrysenyl group, triphenylenyl group, fluorenyl group, 9,9′-spirobifluorenyl group, 9,9-dimethylfluorenyl group, and 9,9-diphenylfluorenyl group.

Preferable examples of the substituted or unsubstituted heterocyclic group mentioned herein include, unless otherwise specified herein, a pyridyl group, pyrimidinyl group, triazinyl group, quinolyl group, isoquinolyl group, quinazolinyl group, benzimidazolyl group, phenanthrolinyl group, carbazolyl group (1-carbazolyl group, 2-carbazolyl group, 3-carbazolyl group, 4-carbazolyl group, or 9-carbazolyl group), benzocarbazolyl group, azacarbazolyl group, diazacarbazolyl group, dibenzofuranyl group, naphthobenzofuranyl group, azadibenzofuranyl group, diazadibenzofuranyl group, dibenzothiophenyl group, naphthobenzothiophenyl group, azadibenzothiophenyl group, diazadibenzothiophenyl group, (9-phenyl)carbazolyl group ((9-phenyl)carbazole-1-yl group, (9-phenyl)carbazole-2-yl group, (9-phenyl)carbazole-3-yl group, or (9-phenyl)carbazole-4-yl group), (9-biphenylyl)carbazolyl group, (9-phenyl)phenylcarbazolyl group, diphenylcarbazole-9-yl group, phenylcarbazole-9-yl group, phenyltriazinyl group, biphenylyltriazinyl group, diphenyltriazinyl group, phenyldibenzofuranyl group, and phenyldibenzothiophenyl group.

The carbazolyl group mentioned herein is, unless otherwise specified herein, specifically a group represented by one of formulae below.

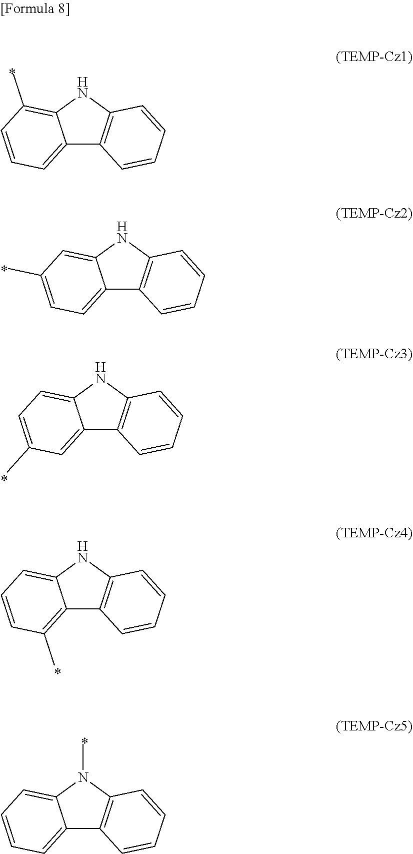

The (9-phenyl)carbazolyl group mentioned herein is, unless otherwise specified herein, specifically a group represented by one of formulae below.

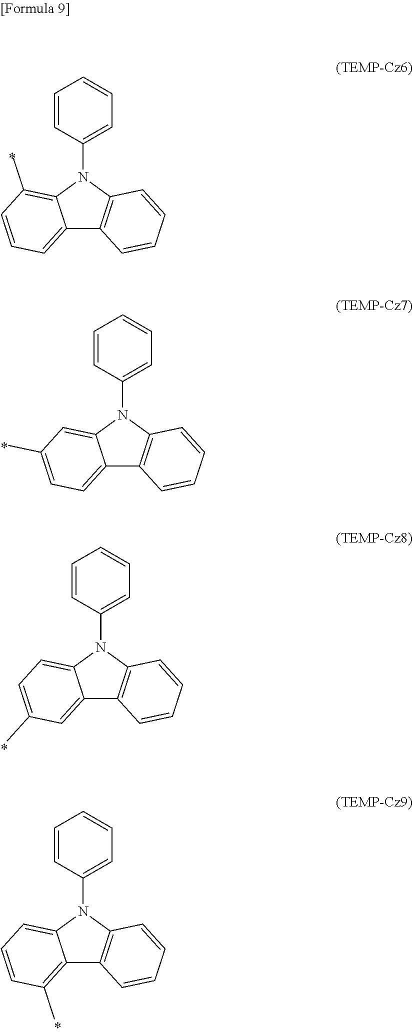 [SMILES: Cc1cccc2c3ccccc3n(-c3ccccc3)c12 ; Cc1ccc2c3ccccc3n(-c3ccccc3)c2c1 ; Cc1ccc2c(c1)c1ccccc1n2-c1ccccc1 ; Cc1cccc2c1c1ccccc1n2-c1ccccc1]

In the formulae (TEMP-Cz1) to (TEMP-Cz9), * represents a bonding position.

The dibenzofuranyl group and dibenzothiophenyl group mentioned herein are, unless otherwise specified herein, each specifically represented by one of formulae below.

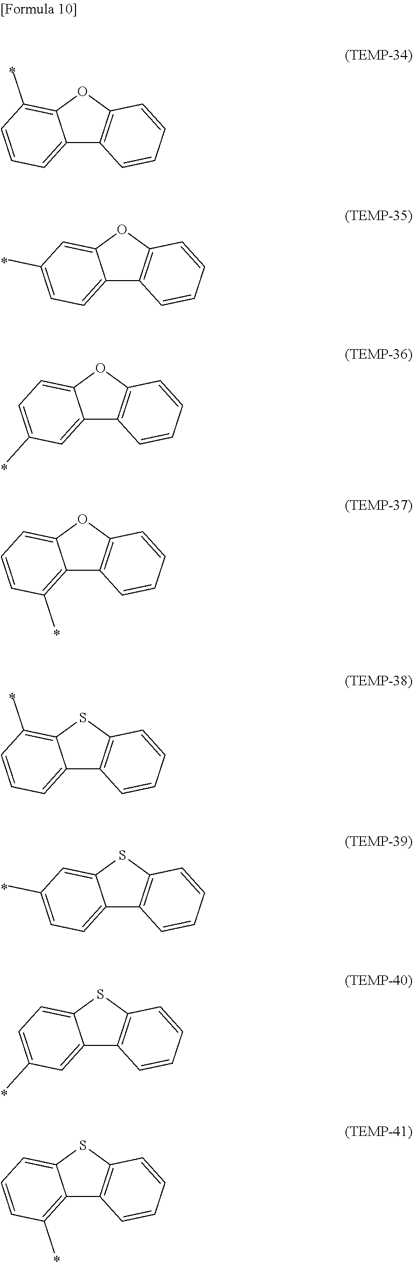

In the formulae (TEMP-34) to (TEMP-41), * represents a bonding position.

Preferable examples of the substituted or unsubstituted alkyl group mentioned herein include, unless otherwise specified herein, a methyl group, ethyl group, propyl group, isopropyl group, n-butyl group, isobutyl group, and t-butyl group.

Substituted or Unsubstituted Arylene Group

The “substituted or unsubstituted arylene group” mentioned herein is, unless otherwise specified herein, a divalent group derived by removing one hydrogen atom on an aryl ring of the “substituted or unsubstituted aryl group.” Specific examples of the “substituted or unsubstituted arylene group” (specific example group G12) include a divalent group derived by removing one hydrogen atom on an aryl ring of the “substituted or unsubstituted aryl group” in the specific example group G1.

Substituted or Unsubstituted Divalent Heterocyclic Group

The “substituted or unsubstituted divalent heterocyclic group” mentioned herein is, unless otherwise specified herein, a divalent group derived by removing one hydrogen atom on a heterocyclic ring of the “substituted or unsubstituted heterocyclic group.” Specific examples of the “substituted or unsubstituted heterocyclic group” (specific example group G13) include a divalent group derived by removing one hydrogen atom on a heterocyclic ring of the “substituted or unsubstituted heterocyclic group” in the specific example group G2.

Substituted or Unsubstituted Alkylene Group

The “substituted or unsubstituted alkylene group” mentioned herein is, unless otherwise specified herein, a divalent group derived by removing one hydrogen atom on an alkyl ring of the “substituted or unsubstituted alkyl group.” Specific examples of the “substituted or unsubstituted alkylene group” (specific example group G14) include a divalent group derived by removing one hydrogen atom on an alkyl ring of the “substituted or unsubstituted alkyl group” in the specific example group G3.

The substituted or unsubstituted arylene group mentioned herein is, unless otherwise specified herein, preferably any one of groups represented by formulae (TEMP-42) to (TEMP-68) below.

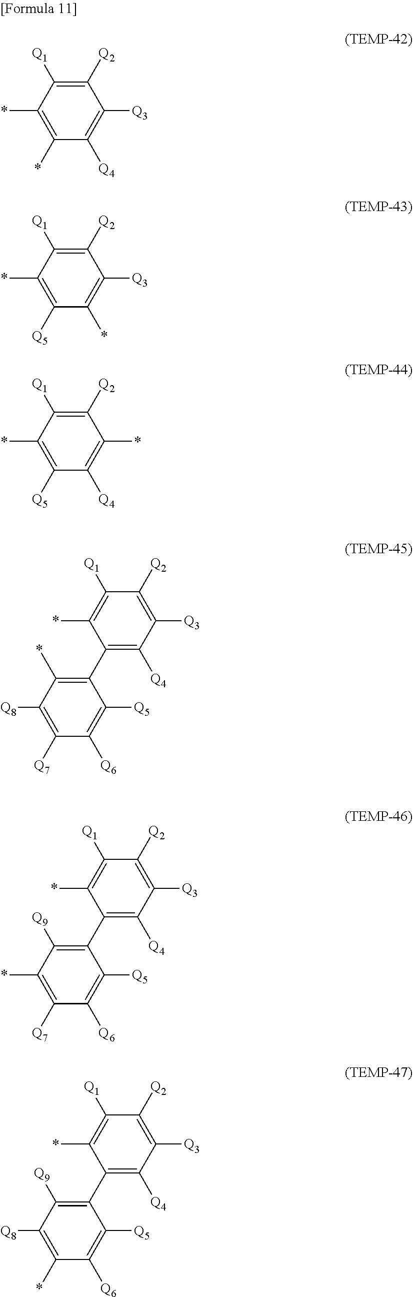

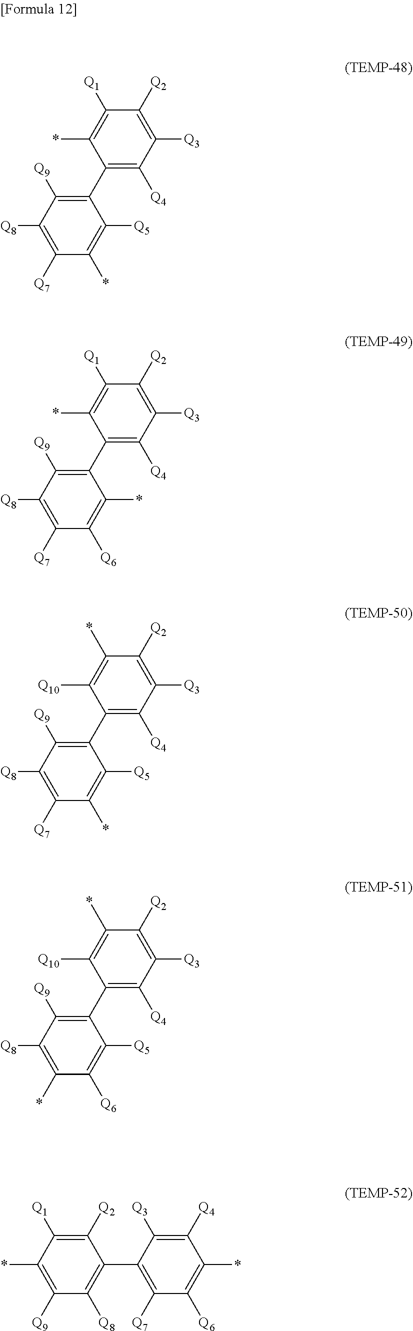

In the formulae (TEMP-42) to (TEMP-52), Q₁ to Q₁₀ each independently are a hydrogen atom or a substituent.

In the formulae (TEMP-42) to (TEMP-52), * represents a bonding position.

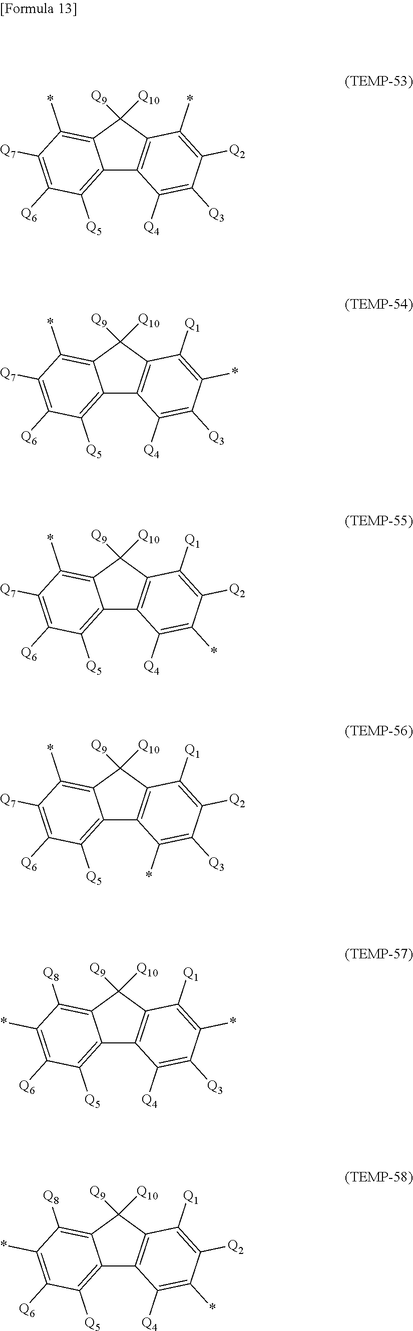

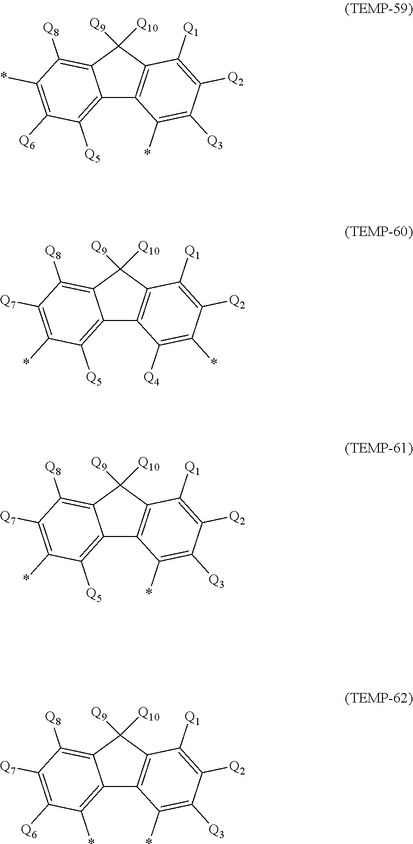

In the formulae (TEMP-53) to (TEMP-62), Q₁ to Q₁₀ each independently are a hydrogen atom or a substituent.

In the formulae, Q₉ and Q₁₀ may be mutually bonded through a single bond to form a ring.

In the formulae (TEMP-53) to (TEMP-62), * represents a bonding position.

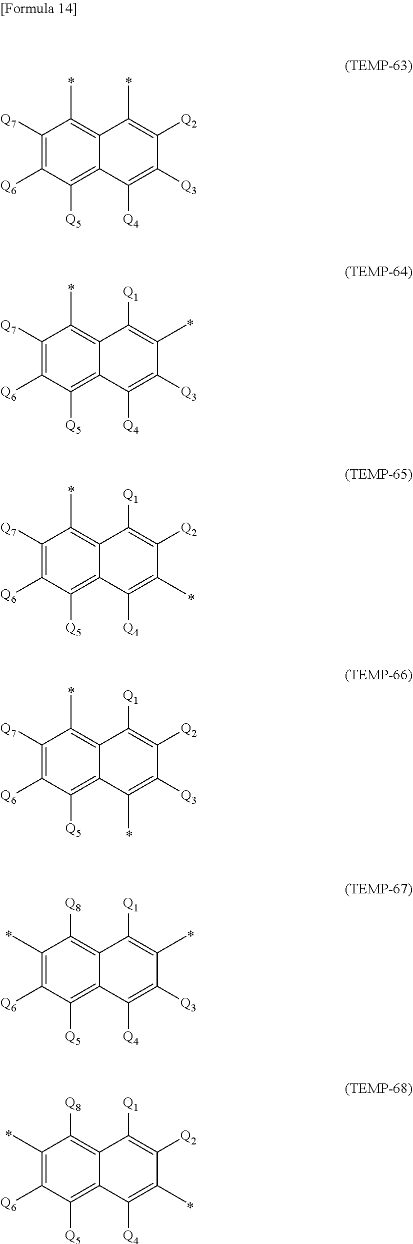

In the formulae (TEMP-63) to (TEMP-68), Q₁ to Q₈ each independently are a hydrogen atom or a substituent.

In the formulae (TEMP-63) to (TEMP-68), * represents a bonding position.

The substituted or unsubstituted divalent heterocyclic group mentioned herein is, unless otherwise specified herein, preferably a group represented by any one of formulae (TEMP-69) to (TEMP-102) below.

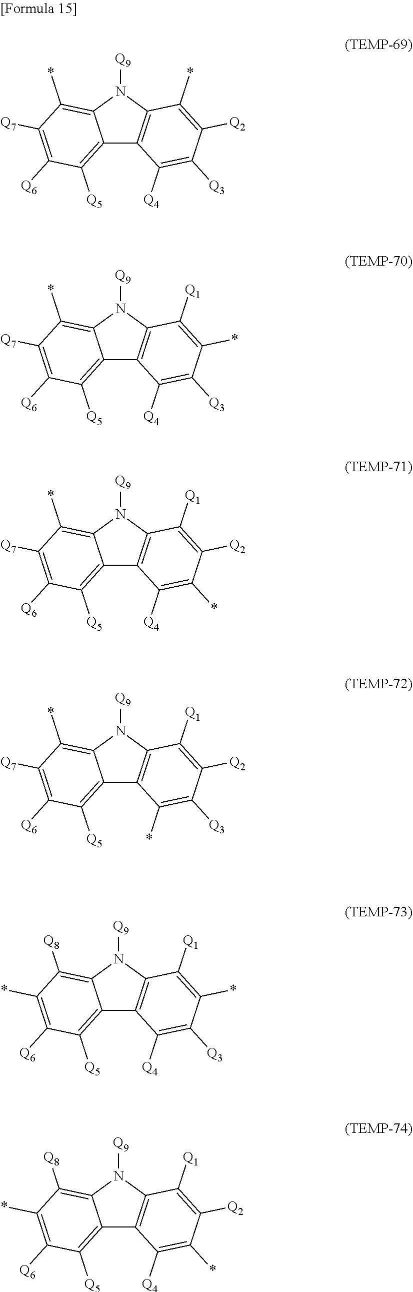

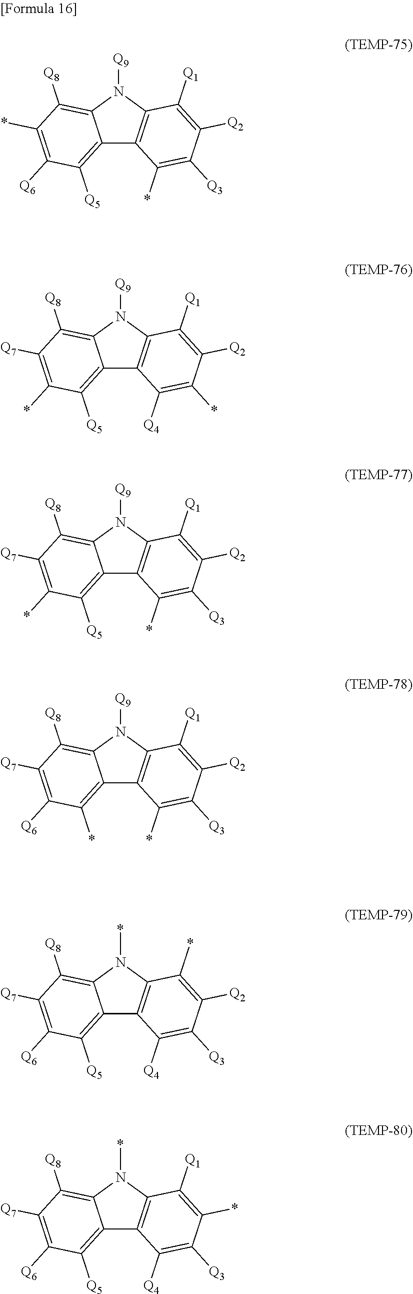

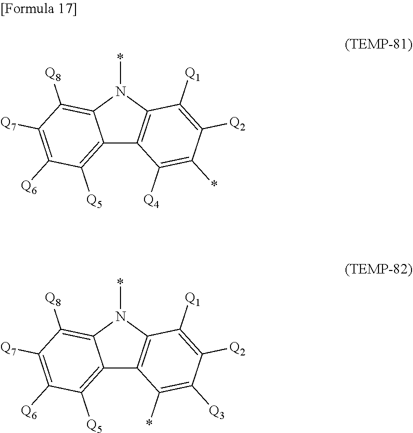

In the formulae (TEMP-69) to (TEMP-82), Q₁ to Q₉ each independently are a hydrogen atom or a substituent.

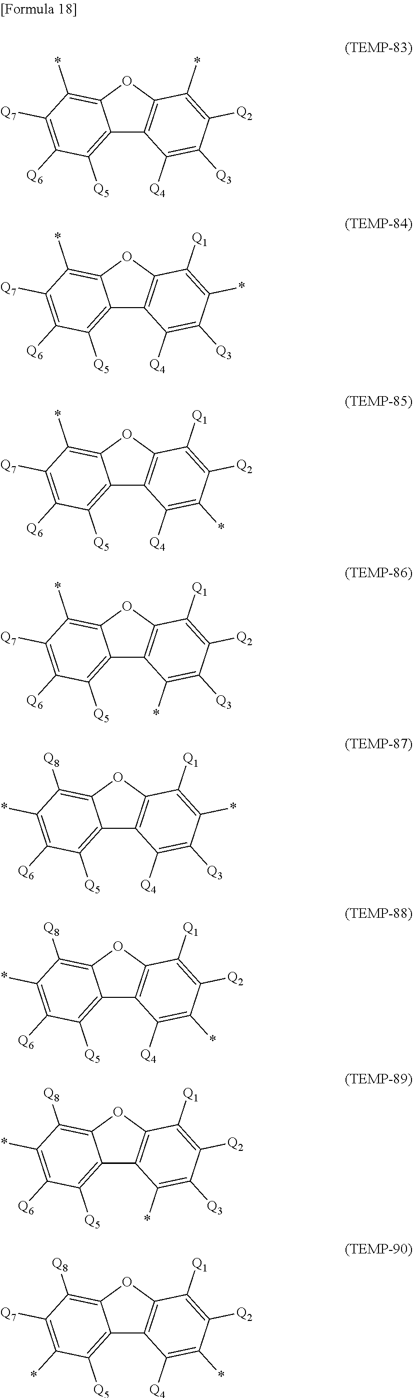

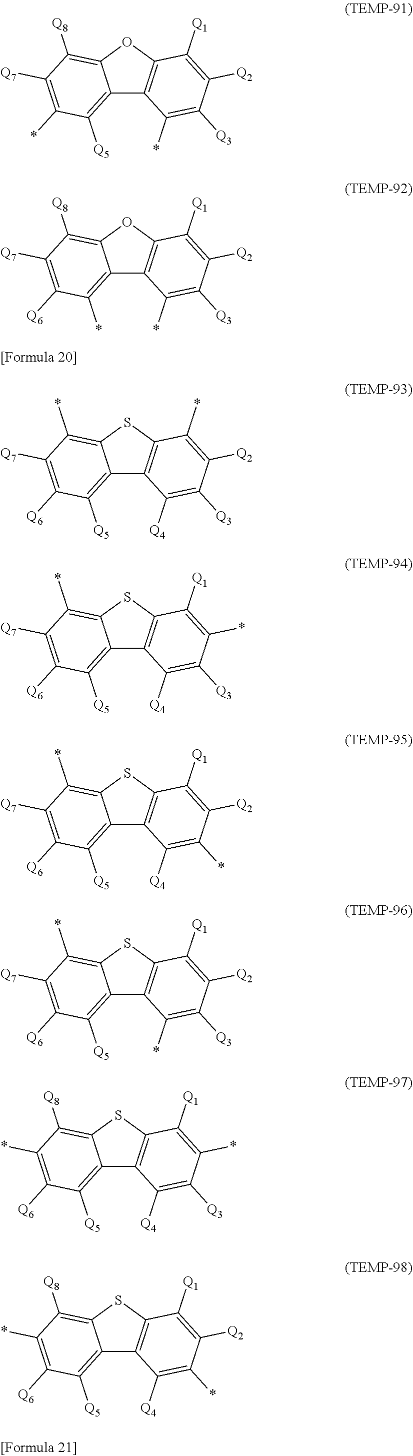

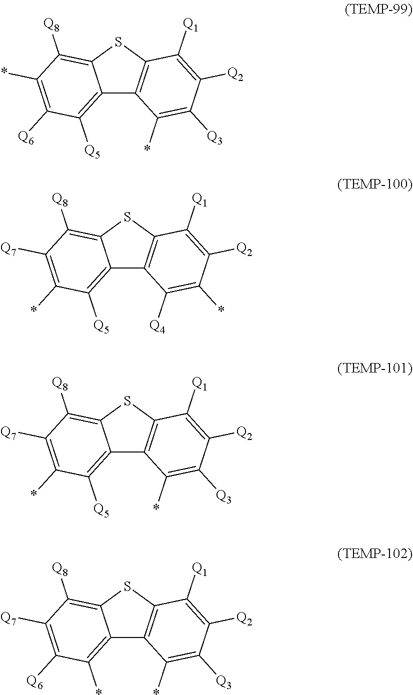

In the formulae (TEMP-83) to (TEMP-102), Q₁ to Q₈ each independently are a hydrogen atom or a substituent.

The substituent mentioned herein has been described above.

Instance of “Bonded to Form Ring”

Instances where “at least one combination of adjacent two or more (of . . . ) are mutually bonded to form a substituted or unsubstituted monocyclic ring, mutually bonded to form a substituted or unsubstituted fused ring, or not mutually bonded” mentioned herein refer to instances where “at least one combination of adjacent two or more (of . . . ) are mutually bonded to form a substituted or unsubstituted monocyclic ring,” at least one combination of adjacent two or more (of . . . ) are mutually bonded to form a substituted or unsubstituted fused ring,” and “at least one combination of adjacent two or more (of . . . ) are not mutually bonded.”

Instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (these instances will be sometimes collectively referred to as an instance of “bonded to form a ring” hereinafter) will be described below. An anthracene compound having a basic skeleton in a form of an anthracene ring and represented by a formula (TEMP-103) below will be used as an example for the description.

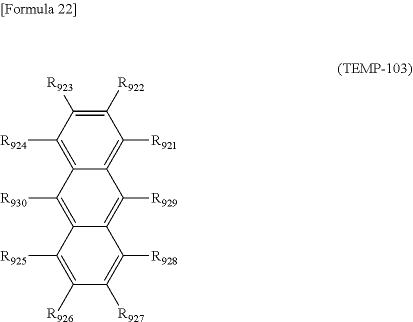

For instance, when “at least one combination of adjacent two or more of” R₉₂₁ to R₉₃₀ “are mutually bonded to form a ring,” the combination of adjacent ones of R₉₂₁ to R₉₃₀ (i.e. the combination at issue) is a combination of R₉₂₁ and a combination of R₉₂₂, R₉₂₂ and R₉₂₃, a combination of R₉₂₃ and R₉₂₄, a combination of R₉₂₄ and R₉₃₀, a combination of R₉₃₀ and R₉₂₅, a combination of R₉₂₅ and R₉₂₅, a combination of R₉₂₅ and R₉₂₇, a combination of R₉₂₇ and R₉₂₅, a combination of R₉₂₈ and R₉₂₉, or a combination of R₉₂₉ and R₉₂₁.

The term “at least one combination” means that two or more of the above combinations of adjacent two or more of R₉₂₁ to R₉₃₀ may simultaneously form rings. For instance, when R₉₂₁ and R₉₂₂ are mutually bonded to form a ring Q_{A} and R₉₂₅ and R₉₂₆ are simultaneously mutually bonded to form a ring Q_{B}, the anthracene compound represented by the formula (TEMP-103) is represented by a formula (TEMP-104) below.

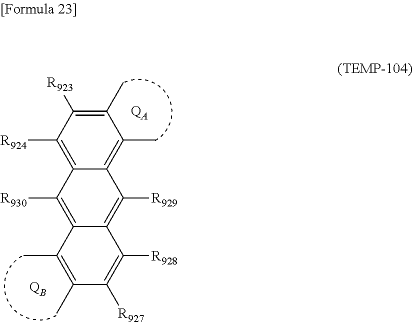

The instance where the “combination of adjacent two or more” form a ring means not only an instance where the “two” adjacent components are bonded but also an instance where adjacent “three or more” are bonded. For instance, R₉₂₁ and R₉₂₂ are mutually bonded to form a ring Q_{A} and R₉₂₂, R₉₂₃ are mutually bonded to form a ring Q_{C}, and mutually adjacent three components (R₉₂₁, R₉₂₂ and R₉₂₃) are mutually bonded to form a ring fused to the anthracene basic skeleton. In this case, the anthracene compound represented by the formula (TEMP-103) is represented by a formula (TEMP-105) below. In the formula (TEMP-105) below, the ring Q_{A} and the ring Q_{C} share R₉₂₂.

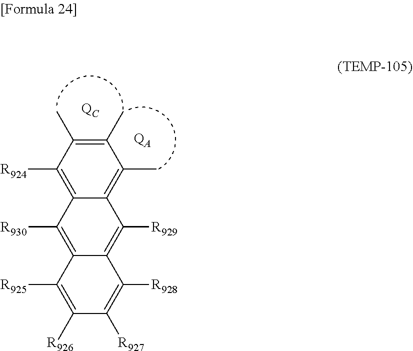

The formed “monocyclic ring” or “fused ring” may be, in terms of the formed ring in itself, a saturated ring or an unsaturated ring. When the “combination of adjacent two” form a “monocyclic ring” or a “fused ring,” the “monocyclic ring” or “fused ring” may be a saturated ring or an unsaturated ring. For instance, the ring Q_{A} and the ring Q_{B} formed in the formula (TEMP-104) are each independently a “monocyclic ring” or a “fused ring.” Further, the ring Q_{A} and the ring Q_{C} formed in the formula (TEMP-105) are each a “fused ring.” The ring Q_{A} and the ring Q_{C} in the formula (TEMP-105) are fused to form a fused ring. When the ring Q_{A} in the formula (TMEP-104) is a benzene ring, the ring Q_{A} is a monocyclic ring. When the ring Q_{A} in the formula (TMEP-104) is a naphthalene ring, the ring Q_{A} is a fused ring.

The “unsaturated ring” represents an aromatic hydrocarbon ring or an aromatic heterocycle. The “saturated ring” represents an aliphatic hydrocarbon ring or a non-aromatic heterocycle.

Specific examples of the aromatic hydrocarbon ring include a ring formed by terminating a bond of a group in the specific example of the specific example group G1 with a hydrogen atom.

Specific examples of the aromatic heterocyclic ring include a ring formed by terminating a bond of an aromatic heterocyclic group in the specific example of the specific example group G2 with a hydrogen atom.

Specific examples of the aliphatic hydrocarbon ring include a ring formed by terminating a bond of a group in the specific example of the specific example group G6 with a hydrogen atom.

The phrase “to form a ring” herein means that a ring is formed only by a plurality of atoms of a basic skeleton, or by a combination of a plurality of atoms of the basic skeleton and one or more optional atoms. For instance, the ring Q_{A} formed by mutually bonding R₉₂₁ and R₉₂₂ shown in the formula (TEMP-104) is a ring formed by a carbon atom of the anthracene skeleton bonded with R₉₂₁, a carbon atom of the anthracene skeleton bonded with R₉₂₂, and one or more optional atoms. Specifically, when the ring Q_{A} is a monocyclic unsaturated ring formed by R₉₂₁ and R₉₂₂, the ring formed by a carbon atom of the anthracene skeleton bonded with R₉₂₁, a carbon atom of the anthracene skeleton bonded with R₉₂₂, and four carbon atoms is a benzene ring.

The “optional atom” is, unless otherwise specified herein, preferably at least one atom selected from the group consisting of a carbon atom, nitrogen atom, oxygen atom, and sulfur atom. A bond of the optional atom (e.g. a carbon atom and a nitrogen atom) not forming a ring may be terminated by a hydrogen atom or the like or may be substituted by an “optional substituent” described later. When the ring includes an optional element other than carbon atom, the resultant ring is a heterocycle.

The number of “one or more optional atoms” forming the monocyclic ring or fused ring is, unless otherwise specified herein, preferably in a range from 2 to 15, more preferably in a range from 3 to 12, further preferably in a range from 3 to 5.

Unless otherwise specified herein, the ring, which may be a “monocyclic ring” or “fused ring,” is preferably a “monocyclic ring.”

Unless otherwise specified herein, the ring, which may be a “saturated ring” or “unsaturated ring,” is preferably an “unsaturated ring.”

Unless otherwise specified herein, the “monocyclic ring” is preferably a benzene ring.

Unless otherwise specified herein, the “unsaturated ring” is preferably a benzene ring.

When “at least one combination of adjacent two or more” (of . . . ) are “mutually bonded to form a substituted or unsubstituted monocyclic ring” or “mutually bonded to form a substituted or unsubstituted fused ring,” unless otherwise specified herein, at least one combination of adjacent two or more of components are preferably mutually bonded to form a substituted or unsubstituted “unsaturated ring” formed of a plurality of atoms of the basic skeleton, and 1 to 15 atoms of at least one element selected from the group consisting of carbon, nitrogen, oxygen and sulfur.

When the “monocyclic ring” or the “fused ring” has a substituent, the substituent is the substituent described in later-described “optional substituent.” When the “monocyclic ring” or the “fused ring” has a substituent, specific examples of the substituent are the substituents described in the above under the subtitle “Substituents Mentioned Herein.”

When the “saturated ring” or the “unsaturated ring” has a substituent, the substituent is, for instance, the substituent described in later-described “optional substituent.” When the “monocyclic ring” or the “fused ring” has a substituent, specific examples of the substituent are the substituents described in the above under the subtitle “Substituents Mentioned Herein.”

The above is the description for the instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (sometimes referred to as an instance “bonded to form a ring”.

Substituent for Substituted or Unsubstituted Group

In an exemplary embodiment herein, the substituent for the substituted or unsubstituted group (sometimes referred to as an “optional substituent”) is selected from the group consisting of, for instance, an unsubstituted alkyl group having 1 to 50 carbon atoms, an unsubstituted alkenyl group having 2 to 50 carbon atoms, an unsubstituted alkynyl group having 2 to 50 carbon atoms, an unsubstituted cycloalkyl group having 3 to 50 ring carbon atoms, —Si(R₉₀₁)(R₉₀₂)(R₉₀₃), —O—(R₉₀₄), —S—(R₉₀₅), —N(R₉₀₆)(R₉₀₇), a halogen atom, a cyano group, a nitro group, an unsubstituted aryl group having 6 to 50 ring carbon atoms, and an unsubstituted heterocyclic group having 5 to 50 ring atoms.

Herein, R₉₀₁ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

When two or more R₉₀₁ are present, the two or more R₉₀₁ are mutually the same or different.

When two or more R₉₀₂ are present, the two or more R₉₀₂ are mutually the same or different.

When two or more R₉₀₃ are present, the two or more R₉₀₃ are mutually the same or different.

When two or more R₉₀₄ are present, the two or more R₉₀₄ are mutually the same or different.

When two or more R₉₀₅ are present, the two or more R₉₀₅ are mutually the same or different.

When two or more R₉₀₆ are present, the two or more R₉₀₅ are mutually the same or different.

When two or more R₉₀₇ are present, the two or more R₉₀₇ are mutually the same or different.

In an exemplary embodiment, the substituent for “substituted or unsubstituted” group is selected from the group consisting of an alkyl group having 1 to 50 carbon atoms, an aryl group having 6 to 50 ring carbon atoms, and a heterocyclic group having 5 to 50 ring atoms.

In an exemplary embodiment, the substituent for “substituted or unsubstituted” group is selected from the group consisting of an alkyl group having 1 to 18 carbon atoms, an aryl group having 6 to 18 ring carbon atoms, and a heterocyclic group having 5 to 18 ring atoms.

Specific examples of the above optional substituent are the same as the specific examples of the substituent described in the above under the subtitle “Substituent Mentioned Herein.”

Unless otherwise specified herein, adjacent ones of the optional substituents may form a “saturated ring” or an “unsaturated ring,” preferably a substituted or unsubstituted saturated five-membered ring, a substituted or unsubstituted saturated six-membered ring, a substituted or unsubstituted saturated five-membered ring, or a substituted or unsubstituted unsaturated six-membered ring, more preferably a benzene ring.

Unless otherwise specified herein, the optional substituent may further include a substituent. Examples of the substituent for the optional substituent are the same as the examples of the optional substituent.

Herein, numerical ranges represented by “AA to BB” represents a range whose lower limit is the value (AA) recited before “to” and whose upper limit is the value (BB) recited after “to.”

### First Exemplary Embodiment

Compound

A compound according to a first exemplary embodiment is a compound represented by a formula (1000B) below and having at least one group represented by a formula (110) below.

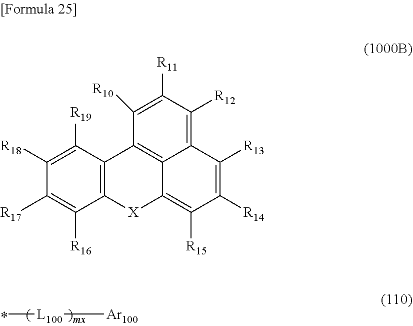

In the formula (1000B):
X is an oxygen atom, a sulfur atom, C(R₂₀₀₁)(R₂₀₀₂), or Si(R₂₀₀₃)(R₂₀₀₄);
at least one combination of adjacent two or more of R₁₀ to R₁₉ are mutually bonded to form a substituted or unsubstituted monocyclic ring, mutually bonded to form a substituted or unsubstituted fused ring, or not mutually bonded;
R₁₀ to R₁₉ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a nitro group, a substituted or unsubstituted aryl group having 6 to 50 ring carbon atoms, a substituted or unsubstituted heterocyclic group having 5 to 50 ring atoms, or a group represented by the formula (110);
at least one of R₁₀ to R₁₉ is a group represented by the formula (110);
when a plurality of groups represented by the formula (110) are present, the plurality of groups represented by the formula (110) are mutually the same or different;
L₁₀₀ is a single bond, a substituted or unsubstituted arylene group having 6 to 50 ring carbon atoms, or a substituted or unsubstituted divalent heterocyclic group having 5 to 50 ring atoms;
mx is 1, 2, or 3;
when two or more L₁₀₀ are present, the two or more L₁₀₀ are mutually the same or different;
Ar₁₀₀ is a substituted or unsubstituted aryl group including three or more rings or a substituted or unsubstituted heterocyclic group including two or more aromatic rings and one or more heterocyclic rings;
Ar₁₀₀ does not include an anthracene ring;
when two or more Ar₁₀₀ are present, the two or more Ar₁₀₀ are mutually the same or different; and
* in the formula (110) represents a bonding position.

A compound represented by the formula (1000B) is preferably a compound represented by a formula (100) below and having at least one group represented by the formula (110).

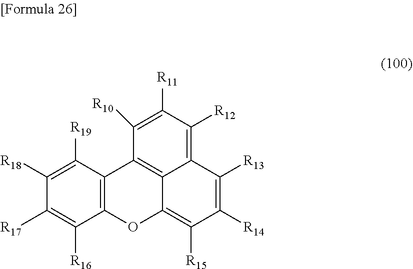

In the formula (100): R₁₀ to R₁₉ each independently represent the same as R₁₀ to R₁₉ in the formula (1000B); none of combinations of adjacent two or more of R₁₀ to R₁₉ are mutually bonded; and Ar₁₀₀, L₁₀₀, and mx respectively represent the same as Ar₁₀₀, L₁₀₀, and mx in the formula (110).

LUMO (Lowest Unoccupied Molecular Orbital) of a compound in which a cyano group is directly bonded to a benzoxanthene ring (hereinafter, referred to as a cyano-substituted benzoxanthene compound) is lower than LUMO of a compound in which a cyano group is not bonded to a benzoxanthene ring. In a case where a cyano-substituted benzoxanthene compound is used as a host material in the emitting layer, the host material may suffer a large damage at an interface of the emitting layer close to the hole transporting layer to lower a lifetime of an organic EL device. It is inappropriate to use a cyano-substituted benzoxanthene compound particularly in the emitting layer close to the hole transporting layer in an organic EL device in which a plurality of emitting layers are layered.

The compound according to the exemplary embodiment is also preferably represented by a formula (101) or (102) below.

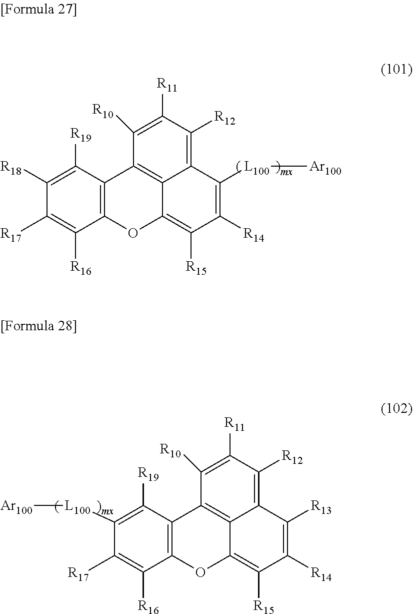

In the formulae (101) and (102): R₁₀ to R₁₉ each independently represent the same as R₁₀ to R₁₉ in the formula (1000B); none of combinations of adjacent two or more of R₁₀ to R₁₉ are mutually bonded; and Ar₁₀₀, L₁₀₀, and mx respectively represent the same as Ar₁₀₀, L₁₀₀, and mx in the formula (110).

In the compound according to the exemplary embodiment, it is preferable that R₁₀ to R₁₉ not being a group represented by the formula (110) are each independently a hydrogen atom, a substituted or unsubstituted alkyl group having 1 to 50 carbon atoms, a substituted or unsubstituted cycloalkyl group having 3 to 50 ring carbon atoms, or a substituted or unsubstituted aryl group having 6 to 50 ring carbon atoms.

In the compound according to the exemplary embodiment, R₁₀ to R₁₉ not being a group represented by the formula (110) are each preferably a hydrogen atom.

In the compound according to the exemplary embodiment, L₁₀₀ is preferably a single bond or an arylene group including at most three substituted or unsubstituted benzene rings.

In the compound according to the exemplary embodiment, L₁₀₀ is preferably not a substituted or unsubstituted anthrylene group.

In the compound according to the exemplary embodiment, L₁₀₀ is also preferably not a single bond.

In the compound according to the exemplary embodiment, a group represented by -(L₁₀₀)ₘₓ- in the formula (110) is also preferably a group represented by one of formulae (111) to (120) below.

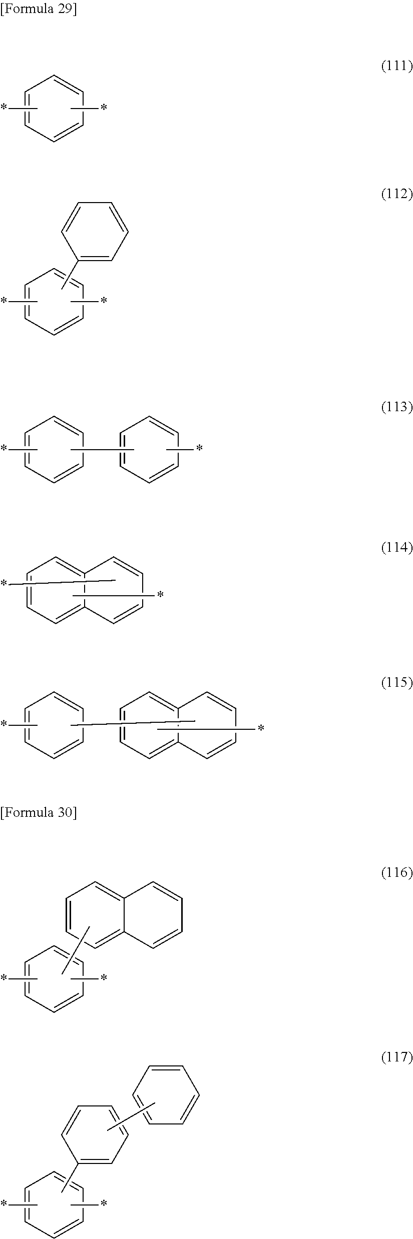

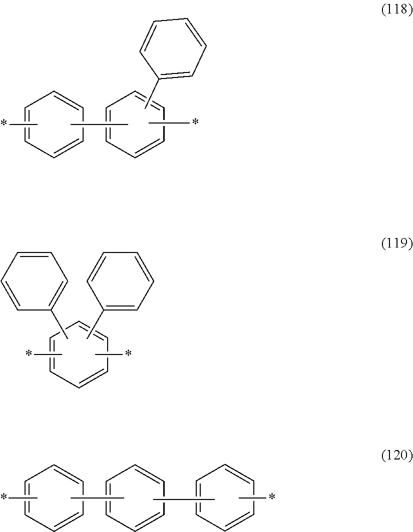

* in the formulae (111) to (120) represents a bonding position.

A group represented by -(L₁₀₀)ₘₓ- in the formula (110) is preferably a group represented by the formula (111) or (112).

In the compound according to the exemplary embodiment, Ar₁₀₀ is preferably an aryl group in which at least four substituted or unsubstituted benzene rings are fused.

In the compound according to the exemplary embodiment, Ar₁₀₀ is preferably an aryl group in which four substituted or unsubstituted benzene rings are fused or an aryl group in which five substituted or unsubstituted benzene rings are fused.

In the compound according to the exemplary embodiment, Ar₁₀₀ is preferably a group represented by a formula (1100), (1200), (1300), (1400), (1500), (1600), (1700), or (1800).

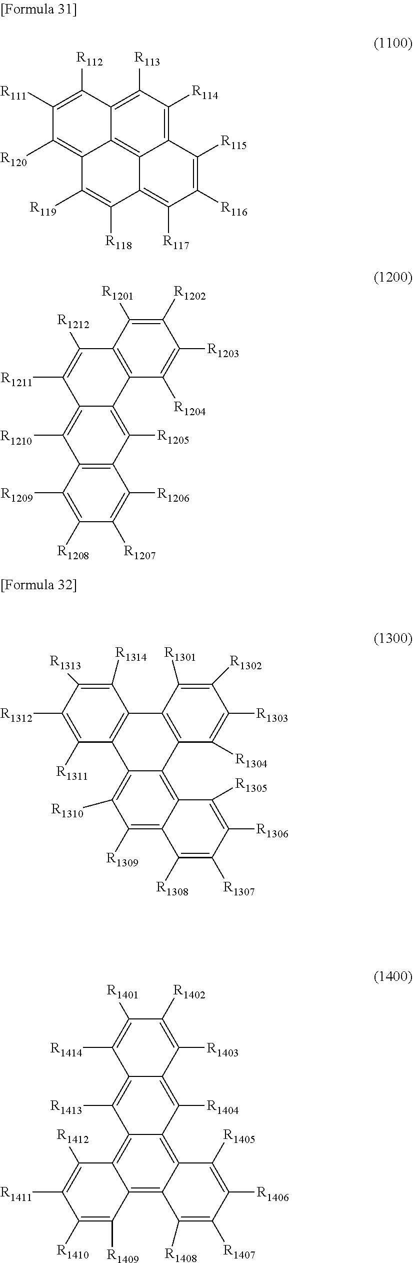

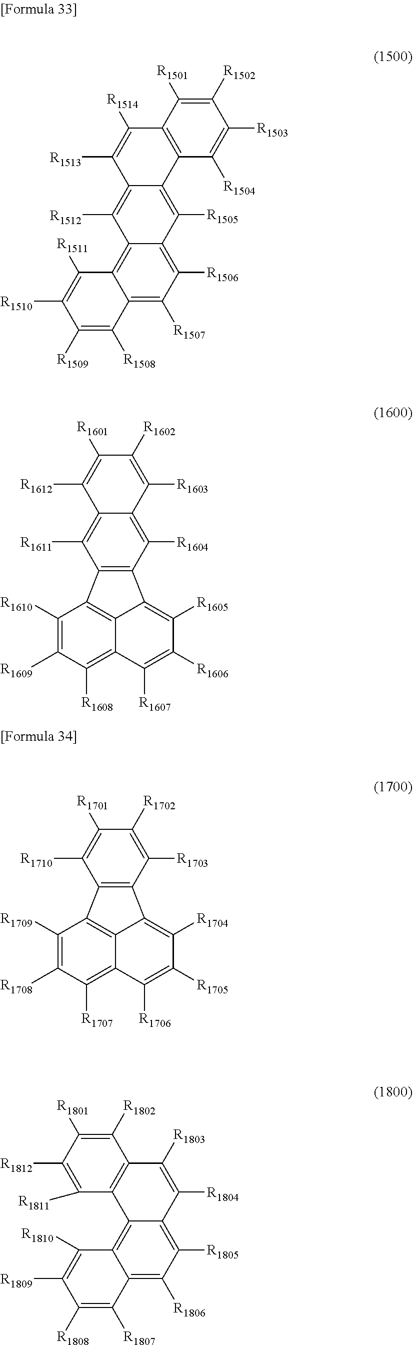

In the formula (1100), one of R₁₁ to R₁₂₀ is a bond.

In the formula (1200), one of R₁₂₀₁ to R₁₂₁₂ is a bond.

In the formula (1300), one of R₁₃₀₁ to R₁₃₁₄ is a bond.

In the formula (1400), one of R₁₄₀₁ to R₁₄₁₄ is a bond.

In the formula (1500), one of R₁₅₀₁ to R₁₅₁₄ is a bond.

In the formula (1600), one of R₁₆₀₁ to R₁₆₁₂ is a bond.

In the formula (1700), one of R₁₇₀₁ to R₁₇₁₀ is a bond.

In the formula (1800), one of R₁₈₀₁ to R₁₈₁₂ is a bond.

R₁₁₁ to R₁₂₀ not being a bond, R₁₂₀₁ to R₁₂₁₂ not being a bond, R₁₃₀₁ to R₁₃₁₄ not being a bond, R₁₄₀₁ to R₁₄₁₄ not being a bond, R₁₅₀₁ to R₁₅₁₄ not being a bond, R₁₆₀₁ to R₁₆₁₂ not being a bond, R₁₇₀₁ to R₁₇₁₀ not being a bond, and R₁₈₀₁ to R₁₈₁₂ not being a bond are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a nitro group, a substituted or unsubstituted aryl group having 6 to 50 ring carbon atoms, or a substituted or unsubstituted heterocyclic group having 5 to 50 ring atoms.

A group represented by the formula (1100) in which R₁₁₁ is a bond is represented by a formula (1112) below. A group represented by the formula (1100) in which R₁₂₀ is a bond is represented by a formula (1113) below. A group represented by the formula (1100) in which R₁₁₉ is a bond is represented by a formula (1114) below.

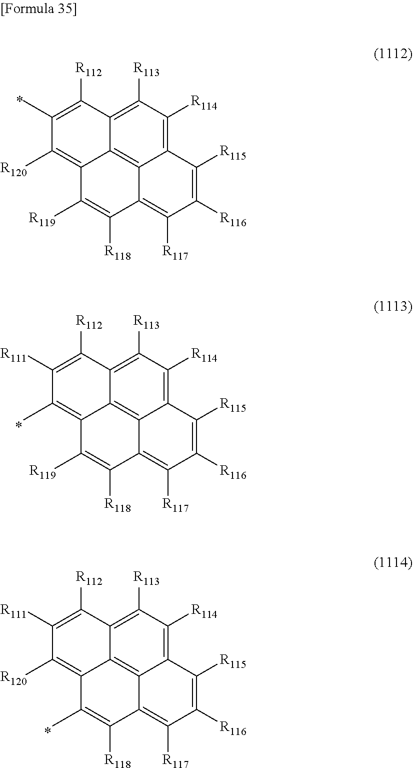

In the formulae (1112), (1113), and (1114):

R₁₁₁ to R₁₂₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a nitro group, a substituted or unsubstituted aryl group having 6 to 50 ring carbon atoms, or a substituted or unsubstituted heterocyclic group having 5 to 50 ring atoms; and
* in the formulae (1112) to (1114) represents a bonding position.

In the compound according to the exemplary embodiment, R₁₁₁ to R₁₂₀ not being a bond, R₁₂₀₁ to R₁₂₁₂ not being a bond, R₁₃₀₁ to R₁₃₁₄ not being a bond, R₁₄₀₁ to R₁₄₁₄ not being a bond, R₁₅₀₁ to R₁₅₁₄ not being a bond, R₁₆₀₁ to R₁₆₁₂ not being a bond, R₁₇₀₁ to R₁₇₁₀ not being a bond, and R₁₈₀₁ to R₁₈₁₂ not being a bond are preferably each independently a hydrogen atom, a substituted or unsubstituted alkyl group having 1 to 50 carbon atoms, a substituted or unsubstituted cycloalkyl group having 3 to 50 ring carbon atoms, or a substituted or unsubstituted aryl group having 6 to 50 ring carbon atoms.

In the compound according to the exemplary embodiment, R₁₁₁ to R₁₂₀ not being a bond, R₁₂₀₁ to R₁₂₁₂ not being a bond, R₁₃₀₁ to R₁₃₁₄ not being a bond, R₁₄₀₁ to R₁₄₁₄ not being a bond, R₁₅₀₁ to R₁₅₁₄ not being a bond, R₁₆₀₁ to R₁₆₁₂ not being a bond, R₁₇₀₁ to R₁₇₁₀ not being a bond, and R₁₈₀₁ to R₁₈₁₂ not being a bond are preferably each a hydrogen atom.

In the compound according to the exemplary embodiment, Ar₁₀₀ as a substituted or unsubstituted heterocyclic group including at least two aromatic rings and at least one heterocyclic group is also preferably, for instance, a substituted or unsubstituted benzoxanthenyl group, a substituted or unsubstituted dibenzofuranyl group, a substituted or unsubstituted naphthobenzofuranyl group, or a substituted or unsubstituted dibenzothienyl group.

The compound according to the exemplary embodiment preferably does not include a cyano group in a molecule.

The compound according to the exemplary embodiment also preferably includes one or two benzoxanthene rings in a molecule.

The compound according to the exemplary embodiment also preferably includes only a single benzoxanthene ring in a molecule.

The compound according to the exemplary embodiment is also preferably a compound having at least one group represented by a formula (11) below and a single benz[de]anthracene derivative skeleton represented by a formula (1000) below in a molecule.

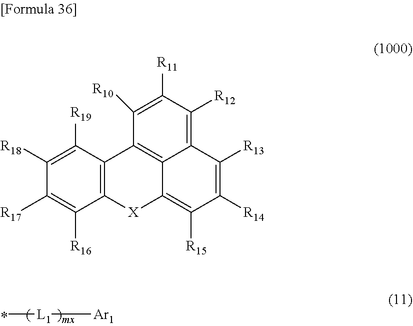

In the formula (1000):
X is an oxygen atom, a sulfur atom, C(R₂₀₀₁)(R₂₀₀₂), or Si(R₂₀₀₃)(R₂₀₀₄);
at least one combination of adjacent two or more of R₁₀ to R₁₉ are mutually bonded to form a substituted or unsubstituted monocyclic ring, mutually bonded to form a substituted or unsubstituted fused ring, or not mutually bonded;
R₁₀ to R₁₉ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a nitro group, a substituted or unsubstituted aryl group having 6 to 50 ring carbon atoms, a substituted or unsubstituted heterocyclic group having 5 to 50 ring atoms, or a group represented by the formula (11),
at least one of R₁₀ to R₁₉ is a group represented by the formula (11);
when a plurality of groups represented by the formula (11) are present, the plurality of groups represented by the formula (11) are mutually the same or different;
L₁ is a substituted or unsubstituted arylene group having 6 to 15 ring carbon atoms, or a substituted or unsubstituted divalent heterocyclic group having 5 to 15 ring atoms;
mx is 1, 2, or 3;
when two or more L₁ are present, the two or more L₁ are mutually the same or different;
Ar₁ is a substituted or unsubstituted aryl group including four or more rings;
when two or more Ar₁ are present, the two or more Ar₁ are mutually the same or different; and
* in the formula (11) represents a bonding position.

A compound represented by the formula (1000) preferably includes at least one group represented by the formula (11) and a single benzoxanthene ring represented by a formula (1) below in a molecule.

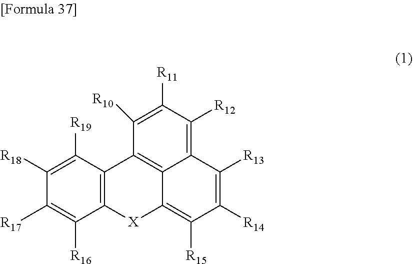

In the formula (1): R₁₀ to R₁₉ each independently represent the same as R₁₀ to R₁₉ in the formula (1000); none of combinations of adjacent two or more of R₁₀ to R₁₉ are mutually bonded; and Ar₁, L₁, and mx respectively represent the same as Ar₁, L₁, and mx in the formula (11).

In a compound represented by the formula (1), L₁ that is a substituted or unsubstituted arylene group having 6 to 15 ring carbon atoms or a substituted or unsubstituted divalent heterocyclic group having 5 to 15 ring atoms is provided between a benzoxanthene ring and Ar₁ that is a substituted or unsubstituted aryl group having four or more rings, whereby a decrease in a hole mobility can be inhibited and a decrease in a luminous efficiency of an organic EL device can be also inhibited.

The compound including at least one group represented by the formula (11) and a single benzoxanthene ring represented by the formula (1) in a molecule is preferably represented by a formula (121) or (122).

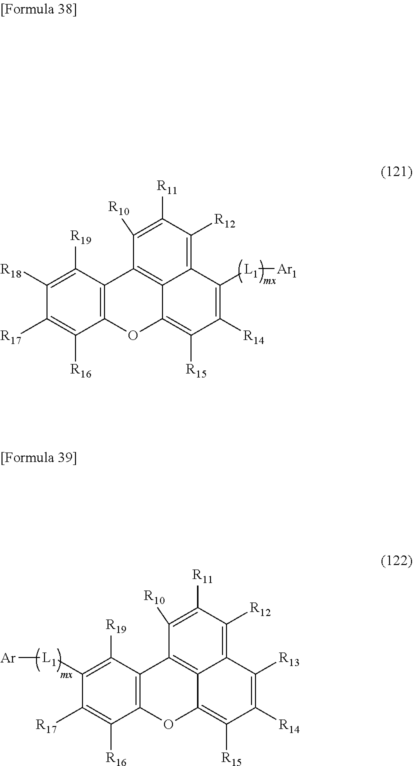

In the formulae (121) and (122): R₁₀ to R₁₉ each independently represent the same as R₁₀ to R₁₉ in the formula (1); and Ar₁, L₁, and mx respectively represent the same as Ar₁, L₁, and mx in the formula (11).

In the formula (121), R₁₀ to R₁₂ and R₁₄ to R₁₉ are each preferably not a group represented by the formula (11).

In the formula (122), R₁₀ to R₁₇ and R₁₉ are each preferably not a group represented by the formula (11).

In a compound represented by the formula (1), R₁₀ to R₁₉ not being a group represented by the formula (11) are preferably each independently a hydrogen atom, a substituted or unsubstituted alkyl group having 1 to 50 carbon atoms, a substituted or unsubstituted cycloalkyl group having 3 to 50 ring carbon atoms, or a substituted or unsubstituted aryl group having 6 to 50 ring carbon atoms.

In a compound represented by the formula (1), R₁₀ to R₁₉ not being a group represented by the formula (11) are preferably each a hydrogen atom.

In a compound represented by the formula (1), L₁ is preferably an arylene group including at most three substituted or unsubstituted benzene rings.

In a compound represented by the formula (1), L₁ is preferably not a substituted or unsubstituted anthrylene group.

In a compound represented by the formula (1), a group represented by -(L₁)ₘₓ- in the formula (11) is also preferably a group represented by one of the formulae (111) to (120).

In a compound represented by the formula (1), a group represented by -(L₁)mₓ- in the formula (11) is preferably a group represented by one of the formulae (111) and (112).

The compound including at least one group represented by the formula (11) and a single benz[de]anthracene derivative skeleton represented by the formula (1000) in a molecule is also preferably represented by a formula (123), (124), (125), or (126).

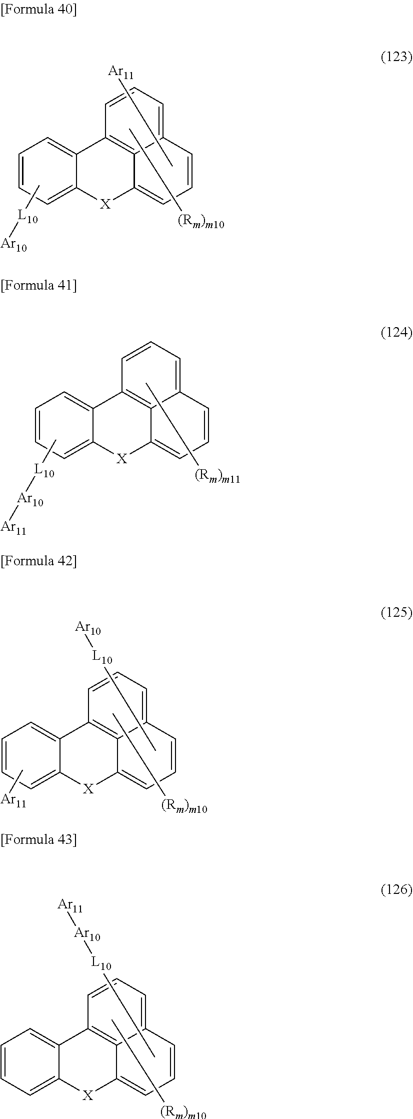

In the formulae (123), (124), (125), and (126):
X is an oxygen atom, a sulfur atom, C(R₂₀₀₁)(R₂₀₀₂), or Si(R₂₀₀₃)(R₂₀₀₄);
R₂₀₀₁ to R₂₀₀₄ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
L₁₀ is a substituted or unsubstituted arylene group having 6 to 15 ring carbon atoms, or a substituted or unsubstituted divalent heterocyclic group having 5 to 15 ring atoms;
Ar₁₀ is an aryl group in which at least four substituted or unsubstituted benzene rings are fused;
Ar₁₁ is a substituted or unsubstituted aryl group having 6 to 14 ring carbon atoms;
Ar₁₁ is not a substituted or unsubstituted anthryl group;
m10 is 5;
m11 is 6;
Rm is each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a nitro group, a substituted or unsubstituted aryl group having 6 to 50 ring carbon atoms, or a substituted or unsubstituted heterocyclic group having 5 to 50 ring atoms;
a plurality of Rm are not mutually bonded;
R₉₀₁, R₉₀₂, R₉₀₃, R₉₀₄, R₉₀₅, R₉₀₆, R₉₀₇, R₈₀₁, and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

As in a compound represented by the formula (123) and a compound represented by the formula (125), a benz[de]anthracene derivative skeleton is substituted by Ar₁₁ (a substituted or unsubstituted aryl group having 6 to 14 ring carbon atoms except for a substituted or unsubstituted anthryl group), whereby a singlet energy S₁ can be decreased and an excited state can be expected to be stabilized, so that a lifetime of an organic EL device can be prolonged.

As in a compound represented by the formula (124) and a compound represented by the formula (126), Ar₁₀ (an aryl group in which at least four substituted or unsubstituted benzene rings are fused) is substituted by Ar₁₁ (a substituted or unsubstituted aryl group having 6 to 14 ring carbon atoms except for a substituted or unsubstituted anthryl group), whereby electron transportability can be improved and a carrier balance of an organic EL device using the compound can be expected to be improved.

A compound represented by the formula (123) is more preferably represented by a formula (1230) below. A compound represented by the formula (124) is more preferably represented by a formula (1240) below. A compound represented by the formula (125) is more preferably represented by a formula (1250) below. A compound represented by the formula (126) is more preferably represented by a formula (1260) below.

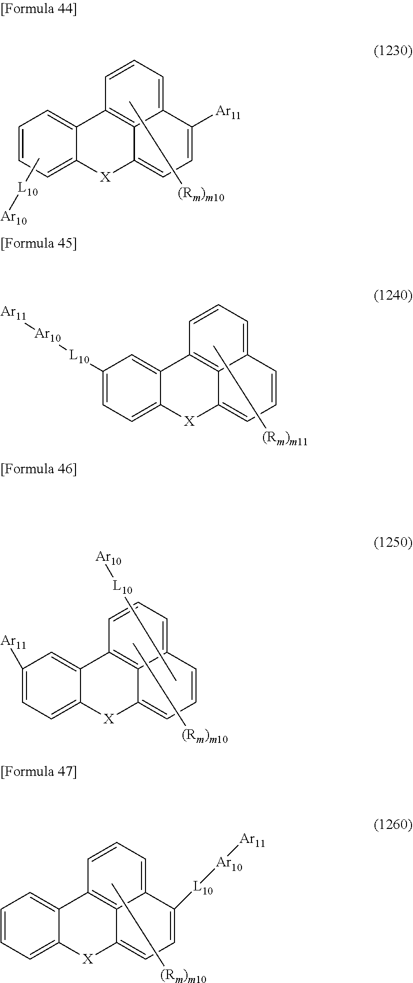

In the formulae (1230), (1240), (1250), and (1260), X, L₁₀, Ar₁₀, Ar₁₁, m10, m11, and Rm each independently represent the same as L₁₀, Ar₁₀, Ar₁₁, m10, m11, and Rm in the formulae (123), (124), (125), and (126).

In a compound represented by the formula (123), (124), (125), (126), (1230), (1240), (1250), or (1260), X is preferably an oxygen atom.

In a compound represented by the formula (123), (124), (125), (126), (1230), (1240), (1250), or (1260), L₁₀ is preferably an arylene group including at most three substituted or unsubstituted benzene rings.

In a compound represented by the formula (123), (124), (125), (126), (1230), (1240), (1250), or (1260), a group represented by -L₁₀- is preferably a group represented by one of the formulae (111) to (120).

In a compound represented by the formula (123), (124), (125), (126), (1230), (1240), (1250), or (1260), a group represented by -L₁₀- is preferably a group represented by the formula (111) or (112).

The compound according to the exemplary embodiment is also preferably a compound having at least one group represented by a formula (110A) below and a single benz[de]anthracene derivative skeleton represented by a formula (1000A) below in a molecule.

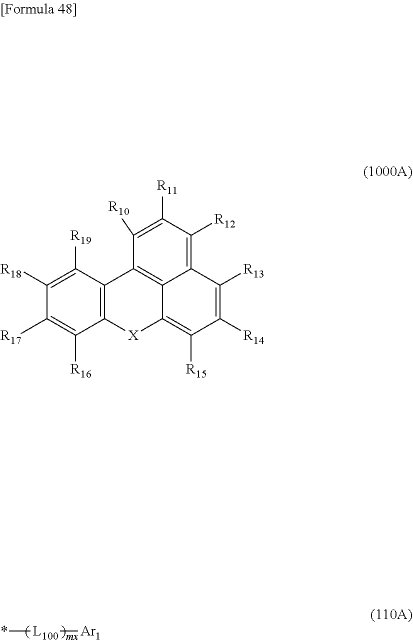

In the formula (1000A):
X is an oxygen atom, a sulfur atom, C(R₂₀₀₁)(R₂₀₀₂), or Si(R₂₀₀₃)(R₂₀₀₄);
at least one combination of adjacent two or more of R₁₀ to R₁₉ are mutually bonded to form a substituted or unsubstituted monocyclic ring, mutually bonded to form a substituted or unsubstituted fused ring, or not mutually bonded;
R₁₀ to R₁₉ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a nitro group, a substituted or unsubstituted aryl group having 6 to 50 ring carbon atoms, a substituted or unsubstituted heterocyclic group having 5 to 50 ring atoms, or a group represented by the formula (110A);
at least one of R₁₃ or R₁₈ is a group represented by the formula (110A);
when a plurality of groups represented by the formula (110A) are present, the plurality of groups represented by the formula (110A) are mutually the same or different;
L₁₀₀ is a single bond, a substituted or unsubstituted arylene group having 6 to 50 ring carbon atoms, or a substituted or unsubstituted divalent heterocyclic group having 5 to 50 ring atoms;
mx is 1, 2, or 3;
when two or more L₁₀₀ are present, the two or more L₁₀₀ are mutually the same or different;
Ar₁ is a substituted or unsubstituted aryl group including four or more rings;
when two or more Ar₁ are present, the two or more Ar₁ are mutually the same or different; and
* in the formula (110A) represents a bonding position.

A compound represented by the formula (1000A) preferably includes at least one group represented by the formula (110A) and a single benzoxanthene ring represented by a formula (100A) below in a molecule.

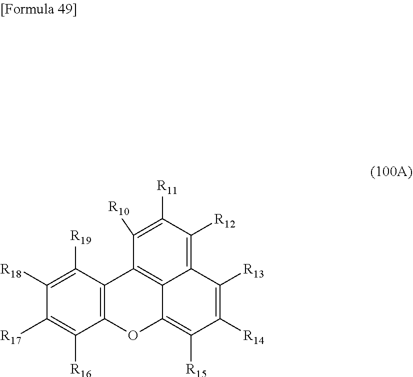

In the formula (100A): R₁₀ to R₁₉ each independently represent the same as R₁₀ to R₁₉ in the formula (1000A); none of combinations of adjacent two or more of R₁₀ to R₁₉ are mutually bonded; and Ar₁, L₁, and mx respectively represent the same as Ar₁, L₁, and mx in the formula (110A).

In a compound represented by the formula (100A), at least one of R₁₃ or R₁ is a group having Ar₁ that is a substituted or unsubstituted aryl group including four or more rings. Hole injectability of the compound according to the exemplary embodiment is improved by Ar₁ being thus bonded to a benzoxanthene ring at a para position with respect to an oxygen atom (O) through a single bond, a substituted or unsubstituted arylene group having 6 to 50 ring carbon atoms, or a substituted or unsubstituted divalent heterocyclic group having 5 to 50 ring atoms. Therefore, the luminous efficiency of the organic EL device containing the compound according to the exemplary embodiment can be expected to be improved.

A compound represented by the formula (100A) is also preferably represented by a formula (121A) or (122A) below.

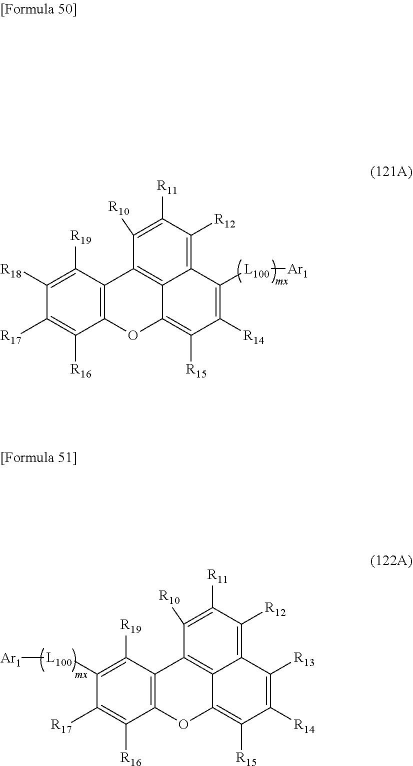

In the formulae (121A) and (122A): R₁₀ to R₁₉ each independently represent the same as R₁₀ to R₁₉ in the formula (100A); and Ar₁, L₁₀₀, and mx respectively represent the same as Ar₁, L₁₀₀, and mx in the formula (110A).

In the formula (121A), R₁₀ to R₁₂ and R₁₄ to R₁₉ are each preferably not a group represented by the formula (110A).

In the formula (122A), R₁₀ to R₁₇ and R₁₉ are each preferably not a group represented by the formula (110A).

In the compound represented by the formula (100A), R₁₀ to R₁₉ not being a group represented by the formula (110A) are preferably each independently a hydrogen atom, a substituted or unsubstituted alkyl group having 1 to 50 carbon atoms, a substituted or unsubstituted cycloalkyl group having 3 to 50 ring carbon atoms, or a substituted or unsubstituted aryl group having 6 to 50 ring carbon atoms.

In a compound represented by the formula (100A), R₁₀ to R₁₉ not being a group represented by the formula (110A) are preferably each a hydrogen atom.

In a compound represented by the formula (100A), L₁₀₀ is preferably a single bond or an arylene group including at most three substituted or unsubstituted benzene rings.

In a compound represented by the formula (100A), a group represented by -(L₁₀₀)ₘₓ- is preferably a group represented by one of the formulae (111) to (120).

In a compound represented by the formula (100A), a group represented by -(L₁₀₀)ₘₓ- is preferably a group represented by the formula (111) or (112).

In the compound according to the exemplary embodiment, Ar₁ is preferably an aryl group in which at least four substituted or unsubstituted benzene rings are fused.

In the compound according to the exemplary embodiment, Ar₁ or Ar₁₀ is preferably a substituted or unsubstituted aryl group in which four benzene rings are fused or a substituted or unsubstituted aryl group in which five benzene rings are fused.

In the compound according to the exemplary embodiment, Ar₁ or Ar₁₀ is preferably a group represented by a formula (1100), (1200), (1300), (1400), (1500), (1600), (1700), or (1800).

In the compound according to the exemplary embodiment, Ar₁ or Ar₁₀ is preferably a group represented by the formula (1100) or (1200).

In the compound according to the exemplary embodiment, it is preferable that all of the “substituted or unsubstituted” groups are “unsubstituted” groups.

In the compound according to the exemplary embodiment (e.g., a compound represented by the formula (1000B), a compound represented by the formula (1000), a compound represented by the formula (1000A)), in a case of “at least one of combinations of adjacent two or more of R₁₀ to R₁₉ are mutually bonded to form a ring (substituted or unsubstituted monocyclic ring or substituted or unsubstituted fused ring), examples of the combinations of adjacent two or more include a combination of R₁₀ and R₁₁, a combination of R₁₁ and R₁₂, a combination of R₁₂ and R₁₃, a combination of R₁₃ and R₁₄, a combination of R₁₄ and R₁₅, a combination of R₁₆ and R₁₇, a combination of R₁₇ and R₁₈, a combination of R₁₈ and R₁₉, and a combination of R₁₉ and R₁₀.

In the compound according to the exemplary embodiment (e.g., a compound represented by the formula (1000B), a compound represented by the formula (1000), a compound represented by the formula (1000A)), it is preferable that a combination of R₁₃ and R₁₄, a combination of R₁₆ and R₁₇, or a combination of R₁₉ and R₁₀ are mutually bonded to form a substituted or unsubstituted benzene ring.

In the compound according to the exemplary embodiment (e.g., a compound represented by the formula (1000B), a compound represented by the formula (1000), a compound represented by the formula (1000A)), R₂₀₀₁ to R₂₀₀₄ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In the compound according to the exemplary embodiment (e.g., a compound represented by the formula (1000B), a compound represented by the formula (1000), a compound represented by the formula (1000A)), R₂₀₀₁ to R₂₀₀₄ are preferably each independently a hydrogen atom, a substituted or unsubstituted alkyl group having 1 to 50 carbon atoms, a halogen atom, a cyano group, a nitro group, a substituted or unsubstituted aryl group having 6 to 50 ring carbon atoms, or a substituted or unsubstituted heterocyclic group having 5 to 50 ring atoms.

In the compound according to the exemplary embodiment (e.g., a compound represented by the formula (1000B), a compound represented by the formula (1000), a compound represented by the formula (1000A)):
R₉₀₁, R₉₀₂, R₉₀₃, R₉₀₄, R₉₀₅, R₉₀₆, R₉₀₇, R₈₀₁, and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

A hydrogen atom in the compound according to the exemplary embodiment is a protium atom, a deuterium atom, or a tritium atom. In an exemplary embodiment, a hydrogen atom in the compound according to the exemplary embodiment is a protium atom. In an exemplary embodiment, a hydrogen atom in the compound according to the exemplary embodiment is a deuterium atom.

Manufacturing Method of Compound According to First Exemplary Embodiment

The compound according to the exemplary embodiment can be manufactured by application of known substitution reactions and materials depending on a target compound, in accordance with or based on synthesis methods described later in Examples.

### Specific Examples of Compound According to First Exemplary Embodiment

Examples of the compound according to the exemplary embodiment include the following compounds. However, the invention is not limited to these specific examples. In the chemical formulae herein, a deuterium atom is denoted by D and a protium atom is denoted by H or a description for a protium is omitted.

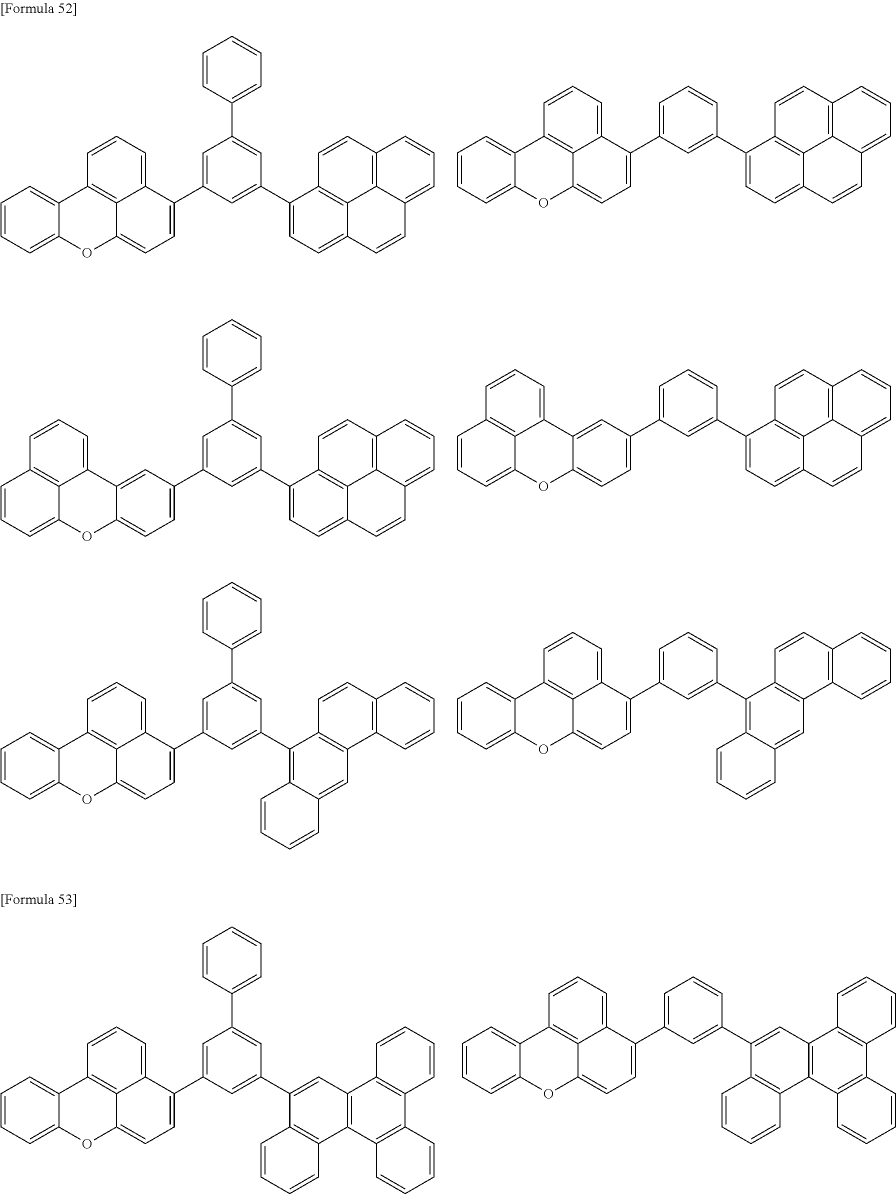

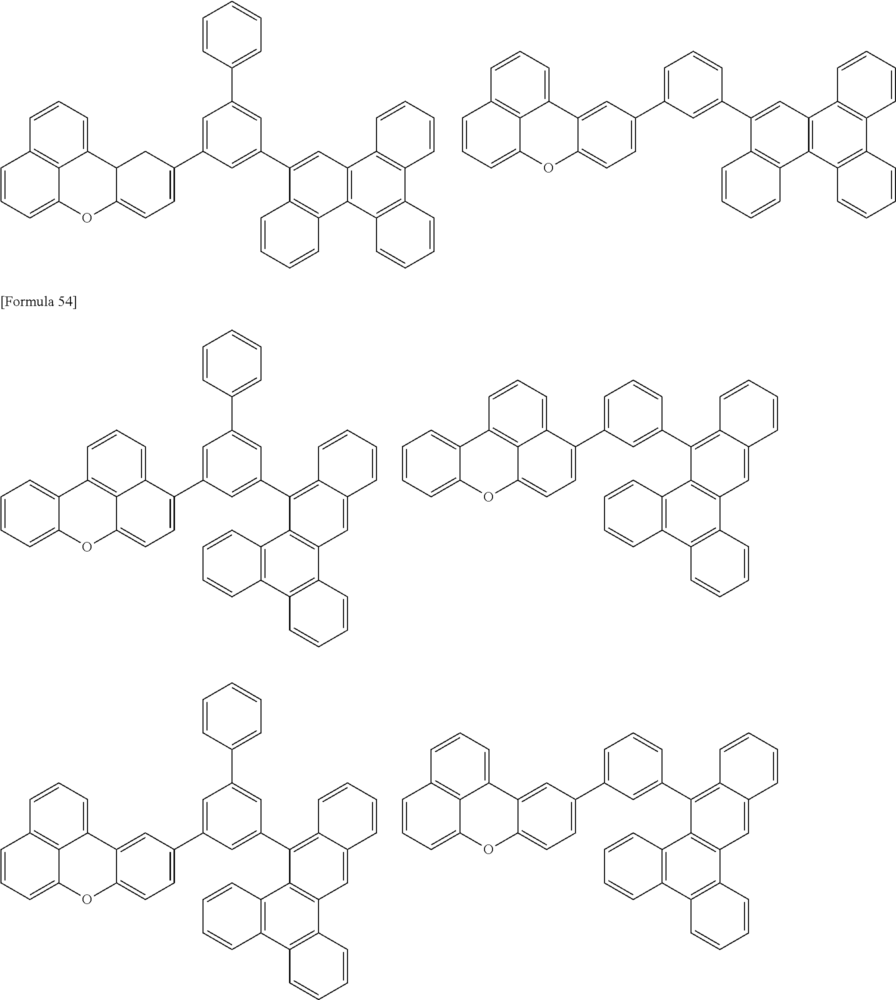

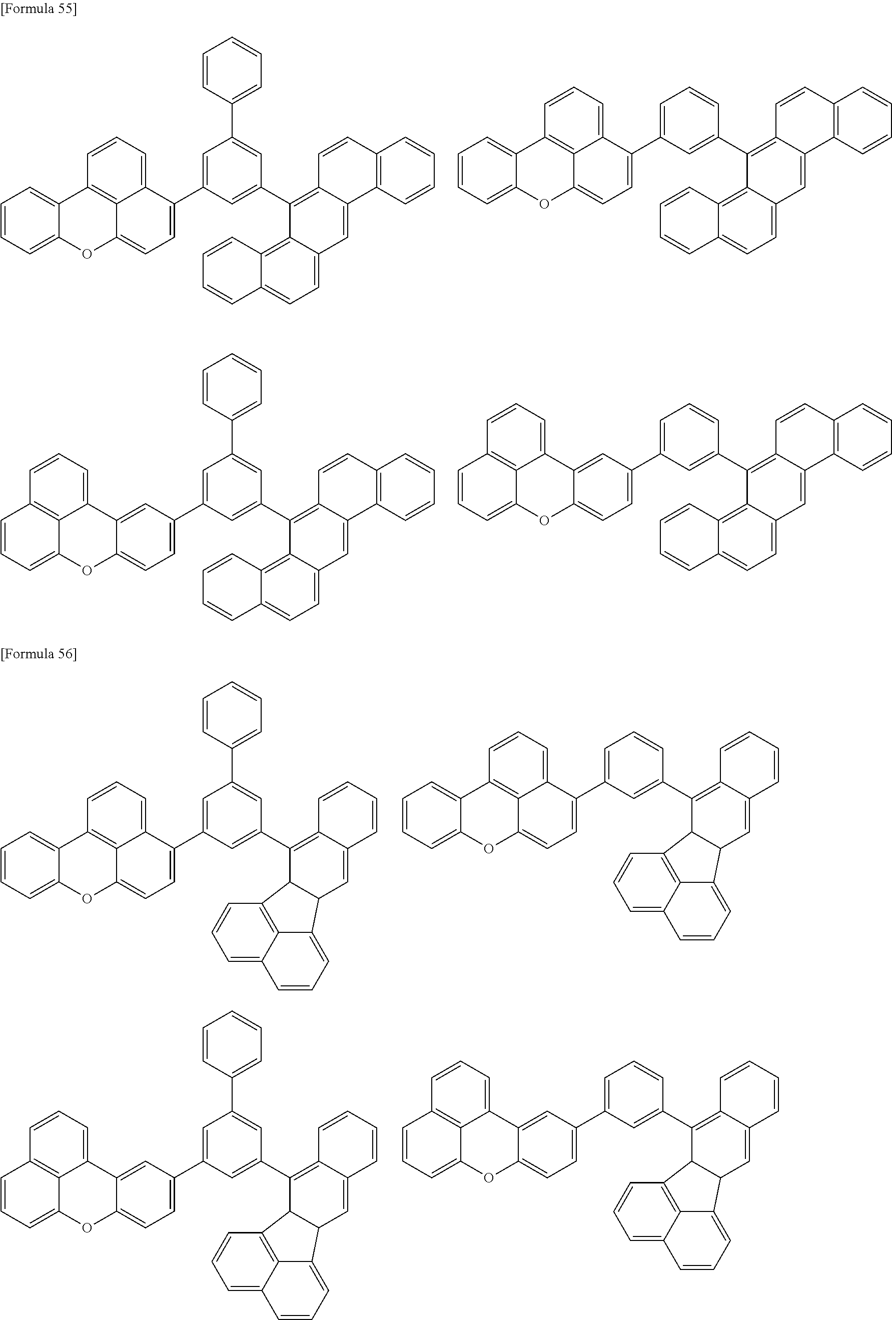

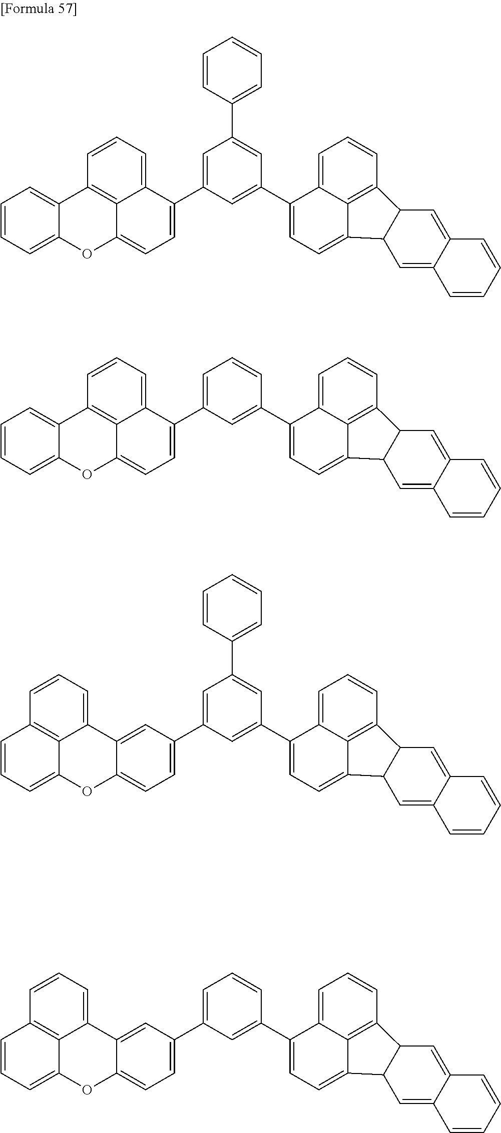 [SMILES: C1=c2ccccc2=CC2c3ccc(-c4cc(-c5ccccc5)cc(-c5ccc6c7c(cccc57)-c5ccccc5O6)c4)c4cccc(c34)C12 ; C1=c2ccccc2=CC2c3ccc(-c4cccc(-c5ccc6c7c(cccc57)-c5ccccc5O6)c4)c4cccc(c34)C12 ; C1=c2ccccc2=CC2c3ccc(-c4cc(-c5ccccc5)cc(-c5ccc6c(c5)-c5cccc7cccc(c57)O6)c4)c4cccc(c34)C12 ; C1=c2ccccc2=CC2c3ccc(-c4cccc(-c5ccc6c(c5)-c5cccc7cccc(c57)O6)c4)c4cccc(c34)C12]

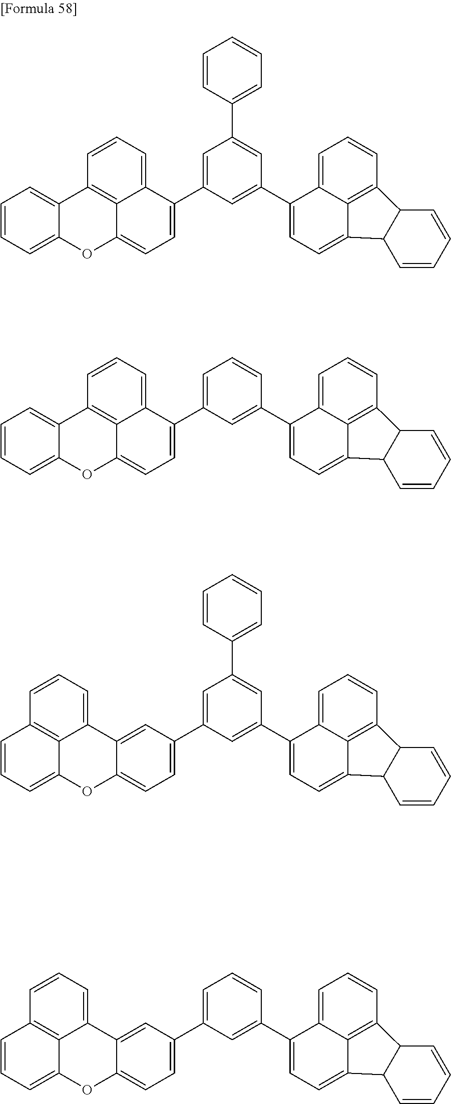 [SMILES: C1=CC2c3cccc4c(-c5cc(-c6ccccc6)cc(-c6ccc7c8c(cccc68)-c6ccccc6O7)c5)ccc(c34)C2C=C1 ; C1=CC2c3cccc4c(-c5cccc(-c6ccc7c8c(cccc68)-c6ccccc6O7)c5)ccc(c34)C2C=C1 ; C1=CC2c3cccc4c(-c5cc(-c6ccccc6)cc(-c6ccc7c(c6)-c6cccc8cccc(c68)O7)c5)ccc(c34)C2C=C1 ; C1=CC2c3cccc4c(-c5cccc(-c6ccc7c(c6)-c6cccc8cccc(c68)O7)c5)ccc(c34)C2C=C1]

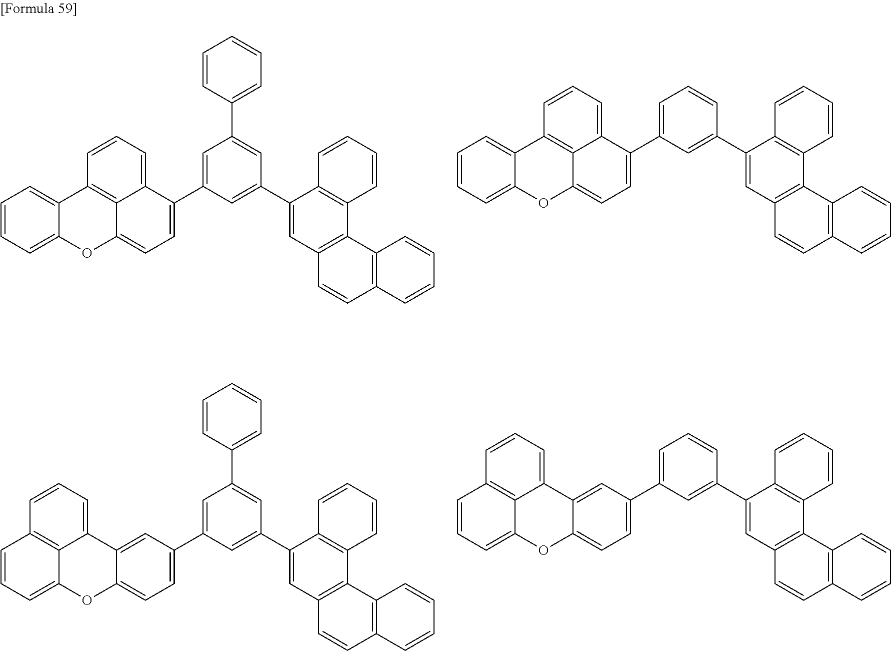 [SMILES: c1ccc(-c2cc(-c3ccc4c5c(cccc35)-c3ccccc3O4)cc(-c3cc4ccc5ccccc5c4c4ccccc34)c2)cc1 ; c1cc(-c2ccc3c4c(cccc24)-c2ccccc2O3)cc(-c2cc3ccc4ccccc4c3c3ccccc23)c1 ; c1ccc(-c2cc(-c3ccc4c(c3)-c3cccc5cccc(c35)O4)cc(-c3cc4ccc5ccccc5c4c4ccccc34)c2)cc1 ; c1cc(-c2ccc3c(c2)-c2cccc4cccc(c24)O3)cc(-c2cc3ccc4ccccc4c3c3ccccc23)c1]

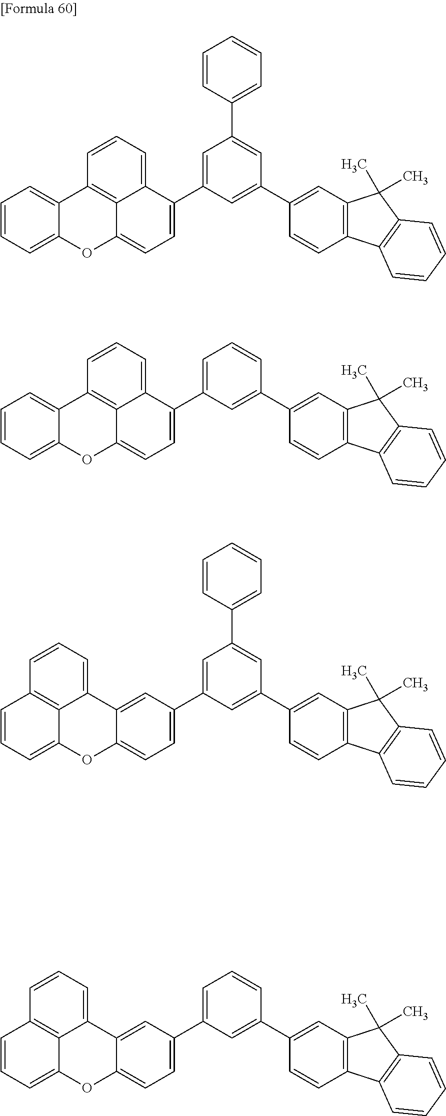 [SMILES: CC1(C)c2ccccc2-c2ccc(-c3cc(-c4ccccc4)cc(-c4ccc5c6c(cccc46)-c4ccccc4O5)c3)cc21 ; CC1(C)c2ccccc2-c2ccc(-c3cccc(-c4ccc5c6c(cccc46)-c4ccccc4O5)c3)cc21 ; CC1(C)c2ccccc2-c2ccc(-c3cc(-c4ccccc4)cc(-c4ccc5c(c4)-c4cccc6cccc(c46)O5)c3)cc21 ; CC1(C)c2ccccc2-c2ccc(-c3cccc(-c4ccc5c(c4)-c4cccc6cccc(c46)O5)c3)cc21]

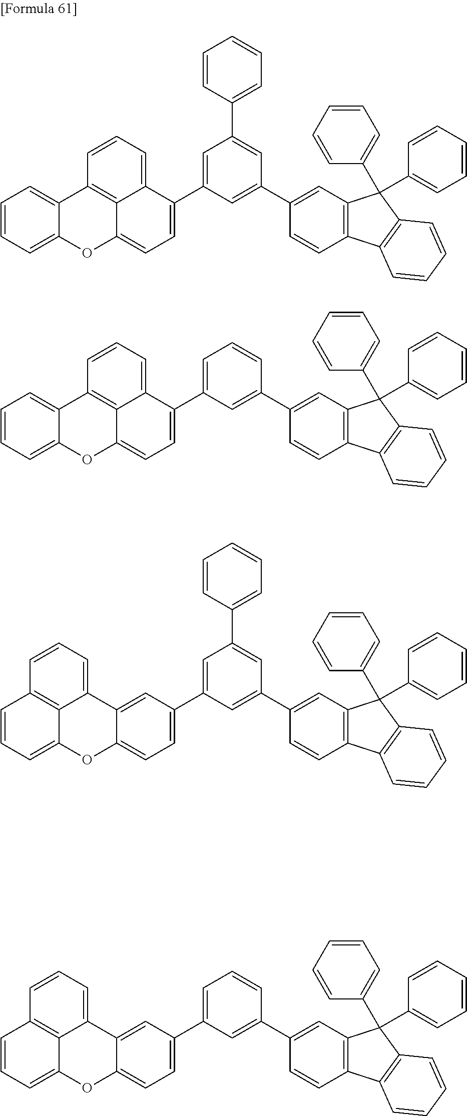

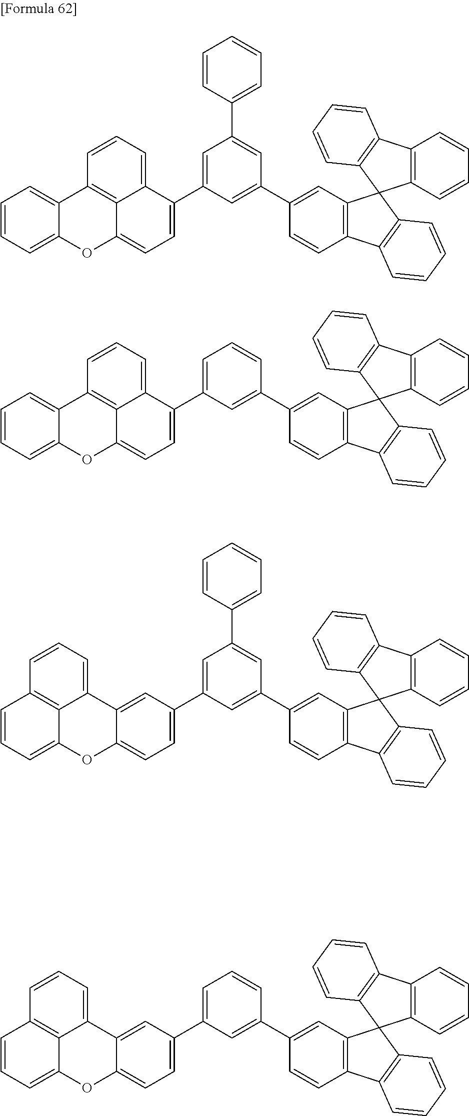

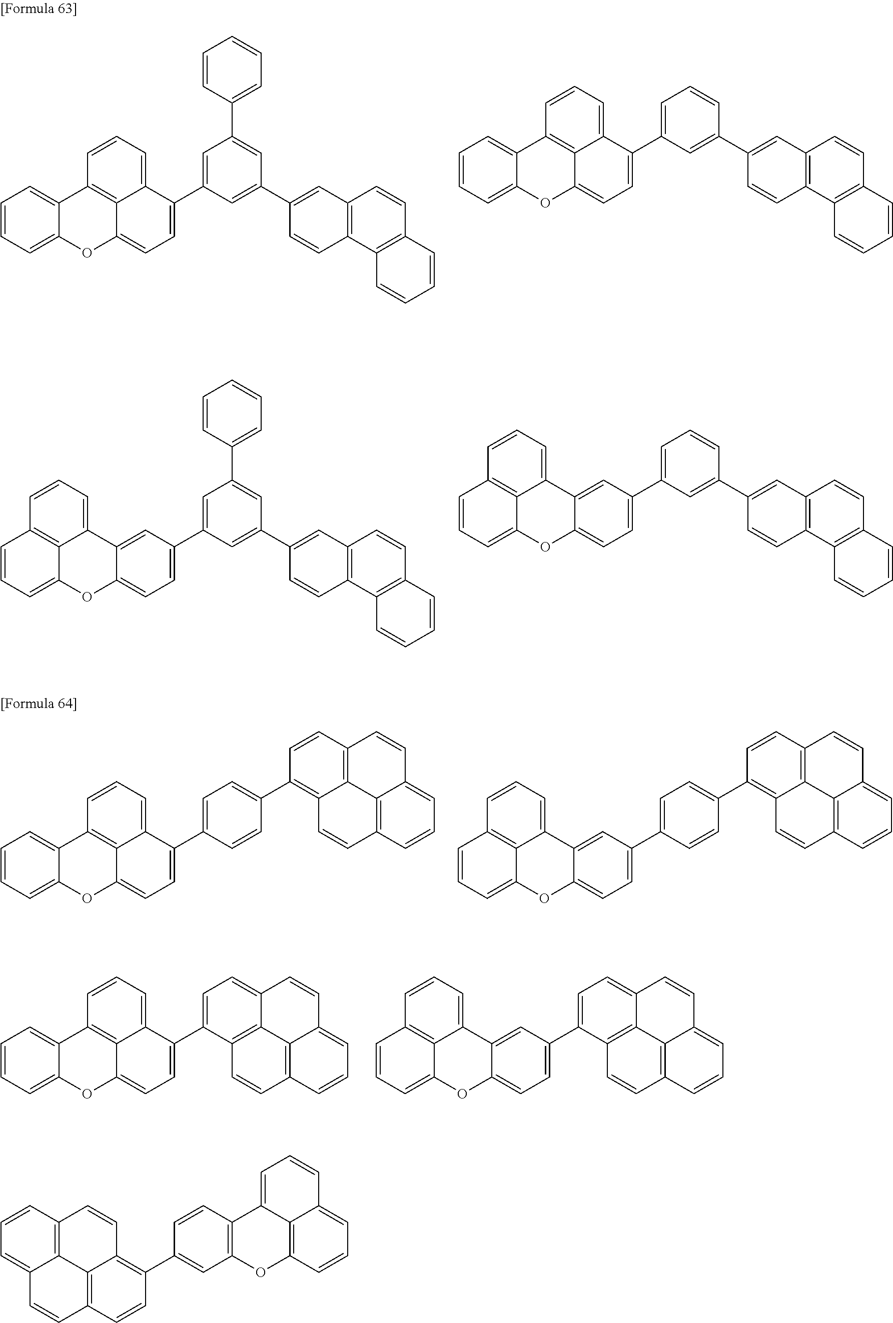

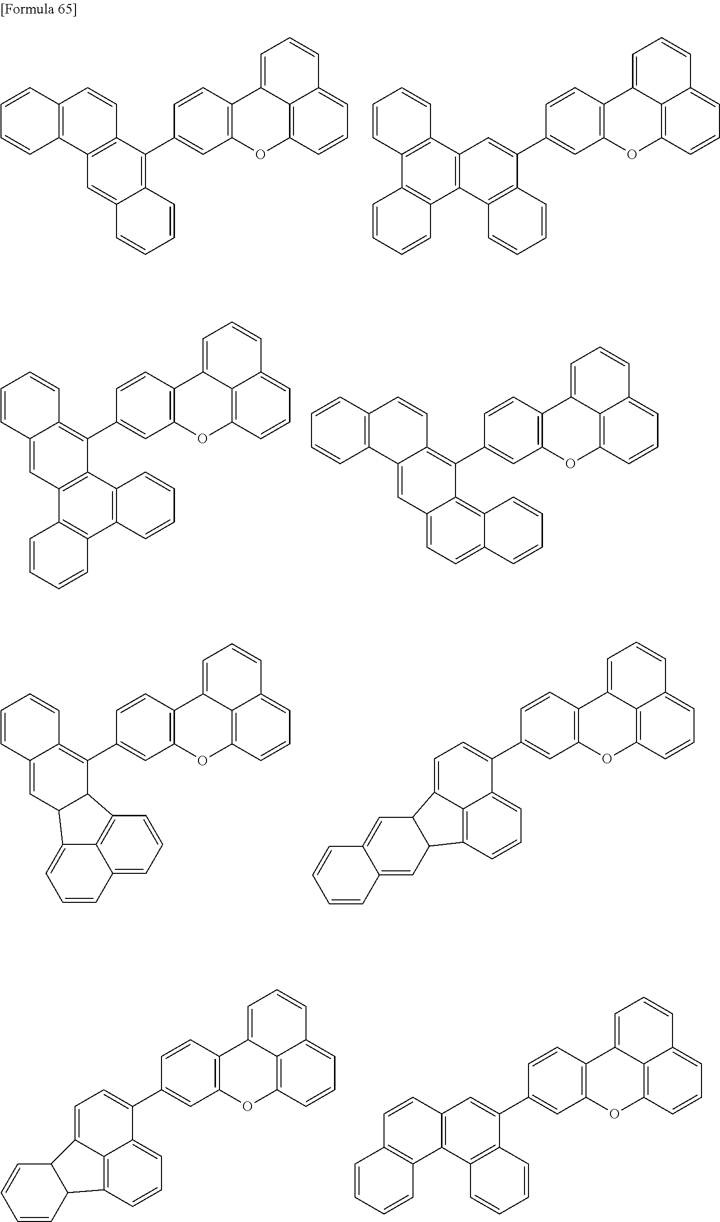

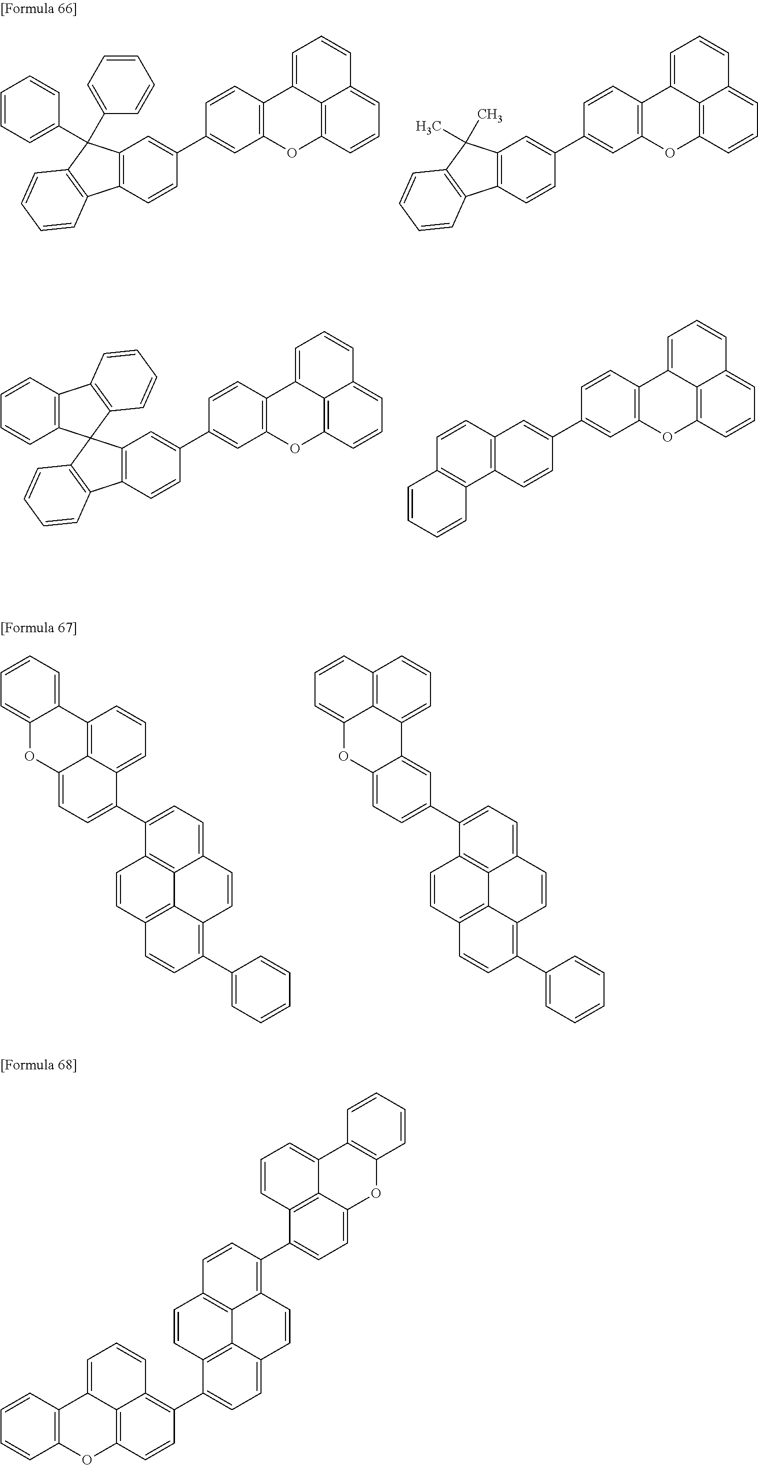

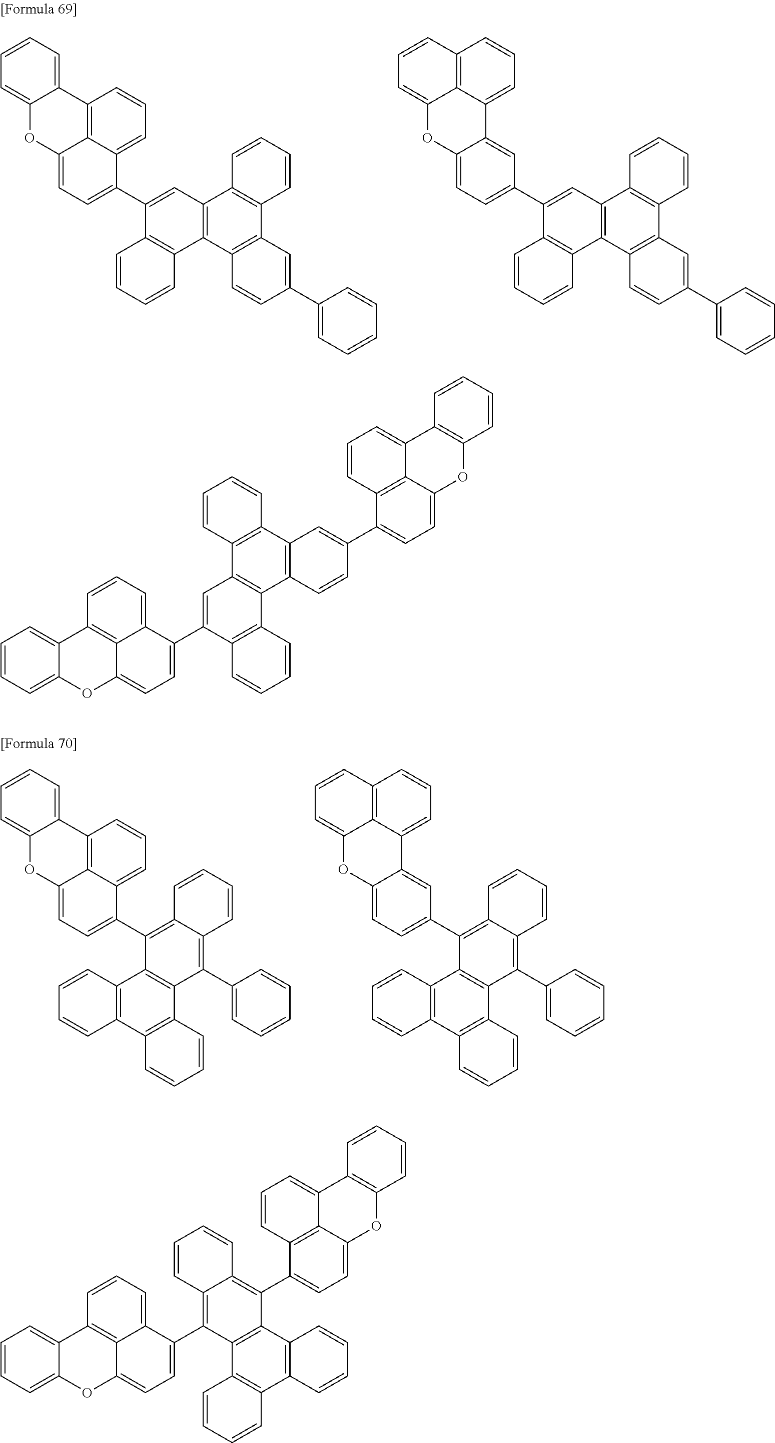

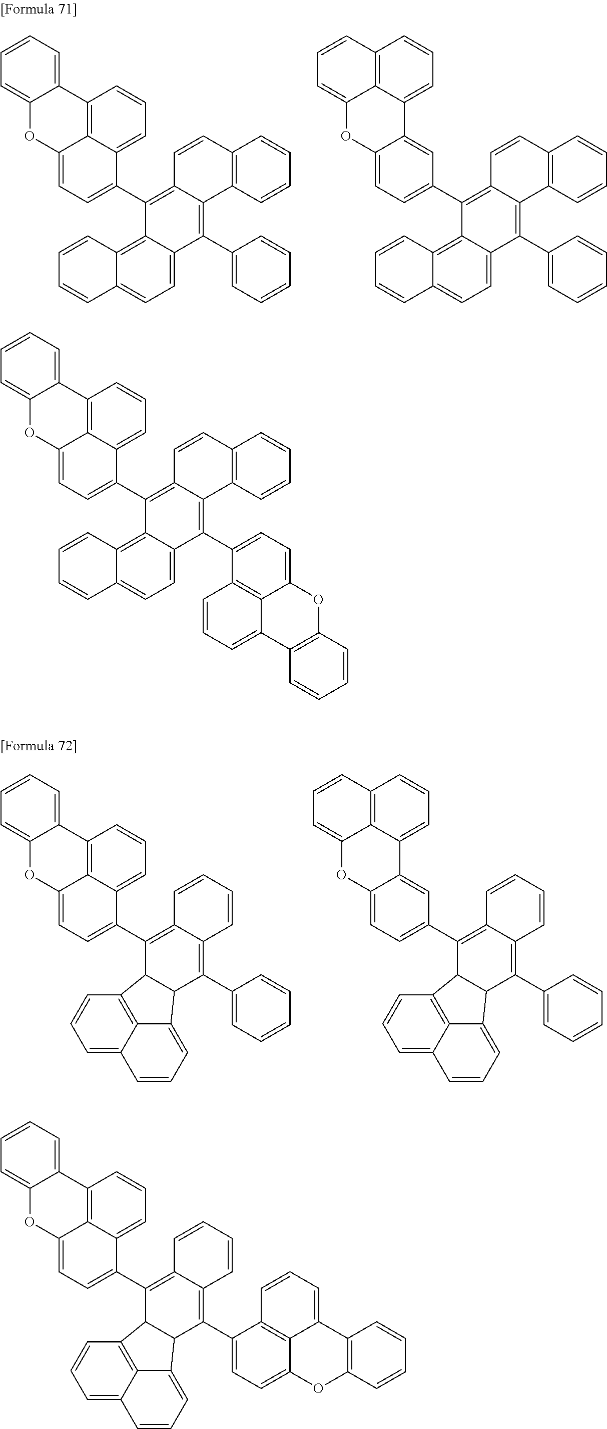

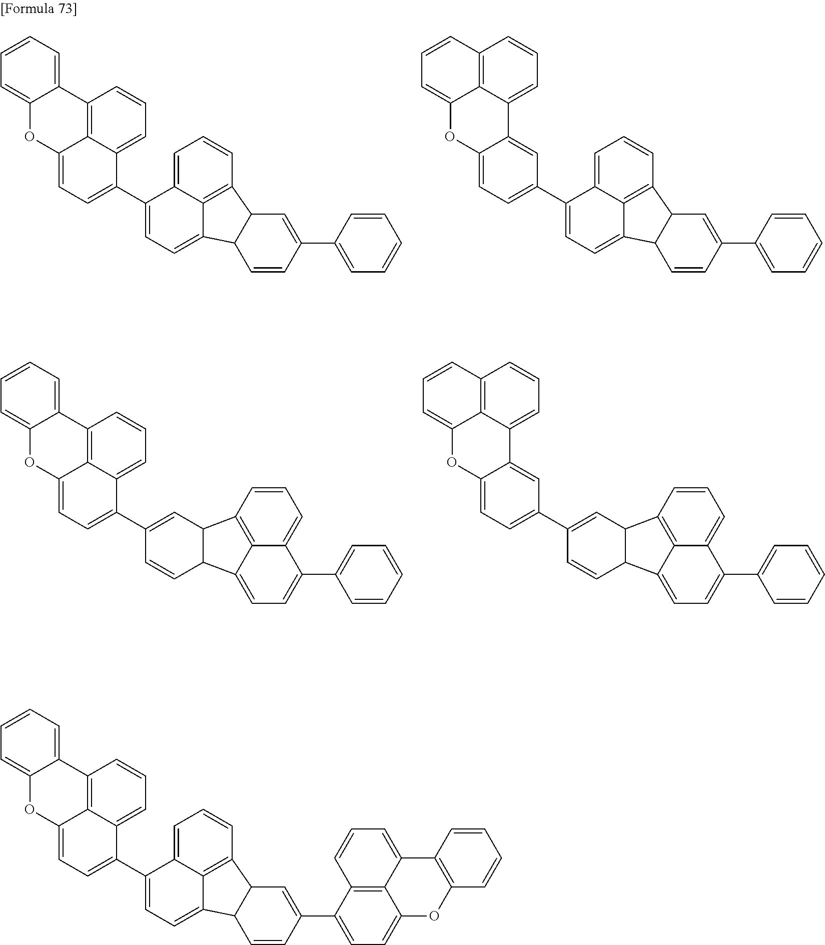 [SMILES: C1=CC2c3ccc(-c4ccc5c6c(cccc46)-c4ccccc4O5)c4cccc(c34)C2C=C1c1ccccc1 ; C1=CC2c3ccc(-c4ccc5c(c4)-c4cccc6cccc(c46)O5)c4cccc(c34)C2C=C1c1ccccc1 ; C1=CC2c3ccc(-c4ccccc4)c4cccc(c34)C2C=C1c1ccc2c3c(cccc13)-c1ccccc1O2 ; C1=CC2c3ccc(-c4ccccc4)c4cccc(c34)C2C=C1c1ccc2c(c1)-c1cccc3cccc(c13)O2 ; C1=CC2c3ccc(-c4ccc5c6c(cccc46)-c4ccccc4O5)c4cccc(c34)C2C=C1c1ccc2c3c(cccc13)-c1ccccc1O2]

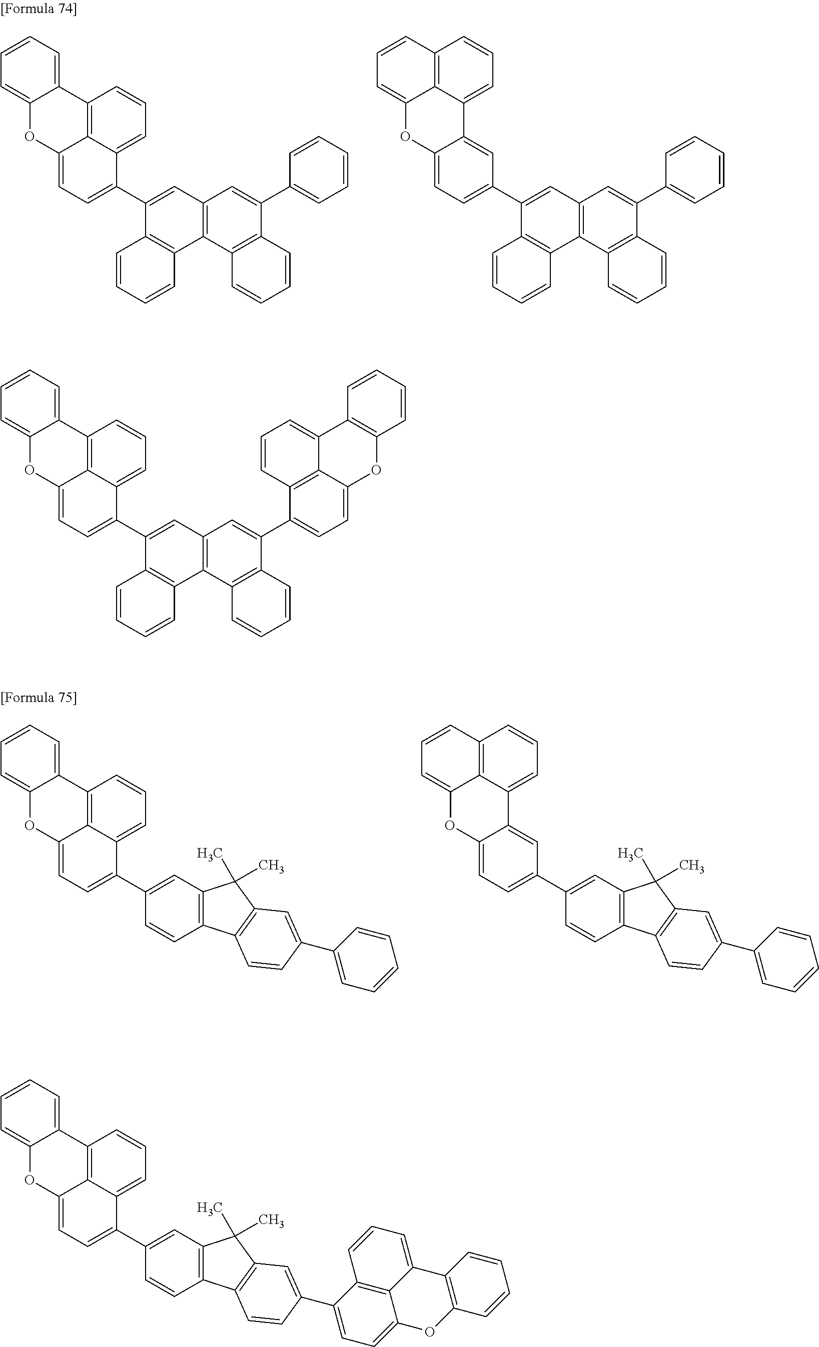 [SMILES: c1ccc(-c2cc3cc(-c4ccc5c6c(cccc46)-c4ccccc4O5)c4ccccc4c3c3ccccc23)cc1 ; c1ccc(-c2cc3cc(-c4ccc5c(c4)-c4cccc6cccc(c46)O5)c4ccccc4c3c3ccccc23)cc1 ; c1ccc2c(c1)Oc1ccc(-c3cc4cc(-c5ccc6c7c(cccc57)-c5ccccc5O6)c5ccccc5c4c4ccccc34)c3cccc-2c13 ; CC1(C)c2cc(-c3ccccc3)ccc2-c2ccc(-c3ccc4c5c(cccc35)-c3ccccc3O4)cc21 ; CC1(C)c2cc(-c3ccccc3)ccc2-c2ccc(-c3ccc4c(c3)-c3cccc5cccc(c35)O4)cc21 ; CC1(C)c2cc(-c3ccc4c5c(cccc35)-c3ccccc3O4)ccc2-c2ccc(-c3ccc4c5c(cccc35)-c3ccccc3O4)cc21]

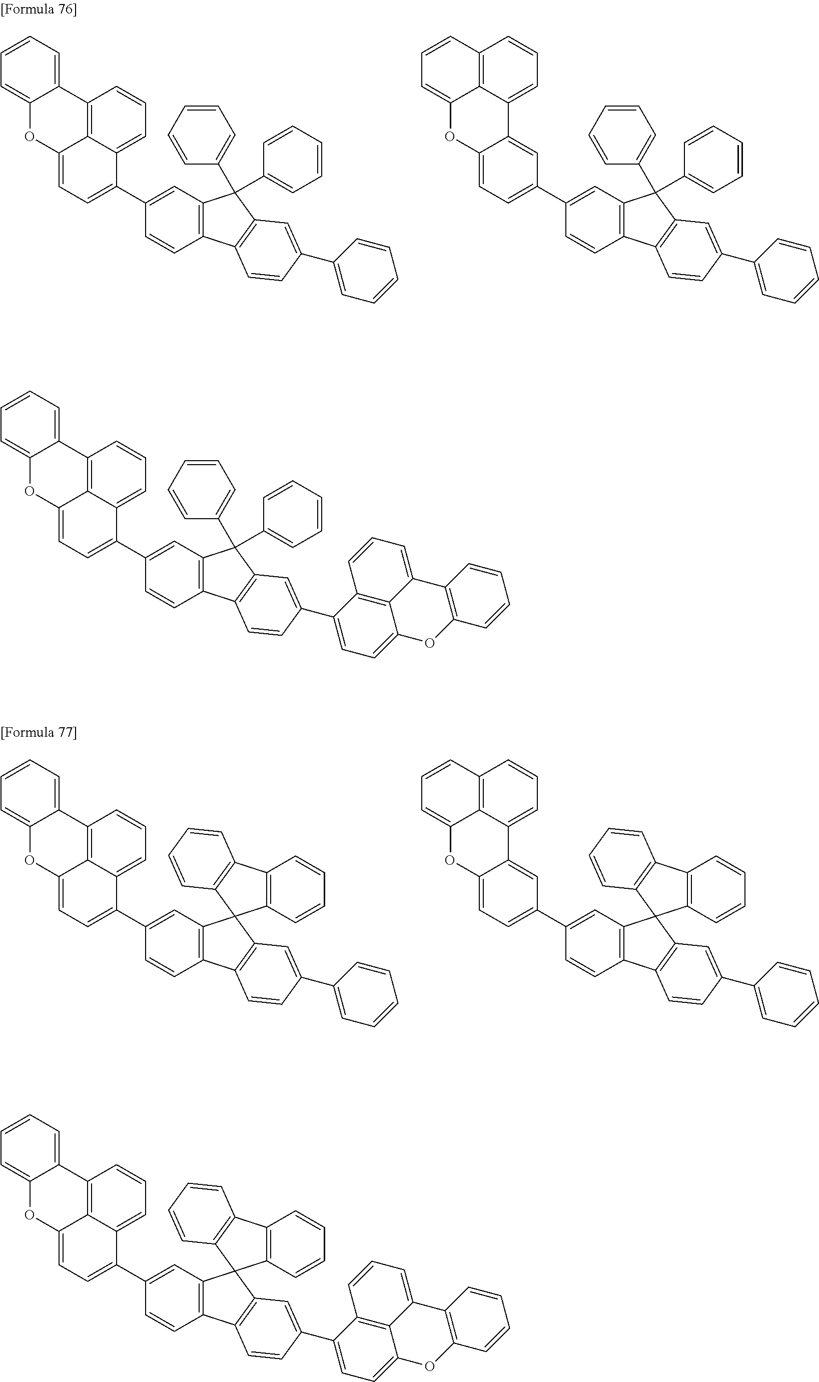

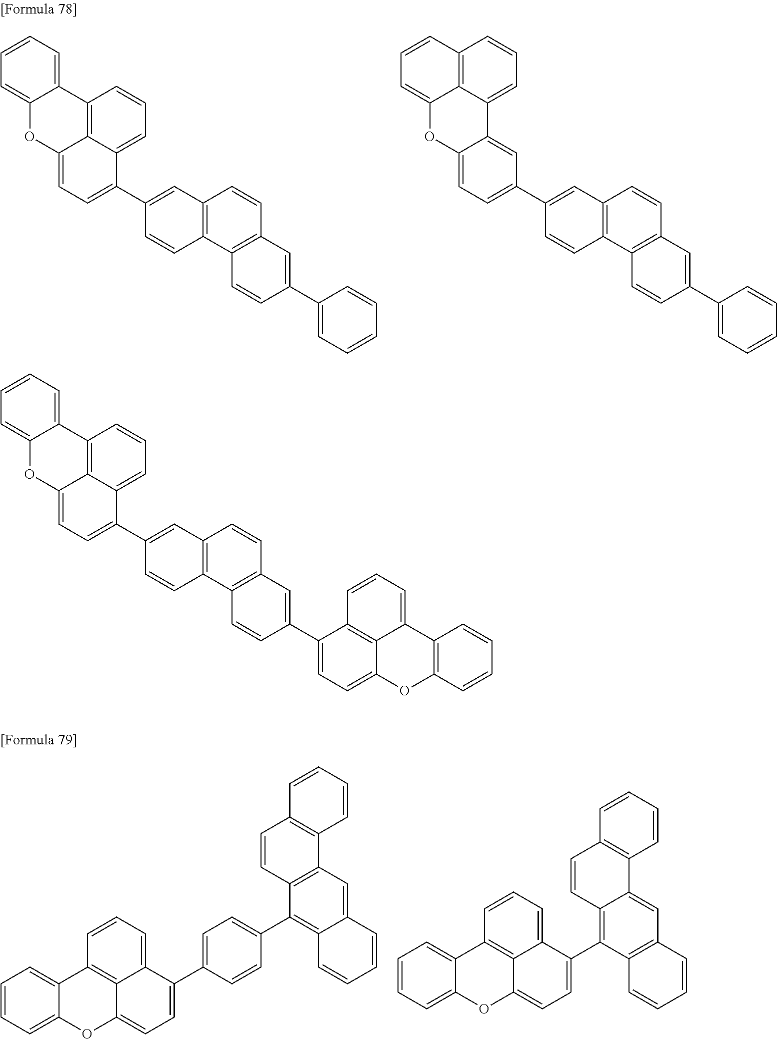

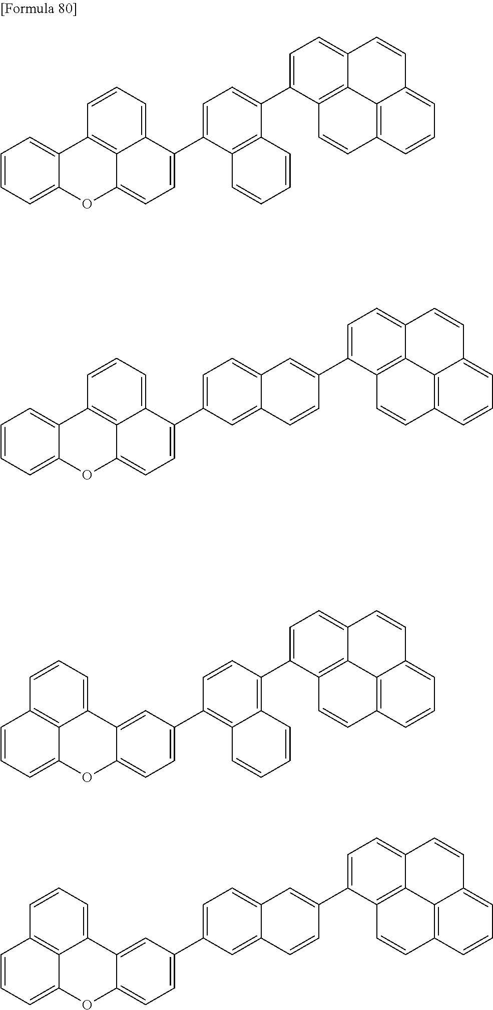 [SMILES: c1ccc2c(c1)Oc1ccc(-c3ccc(-c4ccc5ccc6cccc7ccc4c5c67)c4ccccc34)c3cccc-2c13 ; c1ccc2c(c1)Oc1ccc(-c3ccc4cc(-c5ccc6ccc7cccc8ccc5c6c78)ccc4c3)c3cccc-2c13 ; c1cc2c3c(cccc3c1)-c1cc(-c3ccc(-c4ccc5ccc6cccc7ccc4c5c67)c4ccccc34)ccc1O2 ; c1cc2c3c(cccc3c1)-c1cc(-c3ccc4cc(-c5ccc6ccc7cccc8ccc5c6c78)ccc4c3)ccc1O2]

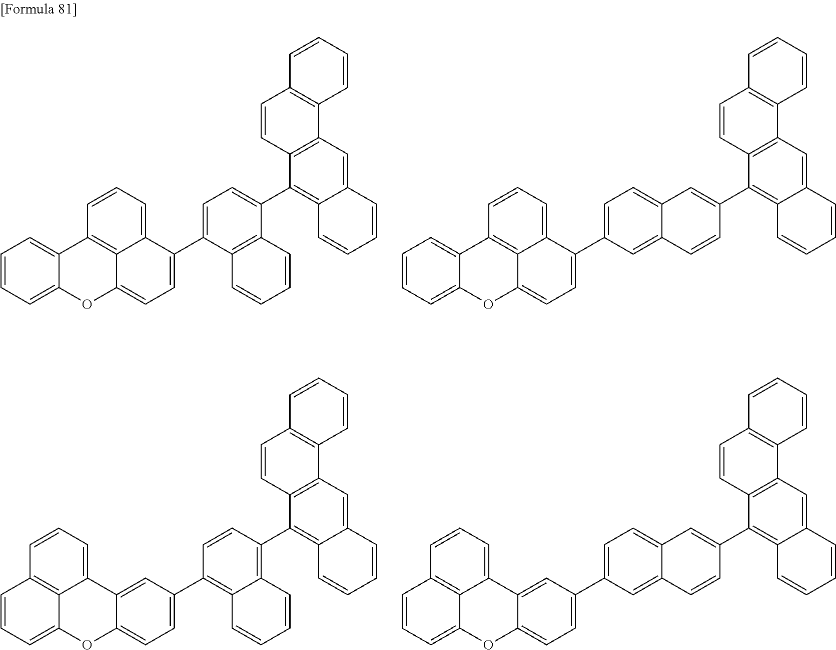

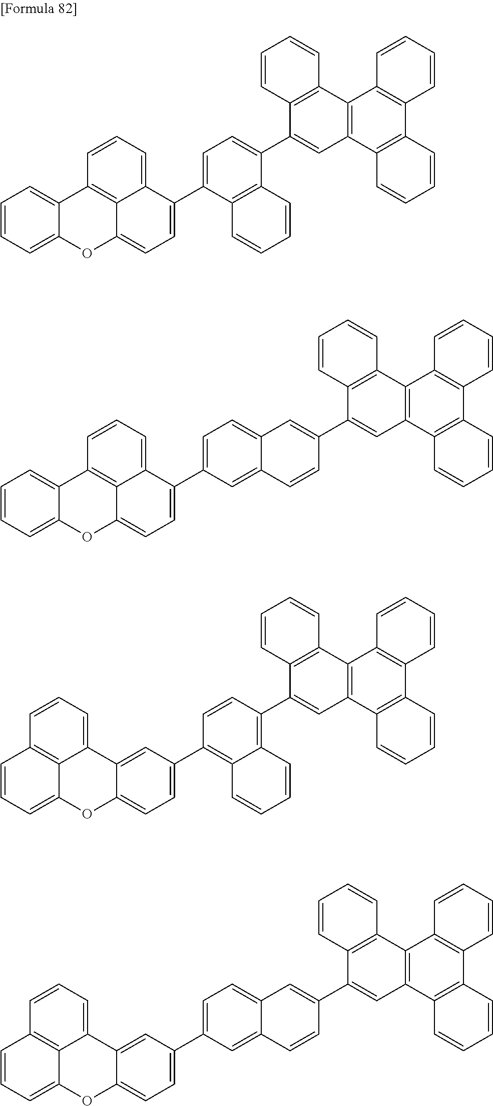 [SMILES: c1ccc2c(c1)Oc1ccc(-c3ccc(-c4cc5c6ccccc6c6ccccc6c5c5ccccc45)c4ccccc34)c3cccc-2c13 ; c1ccc2c(c1)Oc1ccc(-c3ccc4cc(-c5cc6c7ccccc7c7ccccc7c6c6ccccc56)ccc4c3)c3cccc-2c13 ; c1cc2c3c(cccc3c1)-c1cc(-c3ccc(-c4cc5c6ccccc6c6ccccc6c5c5ccccc45)c4ccccc34)ccc1O2 ; c1cc2c3c(cccc3c1)-c1cc(-c3ccc4cc(-c5cc6c7ccccc7c7ccccc7c6c6ccccc56)ccc4c3)ccc1O2]

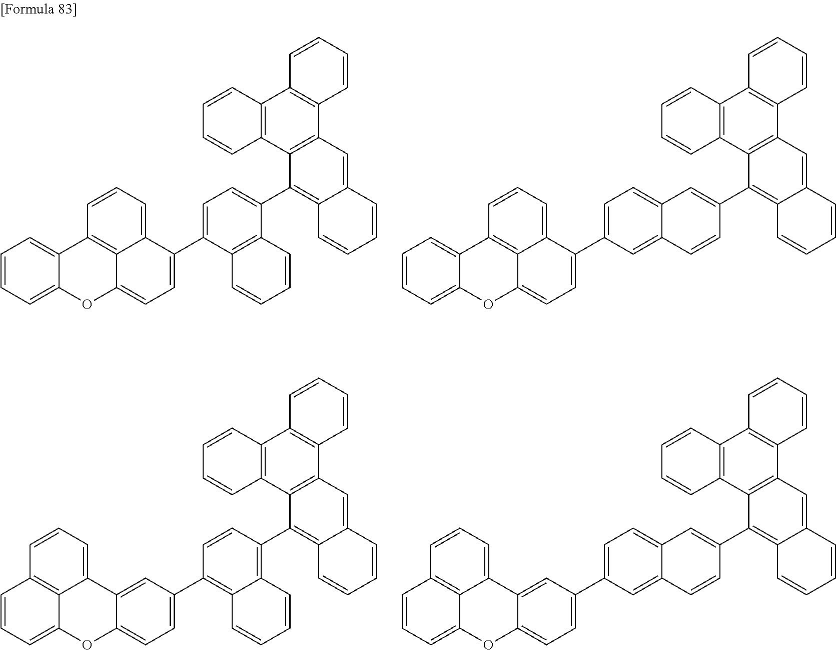

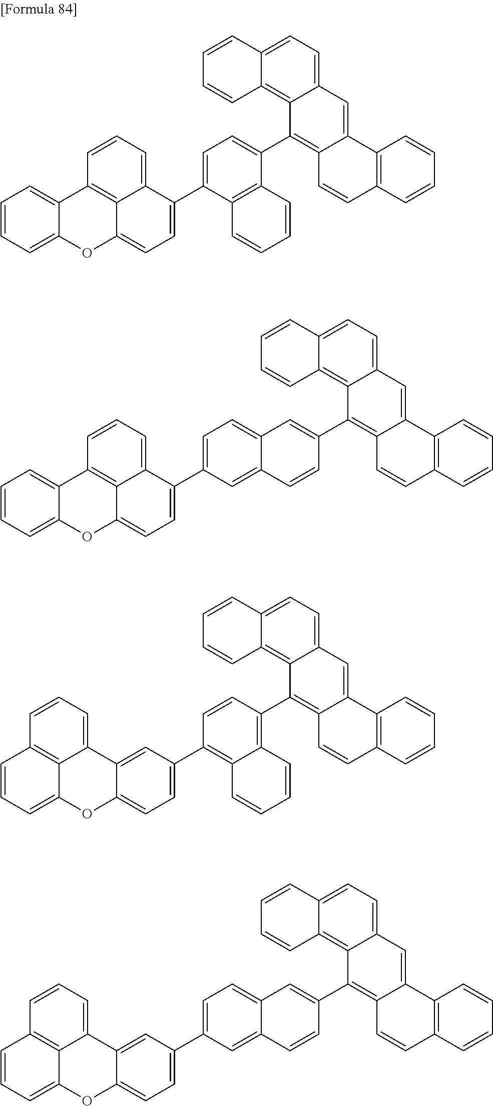 [SMILES: c1ccc2c(c1)Oc1ccc(-c3ccc(-c4c5ccc6ccccc6c5cc5ccc6ccccc6c45)c4ccccc34)c3cccc-2c13 ; c1ccc2c(c1)Oc1ccc(-c3ccc4cc(-c5c6ccc7ccccc7c6cc6ccc7ccccc7c56)ccc4c3)c3cccc-2c13 ; c1cc2c3c(cccc3c1)-c1cc(-c3ccc(-c4c5ccc6ccccc6c5cc5ccc6ccccc6c45)c4ccccc34)ccc1O2 ; c1cc2c3c(cccc3c1)-c1cc(-c3ccc4cc(-c5c6ccc7ccccc7c6cc6ccc7ccccc7c56)ccc4c3)ccc1O2]

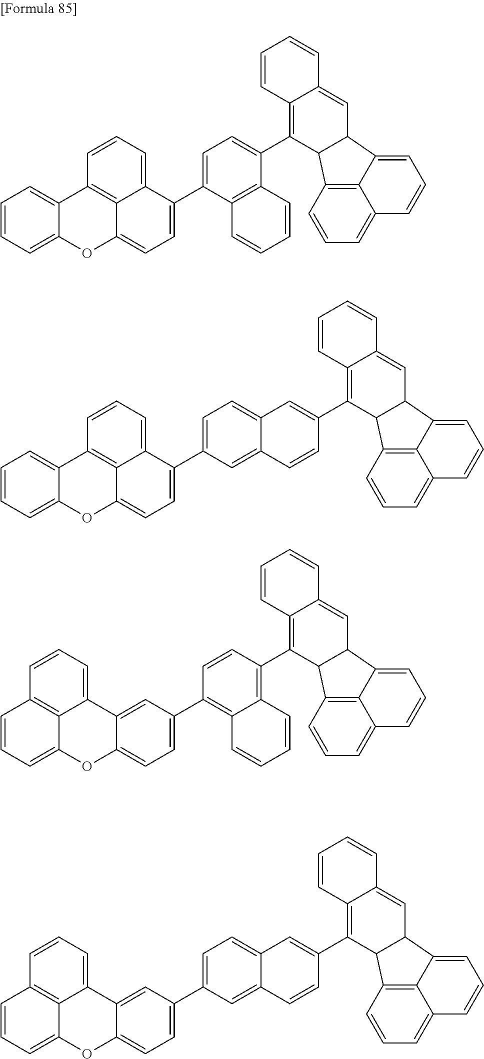 [SMILES: C1=c2ccccc2=C(c2ccc(-c3ccc4c5c(cccc35)-c3ccccc3O4)c3ccccc23)C2c3cccc4cccc(c34)C12 ; C1=c2ccccc2=C(c2ccc3cc(-c4ccc5c6c(cccc46)-c4ccccc4O5)ccc3c2)C2c3cccc4cccc(c34)C12 ; C1=c2ccccc2=C(c2ccc(-c3ccc4c(c3)-c3cccc5cccc(c35)O4)c3ccccc23)C2c3cccc4cccc(c34)C12 ; C1=c2ccccc2=C(c2ccc3cc(-c4ccc5c(c4)-c4cccc6cccc(c46)O5)ccc3c2)C2c3cccc4cccc(c34)C12]

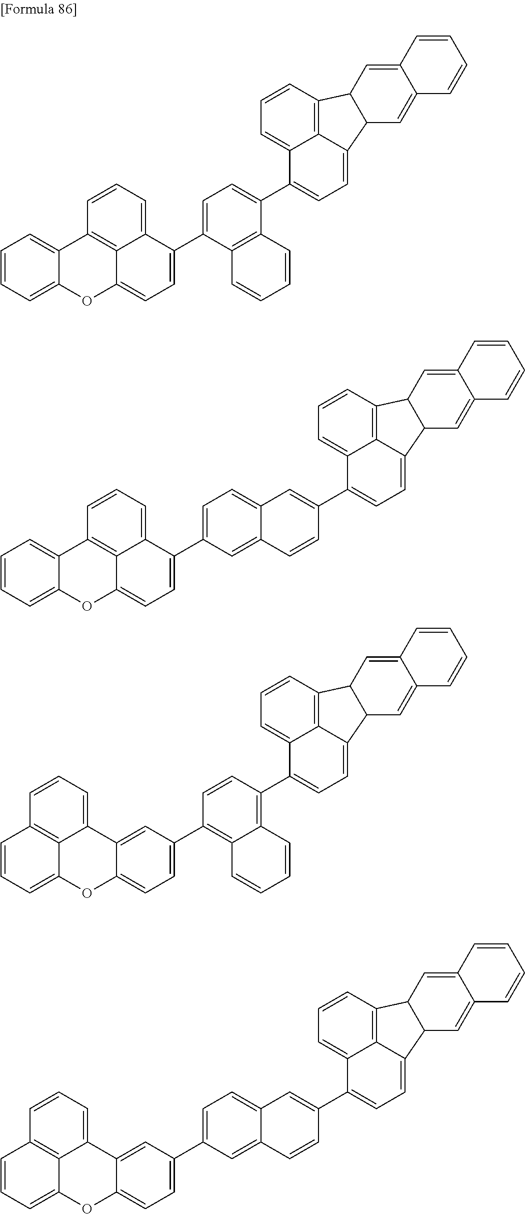 [SMILES: C1=c2ccccc2=CC2c3ccc(-c4ccc(-c5ccc6c7c(cccc57)-c5ccccc5O6)c5ccccc45)c4cccc(c34)C12 ; C1=c2ccccc2=CC2c3ccc(-c4ccc5cc(-c6ccc7c8c(cccc68)-c6ccccc6O7)ccc5c4)c4cccc(c34)C12 ; C1=c2ccccc2=CC2c3ccc(-c4ccc(-c5ccc6c(c5)-c5cccc7cccc(c57)O6)c5ccccc45)c4cccc(c34)C12 ; C1=c2ccccc2=CC2c3ccc(-c4ccc5cc(-c6ccc7c(c6)-c6cccc8cccc(c68)O7)ccc5c4)c4cccc(c34)C12]

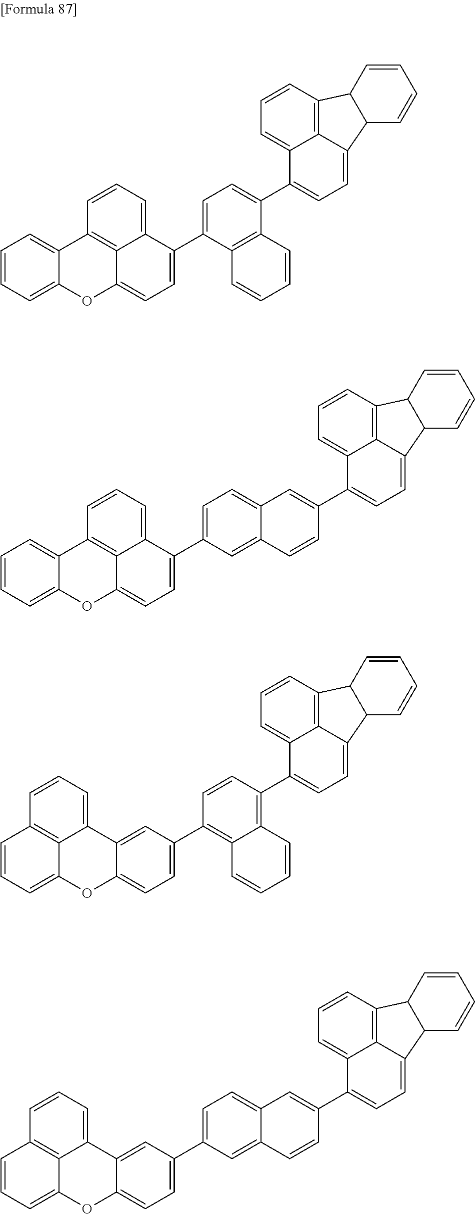 [SMILES: C1=CC2c3cccc4c(-c5ccc(-c6ccc7c8c(cccc68)-c6ccccc6O7)c6ccccc56)ccc(c34)C2C=C1 ; C1=CC2c3cccc4c(-c5ccc6cc(-c7ccc8c9c(cccc79)-c7ccccc7O8)ccc6c5)ccc(c34)C2C=C1 ; C1=CC2c3cccc4c(-c5ccc(-c6ccc7c(c6)-c6cccc8cccc(c68)O7)c6ccccc56)ccc(c34)C2C=C1 ; C1=CC2c3cccc4c(-c5ccc6cc(-c7ccc8c(c7)-c7cccc9cccc(c79)O8)ccc6c5)ccc(c34)C2C=C1]

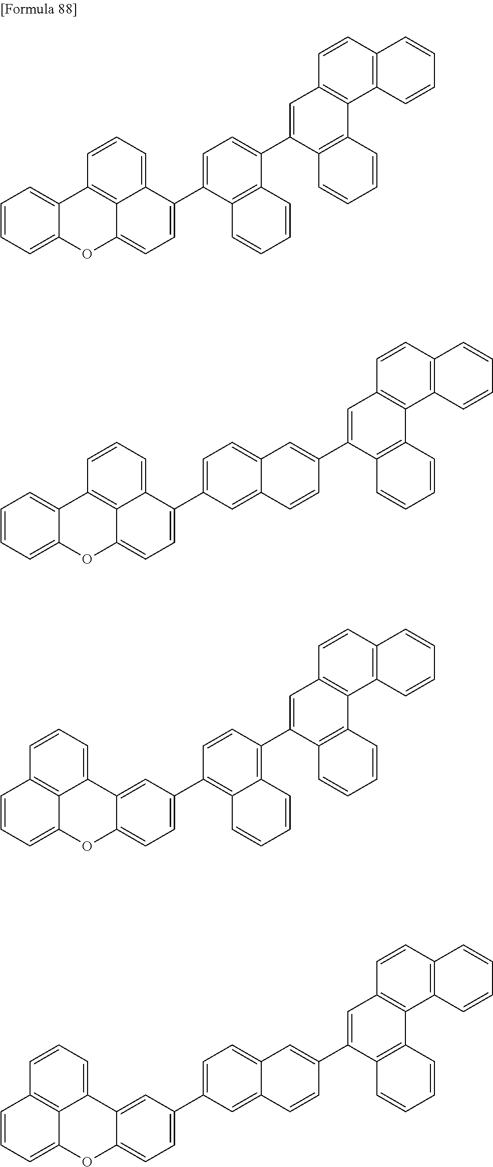 [SMILES: c1ccc2c(c1)Oc1ccc(-c3ccc(-c4cc5ccc6ccccc6c5c5ccccc45)c4ccccc34)c3cccc-2c13 ; c1ccc2c(c1)Oc1ccc(-c3ccc4cc(-c5cc6ccc7ccccc7c6c6ccccc56)ccc4c3)c3cccc-2c13 ; c1cc2c3c(cccc3c1)-c1cc(-c3ccc(-c4cc5ccc6ccccc6c5c5ccccc45)c4ccccc34)ccc1O2 ; c1cc2c3c(cccc3c1)-c1cc(-c3ccc4cc(-c5cc6ccc7ccccc7c6c6ccccc56)ccc4c3)ccc1O2]

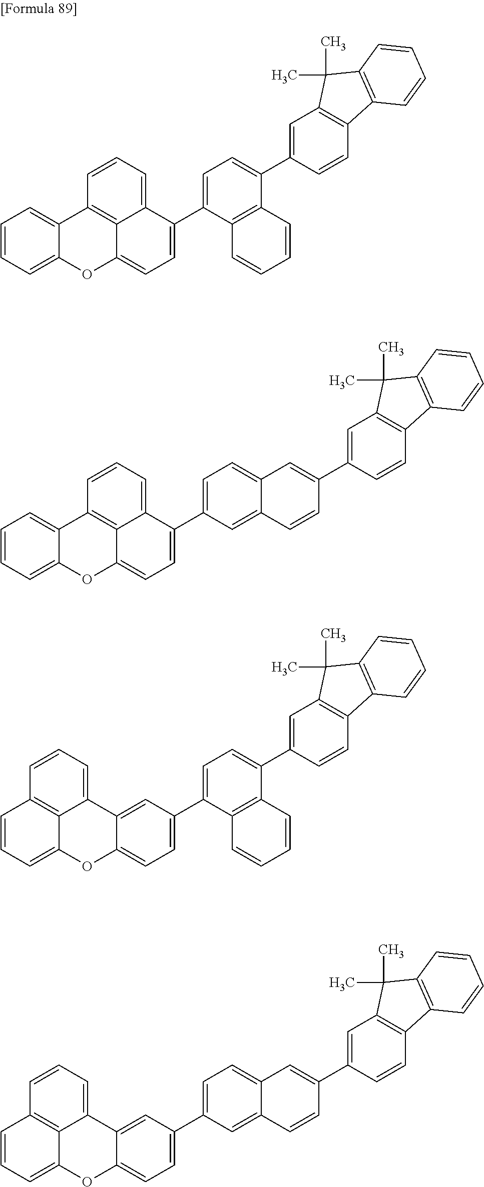 [SMILES: CC1(C)c2ccccc2-c2ccc(-c3ccc(-c4ccc5c6c(cccc46)-c4ccccc4O5)c4ccccc34)cc21 ; CC1(C)c2ccccc2-c2ccc(-c3ccc4cc(-c5ccc6c7c(cccc57)-c5ccccc5O6)ccc4c3)cc21 ; CC1(C)c2ccccc2-c2ccc(-c3ccc(-c4ccc5c(c4)-c4cccc6cccc(c46)O5)c4ccccc34)cc21 ; CC1(C)c2ccccc2-c2ccc(-c3ccc4cc(-c5ccc6c(c5)-c5cccc7cccc(c57)O6)ccc4c3)cc21]

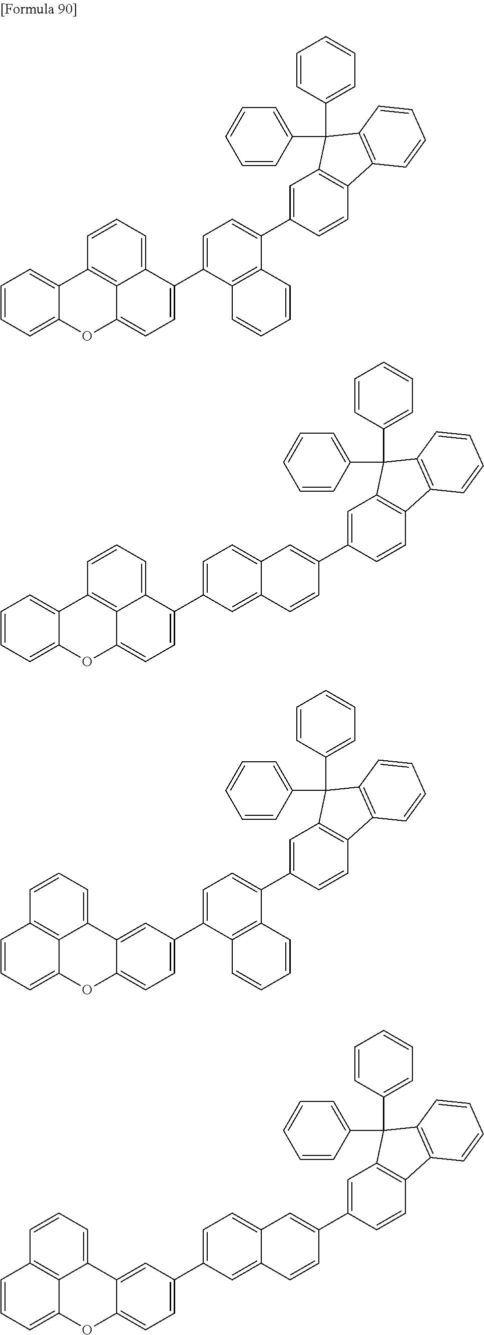 [SMILES: c1ccc(C2(c3ccccc3)c3ccccc3-c3ccc(-c4ccc(-c5ccc6c7c(cccc57)-c5ccccc5O6)c5ccccc45)cc32)cc1 ; c1ccc(C2(c3ccccc3)c3ccccc3-c3ccc(-c4ccc5cc(-c6ccc7c8c(cccc68)-c6ccccc6O7)ccc5c4)cc32)cc1 ; c1ccc(C2(c3ccccc3)c3ccccc3-c3ccc(-c4ccc(-c5ccc6c(c5)-c5cccc7cccc(c57)O6)c5ccccc45)cc32)cc1 ; c1ccc(C2(c3ccccc3)c3ccccc3-c3ccc(-c4ccc5cc(-c6ccc7c(c6)-c6cccc8cccc(c68)O7)ccc5c4)cc32)cc1]

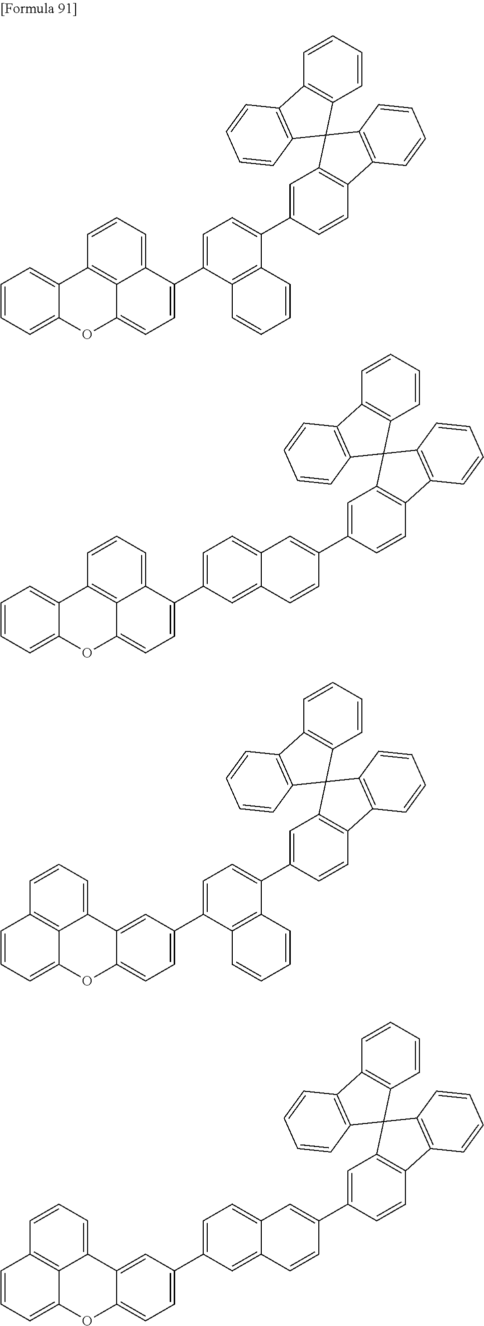 [SMILES: c1ccc2c(c1)Oc1ccc(-c3ccc(-c4ccc5c(c4)C4(c6ccccc6-c6ccccc64)c4ccccc4-5)c4ccccc34)c3cccc-2c13 ; c1ccc2c(c1)Oc1ccc(-c3ccc4cc(-c5ccc6c(c5)C5(c7ccccc7-c7ccccc75)c5ccccc5-6)ccc4c3)c3cccc-2c13 ; c1ccc2c(c1)-c1ccccc1C21c2ccccc2-c2ccc(-c3ccc(-c4ccc5c(c4)-c4cccc6cccc(c46)O5)c4ccccc34)cc21 ; c1ccc2c(c1)-c1ccccc1C21c2ccccc2-c2ccc(-c3ccc4cc(-c5ccc6c(c5)-c5cccc7cccc(c57)O6)ccc4c3)cc21]

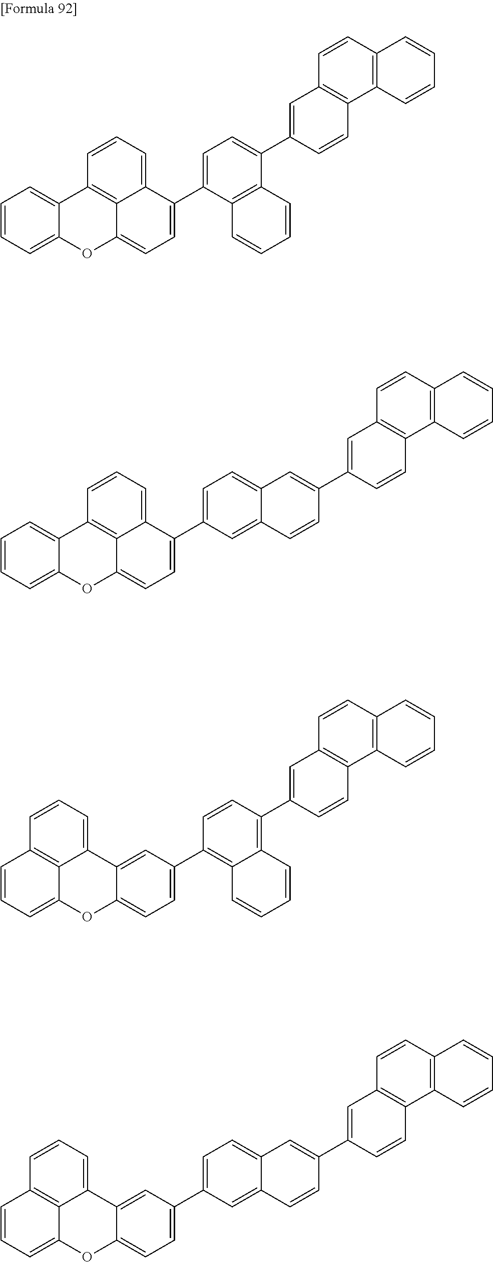 [SMILES: c1ccc2c(c1)Oc1ccc(-c3ccc(-c4ccc5c(ccc6ccccc65)c4)c4ccccc34)c3cccc-2c13 ; c1ccc2c(c1)Oc1ccc(-c3ccc4cc(-c5ccc6c(ccc7ccccc76)c5)ccc4c3)c3cccc-2c13 ; c1cc2c3c(cccc3c1)-c1cc(-c3ccc(-c4ccc5c(ccc6ccccc65)c4)c4ccccc34)ccc1O2 ; c1cc2c3c(cccc3c1)-c1cc(-c3ccc4cc(-c5ccc6c(ccc7ccccc76)c5)ccc4c3)ccc1O2]

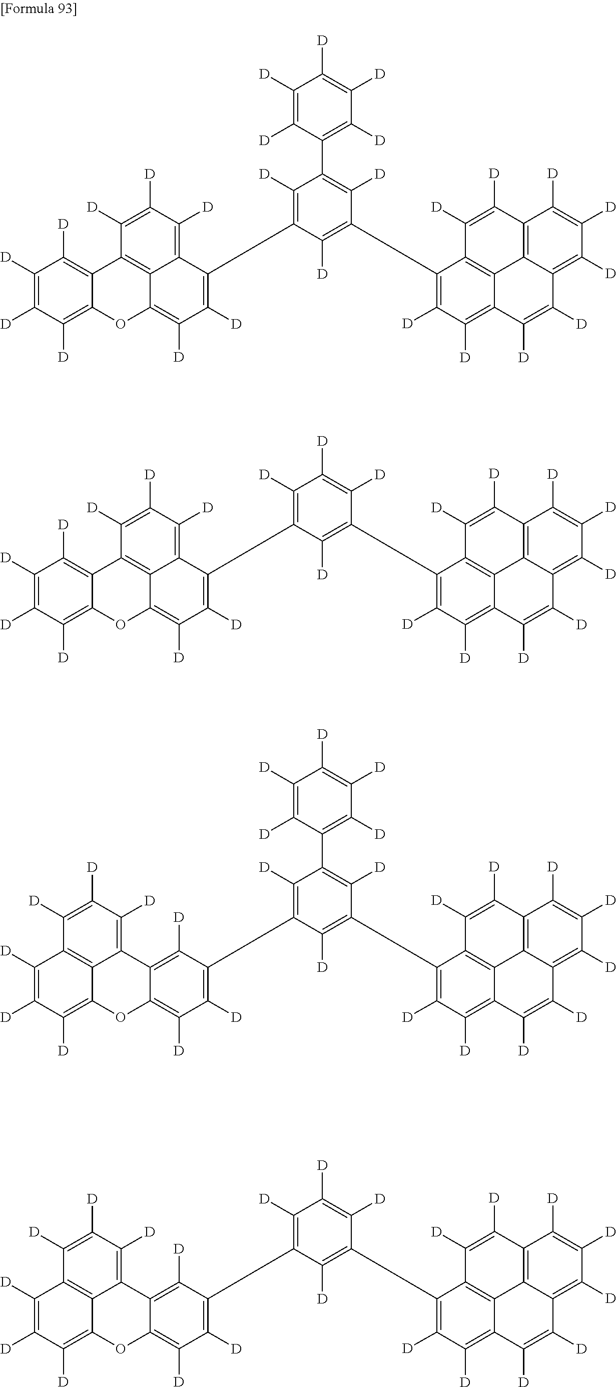

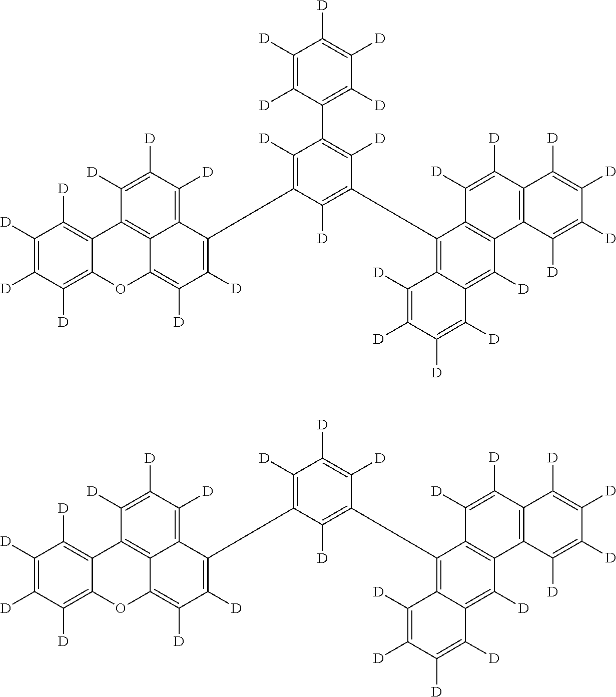 [SMILES: [2H]c1c([2H])c([2H])c(-c2c([2H])c(-c3c([2H])c([2H])c4c5c(c([2H])c([2H])c([2H])c35)-c3c([2H])c([2H])c([2H])c([2H])c3O4)c([2H])c(-c3c4c([2H])c([2H])c([2H])c([2H])c4c([2H])c4c3c([2H])c([2H])c3c([2H])c([2H])c([2H])c([2H])c34)c2[2H])c([2H])c1[2H] ; [2H]c1c([2H])c([2H])c2c(c1[2H])Oc1c([2H])c([2H])c(-c3c([2H])c([2H])c([2H])c(-c4c5c([2H])c([2H])c([2H])c([2H])c5c([2H])c5c4c([2H])c([2H])c4c([2H])c([2H])c([2H])c([2H])c45)c3[2H])c3c([2H])c([2H])c([2H])c-2c13]

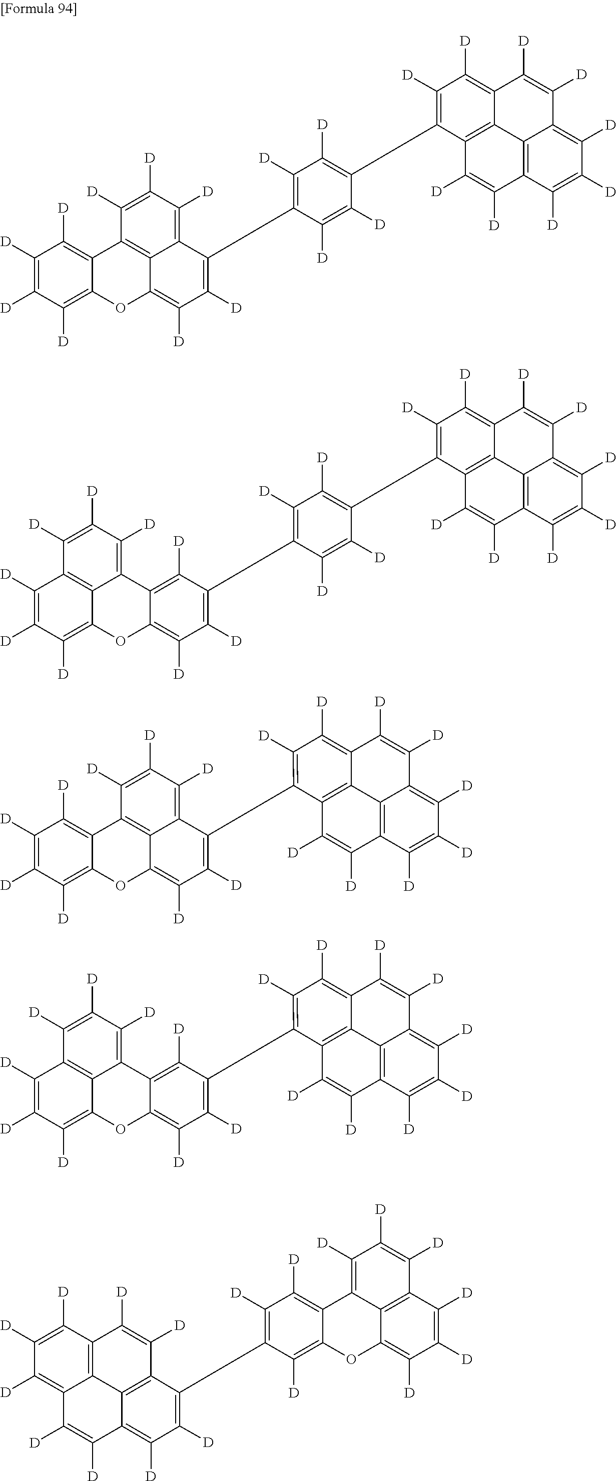

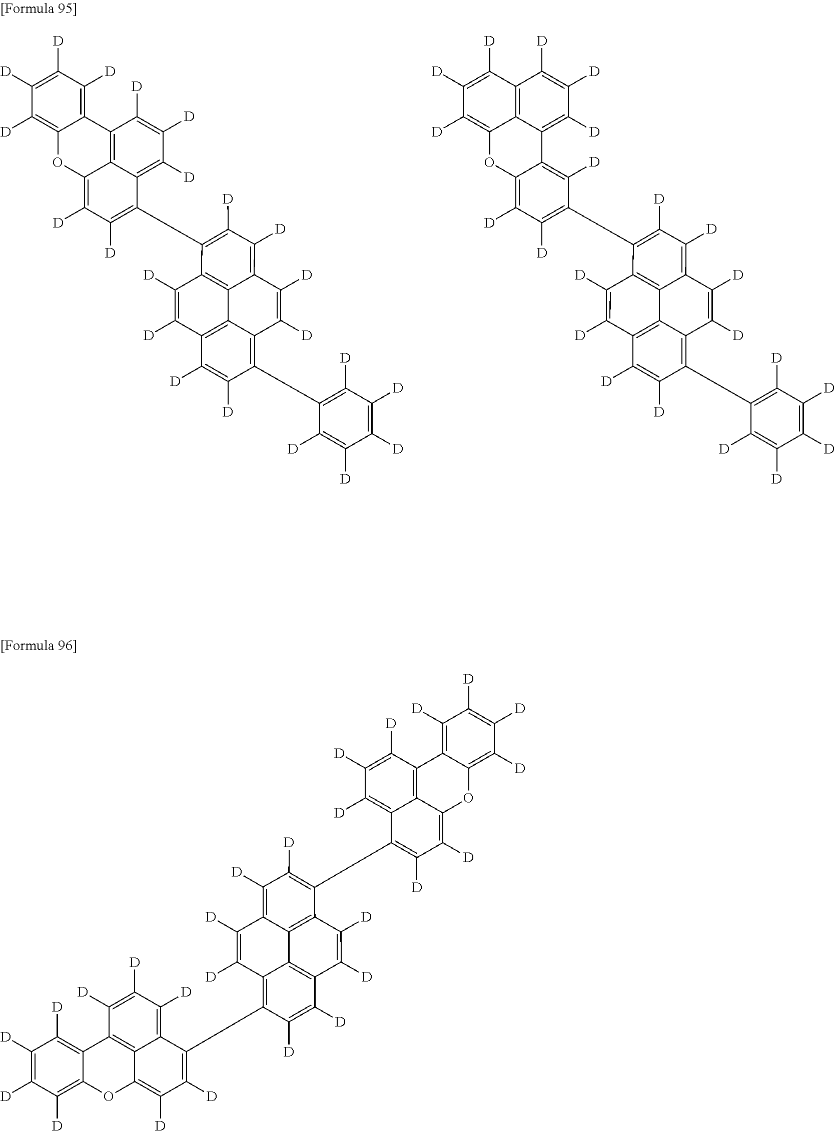

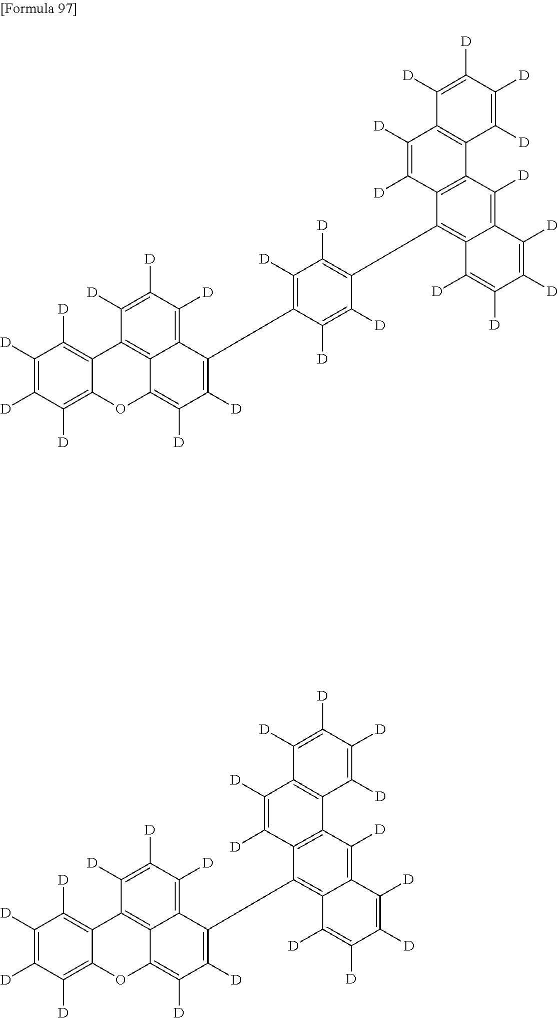 [SMILES: [2H]c1c([2H])c([2H])c2c(c1[2H])Oc1c([2H])c([2H])c(-c3c([2H])c([2H])c(-c4c5c([2H])c([2H])c([2H])c([2H])c5c([2H])c5c4c([2H])c([2H])c4c([2H])c([2H])c([2H])c([2H])c45)c([2H])c3[2H])c3c([2H])c([2H])c([2H])c-2c13 ; [2H]c1c([2H])c([2H])c2c(c1[2H])Oc1c([2H])c([2H])c(-c3c4c([2H])c([2H])c([2H])c([2H])c4c([2H])c4c3c([2H])c([2H])c3c([2H])c([2H])c([2H])c([2H])c34)c3c([2H])c([2H])c([2H])c-2c13]

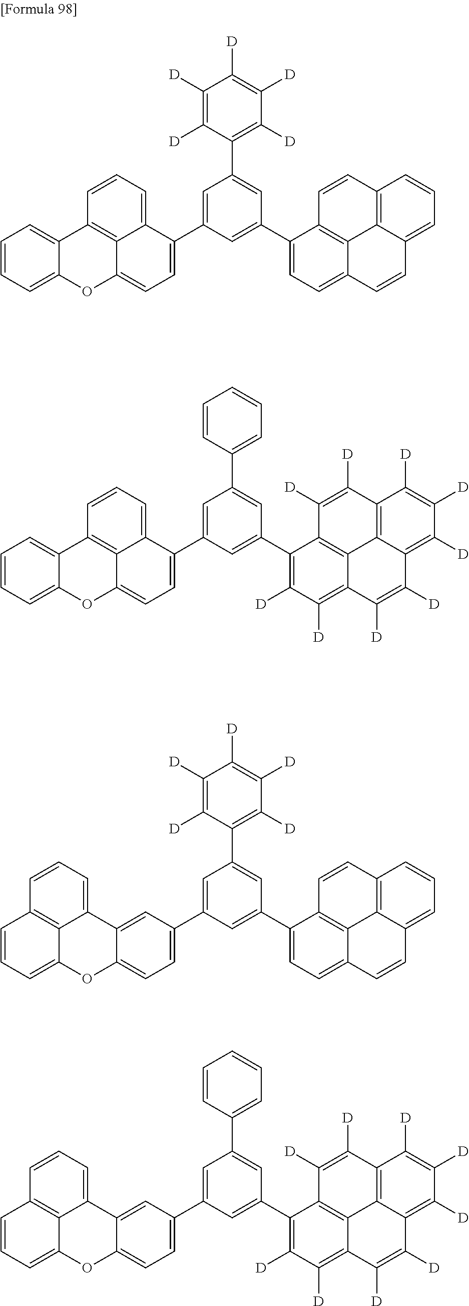 [SMILES: [2H]c1c([2H])c([2H])c(-c2cc(-c3ccc4c5c(cccc35)-c3ccccc3O4)cc(-c3ccc4ccc5cccc6ccc3c4c56)c2)c([2H])c1[2H] ; [2H]c1c([2H])c2c([2H])c([2H])c3c([2H])c([2H])c(-c4cc(-c5ccccc5)cc(-c5ccc6c7c(cccc57)-c5ccccc5O6)c4)c4c([2H])c([2H])c(c1[2H])c2c34 ; [2H]c1c([2H])c([2H])c(-c2cc(-c3ccc4c(c3)-c3cccc5cccc(c35)O4)cc(-c3ccc4ccc5cccc6ccc3c4c56)c2)c([2H])c1[2H] ; [2H]c1c([2H])c2c([2H])c([2H])c3c([2H])c([2H])c(-c4cc(-c5ccccc5)cc(-c5ccc6c(c5)-c5cccc7cccc(c57)O6)c4)c4c([2H])c([2H])c(c1[2H])c2c34]

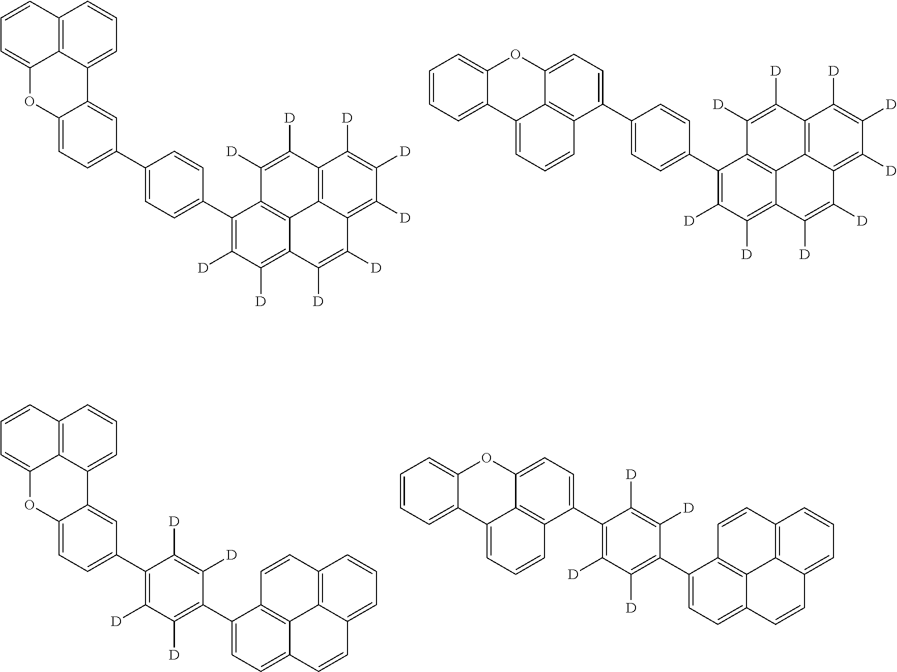 [SMILES: [2H]c1c([2H])c2c([2H])c([2H])c3c([2H])c([2H])c(-c4ccc(-c5ccc6c(c5)-c5cccc7cccc(c57)O6)cc4)c4c([2H])c([2H])c(c1[2H])c2c34 ; [2H]c1c([2H])c2c([2H])c([2H])c3c([2H])c([2H])c(-c4ccc(-c5ccc6c7c(cccc57)-c5ccccc5O6)cc4)c4c([2H])c([2H])c(c1[2H])c2c34 ; [2H]c1c([2H])c(-c2ccc3ccc4cccc5ccc2c3c45)c([2H])c([2H])c1-c1ccc2c(c1)-c1cccc3cccc(c13)O2 ; [2H]c1c([2H])c(-c2ccc3ccc4cccc5ccc2c3c45)c([2H])c([2H])c1-c1ccc2c3c(cccc13)-c1ccccc1O2]

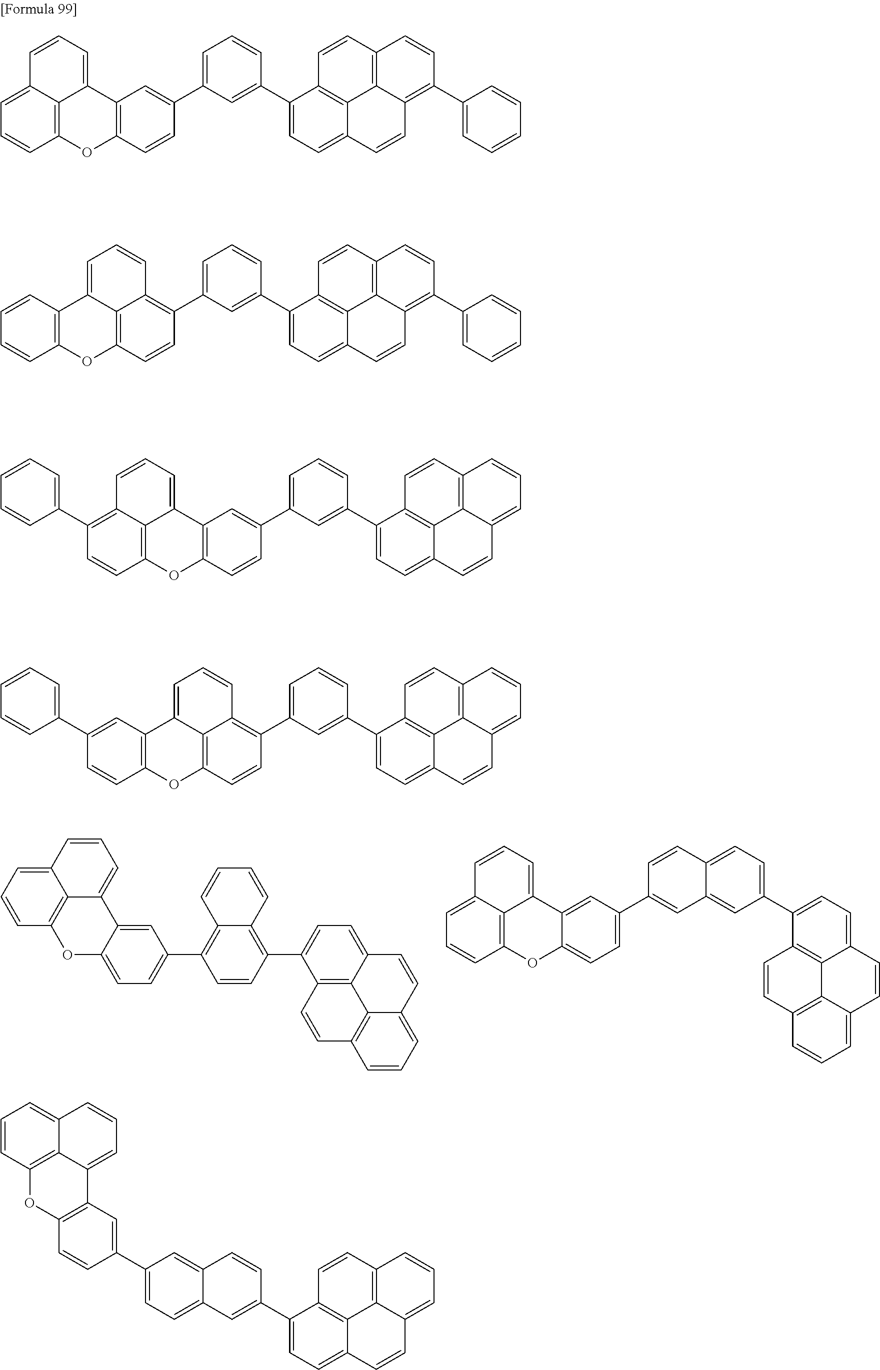

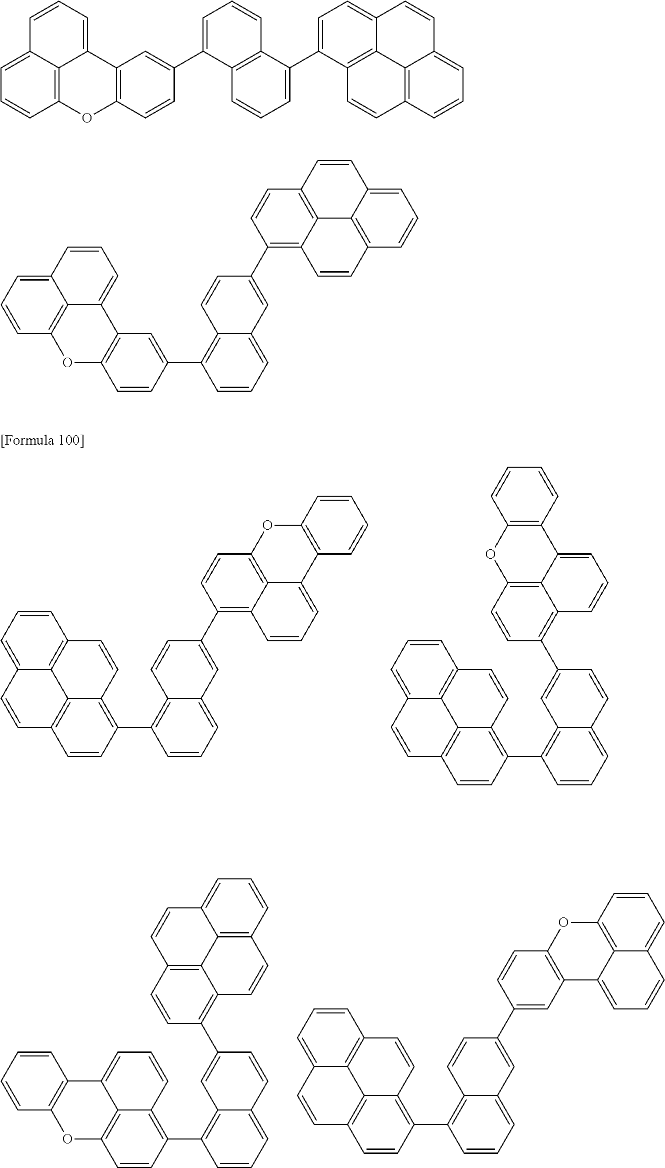

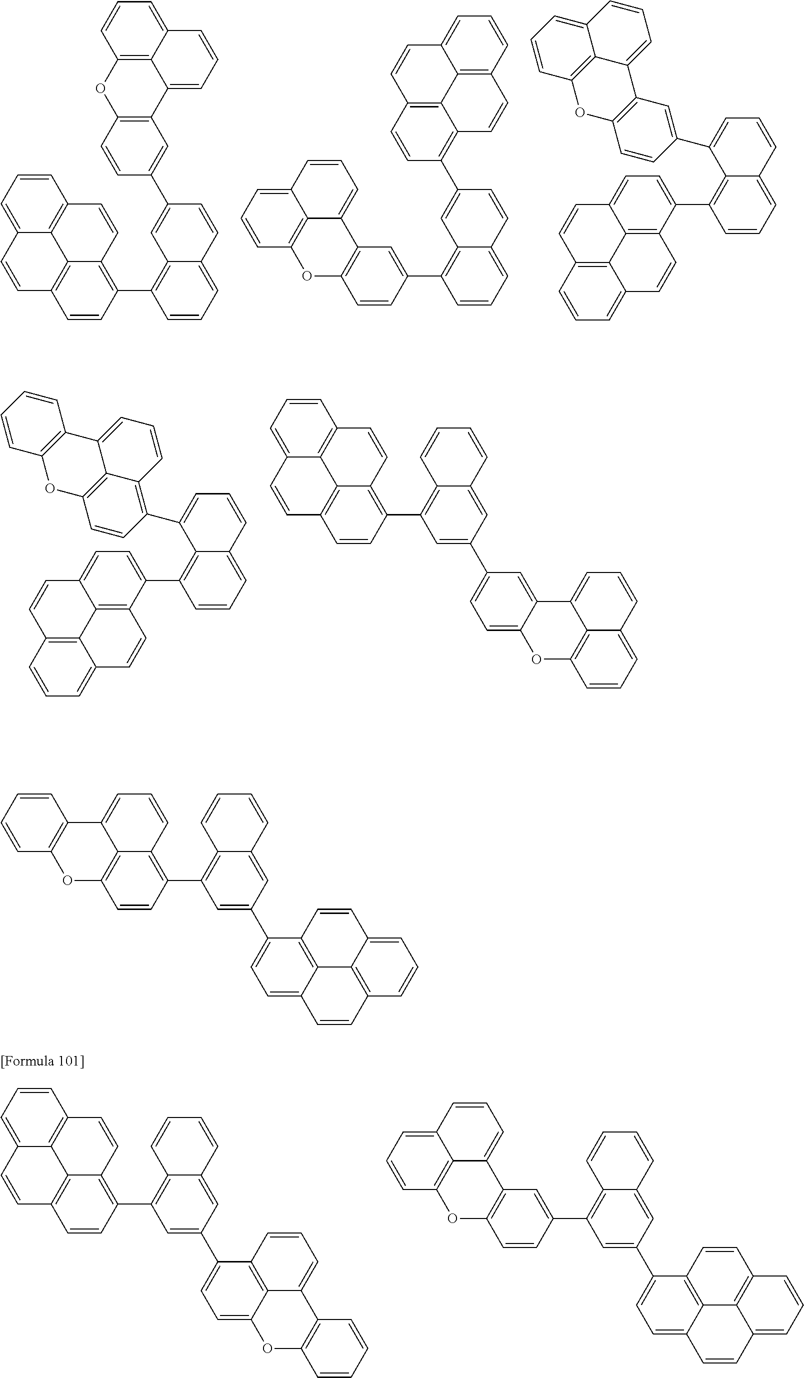

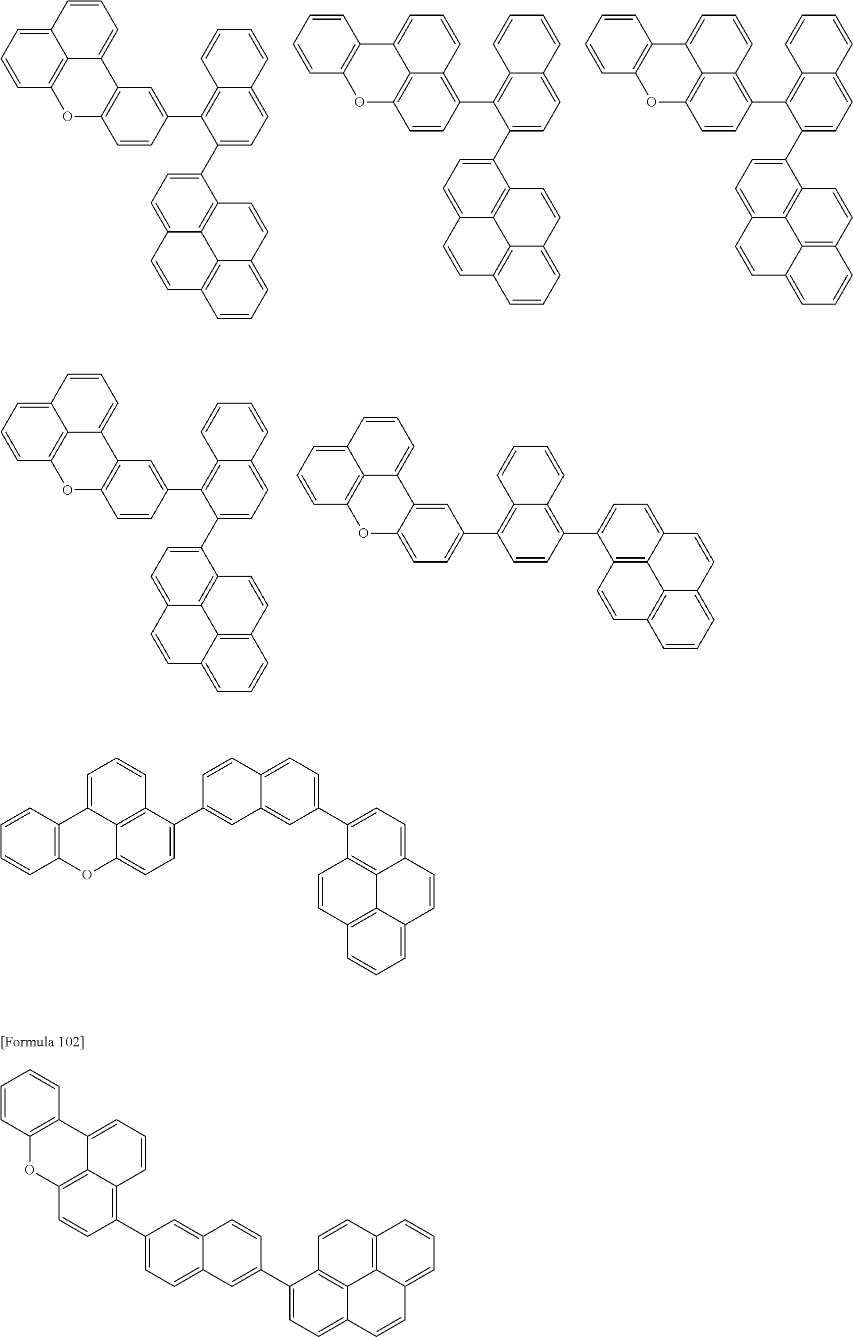

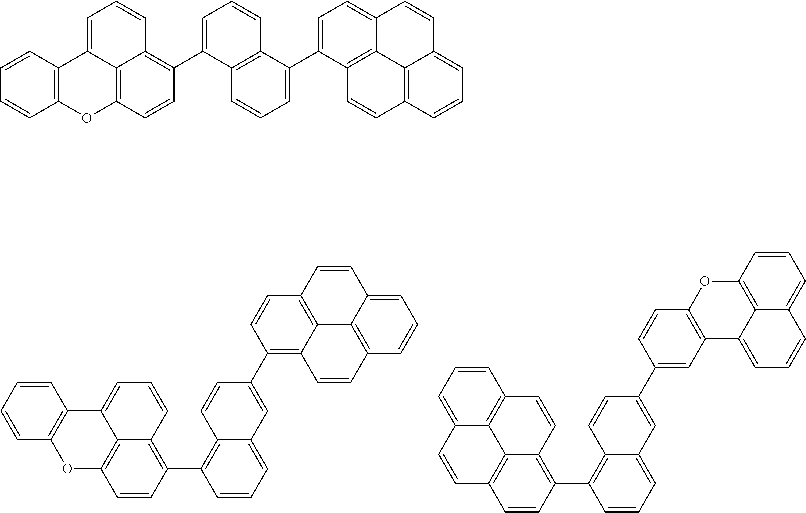 [SMILES: c1ccc2c(c1)Oc1ccc(-c3cccc4c(-c5ccc6ccc7cccc8ccc5c6c78)cccc34)c3cccc-2c13 ; c1ccc2c(c1)Oc1ccc(-c3cccc4cc(-c5ccc6ccc7cccc8ccc5c6c78)ccc34)c3cccc-2c13 ; c1cc(-c2ccc3ccc4cccc5ccc2c3c45)c2ccc(-c3ccc4c(c3)-c3cccc5cccc(c35)O4)cc2c1]

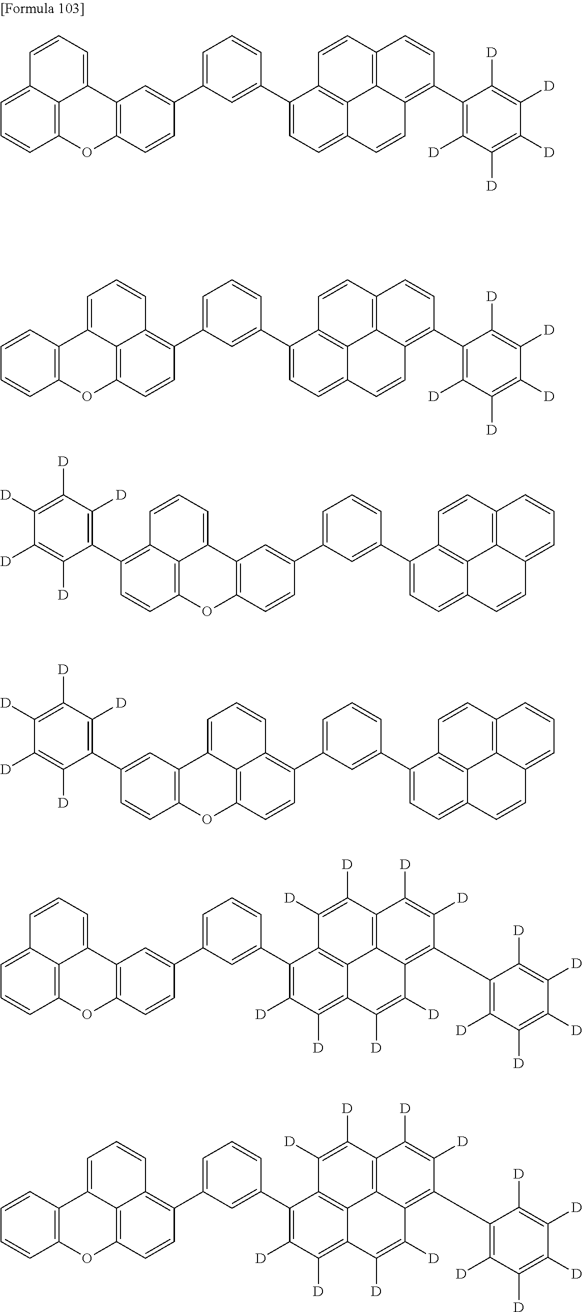

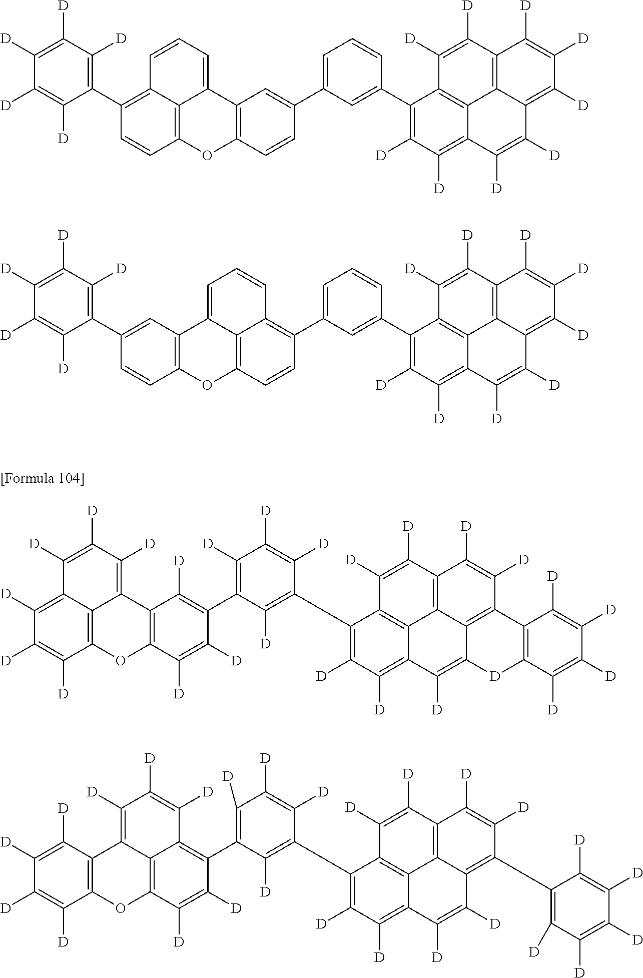

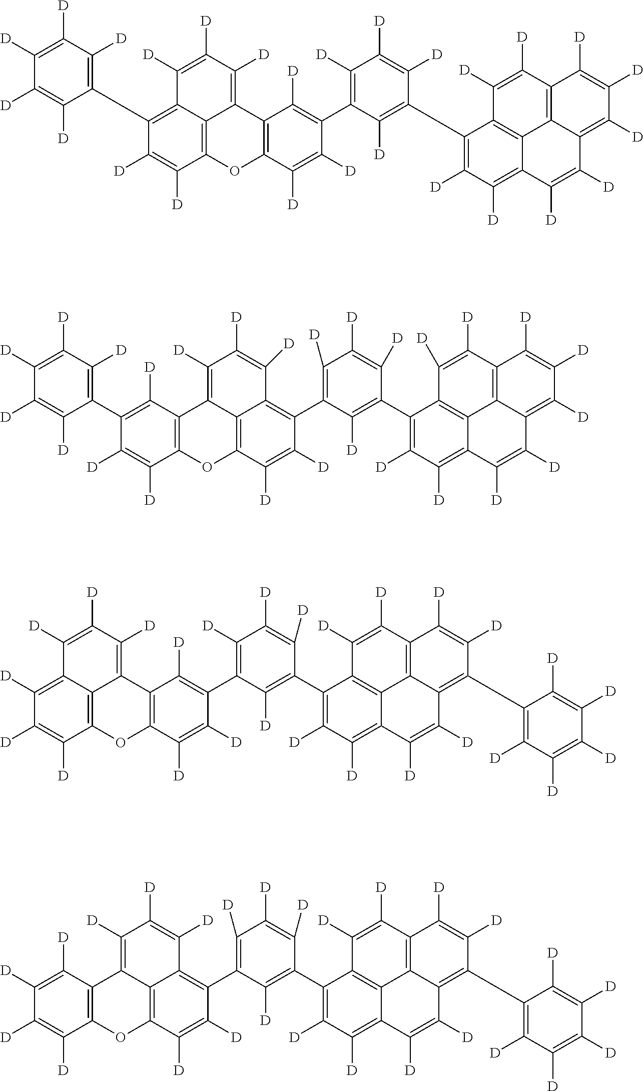

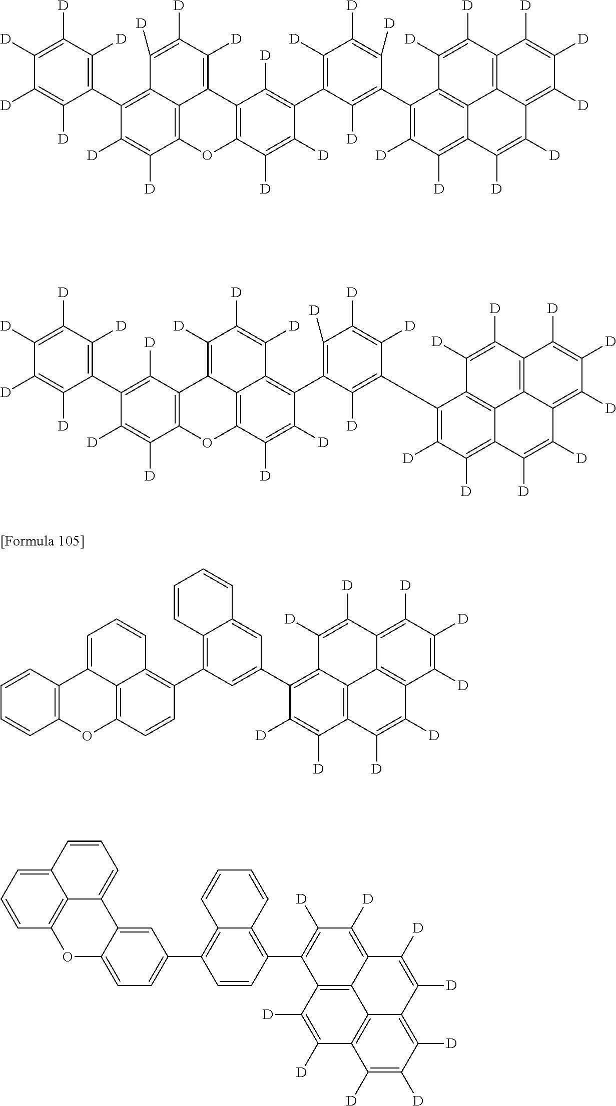

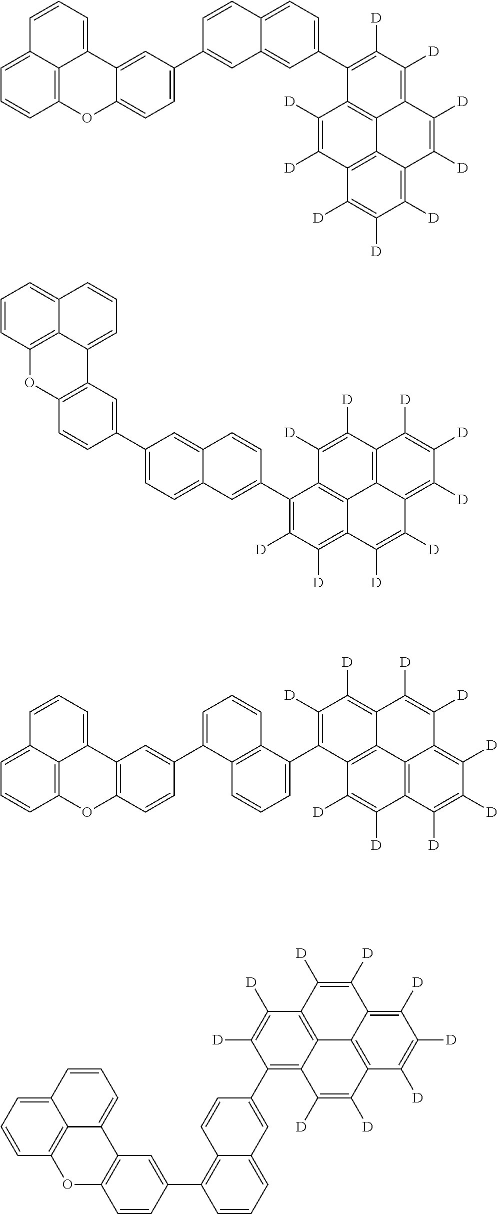

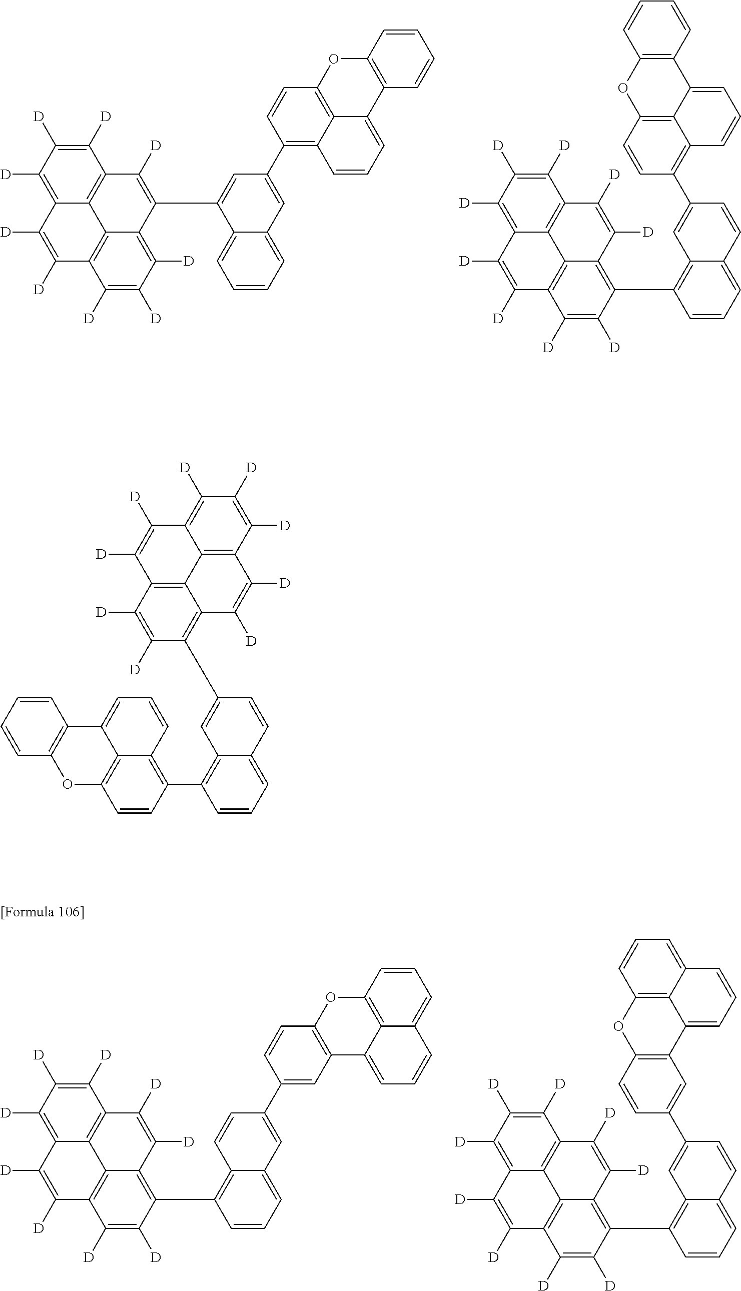

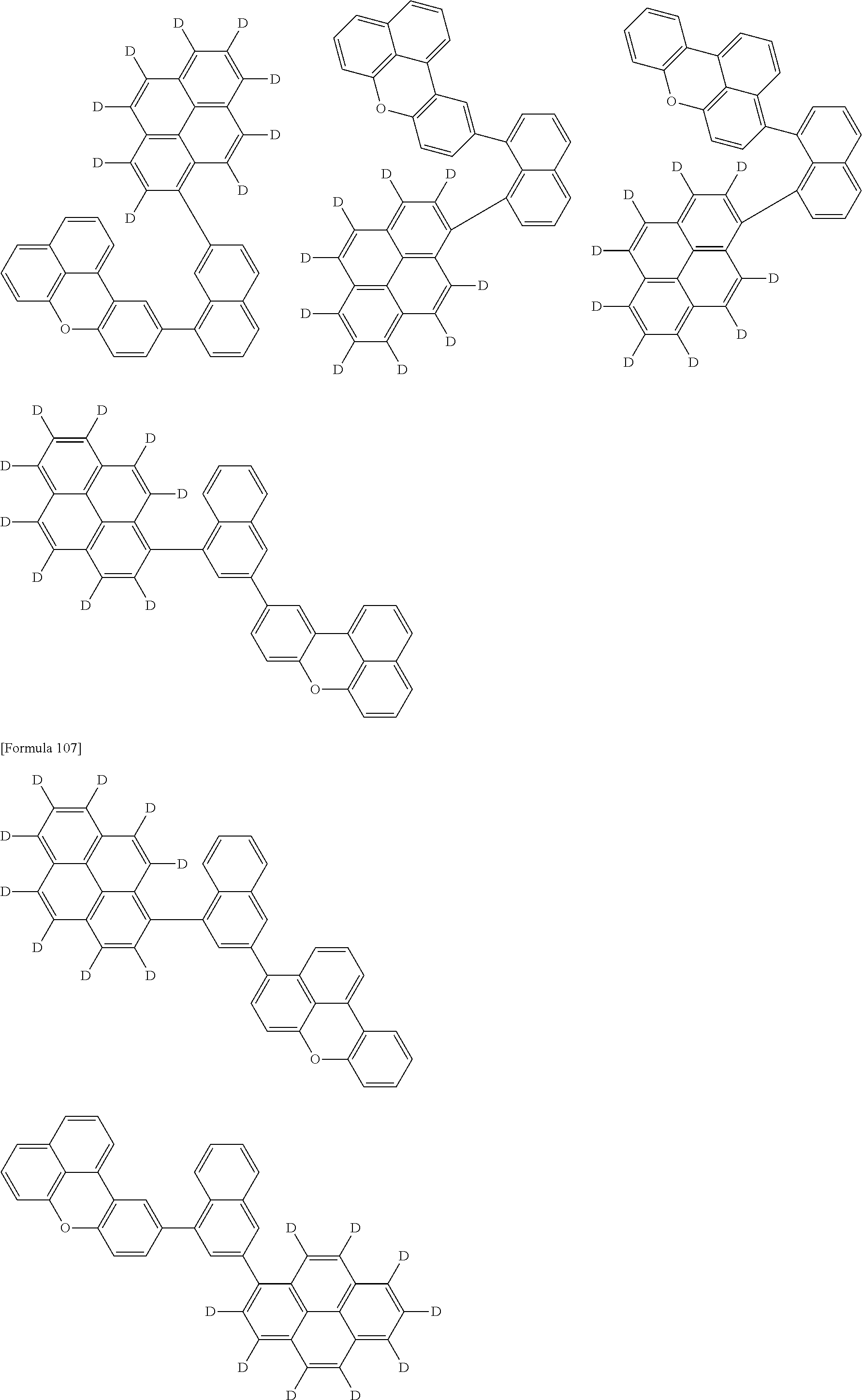

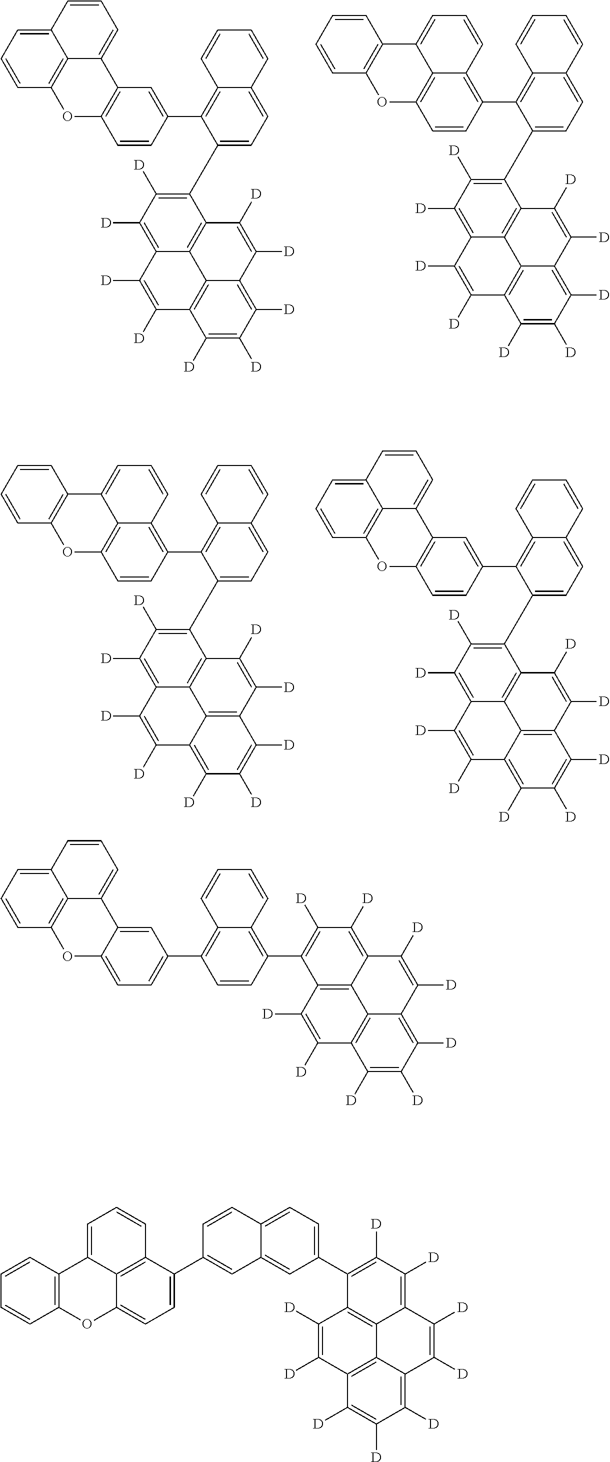

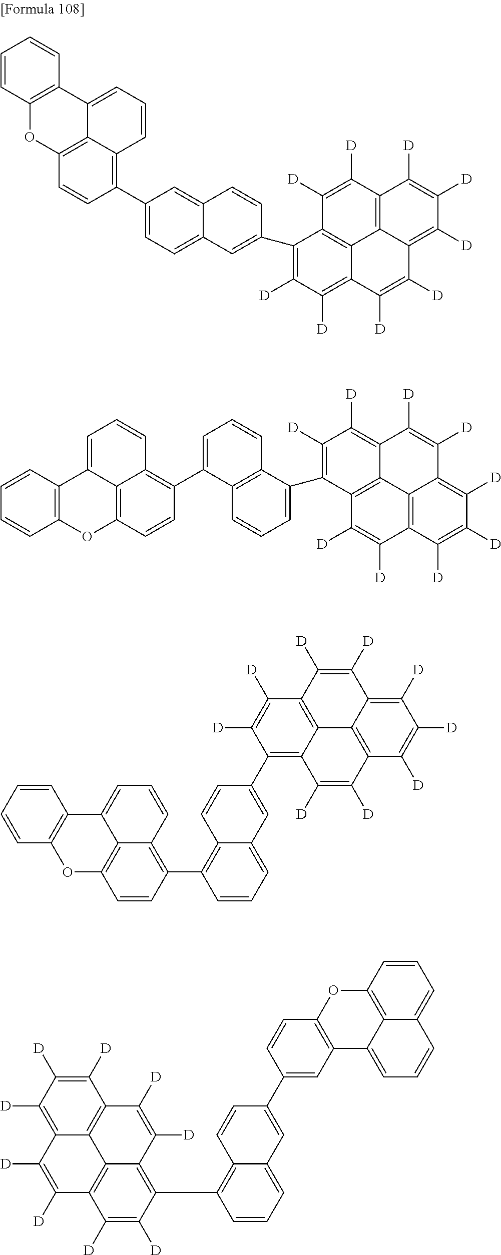

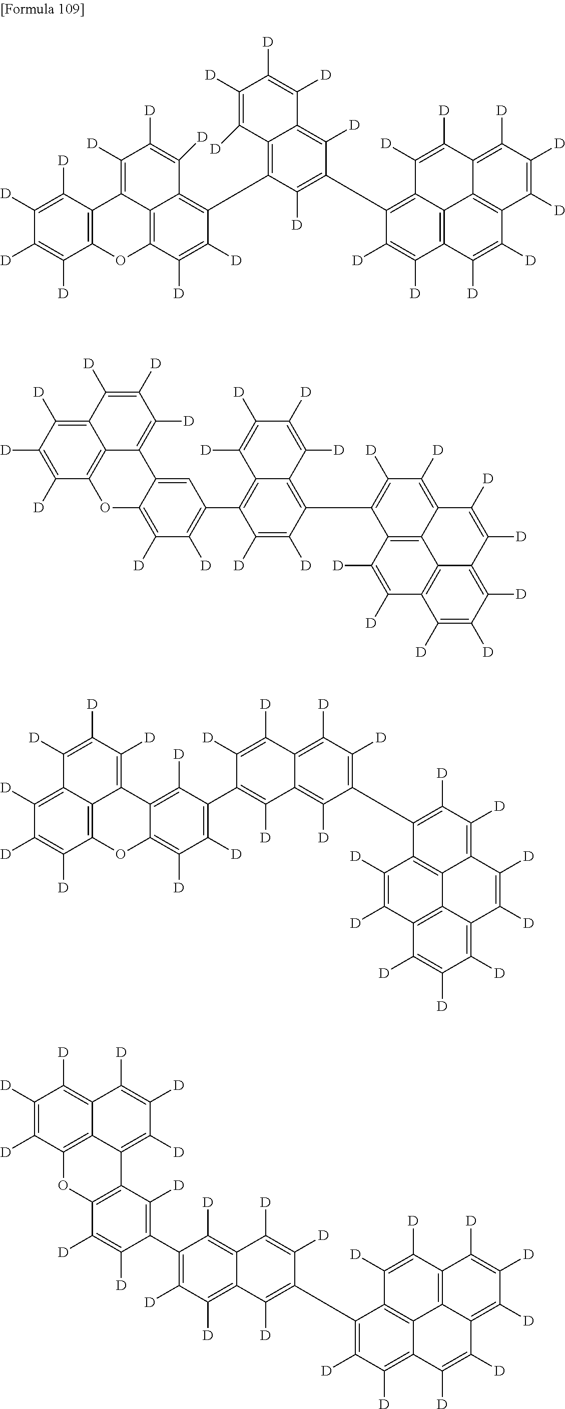

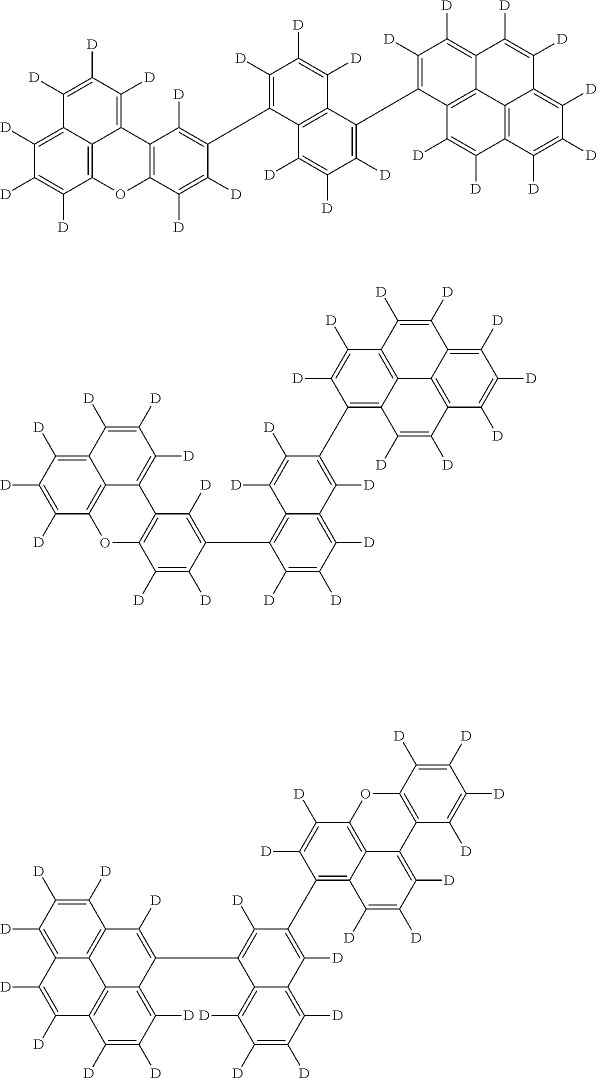

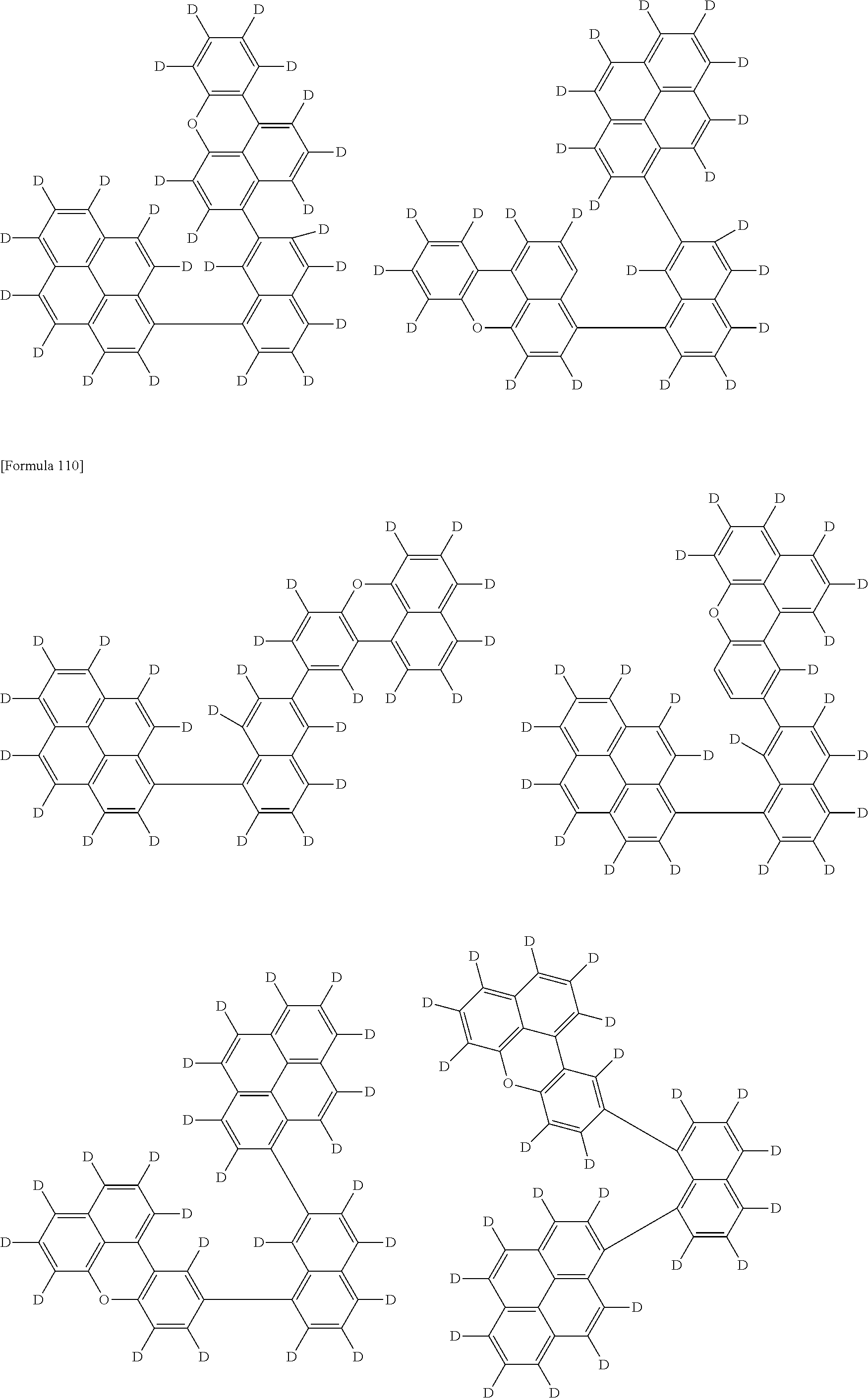

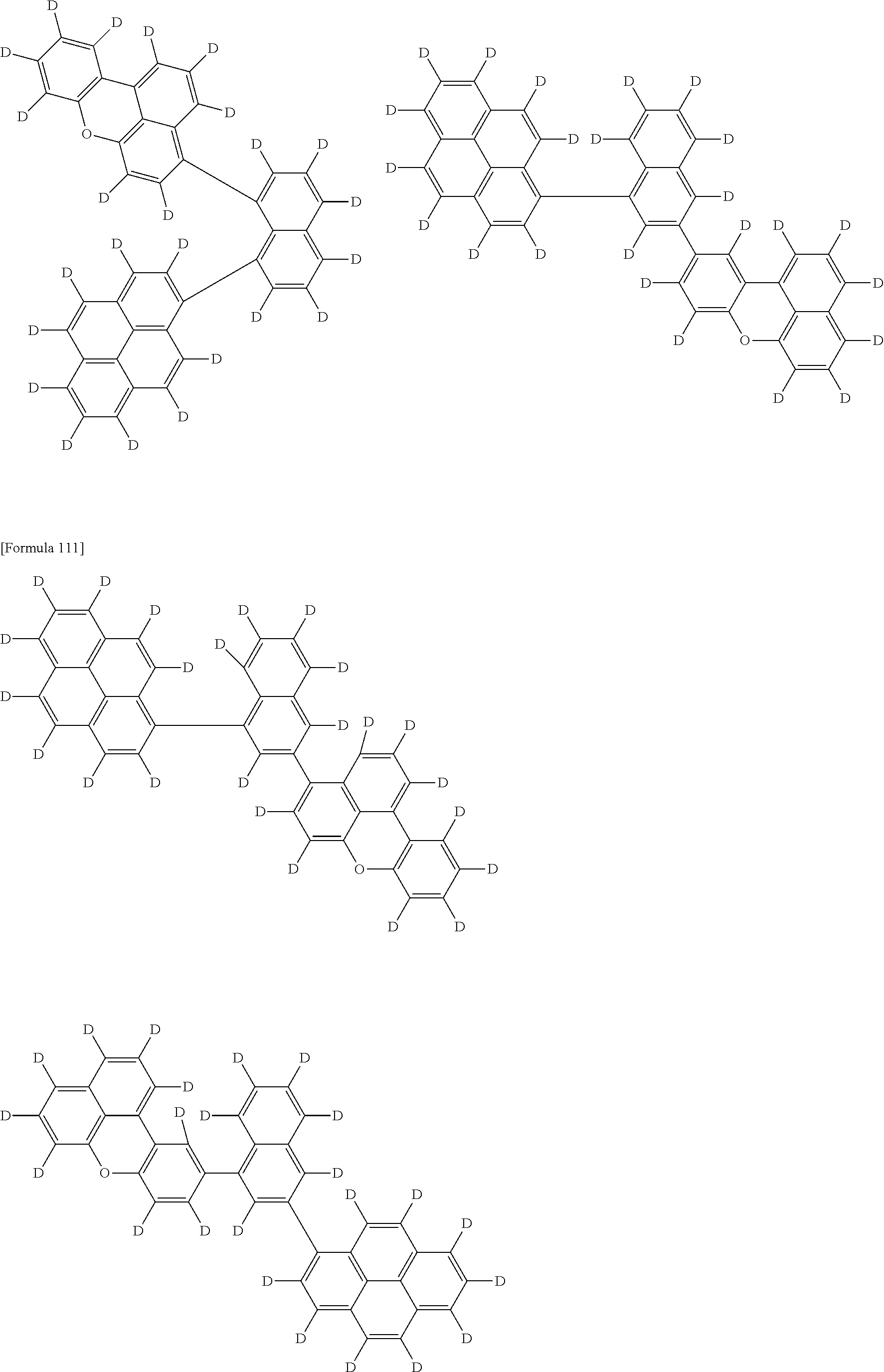

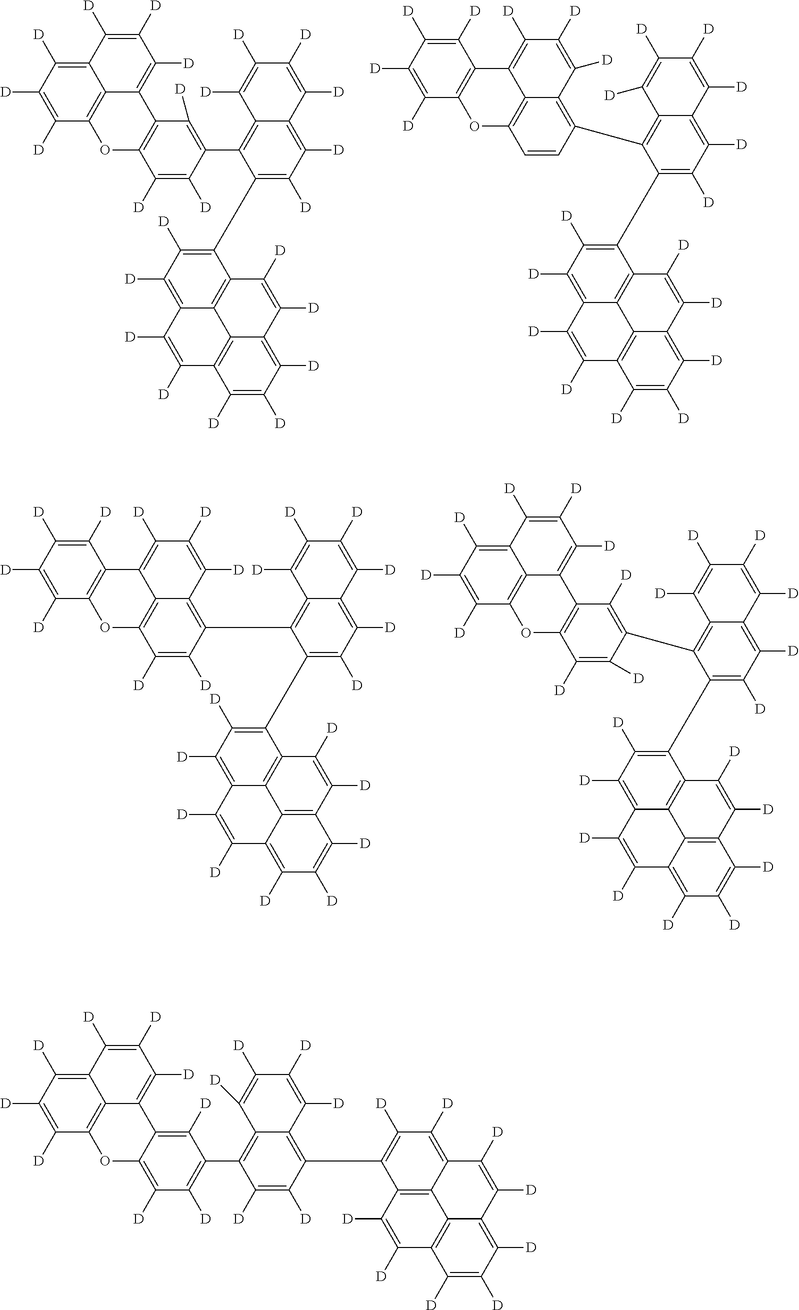

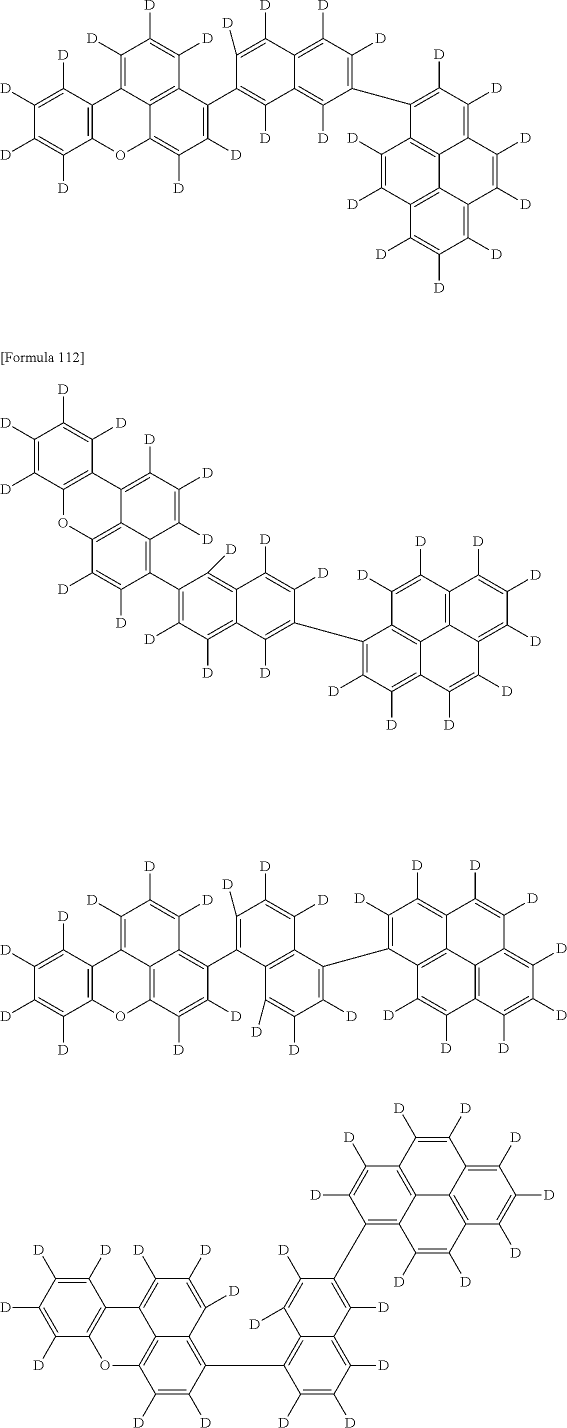

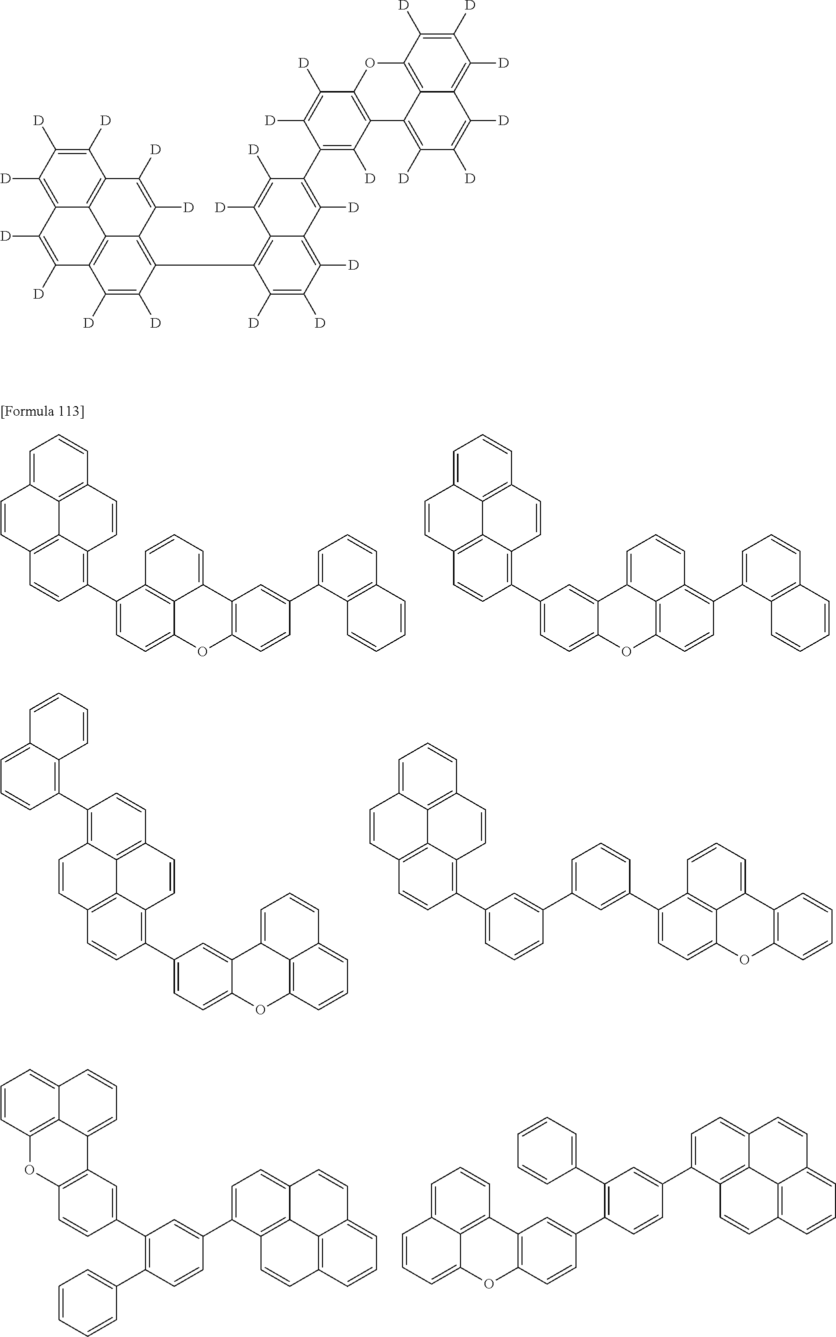

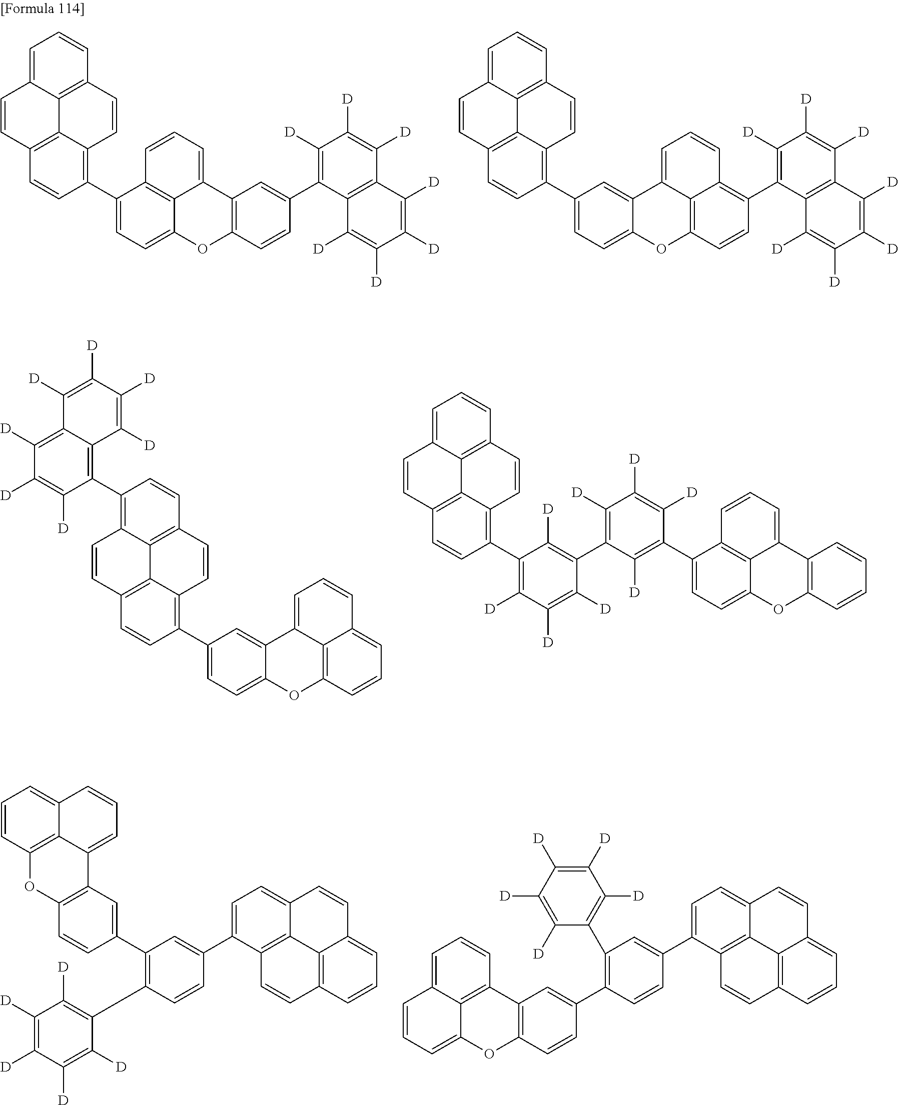

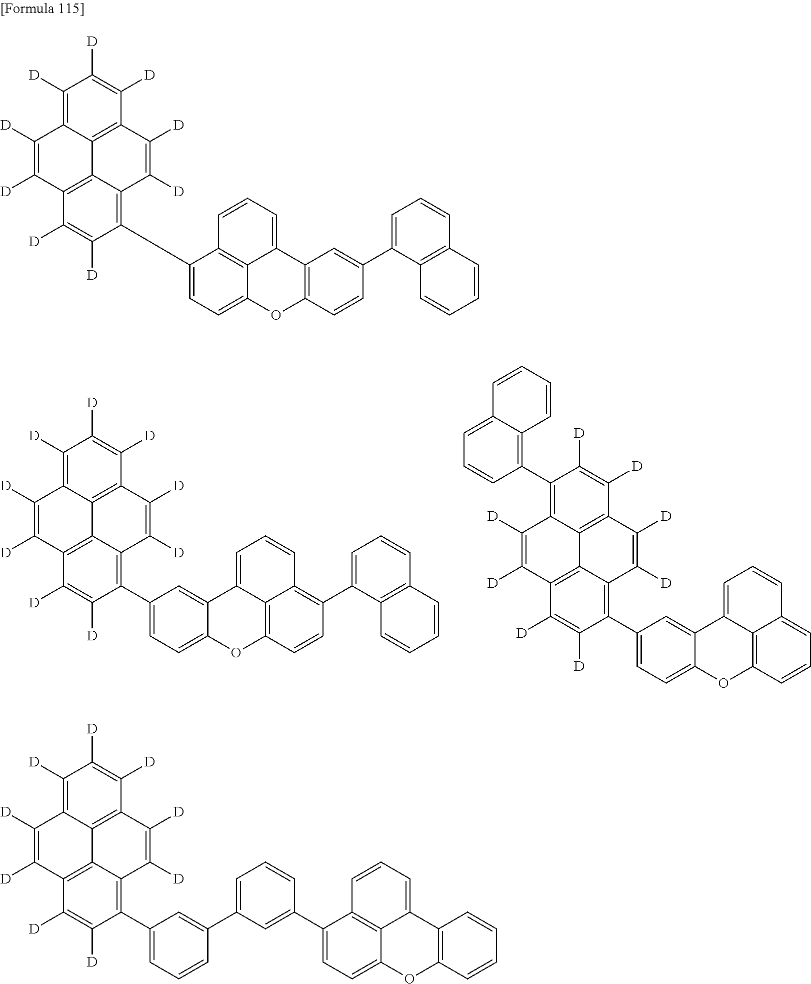

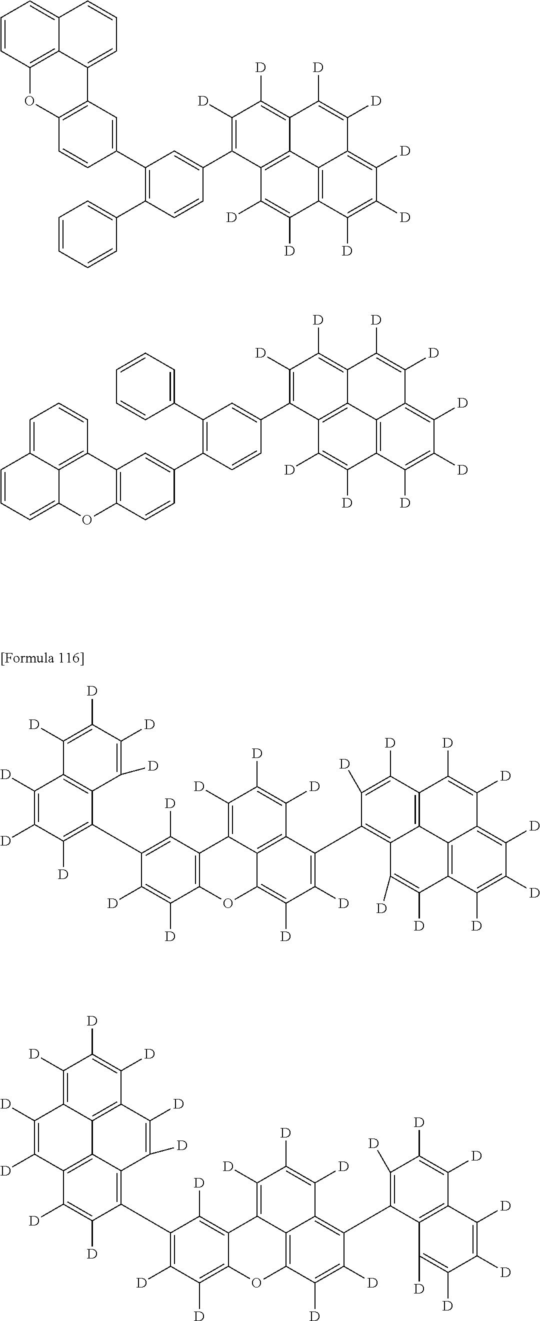

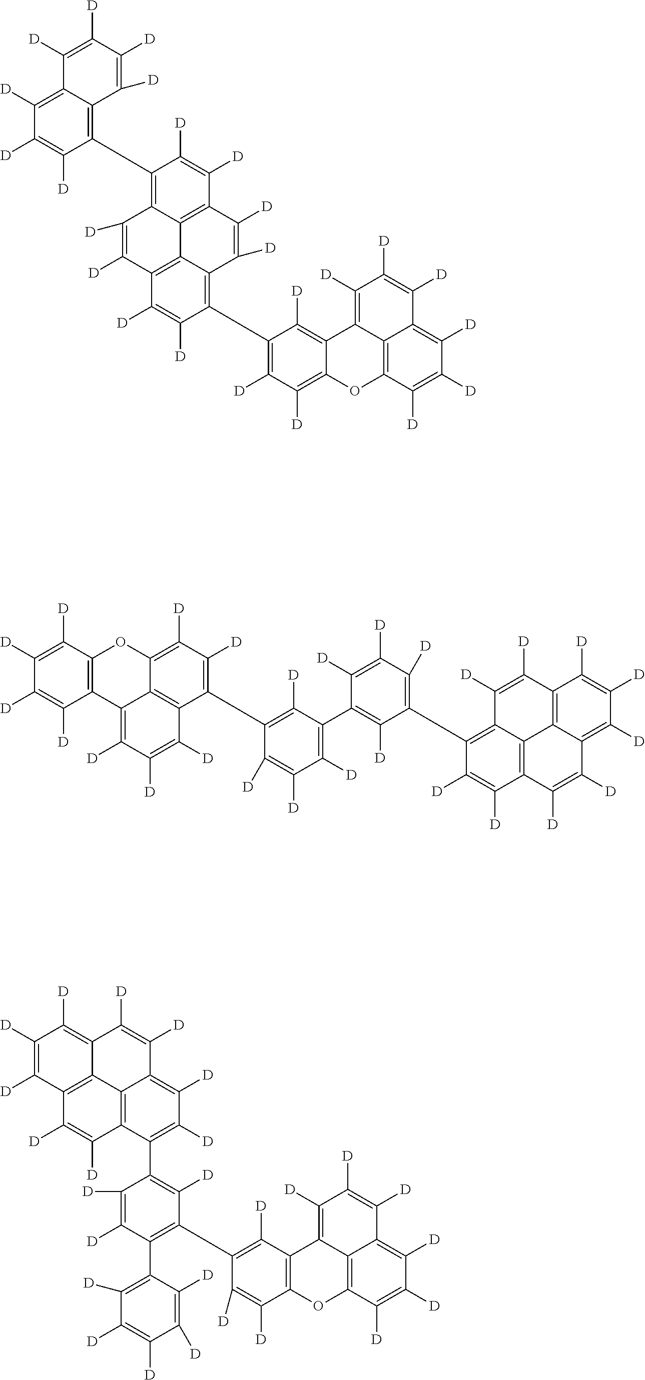

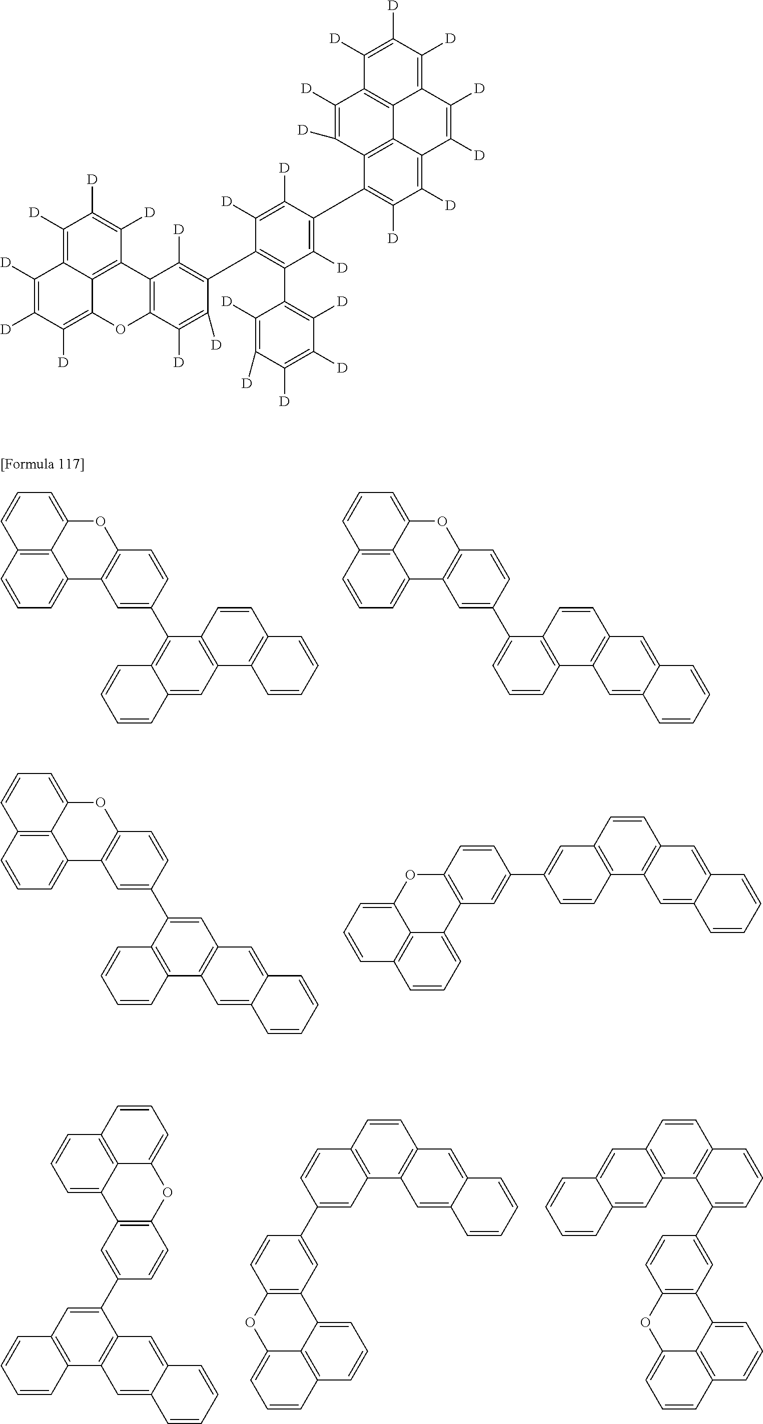

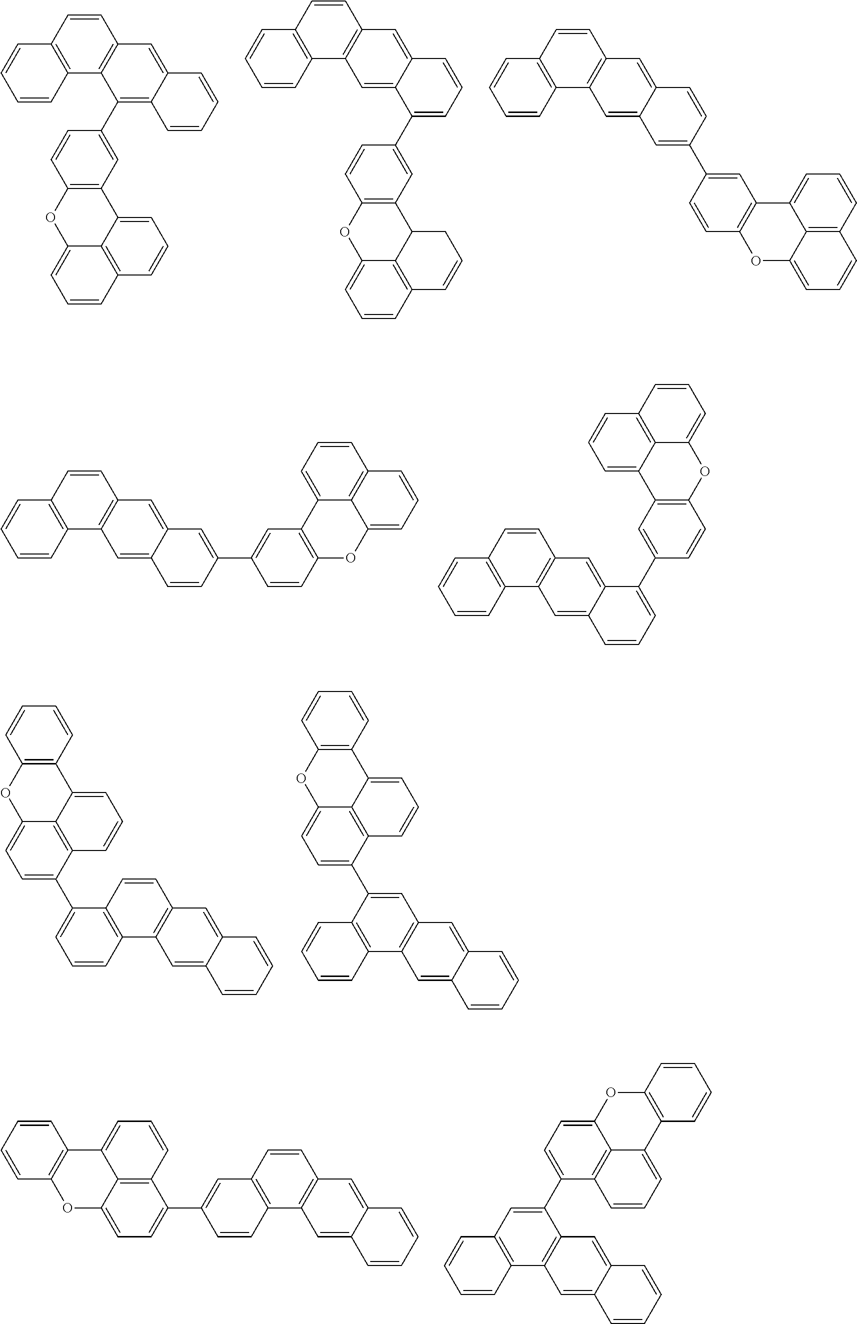

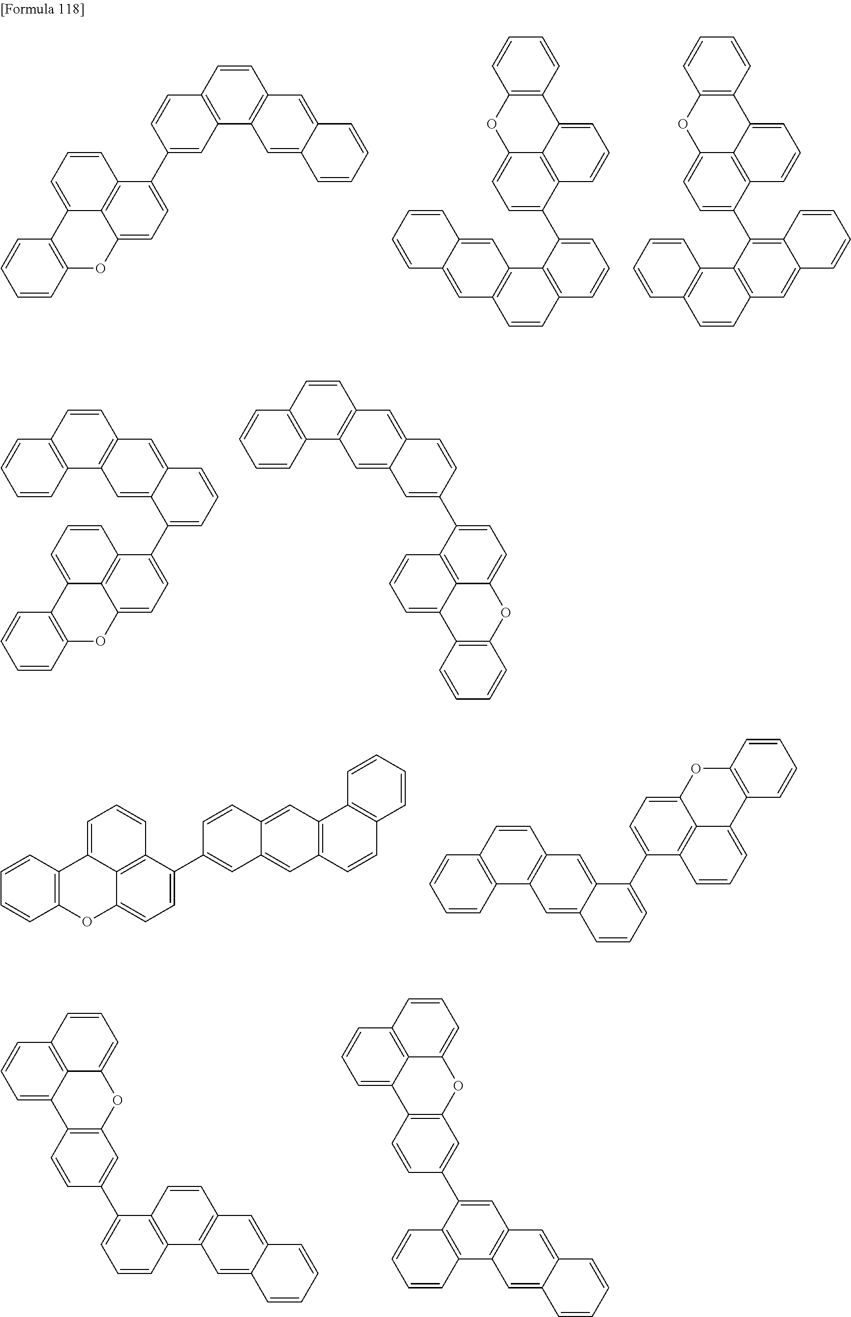

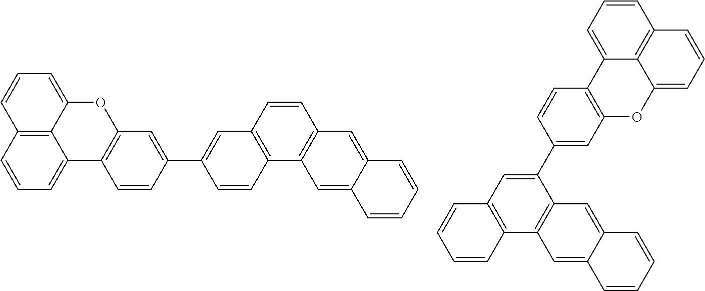

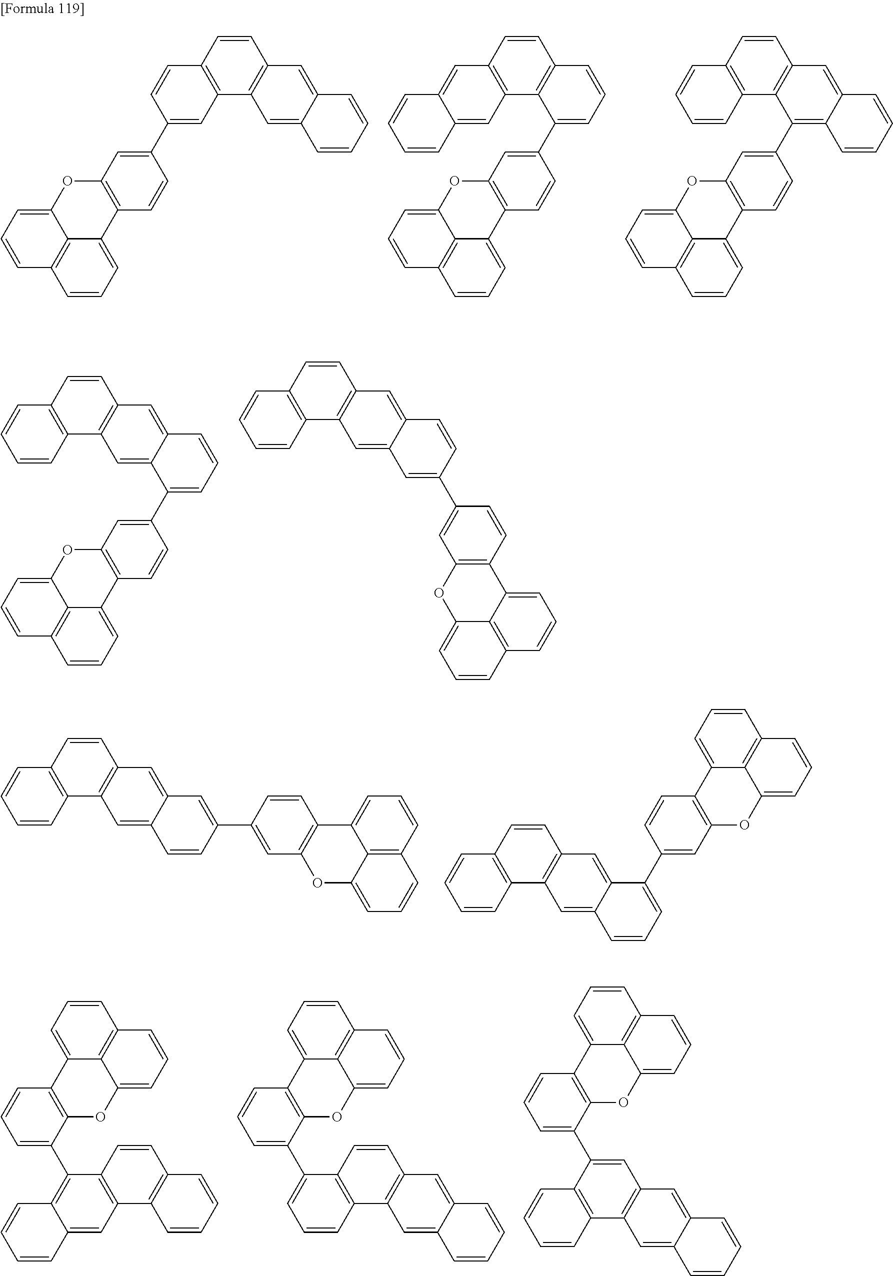

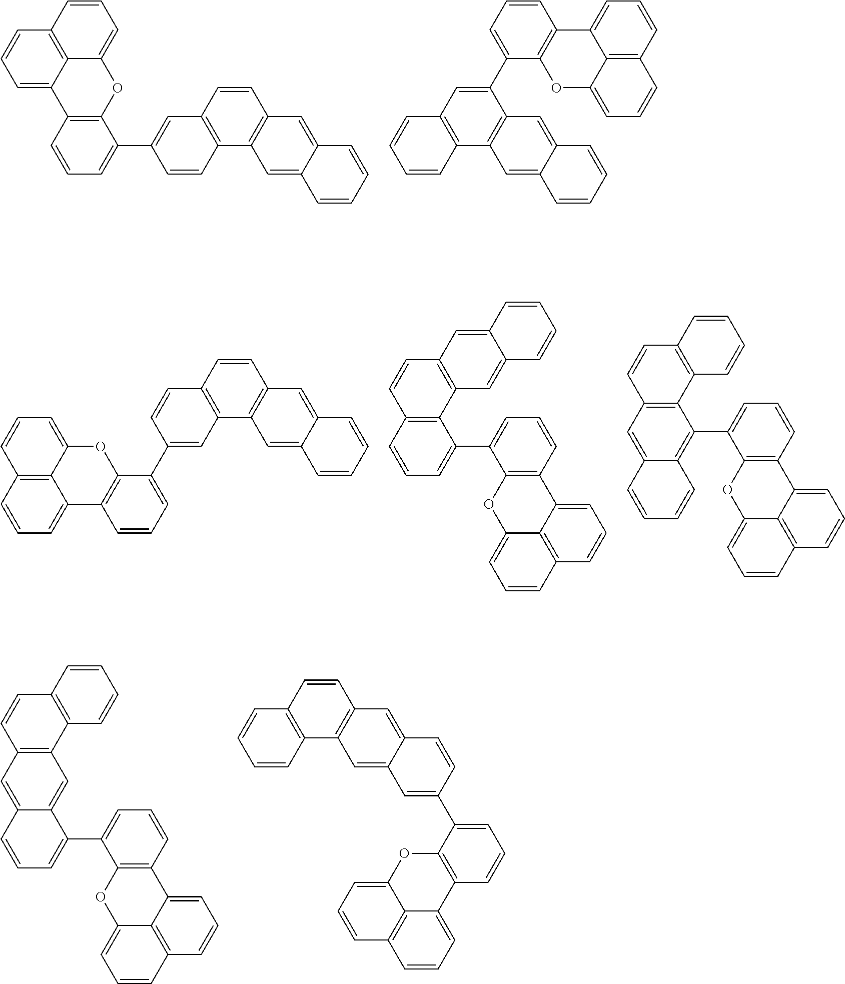

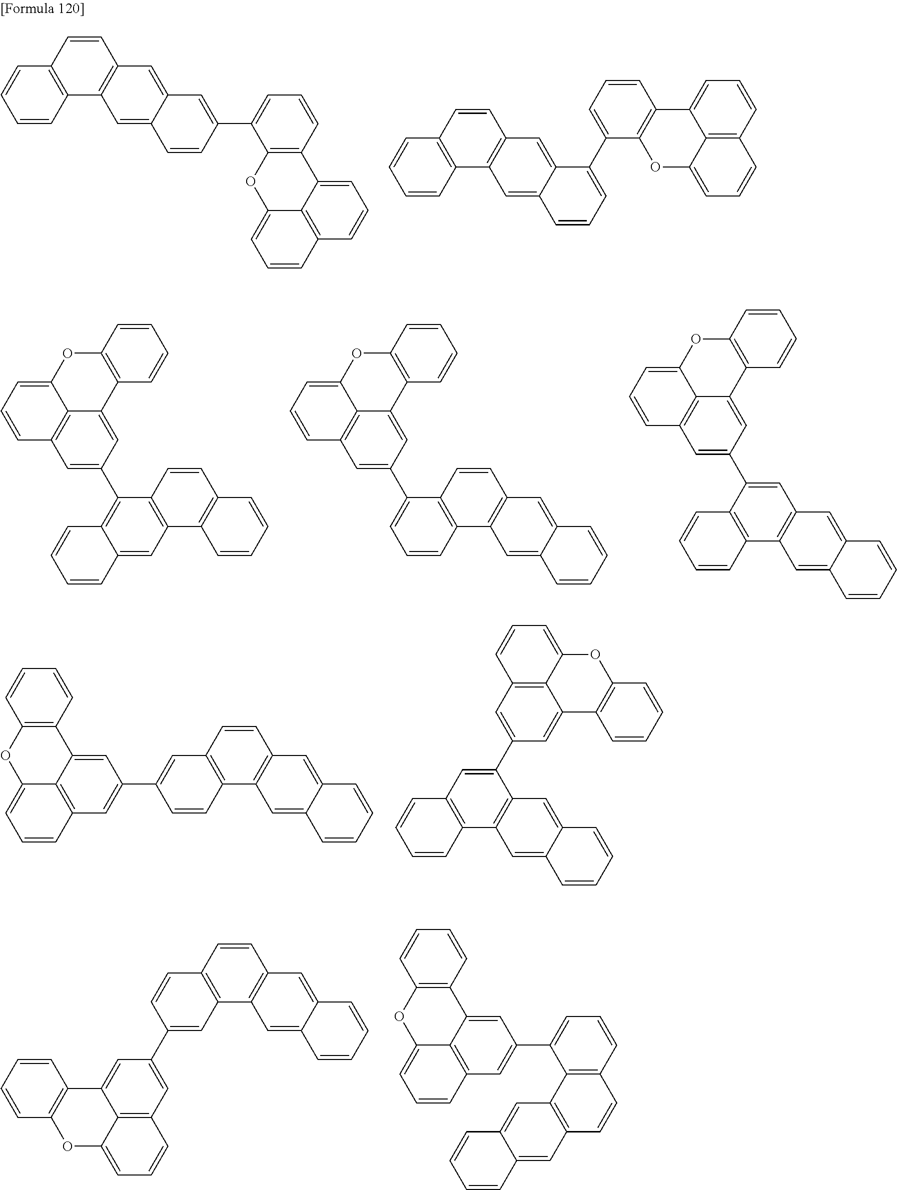

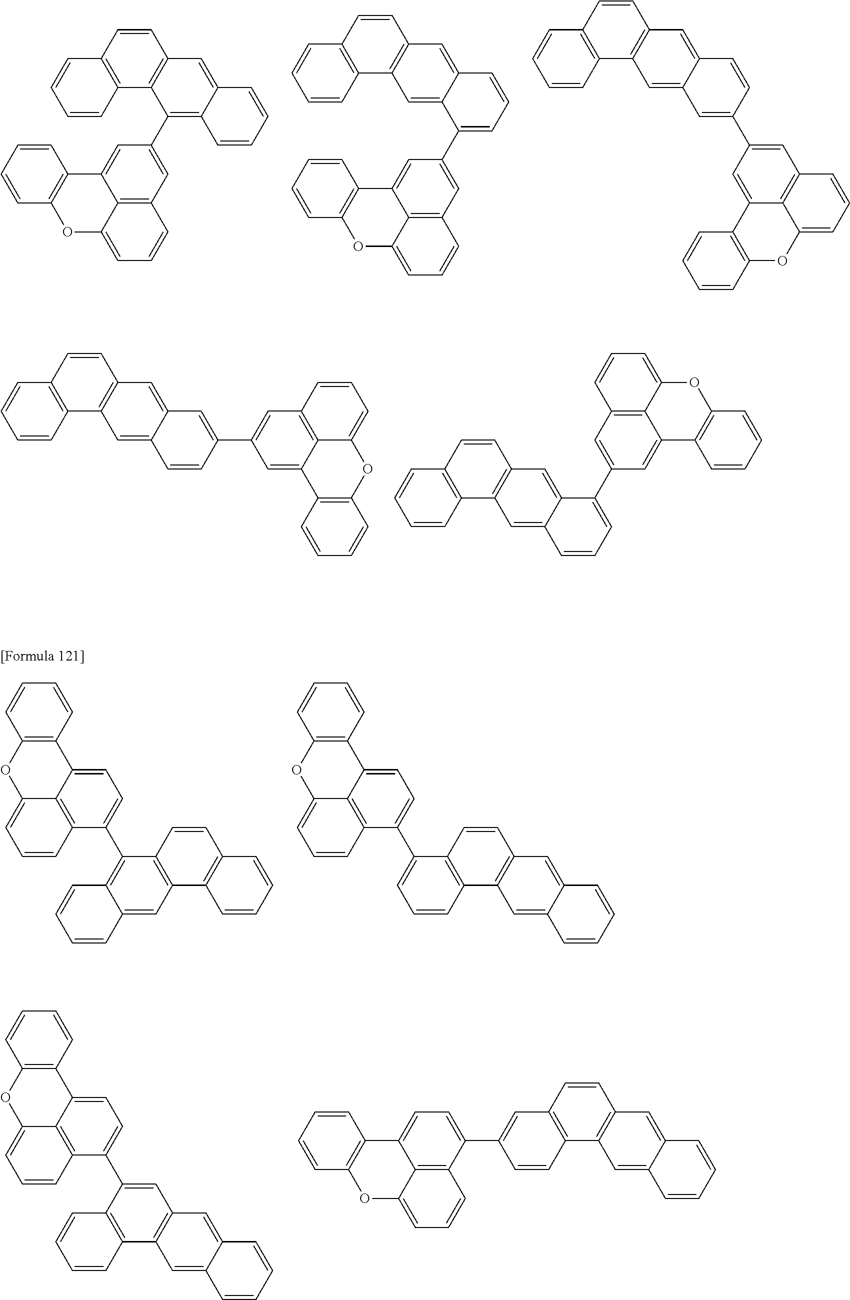

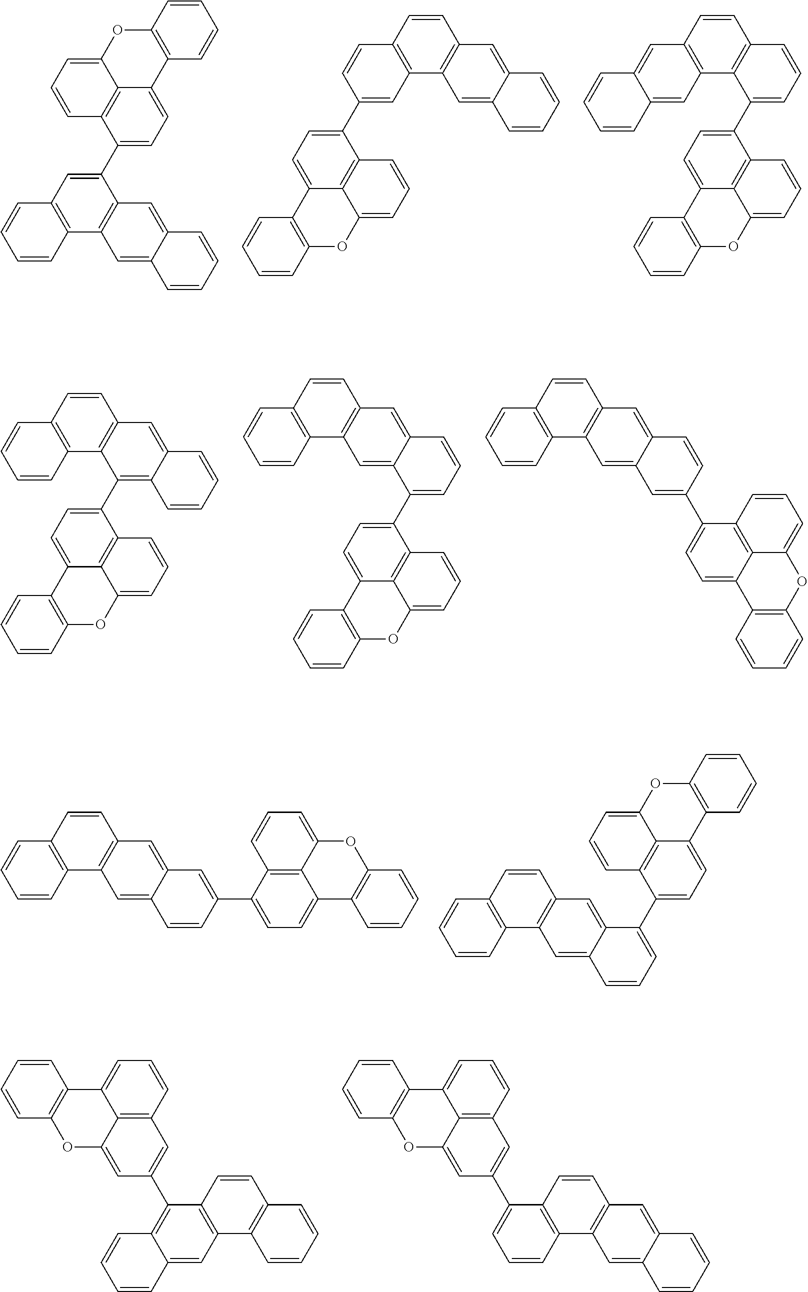

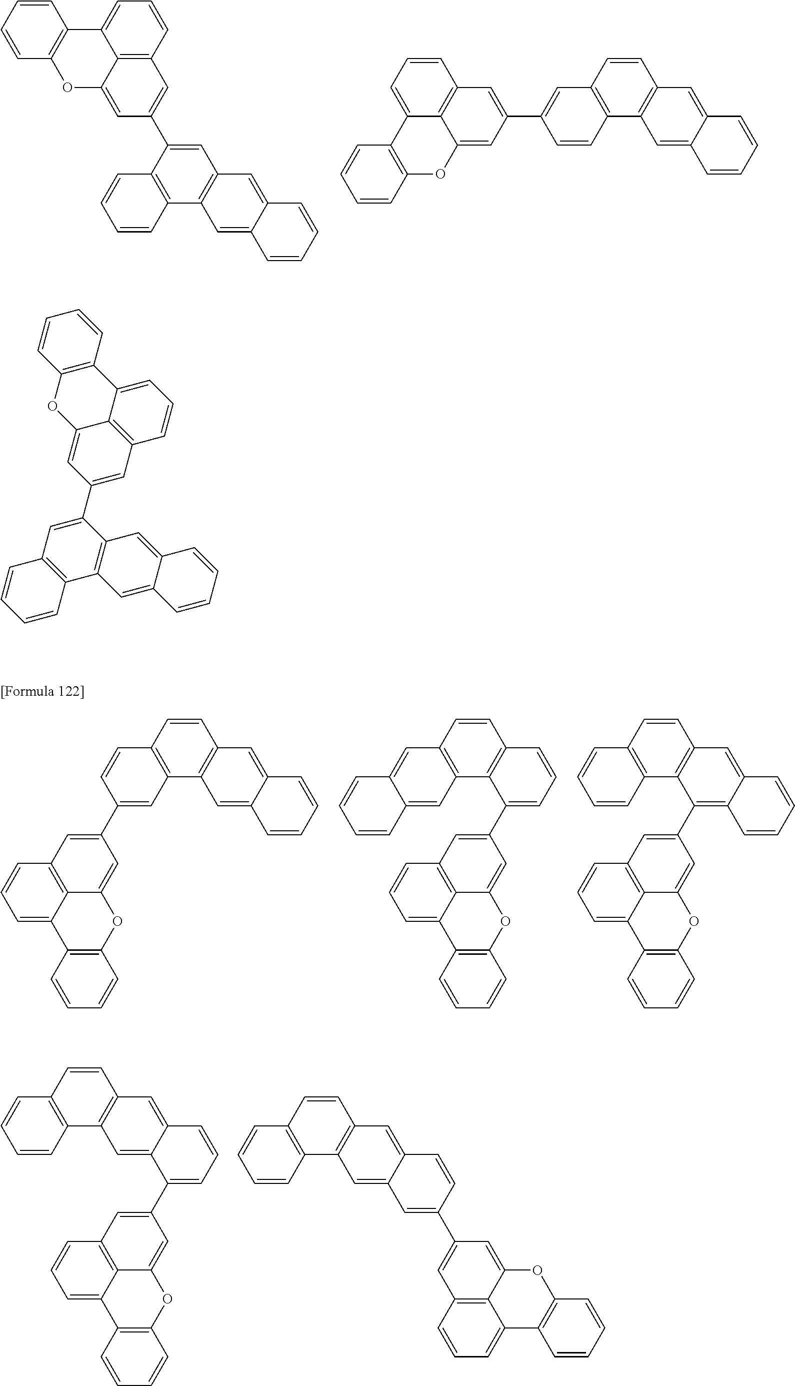

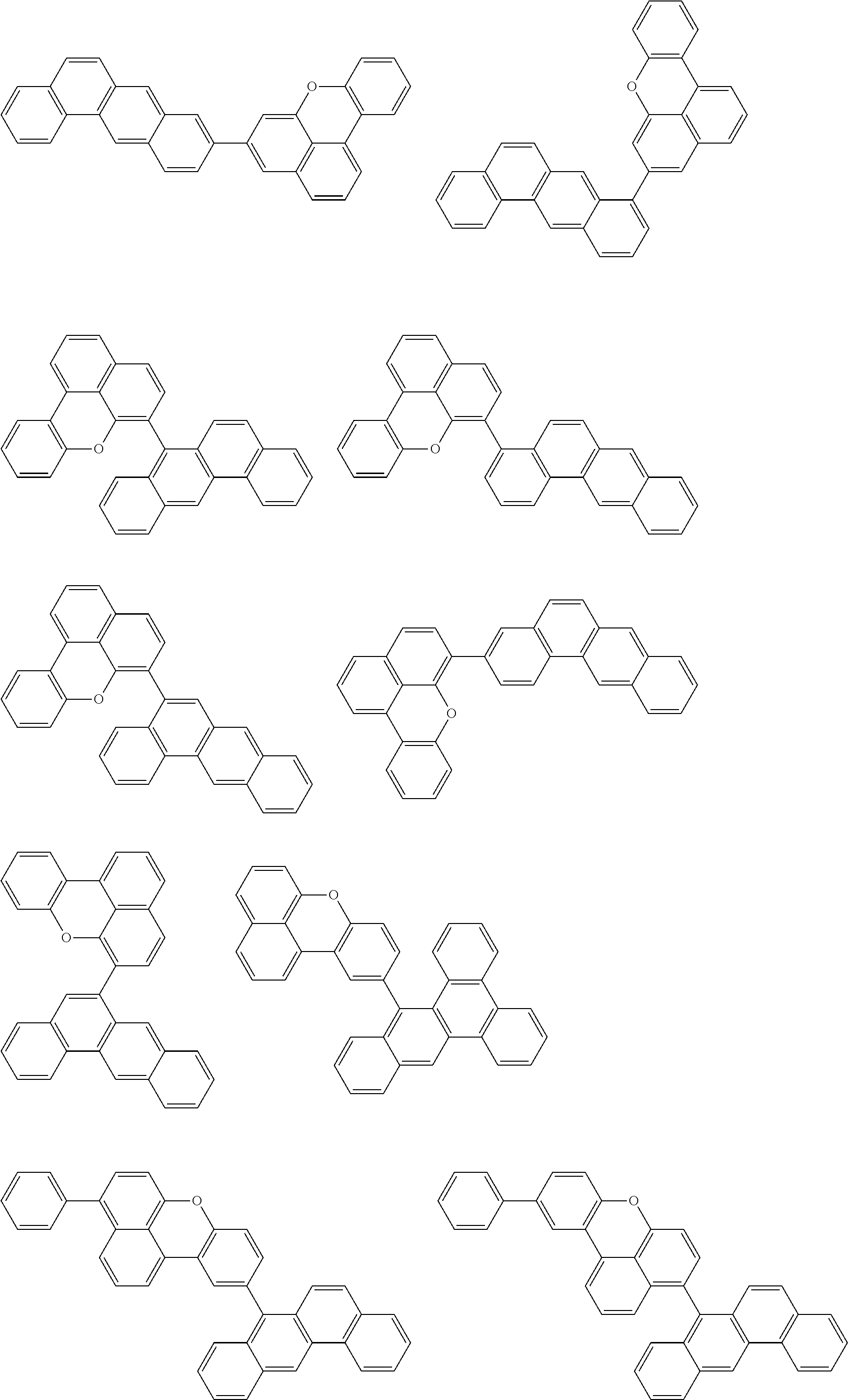

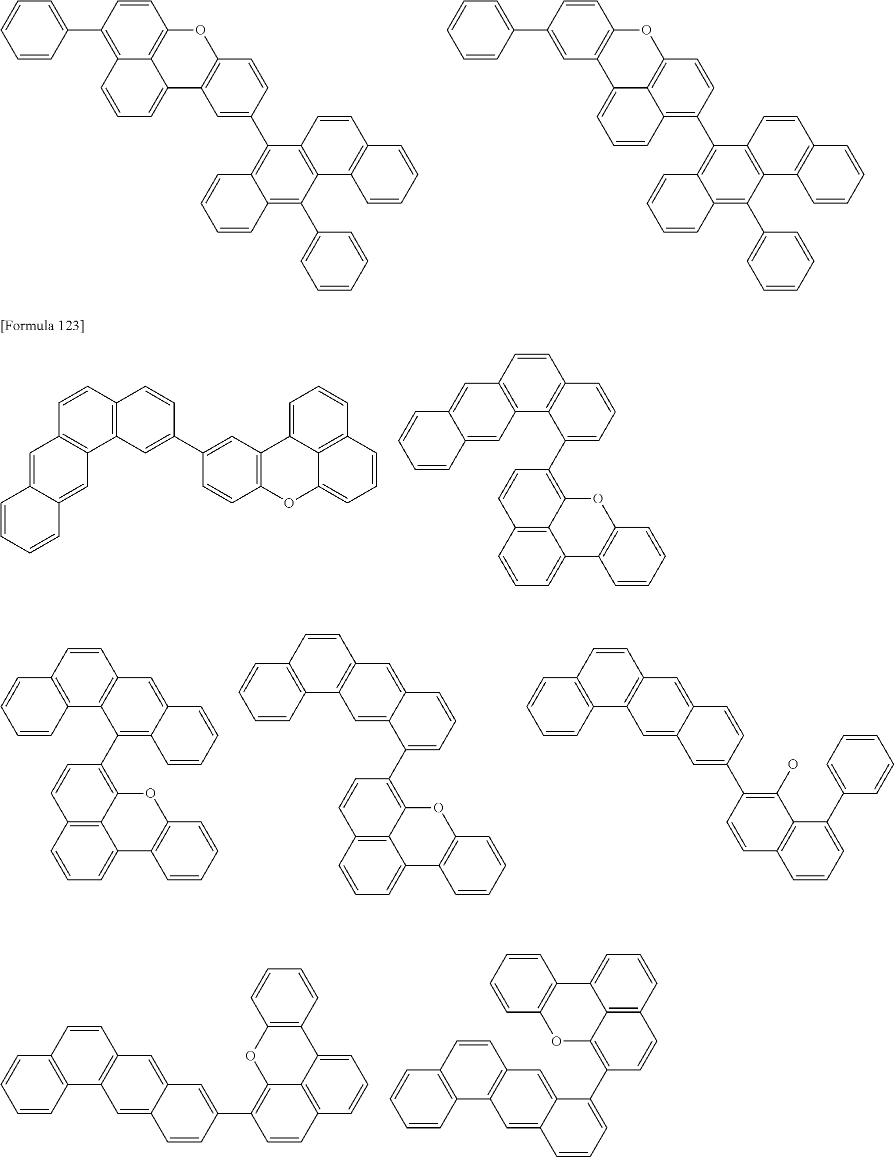

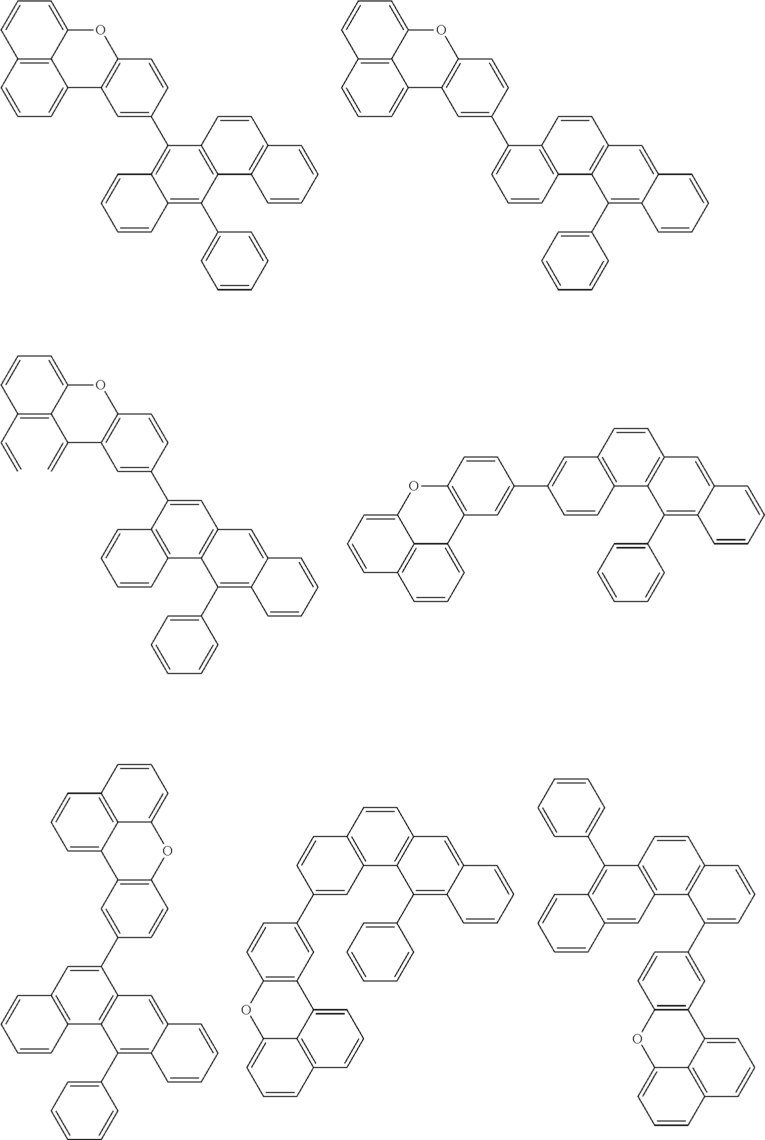

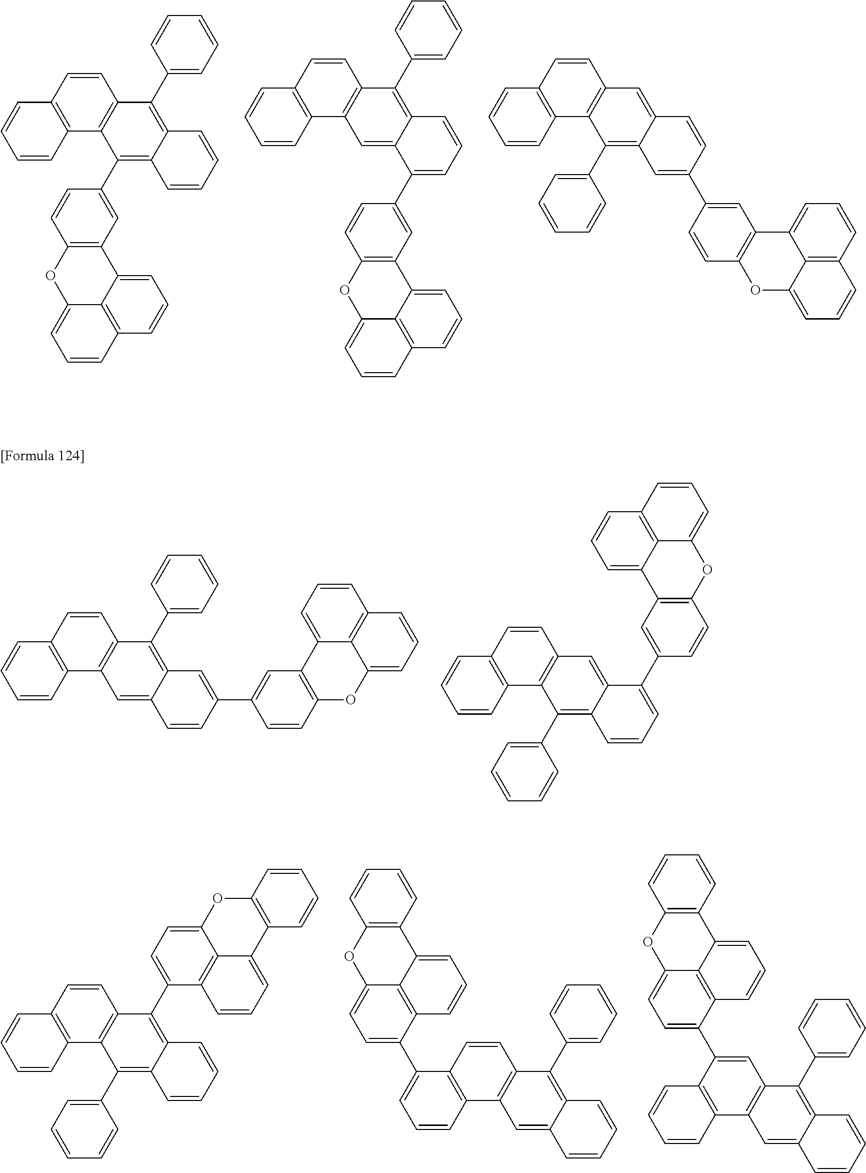

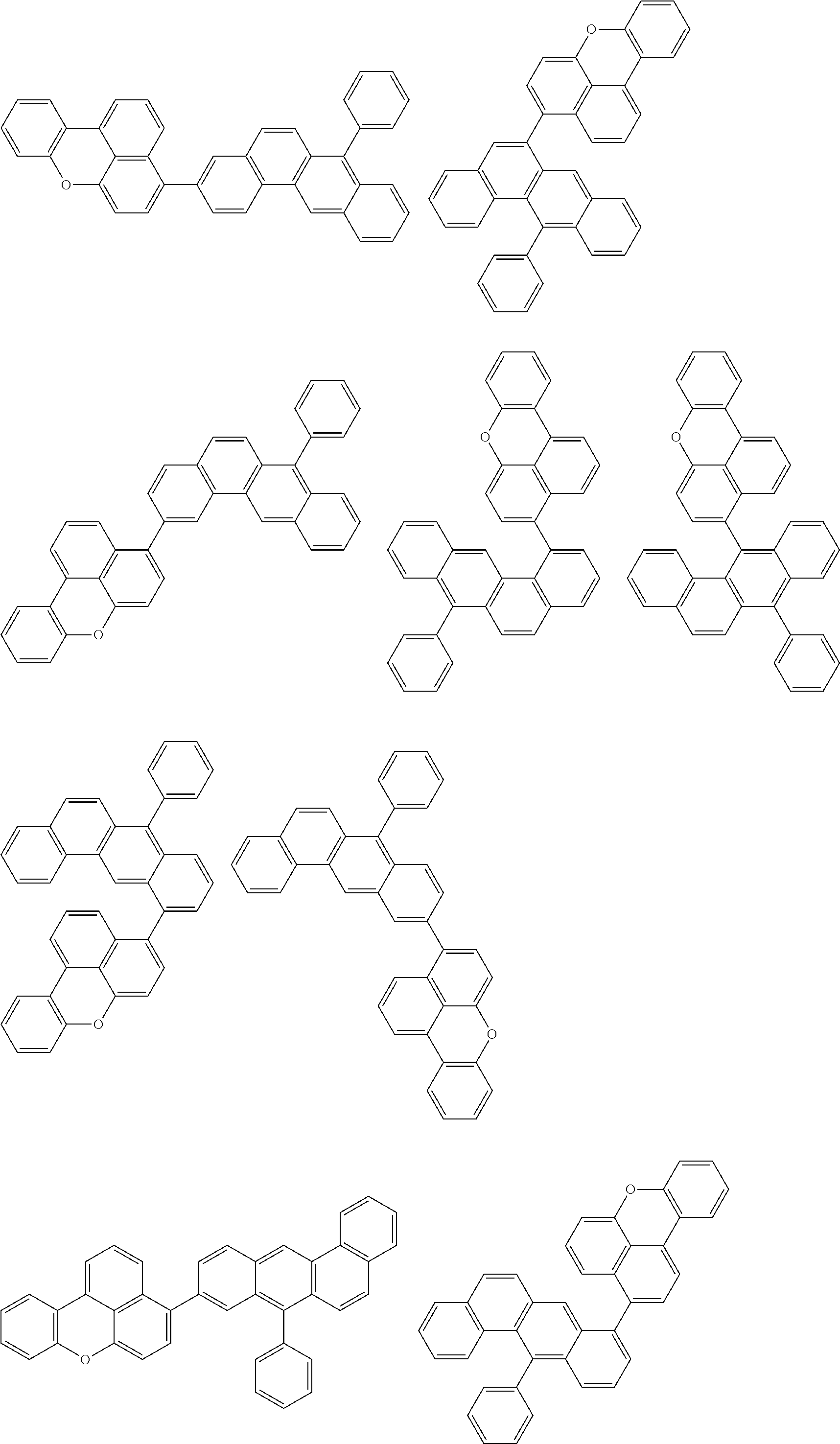

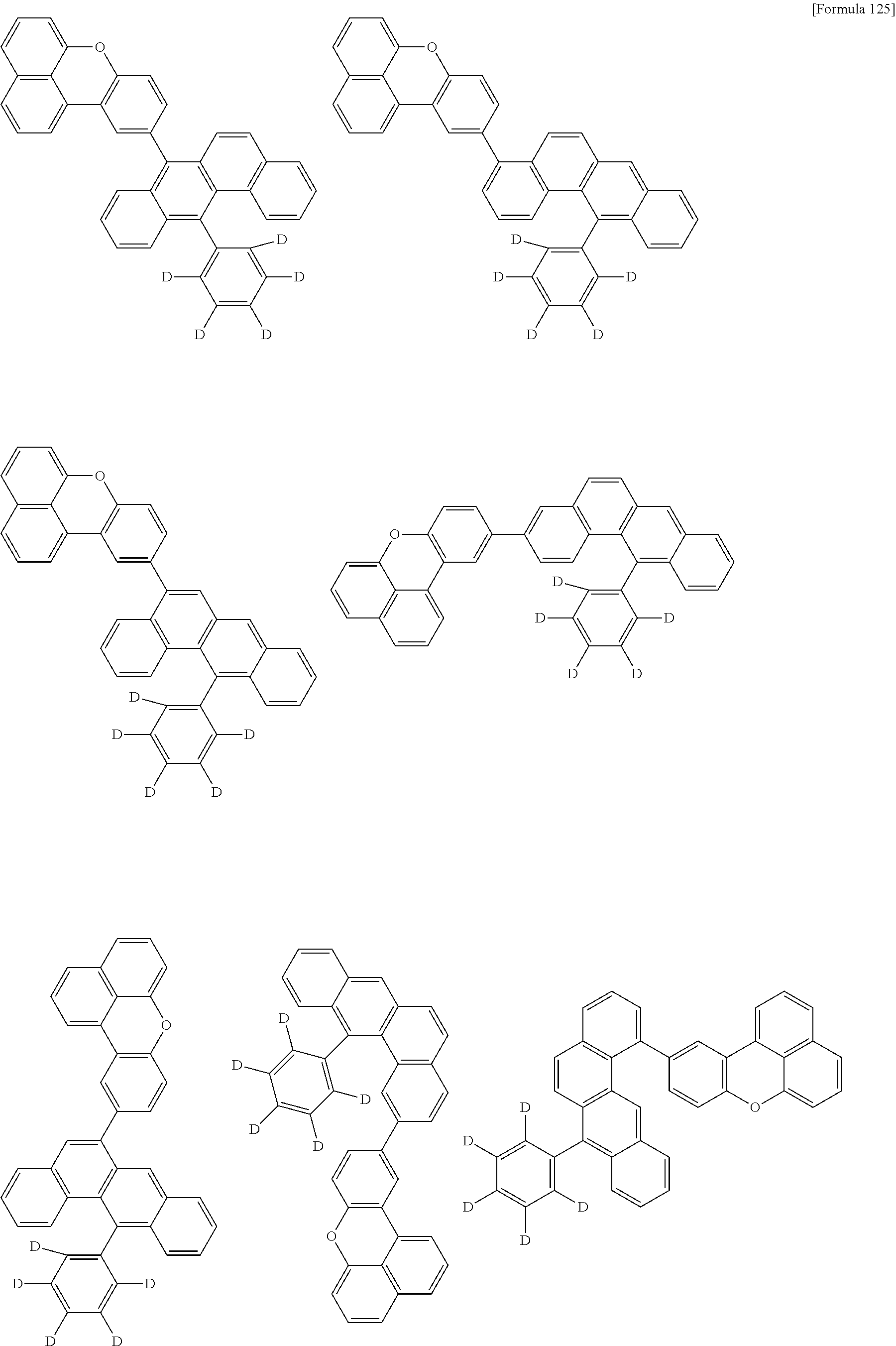

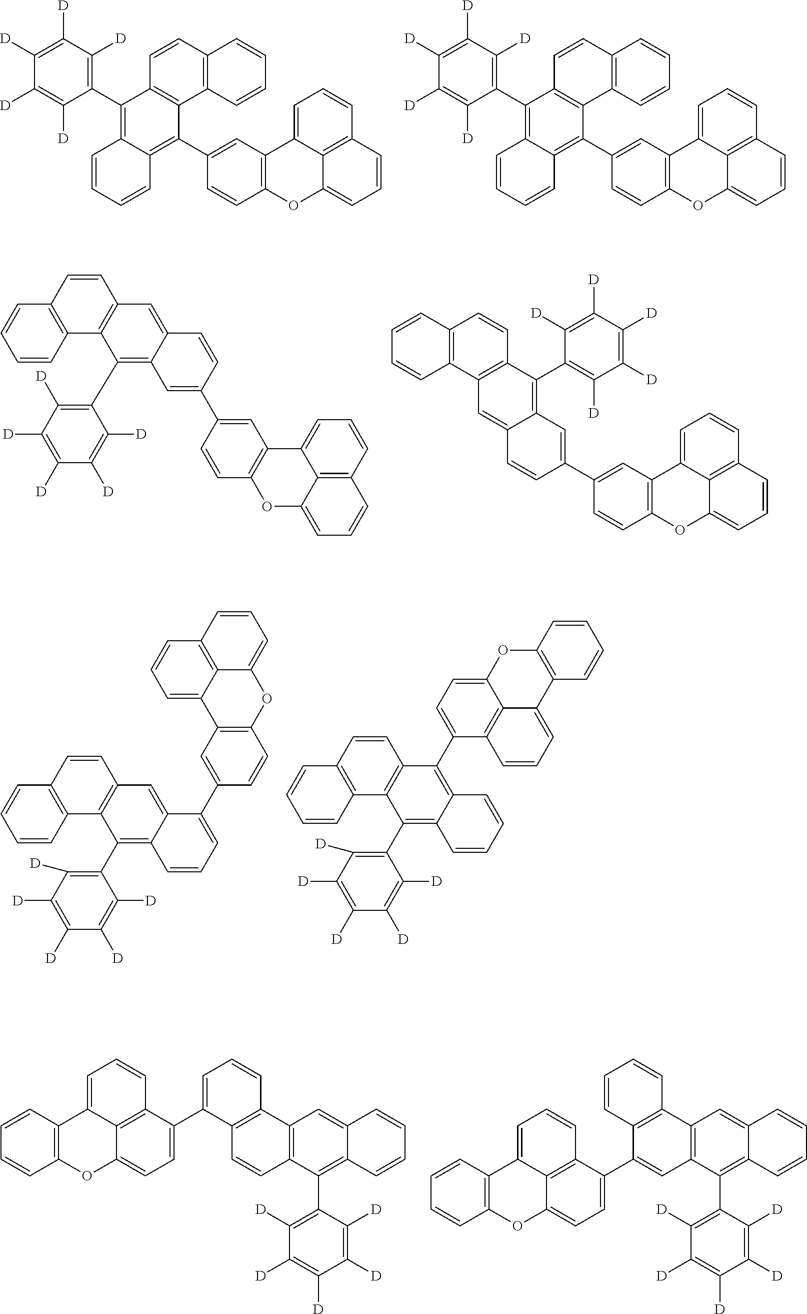

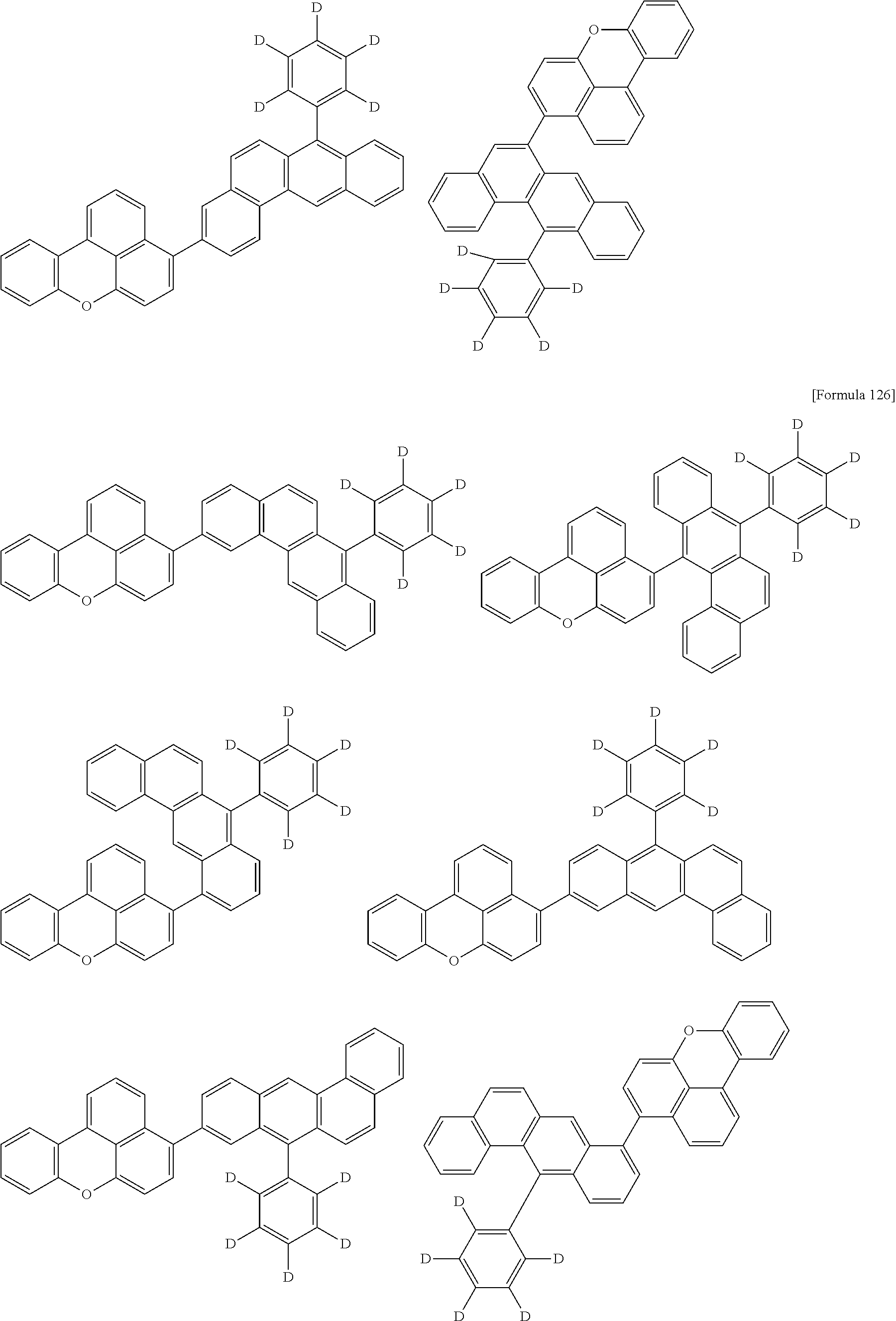

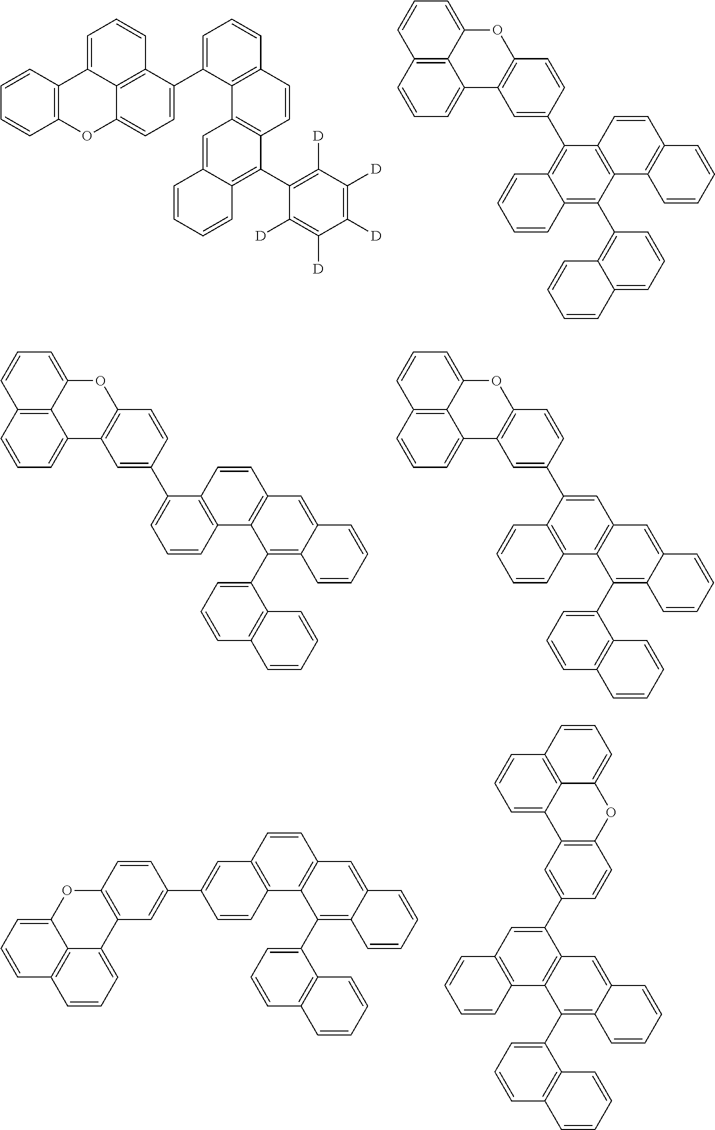

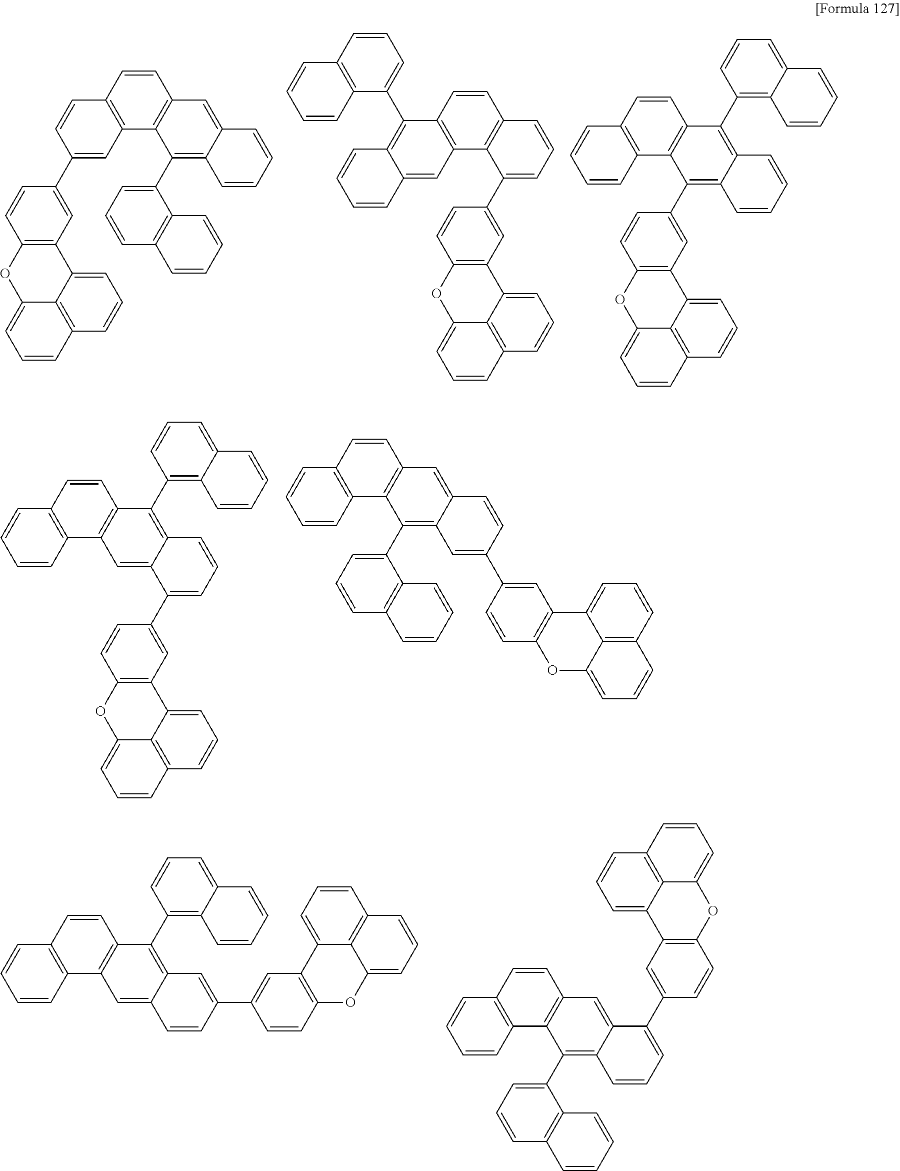

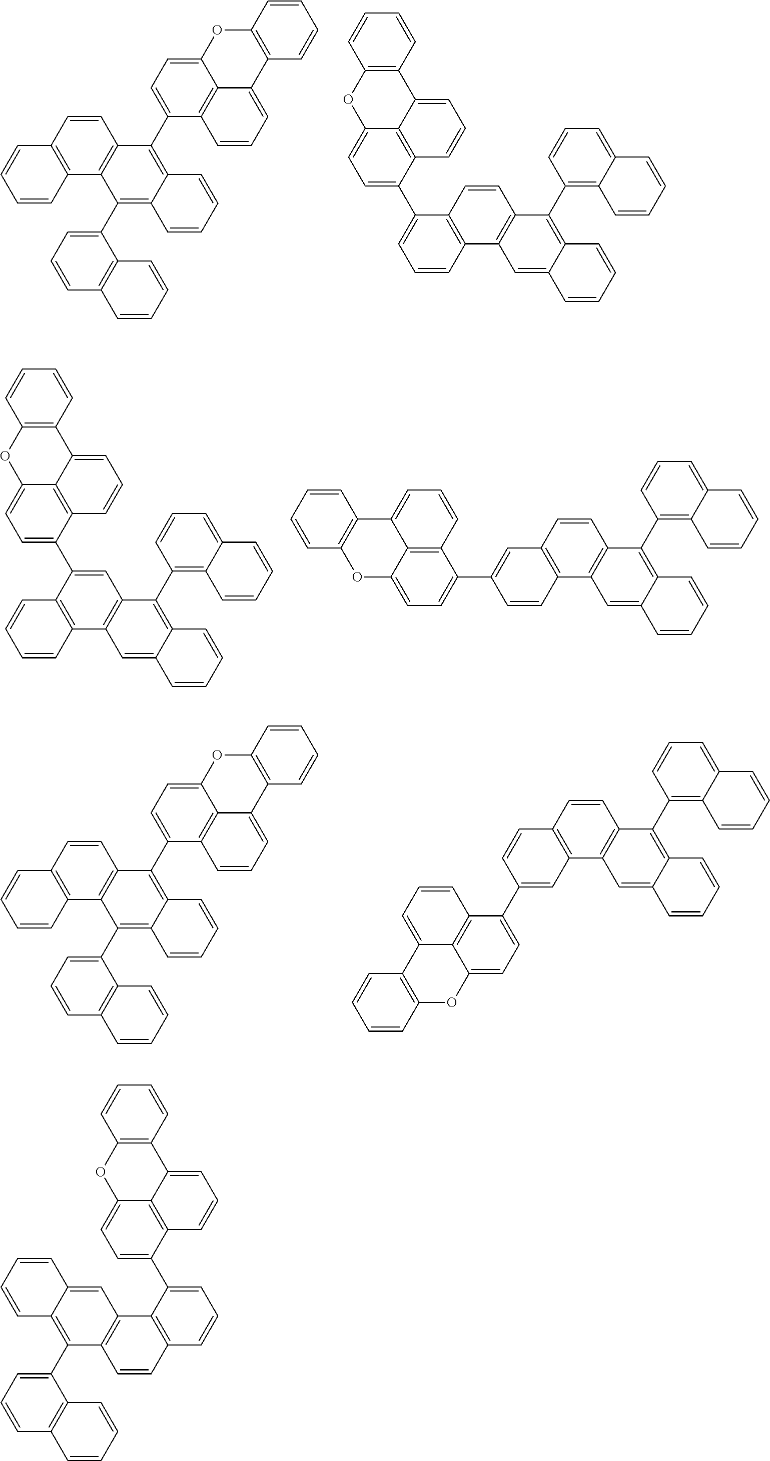

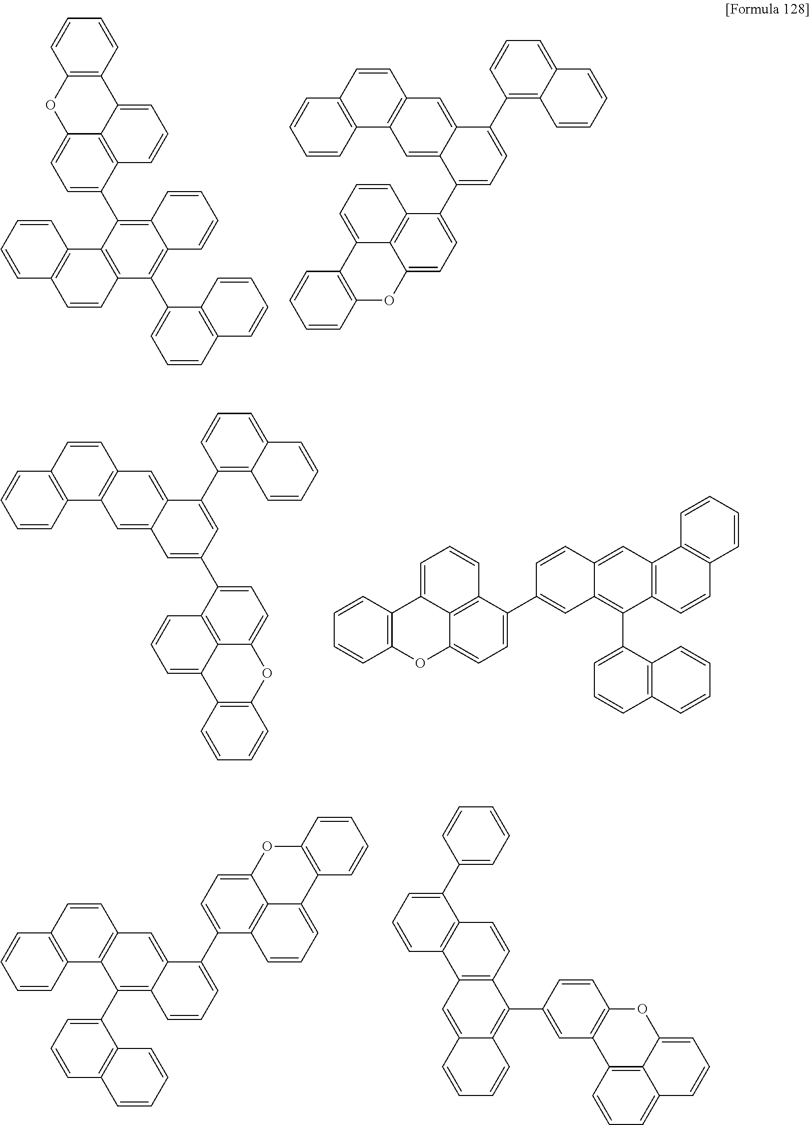

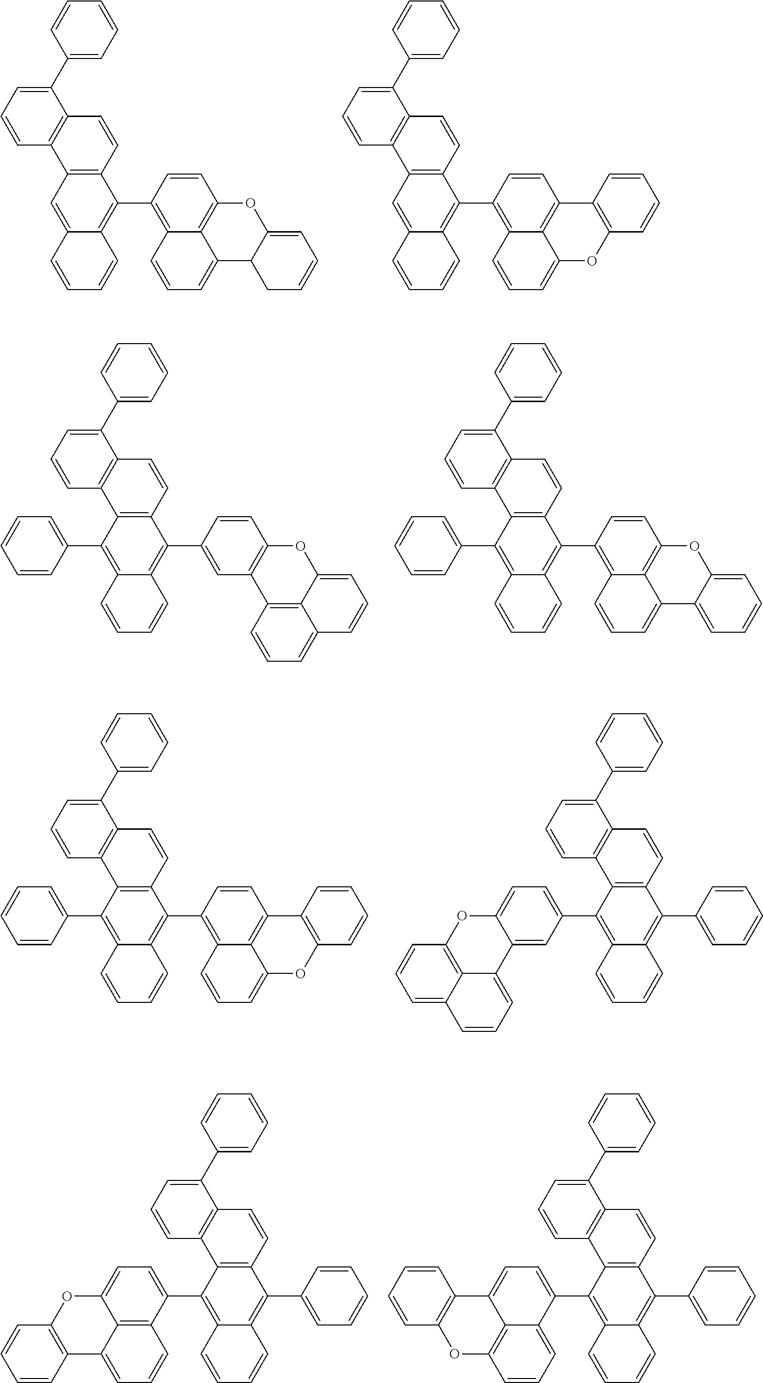

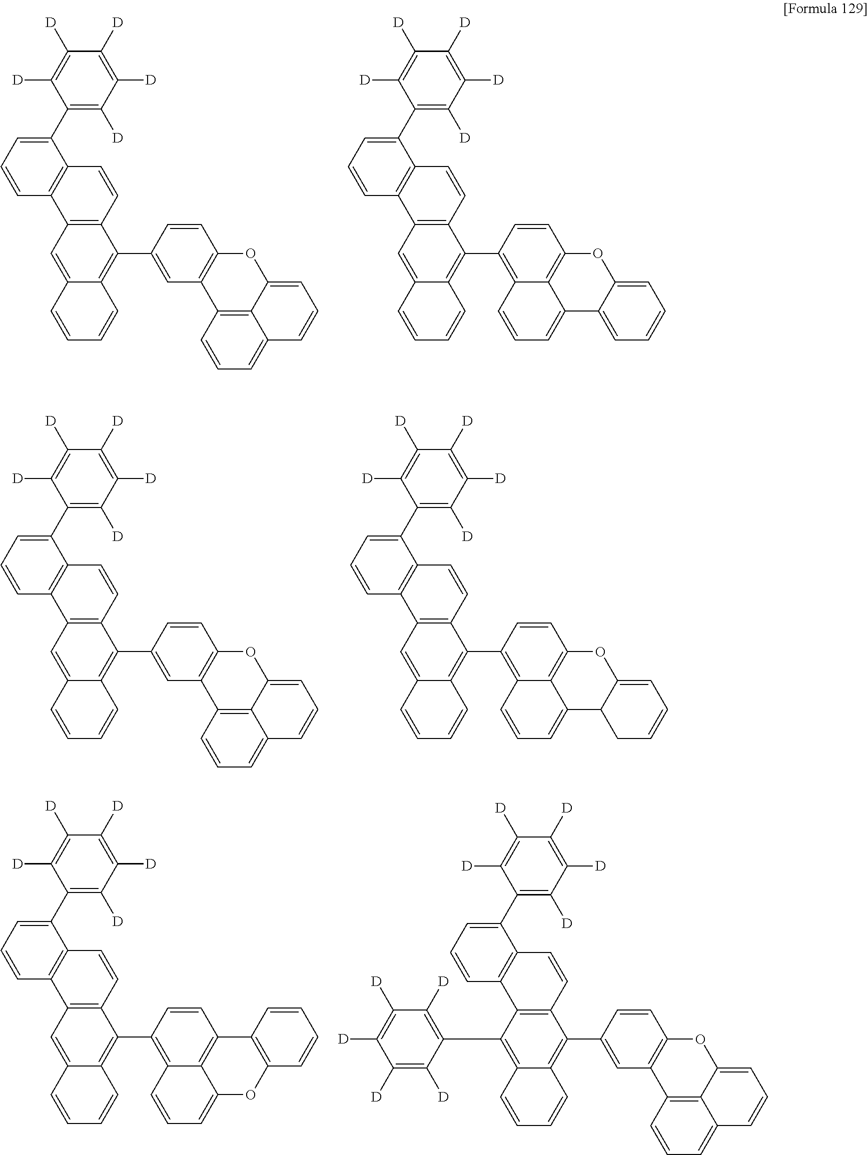

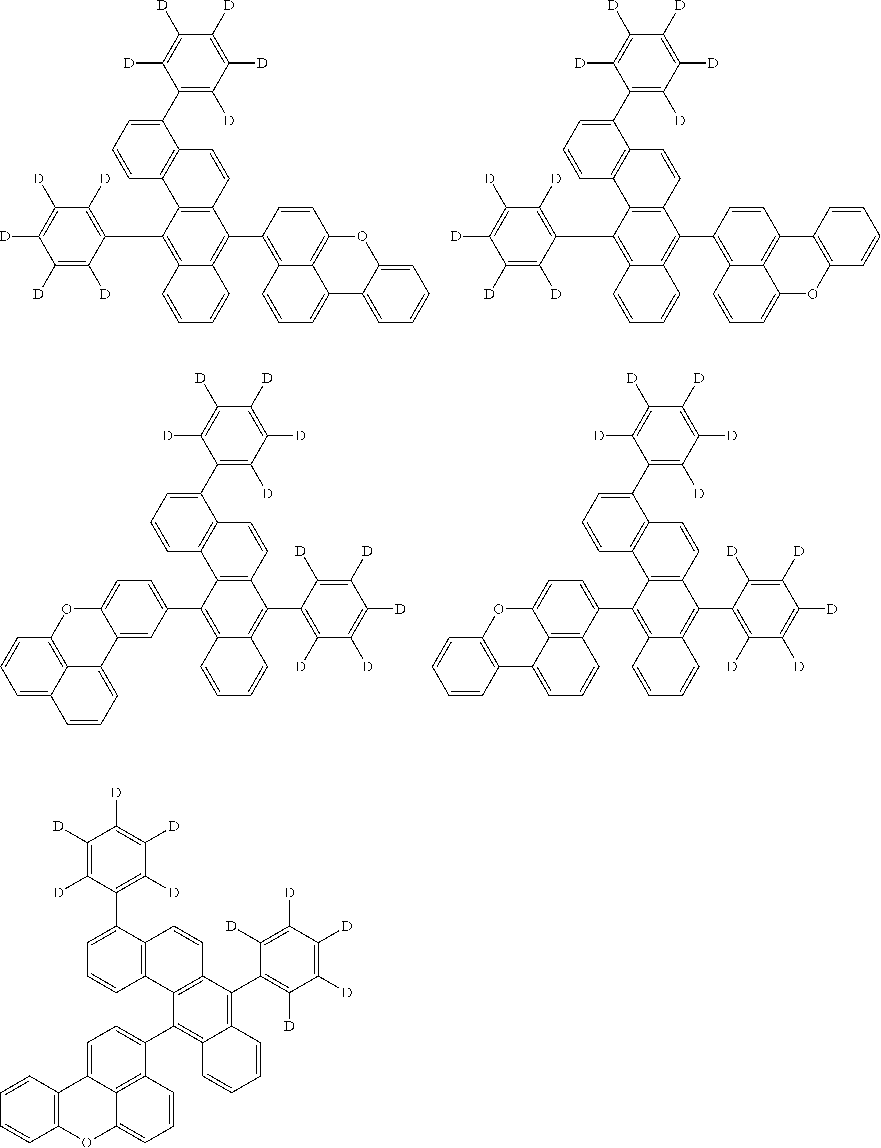

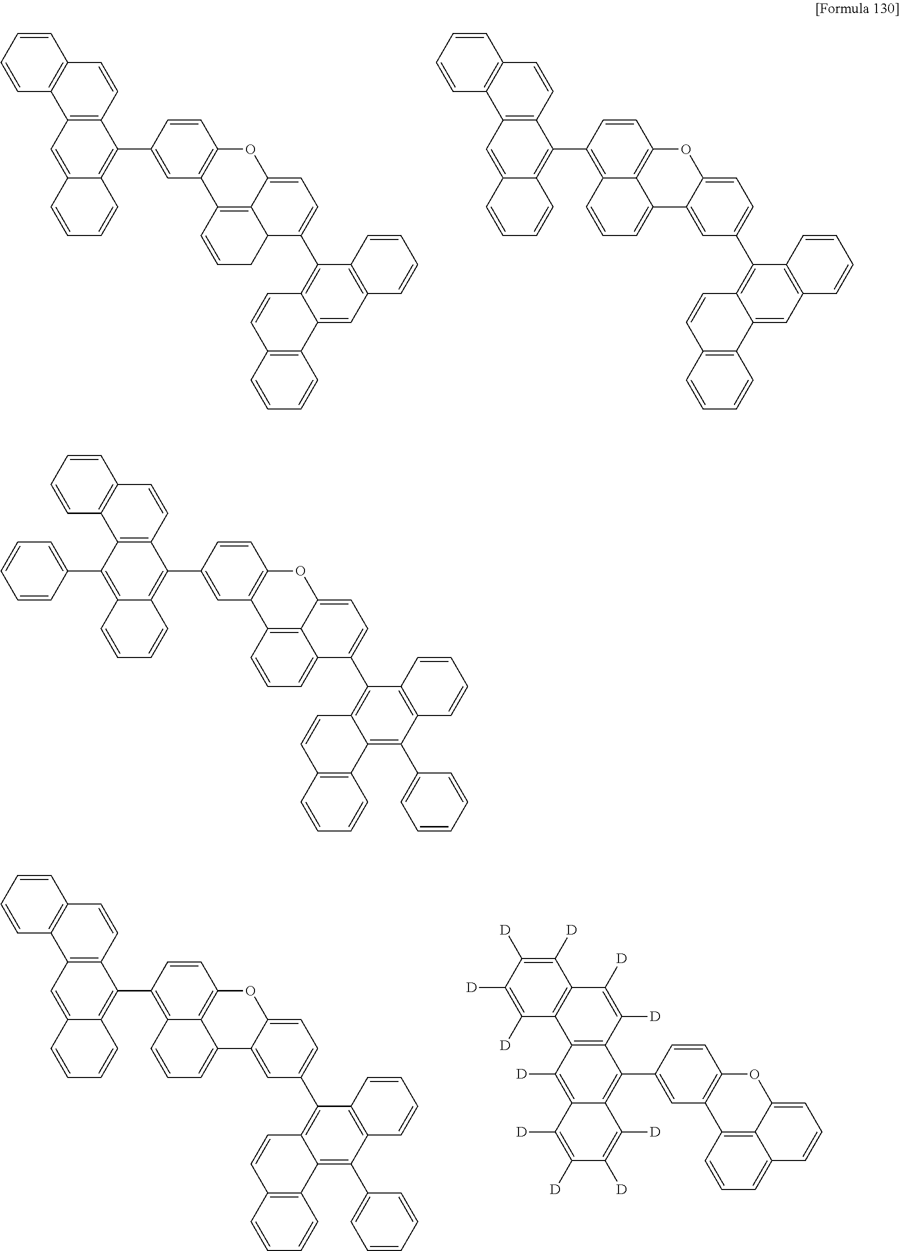

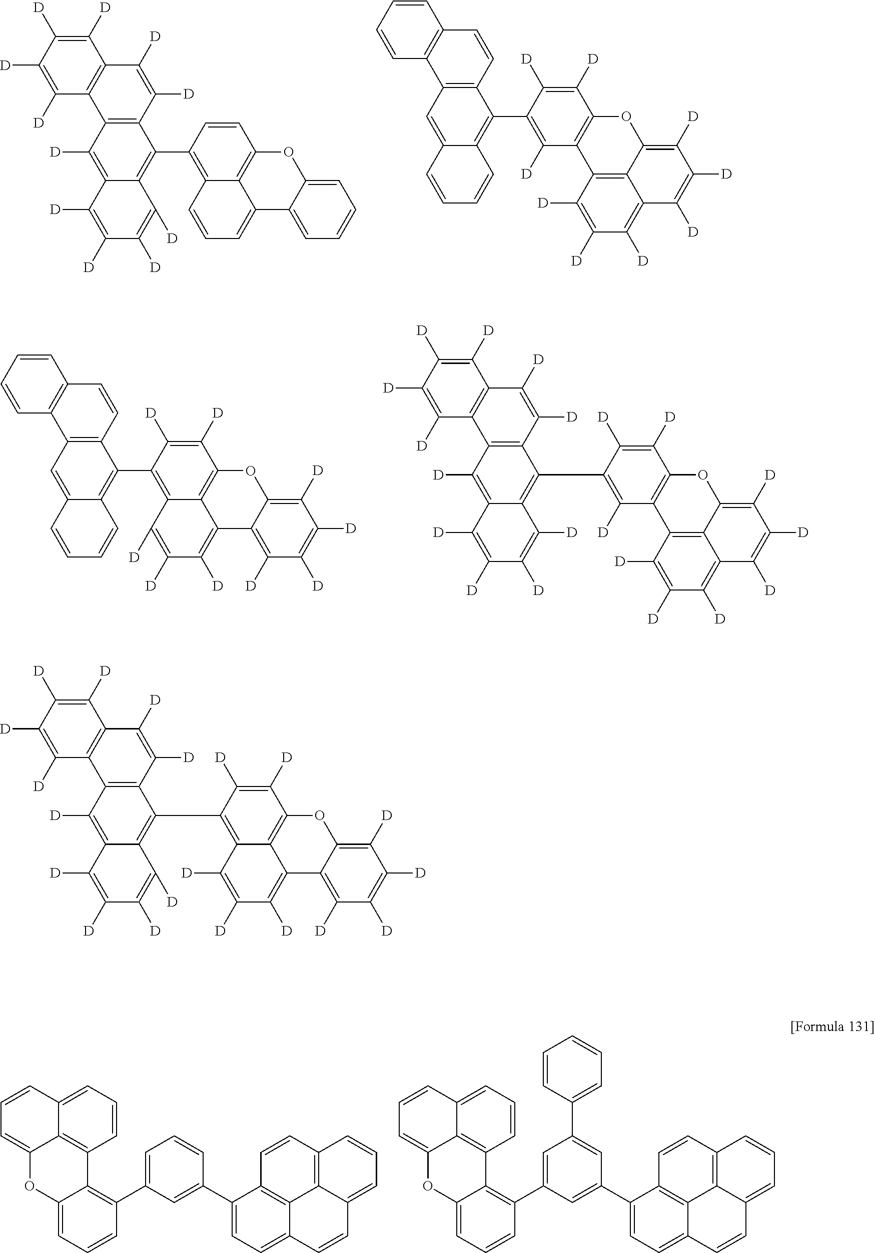

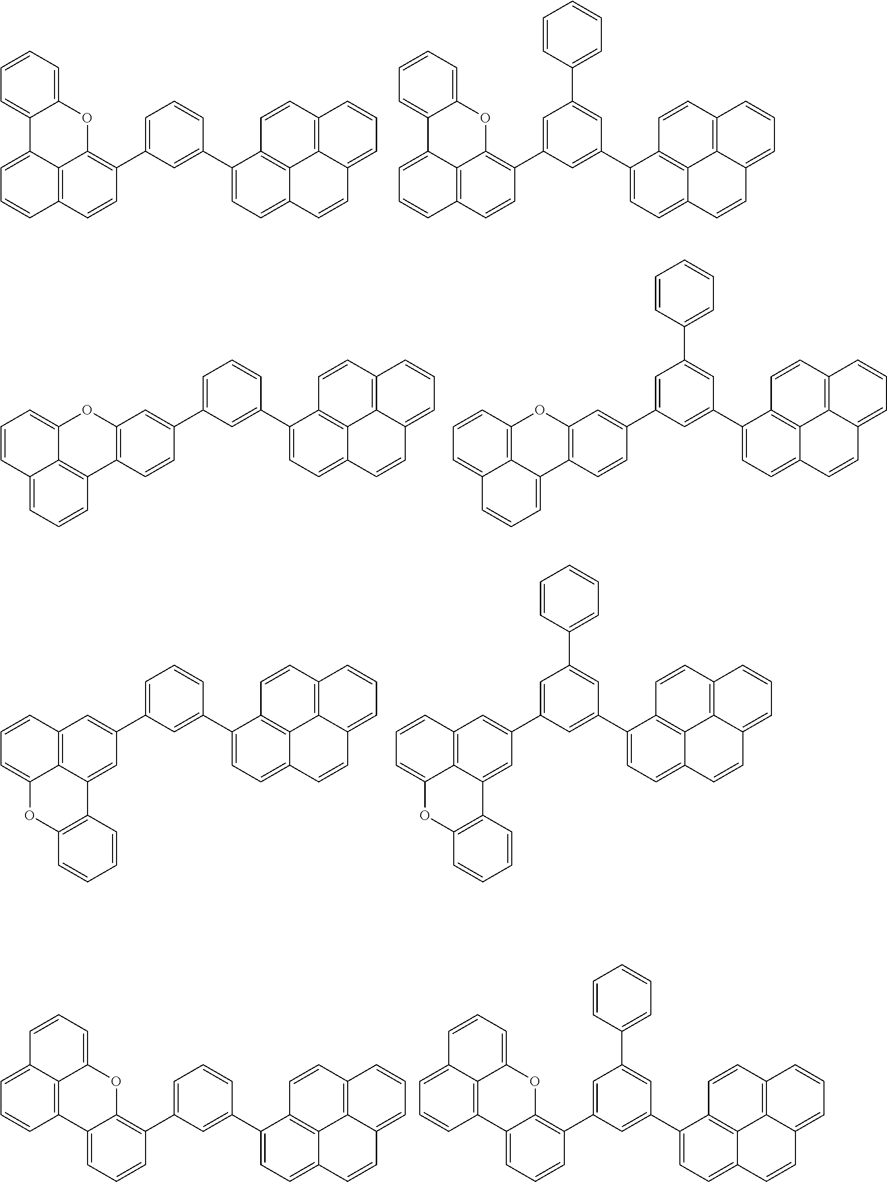

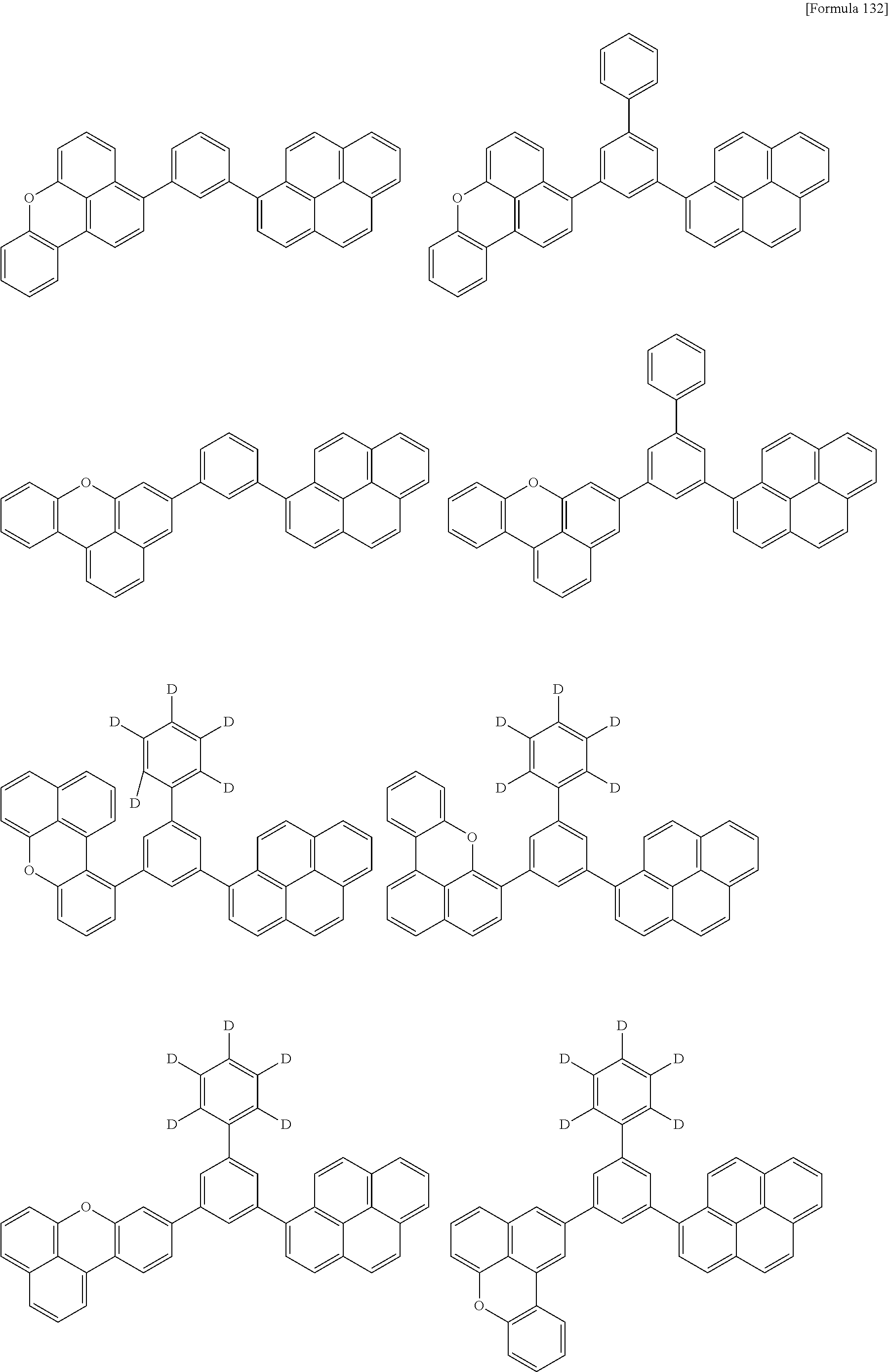

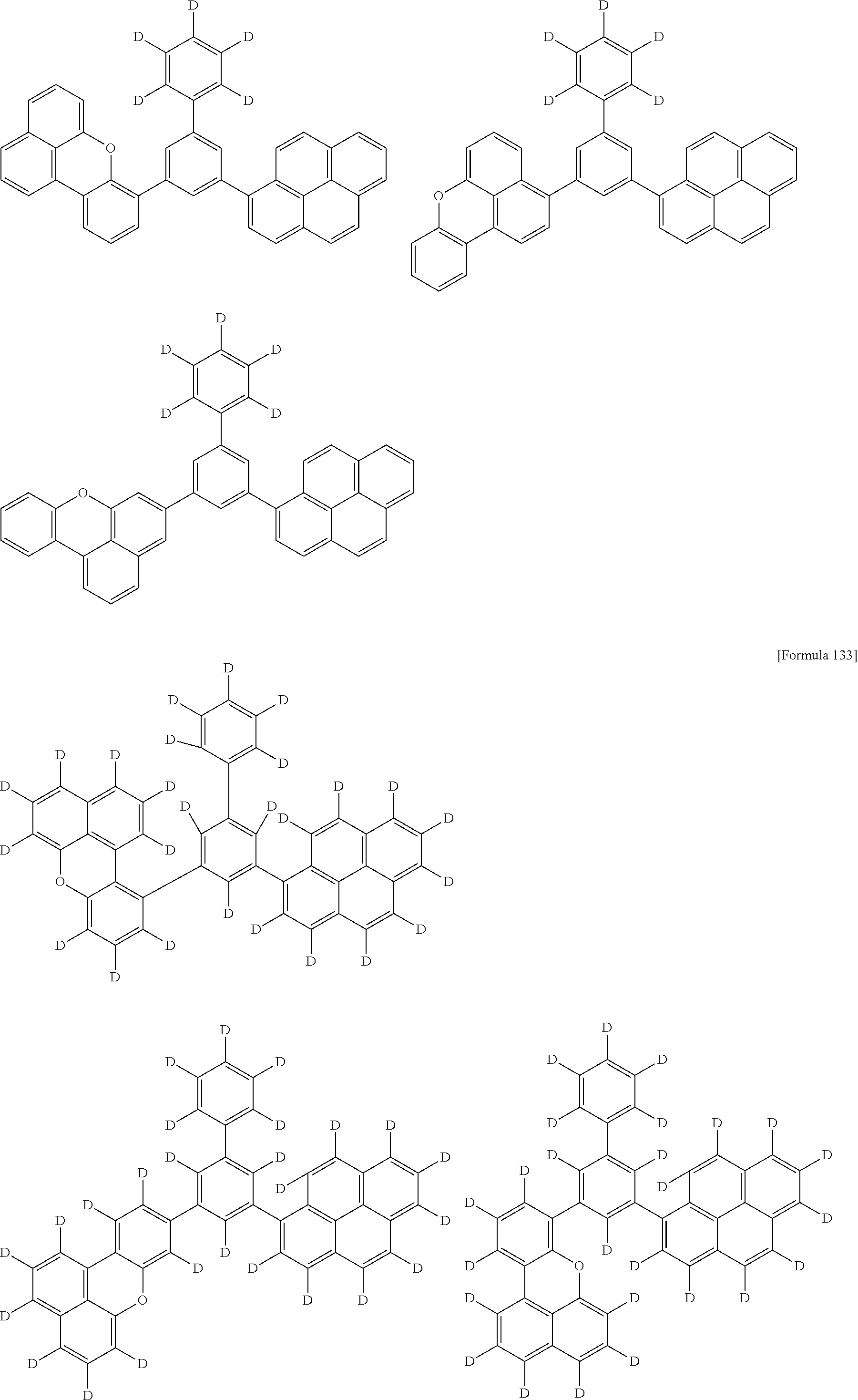

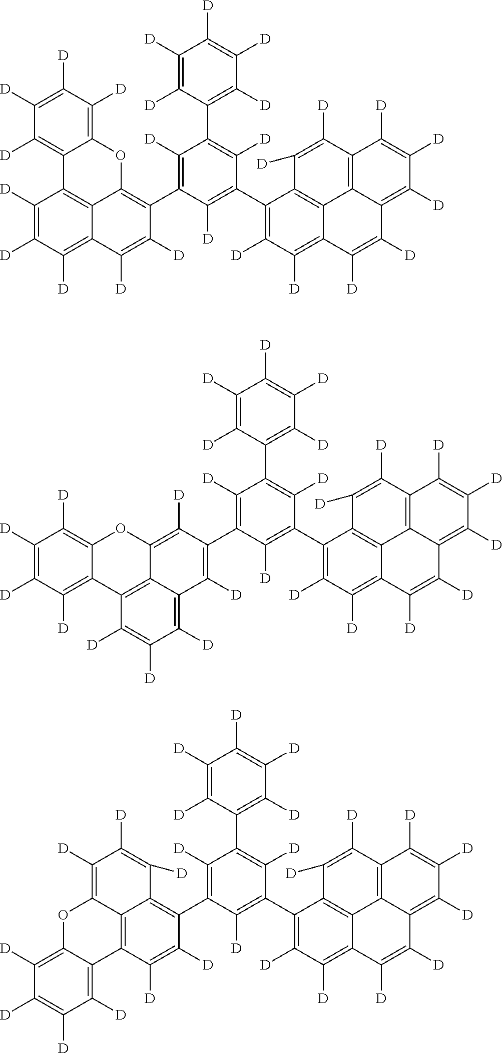

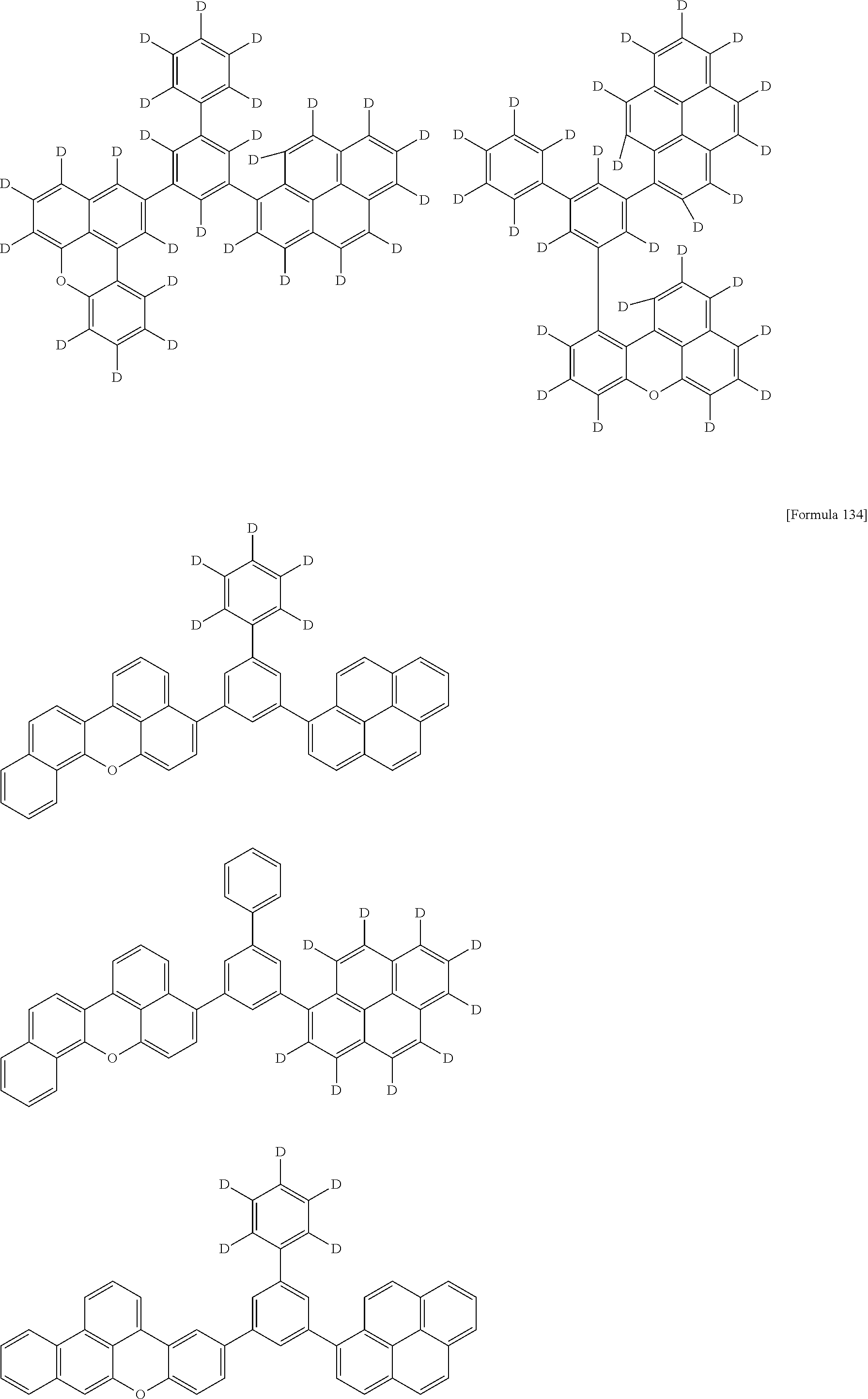

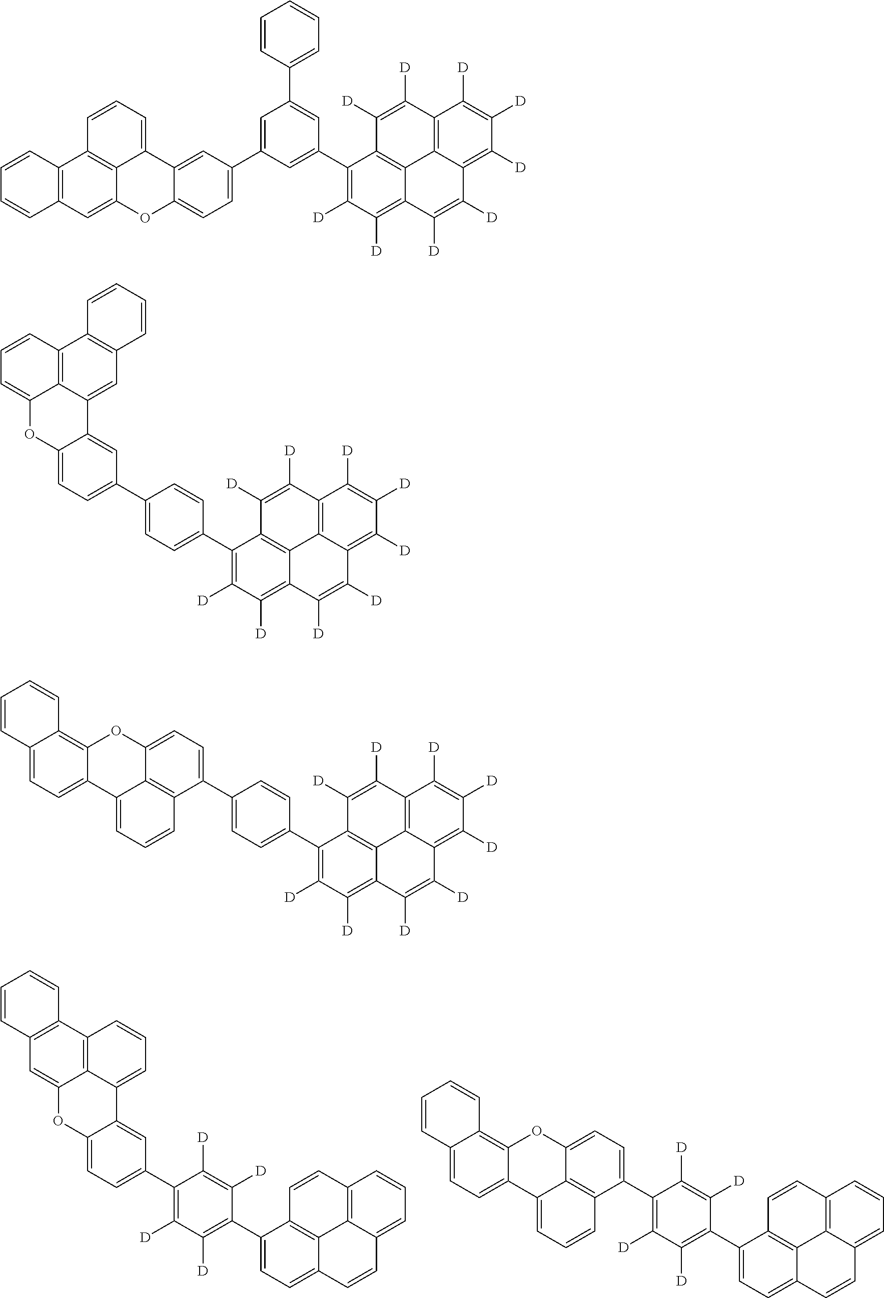

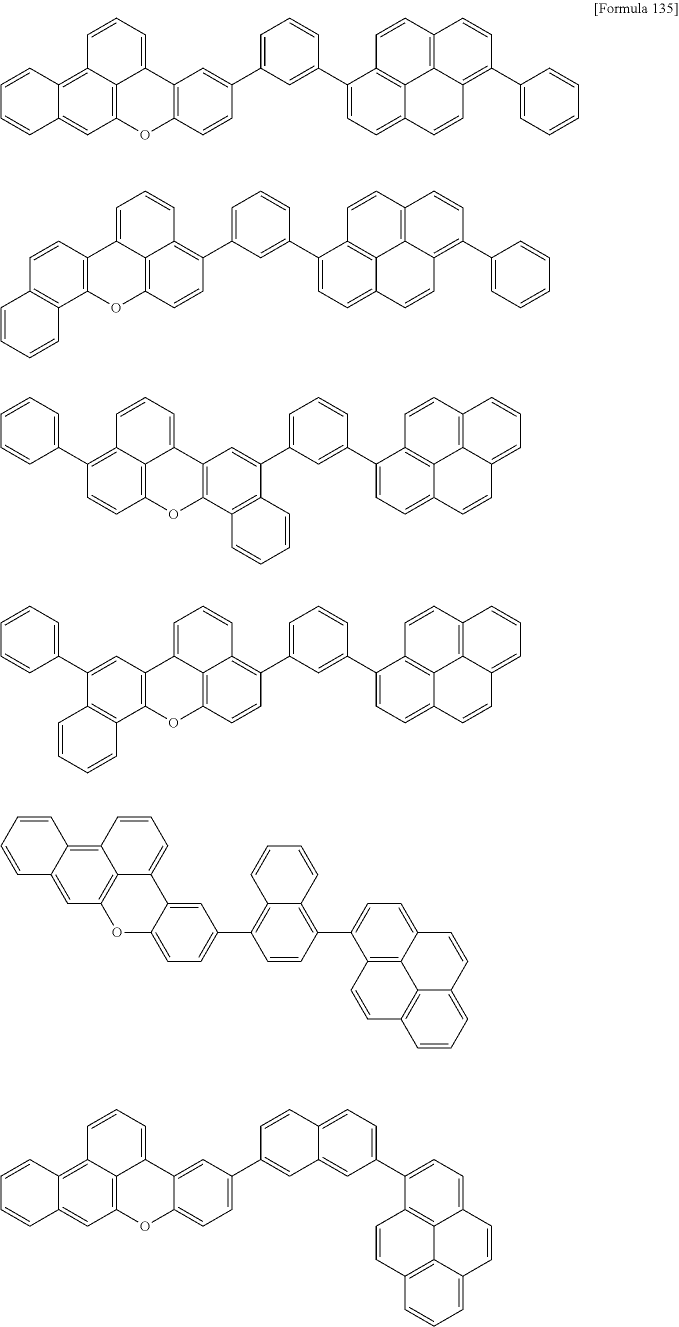

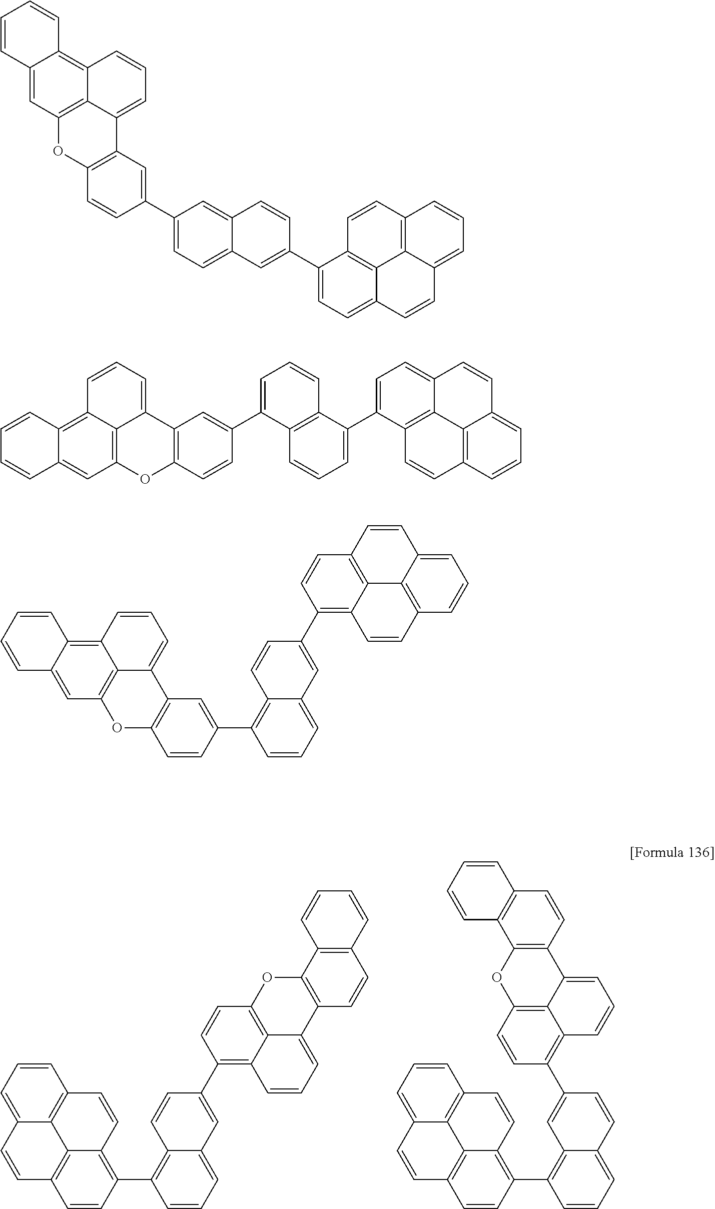

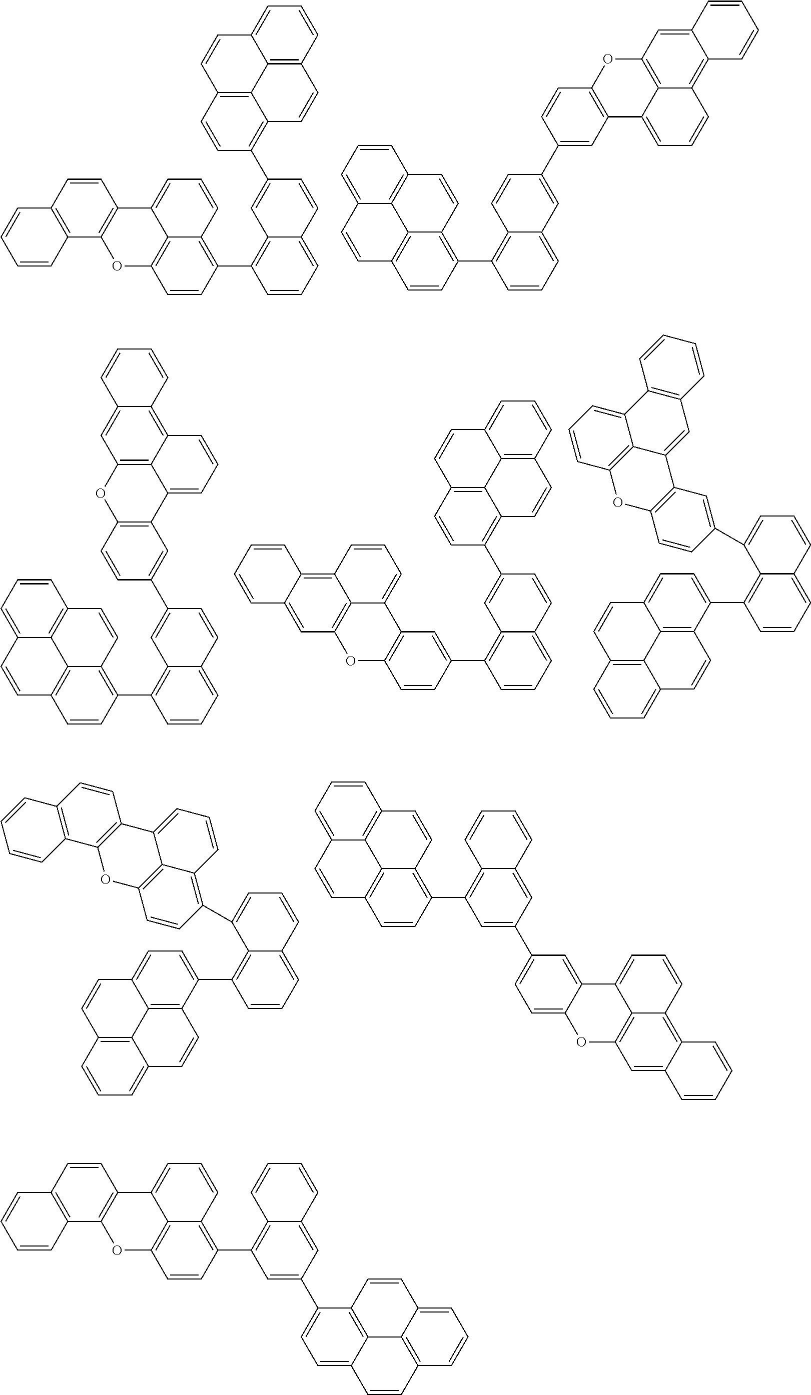

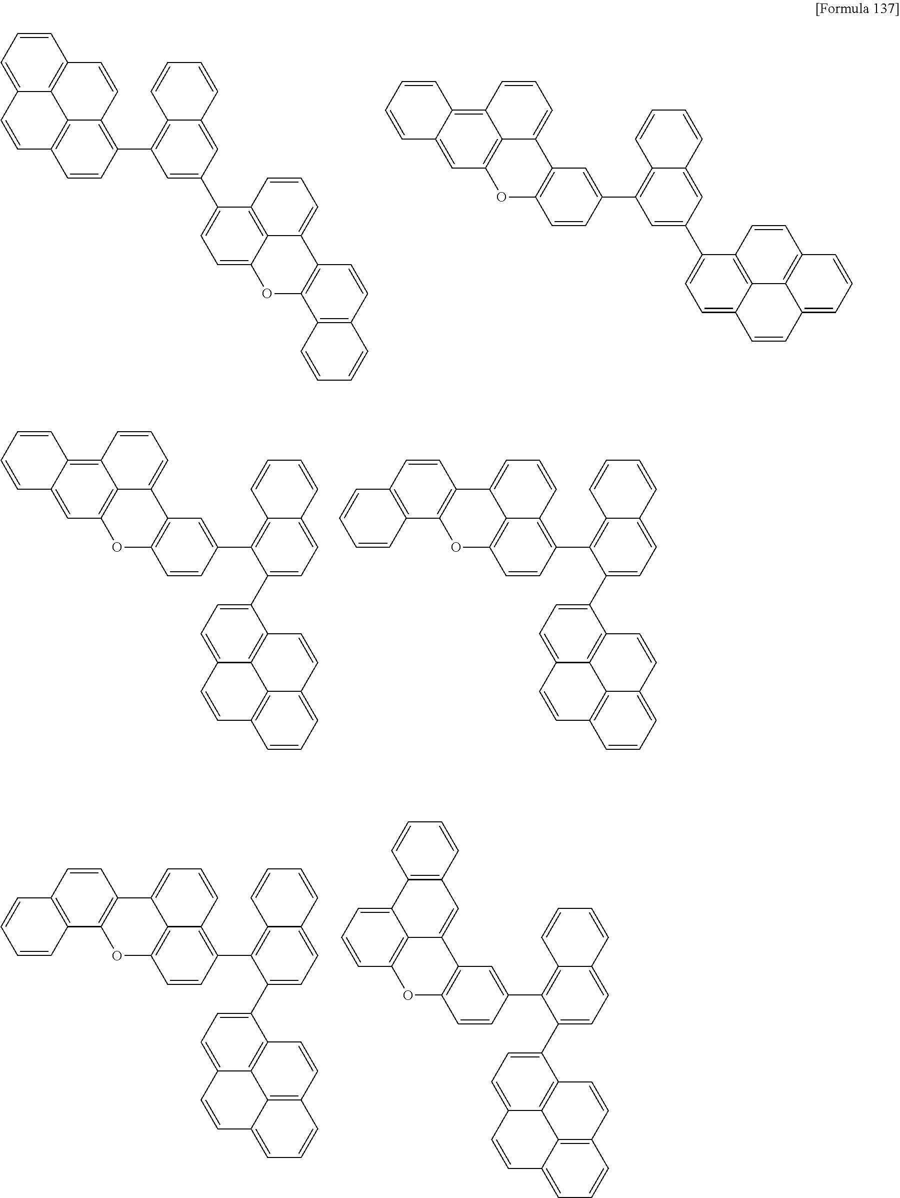

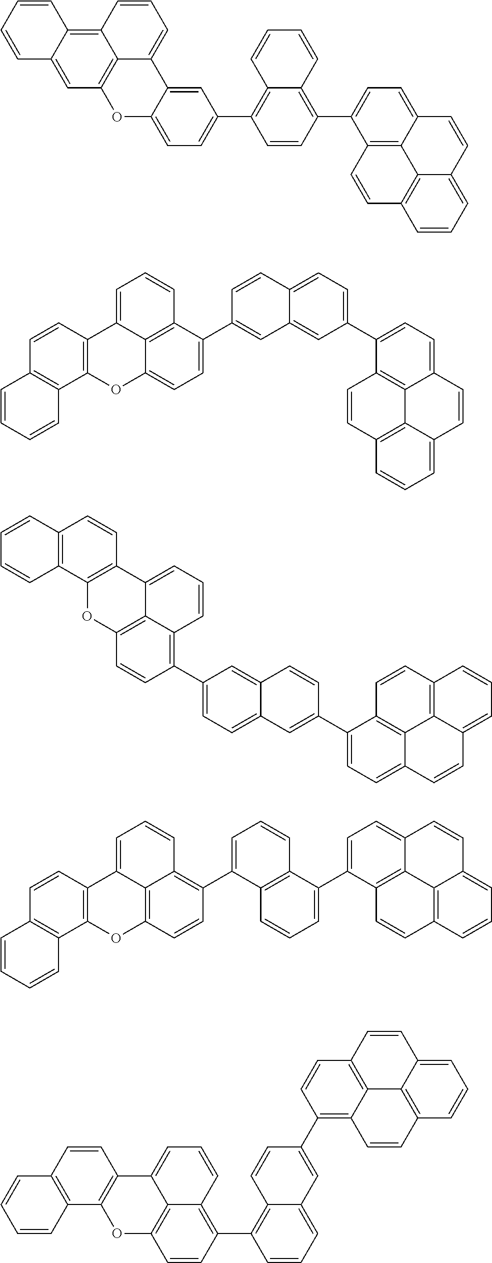

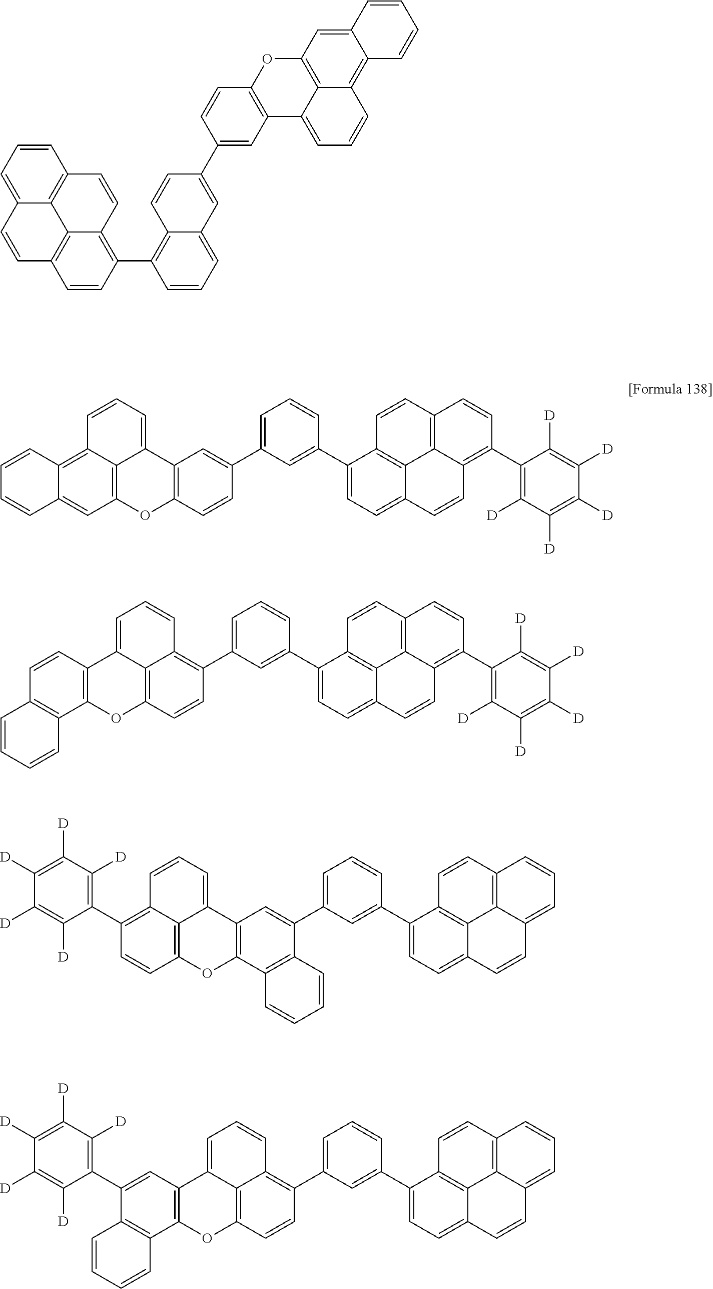

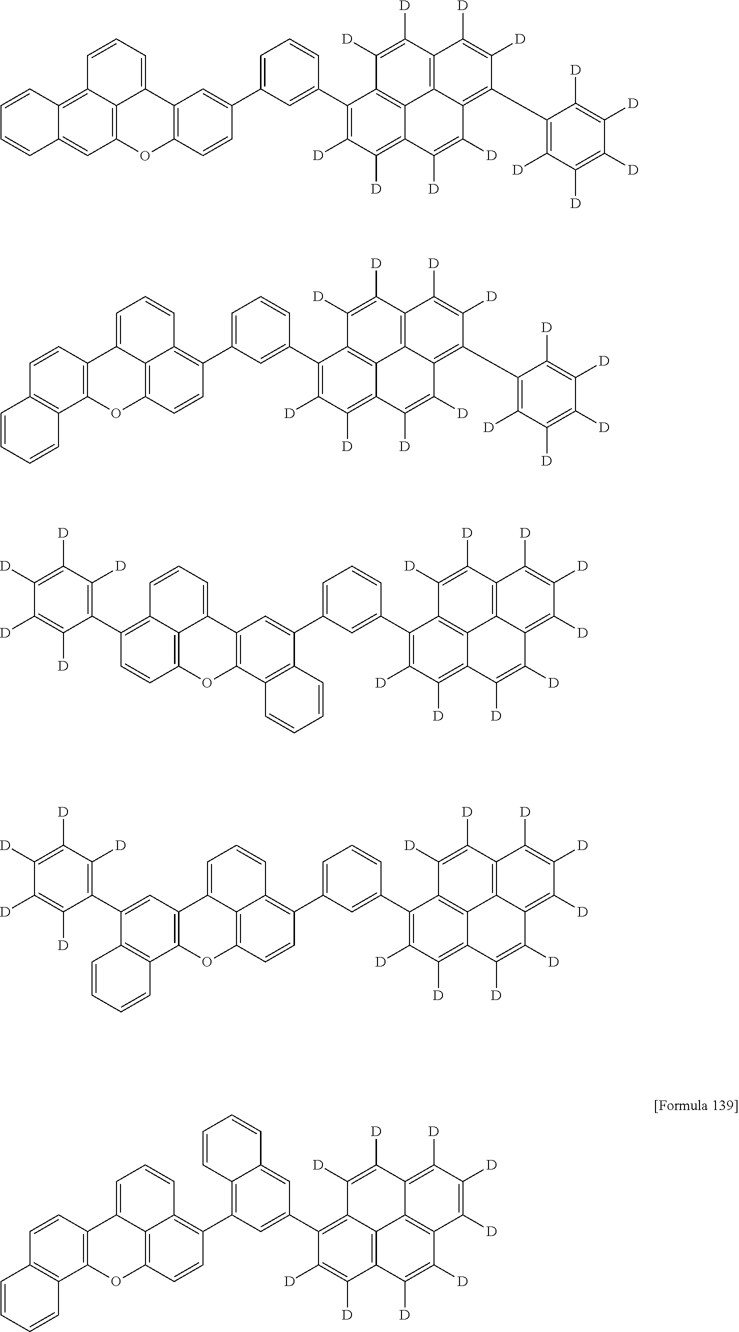

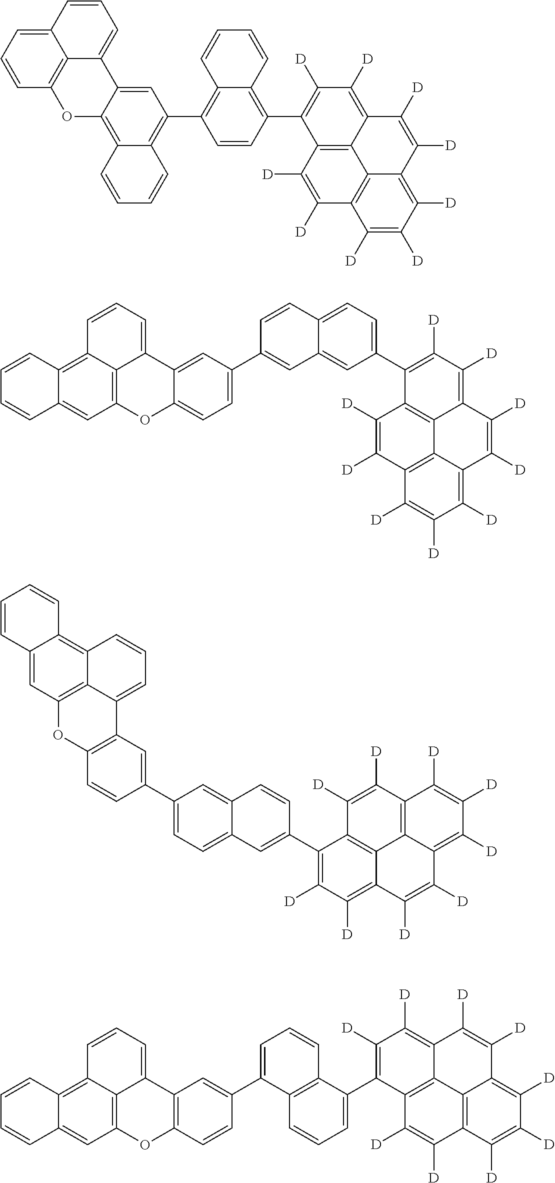

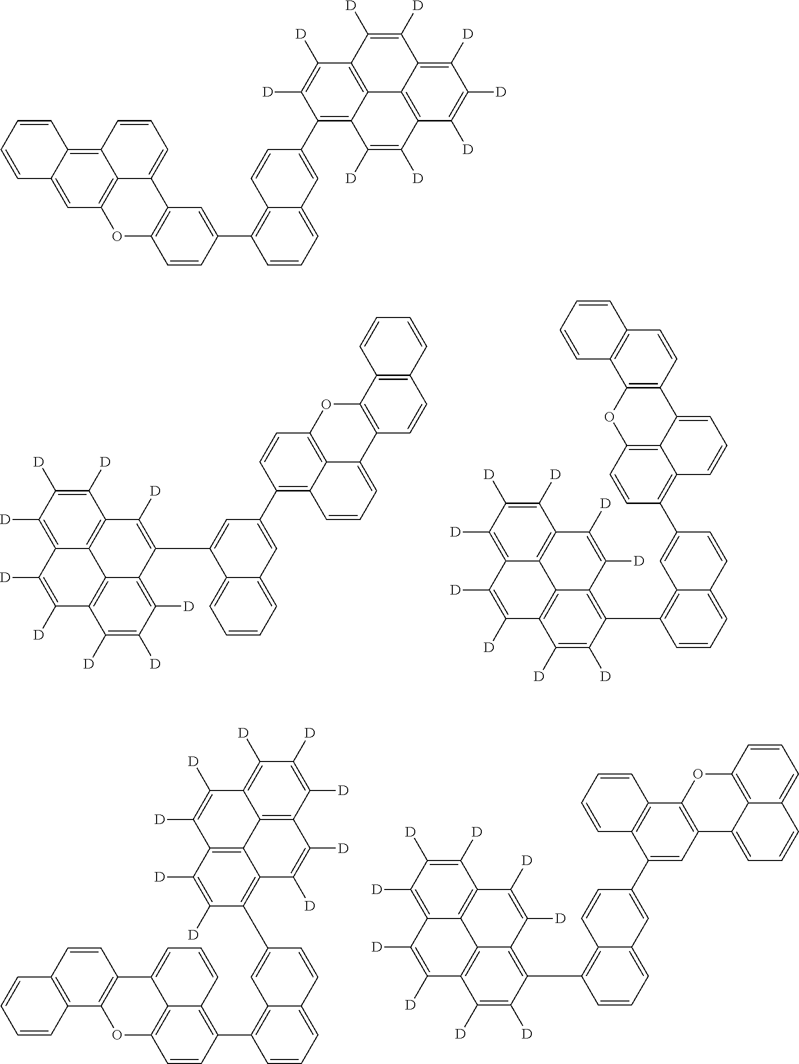

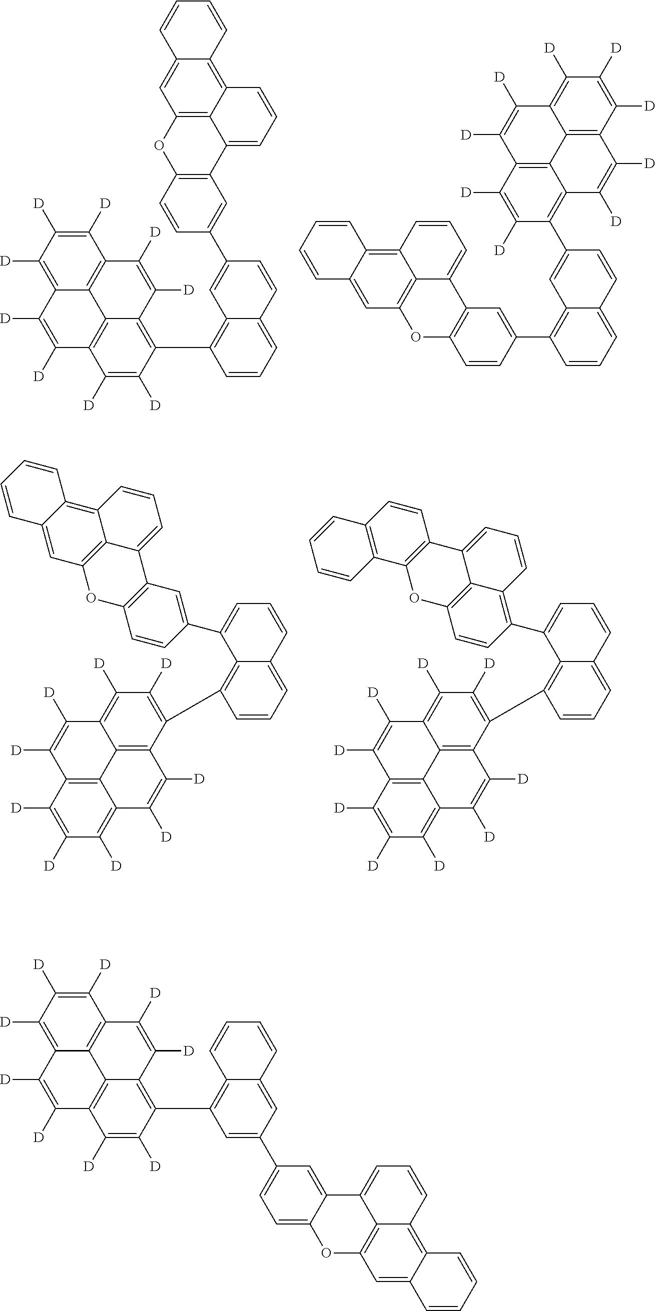

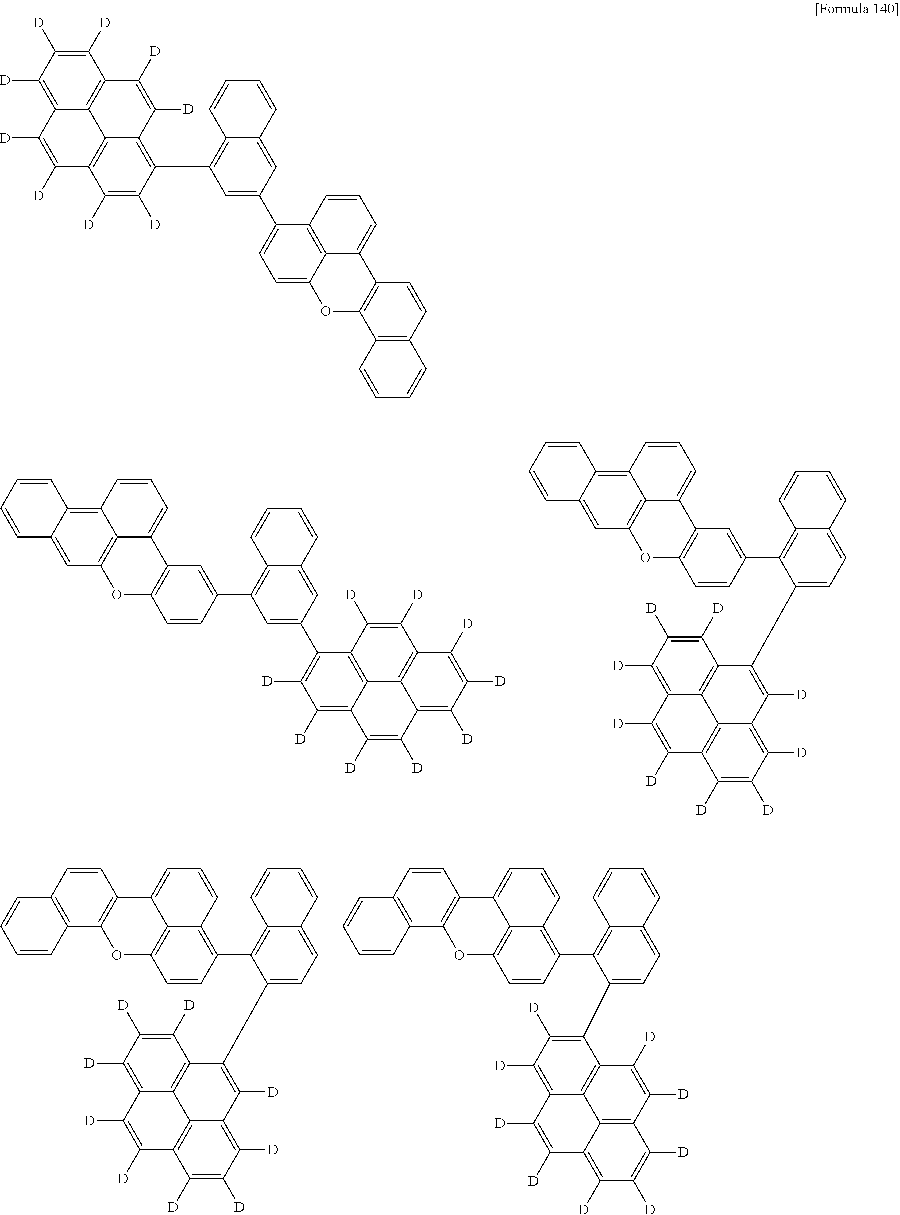

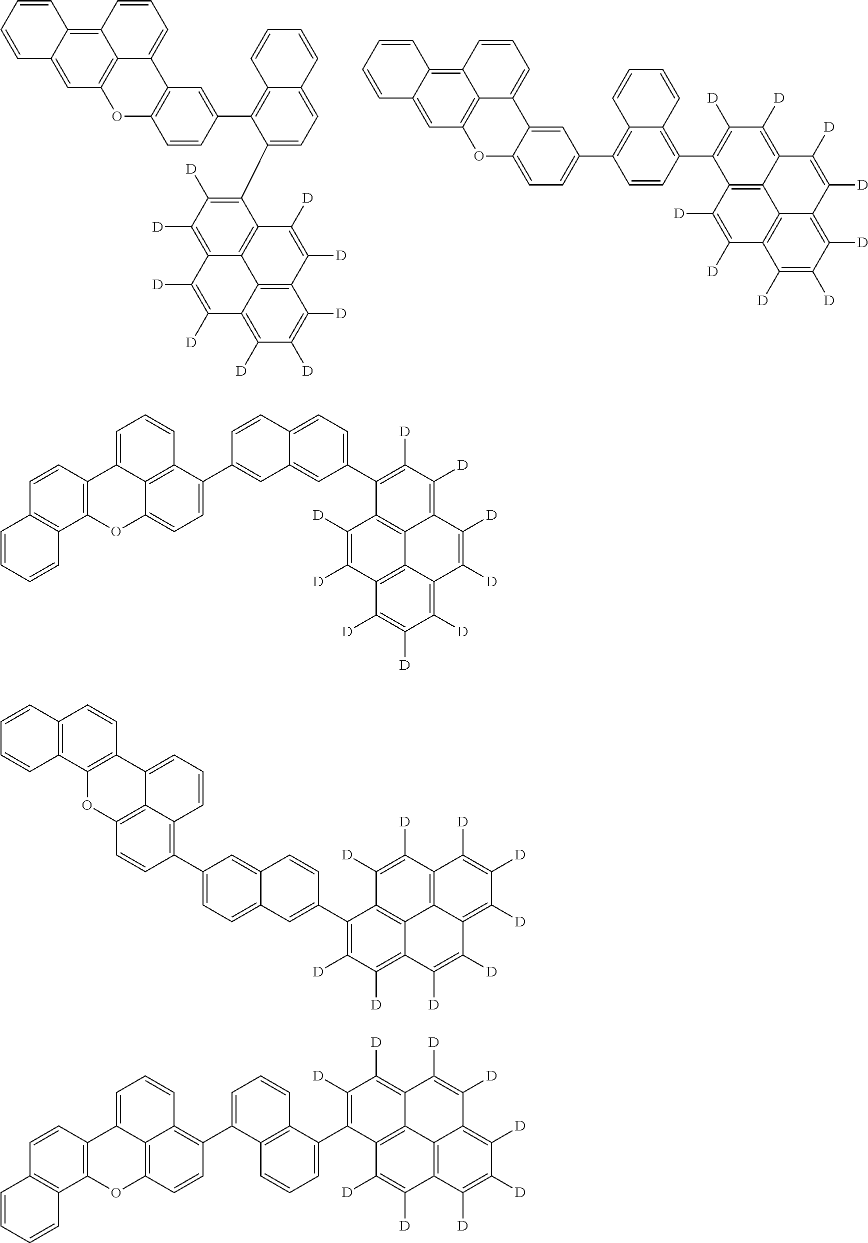

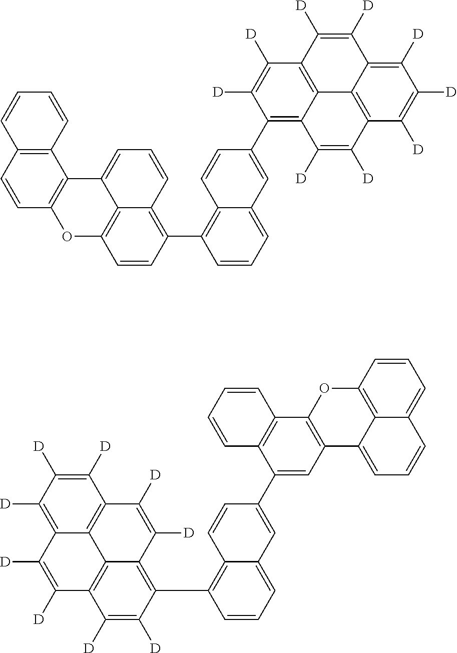 [SMILES: [2H]c1c([2H])c2c([2H])c([2H])c3c([2H])c([2H])c(-c4ccc5c(-c6ccc7c8c(cccc68)-c6c(ccc8ccccc68)O7)cccc5c4)c4c([2H])c([2H])c(c1[2H])c2c34 ; [2H]c1c([2H])c2c([2H])c([2H])c3c([2H])c([2H])c(-c4cccc5cc(-c6cc7c(c8ccccc68)Oc6cccc8cccc-7c68)ccc45)c4c([2H])c([2H])c(c1[2H])c2c34]

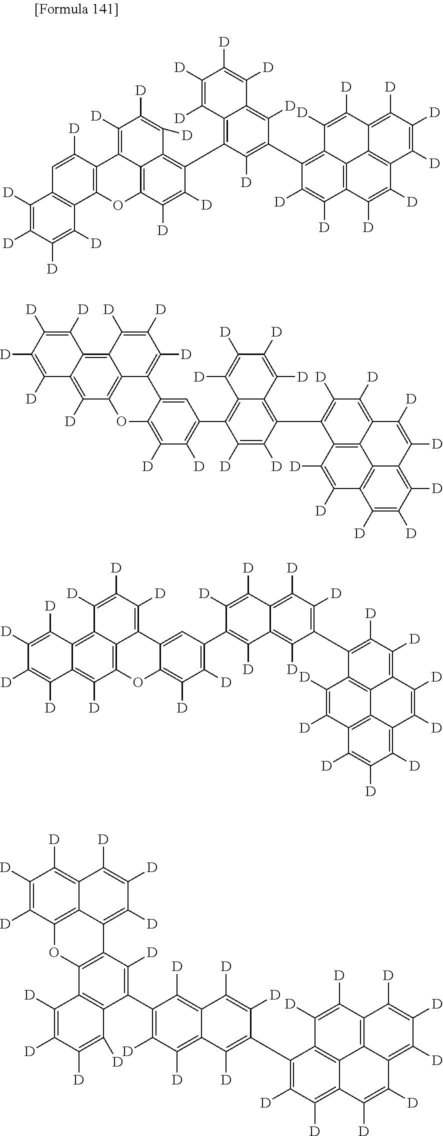

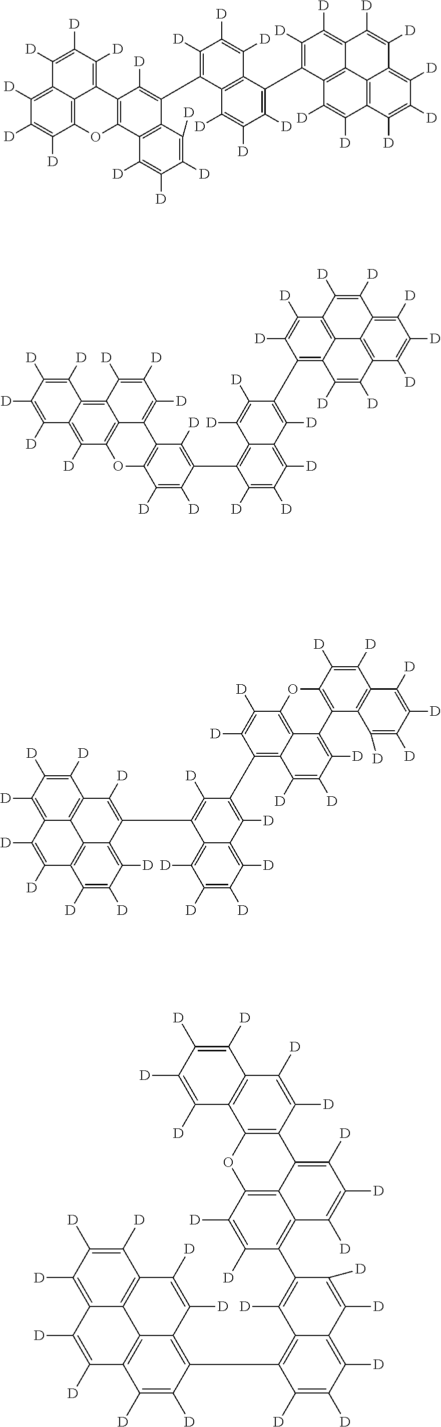

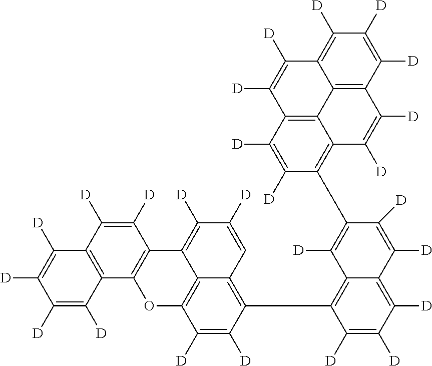 [SMILES: [2H]c1cc2c(-c3c([2H])c([2H])c([2H])c4c([2H])c([2H])c(-c5c([2H])c([2H])c6c([2H])c([2H])c7c([2H])c([2H])c([2H])c8c([2H])c([2H])c5c6c78)c([2H])c34)c([2H])c([2H])c3c2c(c1[2H])-c1c([2H])c([2H])c2c([2H])c([2H])c([2H])c([2H])c2c1O3]

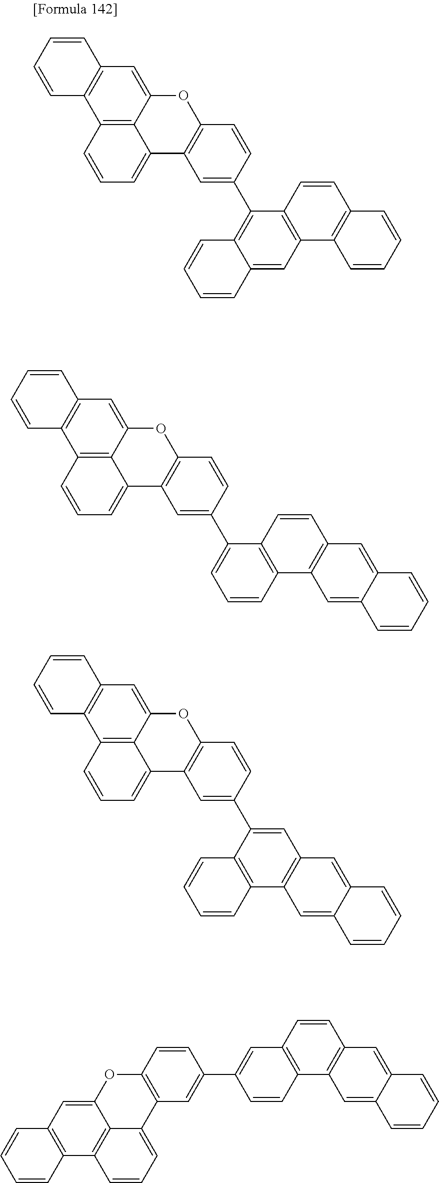 [SMILES: c1ccc2c(-c3ccc4c(c3)-c3cccc5c3c(cc3ccccc35)O4)c3ccc4ccccc4c3cc2c1 ; c1ccc2cc3c(ccc4c(-c5ccc6c(c5)-c5cccc7c5c(cc5ccccc57)O6)cccc43)cc2c1 ; c1ccc2cc3c(cc(-c4ccc5c(c4)-c4cccc6c4c(cc4ccccc46)O5)c4ccccc43)cc2c1 ; c1ccc2cc3c(ccc4cc(-c5ccc6c(c5)-c5cccc7c5c(cc5ccccc57)O6)ccc43)cc2c1]

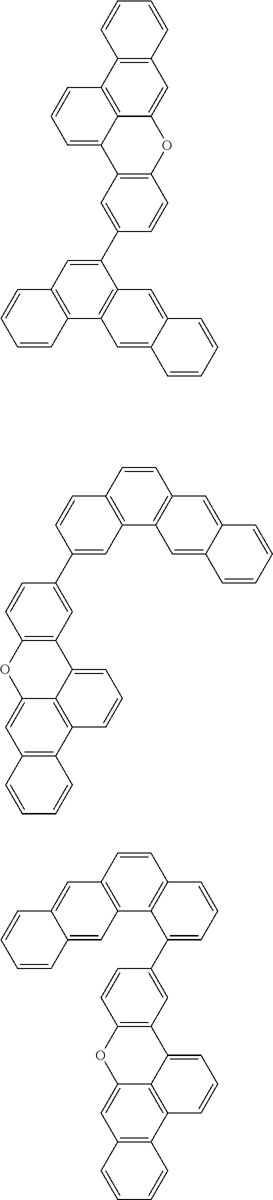 [SMILES: c1ccc2cc3c(cc2c1)c(-c1ccc2c(c1)-c1cccc4c1c(cc1ccccc14)O2)cc1ccccc13 ; c1ccc2cc3c(ccc4ccc(-c5ccc6c(c5)-c5cccc7c5c(cc5ccccc57)O6)cc43)cc2c1 ; c1ccc2cc3c(ccc4cccc(-c5ccc6c(c5)-c5cccc7c5c(cc5ccccc57)O6)c43)cc2c1]

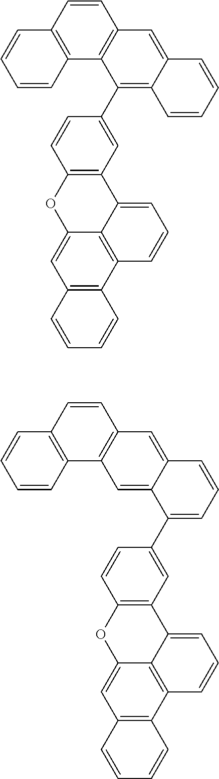 [SMILES: c1ccc2c(-c3ccc4c(c3)-c3cccc5c3c(cc3ccccc35)O4)c3c(ccc4ccccc43)cc2c1 ; c1cc(-c2ccc3c(c2)-c2cccc4c2c(cc2ccccc24)O3)c2cc3c(ccc4ccccc43)cc2c1]

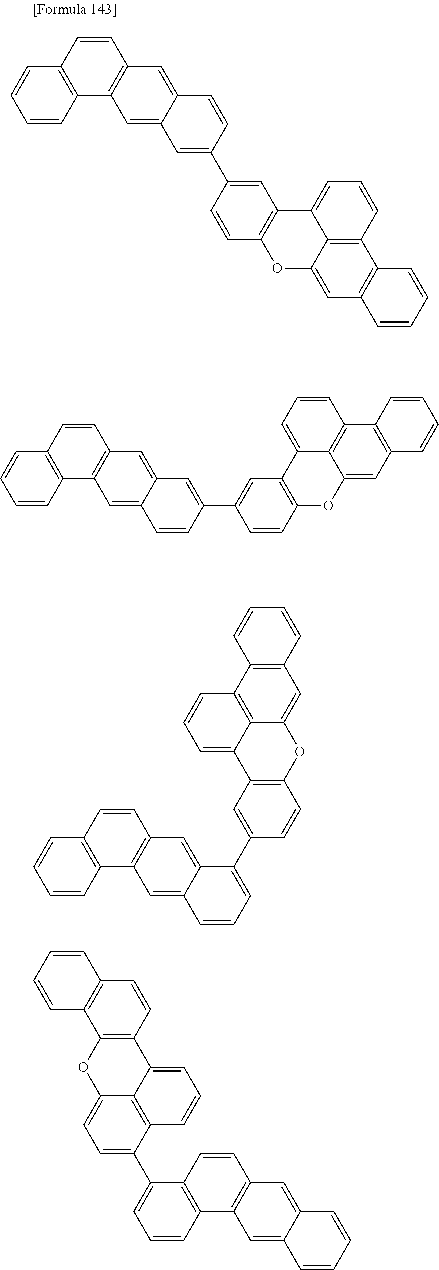 [SMILES: c1ccc2c(c1)ccc1cc3ccc(-c4ccc5c(c4)-c4cccc6c4c(cc4ccccc46)O5)cc3cc12 ; c1ccc2c(c1)ccc1cc3cc(-c4ccc5c(c4)-c4cccc6c4c(cc4ccccc46)O5)ccc3cc12 ; c1cc(-c2ccc3c(c2)-c2cccc4c2c(cc2ccccc24)O3)c2cc3ccc4ccccc4c3cc2c1 ; c1ccc2cc3c(ccc4c(-c5ccc6c7c(cccc57)-c5ccc7ccccc7c5O6)cccc43)cc2c1]

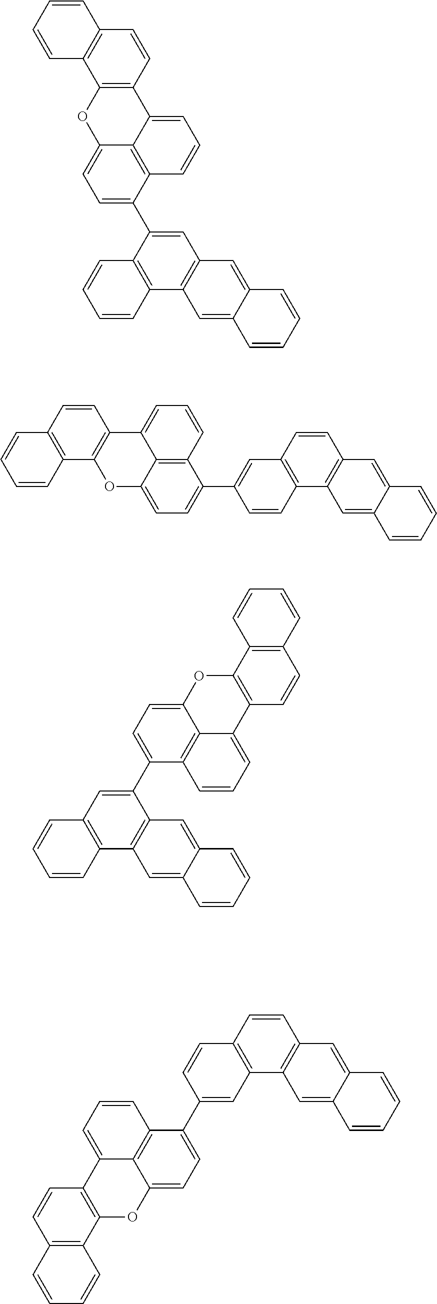 [SMILES: c1ccc2cc3c(cc(-c4ccc5c6c(cccc46)-c4ccc6ccccc6c4O5)c4ccccc43)cc2c1 ; c1ccc2cc3c(ccc4cc(-c5ccc6c7c(cccc57)-c5ccc7ccccc7c5O6)ccc43)cc2c1 ; c1ccc2cc3c(cc2c1)c(-c1ccc2c4c(cccc14)-c1ccc4ccccc4c1O2)cc1ccccc13 ; c1ccc2cc3c(ccc4ccc(-c5ccc6c7c(cccc57)-c5ccc7ccccc7c5O6)cc43)cc2c1]

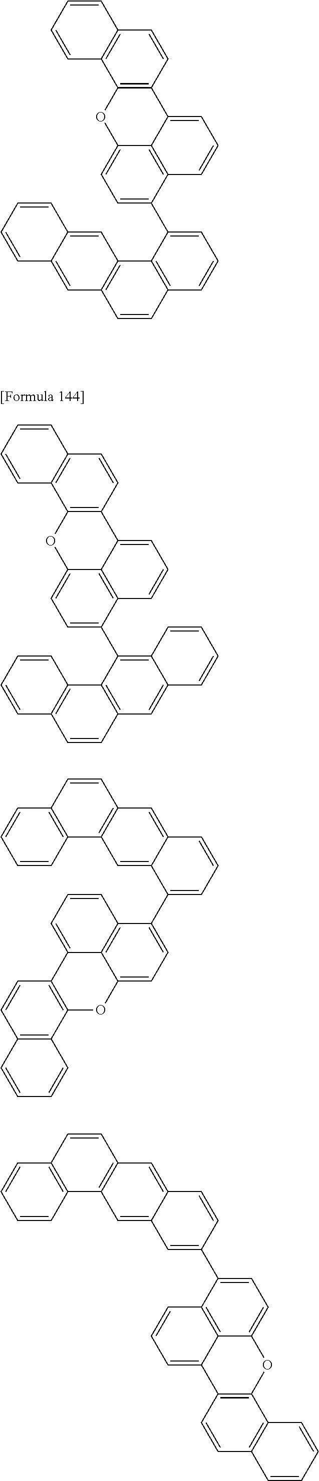

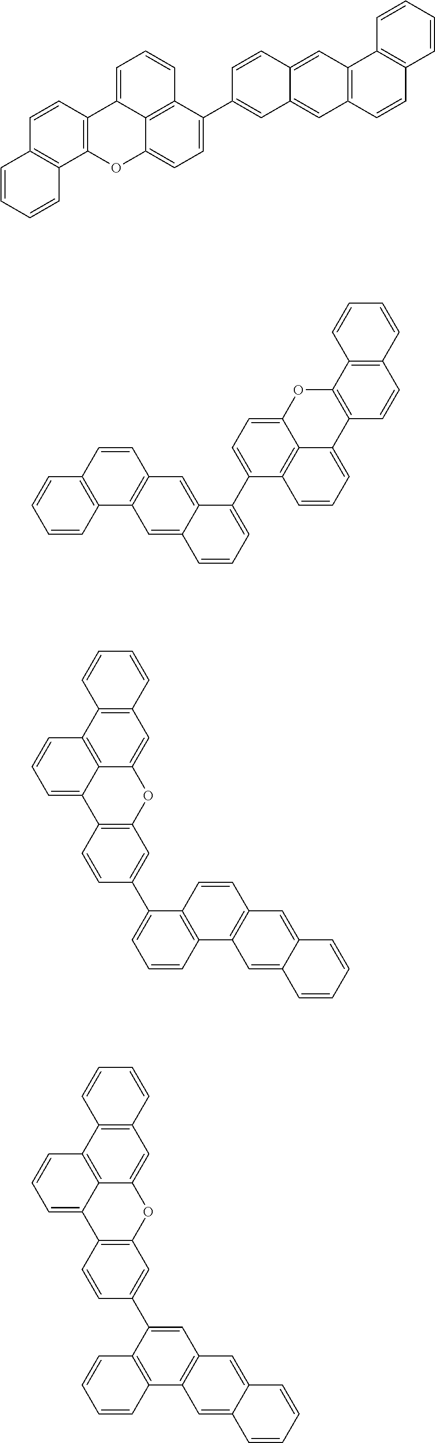 [SMILES: c1ccc2c3c(ccc2c1)-c1cccc2c(-c4ccc5cc6c(ccc7ccccc76)cc5c4)ccc(c12)O3 ; c1cc(-c2ccc3c4c(cccc24)-c2ccc4ccccc4c2O3)c2cc3ccc4ccccc4c3cc2c1 ; c1ccc2cc3c(ccc4c(-c5ccc6c(c5)Oc5cc7ccccc7c7cccc-6c57)cccc43)cc2c1 ; c1ccc2cc3c(cc(-c4ccc5c(c4)Oc4cc6ccccc6c6cccc-5c46)c4ccccc43)cc2c1]

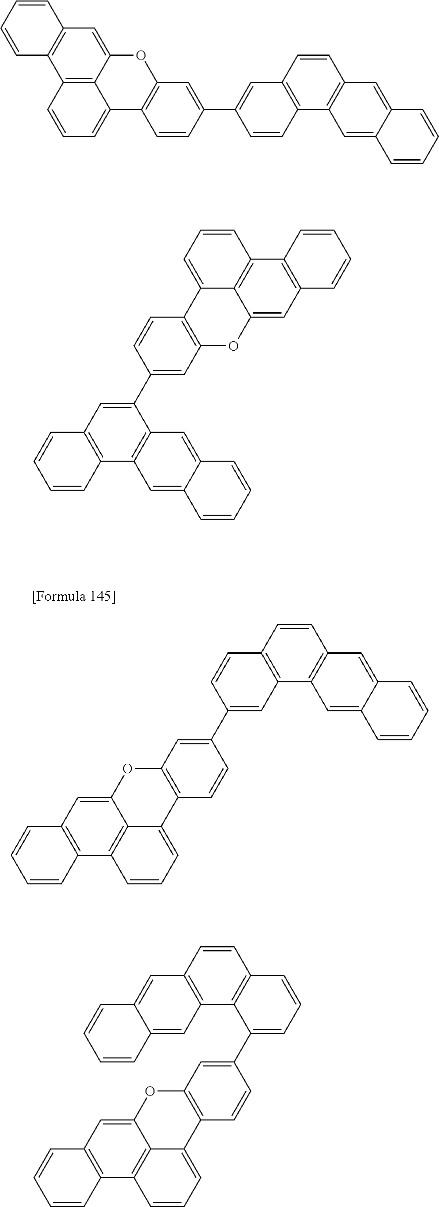 [SMILES: c1ccc2cc3c(ccc4cc(-c5ccc6c(c5)Oc5cc7ccccc7c7cccc-6c57)ccc43)cc2c1 ; c1ccc2cc3c(cc2c1)c(-c1ccc2c(c1)Oc1cc4ccccc4c4cccc-2c14)cc1ccccc13 ; c1ccc2cc3c(ccc4ccc(-c5ccc6c(c5)Oc5cc7ccccc7c7cccc-6c57)cc43)cc2c1 ; c1ccc2cc3c(ccc4cccc(-c5ccc6c(c5)Oc5cc7ccccc7c7cccc-6c57)c43)cc2c1]

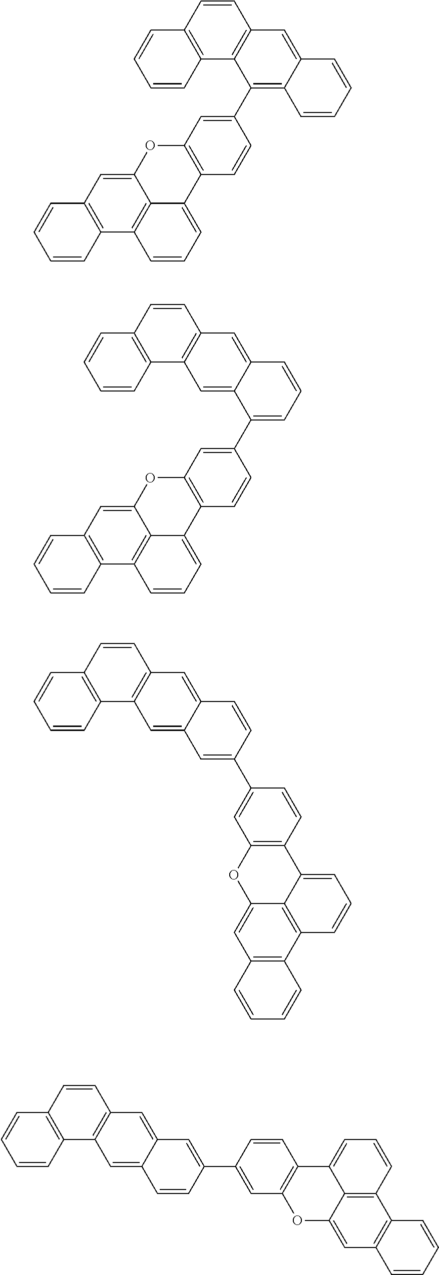

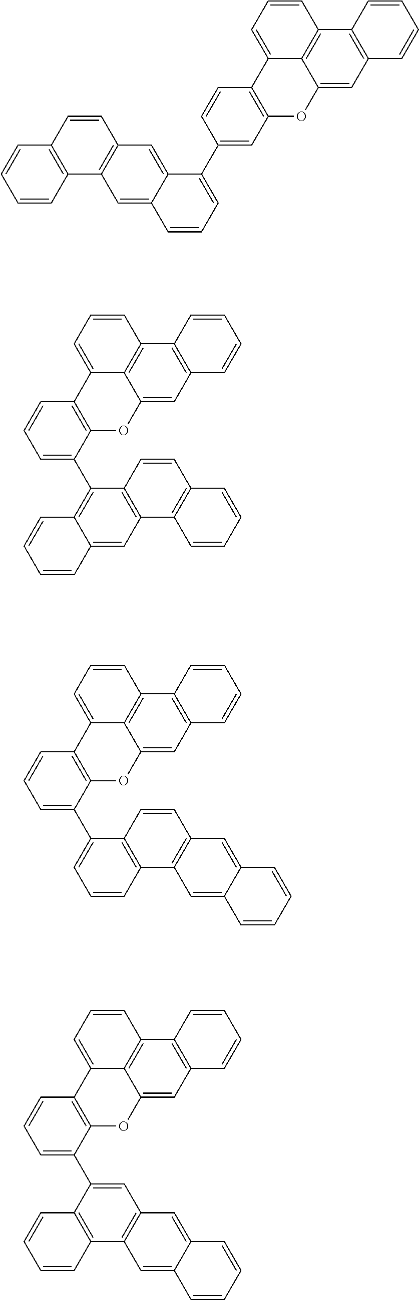 [SMILES: c1cc(-c2ccc3c(c2)Oc2cc4ccccc4c4cccc-3c24)c2cc3ccc4ccccc4c3cc2c1 ; c1cc(-c2c3ccccc3cc3c2ccc2ccccc23)c2c(c1)-c1cccc3c1c(cc1ccccc13)O2 ; c1cc(-c2cccc3c2ccc2cc4ccccc4cc23)c2c(c1)-c1cccc3c1c(cc1ccccc13)O2 ; c1cc(-c2cc3cc4ccccc4cc3c3ccccc23)c2c(c1)-c1cccc3c1c(cc1ccccc13)O2]

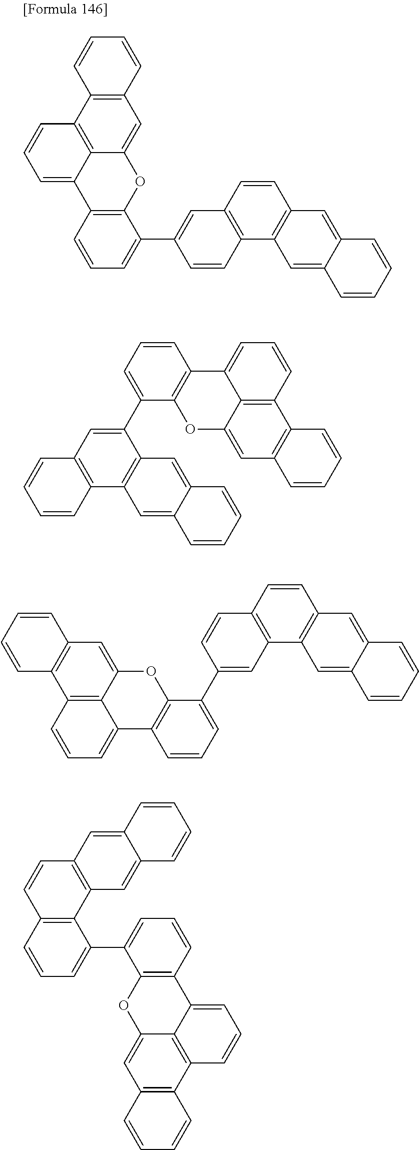

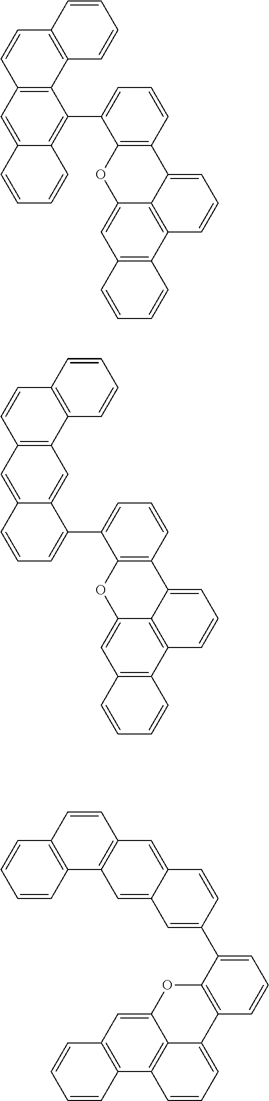

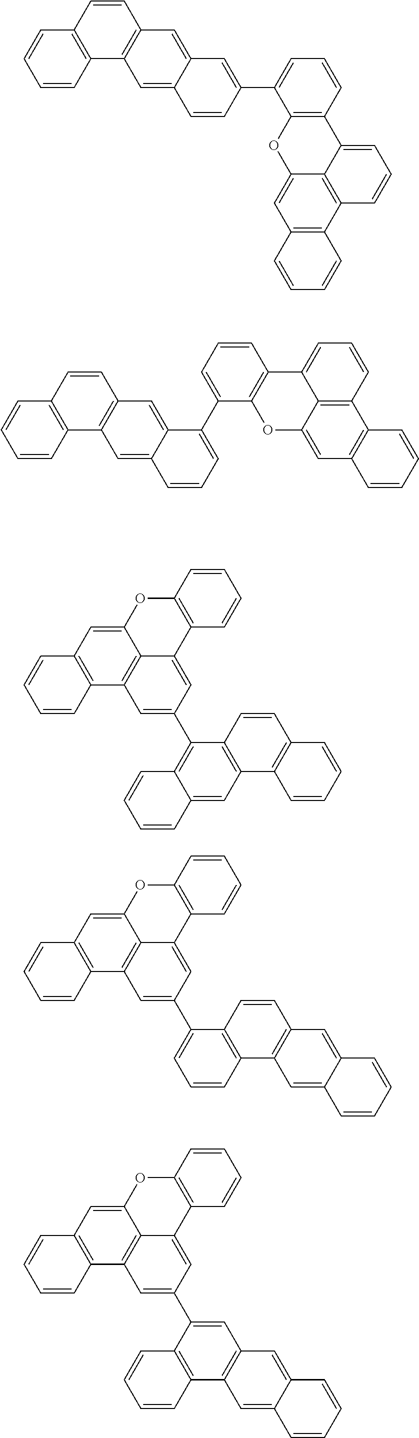

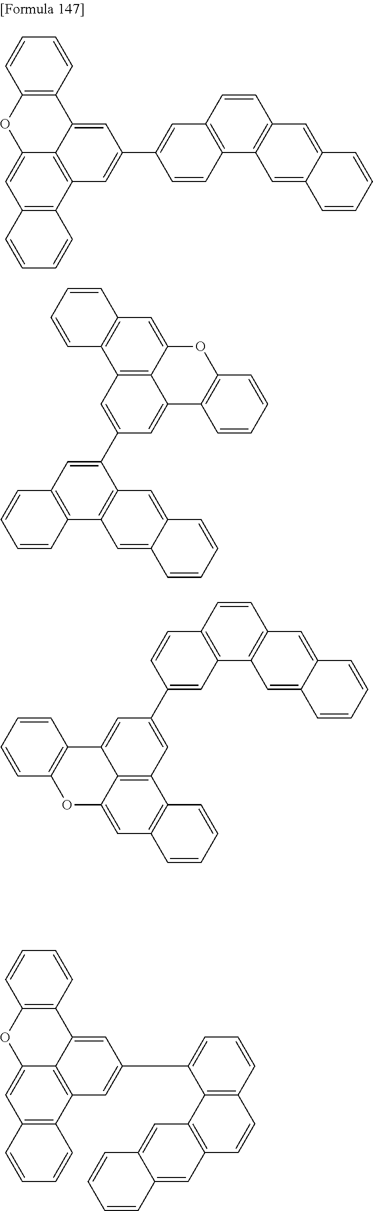

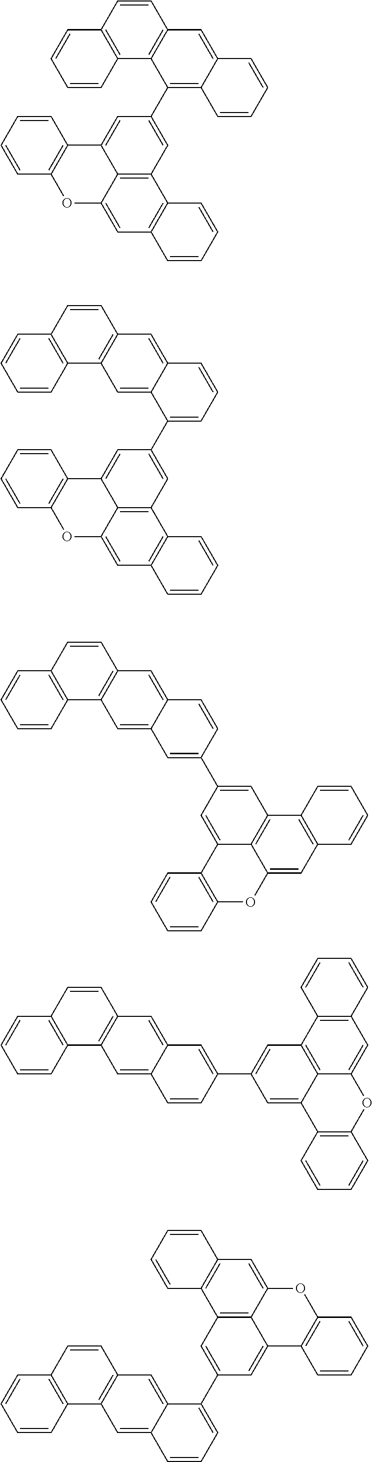 [SMILES: c1ccc2c(c1)Oc1cc3ccccc3c3cc(-c4c5ccccc5cc5ccc6ccccc6c45)cc-2c13 ; c1ccc2c(c1)Oc1cc3ccccc3c3cc(-c4cccc5cc6ccc7ccccc7c6cc45)cc-2c13 ; c1ccc2c(c1)Oc1cc3ccccc3c3cc(-c4ccc5cc6ccc7ccccc7c6cc5c4)cc-2c13 ; c1ccc2c(c1)Oc1cc3ccccc3c3cc(-c4ccc5cc6c(ccc7ccccc76)cc5c4)cc-2c13 ; c1ccc2c(c1)Oc1cc3ccccc3c3cc(-c4cccc5cc6c(ccc7ccccc76)cc45)cc-2c13]

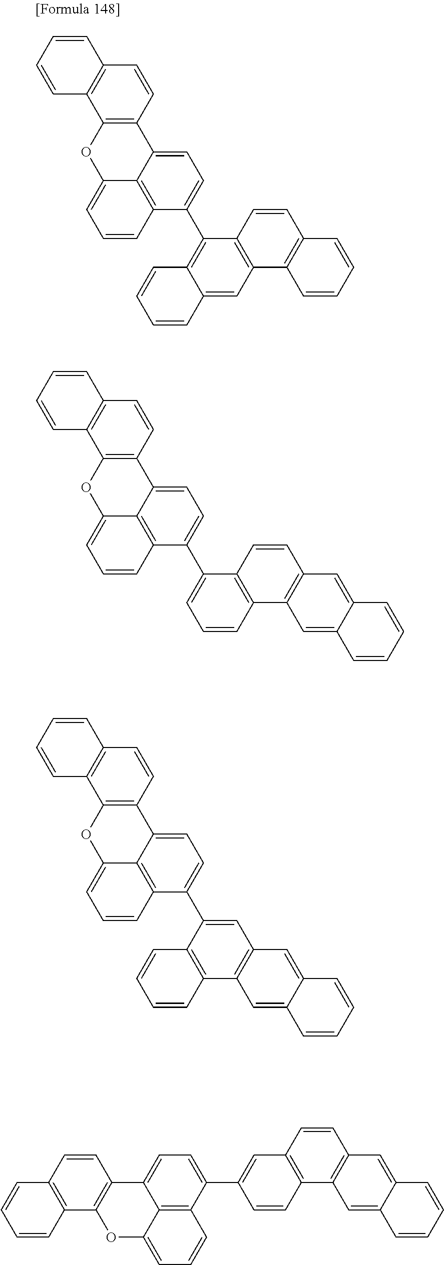 [SMILES: c1ccc2c3c(ccc2c1)-c1ccc(-c2c4ccccc4cc4c2ccc2ccccc24)c2cccc(c12)O3 ; c1ccc2cc3c(ccc4c(-c5ccc6c7c(cccc57)Oc5c-6ccc6ccccc56)cccc43)cc2c1 ; c1ccc2cc3c(cc(-c4ccc5c6c(cccc46)Oc4c-5ccc5ccccc45)c4ccccc43)cc2c1 ; c1ccc2cc3c(ccc4cc(-c5ccc6c7c(cccc57)Oc5c-6ccc6ccccc56)ccc43)cc2c1]

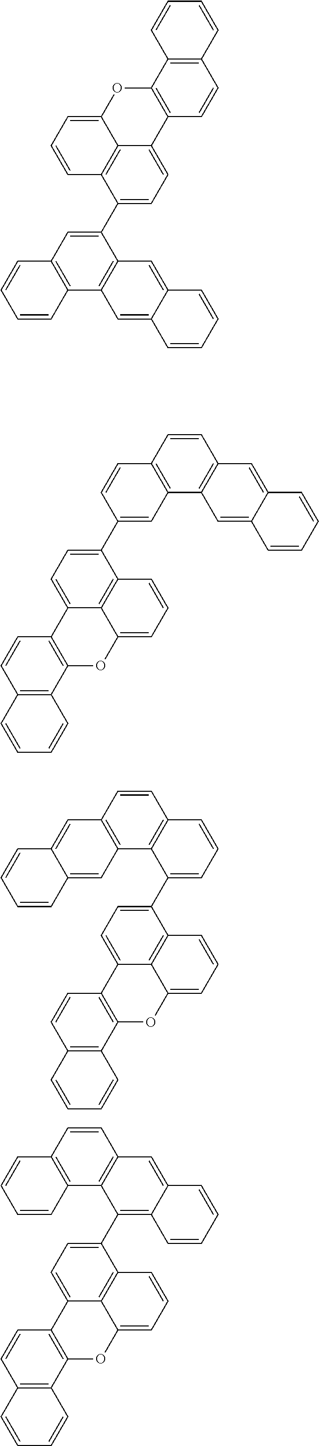

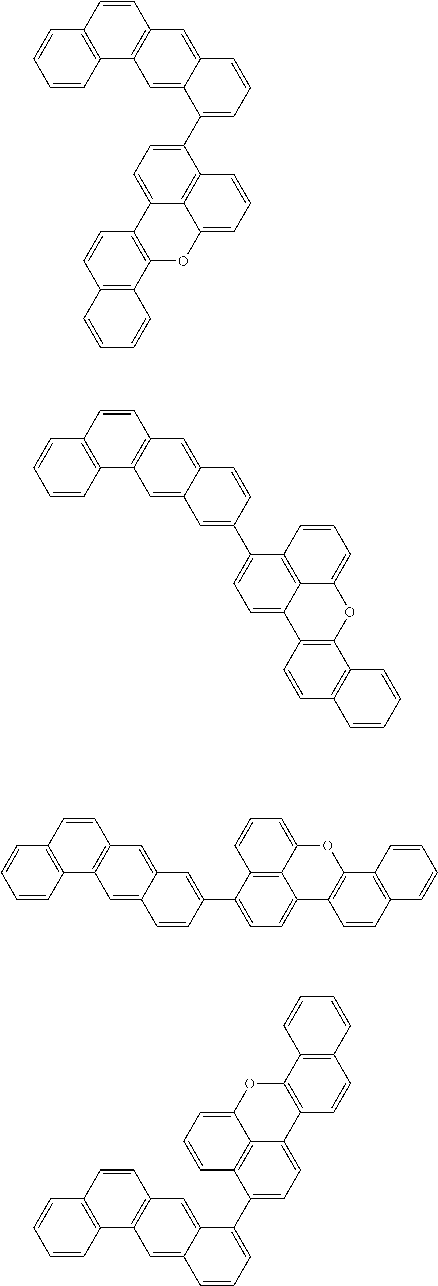 [SMILES: c1cc(-c2ccc3c4c(cccc24)Oc2c-3ccc3ccccc23)c2cc3c(ccc4ccccc43)cc2c1 ; c1ccc2c3c(ccc2c1)-c1ccc(-c2ccc4cc5ccc6ccccc6c5cc4c2)c2cccc(c12)O3 ; c1ccc2c3c(ccc2c1)-c1ccc(-c2ccc4cc5c(ccc6ccccc65)cc4c2)c2cccc(c12)O3 ; c1cc(-c2ccc3c4c(cccc24)Oc2c-3ccc3ccccc23)c2cc3ccc4ccccc4c3cc2c1]

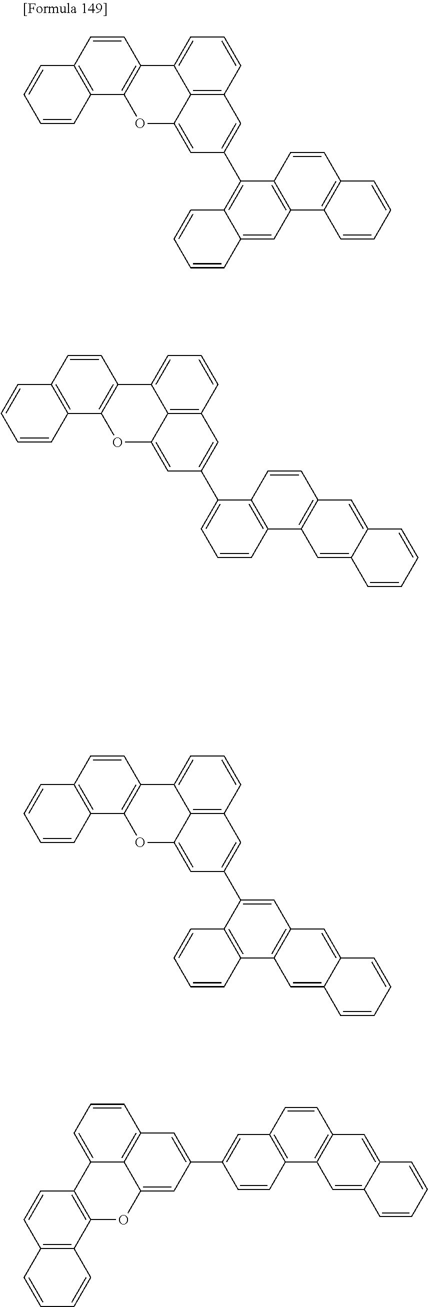 [SMILES: c1ccc2c3c(ccc2c1)-c1cccc2cc(-c4c5ccccc5cc5c4ccc4ccccc45)cc(c12)O3 ; c1ccc2cc3c(ccc4c(-c5cc6c7c(cccc7c5)-c5ccc7ccccc7c5O6)cccc43)cc2c1 ; c1ccc2cc3c(cc(-c4cc5c6c(cccc6c4)-c4ccc6ccccc6c4O5)c4ccccc43)cc2c1 ; c1ccc2cc3c(ccc4cc(-c5cc6c7c(cccc7c5)-c5ccc7ccccc7c5O6)ccc43)cc2c1]

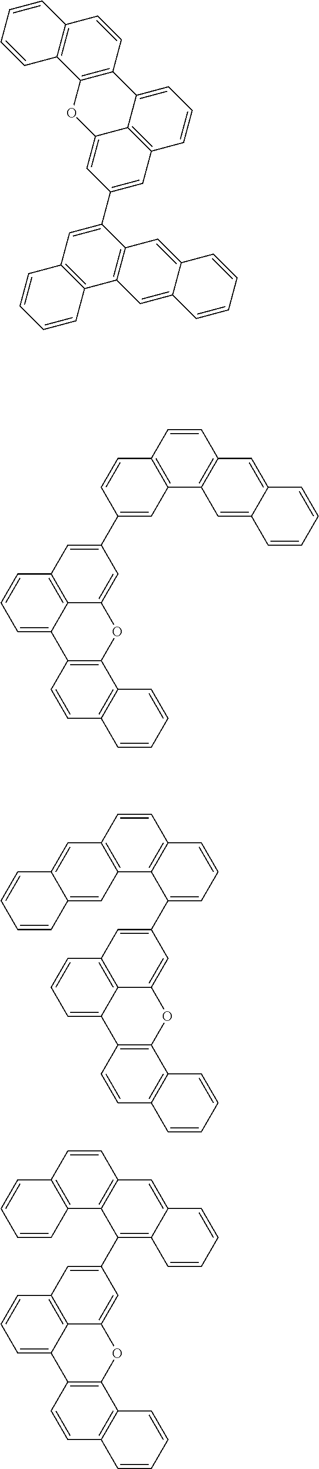 [SMILES: c1ccc2cc3c(cc2c1)c(-c1cc2c4c(cccc4c1)-c1ccc4ccccc4c1O2)cc1ccccc13 ; c1ccc2cc3c(ccc4ccc(-c5cc6c7c(cccc7c5)-c5ccc7ccccc7c5O6)cc43)cc2c1 ; c1ccc2cc3c(ccc4cccc(-c5cc6c7c(cccc7c5)-c5ccc7ccccc7c5O6)c43)cc2c1 ; c1ccc2c3c(ccc2c1)-c1cccc2cc(-c4c5ccccc5cc5ccc6ccccc6c45)cc(c12)O3]

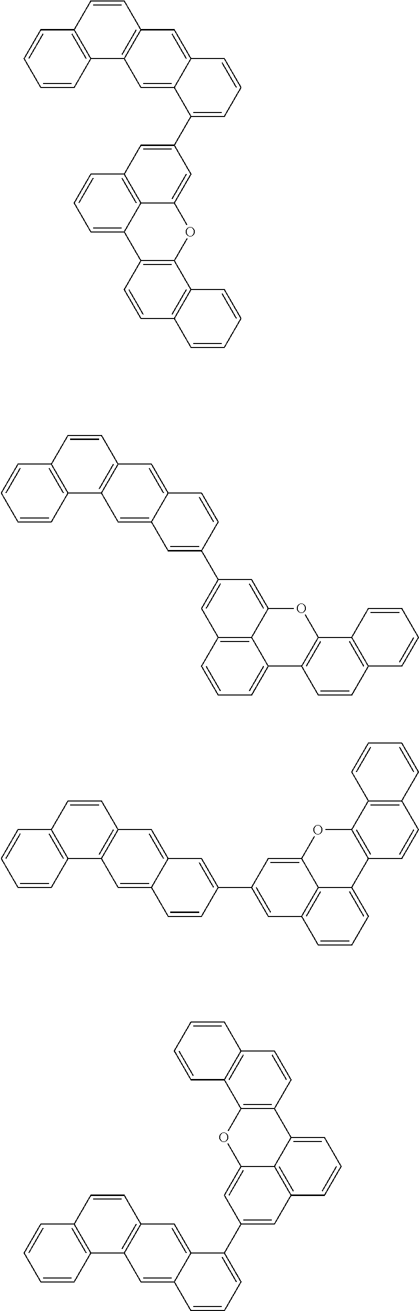 [SMILES: c1cc(-c2cc3c4c(cccc4c2)-c2ccc4ccccc4c2O3)c2cc3c(ccc4ccccc43)cc2c1 ; c1ccc2c3c(ccc2c1)-c1cccc2cc(-c4ccc5cc6ccc7ccccc7c6cc5c4)cc(c12)O3 ; c1ccc2c3c(ccc2c1)-c1cccc2cc(-c4ccc5cc6c(ccc7ccccc76)cc5c4)cc(c12)O3 ; c1cc(-c2cc3c4c(cccc4c2)-c2ccc4ccccc4c2O3)c2cc3ccc4ccccc4c3cc2c1]

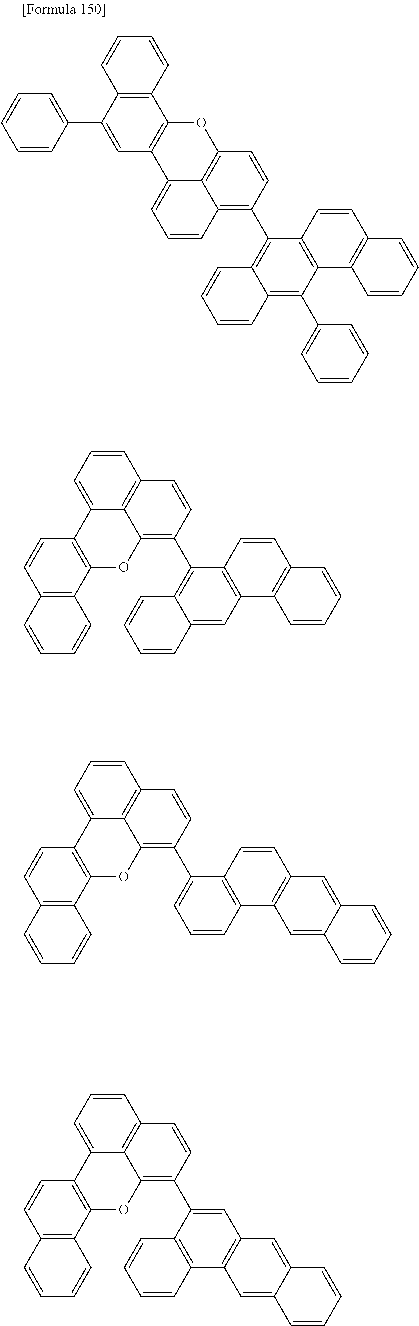 [SMILES: c1ccc(-c2cc3c(c4ccccc24)Oc2ccc(-c4c5ccccc5c(-c5ccccc5)c5c4ccc4ccccc45)c4cccc-3c24)cc1 ; c1ccc2c3c(ccc2c1)-c1cccc2ccc(-c4c5ccccc5cc5c4ccc4ccccc45)c(c12)O3 ; c1ccc2cc3c(ccc4c(-c5ccc6cccc7c6c5Oc5c-7ccc6ccccc56)cccc43)cc2c1 ; c1ccc2cc3c(cc(-c4ccc5cccc6c5c4Oc4c-6ccc5ccccc45)c4ccccc43)cc2c1]

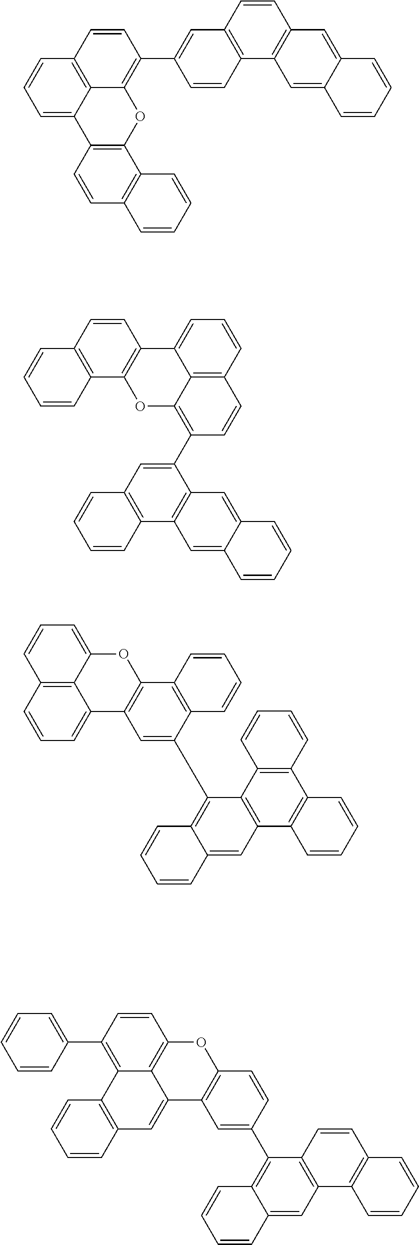 [SMILES: c1ccc2cc3c(ccc4cc(-c5ccc6cccc7c6c5Oc5c-7ccc6ccccc56)ccc43)cc2c1 ; c1ccc2cc3c(cc2c1)c(-c1ccc2cccc4c2c1Oc1c-4ccc2ccccc12)cc1ccccc13 ; c1ccc2c(-c3cc4c(c5ccccc35)Oc3cccc5cccc-4c35)c3c4ccccc4c4ccccc4c3cc2c1 ; c1ccc(-c2ccc3c4c(cc5ccccc5c24)-c2cc(-c4c5ccccc5cc5c4ccc4ccccc45)ccc2O3)cc1]

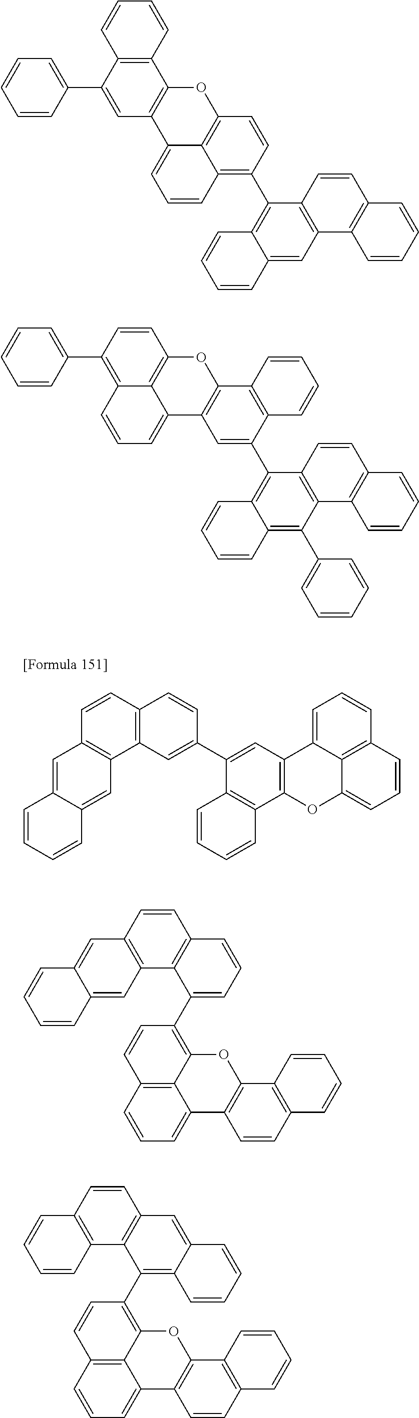 [SMILES: c1ccc(-c2cc3c(c4ccccc24)Oc2ccc(-c4c5ccccc5cc5c4ccc4ccccc45)c4cccc-3c24)cc1 ; c1ccc(-c2ccc3c4c(cccc24)-c2cc(-c4c5ccccc5c(-c5ccccc5)c5c4ccc4ccccc45)c4ccccc4c2O3)cc1 ; c1ccc2cc3c(ccc4ccc(-c5cc6c(c7ccccc57)Oc5cccc7cccc-6c57)cc43)cc2c1 ; c1ccc2cc3c(ccc4cccc(-c5ccc6cccc7c6c5Oc5c-7ccc6ccccc56)c43)cc2c1 ; c1ccc2c3c(ccc2c1)-c1cccc2ccc(-c4c5ccccc5cc5ccc6ccccc6c45)c(c12)O3]

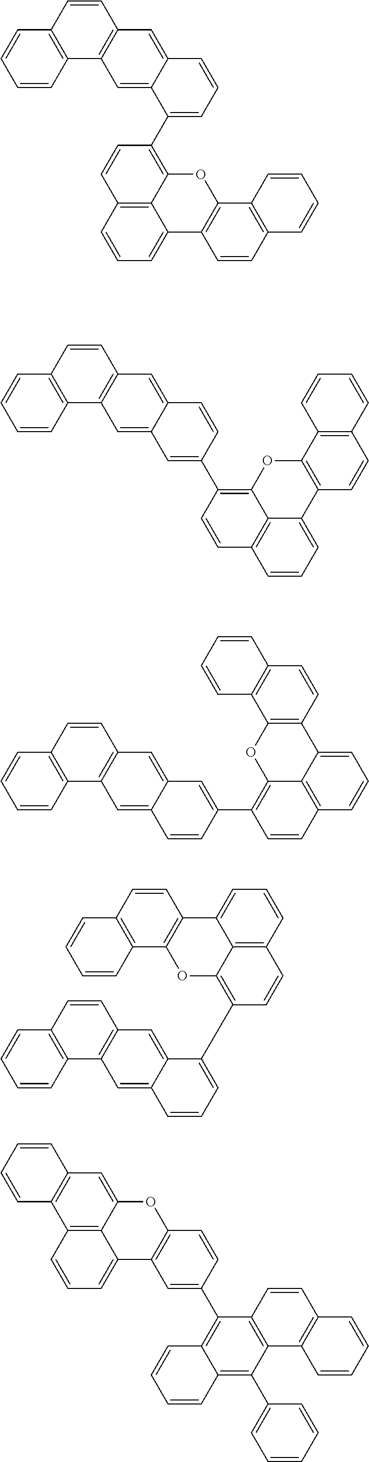 [SMILES: c1cc(-c2ccc3cccc4c3c2Oc2c-4ccc3ccccc23)c2cc3c(ccc4ccccc43)cc2c1 ; c1ccc2c3c(ccc2c1)-c1cccc2ccc(-c4ccc5cc6ccc7ccccc7c6cc5c4)c(c12)O3 ; c1ccc2c3c(ccc2c1)-c1cccc2ccc(-c4ccc5cc6c(ccc7ccccc76)cc5c4)c(c12)O3 ; c1cc(-c2ccc3cccc4c3c2Oc2c-4ccc3ccccc23)c2cc3ccc4ccccc4c3cc2c1 ; c1ccc(-c2c3ccccc3c(-c3ccc4c(c3)-c3cccc5c3c(cc3ccccc35)O4)c3ccc4ccccc4c23)cc1]

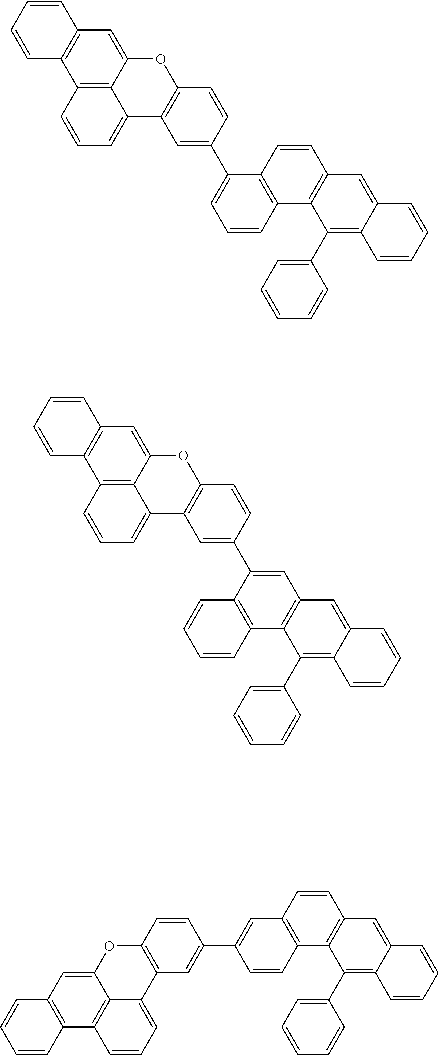 [SMILES: c1ccc(-c2c3ccccc3cc3ccc4c(-c5ccc6c(c5)-c5cccc7c5c(cc5ccccc57)O6)cccc4c23)cc1 ; c1ccc(-c2c3ccccc3cc3cc(-c4ccc5c(c4)-c4cccc6c4c(cc4ccccc46)O5)c4ccccc4c23)cc1 ; c1ccc(-c2c3ccccc3cc3ccc4cc(-c5ccc6c(c5)-c5cccc7c5c(cc5ccccc57)O6)ccc4c23)cc1]

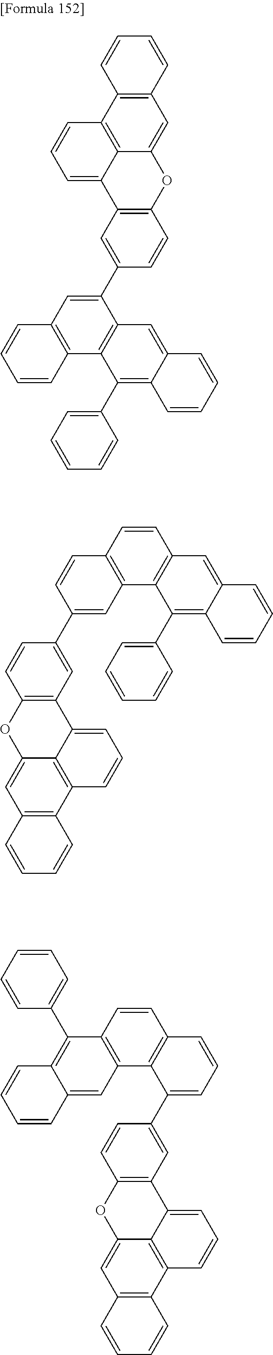 [SMILES: c1ccc(-c2c3ccccc3cc3c(-c4ccc5c(c4)-c4cccc6c4c(cc4ccccc46)O5)cc4ccccc4c23)cc1 ; c1ccc(-c2c3ccccc3cc3ccc4ccc(-c5ccc6c(c5)-c5cccc7c5c(cc5ccccc57)O6)cc4c23)cc1 ; c1ccc(-c2c3ccccc3cc3c2ccc2cccc(-c4ccc5c(c4)-c4cccc6c4c(cc4ccccc46)O5)c23)cc1]

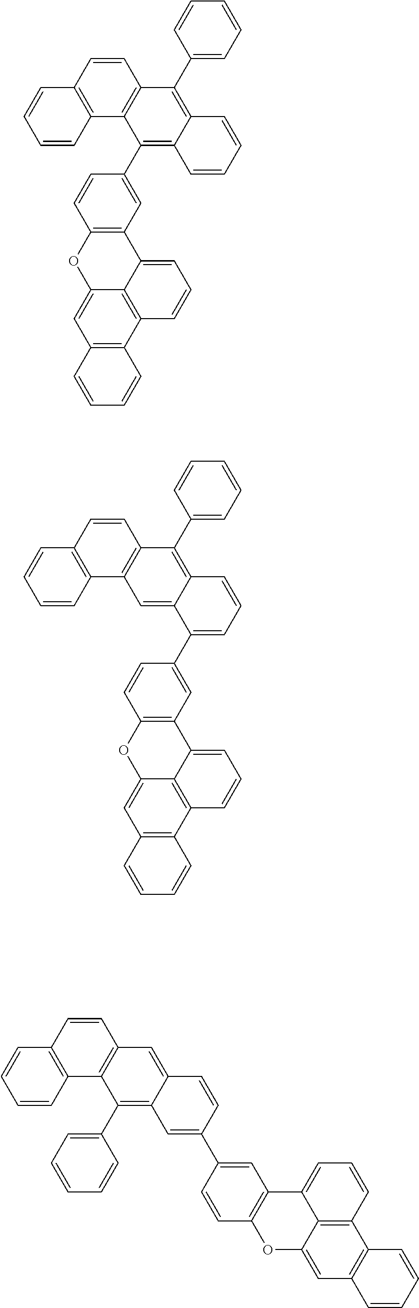 [SMILES: c1ccc(-c2c3ccccc3c(-c3ccc4c(c3)-c3cccc5c3c(cc3ccccc35)O4)c3c2ccc2ccccc23)cc1 ; c1ccc(-c2c3cccc(-c4ccc5c(c4)-c4cccc6c4c(cc4ccccc46)O5)c3cc3c2ccc2ccccc23)cc1 ; c1ccc(-c2c3cc(-c4ccc5c(c4)-c4cccc6c4c(cc4ccccc46)O5)ccc3cc3ccc4ccccc4c23)cc1]

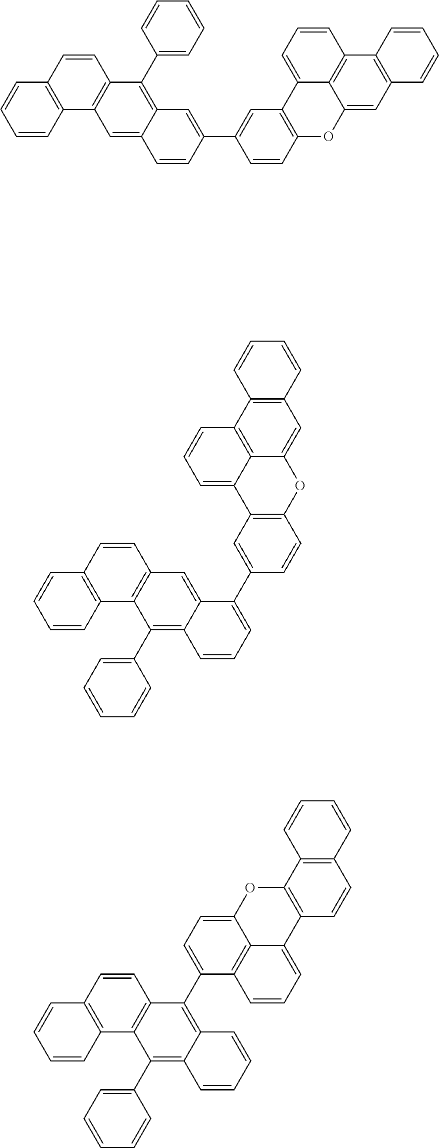 [SMILES: c1ccc(-c2c3cc(-c4ccc5c(c4)-c4cccc6c4c(cc4ccccc46)O5)ccc3cc3c2ccc2ccccc23)cc1 ; c1ccc(-c2c3cccc(-c4ccc5c(c4)-c4cccc6c4c(cc4ccccc46)O5)c3cc3ccc4ccccc4c23)cc1 ; c1ccc(-c2c3ccccc3c(-c3ccc4c5c(cccc35)-c3ccc5ccccc5c3O4)c3ccc4ccccc4c23)cc1]

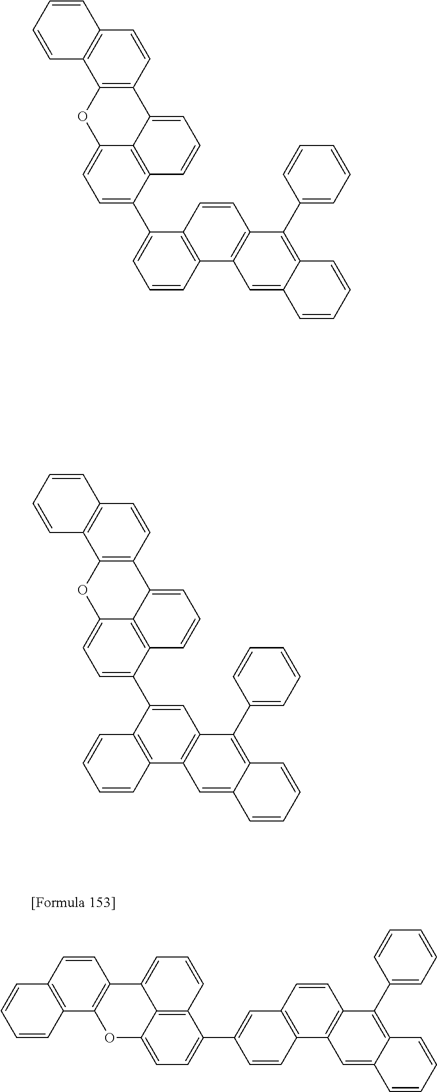 [SMILES: c1ccc(-c2c3ccccc3cc3c2ccc2c(-c4ccc5c6c(cccc46)-c4ccc6ccccc6c4O5)cccc23)cc1 ; c1ccc(-c2c3ccccc3cc3c2cc(-c2ccc4c5c(cccc25)-c2ccc5ccccc5c2O4)c2ccccc23)cc1 ; c1ccc(-c2c3ccccc3cc3c2ccc2cc(-c4ccc5c6c(cccc46)-c4ccc6ccccc6c4O5)ccc23)cc1]

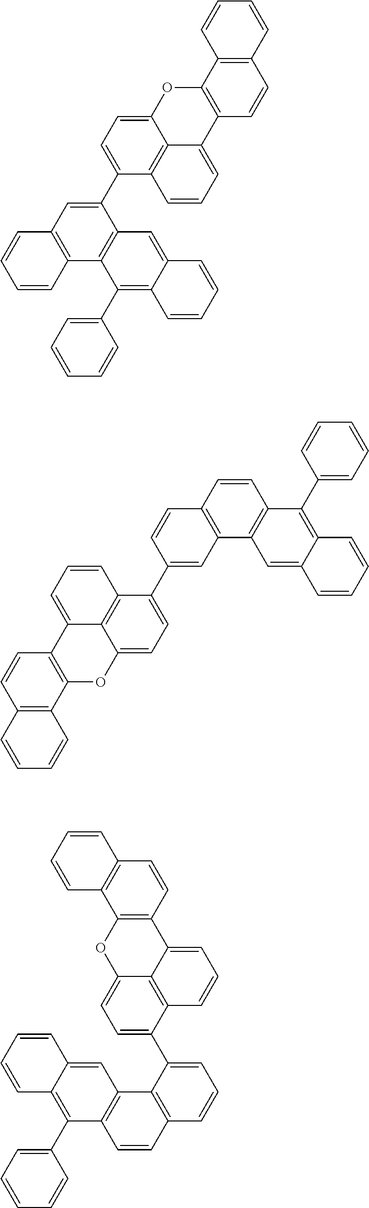 [SMILES: c1ccc(-c2c3ccccc3cc3c(-c4ccc5c6c(cccc46)-c4ccc6ccccc6c4O5)cc4ccccc4c23)cc1 ; c1ccc(-c2c3ccccc3cc3c2ccc2ccc(-c4ccc5c6c(cccc46)-c4ccc6ccccc6c4O5)cc23)cc1 ; c1ccc(-c2c3ccccc3cc3c2ccc2cccc(-c4ccc5c6c(cccc46)-c4ccc6ccccc6c4O5)c23)cc1]

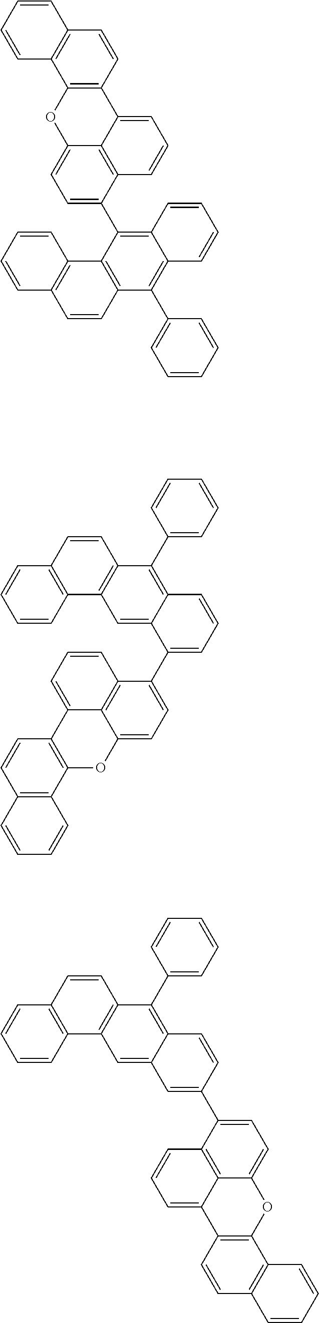 [SMILES: c1ccc(-c2c3ccccc3c(-c3ccc4c5c(cccc35)-c3ccc5ccccc5c3O4)c3c2ccc2ccccc23)cc1 ; c1ccc(-c2c3cccc(-c4ccc5c6c(cccc46)-c4ccc6ccccc6c4O5)c3cc3c2ccc2ccccc23)cc1 ; c1ccc(-c2c3ccc(-c4ccc5c6c(cccc46)-c4ccc6ccccc6c4O5)cc3cc3c2ccc2ccccc23)cc1]

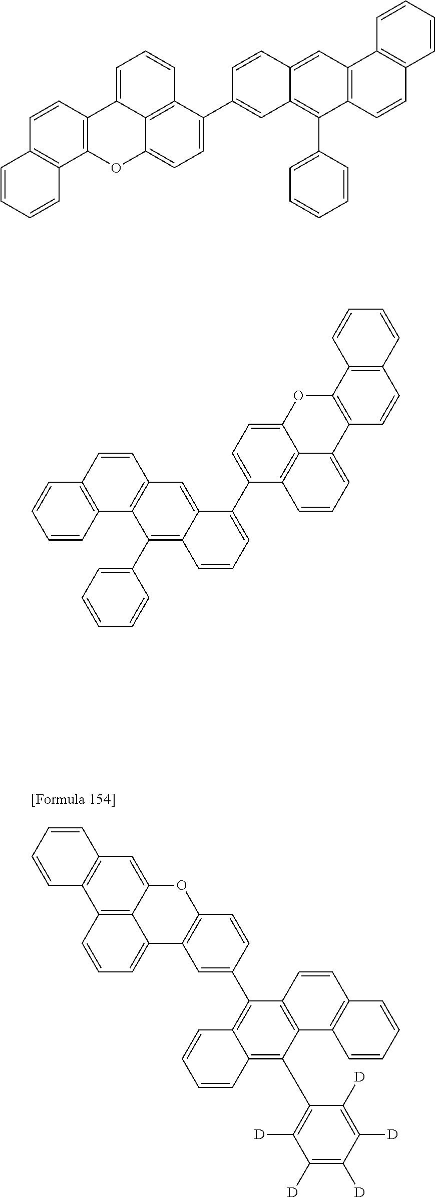 [SMILES: c1ccc(-c2c3cc(-c4ccc5c6c(cccc46)-c4ccc6ccccc6c4O5)ccc3cc3c2ccc2ccccc23)cc1 ; c1ccc(-c2c3cccc(-c4ccc5c6c(cccc46)-c4ccc6ccccc6c4O5)c3cc3ccc4ccccc4c23)cc1 ; [2H]c1c([2H])c([2H])c(-c2c3ccccc3c(-c3ccc4c(c3)-c3cccc5c3c(cc3ccccc35)O4)c3ccc4ccccc4c23)c([2H])c1[2H]]

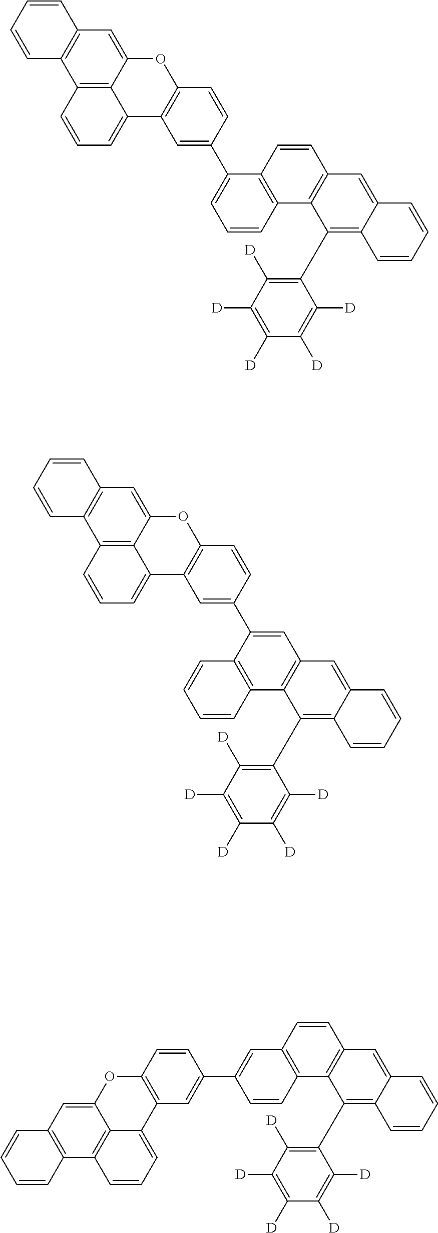 [SMILES: [2H]c1c([2H])c([2H])c(-c2c3ccccc3cc3ccc4c(-c5ccc6c(c5)-c5cccc7c5c(cc5ccccc57)O6)cccc4c23)c([2H])c1[2H] ; [2H]c1c([2H])c([2H])c(-c2c3ccccc3cc3cc(-c4ccc5c(c4)-c4cccc6c4c(cc4ccccc46)O5)c4ccccc4c23)c([2H])c1[2H] ; [2H]c1c([2H])c([2H])c(-c2c3ccccc3cc3ccc4cc(-c5ccc6c(c5)-c5cccc7c5c(cc5ccccc57)O6)ccc4c23)c([2H])c1[2H]]

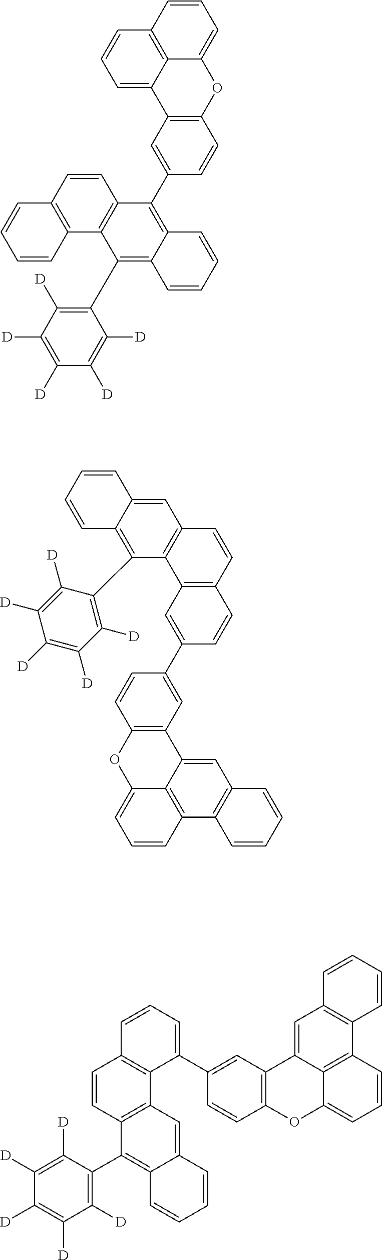 [SMILES: [2H]c1c([2H])c([2H])c(-c2c3ccccc3c(-c3ccc4c(c3)-c3cccc5cccc(c35)O4)c3ccc4ccccc4c23)c([2H])c1[2H] ; [2H]c1c([2H])c([2H])c(-c2c3ccccc3cc3ccc4ccc(-c5ccc6c(c5)-c5cc7ccccc7c7cccc(c57)O6)cc4c23)c([2H])c1[2H] ; [2H]c1c([2H])c([2H])c(-c2c3ccccc3cc3c2ccc2cccc(-c4ccc5c(c4)-c4cc6ccccc6c6cccc(c46)O5)c23)c([2H])c1[2H]]

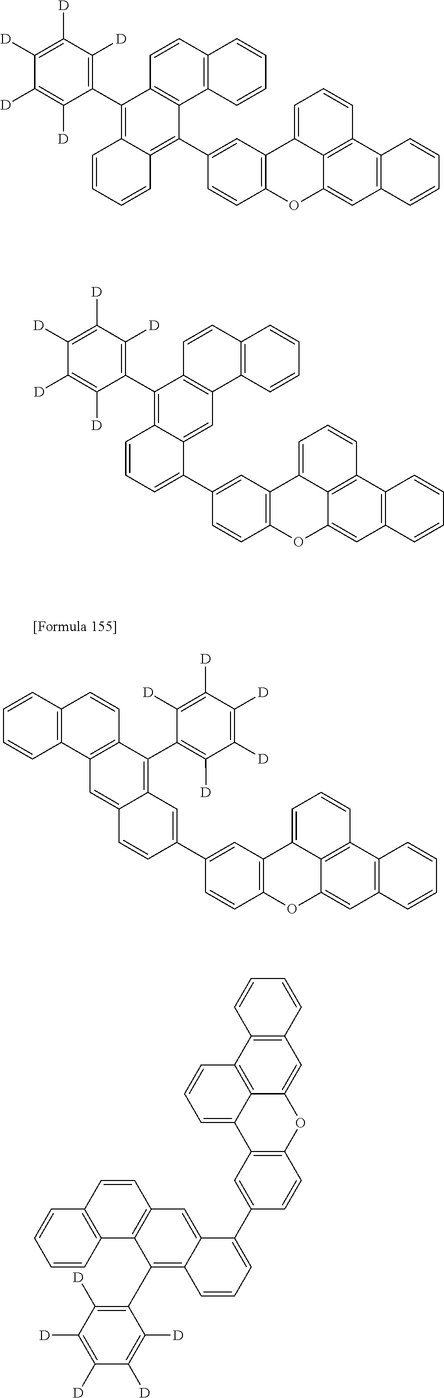 [SMILES: [2H]c1c([2H])c([2H])c(-c2c3ccccc3c(-c3ccc4c(c3)-c3cccc5c3c(cc3ccccc35)O4)c3c2ccc2ccccc23)c([2H])c1[2H] ; [2H]c1c([2H])c([2H])c(-c2c3cccc(-c4ccc5c(c4)-c4cccc6c4c(cc4ccccc46)O5)c3cc3c2ccc2ccccc23)c([2H])c1[2H] ; [2H]c1c([2H])c([2H])c(-c2c3cc(-c4ccc5c(c4)-c4cccc6c4c(cc4ccccc46)O5)ccc3cc3c2ccc2ccccc23)c([2H])c1[2H] ; [2H]c1c([2H])c([2H])c(-c2c3cccc(-c4ccc5c(c4)-c4cccc6c4c(cc4ccccc46)O5)c3cc3ccc4ccccc4c23)c([2H])c1[2H]]

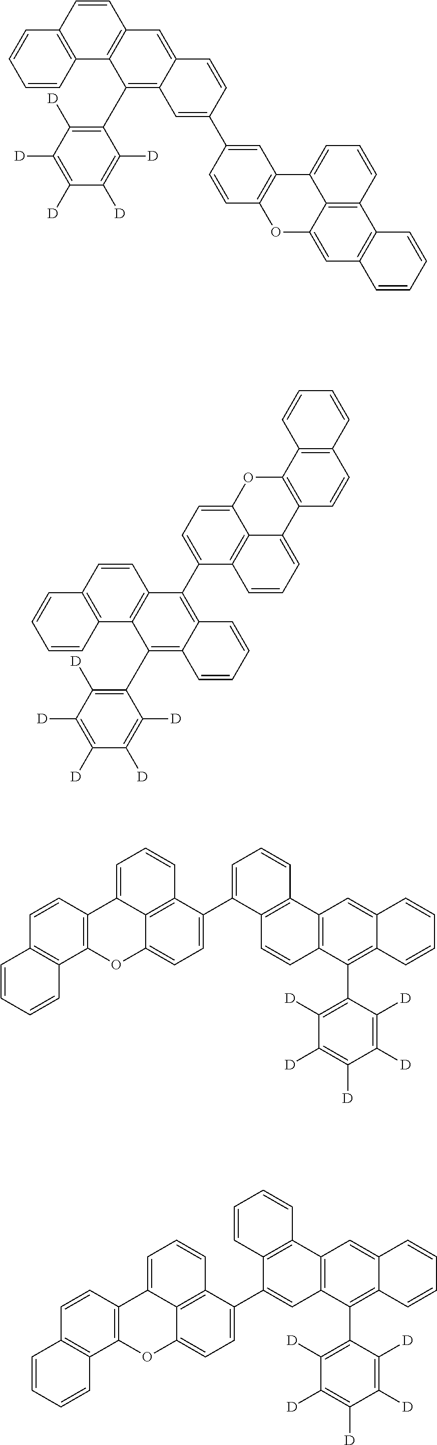 [SMILES: [2H]c1c([2H])c([2H])c(-c2c3cc(-c4ccc5c(c4)-c4cccc6c4c(cc4ccccc46)O5)ccc3cc3ccc4ccccc4c23)c([2H])c1[2H] ; [2H]c1c([2H])c([2H])c(-c2c3ccccc3c(-c3ccc4c5c(cccc35)-c3ccc5ccccc5c3O4)c3ccc4ccccc4c23)c([2H])c1[2H] ; [2H]c1c([2H])c([2H])c(-c2c3ccccc3cc3c2ccc2c(-c4ccc5c6c(cccc46)-c4ccc6ccccc6c4O5)cccc23)c([2H])c1[2H] ; [2H]c1c([2H])c([2H])c(-c2c3ccccc3cc3c2cc(-c2ccc4c5c(cccc25)-c2ccc5ccccc5c2O4)c2ccccc23)c([2H])c1[2H]]

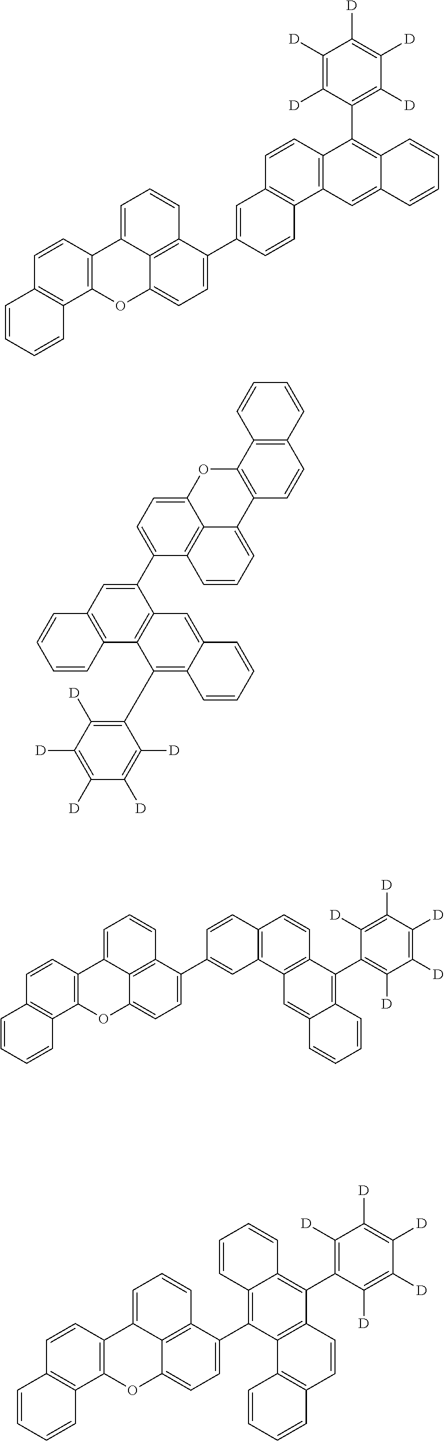 [SMILES: [2H]c1c([2H])c([2H])c(-c2c3ccccc3cc3c2ccc2cc(-c4ccc5c6c(cccc46)-c4ccc6ccccc6c4O5)ccc23)c([2H])c1[2H] ; [2H]c1c([2H])c([2H])c(-c2c3ccccc3cc3c(-c4ccc5c6c(cccc46)-c4ccc6ccccc6c4O5)cc4ccccc4c23)c([2H])c1[2H] ; [2H]c1c([2H])c([2H])c(-c2c3ccccc3cc3c2ccc2ccc(-c4ccc5c6c(cccc46)-c4ccc6ccccc6c4O5)cc23)c([2H])c1[2H] ; [2H]c1c([2H])c([2H])c(-c2c3ccccc3c(-c3ccc4c5c(cccc35)-c3ccc5ccccc5c3O4)c3c2ccc2ccccc23)c([2H])c1[2H]]

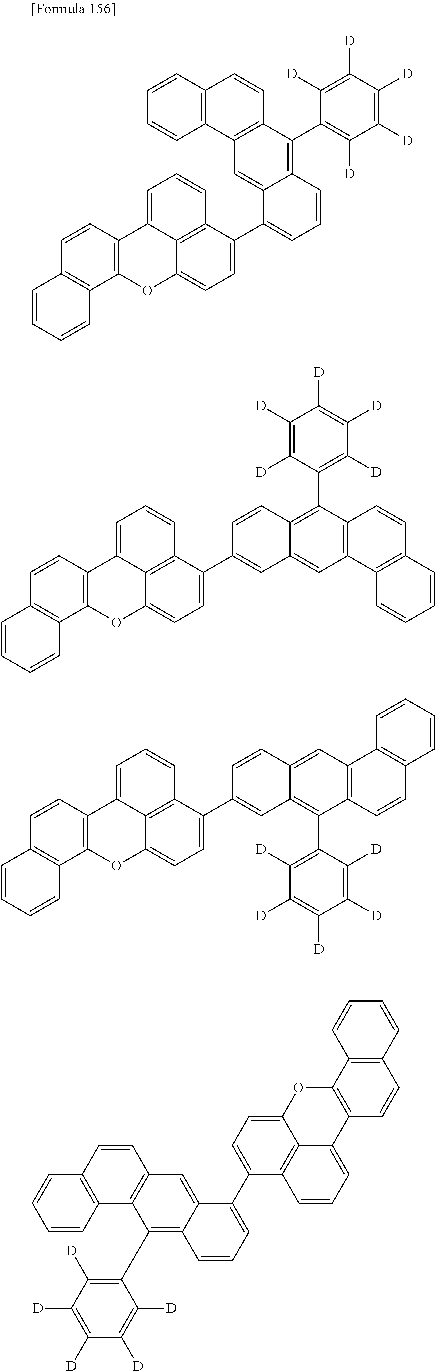 [SMILES: [2H]c1c([2H])c([2H])c(-c2c3cccc(-c4ccc5c6c(cccc46)-c4ccc6ccccc6c4O5)c3cc3c2ccc2ccccc23)c([2H])c1[2H] ; [2H]c1c([2H])c([2H])c(-c2c3ccc(-c4ccc5c6c(cccc46)-c4ccc6ccccc6c4O5)cc3cc3c2ccc2ccccc23)c([2H])c1[2H] ; [2H]c1c([2H])c([2H])c(-c2c3cc(-c4ccc5c6c(cccc46)-c4ccc6ccccc6c4O5)ccc3cc3c2ccc2ccccc23)c([2H])c1[2H] ; [2H]c1c([2H])c([2H])c(-c2c3cccc(-c4ccc5c6c(cccc46)-c4ccc6ccccc6c4O5)c3cc3ccc4ccccc4c23)c([2H])c1[2H]]

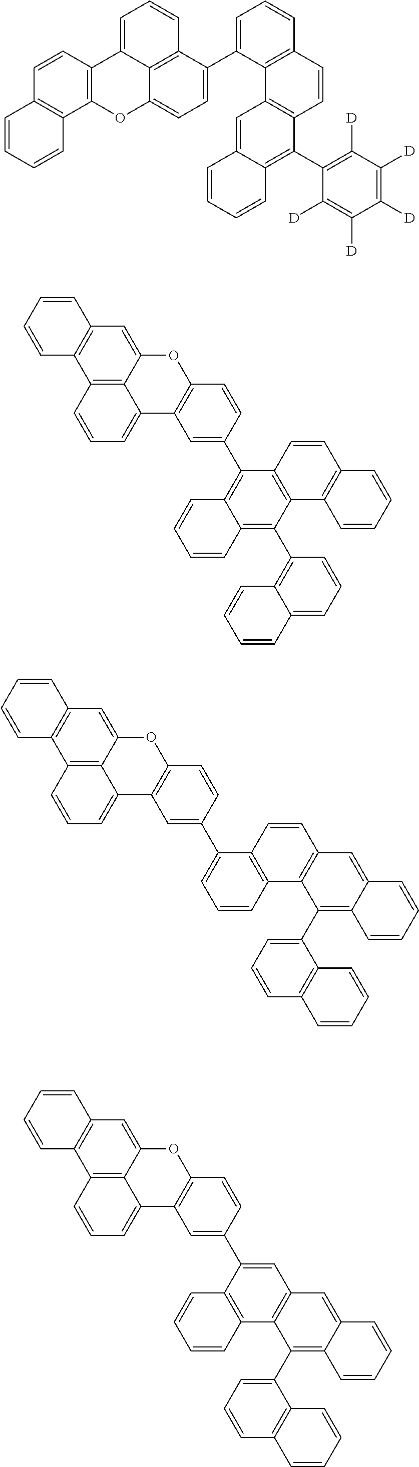 [SMILES: [2H]c1c([2H])c([2H])c(-c2c3ccccc3cc3c2ccc2cccc(-c4ccc5c6c(cccc46)-c4ccc6ccccc6c4O5)c23)c([2H])c1[2H] ; c1ccc2c(-c3c4ccccc4c(-c4ccc5c(c4)-c4cccc6c4c(cc4ccccc46)O5)c4ccc5ccccc5c34)cccc2c1 ; c1ccc2c(-c3c4ccccc4cc4ccc5c(-c6ccc7c(c6)-c6cccc8c6c(cc6ccccc68)O7)cccc5c34)cccc2c1 ; c1ccc2c(-c3c4ccccc4cc4cc(-c5ccc6c(c5)-c5cccc7c5c(cc5ccccc57)O6)c5ccccc5c34)cccc2c1]

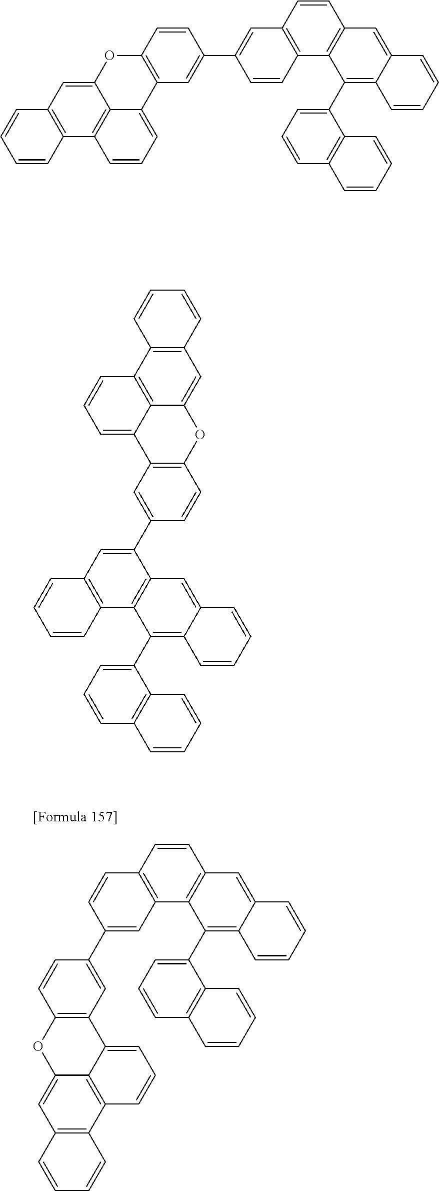 [SMILES: c1ccc2c(-c3c4ccccc4cc4ccc5cc(-c6ccc7c(c6)-c6cccc8c6c(cc6ccccc68)O7)ccc5c34)cccc2c1 ; c1ccc2c(-c3c4ccccc4cc4c(-c5ccc6c(c5)-c5cccc7c5c(cc5ccccc57)O6)cc5ccccc5c34)cccc2c1 ; c1ccc2c(-c3c4ccccc4cc4ccc5ccc(-c6ccc7c(c6)-c6cccc8c6c(cc6ccccc68)O7)cc5c34)cccc2c1]

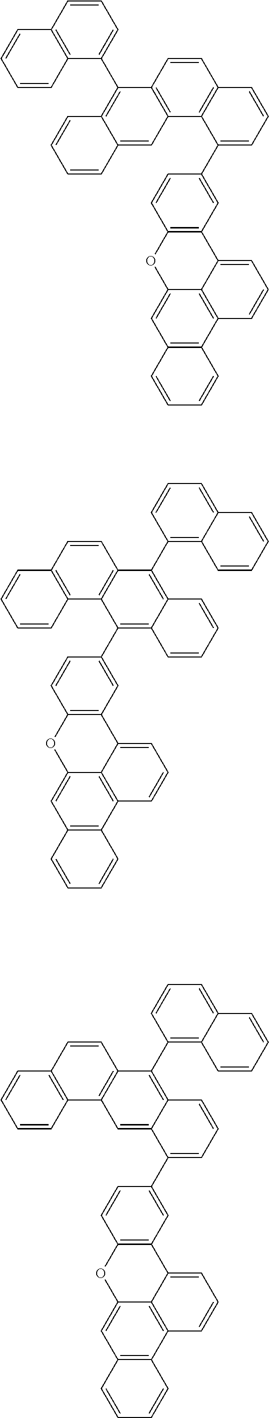 [SMILES: c1ccc2c(-c3c4ccccc4cc4c3ccc3cccc(-c5ccc6c(c5)-c5cccc7c5c(cc5ccccc57)O6)c34)cccc2c1 ; c1ccc2c(-c3c4ccccc4c(-c4ccc5c(c4)-c4cccc6c4c(cc4ccccc46)O5)c4c3ccc3ccccc34)cccc2c1 ; c1ccc2c(-c3c4cccc(-c5ccc6c(c5)-c5cccc7c5c(cc5ccccc57)O6)c4cc4c3ccc3ccccc34)cccc2c1]

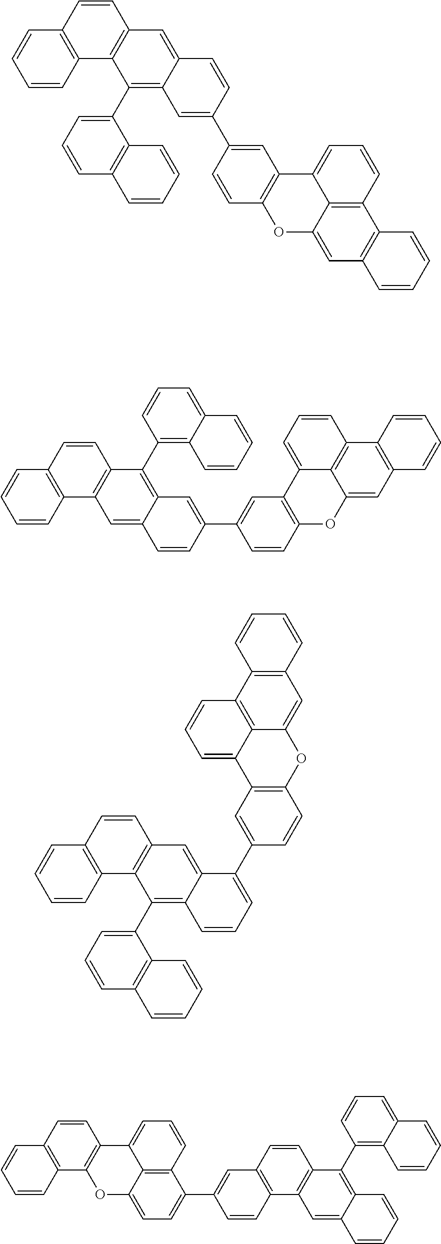 [SMILES: c1ccc2c(-c3c4cc(-c5ccc6c(c5)-c5cccc7c5c(cc5ccccc57)O6)ccc4cc4ccc5ccccc5c34)cccc2c1 ; c1ccc2c(-c3c4cc(-c5ccc6c(c5)-c5cccc7c5c(cc5ccccc57)O6)ccc4cc4c3ccc3ccccc34)cccc2c1 ; c1ccc2c(-c3c4cccc(-c5ccc6c(c5)-c5cccc7c5c(cc5ccccc57)O6)c4cc4ccc5ccccc5c34)cccc2c1 ; c1ccc2c(-c3c4ccccc4cc4c3ccc3cc(-c5ccc6c7c(cccc57)-c5ccc7ccccc7c5O6)ccc34)cccc2c1]

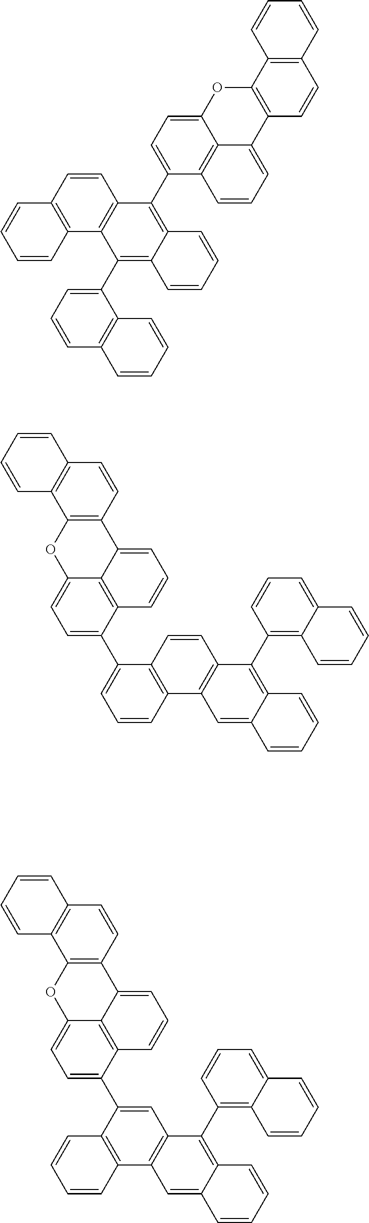 [SMILES: c1ccc2c(-c3c4ccccc4c(-c4ccc5c6c(cccc46)-c4ccc6ccccc6c4O5)c4ccc5ccccc5c34)cccc2c1 ; c1ccc2c(-c3c4ccccc4cc4c3ccc3c(-c5ccc6c7c(cccc57)-c5ccc7ccccc7c5O6)cccc34)cccc2c1 ; c1ccc2c(-c3c4ccccc4cc4c3cc(-c3ccc5c6c(cccc36)-c3ccc6ccccc6c3O5)c3ccccc34)cccc2c1]

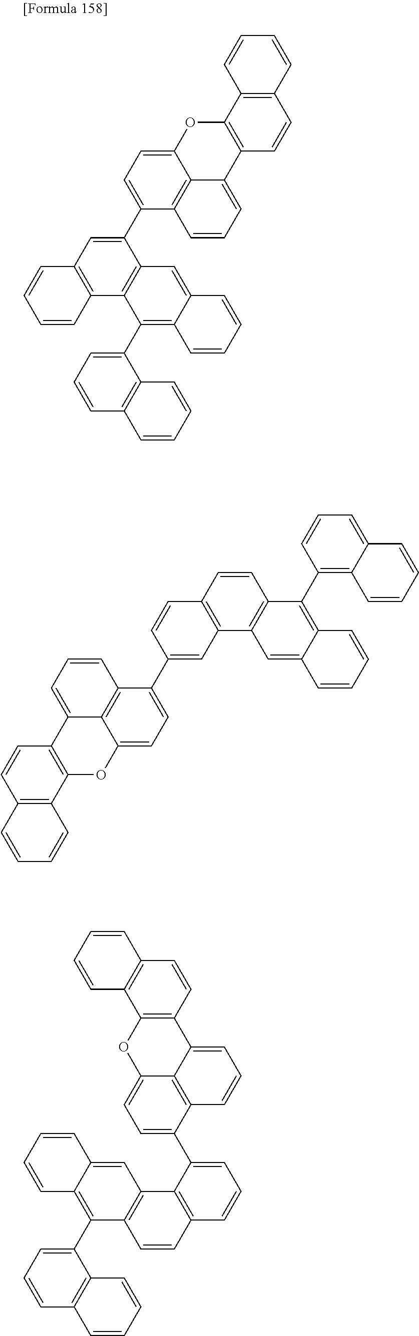 [SMILES: c1ccc2c(-c3c4ccccc4cc4c(-c5ccc6c7c(cccc57)-c5ccc7ccccc7c5O6)cc5ccccc5c34)cccc2c1 ; c1ccc2c(-c3c4ccccc4cc4c3ccc3ccc(-c5ccc6c7c(cccc57)-c5ccc7ccccc7c5O6)cc34)cccc2c1 ; c1ccc2c(-c3c4ccccc4cc4c3ccc3cccc(-c5ccc6c7c(cccc57)-c5ccc7ccccc7c5O6)c34)cccc2c1]

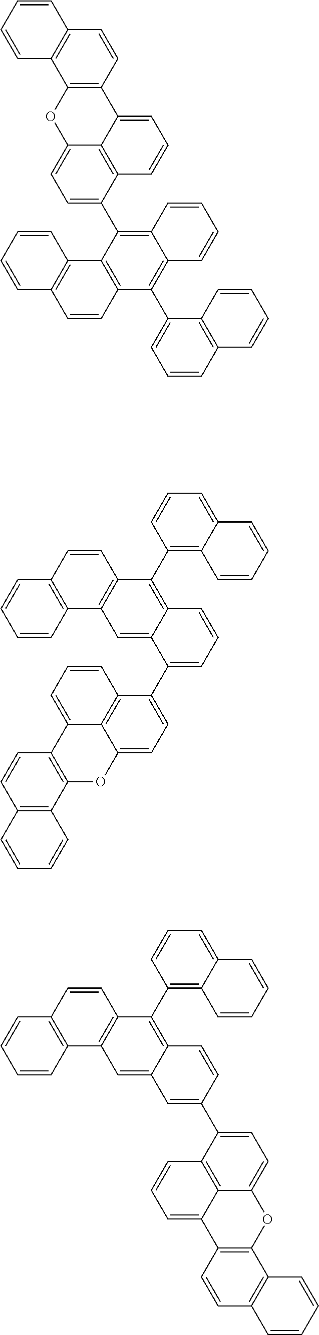 [SMILES: c1ccc2c(-c3c4ccccc4c(-c4ccc5c6c(cccc46)-c4ccc6ccccc6c4O5)c4c3ccc3ccccc34)cccc2c1 ; c1ccc2c(-c3c4cccc(-c5ccc6c7c(cccc57)-c5ccc7ccccc7c5O6)c4cc4c3ccc3ccccc34)cccc2c1 ; c1ccc2c(-c3c4ccc(-c5ccc6c7c(cccc57)-c5ccc7ccccc7c5O6)cc4cc4c3ccc3ccccc34)cccc2c1]

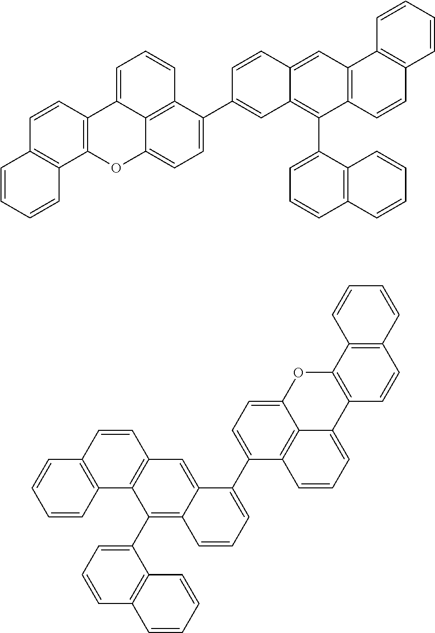 [SMILES: c1ccc2c(-c3c4cc(-c5ccc6c7c(cccc57)-c5ccc7ccccc7c5O6)ccc4cc4c3ccc3ccccc34)cccc2c1 ; c1ccc2c(-c3c4cccc(-c5ccc6c7c(cccc57)-c5ccc7ccccc7c5O6)c4cc4ccc5ccccc5c34)cccc2c1]

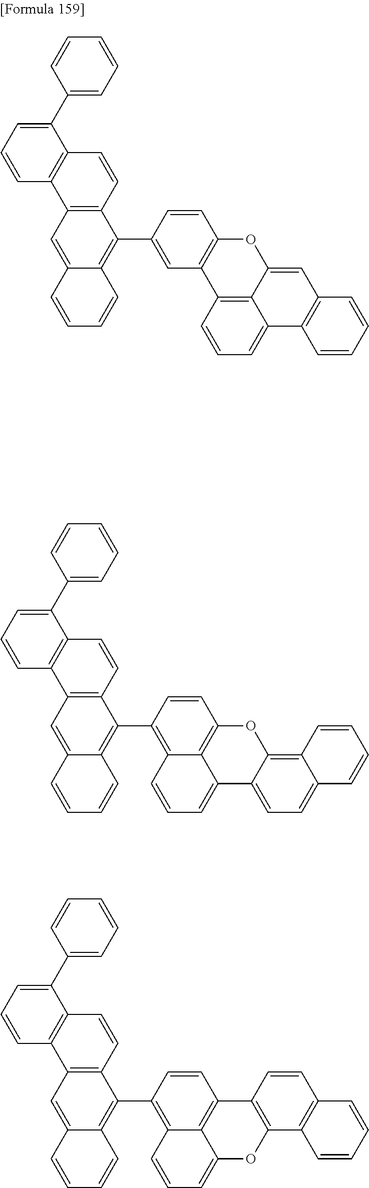 [SMILES: c1ccc(-c2cccc3c2ccc2c(-c4ccc5c(c4)-c4cccc6c4c(cc4ccccc46)O5)c4ccccc4cc23)cc1 ; c1ccc(-c2cccc3c2ccc2c(-c4ccc5c6c(cccc46)-c4ccc6ccccc6c4O5)c4ccccc4cc23)cc1 ; c1ccc(-c2cccc3c2ccc2c(-c4ccc5c6c(cccc46)Oc4c-5ccc5ccccc45)c4ccccc4cc23)cc1]

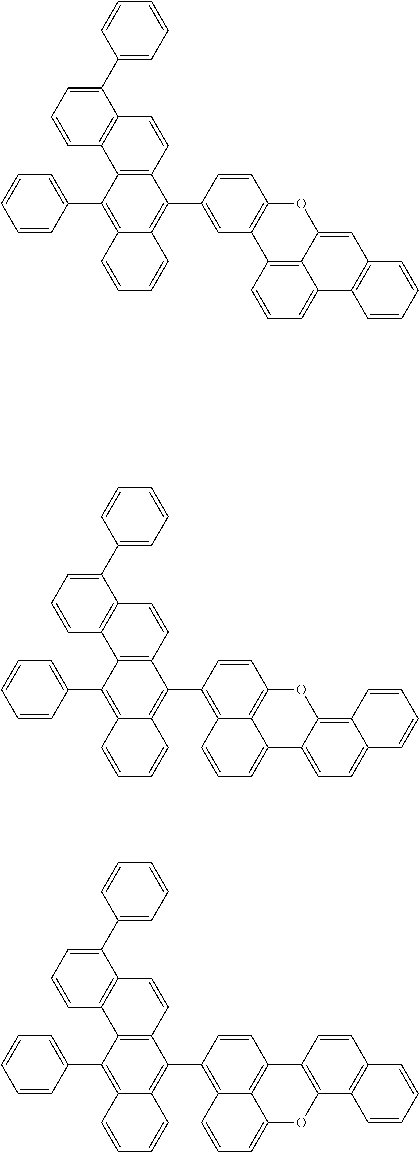 [SMILES: c1ccc(-c2cccc3c2ccc2c(-c4ccc5c(c4)-c4cccc6c4c(cc4ccccc46)O5)c4ccccc4c(-c4ccccc4)c23)cc1 ; c1ccc(-c2cccc3c2ccc2c(-c4ccc5c6c(cccc46)-c4ccc6ccccc6c4O5)c4ccccc4c(-c4ccccc4)c23)cc1 ; c1ccc(-c2cccc3c2ccc2c(-c4ccc5c6c(cccc46)Oc4c-5ccc5ccccc45)c4ccccc4c(-c4ccccc4)c23)cc1]

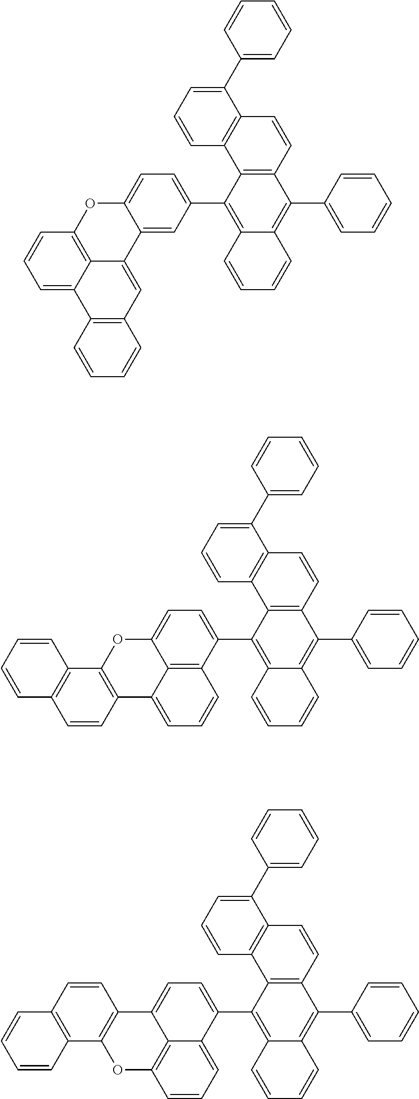 [SMILES: c1ccc(-c2cccc3c2ccc2c(-c4ccccc4)c4ccccc4c(-c4ccc5c(c4)-c4cc6ccccc6c6cccc(c46)O5)c23)cc1 ; c1ccc(-c2cccc3c2ccc2c(-c4ccccc4)c4ccccc4c(-c4ccc5c6c(cccc46)-c4ccc6ccccc6c4O5)c23)cc1 ; c1ccc(-c2cccc3c2ccc2c(-c4ccccc4)c4ccccc4c(-c4ccc5c6c(cccc46)Oc4c-5ccc5ccccc45)c23)cc1]

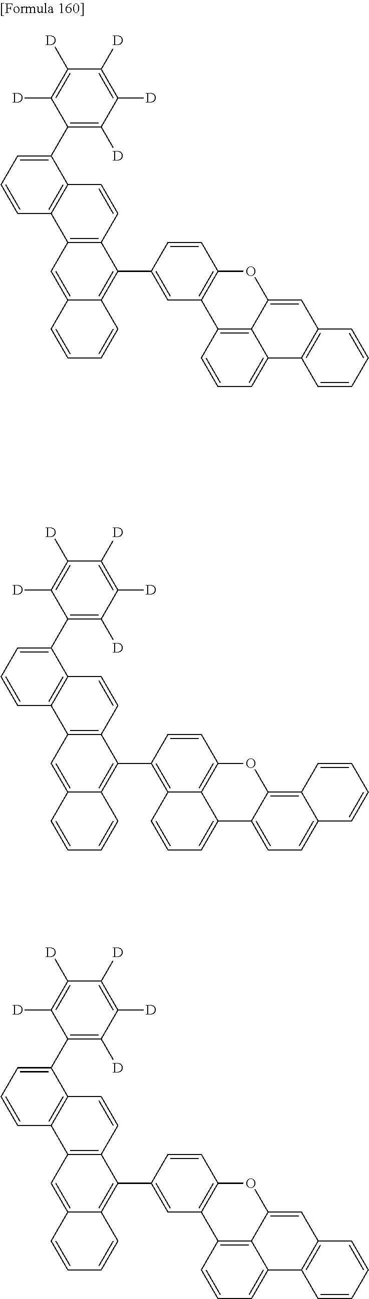 [SMILES: [2H]c1c([2H])c([2H])c(-c2cccc3c2ccc2c(-c4ccc5c(c4)-c4cccc6c4c(cc4ccccc46)O5)c4ccccc4cc23)c([2H])c1[2H] ; [2H]c1c([2H])c([2H])c(-c2cccc3c2ccc2c(-c4ccc5c6c(cccc46)-c4ccc6ccccc6c4O5)c4ccccc4cc23)c([2H])c1[2H] ; [2H]c1c([2H])c([2H])c(-c2cccc3c2ccc2c(-c4ccc5c(c4)-c4cccc6c4c(cc4ccccc46)O5)c4ccccc4cc23)c([2H])c1[2H]]

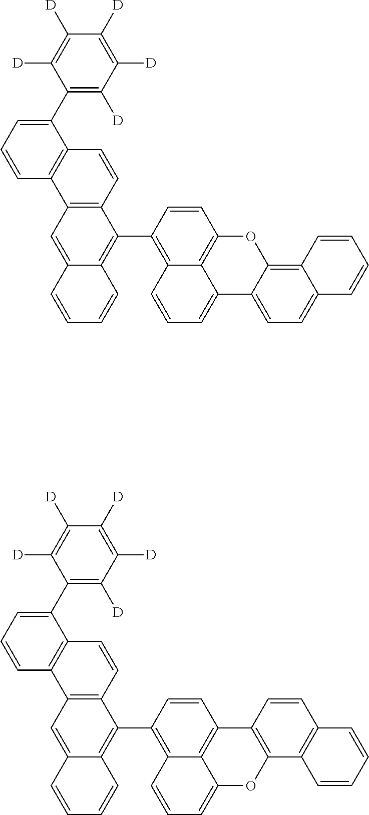 [SMILES: [2H]c1c([2H])c([2H])c(-c2cccc3c2ccc2c(-c4ccc5c6c(cccc46)-c4ccc6ccccc6c4O5)c4ccccc4cc23)c([2H])c1[2H] ; [2H]c1c([2H])c([2H])c(-c2cccc3c2ccc2c(-c4ccc5c6c(cccc46)Oc4c-5ccc5ccccc45)c4ccccc4cc23)c([2H])c1[2H]]

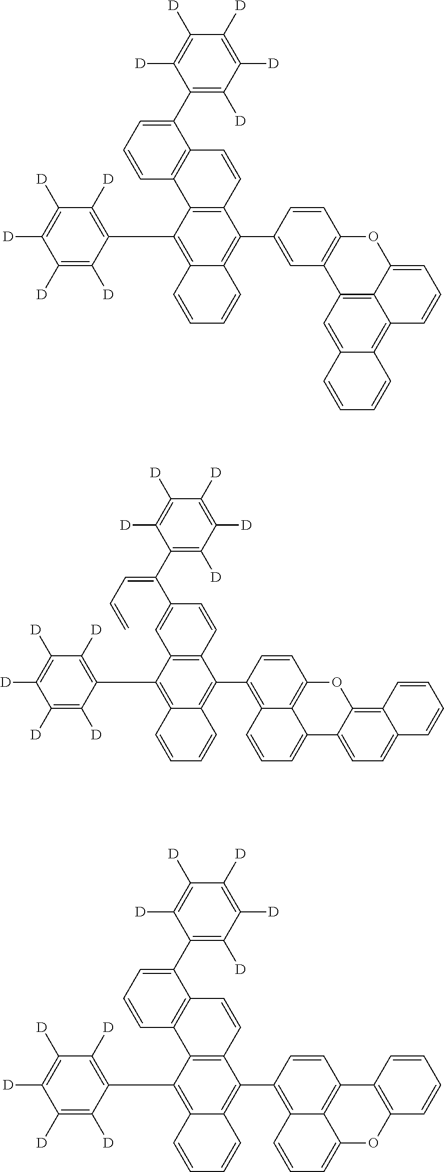 [SMILES: [2H]c1c([2H])c([2H])c(-c2cccc3c2ccc2c(-c4ccc5c(c4)-c4cc6ccccc6c6cccc(c46)O5)c4ccccc4c(-c4c([2H])c([2H])c([2H])c([2H])c4[2H])c23)c([2H])c1[2H] ; [2H]c1c([2H])c([2H])c(/C(=C/C=C)c2ccc3c(-c4ccc5c6c(cccc46)-c4ccc6ccccc6c4O5)c4ccccc4c(-c4c([2H])c([2H])c([2H])c([2H])c4[2H])c3c2)c([2H])c1[2H] ; [2H]c1c([2H])c([2H])c(-c2cccc3c2ccc2c(-c4ccc5c6c(cccc46)Oc4ccccc4-5)c4ccccc4c(-c4c([2H])c([2H])c([2H])c([2H])c4[2H])c23)c([2H])c1[2H]]

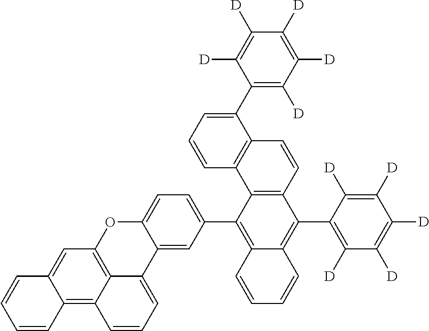 [SMILES: [2H]c1c([2H])c([2H])c(-c2cccc3c2ccc2c(-c4c([2H])c([2H])c([2H])c([2H])c4[2H])c4ccccc4c(-c4ccc5c(c4)-c4cccc6c4c(cc4ccccc46)O5)c23)c([2H])c1[2H]]

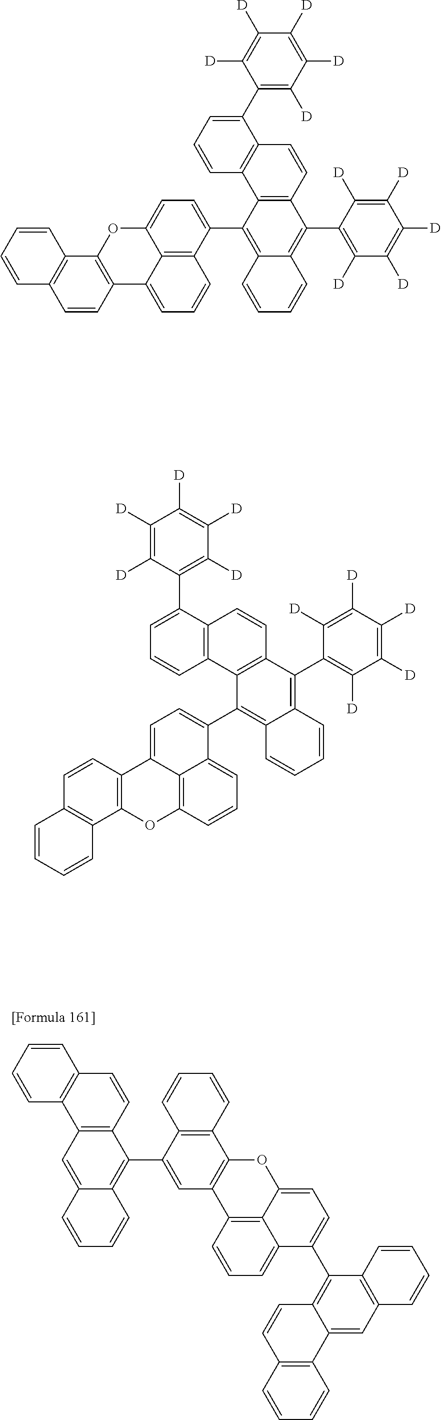 [SMILES: [2H]c1c([2H])c([2H])c(-c2cccc3c2ccc2c(-c4c([2H])c([2H])c([2H])c([2H])c4[2H])c4ccccc4c(-c4ccc5c6c(cccc46)-c4ccc6ccccc6c4O5)c23)c([2H])c1[2H] ; [2H]c1c([2H])c([2H])c(-c2cccc3c2ccc2c(-c4c([2H])c([2H])c([2H])c([2H])c4[2H])c4ccccc4c(-c4ccc5c6c(cccc46)Oc4c-5ccc5ccccc45)c23)c([2H])c1[2H] ; c1ccc2c(-c3cc4c(c5ccccc35)Oc3ccc(-c5c6ccccc6cc6c5ccc5ccccc56)c5cccc-4c35)c3ccc4ccccc4c3cc2c1]

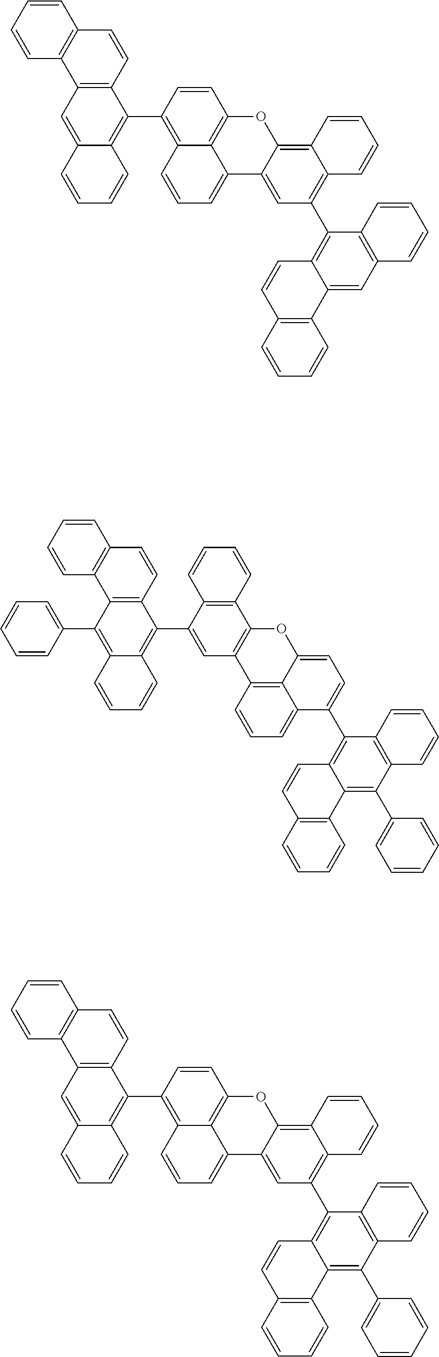 [SMILES: c1ccc2c(-c3cc4c(c5ccccc35)Oc3ccc(-c5c6ccccc6cc6c5ccc5ccccc56)c5cccc-4c35)c3ccc4ccccc4c3cc2c1 ; c1ccc(-c2c3ccccc3c(-c3cc4c(c5ccccc35)Oc3ccc(-c5c6ccccc6c(-c6ccccc6)c6c5ccc5ccccc56)c5cccc-4c35)c3ccc4ccccc4c23)cc1 ; c1ccc(-c2c3ccccc3c(-c3cc4c(c5ccccc35)Oc3ccc(-c5c6ccccc6cc6c5ccc5ccccc56)c5cccc-4c35)c3ccc4ccccc4c23)cc1]

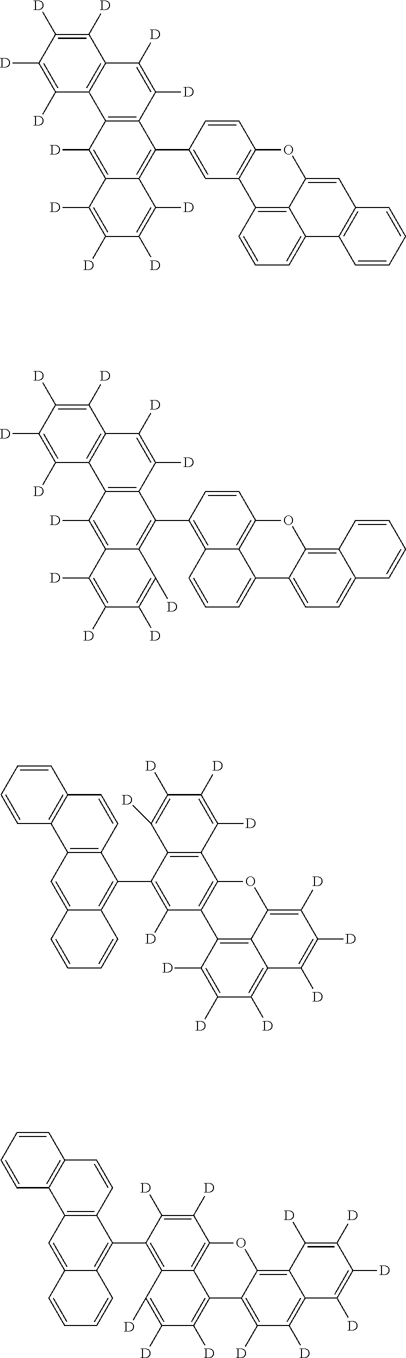

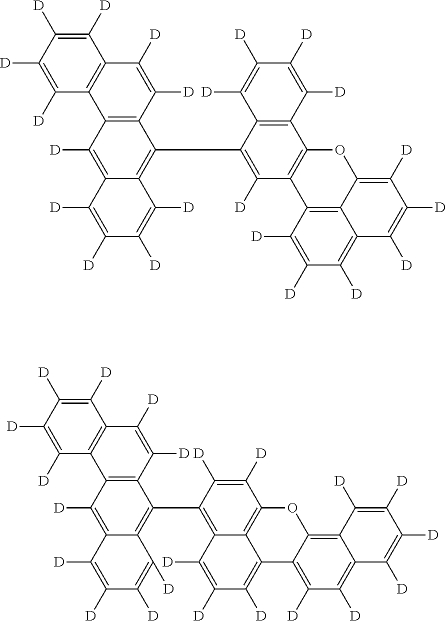 [SMILES: [2H]c1c([2H])c2c3c(c([2H])c([2H])c([2H])c3c1[2H])-c1c([2H])c(-c3c4c([2H])c([2H])c([2H])c([2H])c4c([2H])c4c3c([2H])c([2H])c3c([2H])c([2H])c([2H])c([2H])c34)c3c([2H])c([2H])c([2H])c([2H])c3c1O2 ; [2H]c1c([2H])c([2H])c2c3c(c([2H])c([2H])c2c1[2H])-c1c([2H])c([2H])c([2H])c2c(-c4c5c([2H])c([2H])c([2H])c([2H])c5c([2H])c5c4c([2H])c([2H])c4c([2H])c([2H])c([2H])c([2H])c45)c([2H])c([2H])c(c12)O3]

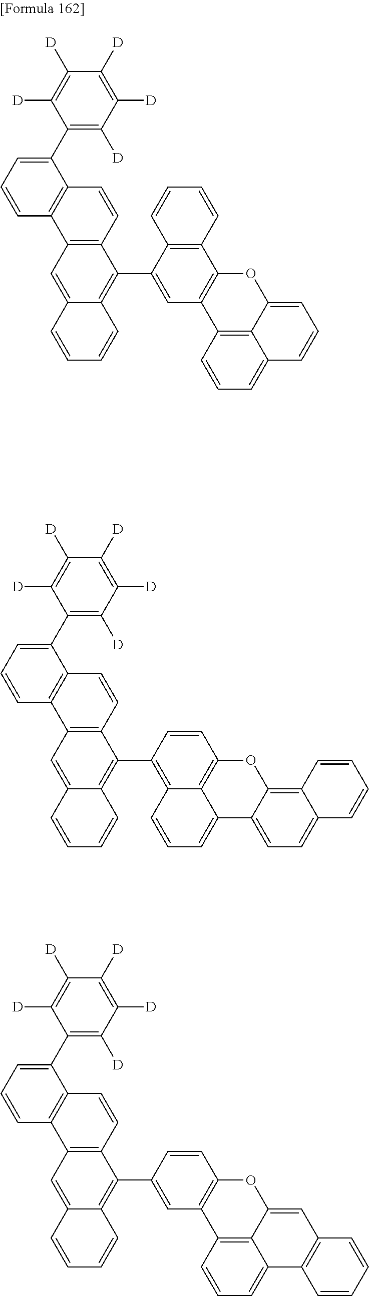 [SMILES: [2H]c1c([2H])c([2H])c(-c2cccc3c2ccc2c(-c4cc5c(c6ccccc46)Oc4cccc6cccc-5c46)c4ccccc4cc23)c([2H])c1[2H] ; [2H]c1c([2H])c([2H])c(-c2cccc3c2ccc2c(-c4ccc5c6c(cccc46)-c4ccc6ccccc6c4O5)c4ccccc4cc23)c([2H])c1[2H] ; [2H]c1c([2H])c([2H])c(-c2cccc3c2ccc2c(-c4ccc5c(c4)-c4cccc6c4c(cc4ccccc46)O5)c4ccccc4cc23)c([2H])c1[2H]]

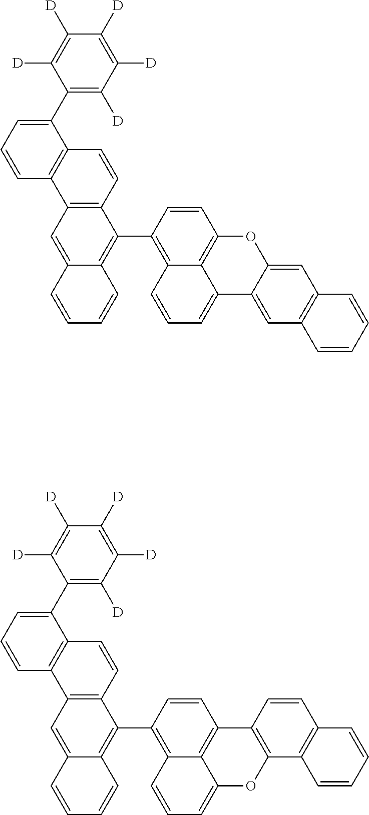 [SMILES: [2H]c1c([2H])c([2H])c(-c2cccc3c2ccc2c(-c4ccc5c6c(cccc46)-c4cc6ccccc6cc4O5)c4ccccc4cc23)c([2H])c1[2H] ; [2H]c1c([2H])c([2H])c(-c2cccc3c2ccc2c(-c4ccc5c6c(cccc46)Oc4c-5ccc5ccccc45)c4ccccc4cc23)c([2H])c1[2H]]

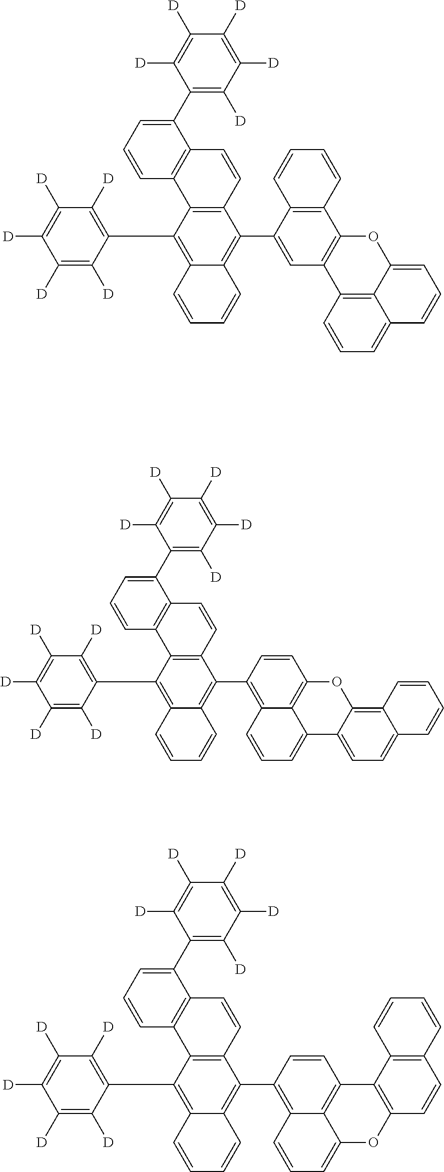 [SMILES: [2H]c1c([2H])c([2H])c(-c2cccc3c2ccc2c(-c4cc5c(c6ccccc46)Oc4cccc6cccc-5c46)c4ccccc4c(-c4c([2H])c([2H])c([2H])c([2H])c4[2H])c23)c([2H])c1[2H] ; [2H]c1c([2H])c([2H])c(-c2cccc3c2ccc2c(-c4ccc5c6c(cccc46)-c4ccc6ccccc6c4O5)c4ccccc4c(-c4c([2H])c([2H])c([2H])c([2H])c4[2H])c23)c([2H])c1[2H] ; [2H]c1c([2H])c([2H])c(-c2cccc3c2ccc2c(-c4ccc5c6c(cccc46)Oc4ccc6ccccc6c4-5)c4ccccc4c(-c4c([2H])c([2H])c([2H])c([2H])c4[2H])c23)c([2H])c1[2H]]

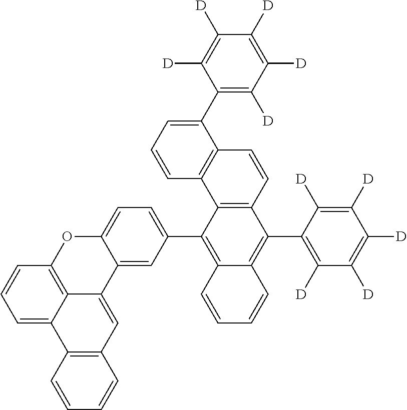 [SMILES: [2H]c1c([2H])c([2H])c(-c2cccc3c2ccc2c(-c4c([2H])c([2H])c([2H])c([2H])c4[2H])c4ccccc4c(-c4ccc5c(c4)-c4cc6ccccc6c6cccc(c46)O5)c23)c([2H])c1[2H]]

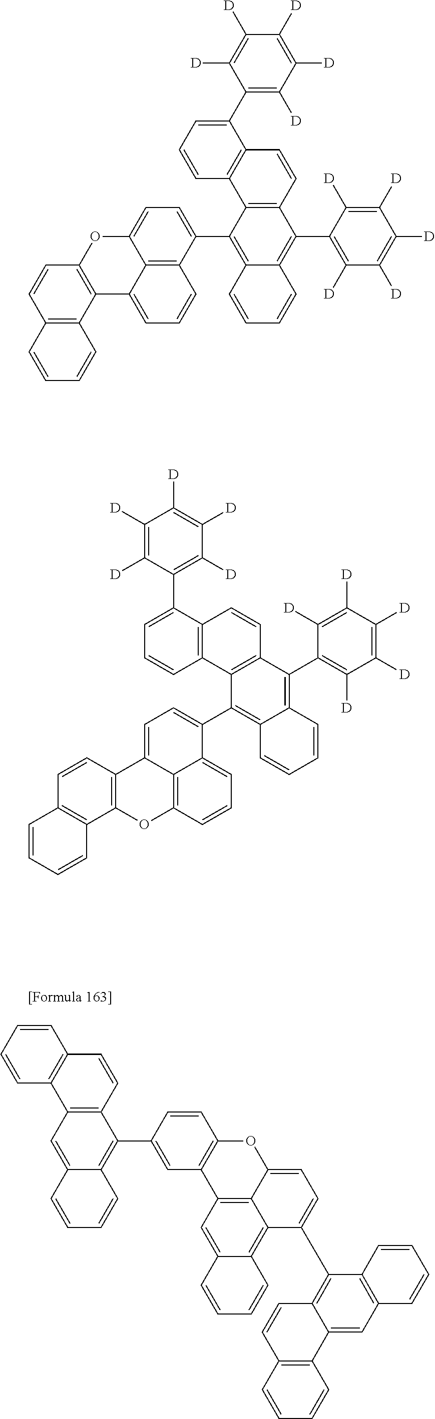 [SMILES: [2H]c1c([2H])c([2H])c(-c2cccc3c2ccc2c(-c4c([2H])c([2H])c([2H])c([2H])c4[2H])c4ccccc4c(-c4ccc5c6c(cccc46)-c4c(ccc6ccccc46)O5)c23)c([2H])c1[2H] ; [2H]c1c([2H])c([2H])c(-c2cccc3c2ccc2c(-c4c([2H])c([2H])c([2H])c([2H])c4[2H])c4ccccc4c(-c4ccc5c6c(cccc46)Oc4c-5ccc5ccccc45)c23)c([2H])c1[2H] ; c1ccc2c(-c3ccc4c(c3)-c3cc5ccccc5c5c(-c6c7ccccc7cc7c6ccc6ccccc67)ccc(c35)O4)c3ccc4ccccc4c3cc2c1]

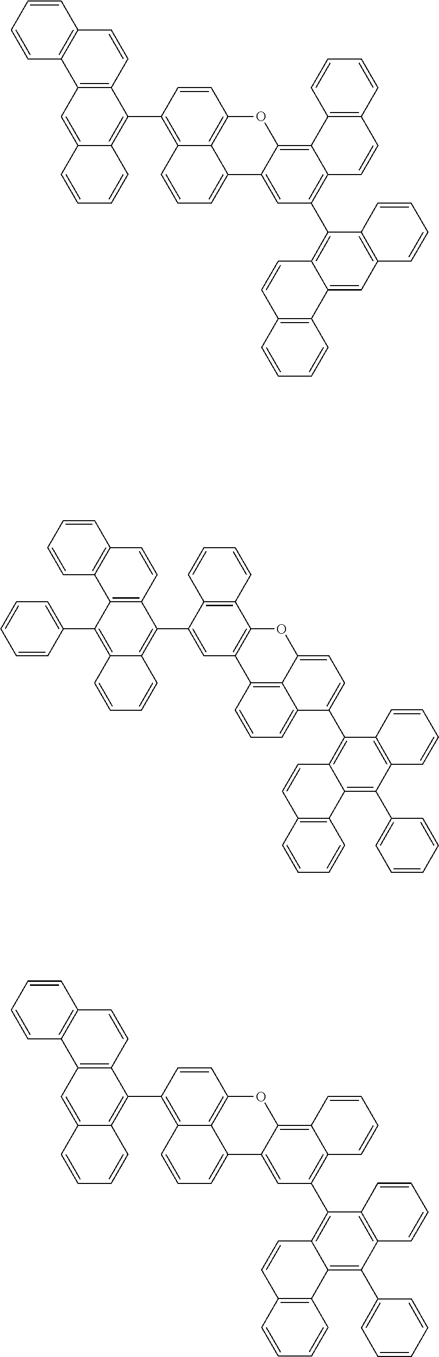 [SMILES: c1ccc2c(-c3ccc4c5c(cccc35)-c3cc(-c5c6ccccc6cc6c5ccc5ccccc56)c5ccc6ccccc6c5c3O4)c3ccc4ccccc4c3cc2c1 ; c1ccc(-c2c3ccccc3c(-c3cc4c(c5ccccc35)Oc3ccc(-c5c6ccccc6c(-c6ccccc6)c6c5ccc5ccccc56)c5cccc-4c35)c3ccc4ccccc4c23)cc1 ; c1ccc(-c2c3ccccc3c(-c3cc4c(c5ccccc35)Oc3ccc(-c5c6ccccc6cc6c5ccc5ccccc56)c5cccc-4c35)c3ccc4ccccc4c23)cc1]

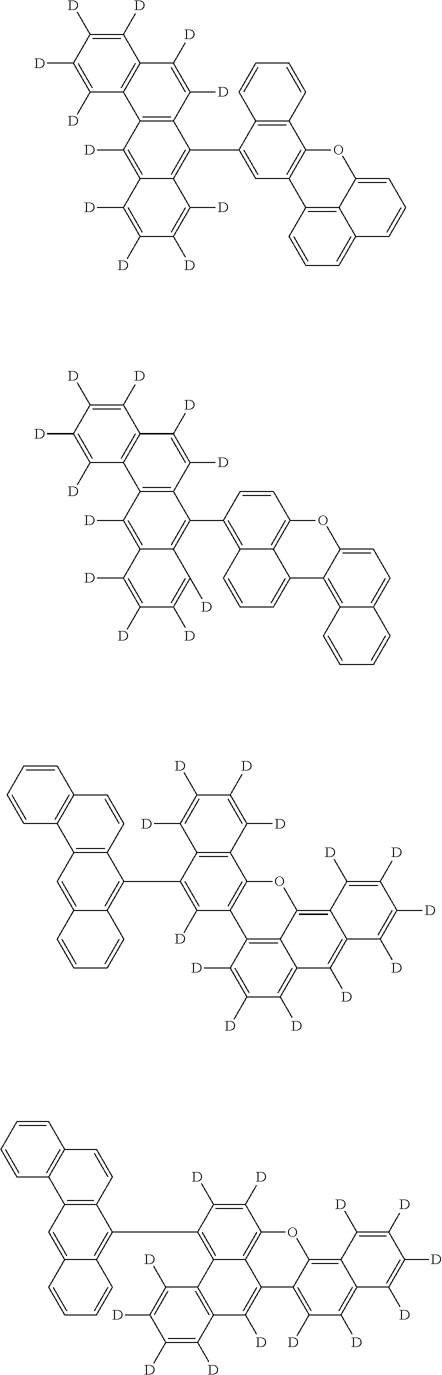

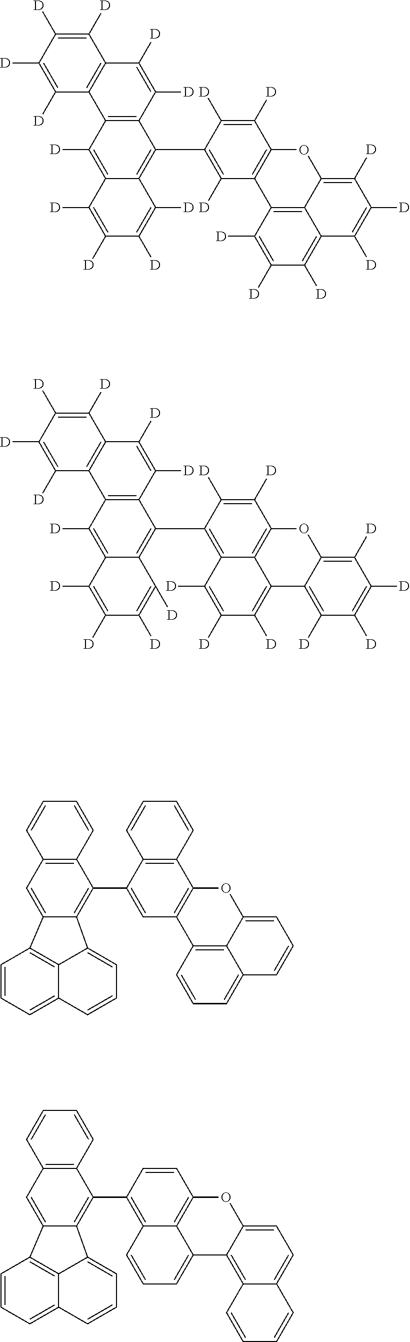 [SMILES: [2H]c1c([2H])c(-c2c3c([2H])c([2H])c([2H])c([2H])c3c([2H])c3c2c([2H])c([2H])c2c([2H])c([2H])c([2H])c([2H])c23)c([2H])c2c1Oc1c([2H])c([2H])c([2H])c3c([2H])c([2H])c([2H])c-2c13 ; [2H]c1c([2H])c([2H])c2c(c1[2H])Oc1c([2H])c([2H])c(-c3c4c([2H])c([2H])c([2H])c([2H])c4c([2H])c4c3c([2H])c([2H])c3c([2H])c([2H])c([2H])c([2H])c34)c3c([2H])c([2H])c([2H])c-2c13 ; c1ccc2c(-c3cc4c(c5ccccc35)Oc3cccc5cccc-4c35)c3c(cc2c1)-c1cccc2cccc-3c12 ; c1ccc2c3c(ccc2c1)Oc1ccc(-c2c4c(cc5ccccc25)-c2cccc5cccc-4c25)c2cccc-3c12]

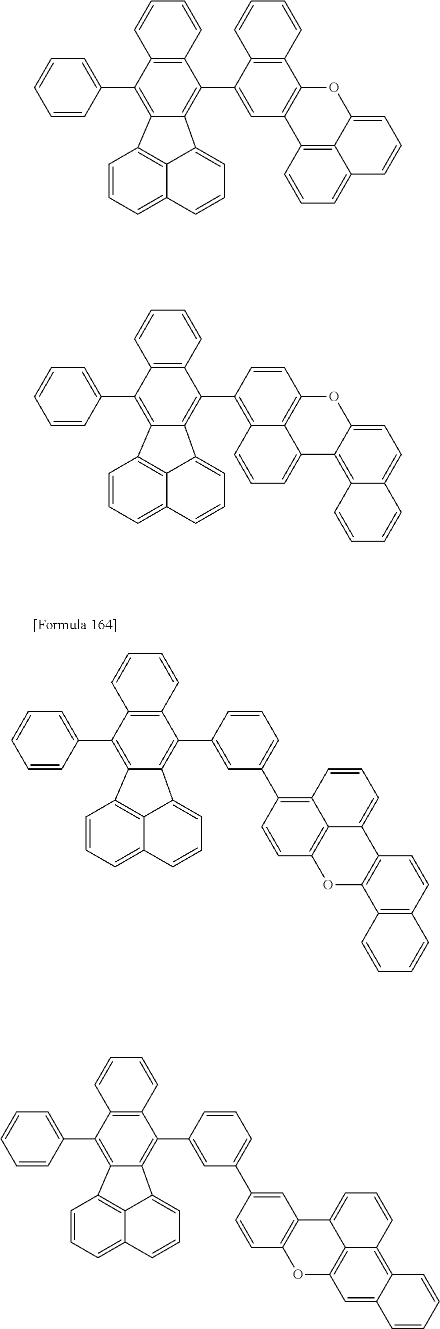 [SMILES: c1ccc(-c2c3c(c(-c4cc5c(c6ccccc46)Oc4cccc6cccc-5c46)c4ccccc24)-c2cccc4cccc-3c24)cc1 ; c1ccc(-c2c3c(c(-c4ccc5c6c(cccc46)-c4c(ccc6ccccc46)O5)c4ccccc24)-c2cccc4cccc-3c24)cc1 ; c1ccc(-c2c3c(c(-c4cccc(-c5ccc6c7c(cccc57)-c5ccc7ccccc7c5O6)c4)c4ccccc24)-c2cccc4cccc-3c24)cc1 ; c1ccc(-c2c3c(c(-c4cccc(-c5ccc6c(c5)-c5cccc7c5c(cc5ccccc57)O6)c4)c4ccccc24)-c2cccc4cccc-3c24)cc1]

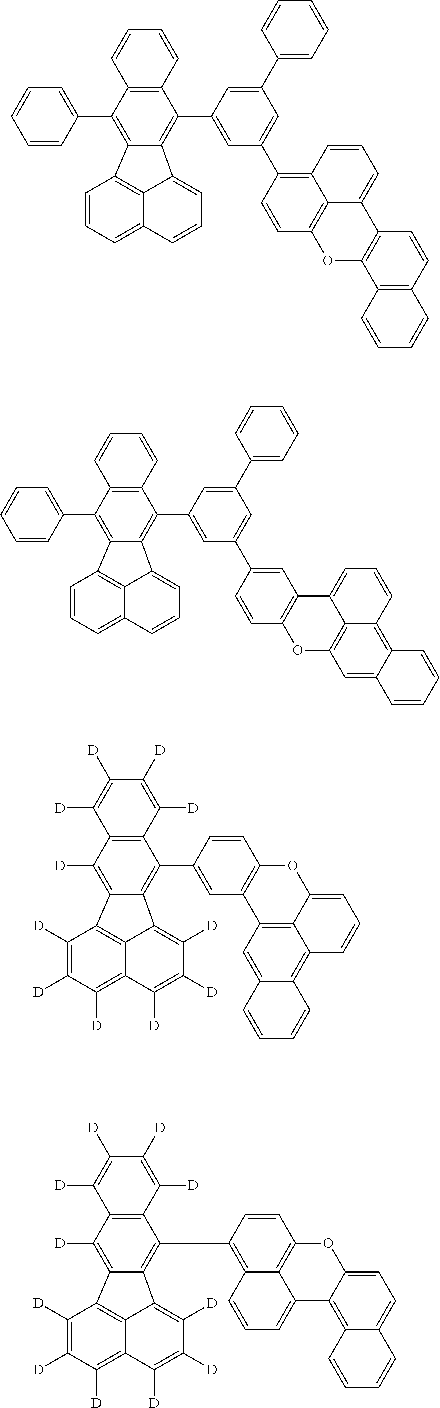 [SMILES: c1ccc(-c2cc(-c3c4c(c(-c5ccccc5)c5ccccc35)-c3cccc5cccc-4c35)cc(-c3ccc4c5c(cccc35)-c3ccc5ccccc5c3O4)c2)cc1 ; c1ccc(-c2cc(-c3ccc4c(c3)-c3cccc5c3c(cc3ccccc35)O4)cc(-c3c4c(c(-c5ccccc5)c5ccccc35)-c3cccc5cccc-4c35)c2)cc1 ; [2H]c1c([2H])c([2H])c2c(-c3ccc4c(c3)-c3cc5ccccc5c5cccc(c35)O4)c3c(c([2H])c2c1[2H])-c1c([2H])c([2H])c([2H])c2c([2H])c([2H])c([2H])c-3c12 ; [2H]c1c([2H])c([2H])c2c(-c3ccc4c5c(cccc35)-c3c(ccc5ccccc35)O4)c3c(c([2H])c2c1[2H])-c1c([2H])c([2H])c([2H])c2c([2H])c([2H])c([2H])c-3c12]

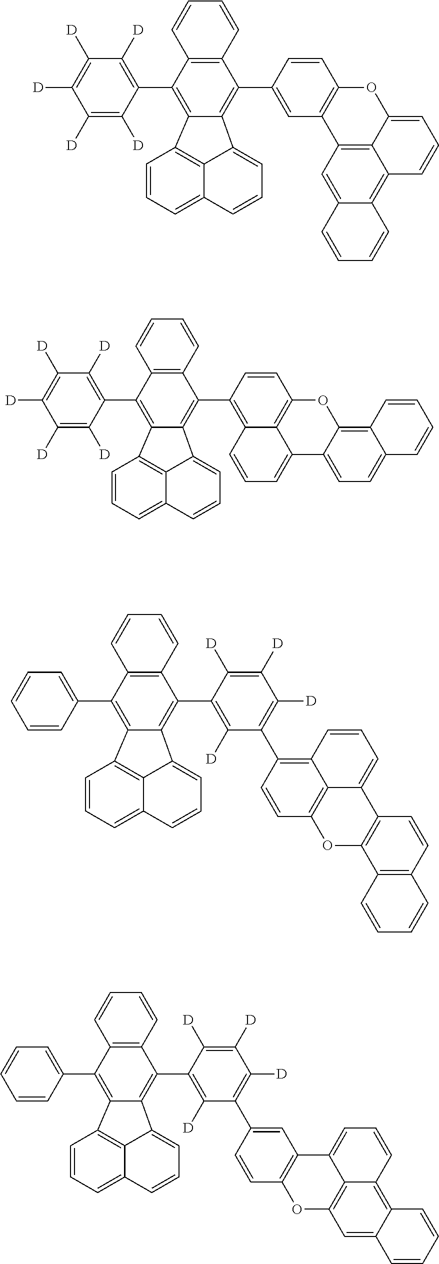 [SMILES: [2H]c1c([2H])c([2H])c(-c2c3c(c(-c4ccc5c(c4)-c4cc6ccccc6c6cccc(c46)O5)c4ccccc24)-c2cccc4cccc-3c24)c([2H])c1[2H] ; [2H]c1c([2H])c([2H])c(-c2c3c(c(-c4ccc5c6c(cccc46)-c4ccc6ccccc6c4O5)c4ccccc24)-c2cccc4cccc-3c24)c([2H])c1[2H] ; [2H]c1c([2H])c(-c2c3c(c(-c4ccccc4)c4ccccc24)-c2cccc4cccc-3c24)c([2H])c(-c2ccc3c4c(cccc24)-c2ccc4ccccc4c2O3)c1[2H] ; [2H]c1c([2H])c(-c2ccc3c(c2)-c2cccc4c2c(cc2ccccc24)O3)c([2H])c(-c2c3c(c(-c4ccccc4)c4ccccc24)-c2cccc4cccc-3c24)c1[2H]]

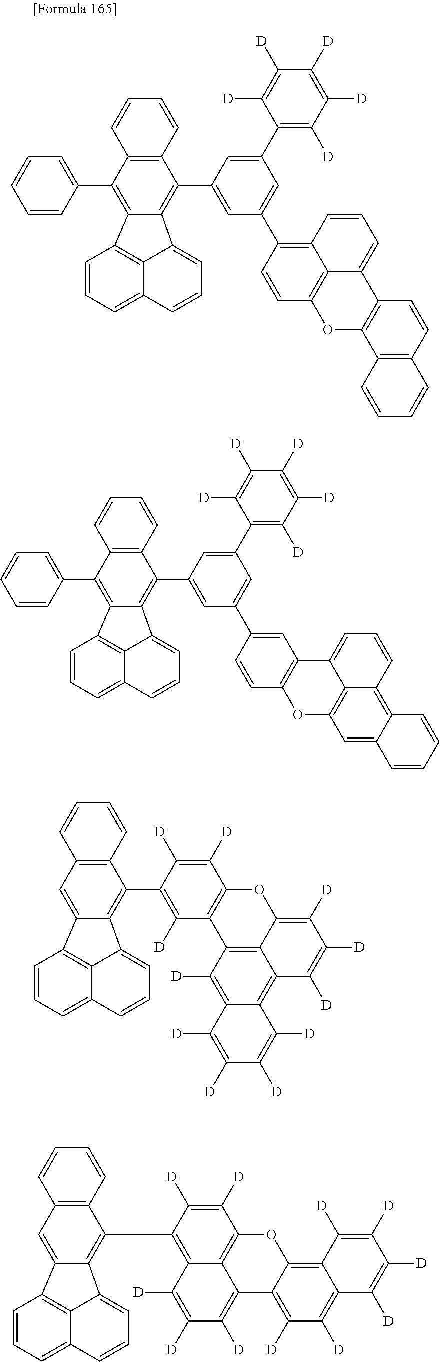

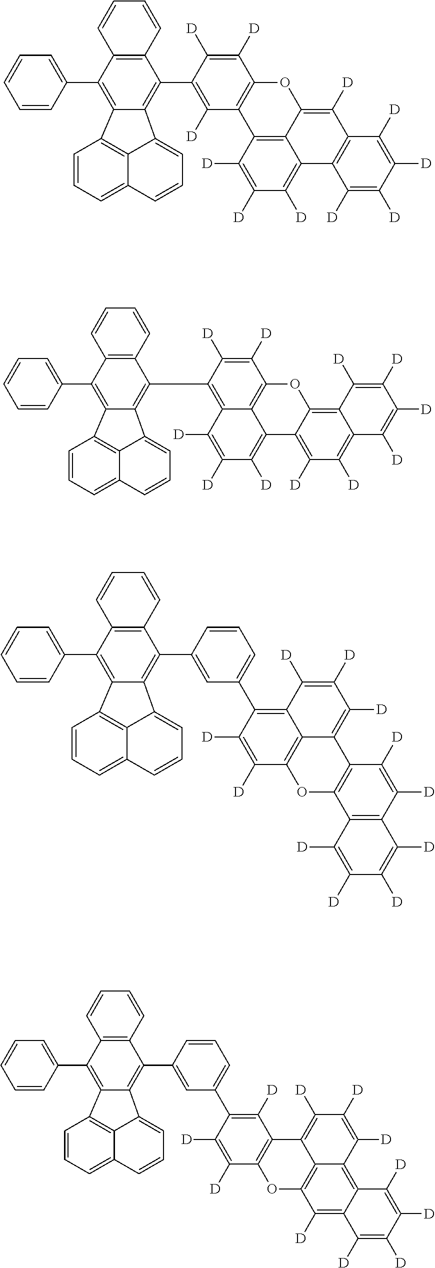

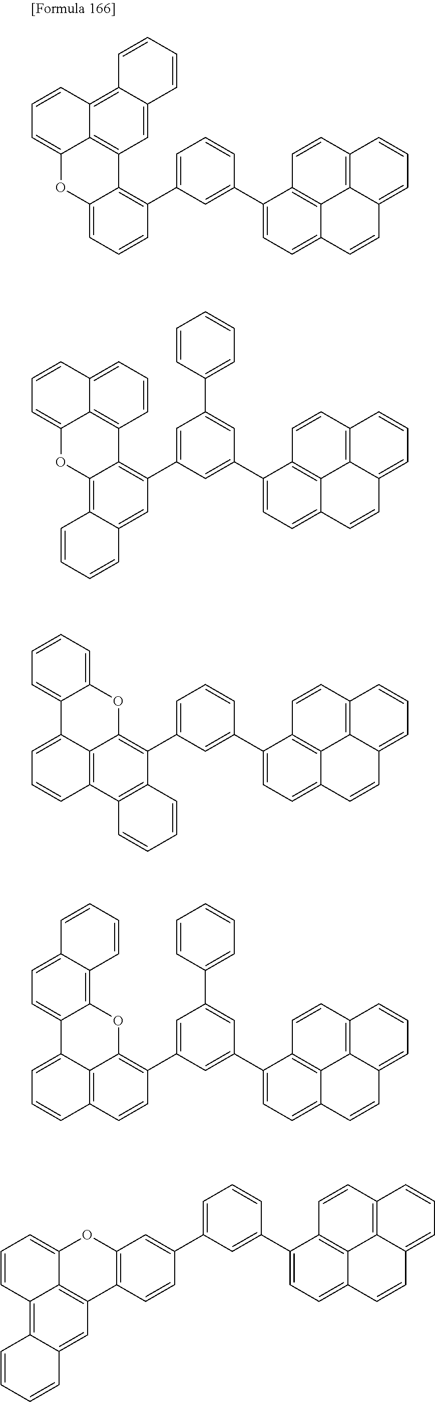 [SMILES: c1cc(-c2cccc3c2-c2cc4ccccc4c4cccc(c24)O3)cc(-c2ccc3ccc4cccc5ccc2c3c45)c1 ; c1ccc(-c2cc(-c3cc4ccccc4c4c3-c3cccc5cccc(c35)O4)cc(-c3ccc4ccc5cccc6ccc3c4c56)c2)cc1 ; c1cc(-c2c3c4c(cccc4c4ccccc24)-c2ccccc2O3)cc(-c2ccc3ccc4cccc5ccc2c3c45)c1 ; c1ccc(-c2cc(-c3ccc4cccc5c4c3Oc3c-5ccc4ccccc34)cc(-c3ccc4ccc5cccc6ccc3c4c56)c2)cc1 ; c1cc(-c2ccc3c(c2)Oc2cccc4c2c-3cc2ccccc24)cc(-c2ccc3ccc4cccc5ccc2c3c45)c1]

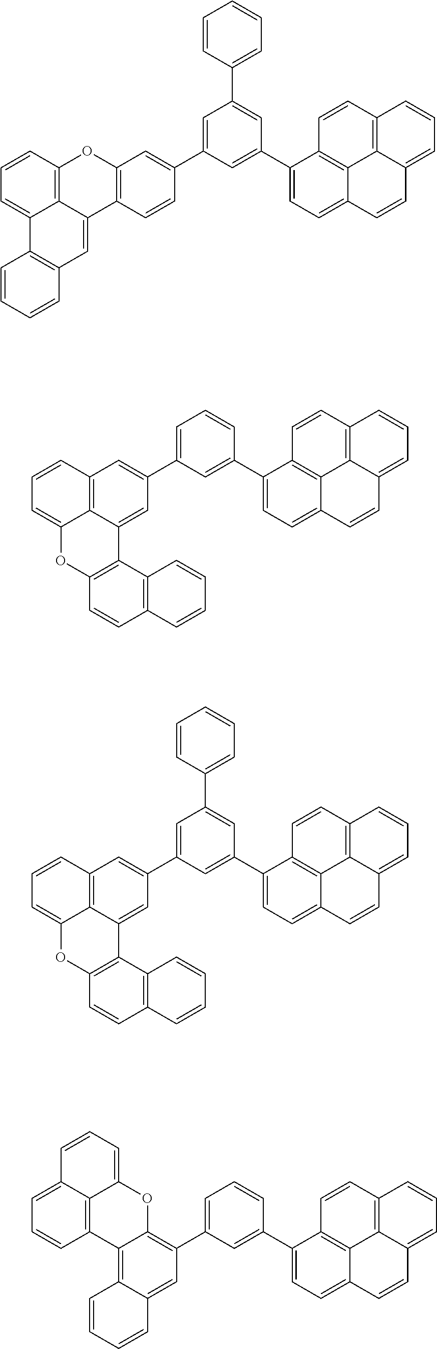 [SMILES: c1ccc(-c2cc(-c3ccc4c(c3)Oc3cccc5c3c-4cc3ccccc35)cc(-c3ccc4ccc5cccc6ccc3c4c56)c2)cc1 ; c1cc(-c2cc3c4c(cccc4c2)Oc2ccc4ccccc4c2-3)cc(-c2ccc3ccc4cccc5ccc2c3c45)c1 ; c1ccc(-c2cc(-c3cc4c5c(cccc5c3)Oc3ccc5ccccc5c3-4)cc(-c3ccc4ccc5cccc6ccc3c4c56)c2)cc1 ; c1cc(-c2cc3ccccc3c3c2Oc2cccc4cccc-3c24)cc(-c2ccc3ccc4cccc5ccc2c3c45)c1]

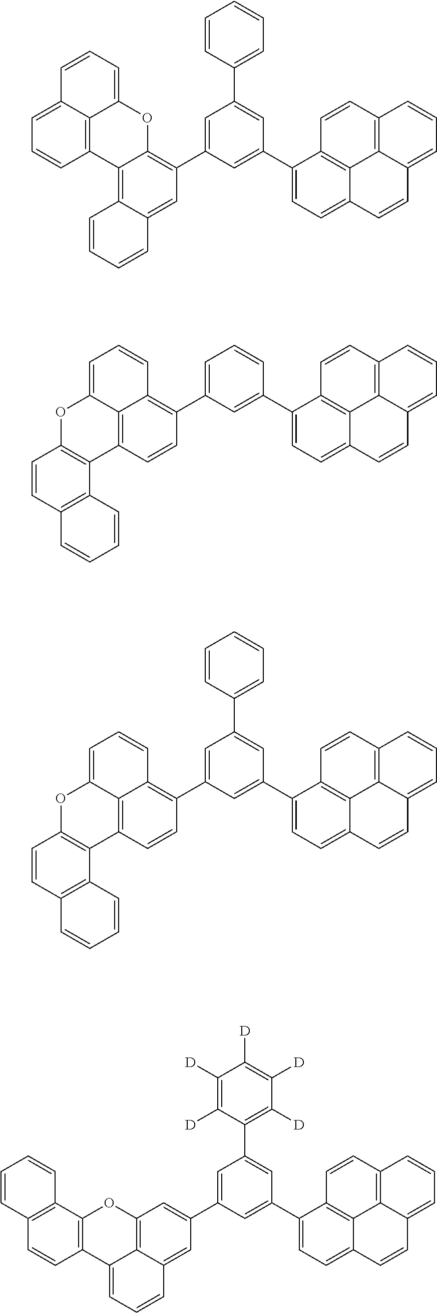 [SMILES: c1ccc(-c2cc(-c3cc4ccccc4c4c3Oc3cccc5cccc-4c35)cc(-c3ccc4ccc5cccc6ccc3c4c56)c2)cc1 ; c1cc(-c2ccc3c4c(cccc24)Oc2ccc4ccccc4c2-3)cc(-c2ccc3ccc4cccc5ccc2c3c45)c1 ; c1ccc(-c2cc(-c3ccc4c5c(cccc35)Oc3ccc5ccccc5c3-4)cc(-c3ccc4ccc5cccc6ccc3c4c56)c2)cc1 ; [2H]c1c([2H])c([2H])c(-c2cc(-c3cc4c5c(cccc5c3)-c3ccc5ccccc5c3O4)cc(-c3ccc4ccc5cccc6ccc3c4c56)c2)c([2H])c1[2H]]

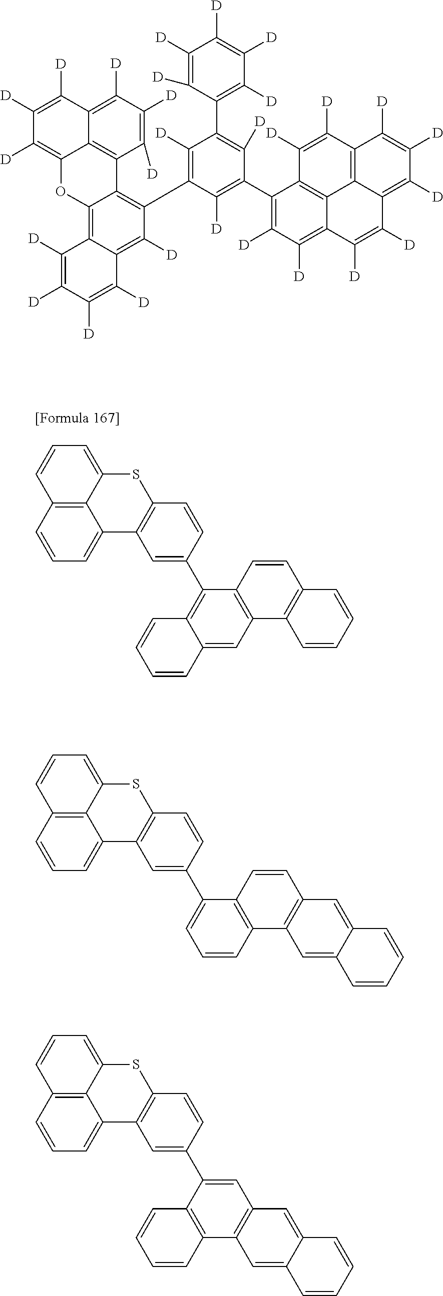 [SMILES: [2H]c1c([2H])c([2H])c(-c2c([2H])c(-c3c4c(c5c([2H])c([2H])c([2H])c([2H])c5c3[2H])Oc3c([2H])c([2H])c([2H])c5c([2H])c([2H])c([2H])c-4c35)c([2H])c(-c3c([2H])c([2H])c4c([2H])c([2H])c5c([2H])c([2H])c([2H])c6c([2H])c([2H])c3c4c56)c2[2H])c([2H])c1[2H] ; c1ccc2c(-c3ccc4c(c3)-c3cccc5cccc(c35)S4)c3ccc4ccccc4c3cc2c1 ; c1ccc2cc3c(ccc4c(-c5ccc6c(c5)-c5cccc7cccc(c57)S6)cccc43)cc2c1 ; c1ccc2cc3c(cc(-c4ccc5c(c4)-c4cccc6cccc(c46)S5)c4ccccc43)cc2c1]

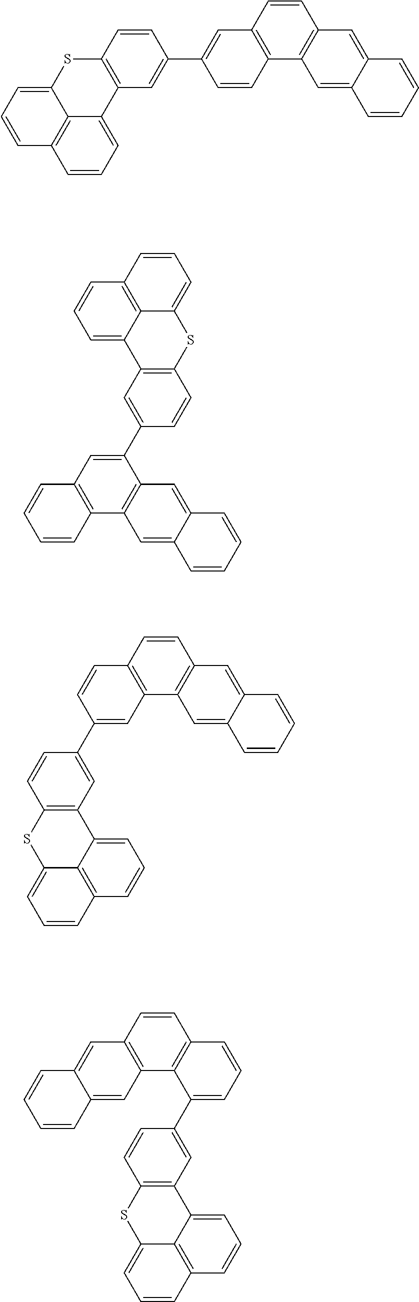 [SMILES: c1ccc2cc3c(ccc4cc(-c5ccc6c(c5)-c5cccc7cccc(c57)S6)ccc43)cc2c1 ; c1ccc2cc3c(cc2c1)c(-c1ccc2c(c1)-c1cccc4cccc(c14)S2)cc1ccccc13 ; c1ccc2cc3c(ccc4ccc(-c5ccc6c(c5)-c5cccc7cccc(c57)S6)cc43)cc2c1 ; c1ccc2cc3c(ccc4cccc(-c5ccc6c(c5)-c5cccc7cccc(c57)S6)c43)cc2c1]

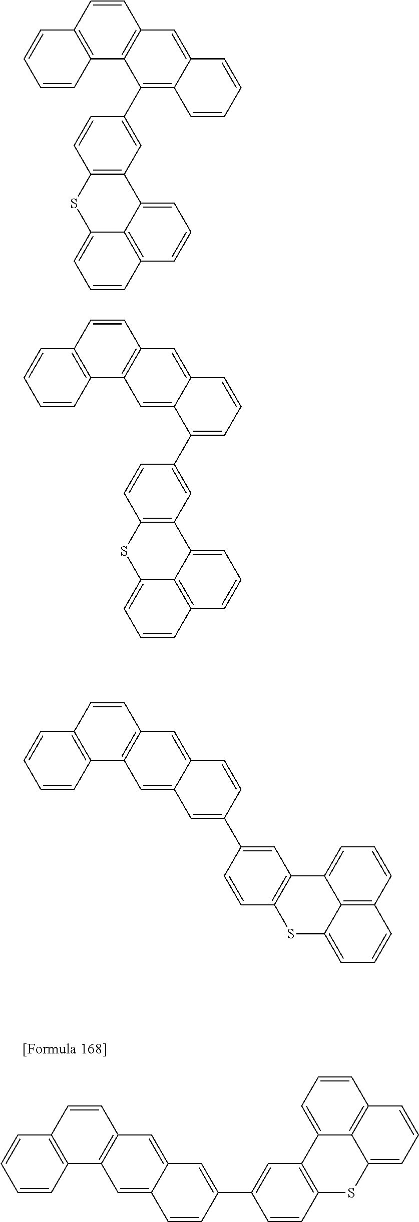

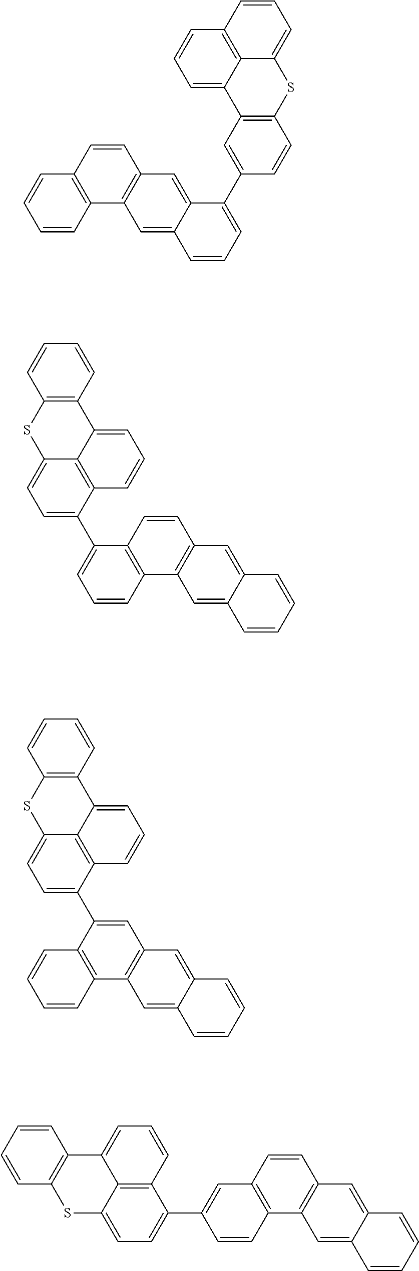 [SMILES: c1cc(-c2ccc3c(c2)-c2cccc4cccc(c24)S3)c2cc3ccc4ccccc4c3cc2c1 ; c1ccc2c(c1)Sc1ccc(-c3cccc4c3ccc3cc5ccccc5cc34)c3cccc-2c13 ; c1ccc2c(c1)Sc1ccc(-c3cc4cc5ccccc5cc4c4ccccc34)c3cccc-2c13 ; c1ccc2c(c1)Sc1ccc(-c3ccc4c(ccc5cc6ccccc6cc54)c3)c3cccc-2c13]

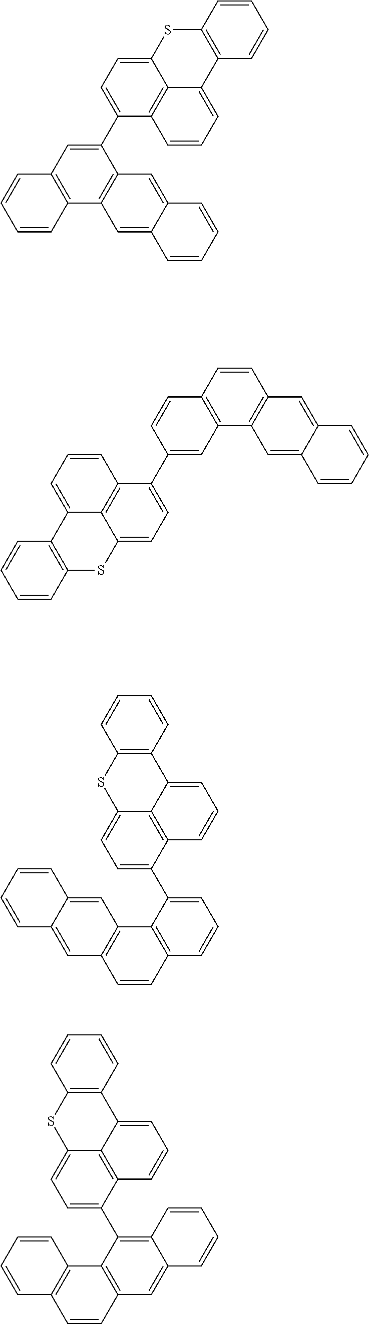 [SMILES: c1ccc2c(c1)Sc1ccc(-c3cc4ccccc4c4cc5ccccc5cc34)c3cccc-2c13 ; c1ccc2c(c1)Sc1ccc(-c3ccc4ccc5cc6ccccc6cc5c4c3)c3cccc-2c13 ; c1ccc2c(c1)Sc1ccc(-c3cccc4ccc5cc6ccccc6cc5c34)c3cccc-2c13 ; c1ccc2c(c1)Sc1ccc(-c3c4ccccc4cc4ccc5ccccc5c34)c3cccc-2c13]

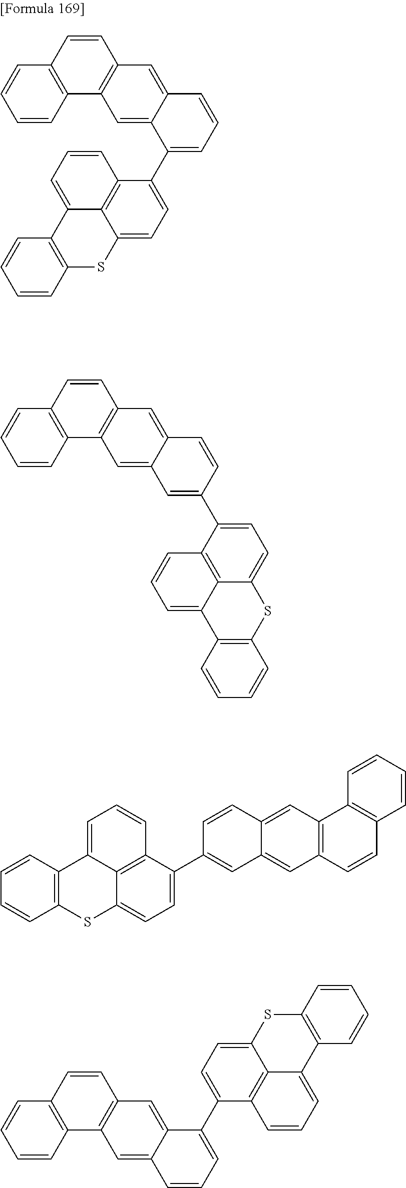 [SMILES: c1ccc2c(c1)Sc1ccc(-c3cccc4cc5ccc6ccccc6c5cc34)c3cccc-2c13 ; c1ccc2c(c1)Sc1ccc(-c3ccc4cc5ccc6ccccc6c5cc4c3)c3cccc-2c13 ; c1ccc2c(c1)Sc1ccc(-c3ccc4cc5c(ccc6ccccc65)cc4c3)c3cccc-2c13 ; c1ccc2c(c1)Sc1ccc(-c3cccc4cc5c(ccc6ccccc65)cc34)c3cccc-2c13]

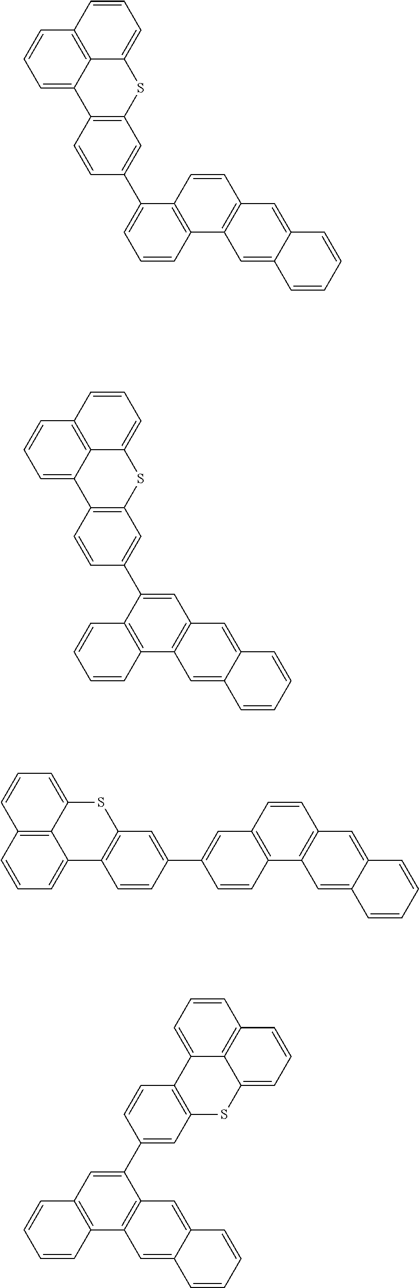 [SMILES: c1ccc2cc3c(ccc4c(-c5ccc6c(c5)Sc5cccc7cccc-6c57)cccc43)cc2c1 ; c1ccc2cc3c(cc(-c4ccc5c(c4)Sc4cccc6cccc-5c46)c4ccccc43)cc2c1 ; c1ccc2cc3c(ccc4cc(-c5ccc6c(c5)Sc5cccc7cccc-6c57)ccc43)cc2c1 ; c1ccc2cc3c(cc2c1)c(-c1ccc2c(c1)Sc1cccc4cccc-2c14)cc1ccccc13]

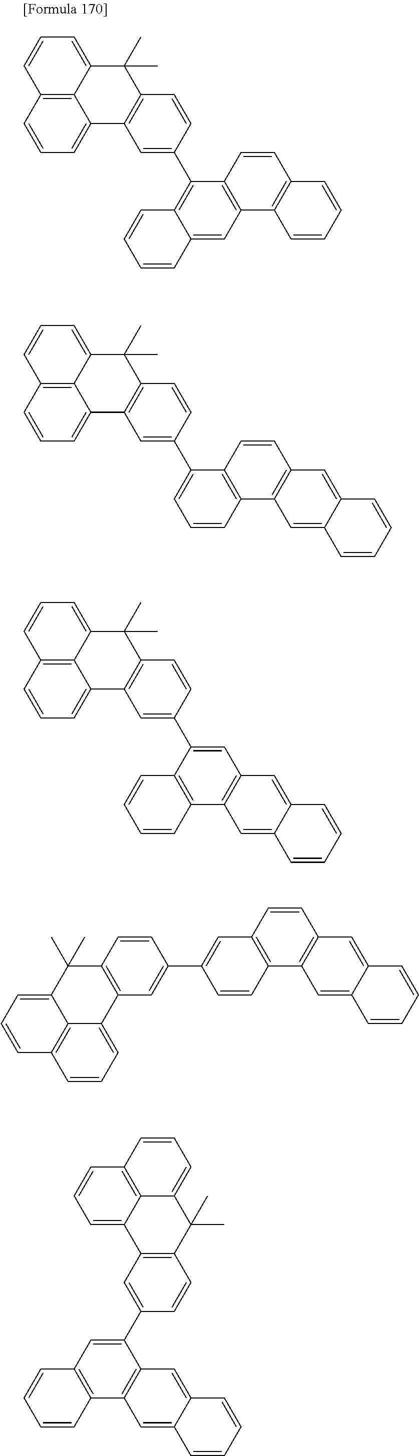

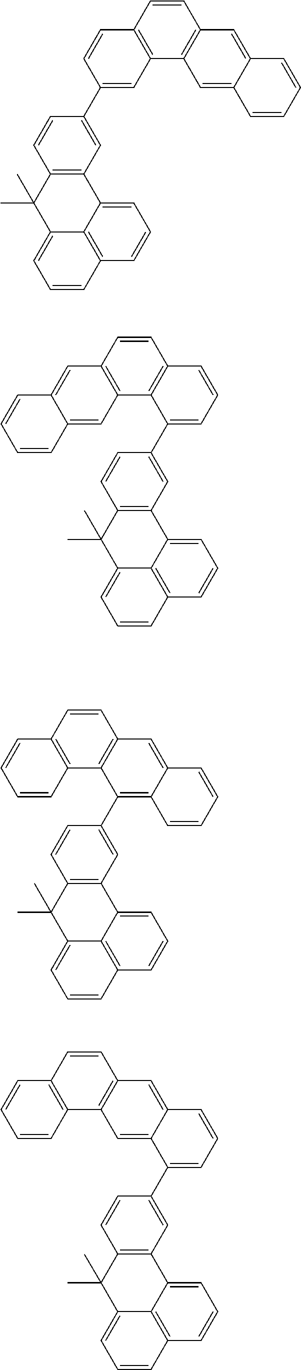

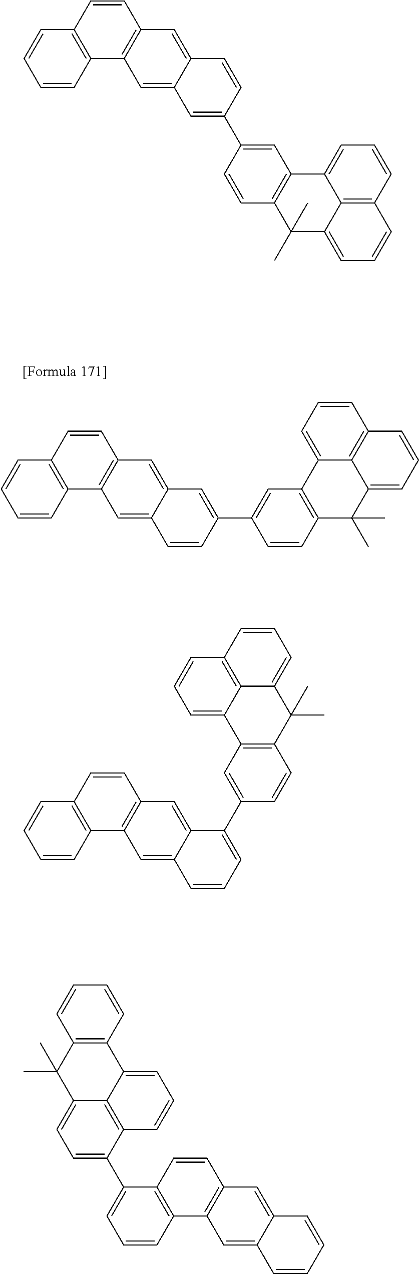

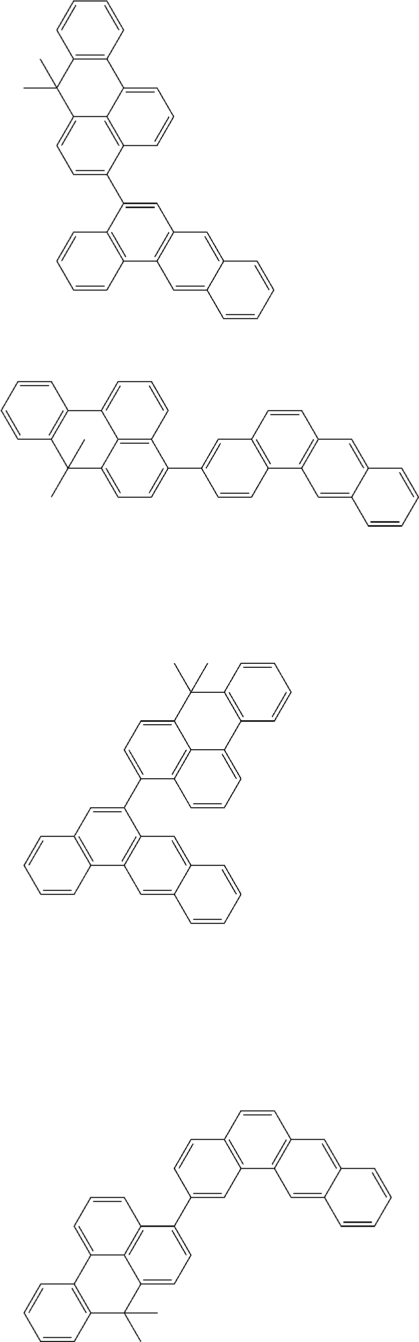

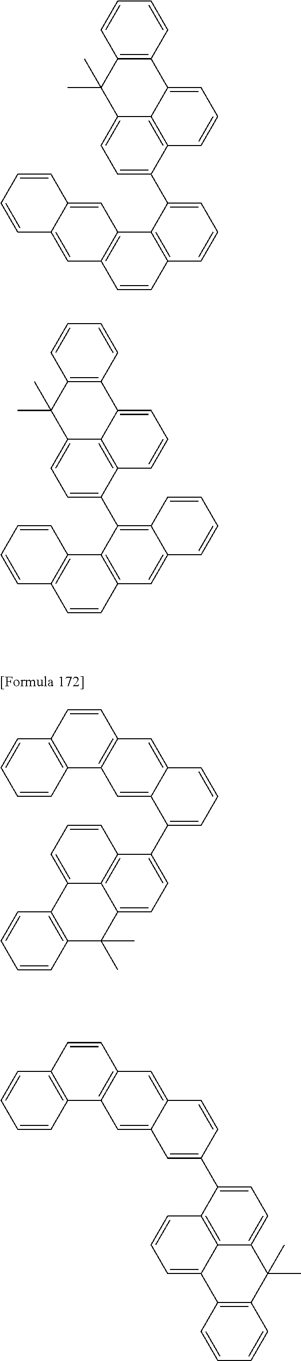

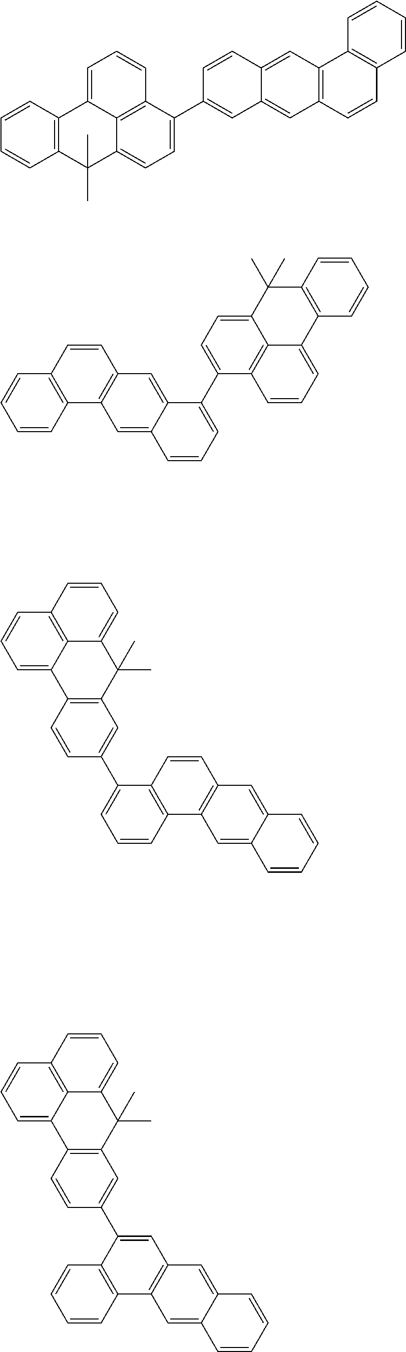

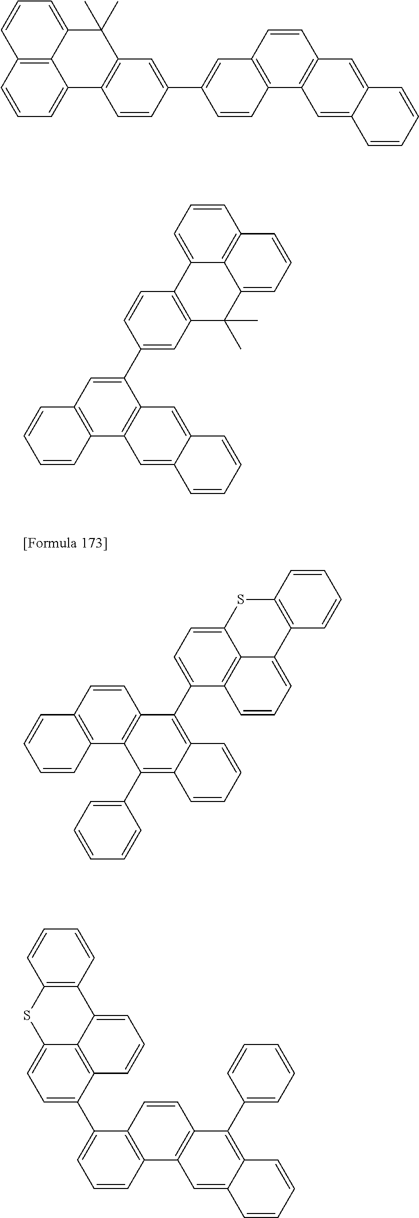

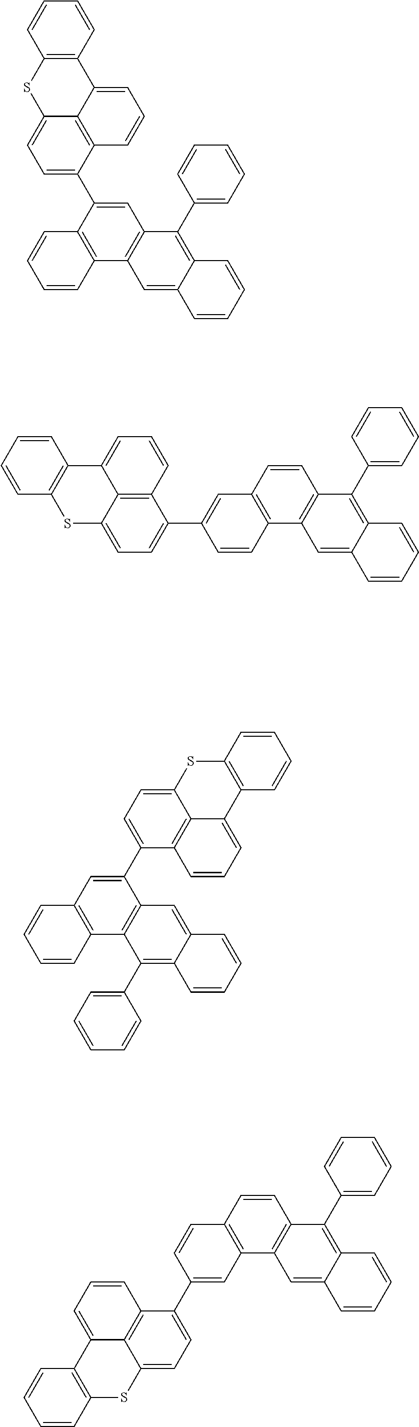 [SMILES: c1ccc(-c2c3ccccc3cc3c2cc(-c2ccc4c5c(cccc25)-c2ccccc2S4)c2ccccc23)cc1 ; c1ccc(-c2c3ccccc3cc3c2ccc2cc(-c4ccc5c6c(cccc46)-c4ccccc4S5)ccc23)cc1 ; c1ccc(-c2c3ccccc3cc3c(-c4ccc5c6c(cccc46)-c4ccccc4S5)cc4ccccc4c23)cc1 ; c1ccc(-c2c3ccccc3cc3c2ccc2ccc(-c4ccc5c6c(cccc46)-c4ccccc4S5)cc23)cc1]

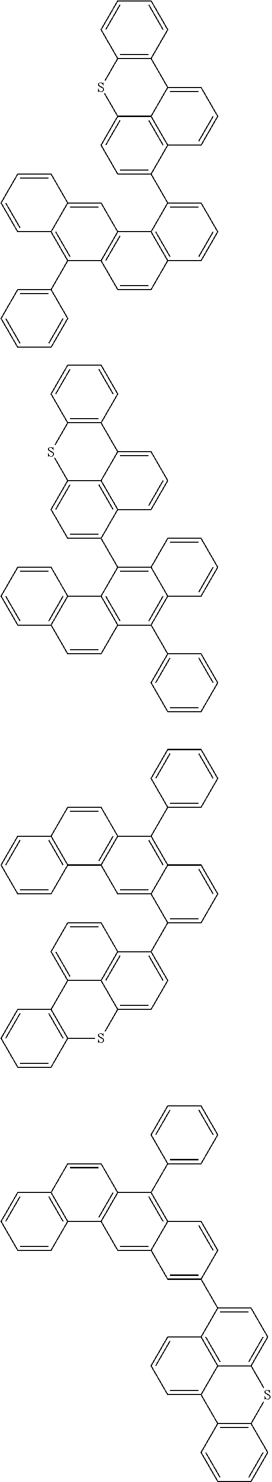

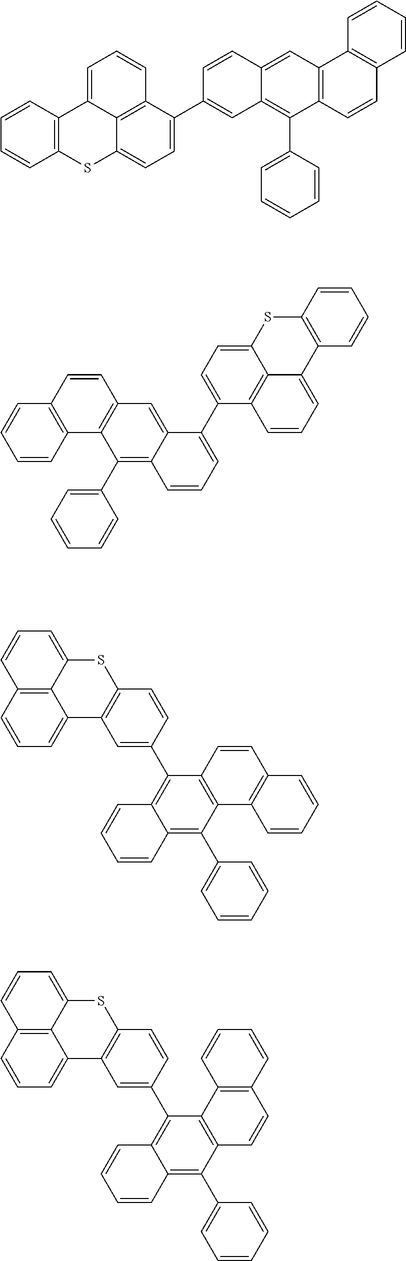 [SMILES: c1ccc(-c2c3cc(-c4ccc5c6c(cccc46)-c4ccccc4S5)ccc3cc3c2ccc2ccccc23)cc1 ; c1ccc(-c2c3cccc(-c4ccc5c6c(cccc46)-c4ccccc4S5)c3cc3ccc4ccccc4c23)cc1 ; c1ccc(-c2c3ccccc3c(-c3ccc4c(c3)-c3cccc5cccc(c35)S4)c3ccc4ccccc4c23)cc1 ; c1ccc(-c2c3ccccc3c(-c3ccc4c(c3)-c3cccc5cccc(c35)S4)c3c2ccc2ccccc23)cc1]

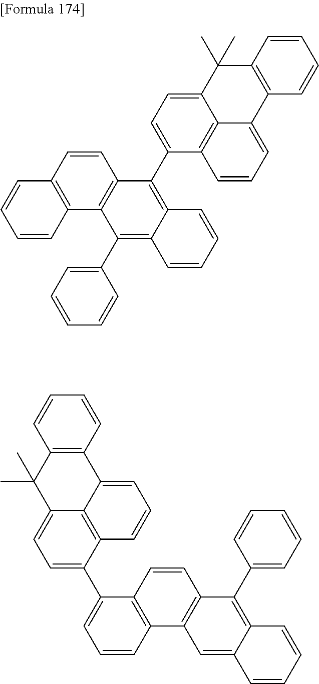

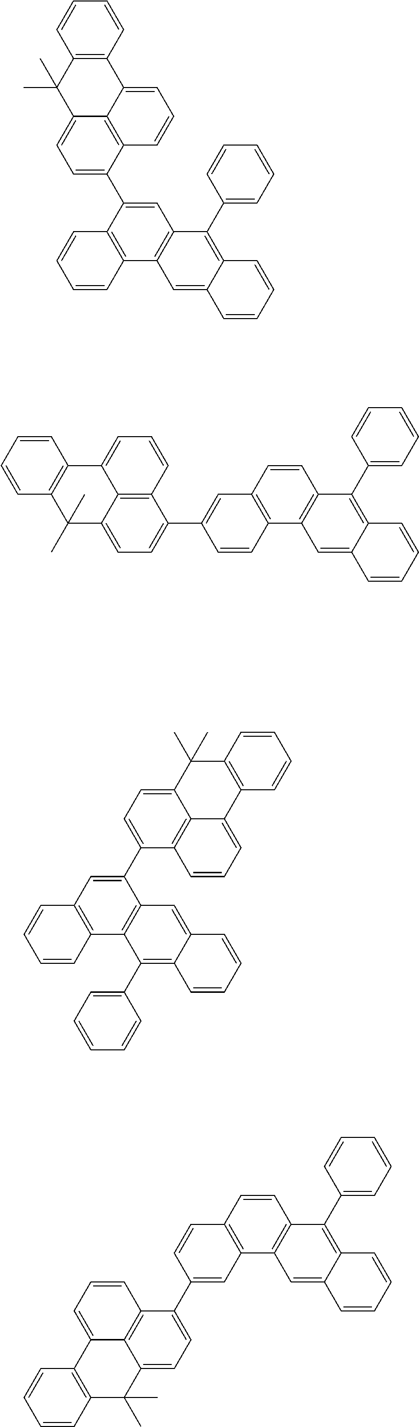

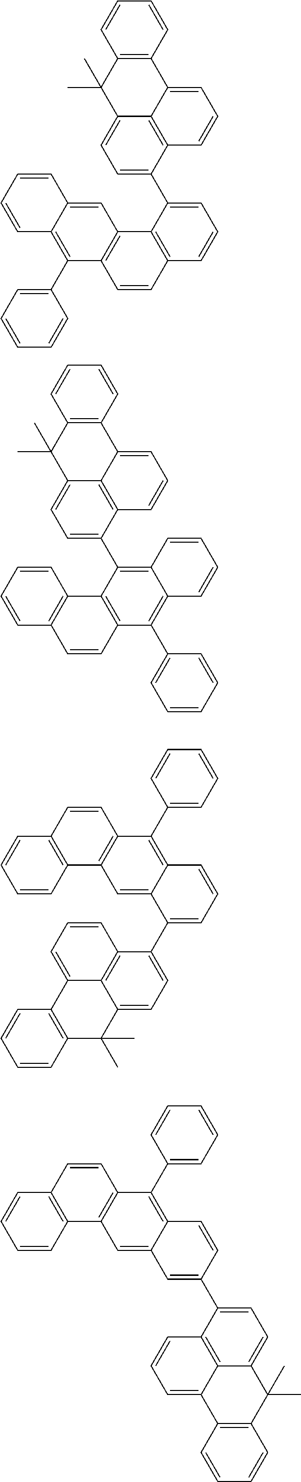

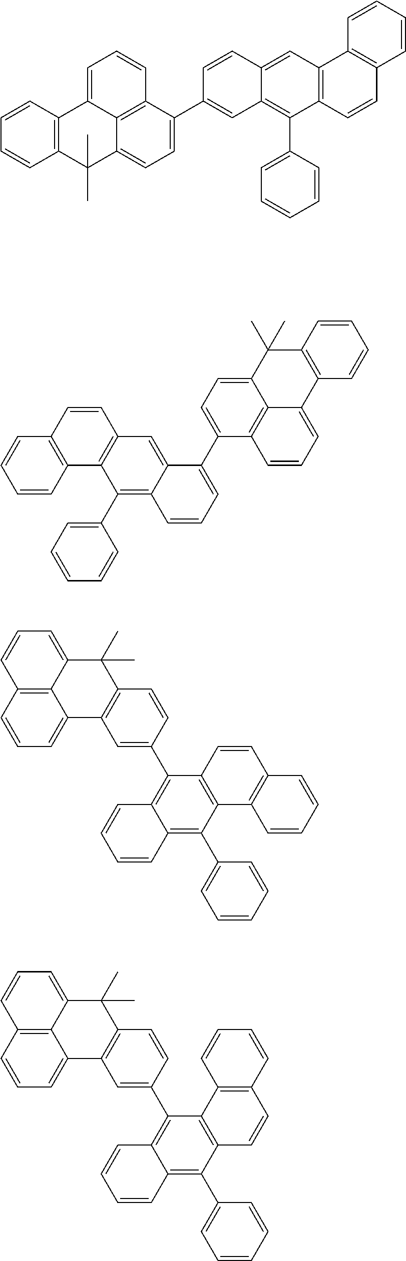

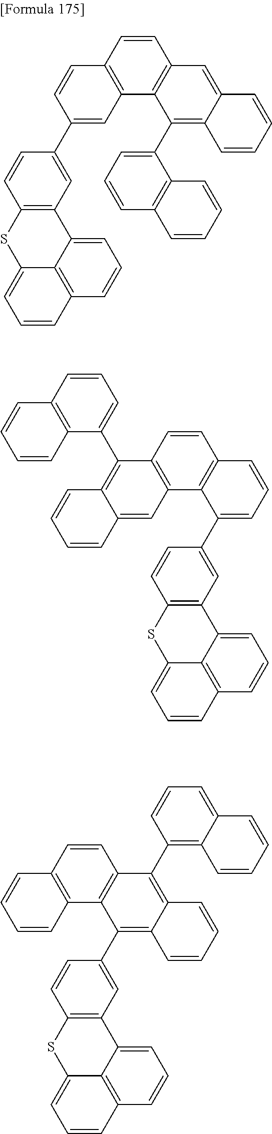 [SMILES: c1ccc2c(-c3c4ccccc4cc4ccc5ccc(-c6ccc7c(c6)-c6cccc8cccc(c68)S7)cc5c34)cccc2c1 ; c1ccc2c(-c3c4ccccc4cc4c3ccc3cccc(-c5ccc6c(c5)-c5cccc7cccc(c57)S6)c34)cccc2c1 ; c1ccc2c(-c3c4ccccc4c(-c4ccc5c(c4)-c4cccc6cccc(c46)S5)c4c3ccc3ccccc34)cccc2c1]

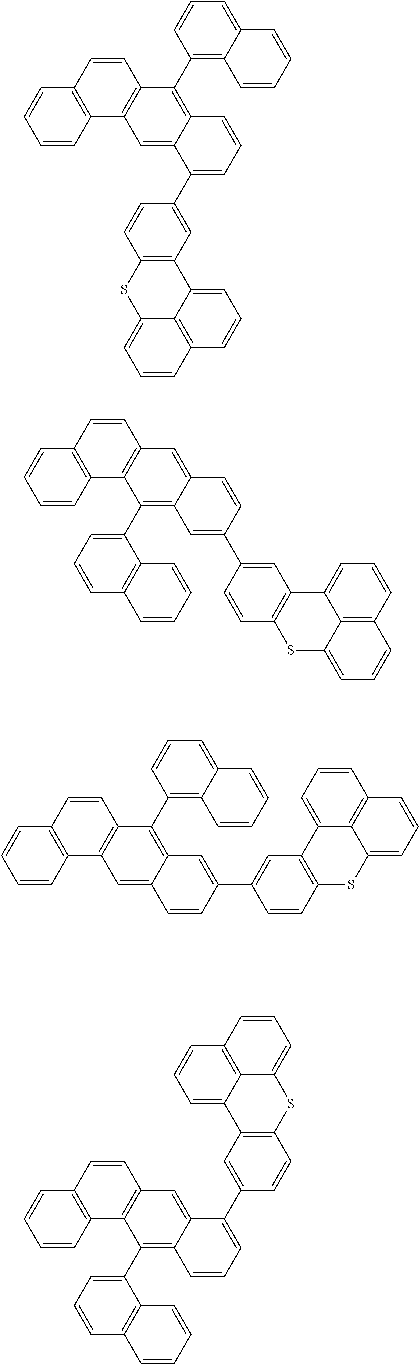

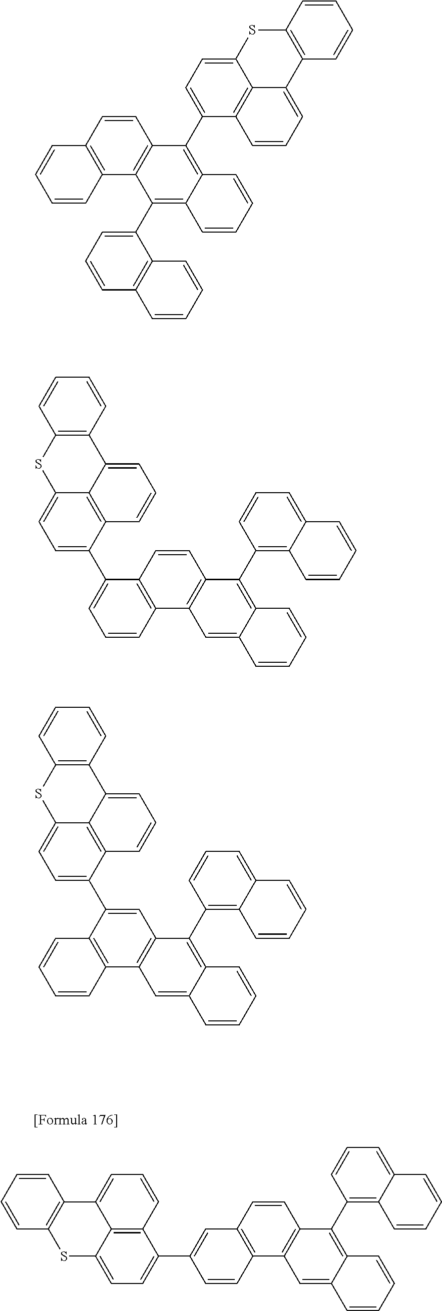 [SMILES: c1ccc2c(c1)Sc1ccc(-c3c4ccccc4c(-c4cccc5ccccc45)c4c3ccc3ccccc34)c3cccc-2c13 ; c1ccc2c(c1)Sc1ccc(-c3cccc4c3ccc3c(-c5cccc6ccccc56)c5ccccc5cc34)c3cccc-2c13 ; c1ccc2c(c1)Sc1ccc(-c3cc4c(-c5cccc6ccccc56)c5ccccc5cc4c4ccccc34)c3cccc-2c13 ; c1ccc2c(c1)Sc1ccc(-c3ccc4c(ccc5c(-c6cccc7ccccc67)c6ccccc6cc54)c3)c3cccc-2c13]

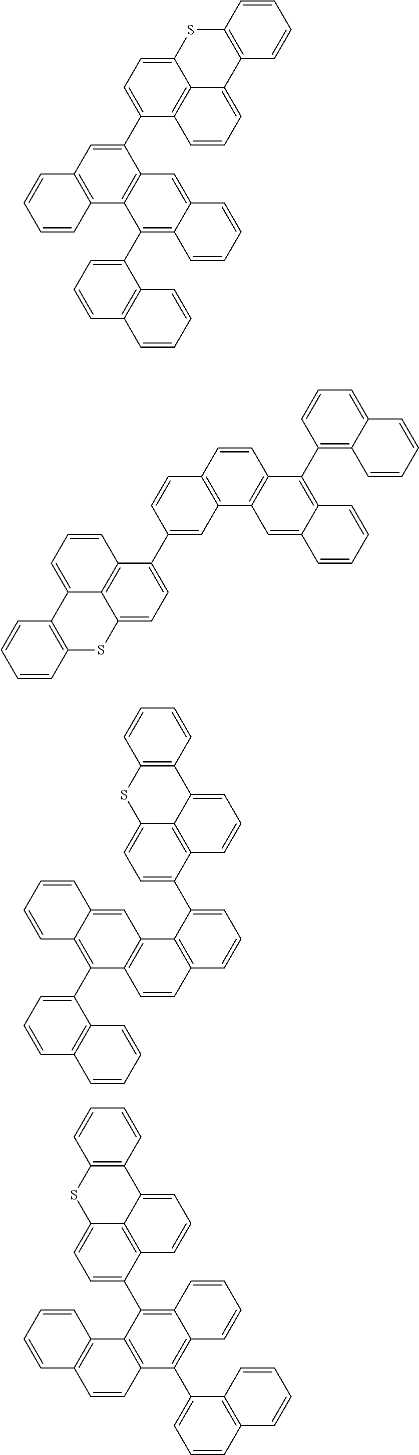

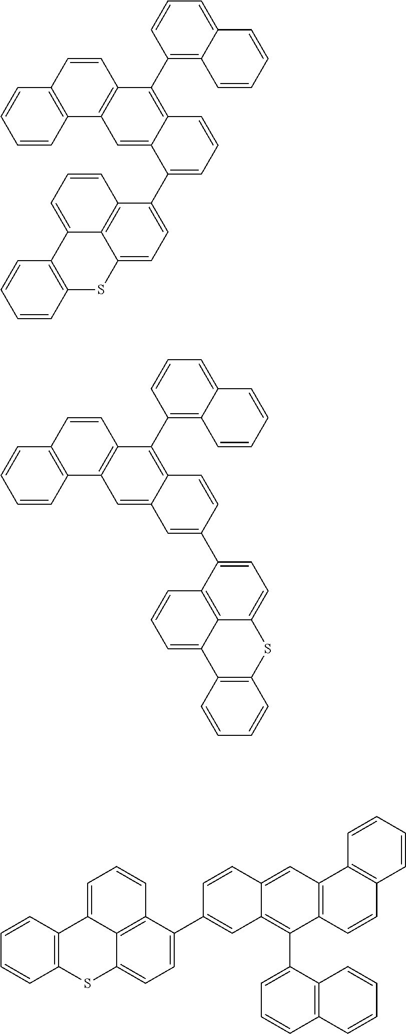 [SMILES: c1ccc2c(c1)Sc1ccc(-c3cccc4c(-c5cccc6ccccc56)c5ccc6ccccc6c5cc34)c3cccc-2c13 ; c1ccc2c(c1)Sc1ccc(-c3ccc4c(-c5cccc6ccccc56)c5ccc6ccccc6c5cc4c3)c3cccc-2c13 ; c1ccc2c(c1)Sc1ccc(-c3ccc4cc5c(ccc6ccccc65)c(-c5cccc6ccccc56)c4c3)c3cccc-2c13]

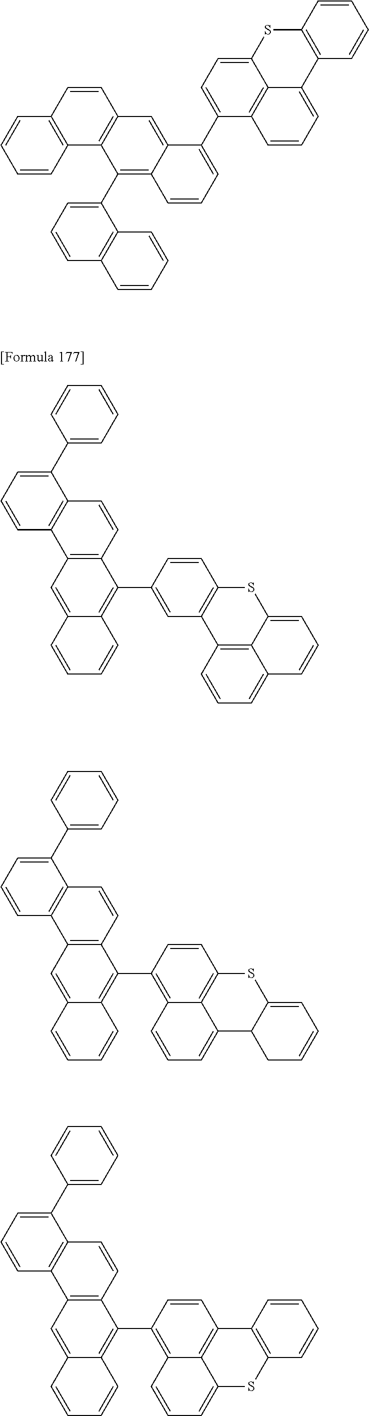 [SMILES: c1ccc2c(c1)Sc1ccc(-c3cccc4c(-c5cccc6ccccc56)c5c(ccc6ccccc65)cc34)c3cccc-2c13 ; c1ccc(-c2cccc3c2ccc2c(-c4ccc5c(c4)-c4cccc6cccc(c46)S5)c4ccccc4cc23)cc1 ; C1=CCC2C(=C1)Sc1ccc(-c3c4ccccc4cc4c3ccc3c(-c5ccccc5)cccc34)c3cccc2c13 ; c1ccc(-c2cccc3c2ccc2c(-c4ccc5c6c(cccc46)Sc4ccccc4-5)c4ccccc4cc23)cc1]

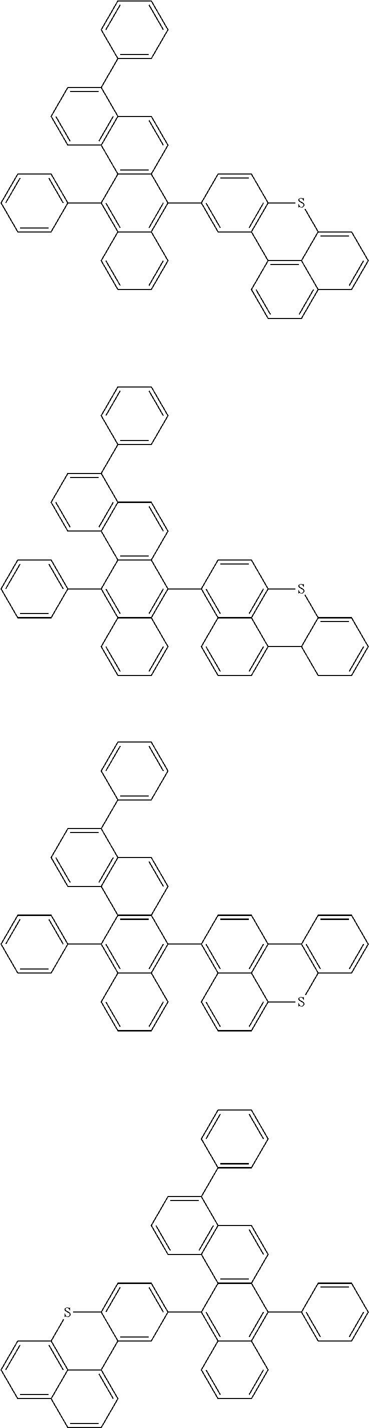 [SMILES: c1ccc(-c2cccc3c2ccc2c(-c4ccc5c(c4)-c4cccc6cccc(c46)S5)c4ccccc4c(-c4ccccc4)c23)cc1 ; C1=CCC2C(=C1)Sc1ccc(-c3c4ccccc4c(-c4ccccc4)c4c3ccc3c(-c5ccccc5)cccc34)c3cccc2c13 ; c1ccc(-c2cccc3c2ccc2c(-c4ccc5c6c(cccc46)Sc4ccccc4-5)c4ccccc4c(-c4ccccc4)c23)cc1 ; c1ccc(-c2cccc3c2ccc2c(-c4ccccc4)c4ccccc4c(-c4ccc5c(c4)-c4cccc6cccc(c46)S5)c23)cc1]

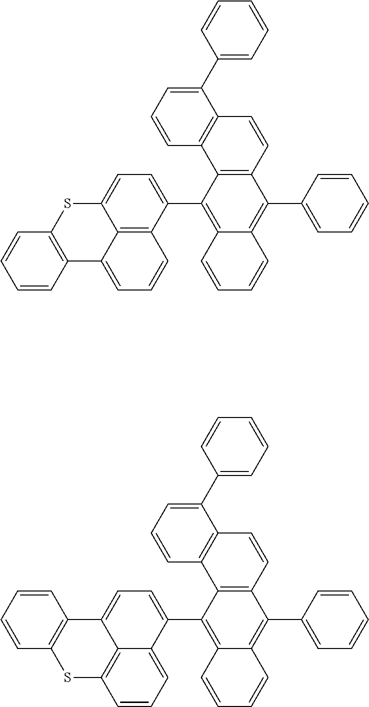 [SMILES: c1ccc(-c2cccc3c2ccc2c(-c4ccccc4)c4ccccc4c(-c4ccc5c6c(cccc46)-c4ccccc4S5)c23)cc1 ; c1ccc(-c2cccc3c2ccc2c(-c4ccccc4)c4ccccc4c(-c4ccc5c6c(cccc46)Sc4ccccc4-5)c23)cc1]

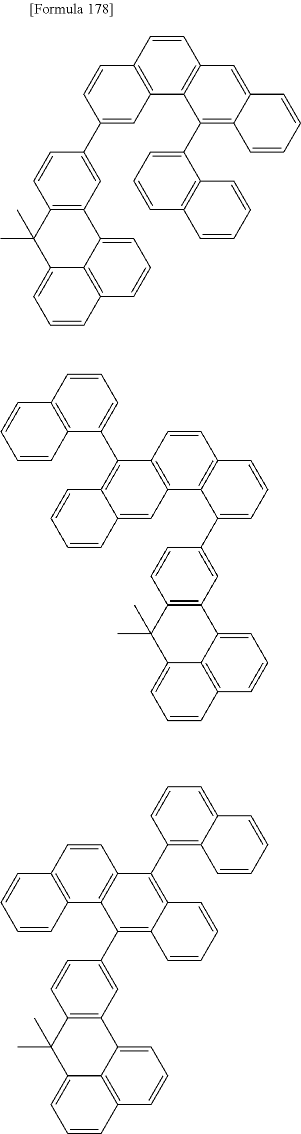

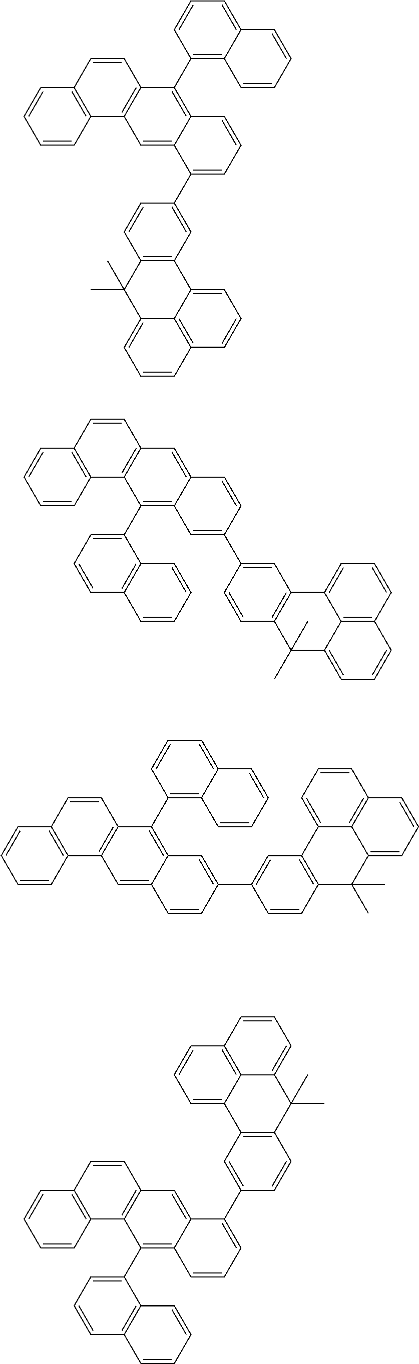

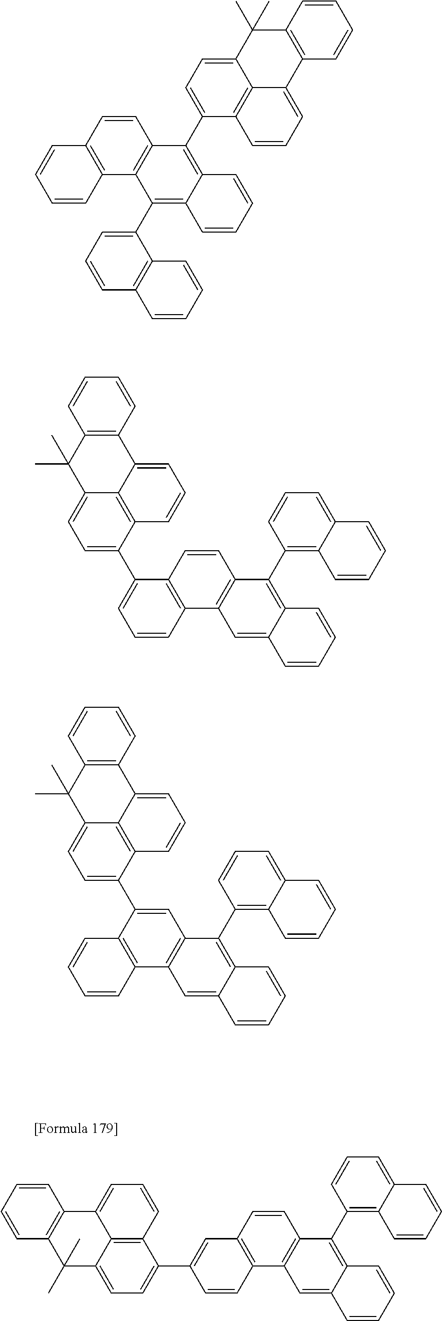

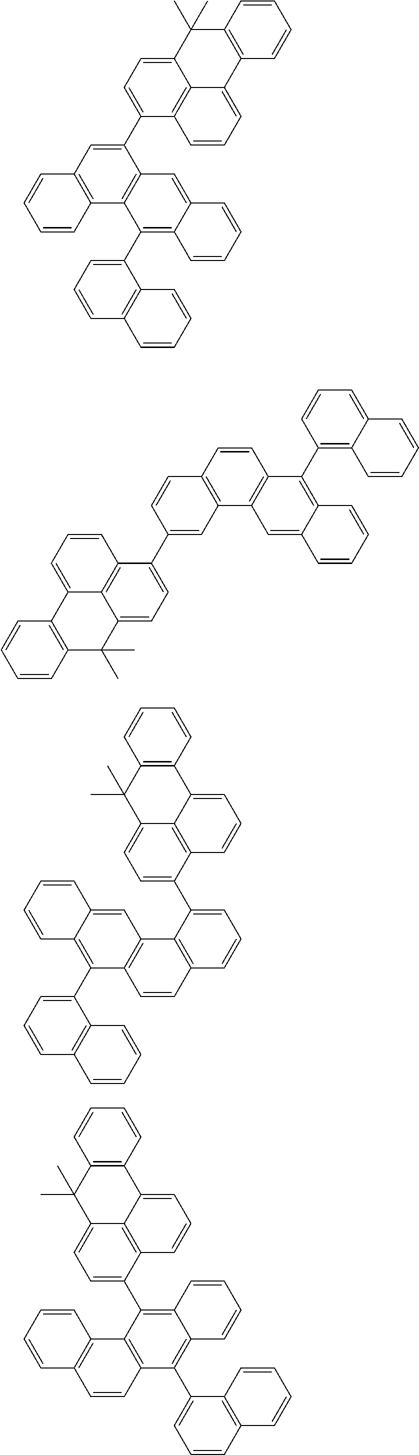

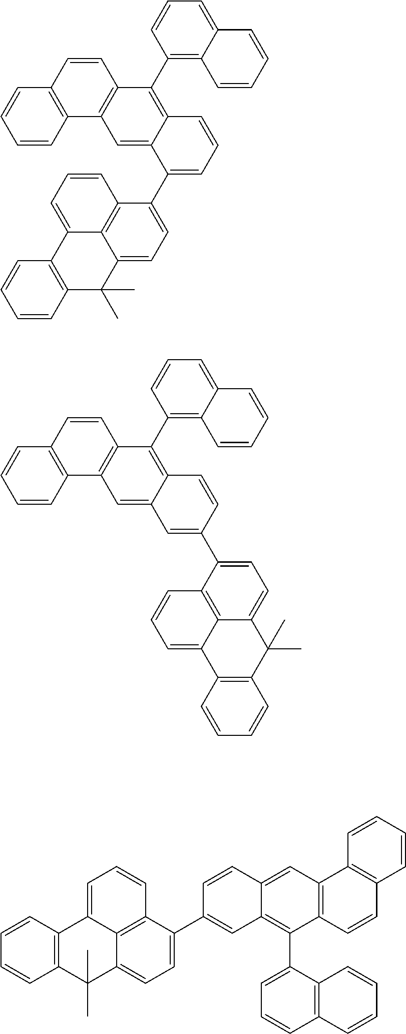

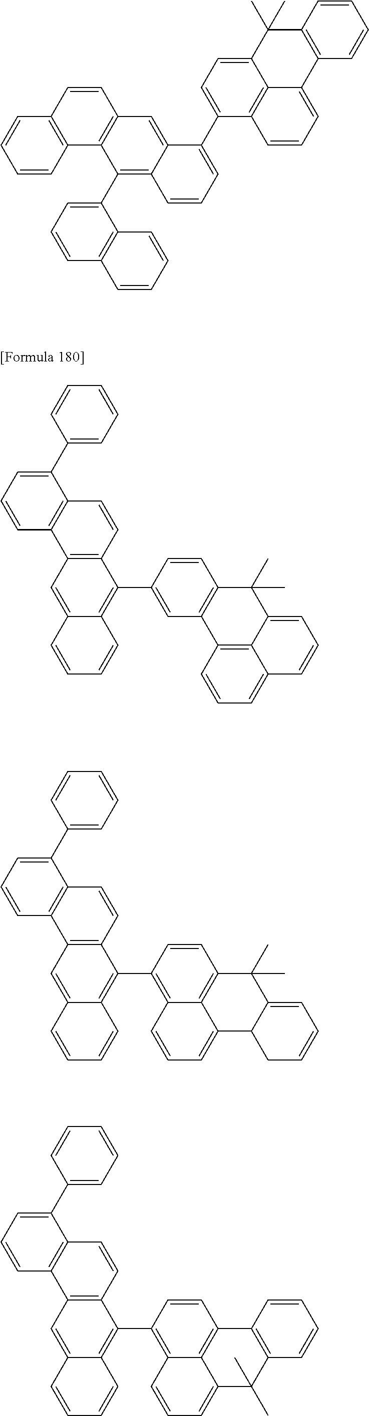

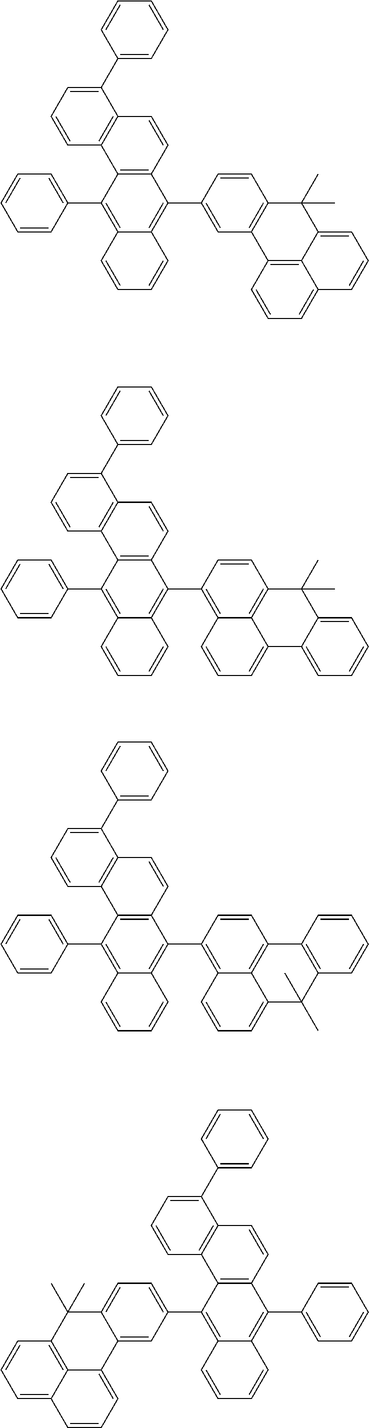

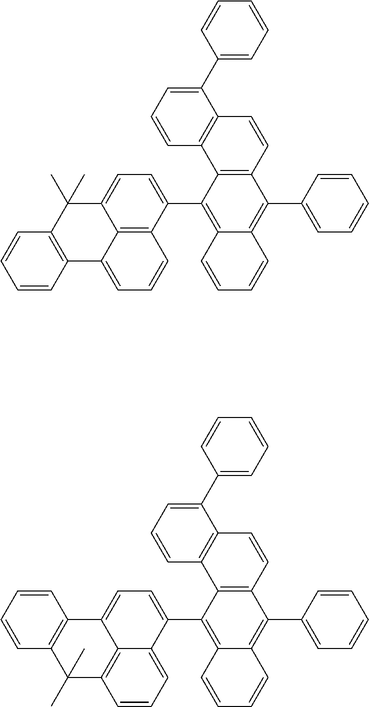

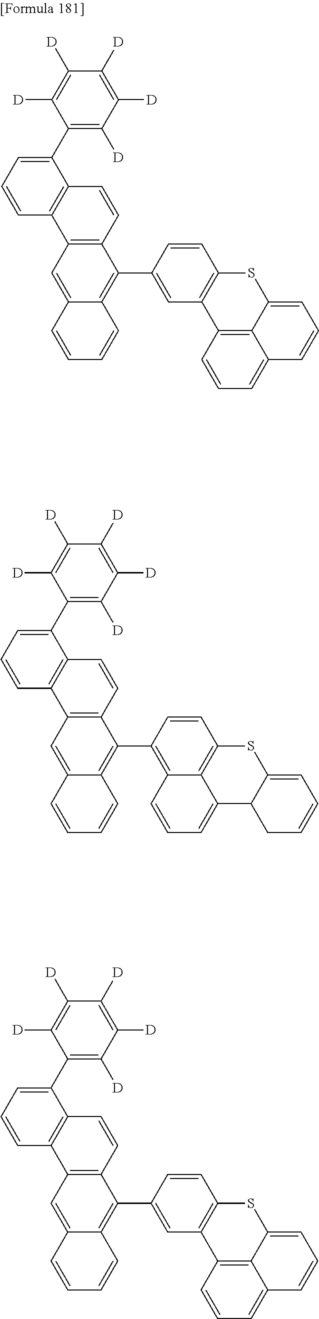 [SMILES: [2H]c1c([2H])c([2H])c(-c2cccc3c2ccc2c(-c4ccc5c(c4)-c4cccc6cccc(c46)S5)c4ccccc4cc23)c([2H])c1[2H] ; [2H]c1c([2H])c([2H])c(-c2cccc3c2ccc2c(-c4ccc5c6c(cccc46)C4CC=CC=C4S5)c4ccccc4cc23)c([2H])c1[2H] ; [2H]c1c([2H])c([2H])c(-c2cccc3c2ccc2c(-c4ccc5c(c4)-c4cccc6cccc(c46)S5)c4ccccc4cc23)c([2H])c1[2H]]

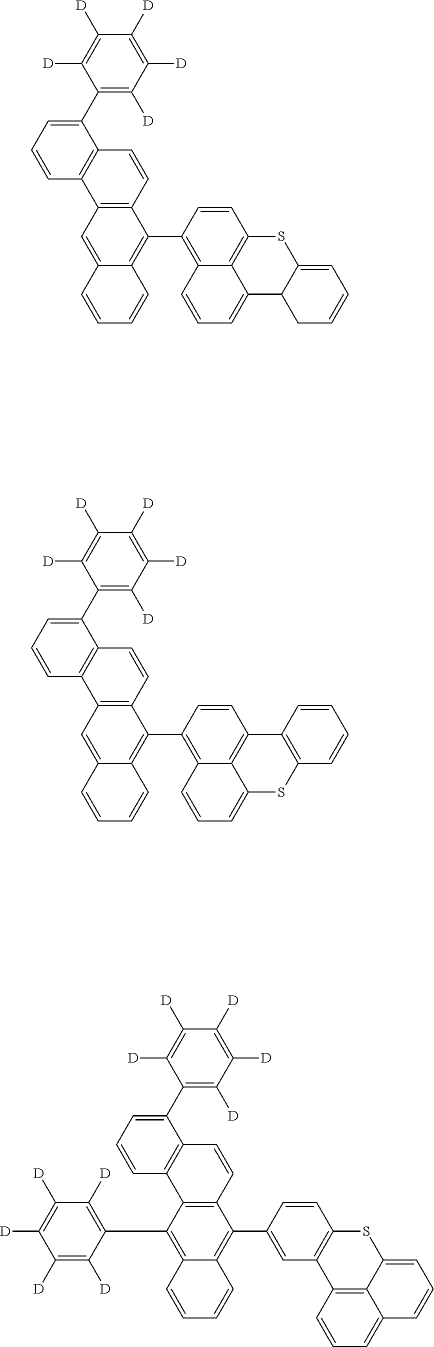 [SMILES: [2H]c1c([2H])c([2H])c(-c2cccc3c2ccc2c(-c4ccc5c6c(cccc46)C4CC=CC=C4S5)c4ccccc4cc23)c([2H])c1[2H] ; [2H]c1c([2H])c([2H])c(-c2cccc3c2ccc2c(-c4ccc5c6c(cccc46)Sc4ccccc4-5)c4ccccc4cc23)c([2H])c1[2H] ; [2H]c1c([2H])c([2H])c(-c2cccc3c2ccc2c(-c4ccc5c(c4)-c4cccc6cccc(c46)S5)c4ccccc4c(-c4c([2H])c([2H])c([2H])c([2H])c4[2H])c23)c([2H])c1[2H]]

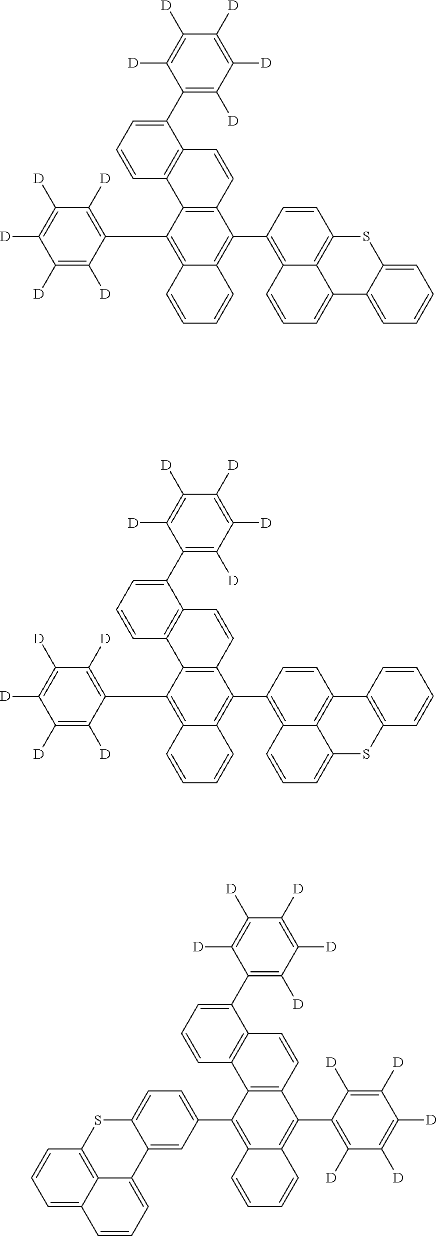 [SMILES: [2H]c1c([2H])c([2H])c(-c2cccc3c2ccc2c(-c4ccc5c6c(cccc46)-c4ccccc4S5)c4ccccc4c(-c4c([2H])c([2H])c([2H])c([2H])c4[2H])c23)c([2H])c1[2H] ; [2H]c1c([2H])c([2H])c(-c2cccc3c2ccc2c(-c4ccc5c6c(cccc46)Sc4ccccc4-5)c4ccccc4c(-c4c([2H])c([2H])c([2H])c([2H])c4[2H])c23)c([2H])c1[2H] ; [2H]c1c([2H])c([2H])c(-c2cccc3c2ccc2c(-c4c([2H])c([2H])c([2H])c([2H])c4[2H])c4ccccc4c(-c4ccc5c(c4)-c4cccc6cccc(c46)S5)c23)c([2H])c1[2H]]

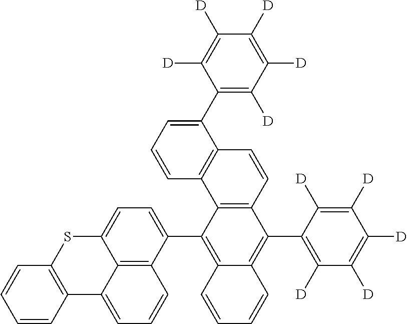 [SMILES: [2H]c1c([2H])c([2H])c(-c2cccc3c2ccc2c(-c4c([2H])c([2H])c([2H])c([2H])c4[2H])c4ccccc4c(-c4ccc5c6c(cccc46)-c4ccccc4S5)c23)c([2H])c1[2H]]

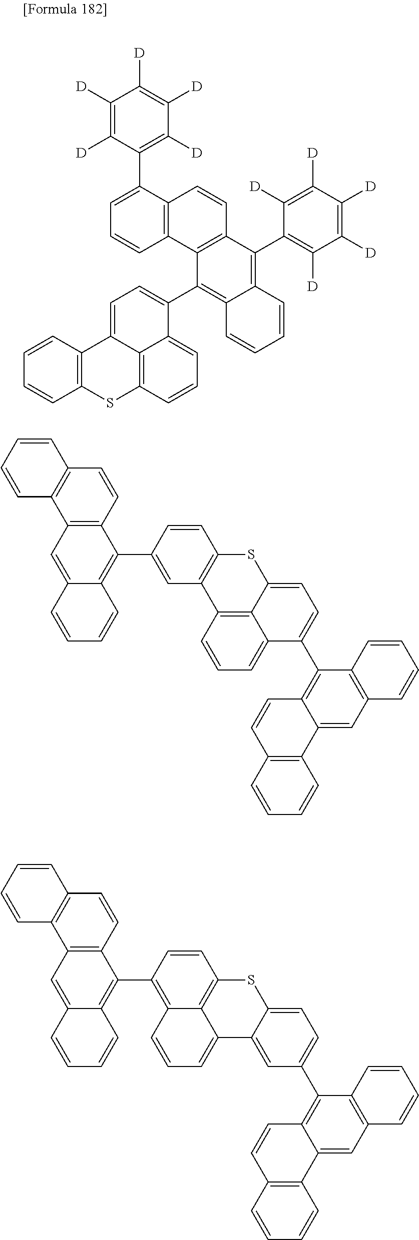

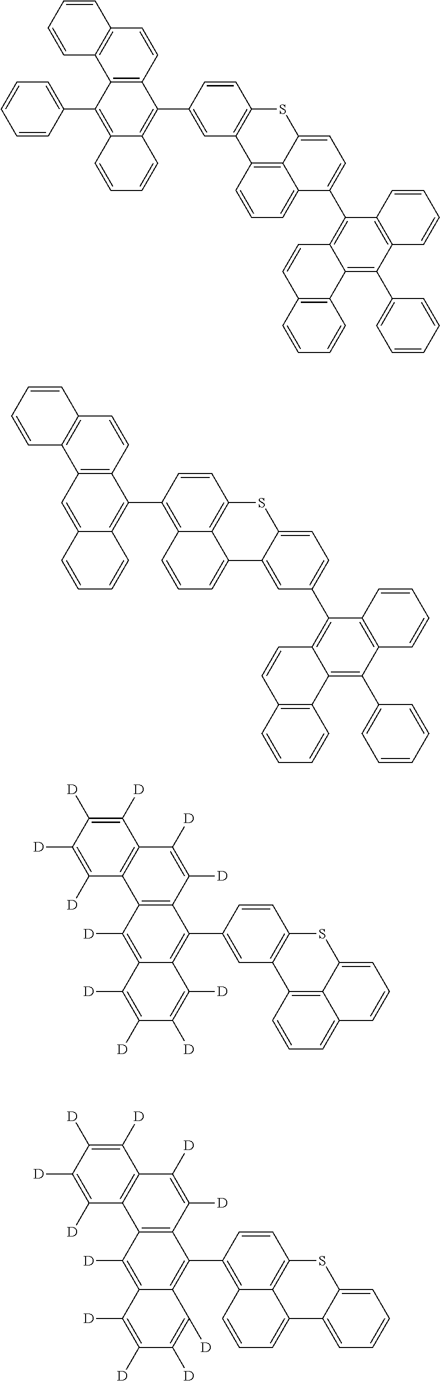 [SMILES: c1ccc(-c2c3ccccc3c(-c3ccc4c(c3)-c3cccc5c(-c6c7ccccc7c(-c7ccccc7)c7c6ccc6ccccc67)ccc(c35)S4)c3ccc4ccccc4c23)cc1 ; c1ccc(-c2c3ccccc3c(-c3ccc4c(c3)-c3cccc5c(-c6c7ccccc7cc7c6ccc6ccccc67)ccc(c35)S4)c3ccc4ccccc4c23)cc1 ; [2H]c1c([2H])c([2H])c2c([2H])c3c(c([2H])c([2H])c4c([2H])c([2H])c([2H])c([2H])c43)c(-c3ccc4c(c3)-c3cccc5cccc(c35)S4)c2c1[2H] ; [2H]c1c([2H])c([2H])c2c([2H])c3c(c([2H])c([2H])c4c([2H])c([2H])c([2H])c([2H])c43)c(-c3ccc4c5c(cccc35)-c3ccccc3S4)c2c1[2H]]

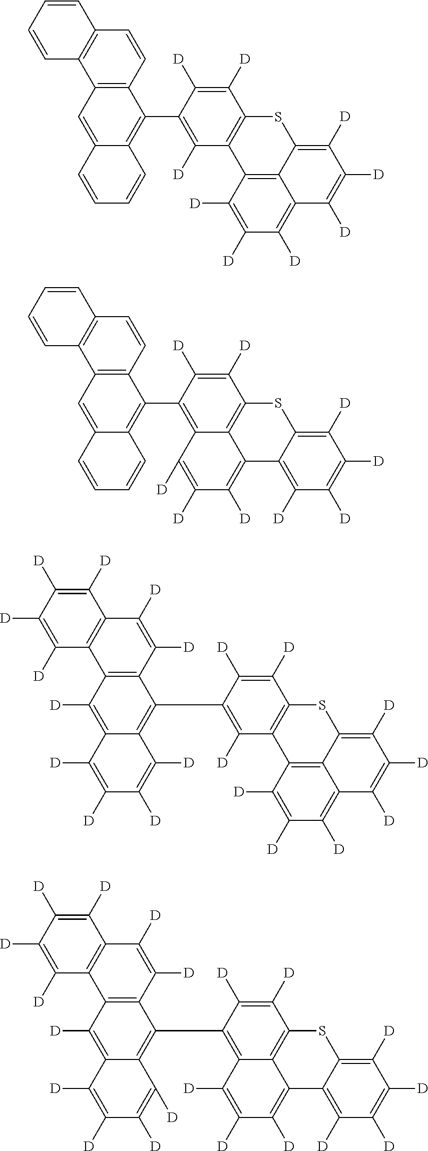

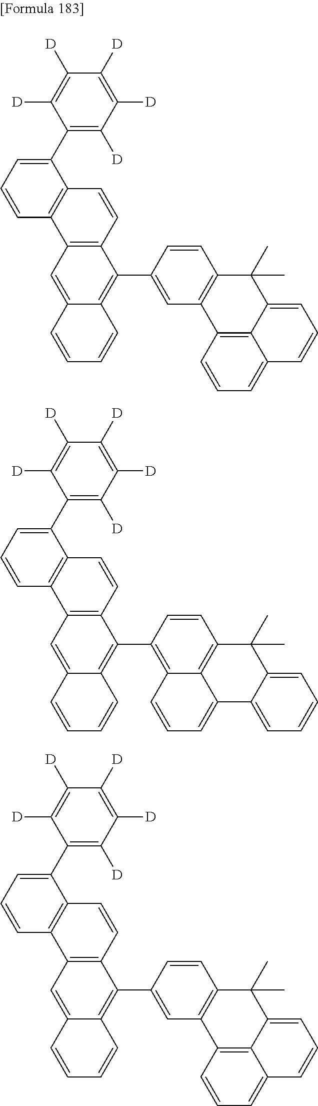

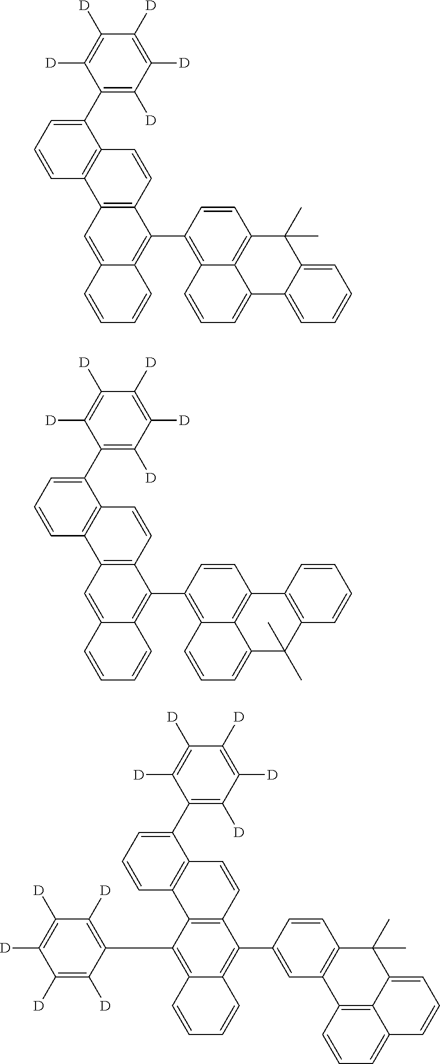

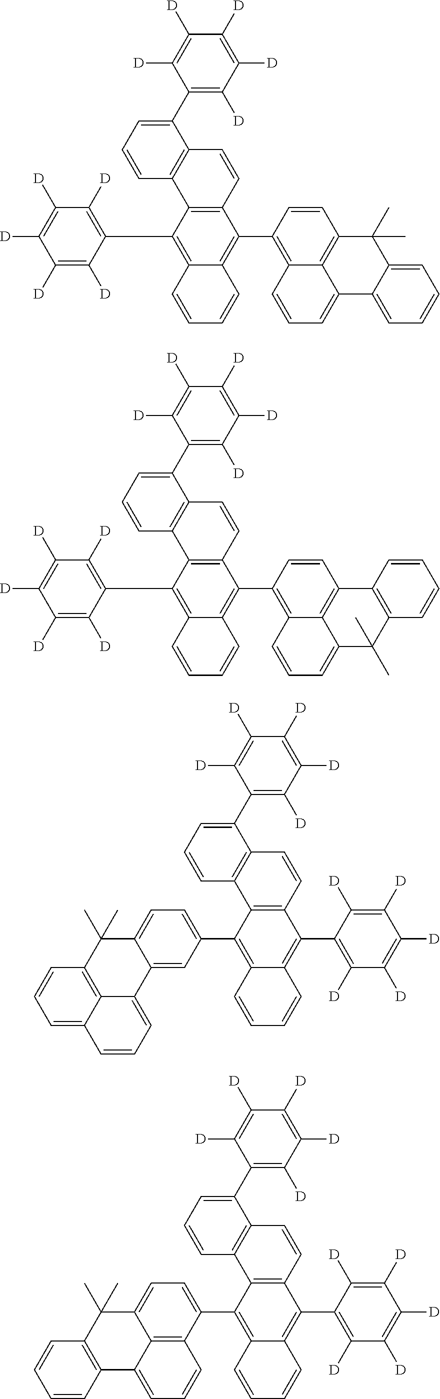

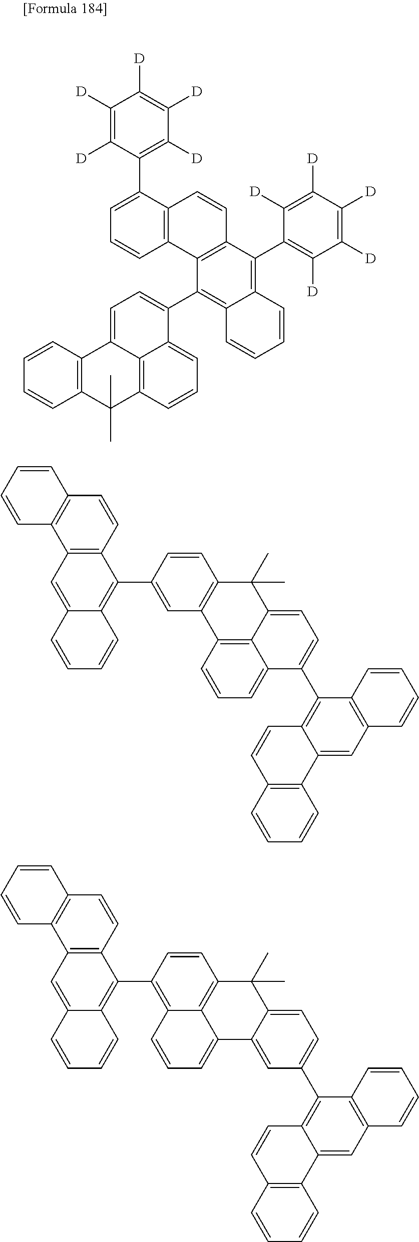

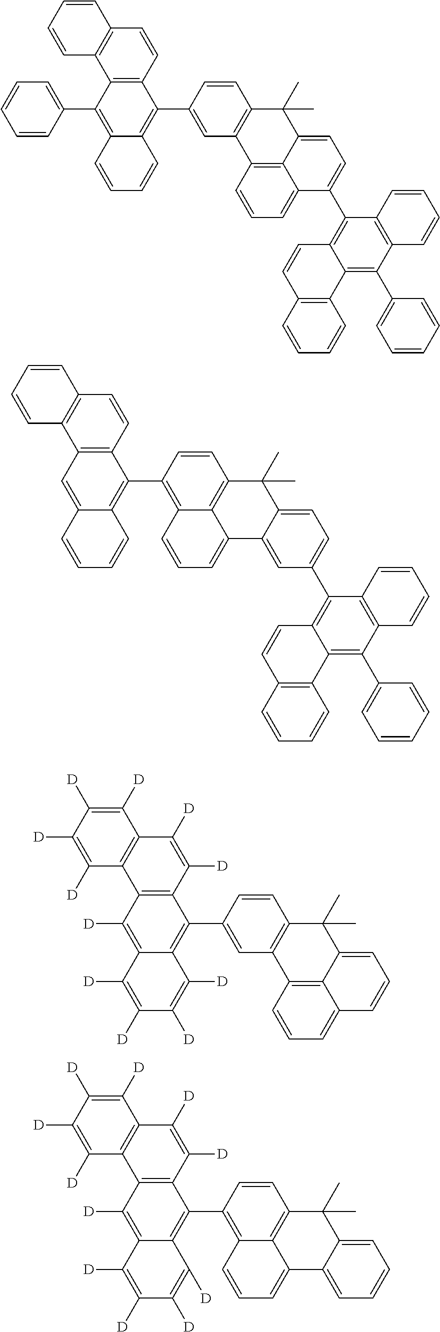

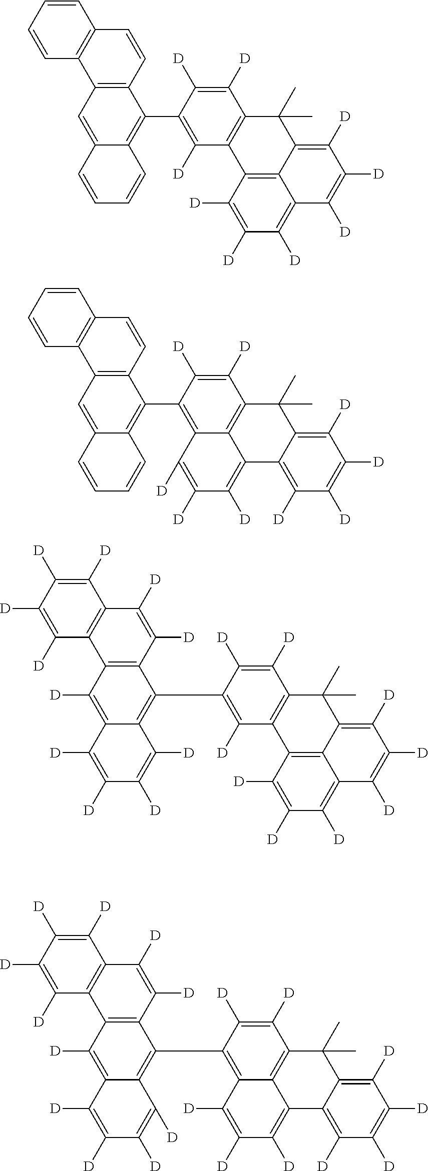

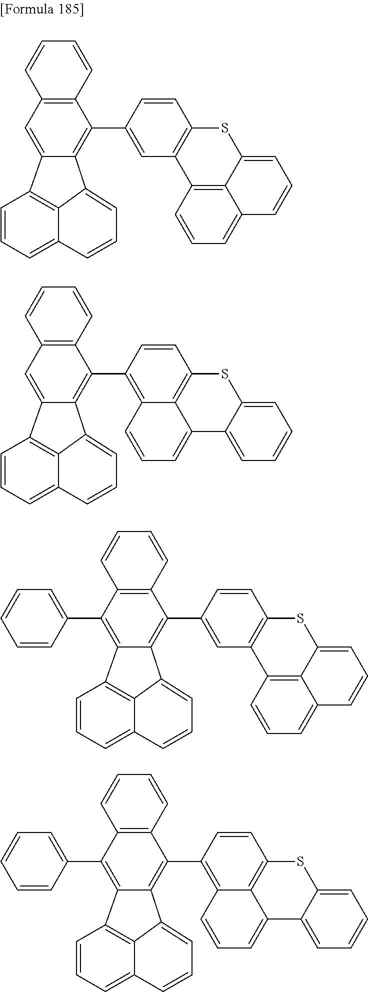

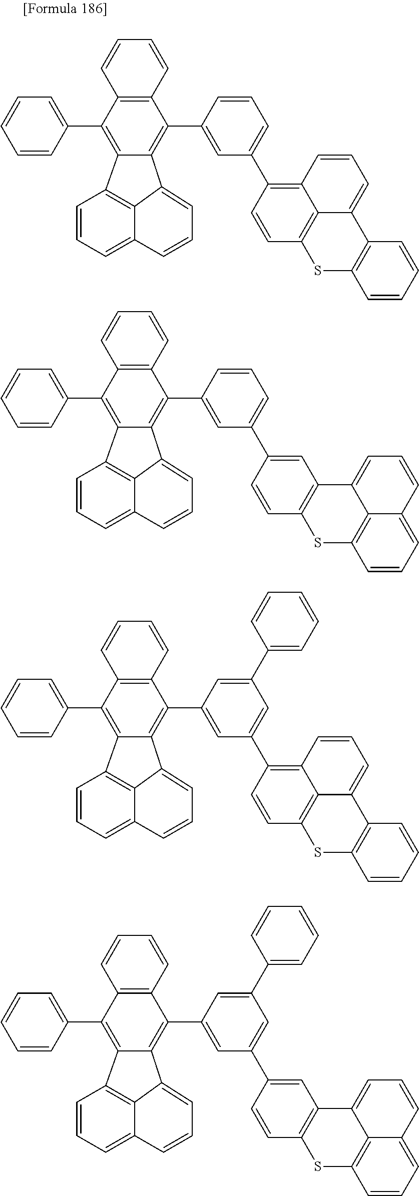 [SMILES: c1ccc(-c2c3c(c(-c4cccc(-c5ccc6c7c(cccc57)-c5ccccc5S6)c4)c4ccccc24)-c2cccc4cccc-3c24)cc1 ; c1ccc(-c2c3c(c(-c4cccc(-c5ccc6c(c5)-c5cccc7cccc(c57)S6)c4)c4ccccc24)-c2cccc4cccc-3c24)cc1 ; c1ccc(-c2cc(-c3c4c(c(-c5ccccc5)c5ccccc35)-c3cccc5cccc-4c35)cc(-c3ccc4c5c(cccc35)-c3ccccc3S4)c2)cc1 ; c1ccc(-c2cc(-c3ccc4c(c3)-c3cccc5cccc(c35)S4)cc(-c3c4c(c(-c5ccccc5)c5ccccc35)-c3cccc5cccc-4c35)c2)cc1]

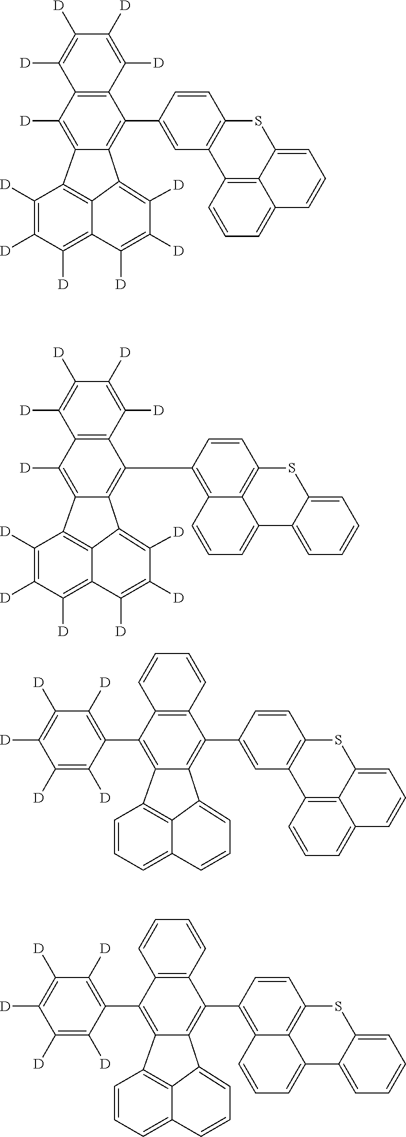

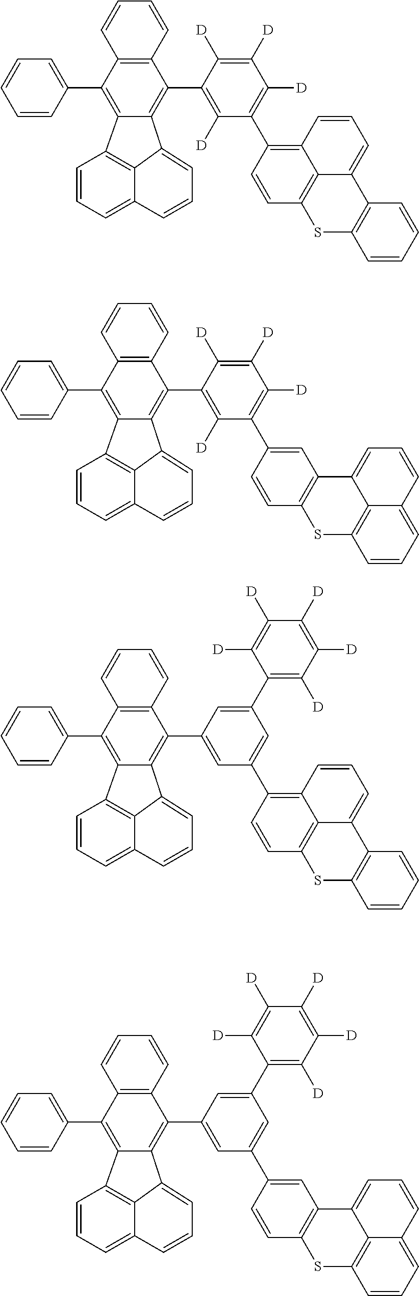 [SMILES: [2H]c1c([2H])c(-c2c3c(c(-c4ccccc4)c4ccccc24)-c2cccc4cccc-3c24)c([2H])c(-c2ccc3c4c(cccc24)-c2ccccc2S3)c1[2H] ; [2H]c1c([2H])c(-c2ccc3c(c2)-c2cccc4cccc(c24)S3)c([2H])c(-c2c3c(c(-c4ccccc4)c4ccccc24)-c2cccc4cccc-3c24)c1[2H] ; [2H]c1c([2H])c([2H])c(-c2cc(-c3c4c(c(-c5ccccc5)c5ccccc35)-c3cccc5cccc-4c35)cc(-c3ccc4c5c(cccc35)-c3ccccc3S4)c2)c([2H])c1[2H] ; [2H]c1c([2H])c([2H])c(-c2cc(-c3ccc4c(c3)-c3cccc5cccc(c35)S4)cc(-c3c4c(c(-c5ccccc5)c5ccccc35)-c3cccc5cccc-4c35)c2)c([2H])c1[2H]]

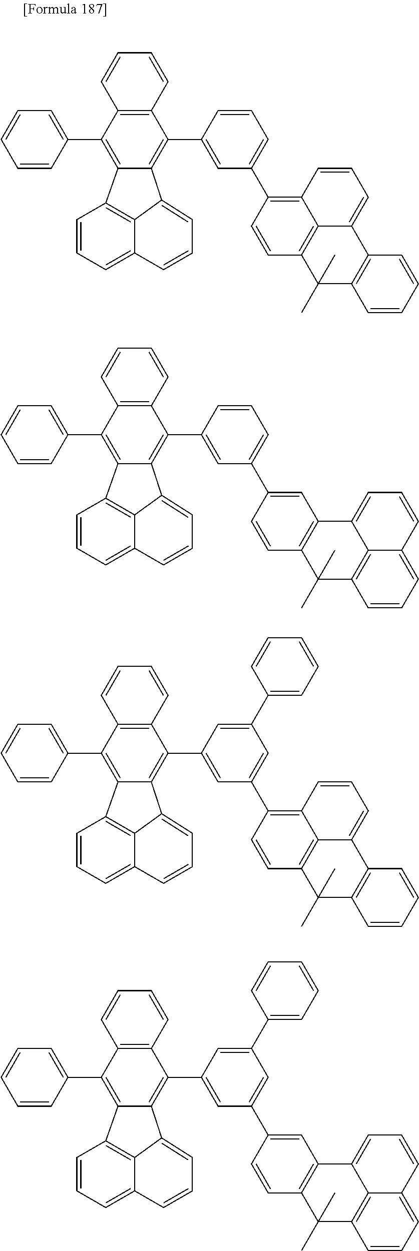

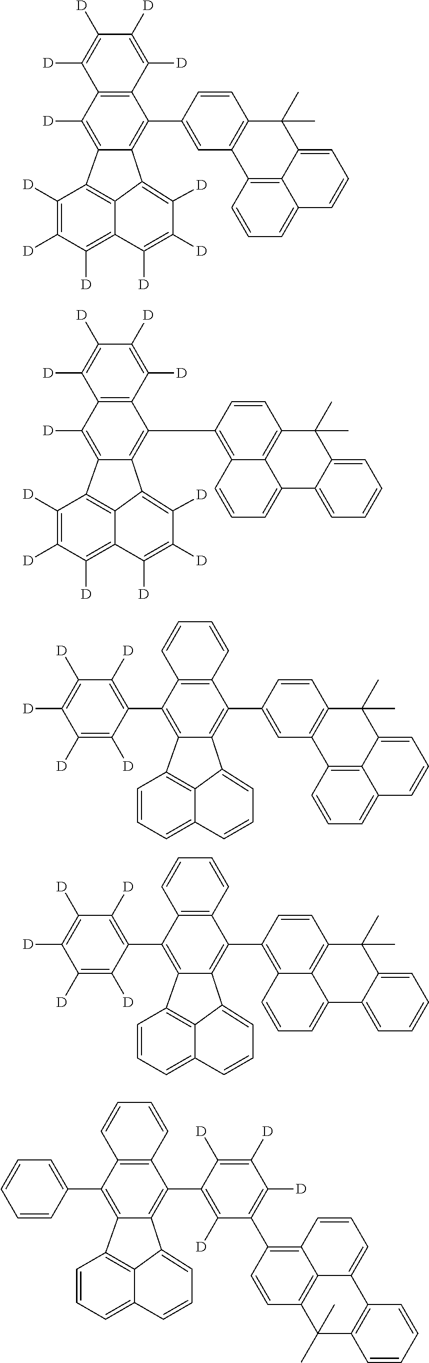

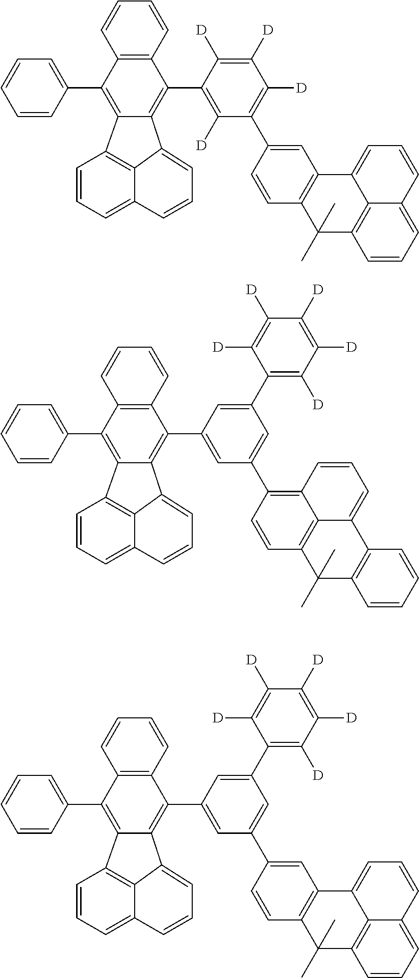

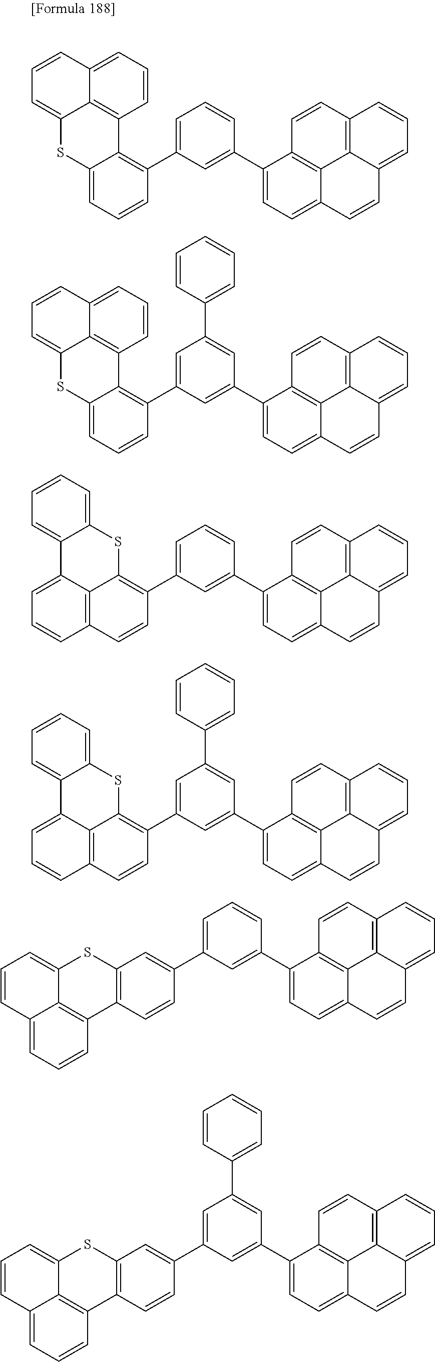

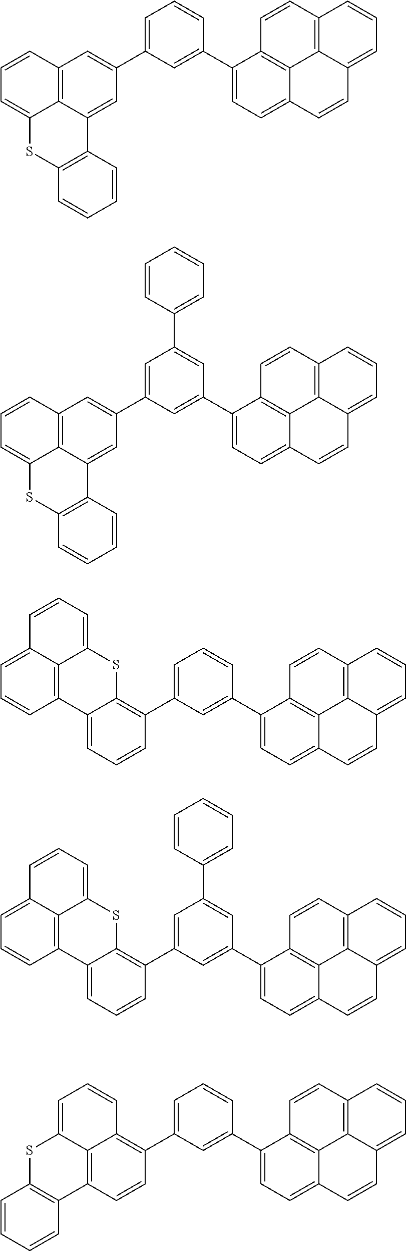

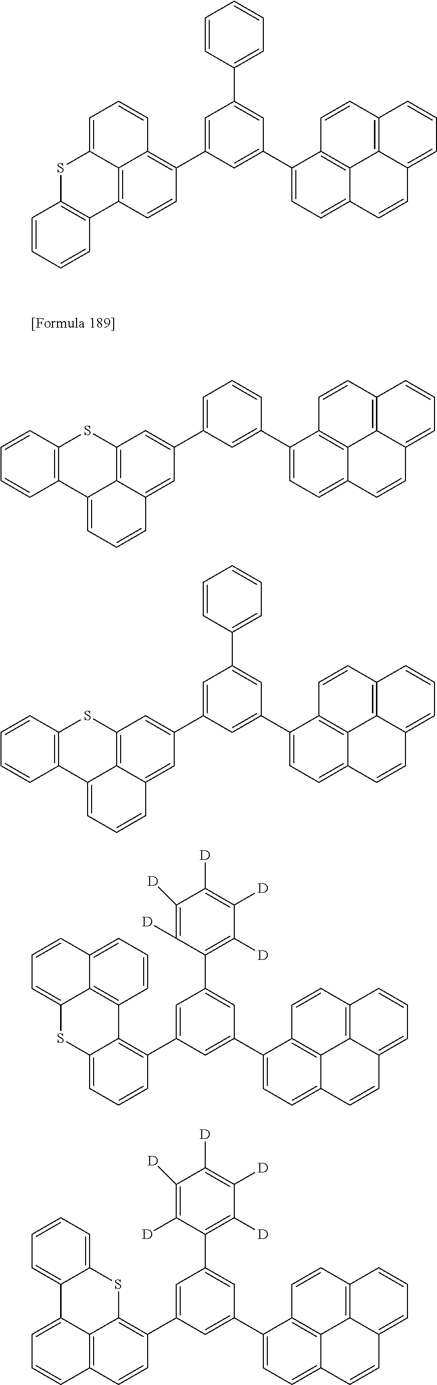 [SMILES: c1ccc(-c2cc(-c3ccc4c5c(cccc35)Sc3ccccc3-4)cc(-c3ccc4ccc5cccc6ccc3c4c56)c2)cc1 ; c1cc(-c2cc3c4c(cccc4c2)-c2ccccc2S3)cc(-c2ccc3ccc4cccc5ccc2c3c45)c1 ; c1ccc(-c2cc(-c3cc4c5c(cccc5c3)-c3ccccc3S4)cc(-c3ccc4ccc5cccc6ccc3c4c56)c2)cc1 ; [2H]c1c([2H])c([2H])c(-c2cc(-c3cccc4c3-c3cccc5cccc(c35)S4)cc(-c3ccc4ccc5cccc6ccc3c4c56)c2)c([2H])c1[2H] ; [2H]c1c([2H])c([2H])c(-c2cc(-c3ccc4cccc5c4c3Sc3ccccc3-5)cc(-c3ccc4ccc5cccc6ccc3c4c56)c2)c([2H])c1[2H]]

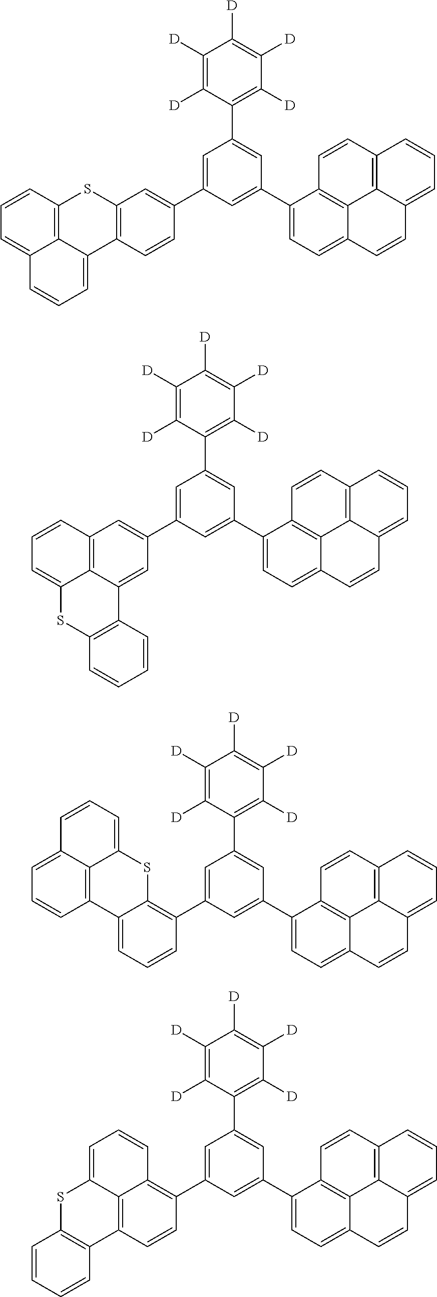 [SMILES: [2H]c1c([2H])c([2H])c(-c2cc(-c3ccc4c(c3)Sc3cccc5cccc-4c35)cc(-c3ccc4ccc5cccc6ccc3c4c56)c2)c([2H])c1[2H] ; [2H]c1c([2H])c([2H])c(-c2cc(-c3cc4c5c(cccc5c3)Sc3ccccc3-4)cc(-c3ccc4ccc5cccc6ccc3c4c56)c2)c([2H])c1[2H] ; [2H]c1c([2H])c([2H])c(-c2cc(-c3cccc4c3Sc3cccc5cccc-4c35)cc(-c3ccc4ccc5cccc6ccc3c4c56)c2)c([2H])c1[2H] ; [2H]c1c([2H])c([2H])c(-c2cc(-c3ccc4c5c(cccc35)Sc3ccccc3-4)cc(-c3ccc4ccc5cccc6ccc3c4c56)c2)c([2H])c1[2H]]

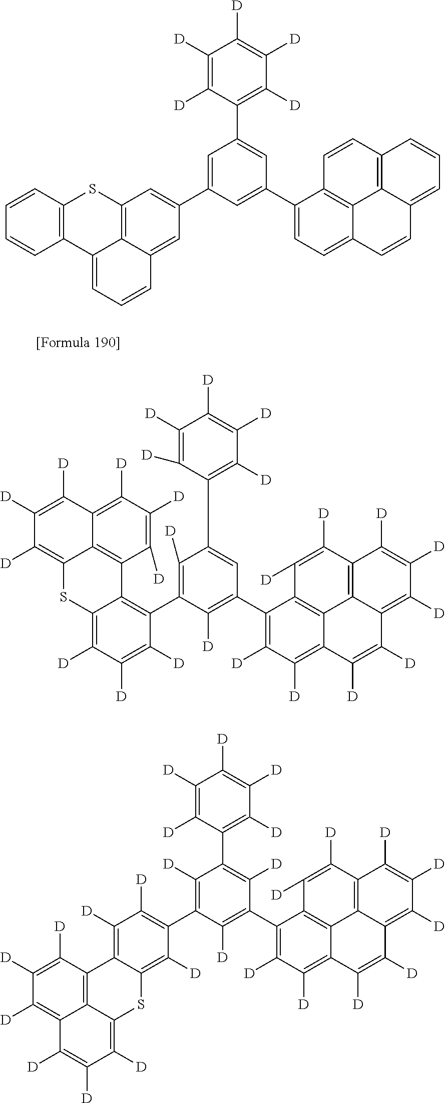

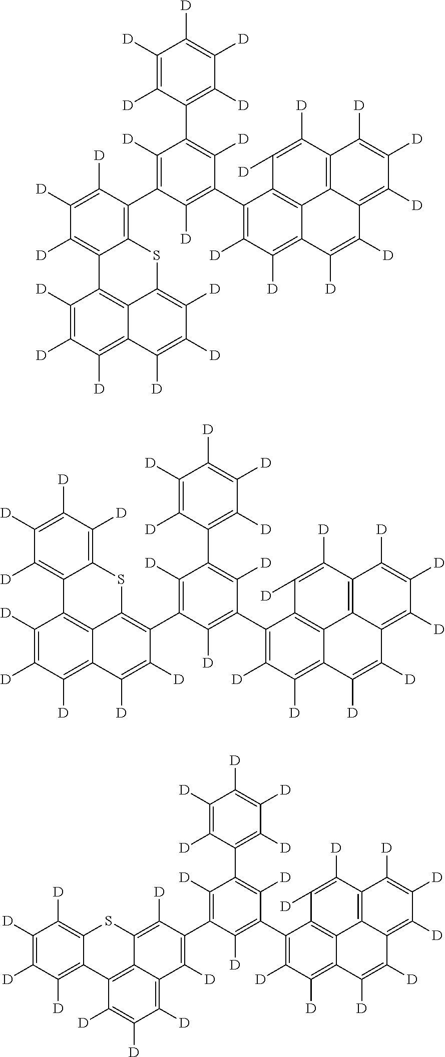

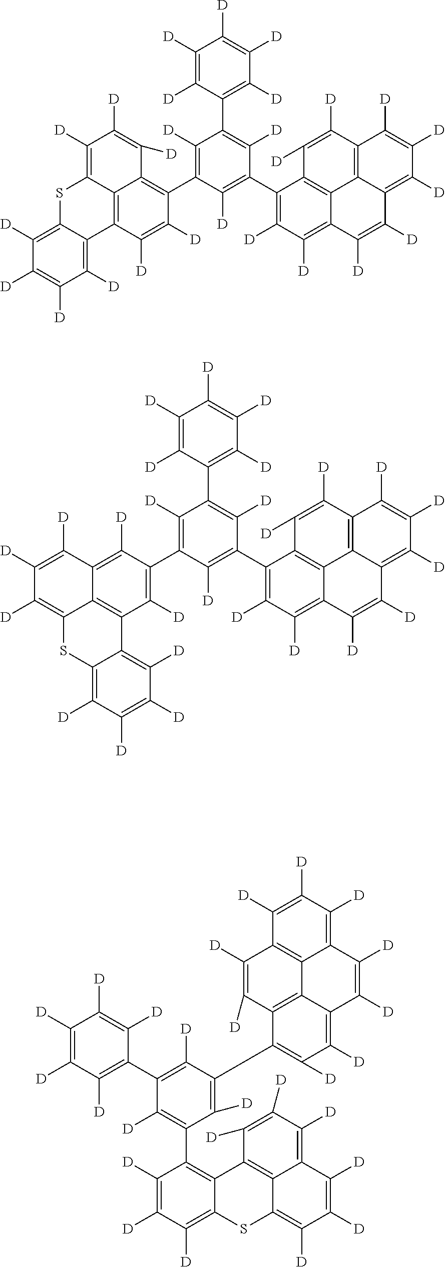

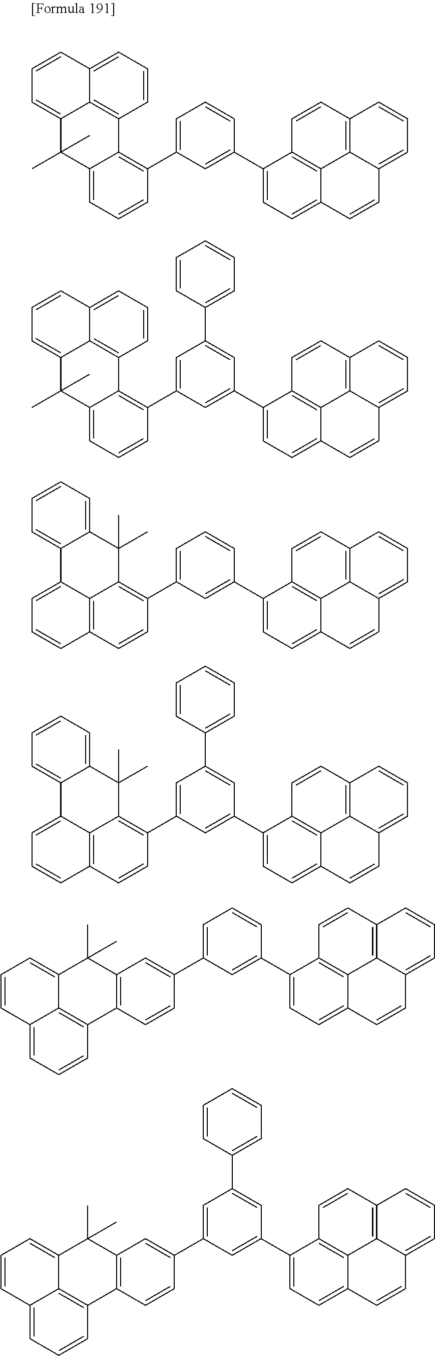

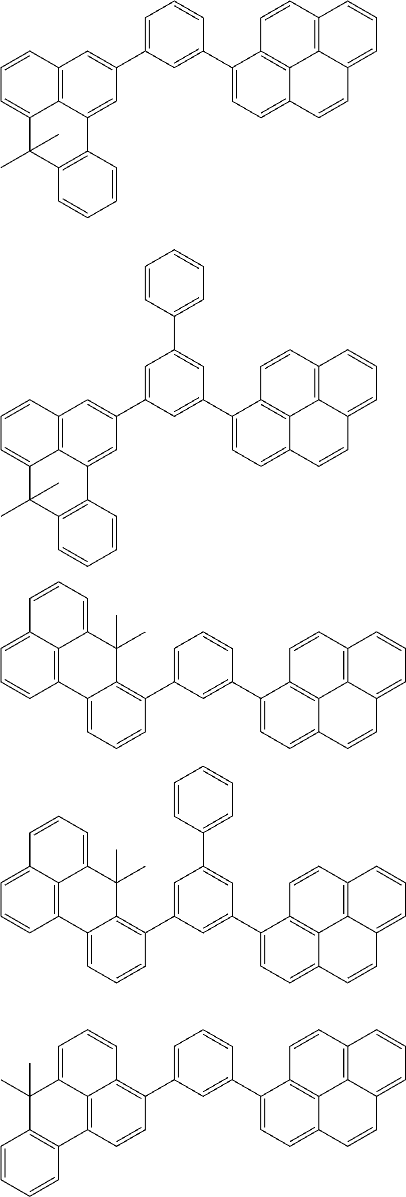

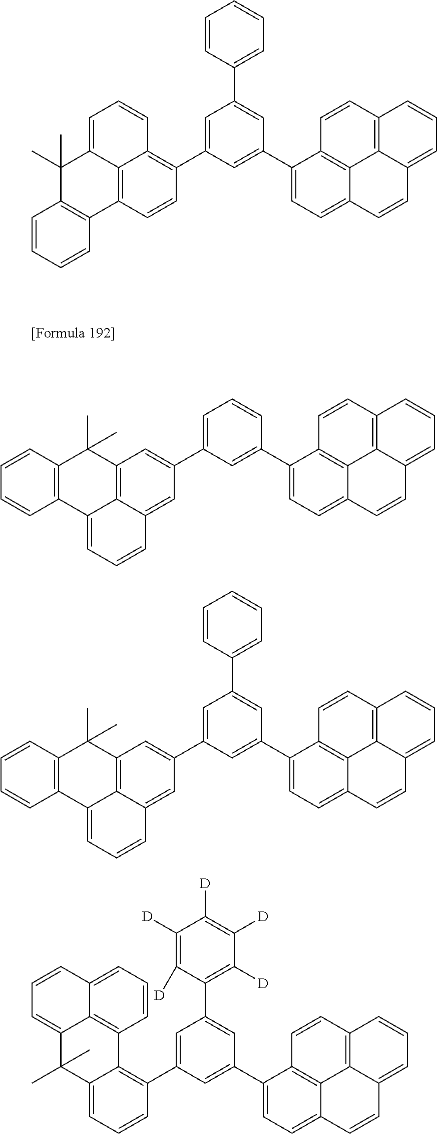

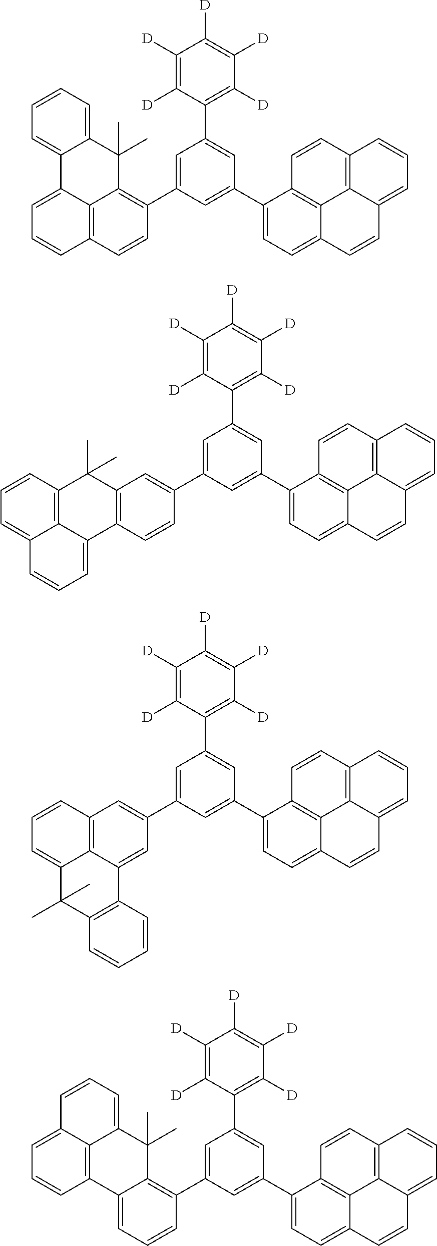

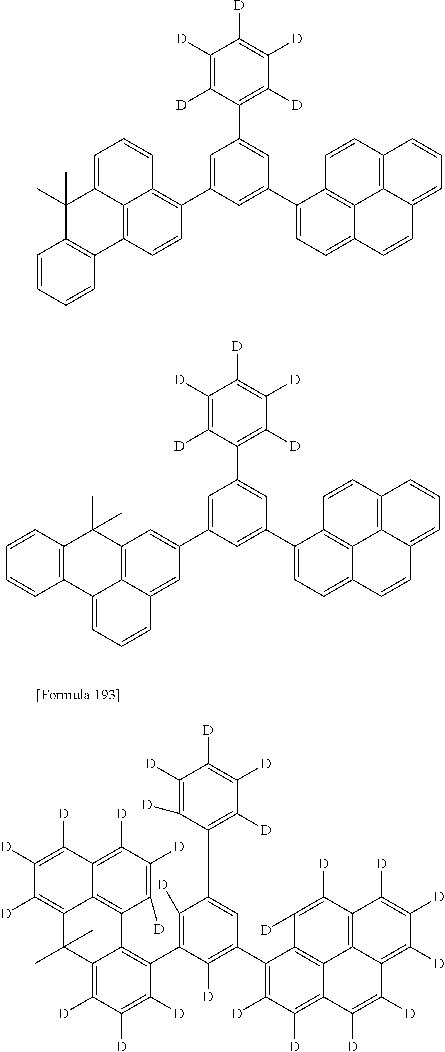

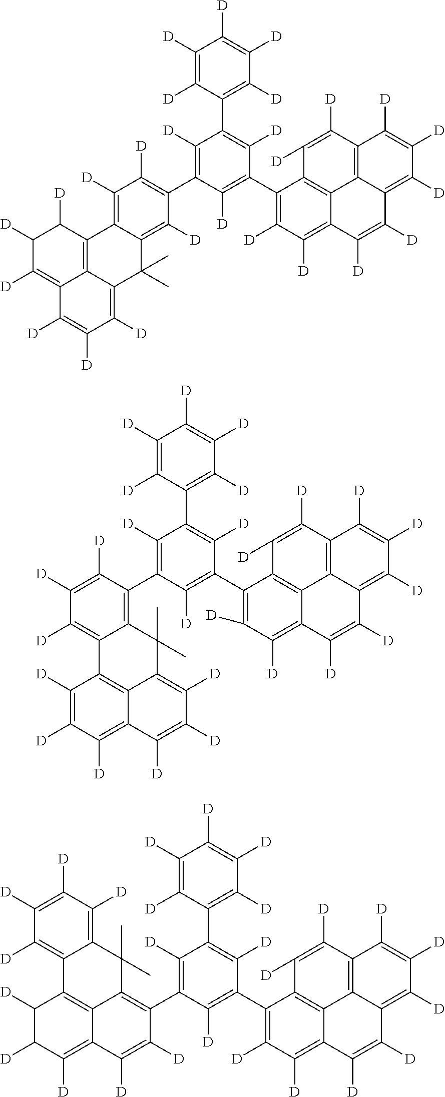

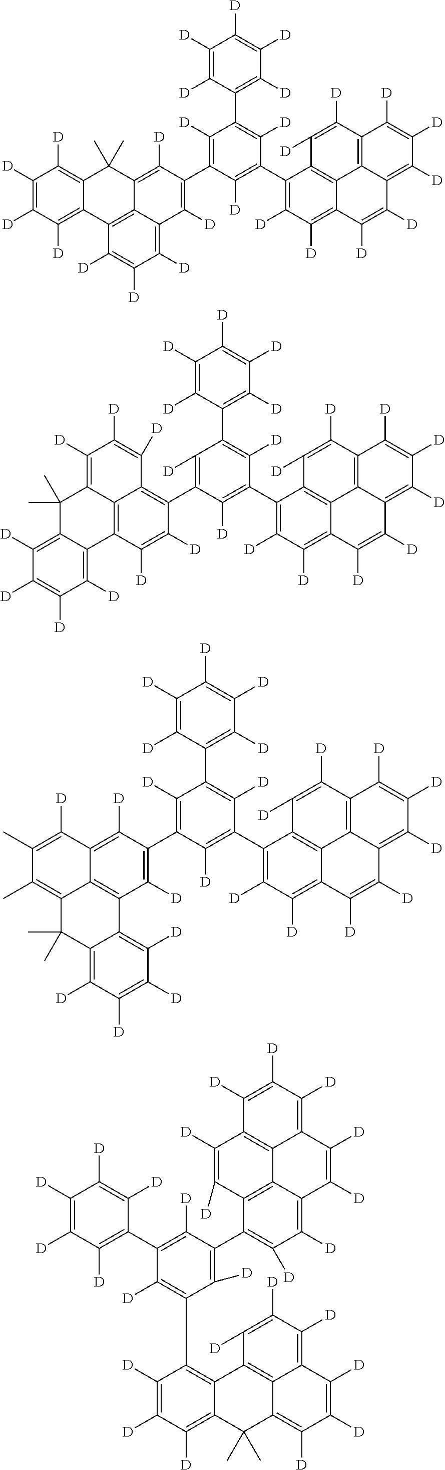

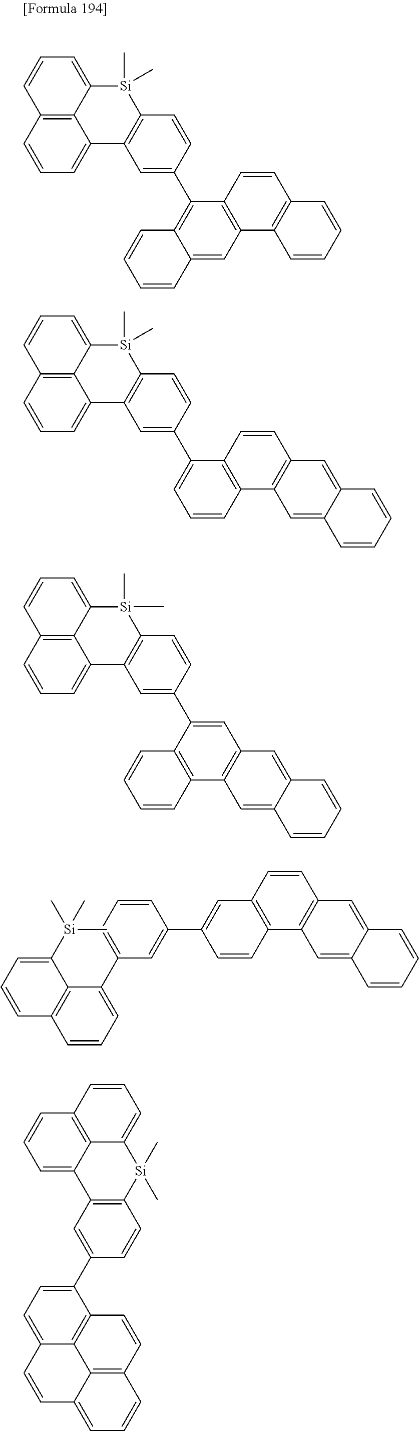

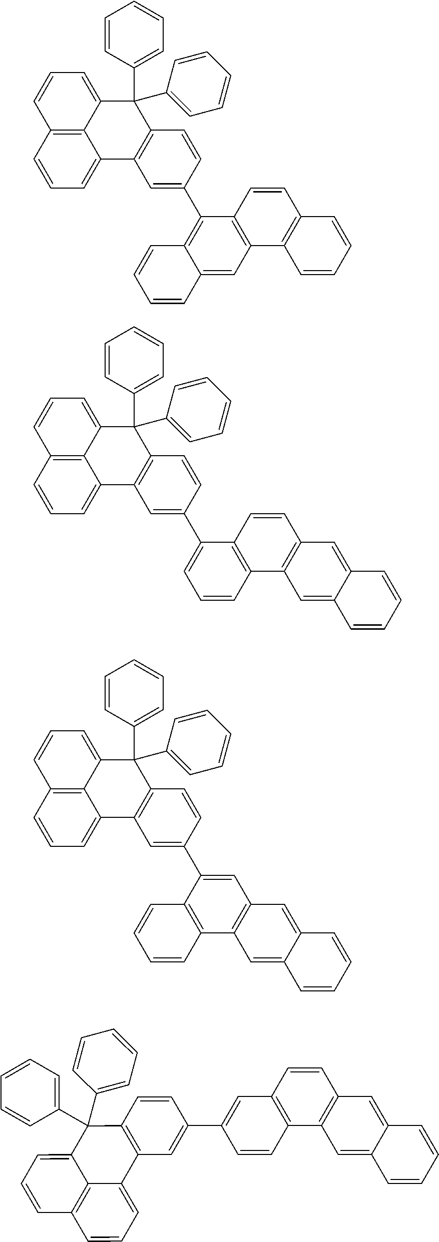

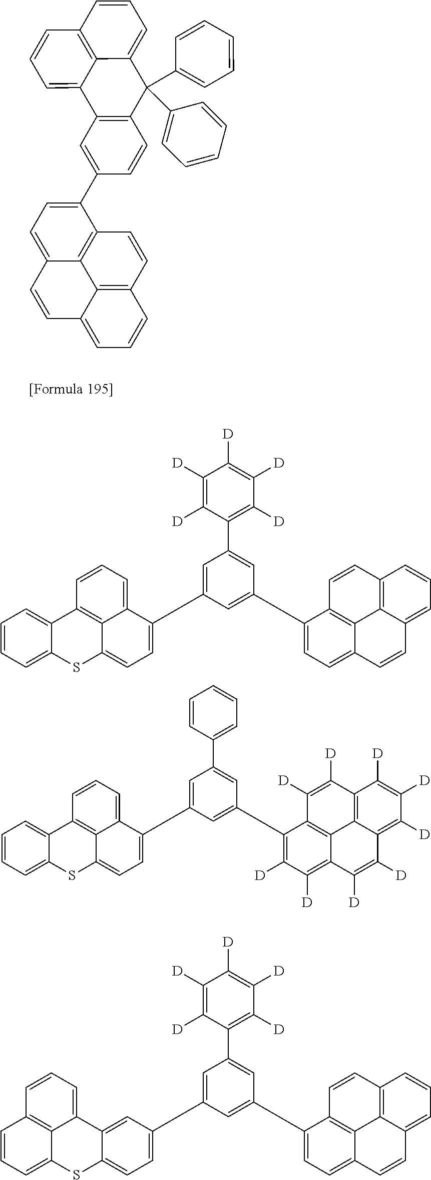

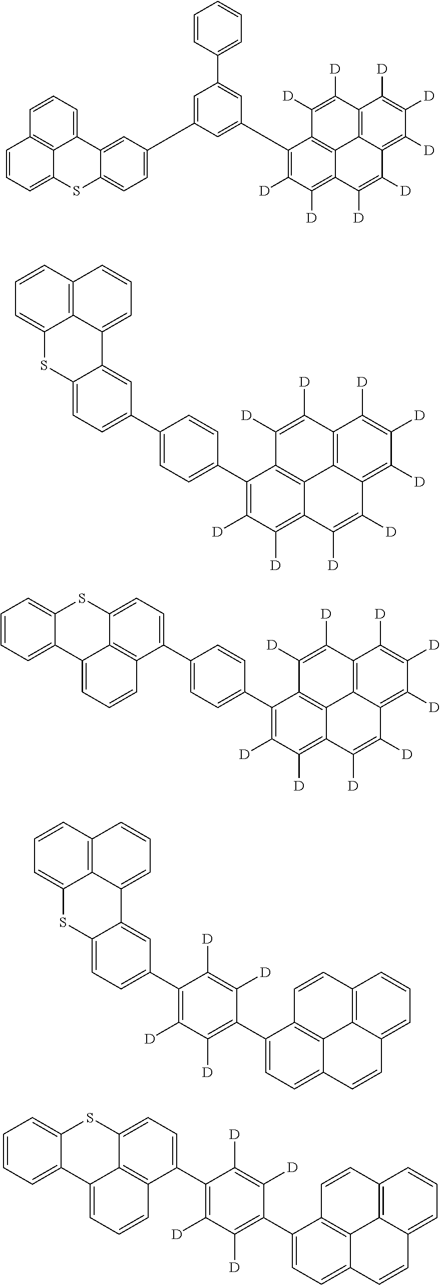

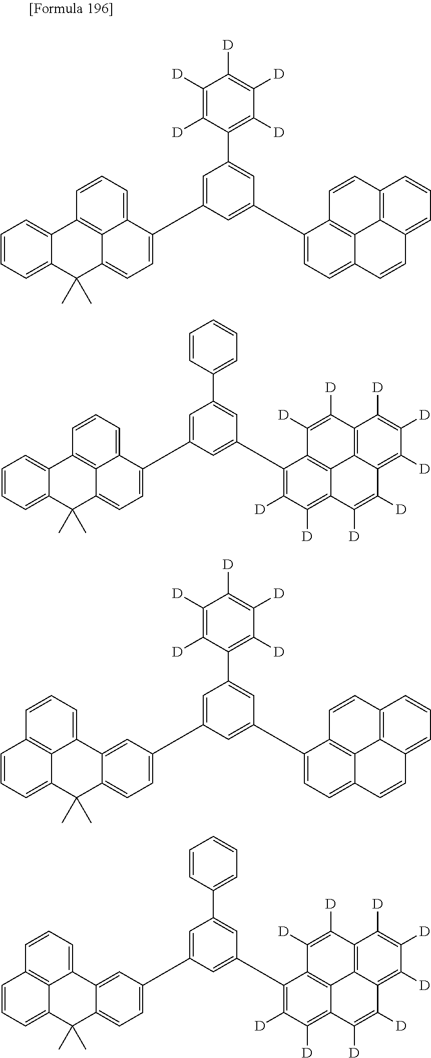 [SMILES: [2H]c1c([2H])c([2H])c(-c2cc(Cc3ccc4c5c(cccc35)-c3ccccc3C4(C)C)cc(Cc3ccc4ccc5cccc6ccc3c4c56)c2)c([2H])c1[2H] ; [2H]c1c([2H])c2c([2H])c([2H])c3c([2H])c([2H])c(Cc4cc(Cc5ccc6c7c(cccc57)-c5ccccc5C6(C)C)cc(-c5ccccc5)c4)c4c([2H])c([2H])c(c1[2H])c2c34 ; [2H]c1c([2H])c([2H])c(-c2cc(Cc3ccc4c(c3)-c3cccc5cccc(c35)C4(C)C)cc(Cc3ccc4ccc5cccc6ccc3c4c56)c2)c([2H])c1[2H] ; [2H]c1c([2H])c2c([2H])c([2H])c3c([2H])c([2H])c(Cc4cc(Cc5ccc6c(c5)-c5cccc7cccc(c57)C6(C)C)cc(-c5ccccc5)c4)c4c([2H])c([2H])c(c1[2H])c2c34]

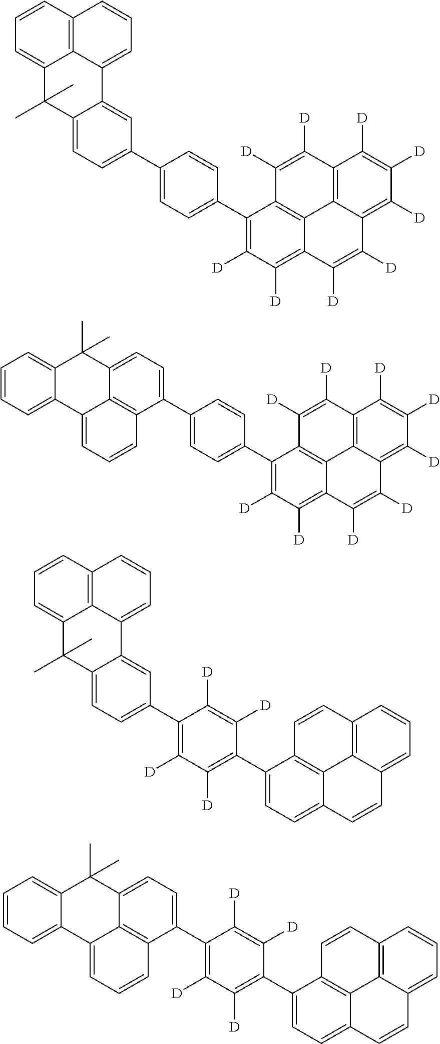

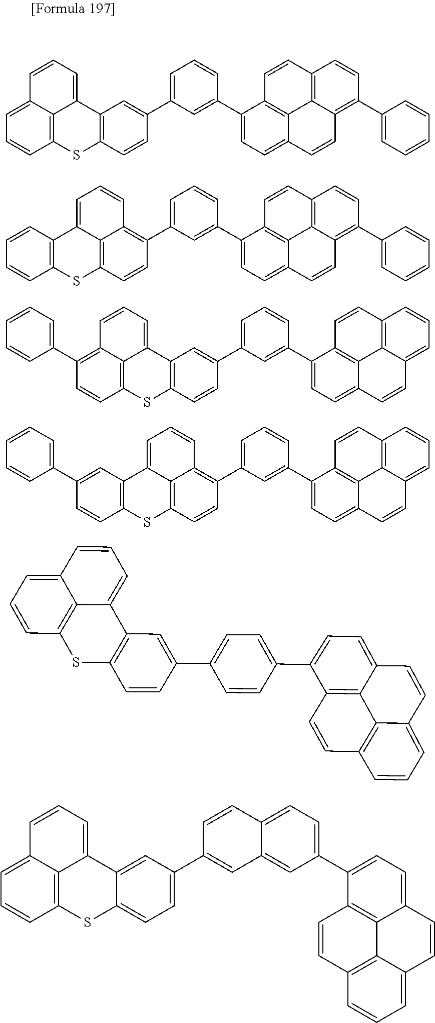

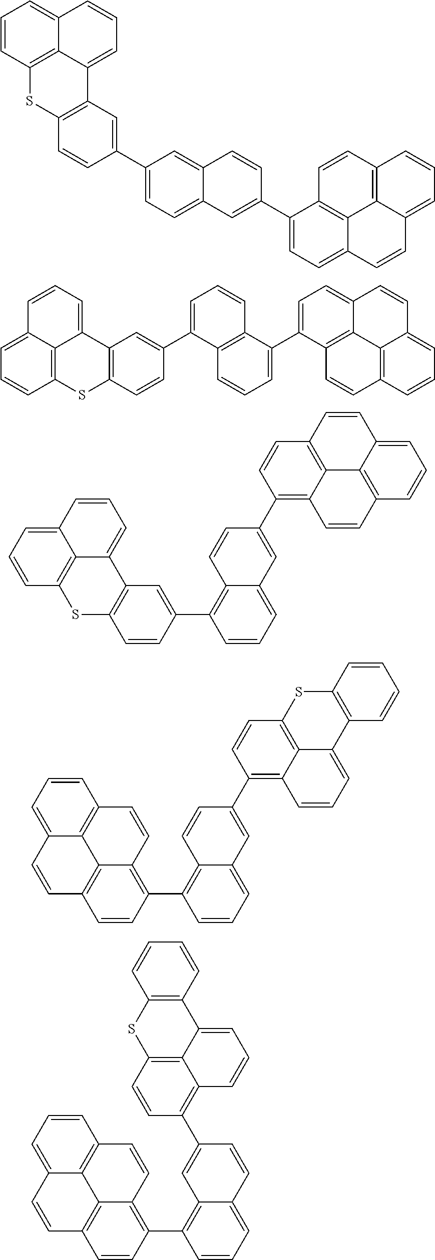

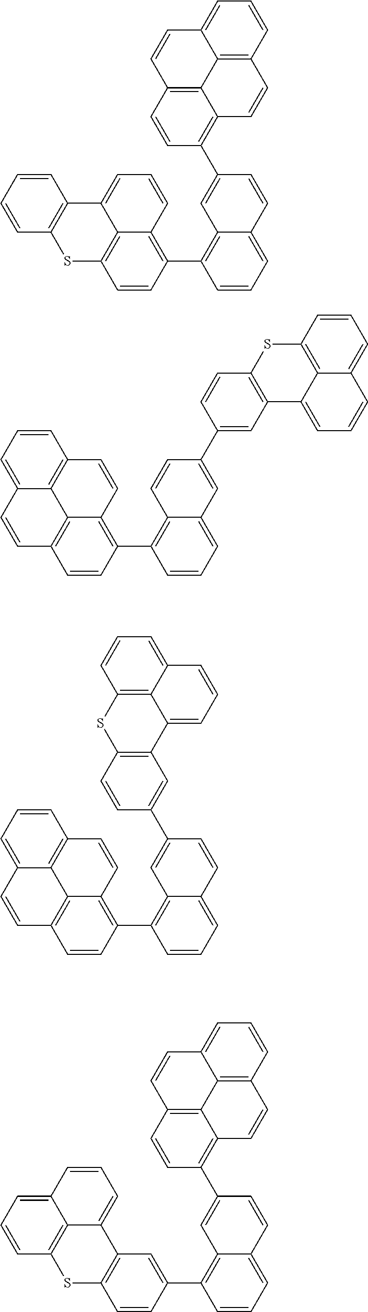 [SMILES: c1ccc2c(c1)Sc1ccc(-c3cccc4ccc(-c5ccc6ccc7cccc8ccc5c6c78)cc34)c3cccc-2c13 ; c1cc(-c2ccc3ccc4cccc5ccc2c3c45)c2ccc(-c3ccc4c(c3)-c3cccc5cccc(c35)S4)cc2c1 ; c1cc(-c2ccc3ccc4cccc5ccc2c3c45)c2cc(-c3ccc4c(c3)-c3cccc5cccc(c35)S4)ccc2c1 ; c1cc(-c2ccc3c(c2)-c2cccc4cccc(c24)S3)c2cc(-c3ccc4ccc5cccc6ccc3c4c56)ccc2c1]

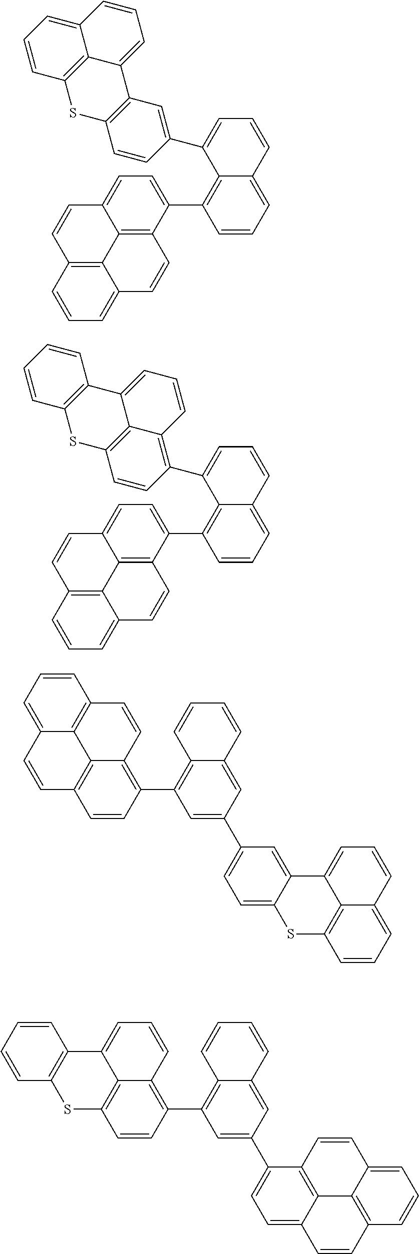

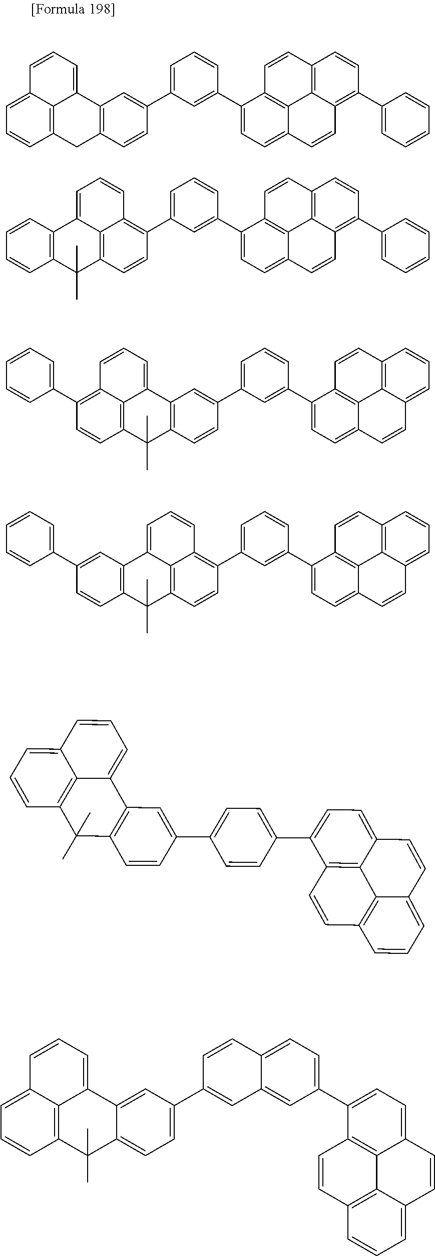

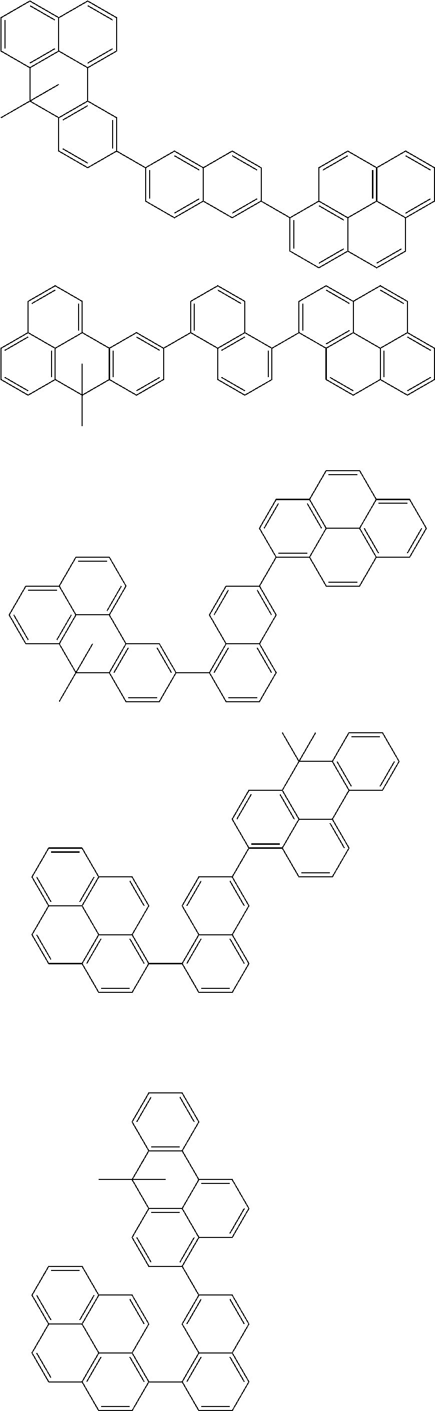

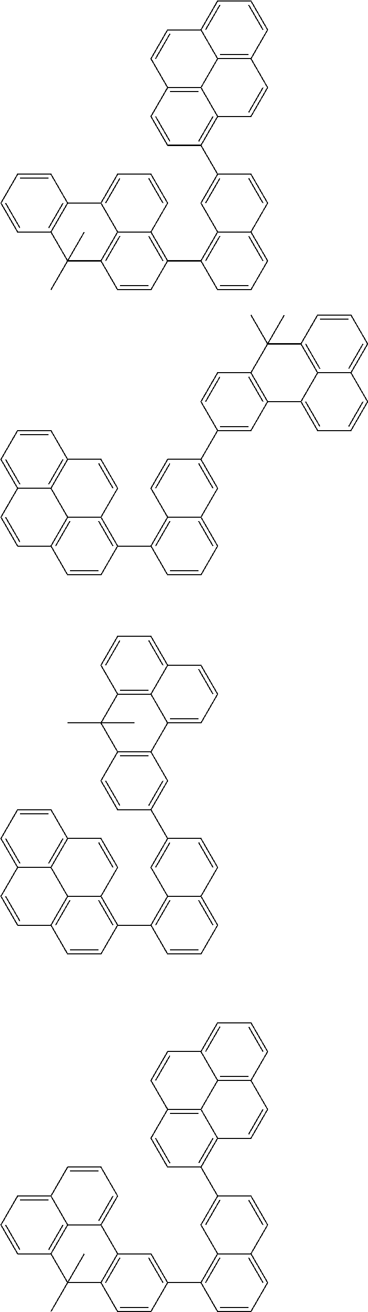

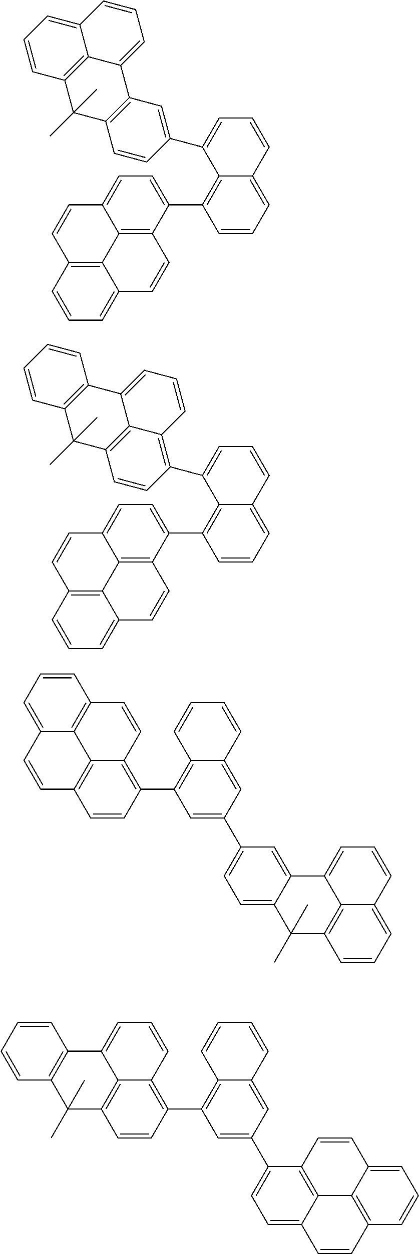

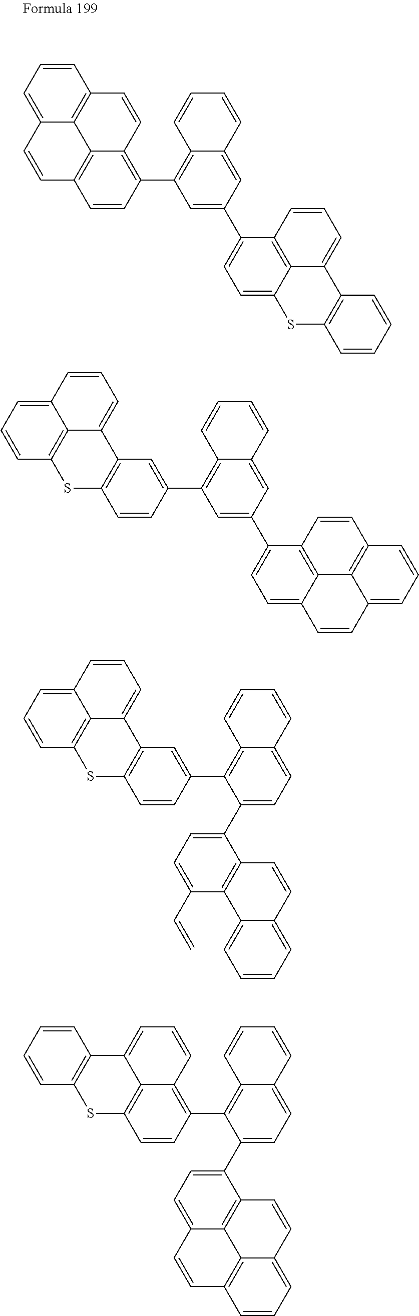 [SMILES: c1ccc2c(c1)Sc1ccc(-c3cc(-c4ccc5ccc6cccc7ccc4c5c67)c4ccccc4c3)c3cccc-2c13 ; c1ccc2c(-c3ccc4c(c3)-c3cccc5cccc(c35)S4)cc(-c3ccc4ccc5cccc6ccc3c4c56)cc2c1 ; C=Cc1ccc(-c2ccc3ccccc3c2-c2ccc3c(c2)-c2cccc4cccc(c24)S3)c2ccc3ccccc3c12 ; c1ccc2c(c1)Sc1ccc(-c3c(-c4ccc5ccc6cccc7ccc4c5c67)ccc4ccccc34)c3cccc-2c13]

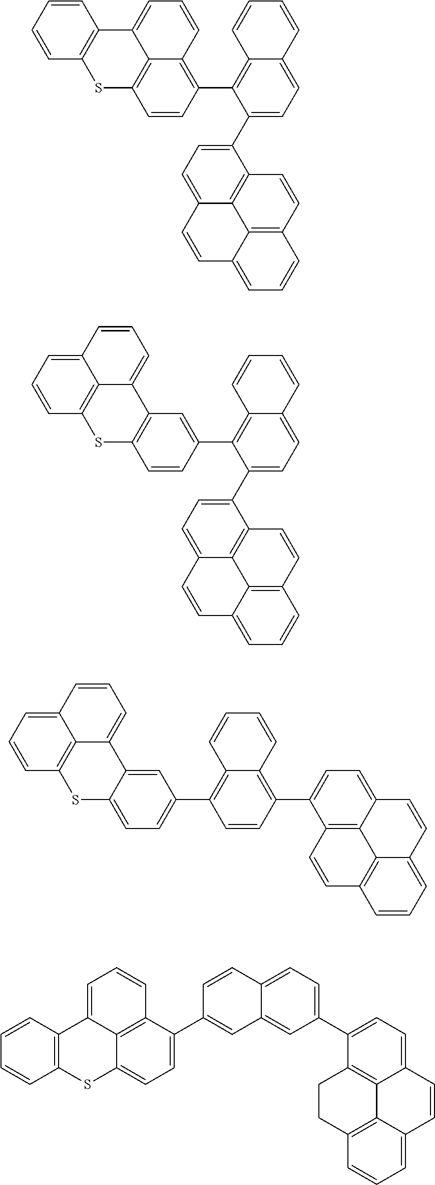 [SMILES: c1ccc2c(c1)Sc1ccc(-c3c(-c4ccc5ccc6cccc7ccc4c5c67)ccc4ccccc34)c3cccc-2c13 ; c1ccc2c(-c3ccc4c(c3)-c3cccc5cccc(c35)S4)c(-c3ccc4ccc5cccc6ccc3c4c56)ccc2c1 ; c1cc2c3c(cccc3c1)-c1cc(-c3ccc(-c4ccc5ccc6cccc7ccc4c5c67)c4ccccc34)ccc1S2 ; c1ccc2c(c1)Sc1ccc(-c3ccc4ccc(-c5ccc6ccc7cccc8c7c6c5CC8)cc4c3)c3cccc-2c13]

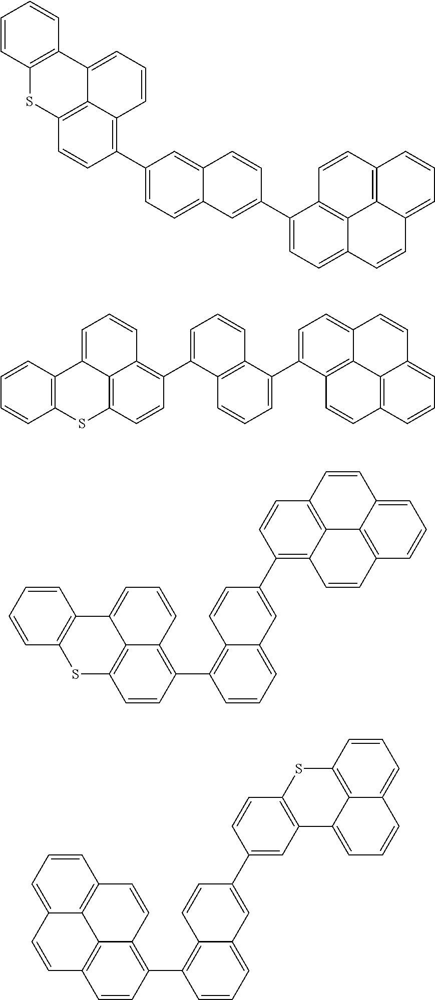

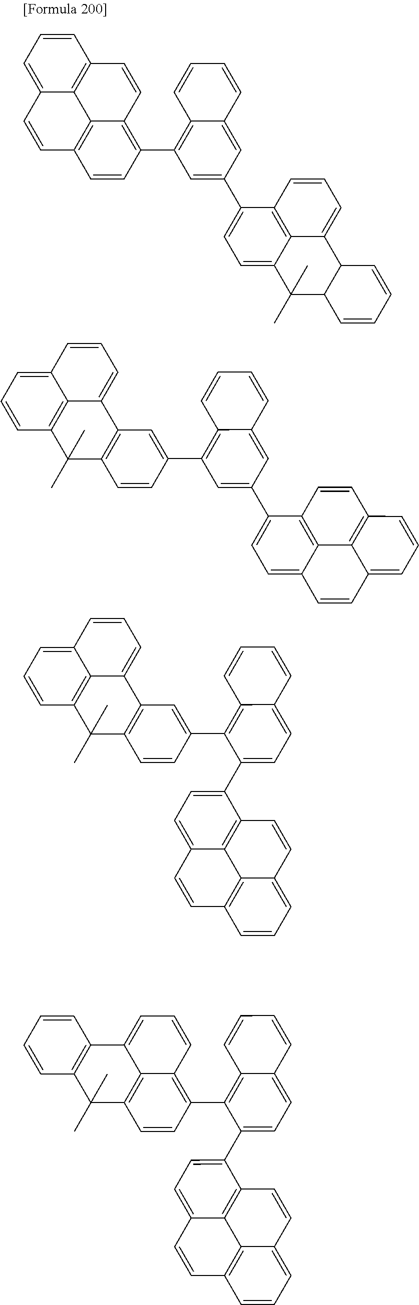

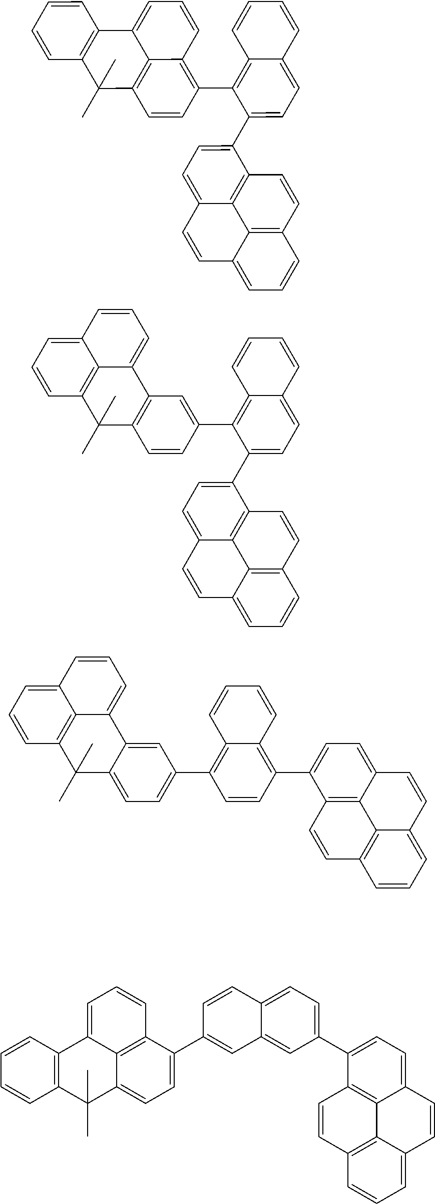

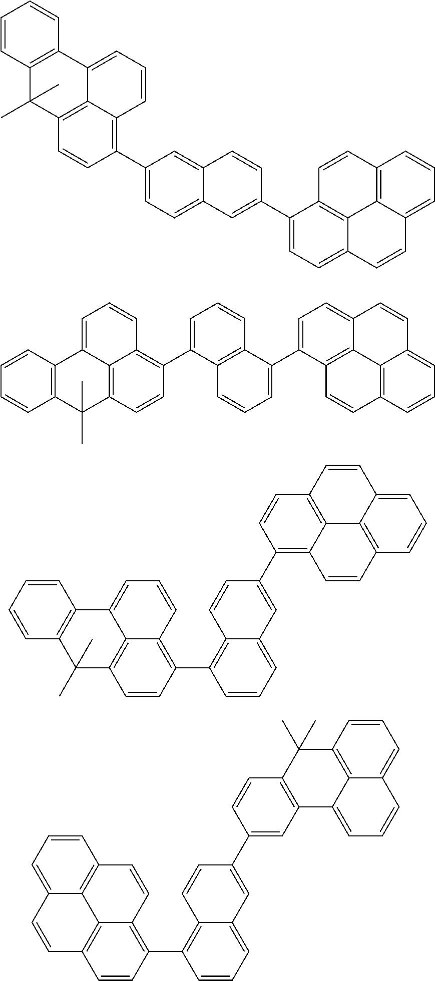

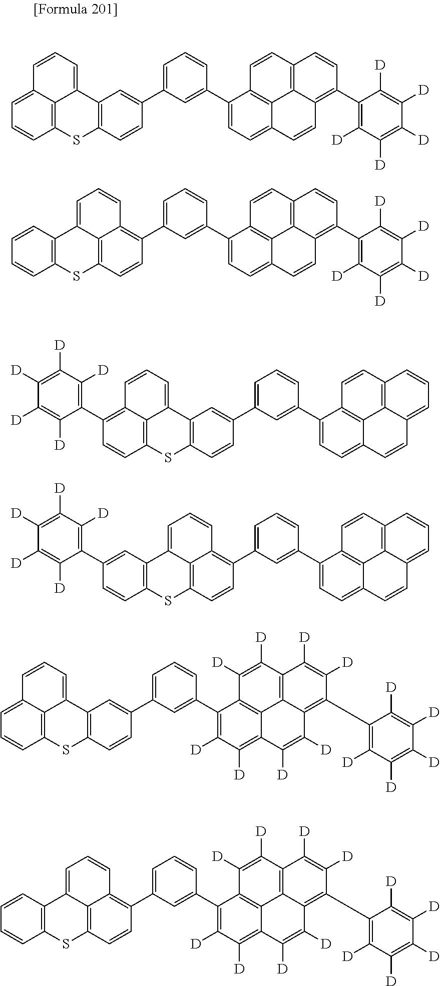

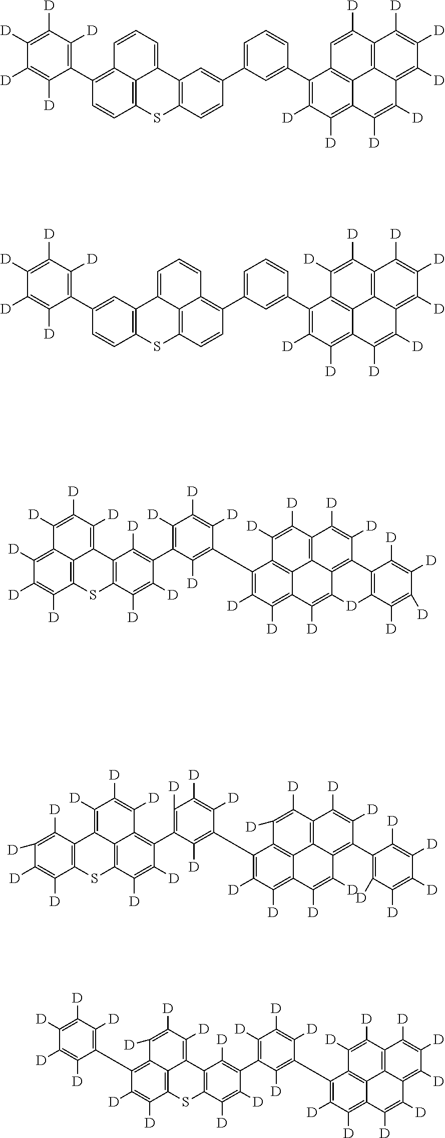

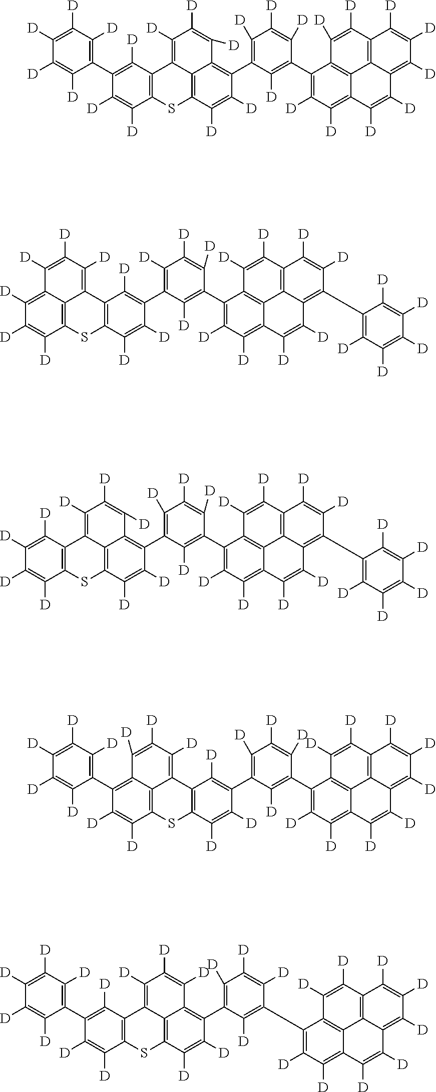

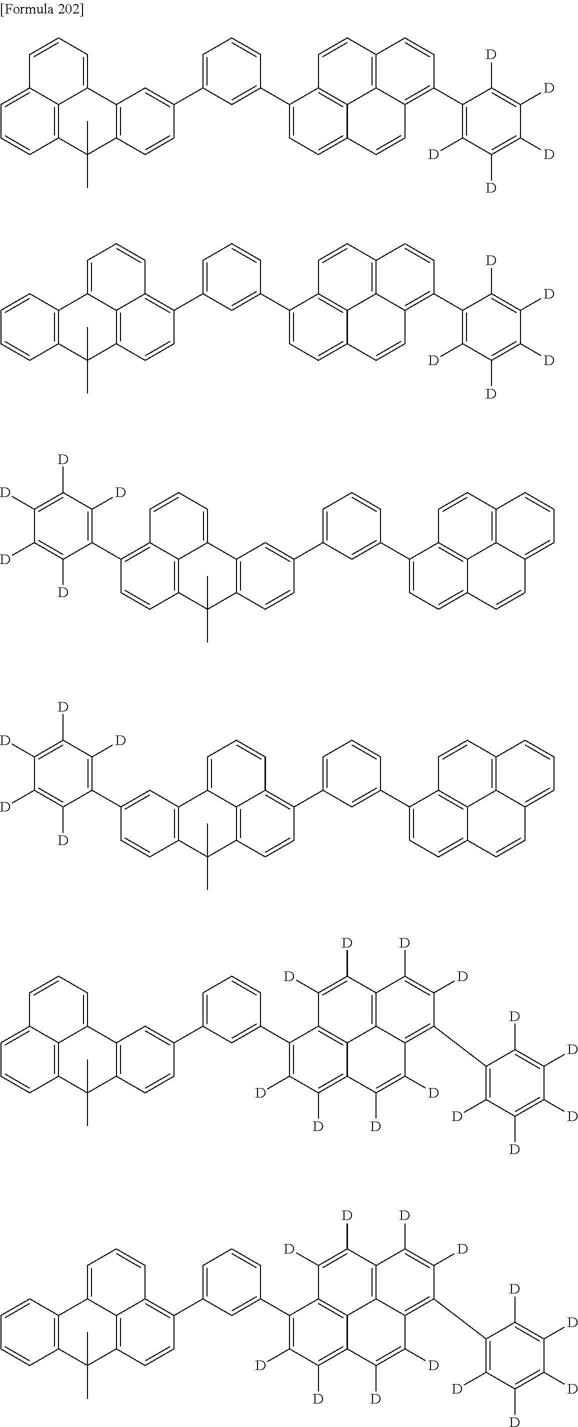

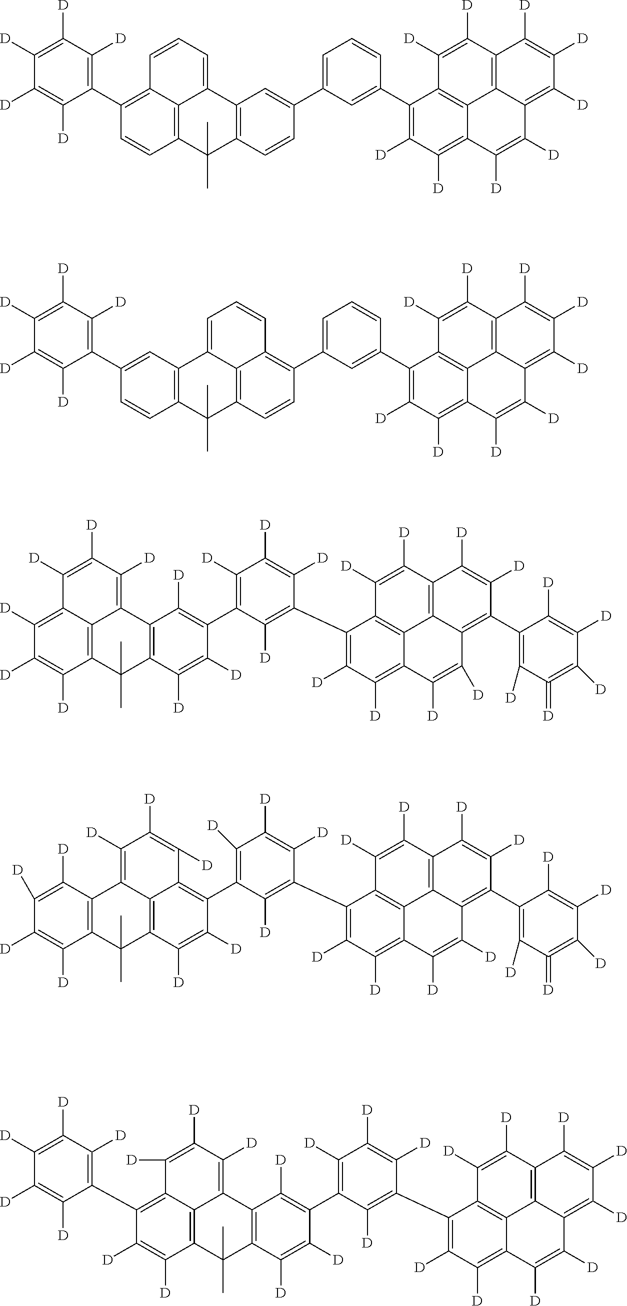

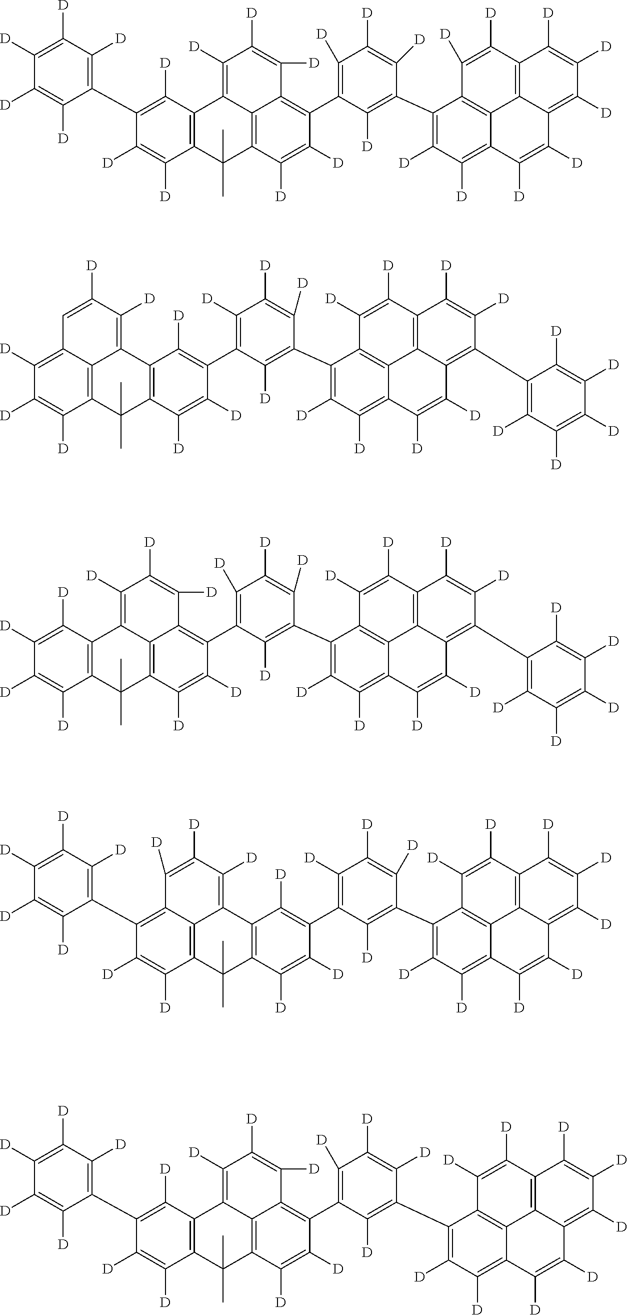

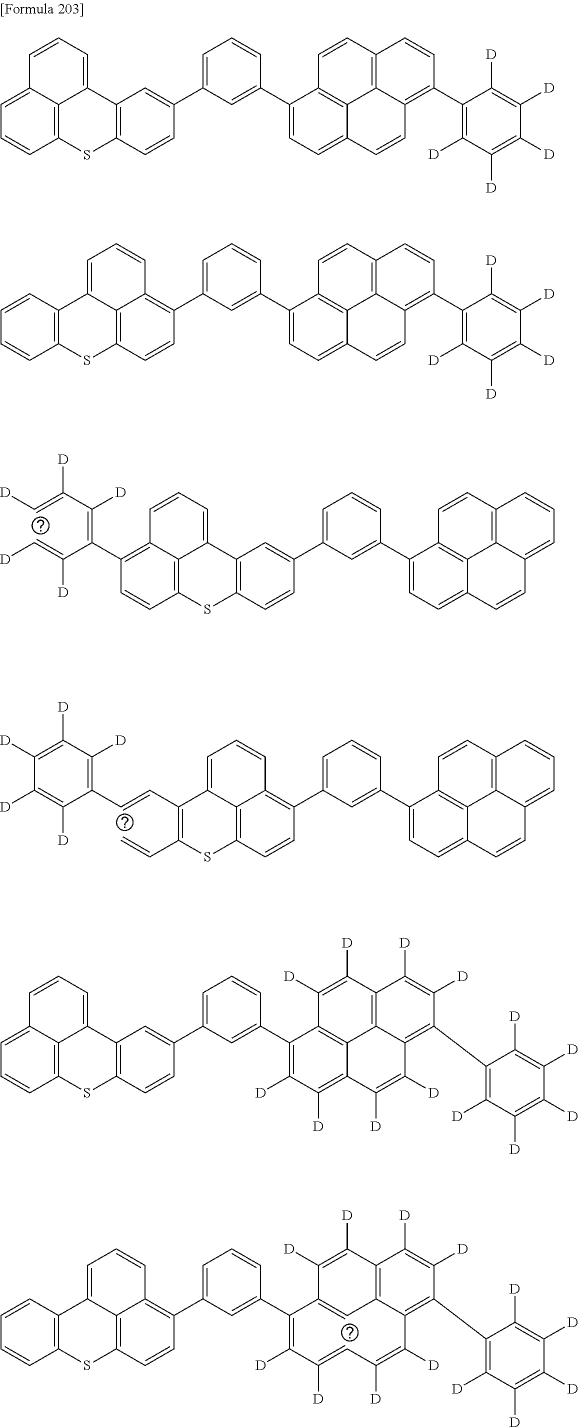

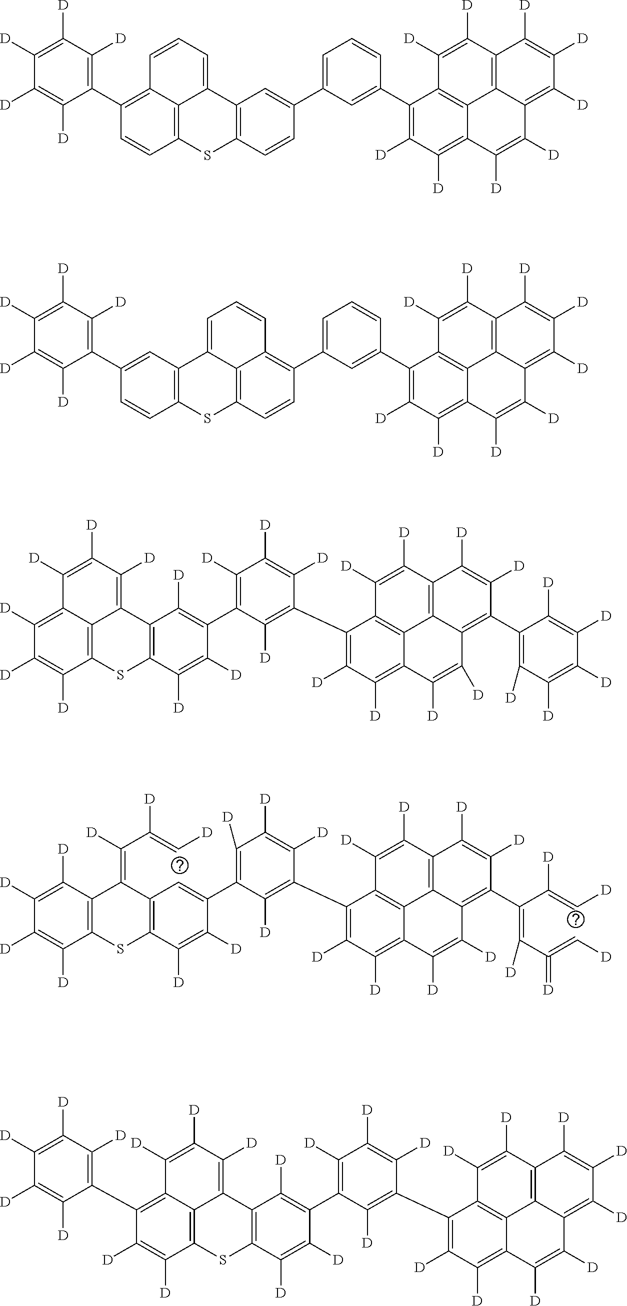

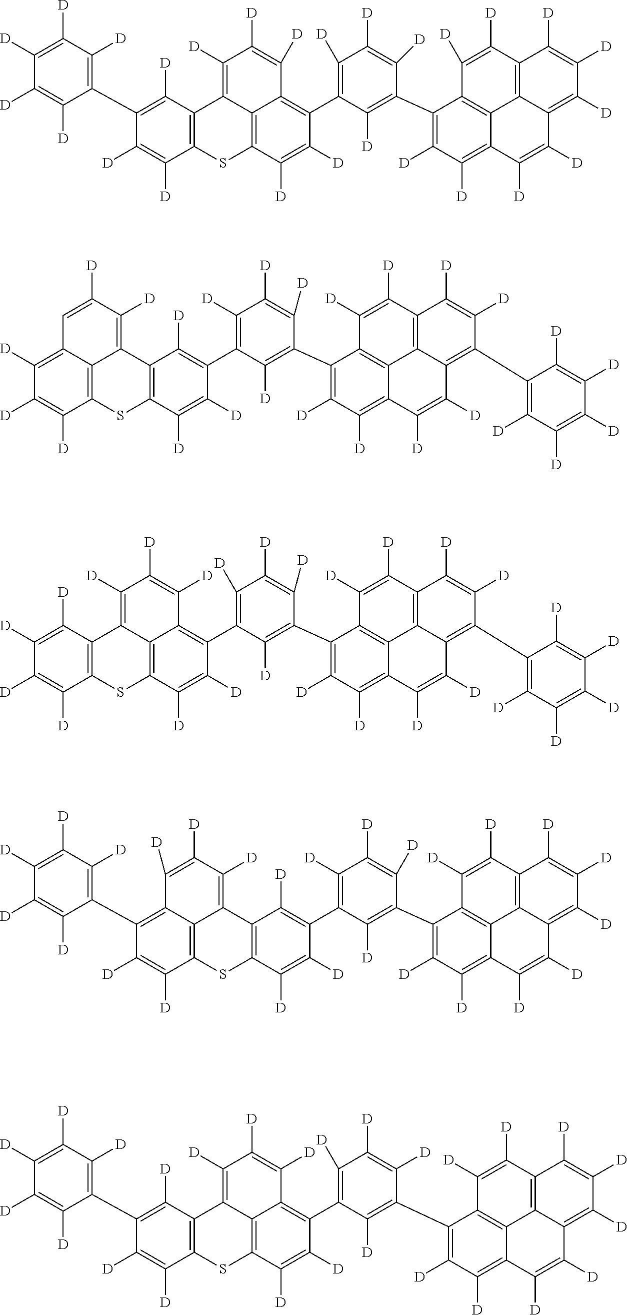

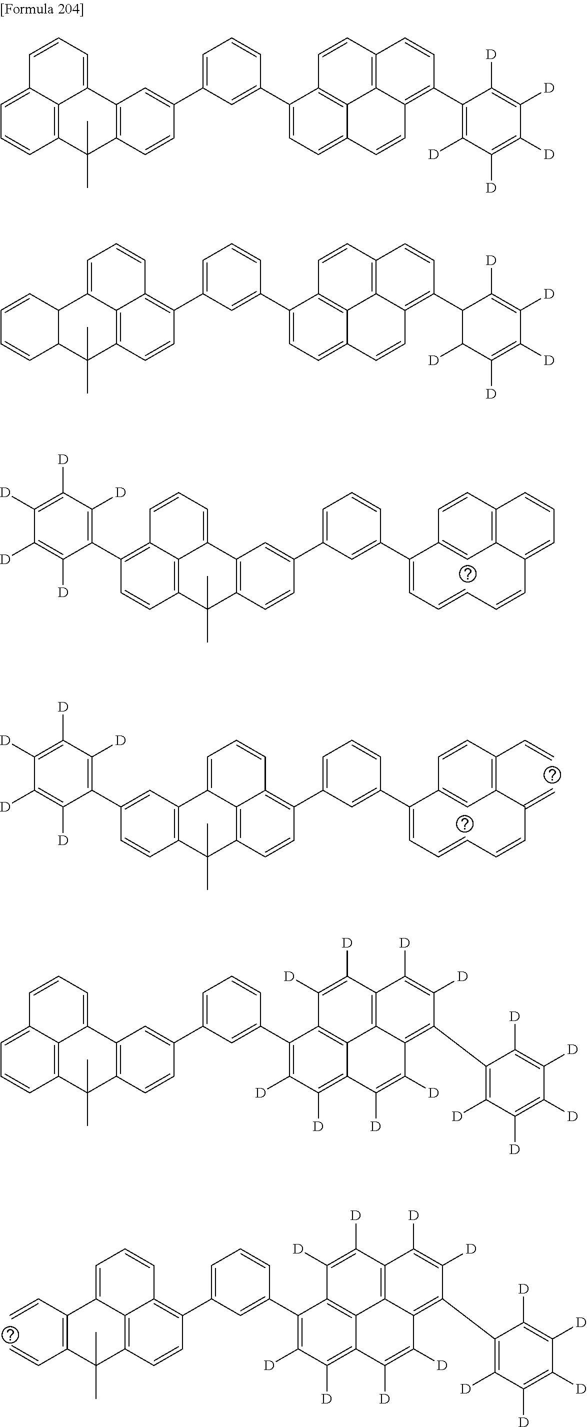

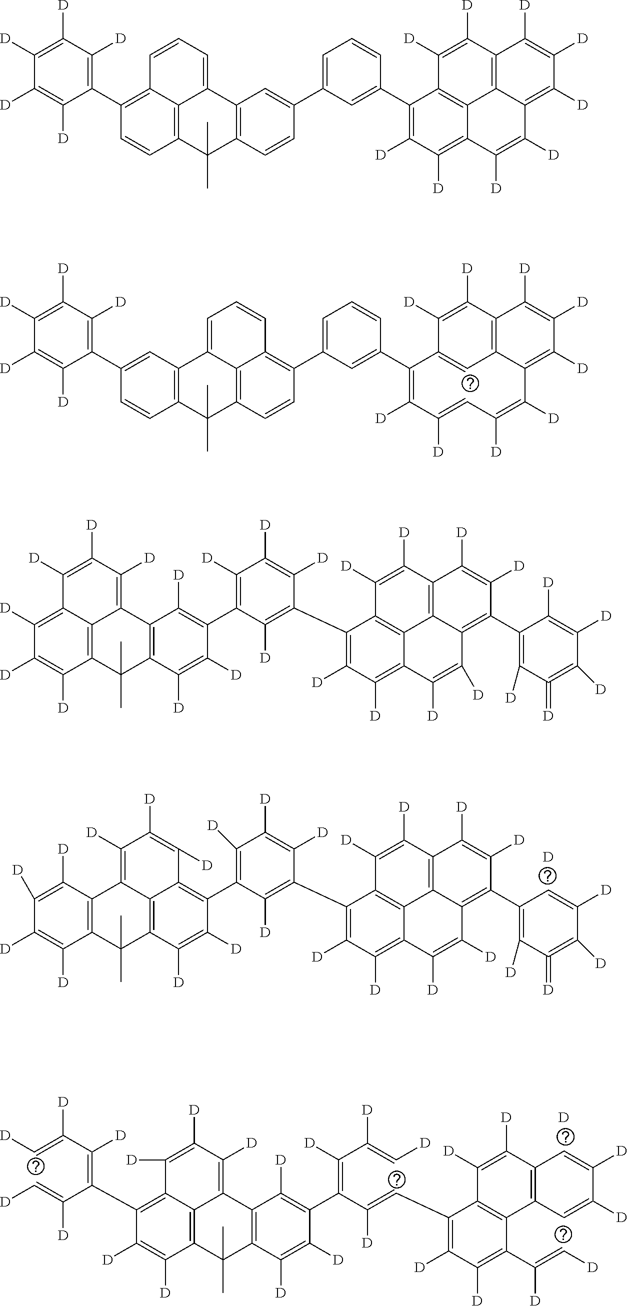

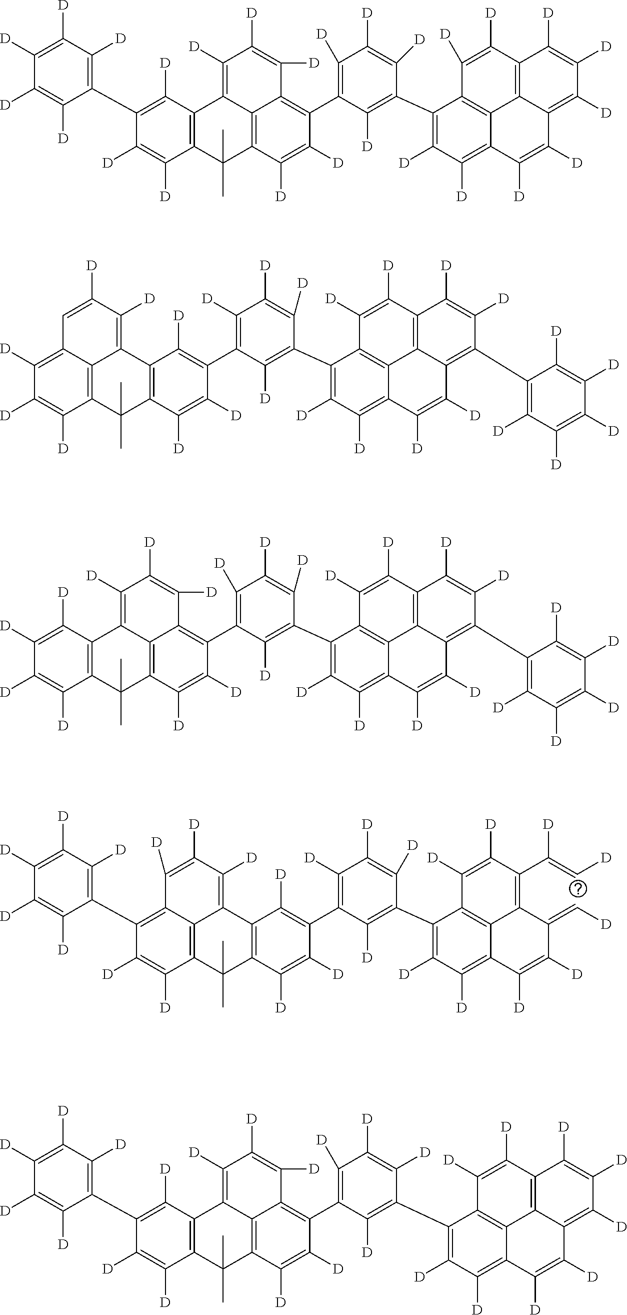

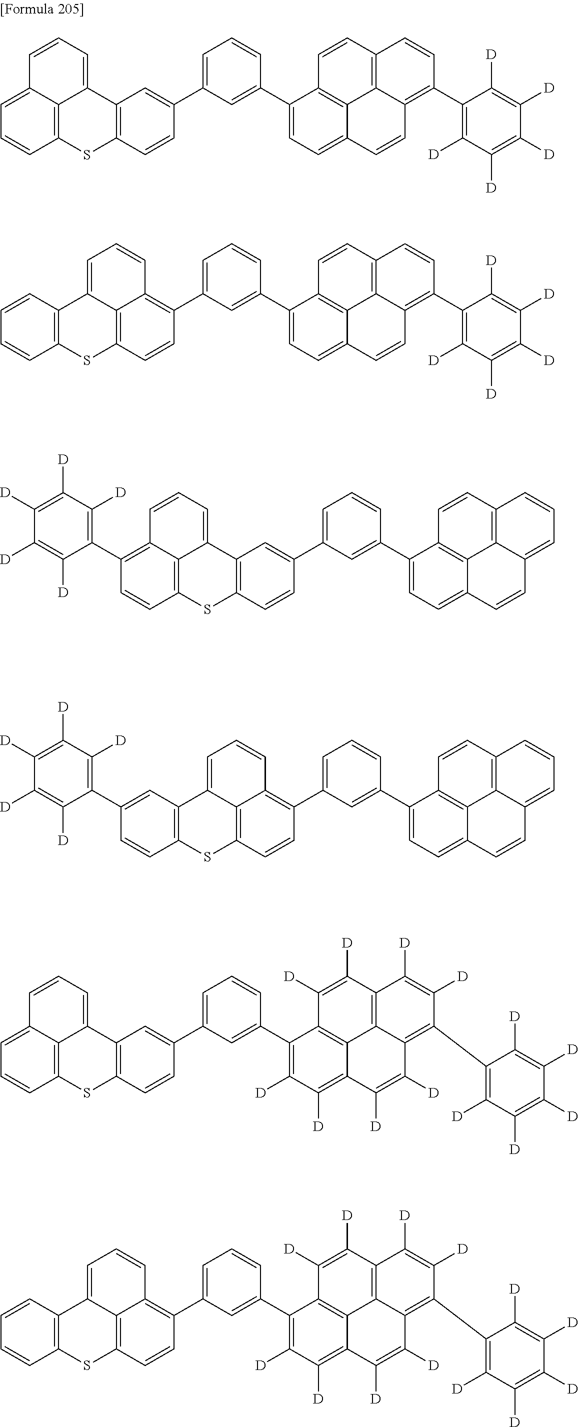

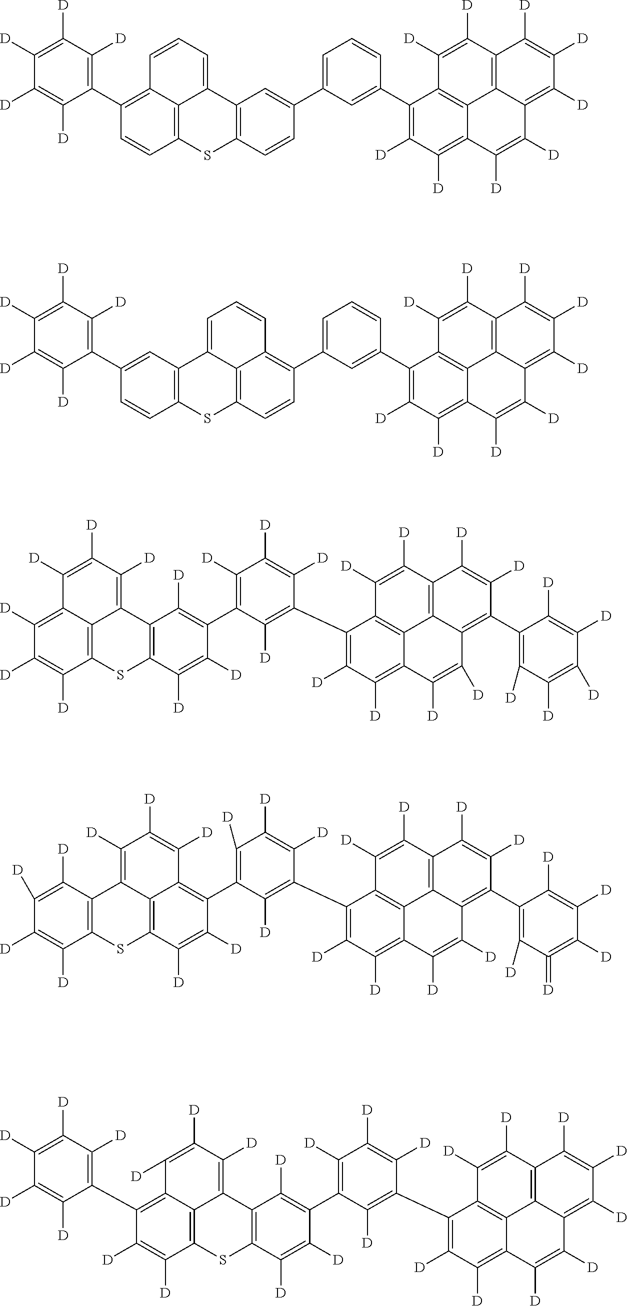

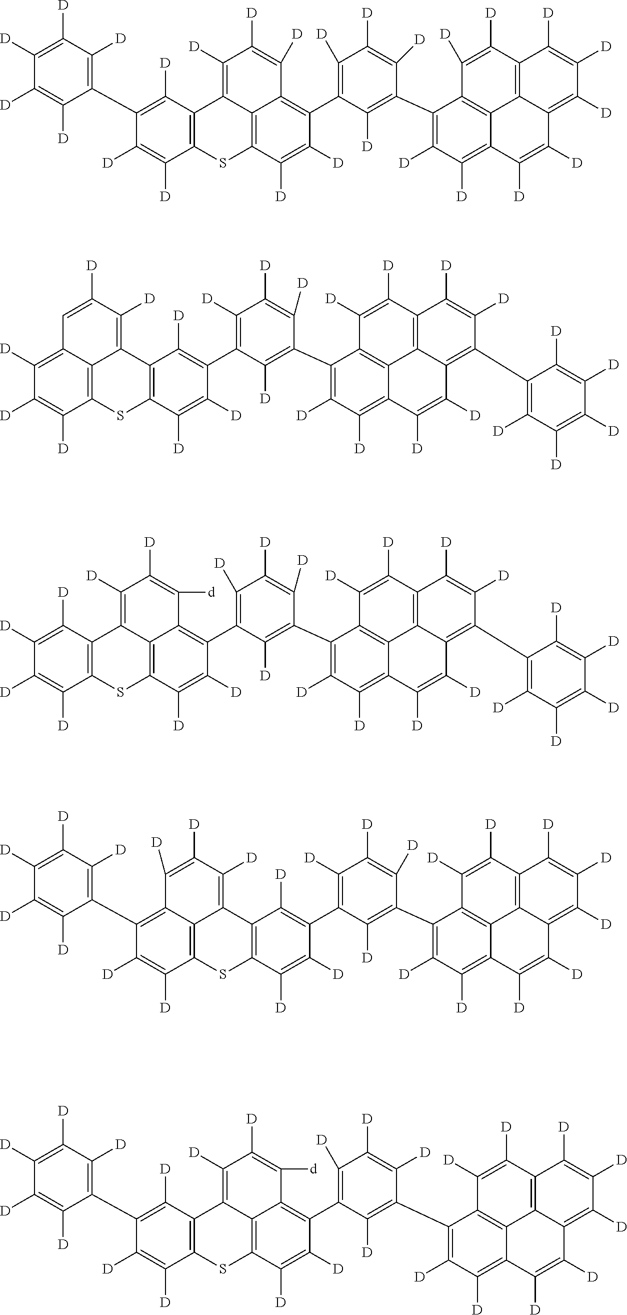

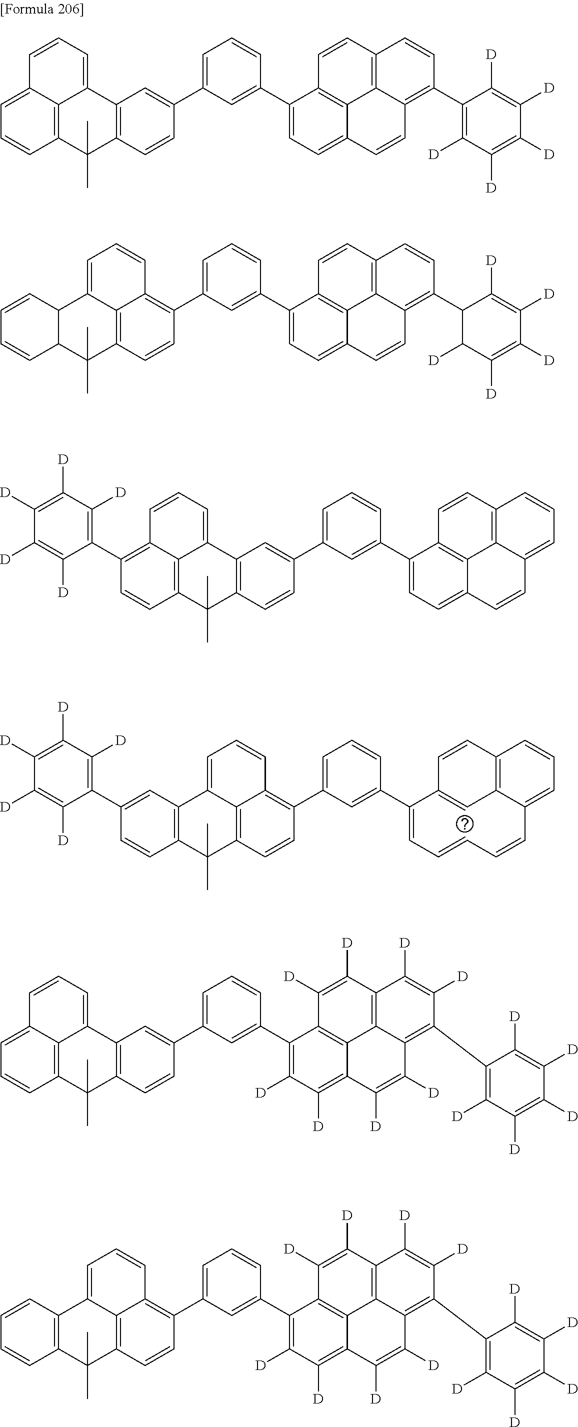

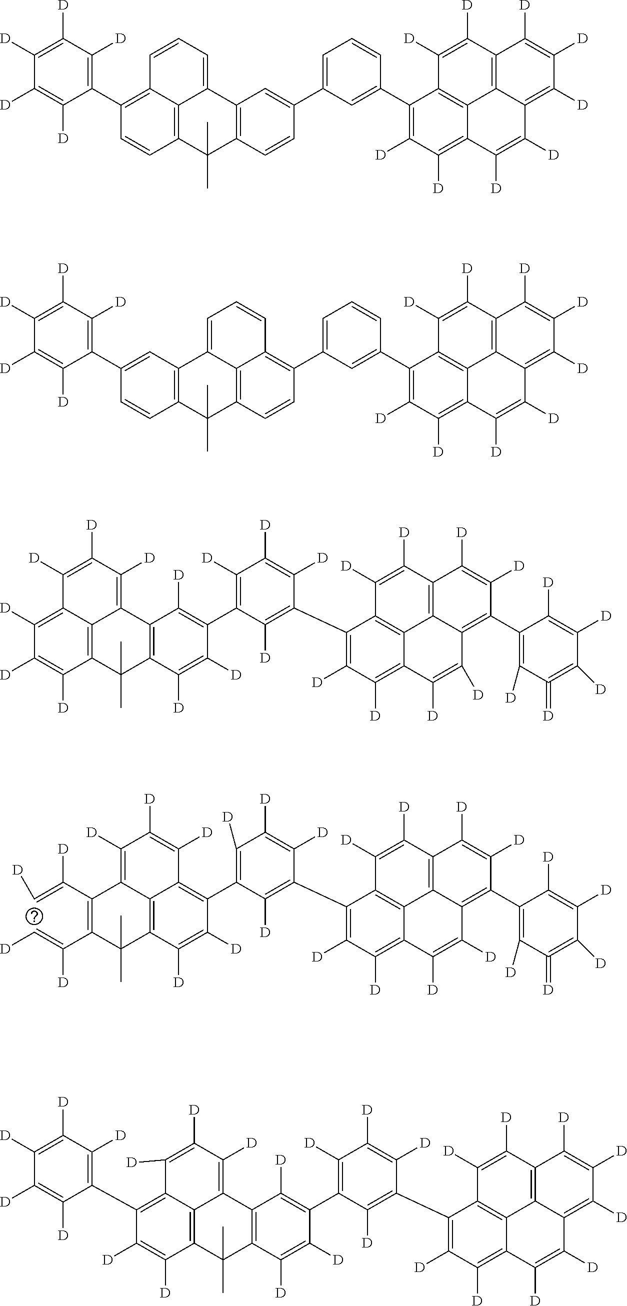

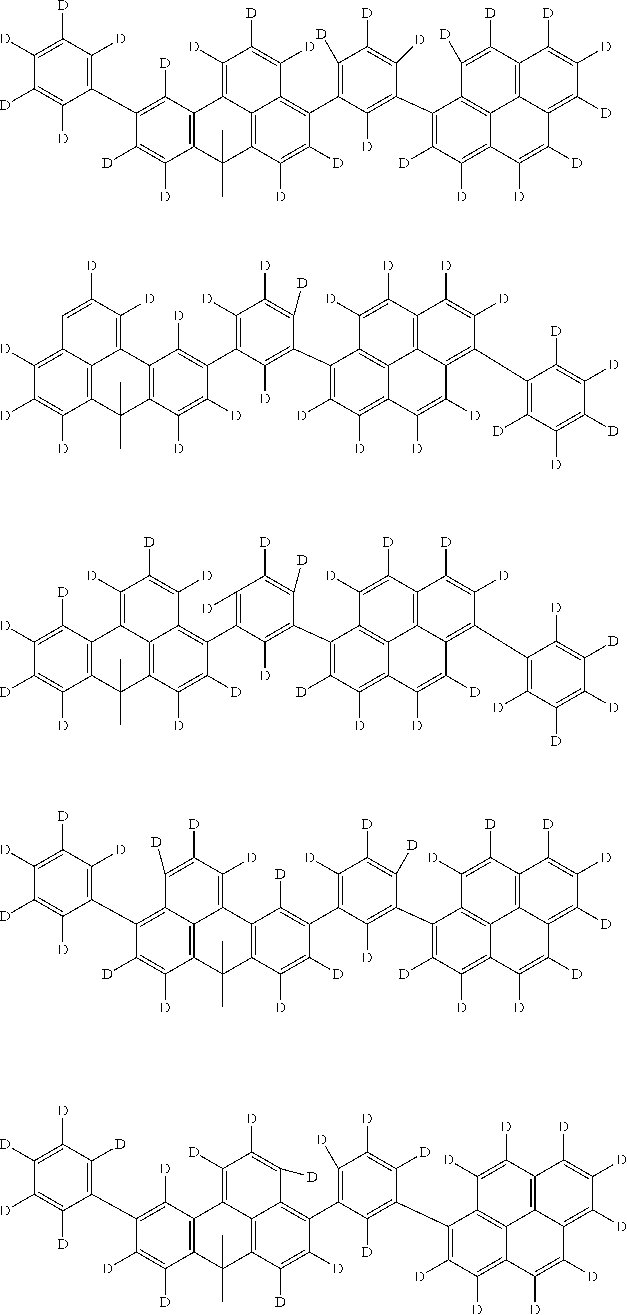

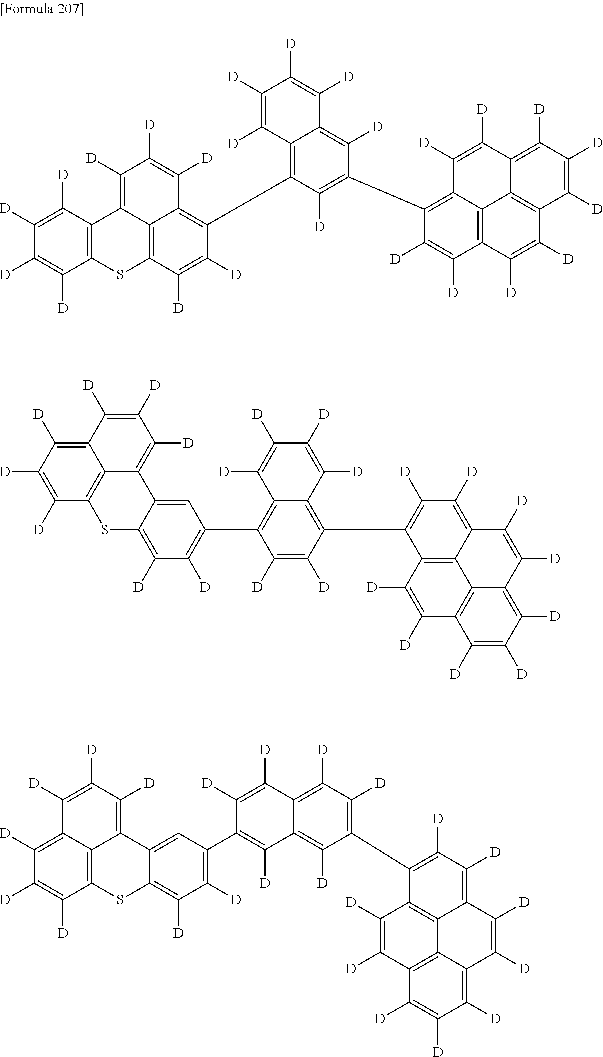

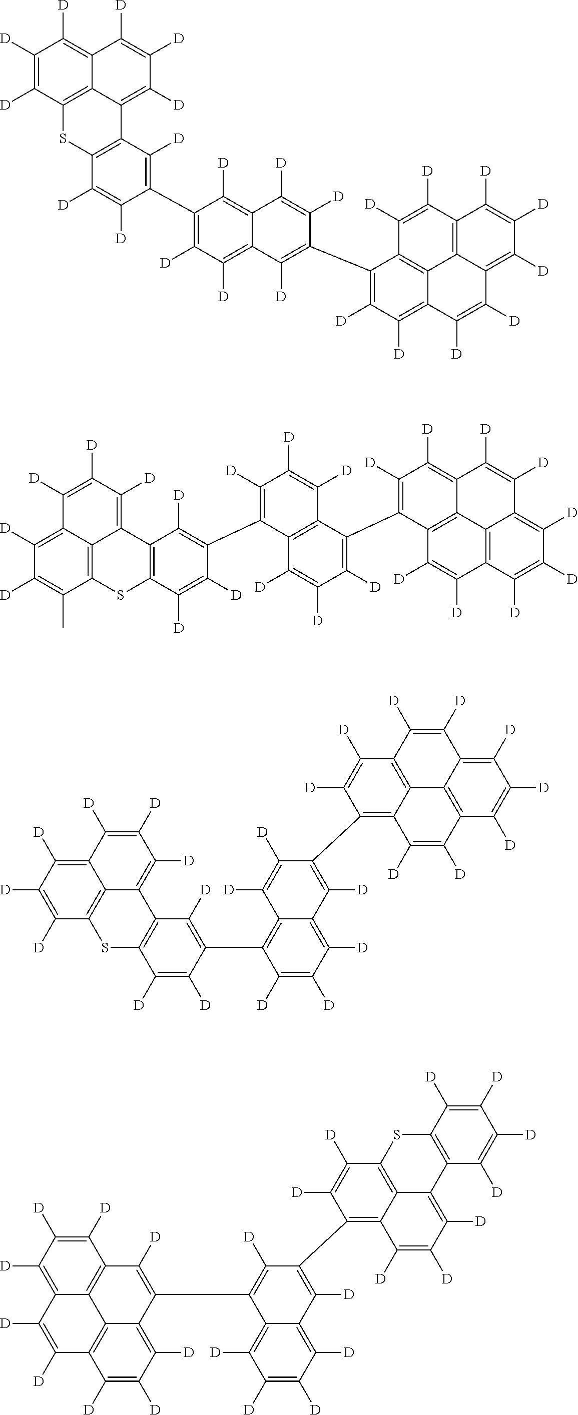

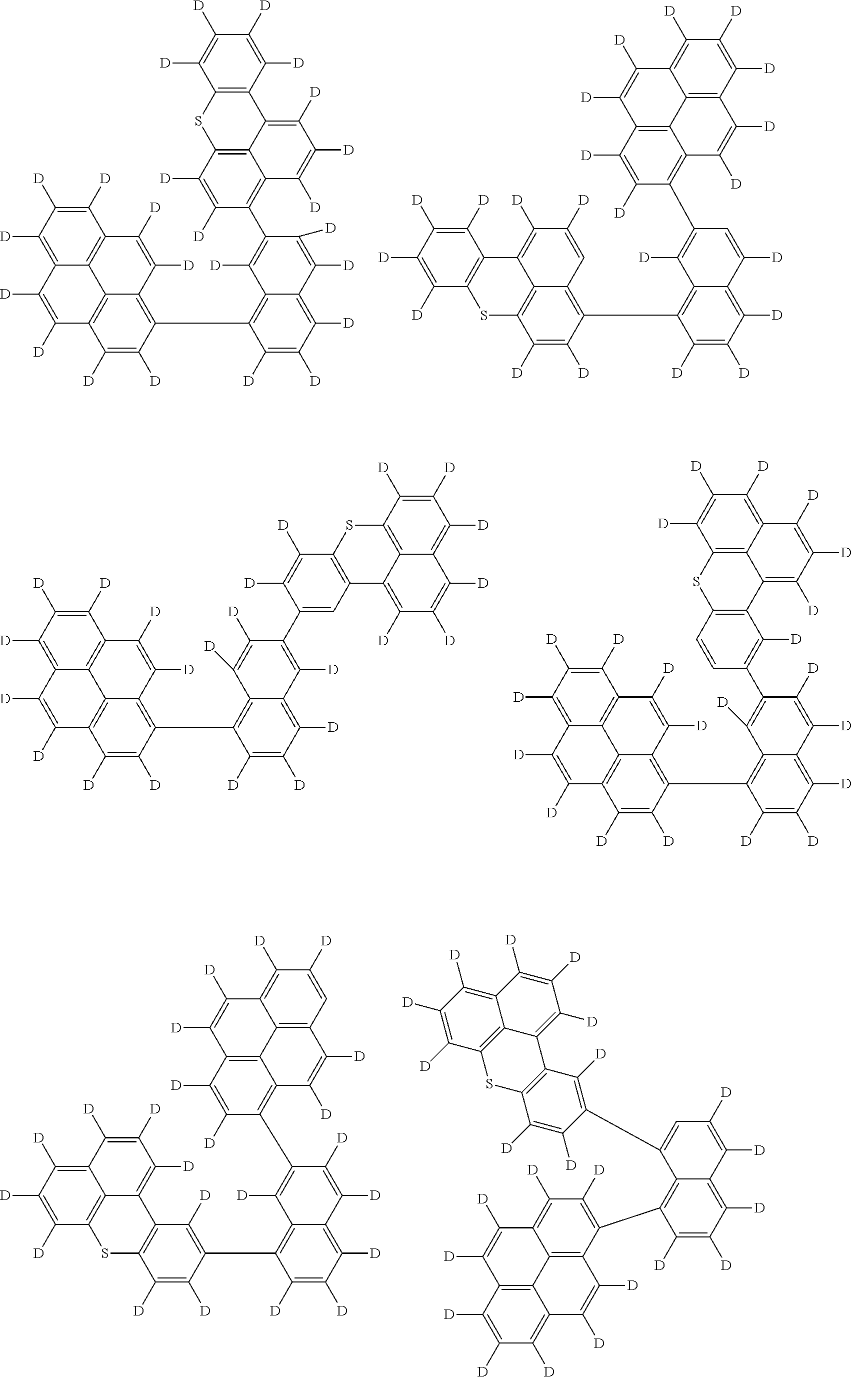

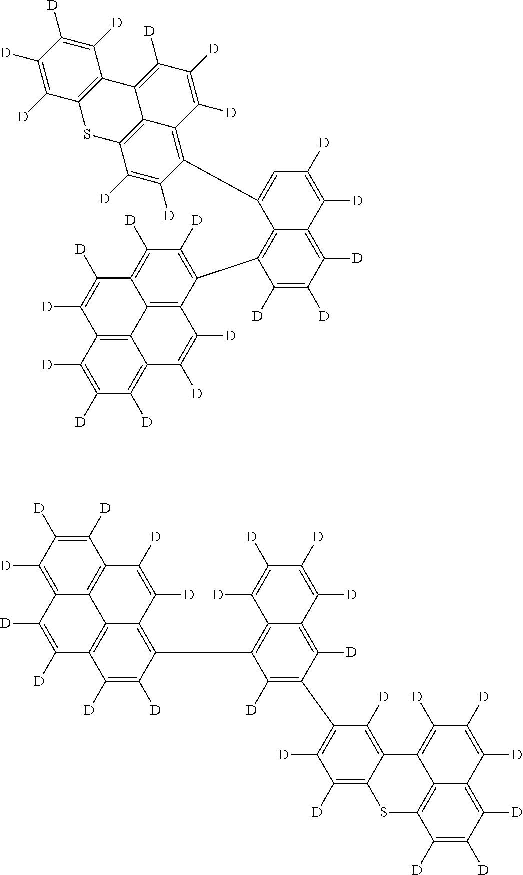 [SMILES: [2H]c1cc(-c2c([2H])c([2H])c3c4c(c([2H])c([2H])c([2H])c24)-c2c([2H])c([2H])c([2H])c([2H])c2S3)c2c(-c3c([2H])c([2H])c4c([2H])c([2H])c5c([2H])c([2H])c([2H])c6c([2H])c([2H])c3c4c56)c([2H])c([2H])c([2H])c2c1[2H] ; [2H]c1c([2H])c(-c2c([2H])c(-c3c([2H])c([2H])c4c([2H])c([2H])c5c([2H])c([2H])c([2H])c6c([2H])c([2H])c3c4c56)c3c([2H])c([2H])c([2H])c([2H])c3c2[2H])c([2H])c2c1Sc1c([2H])c([2H])c([2H])c3c([2H])c([2H])c([2H])c-2c13]

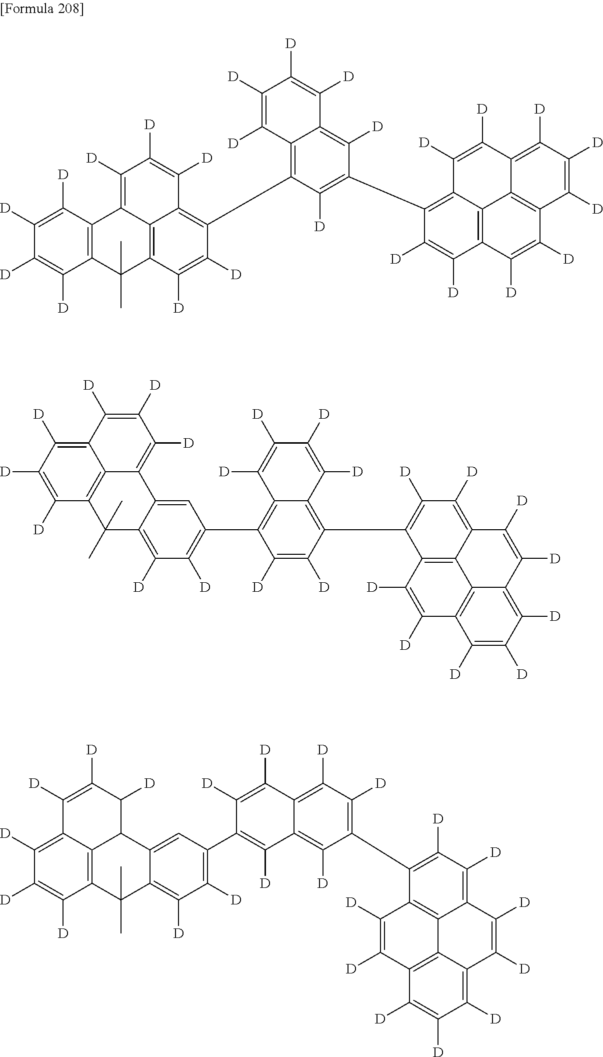

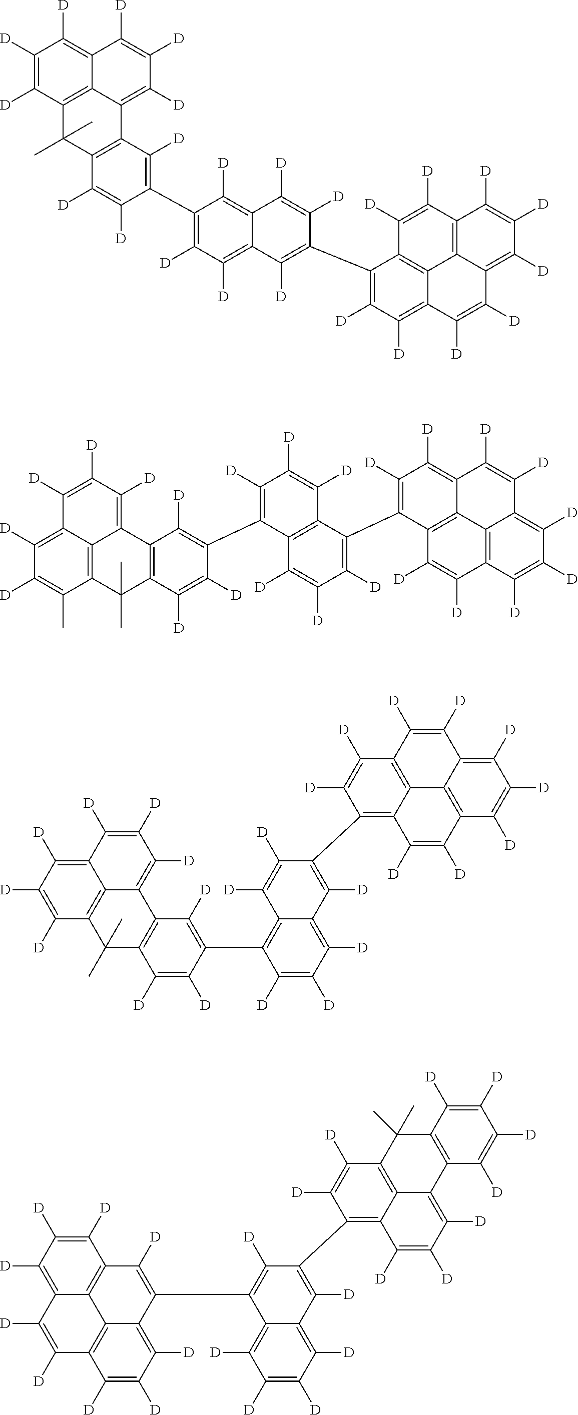

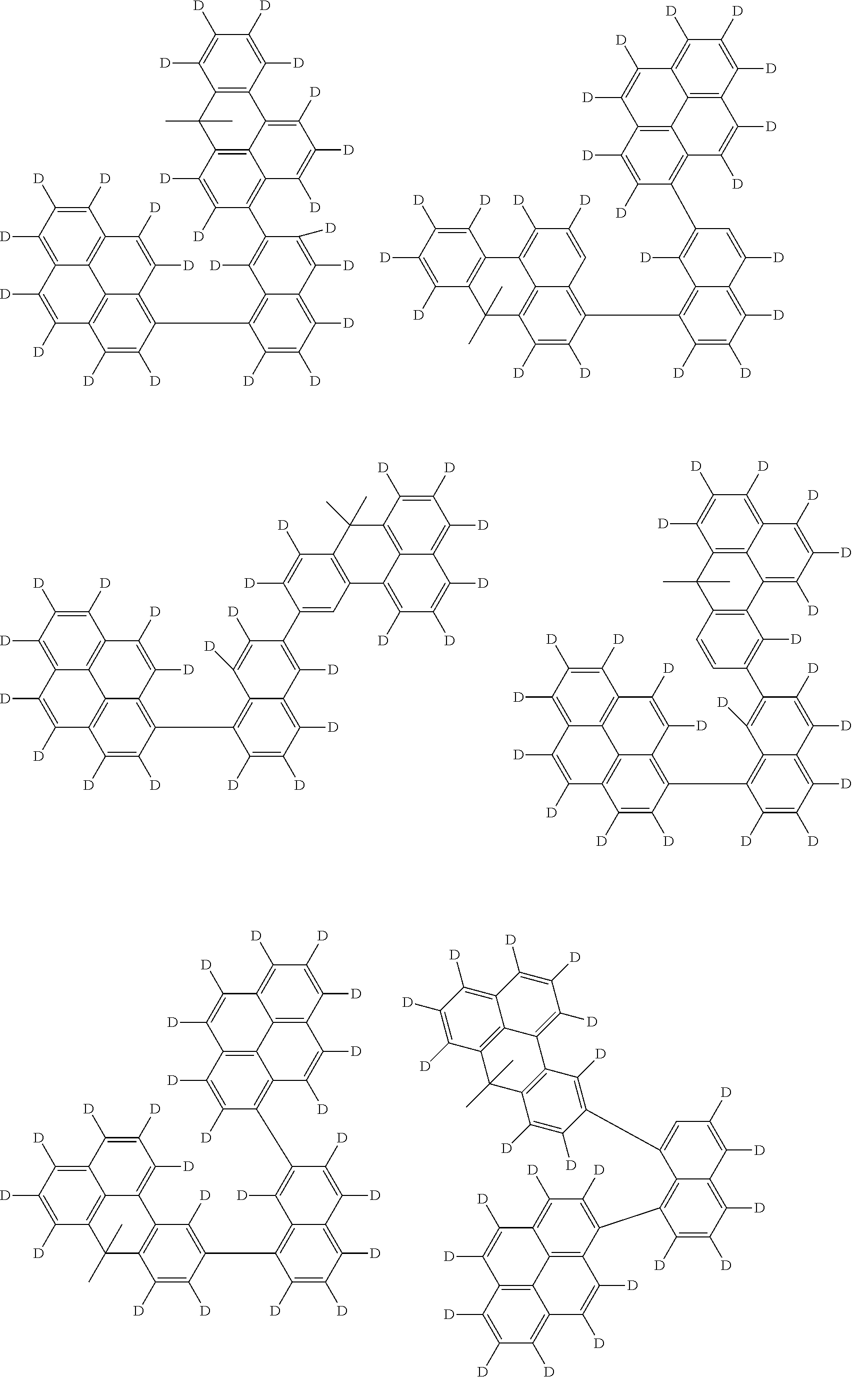

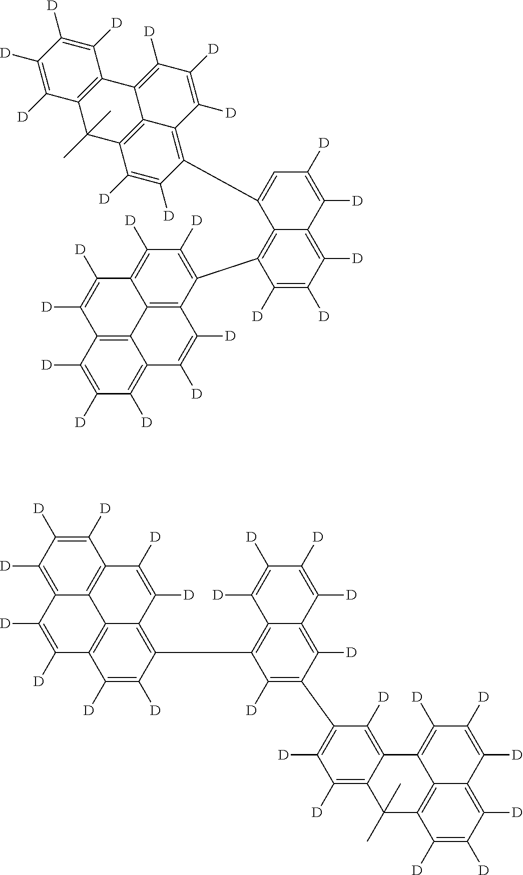

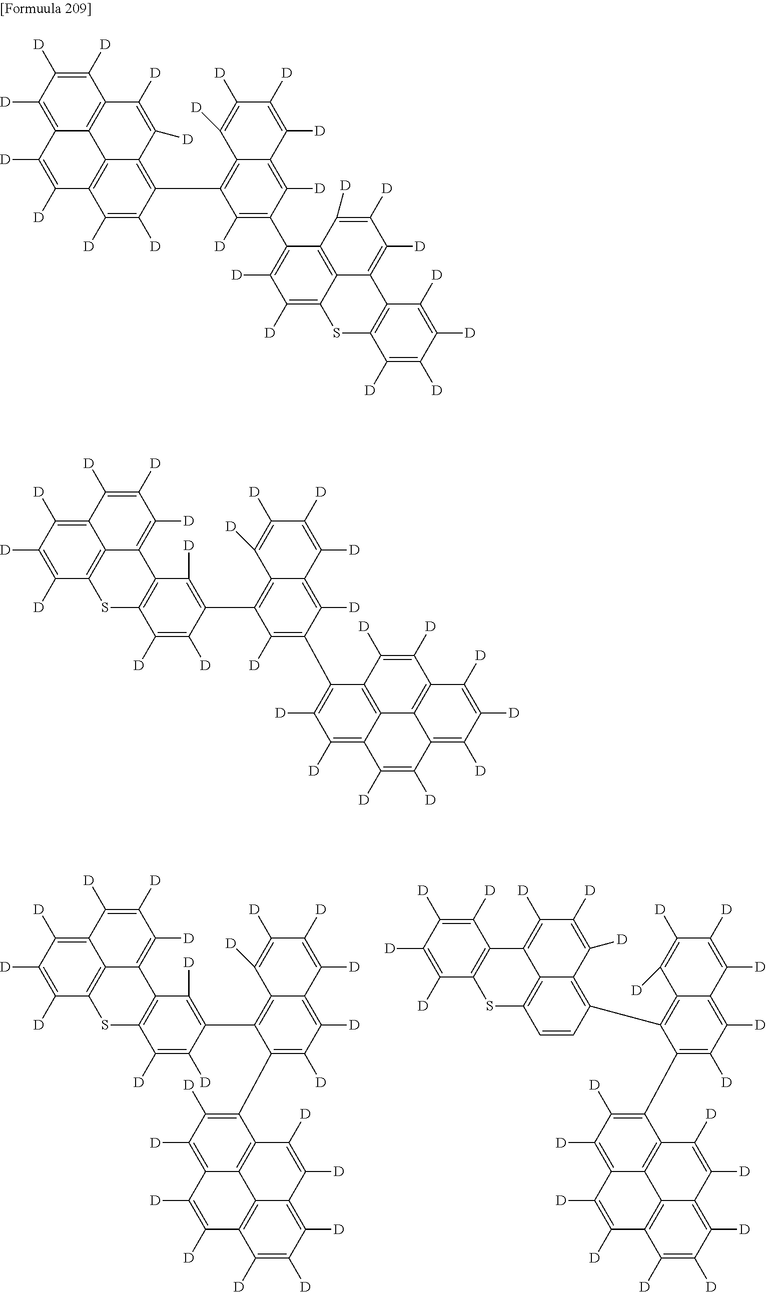

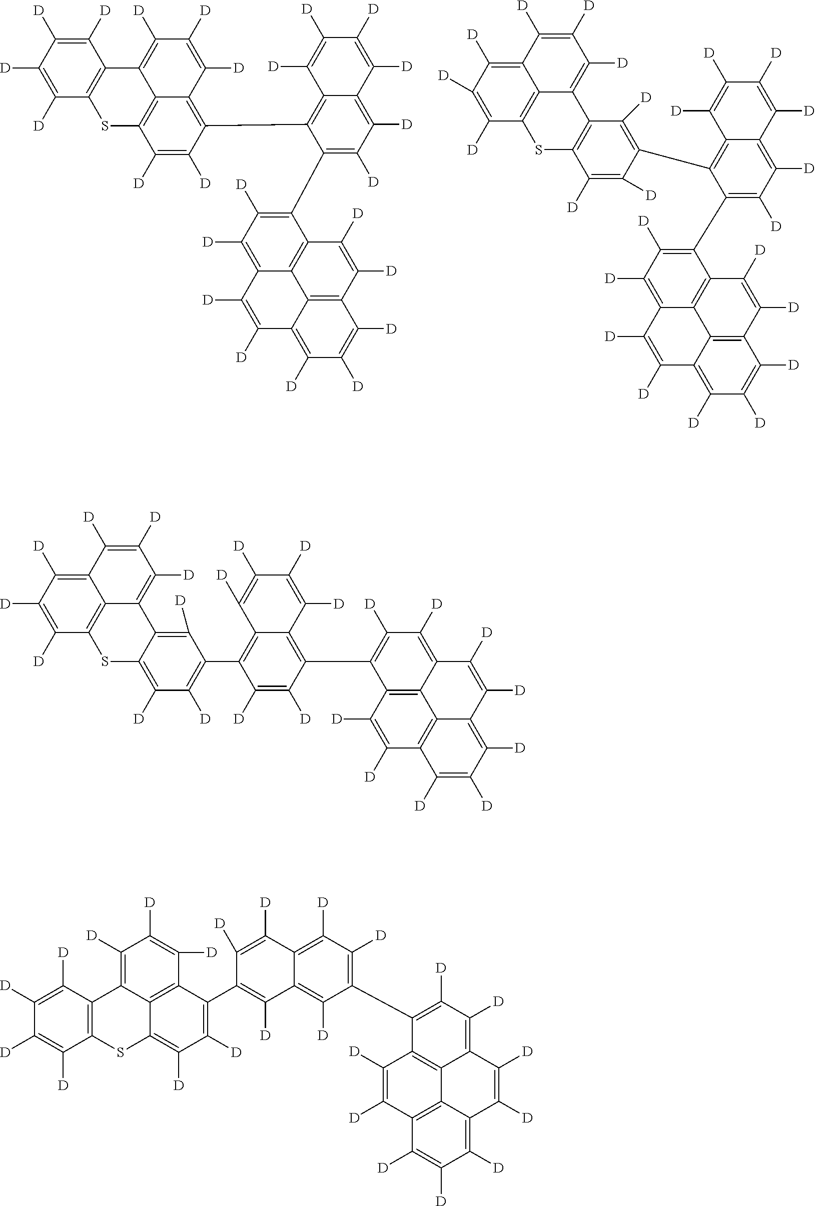

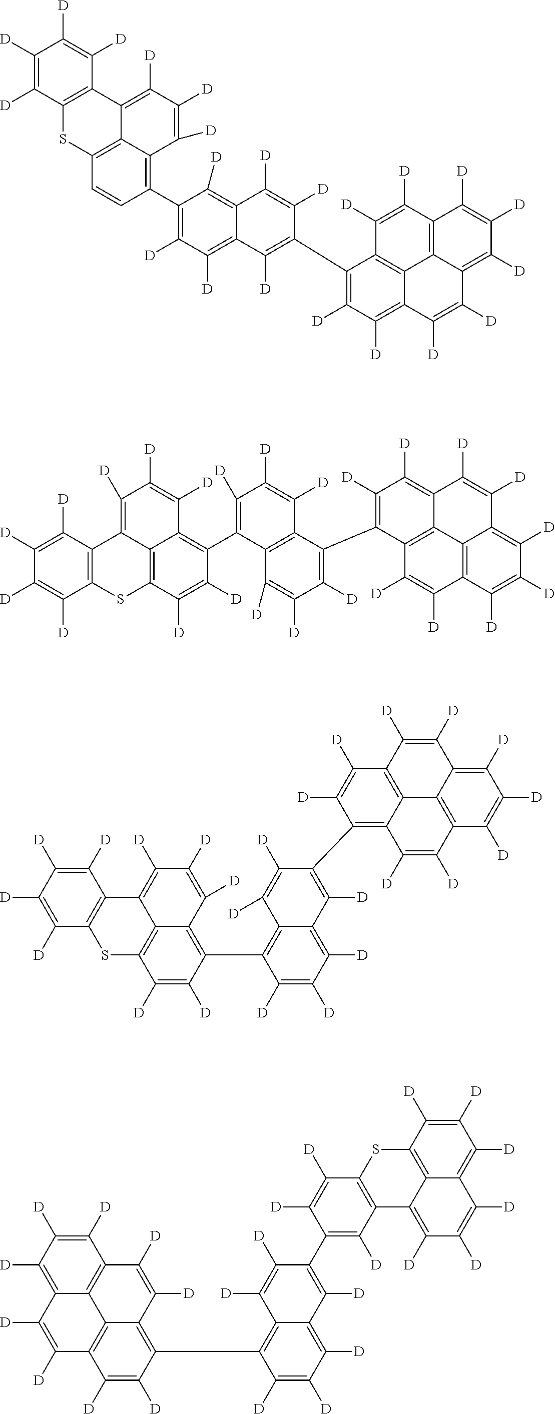

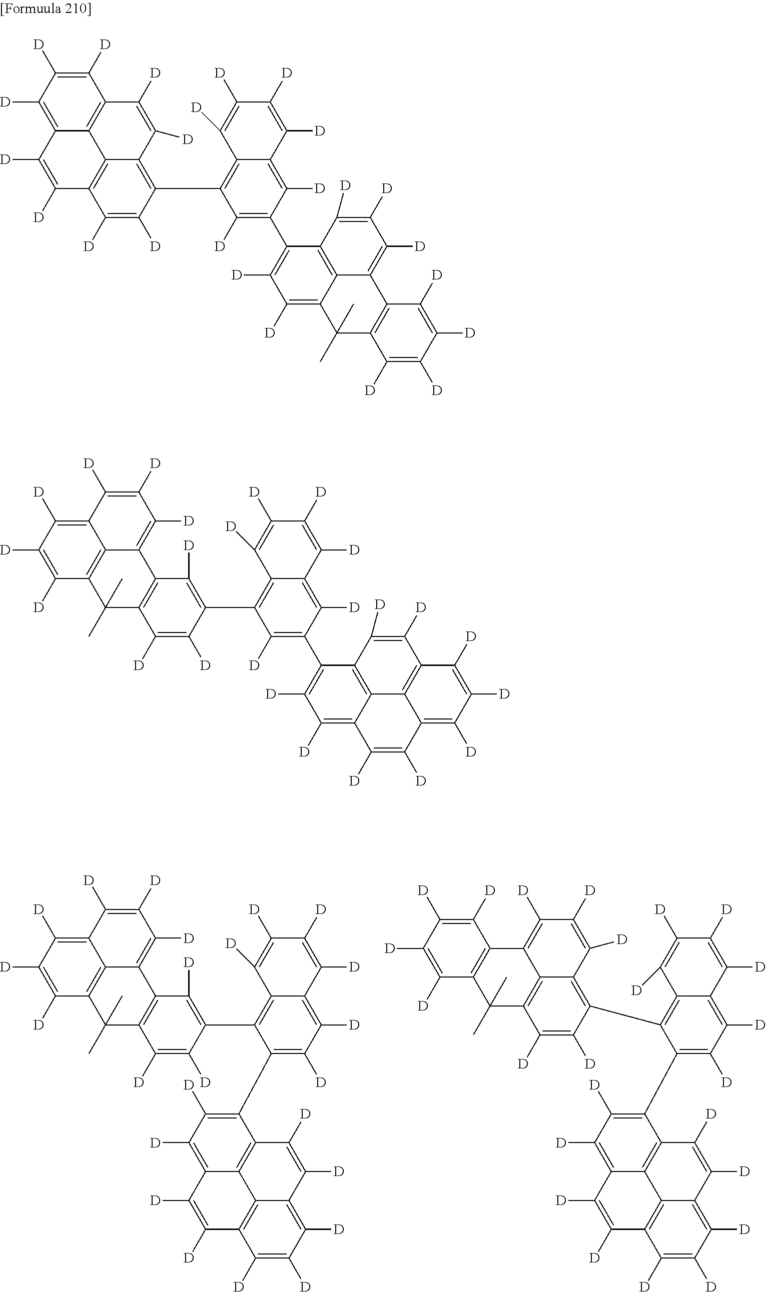

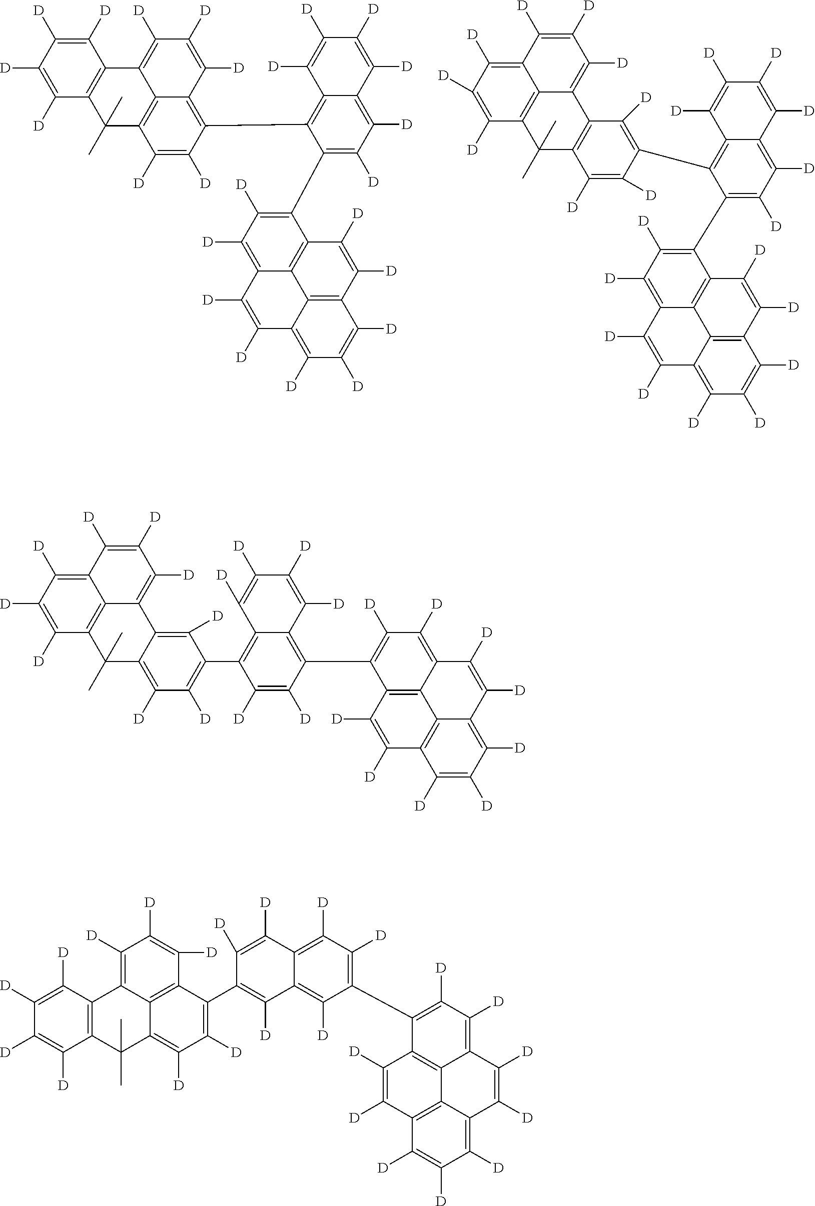

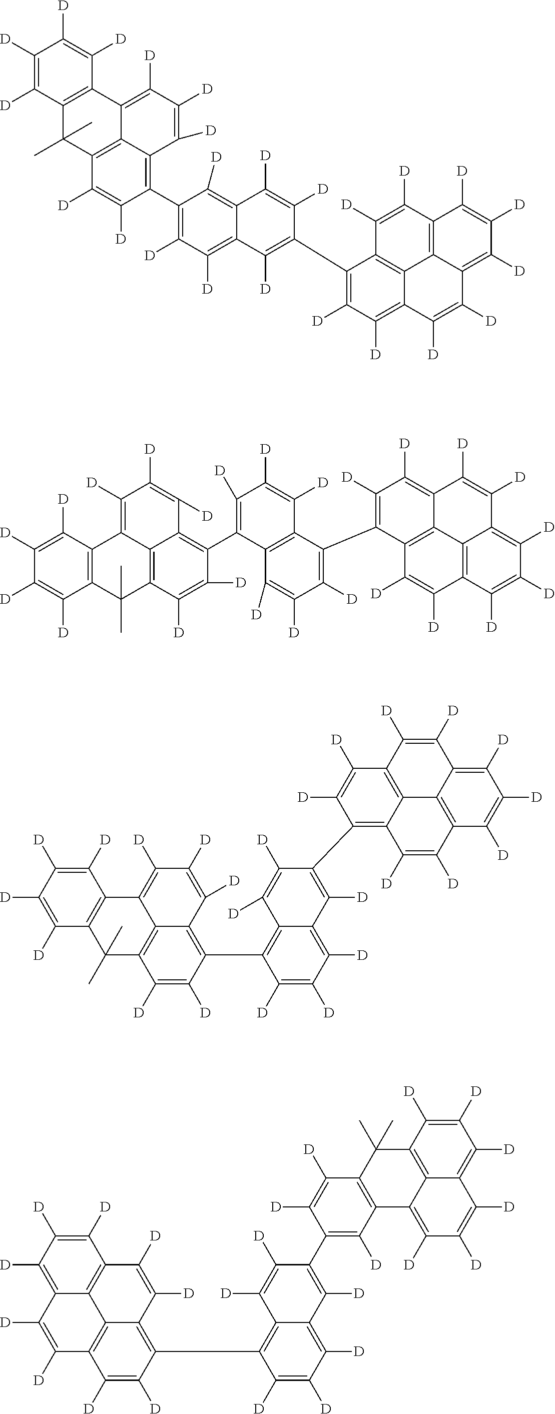

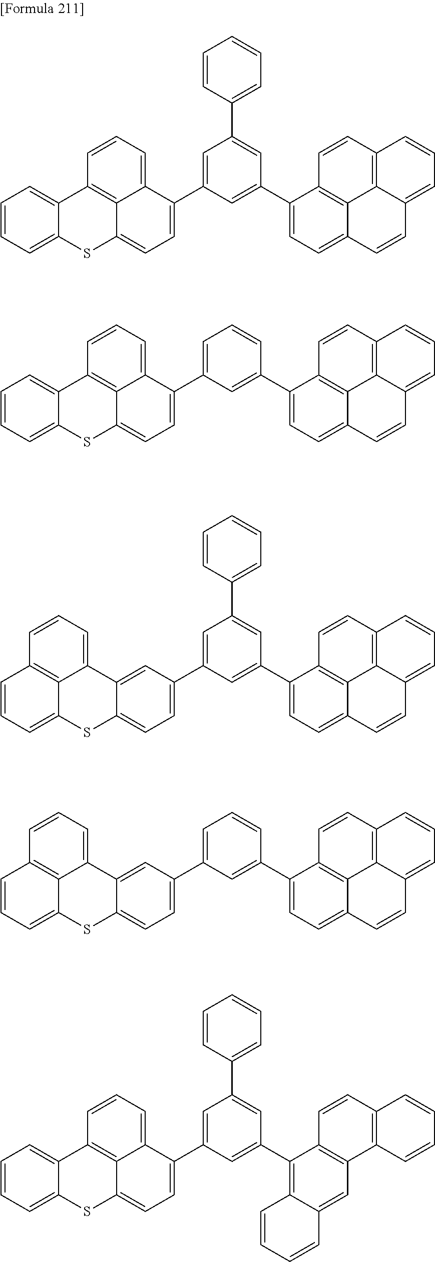 [SMILES: c1ccc(-c2cc(-c3ccc4c5c(cccc35)-c3ccccc3S4)cc(-c3ccc4ccc5cccc6ccc3c4c56)c2)cc1 ; c1cc(-c2ccc3c4c(cccc24)-c2ccccc2S3)cc(-c2ccc3ccc4cccc5ccc2c3c45)c1 ; c1ccc(-c2cc(-c3ccc4c(c3)-c3cccc5cccc(c35)S4)cc(-c3ccc4ccc5cccc6ccc3c4c56)c2)cc1 ; c1cc(-c2ccc3c(c2)-c2cccc4cccc(c24)S3)cc(-c2ccc3ccc4cccc5ccc2c3c45)c1 ; c1ccc(-c2cc(-c3ccc4c5c(cccc35)-c3ccccc3S4)cc(-c3c4ccccc4cc4c3ccc3ccccc34)c2)cc1]

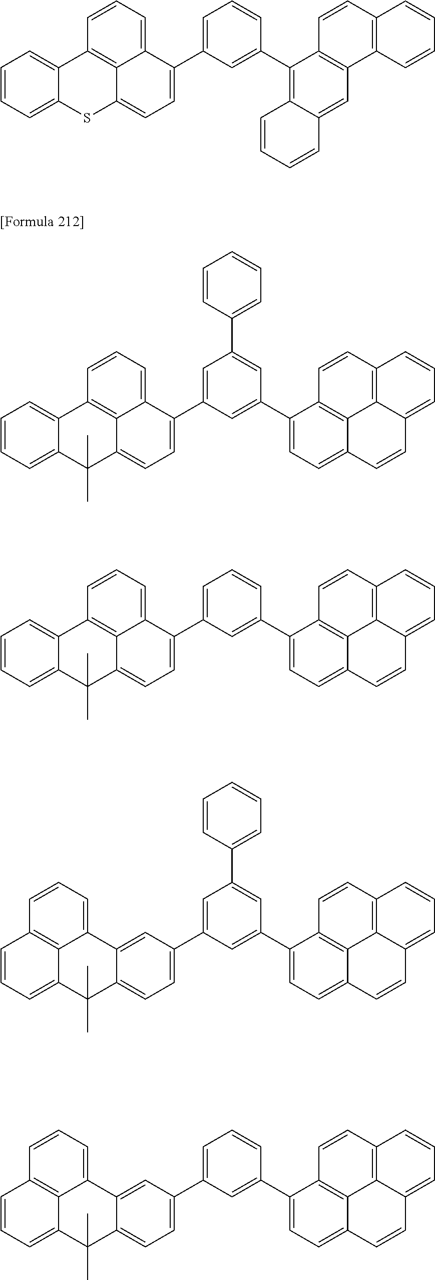

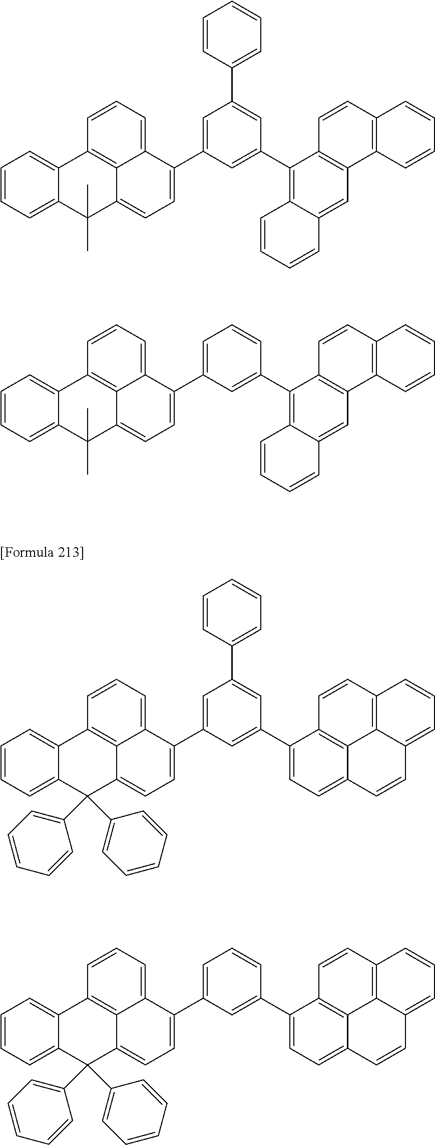

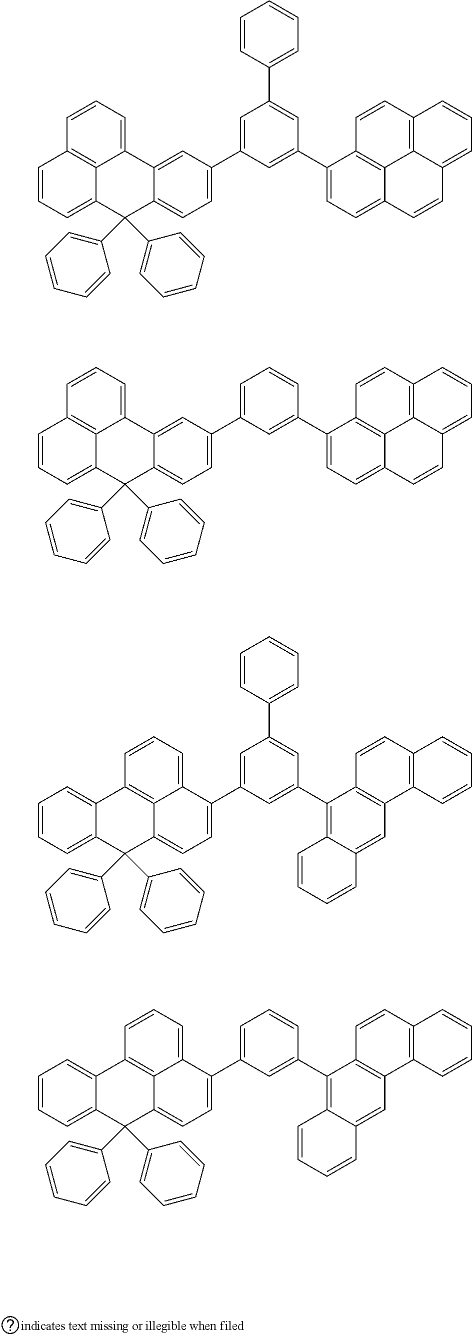

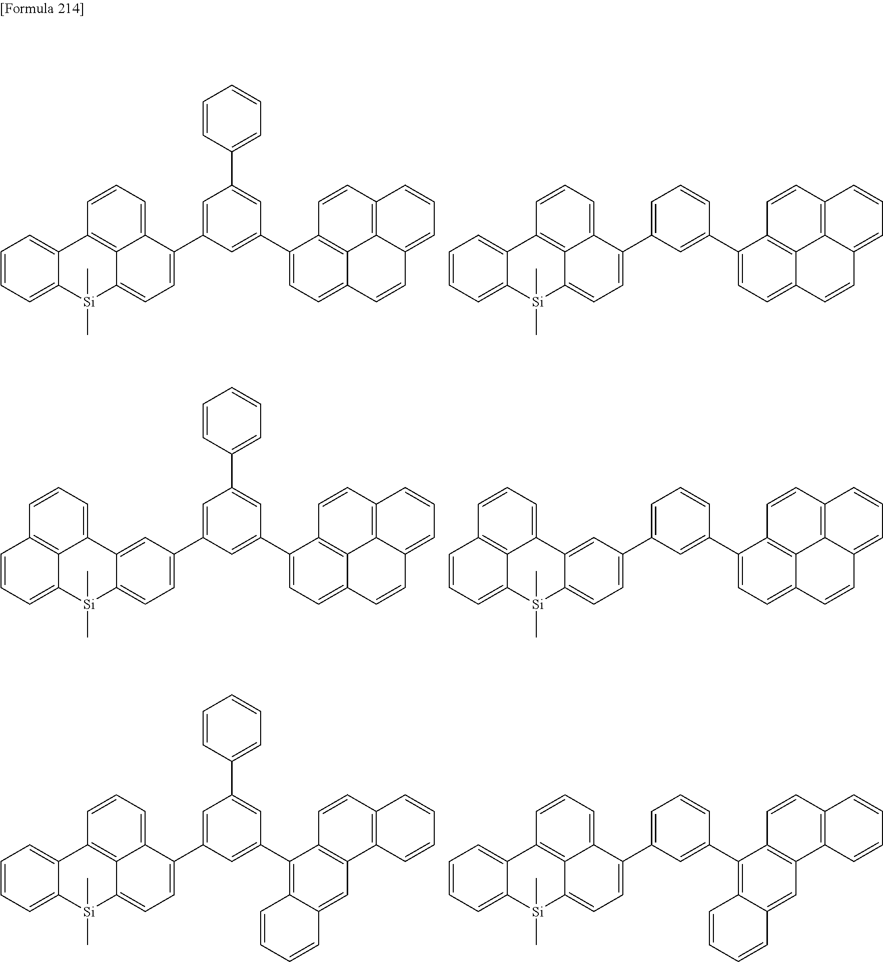

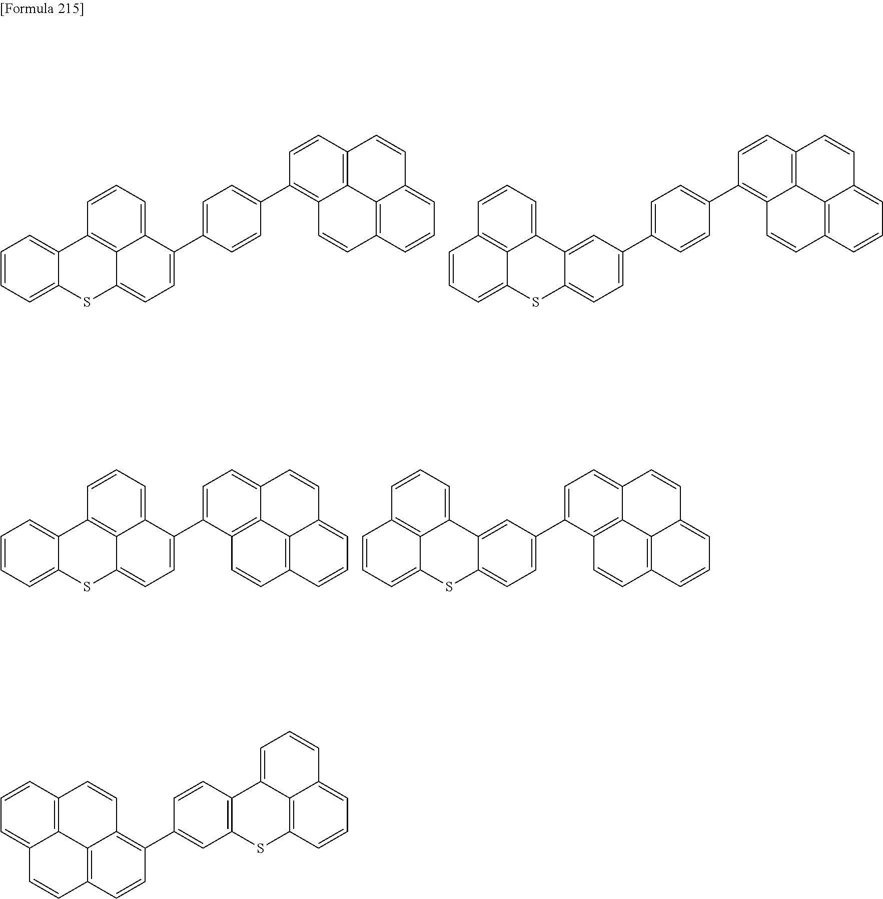

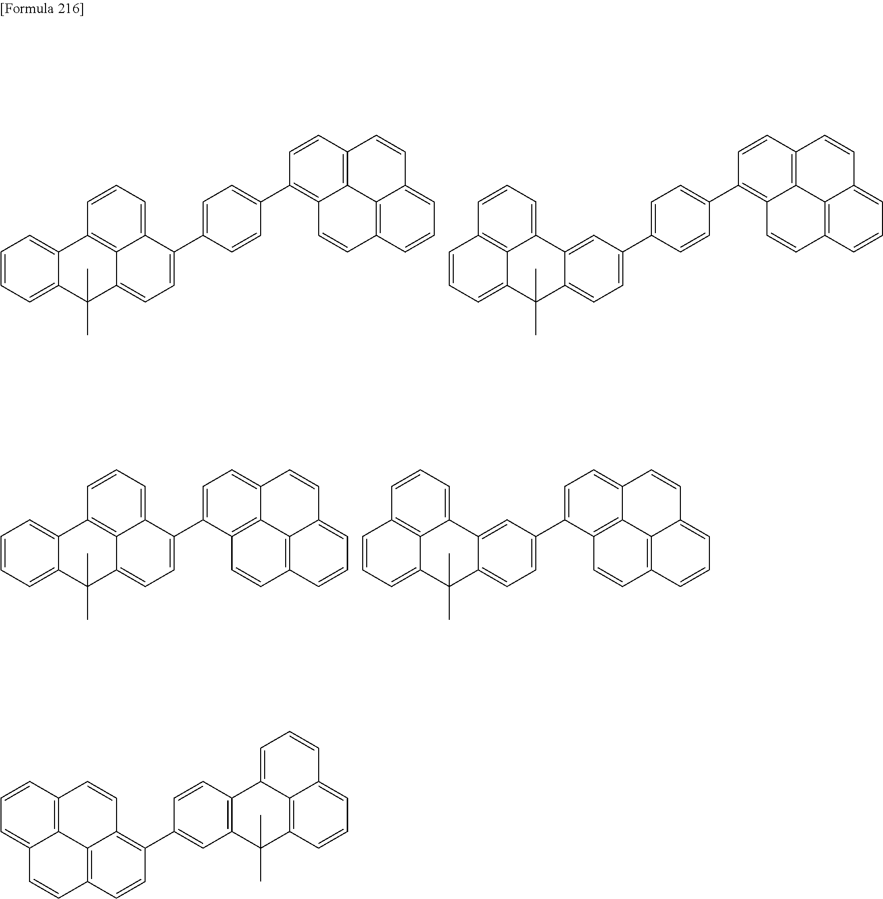

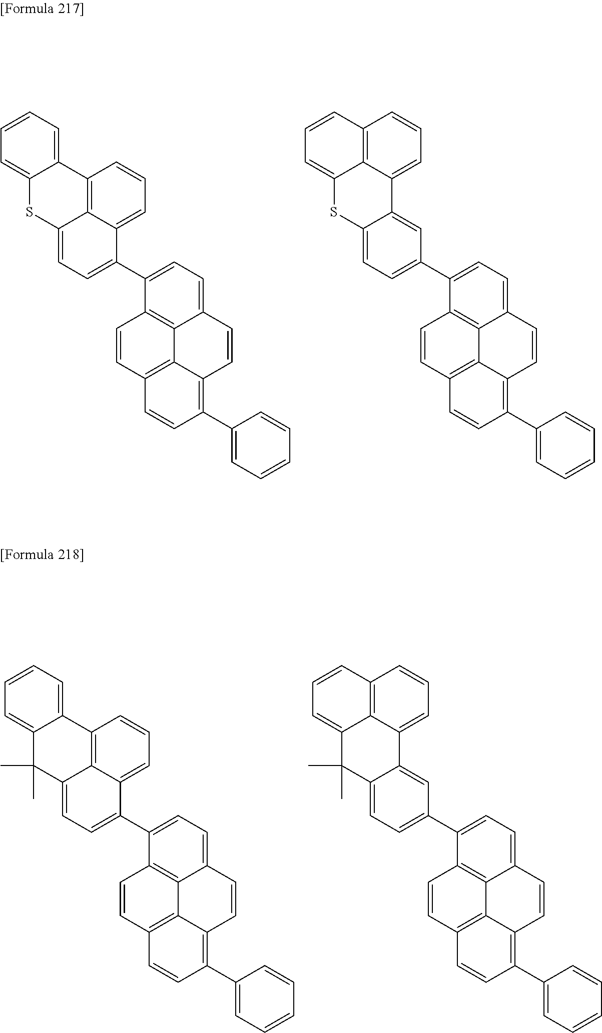

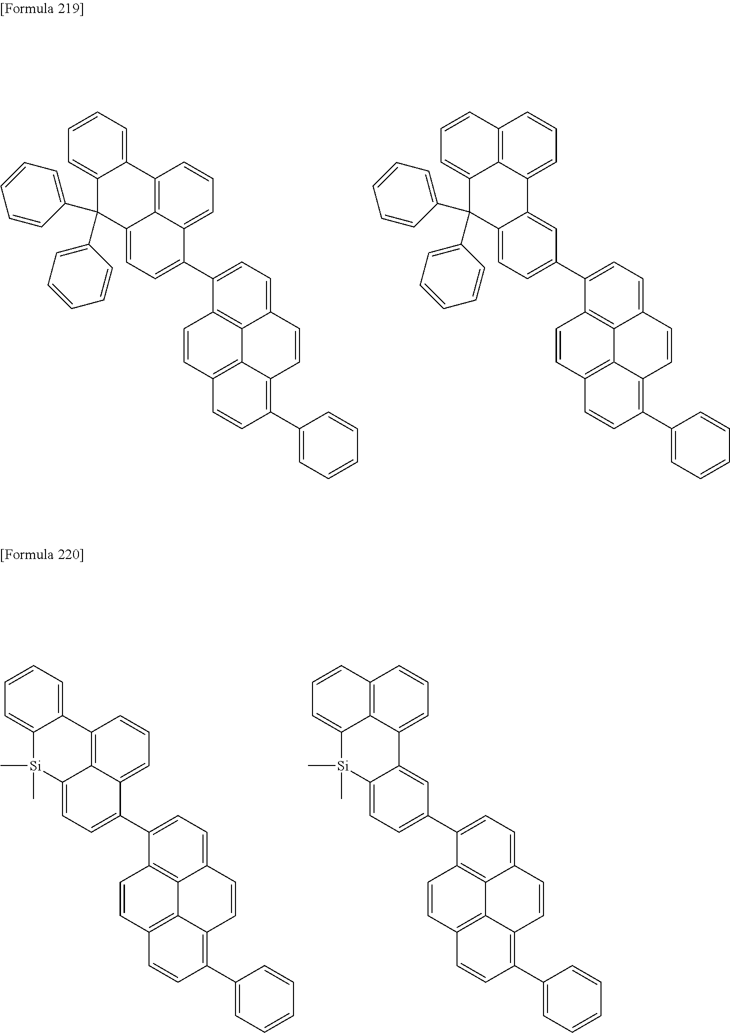

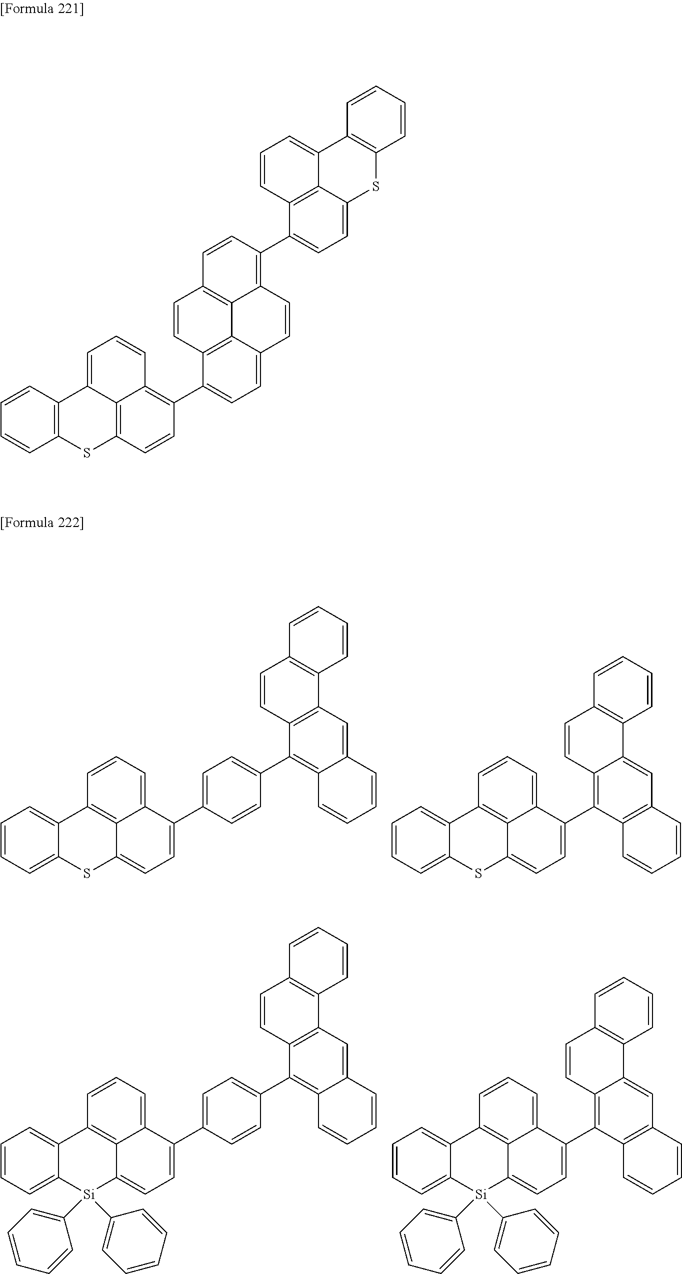

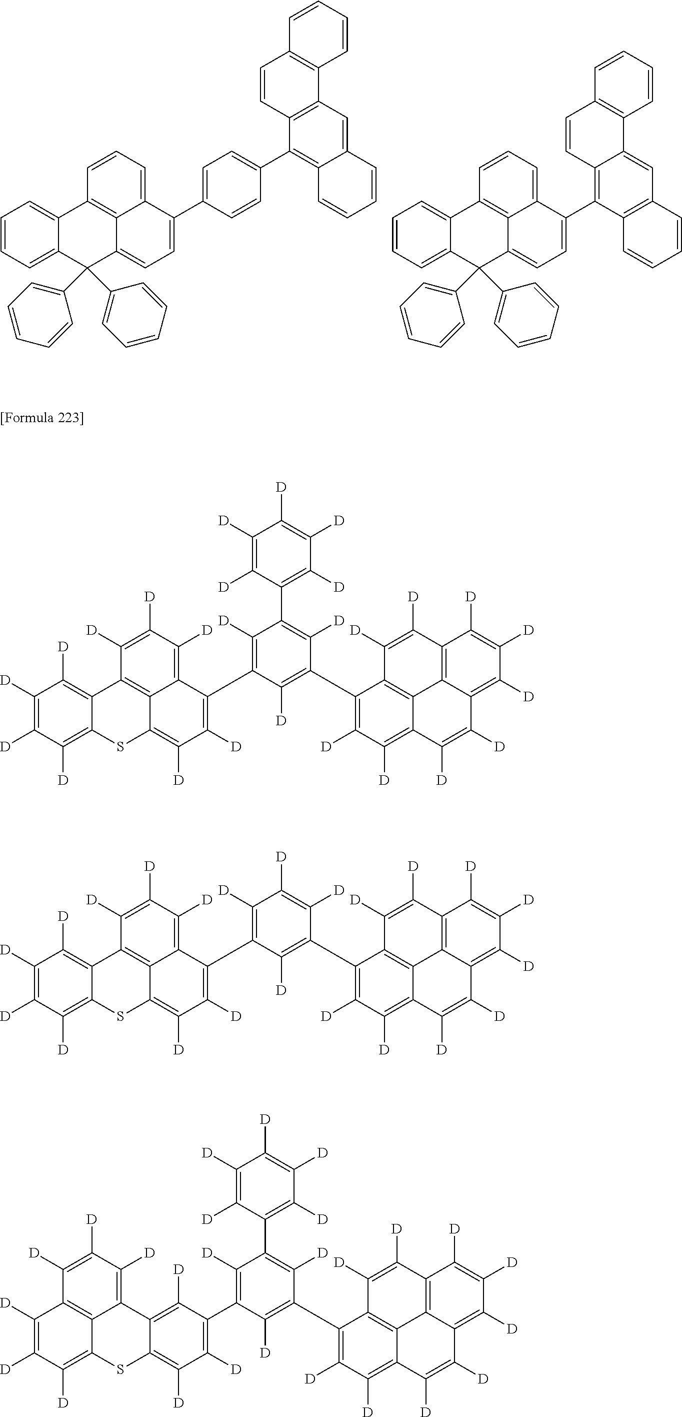

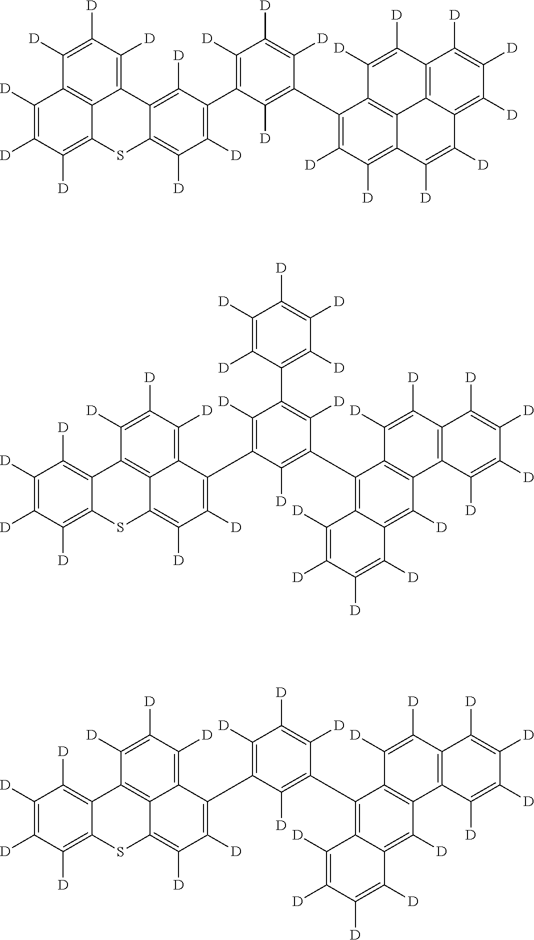

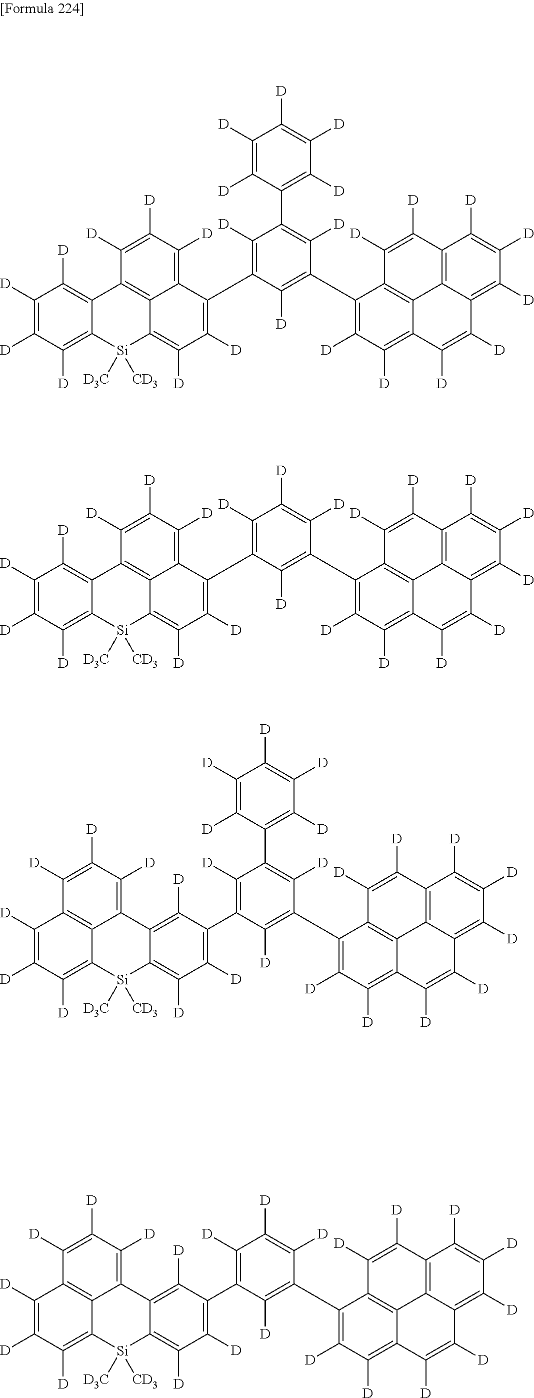

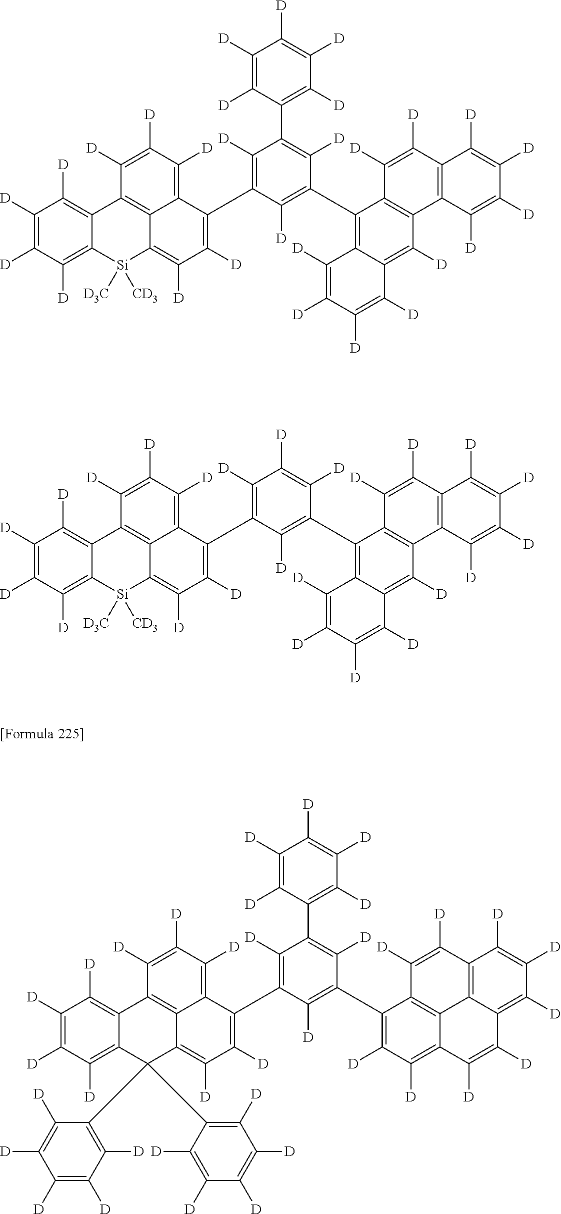

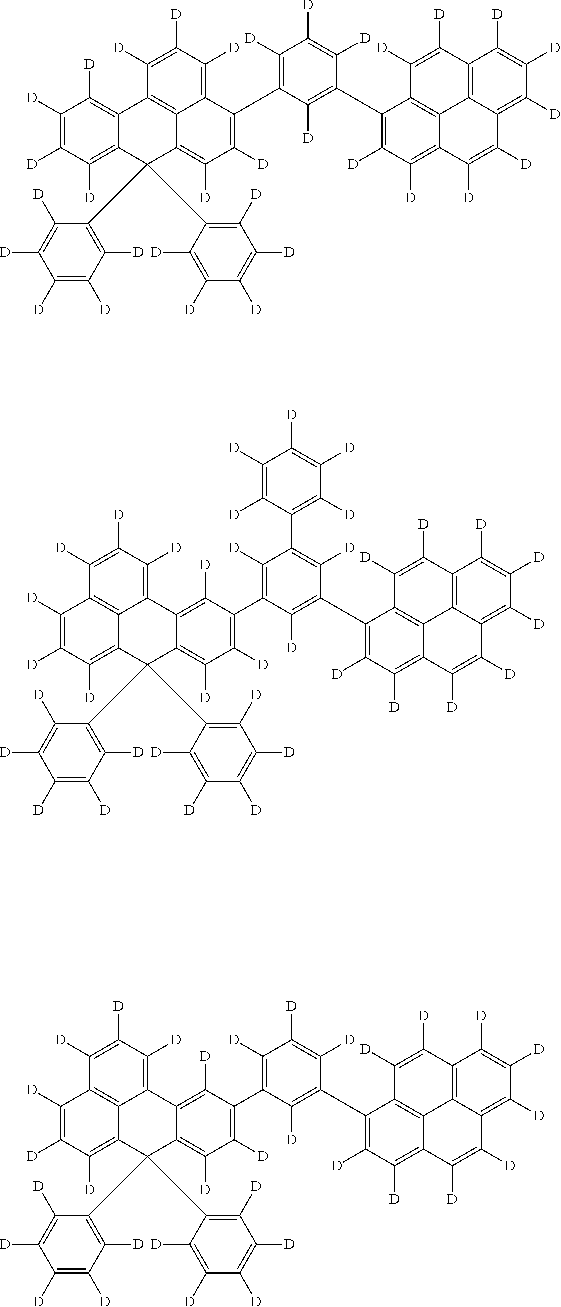

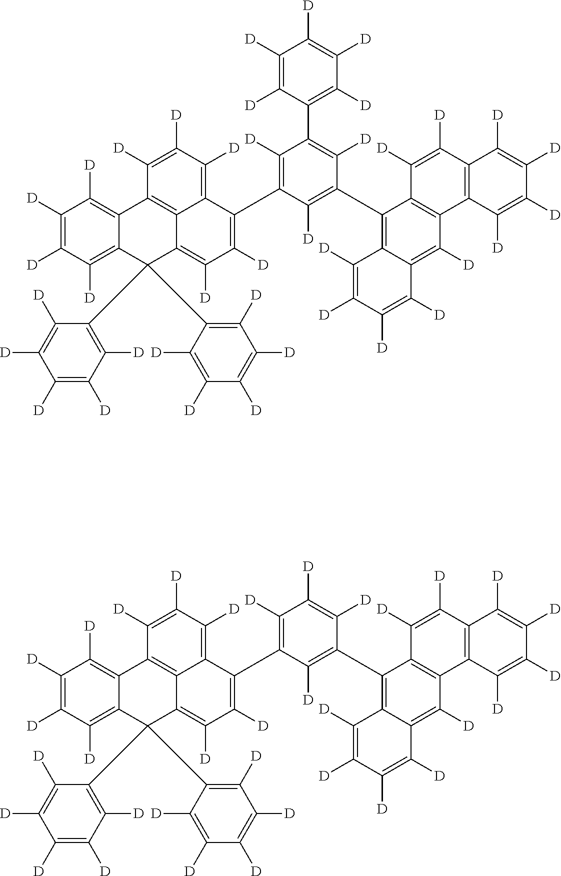

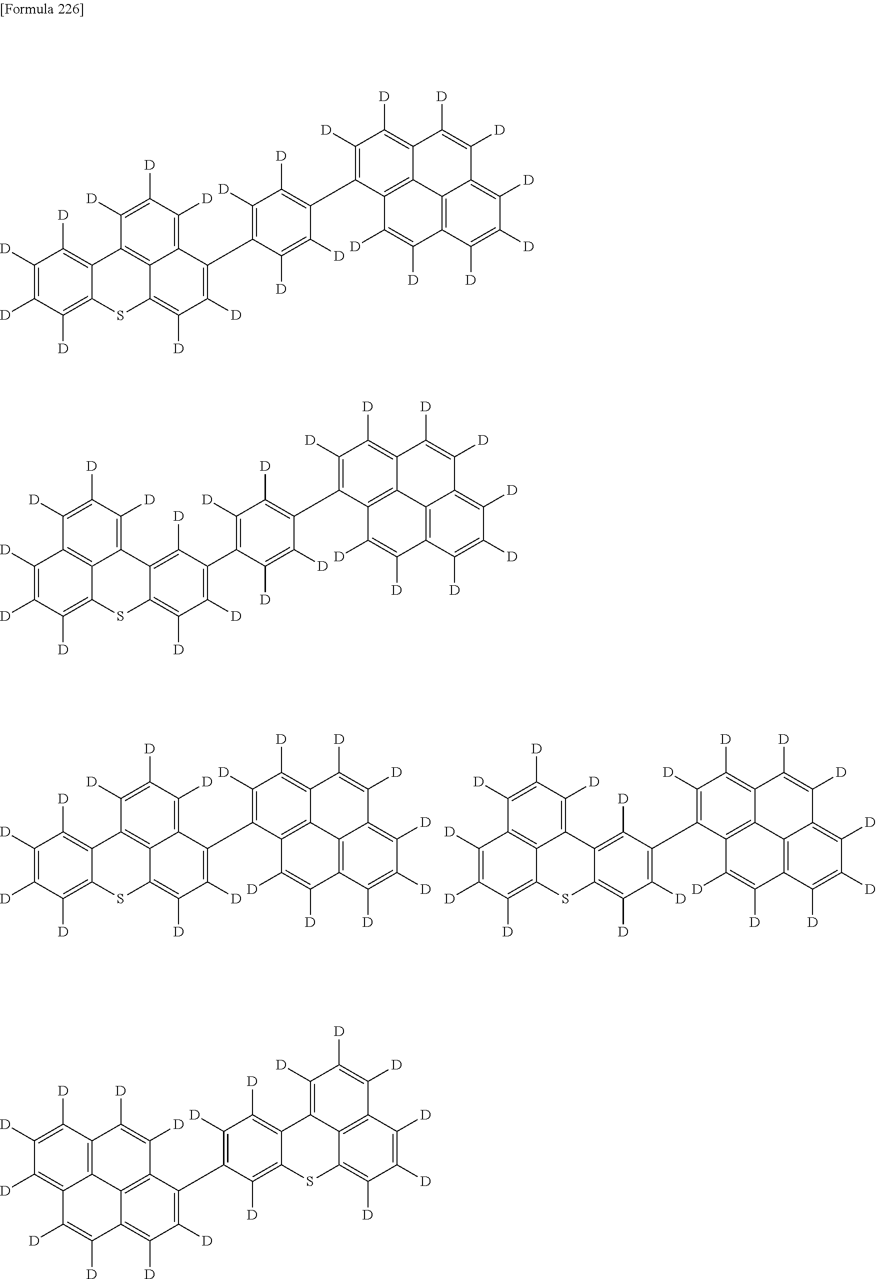

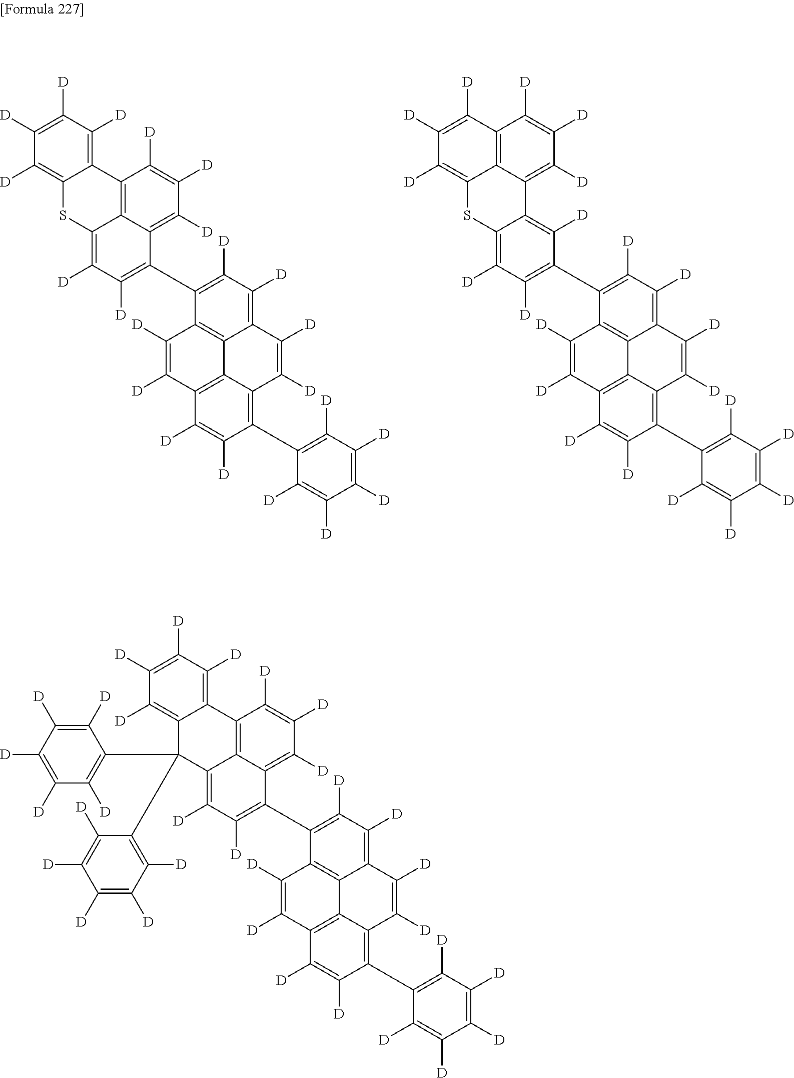

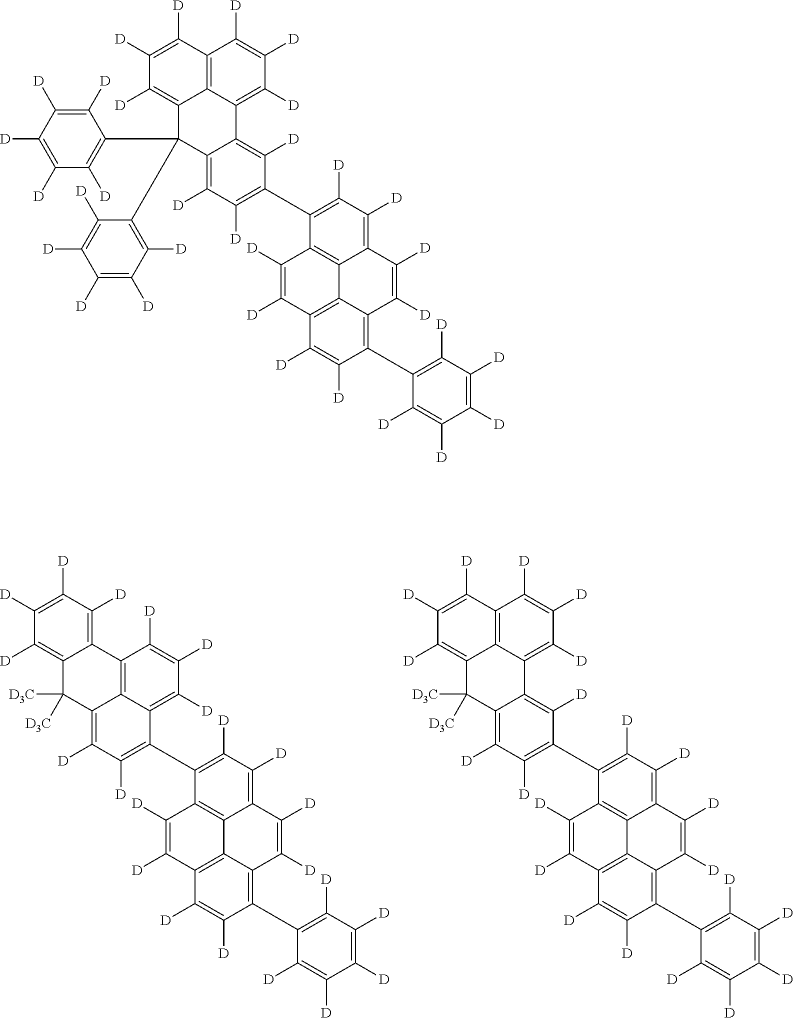

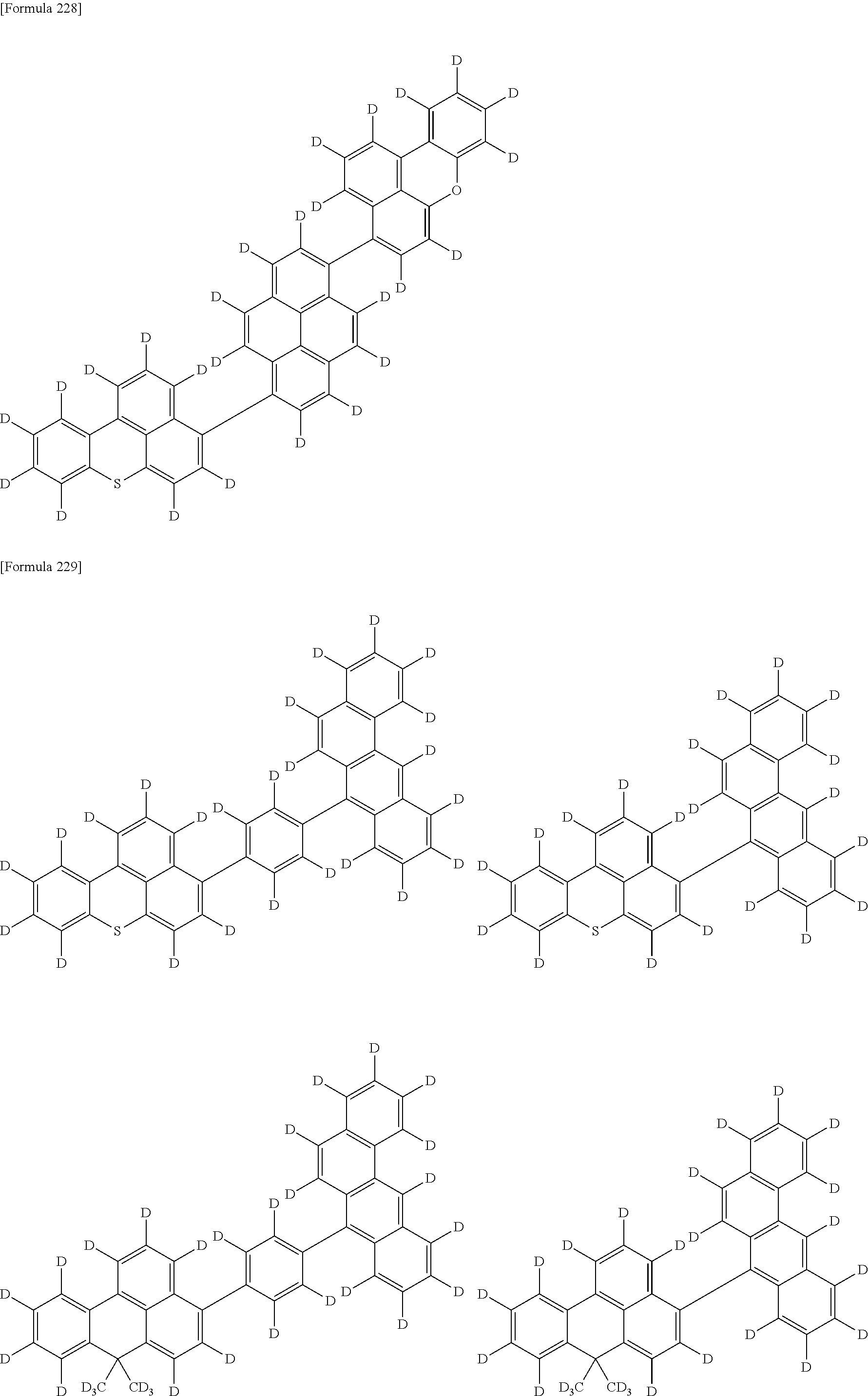

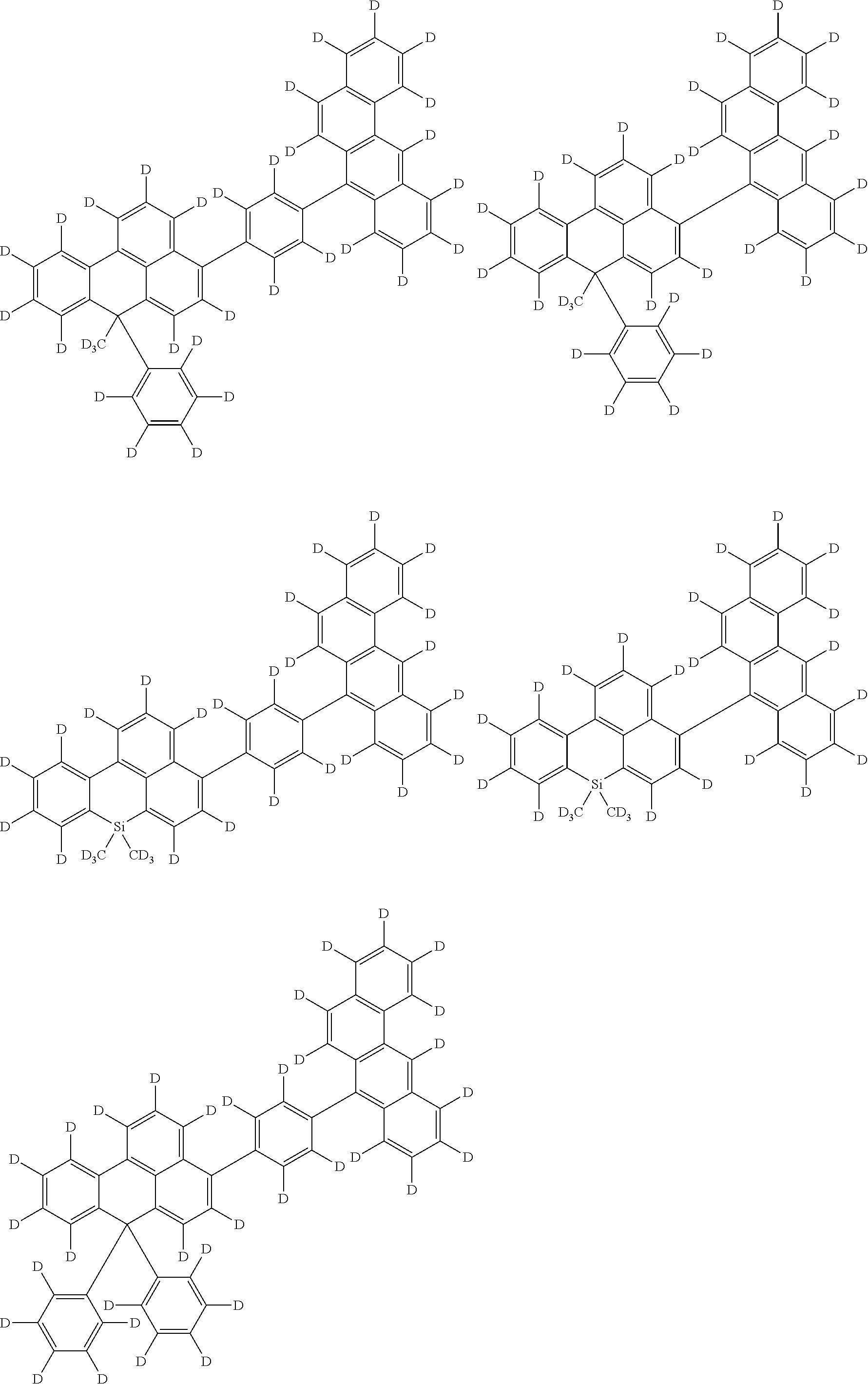

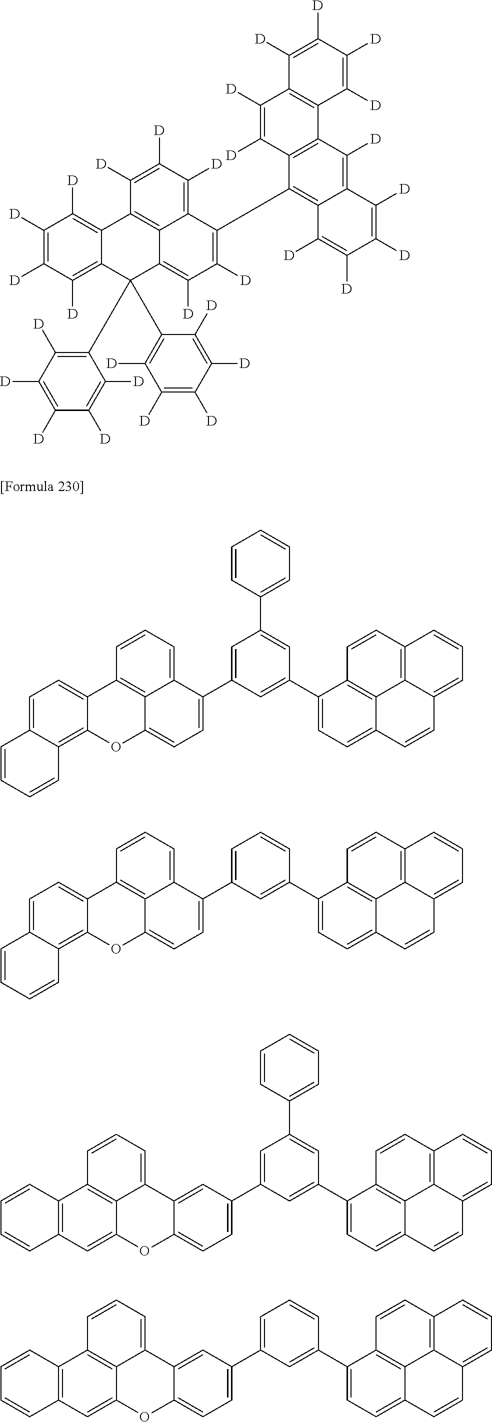

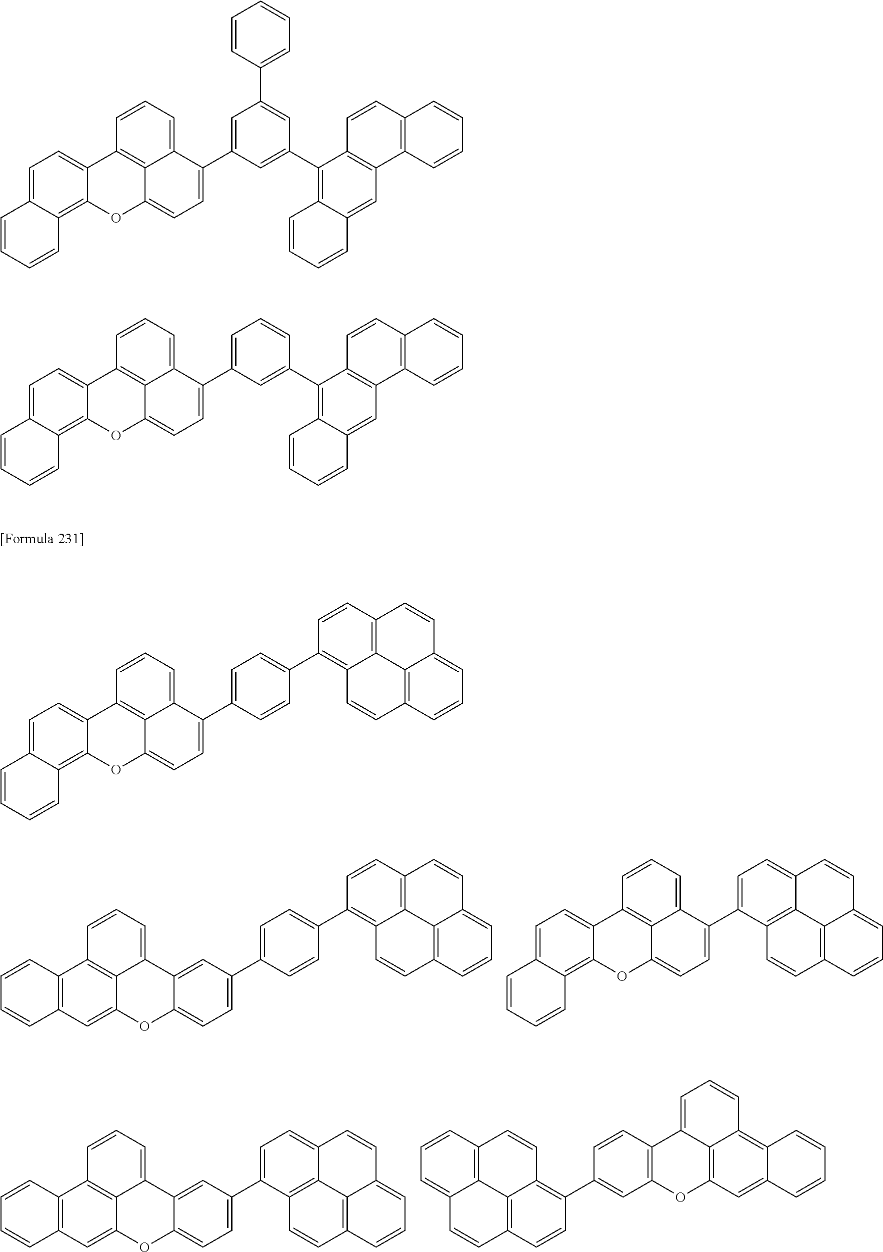

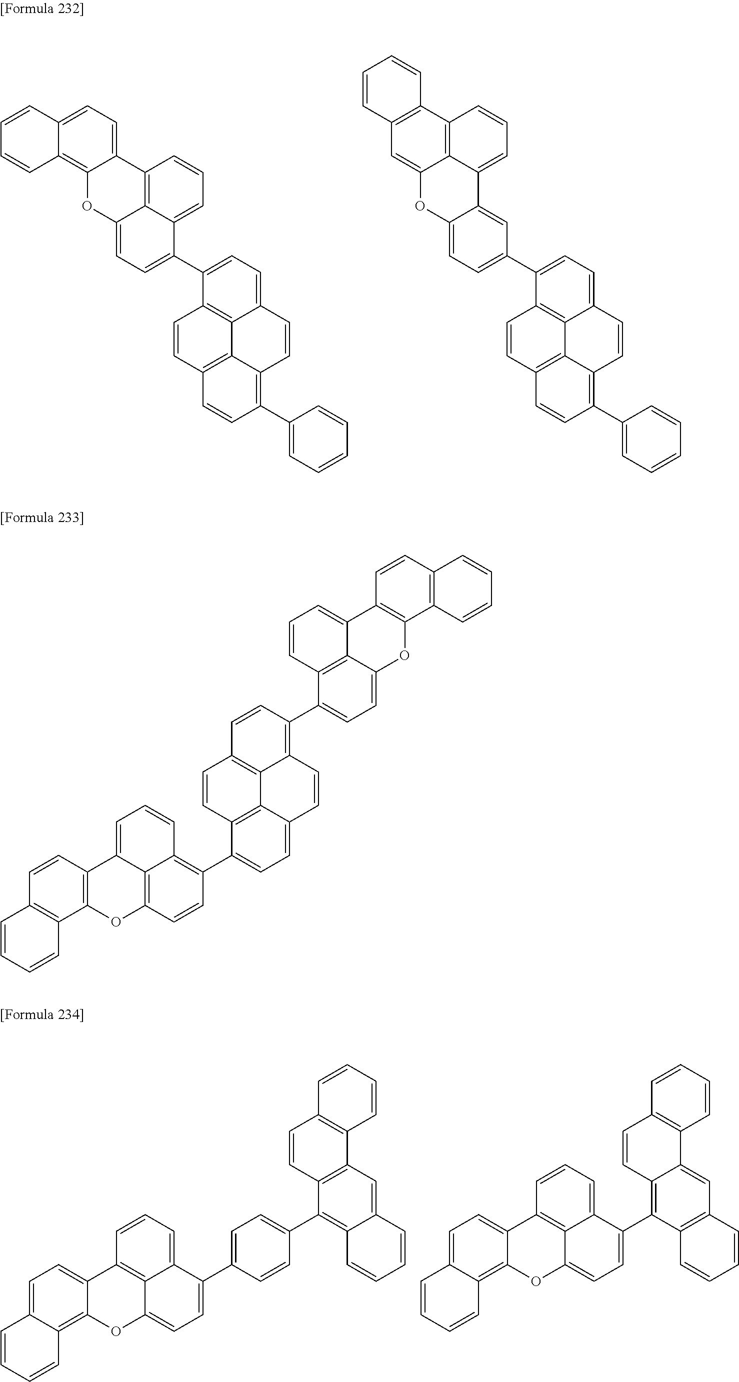

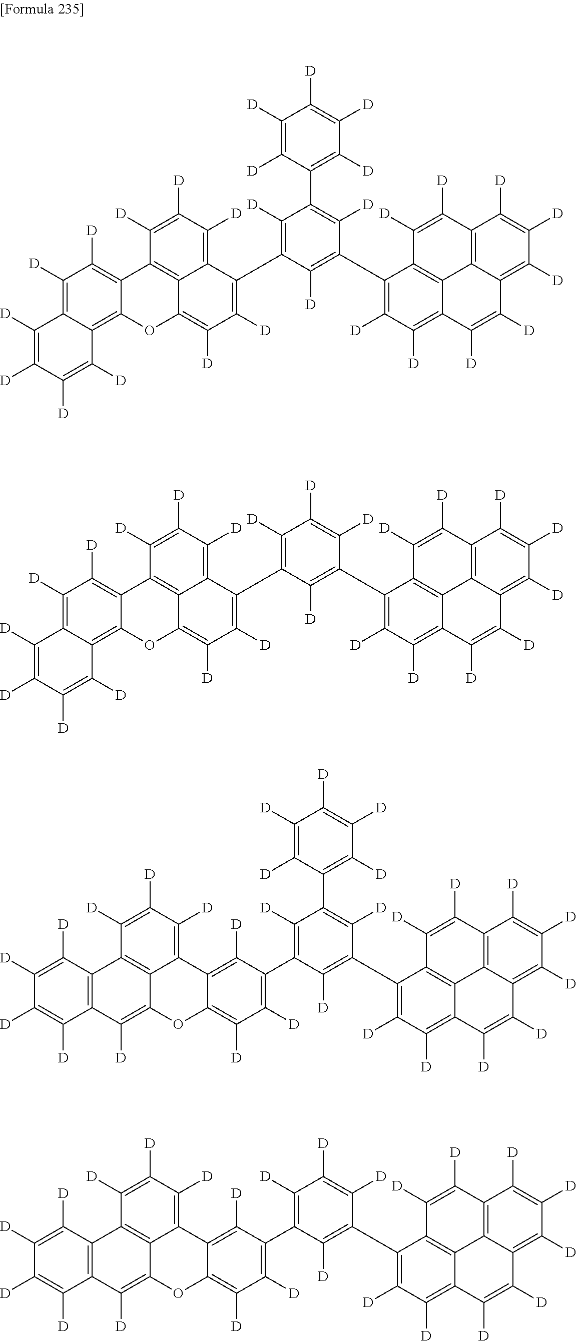

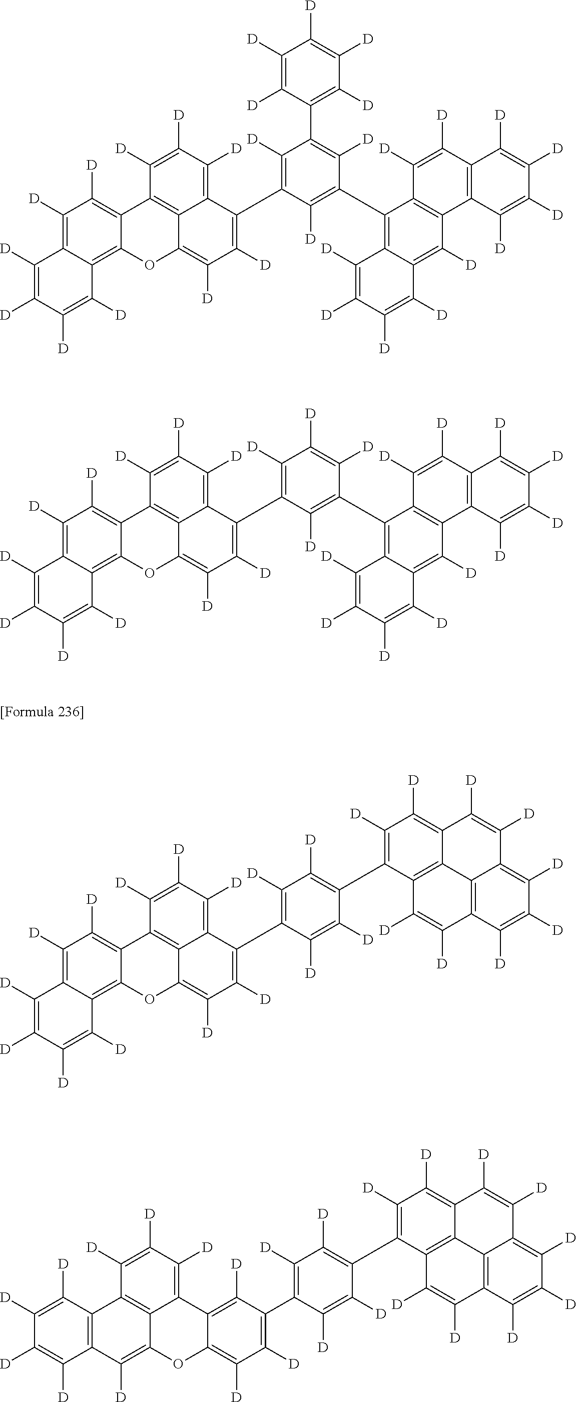

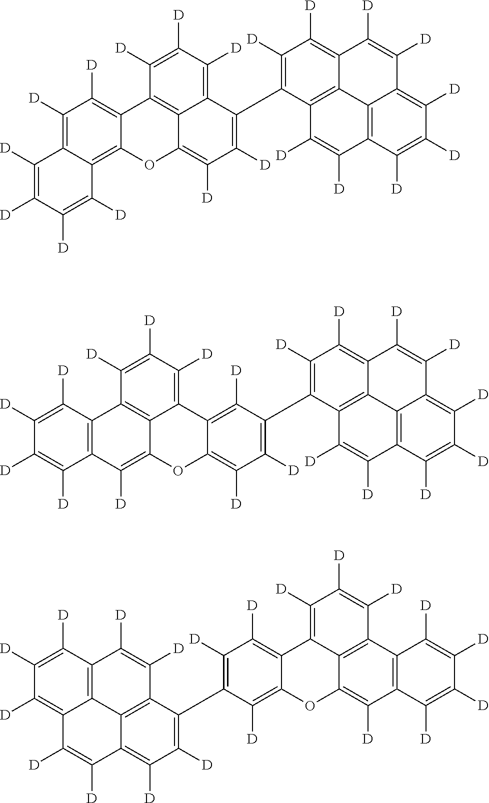

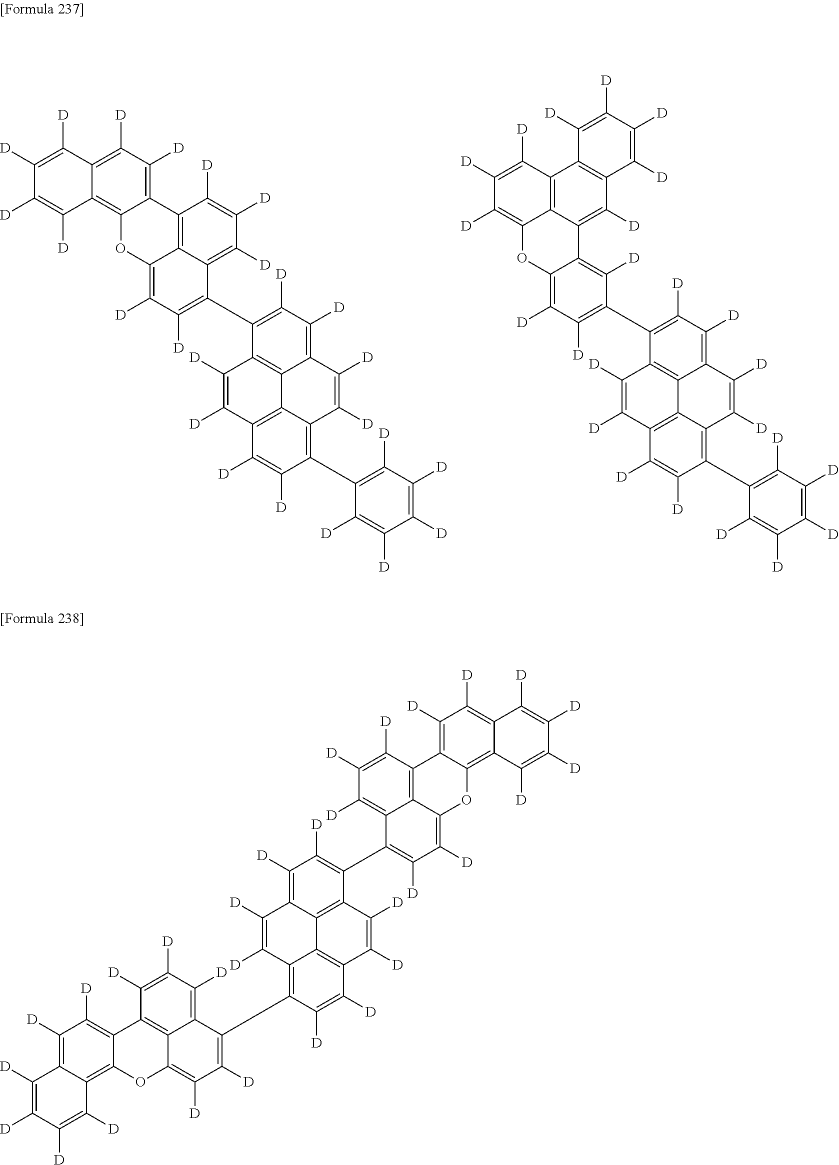

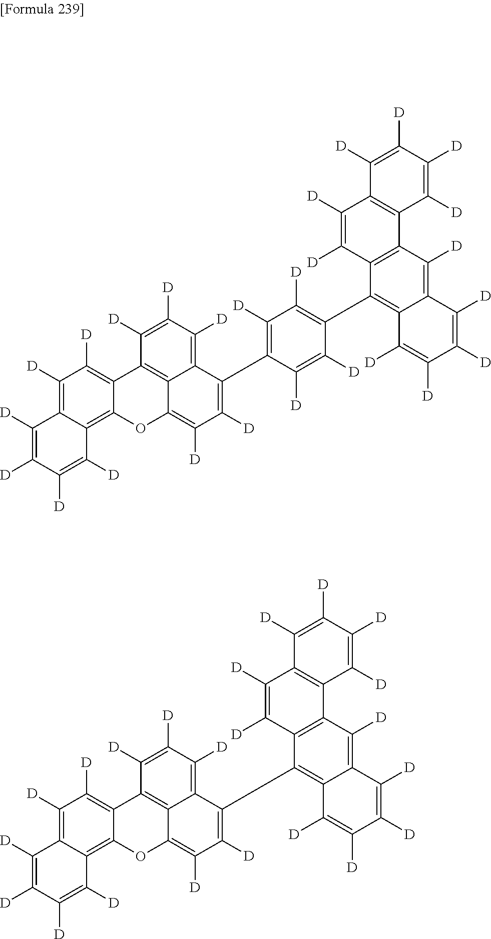 [SMILES: [2H]c1c([2H])c(-c2c3c([2H])c([2H])c([2H])c([2H])c3c([2H])c3c2c([2H])c([2H])c2c([2H])c([2H])c([2H])c([2H])c23)c([2H])c([2H])c1-c1c([2H])c([2H])c2c3c(c([2H])c([2H])c([2H])c13)-c1c([2H])c([2H])c3c([2H])c([2H])c([2H])c([2H])c3c1O2 ; [2H]c1c([2H])c([2H])c2c3c(c([2H])c([2H])c2c1[2H])-c1c([2H])c([2H])c([2H])c2c(-c4c5c([2H])c([2H])c([2H])c([2H])c5c([2H])c5c4c([2H])c([2H])c4c([2H])c([2H])c([2H])c([2H])c45)c([2H])c([2H])c(c12)O3]

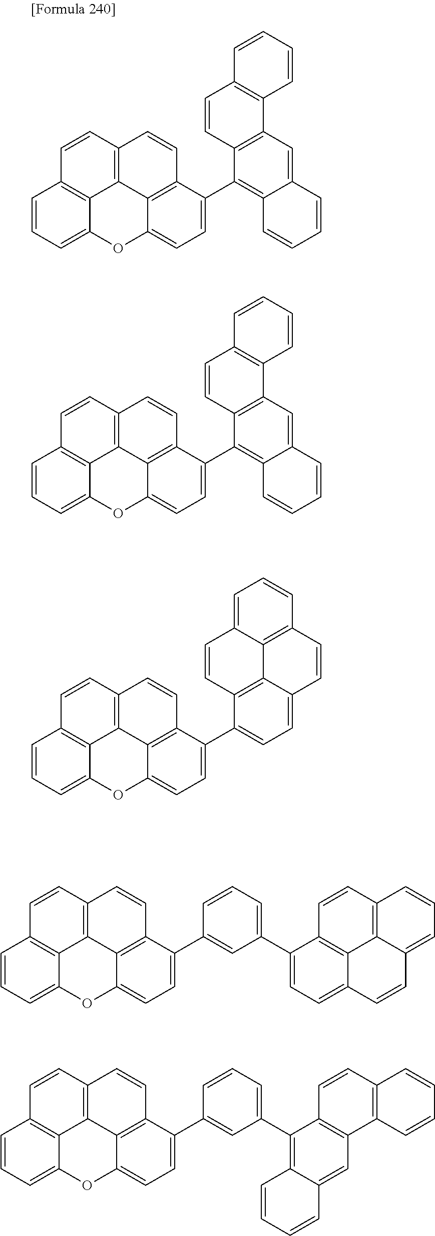 [SMILES: c1ccc2c(-c3ccc4c5c3ccc3ccc6cccc(c6c35)O4)c3ccc4ccccc4c3cc2c1 ; c1ccc2c(-c3ccc4c5c3ccc3ccc6cccc(c6c35)O4)c3ccc4ccccc4c3cc2c1 ; c1cc2c3c(c1)ccc1ccc4c(-c5ccc6ccc7cccc8ccc5c6c78)ccc(c4c13)O2 ; c1cc(-c2ccc3c4c2ccc2ccc5cccc(c5c24)O3)cc(-c2ccc3ccc4cccc5ccc2c3c45)c1 ; c1cc(-c2c3ccccc3cc3c2ccc2ccccc23)cc(-c2ccc3c4c2ccc2ccc5cccc(c5c24)O3)c1]

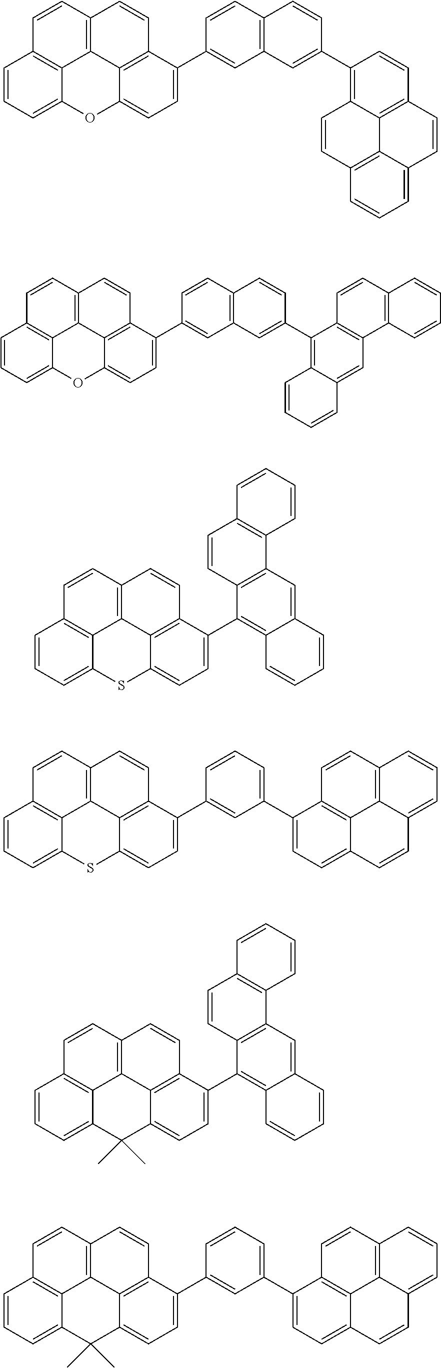

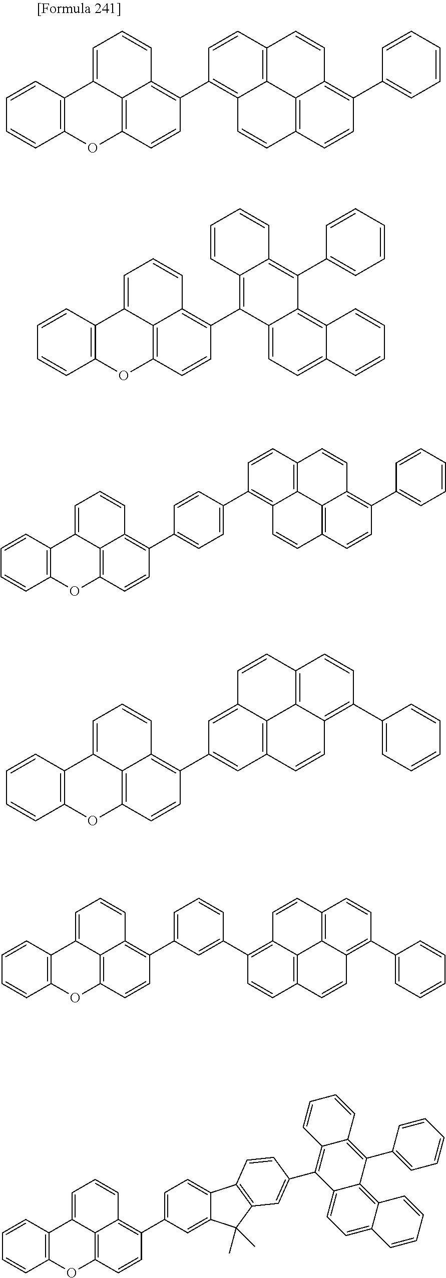 [SMILES: c1ccc(-c2ccc3ccc4c(-c5ccc6c7c(cccc57)-c5ccccc5O6)ccc5ccc2c3c54)cc1 ; c1ccc(-c2c3ccccc3c(-c3ccc4c5c(cccc35)-c3ccccc3O4)c3ccc4ccccc4c23)cc1 ; c1ccc(-c2ccc3ccc4c(-c5ccc(-c6ccc7c8c(cccc68)-c6ccccc6O7)cc5)ccc5ccc2c3c54)cc1 ; c1ccc(-c2ccc3ccc4cc(-c5ccc6c7c(cccc57)-c5ccccc5O6)cc5ccc2c3c45)cc1 ; c1ccc(-c2ccc3ccc4c(-c5cccc(-c6ccc7c8c(cccc68)-c6ccccc6O7)c5)ccc5ccc2c3c54)cc1 ; CC1(C)c2cc(-c3ccc4c5c(cccc35)-c3ccccc3O4)ccc2-c2ccc(-c3c4ccccc4c(-c4ccccc4)c4c3ccc3ccccc34)cc21]

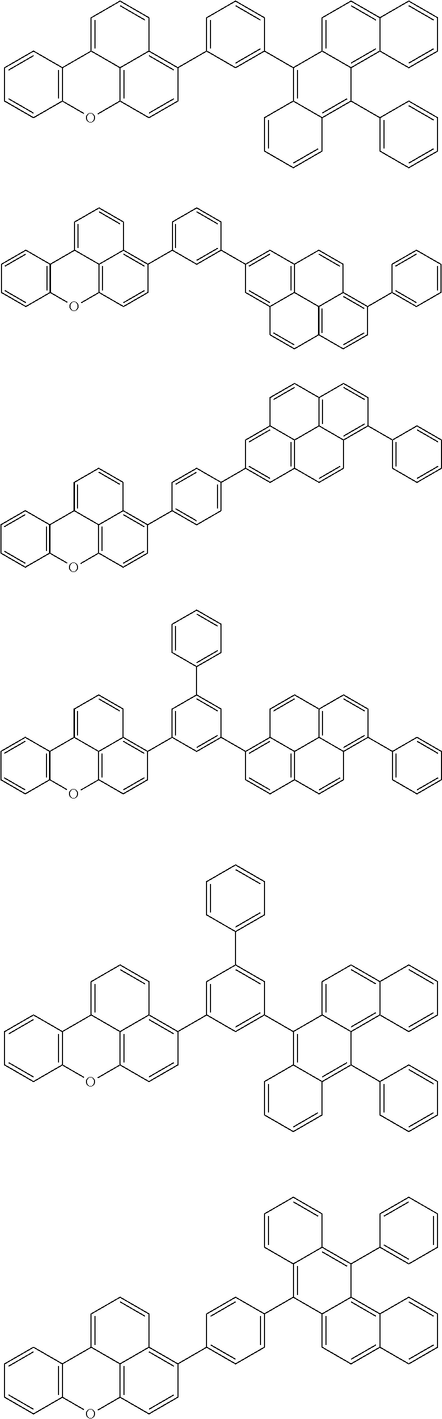

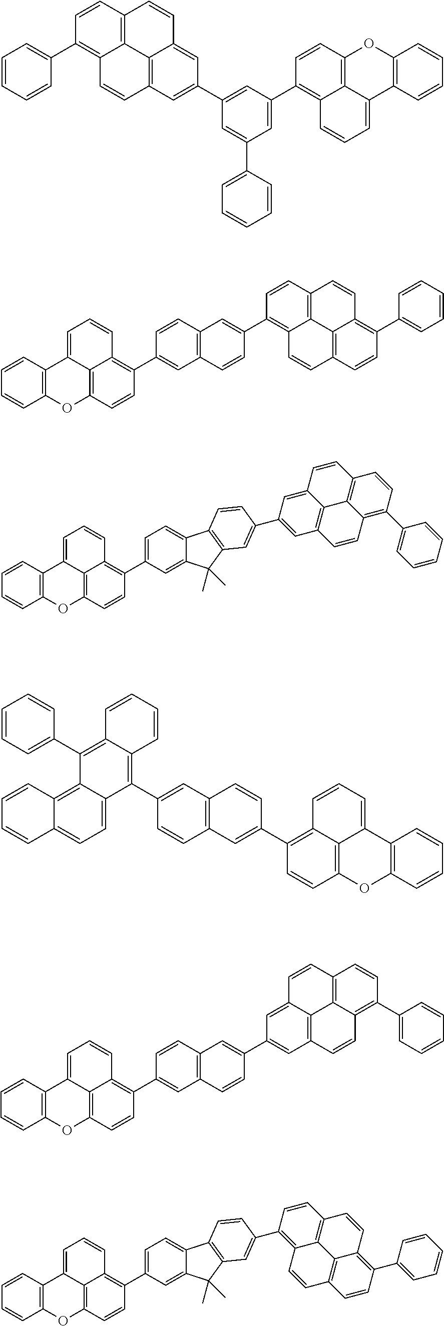

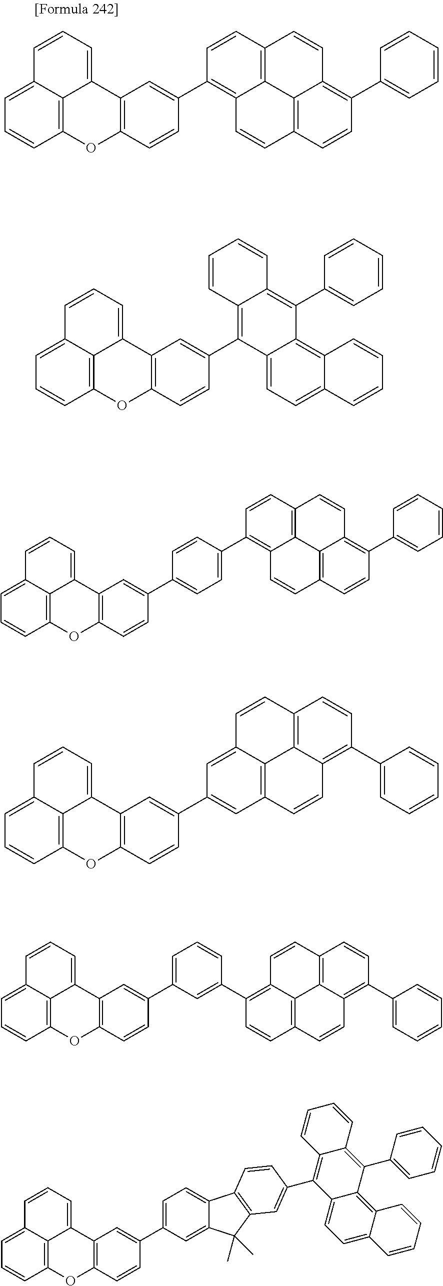 [SMILES: c1ccc(-c2ccc3ccc4c(-c5ccc6c(c5)-c5cccc7cccc(c57)O6)ccc5ccc2c3c54)cc1 ; c1ccc(-c2c3ccccc3c(-c3ccc4c(c3)-c3cccc5cccc(c35)O4)c3ccc4ccccc4c23)cc1 ; c1ccc(-c2ccc3ccc4c(-c5ccc(-c6ccc7c(c6)-c6cccc8cccc(c68)O7)cc5)ccc5ccc2c3c54)cc1 ; c1ccc(-c2ccc3ccc4cc(-c5ccc6c(c5)-c5cccc7cccc(c57)O6)cc5ccc2c3c45)cc1 ; c1ccc(-c2ccc3ccc4c(-c5cccc(-c6ccc7c(c6)-c6cccc8cccc(c68)O7)c5)ccc5ccc2c3c54)cc1 ; CC1(C)c2cc(-c3ccc4c(c3)-c3cccc5cccc(c35)O4)ccc2-c2ccc(-c3c4ccccc4c(-c4ccccc4)c4c3ccc3ccccc34)cc21]

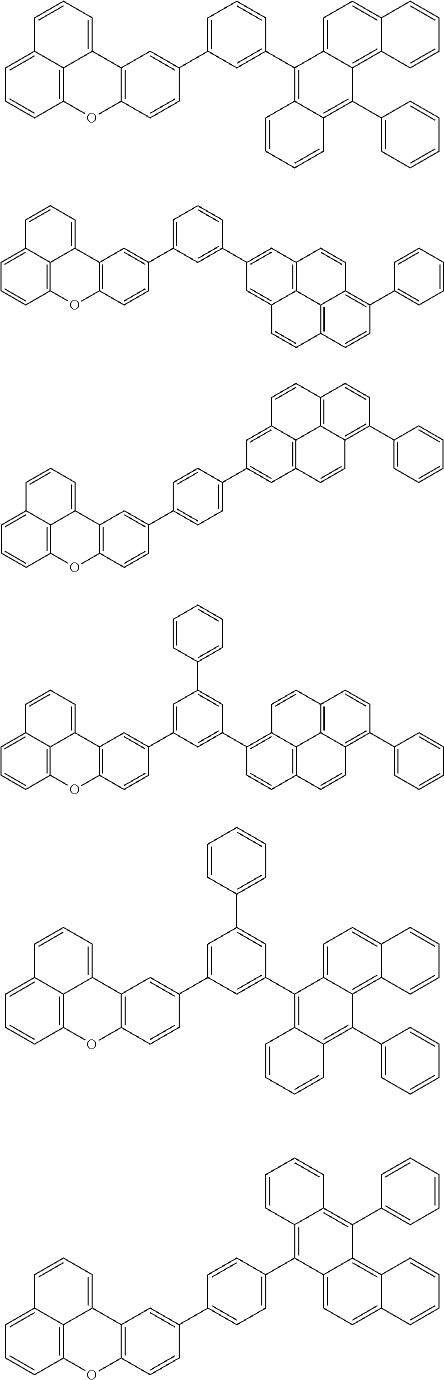

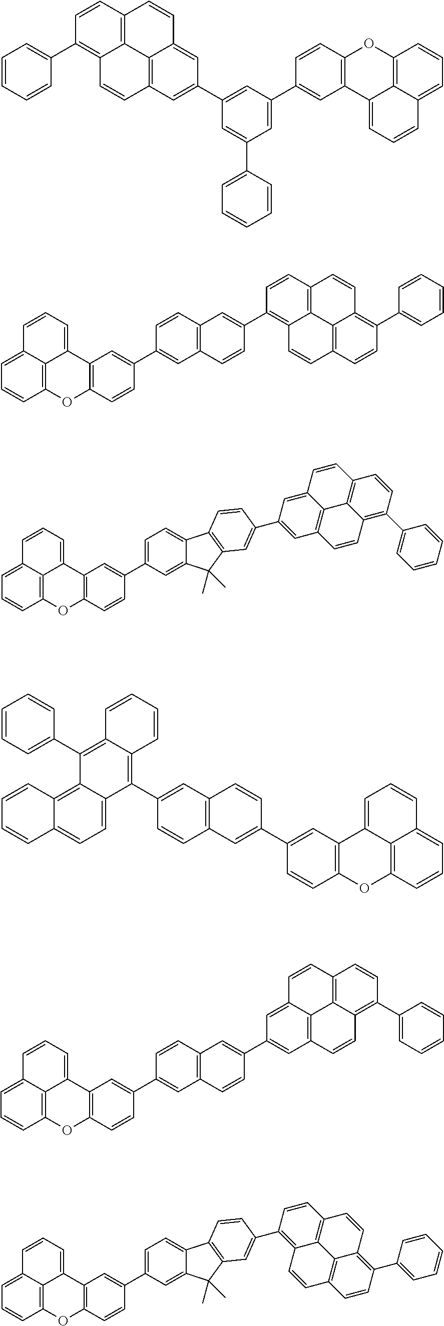

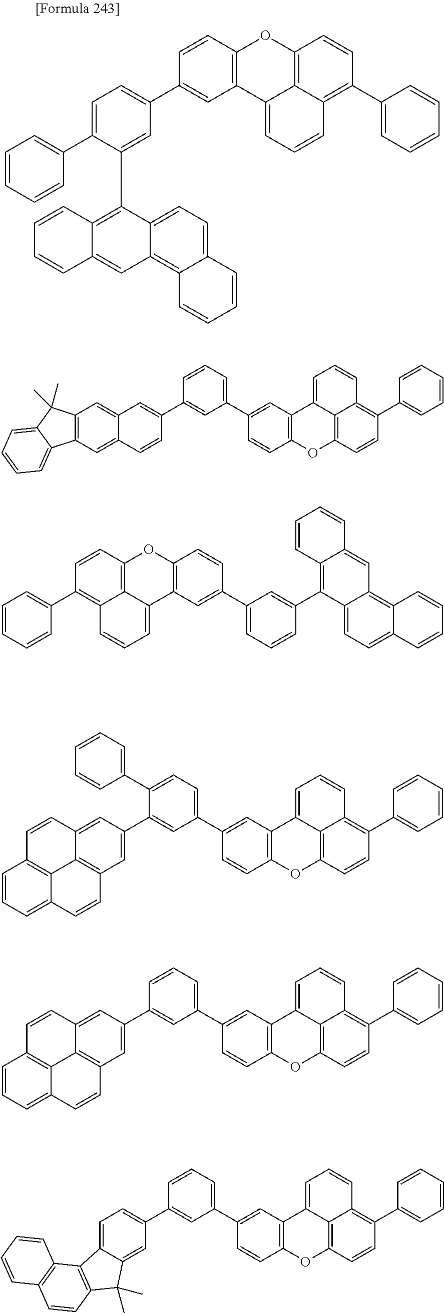

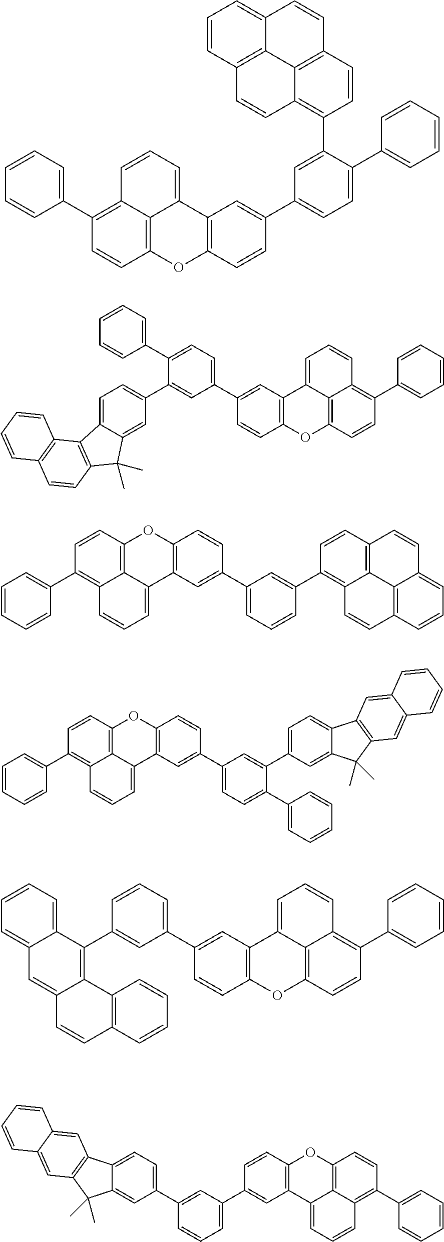

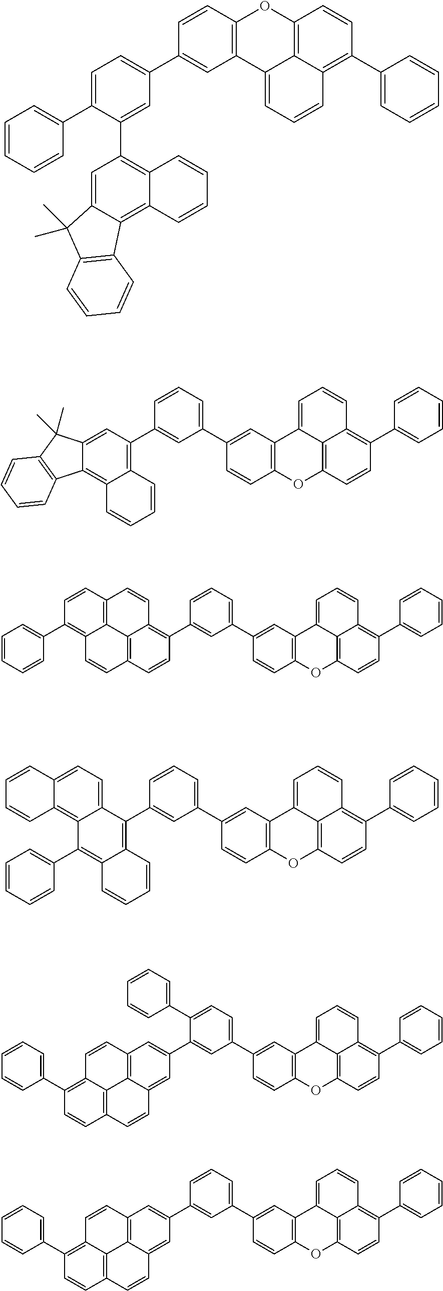

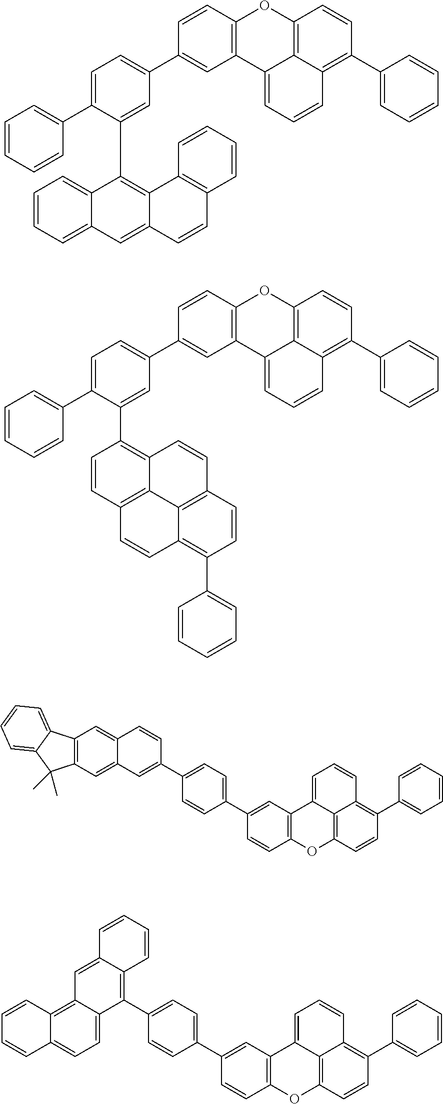 [SMILES: c1ccc(-c2ccc(-c3ccc4c(c3)-c3cccc5c(-c6ccccc6)ccc(c35)O4)cc2-c2c3ccccc3cc3ccc4ccccc4c23)cc1 ; c1ccc(-c2ccc(-c3ccc4c(c3)-c3cccc5c(-c6ccccc6)ccc(c35)O4)cc2-c2ccc3ccc4c(-c5ccccc5)ccc5ccc2c3c54)cc1 ; CC1(C)c2ccccc2-c2cc3ccc(-c4ccc(-c5ccc6c(c5)-c5cccc7c(-c8ccccc8)ccc(c57)O6)cc4)cc3cc21 ; c1ccc(-c2ccc3c4c(cccc24)-c2cc(-c4ccc(-c5c6ccccc6cc6c5ccc5ccccc56)cc4)ccc2O3)cc1]

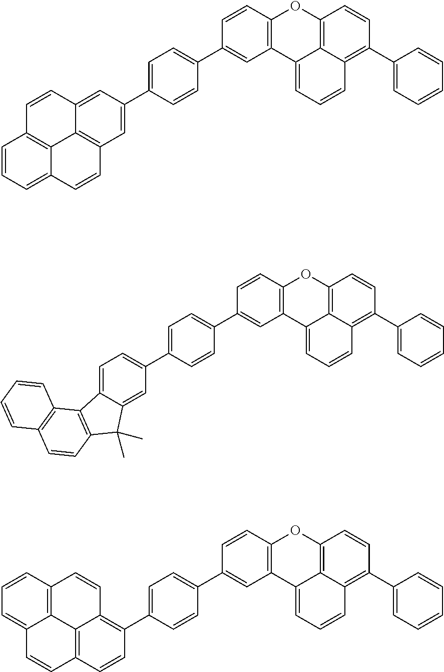 [SMILES: c1ccc(-c2ccc3c4c(cccc24)-c2cc(-c4ccc(-c5cc6ccc7cccc8ccc(c5)c6c78)cc4)ccc2O3)cc1 ; CC1(C)c2cc(-c3ccc(-c4ccc5c(c4)-c4cccc6c(-c7ccccc7)ccc(c46)O5)cc3)ccc2-c2c1ccc1ccccc21 ; c1ccc(-c2ccc3c4c(cccc24)-c2cc(-c4ccc(-c5ccc6ccc7cccc8ccc5c6c78)cc4)ccc2O3)cc1]

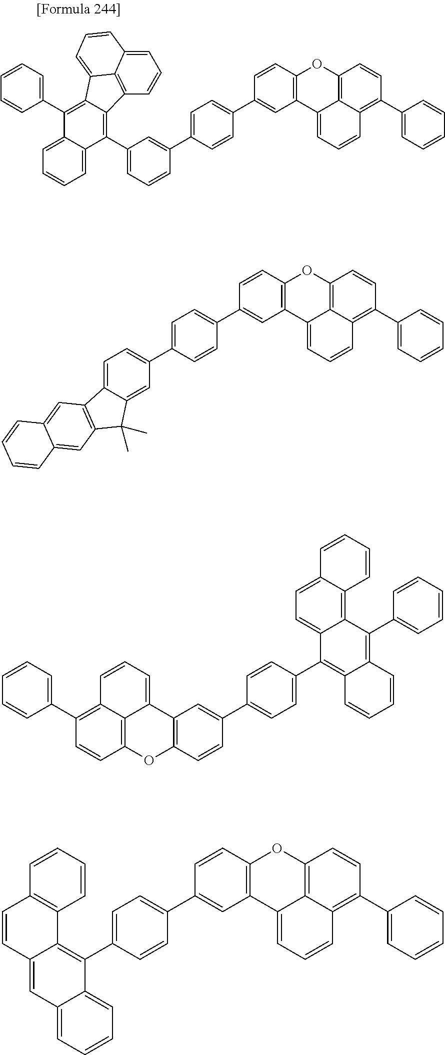 [SMILES: c1ccc(-c2c3c(c(-c4cccc(-c5ccc(-c6ccc7c(c6)-c6cccc8c(-c9ccccc9)ccc(c68)O7)cc5)c4)c4ccccc24)-c2cccc4cccc-3c24)cc1 ; CC1(C)c2cc(-c3ccc(-c4ccc5c(c4)-c4cccc6c(-c7ccccc7)ccc(c46)O5)cc3)ccc2-c2cc3ccccc3cc21 ; c1ccc(-c2ccc3c4c(cccc24)-c2cc(-c4ccc(-c5c6ccccc6c(-c6ccccc6)c6c5ccc5ccccc56)cc4)ccc2O3)cc1 ; c1ccc(-c2ccc3c4c(cccc24)-c2cc(-c4ccc(-c5c6ccccc6cc6ccc7ccccc7c56)cc4)ccc2O3)cc1]

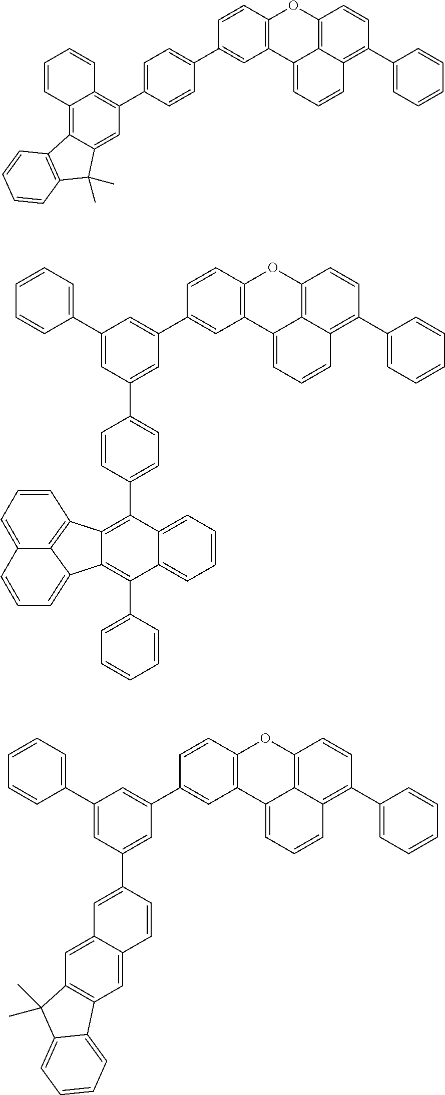 [SMILES: CC1(C)c2ccccc2-c2c1cc(-c1ccc(-c3ccc4c(c3)-c3cccc5c(-c6ccccc6)ccc(c35)O4)cc1)c1ccccc21 ; c1ccc(-c2cc(-c3ccc(-c4c5c(c(-c6ccccc6)c6ccccc46)-c4cccc6cccc-5c46)cc3)cc(-c3ccc4c(c3)-c3cccc5c(-c6ccccc6)ccc(c35)O4)c2)cc1 ; CC1(C)c2ccccc2-c2cc3ccc(-c4cc(-c5ccccc5)cc(-c5ccc6c(c5)-c5cccc7c(-c8ccccc8)ccc(c57)O6)c4)cc3cc21]

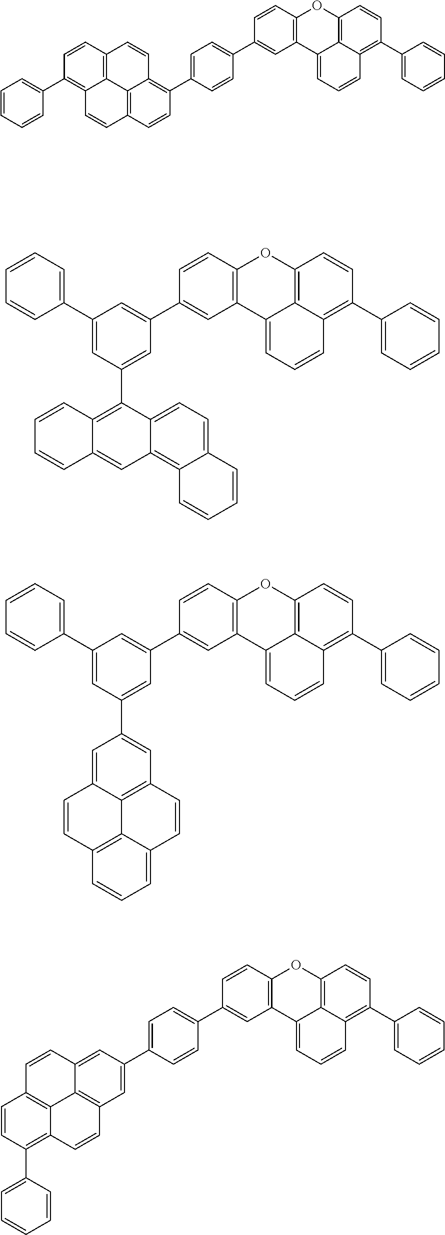 [SMILES: c1ccc(-c2ccc3c4c(cccc24)-c2cc(-c4ccc(-c5ccc6ccc7c(-c8ccccc8)ccc8ccc5c6c87)cc4)ccc2O3)cc1 ; c1ccc(-c2cc(-c3ccc4c(c3)-c3cccc5c(-c6ccccc6)ccc(c35)O4)cc(-c3c4ccccc4cc4c3ccc3ccccc34)c2)cc1 ; c1ccc(-c2cc(-c3ccc4c(c3)-c3cccc5c(-c6ccccc6)ccc(c35)O4)cc(-c3cc4ccc5cccc6ccc(c3)c4c56)c2)cc1 ; c1ccc(-c2ccc3c4c(cccc24)-c2cc(-c4ccc(-c5cc6ccc7ccc(-c8ccccc8)c8ccc(c5)c6c78)cc4)ccc2O3)cc1]

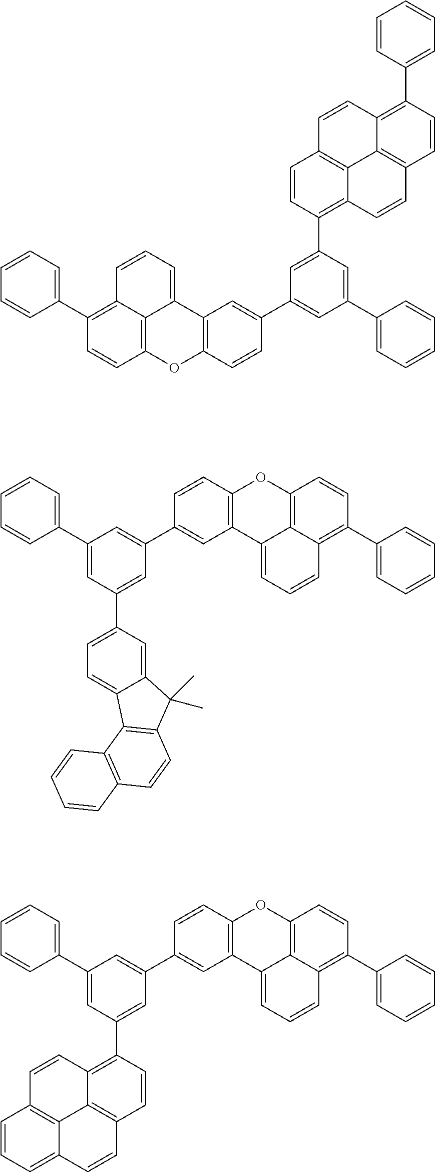 [SMILES: c1ccc(-c2cc(-c3ccc4c(c3)-c3cccc5c(-c6ccccc6)ccc(c35)O4)cc(-c3ccc4ccc5c(-c6ccccc6)ccc6ccc3c4c65)c2)cc1 ; CC1(C)c2cc(-c3cc(-c4ccccc4)cc(-c4ccc5c(c4)-c4cccc6c(-c7ccccc7)ccc(c46)O5)c3)ccc2-c2c1ccc1ccccc21 ; c1ccc(-c2cc(-c3ccc4c(c3)-c3cccc5c(-c6ccccc6)ccc(c35)O4)cc(-c3ccc4ccc5cccc6ccc3c4c56)c2)cc1]

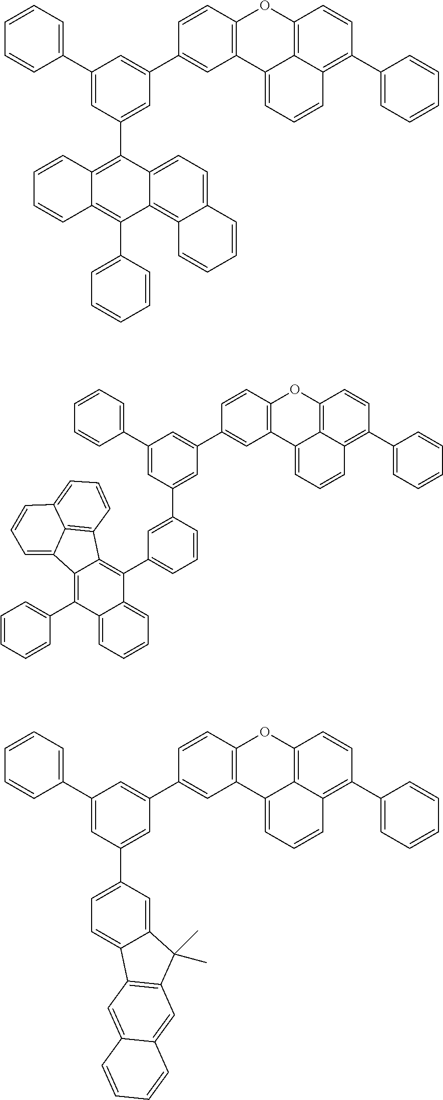 [SMILES: c1ccc(-c2cc(-c3ccc4c(c3)-c3cccc5c(-c6ccccc6)ccc(c35)O4)cc(-c3c4ccccc4c(-c4ccccc4)c4c3ccc3ccccc34)c2)cc1 ; c1ccc(-c2cc(-c3cccc(-c4c5c(c(-c6ccccc6)c6ccccc46)-c4cccc6cccc-5c46)c3)cc(-c3ccc4c(c3)-c3cccc5c(-c6ccccc6)ccc(c35)O4)c2)cc1 ; CC1(C)c2cc(-c3cc(-c4ccccc4)cc(-c4ccc5c(c4)-c4cccc6c(-c7ccccc7)ccc(c46)O5)c3)ccc2-c2cc3ccccc3cc21]

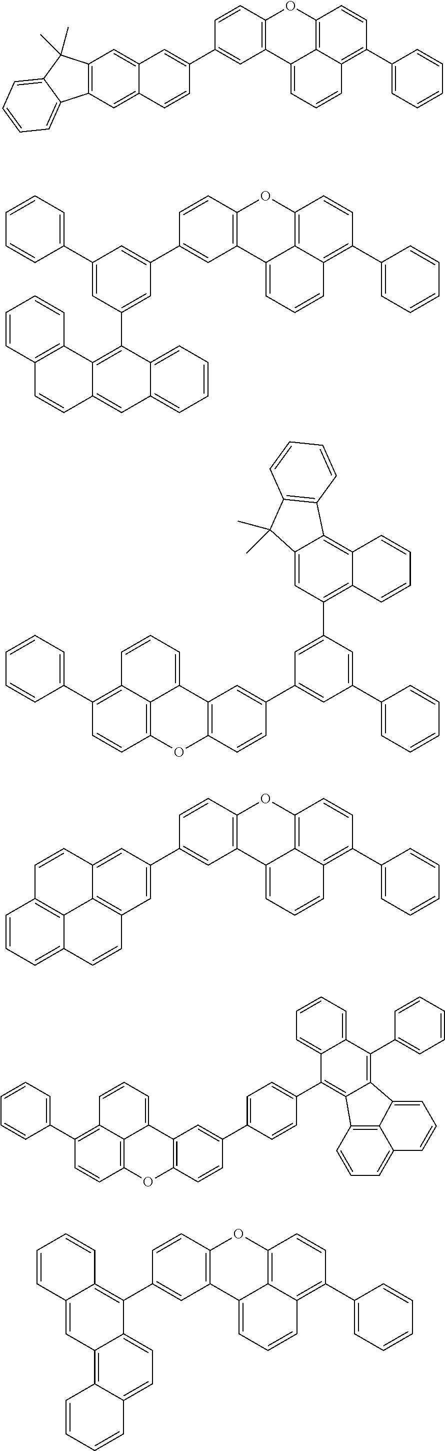

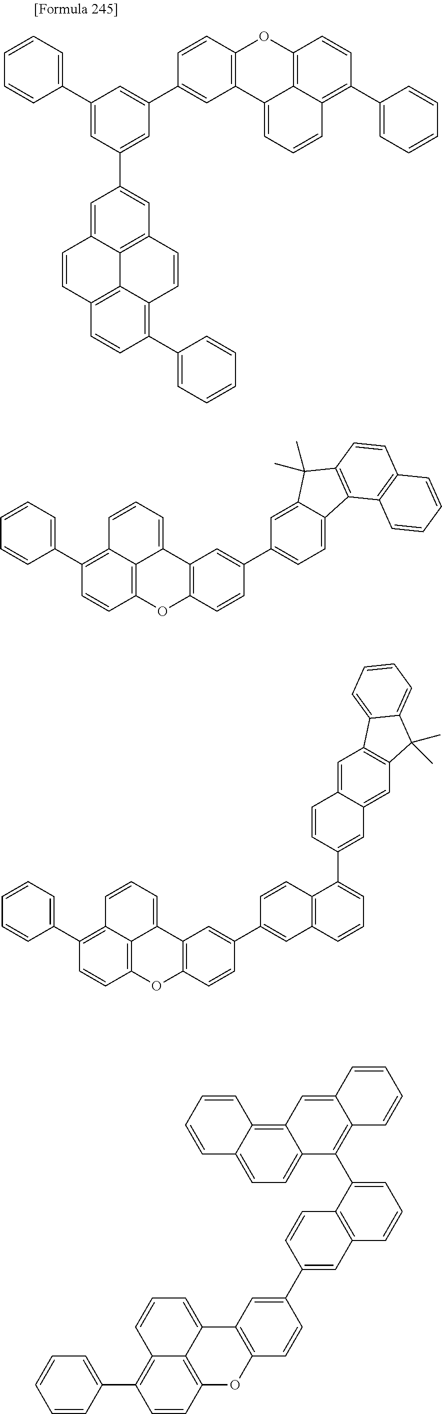 [SMILES: c1ccc(-c2cc(-c3ccc4c(c3)-c3cccc5c(-c6ccccc6)ccc(c35)O4)cc(-c3cc4ccc5ccc(-c6ccccc6)c6ccc(c3)c4c56)c2)cc1 ; CC1(C)c2cc(-c3ccc4c(c3)-c3cccc5c(-c6ccccc6)ccc(c35)O4)ccc2-c2c1ccc1ccccc21 ; CC1(C)c2ccccc2-c2cc3ccc(-c4cccc5cc(-c6ccc7c(c6)-c6cccc8c(-c9ccccc9)ccc(c68)O7)ccc45)cc3cc21 ; c1ccc(-c2ccc3c4c(cccc24)-c2cc(-c4ccc5c(-c6c7ccccc7cc7c6ccc6ccccc67)cccc5c4)ccc2O3)cc1]

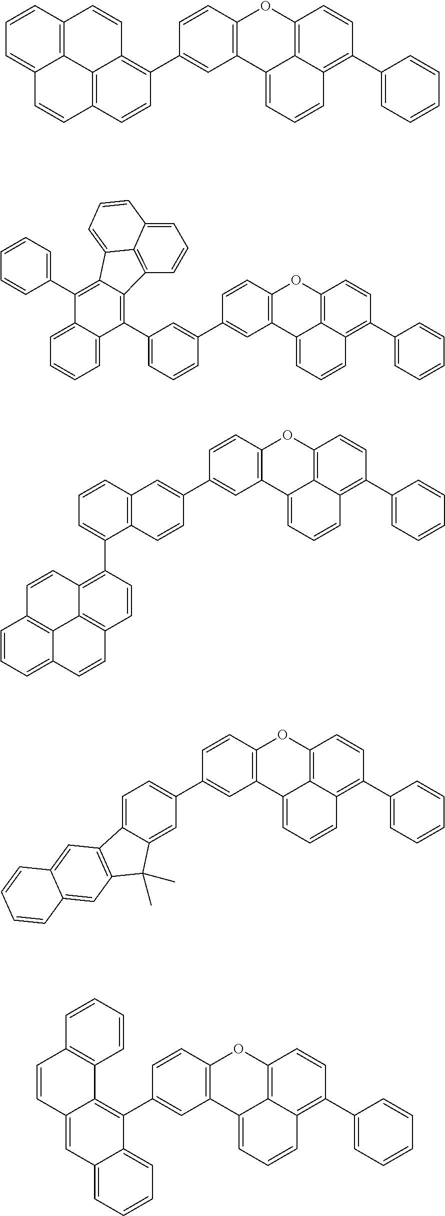 [SMILES: c1ccc(-c2ccc3c4c(cccc24)-c2cc(-c4ccc5ccc6cccc7ccc4c5c67)ccc2O3)cc1 ; c1ccc(-c2c3c(c(-c4cccc(-c5ccc6c(c5)-c5cccc7c(-c8ccccc8)ccc(c57)O6)c4)c4ccccc24)-c2cccc4cccc-3c24)cc1 ; c1ccc(-c2ccc3c4c(cccc24)-c2cc(-c4ccc5c(-c6ccc7ccc8cccc9ccc6c7c89)cccc5c4)ccc2O3)cc1 ; CC1(C)c2cc(-c3ccc4c(c3)-c3cccc5c(-c6ccccc6)ccc(c35)O4)ccc2-c2cc3ccccc3cc21 ; c1ccc(-c2ccc3c4c(cccc24)-c2cc(-c4c5ccccc5cc5ccc6ccccc6c45)ccc2O3)cc1]

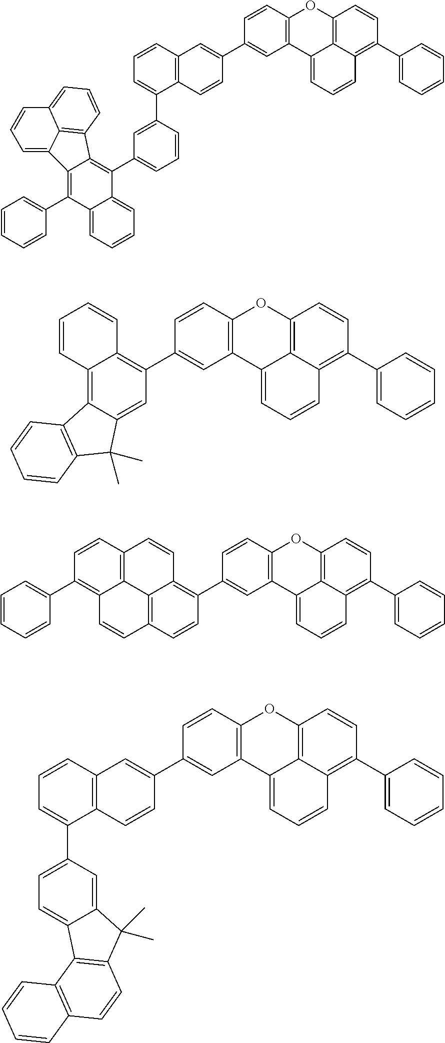 [SMILES: c1ccc(-c2c3c(c(-c4cccc(-c5cccc6cc(-c7ccc8c(c7)-c7cccc9c(-c%10ccccc%10)ccc(c79)O8)ccc56)c4)c4ccccc24)-c2cccc4cccc-3c24)cc1 ; CC1(C)c2ccccc2-c2c1cc(-c1ccc3c(c1)-c1cccc4c(-c5ccccc5)ccc(c14)O3)c1ccccc21 ; c1ccc(-c2ccc3c4c(cccc24)-c2cc(-c4ccc5ccc6c(-c7ccccc7)ccc7ccc4c5c76)ccc2O3)cc1 ; CC1(C)c2cc(-c3cccc4cc(-c5ccc6c(c5)-c5cccc7c(-c8ccccc8)ccc(c57)O6)ccc34)ccc2-c2c1ccc1ccccc21]

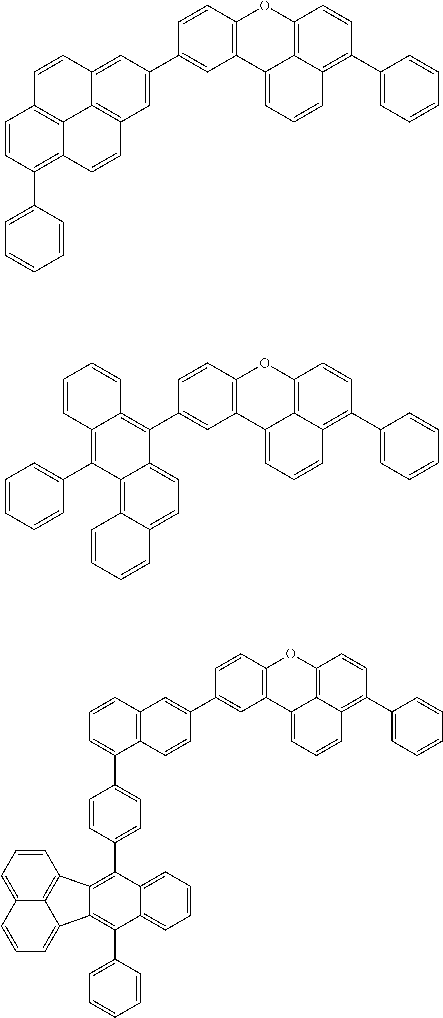 [SMILES: c1ccc(-c2ccc3c4c(cccc24)-c2cc(-c4cc5ccc6ccc(-c7ccccc7)c7ccc(c4)c5c67)ccc2O3)cc1 ; c1ccc(-c2ccc3c4c(cccc24)-c2cc(-c4c5ccccc5c(-c5ccccc5)c5c4ccc4ccccc45)ccc2O3)cc1 ; c1ccc(-c2c3c(c(-c4ccc(-c5cccc6cc(-c7ccc8c(c7)-c7cccc9c(-c%10ccccc%10)ccc(c79)O8)ccc56)cc4)c4ccccc24)-c2cccc4cccc-3c24)cc1]

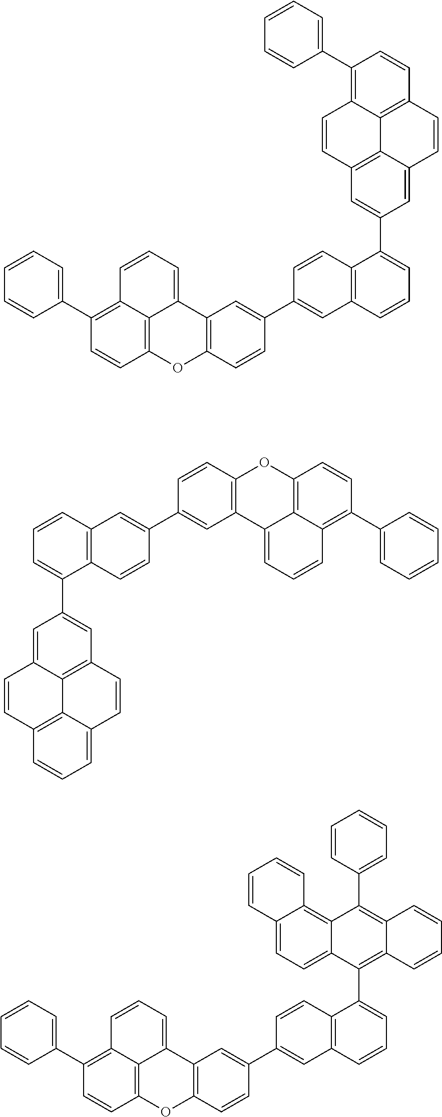 [SMILES: c1ccc(-c2ccc3c4c(cccc24)-c2cc(-c4ccc5c(-c6cc7ccc8ccc(-c9ccccc9)c9ccc(c6)c7c89)cccc5c4)ccc2O3)cc1 ; c1ccc(-c2ccc3c4c(cccc24)-c2cc(-c4ccc5c(-c6cc7ccc8cccc9ccc(c6)c7c89)cccc5c4)ccc2O3)cc1 ; c1ccc(-c2ccc3c4c(cccc24)-c2cc(-c4ccc5c(-c6c7ccccc7c(-c7ccccc7)c7c6ccc6ccccc67)cccc5c4)ccc2O3)cc1]

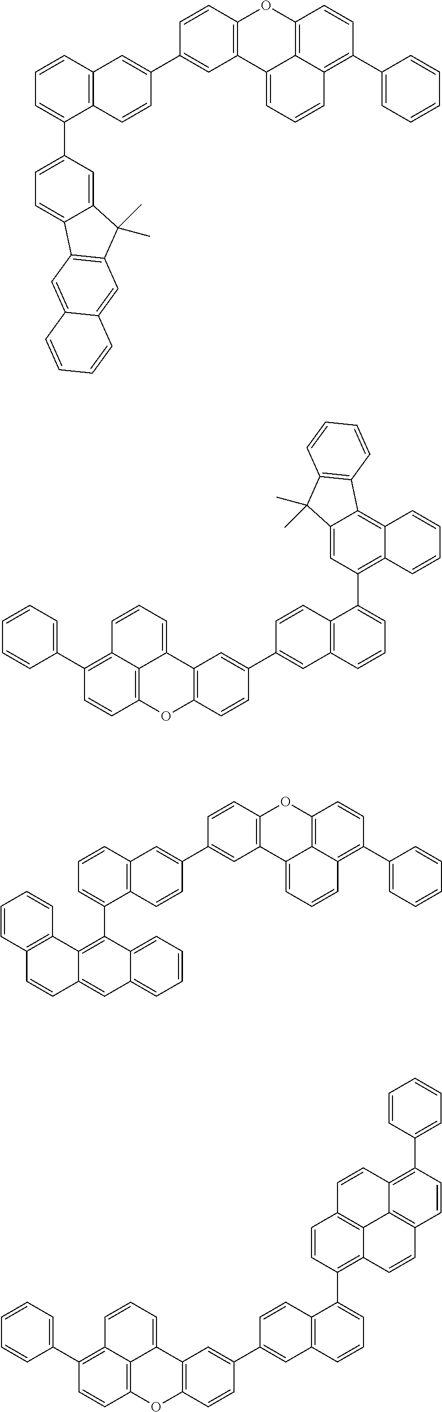 [SMILES: CC1(C)c2cc(-c3cccc4cc(-c5ccc6c(c5)-c5cccc7c(-c8ccccc8)ccc(c57)O6)ccc34)ccc2-c2cc3ccccc3cc21 ; CC1(C)c2ccccc2-c2c1cc(-c1cccc3cc(-c4ccc5c(c4)-c4cccc6c(-c7ccccc7)ccc(c46)O5)ccc13)c1ccccc21 ; c1ccc(-c2ccc3c4c(cccc24)-c2cc(-c4ccc5c(-c6c7ccccc7cc7ccc8ccccc8c67)cccc5c4)ccc2O3)cc1 ; c1ccc(-c2ccc3c4c(cccc24)-c2cc(-c4ccc5c(-c6ccc7ccc8c(-c9ccccc9)ccc9ccc6c7c98)cccc5c4)ccc2O3)cc1]

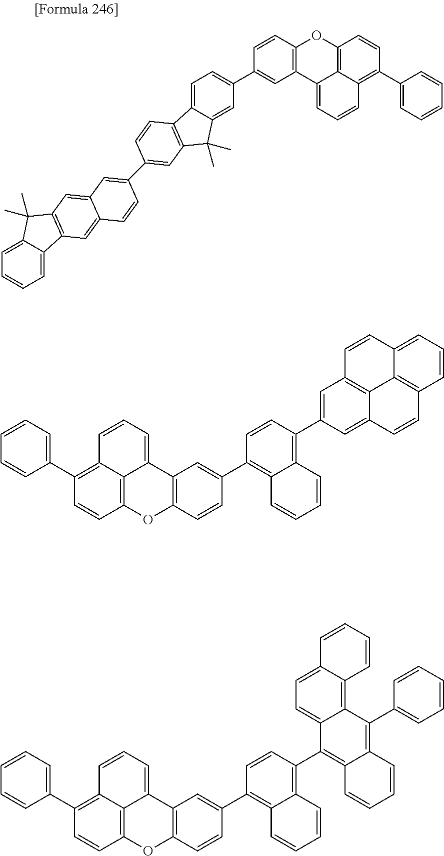 [SMILES: CC1(C)c2cc(-c3ccc4c(c3)-c3cccc5c(-c6ccccc6)ccc(c35)O4)ccc2-c2ccc(-c3ccc4cc5c(cc4c3)C(C)(C)c3ccccc3-5)cc21 ; c1ccc(-c2ccc3c4c(cccc24)-c2cc(-c4ccc(-c5cc6ccc7cccc8ccc(c5)c6c78)c5ccccc45)ccc2O3)cc1 ; c1ccc(-c2ccc3c4c(cccc24)-c2cc(-c4ccc(-c5c6ccccc6c(-c6ccccc6)c6c5ccc5ccccc56)c5ccccc45)ccc2O3)cc1]

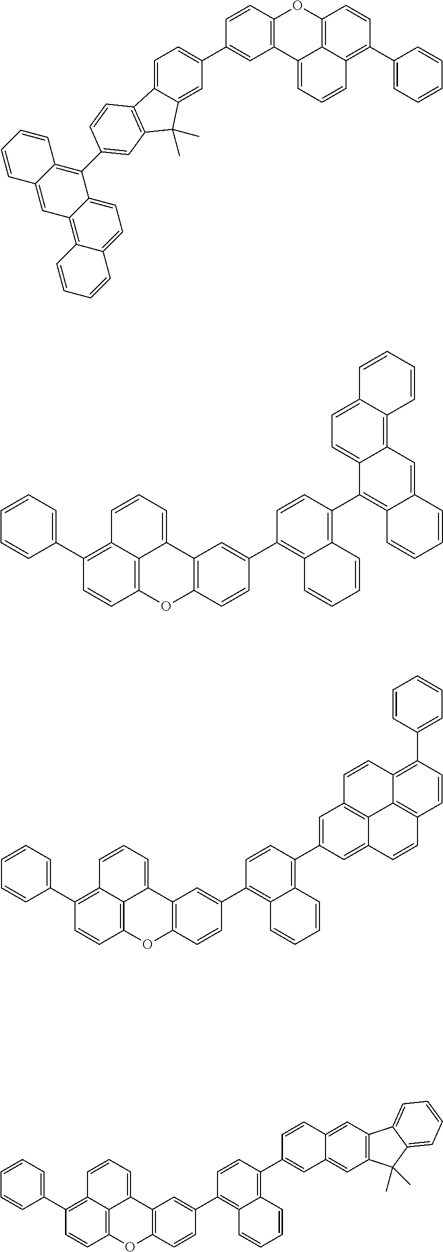 [SMILES: CC1(C)c2cc(-c3ccc4c(c3)-c3cccc5c(-c6ccccc6)ccc(c35)O4)ccc2-c2ccc(-c3c4ccccc4cc4c3ccc3ccccc34)cc21 ; c1ccc(-c2ccc3c4c(cccc24)-c2cc(-c4ccc(-c5c6ccccc6cc6c5ccc5ccccc56)c5ccccc45)ccc2O3)cc1 ; c1ccc(-c2ccc3c4c(cccc24)-c2cc(-c4ccc(-c5cc6ccc7ccc(-c8ccccc8)c8ccc(c5)c6c78)c5ccccc45)ccc2O3)cc1 ; CC1(C)c2ccccc2-c2cc3ccc(-c4ccc(-c5ccc6c(c5)-c5cccc7c(-c8ccccc8)ccc(c57)O6)c5ccccc45)cc3cc21]

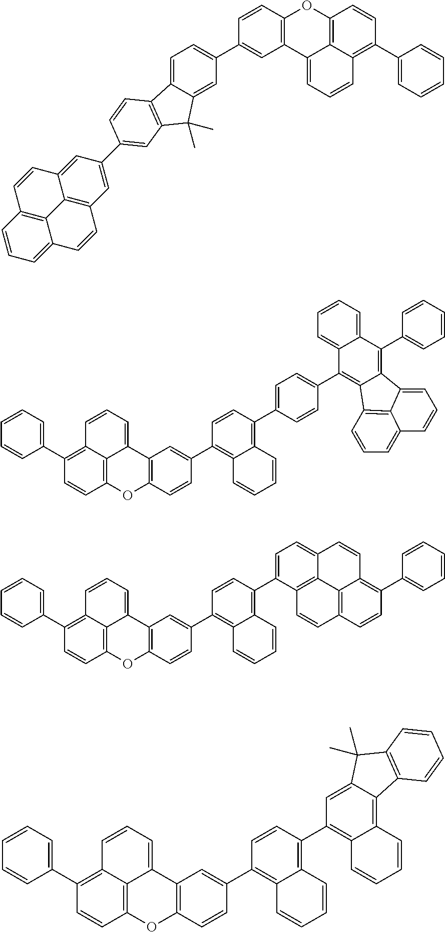 [SMILES: CC1(C)c2cc(-c3ccc4c(c3)-c3cccc5c(-c6ccccc6)ccc(c35)O4)ccc2-c2ccc(-c3cc4ccc5cccc6ccc(c3)c4c56)cc21 ; c1ccc(-c2c3c(c(-c4ccc(-c5ccc(-c6ccc7c(c6)-c6cccc8c(-c9ccccc9)ccc(c68)O7)c6ccccc56)cc4)c4ccccc24)-c2cccc4cccc-3c24)cc1 ; c1ccc(-c2ccc3c4c(cccc24)-c2cc(-c4ccc(-c5ccc6ccc7c(-c8ccccc8)ccc8ccc5c6c87)c5ccccc45)ccc2O3)cc1 ; CC1(C)c2ccccc2-c2c1cc(-c1ccc(-c3ccc4c(c3)-c3cccc5c(-c6ccccc6)ccc(c35)O4)c3ccccc13)c1ccccc21]

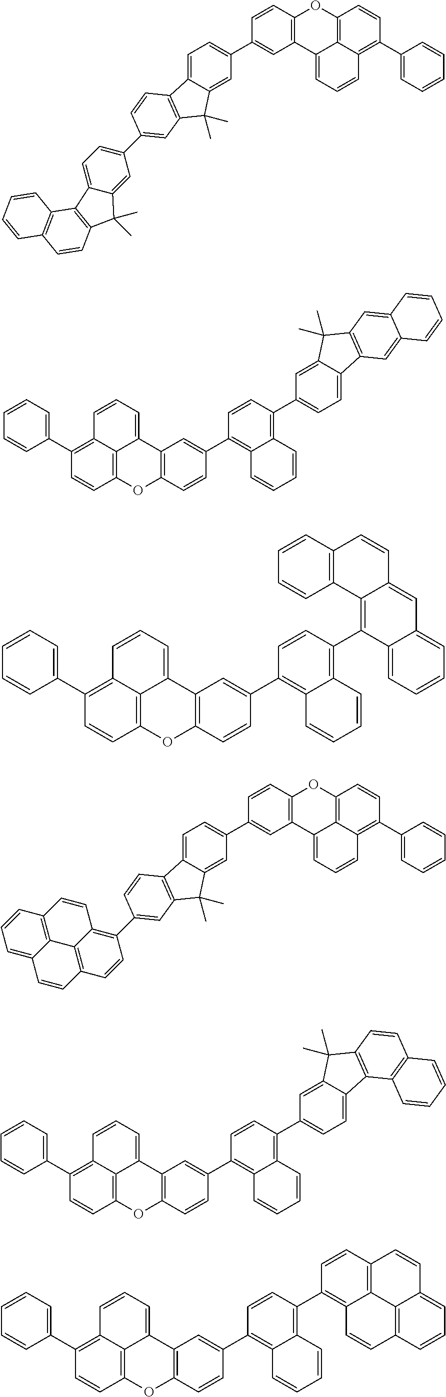

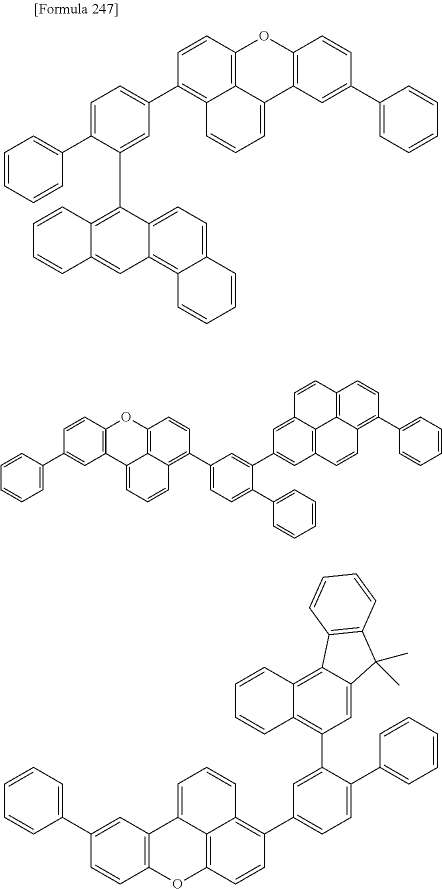 [SMILES: c1ccc(-c2ccc3c(c2)-c2cccc4c(-c5ccc(-c6ccccc6)c(-c6c7ccccc7cc7c6ccc6ccccc67)c5)ccc(c24)O3)cc1 ; c1ccc(-c2ccc3c(c2)-c2cccc4c(-c5ccc(-c6ccccc6)c(-c6cc7ccc8ccc(-c9ccccc9)c9ccc(c6)c7c89)c5)ccc(c24)O3)cc1 ; CC1(C)c2ccccc2-c2c1cc(-c1cc(-c3ccc4c5c(cccc35)-c3cc(-c5ccccc5)ccc3O4)ccc1-c1ccccc1)c1ccccc21]

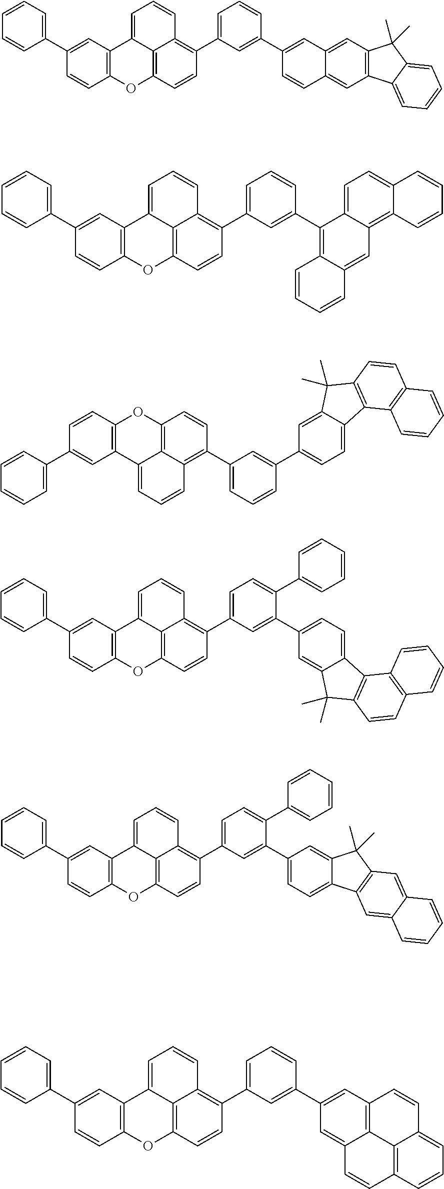

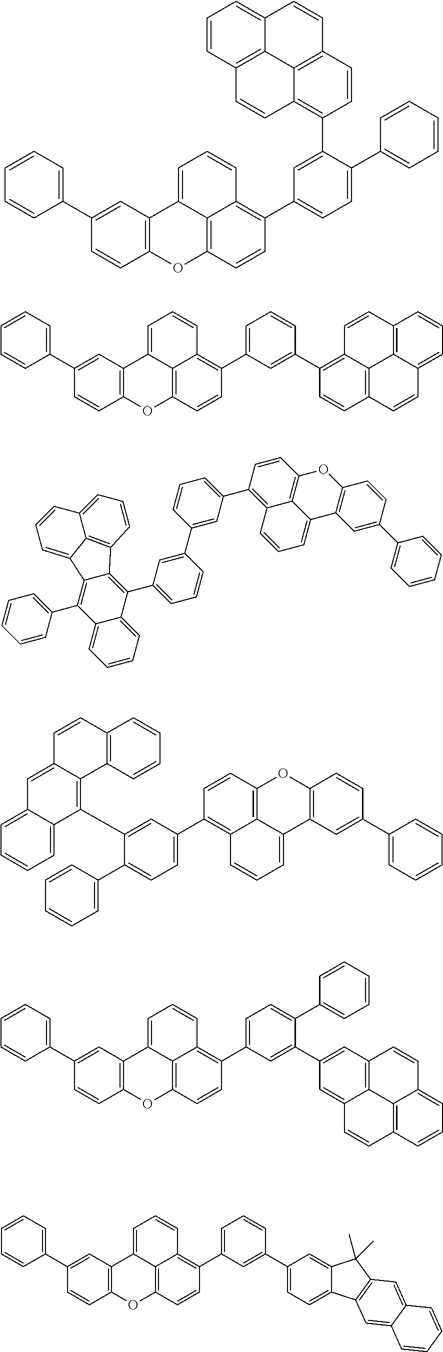

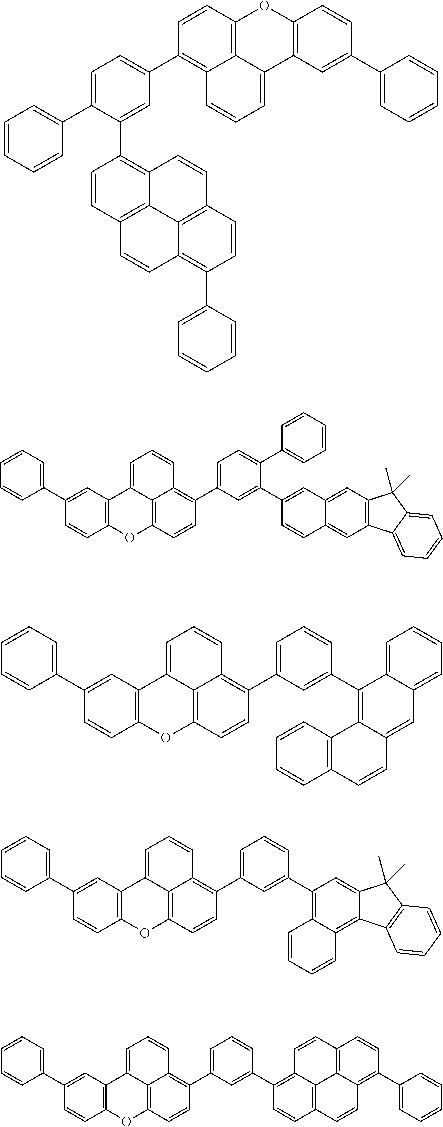 [SMILES: c1ccc(-c2ccc3c(c2)-c2cccc4c(-c5ccc(-c6ccccc6)c(-c6ccc7ccc8c(-c9ccccc9)ccc9ccc6c7c98)c5)ccc(c24)O3)cc1 ; CC1(C)c2ccccc2-c2cc3ccc(-c4cc(-c5ccc6c7c(cccc57)-c5cc(-c7ccccc7)ccc5O6)ccc4-c4ccccc4)cc3cc21 ; c1ccc(-c2ccc3c(c2)-c2cccc4c(-c5cccc(-c6c7ccccc7cc7ccc8ccccc8c67)c5)ccc(c24)O3)cc1 ; CC1(C)c2ccccc2-c2c1cc(-c1cccc(-c3ccc4c5c(cccc35)-c3cc(-c5ccccc5)ccc3O4)c1)c1ccccc21 ; c1ccc(-c2ccc3c(c2)-c2cccc4c(-c5cccc(-c6ccc7ccc8c(-c9ccccc9)ccc9ccc6c7c98)c5)ccc(c24)O3)cc1]

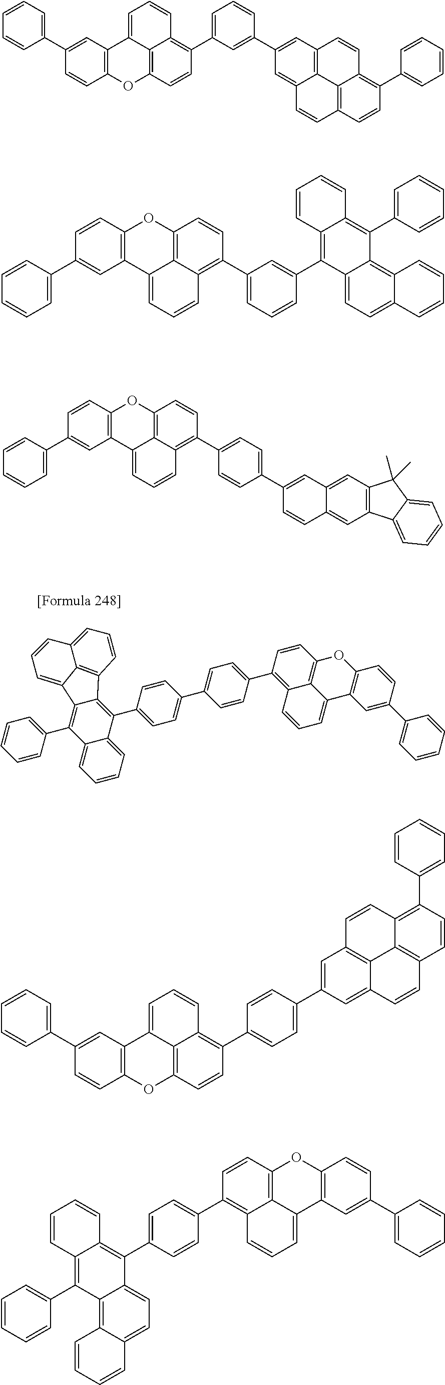

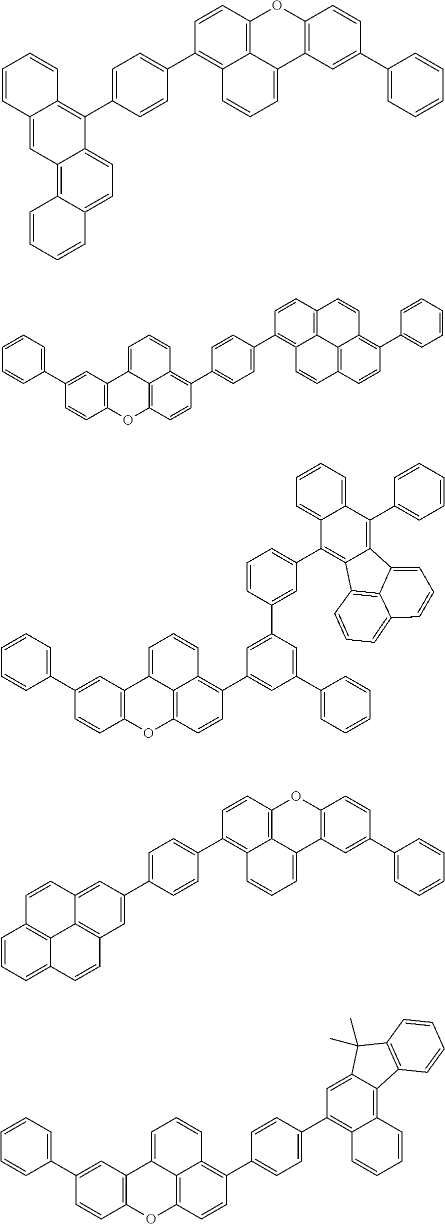 [SMILES: c1ccc(-c2ccc3c(c2)-c2cccc4c(-c5ccc(-c6c7ccccc7cc7c6ccc6ccccc67)cc5)ccc(c24)O3)cc1 ; c1ccc(-c2ccc3c(c2)-c2cccc4c(-c5ccc(-c6ccc7ccc8c(-c9ccccc9)ccc9ccc6c7c98)cc5)ccc(c24)O3)cc1 ; c1ccc(-c2cc(-c3cccc(-c4c5c(c(-c6ccccc6)c6ccccc46)-c4cccc6cccc-5c46)c3)cc(-c3ccc4c5c(cccc35)-c3cc(-c5ccccc5)ccc3O4)c2)cc1 ; c1ccc(-c2ccc3c(c2)-c2cccc4c(-c5ccc(-c6cc7ccc8cccc9ccc(c6)c7c89)cc5)ccc(c24)O3)cc1 ; CC1(C)c2ccccc2-c2c1cc(-c1ccc(-c3ccc4c5c(cccc35)-c3cc(-c5ccccc5)ccc3O4)cc1)c1ccccc21]

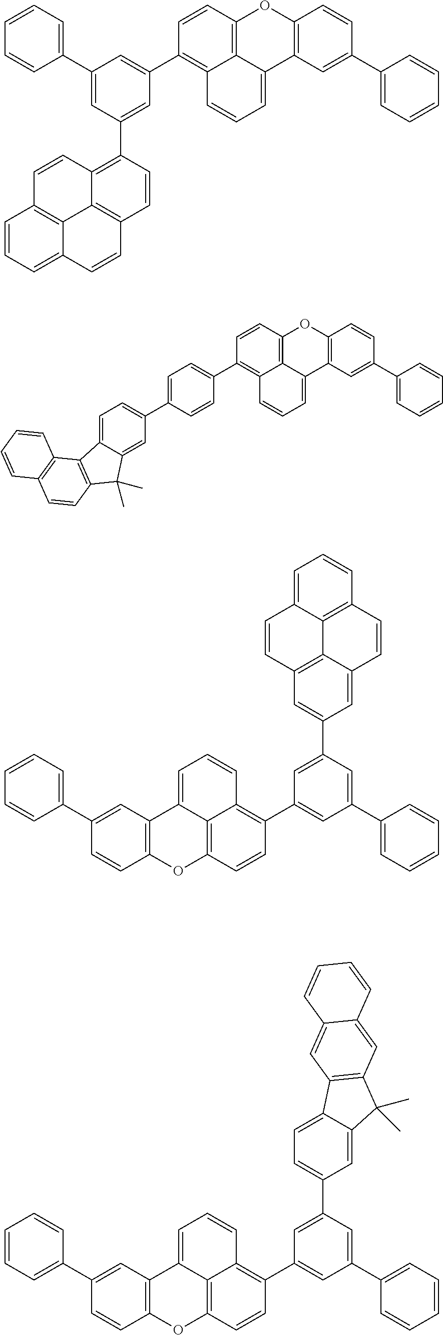 [SMILES: c1ccc(-c2cc(-c3ccc4c5c(cccc35)-c3cc(-c5ccccc5)ccc3O4)cc(-c3ccc4ccc5cccc6ccc3c4c56)c2)cc1 ; CC1(C)c2cc(-c3ccc(-c4ccc5c6c(cccc46)-c4cc(-c6ccccc6)ccc4O5)cc3)ccc2-c2c1ccc1ccccc21 ; c1ccc(-c2cc(-c3cc4ccc5cccc6ccc(c3)c4c56)cc(-c3ccc4c5c(cccc35)-c3cc(-c5ccccc5)ccc3O4)c2)cc1 ; CC1(C)c2cc(-c3cc(-c4ccccc4)cc(-c4ccc5c6c(cccc46)-c4cc(-c6ccccc6)ccc4O5)c3)ccc2-c2cc3ccccc3cc21]

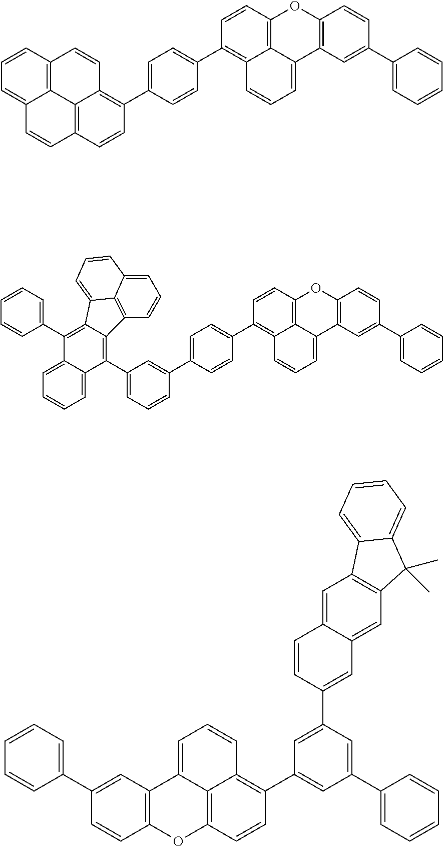 [SMILES: c1ccc(-c2ccc3c(c2)-c2cccc4c(-c5ccc(-c6ccc7ccc8cccc9ccc6c7c89)cc5)ccc(c24)O3)cc1 ; c1ccc(-c2ccc3c(c2)-c2cccc4c(-c5ccc(-c6cccc(-c7c8c(c(-c9ccccc9)c9ccccc79)-c7cccc9cccc-8c79)c6)cc5)ccc(c24)O3)cc1 ; CC1(C)c2ccccc2-c2cc3ccc(-c4cc(-c5ccccc5)cc(-c5ccc6c7c(cccc57)-c5cc(-c7ccccc7)ccc5O6)c4)cc3cc21]

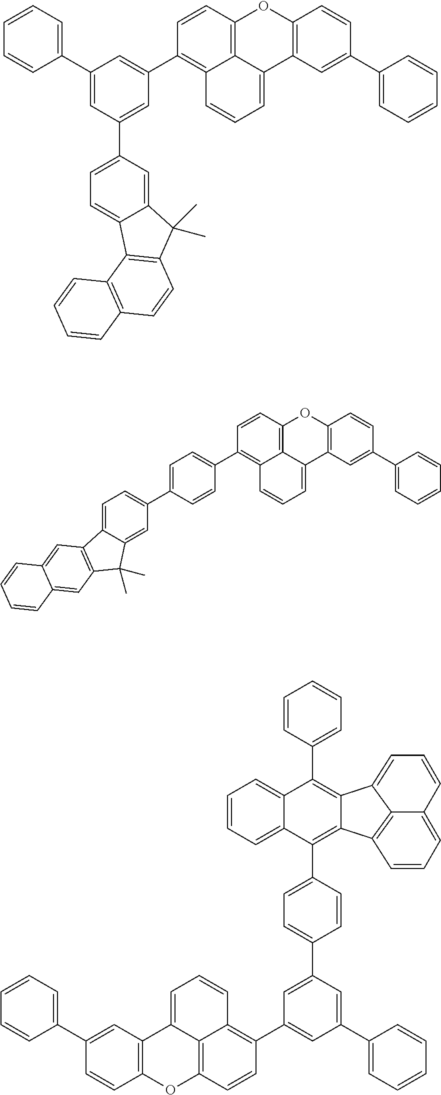 [SMILES: CC1(C)c2cc(-c3cc(-c4ccccc4)cc(-c4ccc5c6c(cccc46)-c4cc(-c6ccccc6)ccc4O5)c3)ccc2-c2c1ccc1ccccc21 ; CC1(C)c2cc(-c3ccc(-c4ccc5c6c(cccc46)-c4cc(-c6ccccc6)ccc4O5)cc3)ccc2-c2cc3ccccc3cc21 ; c1ccc(-c2cc(-c3ccc(-c4c5c(c(-c6ccccc6)c6ccccc46)-c4cccc6cccc-5c46)cc3)cc(-c3ccc4c5c(cccc35)-c3cc(-c5ccccc5)ccc3O4)c2)cc1]

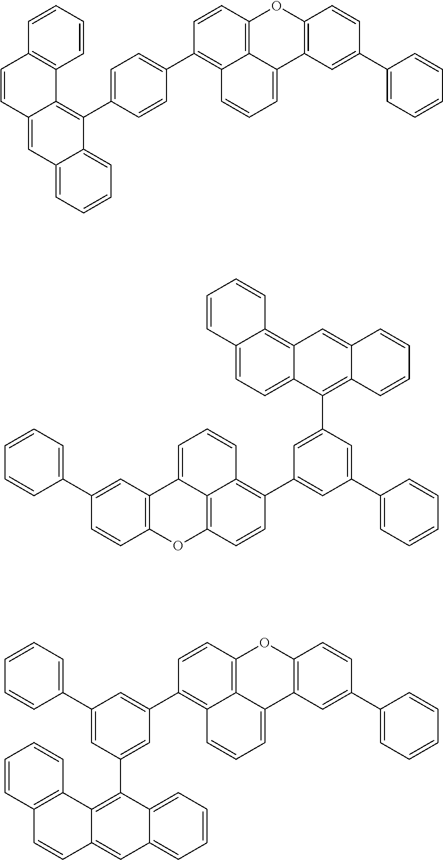 [SMILES: c1ccc(-c2ccc3c(c2)-c2cccc4c(-c5ccc(-c6c7ccccc7cc7ccc8ccccc8c67)cc5)ccc(c24)O3)cc1 ; c1ccc(-c2cc(-c3ccc4c5c(cccc35)-c3cc(-c5ccccc5)ccc3O4)cc(-c3c4ccccc4cc4c3ccc3ccccc34)c2)cc1 ; c1ccc(-c2cc(-c3ccc4c5c(cccc35)-c3cc(-c5ccccc5)ccc3O4)cc(-c3c4ccccc4cc4ccc5ccccc5c34)c2)cc1]

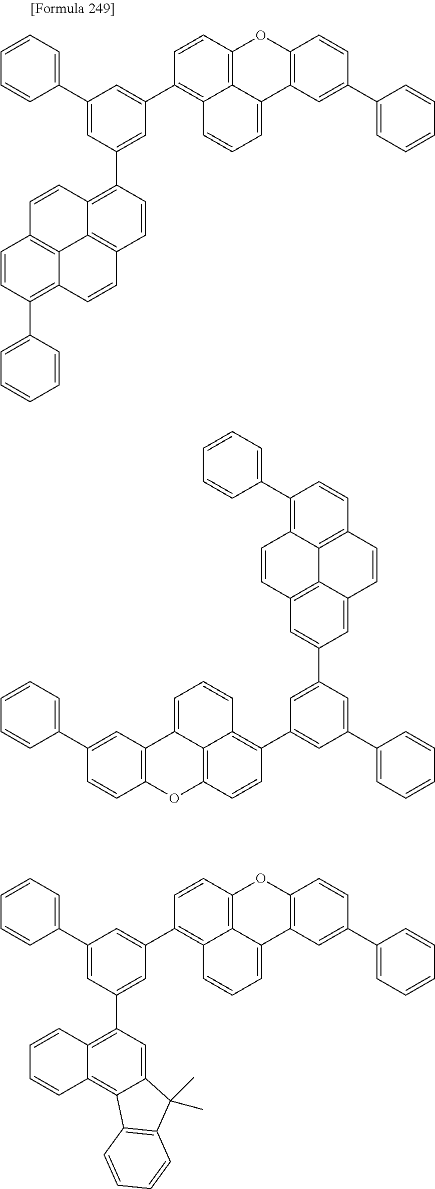 [SMILES: c1ccc(-c2cc(-c3ccc4c5c(cccc35)-c3cc(-c5ccccc5)ccc3O4)cc(-c3ccc4ccc5c(-c6ccccc6)ccc6ccc3c4c65)c2)cc1 ; c1ccc(-c2cc(-c3cc4ccc5ccc(-c6ccccc6)c6ccc(c3)c4c56)cc(-c3ccc4c5c(cccc35)-c3cc(-c5ccccc5)ccc3O4)c2)cc1 ; CC1(C)c2ccccc2-c2c1cc(-c1cc(-c3ccccc3)cc(-c3ccc4c5c(cccc35)-c3cc(-c5ccccc5)ccc3O4)c1)c1ccccc21]

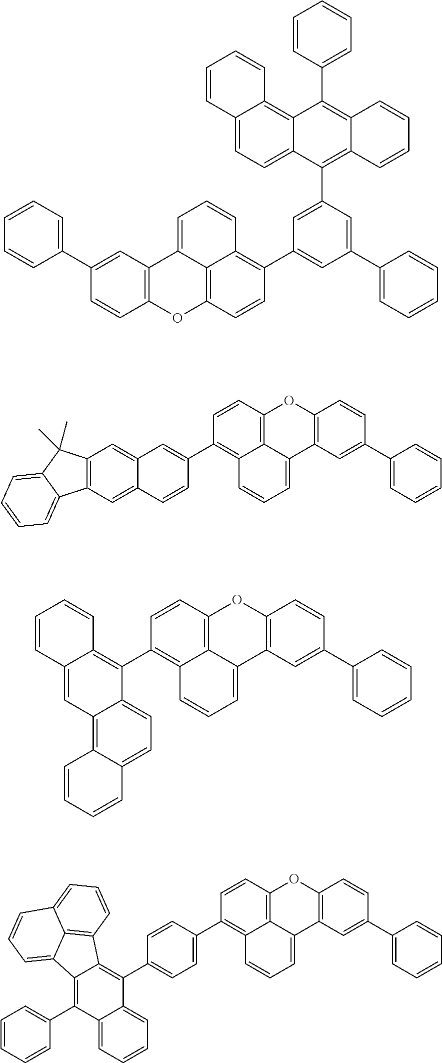 [SMILES: c1ccc(-c2cc(-c3ccc4c5c(cccc35)-c3cc(-c5ccccc5)ccc3O4)cc(-c3c4ccccc4c(-c4ccccc4)c4c3ccc3ccccc34)c2)cc1 ; CC1(C)c2ccccc2-c2cc3ccc(-c4ccc5c6c(cccc46)-c4cc(-c6ccccc6)ccc4O5)cc3cc21 ; c1ccc(-c2ccc3c(c2)-c2cccc4c(-c5c6ccccc6cc6c5ccc5ccccc56)ccc(c24)O3)cc1 ; c1ccc(-c2ccc3c(c2)-c2cccc4c(-c5ccc(-c6c7c(c(-c8ccccc8)c8ccccc68)-c6cccc8cccc-7c68)cc5)ccc(c24)O3)cc1]

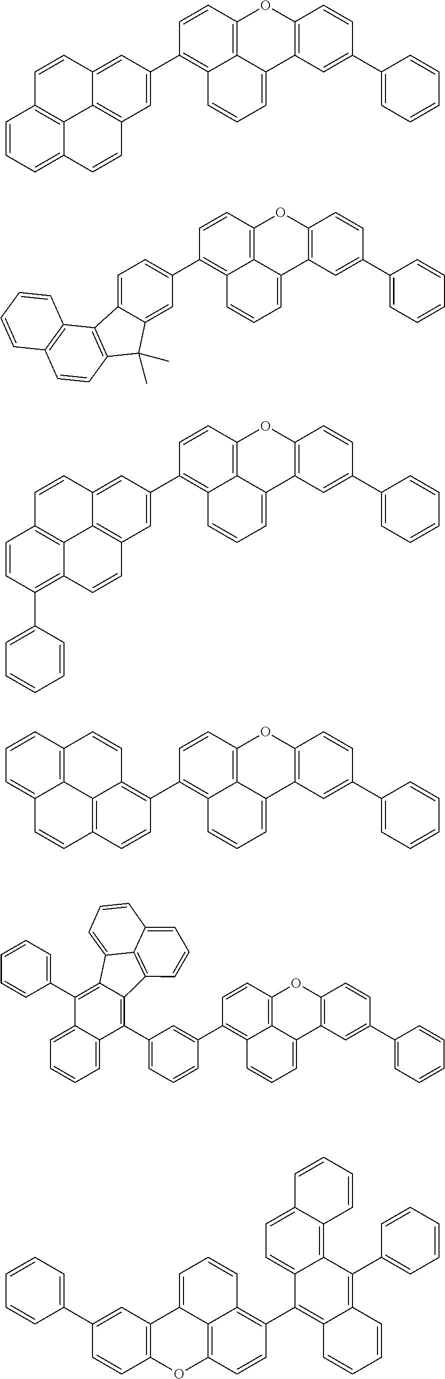

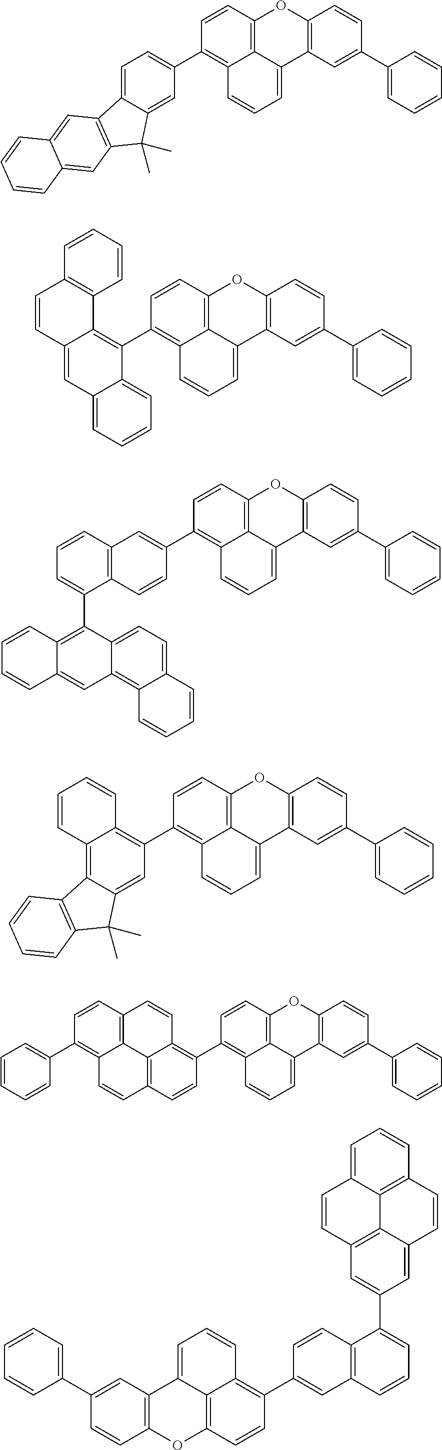 [SMILES: CC1(C)c2cc(-c3ccc4c5c(cccc35)-c3cc(-c5ccccc5)ccc3O4)ccc2-c2cc3ccccc3cc21 ; c1ccc(-c2ccc3c(c2)-c2cccc4c(-c5c6ccccc6cc6ccc7ccccc7c56)ccc(c24)O3)cc1 ; c1ccc(-c2ccc3c(c2)-c2cccc4c(-c5ccc6c(-c7c8ccccc8cc8c7ccc7ccccc78)cccc6c5)ccc(c24)O3)cc1 ; CC1(C)c2ccccc2-c2c1cc(-c1ccc3c4c(cccc14)-c1cc(-c4ccccc4)ccc1O3)c1ccccc21 ; c1ccc(-c2ccc3c(c2)-c2cccc4c(-c5ccc6ccc7c(-c8ccccc8)ccc8ccc5c6c87)ccc(c24)O3)cc1 ; c1ccc(-c2ccc3c(c2)-c2cccc4c(-c5ccc6c(-c7cc8ccc9cccc%10ccc(c7)c8c9%10)cccc6c5)ccc(c24)O3)cc1]

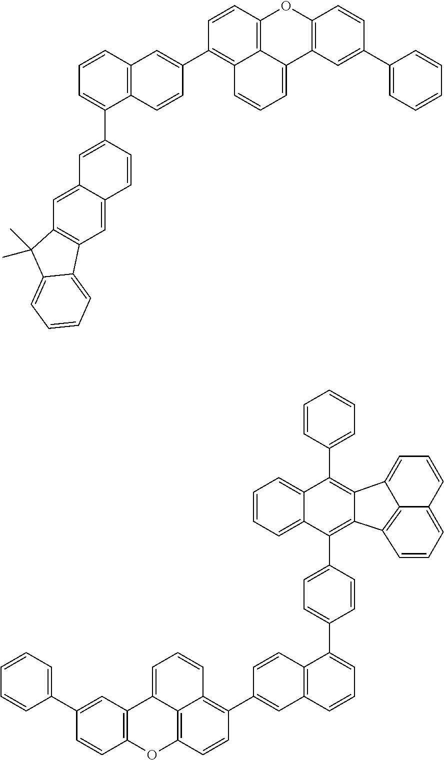 [SMILES: CC1(C)c2ccccc2-c2cc3ccc(-c4cccc5cc(-c6ccc7c8c(cccc68)-c6cc(-c8ccccc8)ccc6O7)ccc45)cc3cc21 ; c1ccc(-c2ccc3c(c2)-c2cccc4c(-c5ccc6c(-c7ccc(-c8c9c(c(-c%10ccccc%10)c%10ccccc8%10)-c8cccc%10cccc-9c8%10)cc7)cccc6c5)ccc(c24)O3)cc1]

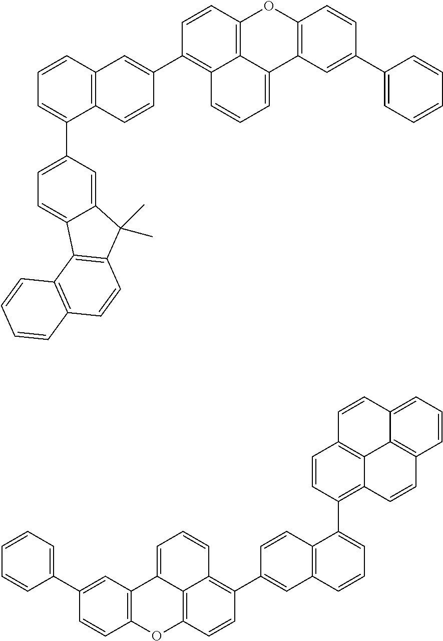 [SMILES: CC1(C)c2cc(-c3cccc4cc(-c5ccc6c7c(cccc57)-c5cc(-c7ccccc7)ccc5O6)ccc34)ccc2-c2c1ccc1ccccc21 ; c1ccc(-c2ccc3c(c2)-c2cccc4c(-c5ccc6c(-c7ccc8ccc9cccc%10ccc7c8c9%10)cccc6c5)ccc(c24)O3)cc1]

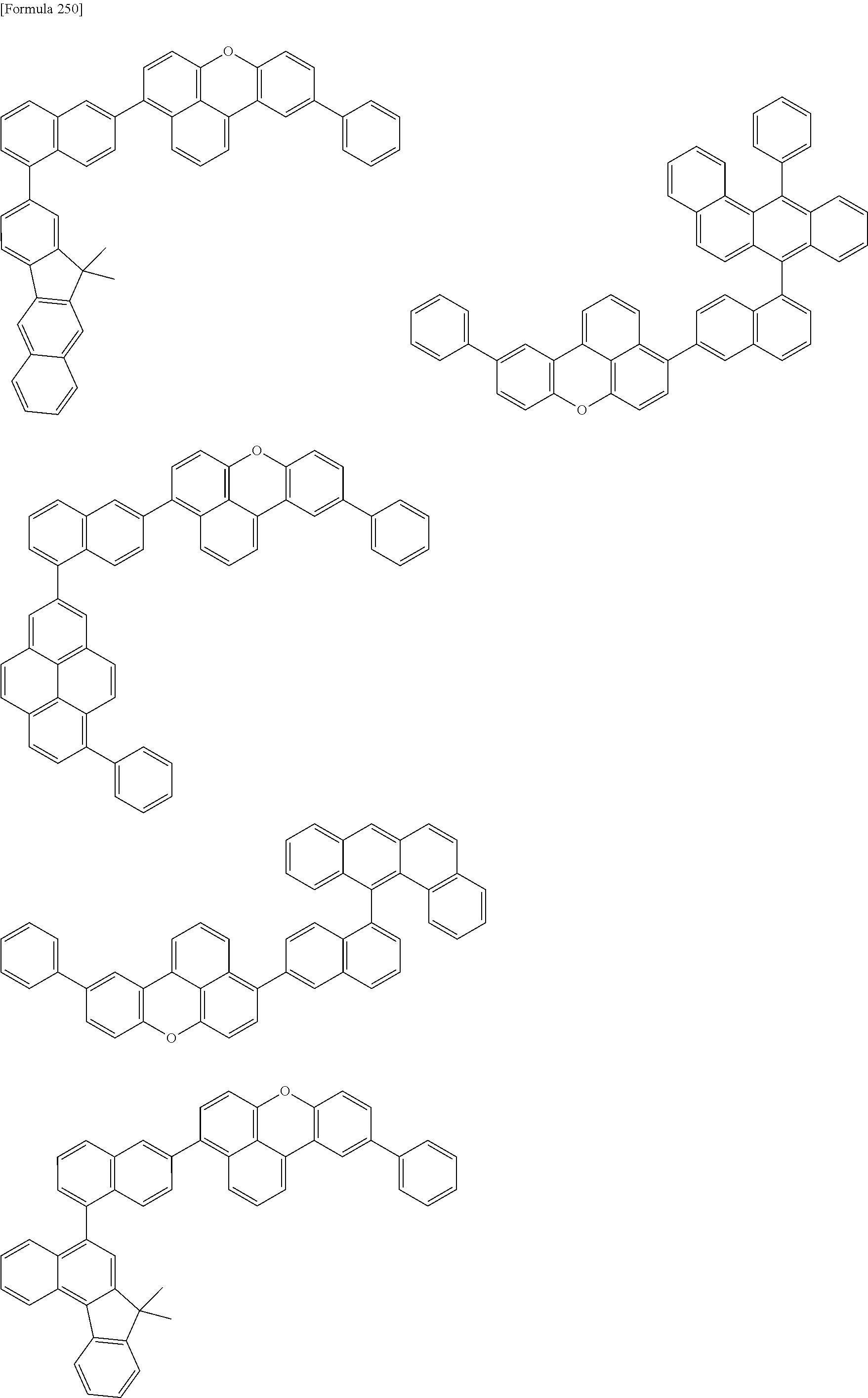 [SMILES: CC1(C)c2cc(-c3cccc4cc(-c5ccc6c7c(cccc57)-c5cc(-c7ccccc7)ccc5O6)ccc34)ccc2-c2cc3ccccc3cc21 ; c1ccc(-c2ccc3c(c2)-c2cccc4c(-c5ccc6c(-c7c8ccccc8c(-c8ccccc8)c8c7ccc7ccccc78)cccc6c5)ccc(c24)O3)cc1 ; c1ccc(-c2ccc3c(c2)-c2cccc4c(-c5ccc6c(-c7cc8ccc9ccc(-c%10ccccc%10)c%10ccc(c7)c8c9%10)cccc6c5)ccc(c24)O3)cc1 ; c1ccc(-c2ccc3c(c2)-c2cccc4c(-c5ccc6c(-c7c8ccccc8cc8ccc9ccccc9c78)cccc6c5)ccc(c24)O3)cc1 ; CC1(C)c2ccccc2-c2c1cc(-c1cccc3cc(-c4ccc5c6c(cccc46)-c4cc(-c6ccccc6)ccc4O5)ccc13)c1ccccc21]

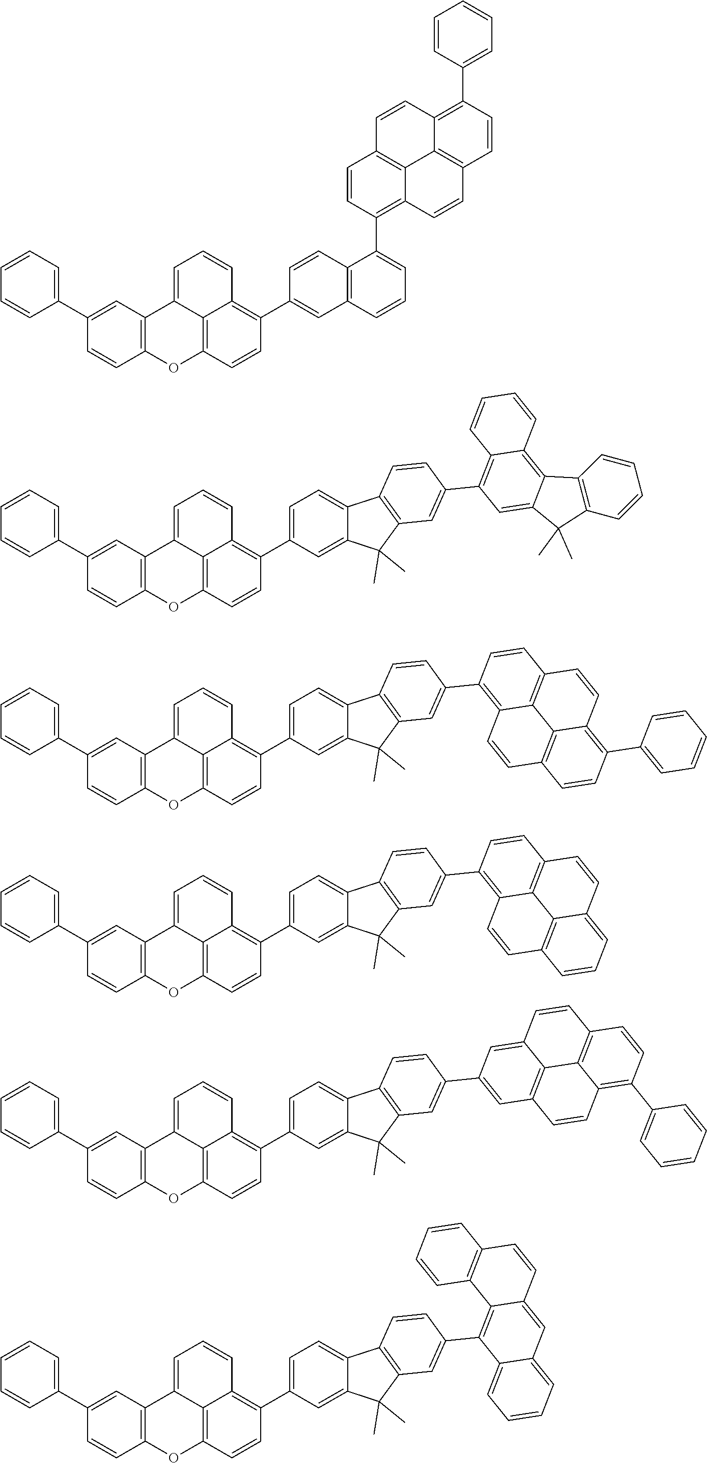

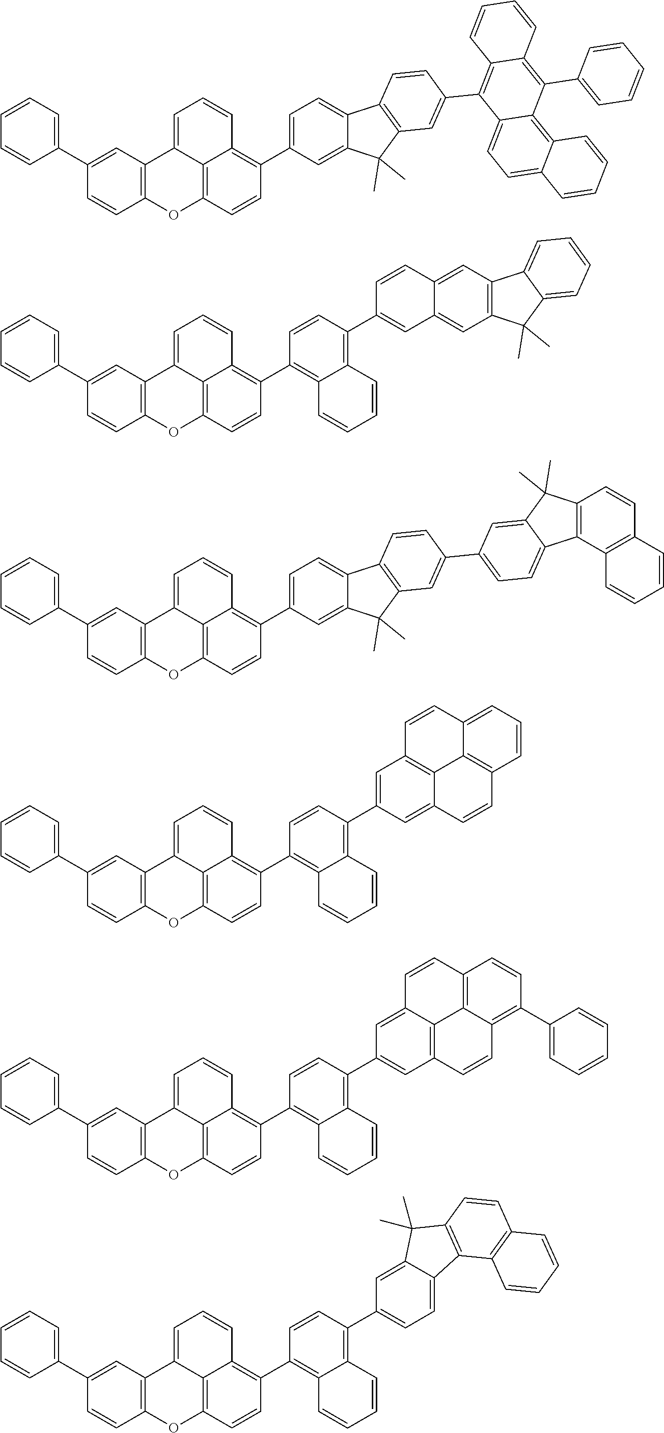

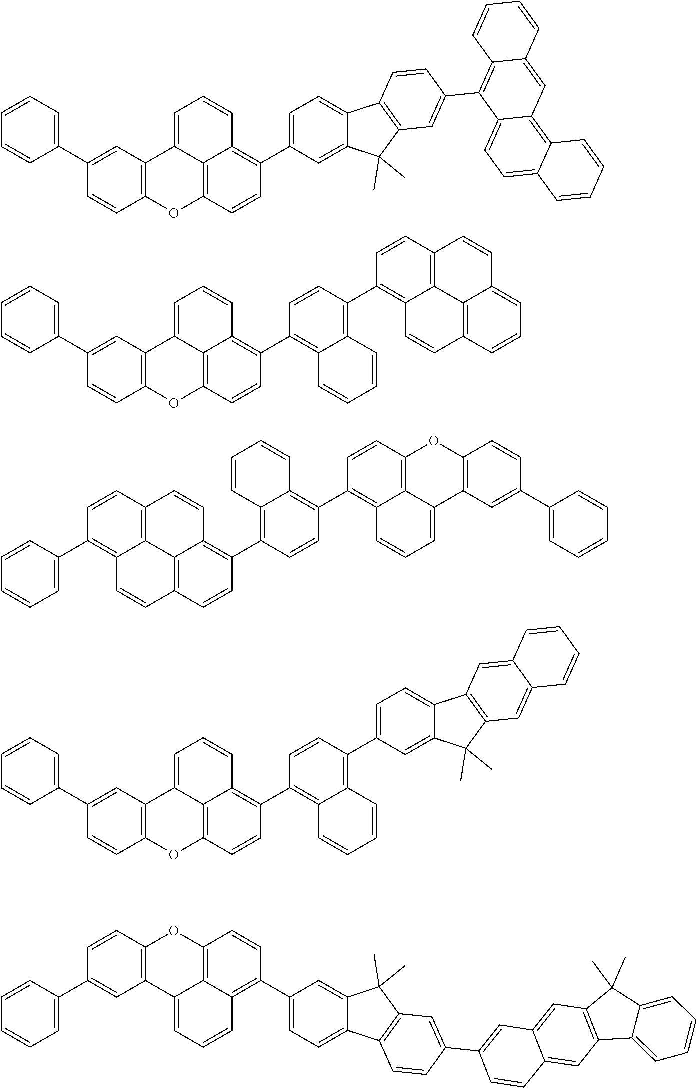 [SMILES: CC1(C)c2cc(-c3ccc4c5c(cccc35)-c3cc(-c5ccccc5)ccc3O4)ccc2-c2ccc(-c3c4ccccc4cc4c3ccc3ccccc34)cc21 ; c1ccc(-c2ccc3c(c2)-c2cccc4c(-c5ccc(-c6ccc7ccc8cccc9ccc6c7c89)c6ccccc56)ccc(c24)O3)cc1 ; c1ccc(-c2ccc3c(c2)-c2cccc4c(-c5ccc(-c6ccc7ccc8c(-c9ccccc9)ccc9ccc6c7c98)c6ccccc56)ccc(c24)O3)cc1 ; CC1(C)c2cc(-c3ccc(-c4ccc5c6c(cccc46)-c4cc(-c6ccccc6)ccc4O5)c4ccccc34)ccc2-c2cc3ccccc3cc21 ; CC1(C)c2cc(-c3ccc4cc5c(cc4c3)C(C)(C)c3ccccc3-5)ccc2-c2ccc(-c3ccc4c5c(cccc35)-c3cc(-c5ccccc5)ccc3O4)cc21]

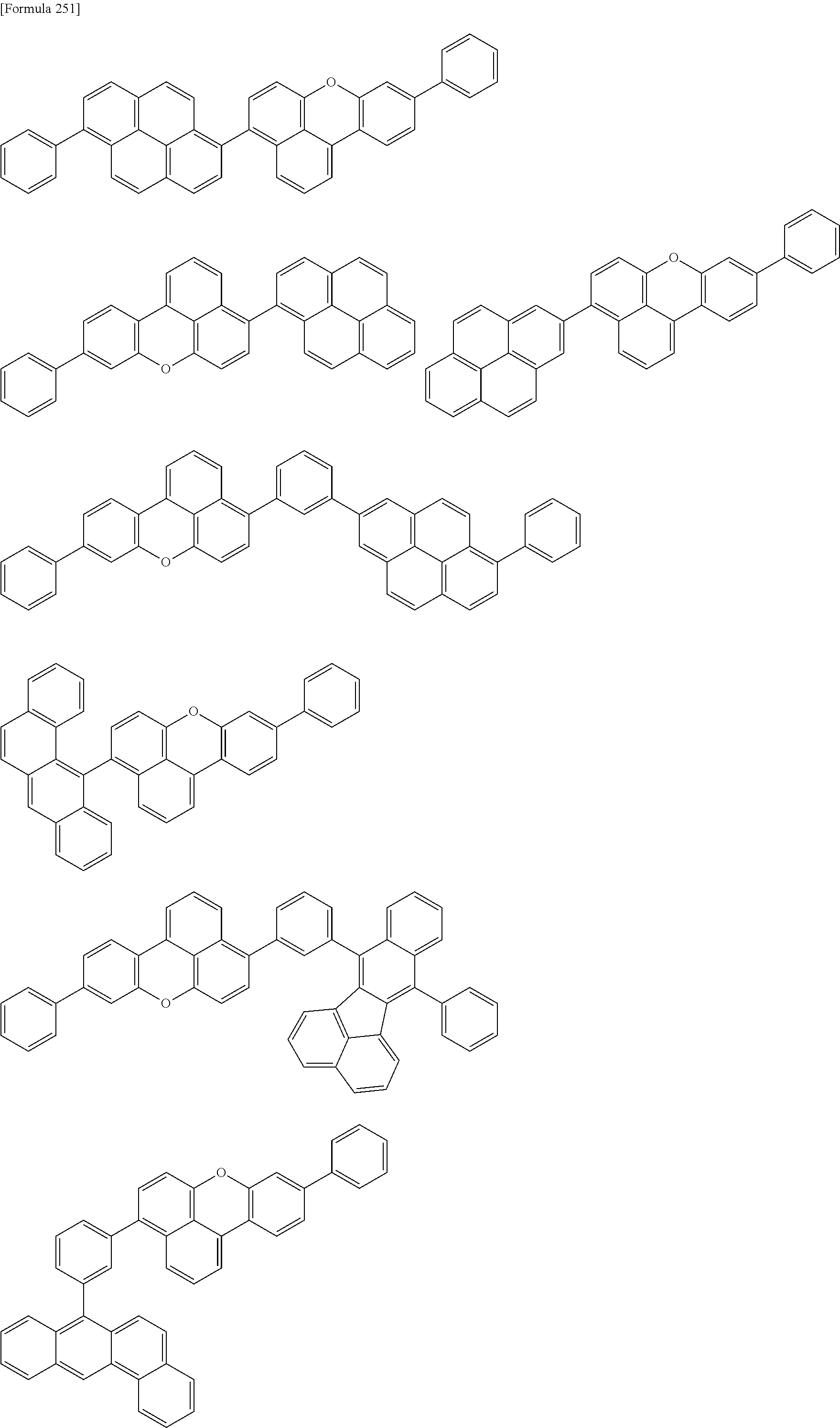

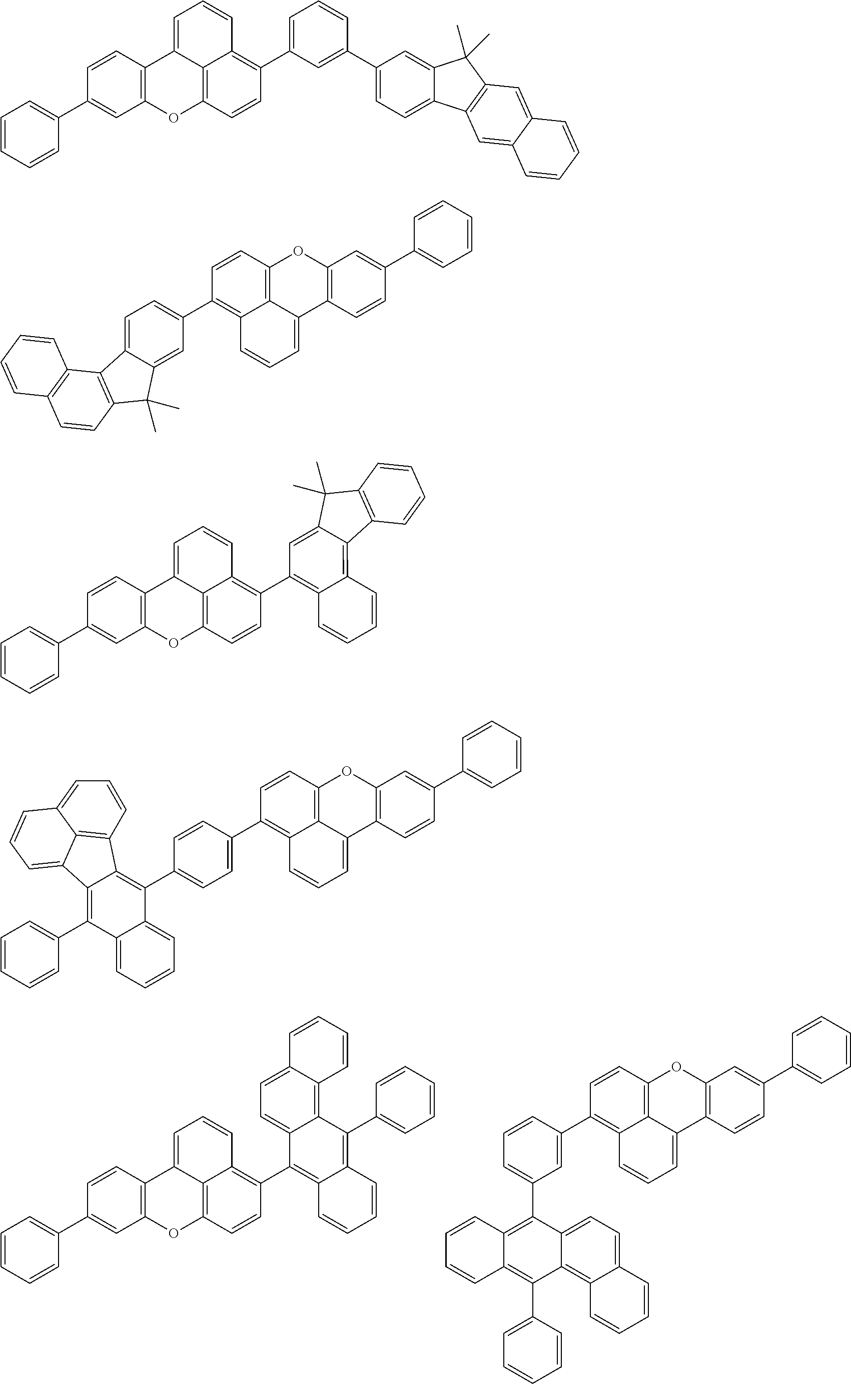

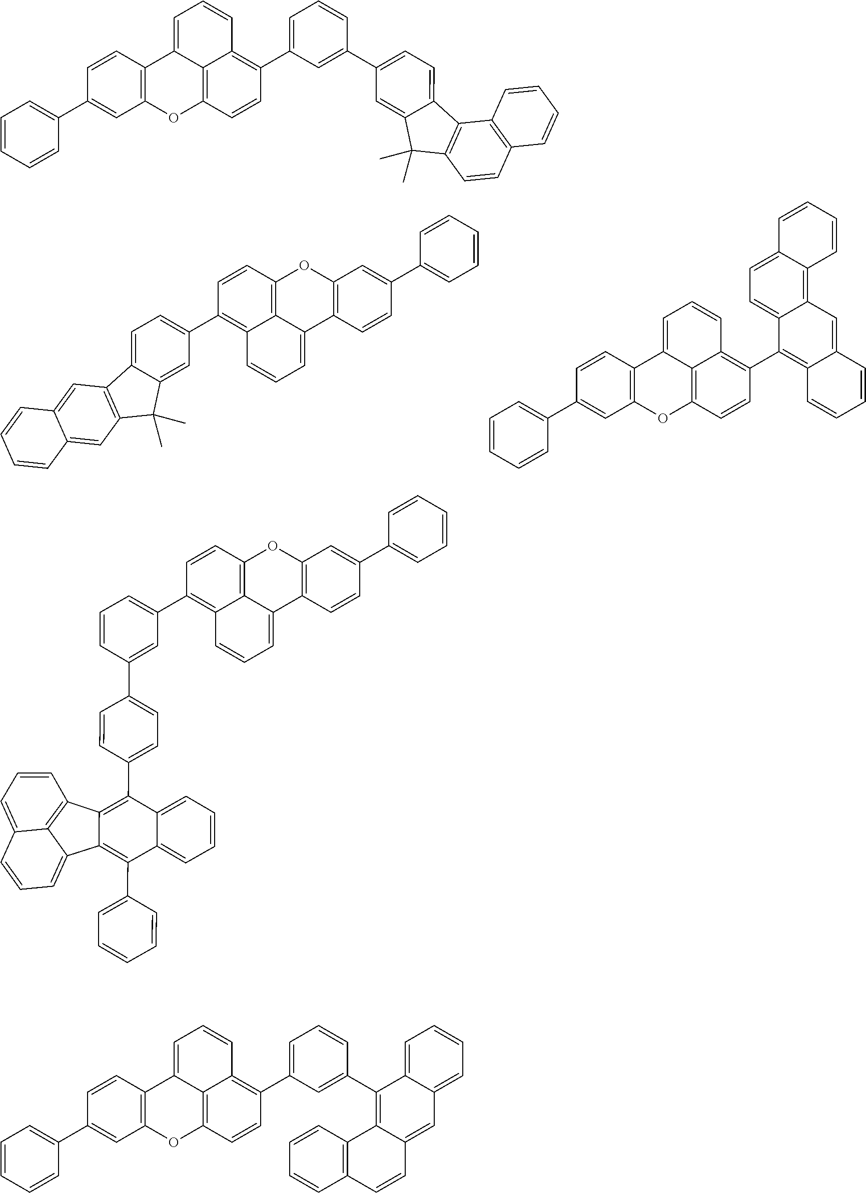

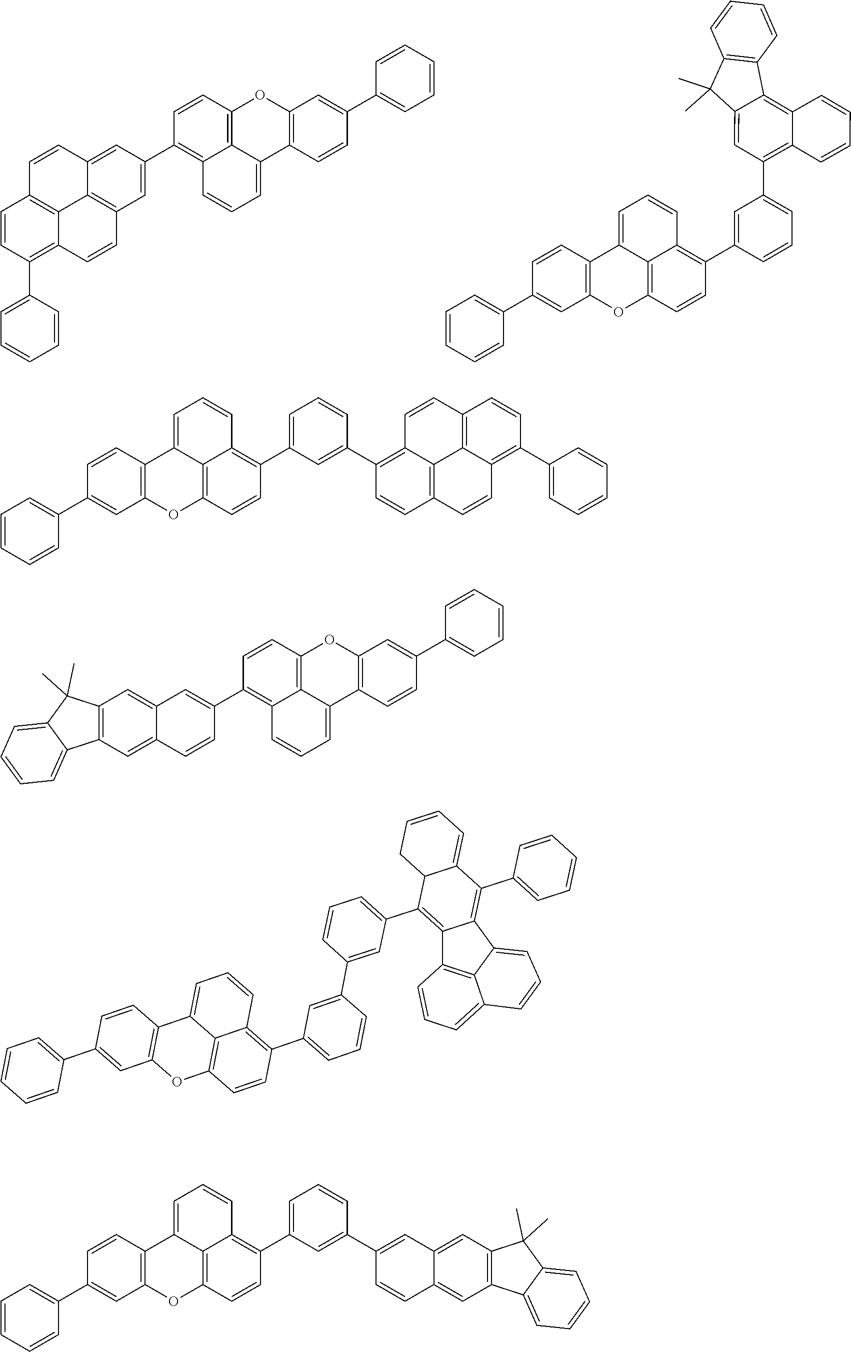

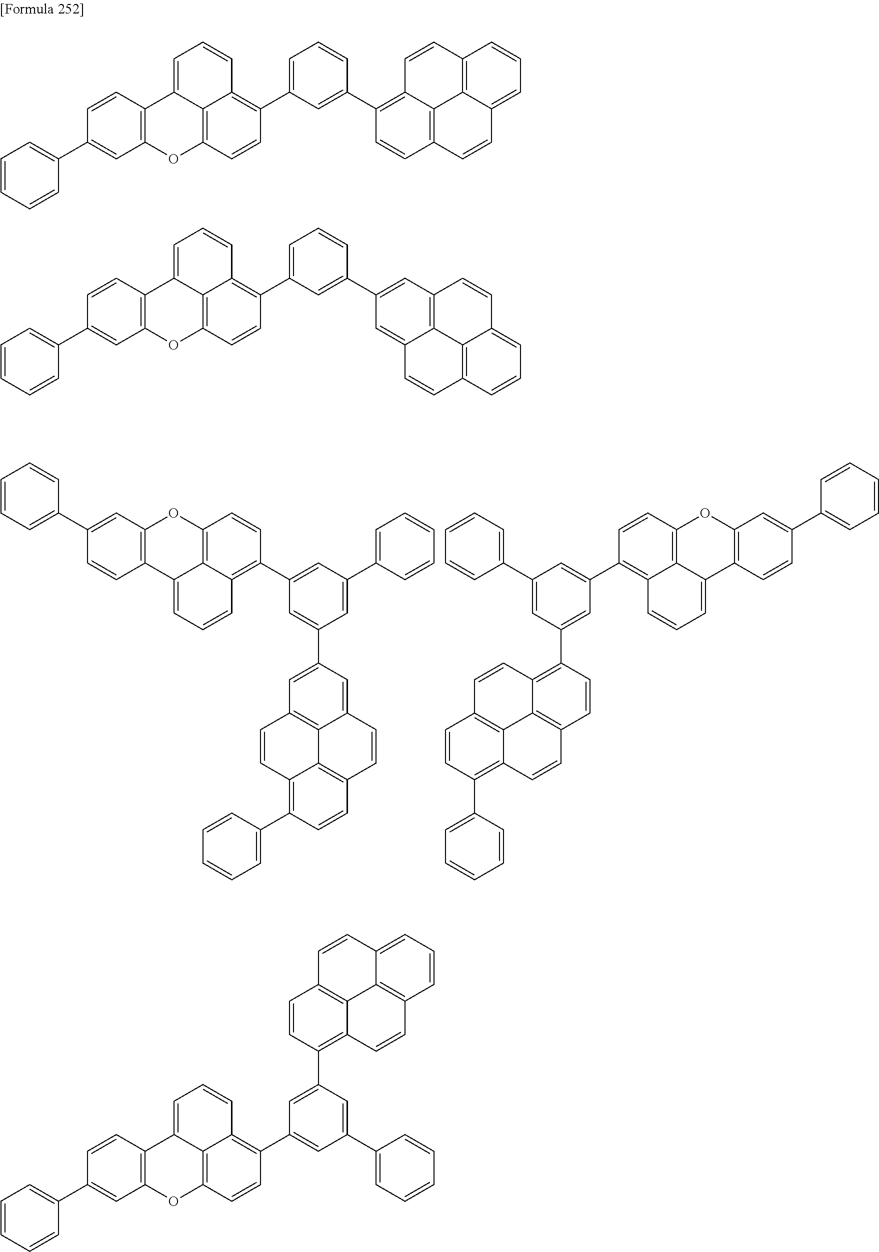

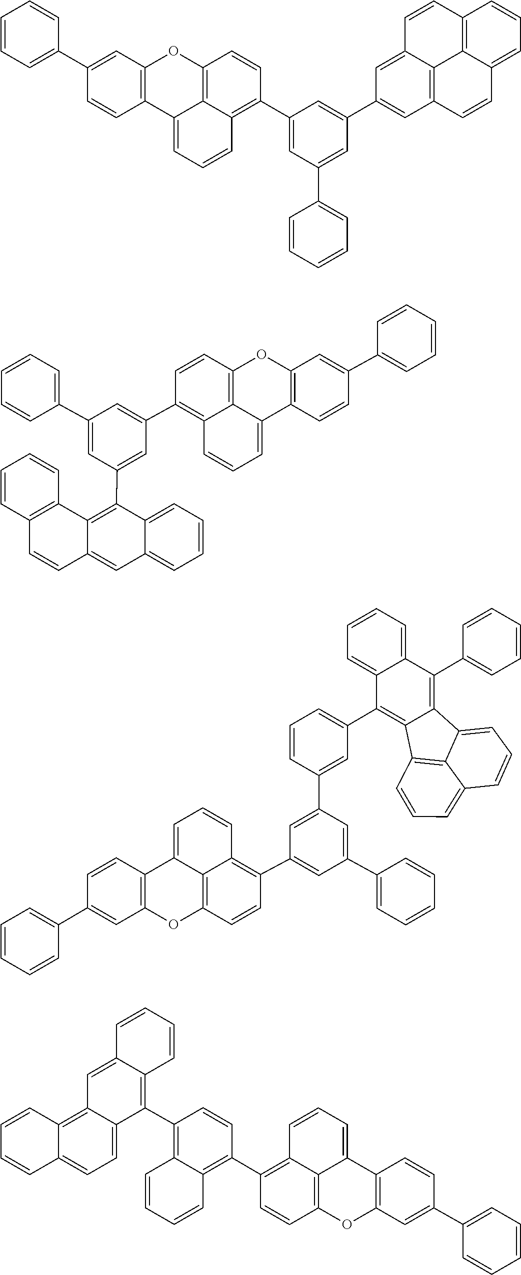 [SMILES: c1ccc(-c2cc(-c3cc4ccc5cccc6ccc(c3)c4c56)cc(-c3ccc4c5c(cccc35)-c3ccc(-c5ccccc5)cc3O4)c2)cc1 ; c1ccc(-c2cc(-c3ccc4c5c(cccc35)-c3ccc(-c5ccccc5)cc3O4)cc(-c3c4ccccc4cc4ccc5ccccc5c34)c2)cc1 ; c1ccc(-c2cc(-c3cccc(-c4c5c(c(-c6ccccc6)c6ccccc46)-c4cccc6cccc-5c46)c3)cc(-c3ccc4c5c(cccc35)-c3ccc(-c5ccccc5)cc3O4)c2)cc1 ; c1ccc(-c2ccc3c(c2)Oc2ccc(-c4ccc(-c5c6ccccc6cc6c5ccc5ccccc56)c5ccccc45)c4cccc-3c24)cc1]

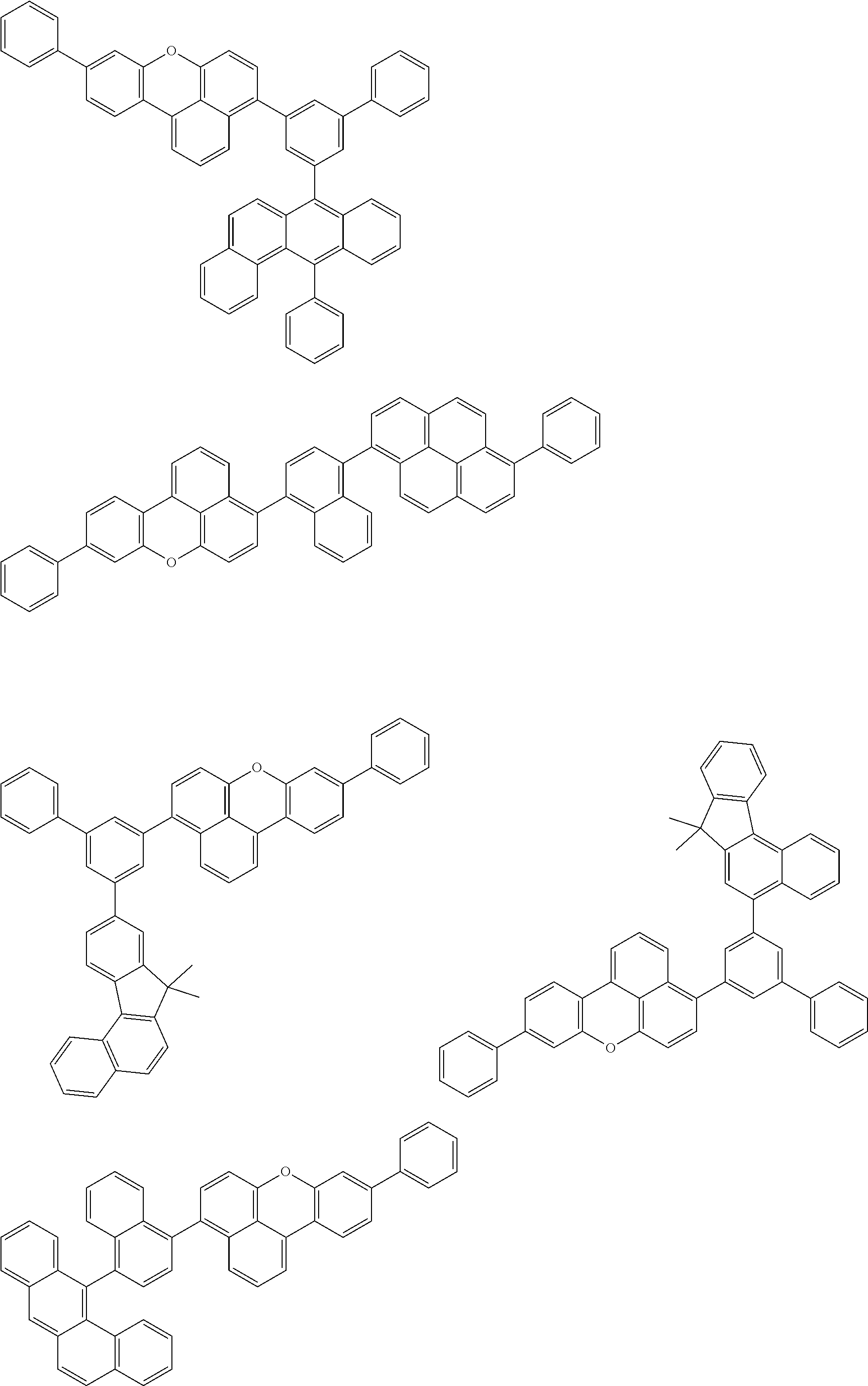

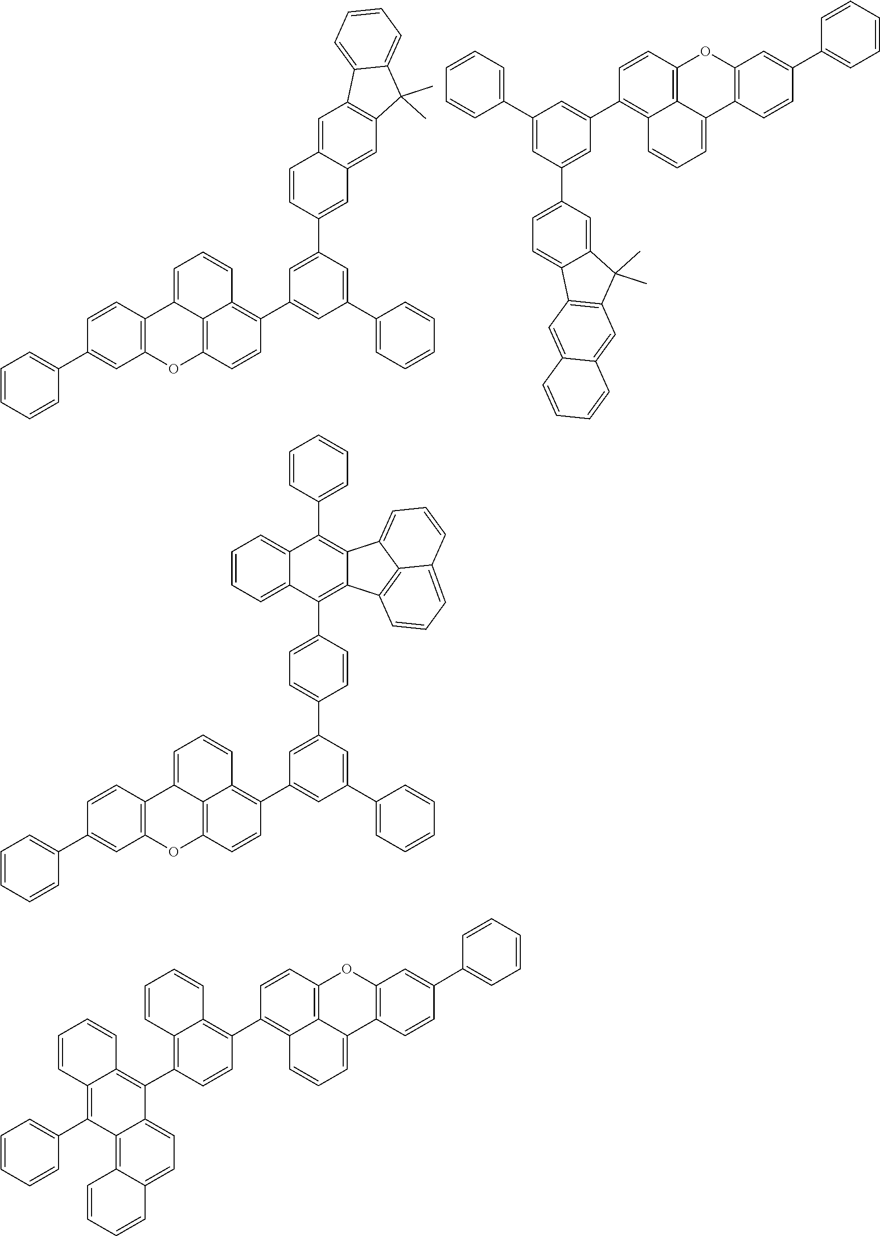

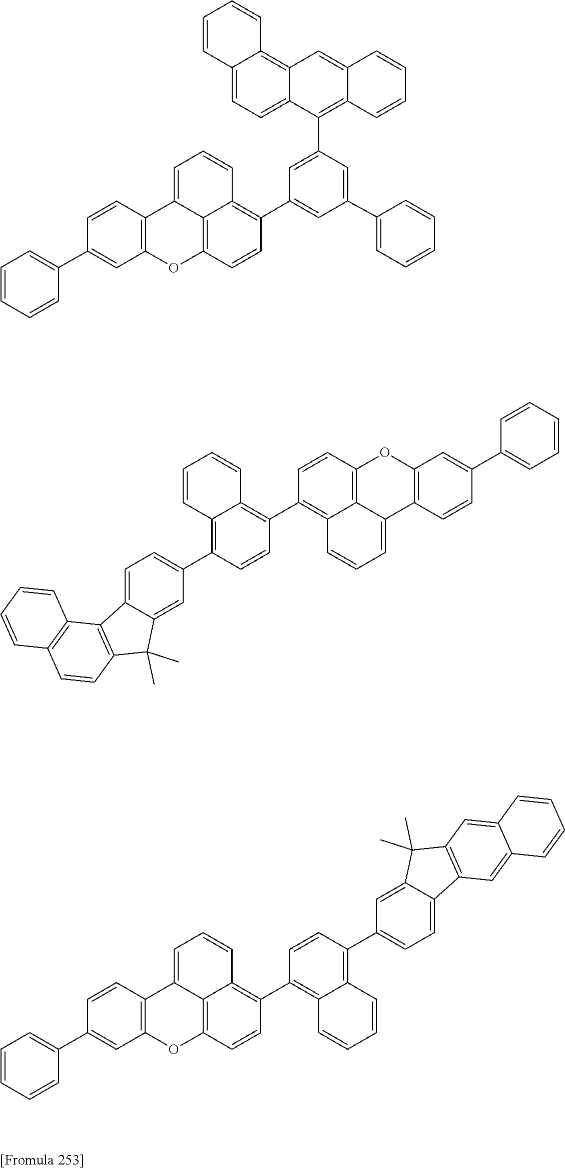 [SMILES: c1ccc(-c2cc(-c3ccc4c5c(cccc35)-c3ccc(-c5ccccc5)cc3O4)cc(-c3c4ccccc4cc4c3ccc3ccccc34)c2)cc1 ; CC1(C)c2cc(-c3ccc(-c4ccc5c6c(cccc46)-c4ccc(-c6ccccc6)cc4O5)c4ccccc34)ccc2-c2c1ccc1ccccc21 ; CC1(C)c2cc(-c3ccc(-c4ccc5c6c(cccc46)-c4ccc(-c6ccccc6)cc4O5)c4ccccc34)ccc2-c2cc3ccccc3cc21]

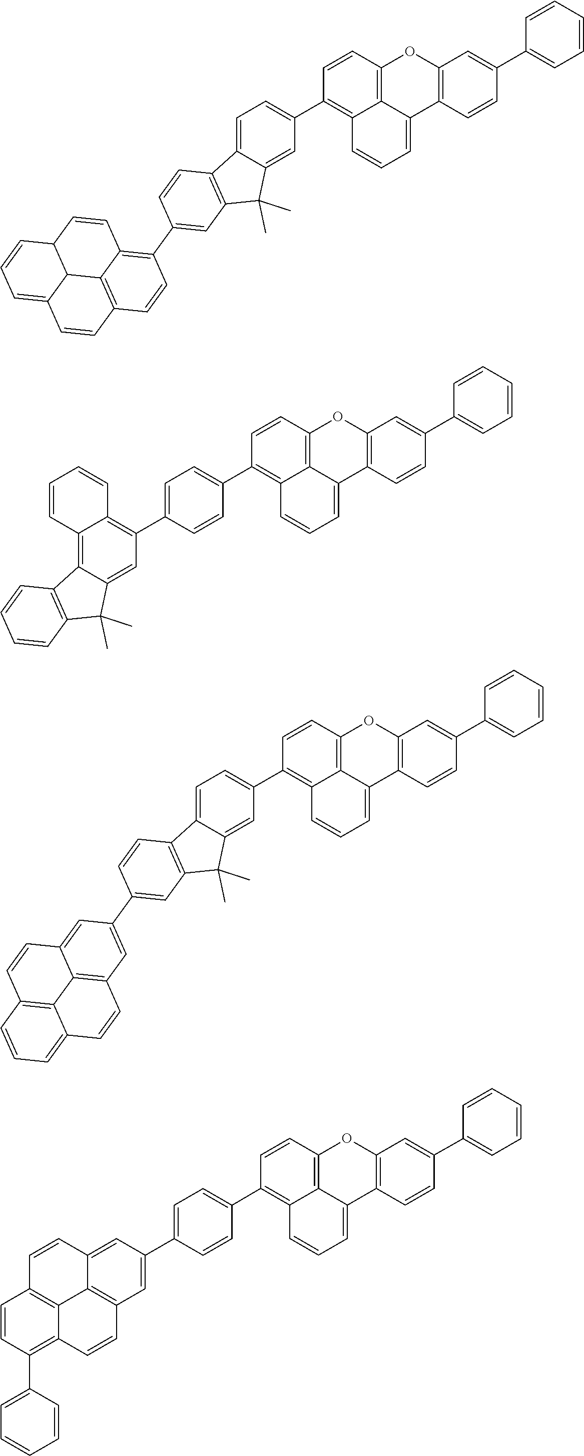 [SMILES: CC1(C)c2cc(-c3ccc4c5c3C=CC3C=CC=C(C=C4)C53)ccc2-c2ccc(-c3ccc4c5c(cccc35)-c3ccc(-c5ccccc5)cc3O4)cc21 ; CC1(C)c2ccccc2-c2c1cc(-c1ccc(-c3ccc4c5c(cccc35)-c3ccc(-c5ccccc5)cc3O4)cc1)c1ccccc21 ; CC1(C)c2cc(-c3cc4ccc5cccc6ccc(c3)c4c56)ccc2-c2ccc(-c3ccc4c5c(cccc35)-c3ccc(-c5ccccc5)cc3O4)cc21 ; c1ccc(-c2ccc3c(c2)Oc2ccc(-c4ccc(-c5cc6ccc7ccc(-c8ccccc8)c8ccc(c5)c6c78)cc4)c4cccc-3c24)cc1]

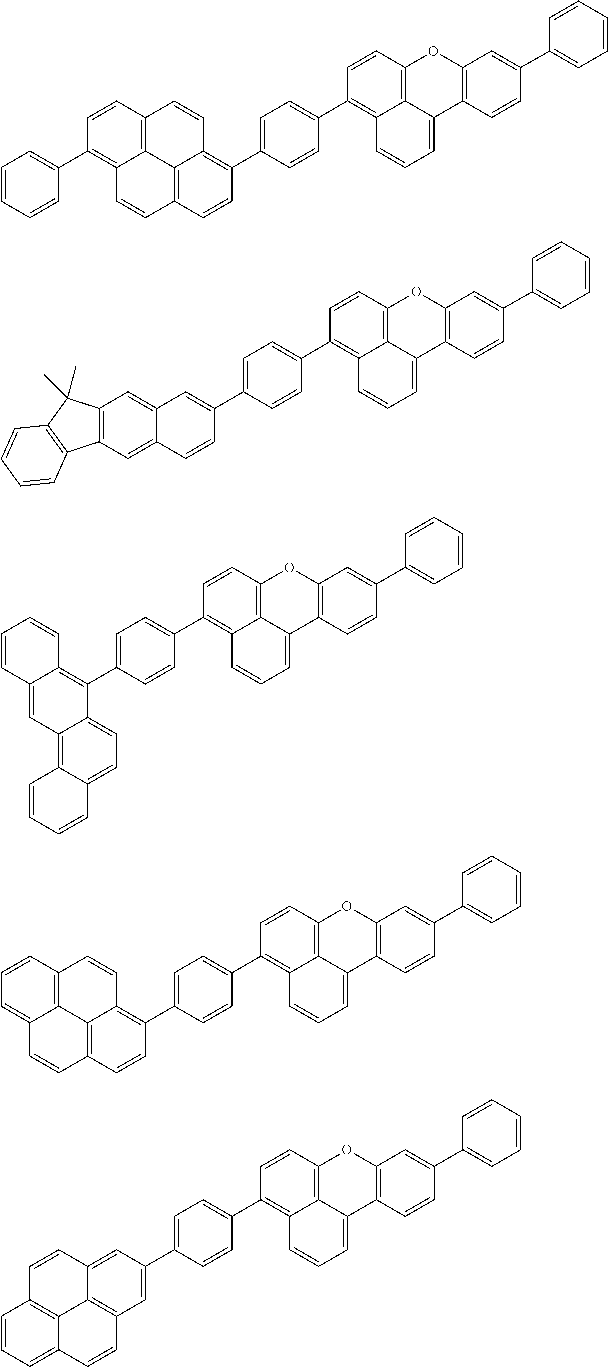 [SMILES: c1ccc(-c2ccc3c(c2)Oc2ccc(-c4ccc(-c5ccc6ccc7c(-c8ccccc8)ccc8ccc5c6c87)cc4)c4cccc-3c24)cc1 ; CC1(C)c2ccccc2-c2cc3ccc(-c4ccc(-c5ccc6c7c(cccc57)-c5ccc(-c7ccccc7)cc5O6)cc4)cc3cc21 ; c1ccc(-c2ccc3c(c2)Oc2ccc(-c4ccc(-c5c6ccccc6cc6c5ccc5ccccc56)cc4)c4cccc-3c24)cc1 ; c1ccc(-c2ccc3c(c2)Oc2ccc(-c4ccc(-c5ccc6ccc7cccc8ccc5c6c78)cc4)c4cccc-3c24)cc1 ; c1ccc(-c2ccc3c(c2)Oc2ccc(-c4ccc(-c5cc6ccc7cccc8ccc(c5)c6c78)cc4)c4cccc-3c24)cc1]

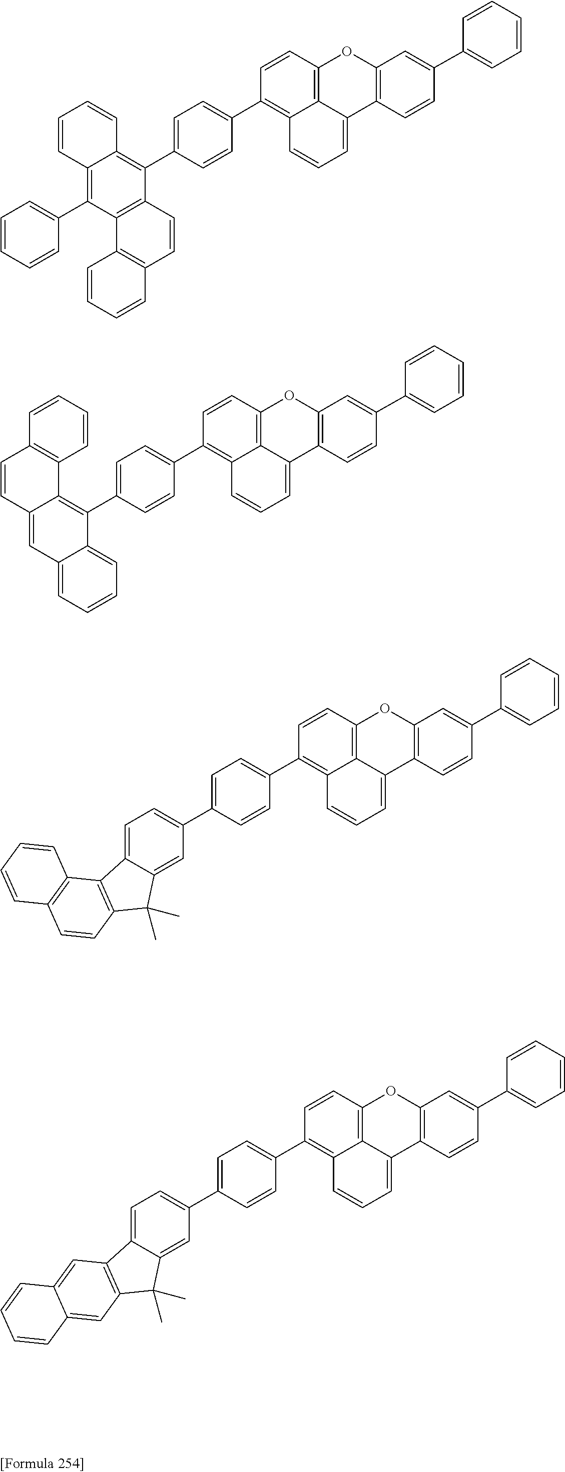 [SMILES: c1ccc(-c2ccc3c(c2)Oc2ccc(-c4ccc(-c5c6ccccc6c(-c6ccccc6)c6c5ccc5ccccc56)cc4)c4cccc-3c24)cc1 ; c1ccc(-c2ccc3c(c2)Oc2ccc(-c4ccc(-c5c6ccccc6cc6ccc7ccccc7c56)cc4)c4cccc-3c24)cc1 ; CC1(C)c2cc(-c3ccc(-c4ccc5c6c(cccc46)-c4ccc(-c6ccccc6)cc4O5)cc3)ccc2-c2c1ccc1ccccc21 ; CC1(C)c2cc(-c3ccc(-c4ccc5c6c(cccc46)-c4ccc(-c6ccccc6)cc4O5)cc3)ccc2-c2cc3ccccc3cc21]

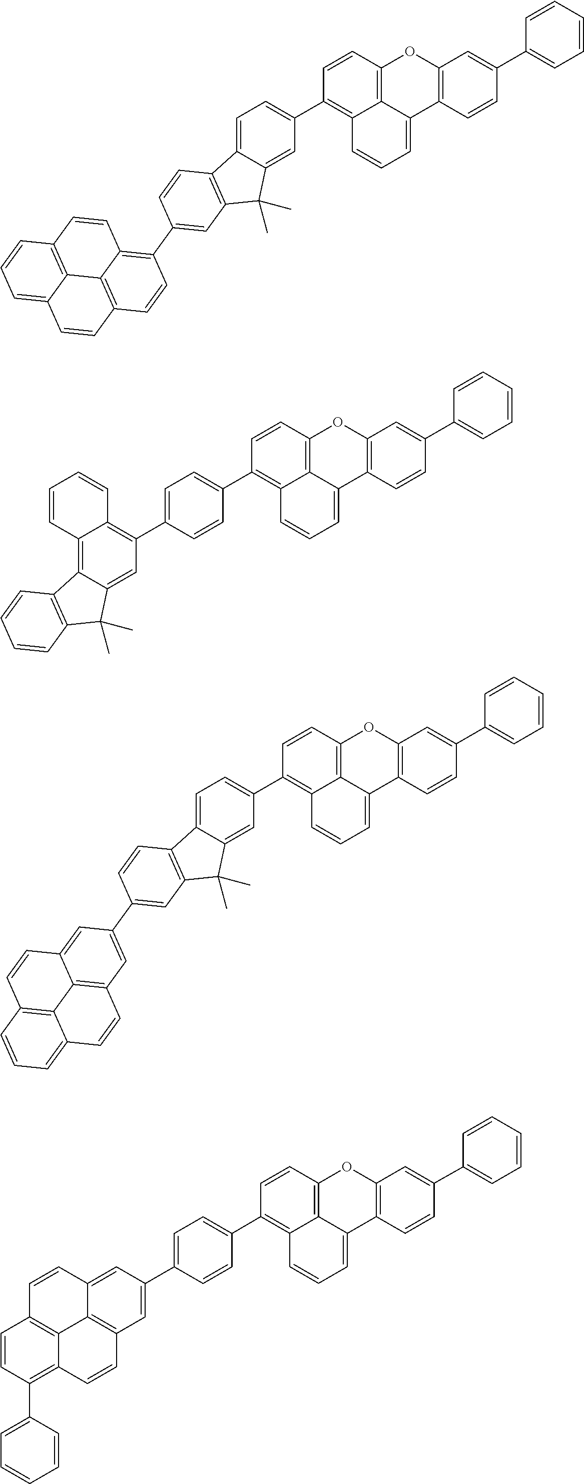 [SMILES: CC1(C)c2cc(-c3ccc4c5c(cccc35)-c3ccc(-c5ccccc5)cc3O4)ccc2-c2ccc(-c3ccc4ccc5cccc6ccc3c4c56)cc21 ; CC1(C)c2ccccc2-c2c1cc(-c1ccc(-c3ccc4c5c(cccc35)-c3ccc(-c5ccccc5)cc3O4)cc1)c1ccccc21 ; CC1(C)c2cc(-c3cc4ccc5cccc6ccc(c3)c4c56)ccc2-c2ccc(-c3ccc4c5c(cccc35)-c3ccc(-c5ccccc5)cc3O4)cc21 ; c1ccc(-c2ccc3c(c2)Oc2ccc(-c4ccc(-c5cc6ccc7ccc(-c8ccccc8)c8ccc(c5)c6c78)cc4)c4cccc-3c24)cc1]

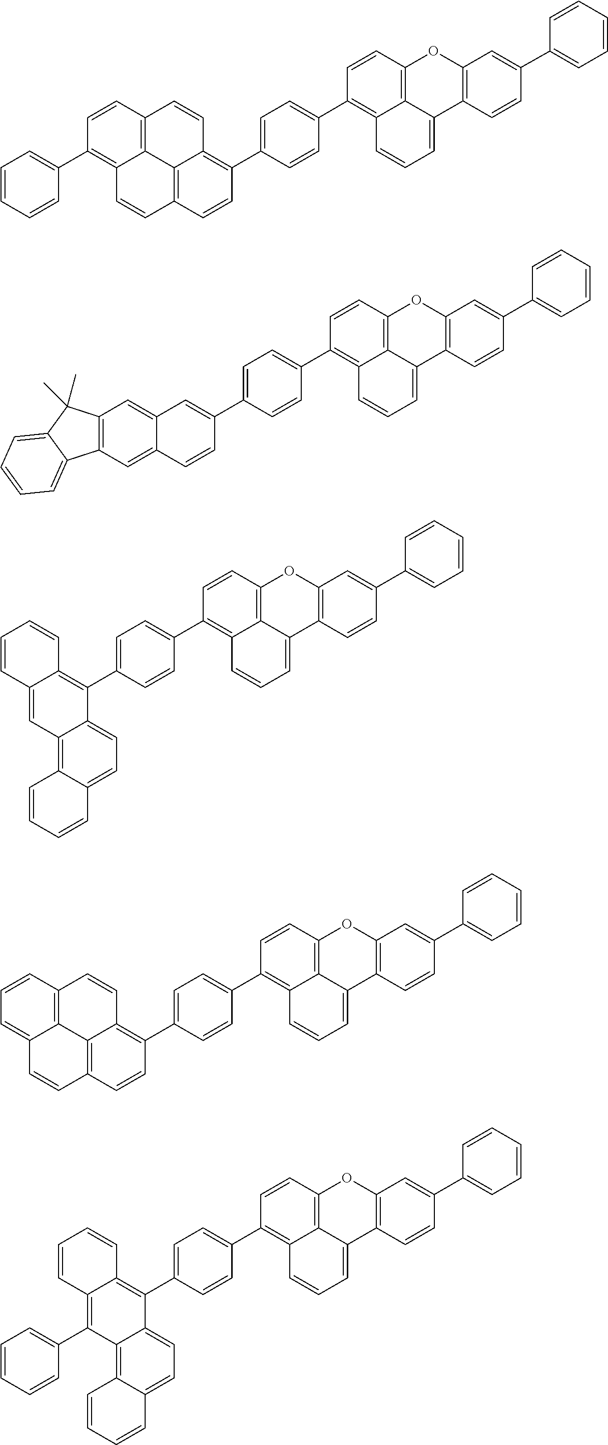 [SMILES: c1ccc(-c2ccc3c(c2)Oc2ccc(-c4ccc(-c5ccc6ccc7c(-c8ccccc8)ccc8ccc5c6c87)cc4)c4cccc-3c24)cc1 ; CC1(C)c2ccccc2-c2cc3ccc(-c4ccc(-c5ccc6c7c(cccc57)-c5ccc(-c7ccccc7)cc5O6)cc4)cc3cc21 ; c1ccc(-c2ccc3c(c2)Oc2ccc(-c4ccc(-c5c6ccccc6cc6c5ccc5ccccc56)cc4)c4cccc-3c24)cc1 ; c1ccc(-c2ccc3c(c2)Oc2ccc(-c4ccc(-c5ccc6ccc7cccc8ccc5c6c78)cc4)c4cccc-3c24)cc1 ; c1ccc(-c2ccc3c(c2)Oc2ccc(-c4ccc(-c5c6ccccc6c(-c6ccccc6)c6c5ccc5ccccc56)cc4)c4cccc-3c24)cc1]

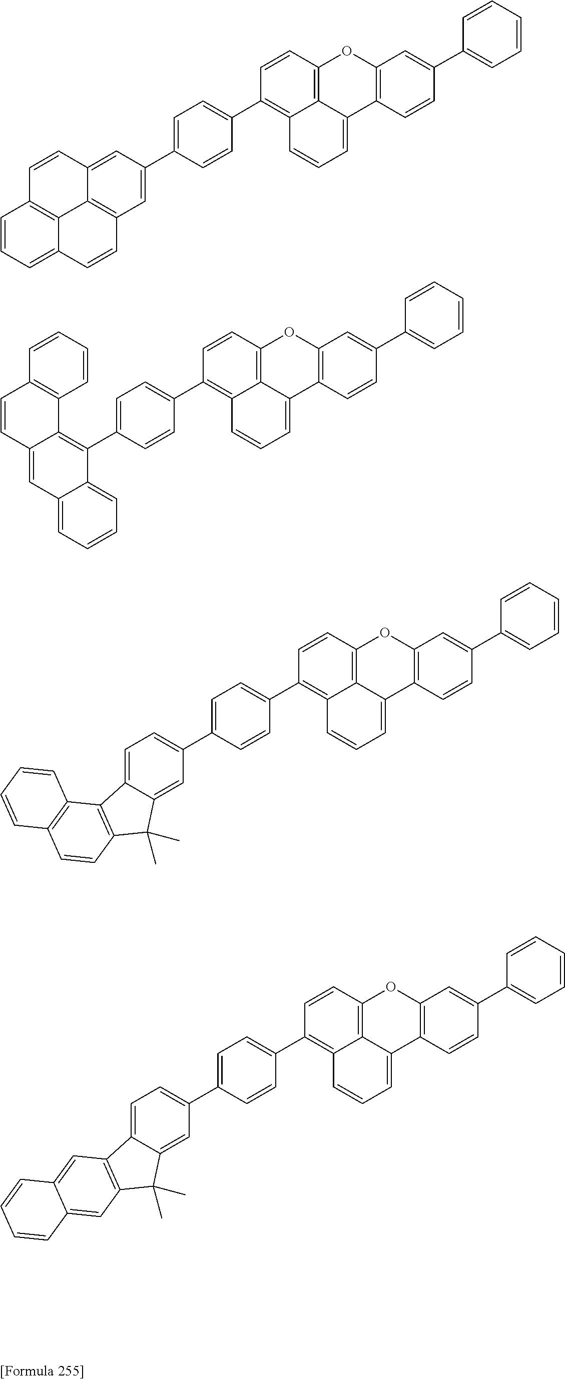 [SMILES: c1ccc(-c2ccc3c(c2)Oc2ccc(-c4ccc(-c5cc6ccc7cccc8ccc(c5)c6c78)cc4)c4cccc-3c24)cc1 ; c1ccc(-c2ccc3c(c2)Oc2ccc(-c4ccc(-c5c6ccccc6cc6ccc7ccccc7c56)cc4)c4cccc-3c24)cc1 ; CC1(C)c2cc(-c3ccc(-c4ccc5c6c(cccc46)-c4ccc(-c6ccccc6)cc4O5)cc3)ccc2-c2c1ccc1ccccc21 ; CC1(C)c2cc(-c3ccc(-c4ccc5c6c(cccc46)-c4ccc(-c6ccccc6)cc4O5)cc3)ccc2-c2cc3ccccc3cc21]

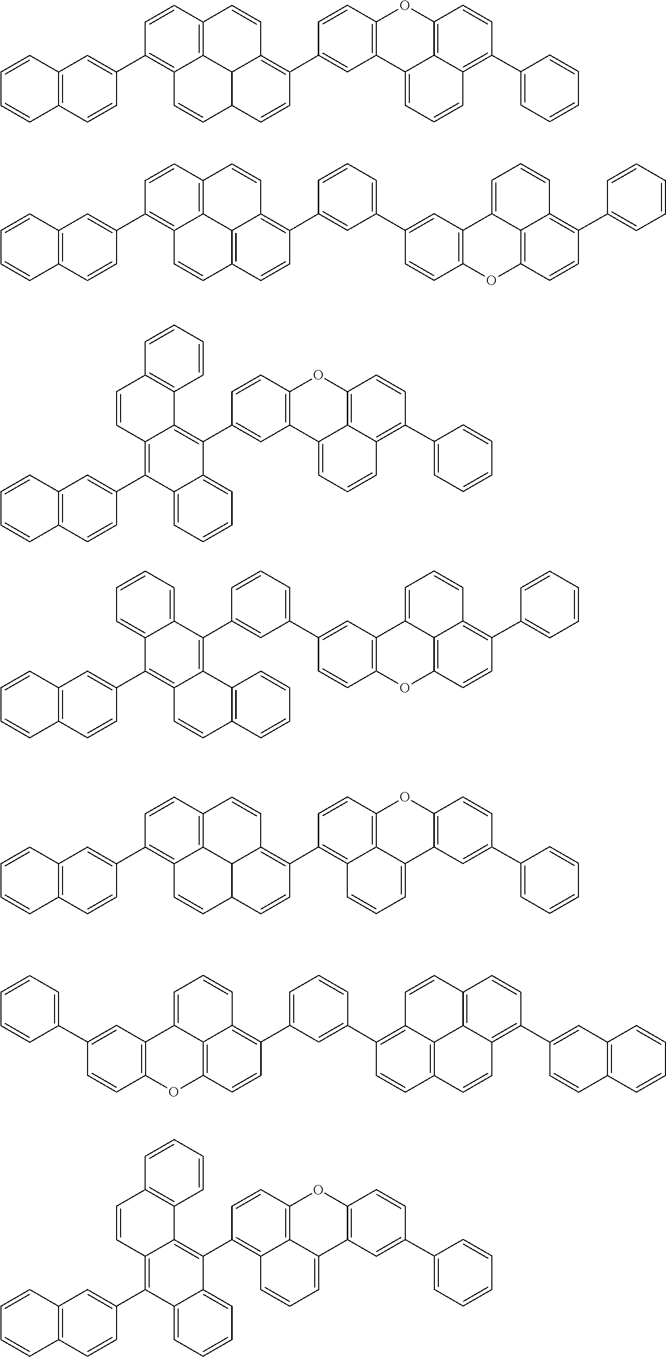

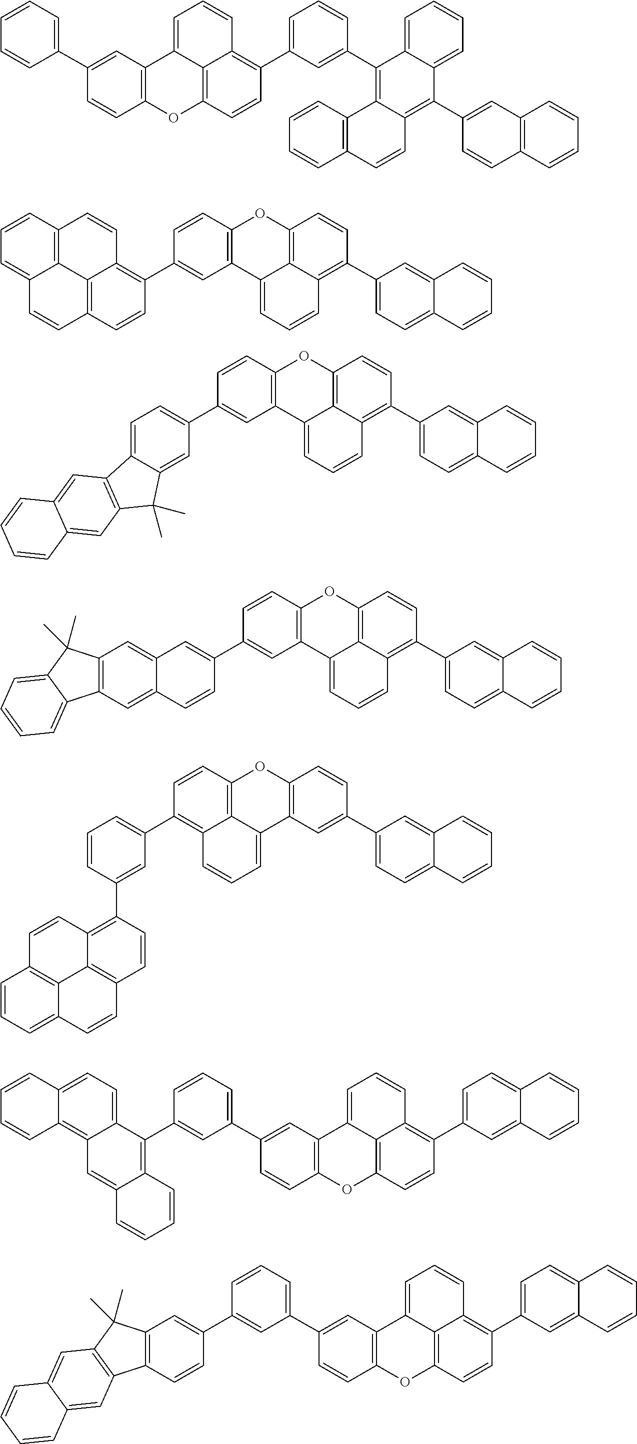

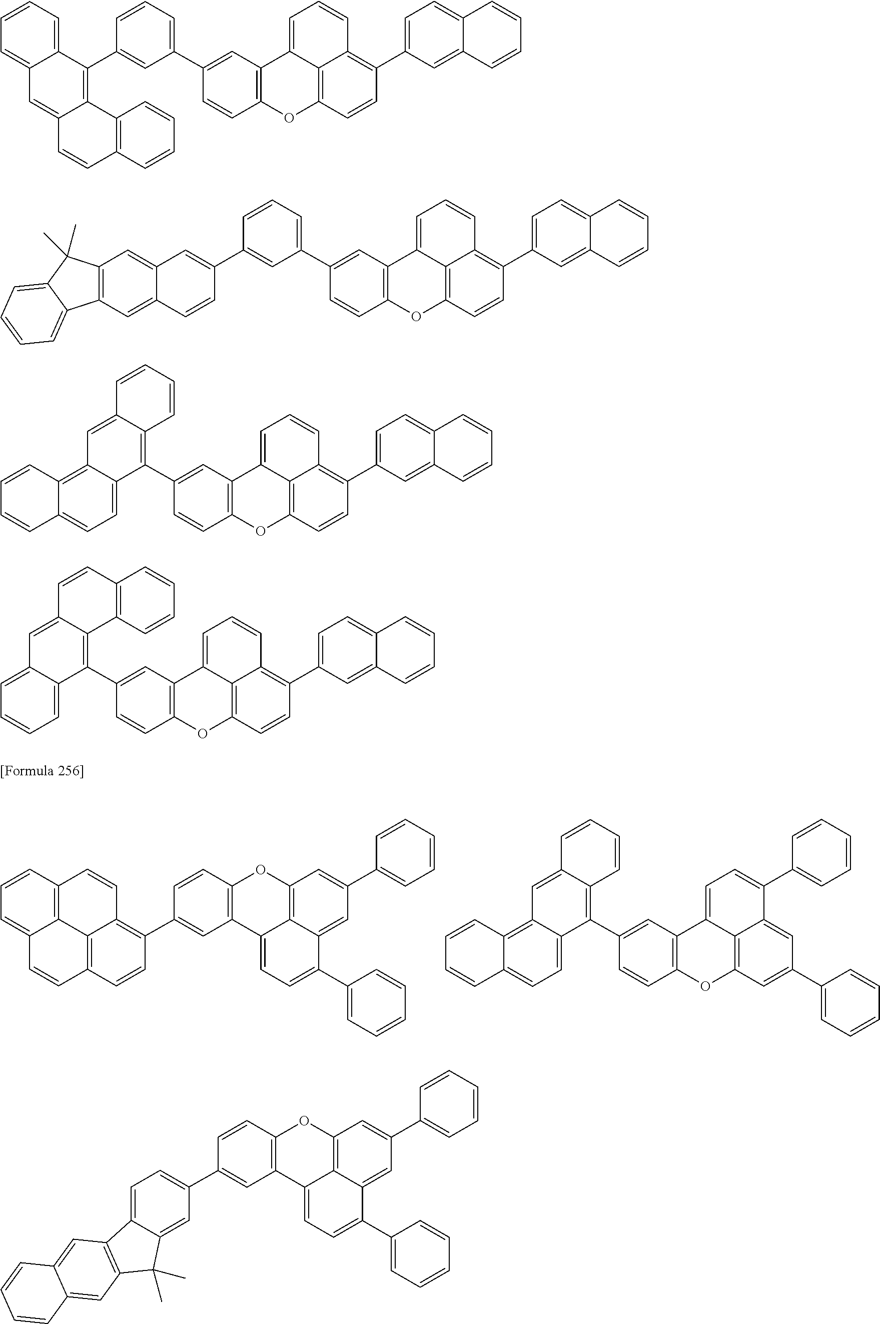

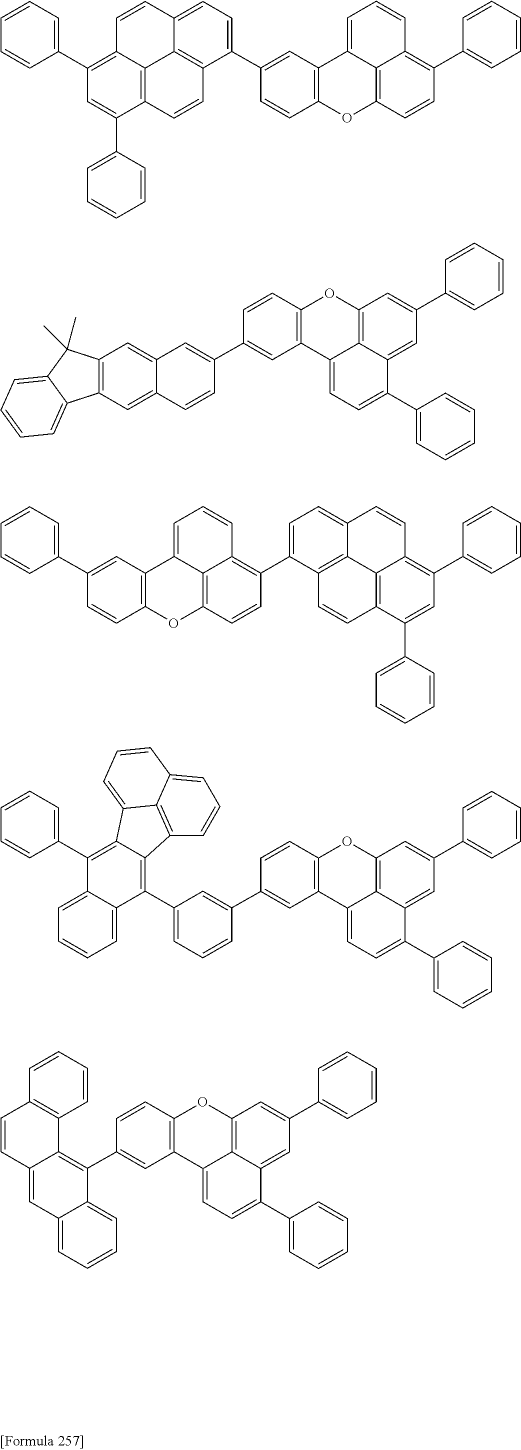 [SMILES: c1ccc(-c2ccc3c4c(cccc24)-c2cc(-c4ccc5ccc6c(-c7ccccc7)cc(-c7ccccc7)c7ccc4c5c67)ccc2O3)cc1 ; CC1(C)c2ccccc2-c2cc3ccc(-c4ccc5c(c4)-c4ccc(-c6ccccc6)c6cc(-c7ccccc7)cc(c46)O5)cc3cc21 ; c1ccc(-c2ccc3c(c2)-c2cccc4c(-c5ccc6ccc7c(-c8ccccc8)cc(-c8ccccc8)c8ccc5c6c78)ccc(c24)O3)cc1 ; c1ccc(-c2cc3c4c(ccc(-c5ccccc5)c4c2)-c2cc(-c4cccc(-c5c6c(c(-c7ccccc7)c7ccccc57)-c5cccc7cccc-6c57)c4)ccc2O3)cc1 ; c1ccc(-c2cc3c4c(ccc(-c5ccccc5)c4c2)-c2cc(-c4c5ccccc5cc5ccc6ccccc6c45)ccc2O3)cc1]

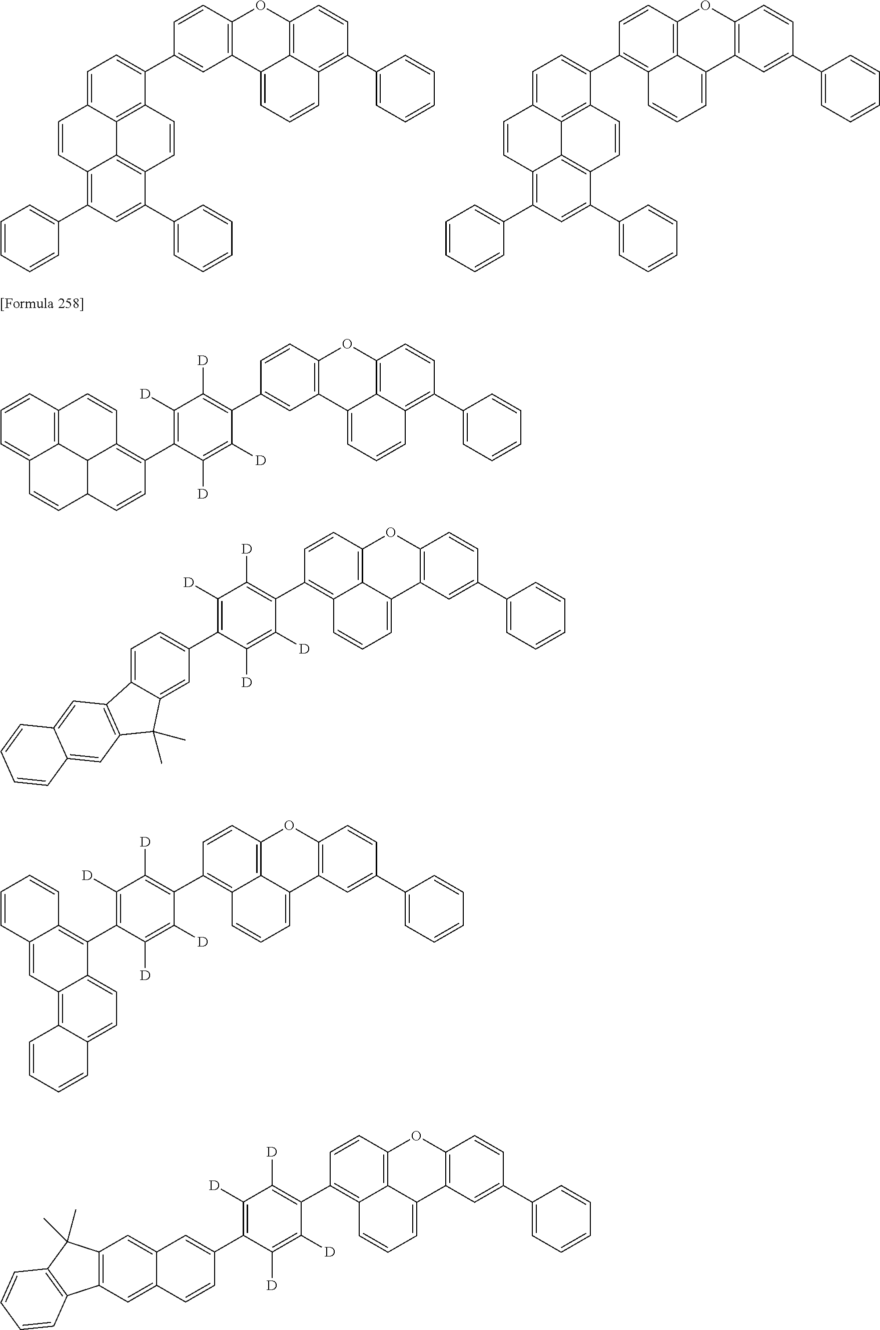

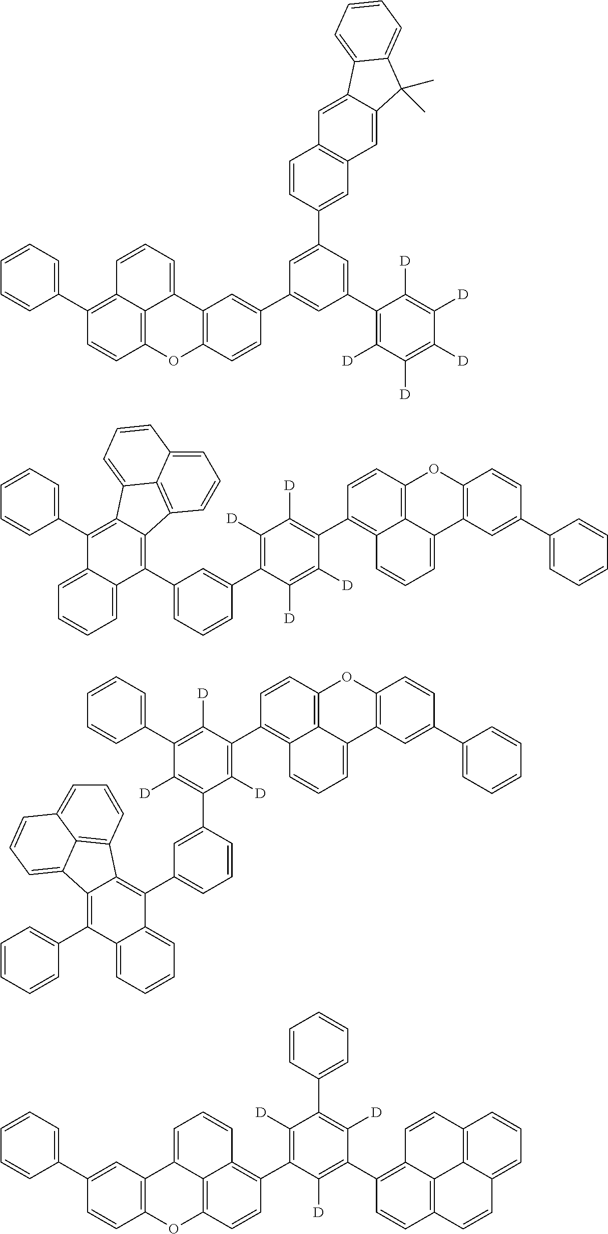 [SMILES: [2H]c1c([2H])c([2H])c(-c2cc(-c3ccc4c(c3)-c3cccc5c(-c6ccccc6)ccc(c35)O4)cc(-c3ccc4cc5c(cc4c3)C(C)(C)c3ccccc3-5)c2)c([2H])c1[2H] ; [2H]c1c([2H])c(-c2ccc3c4c(cccc24)-c2cc(-c4ccccc4)ccc2O3)c([2H])c([2H])c1-c1cccc(-c2c3c(c(-c4ccccc4)c4ccccc24)-c2cccc4cccc-3c24)c1 ; [2H]c1c(-c2ccccc2)c([2H])c(-c2ccc3c4c(cccc24)-c2cc(-c4ccccc4)ccc2O3)c([2H])c1-c1cccc(-c2c3c(c(-c4ccccc4)c4ccccc24)-c2cccc4cccc-3c24)c1 ; [2H]c1c(-c2ccccc2)c([2H])c(-c2ccc3ccc4cccc5ccc2c3c45)c([2H])c1-c1ccc2c3c(cccc13)-c1cc(-c3ccccc3)ccc1O2]

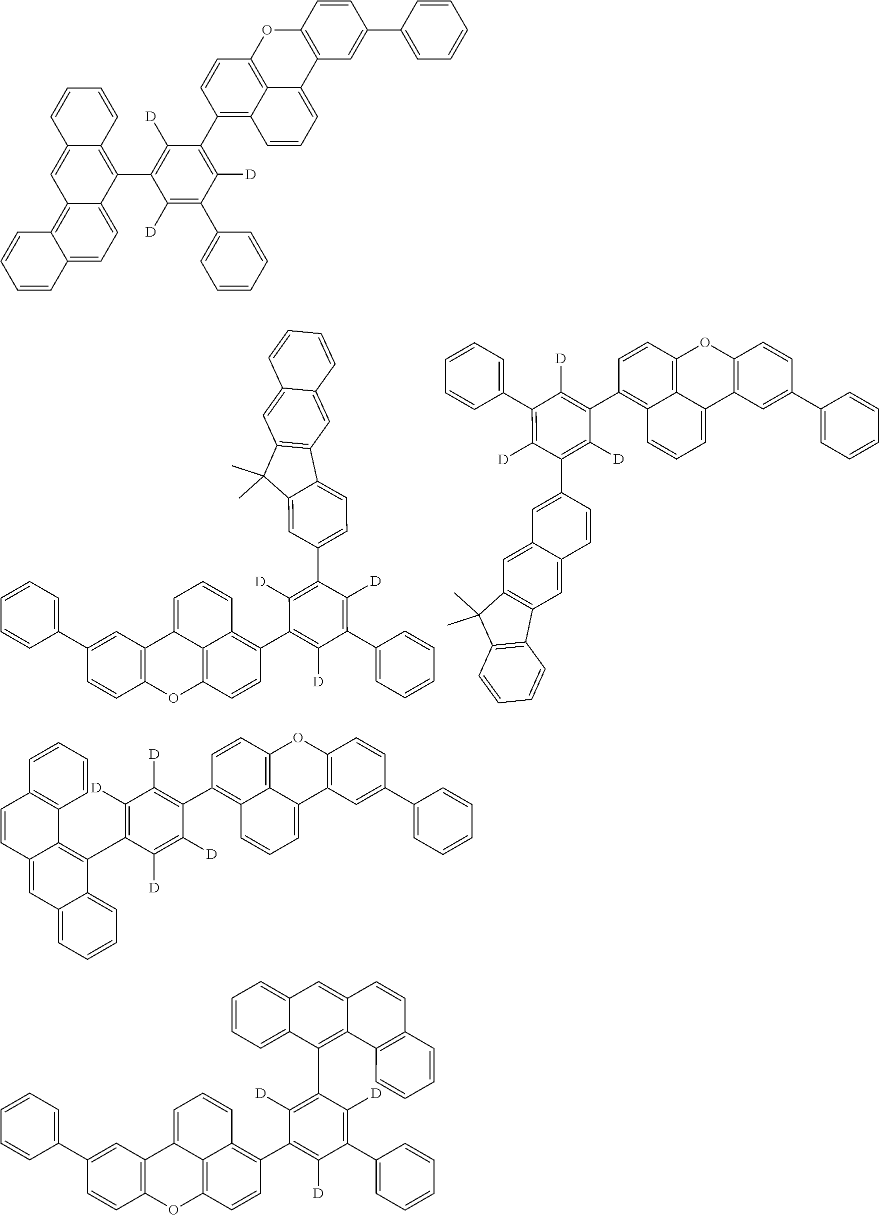

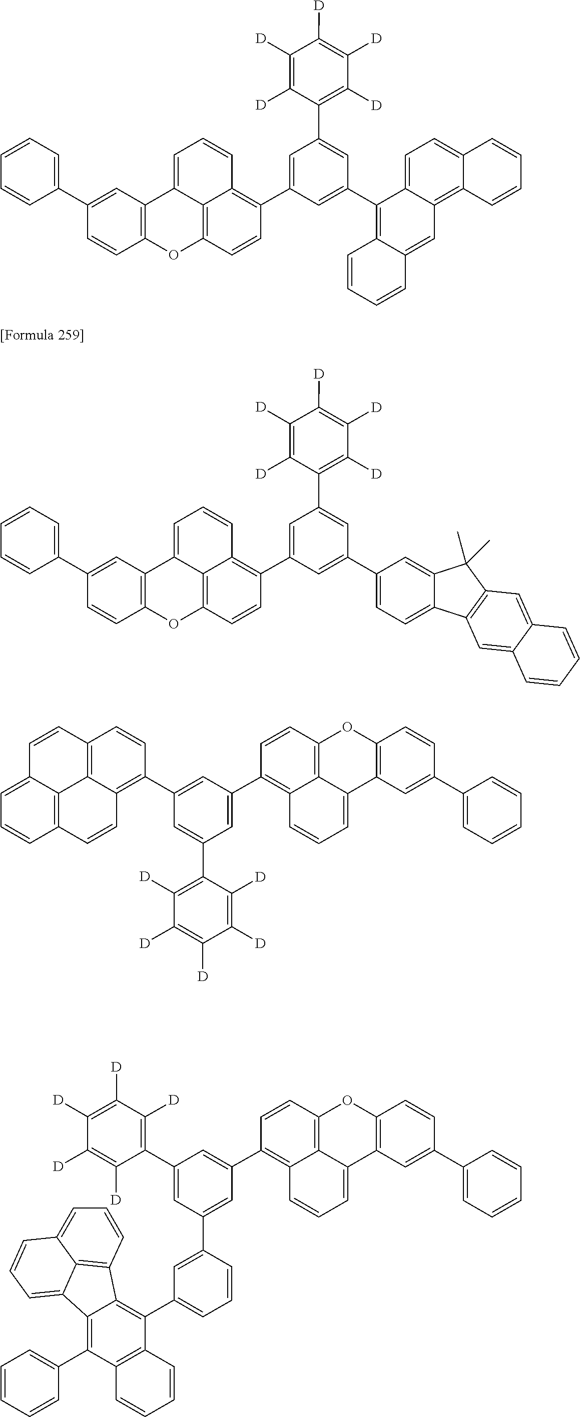

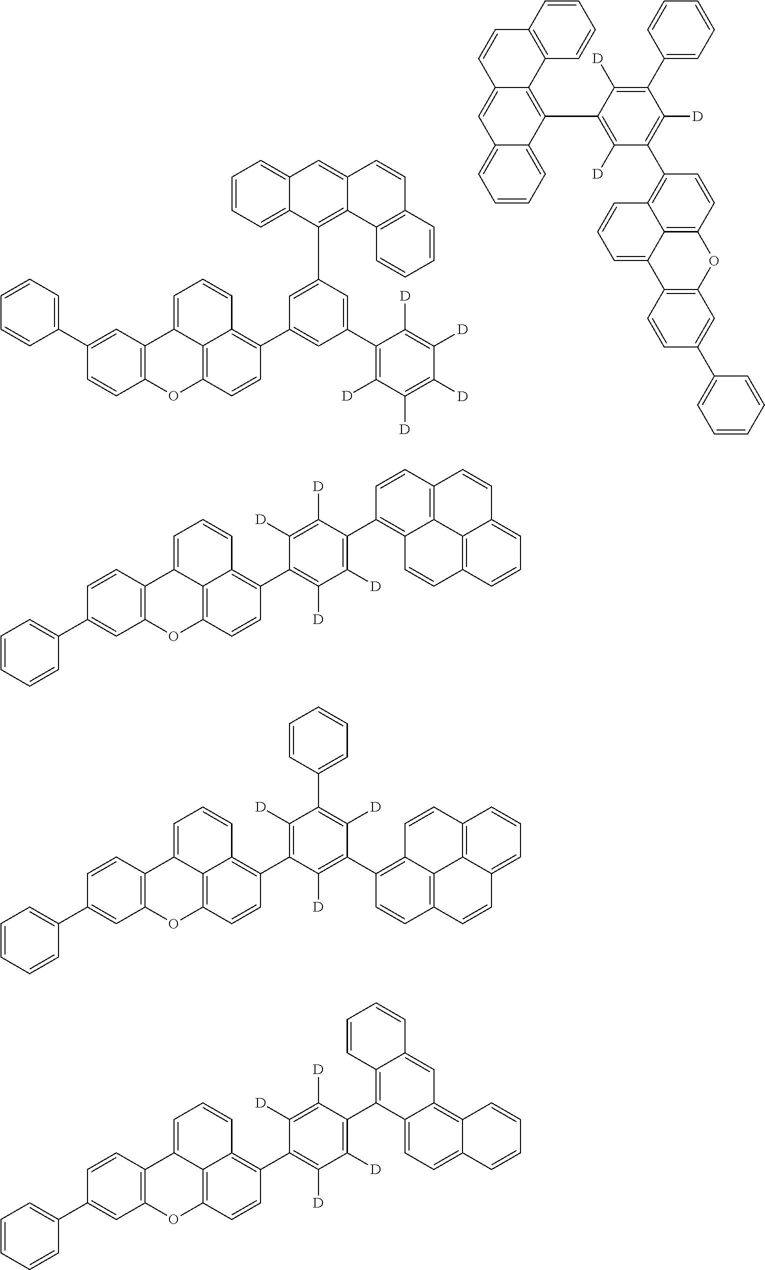

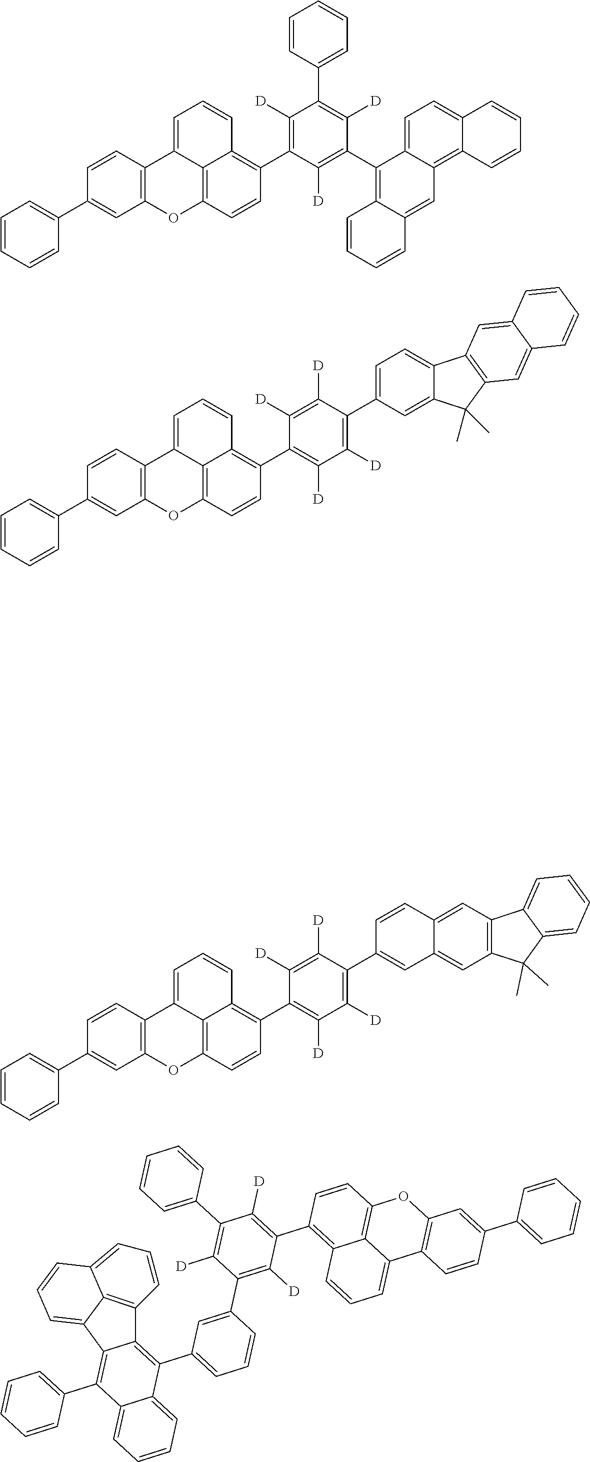 [SMILES: [2H]c1c(-c2ccccc2)c([2H])c(-c2c3ccccc3cc3c2ccc2ccccc23)c([2H])c1-c1ccc2c3c(cccc13)-c1ccc(-c3ccccc3)cc1O2 ; [2H]c1c([2H])c(-c2ccc3c4c(cccc24)-c2ccc(-c4ccccc4)cc2O3)c([2H])c([2H])c1-c1ccc2c(c1)C(C)(C)c1cc3ccccc3cc1-2 ; [2H]c1c([2H])c(-c2ccc3c4c(cccc24)-c2ccc(-c4ccccc4)cc2O3)c([2H])c([2H])c1-c1ccc2cc3c(cc2c1)C(C)(C)c1ccccc1-3 ; [2H]c1c(-c2ccccc2)c([2H])c(-c2ccc3c4c(cccc24)-c2ccc(-c4ccccc4)cc2O3)c([2H])c1-c1cccc(-c2c3c(c(-c4ccccc4)c4ccccc24)-c2cccc4cccc-3c24)c1]

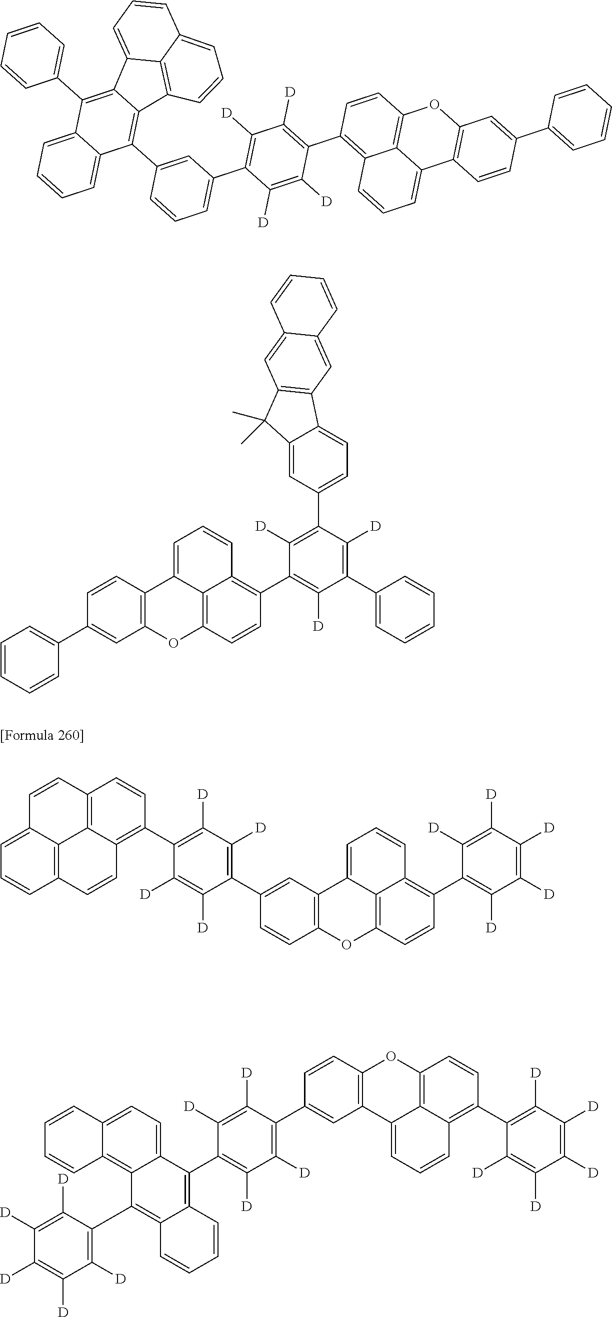

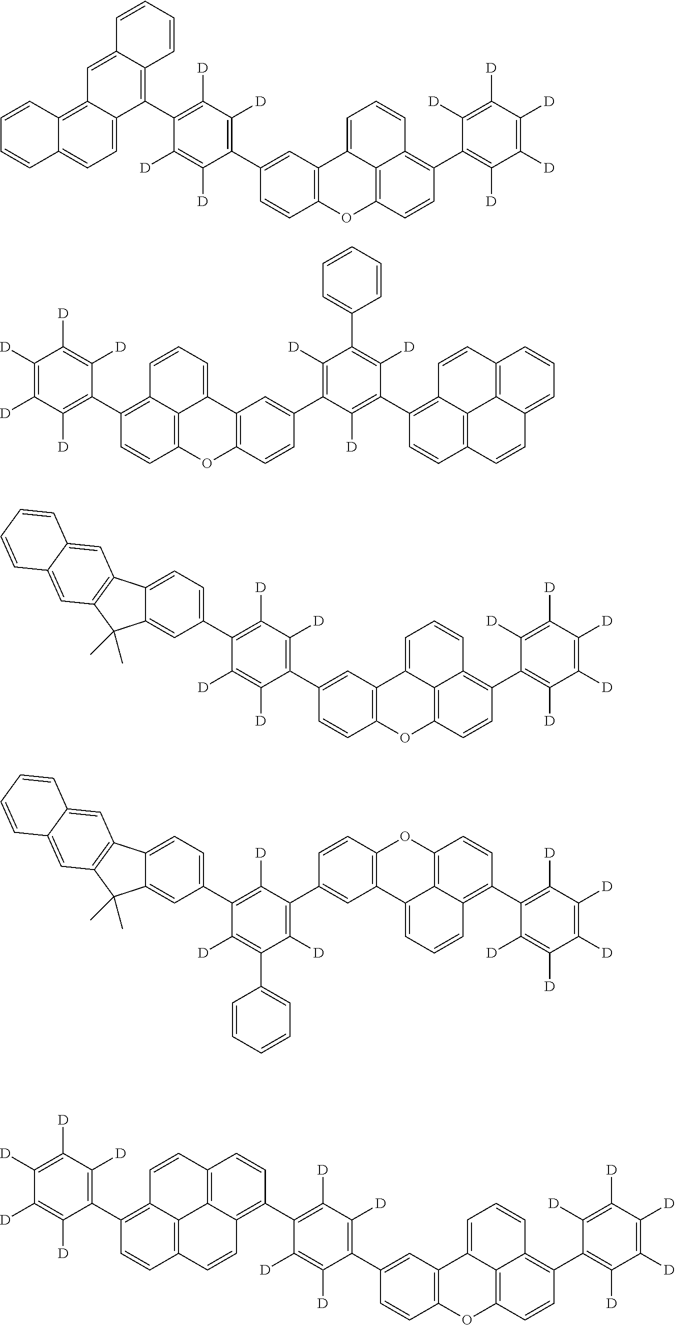

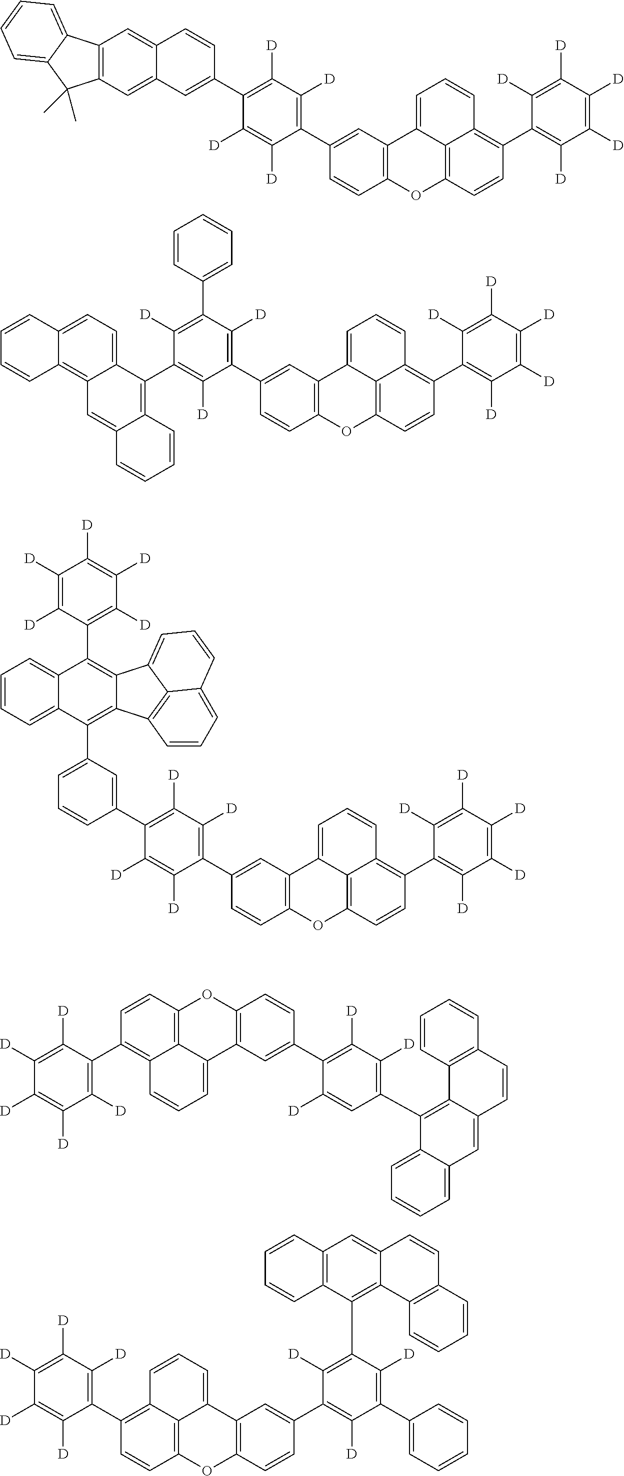

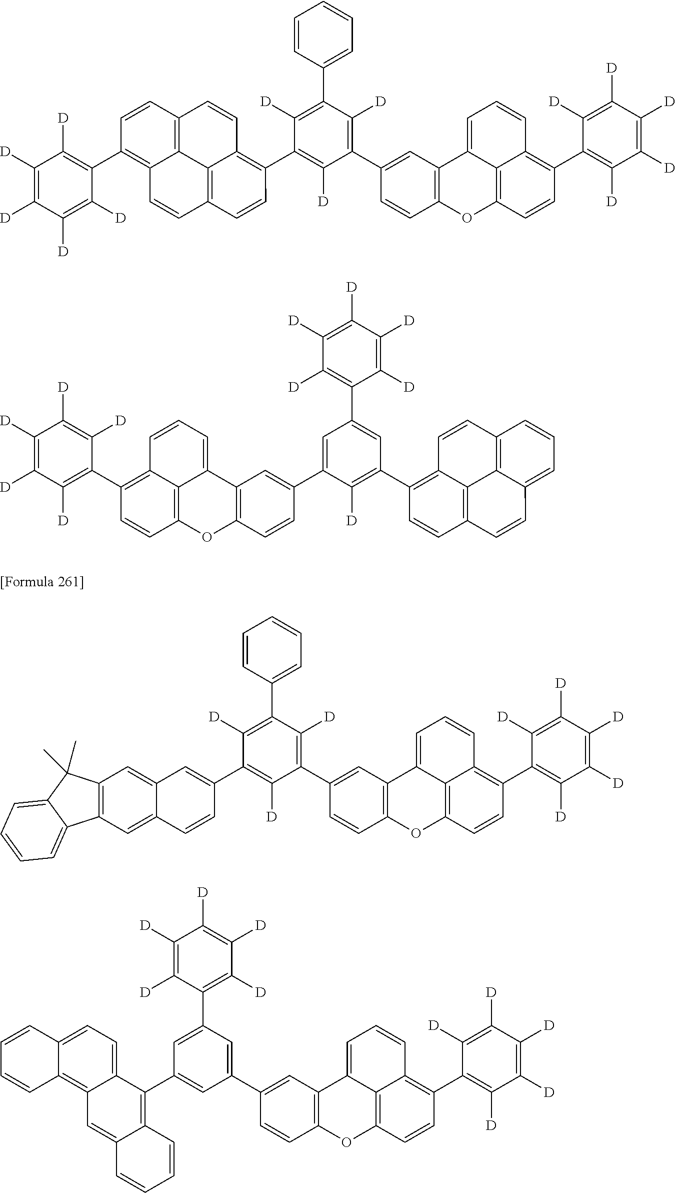

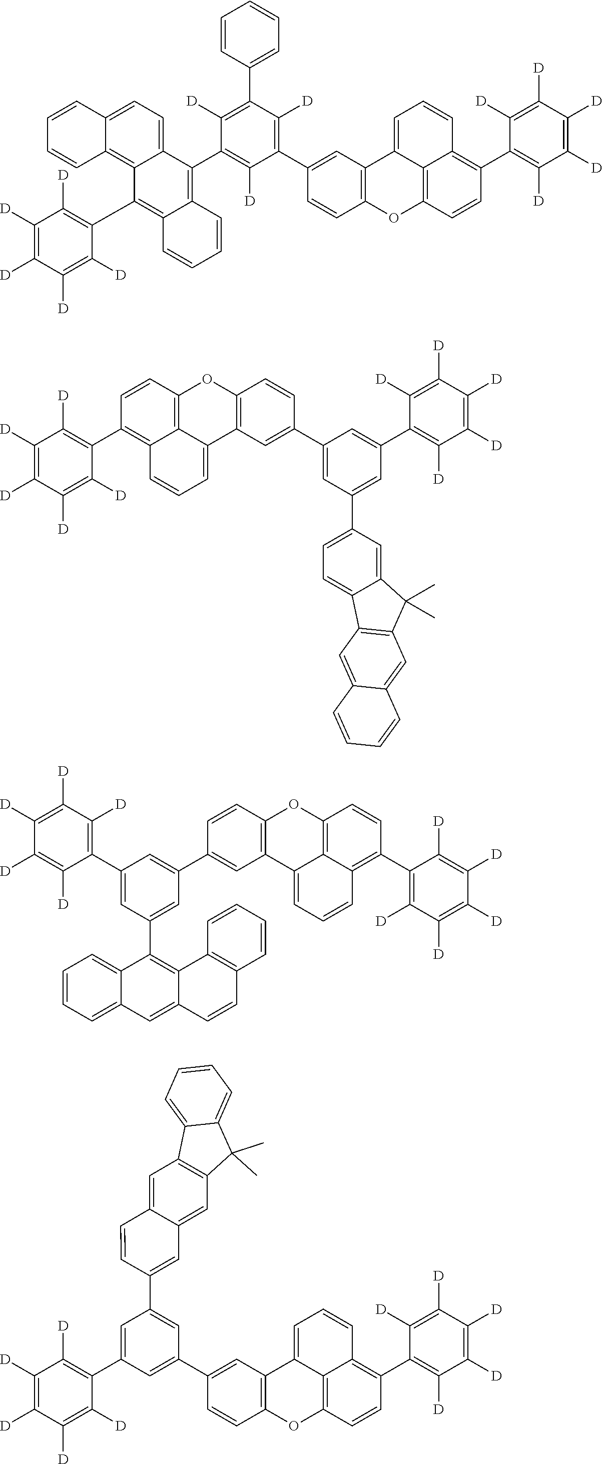

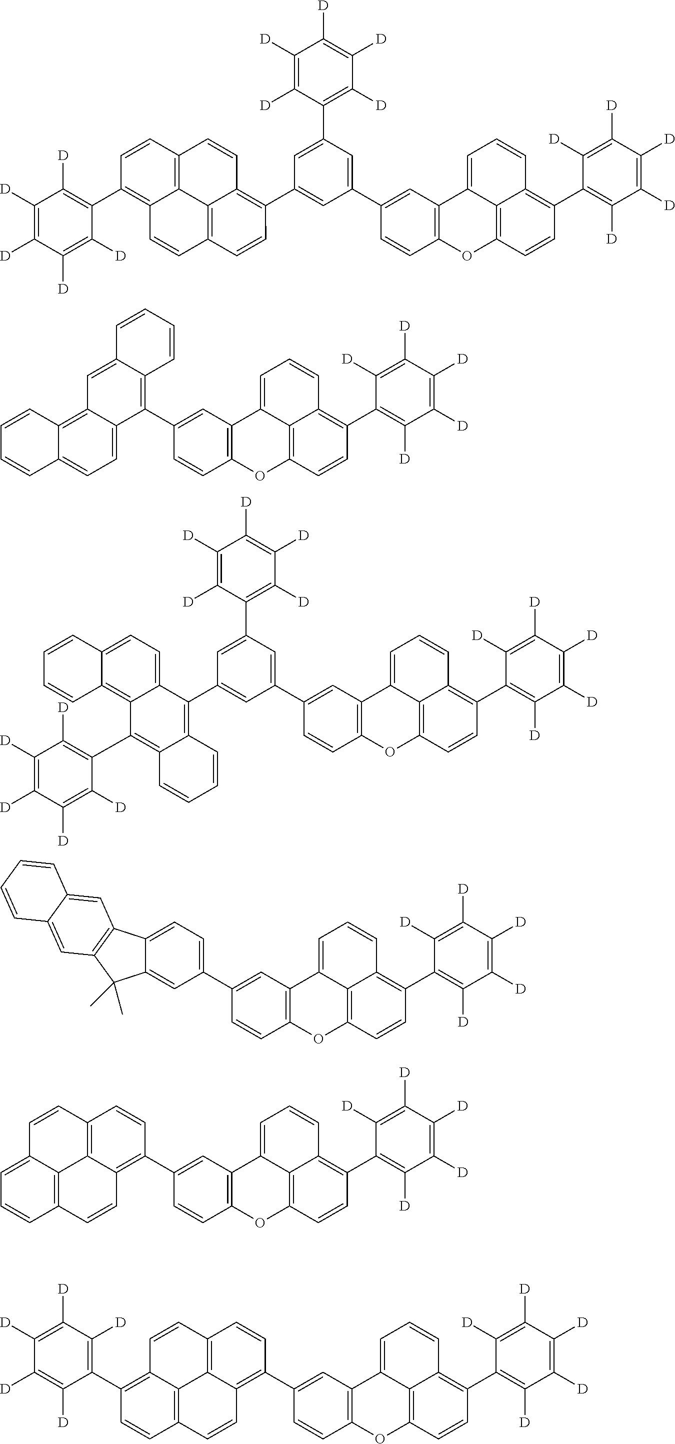

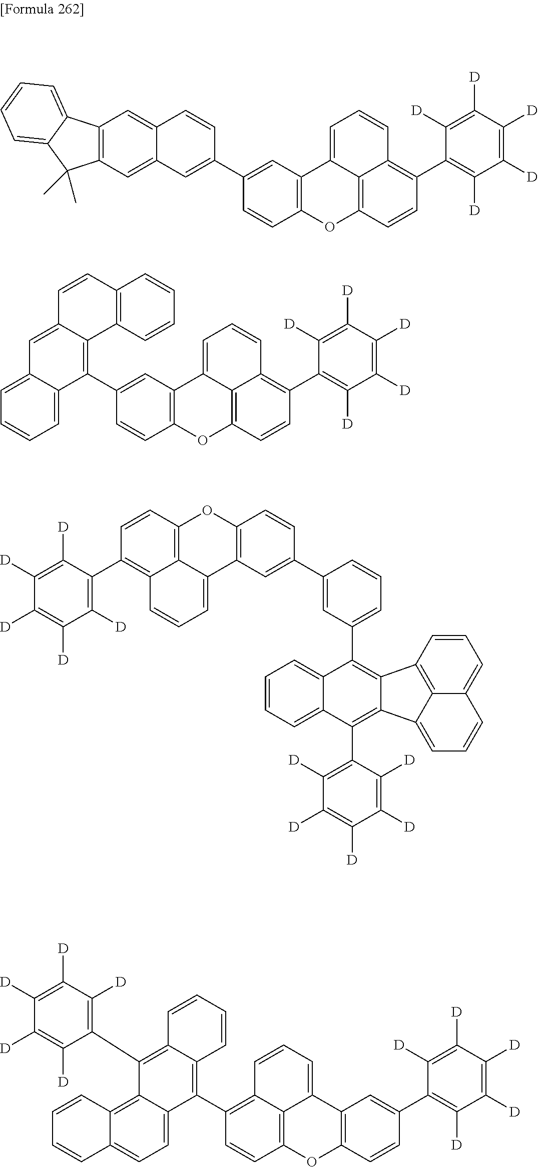 [SMILES: [2H]c1c([2H])c([2H])c(-c2ccc3c4c(cccc24)-c2cc(-c4ccc5cc6c(cc5c4)C(C)(C)c4ccccc4-6)ccc2O3)c([2H])c1[2H] ; [2H]c1c([2H])c([2H])c(-c2ccc3c4c(cccc24)-c2cc(-c4c5ccccc5cc5ccc6ccccc6c45)ccc2O3)c([2H])c1[2H] ; [2H]c1c([2H])c([2H])c(-c2c3c(c(-c4cccc(-c5ccc6c(c5)-c5cccc7c(-c8c([2H])c([2H])c([2H])c([2H])c8[2H])ccc(c57)O6)c4)c4ccccc24)-c2cccc4cccc-3c24)c([2H])c1[2H] ; [2H]c1c([2H])c([2H])c(-c2ccc3c(c2)-c2cccc4c(-c5c6ccccc6c(-c6c([2H])c([2H])c([2H])c([2H])c6[2H])c6c5ccc5ccccc56)ccc(c24)O3)c([2H])c1[2H]]

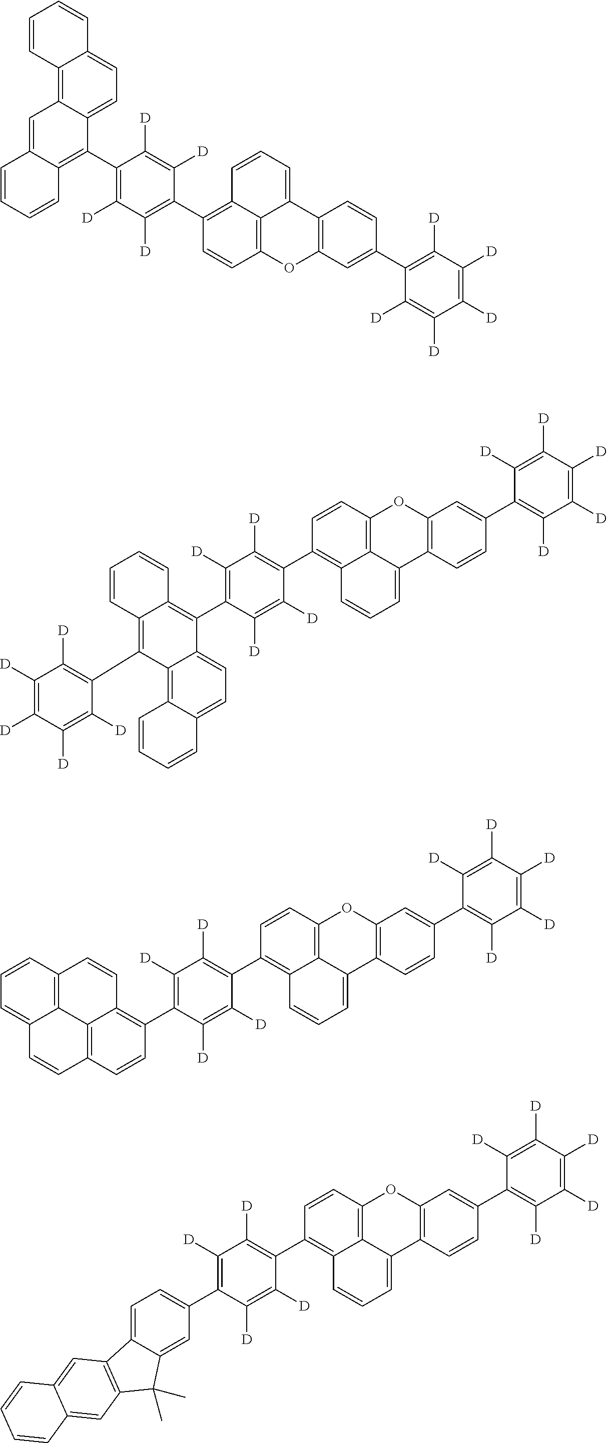

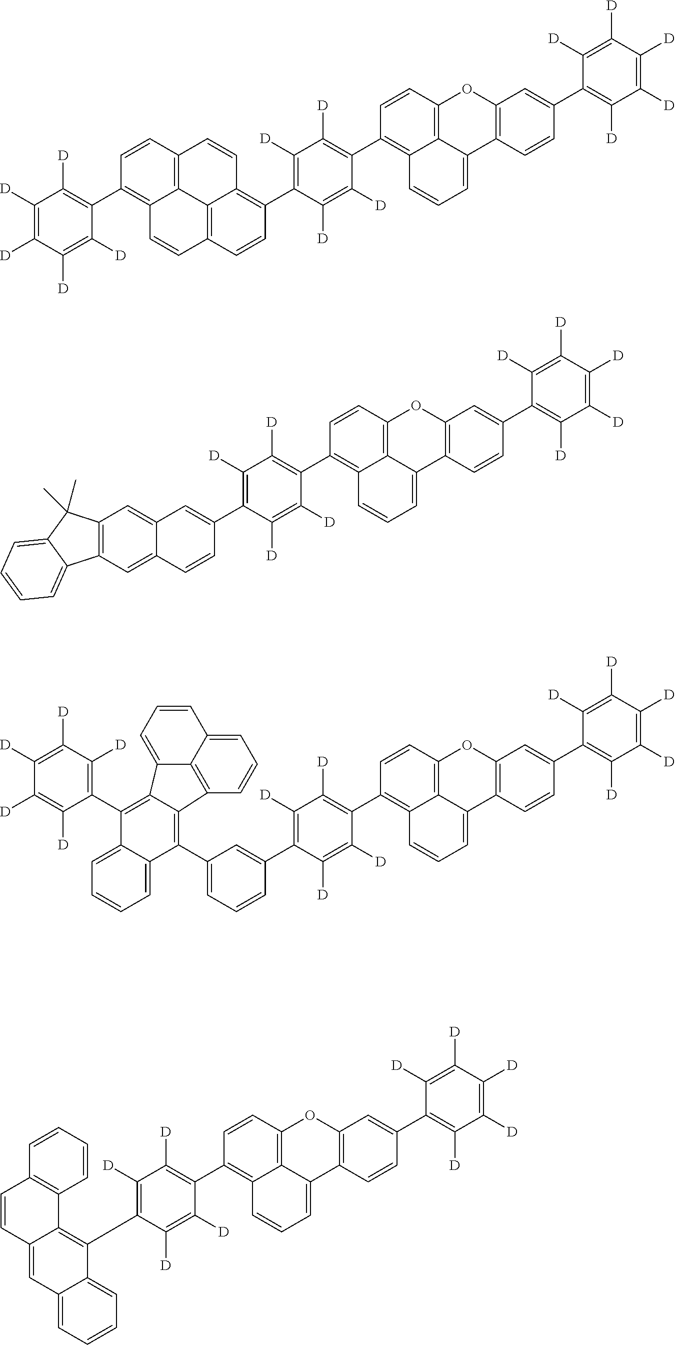

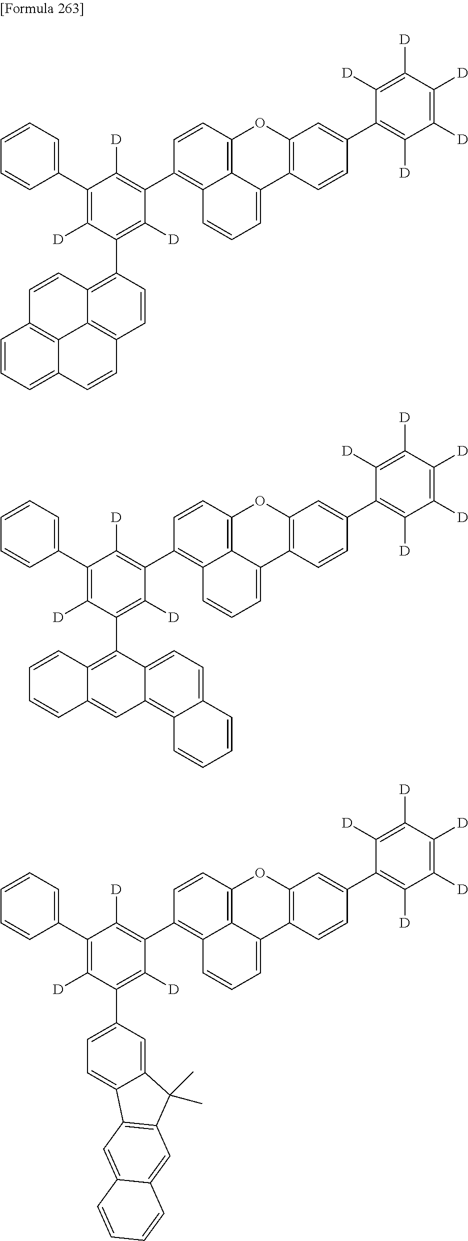 [SMILES: [2H]c1c([2H])c([2H])c(-c2ccc3c(c2)Oc2ccc(-c4c([2H])c(-c5ccccc5)c([2H])c(-c5ccc6ccc7cccc8ccc5c6c78)c4[2H])c4cccc-3c24)c([2H])c1[2H] ; [2H]c1c([2H])c([2H])c(-c2ccc3c(c2)Oc2ccc(-c4c([2H])c(-c5ccccc5)c([2H])c(-c5c6ccccc6cc6c5ccc5ccccc56)c4[2H])c4cccc-3c24)c([2H])c1[2H] ; [2H]c1c([2H])c([2H])c(-c2ccc3c(c2)Oc2ccc(-c4c([2H])c(-c5ccccc5)c([2H])c(-c5ccc6c(c5)C(C)(C)c5cc7ccccc7cc5-6)c4[2H])c4cccc-3c24)c([2H])c1[2H]]

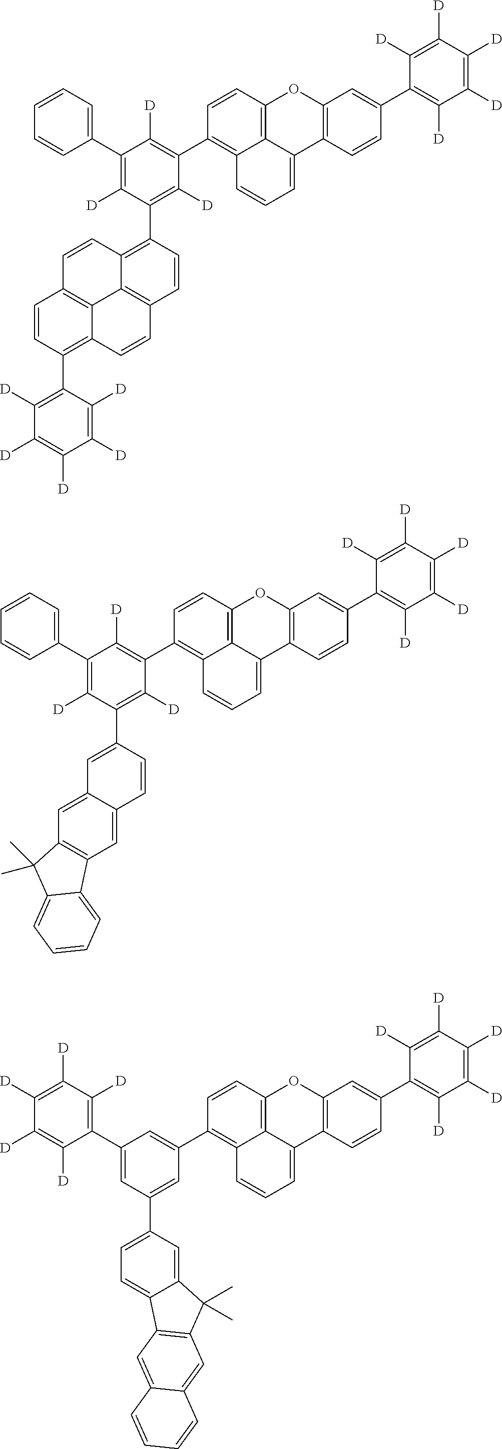 [SMILES: [2H]c1c([2H])c([2H])c(-c2ccc3c(c2)Oc2ccc(-c4c([2H])c(-c5ccccc5)c([2H])c(-c5ccc6ccc7c(-c8c([2H])c([2H])c([2H])c([2H])c8[2H])ccc8ccc5c6c87)c4[2H])c4cccc-3c24)c([2H])c1[2H] ; [2H]c1c([2H])c([2H])c(-c2ccc3c(c2)Oc2ccc(-c4c([2H])c(-c5ccccc5)c([2H])c(-c5ccc6cc7c(cc6c5)C(C)(C)c5ccccc5-7)c4[2H])c4cccc-3c24)c([2H])c1[2H] ; [2H]c1c([2H])c([2H])c(-c2cc(-c3ccc4c(c3)C(C)(C)c3cc5ccccc5cc3-4)cc(-c3ccc4c5c(cccc35)-c3ccc(-c5c([2H])c([2H])c([2H])c([2H])c5[2H])cc3O4)c2)c([2H])c1[2H]]

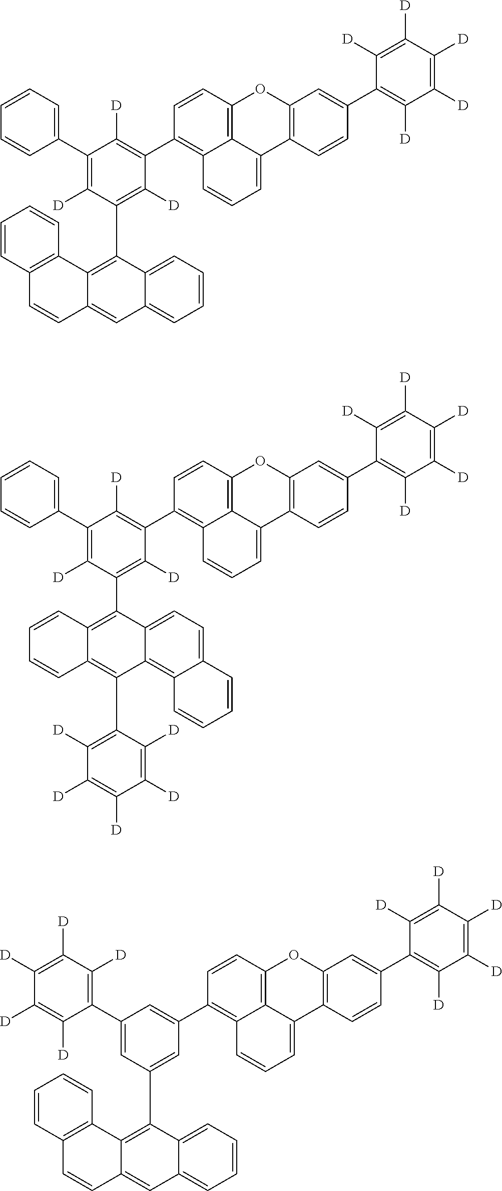 [SMILES: [2H]c1c([2H])c([2H])c(-c2ccc3c(c2)Oc2ccc(-c4c([2H])c(-c5ccccc5)c([2H])c(-c5c6ccccc6cc6ccc7ccccc7c56)c4[2H])c4cccc-3c24)c([2H])c1[2H] ; [2H]c1c([2H])c([2H])c(-c2ccc3c(c2)Oc2ccc(-c4c([2H])c(-c5ccccc5)c([2H])c(-c5c6ccccc6c(-c6c([2H])c([2H])c([2H])c([2H])c6[2H])c6c5ccc5ccccc56)c4[2H])c4cccc-3c24)c([2H])c1[2H] ; [2H]c1c([2H])c([2H])c(-c2cc(-c3ccc4c5c(cccc35)-c3ccc(-c5c([2H])c([2H])c([2H])c([2H])c5[2H])cc3O4)cc(-c3c4ccccc4cc4ccc5ccccc5c34)c2)c([2H])c1[2H]]

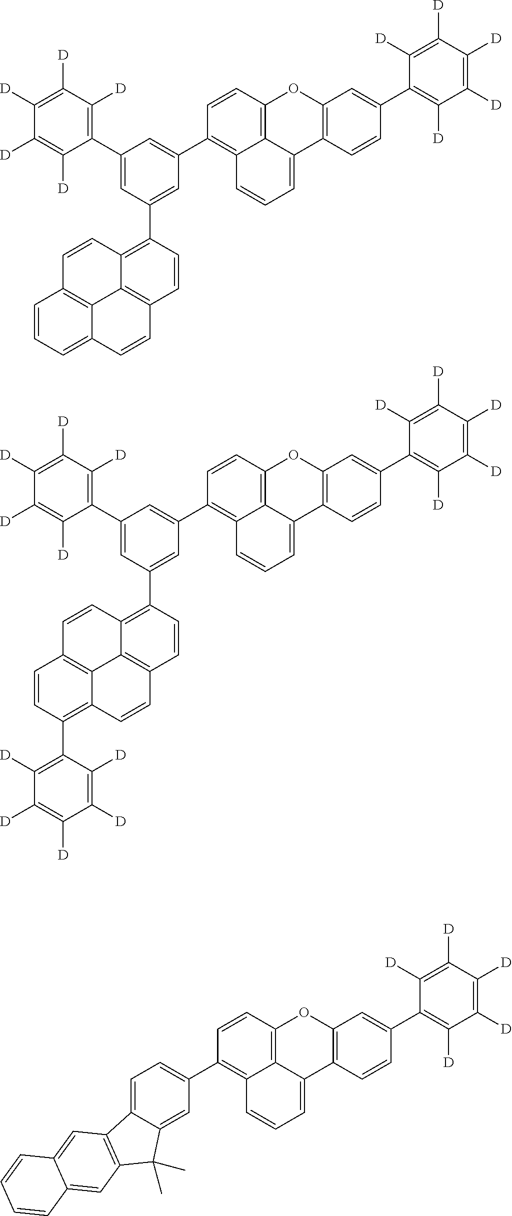 [SMILES: [2H]c1c([2H])c([2H])c(-c2cc(-c3ccc4c5c(cccc35)-c3ccc(-c5c([2H])c([2H])c([2H])c([2H])c5[2H])cc3O4)cc(-c3ccc4ccc5cccc6ccc3c4c56)c2)c([2H])c1[2H] ; [2H]c1c([2H])c([2H])c(-c2cc(-c3ccc4c5c(cccc35)-c3ccc(-c5c([2H])c([2H])c([2H])c([2H])c5[2H])cc3O4)cc(-c3ccc4ccc5c(-c6c([2H])c([2H])c([2H])c([2H])c6[2H])ccc6ccc3c4c65)c2)c([2H])c1[2H] ; [2H]c1c([2H])c([2H])c(-c2ccc3c(c2)Oc2ccc(-c4ccc5c(c4)C(C)(C)c4cc6ccccc6cc4-5)c4cccc-3c24)c([2H])c1[2H]]

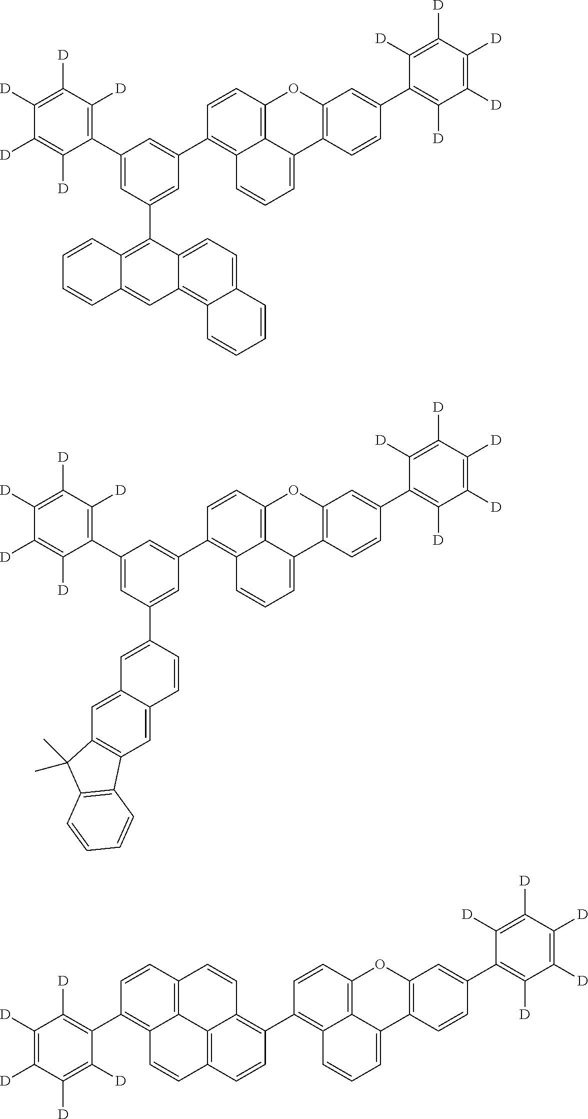 [SMILES: [2H]c1c([2H])c([2H])c(-c2cc(-c3ccc4c5c(cccc35)-c3ccc(-c5c([2H])c([2H])c([2H])c([2H])c5[2H])cc3O4)cc(-c3c4ccccc4cc4c3ccc3ccccc34)c2)c([2H])c1[2H] ; [2H]c1c([2H])c([2H])c(-c2cc(-c3ccc4cc5c(cc4c3)C(C)(C)c3ccccc3-5)cc(-c3ccc4c5c(cccc35)-c3ccc(-c5c([2H])c([2H])c([2H])c([2H])c5[2H])cc3O4)c2)c([2H])c1[2H] ; [2H]c1c([2H])c([2H])c(-c2ccc3c(c2)Oc2ccc(-c4ccc5ccc6c(-c7c([2H])c([2H])c([2H])c([2H])c7[2H])ccc7ccc4c5c76)c4cccc-3c24)c([2H])c1[2H]]

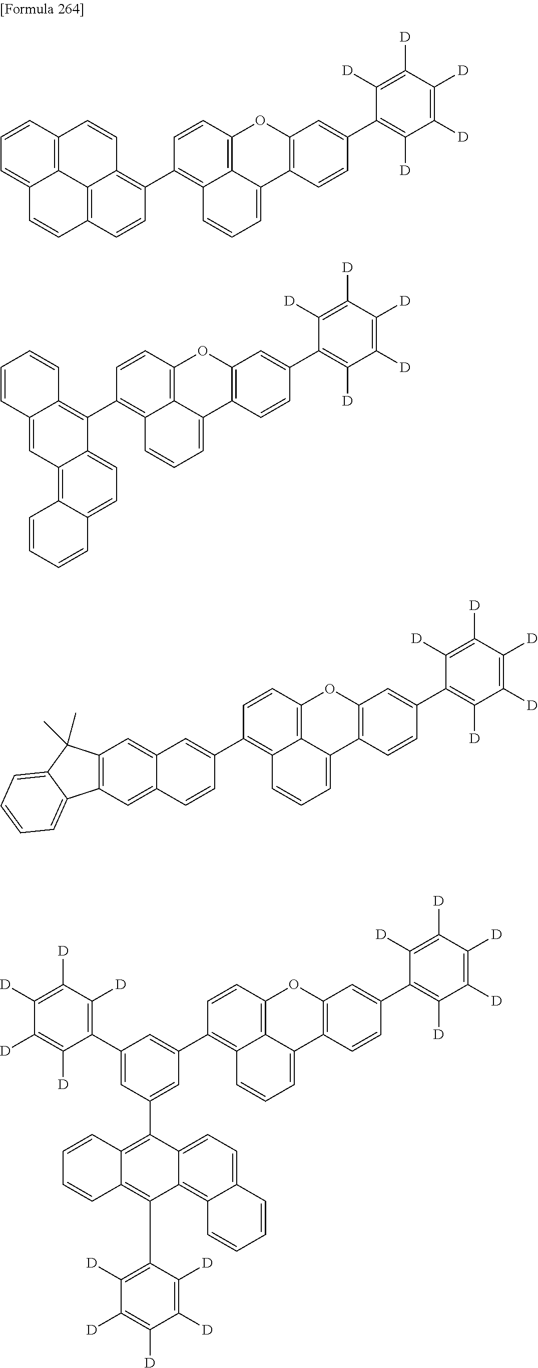 [SMILES: [2H]c1c([2H])c([2H])c(-c2ccc3c(c2)Oc2ccc(-c4ccc5ccc6cccc7ccc4c5c67)c4cccc-3c24)c([2H])c1[2H] ; [2H]c1c([2H])c([2H])c(-c2ccc3c(c2)Oc2ccc(-c4c5ccccc5cc5c4ccc4ccccc45)c4cccc-3c24)c([2H])c1[2H] ; [2H]c1c([2H])c([2H])c(-c2ccc3c(c2)Oc2ccc(-c4ccc5cc6c(cc5c4)C(C)(C)c4ccccc4-6)c4cccc-3c24)c([2H])c1[2H] ; [2H]c1c([2H])c([2H])c(-c2cc(-c3ccc4c5c(cccc35)-c3ccc(-c5c([2H])c([2H])c([2H])c([2H])c5[2H])cc3O4)cc(-c3c4ccccc4c(-c4c([2H])c([2H])c([2H])c([2H])c4[2H])c4c3ccc3ccccc34)c2)c([2H])c1[2H]]

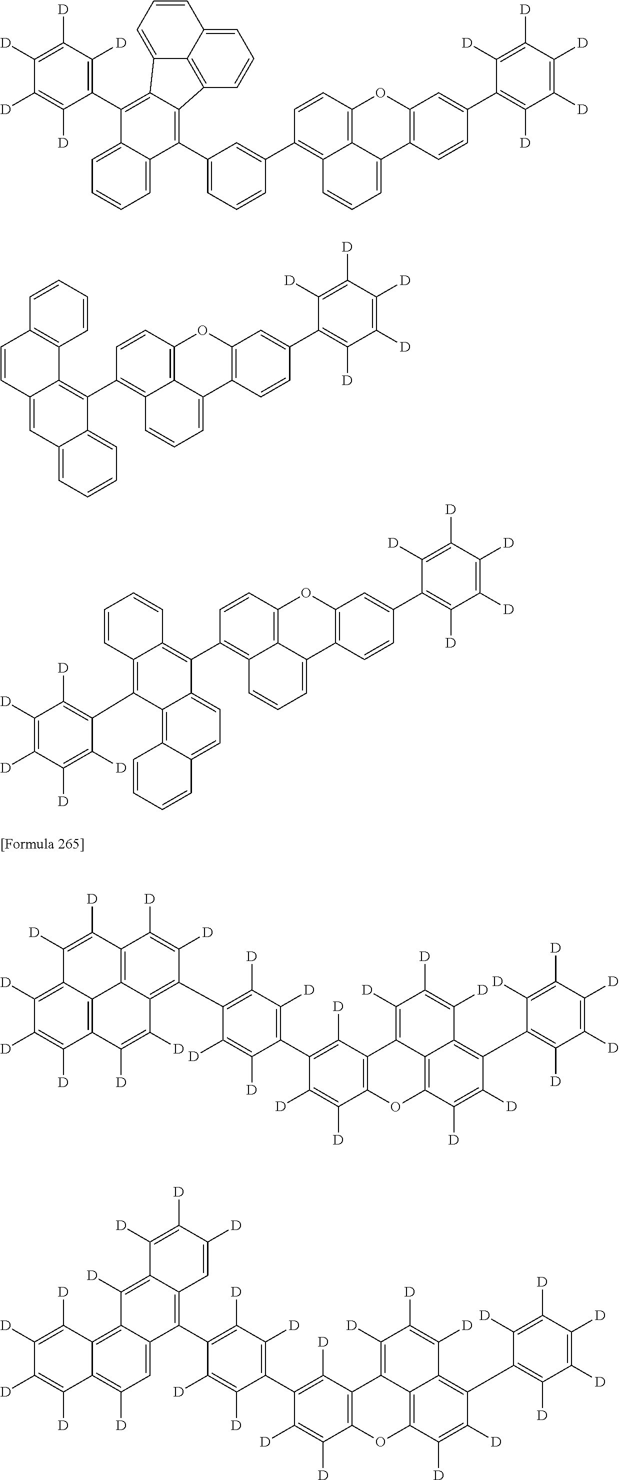

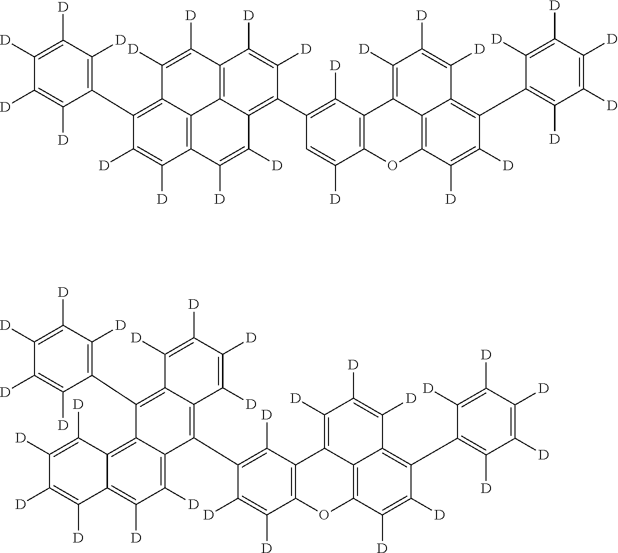 [SMILES: [2H]c1cc(-c2c([2H])c([2H])c3c([2H])c([2H])c4c(-c5c([2H])c([2H])c([2H])c([2H])c5[2H])c([2H])c([2H])c5c([2H])c([2H])c2c3c54)c([2H])c2c1Oc1c([2H])c([2H])c(-c3c([2H])c([2H])c([2H])c([2H])c3[2H])c3c([2H])c([2H])c([2H])c-2c13 ; [2H]c1c([2H])c([2H])c(-c2c([2H])c([2H])c3c4c(c([2H])c([2H])c([2H])c24)-c2c([2H])c(-c4c5c([2H])c([2H])c([2H])c([2H])c5c(-c5c([2H])c([2H])c([2H])c([2H])c5[2H])c5c4c([2H])c([2H])c4c([2H])c([2H])c([2H])c([2H])c45)c([2H])c([2H])c2O3)c([2H])c1[2H]]

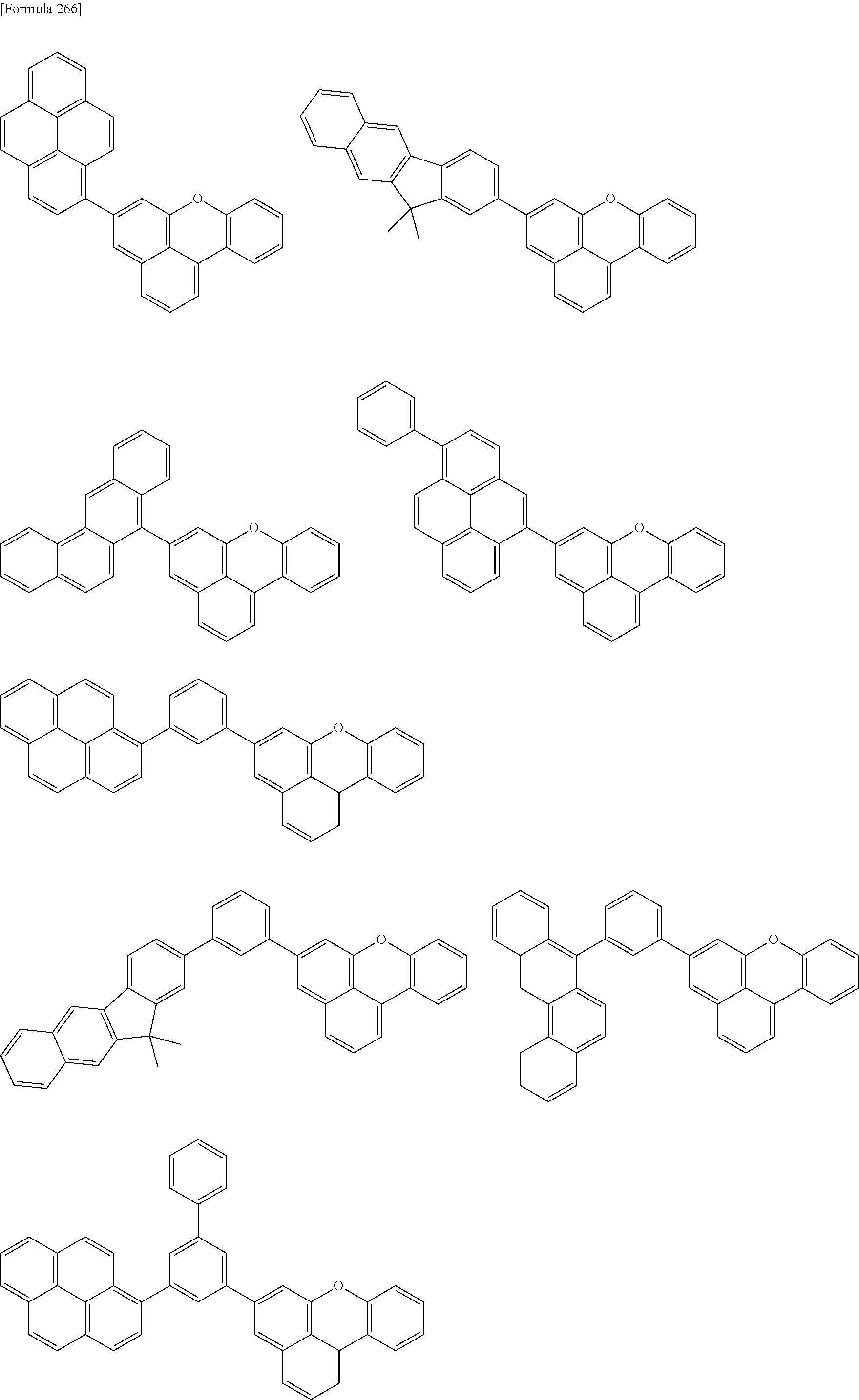

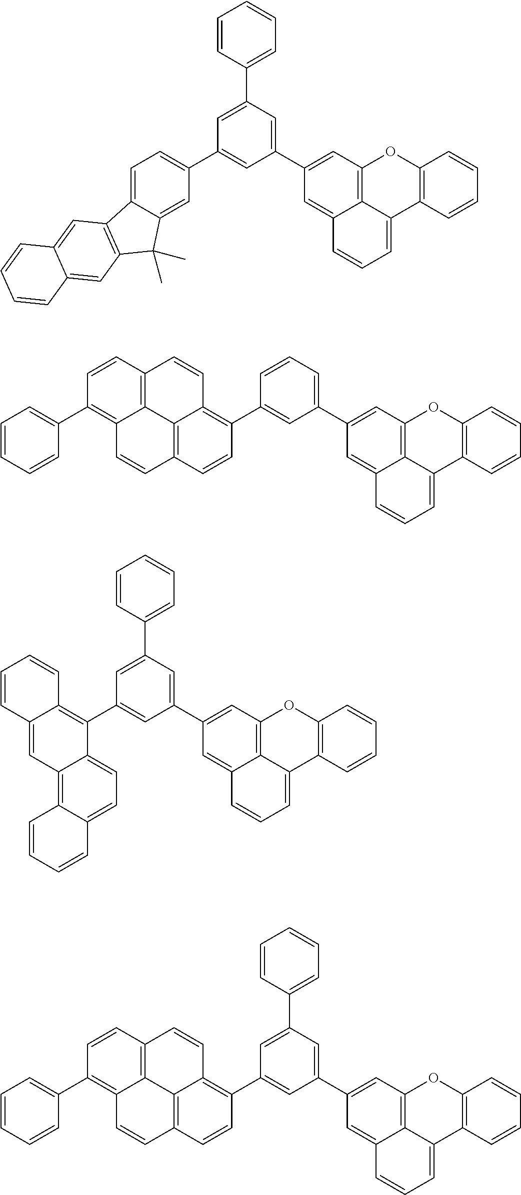 [SMILES: CC1(C)c2cc(-c3cc(-c4ccccc4)cc(-c4cc5c6c(cccc6c4)-c4ccccc4O5)c3)ccc2-c2cc3ccccc3cc21 ; c1ccc(-c2ccc3ccc4c(-c5cccc(-c6cc7c8c(cccc8c6)-c6ccccc6O7)c5)ccc5ccc2c3c54)cc1 ; c1ccc(-c2cc(-c3cc4c5c(cccc5c3)-c3ccccc3O4)cc(-c3c4ccccc4cc4c3ccc3ccccc34)c2)cc1 ; c1ccc(-c2cc(-c3cc4c5c(cccc5c3)-c3ccccc3O4)cc(-c3ccc4ccc5c(-c6ccccc6)ccc6ccc3c4c65)c2)cc1]

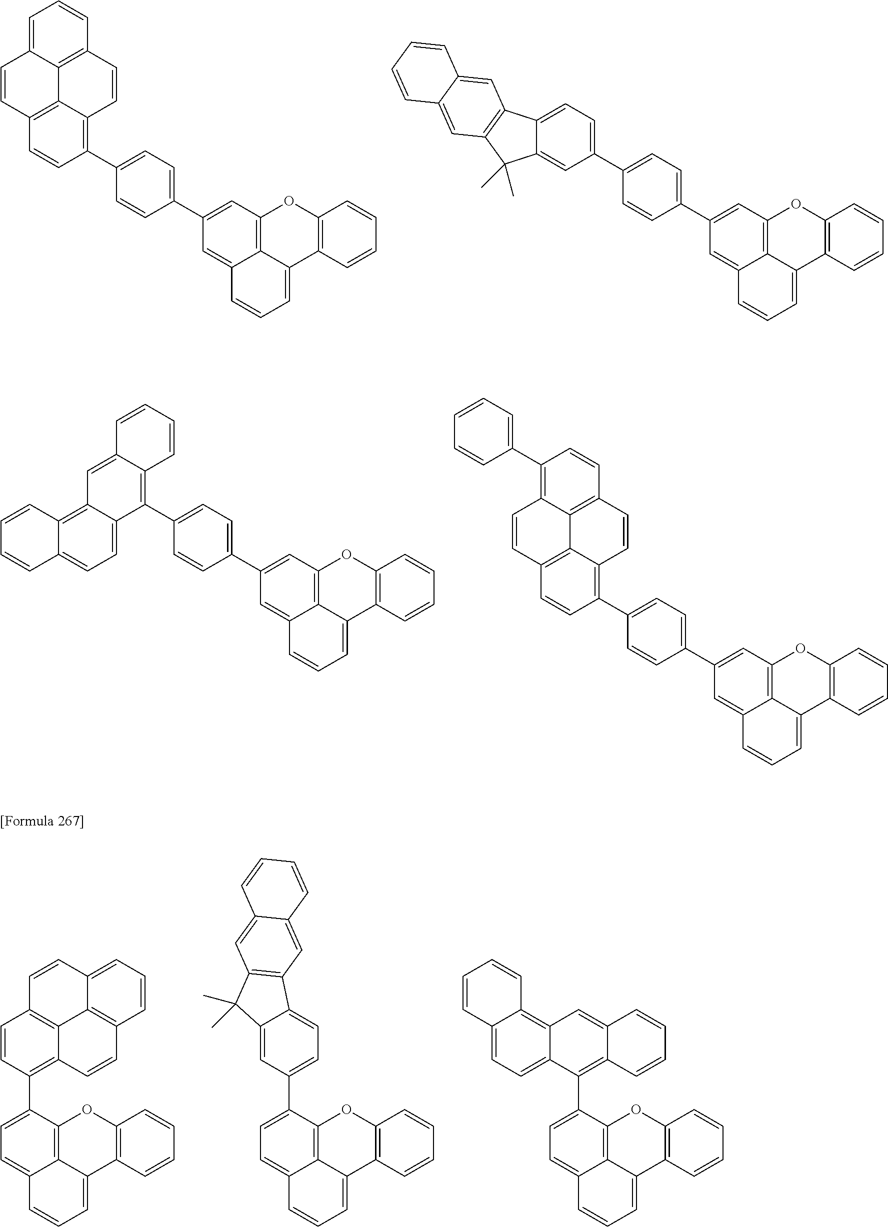

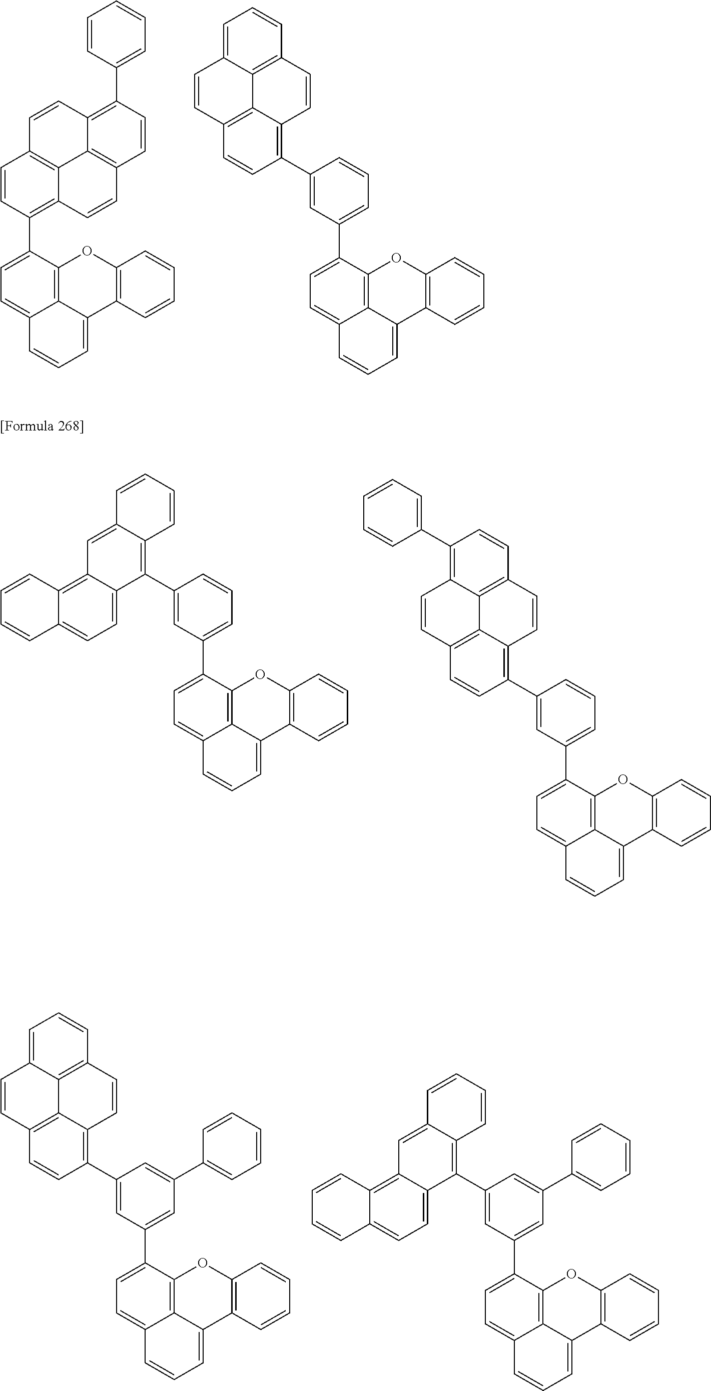 [SMILES: c1ccc(-c2ccc3ccc4c(-c5ccc6cccc7c6c5Oc5ccccc5-7)ccc5ccc2c3c54)cc1 ; c1cc(-c2ccc3cccc4c3c2Oc2ccccc2-4)cc(-c2ccc3ccc4cccc5ccc2c3c45)c1 ; c1cc(-c2ccc3cccc4c3c2Oc2ccccc2-4)cc(-c2c3ccccc3cc3c2ccc2ccccc23)c1 ; c1ccc(-c2ccc3ccc4c(-c5cccc(-c6ccc7cccc8c7c6Oc6ccccc6-8)c5)ccc5ccc2c3c54)cc1 ; c1ccc(-c2cc(-c3ccc4cccc5c4c3Oc3ccccc3-5)cc(-c3ccc4ccc5cccc6ccc3c4c56)c2)cc1 ; c1ccc(-c2cc(-c3ccc4cccc5c4c3Oc3ccccc3-5)cc(-c3c4ccccc4cc4c3ccc3ccccc34)c2)cc1]

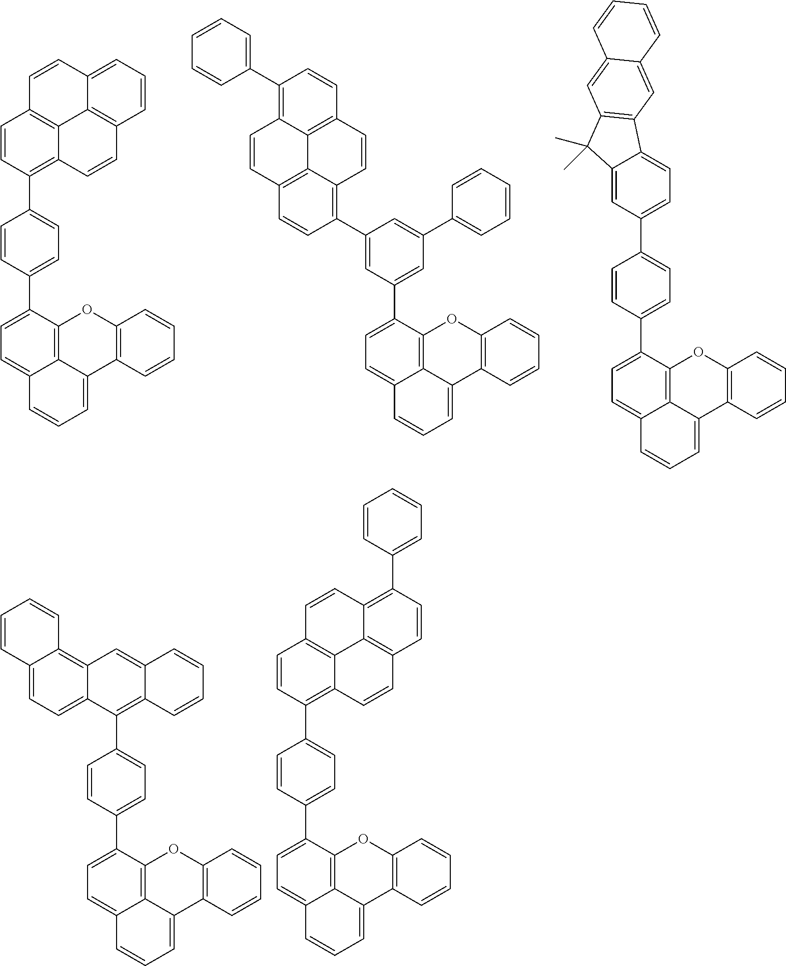

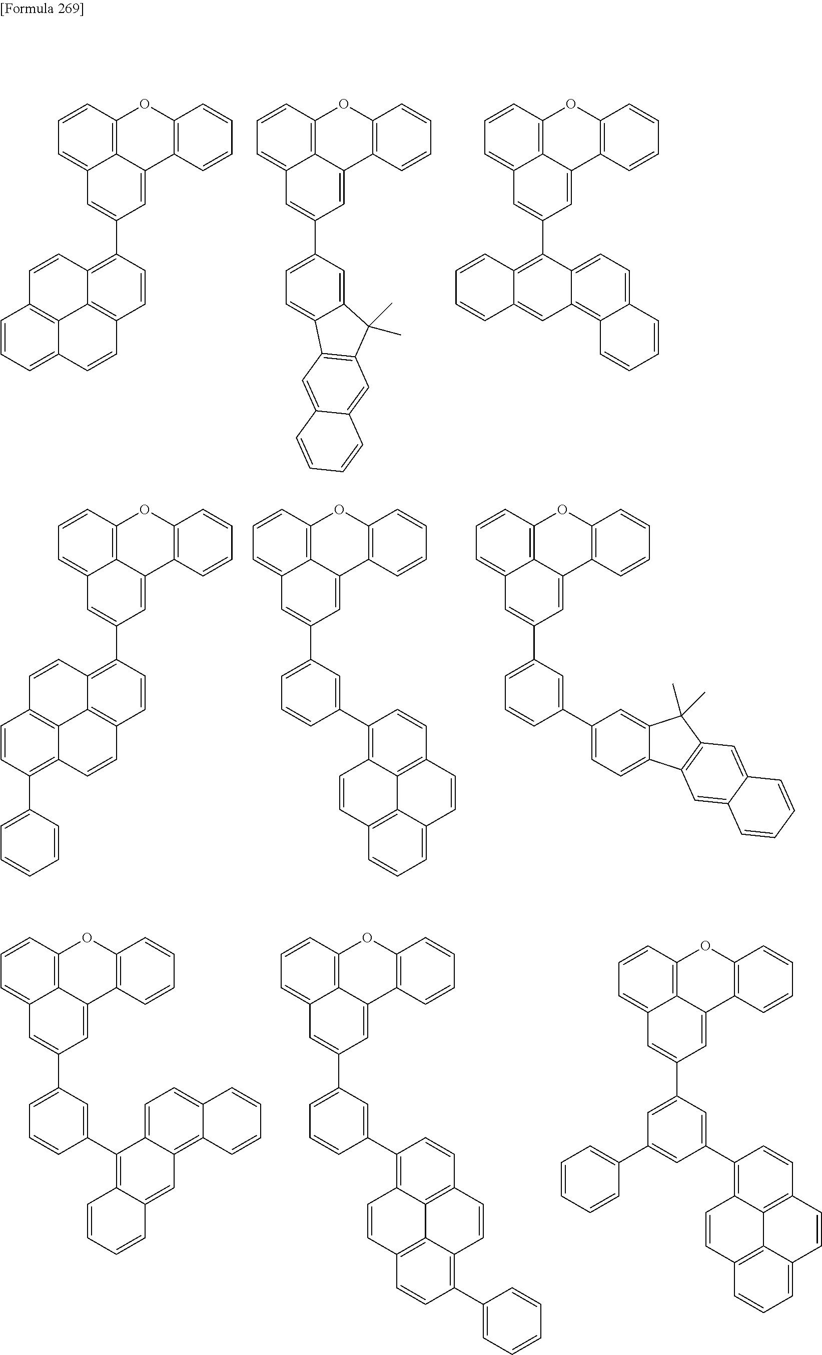

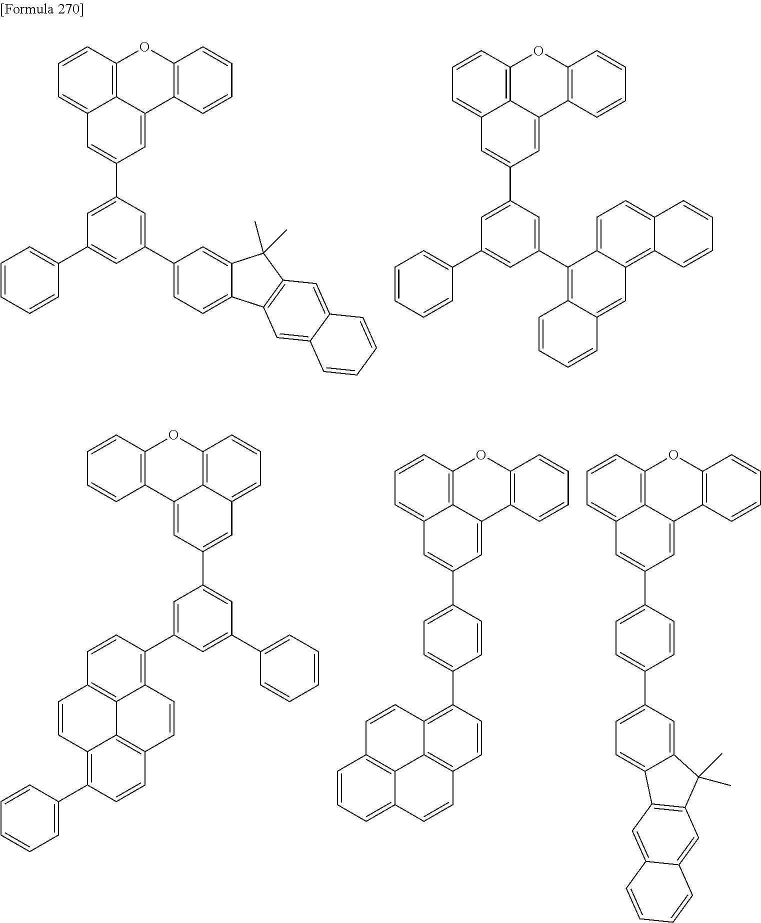

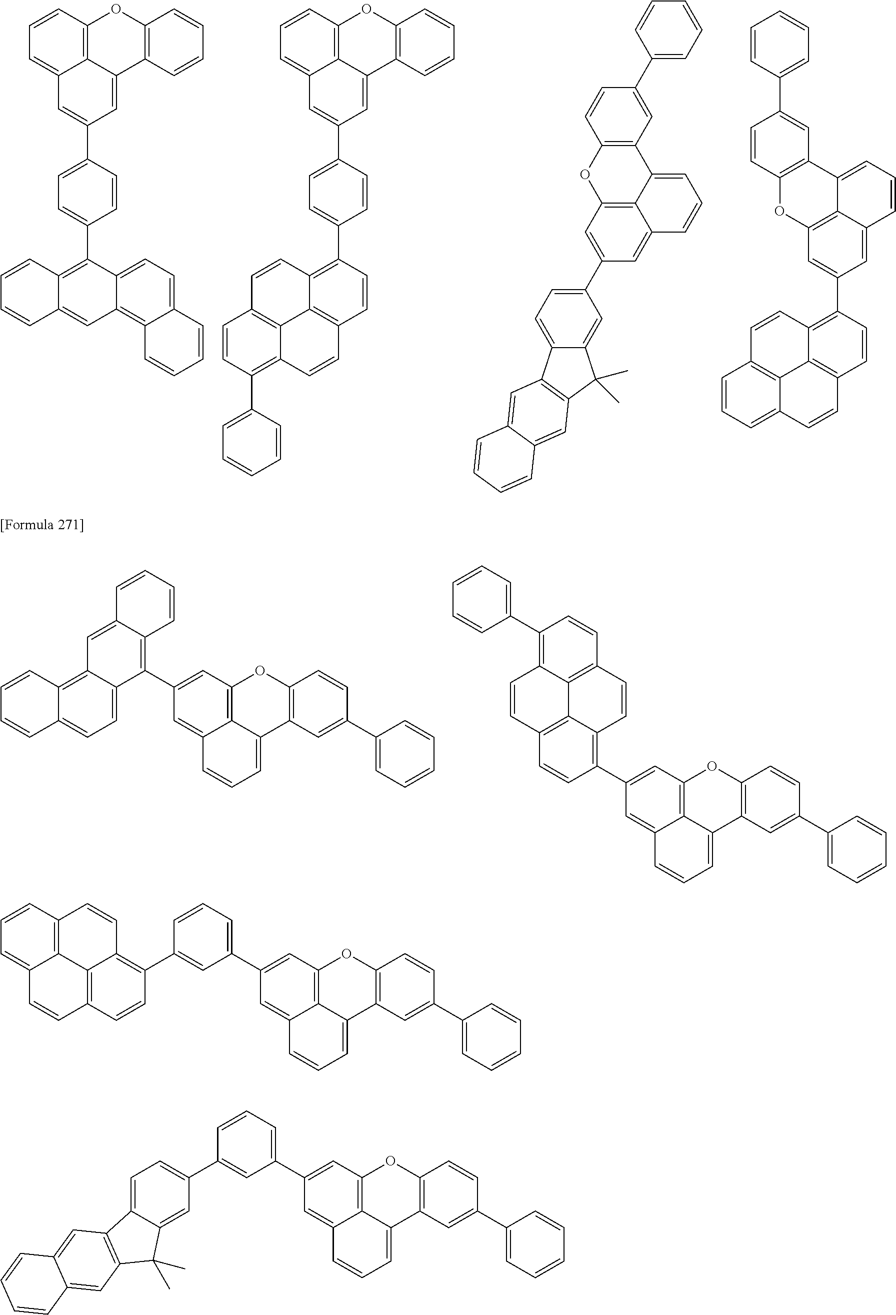

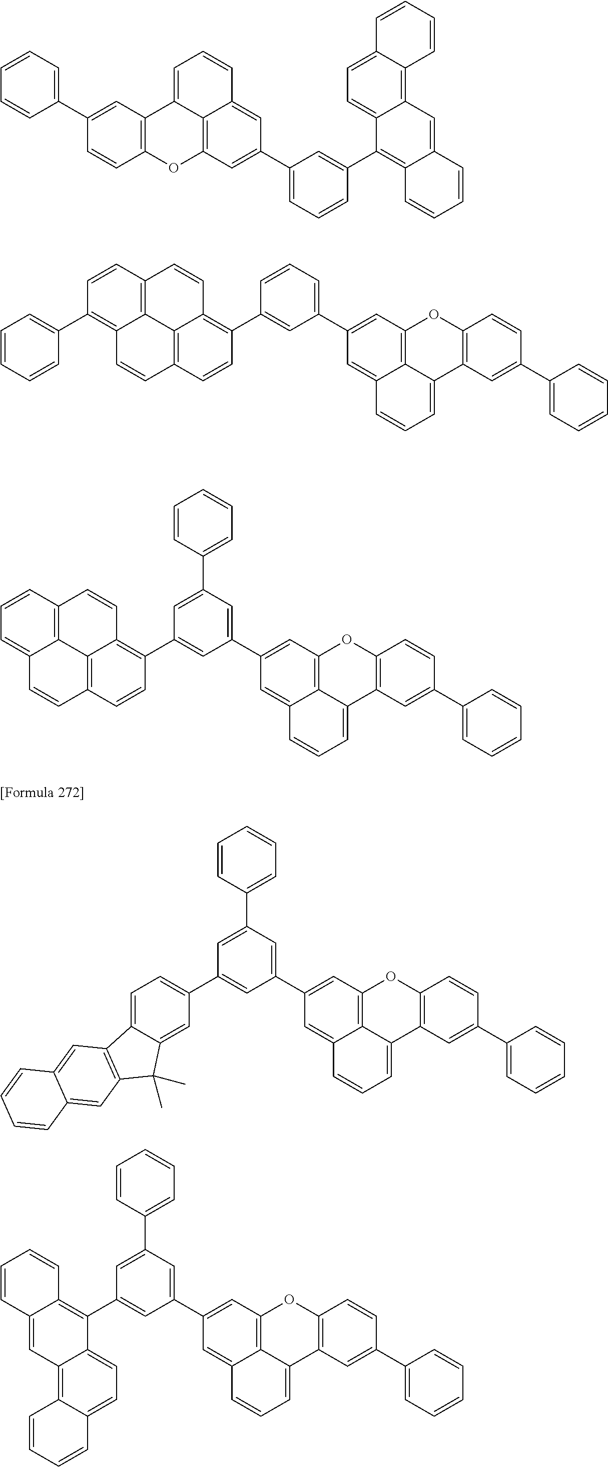

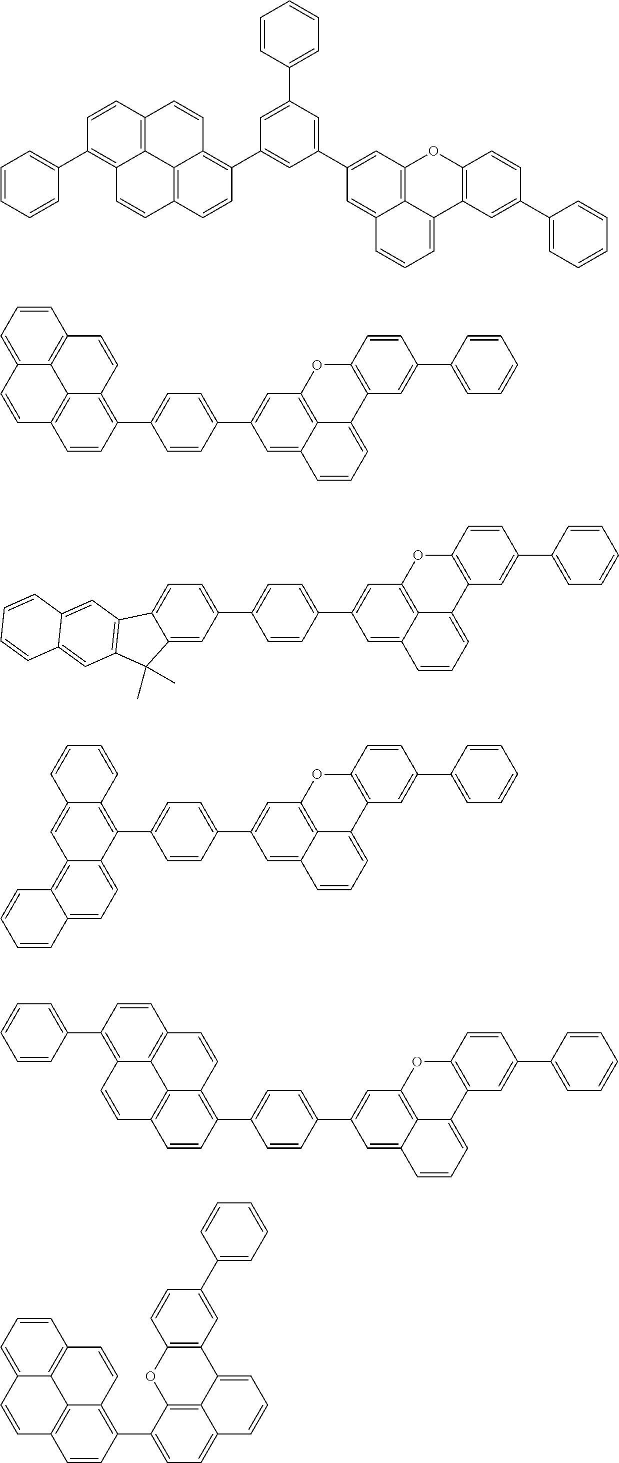

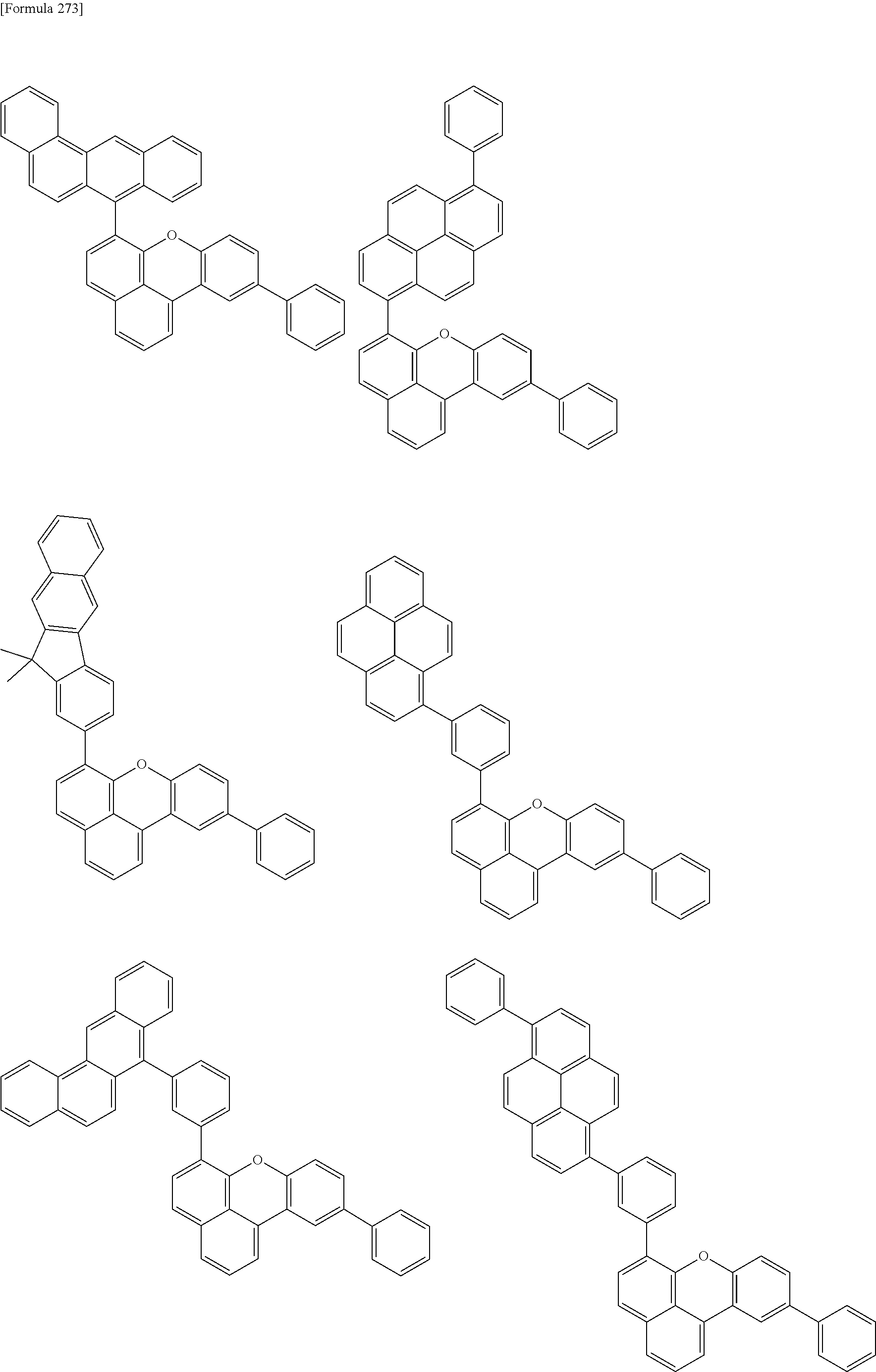

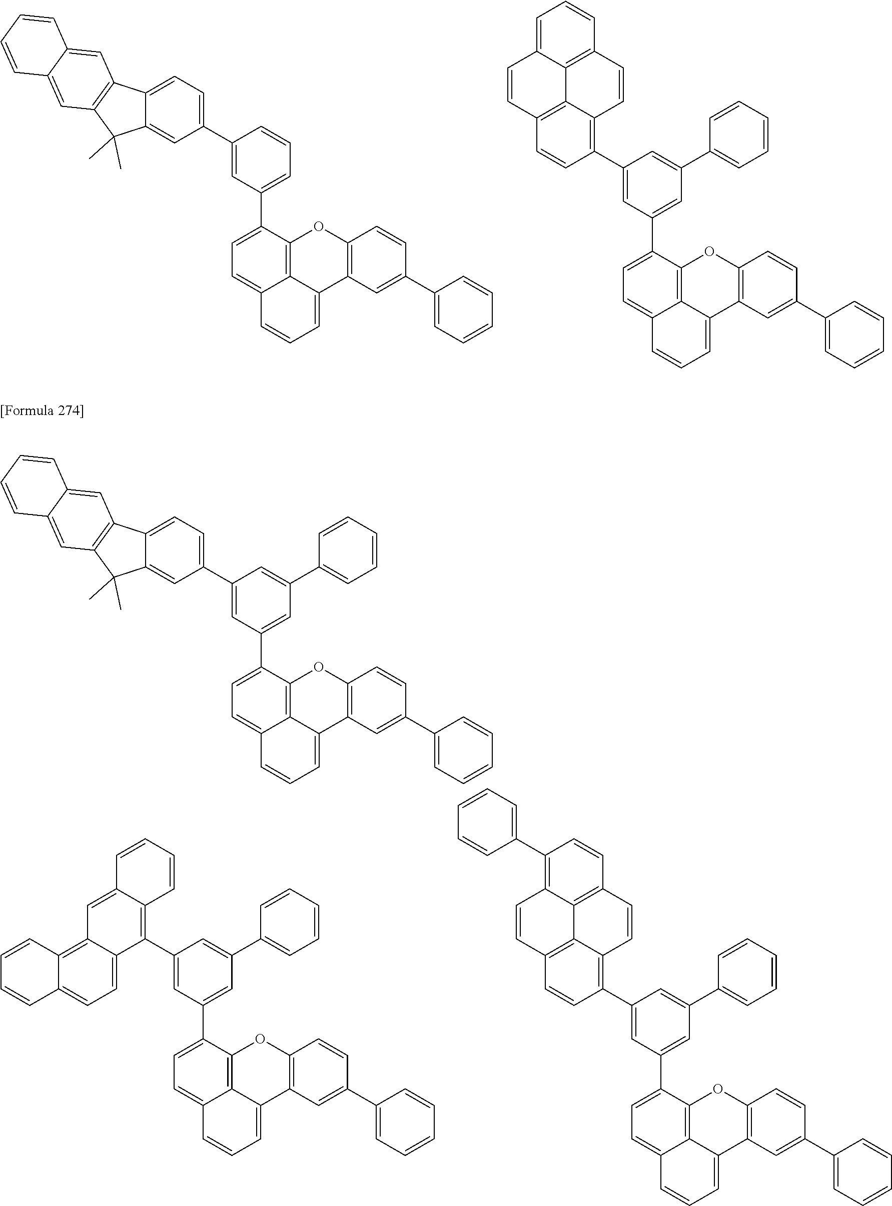

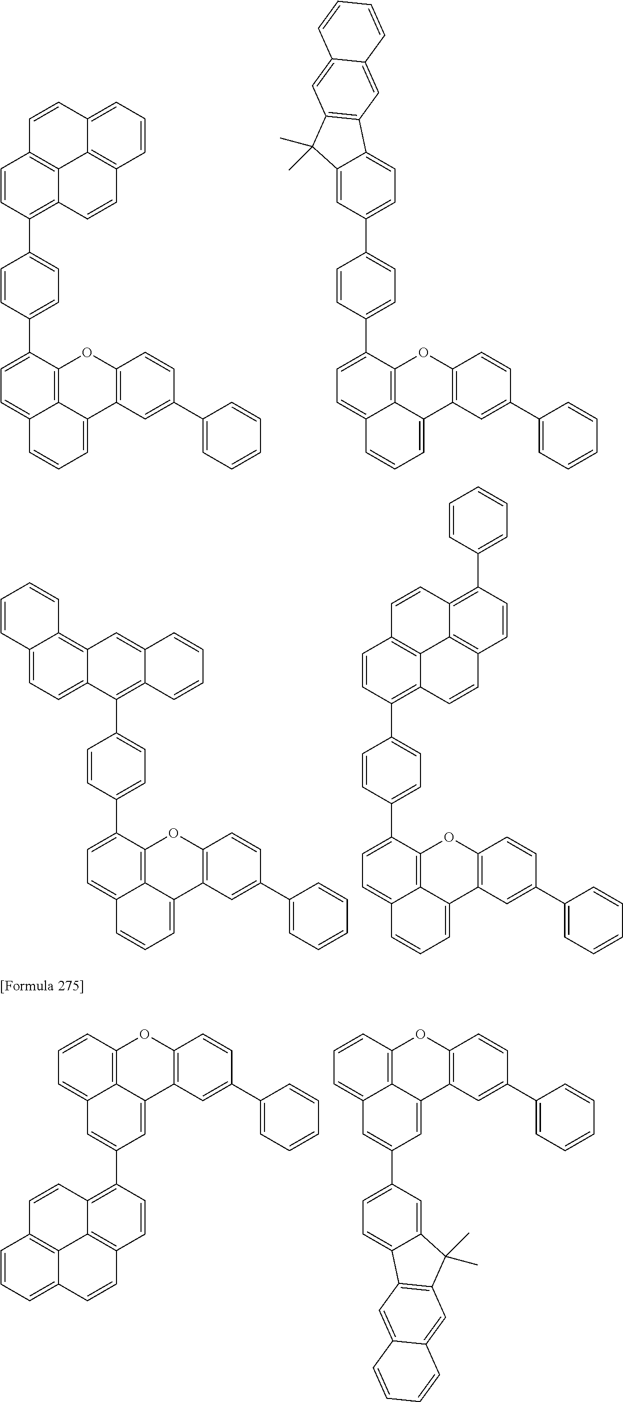

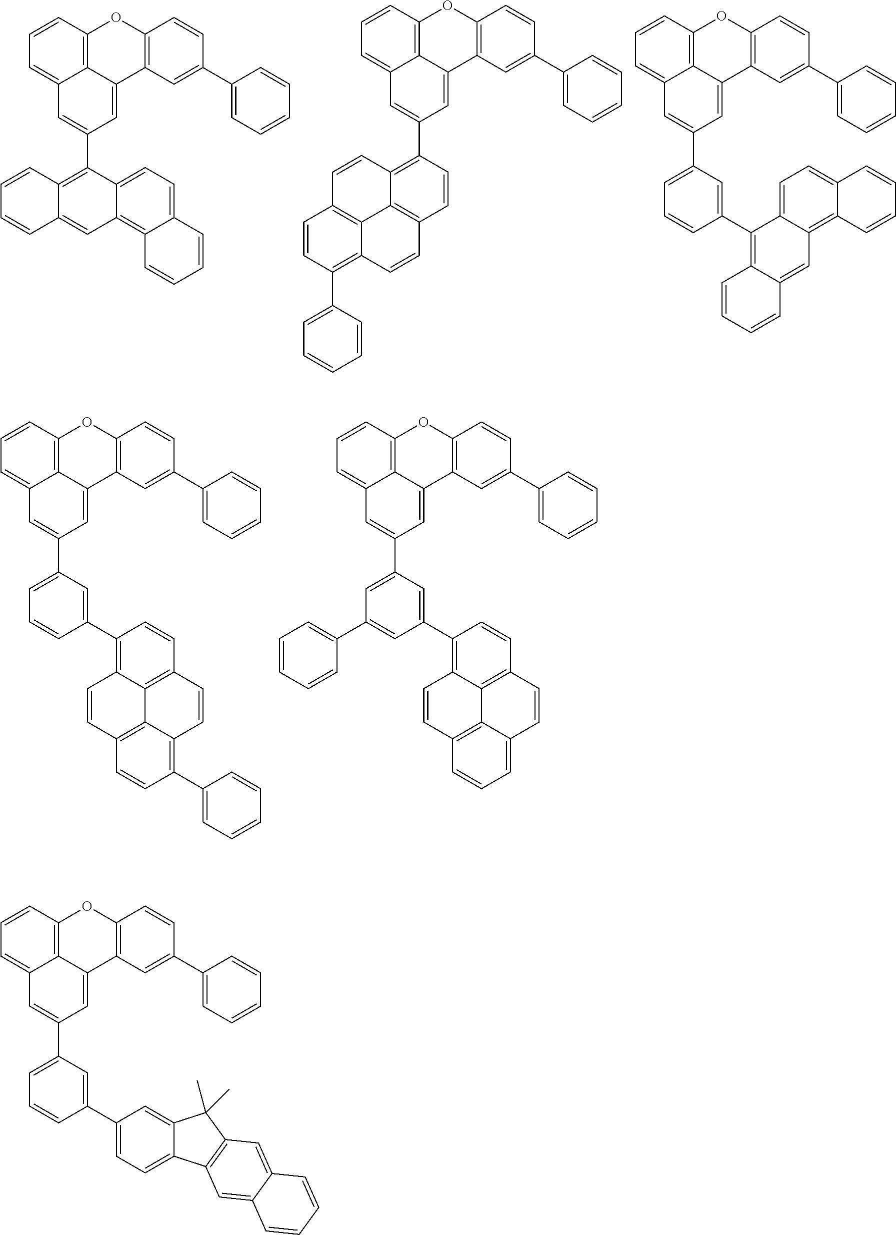

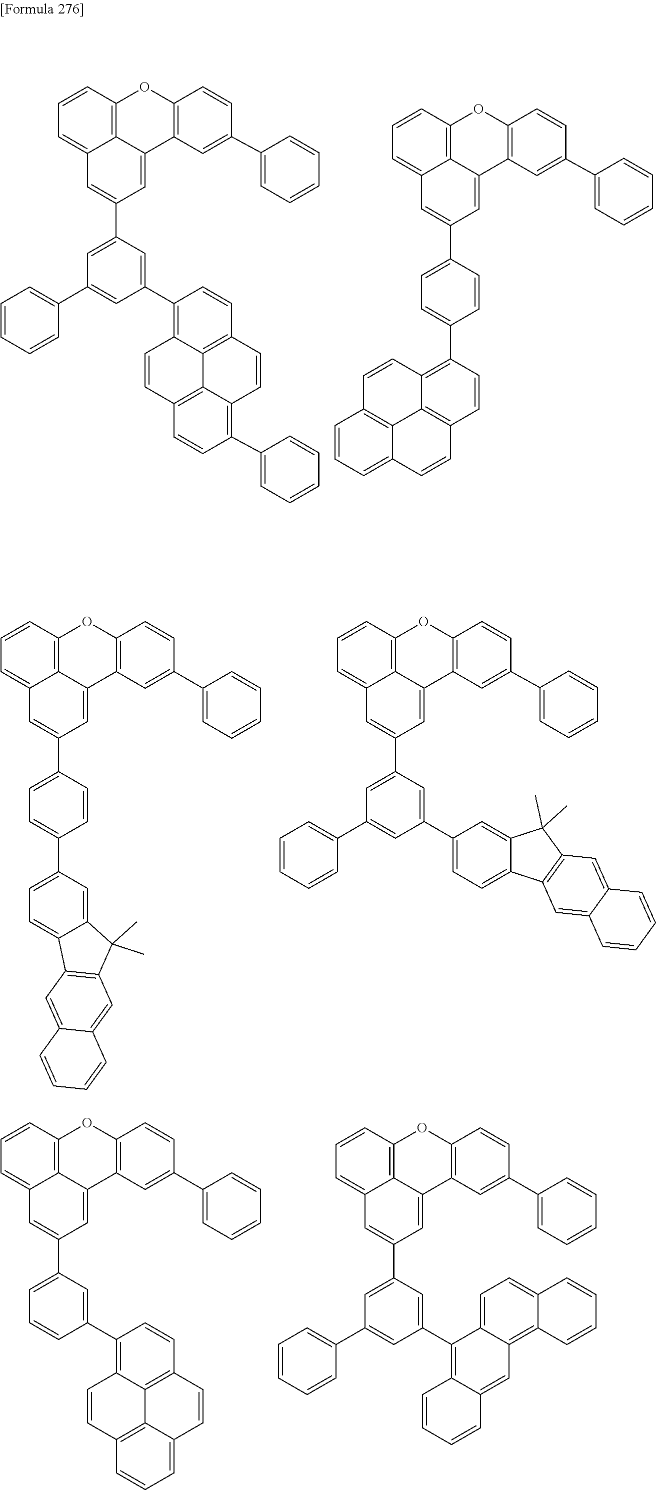

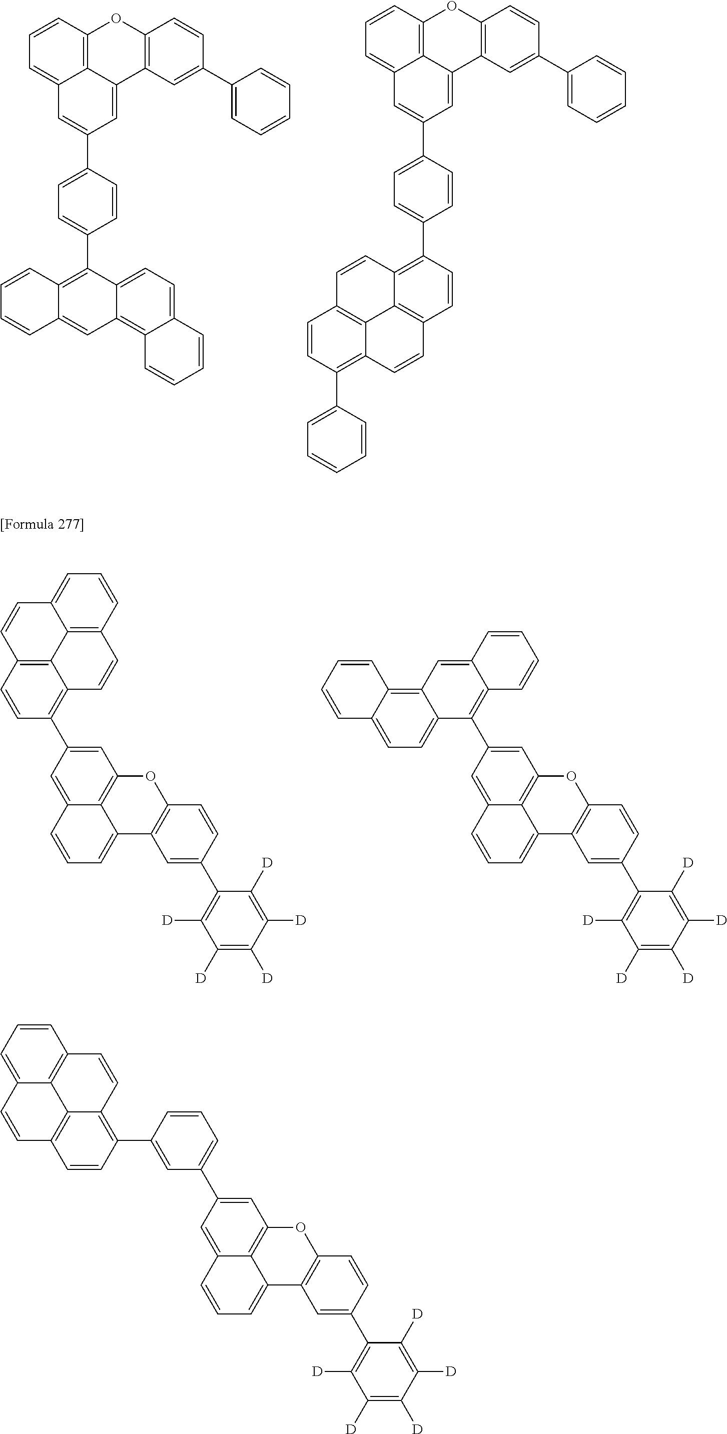 [SMILES: c1ccc(-c2ccc3c(c2)-c2cc(-c4ccc(-c5c6ccccc6cc6c5ccc5ccccc56)cc4)cc4cccc(c24)O3)cc1 ; c1ccc(-c2ccc3c(c2)-c2cc(-c4ccc(-c5ccc6ccc7c(-c8ccccc8)ccc8ccc5c6c87)cc4)cc4cccc(c24)O3)cc1 ; [2H]c1c([2H])c([2H])c(-c2ccc3c(c2)-c2cccc4cc(-c5ccc6ccc7cccc8ccc5c6c78)cc(c24)O3)c([2H])c1[2H] ; [2H]c1c([2H])c([2H])c(-c2ccc3c(c2)-c2cccc4cc(-c5c6ccccc6cc6c5ccc5ccccc56)cc(c24)O3)c([2H])c1[2H] ; [2H]c1c([2H])c([2H])c(-c2ccc3c(c2)-c2cccc4cc(-c5cccc(-c6ccc7ccc8cccc9ccc6c7c89)c5)cc(c24)O3)c([2H])c1[2H]]

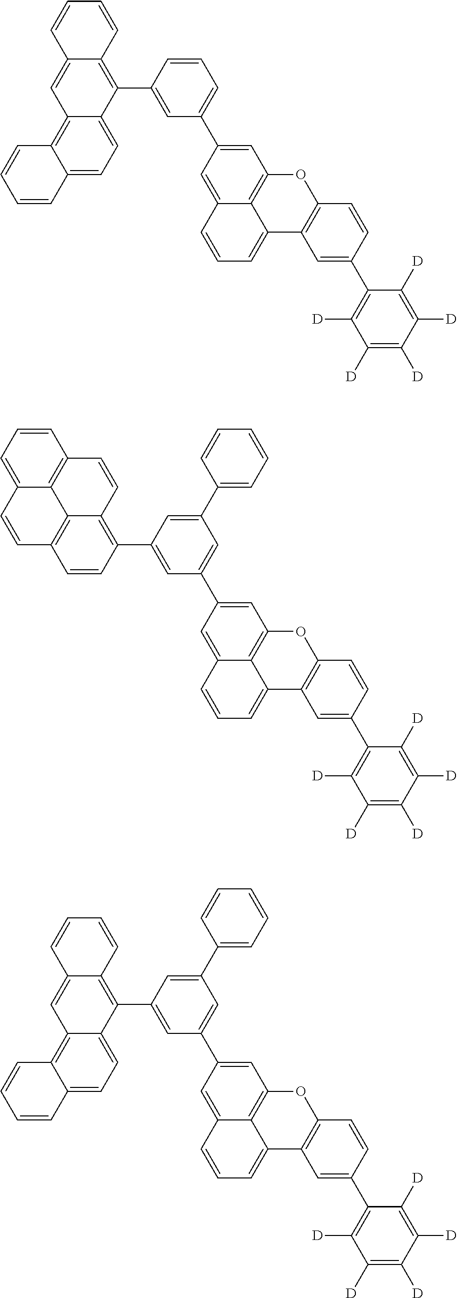 [SMILES: [2H]c1c([2H])c([2H])c(-c2ccc3c(c2)-c2cccc4cc(-c5cccc(-c6c7ccccc7cc7c6ccc6ccccc67)c5)cc(c24)O3)c([2H])c1[2H] ; [2H]c1c([2H])c([2H])c(-c2ccc3c(c2)-c2cccc4cc(-c5cc(-c6ccccc6)cc(-c6ccc7ccc8cccc9ccc6c7c89)c5)cc(c24)O3)c([2H])c1[2H] ; [2H]c1c([2H])c([2H])c(-c2ccc3c(c2)-c2cccc4cc(-c5cc(-c6ccccc6)cc(-c6c7ccccc7cc7c6ccc6ccccc67)c5)cc(c24)O3)c([2H])c1[2H]]

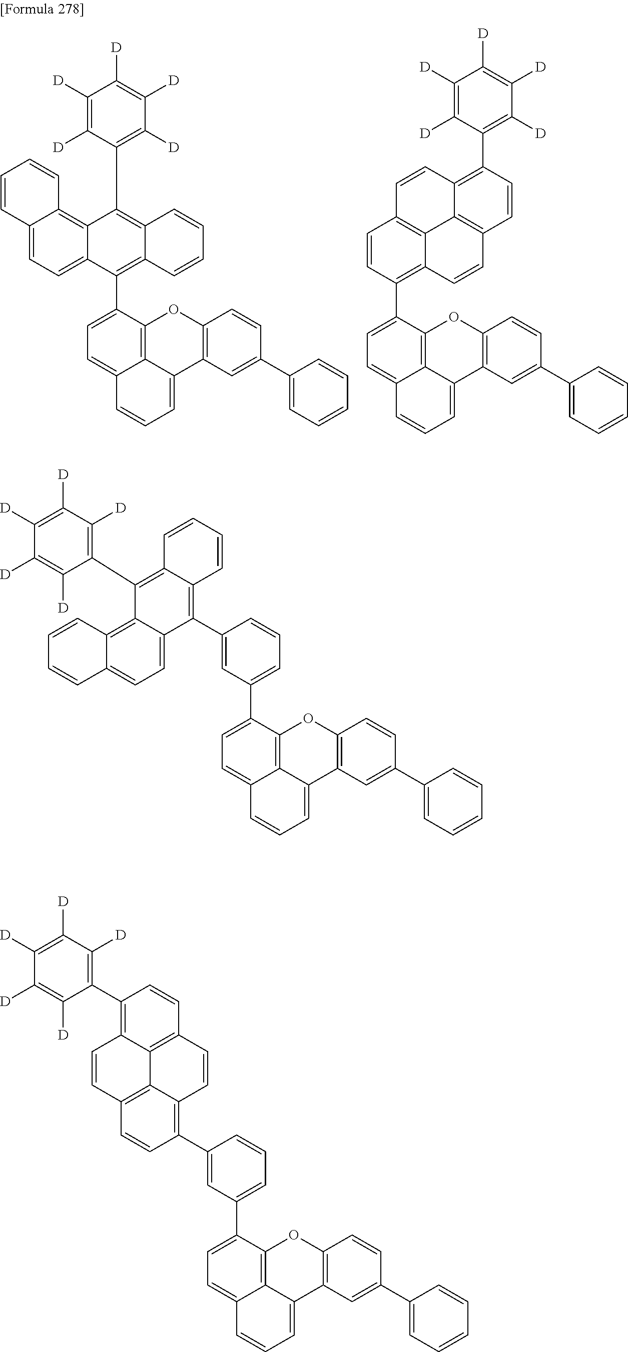

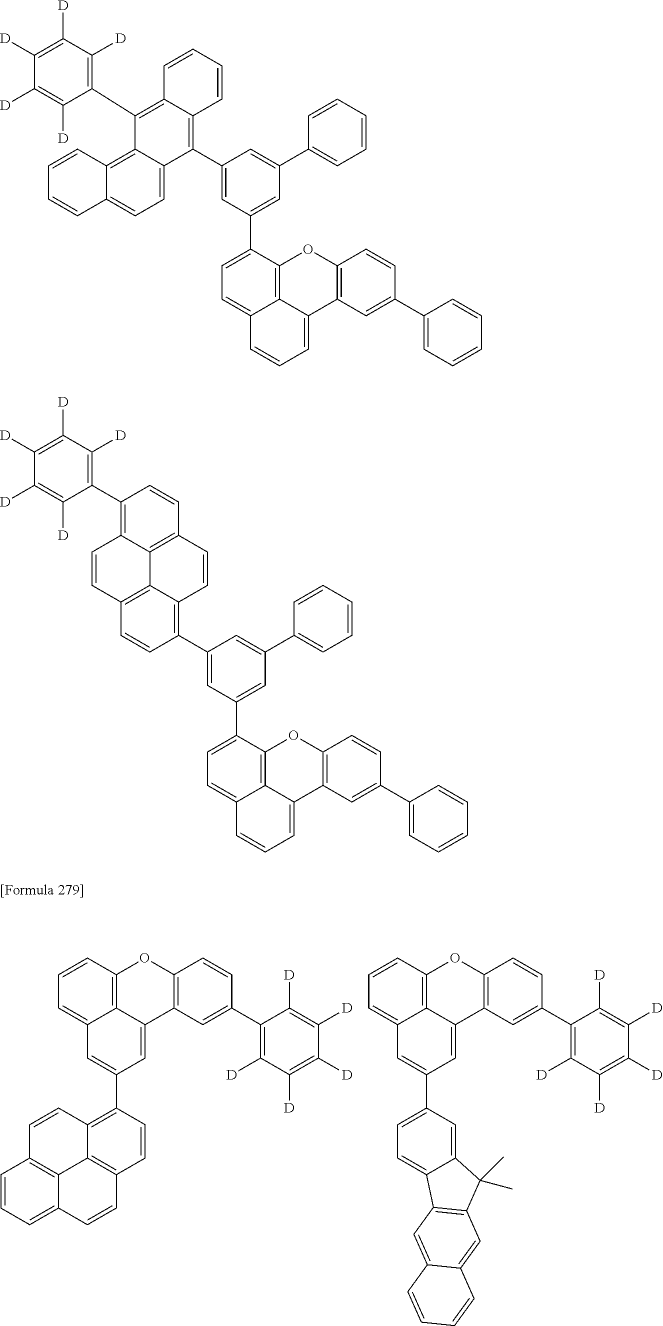

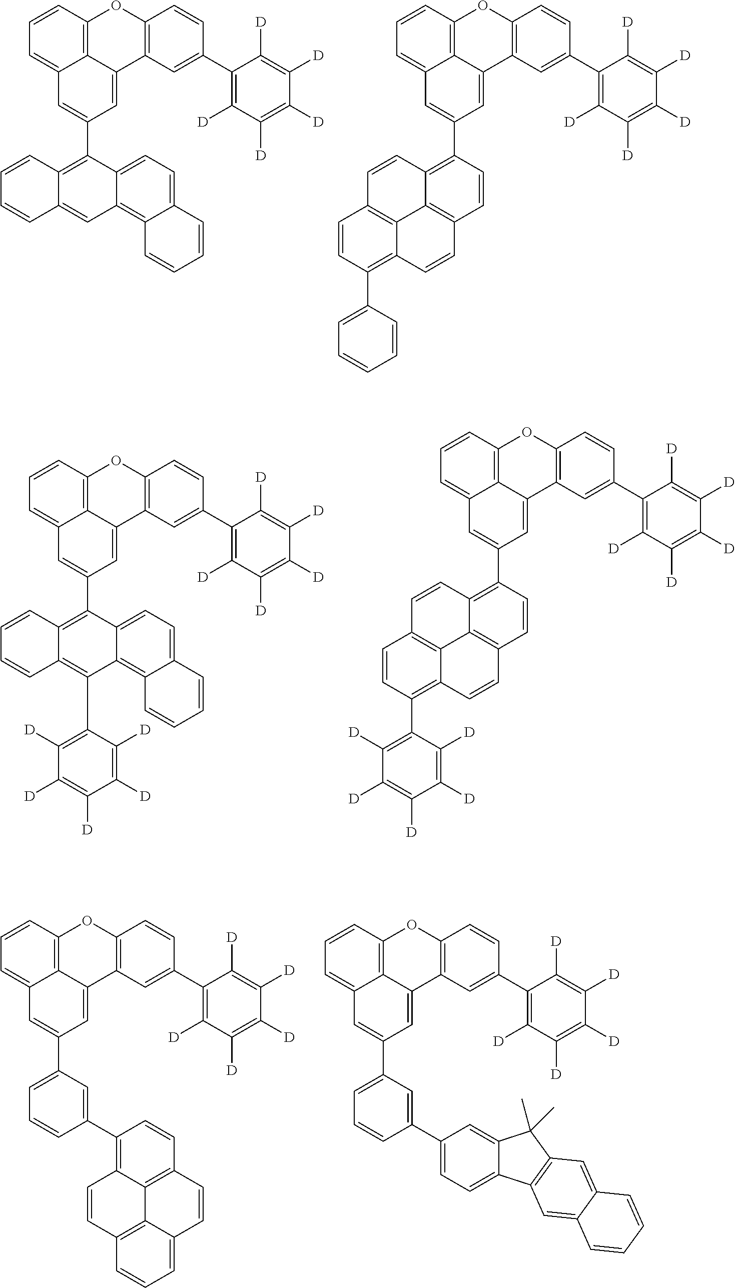

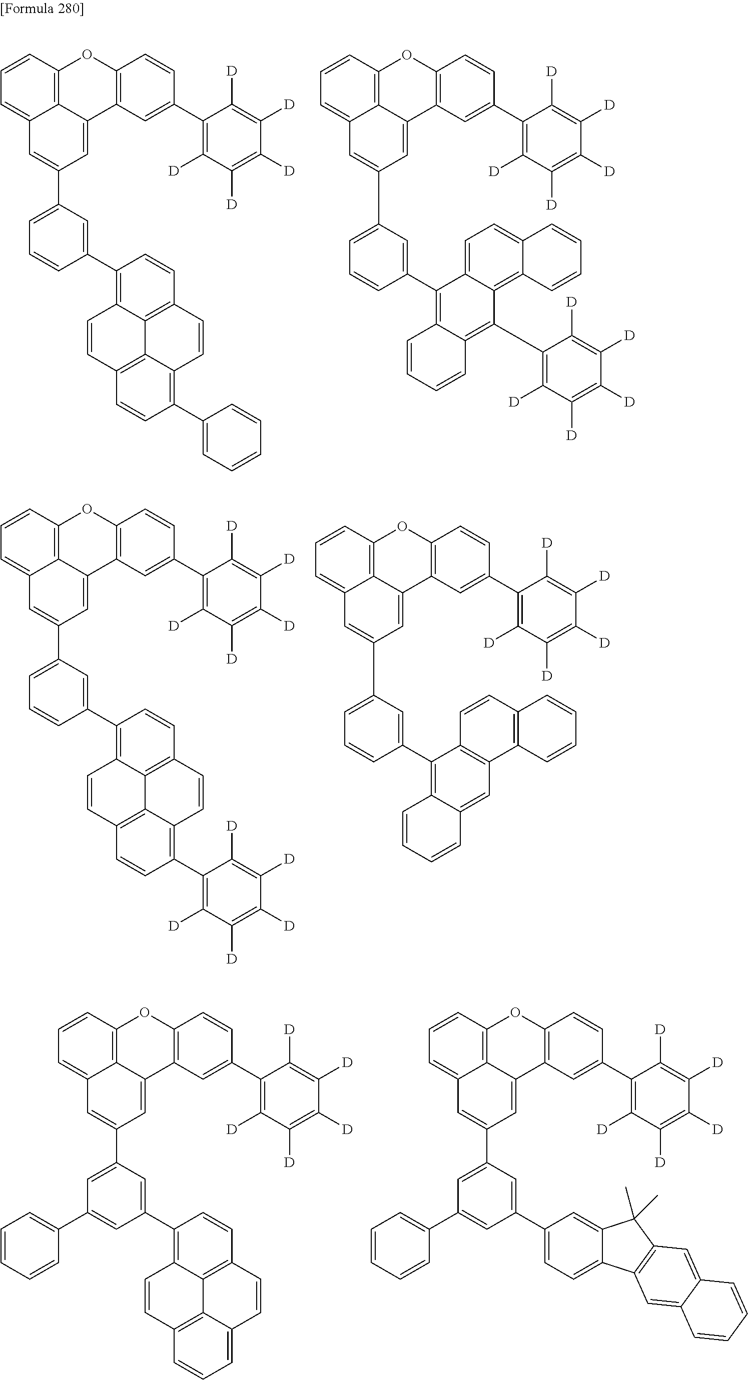

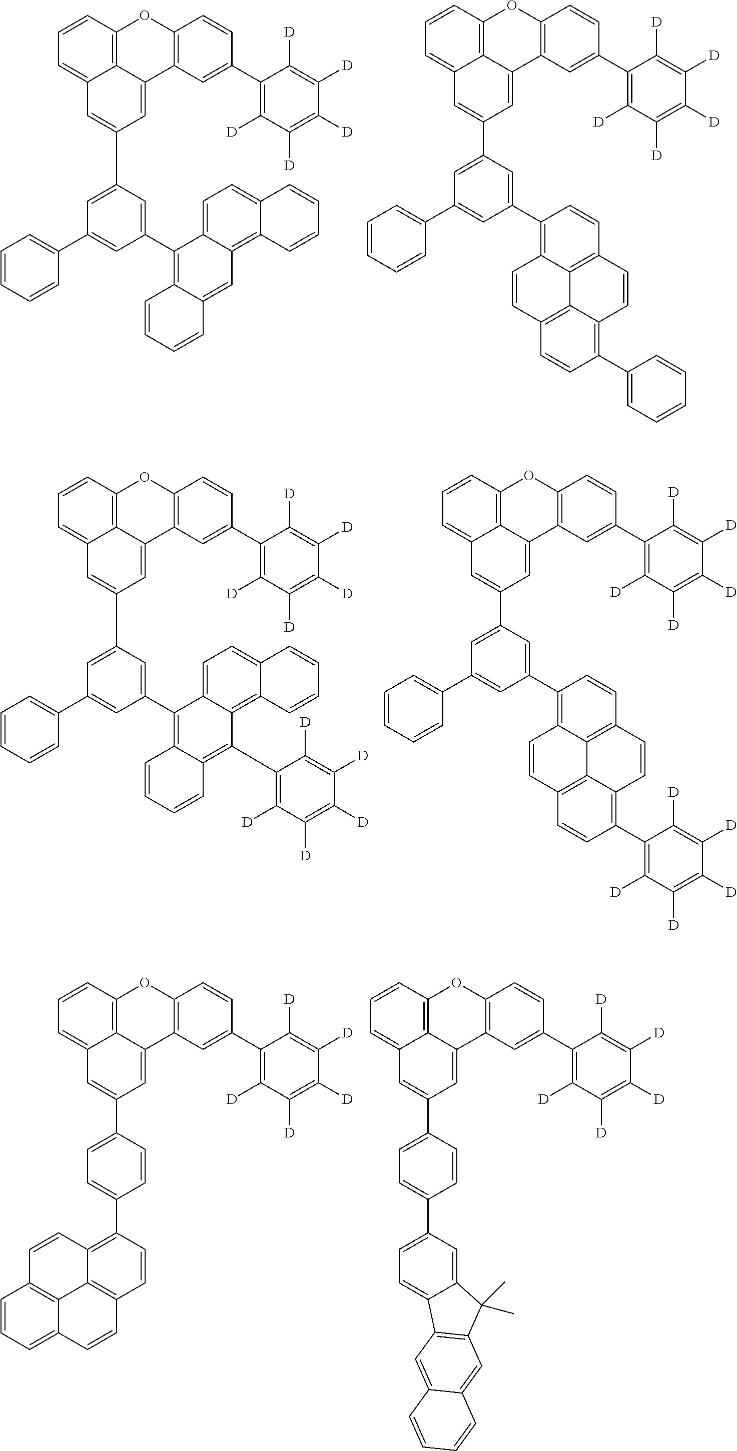

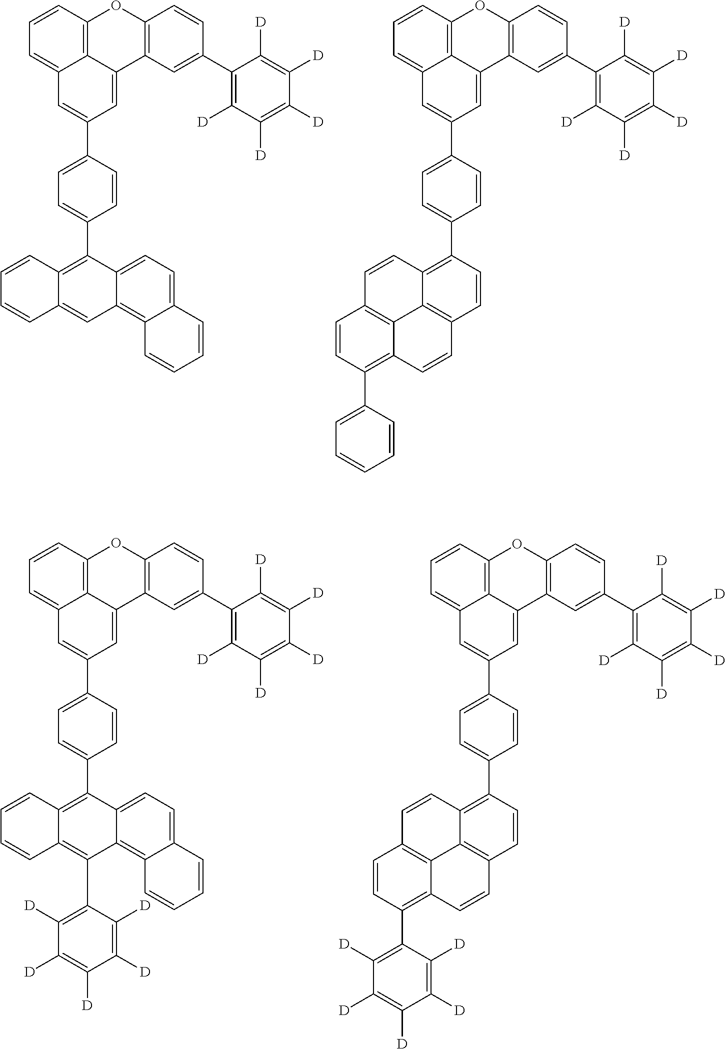

The compound according to the exemplary embodiment can improve performance of an organic EL device. Moreover, when the compound according to the exemplary embodiment is used in an emitting layer (first emitting layer) close to an anode of an organic EL device in which a plurality of emitting layers are layered, the organic EL device in a favorable balance between a luminous efficiency and a lifetime can be expected to be provided.

### Second Exemplary Embodiment

Organic-Electroluminescence-Device Material

An organic-electroluminescence-device material according to a second exemplary embodiment contains the compound according to the first exemplary embodiment. As one example, the organic-electroluminescence-device material contains only the compound according to the first exemplary embodiment. As another example, the organic-electroluminescence-device material contains the compound according to the first exemplary embodiment and another compound(s) different from the compound according to the first exemplary embodiment.

In the organic-electroluminescence-device material according to the second exemplary embodiment, the compound according to the first exemplary embodiment is preferably a host material. In this case, the organic-electroluminescence-device material may contain the compound according to the first exemplary embodiment as the host material and another compound(s) such as a dopant material.

### Third Exemplary Embodiment

Organic Electroluminescence Device

An organic EL device according to a third exemplary embodiment will be described.

The organic EL device according to the third exemplary embodiment includes an anode, a cathode, and at least one organic layer disposed between the anode and the cathode. The organic layer includes at least one layer formed of an organic compound. Alternatively, the organic layer includes a plurality of layers formed of an organic compound(s). The organic layer may further contain an inorganic compound.

In the organic EL device according to the third exemplary embodiment, at least one of the at least one organic layer contains the compound according to the first exemplary embodiment.

In the organic EL device according to the third exemplary embodiment, at least one of the at least one organic layer is preferably an emitting layer. In the organic EL device according to the third exemplary embodiment, the emitting layer preferably contains the compound according to the first exemplary embodiment. In an exemplary embodiment, the emitting layer contains a compound represented by the formula (100). In an exemplary embodiment, the emitting layer contains a compound represented by the formula (1). In an exemplary embodiment, the emitting layer contains a compound represented by the formula (100A).

In an exemplary embodiment, the emitting layer may contain a metal complex.

In an exemplary embodiment, the emitting layer preferably does not contain a metal complex.

In an exemplary embodiment, the emitting layer preferably does not contain a phosphorescent material (dopant material).

In an exemplary embodiment, the emitting layer preferably does not contain a heavy metal complex and a phosphorescent rare-metal complex. Examples of the heavy metal complex include an iridium complex, osmium complex, and platinum complex.

For instance, the organic layer may consist of a single emitting layer or may further include at least one layer usable in organic EL devices. Examples of the layer usable in the organic EL device, which are not particularly limited, include at least one layer selected from the group consisting of a hole injecting layer, hole transporting layer, electron injecting layer, electron transporting layer, and blocking layer.

The organic EL device according to the exemplary embodiment preferably includes a hole transporting layer between the anode and the emitting layer.

The organic EL device according to the exemplary embodiment preferably includes an electron transporting layer between the cathode and the emitting layer.

In the organic EL device according to the exemplary embodiment, the organic layer may be provided by a plurality of emitting layers. For instance, the organic layer may be provided by two emitting layers, that is, a first emitting layer and a second emitting layer. The organic EL device according to the exemplary embodiment may include at least one organic layer in addition to the first and second emitting layers. For instance, the organic EL device according to the exemplary embodiment may further include at least one layer selected from the group consisting of a hole injecting layer, hole transporting layer, electron injecting layer, electron transporting layer, hole blocking layer, and electron blocking layer.

In the organic EL device according to the exemplary embodiment, the organic layer also preferably has the first and second emitting layers.

An exemplary arrangement is assumed in which the first emitting layer is disposed between the anode and the cathode and the second emitting layer is disposed between the first emitting layer and the cathode. Another exemplary arrangement is assumed in which the second emitting layer is disposed between the anode and the cathode and the first emitting layer is disposed between the second emitting layer and the cathode. In the organic EL device according to the exemplary embodiment, the first and second emitting layers can be disposed in any one of the above exemplary arrangements.

In the organic EL device according to the exemplary embodiment, the first and second emitting layers may be in direct contact with each other or may not be in direct contact with each other.

An organic EL device according to an exemplary embodiment includes: the anode, the cathode, the first emitting layer disposed between the anode and the cathode, and the second emitting layer disposed between the first emitting layer and the cathode, in which the first emitting layer contains a first compound, the first compound is a compound represented by the formula (100) and having at least one group represented by the formula (110), and the first and second emitting layers are in direct contact with each other.

In a case where the organic layer has the emitting layer in the organic EL device according to the exemplary embodiment, the organic EL device preferably has the electron transporting layer between the cathode and the emitting layer. In a case where the emitting layer has the first emitting layer and the second emitting layer, the organic EL device may have the electron transporting layer between the cathode and the second emitting layer in an exemplary arrangement.

In a case where the organic layer has the emitting layer, the organic EL device according to the exemplary embodiment preferably has the hole transporting layer between the anode and the emitting layer. In a case where the emitting layer has the first emitting layer and the second emitting layer, the organic EL device may have the hole transporting layer between the anode and the first emitting layer in another exemplary arrangement.

In the organic EL device according to the exemplary embodiment, a non-doped region, which does not contain a third compound that fluoresces, may also be provided on a side of the first emitting layer close to the anode.

The organic EL device according to the exemplary embodiment may have the first emitting layer, the second emitting layer, and the non-doped region disposed between the anode and the first emitting layer. The non-doped region may be, for instance, a non-doped organic layer provided on a side of the first emitting layer close to the anode and being in direct contact with the first emitting layer.

The non-doped organic layer does not contain a metal atom. A content ratio of each of all materials forming the non-doped organic layer with respect to the non-doped organic layer is preferably 10 mass % or more, more preferably more than 10 mass %. The non-doped organic layer preferably contains the compound according to the first exemplary embodiment. The non-doped organic layer also preferably consists of the compound according to the first exemplary embodiment.

The non-doped organic layer is, for instance, a second hole transporting layer or an electron blocking layer.

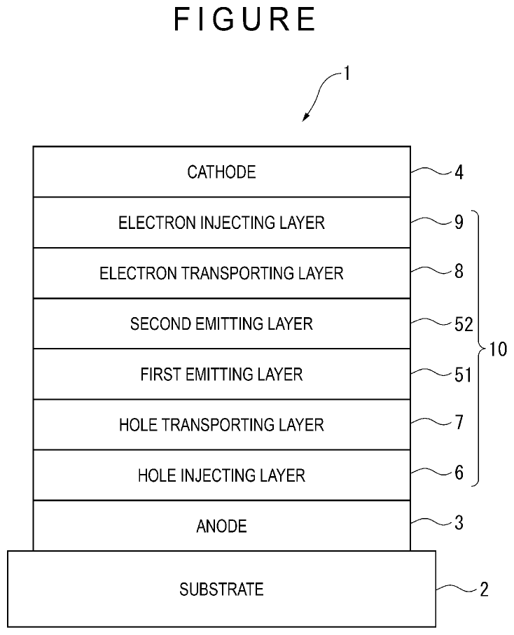

FIG. 1 schematically shows an exemplary arrangement of the organic EL device of the exemplary embodiment.

An organic EL device 1 includes a light-transmissive substrate 2, an anode 3, a cathode 4, and an organic layer 10 provided between the anode 3 and the cathode 4. The organic layer 10 includes a hole injecting layer 6, a hole transporting layer 7, a first emitting layer 51, a second emitting layer 52, an electron transporting layer 8, and an electron injecting layer 9, which are sequentially layered on the anode 3.

First Emitting Layer

In the organic EL device according to the exemplary embodiment, the first emitting layer preferably contains the first compound. The first compound is preferably a first host material.

In the organic EL device according to the exemplary embodiment, it is also preferable that the first emitting layer further contains a third compound that emits fluorescence. The first emitting layer also preferably contains the first compound as the first host material and the third compound as a first dopant material.

Herein, the “host material” refers to, for instance, a material that accounts for “50 mass % or more of the layer.” Accordingly, for instance, the first emitting layer contains 50 mass % or more of the first compound with respect to a total mass of the first emitting layer. Moreover, for instance, the “host material” may account for 60 mass % or more of the layer, 70 mass % or more of the layer, 80 mass % or more of the layer, 90 mass % or more of the layer, or 95 mass % or more of the layer.

First Compound

In the organic EL device according to the exemplary embodiment, the first compound is the compound according to the first exemplary embodiment.

In an organic EL device according to an exemplary embodiment, the first compound is a compound represented by the formula (1000B) and having at least one group represented by the formula (110).

In an organic EL device according to an exemplary embodiment, the first compound is a compound having at least one group represented by the formula (11) and a single benz[de]anthracene derivative skeleton represented by the formula (1000) in a molecule.

In an organic EL device according to an exemplary embodiment, the first compound is a compound having at least one group represented by the formula (110A) and having a single benz[de]anthracene derivative skeleton represented by the formula (1000A) in a molecule.

Second Emitting Layer

In the organic EL device according to the exemplary embodiment, the second emitting layer preferably contains a second compound. The second compound is preferably a second host material.

In the organic EL device according to the exemplary embodiment, it is also preferable that the second emitting layer further contains a fourth compound that emits fluorescence. The second emitting layer also preferably contains the second compound as the second host material and the fourth compound as a second dopant material.

When the first emitting layer contains the third compound and the the second emitting layer contains the fourth compound, the third compound and the fourth compound are mutually the same or different.

Second Compound

In the organic EL device according to the exemplary embodiment, the second compound is not particularly limited. In the organic EL device according to the exemplary embodiment, the second compound as the host material is exemplified by a heterocyclic compound and a fused aromatic compound. Examples of the fused aromatic compound include an anthracene derivative, a pyrene derivative, and a benzanthracene derivative.

The second compound is also preferably, for instance, a compound represented by a formula (2).

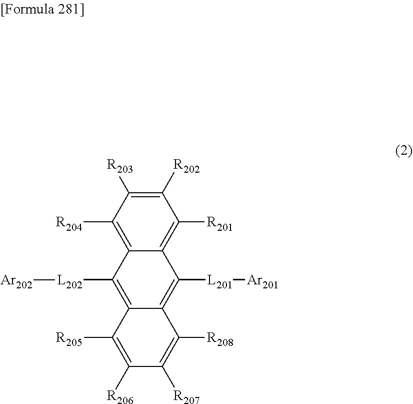

In the formula (2):
R₂₀₁ to R₂₀₈ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a nitro group, a substituted or unsubstituted aryl group having 6 to 50 ring carbon atoms, or a substituted or unsubstituted heterocyclic group having 5 to 50 ring atoms;
L₂₀₁ and L₂₀₂ are each independently a single bond, a substituted or unsubstituted arylene group having 6 to 50 ring carbon atoms, or a substituted or unsubstituted divalent heterocyclic group having 5 to 50 ring atoms;
Ar₂₀₁ and Ar₂₀₂ are each independently a substituted or unsubstituted aryl group having 6 to 50 ring carbon atoms, or a substituted or unsubstituted heterocyclic group having 5 to 50 ring atoms.

In the second compound according to the exemplary embodiment, R₉₀₁, R₉₀₂, R₉₀₃, R₉₀₄, R₉₀₅, R₉₀₅, R₉₀₇, R₈₀₁, and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₅ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

In the organic EL device according to the exemplary embodiment, it is preferable that R₂₀₁ to R₂₀₅ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, or a nitro group;
L₂₀₁ and L₂₀₂ are each independently a single bond, a substituted or unsubstituted arylene group having 6 to 50 ring carbon atoms, or a substituted or unsubstituted divalent heterocyclic group having 5 to 50 ring atoms;
Ar₂₀₁ and Ar₂₀₂ are each independently a substituted or unsubstituted aryl group having 6 to 50 ring carbon atoms, or a substituted or unsubstituted heterocyclic group having 5 to 50 ring atoms.

In the second compound, R₂₀₁ to R₂₀₅ also preferably each independently a hydrogen atom, a substituted or unsubstituted alkyl group having 1 to 50 carbon atoms, a substituted or unsubstituted cycloalkyl having 3 to 50 ring carbon atoms, or a group represented by —Si(R₉₀₁)(R₉₀₂)(R₉₀₃).

In the second compound, R₂₀₁ to R₂₀₈ are also preferably each a hydrogen atom.

In the organic EL device according to the exemplary embodiment, it is preferable that L₂₀₁ and L₂₀₂ are each independently a single bond or a substituted or unsubstituted arylene group having 6 to 50 ring carbon atoms and Ar₂₀₁ and Ar₂₀₂ are each independently a substituted or unsubstituted aryl group having 6 to 50 ring carbon atoms.

In the organic EL device according to the exemplary embodiment, Ar₂₀₁ and Ar₂₀₂ are preferably each independently phenyl group, a naphthyl group, phenanthryl group, a biphenyl group, a terphenyl group, a diphenylfluorenyl group, a dimethylfluorenyl group, a benzodiphenylfluorenyl group, a benzodimethylfluorenyl group, dibenzofuranyl group, a dibenzothienyl group, a benzoxanthenyl group, a naphthobenzofuranyl group, or a naphthobenzothienyl group.

In the organic EL device according to the exemplary embodiment, the second compound represented by the formula (2) is preferably a compound represented by a formula (201), (202), (203), (204), (205), (206), (207), (208) or (209) below.

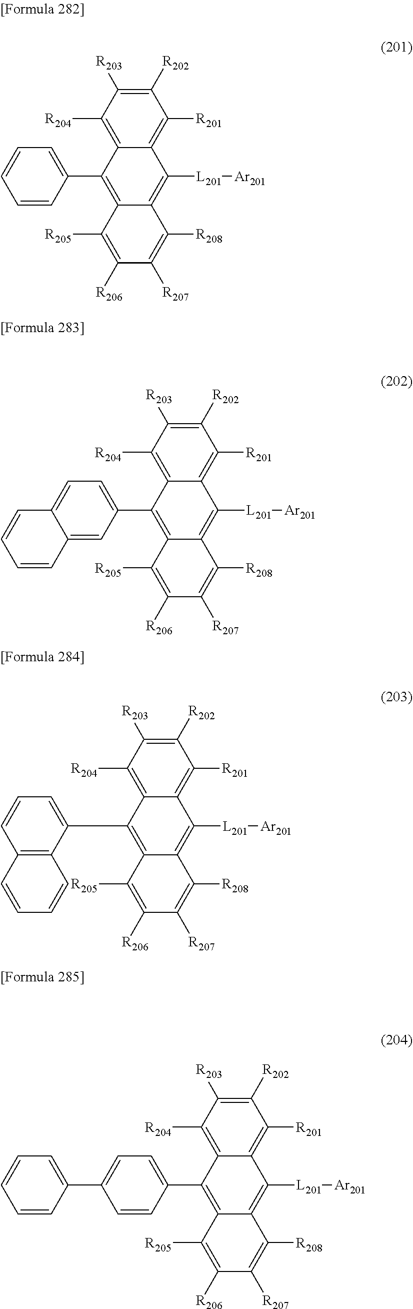

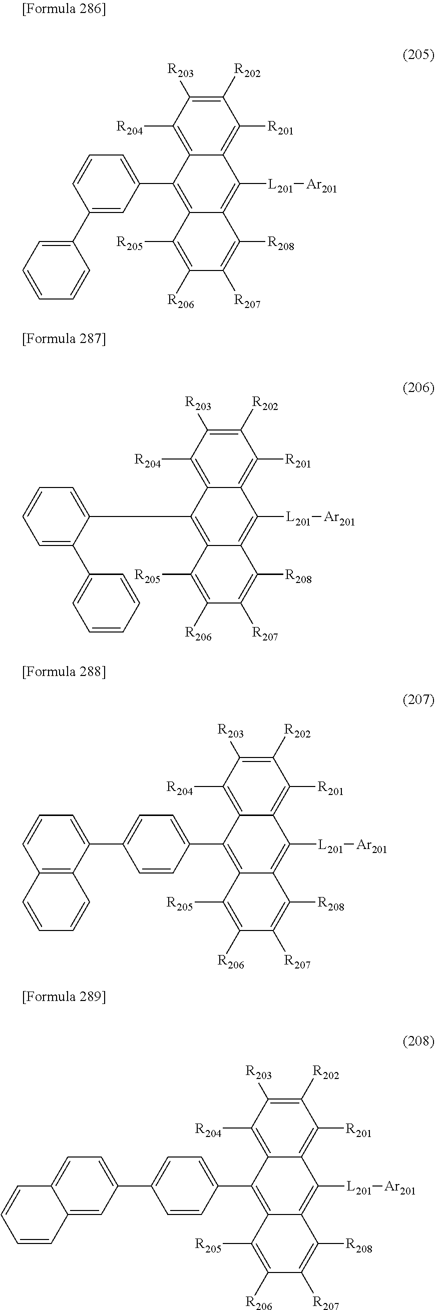

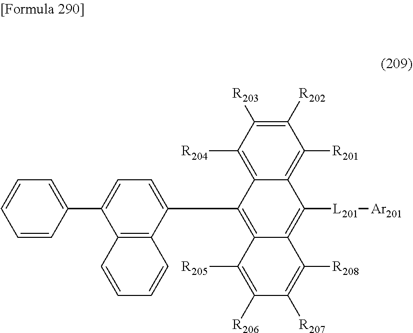

In the formulae (201) to (209), L₂₀₁ and Ar₂₀₁ represent the same as L₂₀₁ and Ar₂₀₁ in the formula (2), and R₂₀₁ to R₂₀₈ each independently represent the same as R₂₀₁ to R₂₀₈ in the formula (2).

The second compound represented by the formula (2) is also preferably a compound represented by a formula (221), (222), (223), (224), (225), (226), (227), (228) or (229) below.

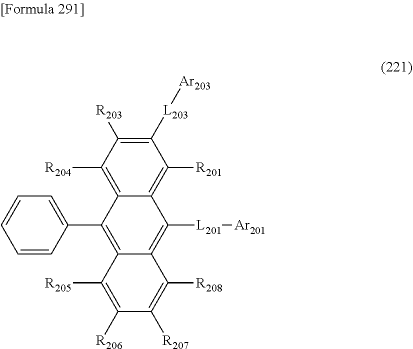

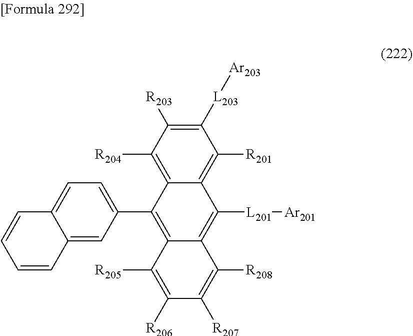

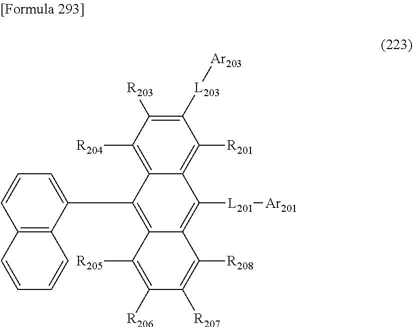

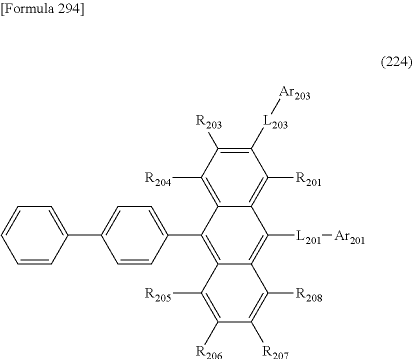

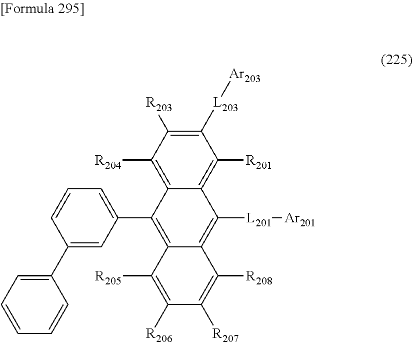

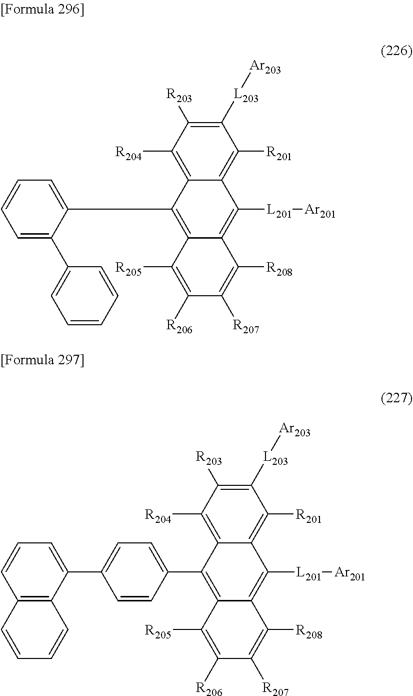

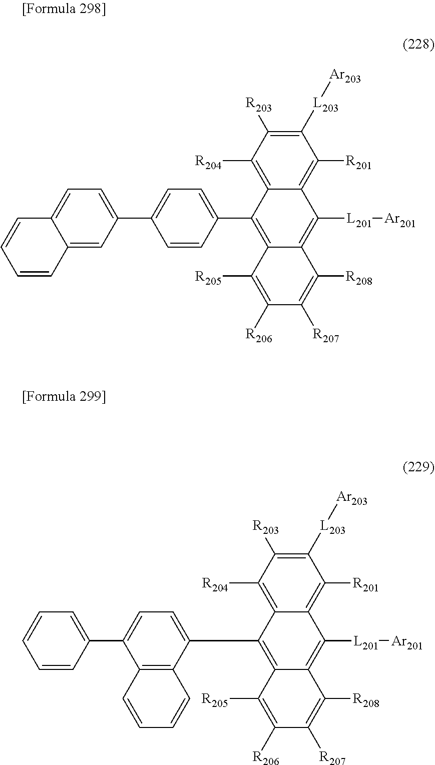

In the formulae (221), (222), (223), (224), (225), (226), (227), (228) and (229):
R₂₀₁ and R₂₀₃ to R₂₀₅ each independently represent the same as R₂₀₁ and R₂₀₃ to R₂₀₅ in the formula (2);
L₂₀₁ and Ar₂₀₁ respectively represent the same as L₂₀₁ and Ar₂₀₁ in the formula (2);
L₂₀₃ represents the same as L₂₀₁ in the formula (2);
L₂₀₃ and L₂₀₁ are mutually the same or different;
Ar₂₀₃ represents the same as Ar₂₀₁ in the formula (2); and
Ar₂₀₃ and Ar₂₀₁ are mutually the same or different.

The second compound represented by the formula (2) is also preferably a compound represented by a formula (241), (242), (243), (244), (245), (246), (247), (248) or (249) below.

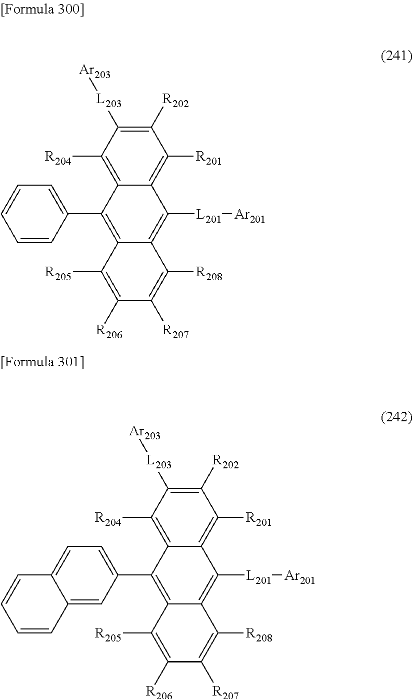

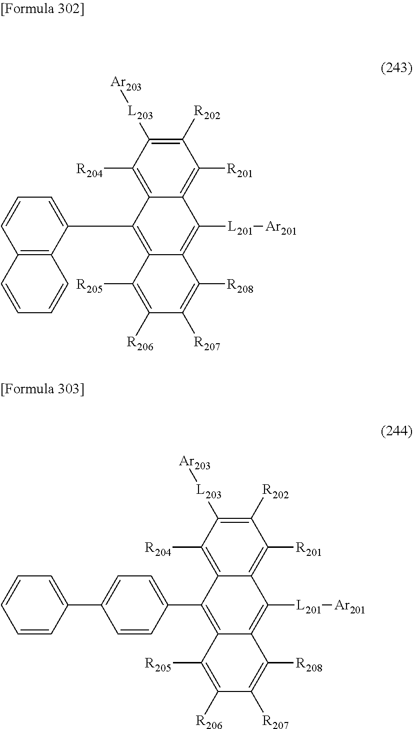

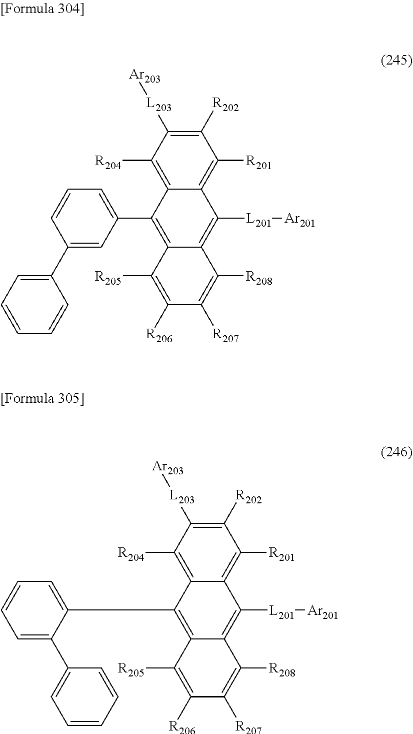

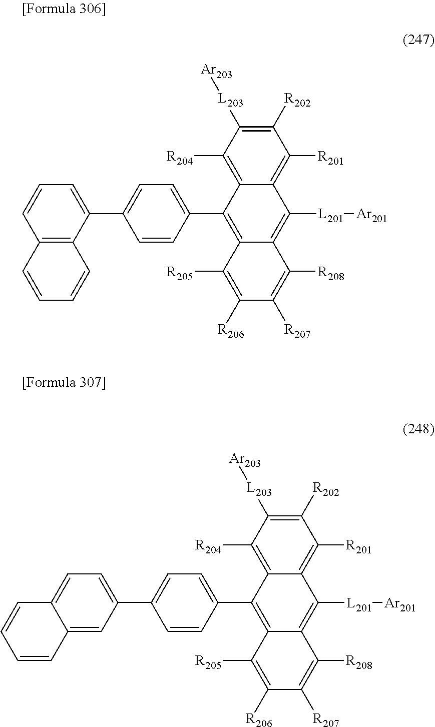

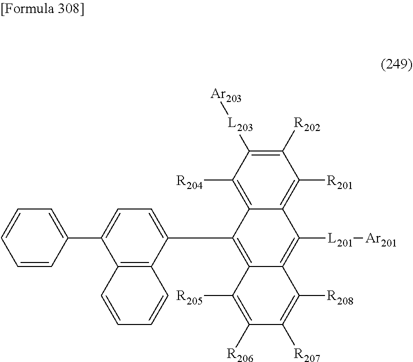

In the formulae (241), (242), (243), (244), (245), (246), (247), (248) and (249):
R₂₀₁, R₂₀₂ and R₂₀₄ to R₂₀₈ each independently represent the same as R₂₀₁, R₂₀₂ and R₂₀₄ to R₂₀₈ in the formula (2);
L₂₀₁ and Ar₂₀₁ respectively represent the same as L₂₀₁ and Ar₂₀₁ in the formula (2);
L₂₀₃ represents the same as L₂₀₁ in the formula (2);
L₂₀₃ and L₂₀₁ are mutually the same or different;
Ar₂₀₃ represents the same as Ar₂₀₁ in the formula (2); and
Ar₂₀₃ and Ar₂₀₁ are mutually the same or different.

R₂₀₁ to R₂₀₈ in the second compound represented by the formula (2) are preferably each independently a hydrogen atom, a substituted or unsubstituted alkyl group having 1 to 50 carbon atoms, a substituted or unsubstituted cycloalkyl group having 3 to 50 ring carbon atoms, or a group represented by —Si(R₉₀₁)(R₉₀₂)(R₉₀₃).

L₁₀₁ is preferably a single bond, or an unsubstituted arylene group having 6 to 22 ring carbon atoms.

Ar₁₀₁ is preferably a substituted or unsubstituted aryl group having 6 to 22 ring carbon atoms.

In the organic EL device according to the exemplary embodiment, R₂₀₁ to R₂₀₈ that are substituents on an anthracene skeleton in the second compound represented by the formula (2) are preferably hydrogen atoms in terms of preventing inhibition of intermolecular interaction to inhibit a decrease in electron mobility. However, R₂₀₁ to R₂₀₈ may be a substituted or unsubstituted aryl group having 6 to 50 ring carbon atoms or a substituted or unsubstituted heterocyclic group having 5 to 50 ring atoms.

Assuming that R₂₀₁ to R₂₀₈ each are a bulky substituent such as an alkyl group and a cycloalkyl group, intermolecular interaction may be inhibited to decrease the electron mobility of the second compound relative to that of the first compound, so that a relationship of μH2>μH1 shown by a numerical formula below (Numerical Formula 3) may not be satisfied. When the second compound is used in the second emitting layer, it can be expected that satisfying the relationship of μH2>μH1 inhibits a decrease in a recombination ability between holes and electrons in the first emitting layer and a decrease in a luminous efficiency. It should be noted that substituents, namely, a haloalkyl group, alkenyl group, alkynyl group, group represented by —Si(R₉₀₁)(R₉₀₂)(R₉₀₃), group represented by —O—(R₉₀₄), group represented by —S—(R₉₀₅), group represented by —N(R₉₀₆)(R₉₀₇), aralkyl group, group represented by —C(═O)R₈₀₁, group represented by —COOR₈₀₂, halogen atom, cyano group, and nitro group are likely to be bulky, and an alkyl group and cycloalkyl group are likely to be further bulky.

In the second compound represented by the formula (2), R₂₀₁ to R₂₀₅ being the substituents on the anthracene skeleton are each preferably not a bulky substituent and preferably not an alkyl group and cycloalkyl group. More preferably, R₂₀₁ to R₂₀₈ are not an alkyl group, cycloalkyl group, haloalkyl group, alkenyl group, alkynyl group, group represented by —Si(R₉₀₁)(R₉₀₂)(R₉₀₃), group represented by —O—(R₉₀₄), group represented by —S—(R₉₀₅), group represented by —N(R₉₀₆)(R₉₀₇), aralkyl group, group represented by C(═O)R₈₀₁, group represented by —COOR₈₀₂, halogen atom, cyano group, and nitro group.

In the second compound represented by the formula (2) in the organic EL device according to the exemplary embodiment, R₂₀₁ to R₂₀₈ also preferably each independently a hydrogen atom, a substituted or unsubstituted alkyl group having 1 to 50 carbon atoms, a substituted or unsubstituted cycloalkyl having 3 to 50 ring carbon atoms, or a group represented by —Si(R₉₀₁)(R₉₀₂)(R₉₀₃).

In the second compound represented by the formula (2) in the organic EL device according to the exemplary embodiment, R₂₀₁ to R₂₀₅ are preferably each a hydrogen atom.

In the second compound, it is also preferable that examples of a substituent for a “substituted or unsubstituted group” on R₂₀₁ to R₂₀₈ do not include the above-described substituent that is likely to be bulky, especially a substituted or unsubstituted alkyl group and a substituted or unsubstituted cycloalkyl group. Since the examples of the substituent for a “substituted or unsubstituted” group on R₂₀₁ to R₂₀₅ do not include a substituted or unsubstituted alkyl group and a substituted or unsubstituted cycloalkyl group, inhibition of intermolecular interaction to be caused by presence of a bulky substituent such as an alkyl group and a cycloalkyl group can be prevented, thereby preventing a decrease in the electron mobility. Moreover, when the second compound described above is used in the second emitting layer, a decrease in a recombination ability between holes and electrons in the first emitting layer and a decrease in the luminous efficiency can be inhibited.

It is more preferable that R₂₀₁ to R₂₀₈ being the substituents on the anthracene skeleton are not bulky substituents, and R₂₀₁ to R₂₀₅ as substituents are unsubstituted. Assuming that R₂₀₁ to R₂₀₈ being the substituents on the anthracene skeleton are not bulky substituents and substituents are bonded to R₂₀₁ to R₂₀₈ which are the not-bulky substituents, the substituents bonded to R₂₀₁ to R₂₀₅ are preferably not the bulky substituents; the substituents bonded to R₂₀₁ to R₂₀₈ serving as substituents are preferably not an alkyl group and cycloalkyl group, more preferably not an alkyl group, cycloalkyl group, haloalkyl group, alkenyl group, alkynyl group, group represented by —Si(R₉₀₁)(R₉₀₂)(R₉₀₃), group represented by —O—(R₉₀₄), group represented by —S—(R₉₀₅), group represented by —N(R₉₀₆)(R₉₀₇), aralkyl group, group represented by —C(═O)R₈₀₁, group represented by —COOR₈₀₂, halogen atom, cyano group, and nitro group.

In the first compound and the second compound, it is also preferable that all of the “substituted or unsubstituted” groups are “unsubstituted” groups.

In the organic EL device according to the exemplary embodiment, for instance, Ar₂₀₁ in the second compound represented by the formula (2) is a substituted or unsubstituted dibenzofuranyl group.

In the organic EL device according to the exemplary embodiment, for instance, Ar₂₀₁ in the second compound represented by the formula (2) is an unsubstituted dibenzofuranyl group.

In the organic EL device according to the exemplary embodiment, for instance, the second compound represented by the formula (2) has at least one hydrogen atom, the hydrogen atom including at least one deuterium atom.

In the organic EL device according to the exemplary embodiment, for instance, L₂₀₁ in the second compound represented by the formula (2) is one of TEMP-63 to TEMP-68.

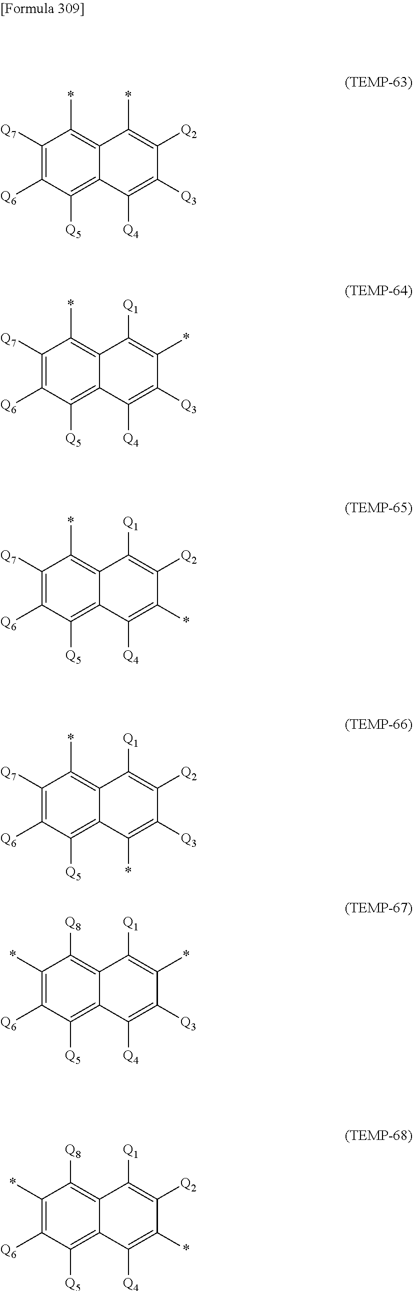

In the formulae (TEMP-63) to (TEMP-68), * represents a bonding position.

In the second compound represented by the formula (2) in the organic EL device according to the exemplary embodiment, Ar₂₀₁ is at least one group selected from the group consisting of a substituted or unsubstituted anthryl group, benzanthryl group, phenanthryl group, benzophenanthryl group, phenalenyl group, pyrenyl group, chrysenyl group, benzochrysenyl group, triphenylenyl group, benzotriphenylenyl group, tetracenyl group, pentacenyl group, fluoranthenyl group, benzofluoranthenyl group, and perylenyl group.

In the organic EL device according to the exemplary embodiment, for instance, Ar₂₀₁ in the second compound represented by the formula (2) is a substituted or unsubstituted fluorenyl group.

In the organic EL device according to the exemplary embodiment, for instance, Ar₂₀₁ in the second compound represented by the formula (2) is a substituted or unsubstituted xanthenyl group.

In the organic EL device according to the exemplary embodiment, for instance, Ar₂₀₁ in the second compound represented by the formula (2) is a benzoxanthenyl group.

Manufacturing Method of Second Compound

The second compound can be manufactured by a known method. The second compound can also be manufactured based on a known method through a known alternative reaction using a known material(s) tailored for the target compound.

### Specific Examples of Second Compound

Examples of the second compound include compounds below. It should however be noted that the invention is not limited to the specific examples of the second compound.

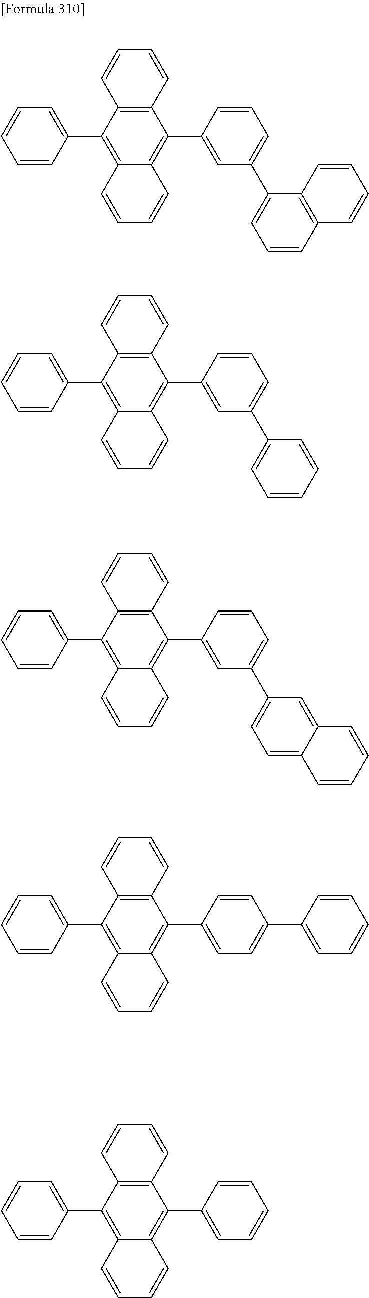

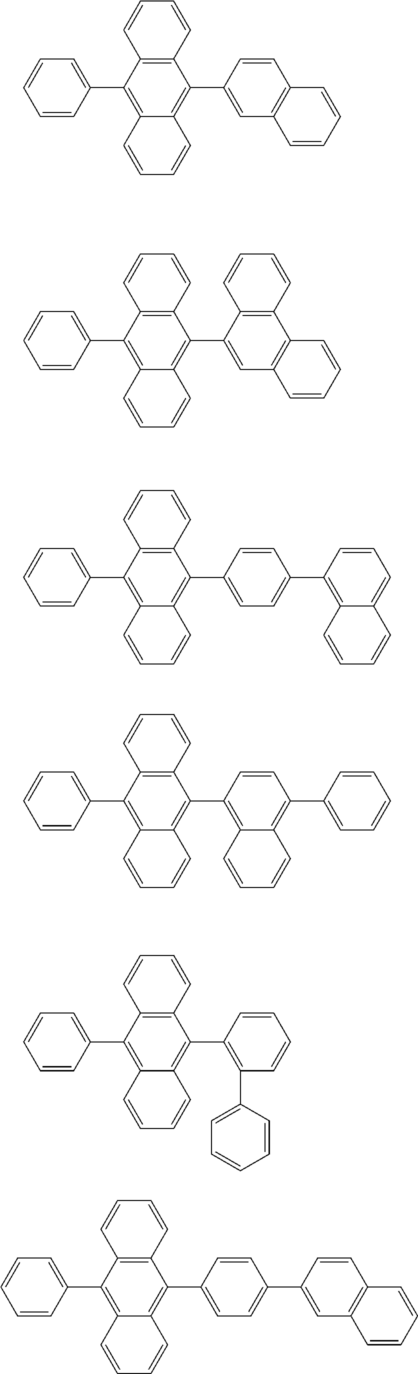

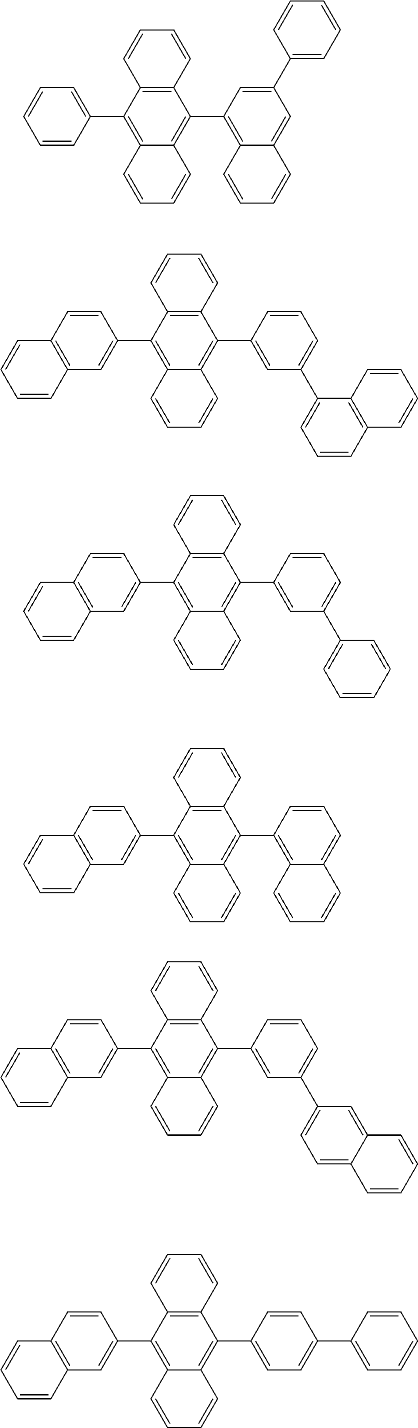

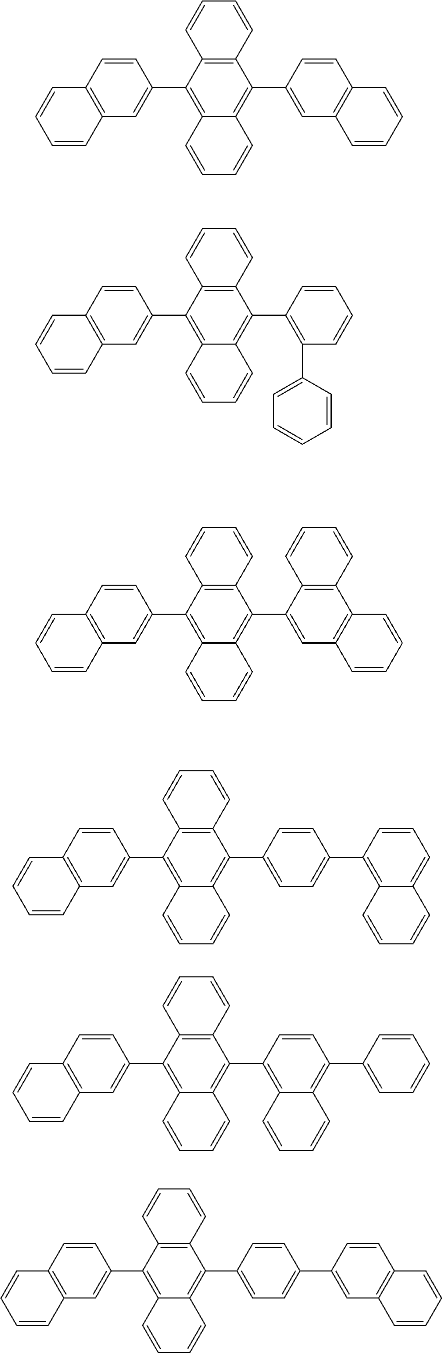

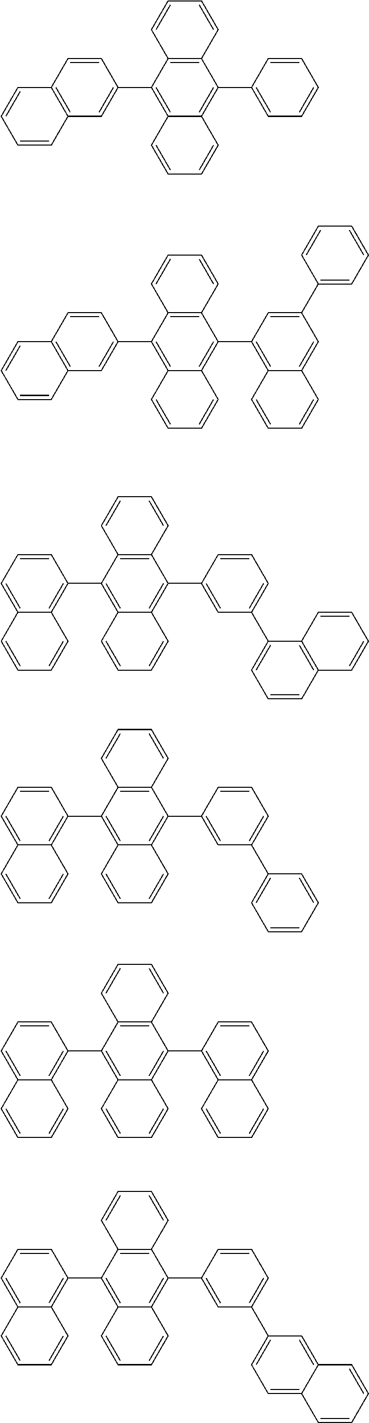

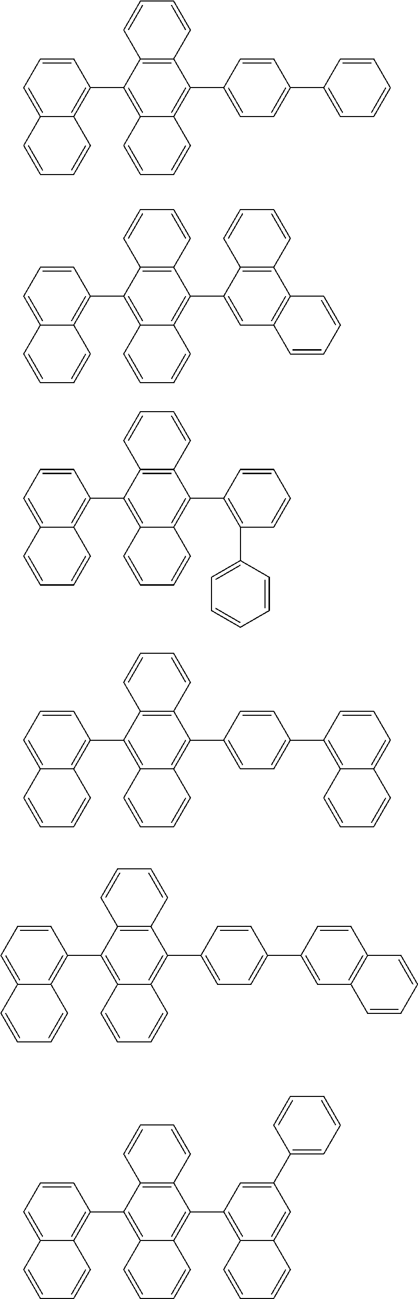

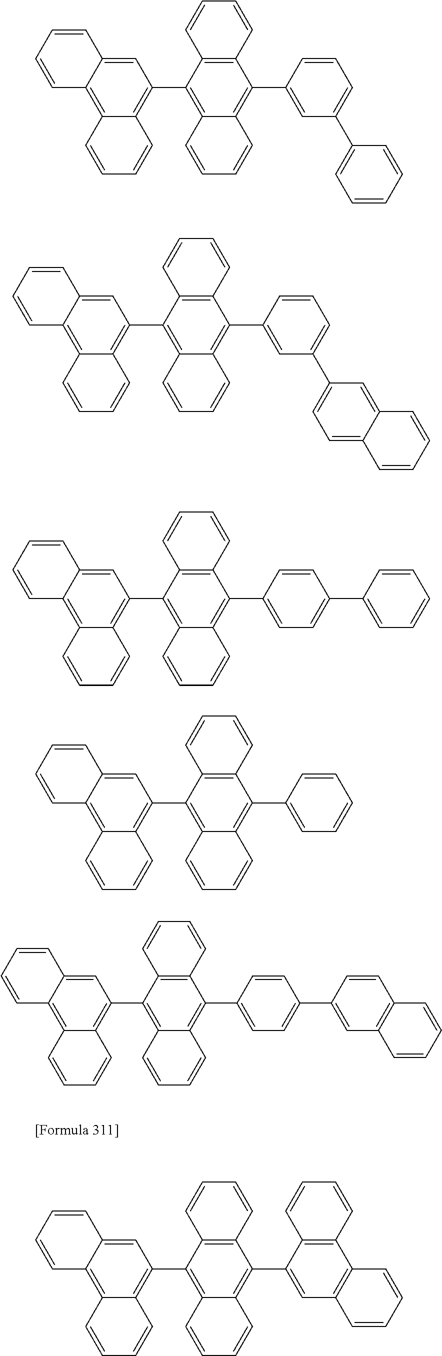

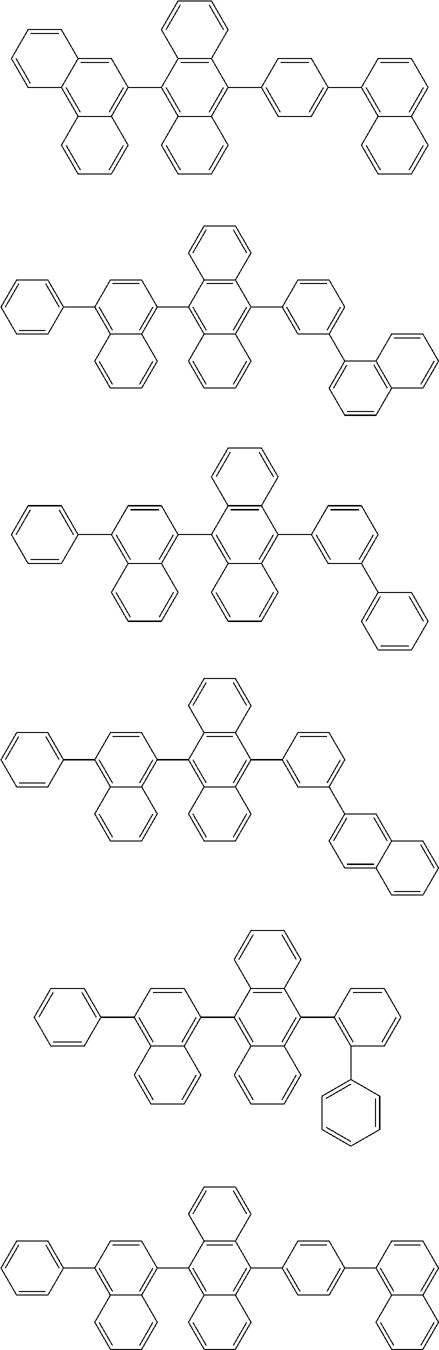

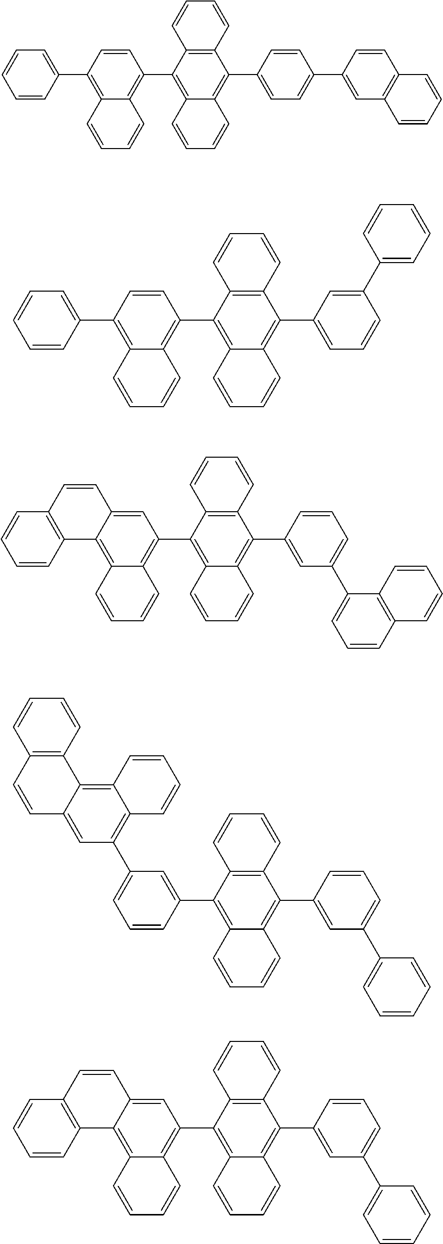

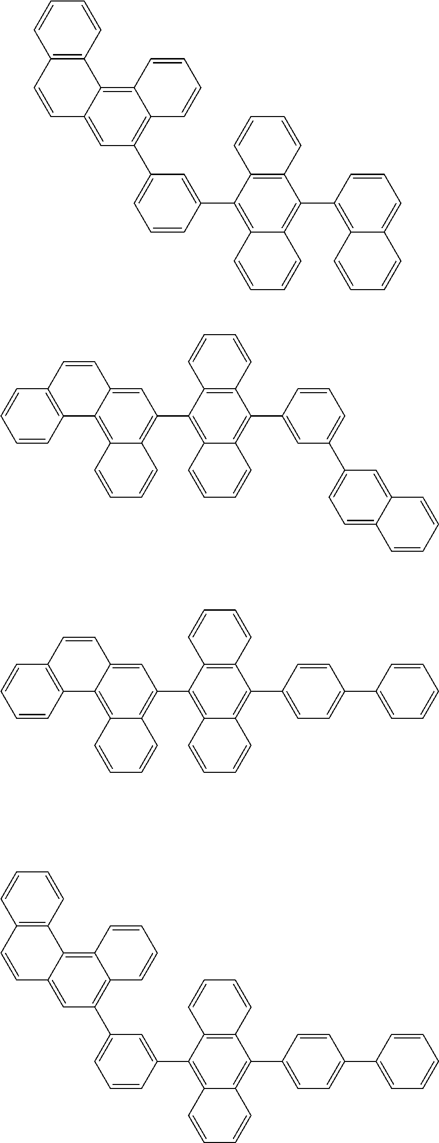

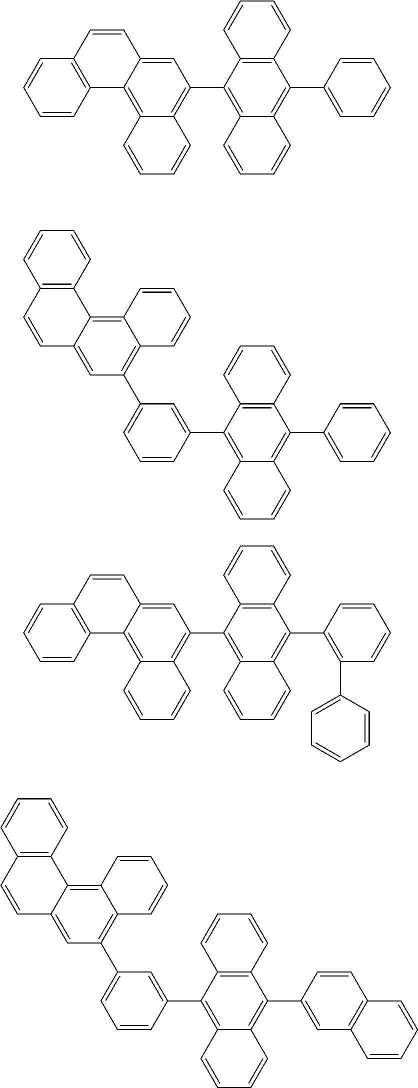

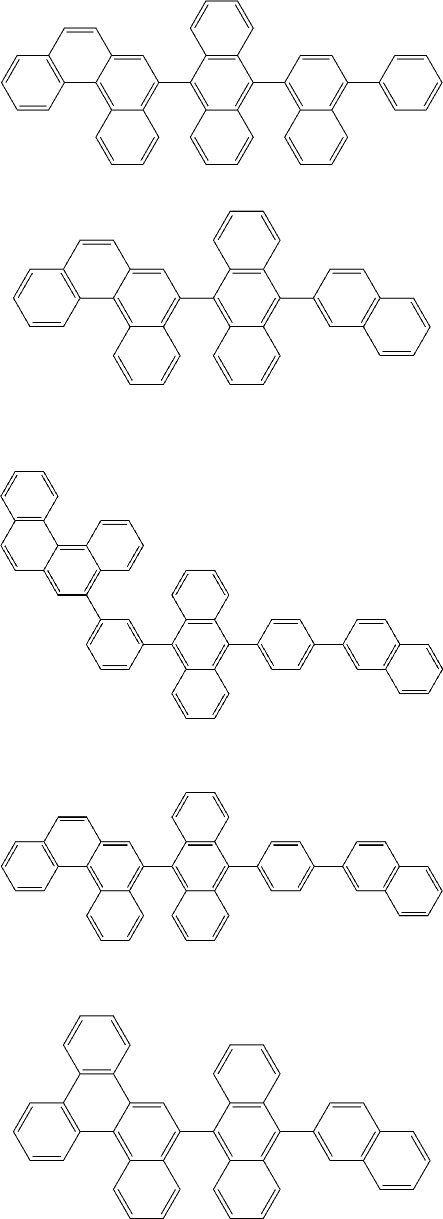

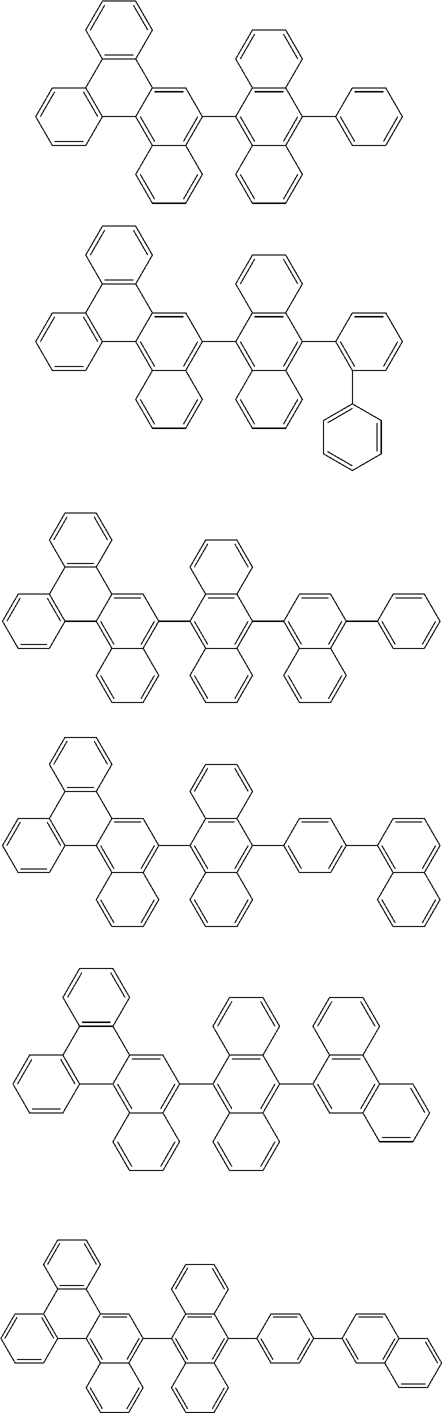

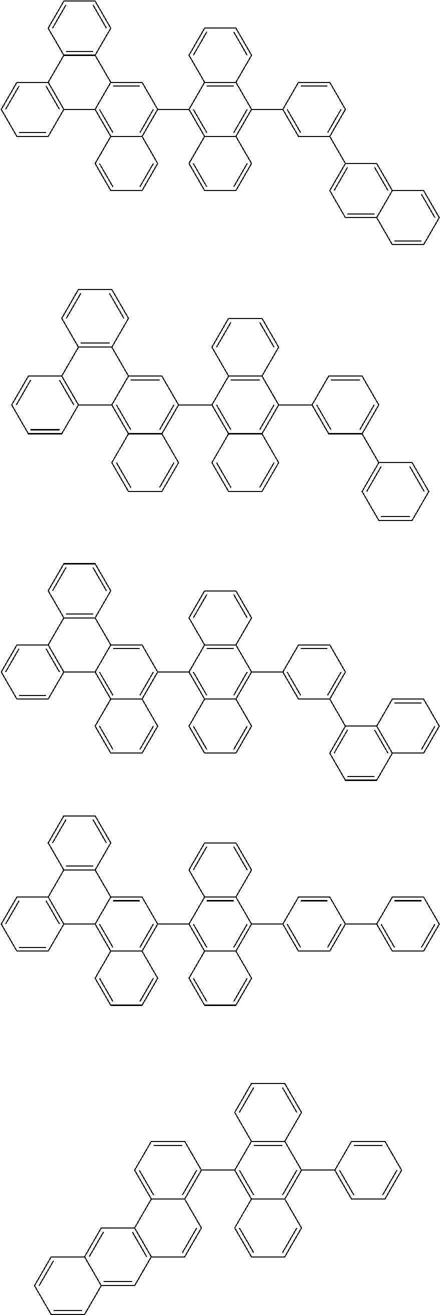

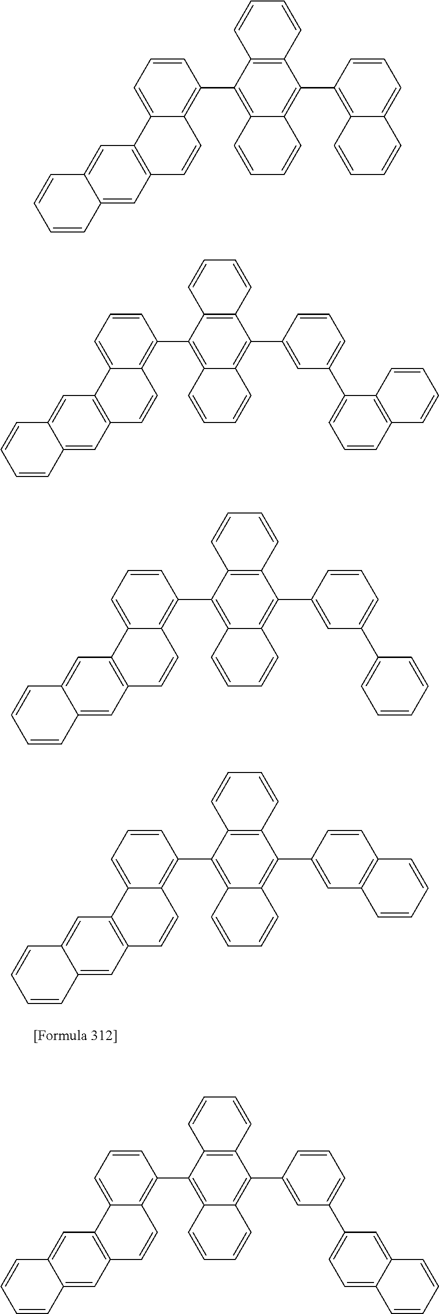

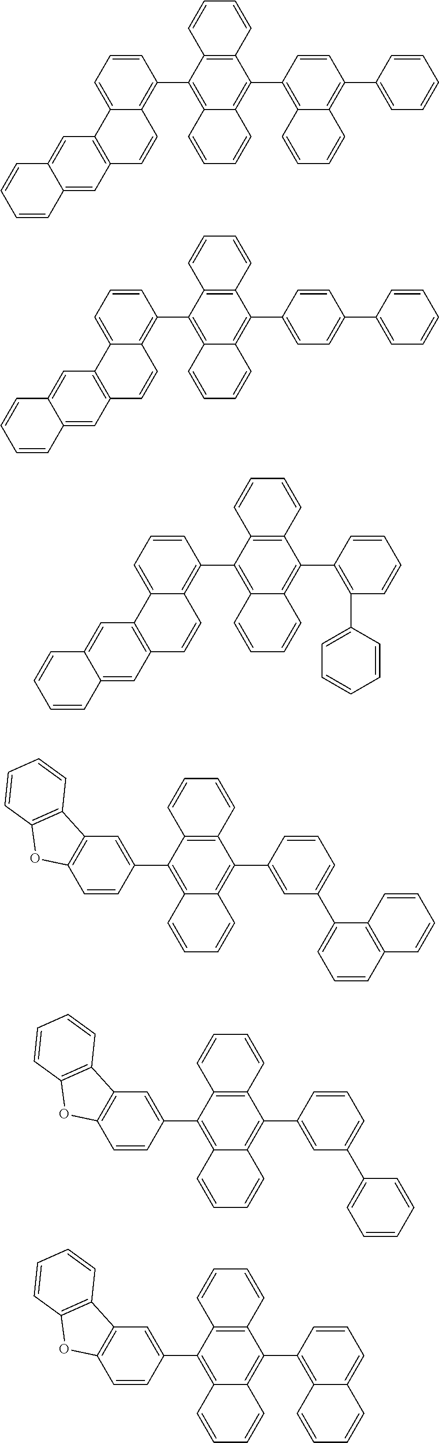

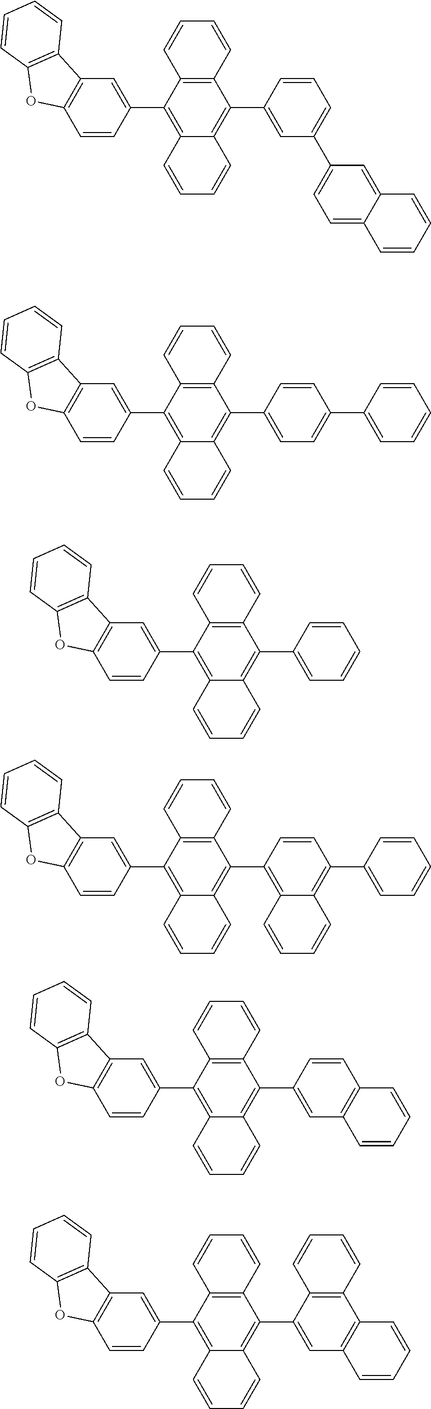

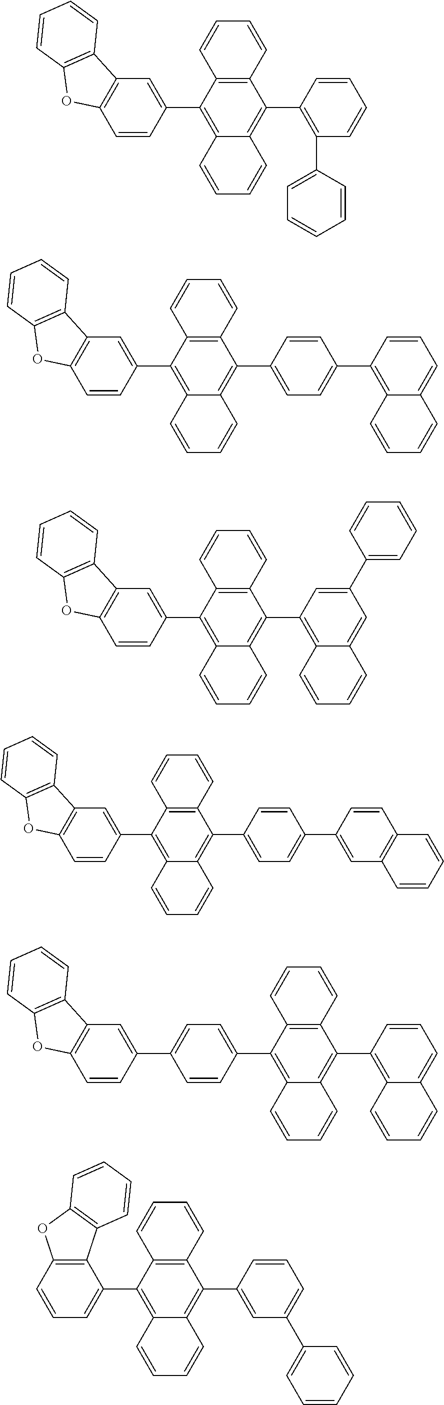 [SMILES: c1ccc(-c2ccccc2-c2c3ccccc3c(-c3ccc4oc5ccccc5c4c3)c3ccccc23)cc1 ; c1ccc2c(-c3ccc(-c4c5ccccc5c(-c5ccc6oc7ccccc7c6c5)c5ccccc45)cc3)cccc2c1 ; c1ccc(-c2cc(-c3c4ccccc4c(-c4ccc5oc6ccccc6c5c4)c4ccccc34)c3ccccc3c2)cc1 ; c1ccc2cc(-c3ccc(-c4c5ccccc5c(-c5ccc6oc7ccccc7c6c5)c5ccccc45)cc3)ccc2c1 ; c1ccc2c(-c3c4ccccc4c(-c4ccc(-c5ccc6oc7ccccc7c6c5)cc4)c4ccccc34)cccc2c1 ; c1ccc(-c2cccc(-c3c4ccccc4c(-c4cccc5oc6ccccc6c45)c4ccccc34)c2)cc1]

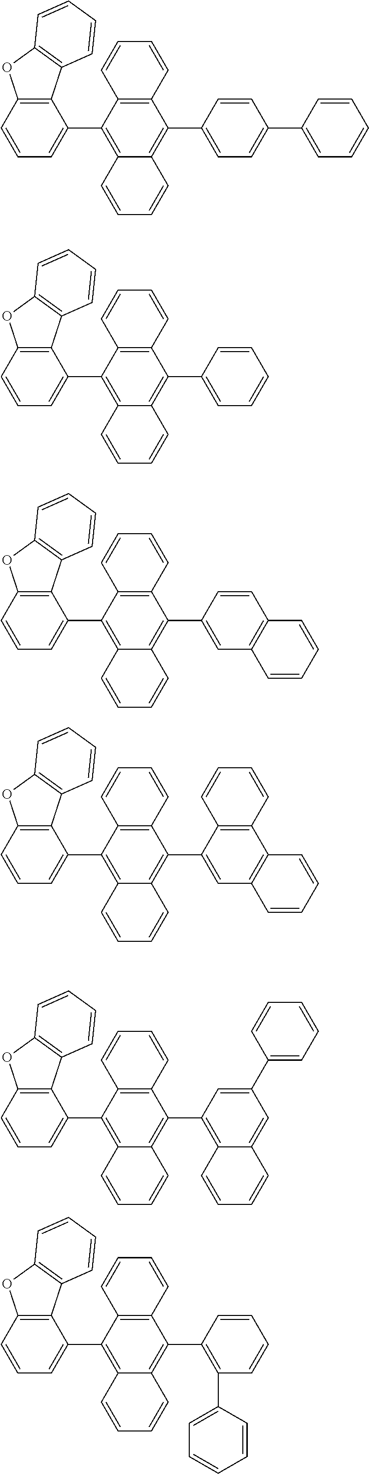

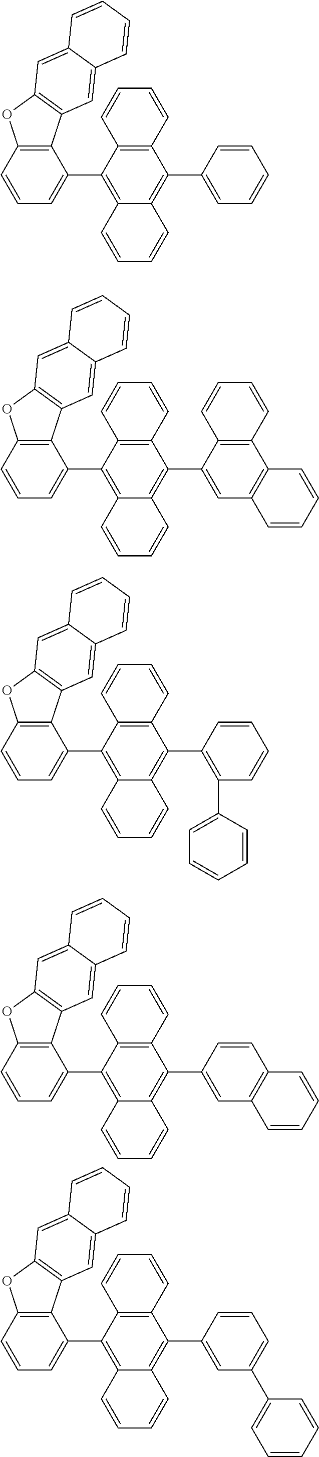

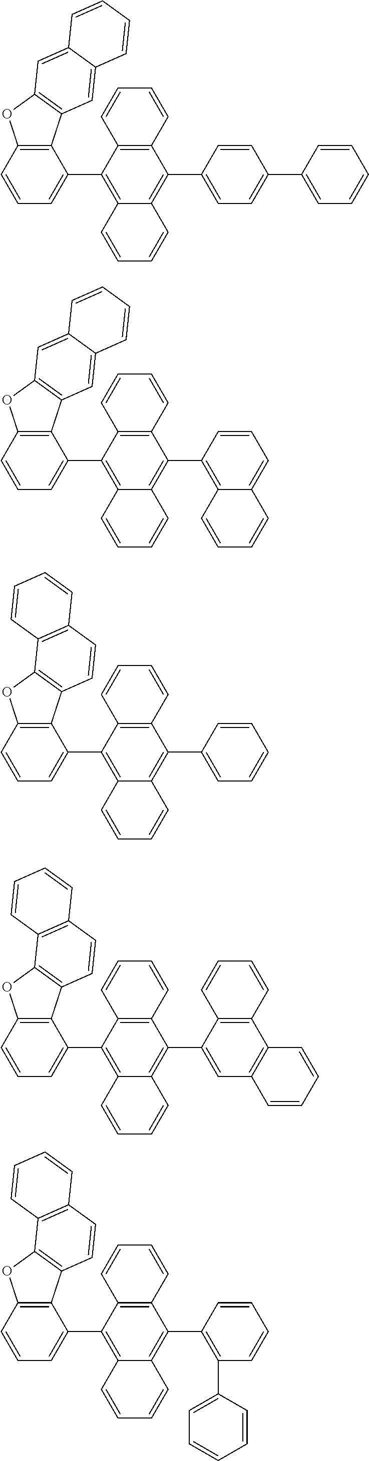

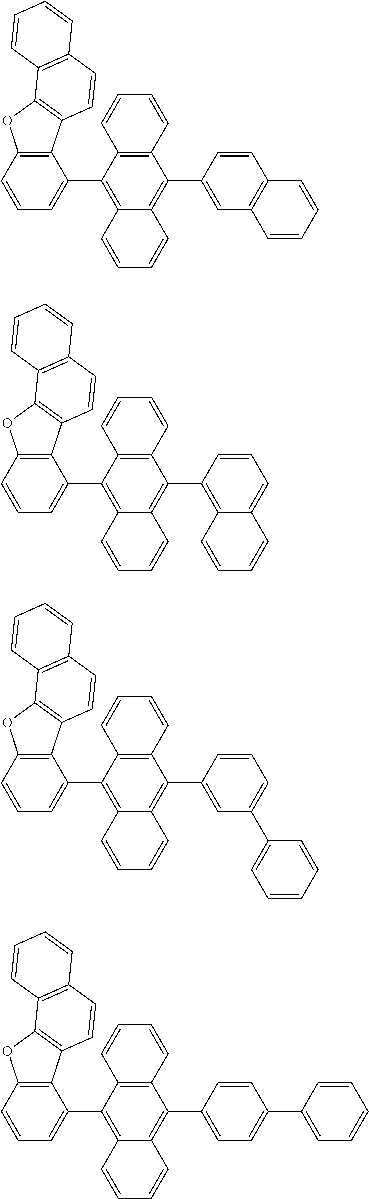 [SMILES: c1ccc2cc(-c3c4ccccc4c(-c4cccc5oc6c7ccccc7ccc6c45)c4ccccc34)ccc2c1 ; c1ccc2c(-c3c4ccccc4c(-c4cccc5oc6c7ccccc7ccc6c45)c4ccccc34)cccc2c1 ; c1ccc(-c2cccc(-c3c4ccccc4c(-c4cccc5oc6c7ccccc7ccc6c45)c4ccccc34)c2)cc1 ; c1ccc(-c2ccc(-c3c4ccccc4c(-c4cccc5oc6c7ccccc7ccc6c45)c4ccccc34)cc2)cc1]

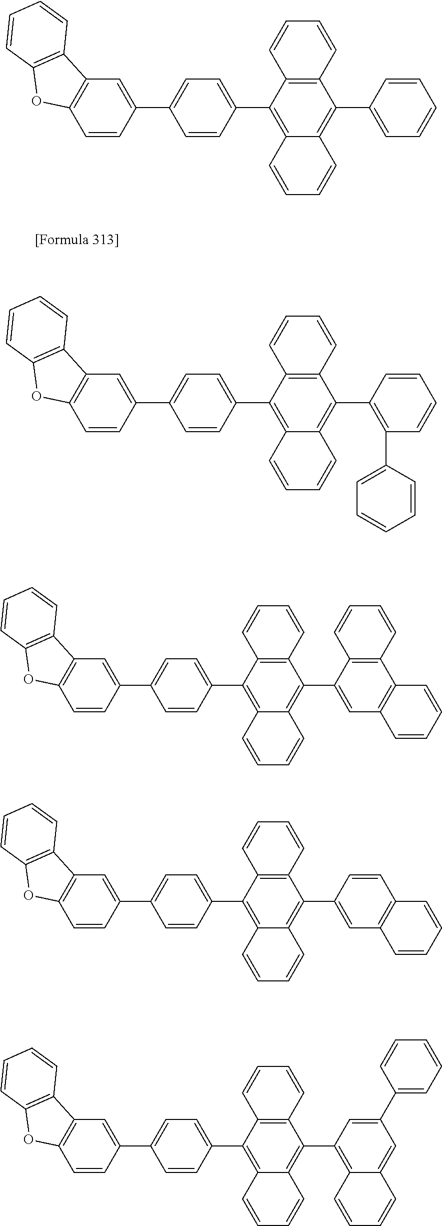 [SMILES: c1ccc(-c2c3ccccc3c(-c3ccc(-c4ccc5oc6ccccc6c5c4)cc3)c3ccccc23)cc1 ; c1ccc(-c2ccccc2-c2c3ccccc3c(-c3ccc(-c4ccc5oc6ccccc6c5c4)cc3)c3ccccc23)cc1 ; c1ccc2c(c1)cc(-c1c3ccccc3c(-c3ccc(-c4ccc5oc6ccccc6c5c4)cc3)c3ccccc13)c1ccccc12 ; c1ccc2cc(-c3c4ccccc4c(-c4ccc(-c5ccc6oc7ccccc7c6c5)cc4)c4ccccc34)ccc2c1 ; c1ccc(-c2cc(-c3c4ccccc4c(-c4ccc(-c5ccc6oc7ccccc7c6c5)cc4)c4ccccc34)c3ccccc3c2)cc1]

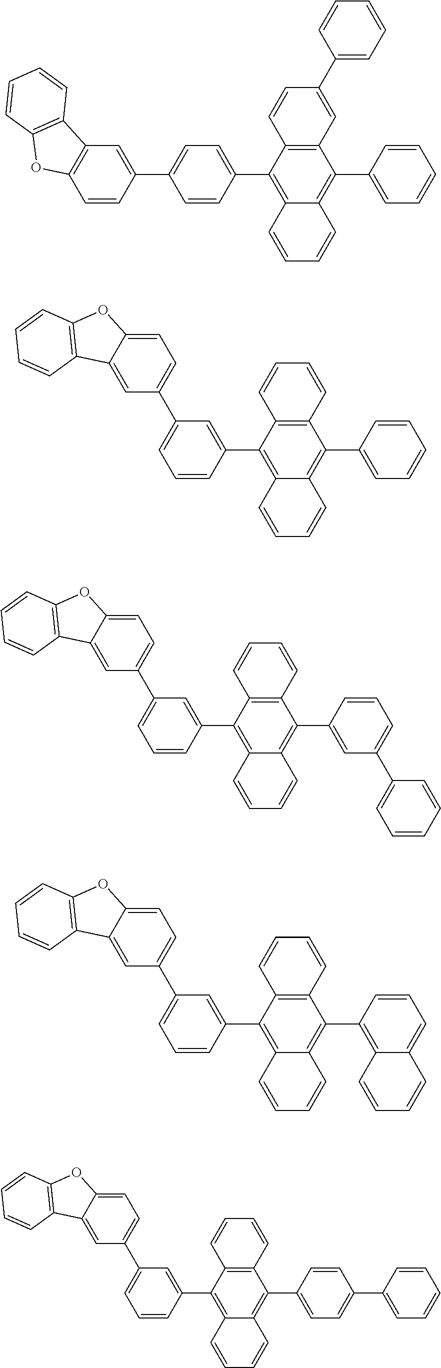 [SMILES: c1ccc(-c2ccc3c(-c4ccc(-c5ccc6oc7ccccc7c6c5)cc4)c4ccccc4c(-c4ccccc4)c3c2)cc1 ; c1ccc(-c2c3ccccc3c(-c3cccc(-c4ccc5oc6ccccc6c5c4)c3)c3ccccc23)cc1 ; c1ccc(-c2cccc(-c3c4ccccc4c(-c4cccc(-c5ccc6oc7ccccc7c6c5)c4)c4ccccc34)c2)cc1 ; c1cc(-c2ccc3oc4ccccc4c3c2)cc(-c2c3ccccc3c(-c3cccc4ccccc34)c3ccccc23)c1 ; c1ccc(-c2ccc(-c3c4ccccc4c(-c4cccc(-c5ccc6oc7ccccc7c6c5)c4)c4ccccc34)cc2)cc1]

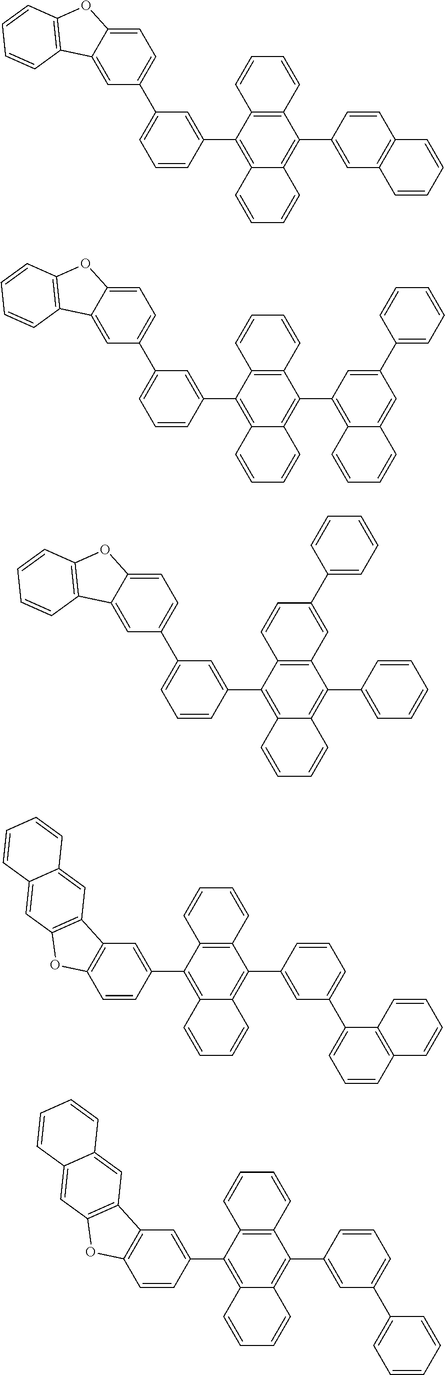

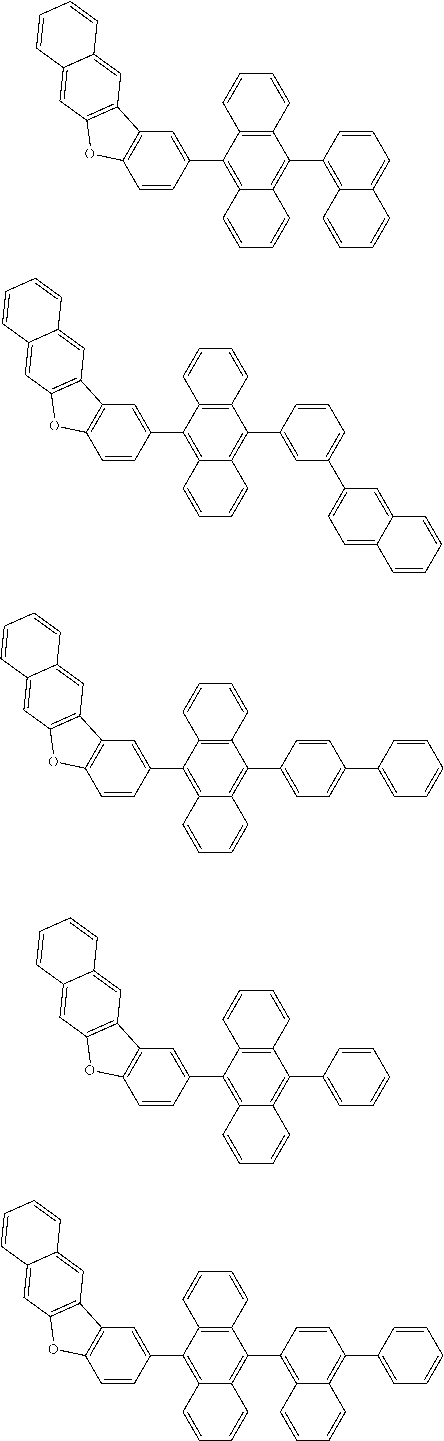 [SMILES: c1ccc2cc3c(cc2c1)oc1ccc(-c2c4ccccc4c(-c4cccc5ccccc45)c4ccccc24)cc13 ; c1cc(-c2ccc3ccccc3c2)cc(-c2c3ccccc3c(-c3ccc4oc5cc6ccccc6cc5c4c3)c3ccccc23)c1 ; c1ccc(-c2ccc(-c3c4ccccc4c(-c4ccc5oc6cc7ccccc7cc6c5c4)c4ccccc34)cc2)cc1 ; c1ccc(-c2c3ccccc3c(-c3ccc4oc5cc6ccccc6cc5c4c3)c3ccccc23)cc1 ; c1ccc(-c2ccc(-c3c4ccccc4c(-c4ccc5oc6cc7ccccc7cc6c5c4)c4ccccc34)c3ccccc23)cc1]

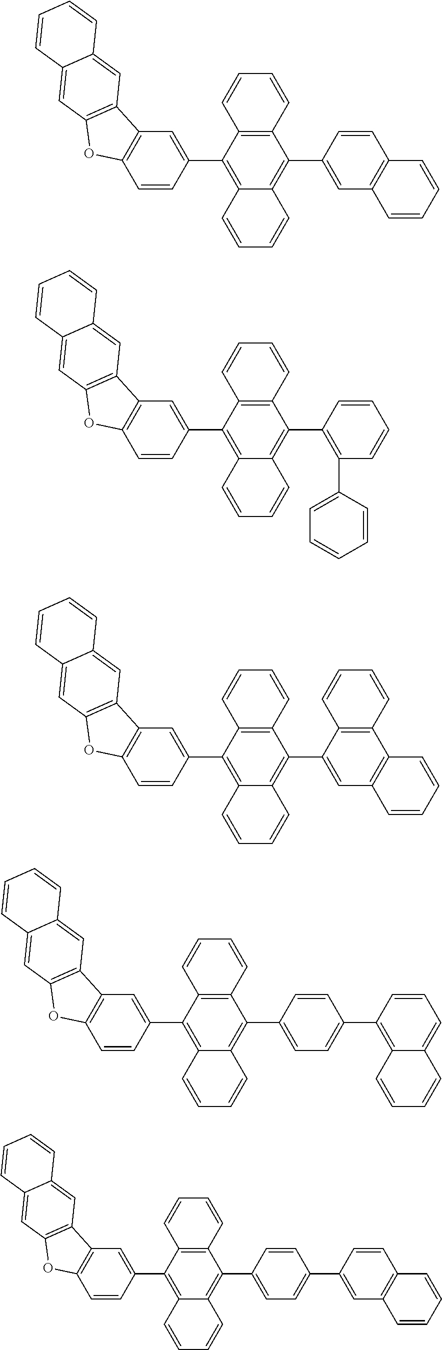 [SMILES: c1ccc2cc(-c3c4ccccc4c(-c4ccc5oc6cc7ccccc7cc6c5c4)c4ccccc34)ccc2c1 ; c1ccc(-c2ccccc2-c2c3ccccc3c(-c3ccc4oc5cc6ccccc6cc5c4c3)c3ccccc23)cc1 ; c1ccc2cc3c(cc2c1)oc1ccc(-c2c4ccccc4c(-c4cc5ccccc5c5ccccc45)c4ccccc24)cc13 ; c1ccc2cc3c(cc2c1)oc1ccc(-c2c4ccccc4c(-c4ccc(-c5cccc6ccccc56)cc4)c4ccccc24)cc13 ; c1ccc2cc(-c3ccc(-c4c5ccccc5c(-c5ccc6oc7cc8ccccc8cc7c6c5)c5ccccc45)cc3)ccc2c1]

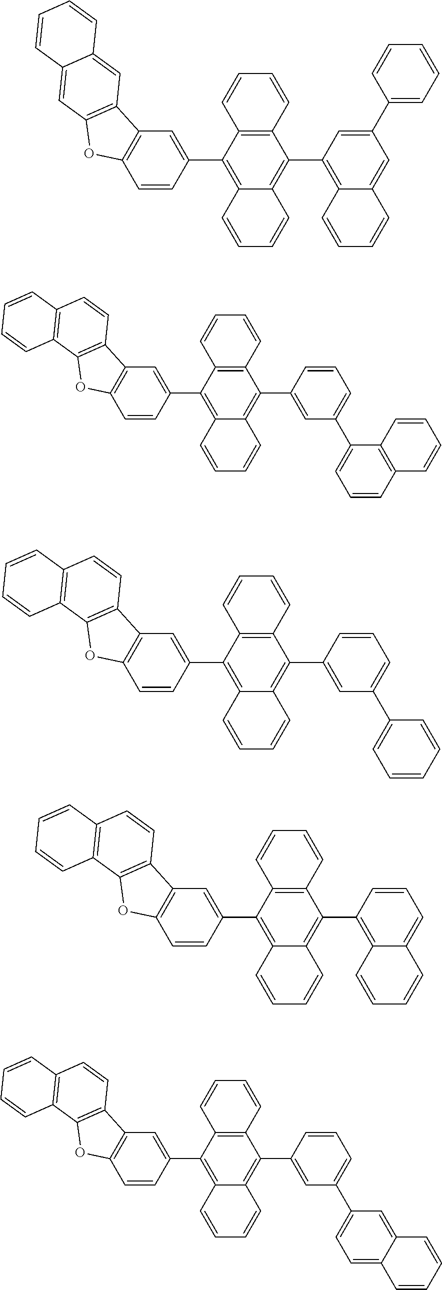 [SMILES: c1ccc(-c2cc(-c3c4ccccc4c(-c4ccc5oc6cc7ccccc7cc6c5c4)c4ccccc34)c3ccccc3c2)cc1 ; c1cc(-c2cccc3ccccc23)cc(-c2c3ccccc3c(-c3ccc4oc5c6ccccc6ccc5c4c3)c3ccccc23)c1 ; c1ccc(-c2cccc(-c3c4ccccc4c(-c4ccc5oc6c7ccccc7ccc6c5c4)c4ccccc34)c2)cc1 ; c1ccc2c(-c3c4ccccc4c(-c4ccc5oc6c7ccccc7ccc6c5c4)c4ccccc34)cccc2c1 ; c1cc(-c2ccc3ccccc3c2)cc(-c2c3ccccc3c(-c3ccc4oc5c6ccccc6ccc5c4c3)c3ccccc23)c1]

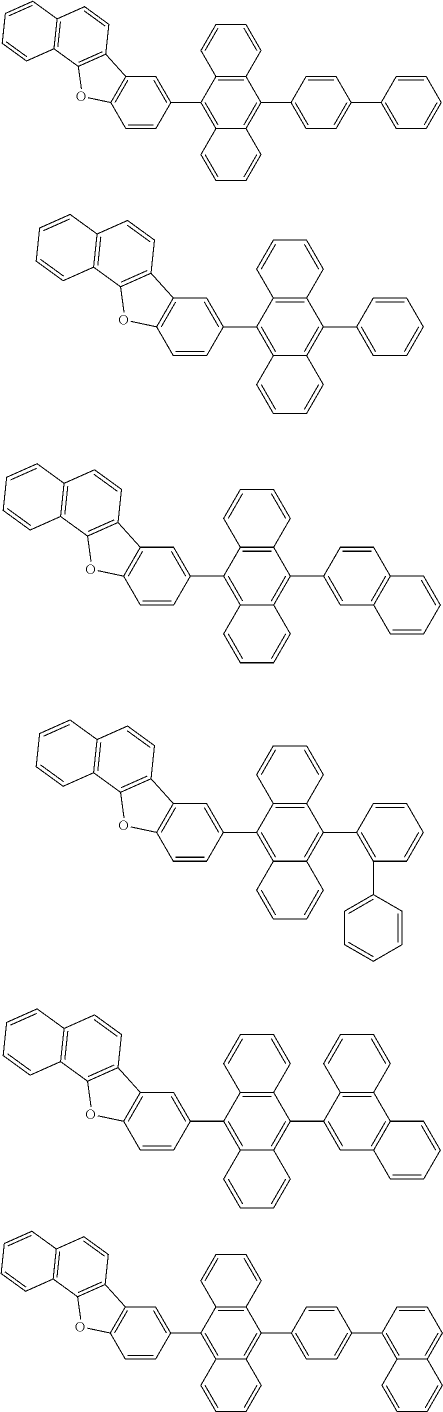 [SMILES: c1ccc(-c2ccc(-c3c4ccccc4c(-c4ccc5oc6c7ccccc7ccc6c5c4)c4ccccc34)cc2)cc1 ; c1ccc(-c2c3ccccc3c(-c3ccc4oc5c6ccccc6ccc5c4c3)c3ccccc23)cc1 ; c1ccc2cc(-c3c4ccccc4c(-c4ccc5oc6c7ccccc7ccc6c5c4)c4ccccc34)ccc2c1 ; c1ccc(-c2ccccc2-c2c3ccccc3c(-c3ccc4oc5c6ccccc6ccc5c4c3)c3ccccc23)cc1 ; c1ccc2c(c1)cc(-c1c3ccccc3c(-c3ccc4oc5c6ccccc6ccc5c4c3)c3ccccc13)c1ccccc12 ; c1ccc2c(-c3ccc(-c4c5ccccc5c(-c5ccc6oc7c8ccccc8ccc7c6c5)c5ccccc45)cc3)cccc2c1]

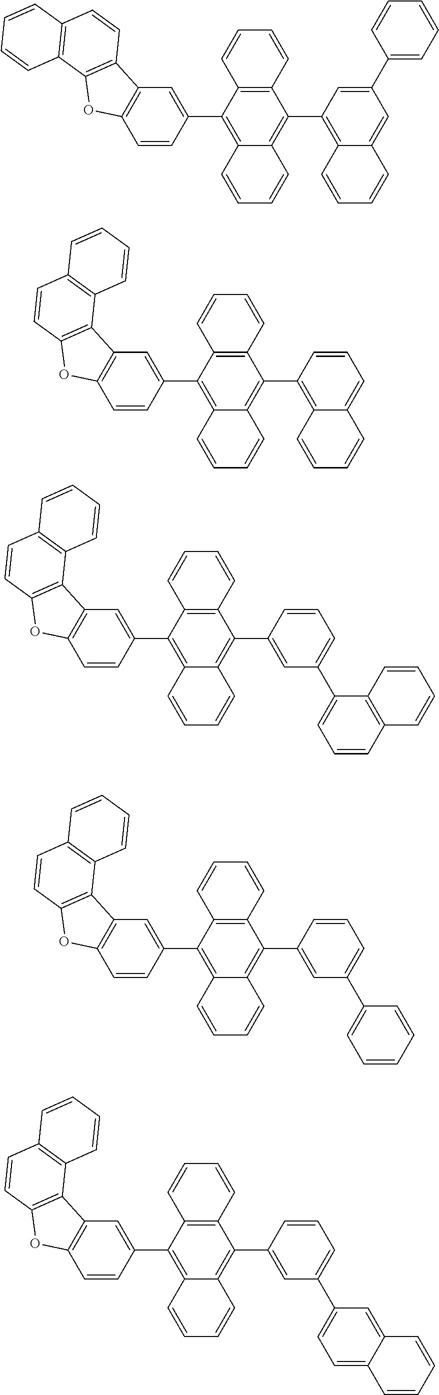 [SMILES: c1ccc(-c2cc(-c3c4ccccc4c(-c4ccc5oc6c7ccccc7ccc6c5c4)c4ccccc34)c3ccccc3c2)cc1 ; c1ccc2c(-c3c4ccccc4c(-c4ccc5oc6ccc7ccccc7c6c5c4)c4ccccc34)cccc2c1 ; c1cc(-c2cccc3ccccc23)cc(-c2c3ccccc3c(-c3ccc4oc5ccc6ccccc6c5c4c3)c3ccccc23)c1 ; c1ccc(-c2cccc(-c3c4ccccc4c(-c4ccc5oc6ccc7ccccc7c6c5c4)c4ccccc34)c2)cc1 ; c1cc(-c2ccc3ccccc3c2)cc(-c2c3ccccc3c(-c3ccc4oc5ccc6ccccc6c5c4c3)c3ccccc23)c1]

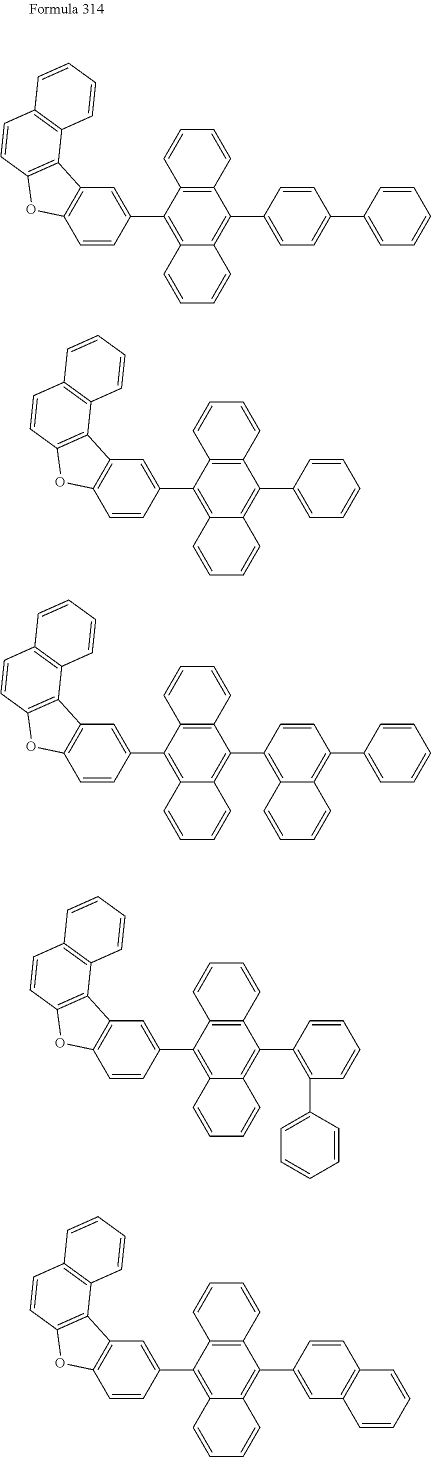 [SMILES: c1ccc(-c2ccc(-c3c4ccccc4c(-c4ccc5oc6ccc7ccccc7c6c5c4)c4ccccc34)cc2)cc1 ; c1ccc(-c2c3ccccc3c(-c3ccc4oc5ccc6ccccc6c5c4c3)c3ccccc23)cc1 ; c1ccc(-c2ccc(-c3c4ccccc4c(-c4ccc5oc6ccc7ccccc7c6c5c4)c4ccccc34)c3ccccc23)cc1 ; c1ccc(-c2ccccc2-c2c3ccccc3c(-c3ccc4oc5ccc6ccccc6c5c4c3)c3ccccc23)cc1 ; c1ccc2cc(-c3c4ccccc4c(-c4ccc5oc6ccc7ccccc7c6c5c4)c4ccccc34)ccc2c1]

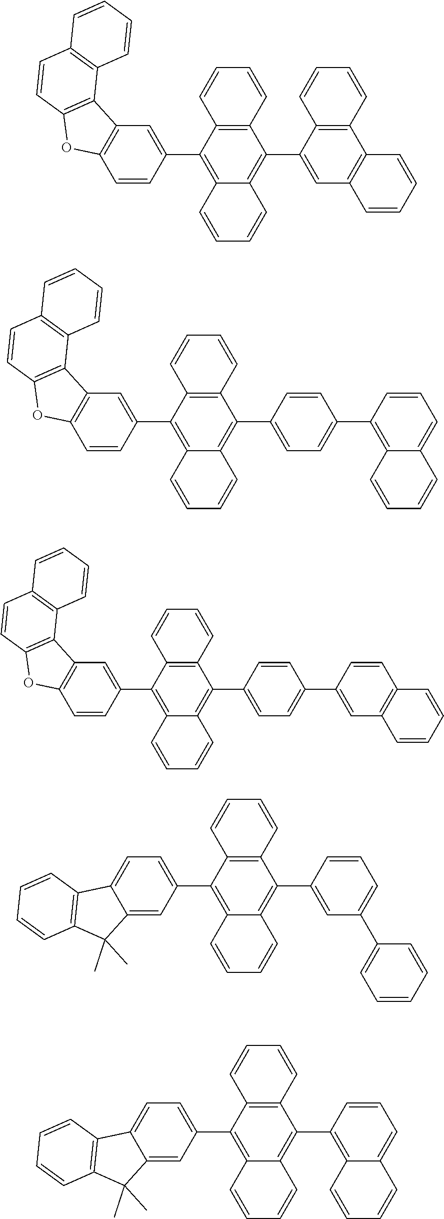

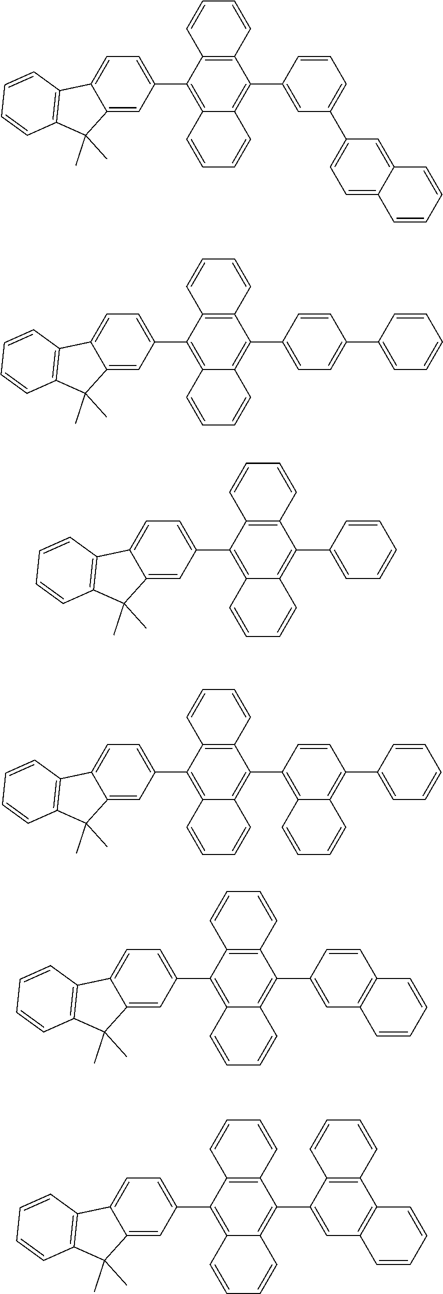

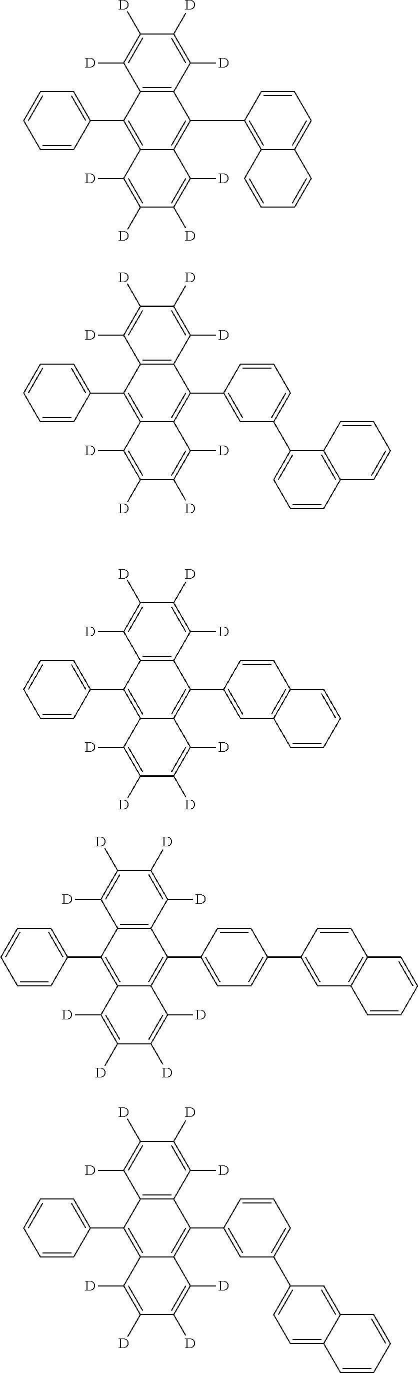

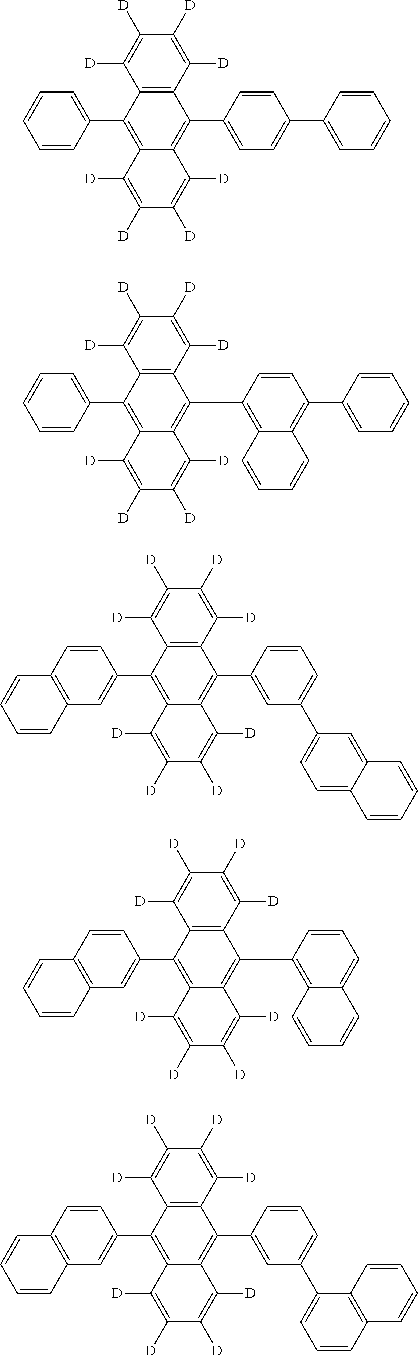

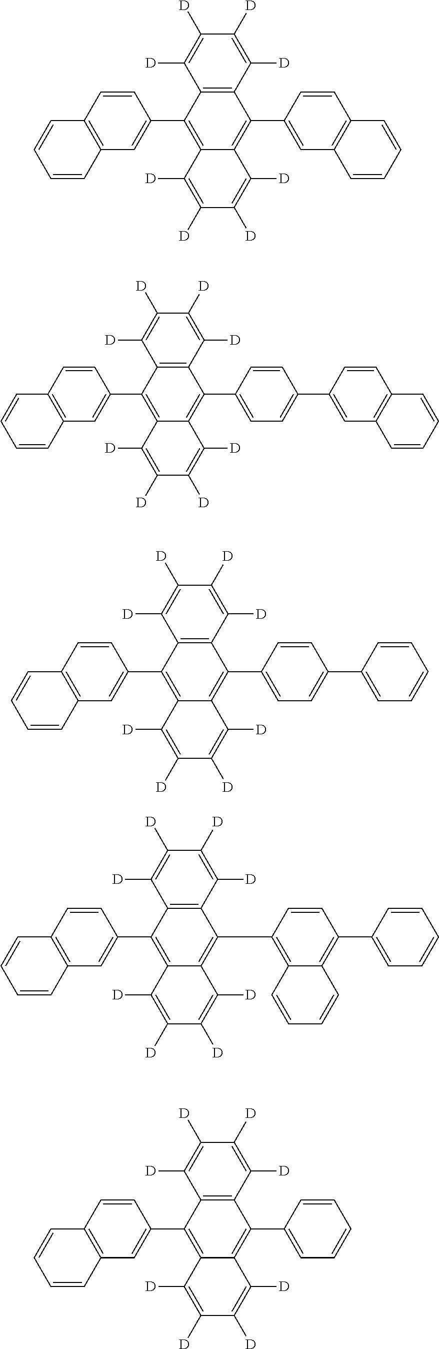

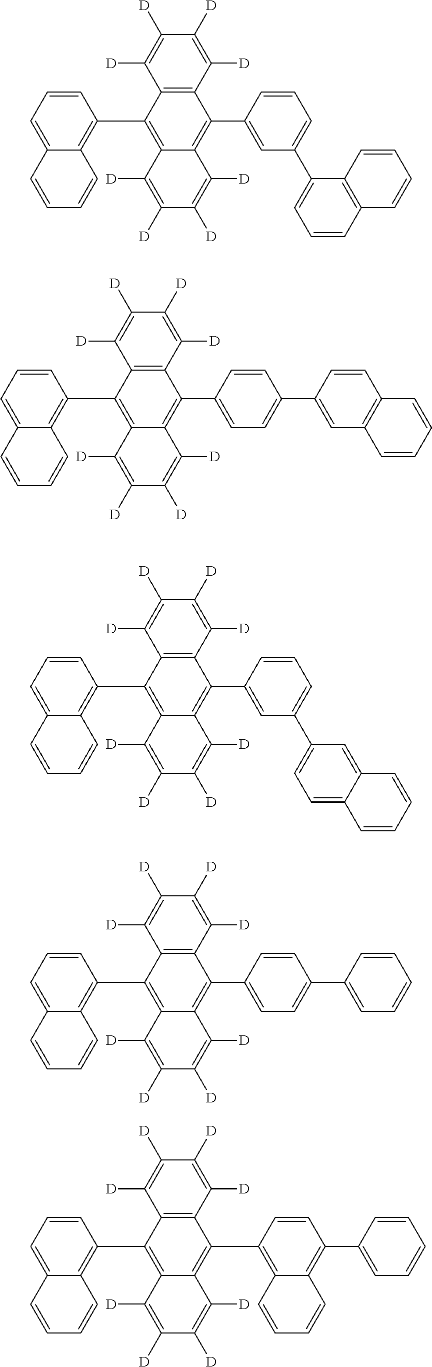

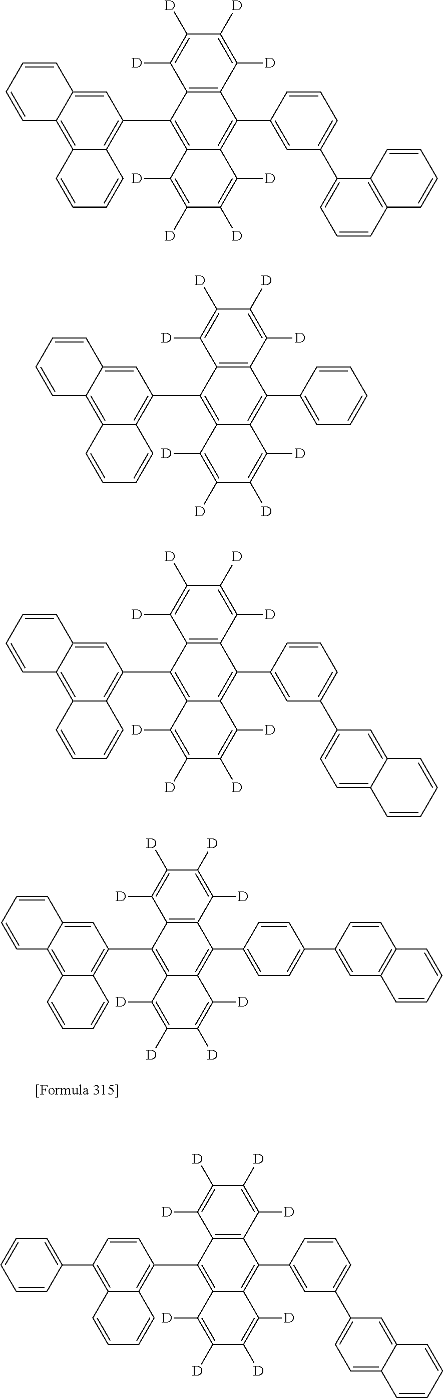

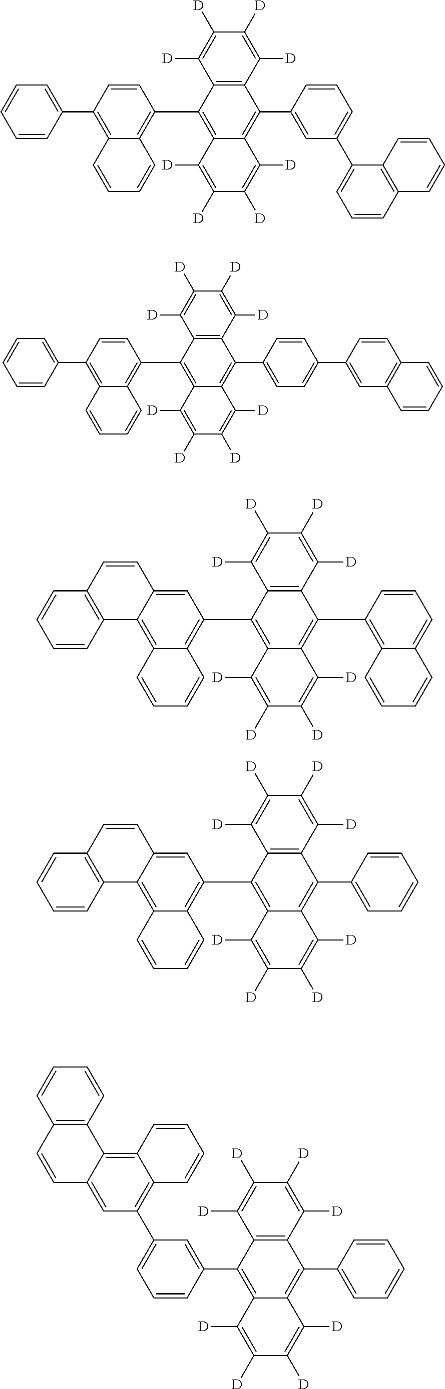

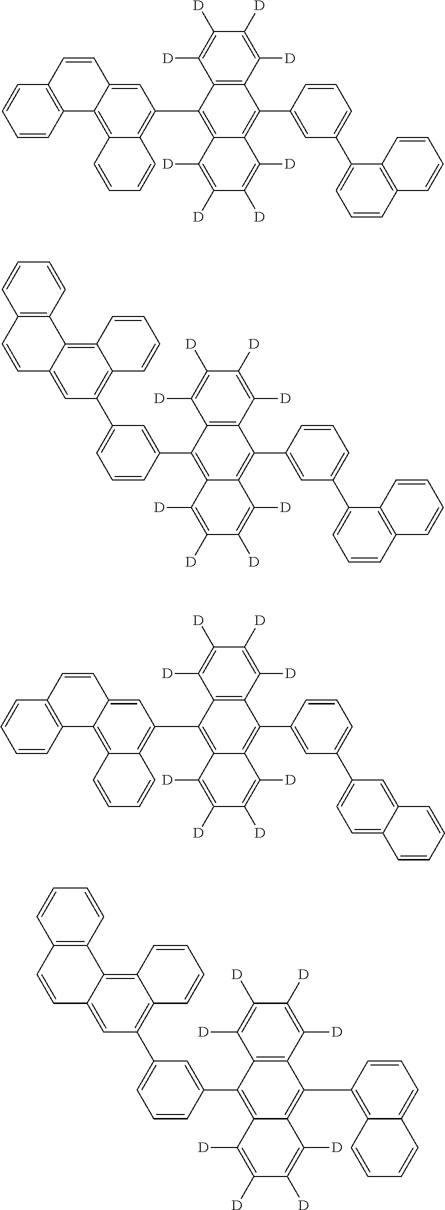

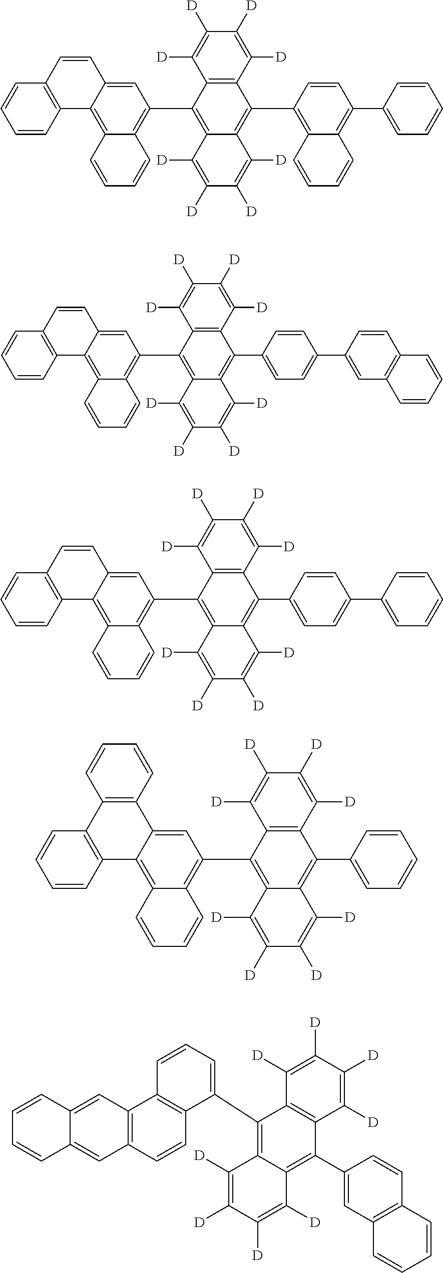

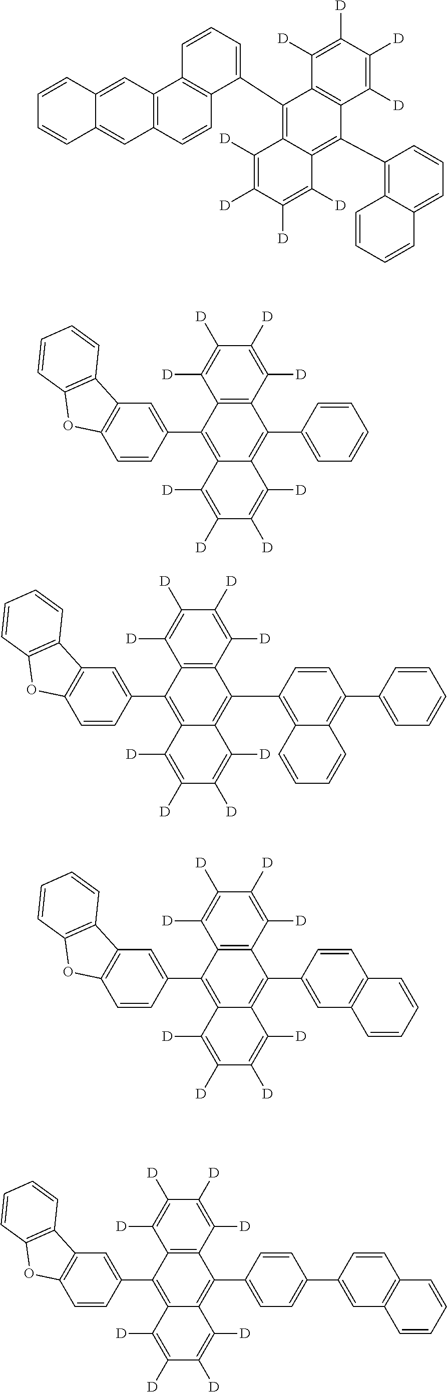

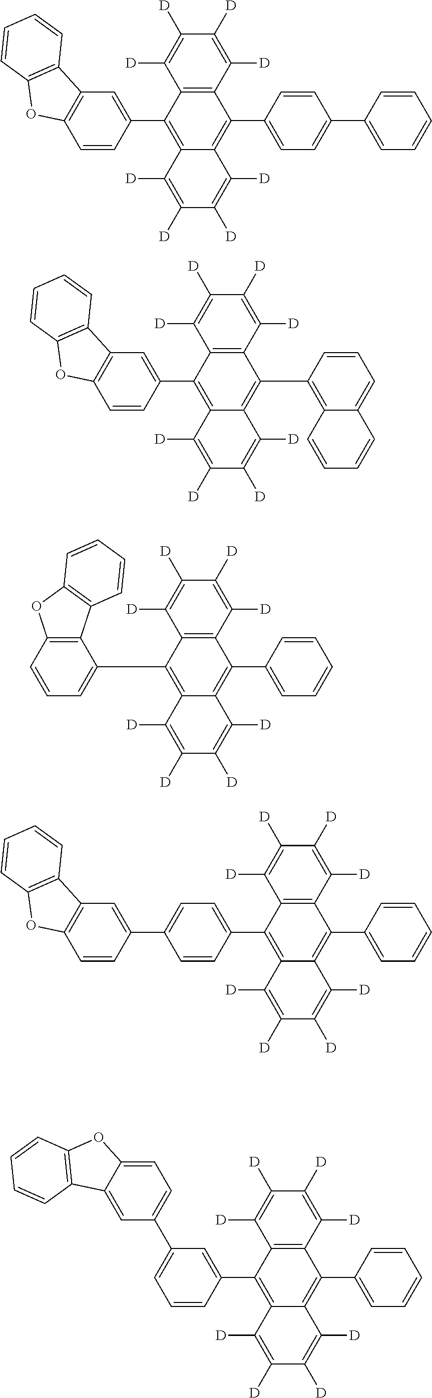

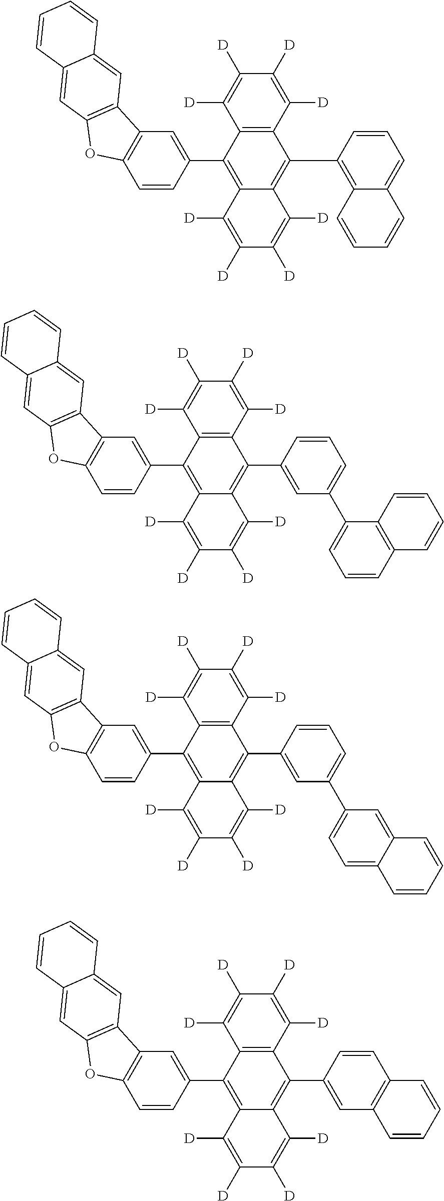 [SMILES: [2H]c1c([2H])c([2H])c2c(-c3cccc4ccccc34)c3c([2H])c([2H])c([2H])c([2H])c3c(-c3ccc4oc5cc6ccccc6cc5c4c3)c2c1[2H] ; [2H]c1c([2H])c([2H])c2c(-c3ccc4oc5cc6ccccc6cc5c4c3)c3c([2H])c([2H])c([2H])c([2H])c3c(-c3cccc(-c4cccc5ccccc45)c3)c2c1[2H] ; [2H]c1c([2H])c([2H])c2c(-c3ccc4oc5cc6ccccc6cc5c4c3)c3c([2H])c([2H])c([2H])c([2H])c3c(-c3cccc(-c4ccc5ccccc5c4)c3)c2c1[2H] ; [2H]c1c([2H])c([2H])c2c(-c3ccc4oc5cc6ccccc6cc5c4c3)c3c([2H])c([2H])c([2H])c([2H])c3c(-c3ccc4ccccc4c3)c2c1[2H]]

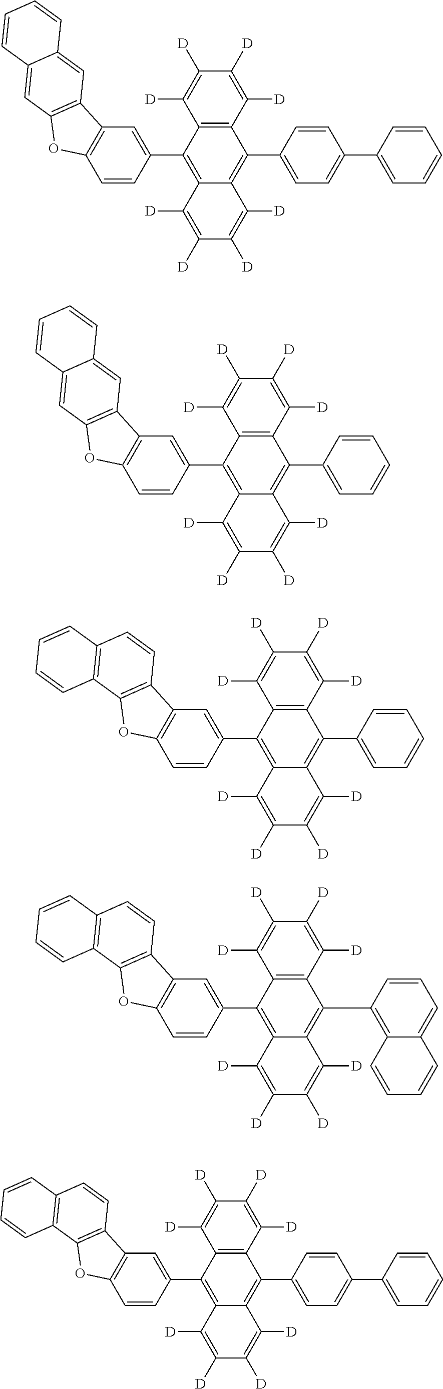

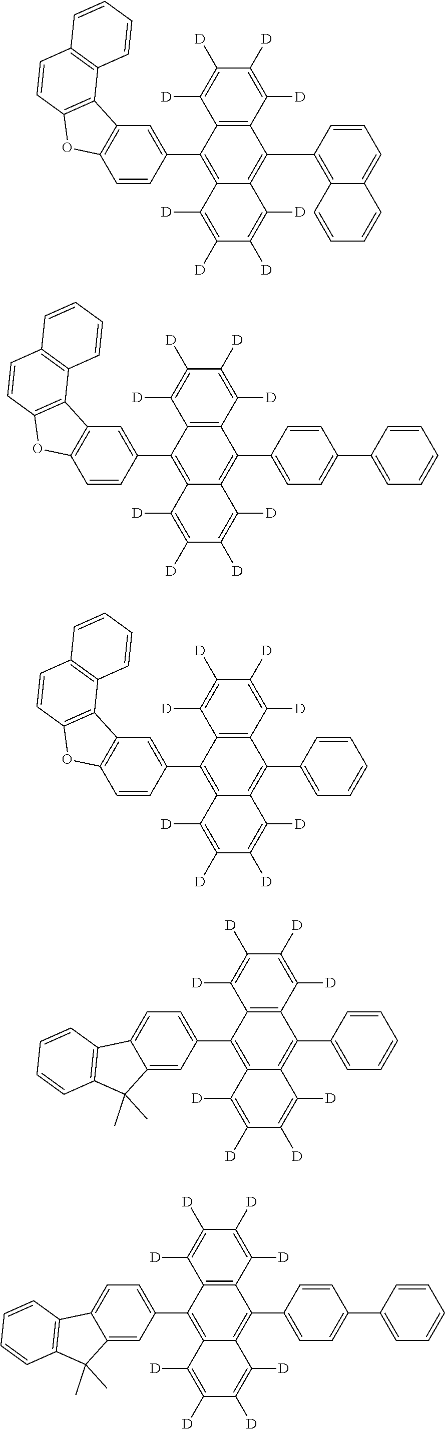

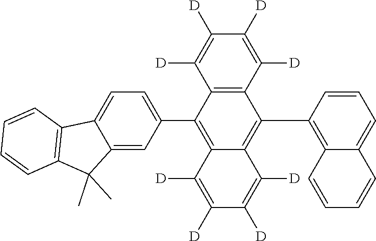 [SMILES: [2H]c1c([2H])c([2H])c2c(-c3cccc4ccccc34)c3c([2H])c([2H])c([2H])c([2H])c3c(-c3ccc4c(c3)C(C)(C)c3ccccc3-4)c2c1[2H]]

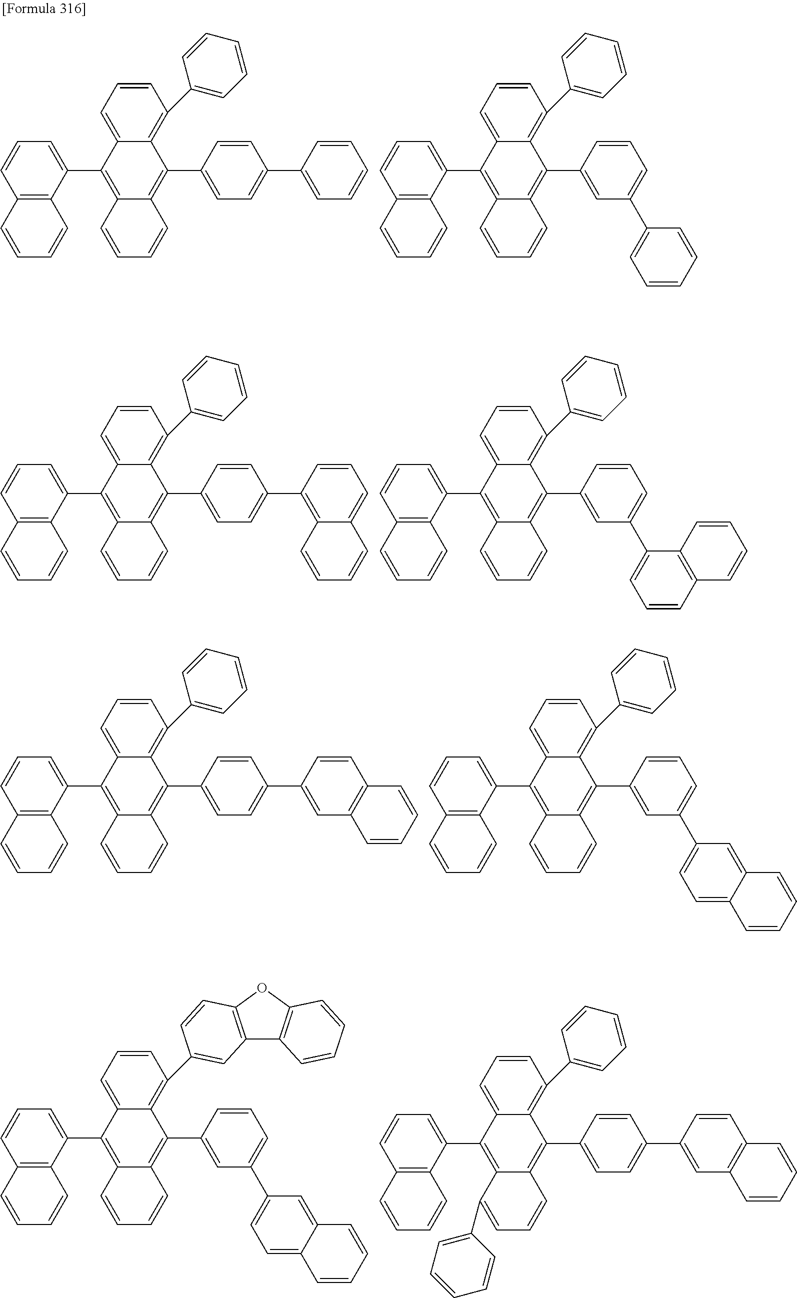

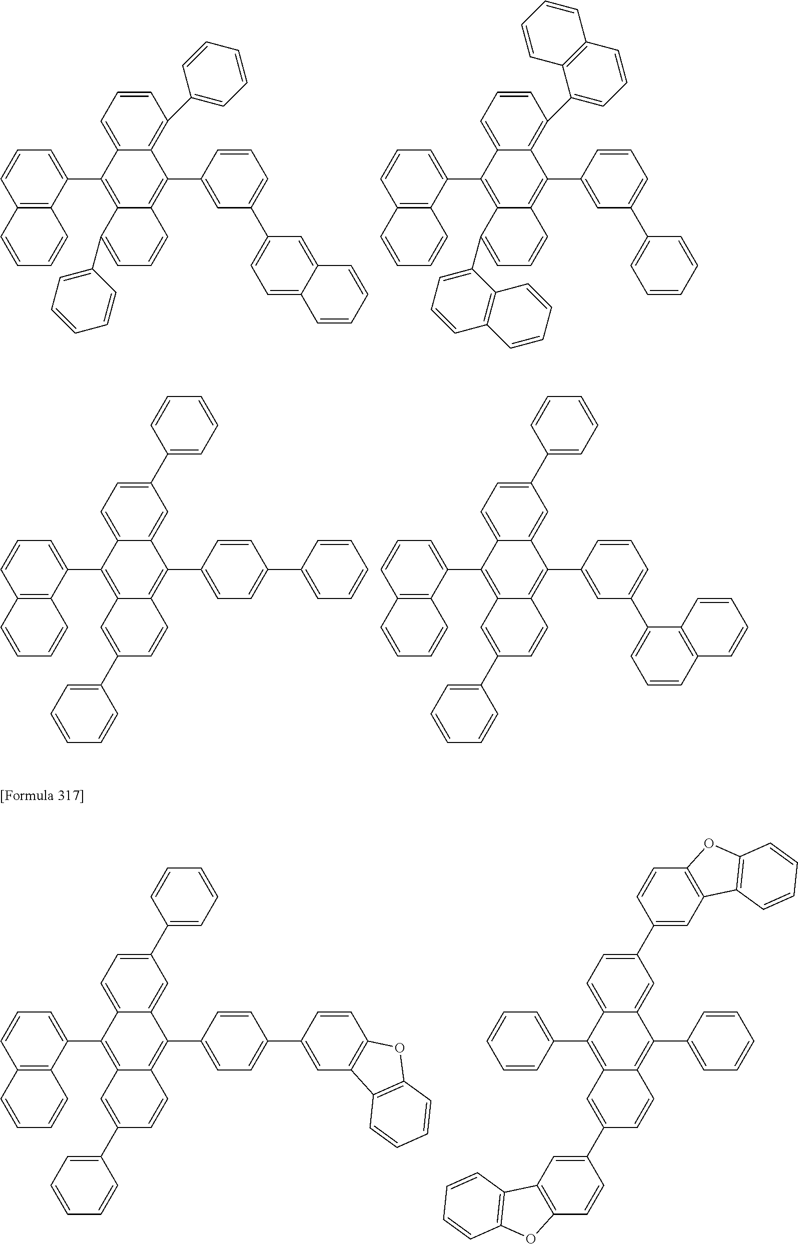

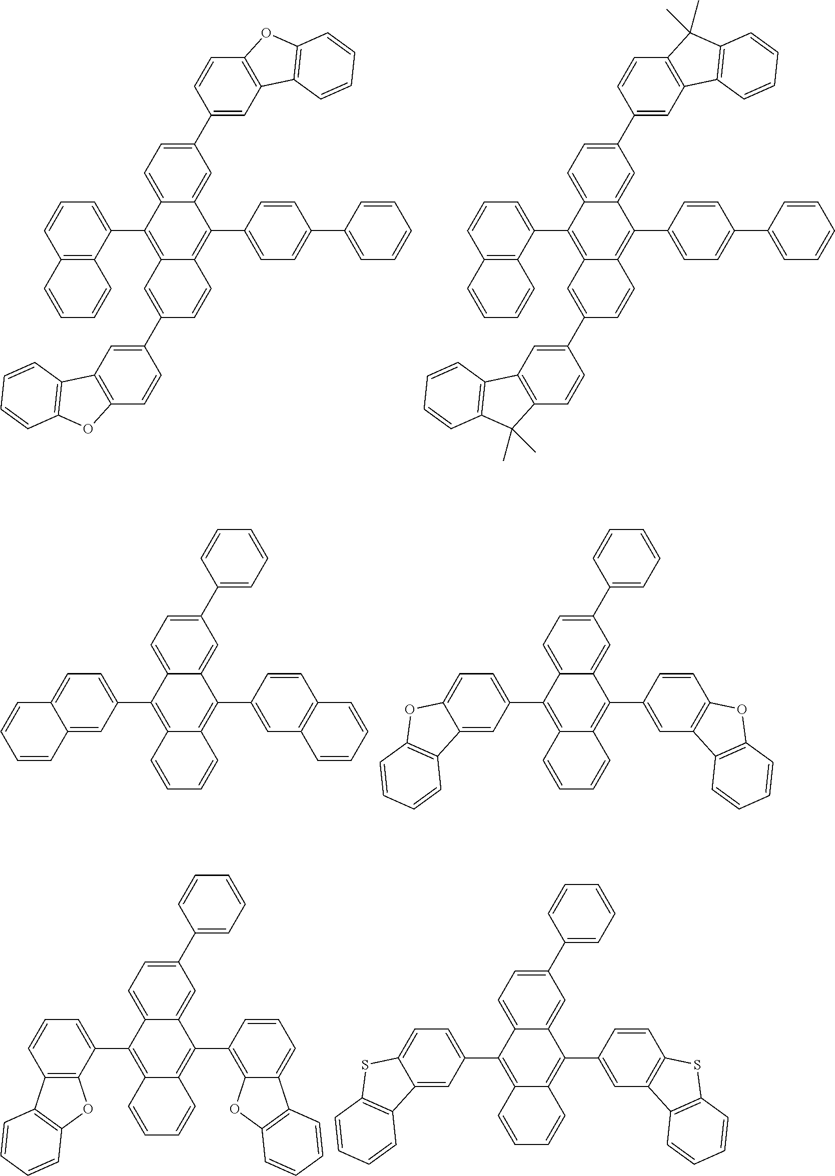

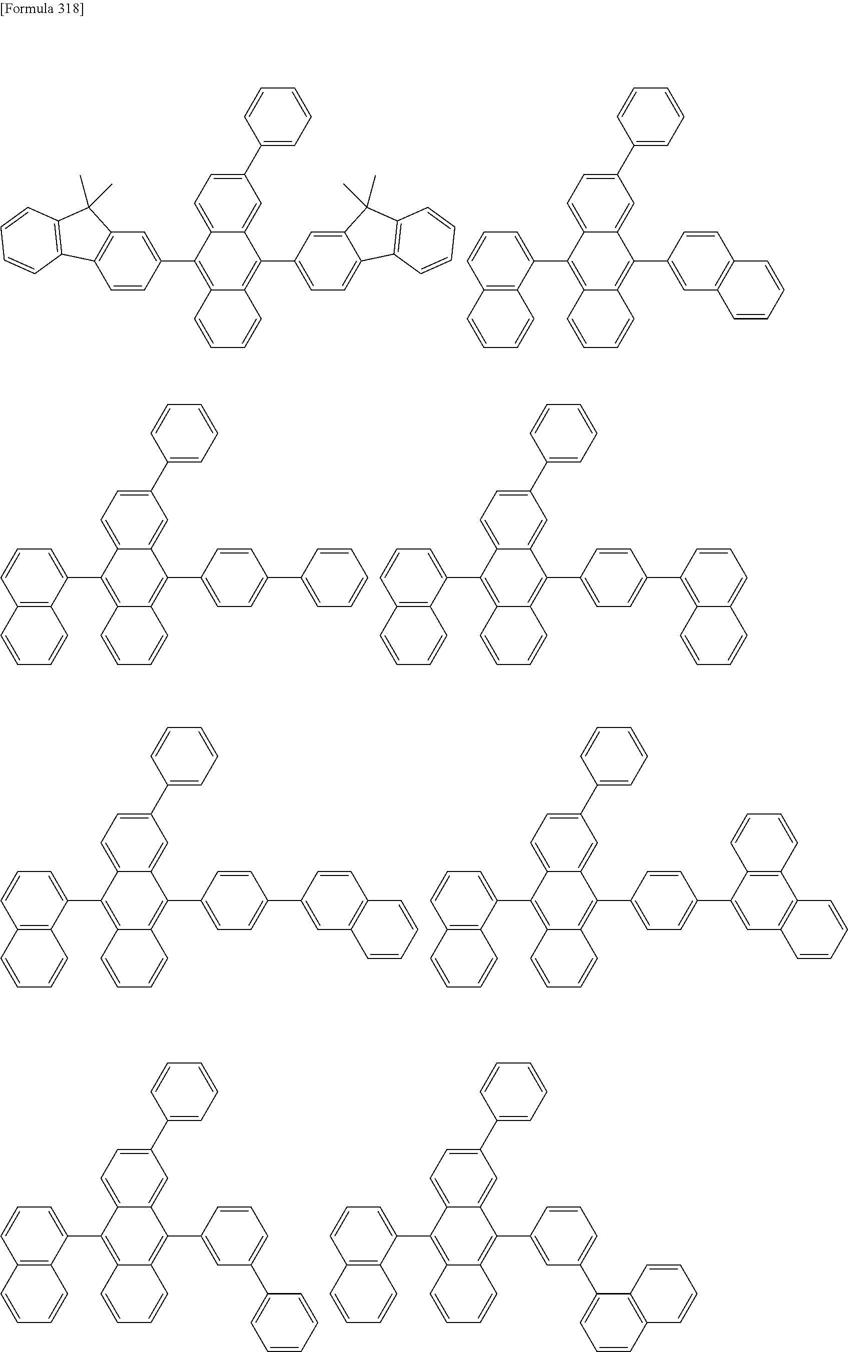

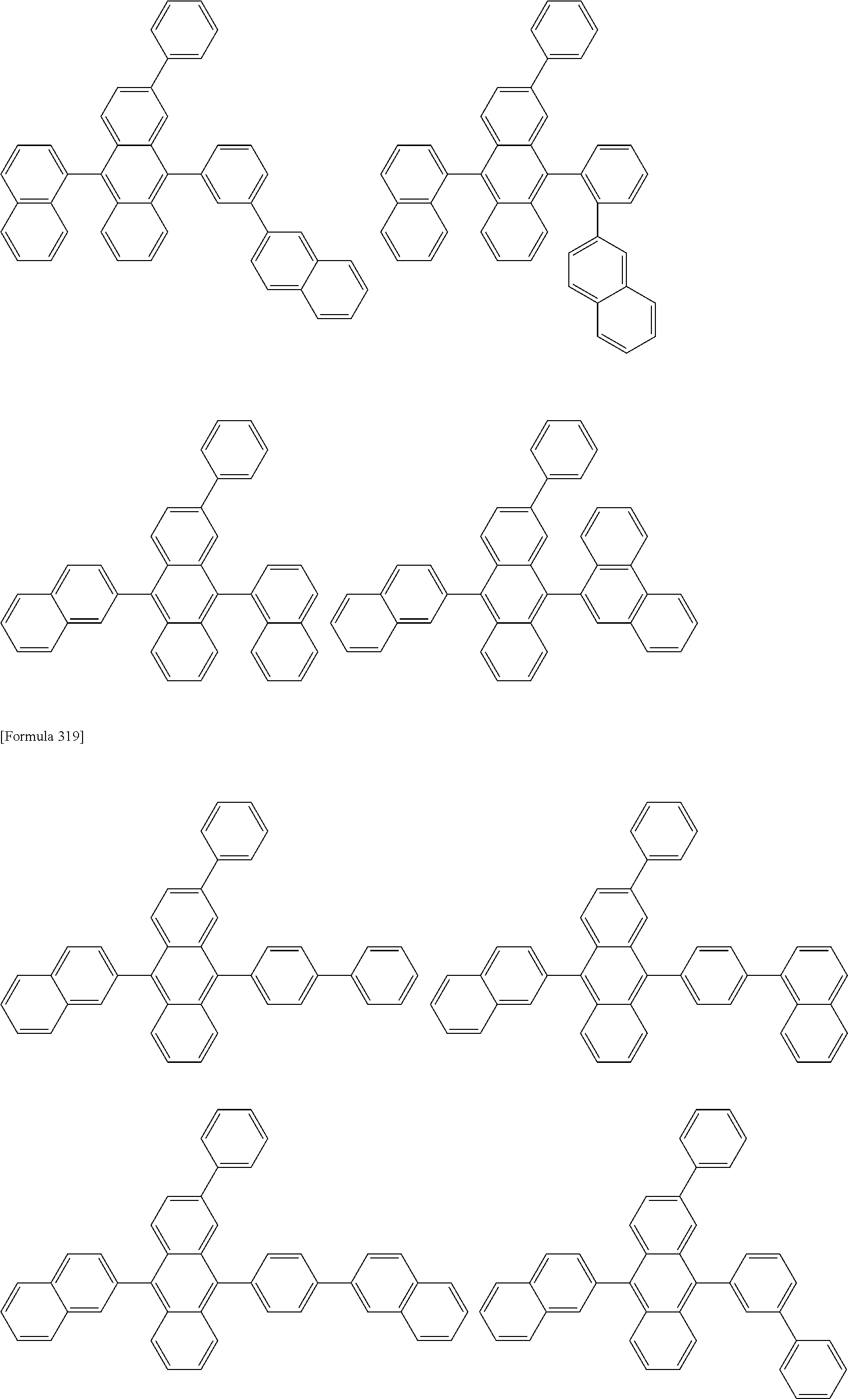

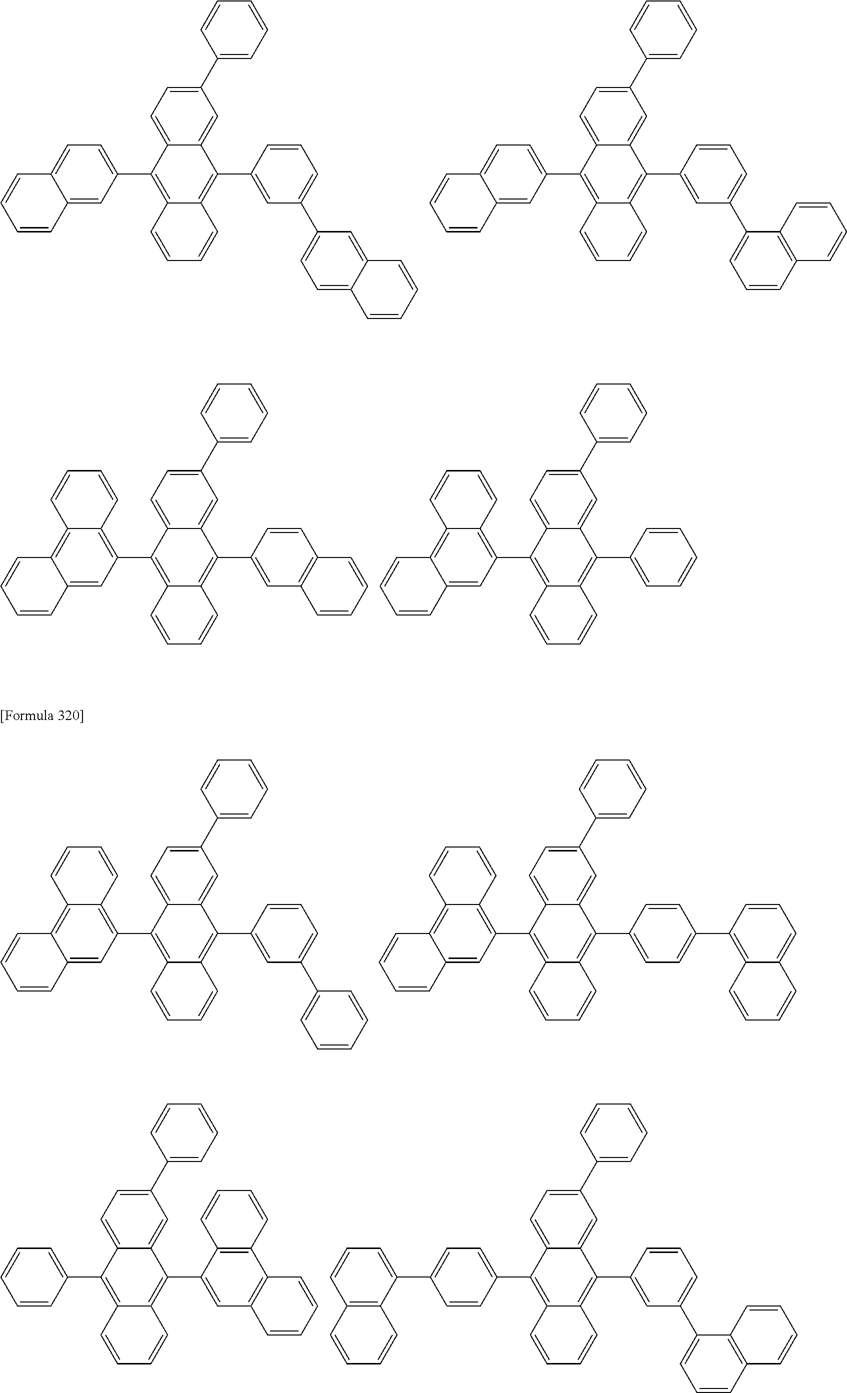

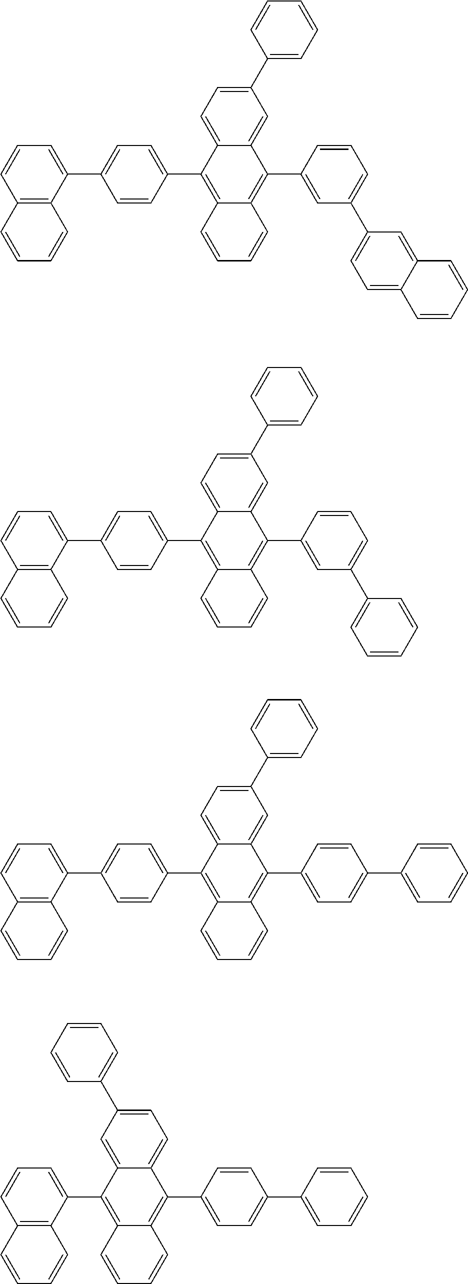

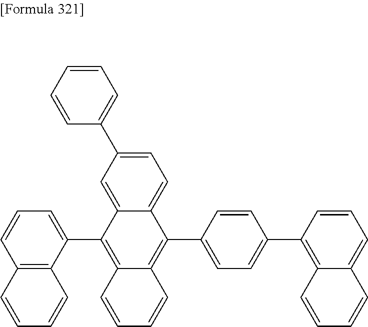 [SMILES: c1ccc(-c2ccc3c(-c4ccc(-c5cccc6ccccc56)cc4)c4ccccc4c(-c4cccc5ccccc45)c3c2)cc1]

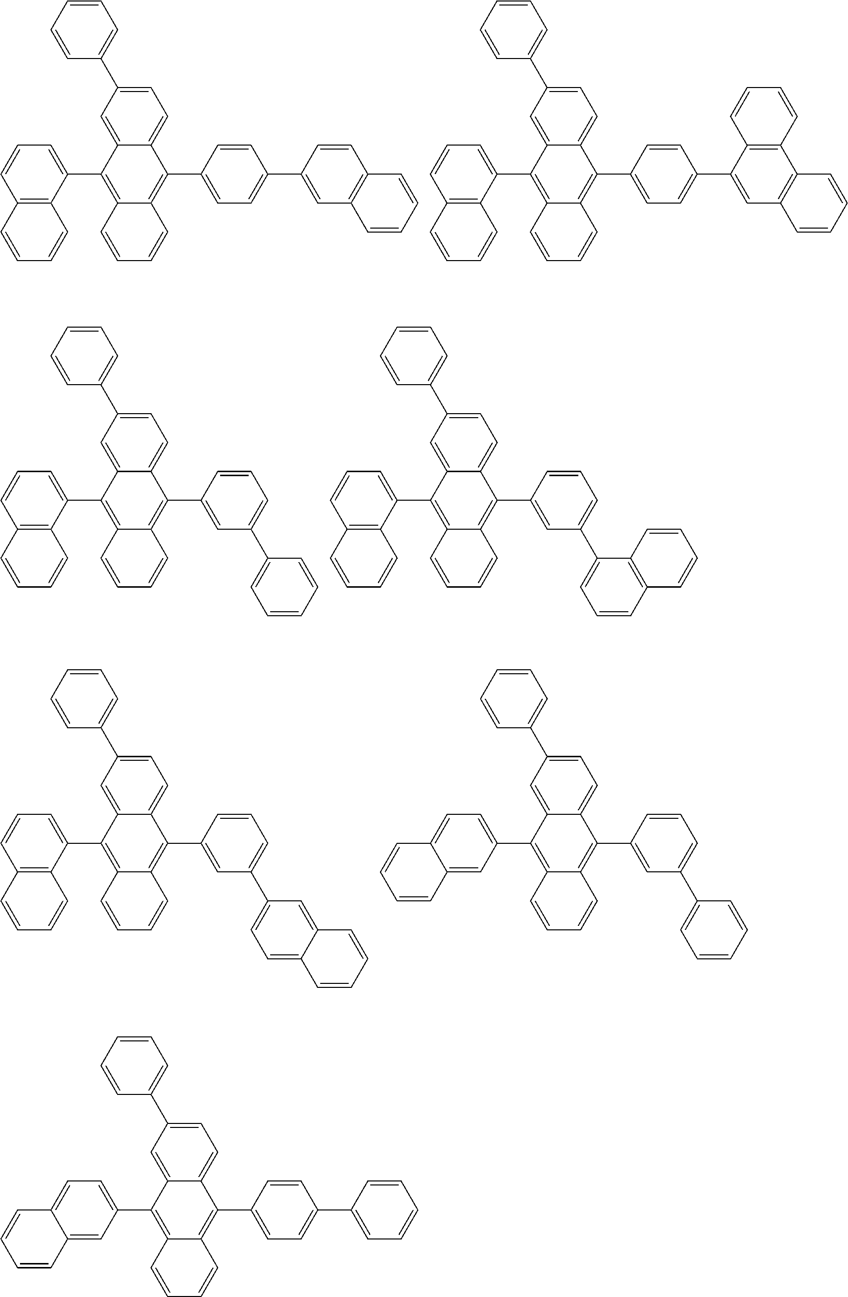

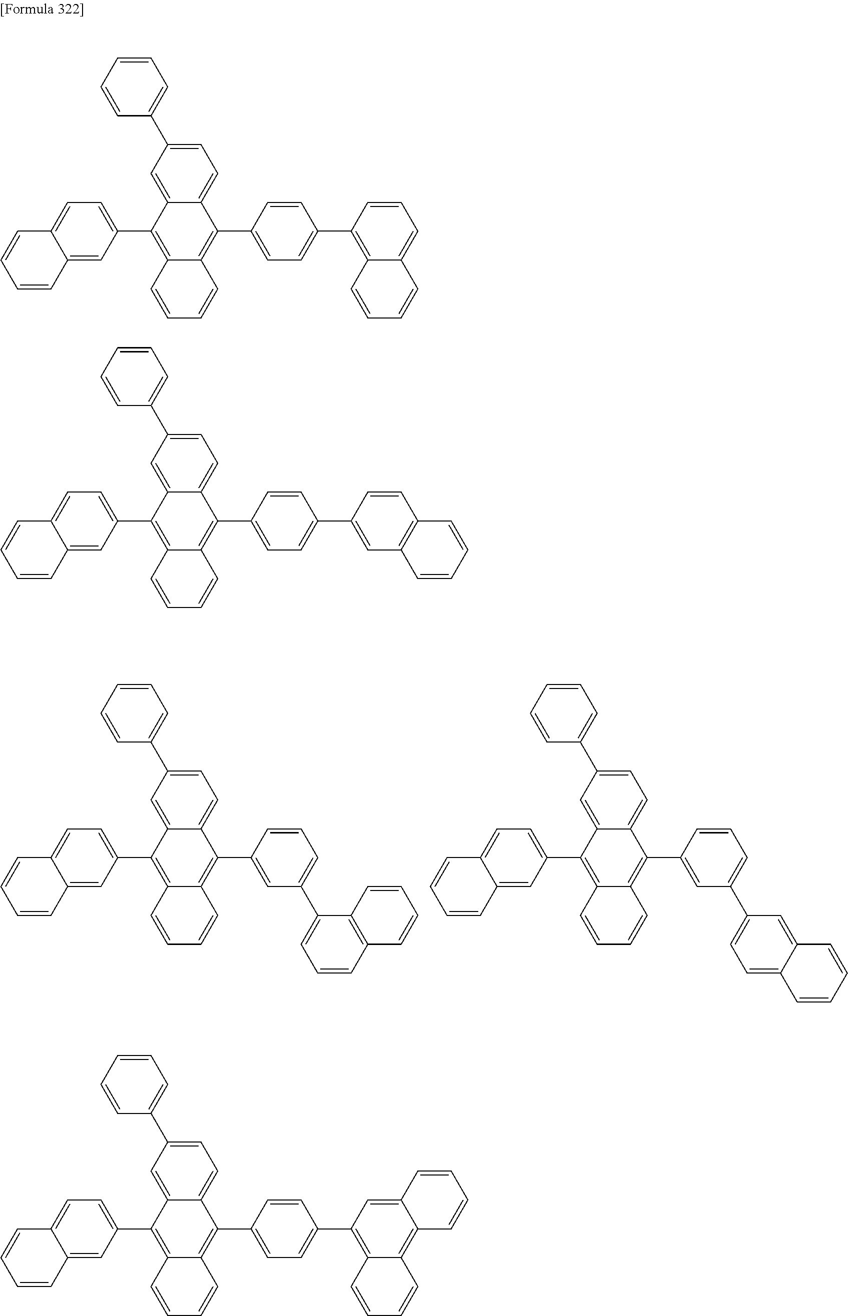

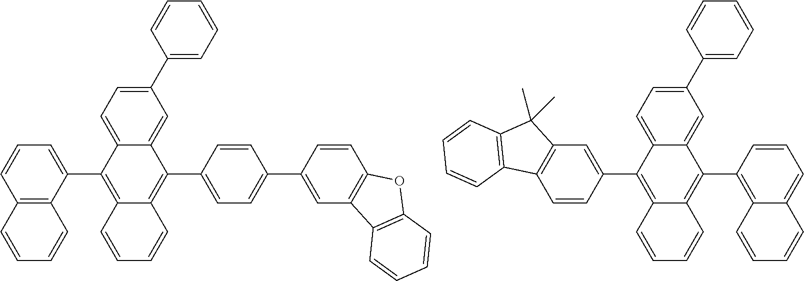

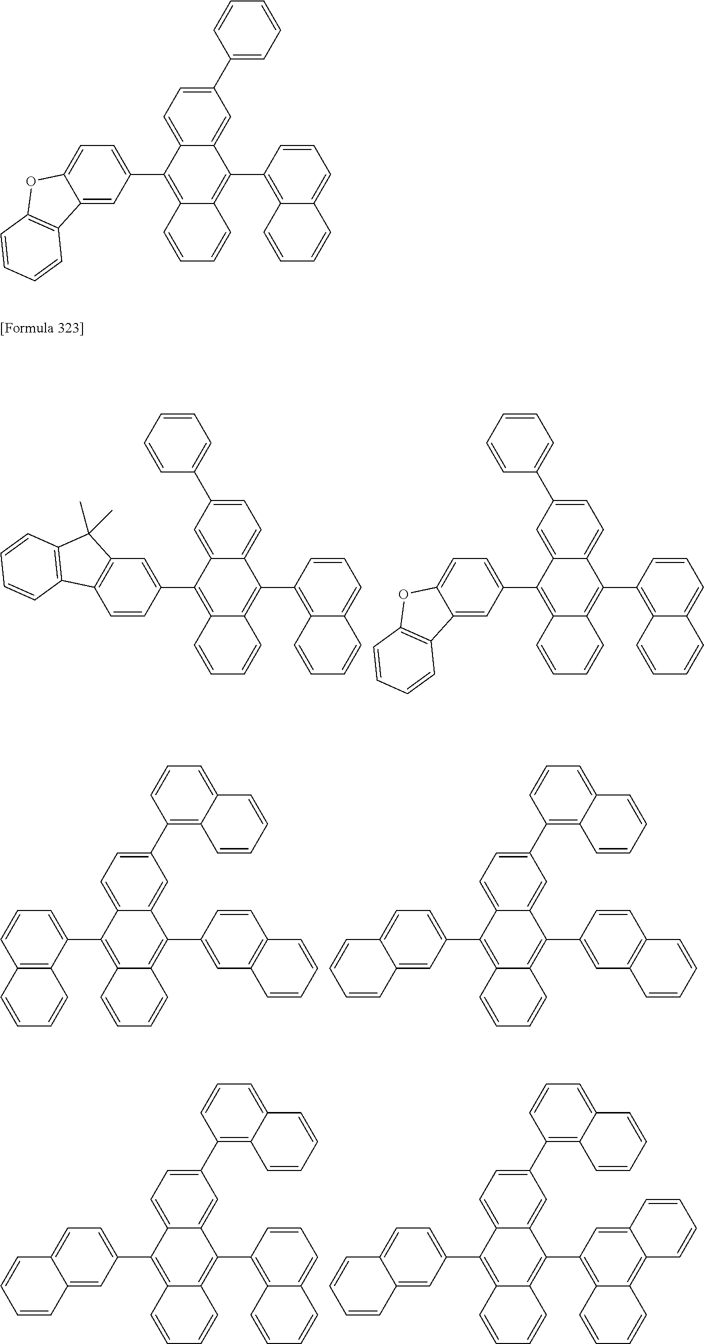

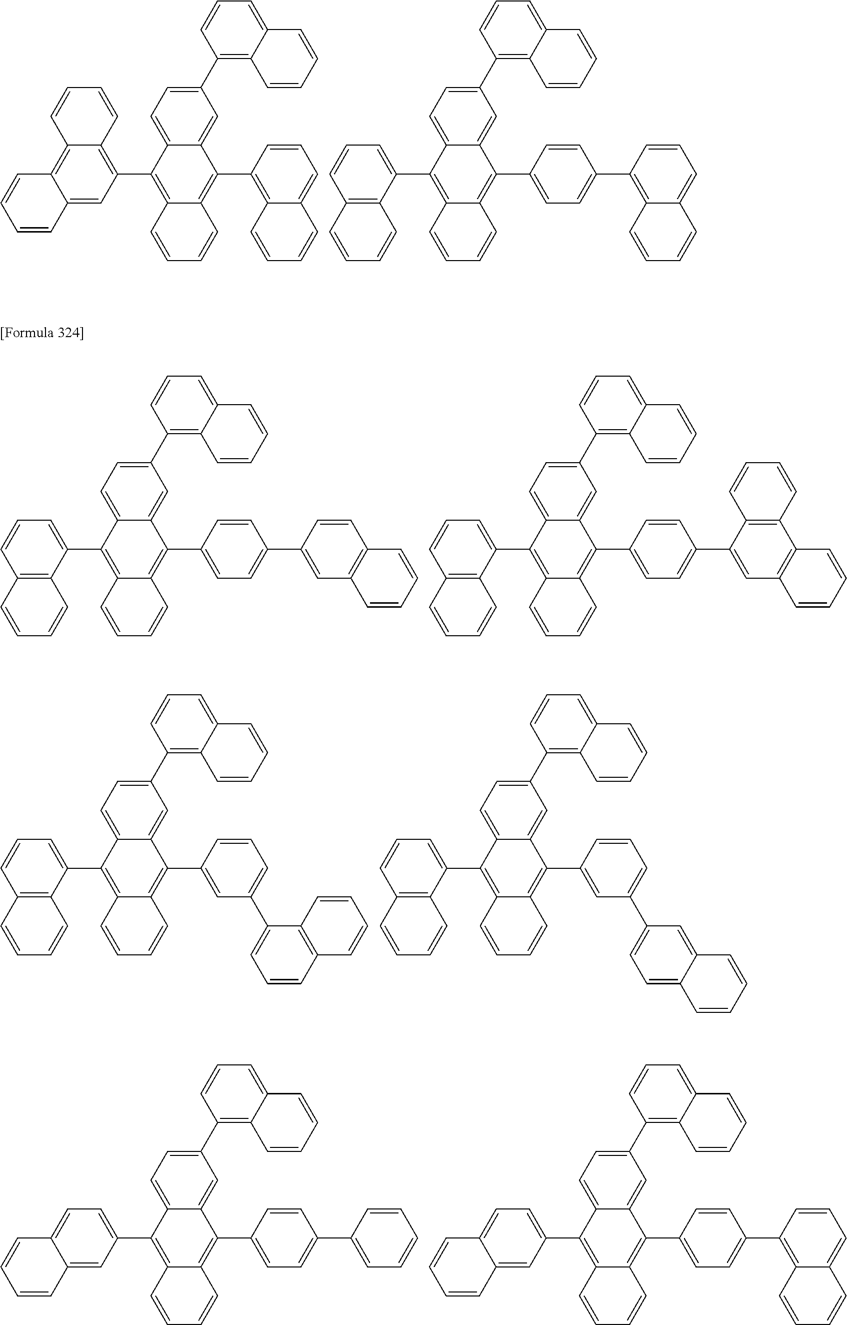

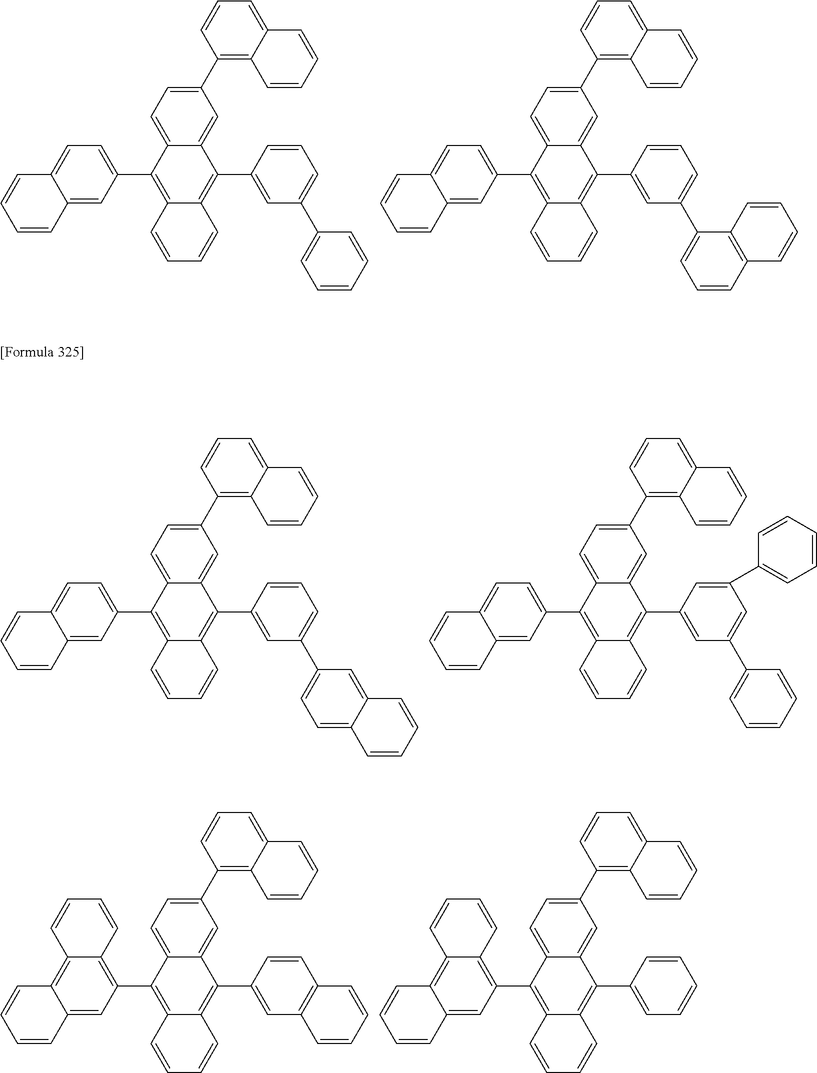

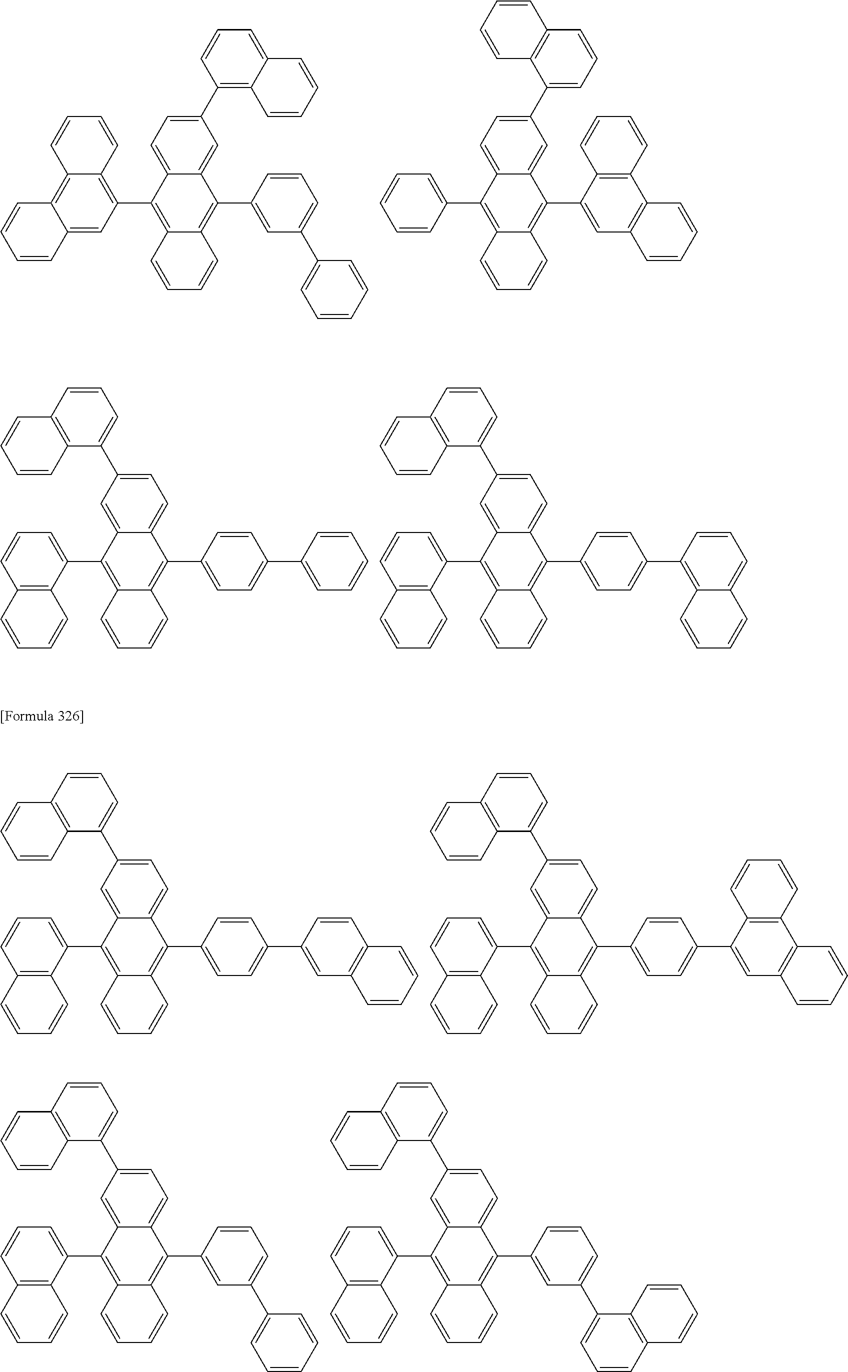

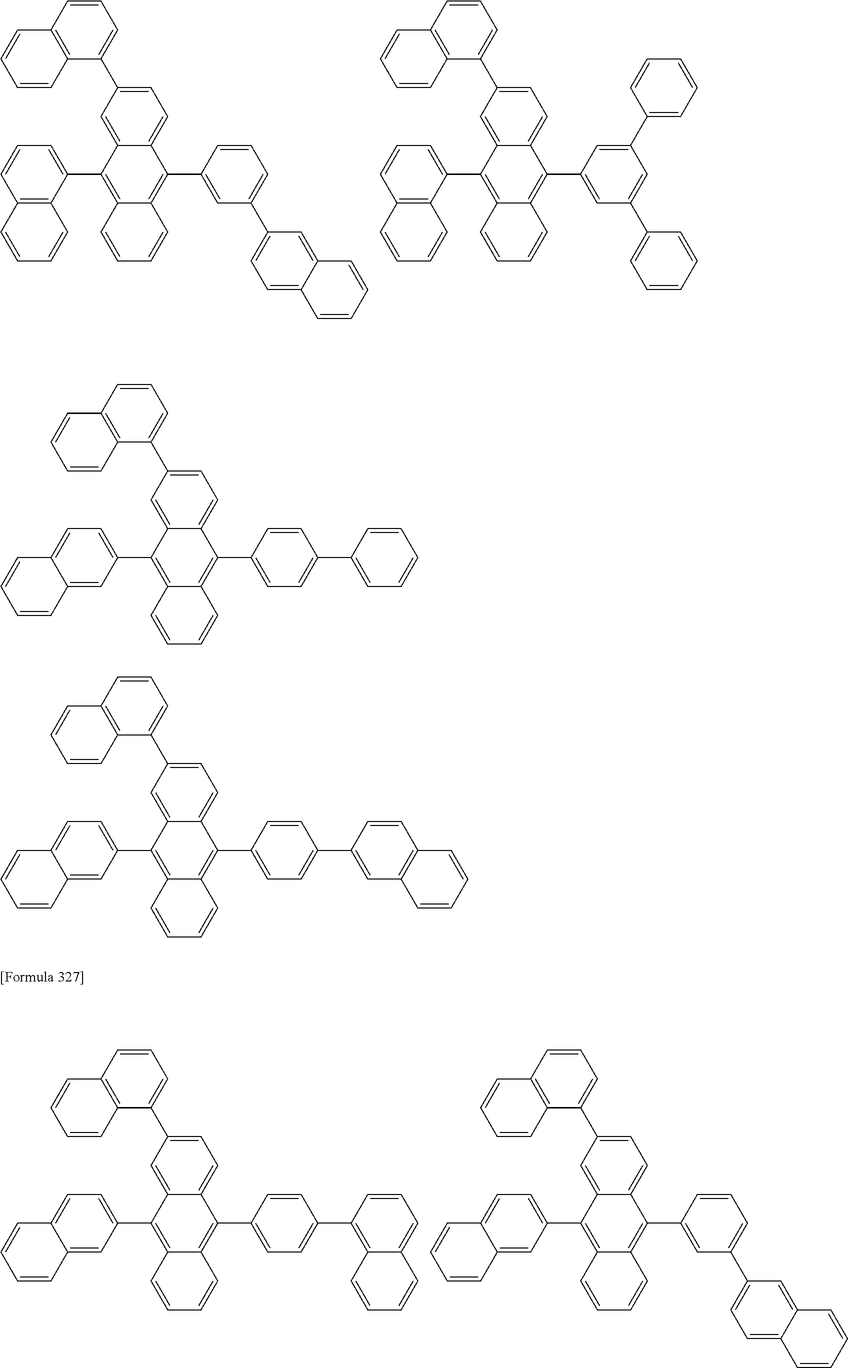

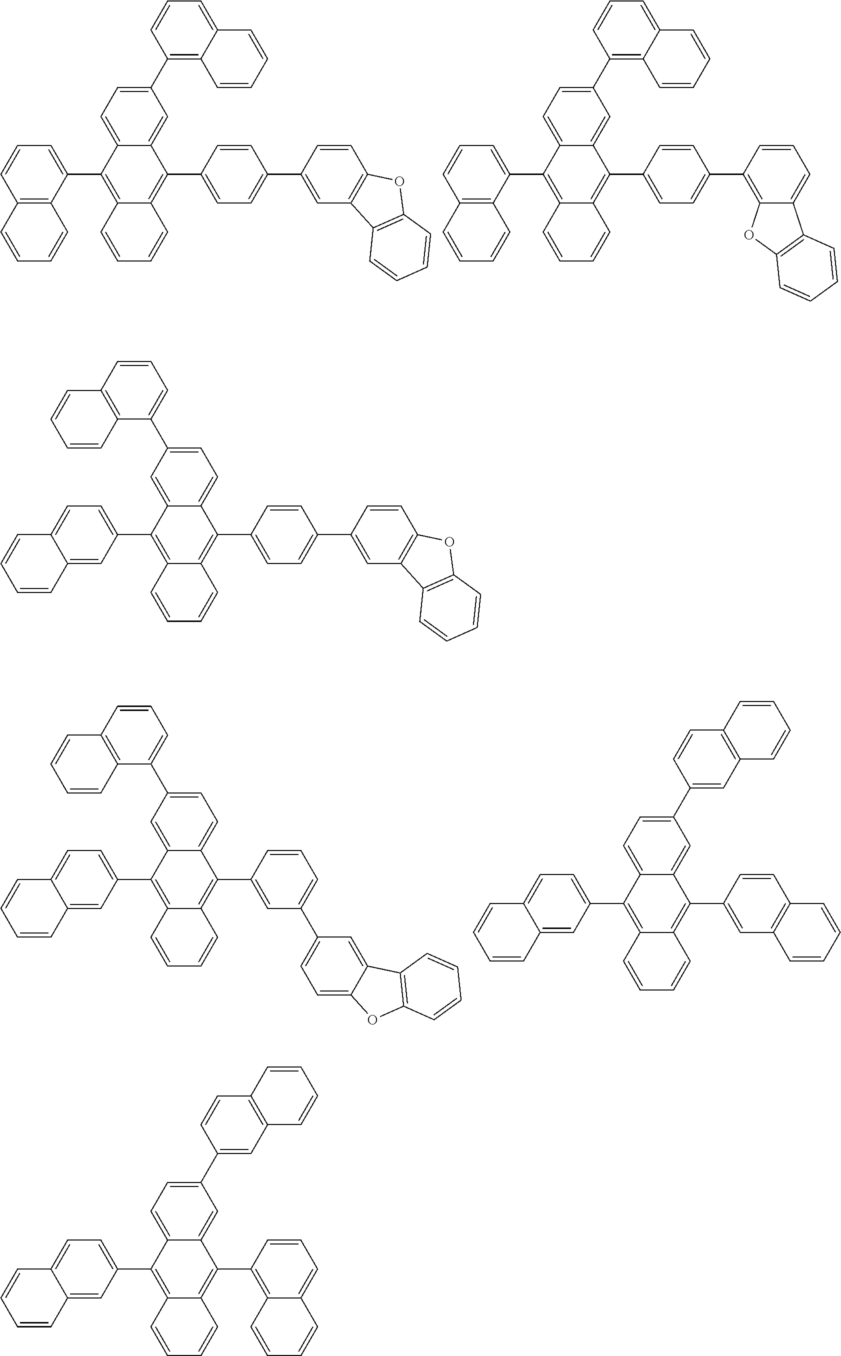

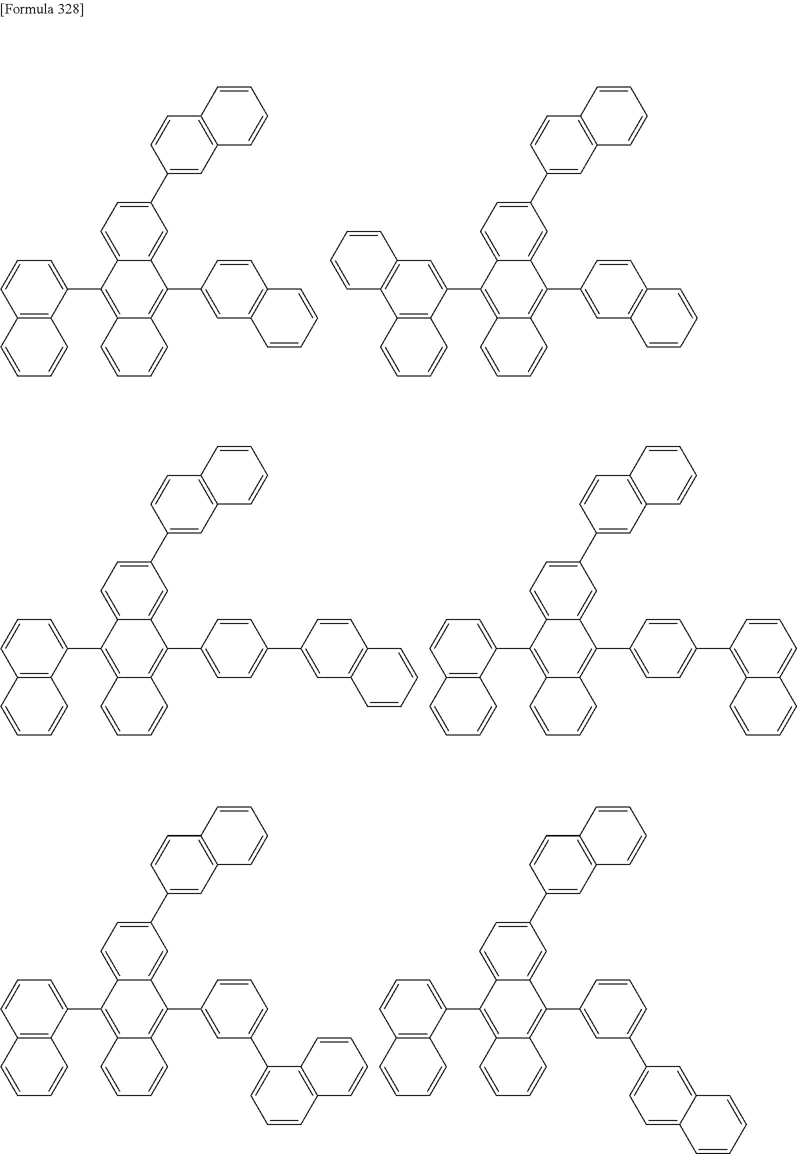

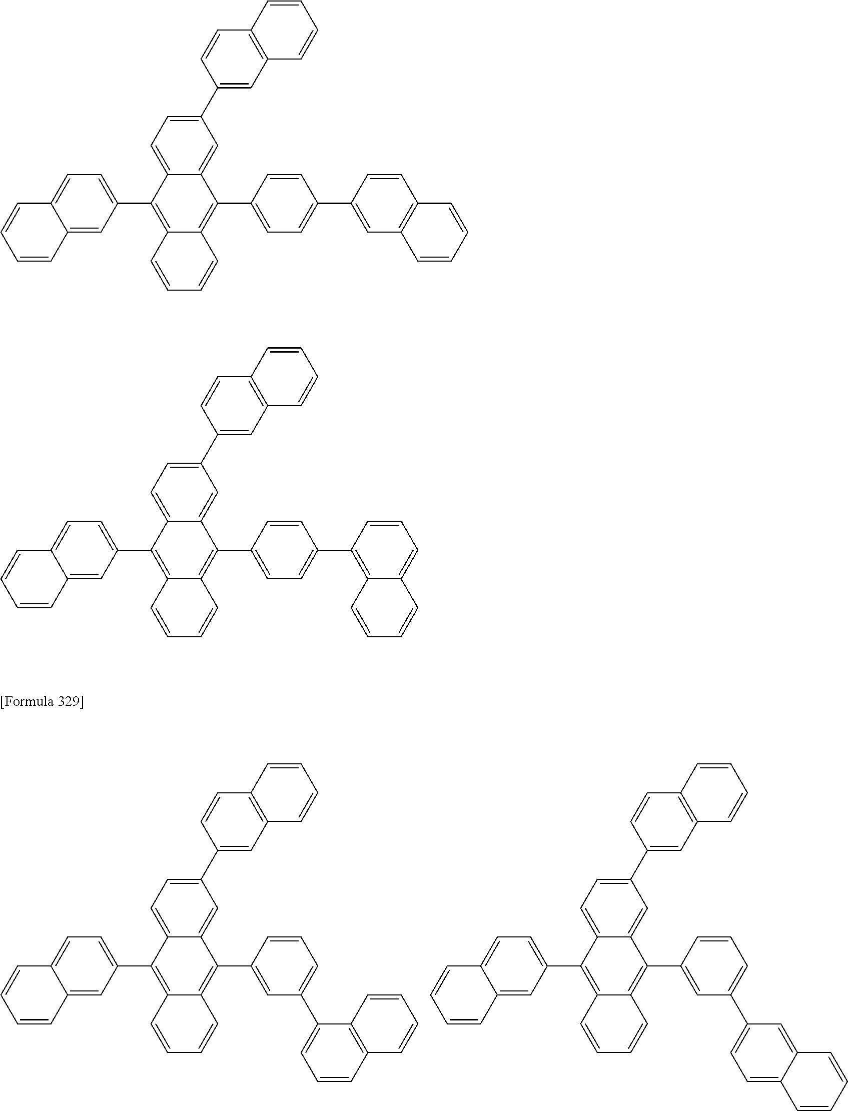

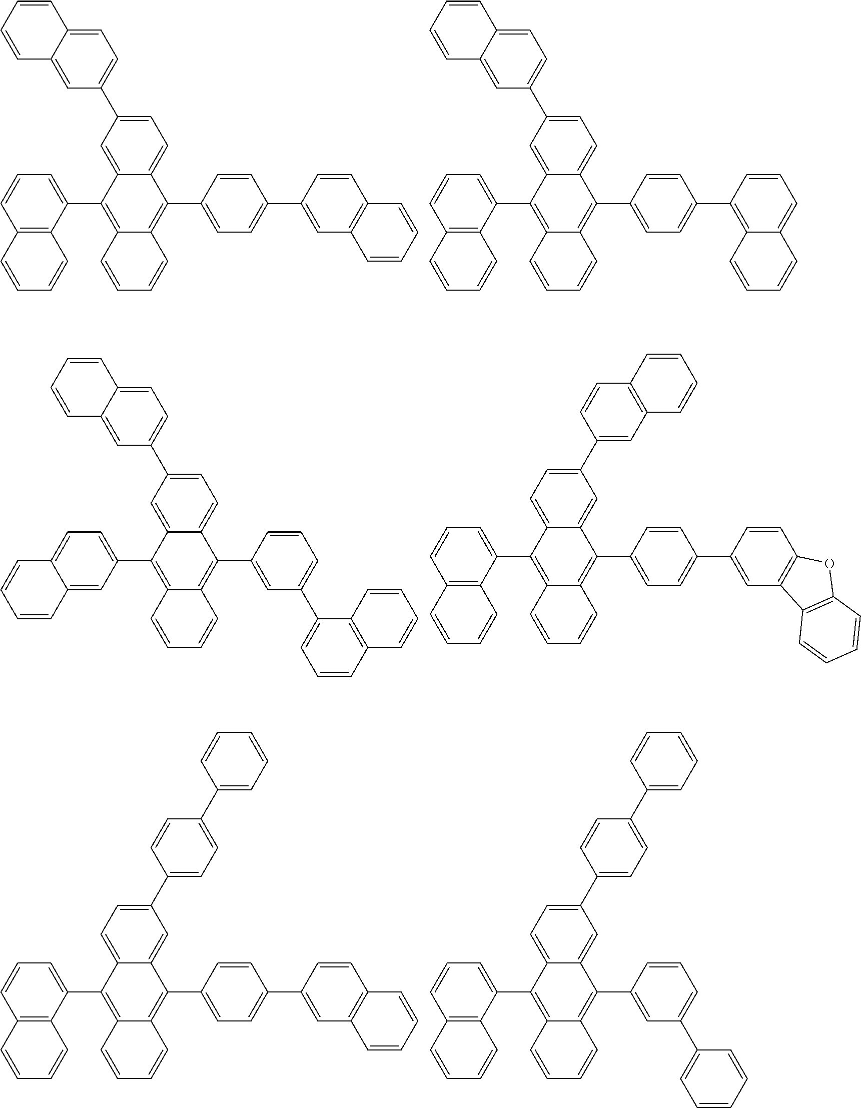

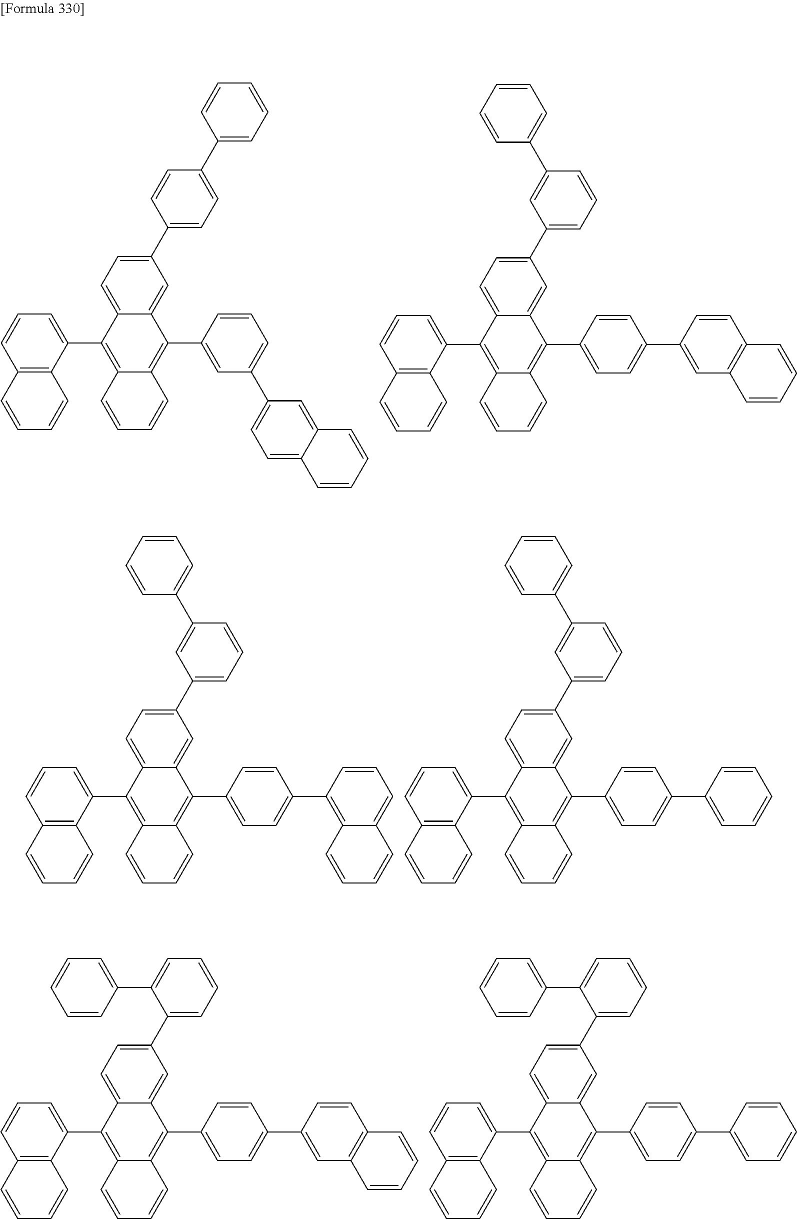

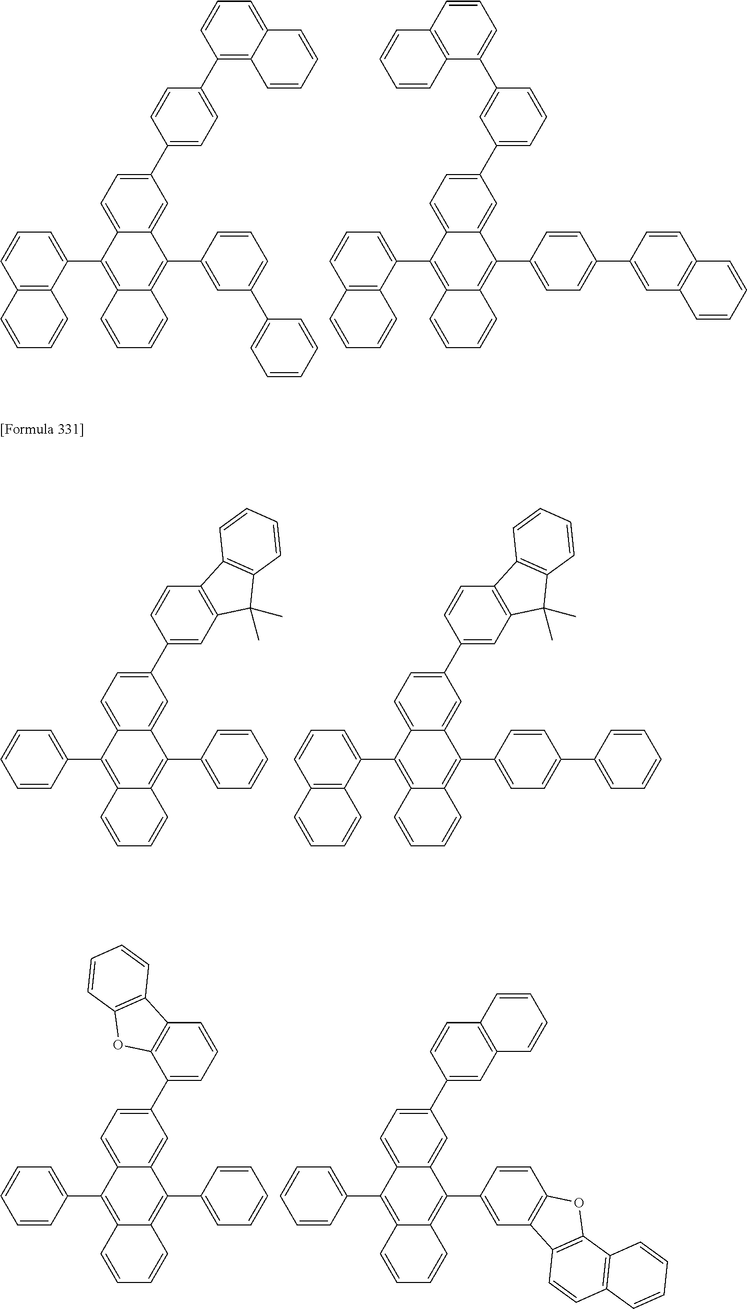

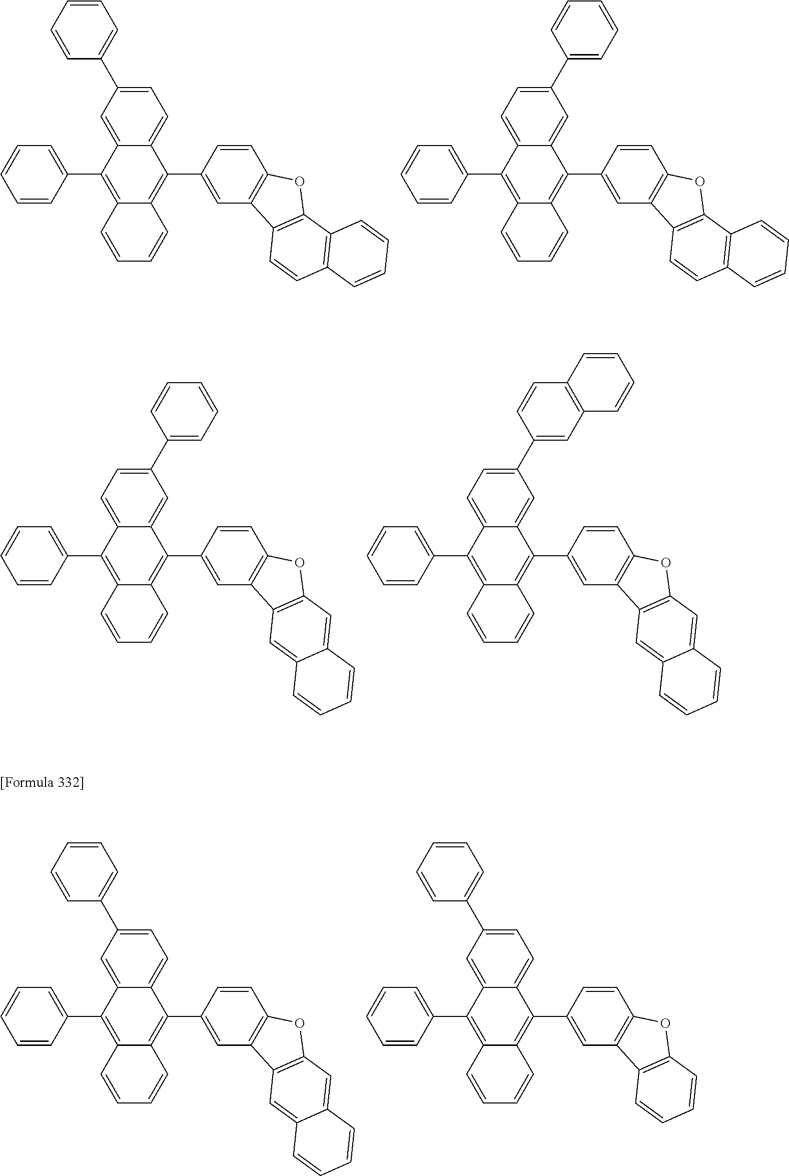 [SMILES: c1ccc(-c2ccc3c(-c4ccc5oc6c7ccccc7ccc6c5c4)c4ccccc4c(-c4ccccc4)c3c2)cc1 ; c1ccc(-c2ccc3c(-c4ccccc4)c4ccccc4c(-c4ccc5oc6c7ccccc7ccc6c5c4)c3c2)cc1 ; c1ccc(-c2ccc3c(-c4ccccc4)c4ccccc4c(-c4ccc5oc6cc7ccccc7cc6c5c4)c3c2)cc1 ; c1ccc(-c2c3ccccc3c(-c3ccc4oc5cc6ccccc6cc5c4c3)c3cc(-c4ccc5ccccc5c4)ccc23)cc1 ; c1ccc(-c2ccc3c(-c4ccc5oc6cc7ccccc7cc6c5c4)c4ccccc4c(-c4ccccc4)c3c2)cc1 ; c1ccc(-c2ccc3c(-c4ccc5oc6ccccc6c5c4)c4ccccc4c(-c4ccccc4)c3c2)cc1]

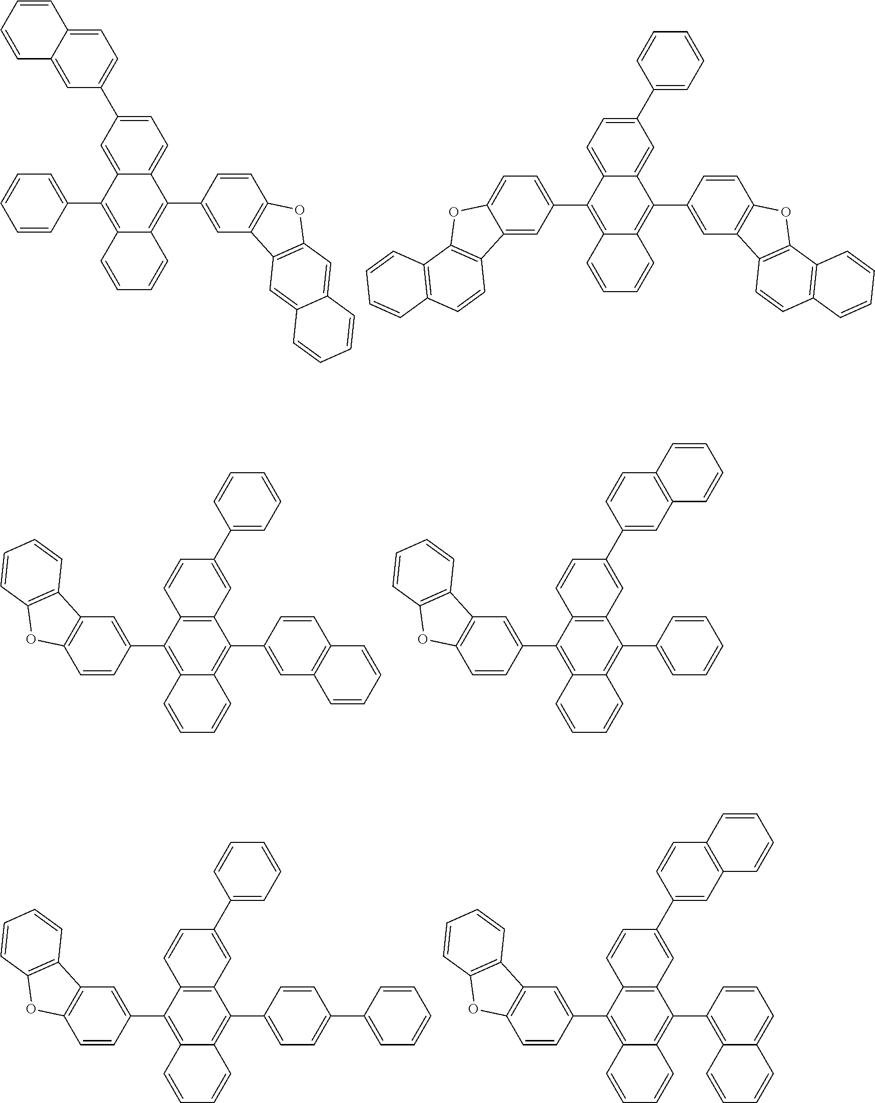

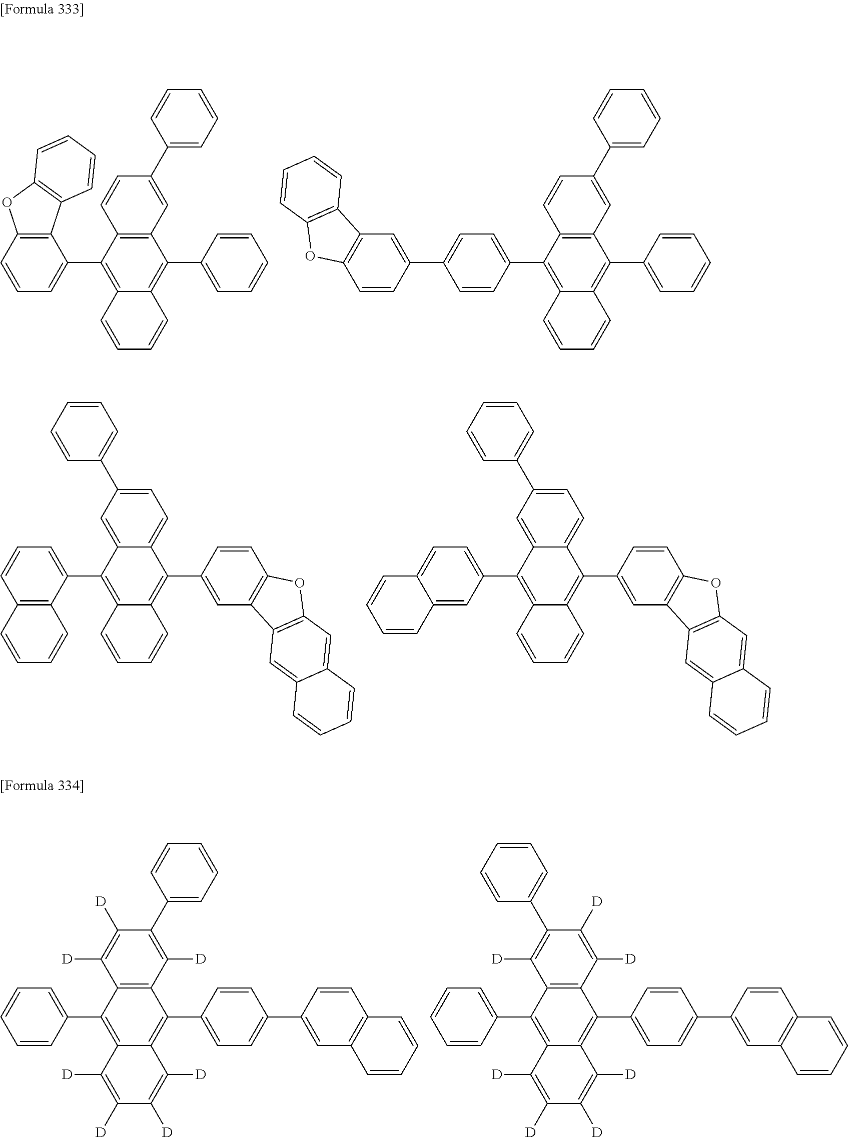

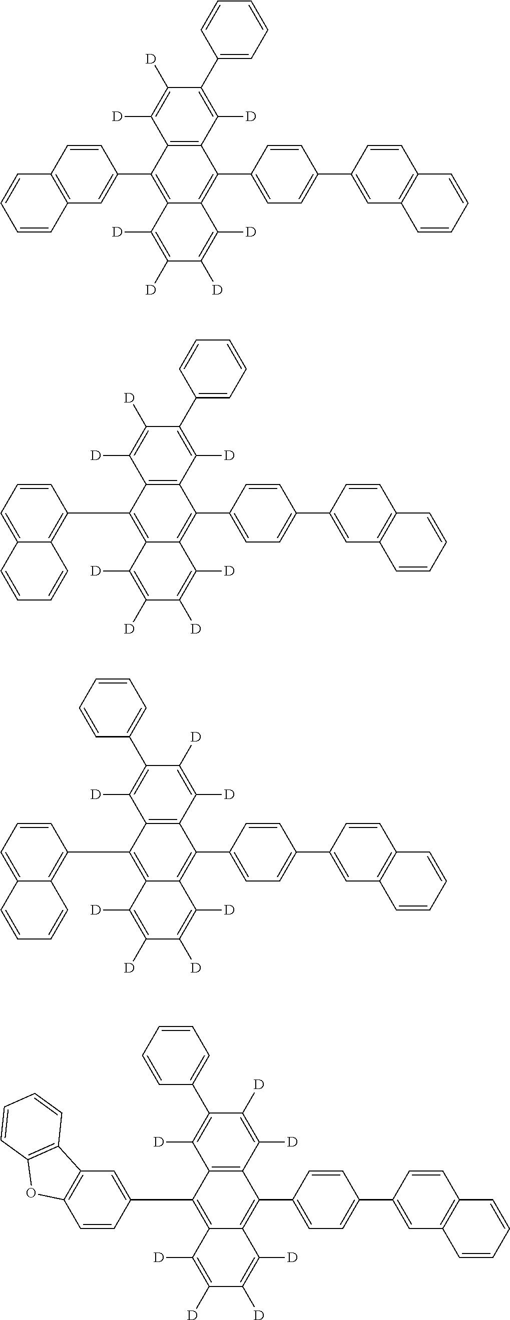 [SMILES: [2H]c1c([2H])c([2H])c2c(-c3ccc(-c4ccc5ccccc5c4)cc3)c3c([2H])c(-c4ccccc4)c([2H])c([2H])c3c(-c3ccc4ccccc4c3)c2c1[2H] ; [2H]c1c([2H])c([2H])c2c(-c3cccc4ccccc34)c3c([2H])c([2H])c(-c4ccccc4)c([2H])c3c(-c3ccc(-c4ccc5ccccc5c4)cc3)c2c1[2H] ; [2H]c1c([2H])c([2H])c2c(-c3cccc4ccccc34)c3c([2H])c(-c4ccccc4)c([2H])c([2H])c3c(-c3ccc(-c4ccc5ccccc5c4)cc3)c2c1[2H] ; [2H]c1c([2H])c([2H])c2c(-c3ccc4oc5ccccc5c4c3)c3c([2H])c(-c4ccccc4)c([2H])c([2H])c3c(-c3ccc(-c4ccc5ccccc5c4)cc3)c2c1[2H]]

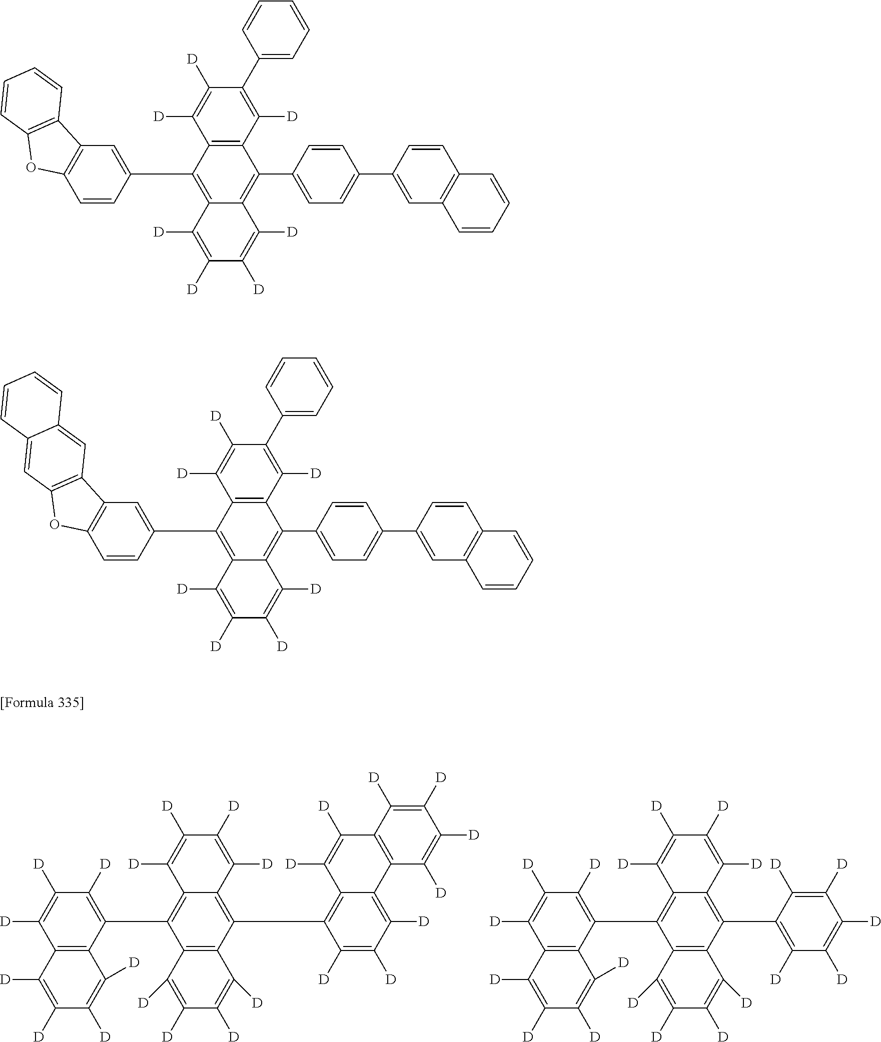

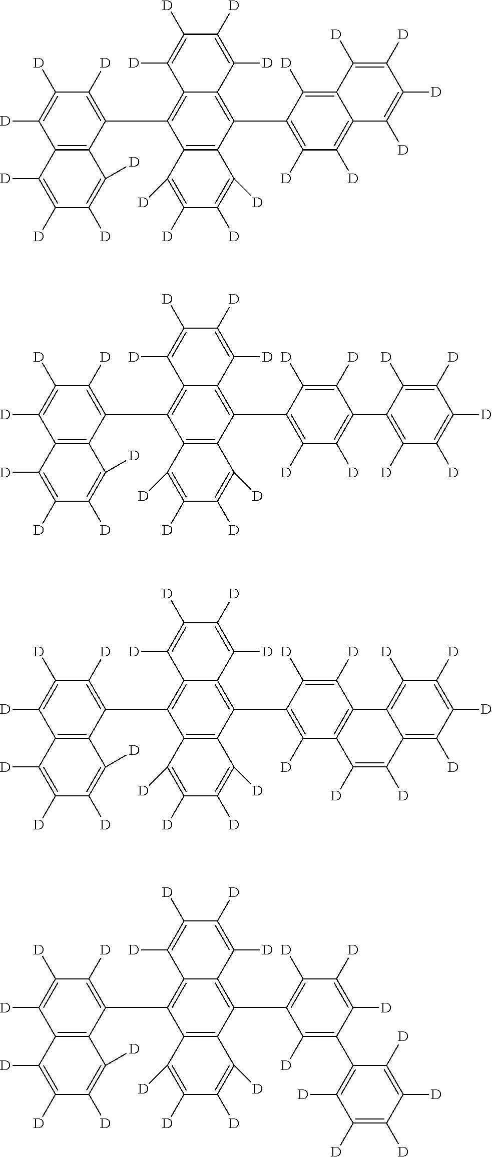

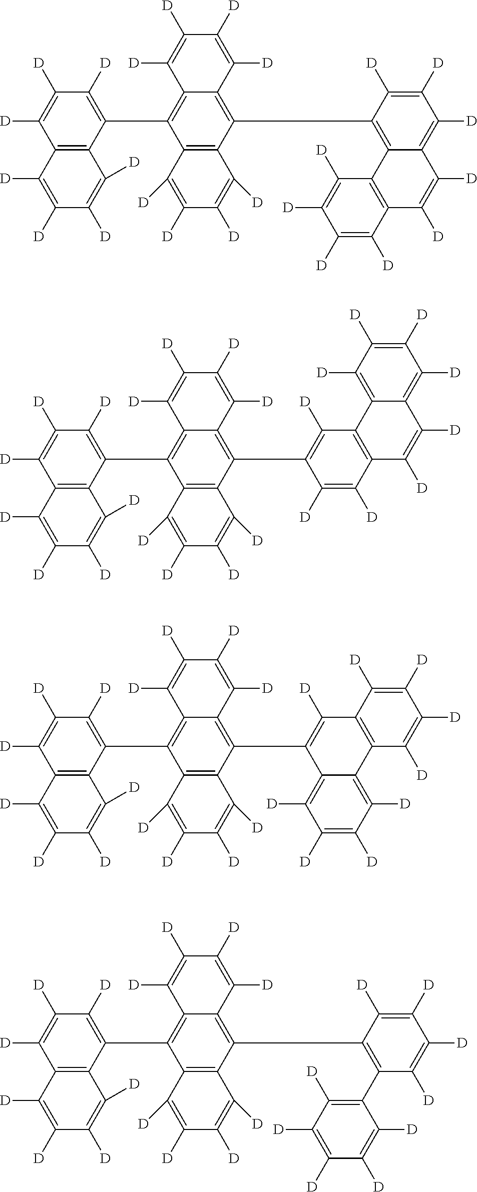

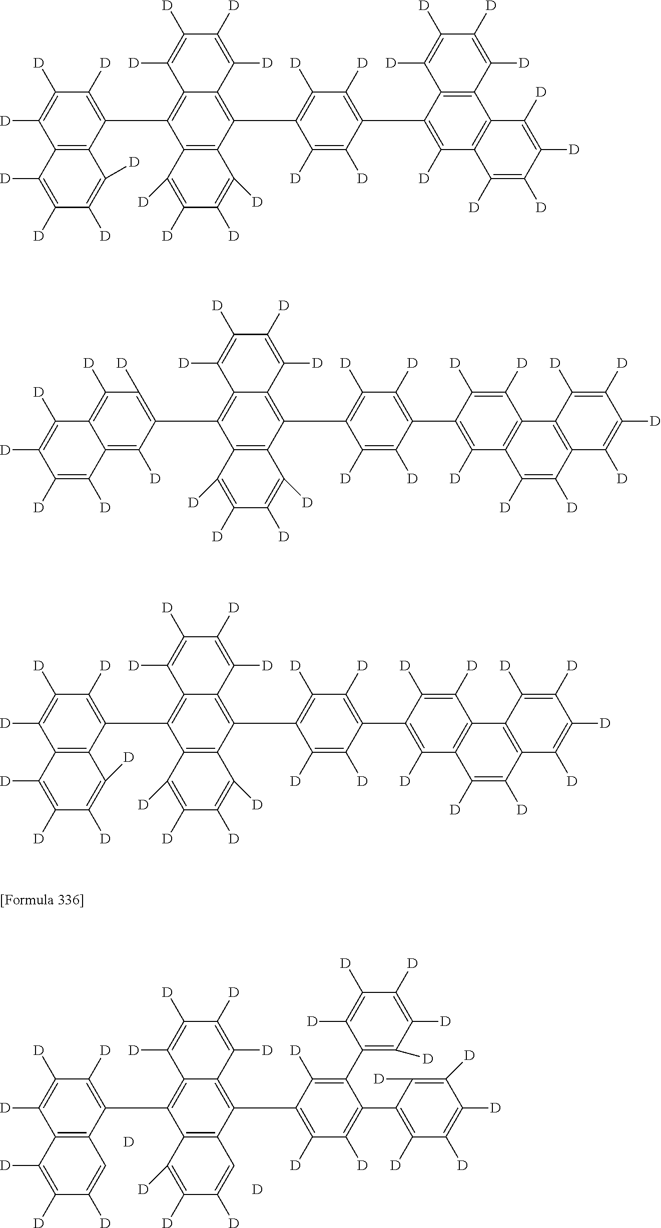

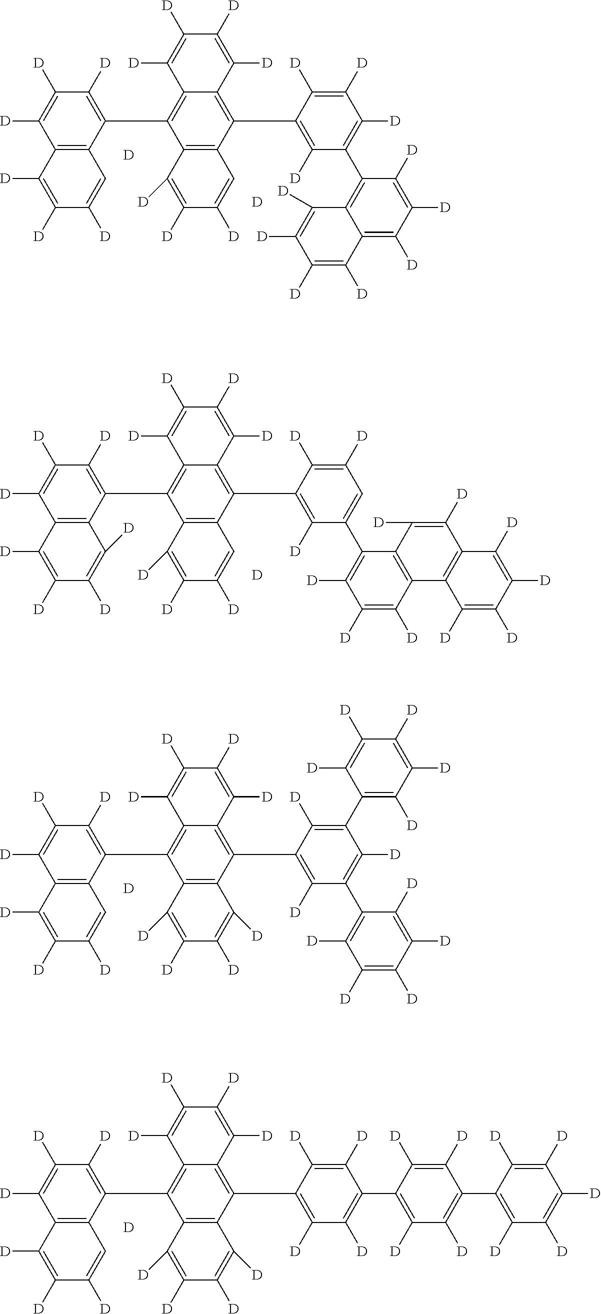

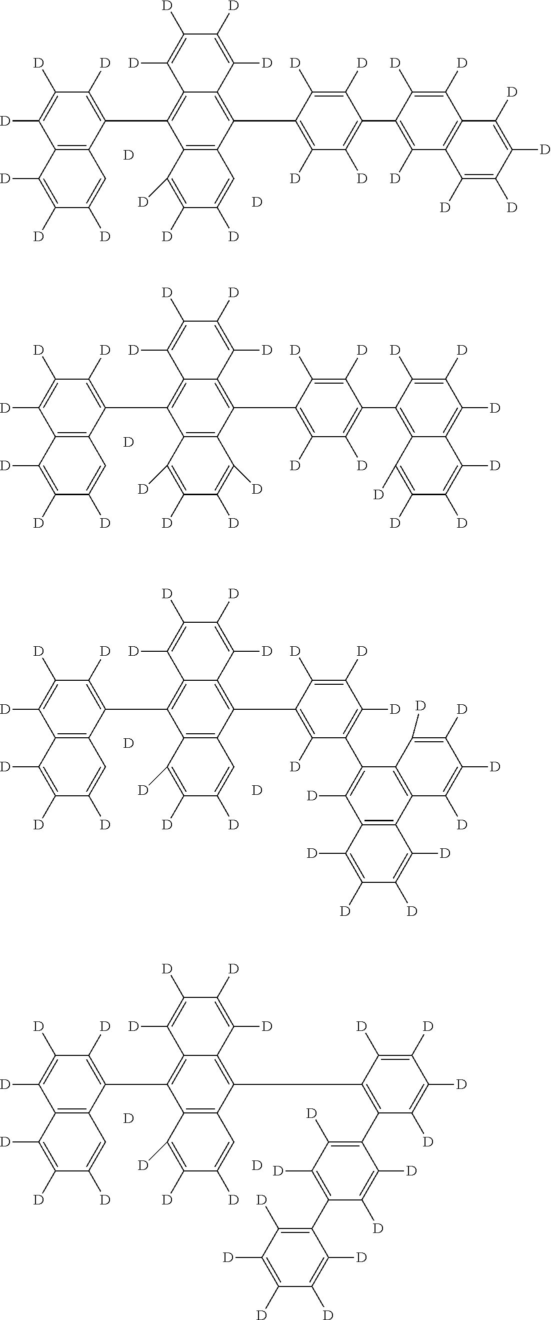

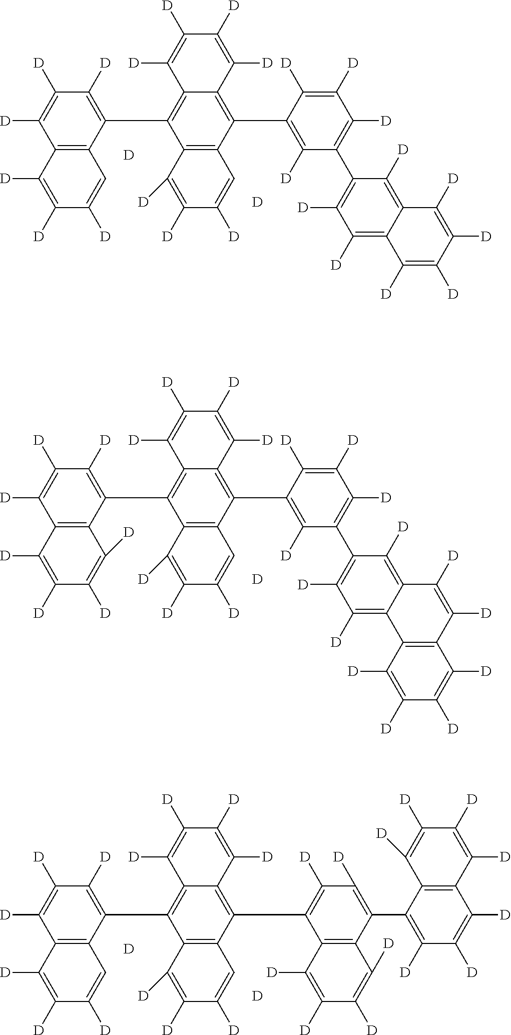

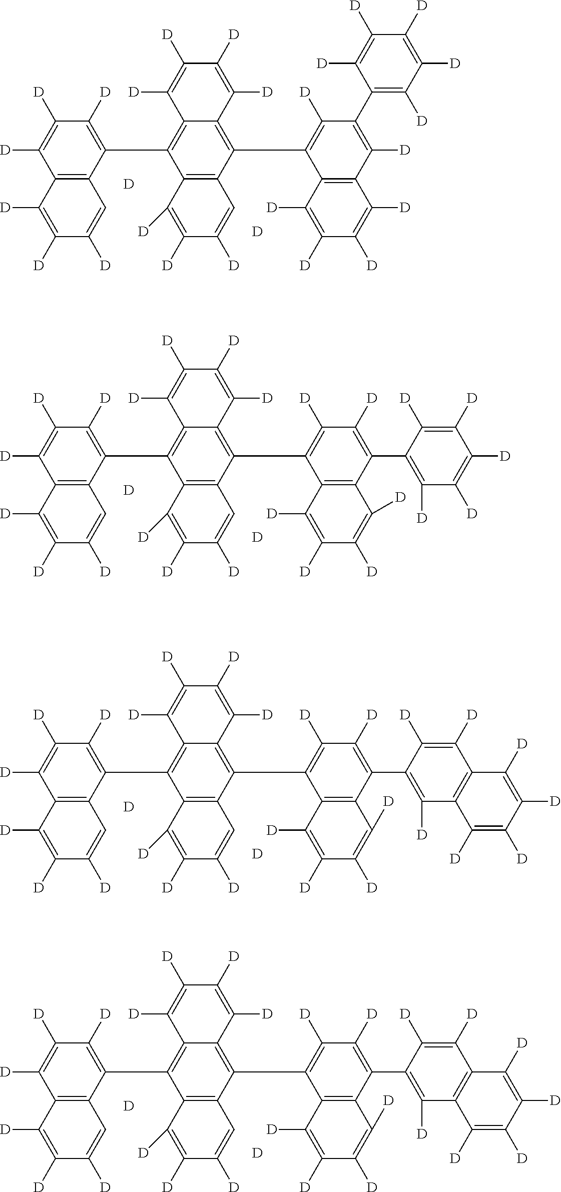

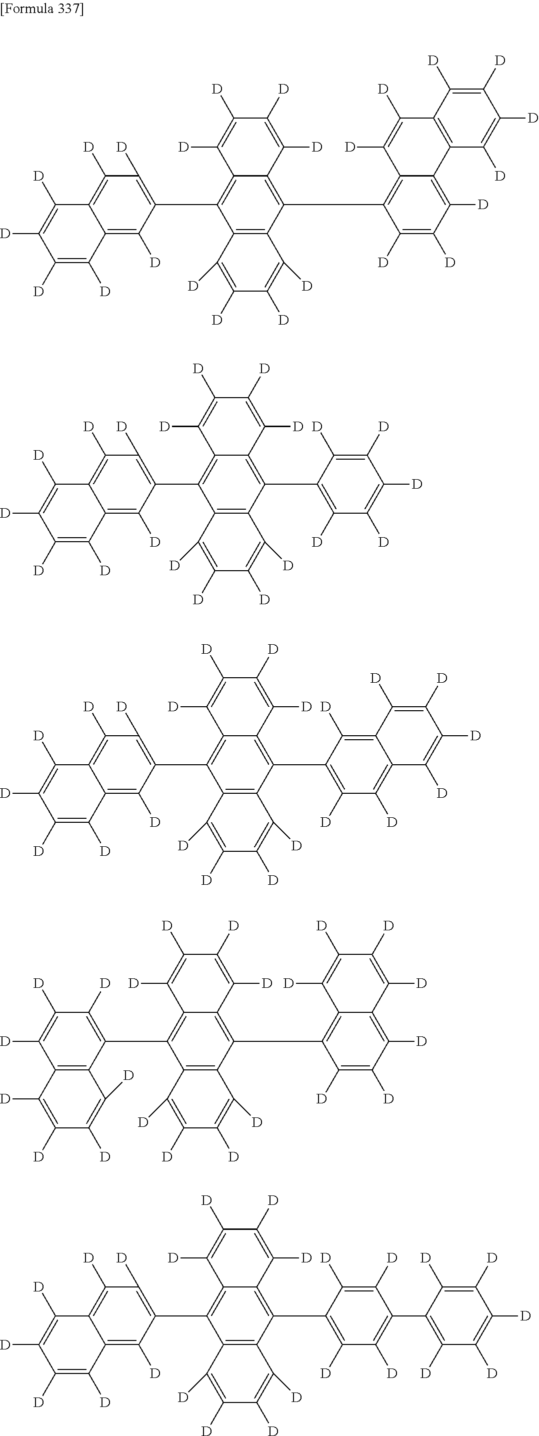

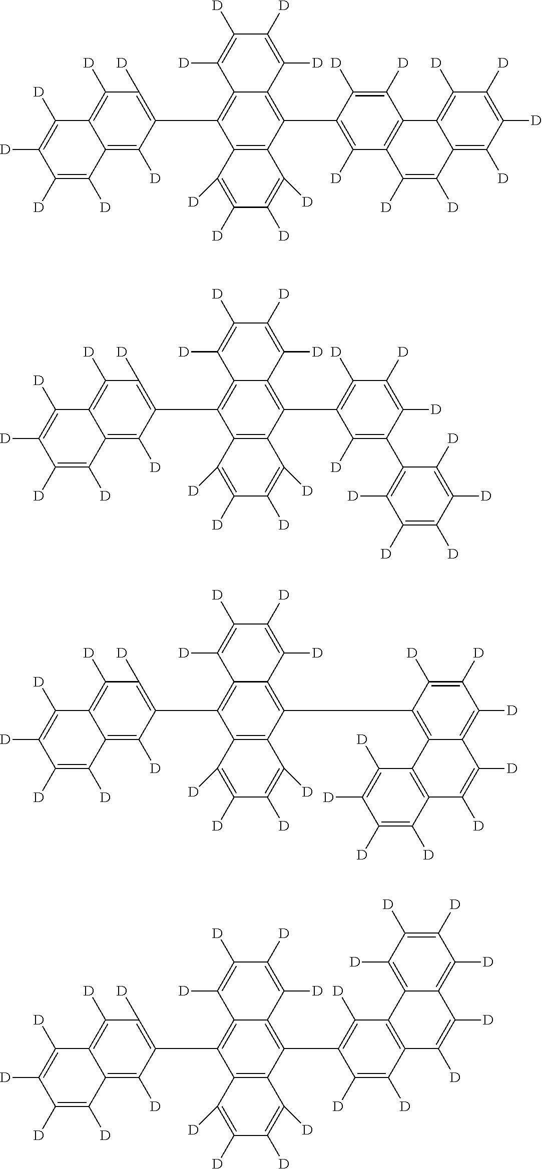

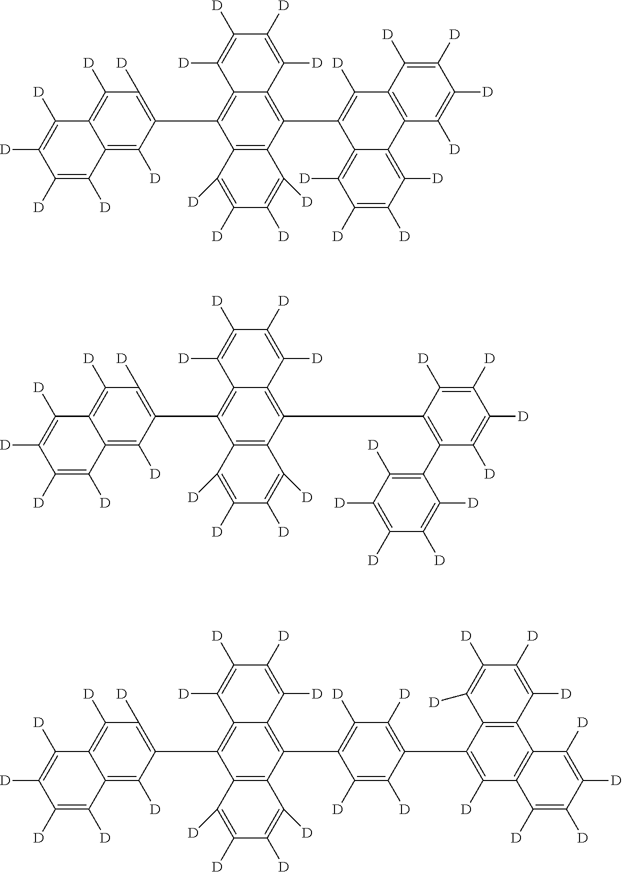

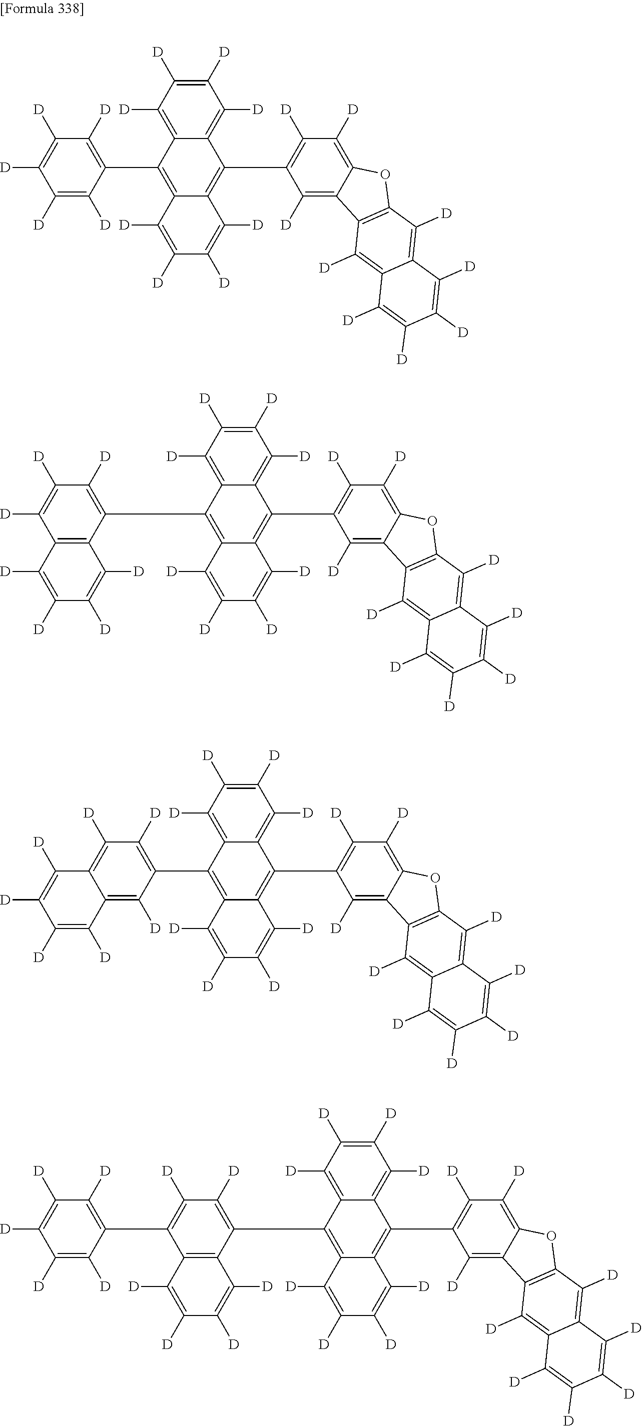

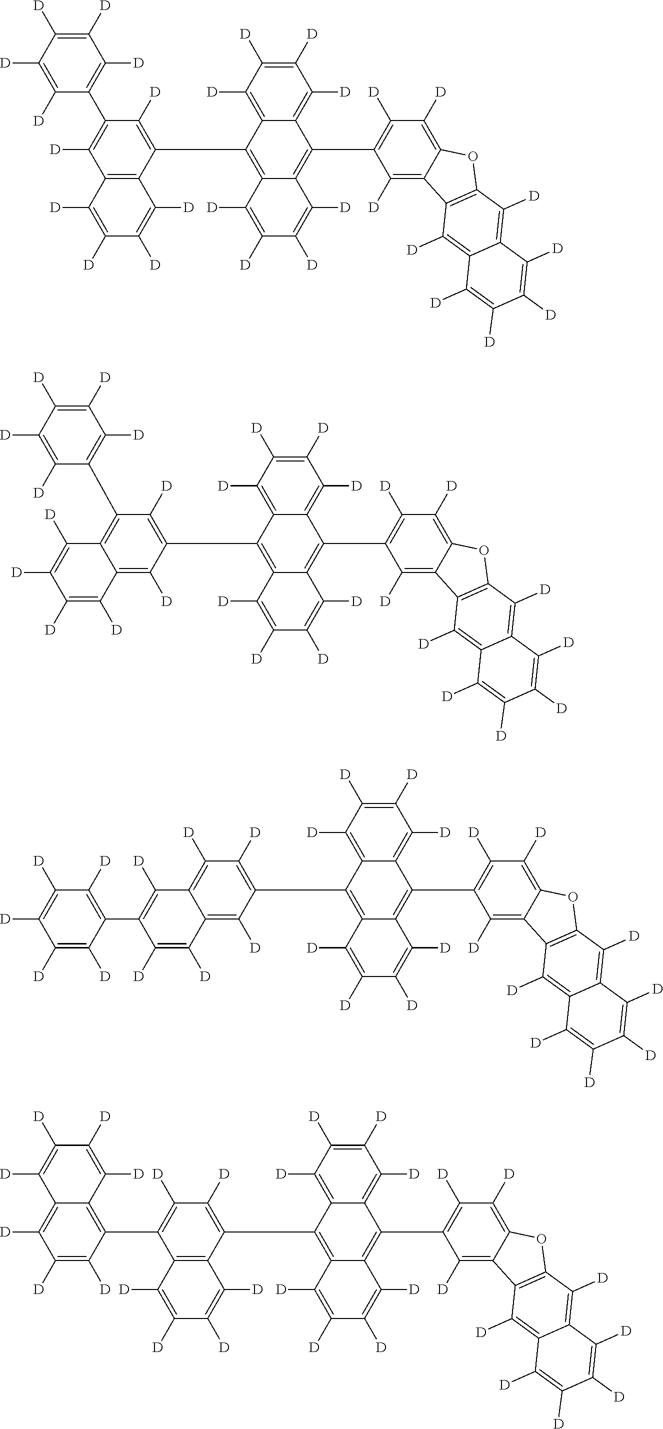

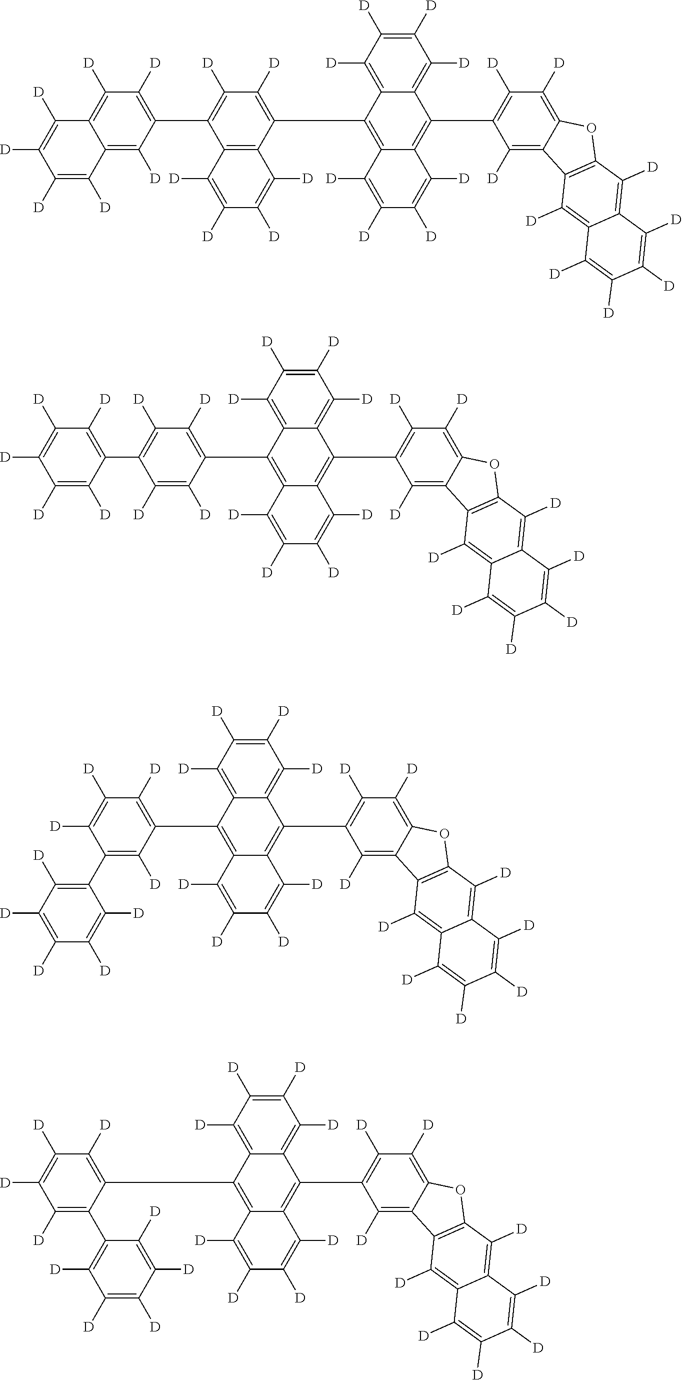

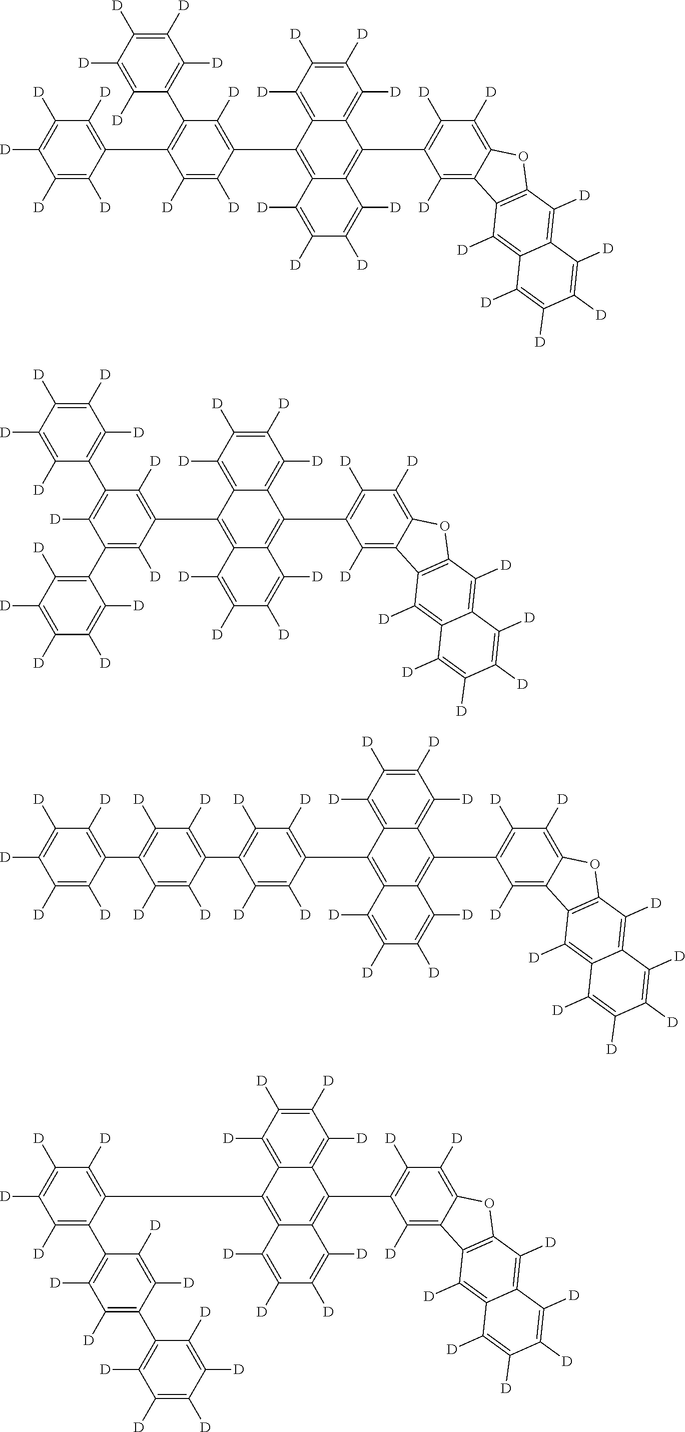

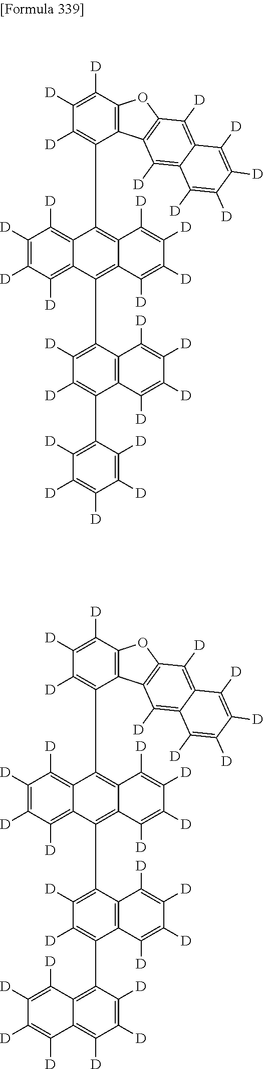

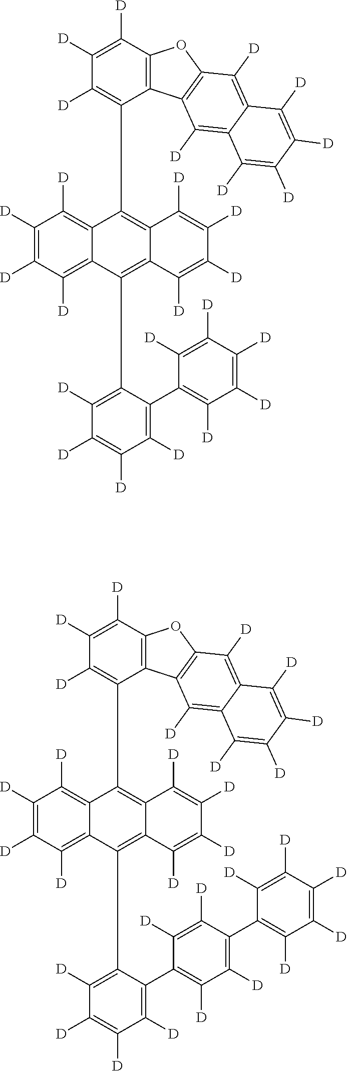 [SMILES: [2H]c1c([2H])c([2H])c(-c2c([2H])c([2H])c([2H])c([2H])c2-c2c3c([2H])c([2H])c([2H])c([2H])c3c(-c3c([2H])c([2H])c([2H])c4oc5c([2H])c6c([2H])c([2H])c([2H])c([2H])c6c([2H])c5c34)c3c([2H])c([2H])c([2H])c([2H])c23)c([2H])c1[2H] ; [2H]c1c([2H])c([2H])c(-c2c([2H])c([2H])c(-c3c([2H])c([2H])c([2H])c([2H])c3-c3c4c([2H])c([2H])c([2H])c([2H])c4c(-c4c([2H])c([2H])c([2H])c5oc6c([2H])c7c([2H])c([2H])c([2H])c([2H])c7c([2H])c6c45)c4c([2H])c([2H])c([2H])c([2H])c34)c([2H])c2[2H])c([2H])c1[2H]]

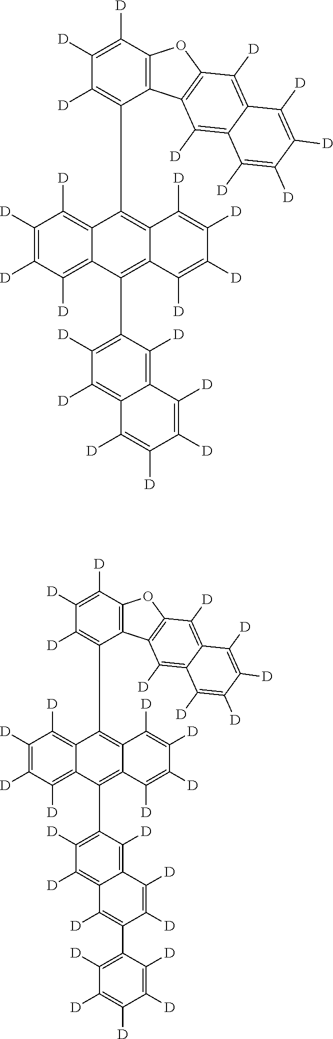 [SMILES: [2H]c1c([2H])c(-c2c3c([2H])c([2H])c([2H])c([2H])c3c(-c3c([2H])c([2H])c4c([2H])c([2H])c([2H])c([2H])c4c3[2H])c3c([2H])c([2H])c([2H])c([2H])c23)c2c(oc3c([2H])c4c([2H])c([2H])c([2H])c([2H])c4c([2H])c32)c1[2H] ; [2H]c1c([2H])c([2H])c(-c2c([2H])c([2H])c3c([2H])c(-c4c5c([2H])c([2H])c([2H])c([2H])c5c(-c5c([2H])c([2H])c([2H])c6oc7c([2H])c8c([2H])c([2H])c([2H])c([2H])c8c([2H])c7c56)c5c([2H])c([2H])c([2H])c([2H])c45)c([2H])c([2H])c3c2[2H])c([2H])c1[2H]]

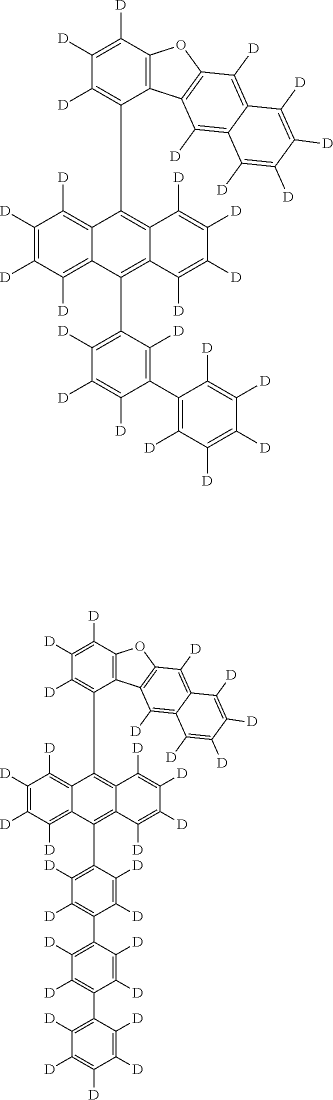 [SMILES: [2H]c1c([2H])c([2H])c(-c2c([2H])c([2H])c([2H])c(-c3c4c([2H])c([2H])c([2H])c([2H])c4c(-c4c([2H])c([2H])c([2H])c5oc6c([2H])c7c([2H])c([2H])c([2H])c([2H])c7c([2H])c6c45)c4c([2H])c([2H])c([2H])c([2H])c34)c2[2H])c([2H])c1[2H] ; [2H]c1c([2H])c([2H])c(-c2c([2H])c([2H])c(-c3c([2H])c([2H])c(-c4c5c([2H])c([2H])c([2H])c([2H])c5c(-c5c([2H])c([2H])c([2H])c6oc7c([2H])c8c([2H])c([2H])c([2H])c([2H])c8c([2H])c7c56)c5c([2H])c([2H])c([2H])c([2H])c45)c([2H])c3[2H])c([2H])c2[2H])c([2H])c1[2H]]

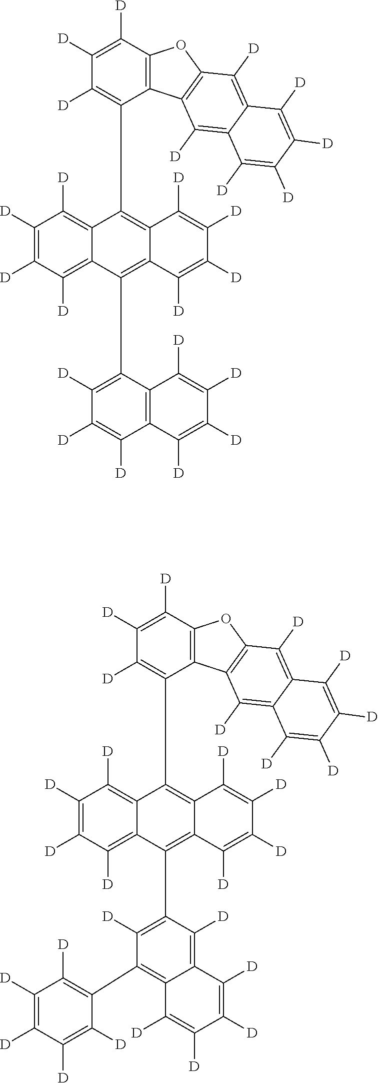 [SMILES: [2H]c1c([2H])c(-c2c3c([2H])c([2H])c([2H])c([2H])c3c(-c3c([2H])c([2H])c([2H])c4c([2H])c([2H])c([2H])c([2H])c34)c3c([2H])c([2H])c([2H])c([2H])c23)c2c(oc3c([2H])c4c([2H])c([2H])c([2H])c([2H])c4c([2H])c32)c1[2H] ; [2H]c1c([2H])c([2H])c(-c2c([2H])c(-c3c4c([2H])c([2H])c([2H])c([2H])c4c(-c4c([2H])c([2H])c([2H])c5oc6c([2H])c7c([2H])c([2H])c([2H])c([2H])c7c([2H])c6c45)c4c([2H])c([2H])c([2H])c([2H])c34)c([2H])c3c([2H])c([2H])c([2H])c([2H])c23)c([2H])c1[2H]]

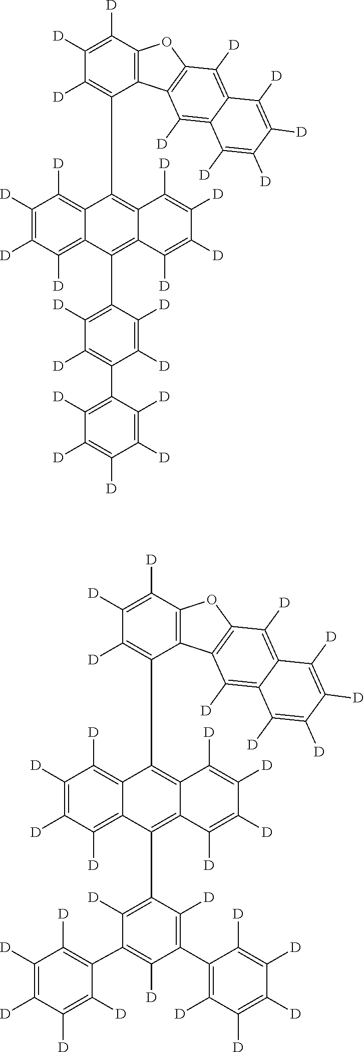 [SMILES: [2H]c1c([2H])c([2H])c(-c2c([2H])c([2H])c(-c3c4c([2H])c([2H])c([2H])c([2H])c4c(-c4c([2H])c([2H])c([2H])c5oc6c([2H])c7c([2H])c([2H])c([2H])c([2H])c7c([2H])c6c45)c4c([2H])c([2H])c([2H])c([2H])c34)c([2H])c2[2H])c([2H])c1[2H] ; [2H]c1c([2H])c([2H])c(-c2c([2H])c(-c3c([2H])c([2H])c([2H])c([2H])c3[2H])c([2H])c(-c3c4c([2H])c([2H])c([2H])c([2H])c4c(-c4c([2H])c([2H])c([2H])c5oc6c([2H])c7c([2H])c([2H])c([2H])c([2H])c7c([2H])c6c45)c4c([2H])c([2H])c([2H])c([2H])c34)c2[2H])c([2H])c1[2H]]

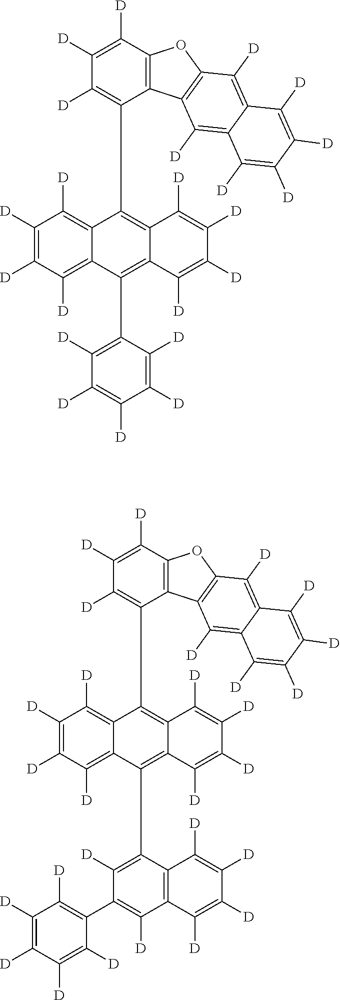 [SMILES: [2H]c1c([2H])c([2H])c(-c2c3c([2H])c([2H])c([2H])c([2H])c3c(-c3c([2H])c([2H])c([2H])c4oc5c([2H])c6c([2H])c([2H])c([2H])c([2H])c6c([2H])c5c34)c3c([2H])c([2H])c([2H])c([2H])c23)c([2H])c1[2H] ; [2H]c1c([2H])c([2H])c(-c2c([2H])c(-c3c4c([2H])c([2H])c([2H])c([2H])c4c(-c4c([2H])c([2H])c([2H])c5oc6c([2H])c7c([2H])c([2H])c([2H])c([2H])c7c([2H])c6c45)c4c([2H])c([2H])c([2H])c([2H])c34)c3c([2H])c([2H])c([2H])c([2H])c3c2[2H])c([2H])c1[2H]]

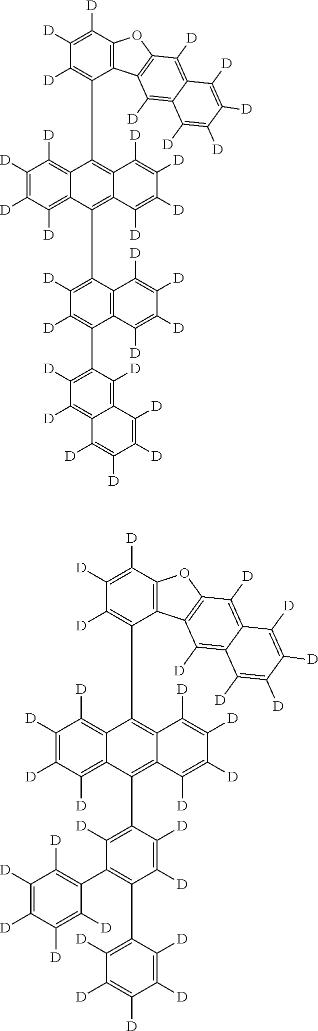

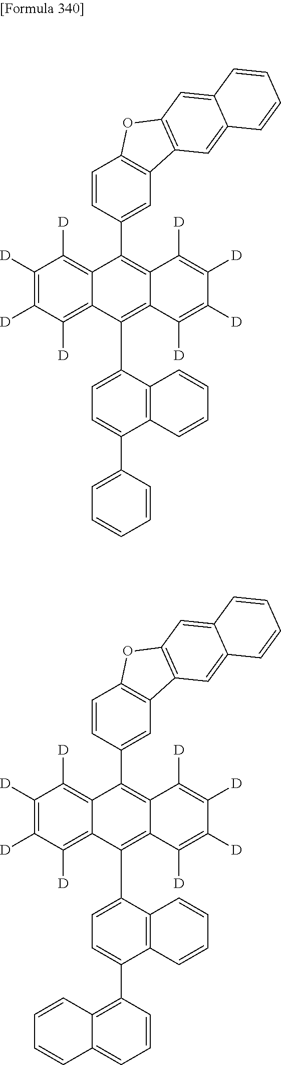 [SMILES: [2H]c1c([2H])c([2H])c2c(-c3ccc(-c4ccccc4)c4ccccc34)c3c([2H])c([2H])c([2H])c([2H])c3c(-c3ccc4oc5cc6ccccc6cc5c4c3)c2c1[2H] ; [2H]c1c([2H])c([2H])c2c(-c3ccc(-c4cccc5ccccc45)c4ccccc34)c3c([2H])c([2H])c([2H])c([2H])c3c(-c3ccc4oc5cc6ccccc6cc5c4c3)c2c1[2H]]

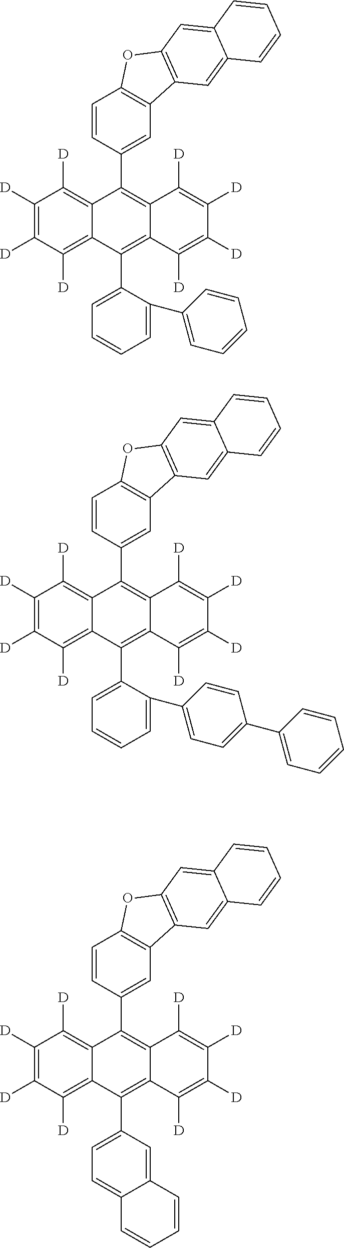

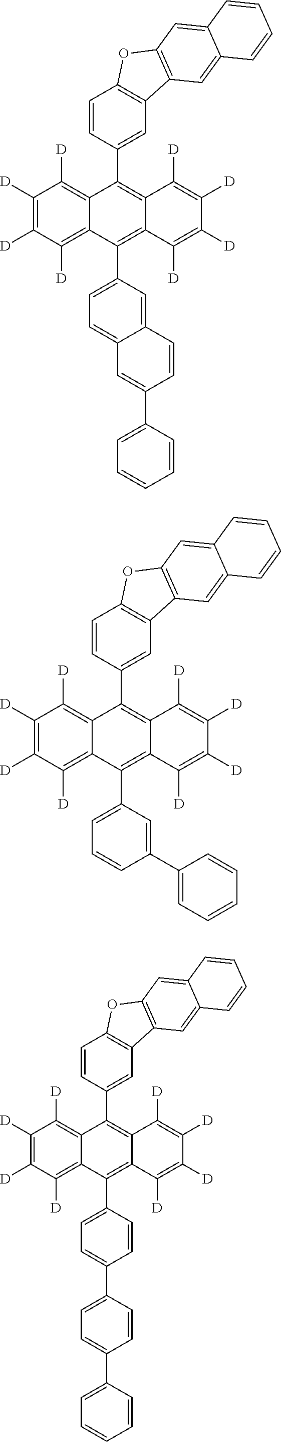

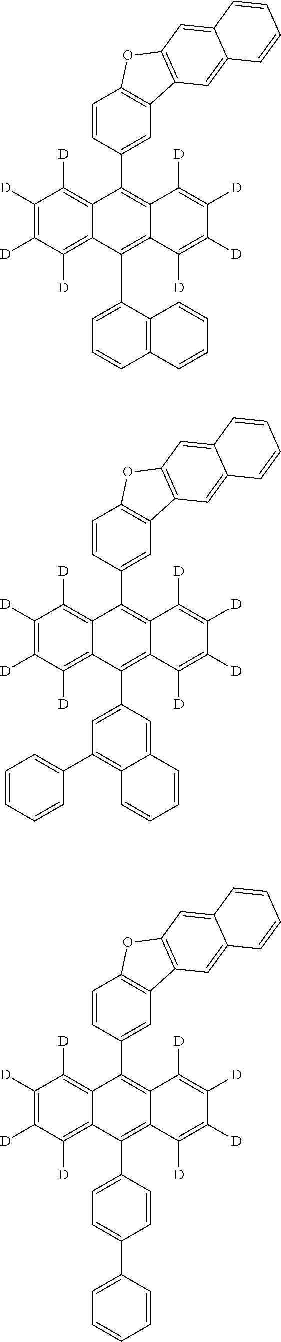 [SMILES: [2H]c1c([2H])c([2H])c2c(-c3cccc4ccccc34)c3c([2H])c([2H])c([2H])c([2H])c3c(-c3ccc4oc5cc6ccccc6cc5c4c3)c2c1[2H] ; [2H]c1c([2H])c([2H])c2c(-c3ccc4oc5cc6ccccc6cc5c4c3)c3c([2H])c([2H])c([2H])c([2H])c3c(-c3cc(-c4ccccc4)c4ccccc4c3)c2c1[2H] ; [2H]c1c([2H])c([2H])c2c(-c3ccc4oc5cc6ccccc6cc5c4c3)c3c([2H])c([2H])c([2H])c([2H])c3c(-c3ccc(-c4ccccc4)cc3)c2c1[2H]]

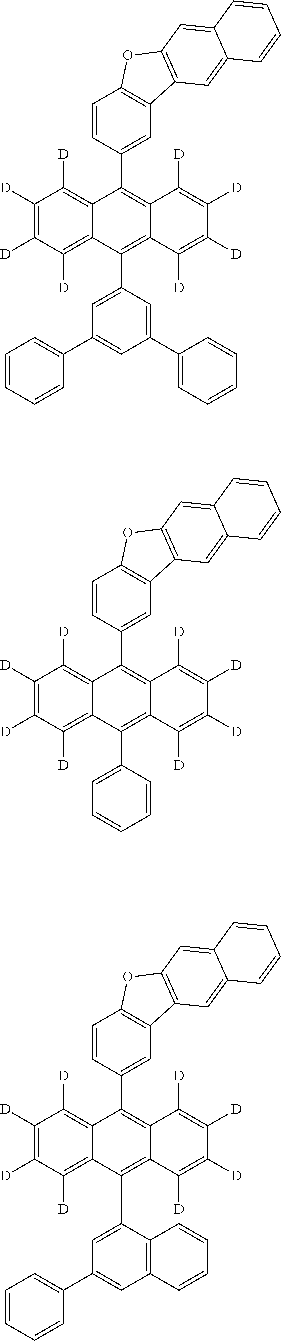 [SMILES: [2H]c1c([2H])c([2H])c2c(-c3ccc4oc5cc6ccccc6cc5c4c3)c3c([2H])c([2H])c([2H])c([2H])c3c(-c3cc(-c4ccccc4)cc(-c4ccccc4)c3)c2c1[2H] ; [2H]c1c([2H])c([2H])c2c(-c3ccc4oc5cc6ccccc6cc5c4c3)c3c([2H])c([2H])c([2H])c([2H])c3c(-c3ccccc3)c2c1[2H] ; [2H]c1c([2H])c([2H])c2c(-c3cc(-c4ccccc4)cc4ccccc34)c3c([2H])c([2H])c([2H])c([2H])c3c(-c3ccc4oc5cc6ccccc6cc5c4c3)c2c1[2H]]

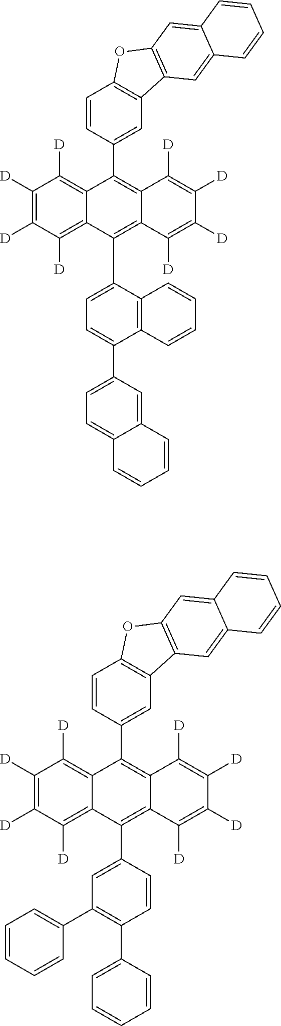 [SMILES: [2H]c1c([2H])c([2H])c2c(-c3ccc(-c4ccc5ccccc5c4)c4ccccc34)c3c([2H])c([2H])c([2H])c([2H])c3c(-c3ccc4oc5cc6ccccc6cc5c4c3)c2c1[2H] ; [2H]c1c([2H])c([2H])c2c(-c3ccc4oc5cc6ccccc6cc5c4c3)c3c([2H])c([2H])c([2H])c([2H])c3c(-c3ccc(-c4ccccc4)c(-c4ccccc4)c3)c2c1[2H]]

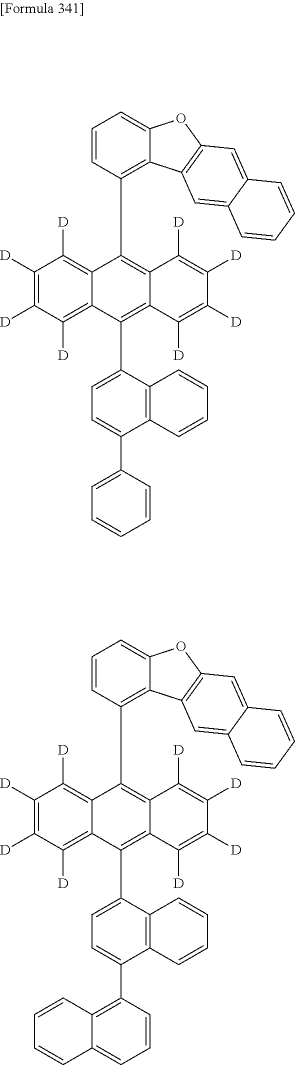 [SMILES: [2H]c1c([2H])c([2H])c2c(-c3cccc4oc5cc6ccccc6cc5c34)c3c([2H])c([2H])c([2H])c([2H])c3c(-c3ccc(-c4ccccc4)c4ccccc34)c2c1[2H] ; [2H]c1c([2H])c([2H])c2c(-c3cccc4oc5cc6ccccc6cc5c34)c3c([2H])c([2H])c([2H])c([2H])c3c(-c3ccc(-c4cccc5ccccc45)c4ccccc34)c2c1[2H]]

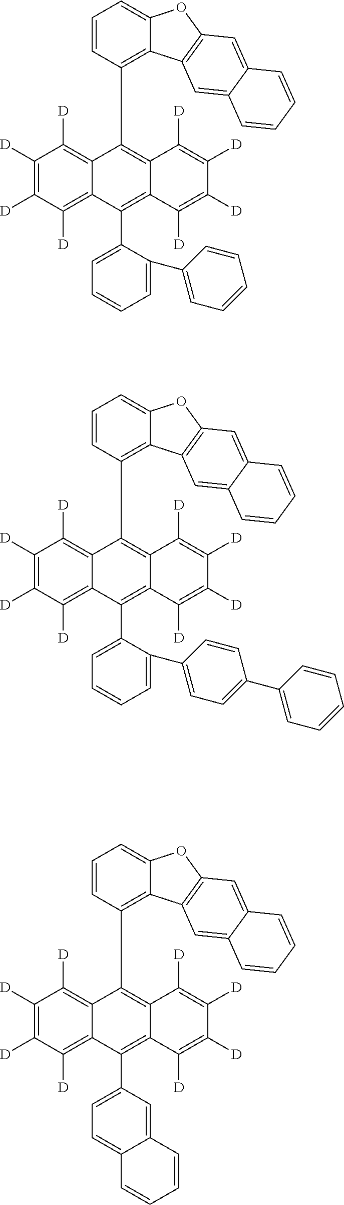 [SMILES: [2H]c1c([2H])c([2H])c2c(-c3cccc4oc5cc6ccccc6cc5c34)c3c([2H])c([2H])c([2H])c([2H])c3c(-c3ccccc3-c3ccccc3)c2c1[2H] ; [2H]c1c([2H])c([2H])c2c(-c3cccc4oc5cc6ccccc6cc5c34)c3c([2H])c([2H])c([2H])c([2H])c3c(-c3ccccc3-c3ccc(-c4ccccc4)cc3)c2c1[2H] ; [2H]c1c([2H])c([2H])c2c(-c3cccc4oc5cc6ccccc6cc5c34)c3c([2H])c([2H])c([2H])c([2H])c3c(-c3ccc4ccccc4c3)c2c1[2H]]

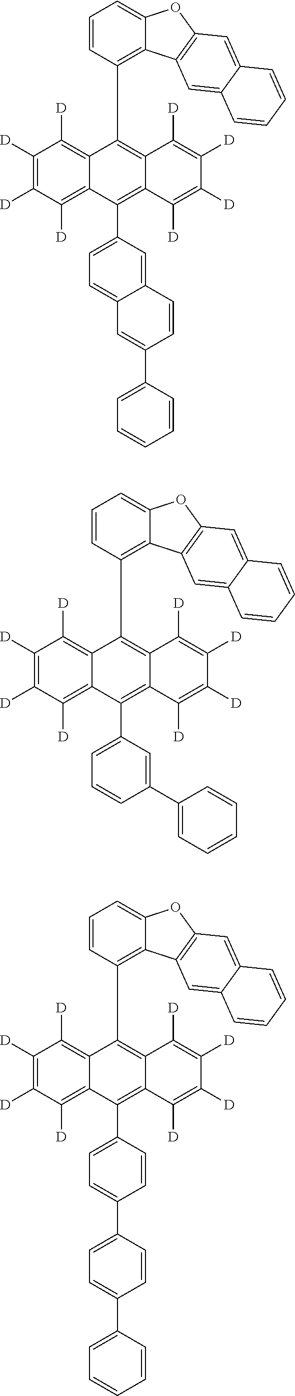 [SMILES: [2H]c1c([2H])c([2H])c2c(-c3cccc4oc5cc6ccccc6cc5c34)c3c([2H])c([2H])c([2H])c([2H])c3c(-c3ccc4cc(-c5ccccc5)ccc4c3)c2c1[2H] ; [2H]c1c([2H])c([2H])c2c(-c3cccc4oc5cc6ccccc6cc5c34)c3c([2H])c([2H])c([2H])c([2H])c3c(-c3cccc(-c4ccccc4)c3)c2c1[2H] ; [2H]c1c([2H])c([2H])c2c(-c3cccc4oc5cc6ccccc6cc5c34)c3c([2H])c([2H])c([2H])c([2H])c3c(-c3ccc(-c4ccc(-c5ccccc5)cc4)cc3)c2c1[2H]]

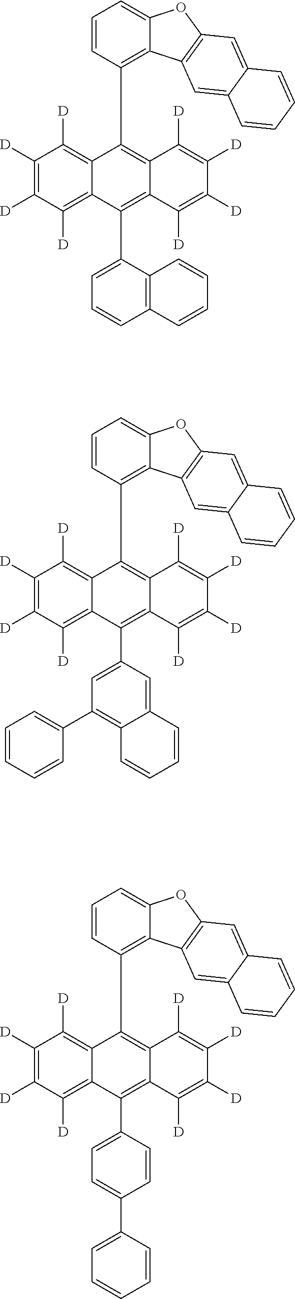 [SMILES: [2H]c1c([2H])c([2H])c2c(-c3cccc4oc5cc6ccccc6cc5c34)c3c([2H])c([2H])c([2H])c([2H])c3c(-c3cccc4ccccc34)c2c1[2H] ; [2H]c1c([2H])c([2H])c2c(-c3cccc4oc5cc6ccccc6cc5c34)c3c([2H])c([2H])c([2H])c([2H])c3c(-c3cc(-c4ccccc4)c4ccccc4c3)c2c1[2H] ; [2H]c1c([2H])c([2H])c2c(-c3cccc4oc5cc6ccccc6cc5c34)c3c([2H])c([2H])c([2H])c([2H])c3c(-c3ccc(-c4ccccc4)cc3)c2c1[2H]]

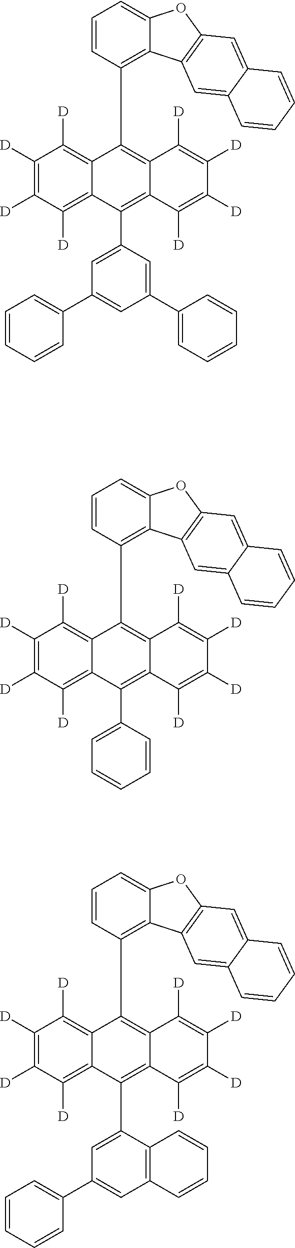 [SMILES: [2H]c1c([2H])c([2H])c2c(-c3cccc4oc5cc6ccccc6cc5c34)c3c([2H])c([2H])c([2H])c([2H])c3c(-c3cc(-c4ccccc4)cc(-c4ccccc4)c3)c2c1[2H] ; [2H]c1c([2H])c([2H])c2c(-c3cccc4oc5cc6ccccc6cc5c34)c3c([2H])c([2H])c([2H])c([2H])c3c(-c3ccccc3)c2c1[2H] ; [2H]c1c([2H])c([2H])c2c(-c3cccc4oc5cc6ccccc6cc5c34)c3c([2H])c([2H])c([2H])c([2H])c3c(-c3cc(-c4ccccc4)cc4ccccc34)c2c1[2H]]

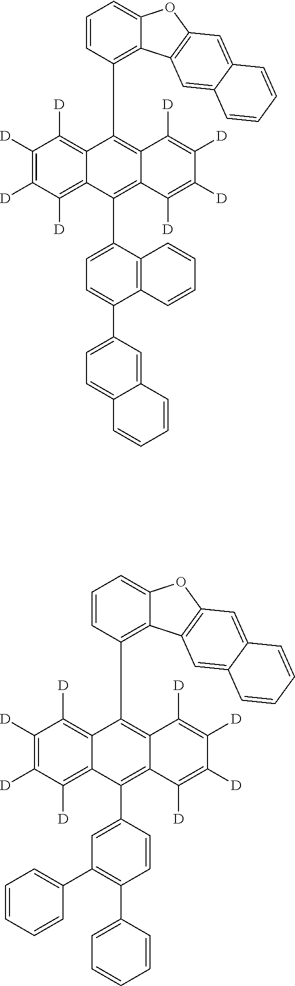 [SMILES: [2H]c1c([2H])c([2H])c2c(-c3cccc4oc5cc6ccccc6cc5c34)c3c([2H])c([2H])c([2H])c([2H])c3c(-c3ccc(-c4ccc5ccccc5c4)c4ccccc34)c2c1[2H] ; [2H]c1c([2H])c([2H])c2c(-c3cccc4oc5cc6ccccc6cc5c34)c3c([2H])c([2H])c([2H])c([2H])c3c(-c3ccc(-c4ccccc4)c(-c4ccccc4)c3)c2c1[2H]]

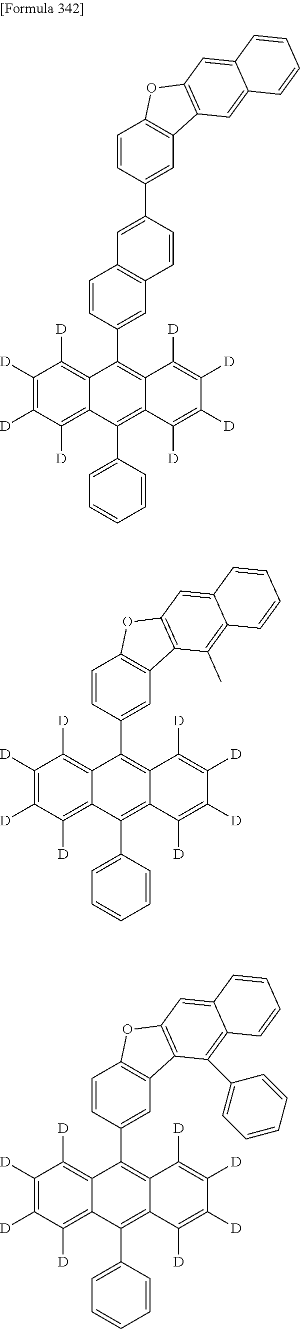 [SMILES: [2H]c1c([2H])c([2H])c2c(-c3ccc4cc(-c5ccc6oc7cc8ccccc8cc7c6c5)ccc4c3)c3c([2H])c([2H])c([2H])c([2H])c3c(-c3ccccc3)c2c1[2H] ; [2H]c1c([2H])c([2H])c2c(-c3ccc4oc5cc6ccccc6c(C)c5c4c3)c3c([2H])c([2H])c([2H])c([2H])c3c(-c3ccccc3)c2c1[2H] ; [2H]c1c([2H])c([2H])c2c(-c3ccc4oc5cc6ccccc6c(-c6ccccc6)c5c4c3)c3c([2H])c([2H])c([2H])c([2H])c3c(-c3ccccc3)c2c1[2H]]

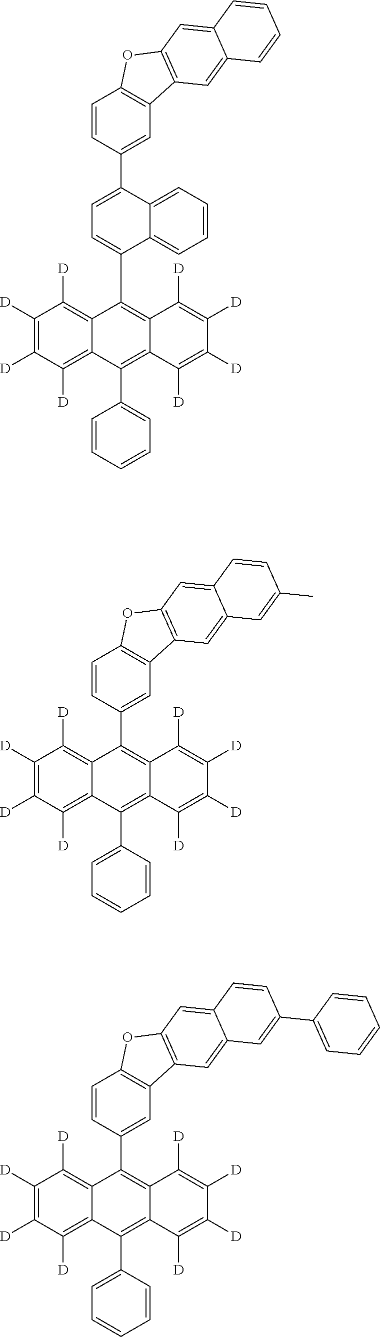 [SMILES: [2H]c1c([2H])c([2H])c2c(-c3ccc(-c4ccc5oc6cc7ccccc7cc6c5c4)c4ccccc34)c3c([2H])c([2H])c([2H])c([2H])c3c(-c3ccccc3)c2c1[2H] ; [2H]c1c([2H])c([2H])c2c(-c3ccc4oc5cc6ccc(C)cc6cc5c4c3)c3c([2H])c([2H])c([2H])c([2H])c3c(-c3ccccc3)c2c1[2H] ; [2H]c1c([2H])c([2H])c2c(-c3ccc4oc5cc6ccc(-c7ccccc7)cc6cc5c4c3)c3c([2H])c([2H])c([2H])c([2H])c3c(-c3ccccc3)c2c1[2H]]

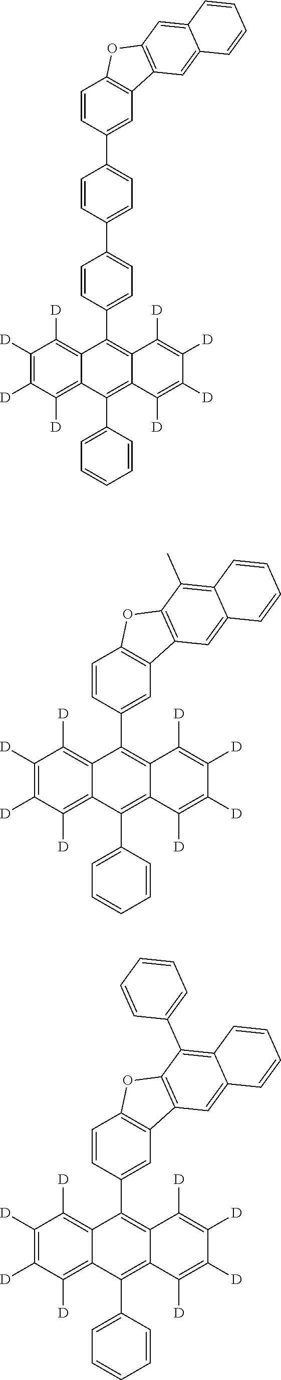 [SMILES: [2H]c1c([2H])c([2H])c2c(-c3ccc(-c4ccc(-c5ccc6oc7cc8ccccc8cc7c6c5)cc4)cc3)c3c([2H])c([2H])c([2H])c([2H])c3c(-c3ccccc3)c2c1[2H] ; [2H]c1c([2H])c([2H])c2c(-c3ccc4oc5c(C)c6ccccc6cc5c4c3)c3c([2H])c([2H])c([2H])c([2H])c3c(-c3ccccc3)c2c1[2H] ; [2H]c1c([2H])c([2H])c2c(-c3ccc4oc5c(-c6ccccc6)c6ccccc6cc5c4c3)c3c([2H])c([2H])c([2H])c([2H])c3c(-c3ccccc3)c2c1[2H]]

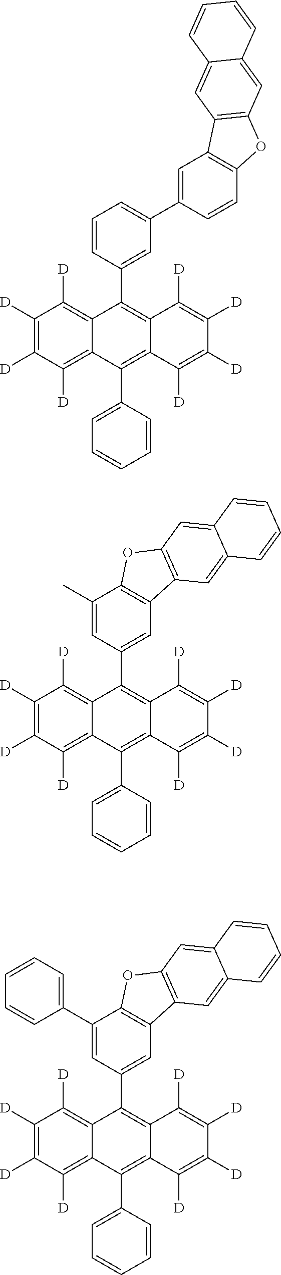 [SMILES: [2H]c1c([2H])c([2H])c2c(-c3cccc(-c4ccc5oc6cc7ccccc7cc6c5c4)c3)c3c([2H])c([2H])c([2H])c([2H])c3c(-c3ccccc3)c2c1[2H] ; [2H]c1c([2H])c([2H])c2c(-c3cc(C)c4oc5cc6ccccc6cc5c4c3)c3c([2H])c([2H])c([2H])c([2H])c3c(-c3ccccc3)c2c1[2H] ; [2H]c1c([2H])c([2H])c2c(-c3cc(-c4ccccc4)c4oc5cc6ccccc6cc5c4c3)c3c([2H])c([2H])c([2H])c([2H])c3c(-c3ccccc3)c2c1[2H]]

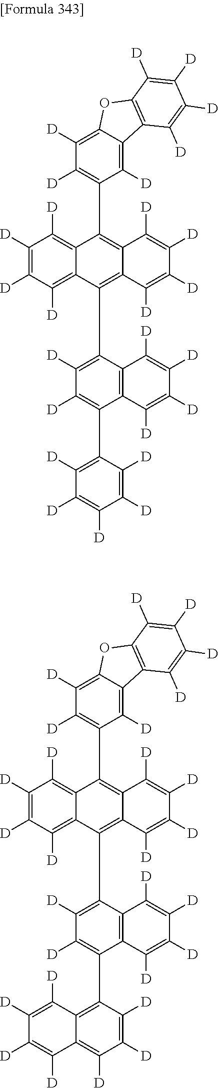 [SMILES: [2H]c1c([2H])c([2H])c(-c2c([2H])c([2H])c(-c3c4c([2H])c([2H])c([2H])c([2H])c4c(-c4c([2H])c([2H])c5oc6c([2H])c([2H])c([2H])c([2H])c6c5c4[2H])c4c([2H])c([2H])c([2H])c([2H])c34)c3c([2H])c([2H])c([2H])c([2H])c23)c([2H])c1[2H] ; [2H]c1c([2H])c([2H])c2c(oc3c([2H])c([2H])c(-c4c5c([2H])c([2H])c([2H])c([2H])c5c(-c5c([2H])c([2H])c(-c6c([2H])c([2H])c([2H])c7c([2H])c([2H])c([2H])c([2H])c67)c6c([2H])c([2H])c([2H])c([2H])c56)c5c([2H])c([2H])c([2H])c([2H])c45)c([2H])c32)c1[2H]]

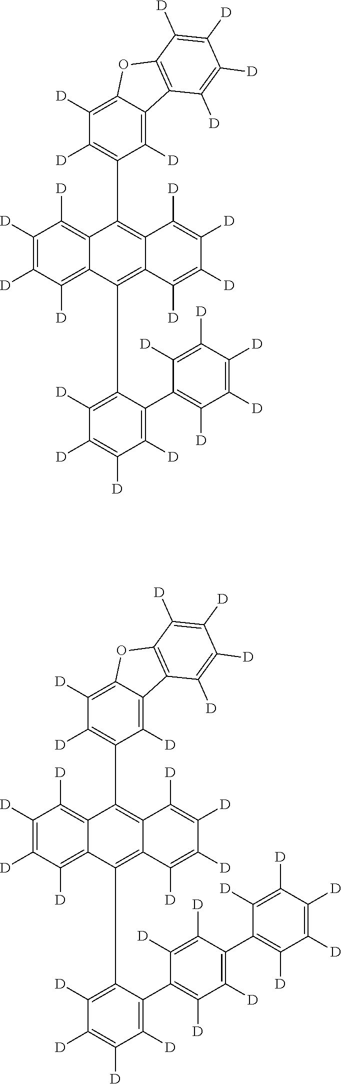 [SMILES: [2H]c1c([2H])c([2H])c(-c2c([2H])c([2H])c([2H])c([2H])c2-c2c3c([2H])c([2H])c([2H])c([2H])c3c(-c3c([2H])c([2H])c4oc5c([2H])c([2H])c([2H])c([2H])c5c4c3[2H])c3c([2H])c([2H])c([2H])c([2H])c23)c([2H])c1[2H] ; [2H]c1c([2H])c([2H])c(-c2c([2H])c([2H])c(-c3c([2H])c([2H])c([2H])c([2H])c3-c3c4c([2H])c([2H])c([2H])c([2H])c4c(-c4c([2H])c([2H])c5oc6c([2H])c([2H])c([2H])c([2H])c6c5c4[2H])c4c([2H])c([2H])c([2H])c([2H])c34)c([2H])c2[2H])c([2H])c1[2H]]

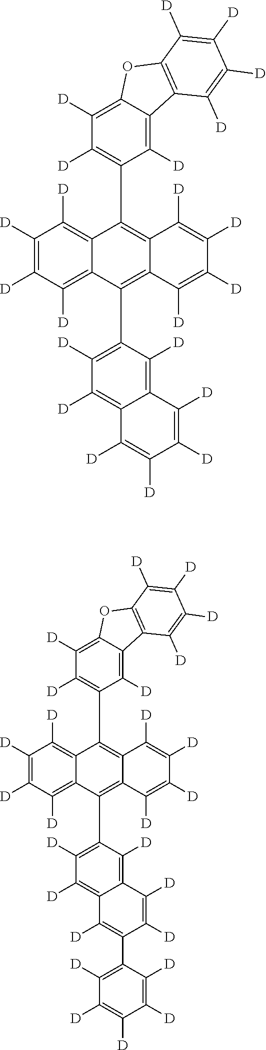 [SMILES: [2H]c1c([2H])c([2H])c2c(oc3c([2H])c([2H])c(-c4c5c([2H])c([2H])c([2H])c([2H])c5c(-c5c([2H])c([2H])c6c([2H])c([2H])c([2H])c([2H])c6c5[2H])c5c([2H])c([2H])c([2H])c([2H])c45)c([2H])c32)c1[2H] ; [2H]c1c([2H])c([2H])c(-c2c([2H])c([2H])c3c([2H])c(-c4c5c([2H])c([2H])c([2H])c([2H])c5c(-c5c([2H])c([2H])c6oc7c([2H])c([2H])c([2H])c([2H])c7c6c5[2H])c5c([2H])c([2H])c([2H])c([2H])c45)c([2H])c([2H])c3c2[2H])c([2H])c1[2H]]

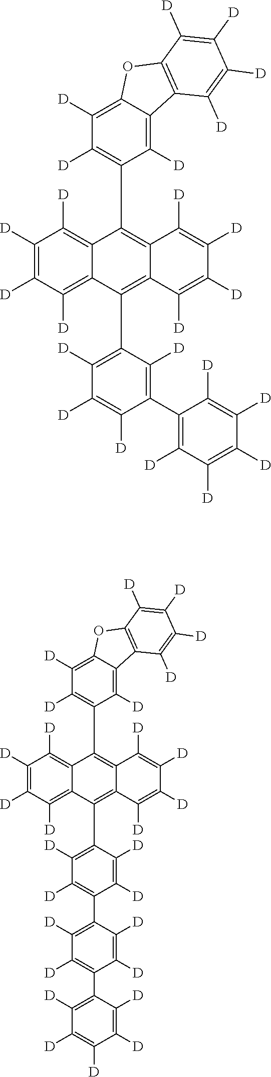 [SMILES: [2H]c1c([2H])c([2H])c(-c2c([2H])c([2H])c([2H])c(-c3c4c([2H])c([2H])c([2H])c([2H])c4c(-c4c([2H])c([2H])c5oc6c([2H])c([2H])c([2H])c([2H])c6c5c4[2H])c4c([2H])c([2H])c([2H])c([2H])c34)c2[2H])c([2H])c1[2H] ; [2H]c1c([2H])c([2H])c(-c2c([2H])c([2H])c(-c3c([2H])c([2H])c(-c4c5c([2H])c([2H])c([2H])c([2H])c5c(-c5c([2H])c([2H])c6oc7c([2H])c([2H])c([2H])c([2H])c7c6c5[2H])c5c([2H])c([2H])c([2H])c([2H])c45)c([2H])c3[2H])c([2H])c2[2H])c([2H])c1[2H]]

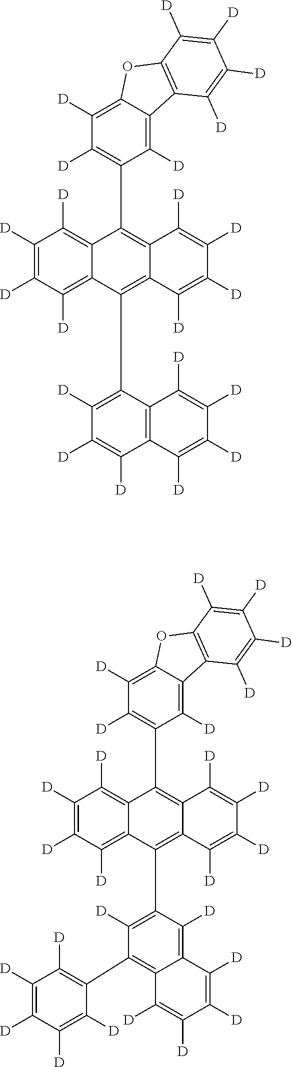 [SMILES: [2H]c1c([2H])c([2H])c2c(oc3c([2H])c([2H])c(-c4c5c([2H])c([2H])c([2H])c([2H])c5c(-c5c([2H])c([2H])c([2H])c6c([2H])c([2H])c([2H])c([2H])c56)c5c([2H])c([2H])c([2H])c([2H])c45)c([2H])c32)c1[2H] ; [2H]c1c([2H])c([2H])c(-c2c([2H])c(-c3c4c([2H])c([2H])c([2H])c([2H])c4c(-c4c([2H])c([2H])c5oc6c([2H])c([2H])c([2H])c([2H])c6c5c4[2H])c4c([2H])c([2H])c([2H])c([2H])c34)c([2H])c3c([2H])c([2H])c([2H])c([2H])c23)c([2H])c1[2H]]

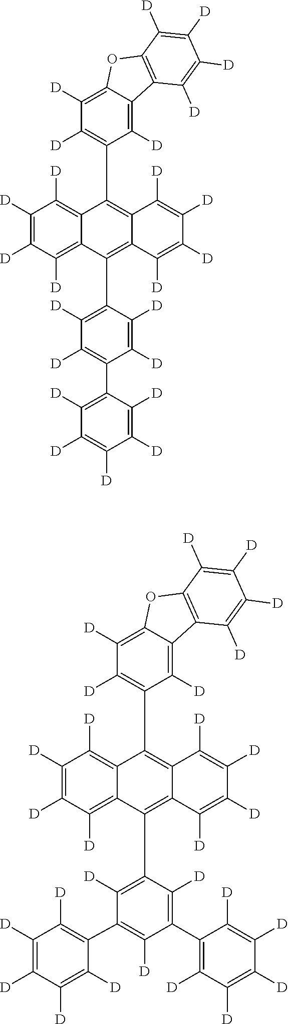 [SMILES: [2H]c1c([2H])c([2H])c(-c2c([2H])c([2H])c(-c3c4c([2H])c([2H])c([2H])c([2H])c4c(-c4c([2H])c([2H])c5oc6c([2H])c([2H])c([2H])c([2H])c6c5c4[2H])c4c([2H])c([2H])c([2H])c([2H])c34)c([2H])c2[2H])c([2H])c1[2H] ; [2H]c1c([2H])c([2H])c(-c2c([2H])c(-c3c([2H])c([2H])c([2H])c([2H])c3[2H])c([2H])c(-c3c4c([2H])c([2H])c([2H])c([2H])c4c(-c4c([2H])c([2H])c5oc6c([2H])c([2H])c([2H])c([2H])c6c5c4[2H])c4c([2H])c([2H])c([2H])c([2H])c34)c2[2H])c([2H])c1[2H]]

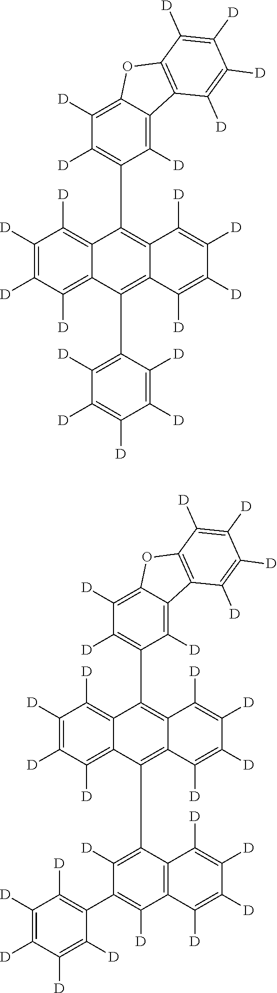 [SMILES: [2H]c1c([2H])c([2H])c(-c2c3c([2H])c([2H])c([2H])c([2H])c3c(-c3c([2H])c([2H])c4oc5c([2H])c([2H])c([2H])c([2H])c5c4c3[2H])c3c([2H])c([2H])c([2H])c([2H])c23)c([2H])c1[2H] ; [2H]c1c([2H])c([2H])c(-c2c([2H])c(-c3c4c([2H])c([2H])c([2H])c([2H])c4c(-c4c([2H])c([2H])c5oc6c([2H])c([2H])c([2H])c([2H])c6c5c4[2H])c4c([2H])c([2H])c([2H])c([2H])c34)c3c([2H])c([2H])c([2H])c([2H])c3c2[2H])c([2H])c1[2H]]

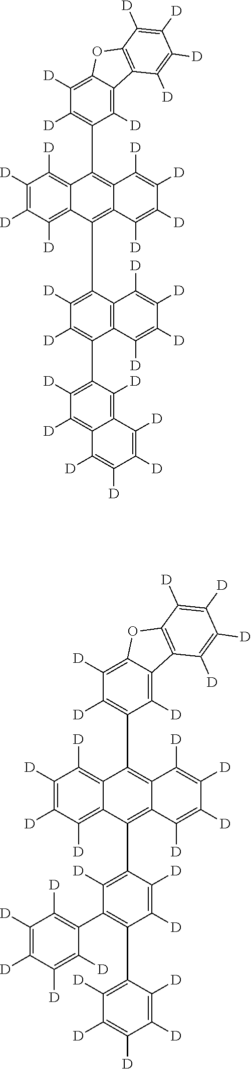 [SMILES: [2H]c1c([2H])c([2H])c2c(oc3c([2H])c([2H])c(-c4c5c([2H])c([2H])c([2H])c([2H])c5c(-c5c([2H])c([2H])c(-c6c([2H])c([2H])c7c([2H])c([2H])c([2H])c([2H])c7c6[2H])c6c([2H])c([2H])c([2H])c([2H])c56)c5c([2H])c([2H])c([2H])c([2H])c45)c([2H])c32)c1[2H] ; [2H]c1c([2H])c([2H])c(-c2c([2H])c([2H])c(-c3c4c([2H])c([2H])c([2H])c([2H])c4c(-c4c([2H])c([2H])c5oc6c([2H])c([2H])c([2H])c([2H])c6c5c4[2H])c4c([2H])c([2H])c([2H])c([2H])c34)c([2H])c2-c2c([2H])c([2H])c([2H])c([2H])c2[2H])c([2H])c1[2H]]

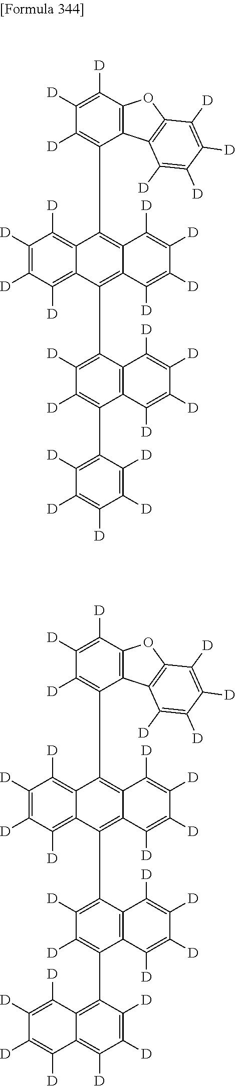 [SMILES: [2H]c1c([2H])c([2H])c(-c2c([2H])c([2H])c(-c3c4c([2H])c([2H])c([2H])c([2H])c4c(-c4c([2H])c([2H])c([2H])c5oc6c([2H])c([2H])c([2H])c([2H])c6c45)c4c([2H])c([2H])c([2H])c([2H])c34)c3c([2H])c([2H])c([2H])c([2H])c23)c([2H])c1[2H] ; [2H]c1c([2H])c([2H])c2c(oc3c([2H])c([2H])c([2H])c(-c4c5c([2H])c([2H])c([2H])c([2H])c5c(-c5c([2H])c([2H])c(-c6c([2H])c([2H])c([2H])c7c([2H])c([2H])c([2H])c([2H])c67)c6c([2H])c([2H])c([2H])c([2H])c56)c5c([2H])c([2H])c([2H])c([2H])c45)c32)c1[2H]]

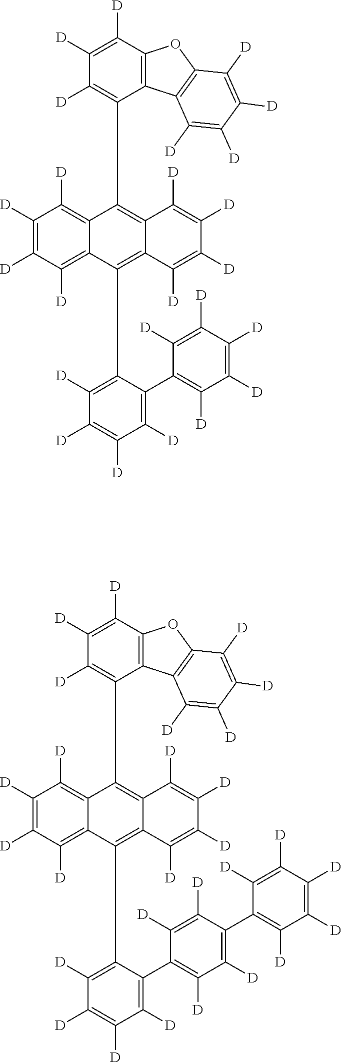 [SMILES: [2H]c1c([2H])c([2H])c(-c2c([2H])c([2H])c([2H])c([2H])c2-c2c3c([2H])c([2H])c([2H])c([2H])c3c(-c3c([2H])c([2H])c([2H])c4oc5c([2H])c([2H])c([2H])c([2H])c5c34)c3c([2H])c([2H])c([2H])c([2H])c23)c([2H])c1[2H] ; [2H]c1c([2H])c([2H])c(-c2c([2H])c([2H])c(-c3c([2H])c([2H])c([2H])c([2H])c3-c3c4c([2H])c([2H])c([2H])c([2H])c4c(-c4c([2H])c([2H])c([2H])c5oc6c([2H])c([2H])c([2H])c([2H])c6c45)c4c([2H])c([2H])c([2H])c([2H])c34)c([2H])c2[2H])c([2H])c1[2H]]

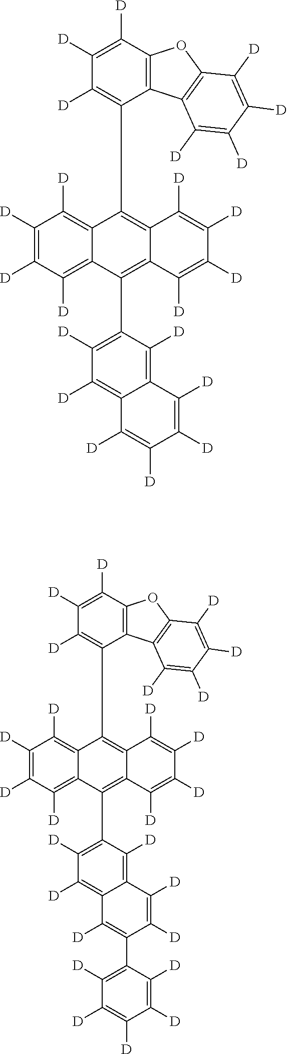 [SMILES: [2H]c1c([2H])c([2H])c2c(oc3c([2H])c([2H])c([2H])c(-c4c5c([2H])c([2H])c([2H])c([2H])c5c(-c5c([2H])c([2H])c6c([2H])c([2H])c([2H])c([2H])c6c5[2H])c5c([2H])c([2H])c([2H])c([2H])c45)c32)c1[2H] ; [2H]c1c([2H])c([2H])c(-c2c([2H])c([2H])c3c([2H])c(-c4c5c([2H])c([2H])c([2H])c([2H])c5c(-c5c([2H])c([2H])c([2H])c6oc7c([2H])c([2H])c([2H])c([2H])c7c56)c5c([2H])c([2H])c([2H])c([2H])c45)c([2H])c([2H])c3c2[2H])c([2H])c1[2H]]

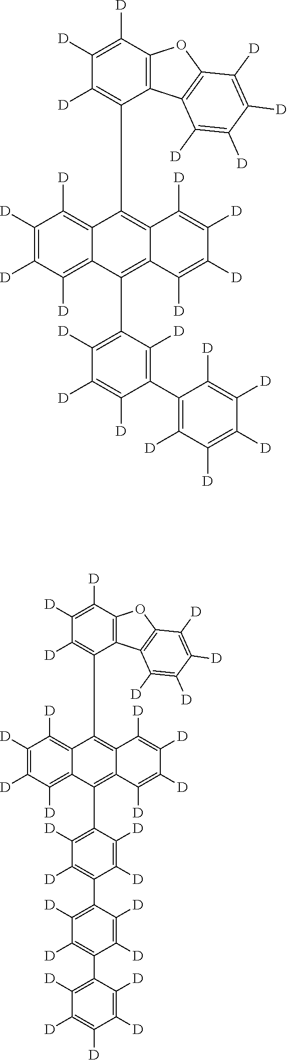 [SMILES: [2H]c1c([2H])c([2H])c(-c2c([2H])c([2H])c([2H])c(-c3c4c([2H])c([2H])c([2H])c([2H])c4c(-c4c([2H])c([2H])c([2H])c5oc6c([2H])c([2H])c([2H])c([2H])c6c45)c4c([2H])c([2H])c([2H])c([2H])c34)c2[2H])c([2H])c1[2H] ; [2H]c1c([2H])c([2H])c(-c2c([2H])c([2H])c(-c3c([2H])c([2H])c(-c4c5c([2H])c([2H])c([2H])c([2H])c5c(-c5c([2H])c([2H])c([2H])c6oc7c([2H])c([2H])c([2H])c([2H])c7c56)c5c([2H])c([2H])c([2H])c([2H])c45)c([2H])c3[2H])c([2H])c2[2H])c([2H])c1[2H]]

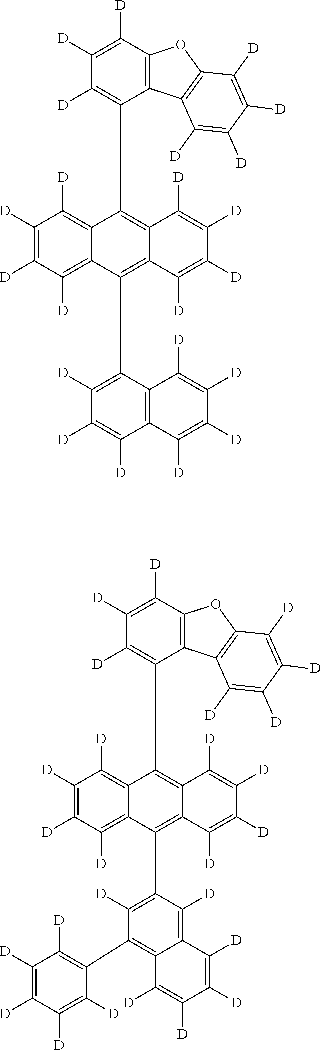 [SMILES: [2H]c1c([2H])c([2H])c2c(oc3c([2H])c([2H])c([2H])c(-c4c5c([2H])c([2H])c([2H])c([2H])c5c(-c5c([2H])c([2H])c([2H])c6c([2H])c([2H])c([2H])c([2H])c56)c5c([2H])c([2H])c([2H])c([2H])c45)c32)c1[2H] ; [2H]c1c([2H])c([2H])c(-c2c([2H])c(-c3c4c([2H])c([2H])c([2H])c([2H])c4c(-c4c([2H])c([2H])c([2H])c5oc6c([2H])c([2H])c([2H])c([2H])c6c45)c4c([2H])c([2H])c([2H])c([2H])c34)c([2H])c3c([2H])c([2H])c([2H])c([2H])c23)c([2H])c1[2H]]

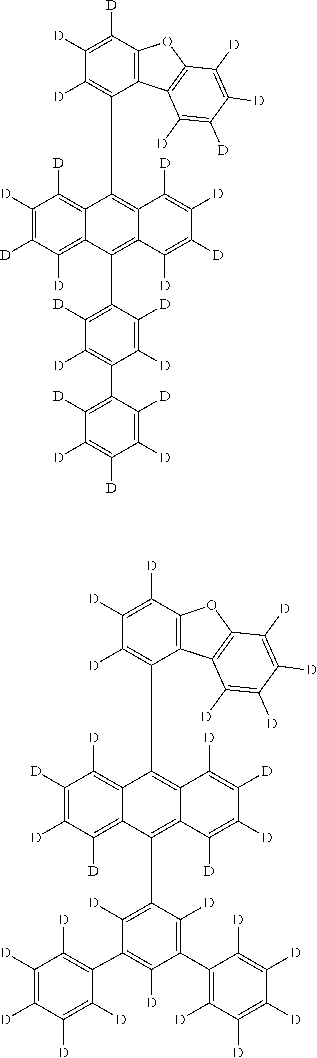 [SMILES: [2H]c1c([2H])c([2H])c(-c2c([2H])c([2H])c(-c3c4c([2H])c([2H])c([2H])c([2H])c4c(-c4c([2H])c([2H])c([2H])c5oc6c([2H])c([2H])c([2H])c([2H])c6c45)c4c([2H])c([2H])c([2H])c([2H])c34)c([2H])c2[2H])c([2H])c1[2H] ; [2H]c1c([2H])c([2H])c(-c2c([2H])c(-c3c([2H])c([2H])c([2H])c([2H])c3[2H])c([2H])c(-c3c4c([2H])c([2H])c([2H])c([2H])c4c(-c4c([2H])c([2H])c([2H])c5oc6c([2H])c([2H])c([2H])c([2H])c6c45)c4c([2H])c([2H])c([2H])c([2H])c34)c2[2H])c([2H])c1[2H]]

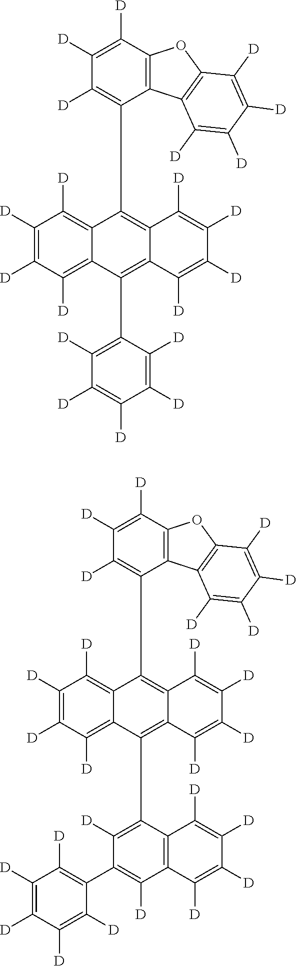 [SMILES: [2H]c1c([2H])c([2H])c(-c2c3c([2H])c([2H])c([2H])c([2H])c3c(-c3c([2H])c([2H])c([2H])c4oc5c([2H])c([2H])c([2H])c([2H])c5c34)c3c([2H])c([2H])c([2H])c([2H])c23)c([2H])c1[2H] ; [2H]c1c([2H])c([2H])c(-c2c([2H])c(-c3c4c([2H])c([2H])c([2H])c([2H])c4c(-c4c([2H])c([2H])c([2H])c5oc6c([2H])c([2H])c([2H])c([2H])c6c45)c4c([2H])c([2H])c([2H])c([2H])c34)c3c([2H])c([2H])c([2H])c([2H])c3c2[2H])c([2H])c1[2H]]

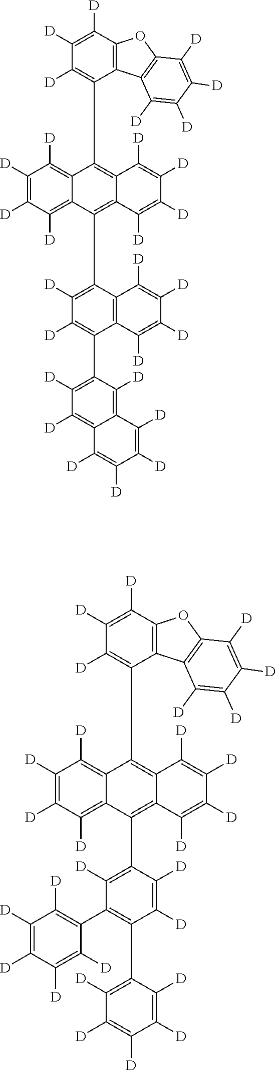 [SMILES: [2H]c1c([2H])c([2H])c2c(oc3c([2H])c([2H])c([2H])c(-c4c5c([2H])c([2H])c([2H])c([2H])c5c(-c5c([2H])c([2H])c(-c6c([2H])c([2H])c7c([2H])c([2H])c([2H])c([2H])c7c6[2H])c6c([2H])c([2H])c([2H])c([2H])c56)c5c([2H])c([2H])c([2H])c([2H])c45)c32)c1[2H] ; [2H]c1c([2H])c([2H])c(-c2c([2H])c([2H])c(-c3c4c([2H])c([2H])c([2H])c([2H])c4c(-c4c([2H])c([2H])c([2H])c5oc6c([2H])c([2H])c([2H])c([2H])c6c45)c4c([2H])c([2H])c([2H])c([2H])c34)c([2H])c2-c2c([2H])c([2H])c([2H])c([2H])c2[2H])c([2H])c1[2H]]

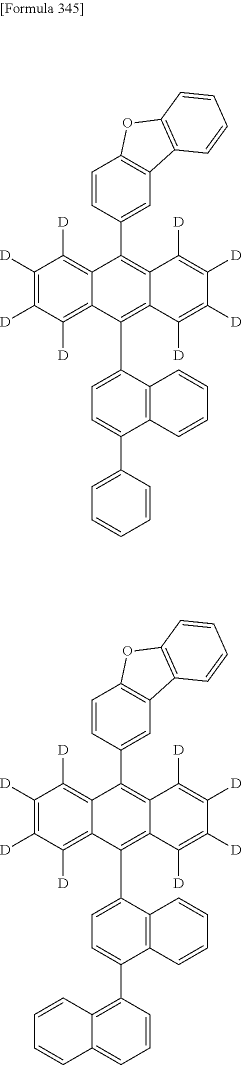 [SMILES: [2H]c1c([2H])c([2H])c2c(-c3ccc(-c4ccccc4)c4ccccc34)c3c([2H])c([2H])c([2H])c([2H])c3c(-c3ccc4oc5ccccc5c4c3)c2c1[2H] ; [2H]c1c([2H])c([2H])c2c(-c3ccc(-c4cccc5ccccc45)c4ccccc34)c3c([2H])c([2H])c([2H])c([2H])c3c(-c3ccc4oc5ccccc5c4c3)c2c1[2H]]

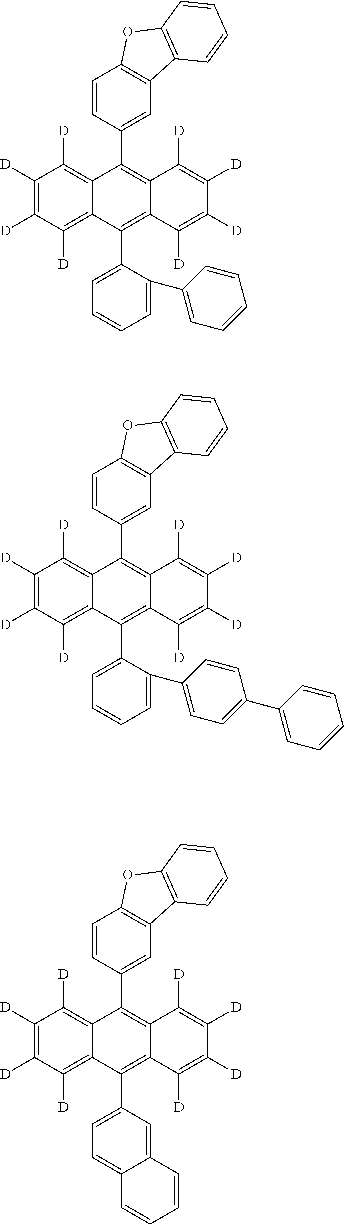 [SMILES: [2H]c1c([2H])c([2H])c2c(-c3ccccc3-c3ccccc3)c3c([2H])c([2H])c([2H])c([2H])c3c(-c3ccc4oc5ccccc5c4c3)c2c1[2H] ; [2H]c1c([2H])c([2H])c2c(-c3ccccc3-c3ccc(-c4ccccc4)cc3)c3c([2H])c([2H])c([2H])c([2H])c3c(-c3ccc4oc5ccccc5c4c3)c2c1[2H] ; [2H]c1c([2H])c([2H])c2c(-c3ccc4oc5ccccc5c4c3)c3c([2H])c([2H])c([2H])c([2H])c3c(-c3ccc4ccccc4c3)c2c1[2H]]

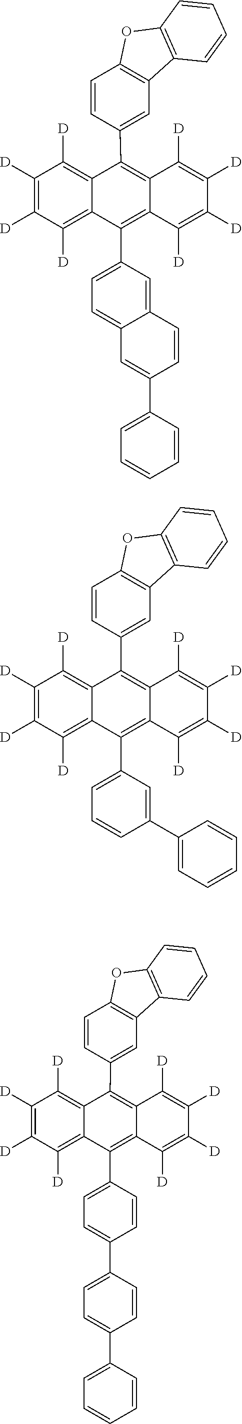

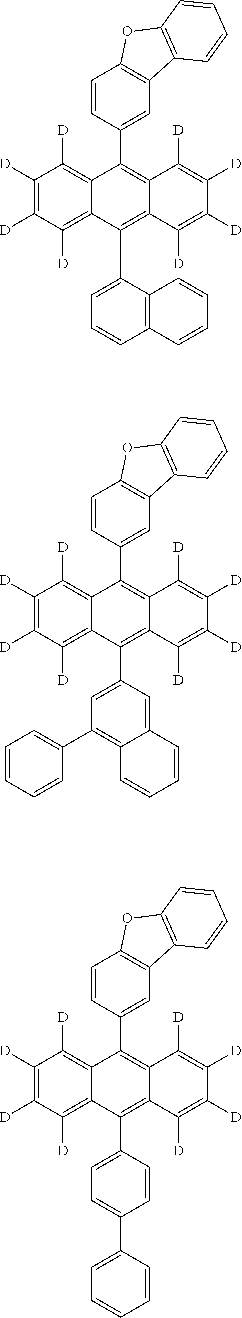 [SMILES: [2H]c1c([2H])c([2H])c2c(-c3cccc4ccccc34)c3c([2H])c([2H])c([2H])c([2H])c3c(-c3ccc4oc5ccccc5c4c3)c2c1[2H] ; [2H]c1c([2H])c([2H])c2c(-c3ccc4oc5ccccc5c4c3)c3c([2H])c([2H])c([2H])c([2H])c3c(-c3cc(-c4ccccc4)c4ccccc4c3)c2c1[2H] ; [2H]c1c([2H])c([2H])c2c(-c3ccc4oc5ccccc5c4c3)c3c([2H])c([2H])c([2H])c([2H])c3c(-c3ccc(-c4ccccc4)cc3)c2c1[2H]]

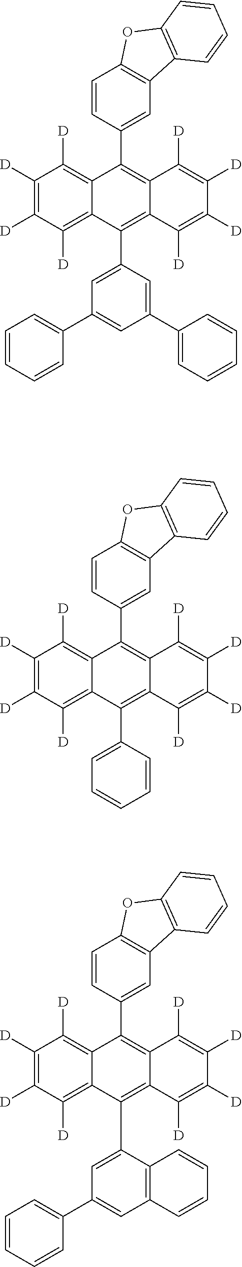 [SMILES: [2H]c1c([2H])c([2H])c2c(-c3ccc4oc5ccccc5c4c3)c3c([2H])c([2H])c([2H])c([2H])c3c(-c3cc(-c4ccccc4)cc(-c4ccccc4)c3)c2c1[2H] ; [2H]c1c([2H])c([2H])c2c(-c3ccc4oc5ccccc5c4c3)c3c([2H])c([2H])c([2H])c([2H])c3c(-c3ccccc3)c2c1[2H] ; [2H]c1c([2H])c([2H])c2c(-c3cc(-c4ccccc4)cc4ccccc34)c3c([2H])c([2H])c([2H])c([2H])c3c(-c3ccc4oc5ccccc5c4c3)c2c1[2H]]

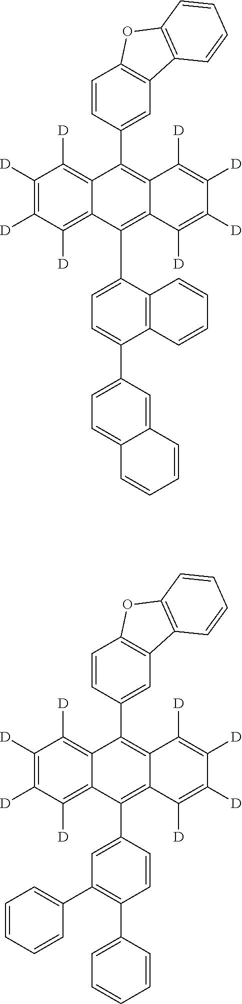 [SMILES: [2H]c1c([2H])c([2H])c2c(-c3ccc(-c4ccc5ccccc5c4)c4ccccc34)c3c([2H])c([2H])c([2H])c([2H])c3c(-c3ccc4oc5ccccc5c4c3)c2c1[2H] ; [2H]c1c([2H])c([2H])c2c(-c3ccc4oc5ccccc5c4c3)c3c([2H])c([2H])c([2H])c([2H])c3c(-c3ccc(-c4ccccc4)c(-c4ccccc4)c3)c2c1[2H]]

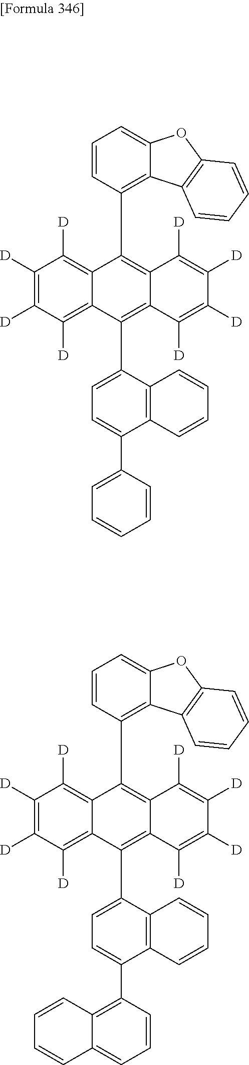 [SMILES: [2H]c1c([2H])c([2H])c2c(-c3cccc4oc5ccccc5c34)c3c([2H])c([2H])c([2H])c([2H])c3c(-c3ccc(-c4ccccc4)c4ccccc34)c2c1[2H] ; [2H]c1c([2H])c([2H])c2c(-c3cccc4oc5ccccc5c34)c3c([2H])c([2H])c([2H])c([2H])c3c(-c3ccc(-c4cccc5ccccc45)c4ccccc34)c2c1[2H]]

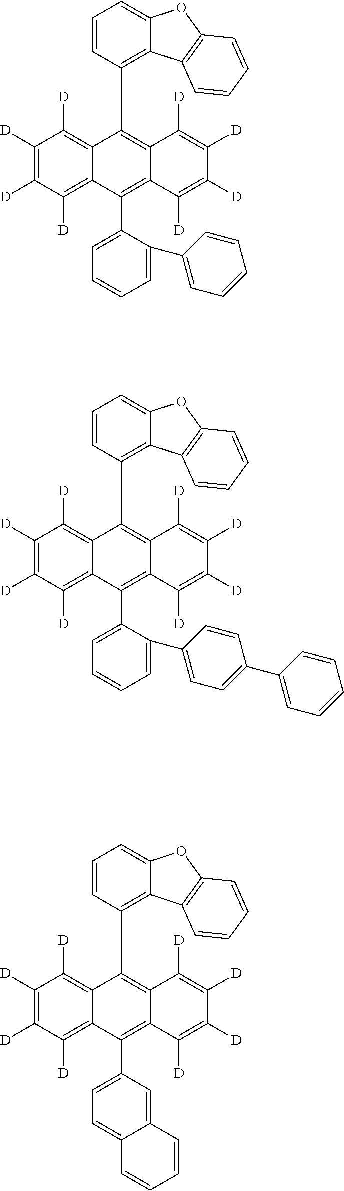 [SMILES: [2H]c1c([2H])c([2H])c2c(-c3cccc4oc5ccccc5c34)c3c([2H])c([2H])c([2H])c([2H])c3c(-c3ccccc3-c3ccccc3)c2c1[2H] ; [2H]c1c([2H])c([2H])c2c(-c3cccc4oc5ccccc5c34)c3c([2H])c([2H])c([2H])c([2H])c3c(-c3ccccc3-c3ccc(-c4ccccc4)cc3)c2c1[2H] ; [2H]c1c([2H])c([2H])c2c(-c3cccc4oc5ccccc5c34)c3c([2H])c([2H])c([2H])c([2H])c3c(-c3ccc4ccccc4c3)c2c1[2H]]

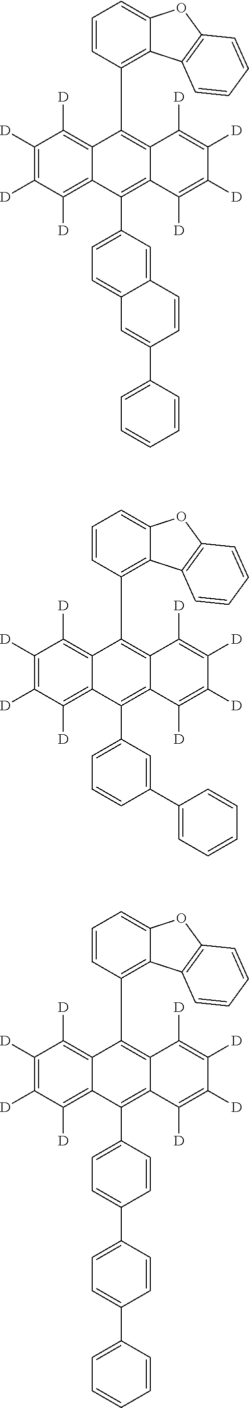 [SMILES: [2H]c1c([2H])c([2H])c2c(-c3cccc4oc5ccccc5c34)c3c([2H])c([2H])c([2H])c([2H])c3c(-c3ccc4cc(-c5ccccc5)ccc4c3)c2c1[2H] ; [2H]c1c([2H])c([2H])c2c(-c3cccc4oc5ccccc5c34)c3c([2H])c([2H])c([2H])c([2H])c3c(-c3cccc(-c4ccccc4)c3)c2c1[2H] ; [2H]c1c([2H])c([2H])c2c(-c3cccc4oc5ccccc5c34)c3c([2H])c([2H])c([2H])c([2H])c3c(-c3ccc(-c4ccc(-c5ccccc5)cc4)cc3)c2c1[2H]]

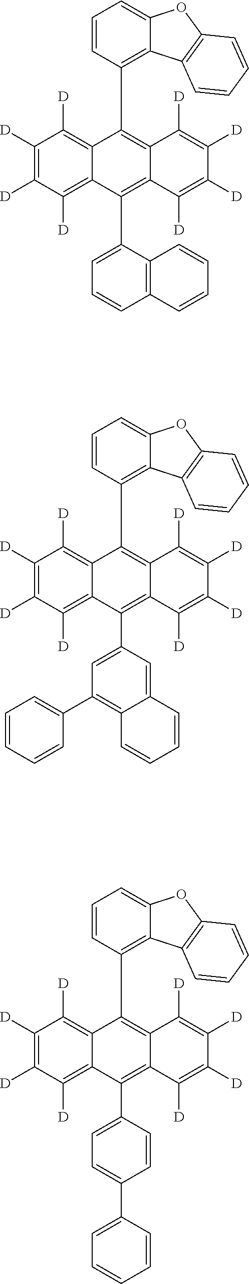 [SMILES: [2H]c1c([2H])c([2H])c2c(-c3cccc4oc5ccccc5c34)c3c([2H])c([2H])c([2H])c([2H])c3c(-c3cccc4ccccc34)c2c1[2H] ; [2H]c1c([2H])c([2H])c2c(-c3cccc4oc5ccccc5c34)c3c([2H])c([2H])c([2H])c([2H])c3c(-c3cc(-c4ccccc4)c4ccccc4c3)c2c1[2H] ; [2H]c1c([2H])c([2H])c2c(-c3cccc4oc5ccccc5c34)c3c([2H])c([2H])c([2H])c([2H])c3c(-c3ccc(-c4ccccc4)cc3)c2c1[2H]]

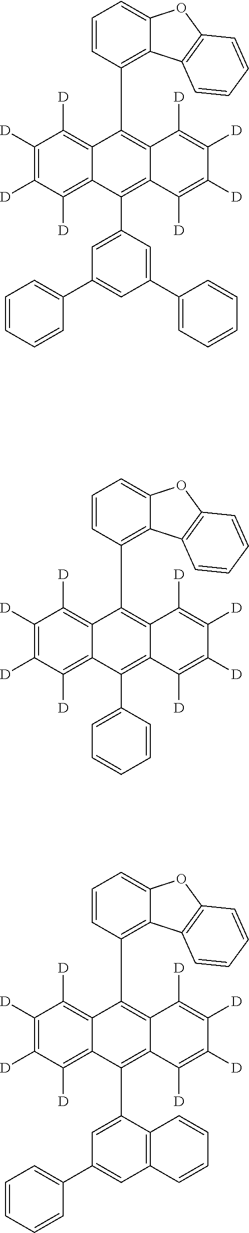 [SMILES: [2H]c1c([2H])c([2H])c2c(-c3cccc4oc5ccccc5c34)c3c([2H])c([2H])c([2H])c([2H])c3c(-c3cc(-c4ccccc4)cc(-c4ccccc4)c3)c2c1[2H] ; [2H]c1c([2H])c([2H])c2c(-c3cccc4oc5ccccc5c34)c3c([2H])c([2H])c([2H])c([2H])c3c(-c3ccccc3)c2c1[2H] ; [2H]c1c([2H])c([2H])c2c(-c3cccc4oc5ccccc5c34)c3c([2H])c([2H])c([2H])c([2H])c3c(-c3cc(-c4ccccc4)cc4ccccc34)c2c1[2H]]

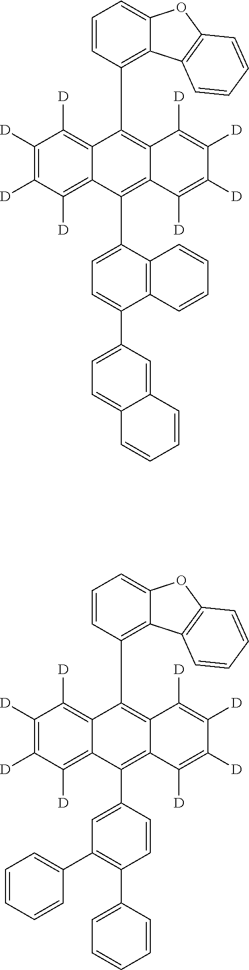 [SMILES: [2H]c1c([2H])c([2H])c2c(-c3cccc4oc5ccccc5c34)c3c([2H])c([2H])c([2H])c([2H])c3c(-c3ccc(-c4ccc5ccccc5c4)c4ccccc34)c2c1[2H] ; [2H]c1c([2H])c([2H])c2c(-c3cccc4oc5ccccc5c34)c3c([2H])c([2H])c([2H])c([2H])c3c(-c3ccc(-c4ccccc4)c(-c4ccccc4)c3)c2c1[2H]]

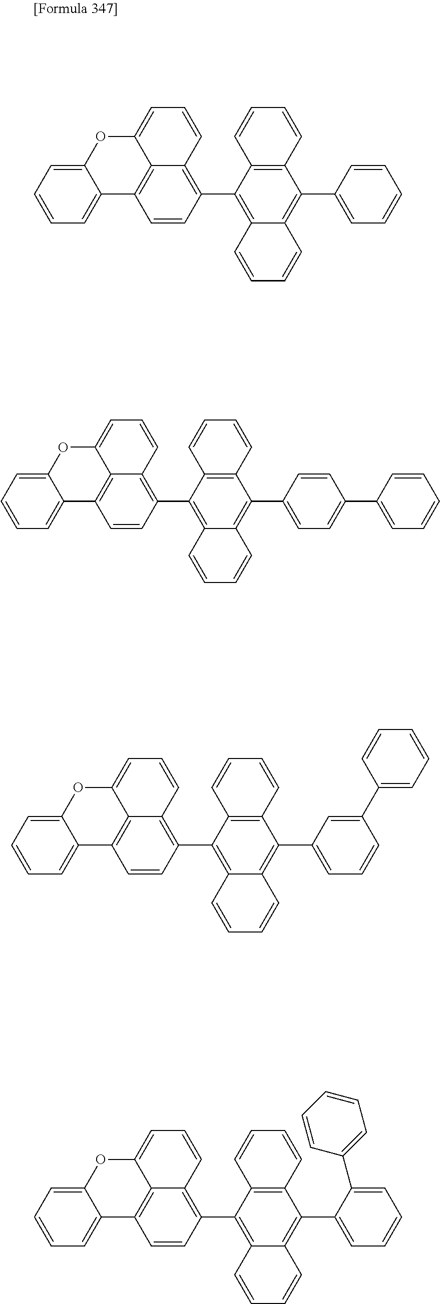 [SMILES: c1ccc(-c2c3ccccc3c(-c3ccc4c5c(cccc35)Oc3ccccc3-4)c3ccccc23)cc1 ; c1ccc(-c2ccc(-c3c4ccccc4c(-c4ccc5c6c(cccc46)Oc4ccccc4-5)c4ccccc34)cc2)cc1 ; c1ccc(-c2cccc(-c3c4ccccc4c(-c4ccc5c6c(cccc46)Oc4ccccc4-5)c4ccccc34)c2)cc1 ; c1ccc(-c2ccccc2-c2c3ccccc3c(-c3ccc4c5c(cccc35)Oc3ccccc3-4)c3ccccc23)cc1]

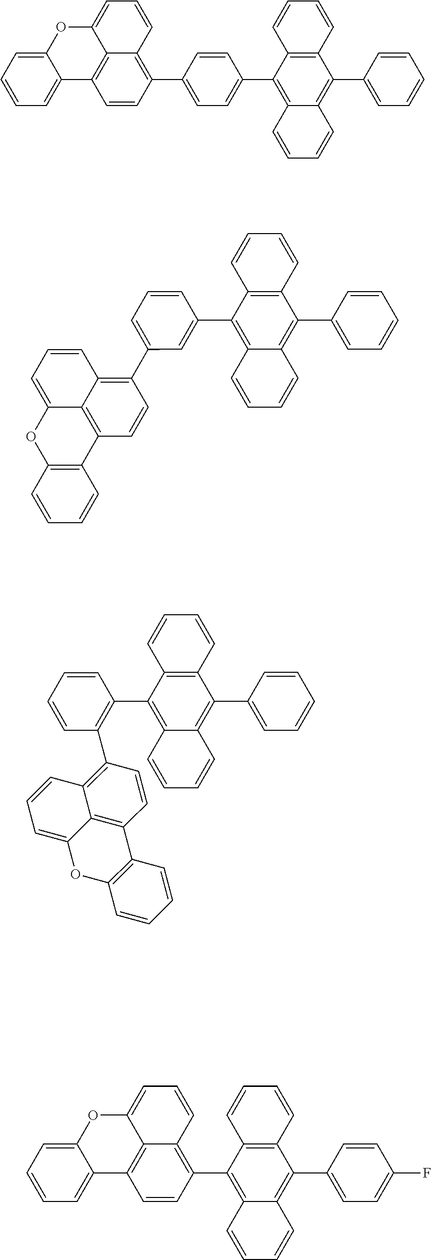 [SMILES: c1ccc(-c2c3ccccc3c(-c3ccc(-c4ccc5c6c(cccc46)Oc4ccccc4-5)cc3)c3ccccc23)cc1 ; c1ccc(-c2c3ccccc3c(-c3cccc(-c4ccc5c6c(cccc46)Oc4ccccc4-5)c3)c3ccccc23)cc1 ; c1ccc(-c2c3ccccc3c(-c3ccccc3-c3ccc4c5c(cccc35)Oc3ccccc3-4)c3ccccc23)cc1 ; Fc1ccc(-c2c3ccccc3c(-c3ccc4c5c(cccc35)Oc3ccccc3-4)c3ccccc23)cc1]

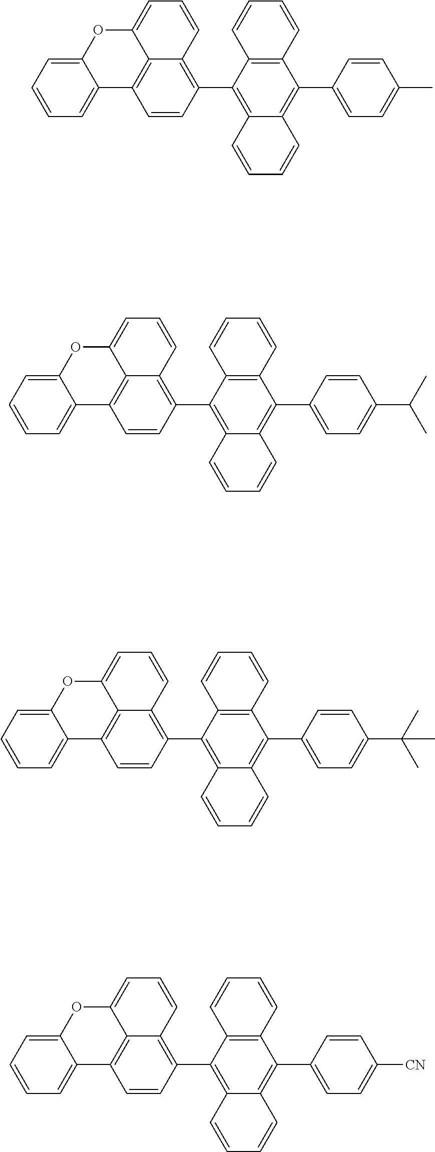 [SMILES: Cc1ccc(-c2c3ccccc3c(-c3ccc4c5c(cccc35)Oc3ccccc3-4)c3ccccc23)cc1 ; CC(C)c1ccc(-c2c3ccccc3c(-c3ccc4c5c(cccc35)Oc3ccccc3-4)c3ccccc23)cc1 ; CC(C)(C)c1ccc(-c2c3ccccc3c(-c3ccc4c5c(cccc35)Oc3ccccc3-4)c3ccccc23)cc1 ; N#Cc1ccc(-c2c3ccccc3c(-c3ccc4c5c(cccc35)Oc3ccccc3-4)c3ccccc23)cc1]

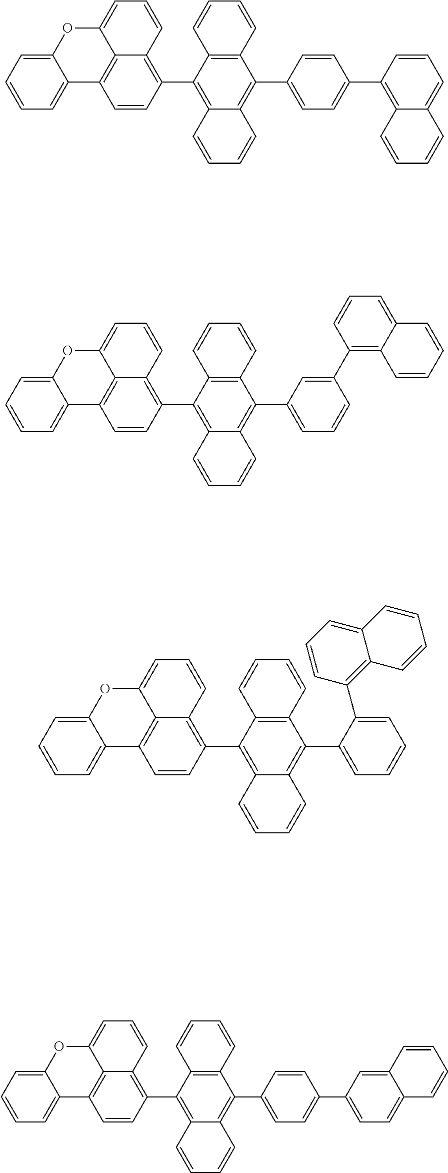 [SMILES: c1ccc2c(c1)Oc1cccc3c(-c4c5ccccc5c(-c5ccc(-c6cccc7ccccc67)cc5)c5ccccc45)ccc-2c13 ; c1cc(-c2cccc3ccccc23)cc(-c2c3ccccc3c(-c3ccc4c5c(cccc35)Oc3ccccc3-4)c3ccccc23)c1 ; c1ccc2c(c1)Oc1cccc3c(-c4c5ccccc5c(-c5ccccc5-c5cccc6ccccc56)c5ccccc45)ccc-2c13 ; c1ccc2c(c1)Oc1cccc3c(-c4c5ccccc5c(-c5ccc(-c6ccc7ccccc7c6)cc5)c5ccccc45)ccc-2c13]

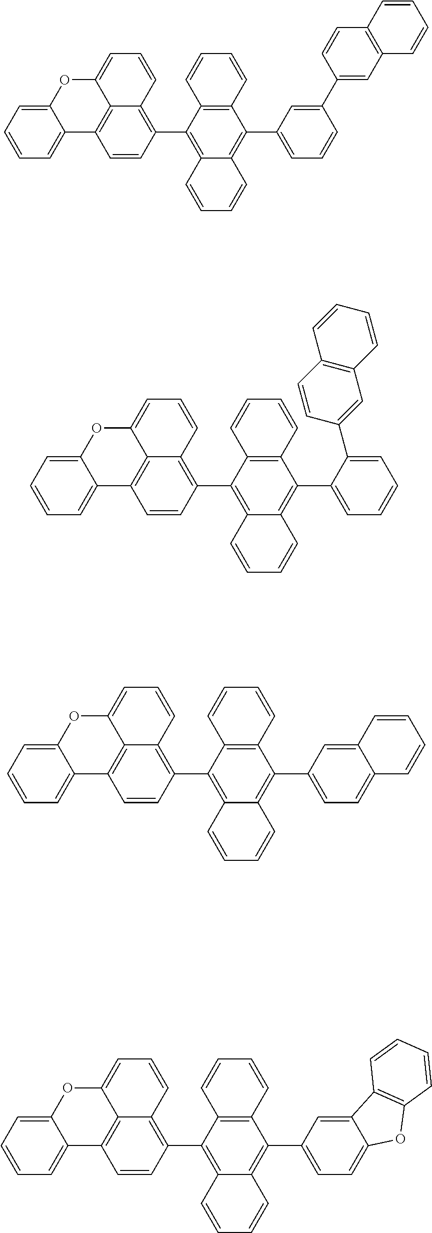 [SMILES: c1cc(-c2ccc3ccccc3c2)cc(-c2c3ccccc3c(-c3ccc4c5c(cccc35)Oc3ccccc3-4)c3ccccc23)c1 ; c1ccc2c(c1)Oc1cccc3c(-c4c5ccccc5c(-c5ccccc5-c5ccc6ccccc6c5)c5ccccc45)ccc-2c13 ; c1ccc2c(c1)Oc1cccc3c(-c4c5ccccc5c(-c5ccc6ccccc6c5)c5ccccc45)ccc-2c13 ; c1ccc2c(c1)Oc1cccc3c(-c4c5ccccc5c(-c5ccc6oc7ccccc7c6c5)c5ccccc45)ccc-2c13]

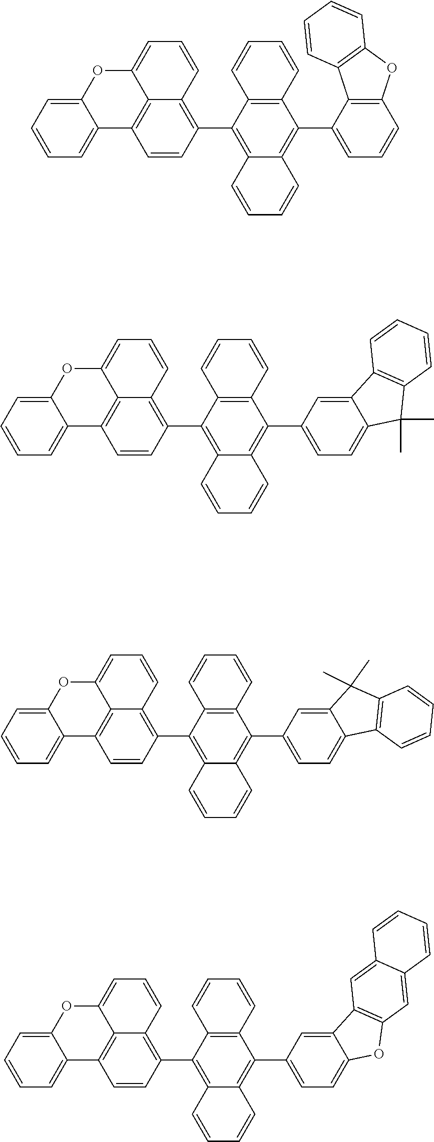 [SMILES: c1ccc2c(c1)Oc1cccc3c(-c4c5ccccc5c(-c5cccc6oc7ccccc7c56)c5ccccc45)ccc-2c13 ; CC1(C)c2ccccc2-c2cc(-c3c4ccccc4c(-c4ccc5c6c(cccc46)Oc4ccccc4-5)c4ccccc34)ccc21 ; CC1(C)c2ccccc2-c2ccc(-c3c4ccccc4c(-c4ccc5c6c(cccc46)Oc4ccccc4-5)c4ccccc34)cc21 ; c1ccc2c(c1)Oc1cccc3c(-c4c5ccccc5c(-c5ccc6oc7cc8ccccc8cc7c6c5)c5ccccc45)ccc-2c13]

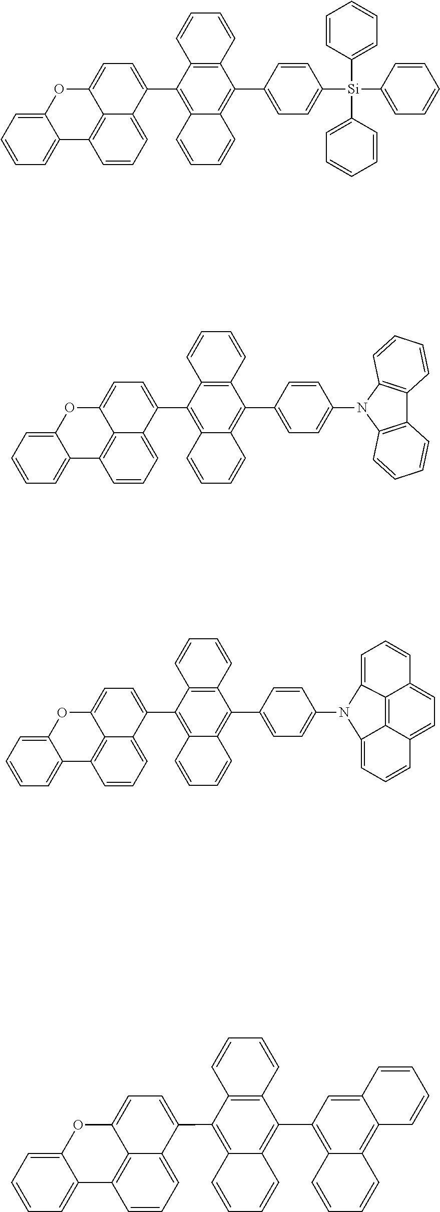 [SMILES: c1ccc([Si](c2ccccc2)(c2ccccc2)c2ccc(-c3c4ccccc4c(-c4ccc5c6c(cccc46)-c4ccccc4O5)c4ccccc34)cc2)cc1 ; c1ccc2c(c1)Oc1ccc(-c3c4ccccc4c(-c4ccc(-n5c6ccccc6c6ccccc65)cc4)c4ccccc34)c3cccc-2c13 ; c1ccc2c(c1)Oc1ccc(-c3c4ccccc4c(-c4ccc(-n5c6cccc7ccc8cccc5c8c76)cc4)c4ccccc34)c3cccc-2c13 ; c1ccc2c(c1)Oc1ccc(-c3c4ccccc4c(-c4cc5ccccc5c5ccccc45)c4ccccc34)c3cccc-2c13]

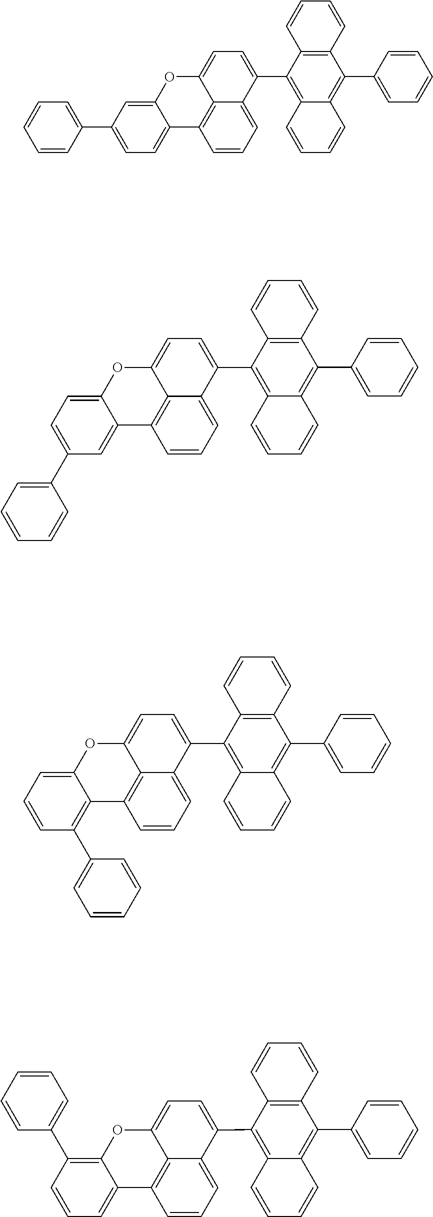 [SMILES: c1ccc(-c2ccc3c(c2)Oc2ccc(-c4c5ccccc5c(-c5ccccc5)c5ccccc45)c4cccc-3c24)cc1 ; c1ccc(-c2ccc3c(c2)-c2cccc4c(-c5c6ccccc6c(-c6ccccc6)c6ccccc56)ccc(c24)O3)cc1 ; c1ccc(-c2cccc3c2-c2cccc4c(-c5c6ccccc6c(-c6ccccc6)c6ccccc56)ccc(c24)O3)cc1 ; c1ccc(-c2cccc3c2Oc2ccc(-c4c5ccccc5c(-c5ccccc5)c5ccccc45)c4cccc-3c24)cc1]

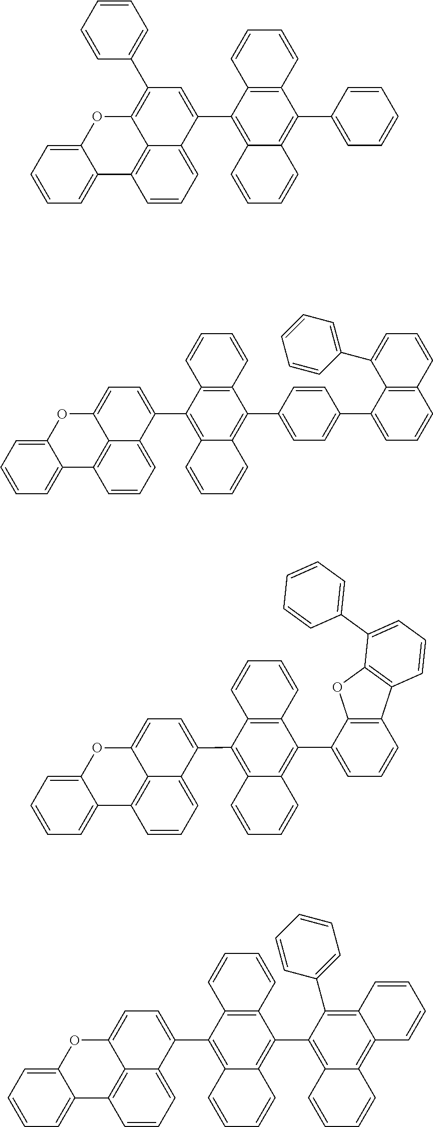 [SMILES: c1ccc(-c2cc(-c3c4ccccc4c(-c4ccccc4)c4ccccc34)c3cccc4c3c2Oc2ccccc2-4)cc1 ; c1ccc(-c2cccc3cccc(-c4ccc(-c5c6ccccc6c(-c6ccc7c8c(cccc68)-c6ccccc6O7)c6ccccc56)cc4)c23)cc1 ; c1ccc(-c2cccc3c2oc2c(-c4c5ccccc5c(-c5ccc6c7c(cccc57)-c5ccccc5O6)c5ccccc45)cccc23)cc1 ; c1ccc(-c2c(-c3c4ccccc4c(-c4ccc5c6c(cccc46)-c4ccccc4O5)c4ccccc34)c3ccccc3c3ccccc23)cc1]

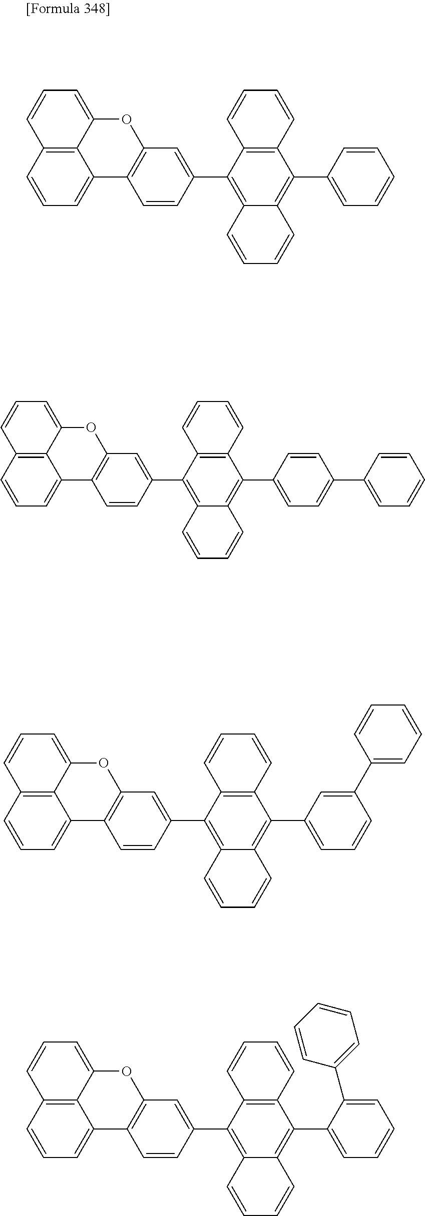 [SMILES: c1ccc(-c2c3ccccc3c(-c3ccc4c(c3)Oc3cccc5cccc-4c35)c3ccccc23)cc1 ; c1ccc(-c2ccc(-c3c4ccccc4c(-c4ccc5c(c4)Oc4cccc6cccc-5c46)c4ccccc34)cc2)cc1 ; c1ccc(-c2cccc(-c3c4ccccc4c(-c4ccc5c(c4)Oc4cccc6cccc-5c46)c4ccccc34)c2)cc1 ; c1ccc(-c2ccccc2-c2c3ccccc3c(-c3ccc4c(c3)Oc3cccc5cccc-4c35)c3ccccc23)cc1]

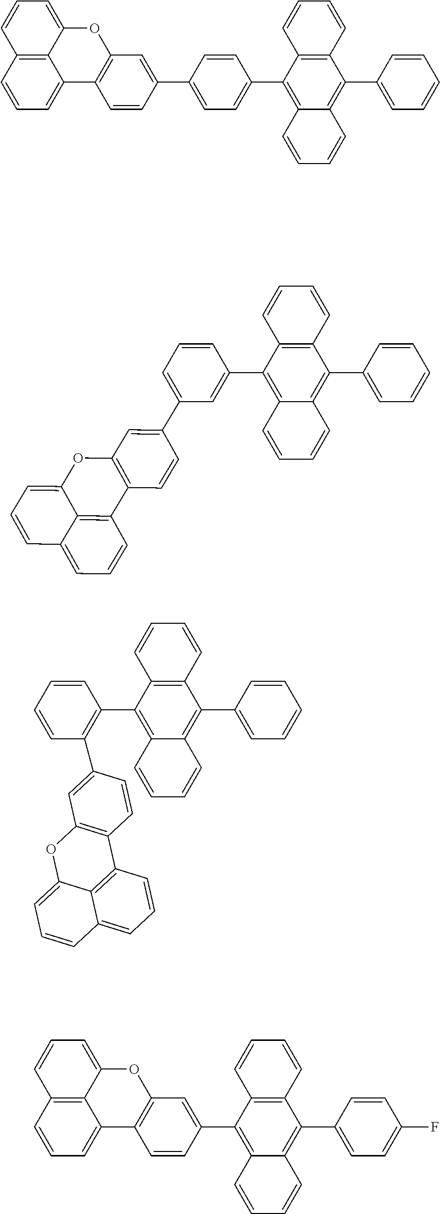 [SMILES: c1ccc(-c2c3ccccc3c(-c3ccc(-c4ccc5c(c4)Oc4cccc6cccc-5c46)cc3)c3ccccc23)cc1 ; c1ccc(-c2c3ccccc3c(-c3cccc(-c4ccc5c(c4)Oc4cccc6cccc-5c46)c3)c3ccccc23)cc1 ; c1ccc(-c2c3ccccc3c(-c3ccccc3-c3ccc4c(c3)Oc3cccc5cccc-4c35)c3ccccc23)cc1 ; Fc1ccc(-c2c3ccccc3c(-c3ccc4c(c3)Oc3cccc5cccc-4c35)c3ccccc23)cc1]

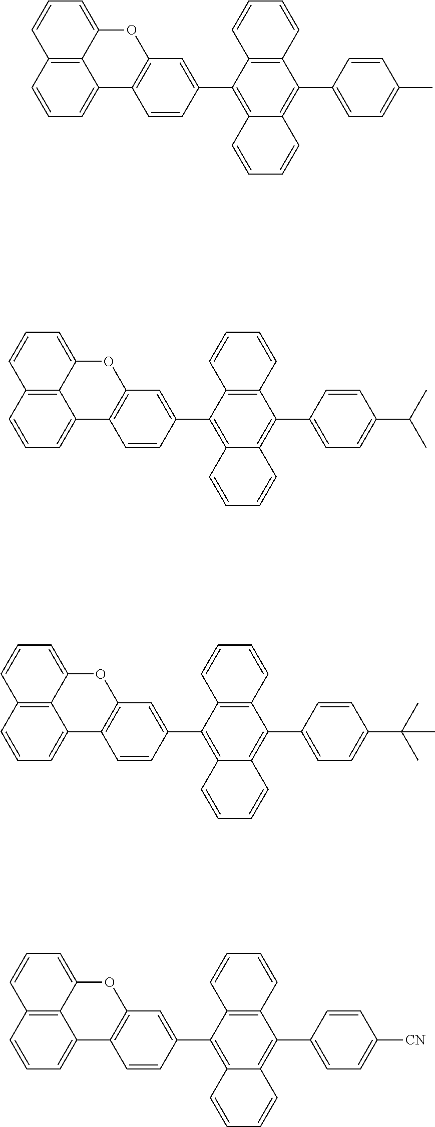 [SMILES: Cc1ccc(-c2c3ccccc3c(-c3ccc4c(c3)Oc3cccc5cccc-4c35)c3ccccc23)cc1 ; CC(C)c1ccc(-c2c3ccccc3c(-c3ccc4c(c3)Oc3cccc5cccc-4c35)c3ccccc23)cc1 ; CC(C)(C)c1ccc(-c2c3ccccc3c(-c3ccc4c(c3)Oc3cccc5cccc-4c35)c3ccccc23)cc1 ; N#Cc1ccc(-c2c3ccccc3c(-c3ccc4c(c3)Oc3cccc5cccc-4c35)c3ccccc23)cc1]

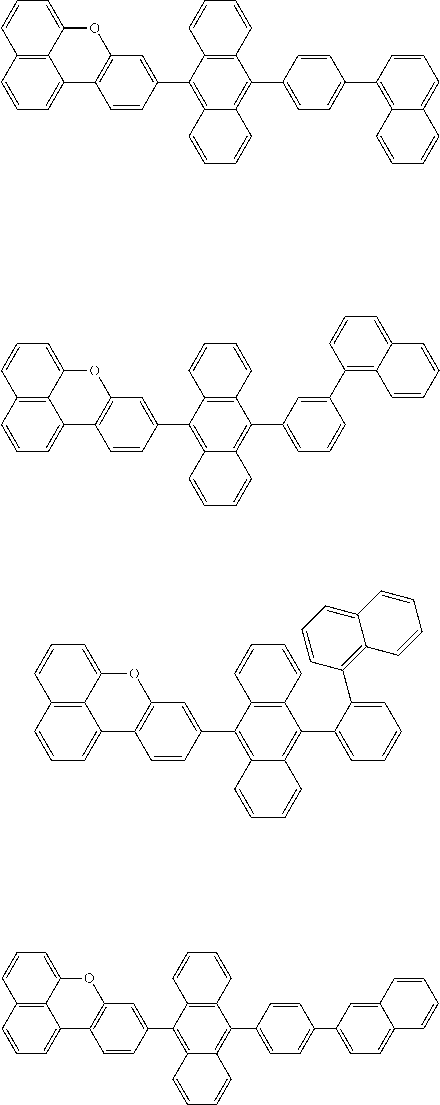 [SMILES: c1ccc2c(-c3ccc(-c4c5ccccc5c(-c5ccc6c(c5)Oc5cccc7cccc-6c57)c5ccccc45)cc3)cccc2c1 ; c1cc(-c2cccc3ccccc23)cc(-c2c3ccccc3c(-c3ccc4c(c3)Oc3cccc5cccc-4c35)c3ccccc23)c1 ; c1ccc(-c2c3ccccc3c(-c3ccc4c(c3)Oc3cccc5cccc-4c35)c3ccccc23)c(-c2cccc3ccccc23)c1 ; c1ccc2cc(-c3ccc(-c4c5ccccc5c(-c5ccc6c(c5)Oc5cccc7cccc-6c57)c5ccccc45)cc3)ccc2c1]

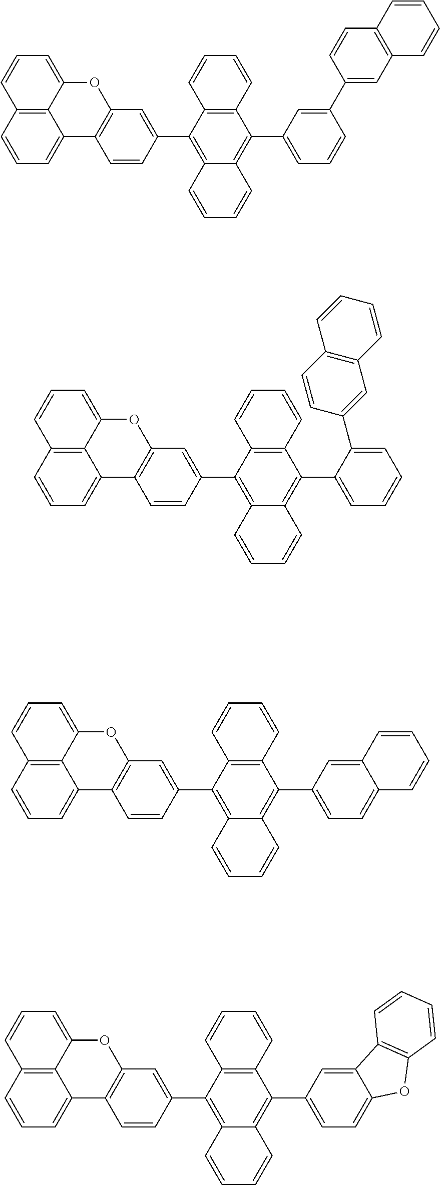 [SMILES: c1cc(-c2ccc3ccccc3c2)cc(-c2c3ccccc3c(-c3ccc4c(c3)Oc3cccc5cccc-4c35)c3ccccc23)c1 ; c1ccc(-c2c3ccccc3c(-c3ccc4c(c3)Oc3cccc5cccc-4c35)c3ccccc23)c(-c2ccc3ccccc3c2)c1 ; c1ccc2cc(-c3c4ccccc4c(-c4ccc5c(c4)Oc4cccc6cccc-5c46)c4ccccc34)ccc2c1 ; c1cc2c3c(cccc3c1)-c1ccc(-c3c4ccccc4c(-c4ccc5oc6ccccc6c5c4)c4ccccc34)cc1O2]

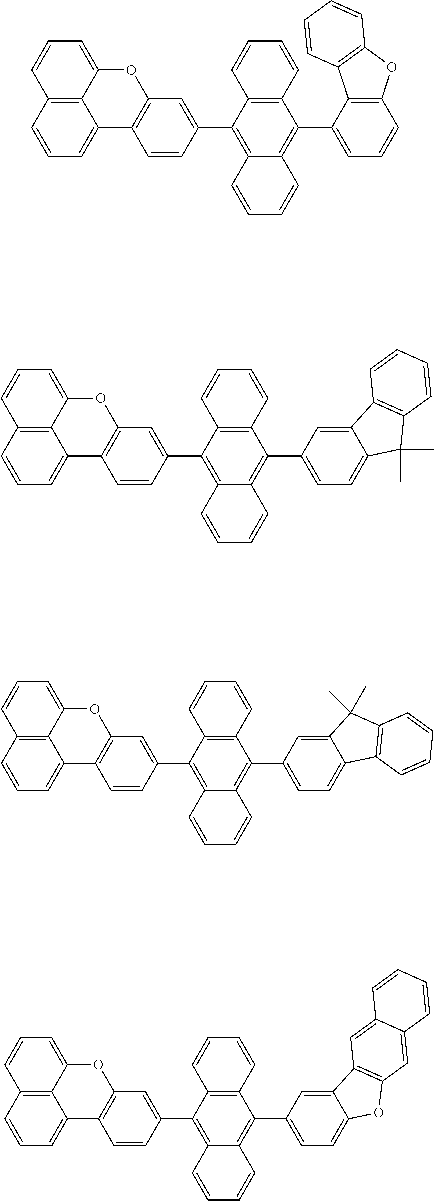 [SMILES: c1cc2c3c(cccc3c1)-c1ccc(-c3c4ccccc4c(-c4cccc5oc6ccccc6c45)c4ccccc34)cc1O2 ; CC1(C)c2ccccc2-c2cc(-c3c4ccccc4c(-c4ccc5c(c4)Oc4cccc6cccc-5c46)c4ccccc34)ccc21 ; CC1(C)c2ccccc2-c2ccc(-c3c4ccccc4c(-c4ccc5c(c4)Oc4cccc6cccc-5c46)c4ccccc34)cc21 ; c1ccc2cc3c(cc2c1)oc1ccc(-c2c4ccccc4c(-c4ccc5c(c4)Oc4cccc6cccc-5c46)c4ccccc24)cc13]

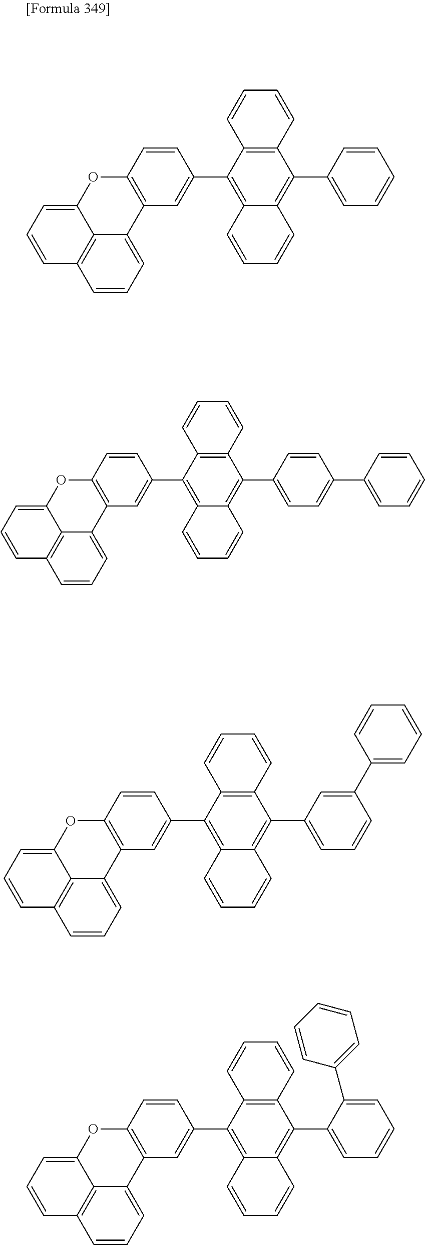 [SMILES: c1ccc(-c2c3ccccc3c(-c3ccc4c(c3)-c3cccc5cccc(c35)O4)c3ccccc23)cc1 ; c1ccc(-c2ccc(-c3c4ccccc4c(-c4ccc5c(c4)-c4cccc6cccc(c46)O5)c4ccccc34)cc2)cc1 ; c1ccc(-c2cccc(-c3c4ccccc4c(-c4ccc5c(c4)-c4cccc6cccc(c46)O5)c4ccccc34)c2)cc1 ; c1ccc(-c2ccccc2-c2c3ccccc3c(-c3ccc4c(c3)-c3cccc5cccc(c35)O4)c3ccccc23)cc1]

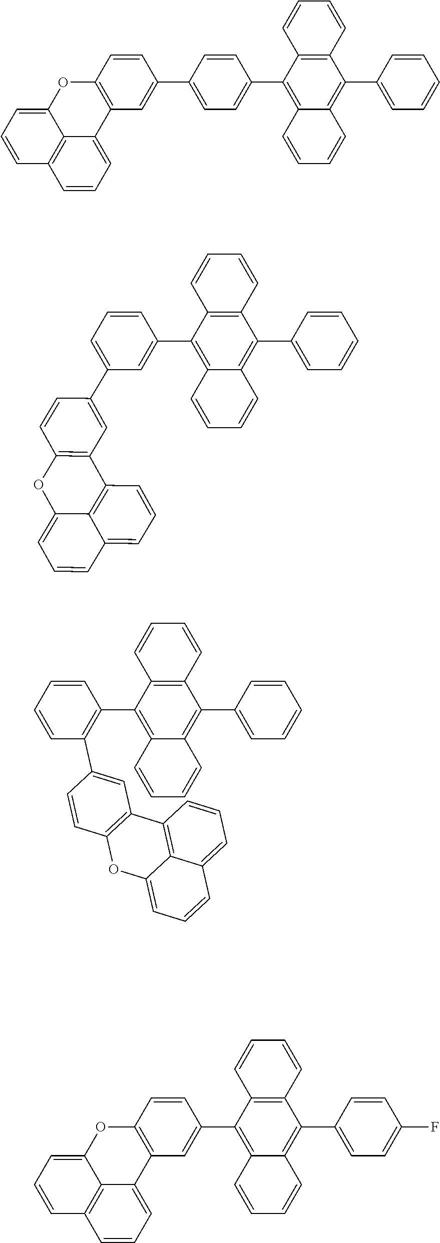 [SMILES: c1ccc(-c2c3ccccc3c(-c3ccc(-c4ccc5c(c4)-c4cccc6cccc(c46)O5)cc3)c3ccccc23)cc1 ; c1ccc(-c2c3ccccc3c(-c3cccc(-c4ccc5c(c4)-c4cccc6cccc(c46)O5)c3)c3ccccc23)cc1 ; c1ccc(-c2c3ccccc3c(-c3ccccc3-c3ccc4c(c3)-c3cccc5cccc(c35)O4)c3ccccc23)cc1 ; Fc1ccc(-c2c3ccccc3c(-c3ccc4c(c3)-c3cccc5cccc(c35)O4)c3ccccc23)cc1]

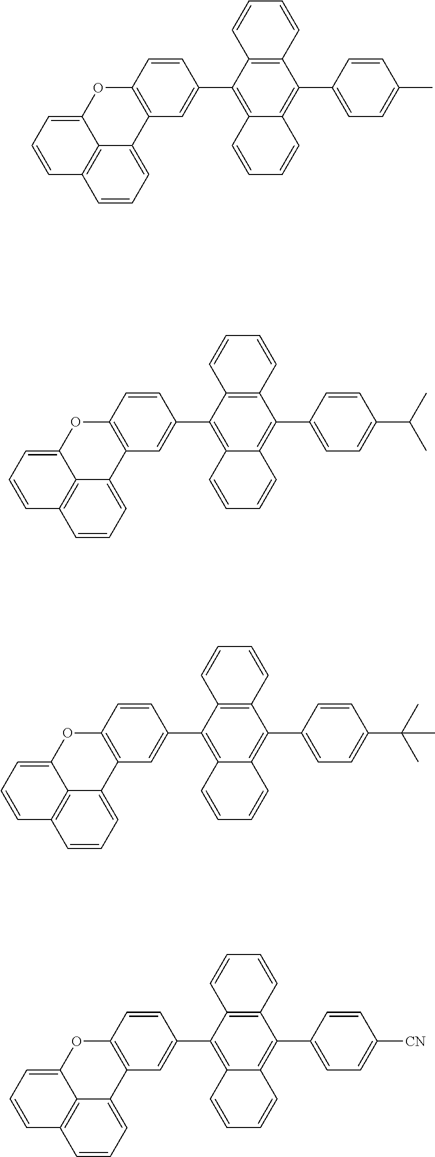 [SMILES: Cc1ccc(-c2c3ccccc3c(-c3ccc4c(c3)-c3cccc5cccc(c35)O4)c3ccccc23)cc1 ; CC(C)c1ccc(-c2c3ccccc3c(-c3ccc4c(c3)-c3cccc5cccc(c35)O4)c3ccccc23)cc1 ; CC(C)(C)c1ccc(-c2c3ccccc3c(-c3ccc4c(c3)-c3cccc5cccc(c35)O4)c3ccccc23)cc1 ; N#Cc1ccc(-c2c3ccccc3c(-c3ccc4c(c3)-c3cccc5cccc(c35)O4)c3ccccc23)cc1]

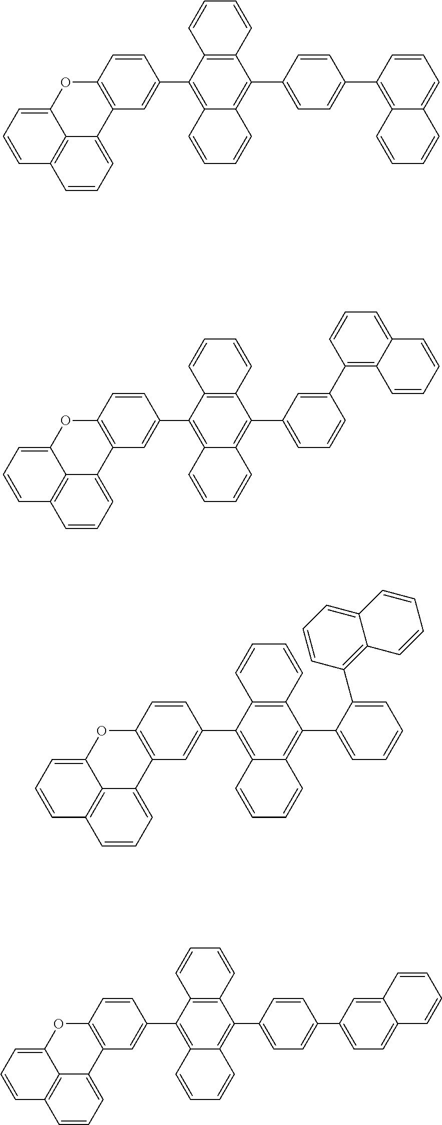 [SMILES: c1ccc2c(-c3ccc(-c4c5ccccc5c(-c5ccc6c(c5)-c5cccc7cccc(c57)O6)c5ccccc45)cc3)cccc2c1 ; c1cc(-c2cccc3ccccc23)cc(-c2c3ccccc3c(-c3ccc4c(c3)-c3cccc5cccc(c35)O4)c3ccccc23)c1 ; c1ccc(-c2c3ccccc3c(-c3ccc4c(c3)-c3cccc5cccc(c35)O4)c3ccccc23)c(-c2cccc3ccccc23)c1 ; c1ccc2cc(-c3ccc(-c4c5ccccc5c(-c5ccc6c(c5)-c5cccc7cccc(c57)O6)c5ccccc45)cc3)ccc2c1]

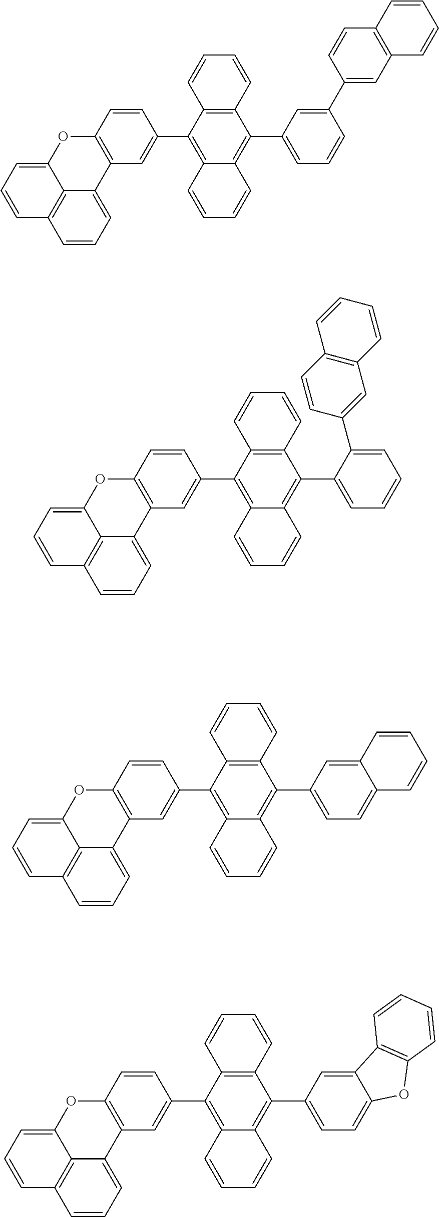 [SMILES: c1cc(-c2ccc3ccccc3c2)cc(-c2c3ccccc3c(-c3ccc4c(c3)-c3cccc5cccc(c35)O4)c3ccccc23)c1 ; c1ccc(-c2c3ccccc3c(-c3ccc4c(c3)-c3cccc5cccc(c35)O4)c3ccccc23)c(-c2ccc3ccccc3c2)c1 ; c1ccc2cc(-c3c4ccccc4c(-c4ccc5c(c4)-c4cccc6cccc(c46)O5)c4ccccc34)ccc2c1 ; c1cc2c3c(cccc3c1)-c1cc(-c3c4ccccc4c(-c4ccc5oc6ccccc6c5c4)c4ccccc34)ccc1O2]

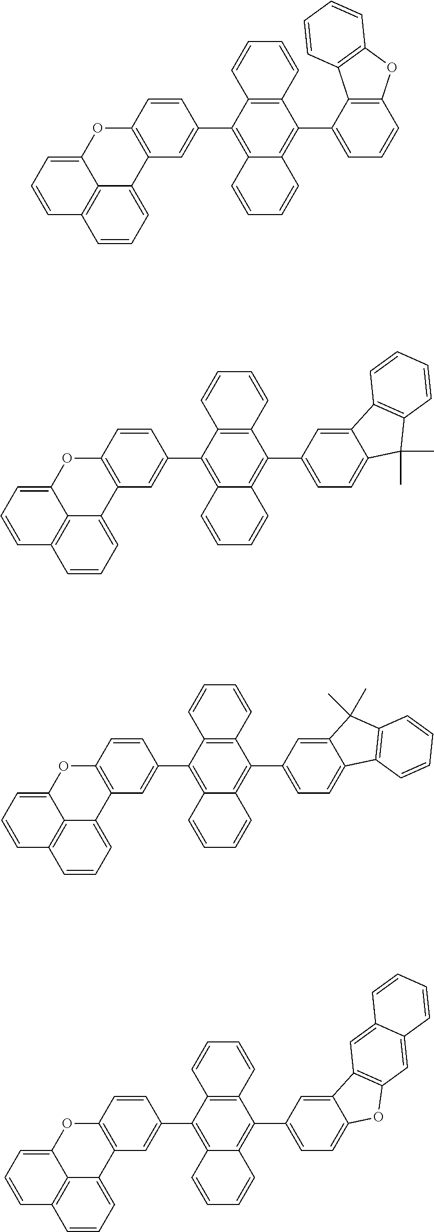 [SMILES: c1cc2c3c(cccc3c1)-c1cc(-c3c4ccccc4c(-c4cccc5oc6ccccc6c45)c4ccccc34)ccc1O2 ; CC1(C)c2ccccc2-c2cc(-c3c4ccccc4c(-c4ccc5c(c4)-c4cccc6cccc(c46)O5)c4ccccc34)ccc21 ; CC1(C)c2ccccc2-c2ccc(-c3c4ccccc4c(-c4ccc5c(c4)-c4cccc6cccc(c46)O5)c4ccccc34)cc21 ; c1ccc2cc3c(cc2c1)oc1ccc(-c2c4ccccc4c(-c4ccc5c(c4)-c4cccc6cccc(c46)O5)c4ccccc24)cc13]

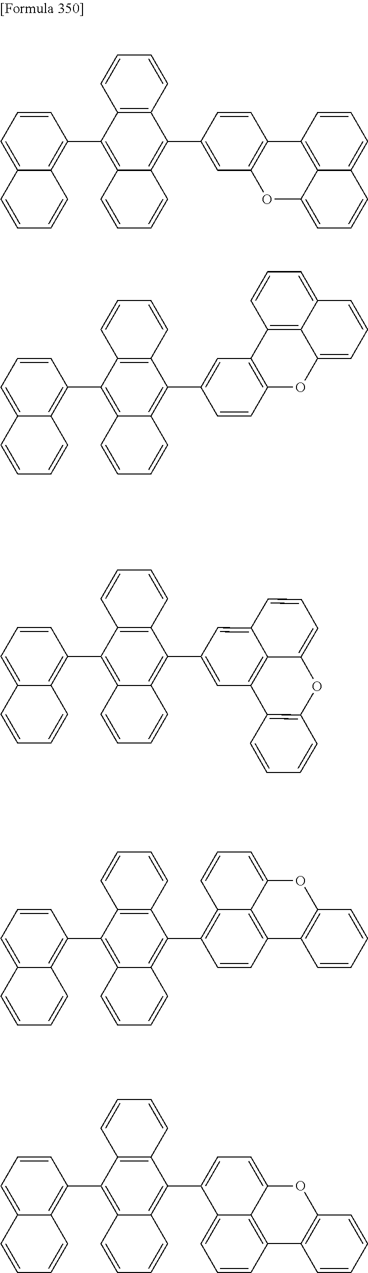 [SMILES: c1ccc2c(-c3c4ccccc4c(-c4ccc5c(c4)Oc4cccc6cccc-5c46)c4ccccc34)cccc2c1 ; c1ccc2c(-c3c4ccccc4c(-c4ccc5c(c4)-c4cccc6cccc(c46)O5)c4ccccc34)cccc2c1 ; c1ccc2c(c1)Oc1cccc3cc(-c4c5ccccc5c(-c5cccc6ccccc56)c5ccccc45)cc-2c13 ; c1ccc2c(c1)Oc1cccc3c(-c4c5ccccc5c(-c5cccc6ccccc56)c5ccccc45)ccc-2c13 ; c1ccc2c(c1)Oc1ccc(-c3c4ccccc4c(-c4cccc5ccccc45)c4ccccc34)c3cccc-2c13]

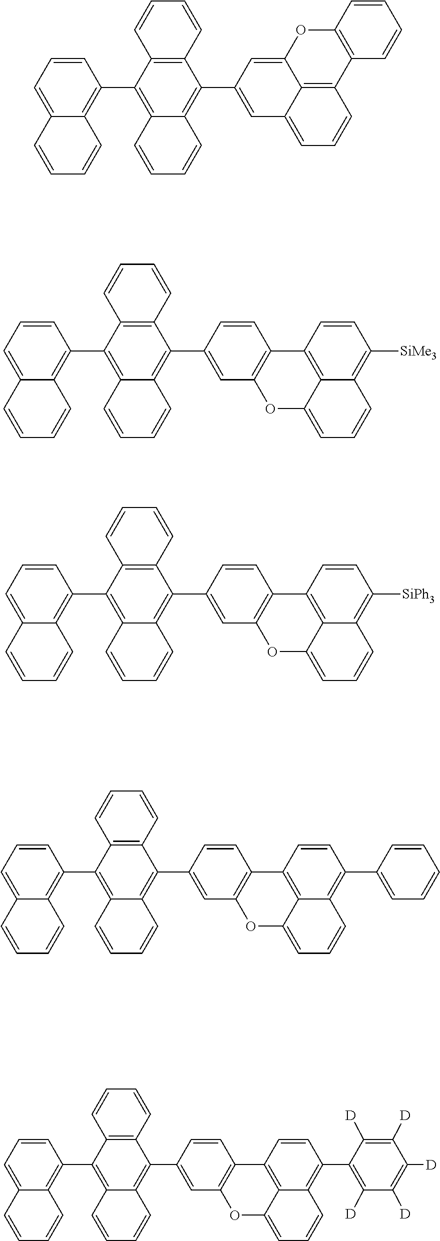

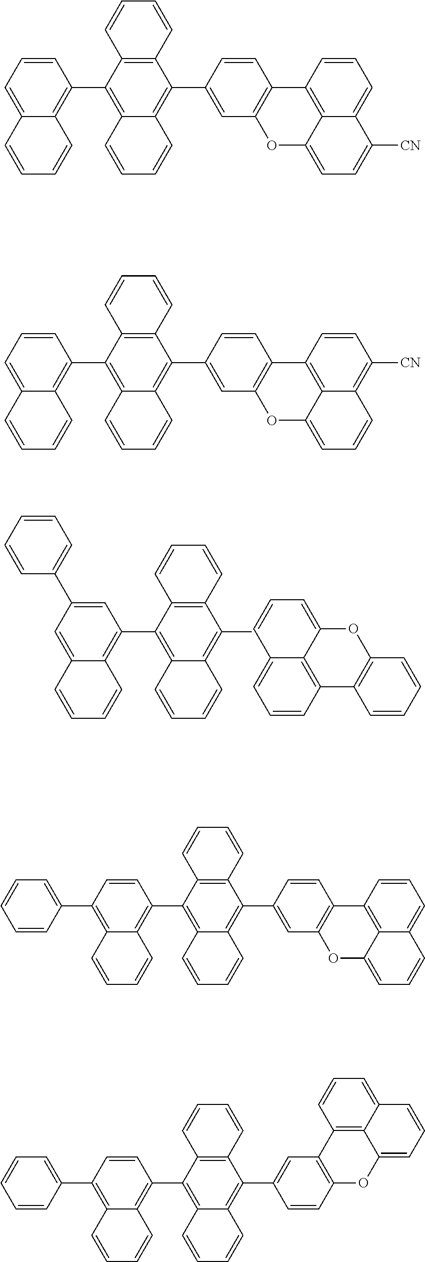

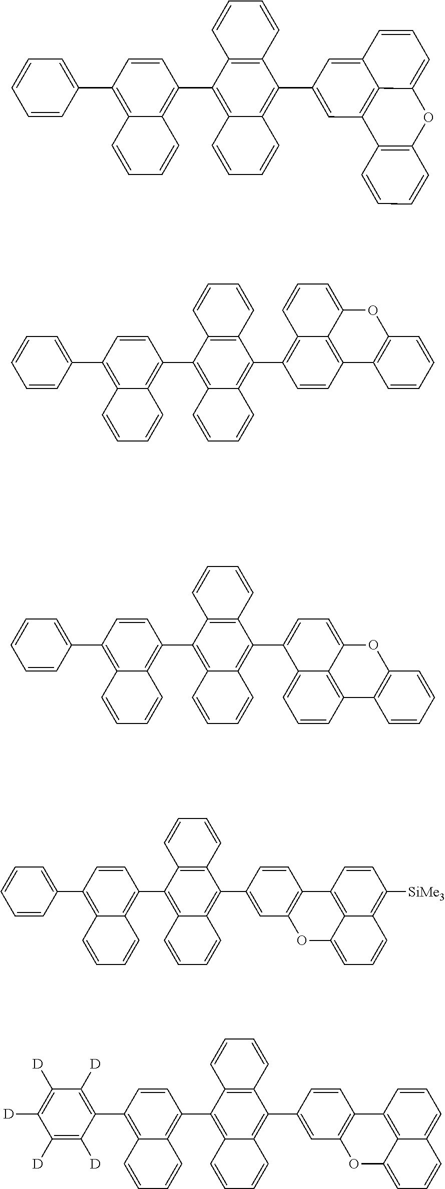 [SMILES: c1ccc(-c2ccc(-c3c4ccccc4c(-c4cc5c6c(cccc6c4)Oc4ccccc4-5)c4ccccc34)c3ccccc23)cc1 ; c1ccc(-c2ccc(-c3c4ccccc4c(-c4ccc5c6c(cccc46)Oc4ccccc4-5)c4ccccc34)c3ccccc23)cc1 ; c1ccc(-c2ccc(-c3c4ccccc4c(-c4ccc5c6c(cccc46)-c4ccccc4O5)c4ccccc34)c3ccccc23)cc1 ; C[Si](C)(C)c1ccc2c3c(cccc13)Oc1cc(-c3c4ccccc4c(-c4ccc(-c5ccccc5)c5ccccc45)c4ccccc34)ccc1-2 ; [2H]c1c([2H])c([2H])c(-c2ccc(-c3c4ccccc4c(-c4ccc5c(c4)Oc4cccc6cccc-5c46)c4ccccc34)c3ccccc23)c([2H])c1[2H]]

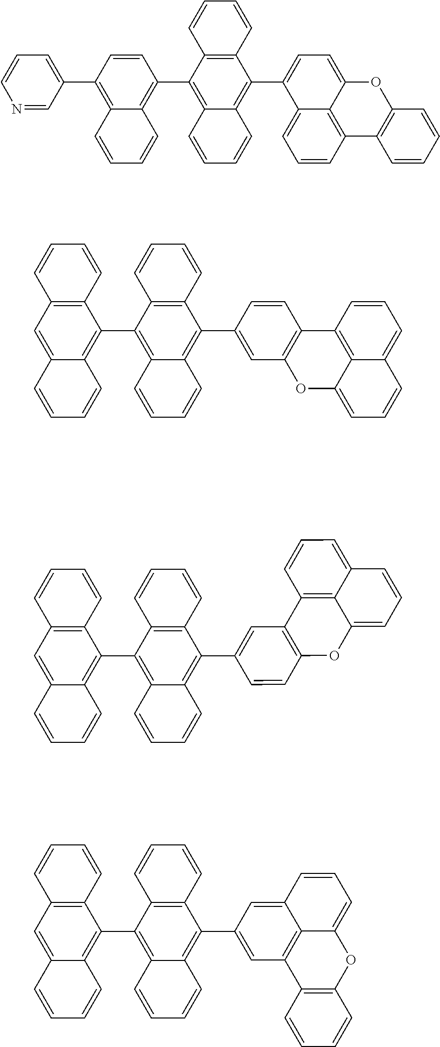 [SMILES: c1cncc(-c2ccc(-c3c4ccccc4c(-c4ccc5c6c(cccc46)-c4ccccc4O5)c4ccccc34)c3ccccc23)c1 ; c1ccc2c(-c3c4ccccc4c(-c4ccc5c(c4)Oc4cccc6cccc-5c46)c4ccccc34)c3ccccc3cc2c1 ; c1ccc2c(-c3c4ccccc4c(-c4ccc5c(c4)-c4cccc6cccc(c46)O5)c4ccccc34)c3ccccc3cc2c1 ; c1ccc2c(c1)Oc1cccc3cc(-c4c5ccccc5c(-c5c6ccccc6cc6ccccc56)c5ccccc45)cc-2c13]

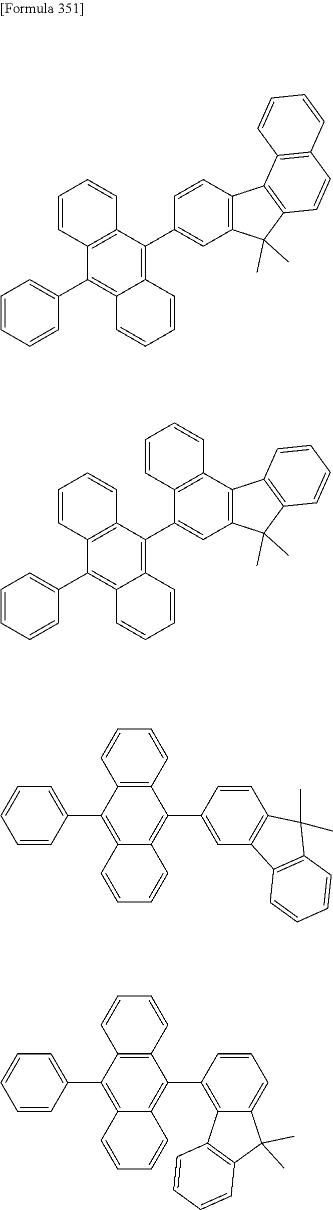

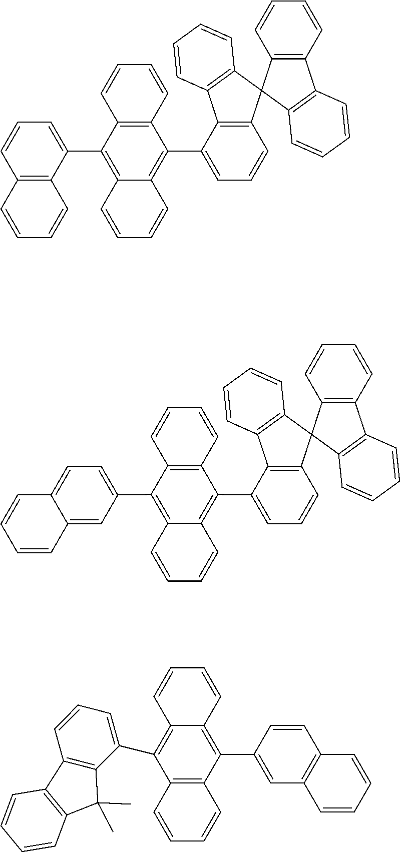

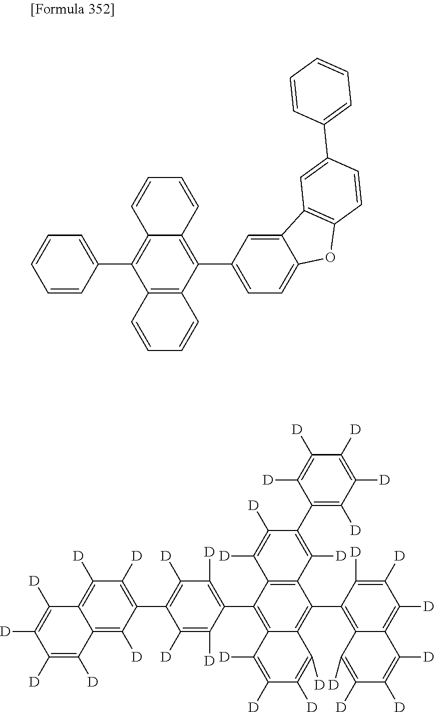 [SMILES: c1ccc(-c2ccc3oc4ccc(-c5c6ccccc6c(-c6ccccc6)c6ccccc56)cc4c3c2)cc1 ; [2H]c1c([2H])c([2H])c(-c2c([2H])c([2H])c3c(-c4c([2H])c([2H])c(-c5c([2H])c([2H])c6c([2H])c([2H])c([2H])c([2H])c6c5[2H])c([2H])c4[2H])c4c([2H])c([2H])c([2H])c([2H])c4c(-c4c([2H])c([2H])c([2H])c5c([2H])c([2H])c([2H])c([2H])c45)c3c2[2H])c([2H])c1[2H]]

### Third Compound and Fourth Compound

The third compound and the fourth compound are each independently at least one compound selected from the group consisting of a compound represented by a formula (3) below, a compound represented by a formula (4) below, a compound represented by a formula (5) below, a compound represented by a formula (6) below, a compound represented by a formula (7) below, a compound represented by a formula (8) below, a compound represented by a formula (9) below, and a compound represented by a formula (10) below.

Compound Represented by Formula (3)

The compound represented by the formula (3) will be described.

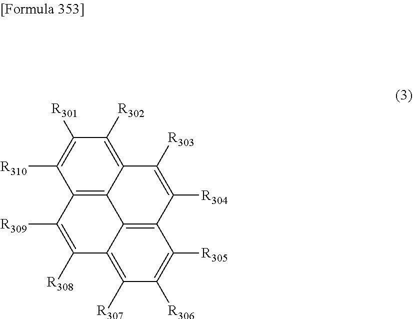

In the formula (3):
at least one combination of adjacent two or more of R₃₀₁ to R₃₁₀ are mutually bonded to form a substituted or unsubstituted monocyclic ring, mutually bonded to form a substituted or unsubstituted fused ring, or not mutually bonded;
at least one of R₃₀₁ to R₃₁₀ is a monovalent group represented by a formula (31) below; and
R₃₀₁ to R₃₁₀ forming neither the substituted or unsubstituted monocyclic ring nor the substituted or unsubstituted fused ring and not being a monovalent group represented by the formula (31) below are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

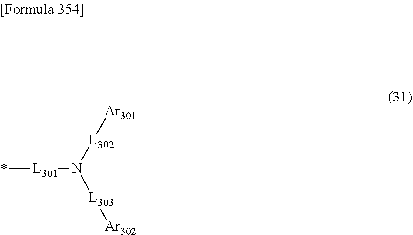

In the formula (31):
Ar₃₀₁ and Ar₃₀₂ are each independently a substituted or unsubstituted aryl group having 6 to 50 ring carbon atoms, or a substituted or unsubstituted heterocyclic group having 5 to 50 ring atoms;
L₃₀₁ to L₃₀₃ are each independently a single bond, a substituted or unsubstituted arylene group having 6 to 30 ring carbon atoms, or a substituted or unsubstituted divalent heterocyclic group having 5 to 30 ring atoms; and
* represents a bonding position in a pyrene ring in the formula (3).

In the third and fourth compounds, R₉₀₁, R₉₀₂, R₉₀₃, R₉₀₄, R₉₀₅, R₉₀₆, and R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different; and
when a plurality of R₉₀₇ are present, the plurality of R₉₀₇ are mutually the same or different.

In the formula (3), two of R₃₀₁ to R₃₁₀ are each preferably a group represented by the formula (31).

In an exemplary embodiment, the compound represented by the formula (3) is a compound represented by a formula (33) below.

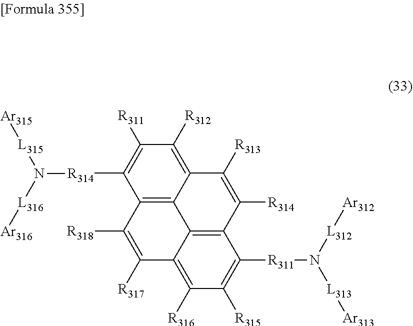

In the formula (33):
R₃₁₁ to R₃₁₈ each independently represent the same as R₃₀₁ to R₃₁₀ in the formula (3) that are not the monovalent group represented by the formula (31);
L₃₁₁ to L₃₁₆ are each independently a single bond, a substituted or unsubstituted arylene group having 6 to 30 ring carbon atoms, or a substituted or unsubstituted divalent heterocyclic group having 5 to 30 ring atoms;
Ar₃₁₂, Ar₃₁₃, Ar₃₁₅, and Ar₃₁₆ are each independently a substituted or unsubstituted aryl group having 6 to 50 ring carbon atoms, or a substituted or unsubstituted heterocyclic group having 5 to 50 ring atoms.

In the formula (31), L₃₀₁ is preferably a single bond, and L₃₀₂ and L₃₀₃ are each preferably a single bond.

In an exemplary embodiment, the compound represented by the formula (3) is represented by a formula (34) or a formula (35) below.

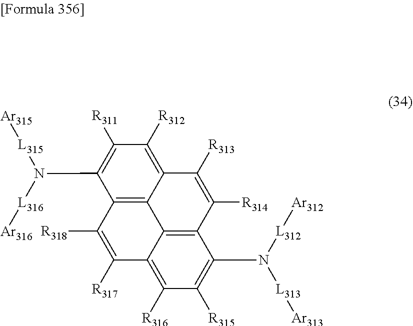

In the formula (34):
R₃₁₁ to R₃₁₈ each independently represent the same as R₃₀₁ to R₃₁₀ in the formula (3) that are not the monovalent group represented by the formula (31);
L₃₁₂, L₃₁₃, L₃₁₅ and L₃₁₆ each independently represent the same as L₃₁₂, L₃₁₃, L₃₁₅ and L₃₁₆ in the formula (33); and
Ar₃₁₂, Ar₃₁₃, Ar₃₁₅ and Ar₃₁₆ each independently represent the same as Ar₃₁₂, Ar₃₁₃, Ar₃₁₅ and Ar₃₁₆ in the formula (33).

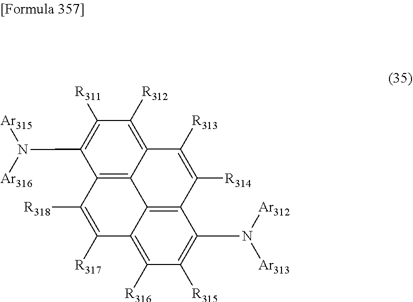

In the formula (35):
R₃₁₁ to R₃₁₈ each independently represent the same as R₃₀₁ to R₃₁₀ in the formula (3) that are not the monovalent group represented by the formula (31); and
Ar₃₁₂, Ar₃₁₃, Ar₃₁₅ and Ar₃₁₆ each independently represent the same as Ar₃₁₂, Ar₃₁₃, Ar₃₁₅ and Ar₃₁₆ in the formula (33).

In the formula (31), at least one of Ar₃₀₁ or Ar₃₀₂ is preferably a group represented by a formula (36) below.

In the formulae (33) to (35), at least one of Ar₃₁₂ or Ar₃₁₃ is preferably a group represented by the formula (36) below.

In the formulae (33) to (35), at least one of Ar₃₁₅ or Ar₃₁₆ is preferably a group represented by the formula (36) below.

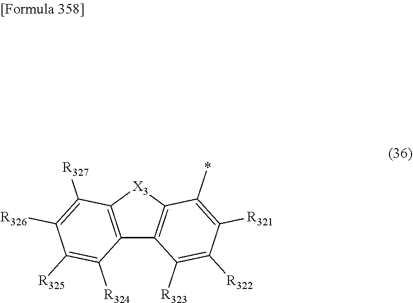

In the formula (36):
X₃ represents an oxygen atom or a sulfur atom;
at least one combination of adjacent two or more of R₃₂₁ to R₃₂₇ are mutually bonded to form a substituted or unsubstituted monocyclic ring, mutually bonded to form a substituted or unsubstituted fused ring, or not mutually bonded;
R₃₂₁ to R₃₂₇ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms; and
* represents a bonding position to L₃₀₂, L₃₀₃, L₃₁₂, L₃₁₃, L₃₁₅ or L₃₁₆.

X₃ is preferably an oxygen atom.

At least one of R₃₂₁ to R₃₂₇ is preferably a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In the formula (31), it is preferable that Ar₃₀₁ is a group represented by the formula (36) and Ar₃₀₂ is a substituted or unsubstituted aryl group having 6 to 50 ring carbon atoms.

In the formulae (33) to (35), it is preferable that Ar₃₁₂ is a group represented by the formula (36) and Ar₃₁₃ is a substituted or unsubstituted aryl group having 6 to 50 ring carbon atoms.

In the formulae (33) to (35), it is preferable that Ar₃₁₅ is a group represented by the formula (36) and Ar₃₁₆ is a substituted or unsubstituted aryl group having 6 to 50 ring carbon atoms.

In an exemplary embodiment, the compound represented by the formula (3) is represented by a formula (37) below.

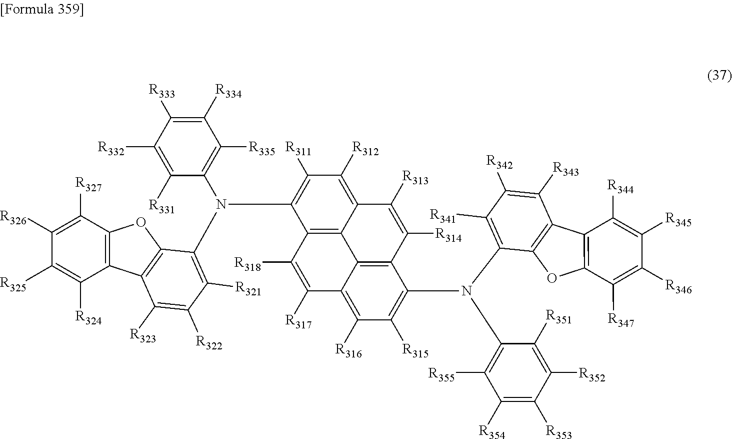

In the formula (37):
R₃₁₁ to R₃₁₅ each independently represent the same as R₃₁ to R₃₁ in the formula (3) that are not the monovalent group represented by the formula (31);
at least one combination of adjacent two or more of R₃₂₁ to R₃₂₇ are mutually bonded to form a substituted or unsubstituted monocyclic ring, mutually bonded to form a substituted or unsubstituted fused ring, or not mutually bonded;
at least one combination of adjacent two or more of R₃₄₁ to R₃₄₇ are mutually bonded to form a substituted or unsubstituted monocyclic ring, mutually bonded to form a substituted or unsubstituted fused ring, or not mutually bonded;
R₃₂₁ to R₃₂₇ and R₃₄₁ to R₃₄₇ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms,
R₃₃₁ to R₃₃₅ and R₃₅₁ to R₃₅₅ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

Specific examples of the compound represented by the formula (3) include compounds shown below.

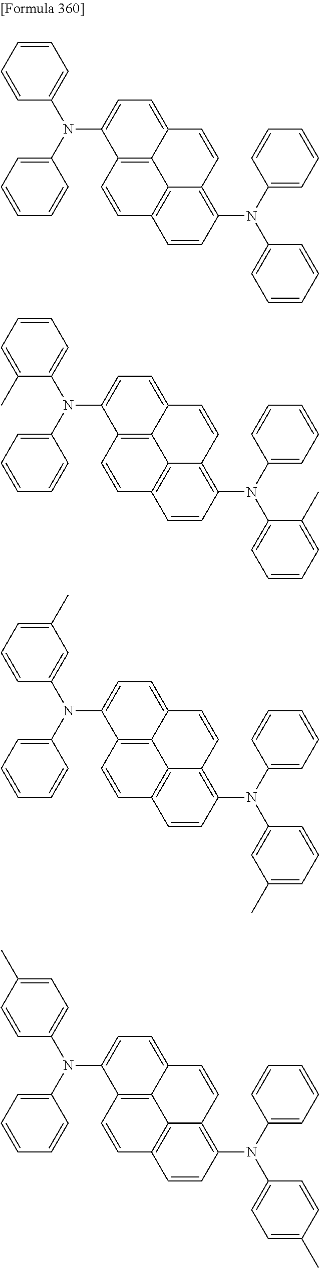 [SMILES: c1ccc(N(c2ccccc2)c2ccc3ccc4c(N(c5ccccc5)c5ccccc5)ccc5ccc2c3c54)cc1 ; Cc1ccccc1N(c1ccccc1)c1ccc2ccc3c(N(c4ccccc4)c4ccccc4C)ccc4ccc1c2c43 ; Cc1cccc(N(c2ccccc2)c2ccc3ccc4c(N(c5ccccc5)c5cccc(C)c5)ccc5ccc2c3c54)c1 ; Cc1ccc(N(c2ccccc2)c2ccc3ccc4c(N(c5ccccc5)c5ccc(C)cc5)ccc5ccc2c3c54)cc1]

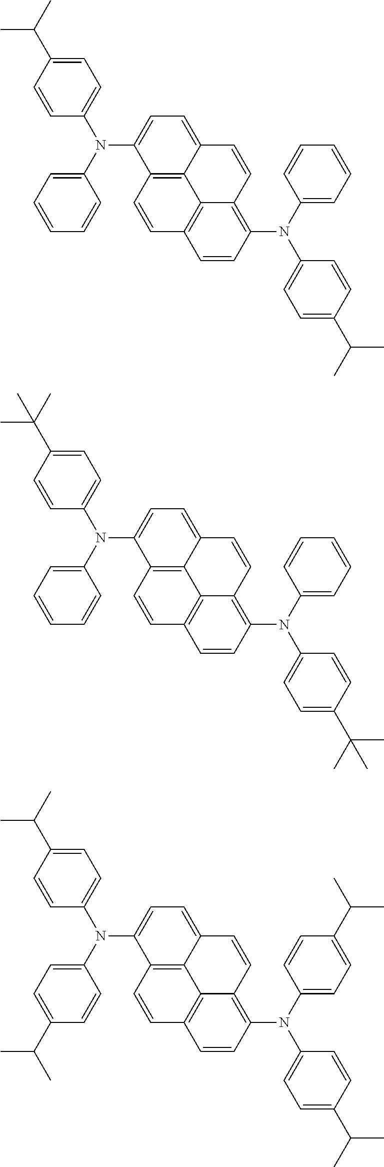

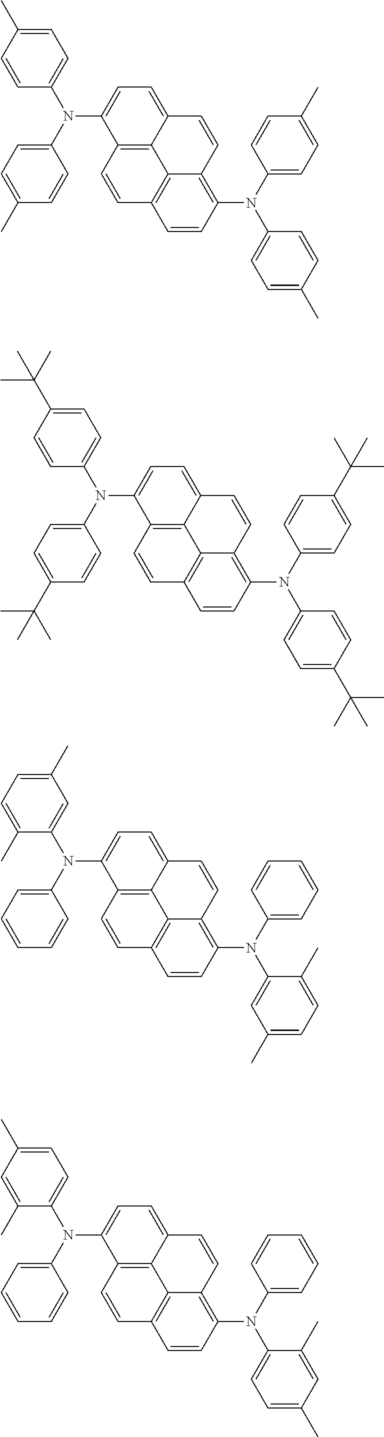

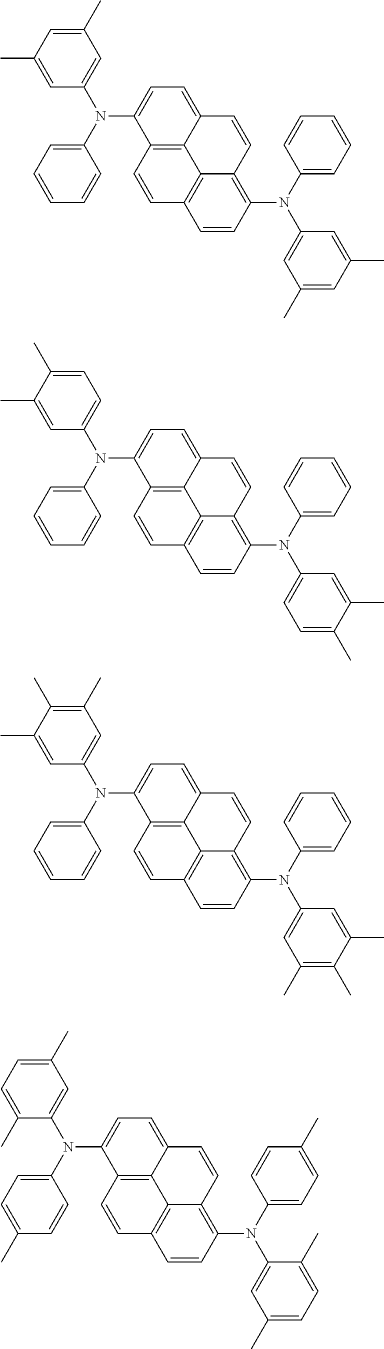

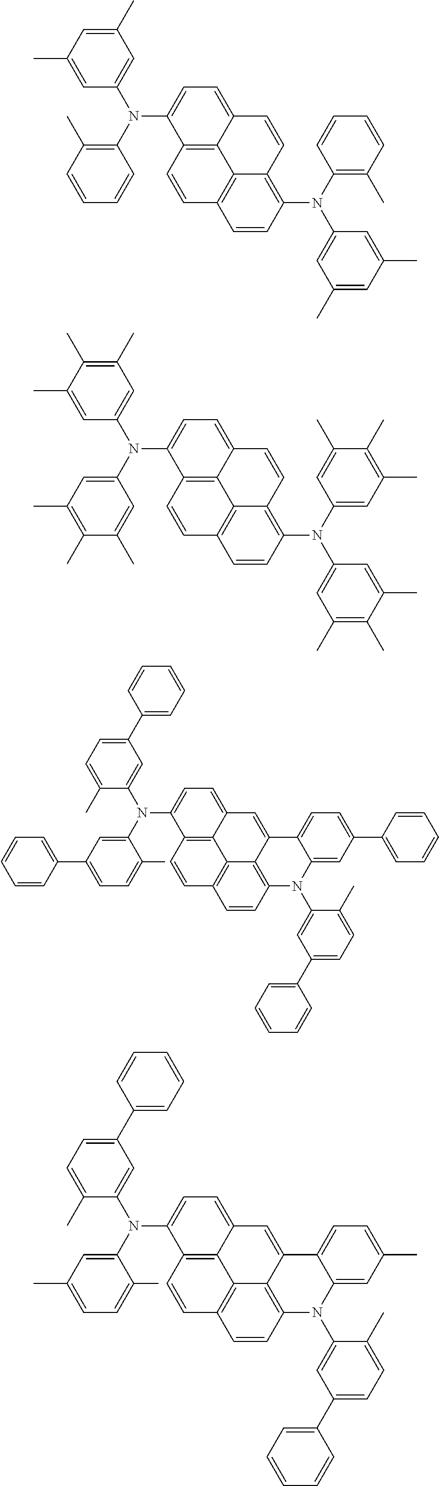

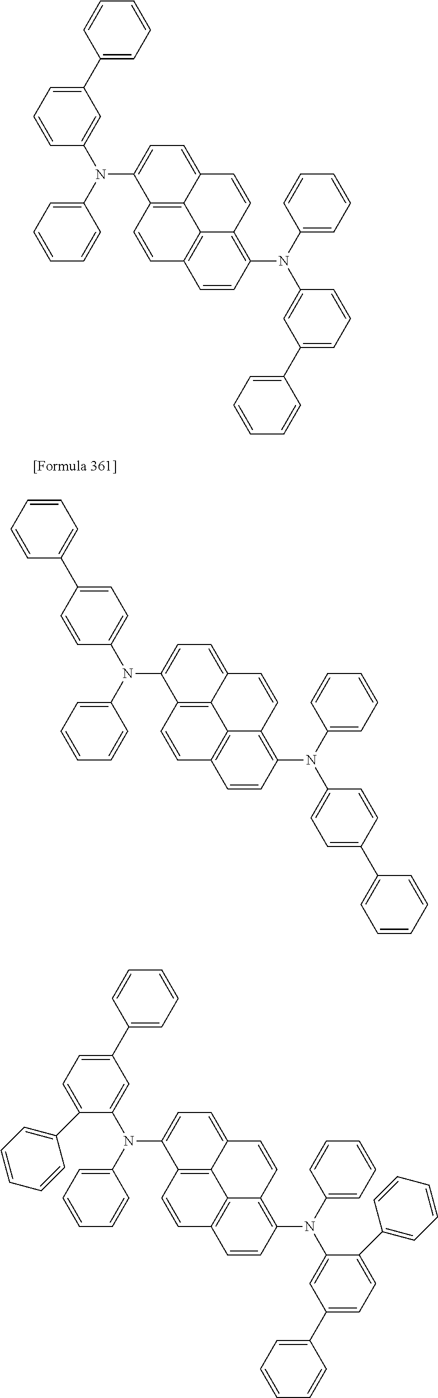 [SMILES: c1ccc(-c2cccc(N(c3ccccc3)c3ccc4ccc5c(N(c6ccccc6)c6cccc(-c7ccccc7)c6)ccc6ccc3c4c65)c2)cc1 ; c1ccc(-c2ccc(N(c3ccccc3)c3ccc4ccc5c(N(c6ccccc6)c6ccc(-c7ccccc7)cc6)ccc6ccc3c4c65)cc2)cc1 ; c1ccc(-c2ccc(-c3ccccc3)c(N(c3ccccc3)c3ccc4ccc5c(N(c6ccccc6)c6cc(-c7ccccc7)ccc6-c6ccccc6)ccc6ccc3c4c65)c2)cc1]

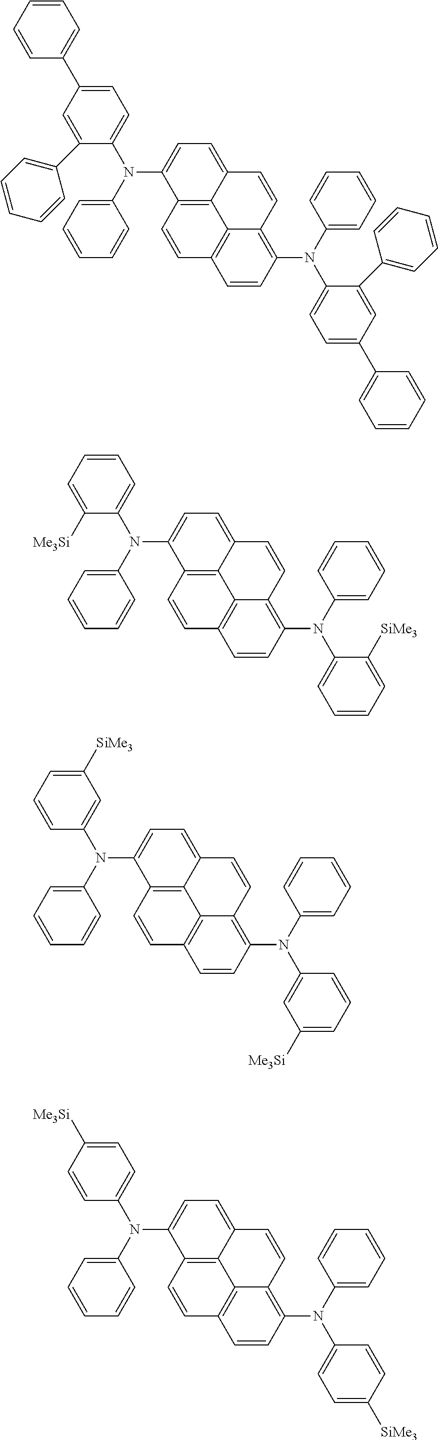 [SMILES: c1ccc(-c2ccc(N(c3ccccc3)c3ccc4ccc5c(N(c6ccccc6)c6ccc(-c7ccccc7)cc6-c6ccccc6)ccc6ccc3c4c65)c(-c3ccccc3)c2)cc1 ; C[Si](C)(C)c1ccccc1N(c1ccccc1)c1ccc2ccc3c(N(c4ccccc4)c4ccccc4[Si](C)(C)C)ccc4ccc1c2c43 ; C[Si](C)(C)c1cccc(N(c2ccccc2)c2ccc3ccc4c(N(c5ccccc5)c5cccc([Si](C)(C)C)c5)ccc5ccc2c3c54)c1 ; C[Si](C)(C)c1ccc(N(c2ccccc2)c2ccc3ccc4c(N(c5ccccc5)c5ccc([Si](C)(C)C)cc5)ccc5ccc2c3c54)cc1]

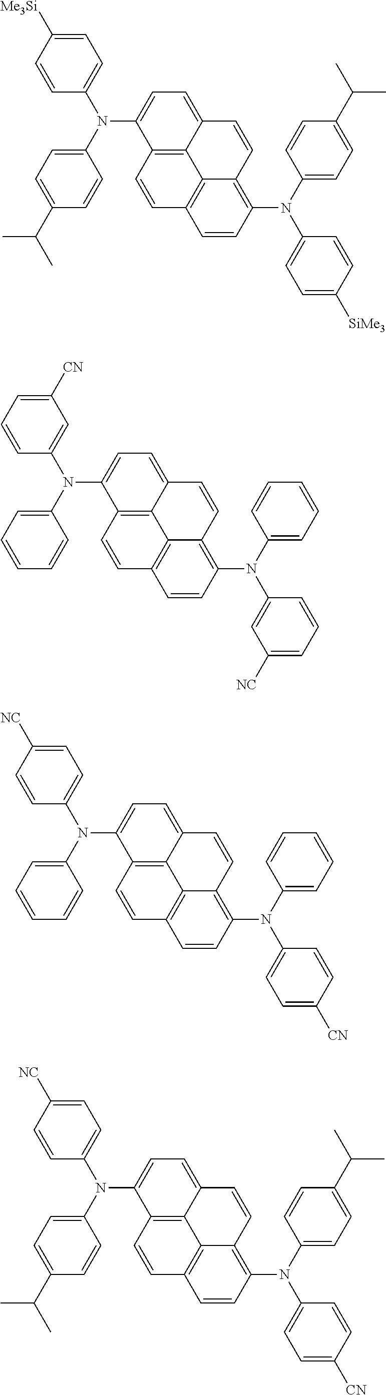

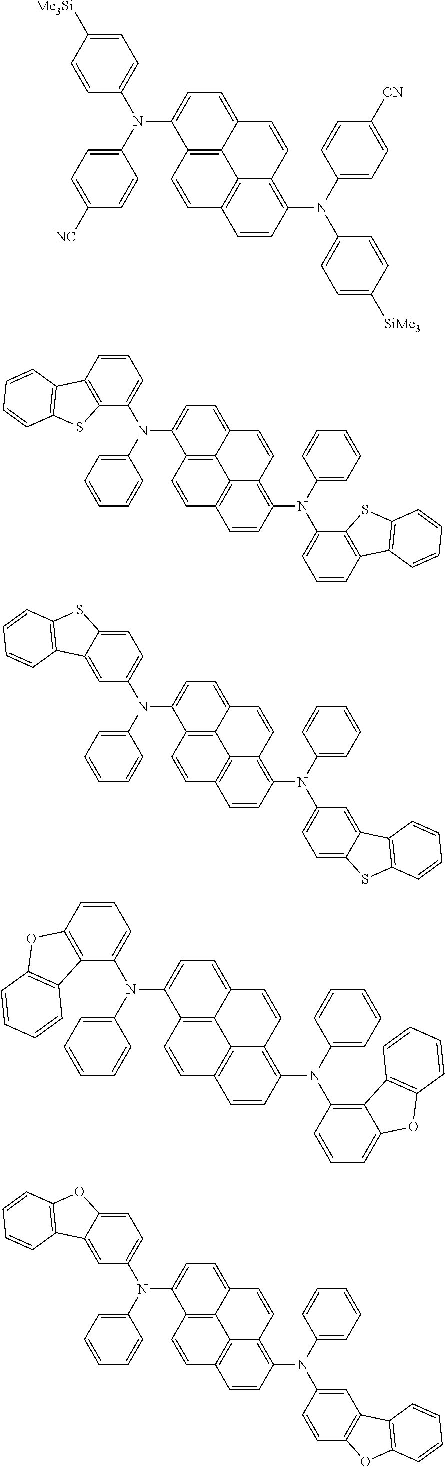 [SMILES: C[Si](C)(C)c1ccc(N(c2ccc(C#N)cc2)c2ccc3ccc4c(N(c5ccc(C#N)cc5)c5ccc([Si](C)(C)C)cc5)ccc5ccc2c3c54)cc1 ; c1ccc(N(c2ccc3ccc4c(N(c5ccccc5)c5cccc6c5sc5ccccc56)ccc5ccc2c3c54)c2cccc3c2sc2ccccc23)cc1 ; c1ccc(N(c2ccc3sc4ccccc4c3c2)c2ccc3ccc4c(N(c5ccccc5)c5ccc6sc7ccccc7c6c5)ccc5ccc2c3c54)cc1 ; c1ccc(N(c2ccc3ccc4c(N(c5ccccc5)c5cccc6oc7ccccc7c56)ccc5ccc2c3c54)c2cccc3oc4ccccc4c23)cc1 ; c1ccc(N(c2ccc3oc4ccccc4c3c2)c2ccc3ccc4c(N(c5ccccc5)c5ccc6oc7ccccc7c6c5)ccc5ccc2c3c54)cc1]

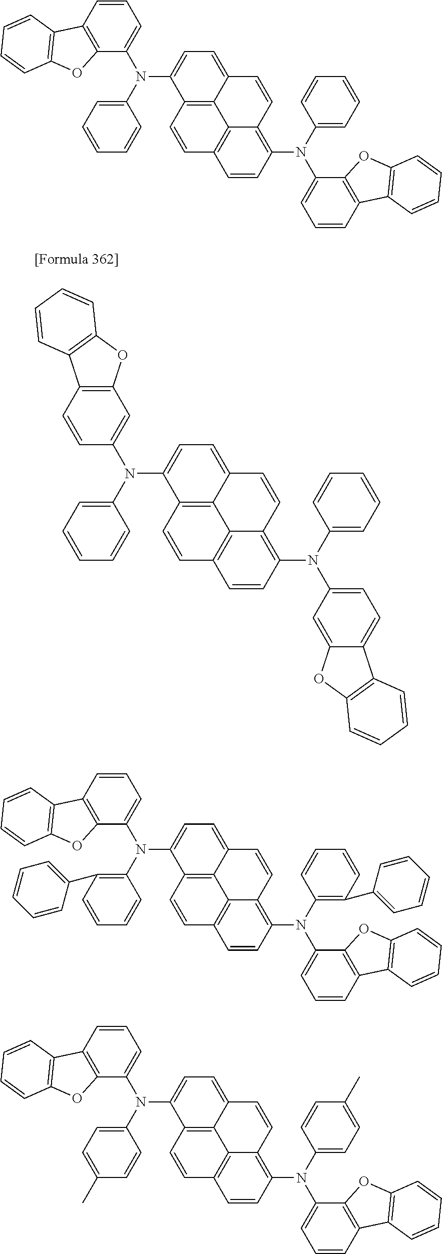 [SMILES: c1ccc(N(c2ccc3ccc4c(N(c5ccccc5)c5cccc6c5oc5ccccc56)ccc5ccc2c3c54)c2cccc3c2oc2ccccc23)cc1 ; c1ccc(N(c2ccc3c(c2)oc2ccccc23)c2ccc3ccc4c(N(c5ccccc5)c5ccc6c(c5)oc5ccccc56)ccc5ccc2c3c54)cc1 ; c1ccc(-c2ccccc2N(c2ccc3ccc4c(N(c5ccccc5-c5ccccc5)c5cccc6c5oc5ccccc56)ccc5ccc2c3c54)c2cccc3c2oc2ccccc23)cc1 ; Cc1ccc(N(c2ccc3ccc4c(N(c5ccc(C)cc5)c5cccc6c5oc5ccccc56)ccc5ccc2c3c54)c2cccc3c2oc2ccccc23)cc1]

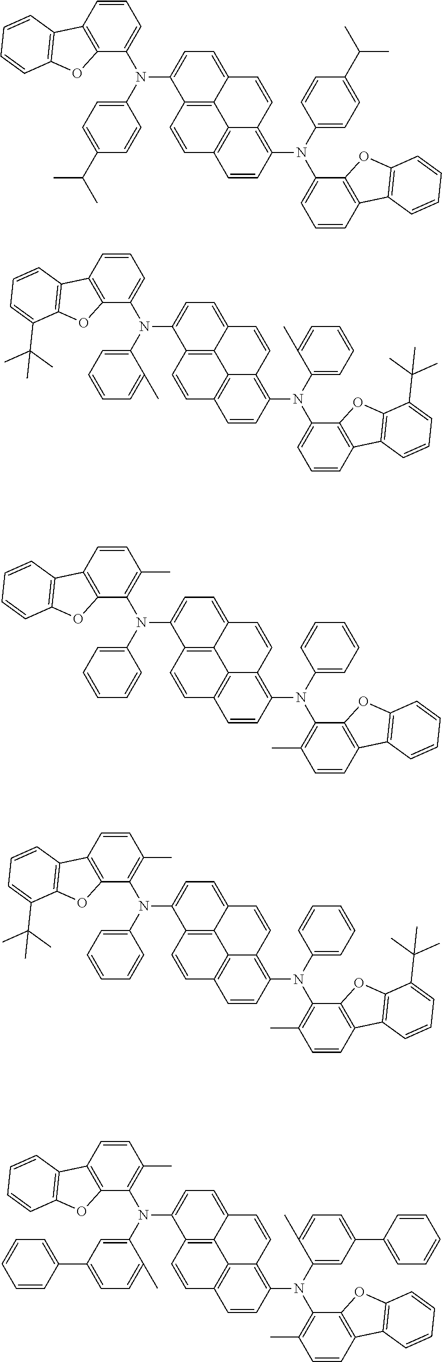

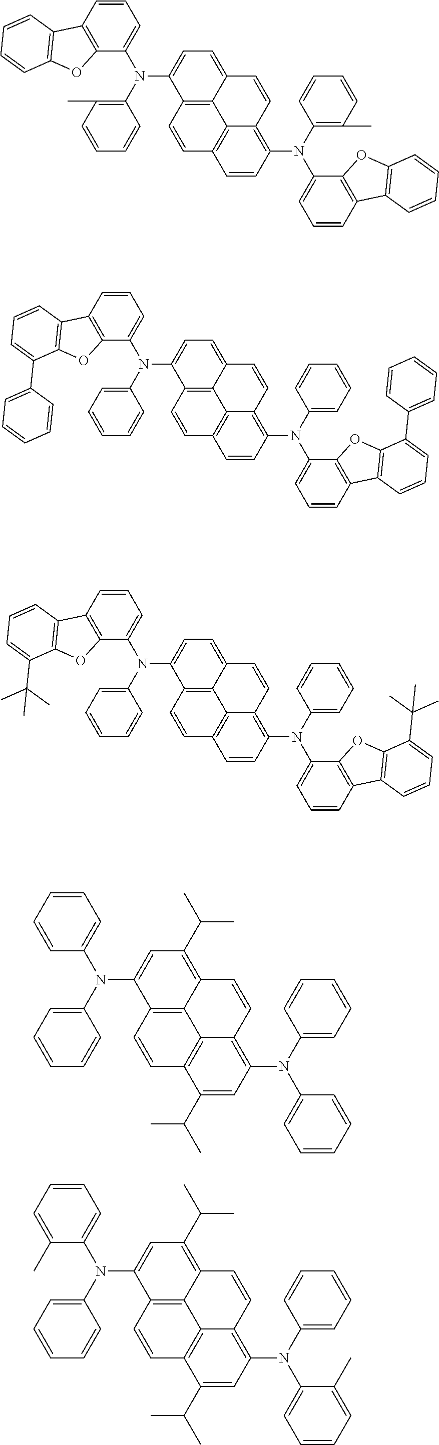

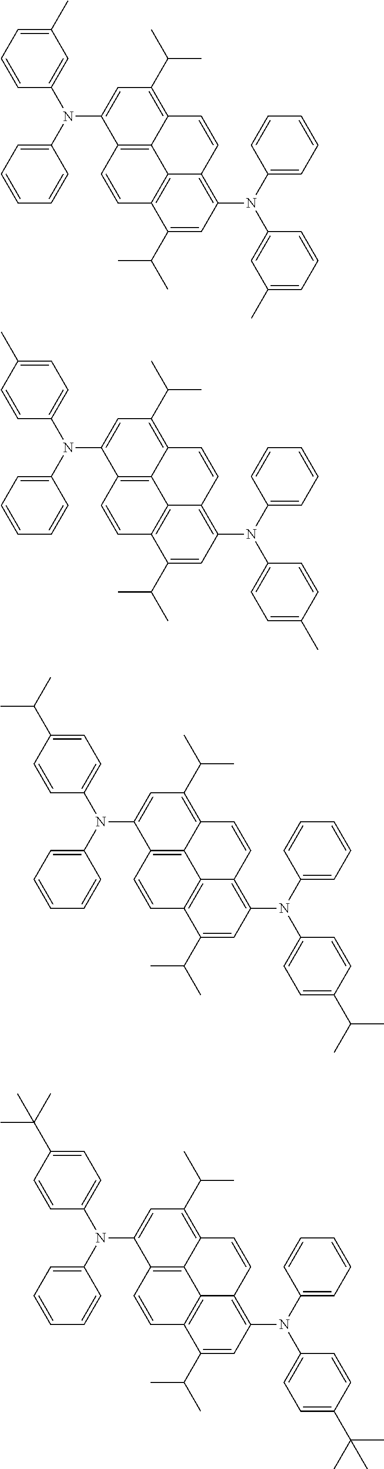

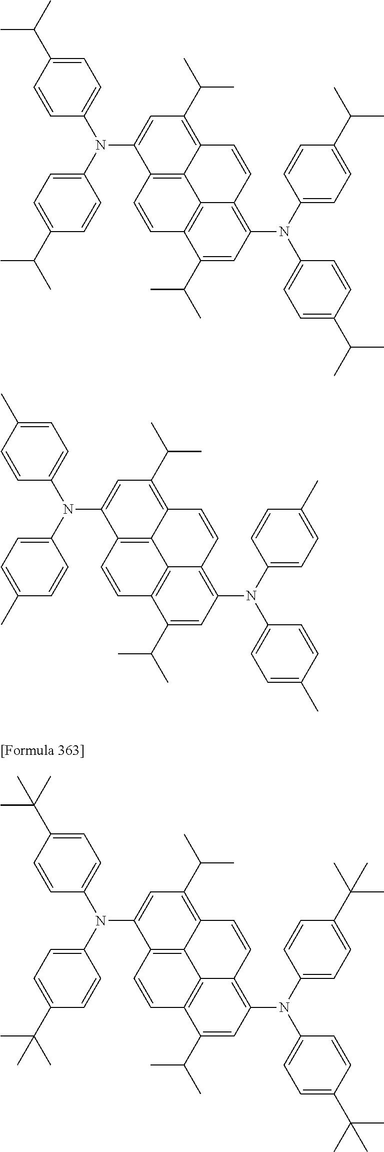

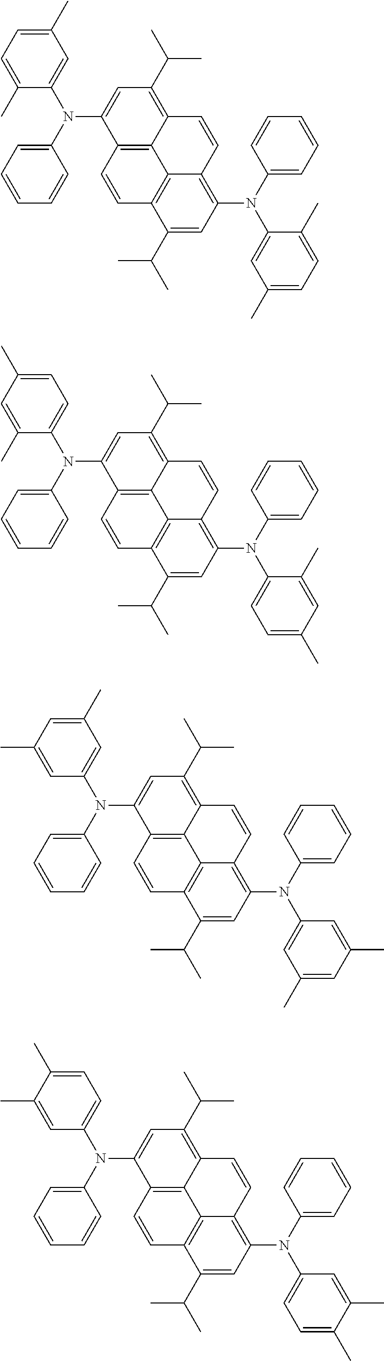

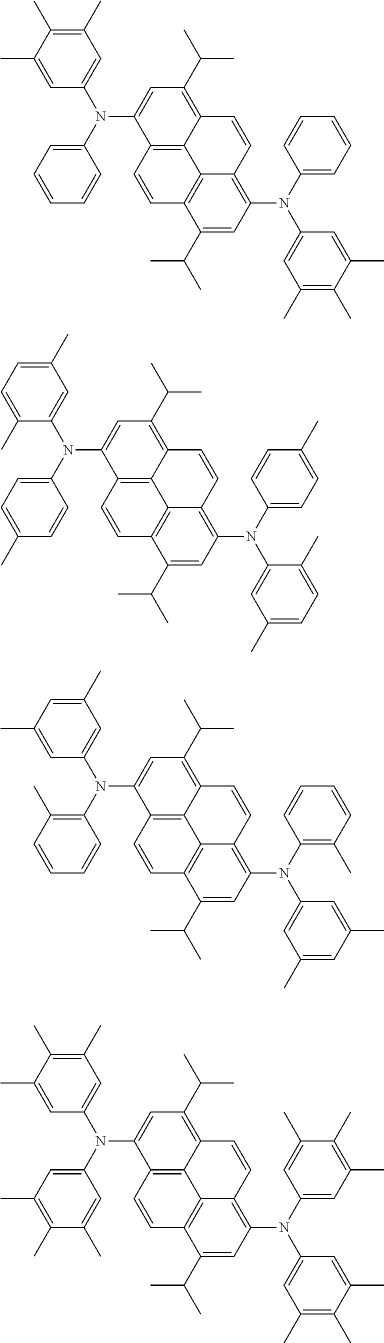

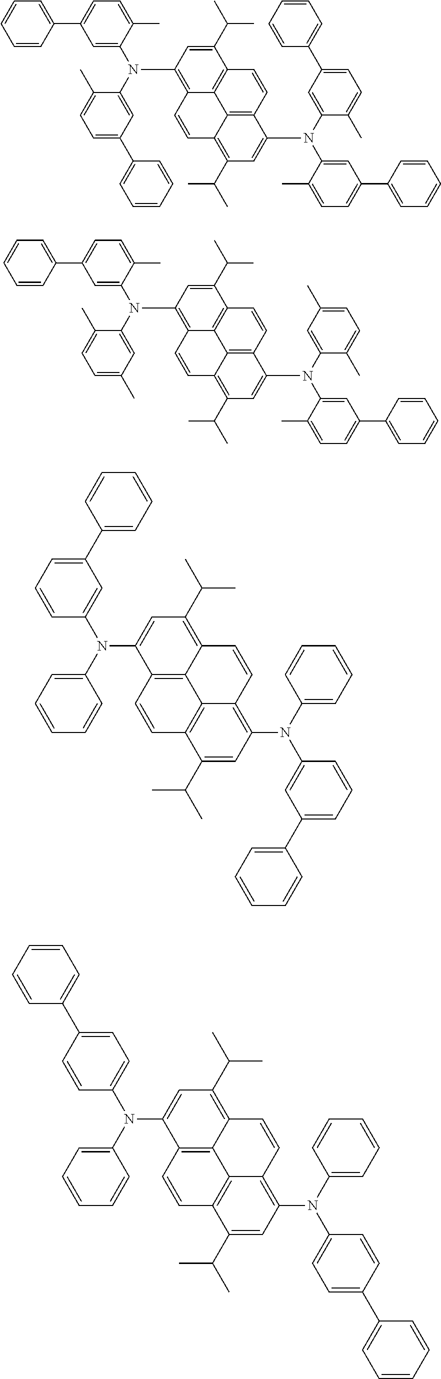

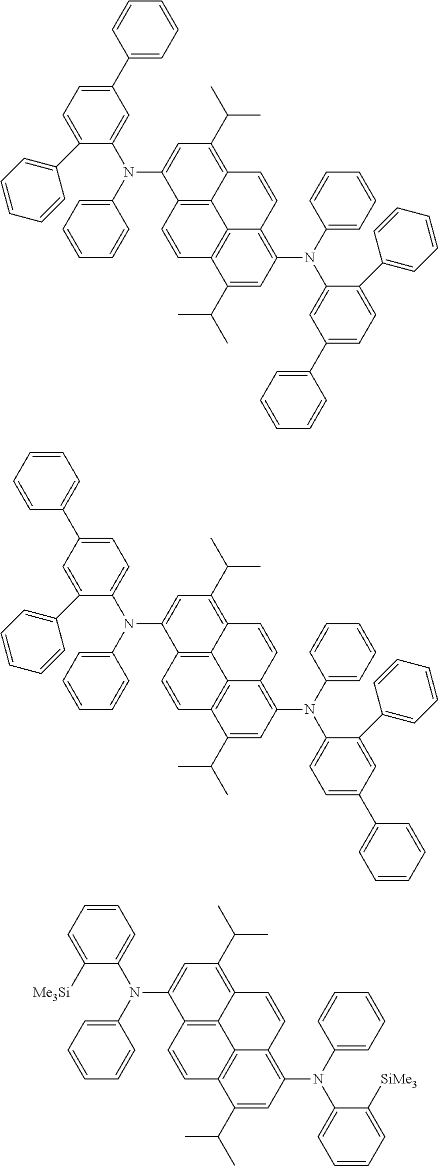 [SMILES: CC(C)c1cc(N(c2ccccc2)c2cc(-c3ccccc3)ccc2-c2ccccc2)c2ccc3c(C(C)C)cc(N(c4ccccc4)c4cc(-c5ccccc5)ccc4-c4ccccc4)c4ccc1c2c34 ; CC(C)c1cc(N(c2ccccc2)c2ccc(-c3ccccc3)cc2-c2ccccc2)c2ccc3c(C(C)C)cc(N(c4ccccc4)c4ccc(-c5ccccc5)cc4-c4ccccc4)c4ccc1c2c34 ; CC(C)c1cc(N(c2ccccc2)c2ccccc2[Si](C)(C)C)c2ccc3c(C(C)C)cc(N(c4ccccc4)c4ccccc4[Si](C)(C)C)c4ccc1c2c34]

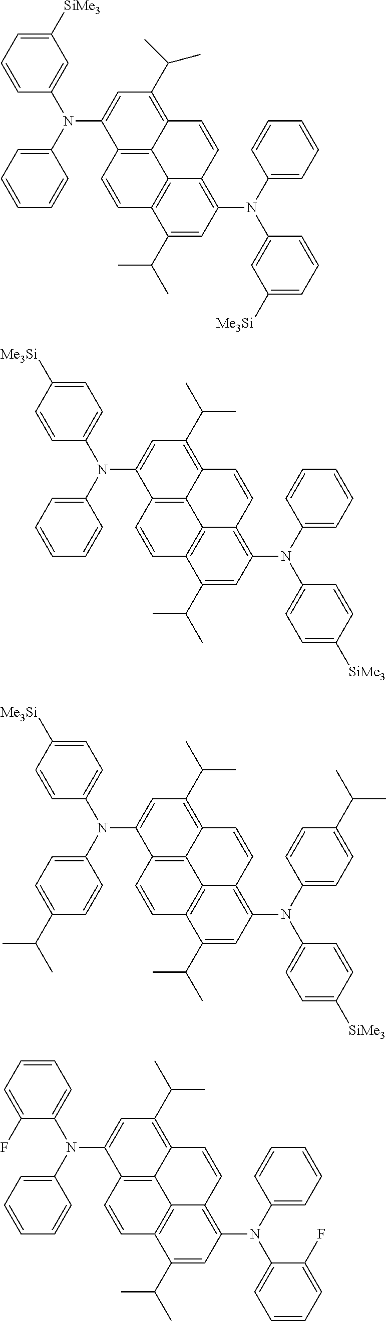 [SMILES: CC(C)c1cc(N(c2ccccc2)c2cccc([Si](C)(C)C)c2)c2ccc3c(C(C)C)cc(N(c4ccccc4)c4cccc([Si](C)(C)C)c4)c4ccc1c2c34 ; CC(C)c1cc(N(c2ccccc2)c2ccc([Si](C)(C)C)cc2)c2ccc3c(C(C)C)cc(N(c4ccccc4)c4ccc([Si](C)(C)C)cc4)c4ccc1c2c34 ; CC(C)c1ccc(N(c2ccc([Si](C)(C)C)cc2)c2cc(C(C)C)c3ccc4c(N(c5ccc(C(C)C)cc5)c5ccc([Si](C)(C)C)cc5)cc(C(C)C)c5ccc2c3c54)cc1 ; CC(C)c1cc(N(c2ccccc2)c2ccccc2F)c2ccc3c(C(C)C)cc(N(c4ccccc4)c4ccccc4F)c4ccc1c2c34]

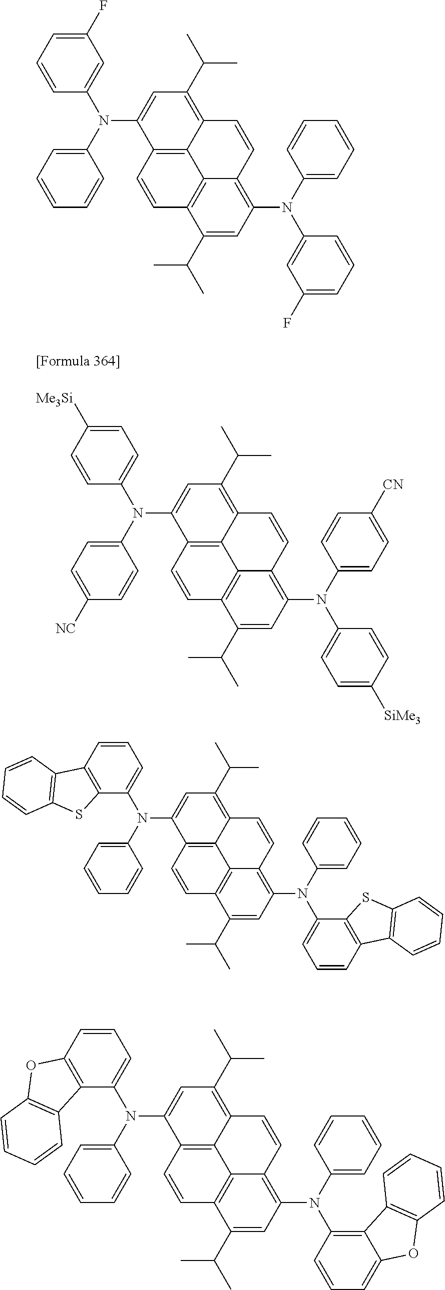 [SMILES: CC(C)c1cc(N(c2ccccc2)c2cccc(F)c2)c2ccc3c(C(C)C)cc(N(c4ccccc4)c4cccc(F)c4)c4ccc1c2c34 ; CC(C)c1cc(N(c2ccc(C#N)cc2)c2ccc([Si](C)(C)C)cc2)c2ccc3c(C(C)C)cc(N(c4ccc(C#N)cc4)c4ccc([Si](C)(C)C)cc4)c4ccc1c2c34 ; CC(C)c1cc(N(c2ccccc2)c2cccc3c2sc2ccccc23)c2ccc3c(C(C)C)cc(N(c4ccccc4)c4cccc5c4sc4ccccc45)c4ccc1c2c34 ; CC(C)c1cc(N(c2ccccc2)c2cccc3oc4ccccc4c23)c2ccc3c(C(C)C)cc(N(c4ccccc4)c4cccc5oc6ccccc6c45)c4ccc1c2c34]

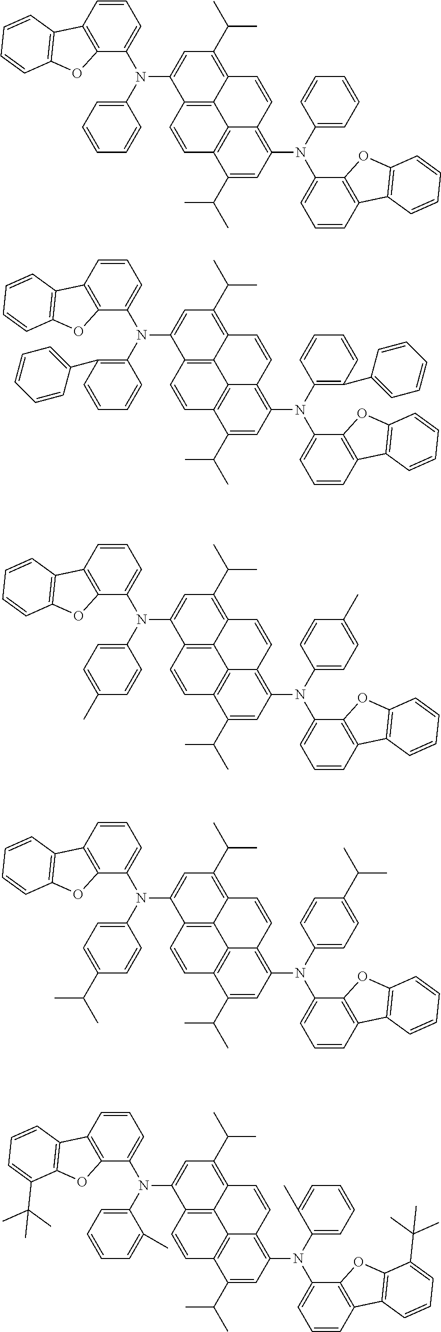

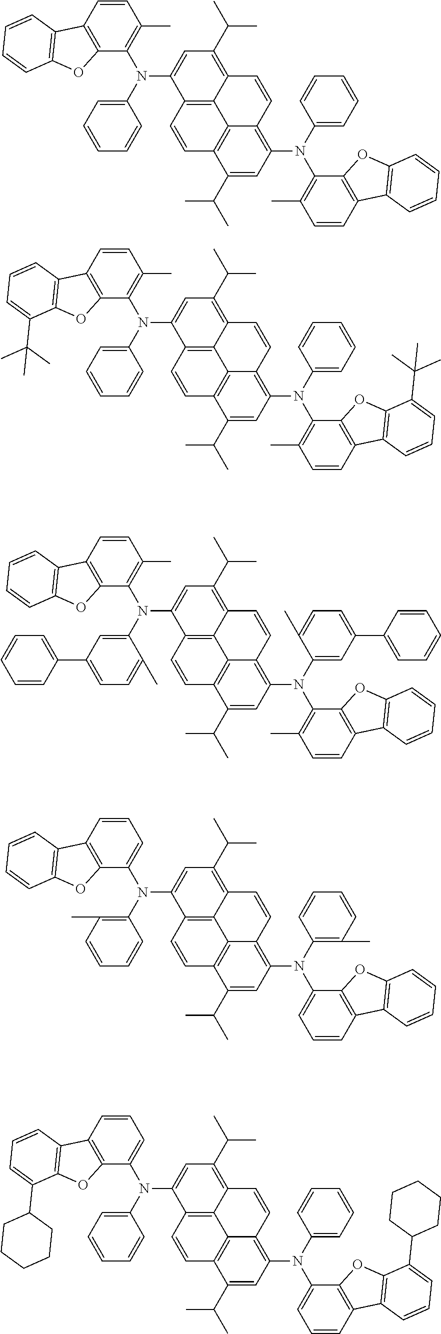

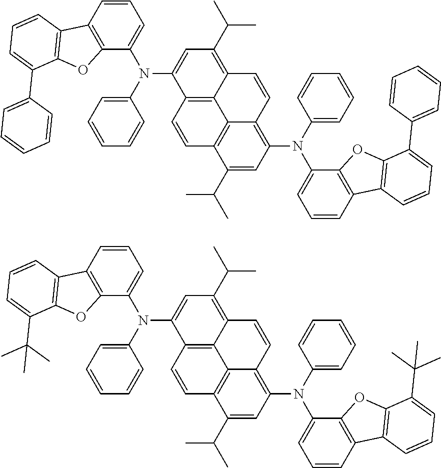

Compound Represented by Formula (4)

The compound represented by the formula (4) will be described.

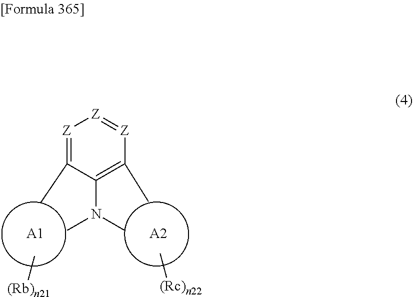

In the formula (4):
Z is each independently CRa or a nitrogen atom;
A1 ring and A2 ring are each independently a substituted or unsubstituted aromatic hydrocarbon ring having 6 to 50 ring carbon atoms, or a substituted or unsubstituted heterocycle having 5 to 50 ring atoms;
when a plurality of Ra are present, at least one combination of adjacent two or more of the plurality of Ra are mutually bonded to form a substituted or unsubstituted monocyclic ring, mutually bonded to form a substituted or unsubstituted fused ring, or not mutually bonded;
n21 and n22 are each independently 0, 1, 2, 3, or 4;
when a plurality of Rb are present, at least one combination of adjacent two or more of the plurality of Rb are mutually bonded to form a substituted or unsubstituted monocyclic ring, mutually bonded to form a substituted or unsubstituted fused ring, or not mutually bonded;
when a plurality of Rc are present, at least one combination of adjacent two or more of the plurality of Rc are mutually bonded to form a substituted or unsubstituted monocyclic ring, mutually bonded to form a substituted or unsubstituted fused ring, or not mutually bonded; and
Ra, Rb and Rc forming neither the substituted or unsubstituted monocyclic ring nor the substituted or unsubstituted fused ring are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

The “aromatic hydrocarbon ring” for the A1 ring and A2 ring has the same structure as a compound formed by introducing a hydrogen atom to the “aryl group” described above.

Ring atoms of the “aromatic hydrocarbon ring” for the A1 ring and the A2 ring include two carbon atoms on a fused bicyclic structure at the center of the formula (4).

Specific examples of the “substituted or unsubstituted aromatic hydrocarbon ring having 6 to 50 ring carbon atoms” include a compound formed by introducing a hydrogen atom to the “aryl group” described in the specific example group G1.

The “heterocycle” for the A1 ring and A2 ring has the same structure as a compound formed by introducing a hydrogen atom to the “heterocyclic group” described above.

Ring atoms of the “heterocycle” for the A1 ring and the A2 ring include two carbon atoms on a fused bicyclic structure at the center of the formula (4).

Specific examples of the “substituted or unsubstituted heterocycle having 5 to 50 ring atoms” include a compound formed by introducing a hydrogen atom to the “heterocyclic group” described in the specific example group G2.

Rb is bonded to any one of carbon atoms forming the aromatic hydrocarbon ring for the A1 ring or any one of the atoms forming the heterocycle for the A1 ring.

Rc is bonded to any one of carbon atoms forming the aromatic hydrocarbon ring for the A2 ring or any one of the atoms forming the heterocycle for the A2 ring.

At least one of Ra, Rb, or Rc is preferably a group represented by a formula (4a) below. More preferably, at least two of Ra, Rb, and Rc are groups represented by the formula (4a).

[Formula 366]

*-L₄₀₁-Ar₄₀₁ (4)

In the formula (4a):
L₄₀₁ is a single bond, a substituted or unsubstituted arylene group having 6 to 30 ring carbon atoms, or a substituted or unsubstituted divalent heterocyclic group having 5 to 30 ring atoms; and
Ar₄₀₁ is a substituted or unsubstituted aryl group having 6 to 50 ring carbon atoms, a substituted or unsubstituted heterocyclic group having 5 to 50 ring atoms, or a group represented by the formula (4b).

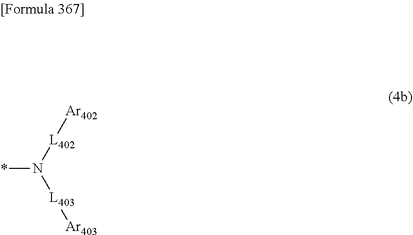

In the formula (4b):
L₄₀₂ and L₄₀₃ are each independently a single bond, a substituted or unsubstituted arylene group having 6 to 30 ring carbon atoms, or a substituted or unsubstituted divalent heterocyclic group having 5 to 30 ring atoms;
a combination of Ar₄₀₂ and Ar₄₀₃ are mutually bonded to form a substituted or unsubstituted monocyclic ring, mutually bonded to form a substituted or unsubstituted fused ring, or not mutually bonded; and
Ar₄₀₂ and Ar₄₀₃ forming neither the substituted or unsubstituted monocyclic ring nor the substituted or unsubstituted fused ring are each independently a substituted or unsubstituted aryl group having 6 to 50 ring carbon atoms, or a substituted or unsubstituted heterocyclic group having 5 to 50 ring atoms.

In an exemplary embodiment, the compound represented by the formula (4) is represented by a formula (42) below.

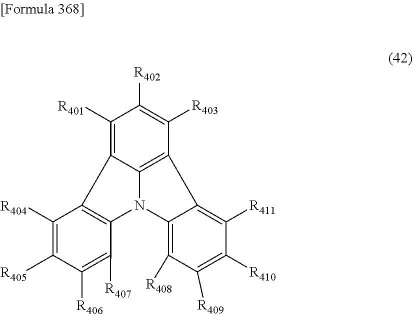

In the formula (42):
at least one combination of adjacent two or more of R₄₀₁ to R₄₁₁ are mutually bonded to form a substituted or unsubstituted monocyclic ring, mutually bonded to form a substituted or unsubstituted fused ring, or not mutually bonded; and
R₄₀₁ to R₄₁₁ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

At least one of R₄₀₁ to R₄₁₁ is preferably a group represented by the formula (4a). More preferably, at least two of R₄₀₁ to R₄₁₁ are each a group represented by the formula (4a).

Preferably, R₄₀₄ and R₄₁₁ are each a group represented by the formula (4a).

In an exemplary embodiment, the compound represented by the formula (4) is a compound formed by bonding a structure represented by a formula (4-1) or a formula (4-2) below to the A1 ring.

Further, in an exemplary embodiment, the compound represented by the formula (42) is a compound formed by bonding a structure represented by the formula (4-1) or the formula (4-2) to the ring bonded with R₄₀₄ to R₄₀₇.

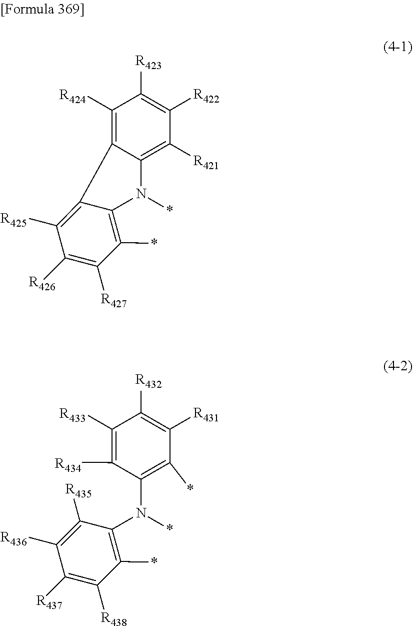

In the formula (4-1), two bonds * are each independently bonded to a ring-forming carbon atom of the aromatic hydrocarbon ring or a ring atom of the heterocycle for the A1 ring in the formula (4) or bonded to one of R₄₀₄ to R₄₀₇ in the formula (42);
in the formula (4-2), three bonds * are each independently bonded to a ring-forming carbon atom of the aromatic hydrocarbon ring or a ring atom of the heterocycle for the A1 ring in the formula (4) or bonded to one of R₄₀₄ to R₄₀₇ in the formula (42);
at least one combination of adjacent two or more of R₄₂₁ to R₄₂₇ are mutually bonded to form a substituted or unsubstituted monocyclic ring, mutually bonded to form a substituted or unsubstituted fused ring, or not mutually bonded;
at least one combination of adjacent two or more of R₄₃₁ to R₄₃₈ are mutually bonded to form a substituted or unsubstituted monocyclic ring, mutually bonded to form a substituted or unsubstituted fused ring, or not mutually bonded; and
R₄₂₁ to R₄₂₇ and R₄₃₁ to R₄₃₈ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In an exemplary embodiment, the compound represented by the formula (4) is a compound represented by a formula (41-3), a formula (41-4), or a formula (41-5) below.

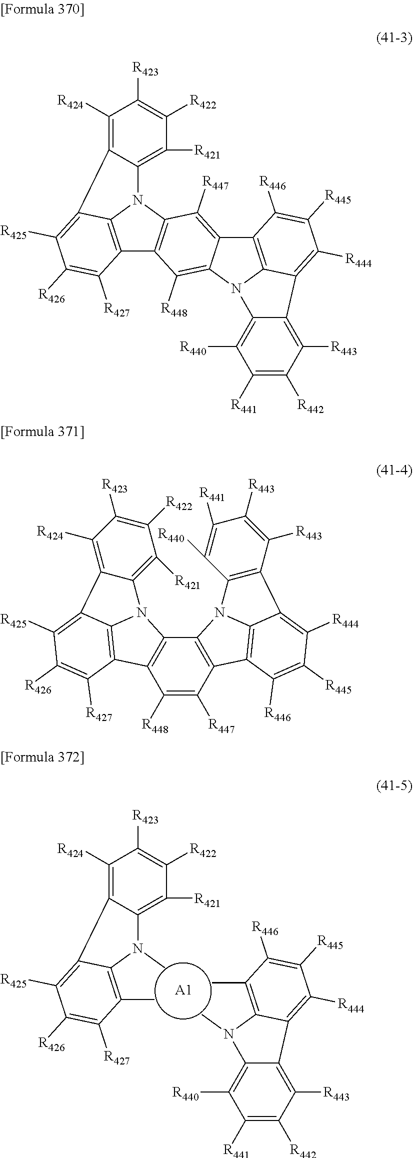

In the formulae (41-3), (41-4) and (41-5):
A1 ring is as defined for the formula (4);
R₄₂₁ to R₄₂₇ each independently represent the same as R₄₂₁ to R₄₂₇ in the formula (4-1); and
R₄₄₀ to R₄₄₈ each independently represent the same as R₄₀₁ to R₄₁₁ in the formula (42).

In an exemplary embodiment, a substituted or unsubstituted aromatic hydrocarbon ring having 6 to 50 ring carbon atoms for the A1 ring in the formula (41-5) is a substituted or unsubstituted naphthalene ring, or a substituted or unsubstituted fluorene ring.

In an exemplary embodiment, a substituted or unsubstituted heterocycle having 5 to 50 ring atoms for the A1 ring in the formula (41-5) is a substituted or unsubstituted dibenzofuran ring, a substituted or unsubstituted carbazole ring, or a substituted or unsubstituted dibenzothiophene ring.

In an exemplary embodiment, the compound represented by the formula (4) or the formula (42) is selected from the group consisting of compounds represented by formulae (461) to (467) below.

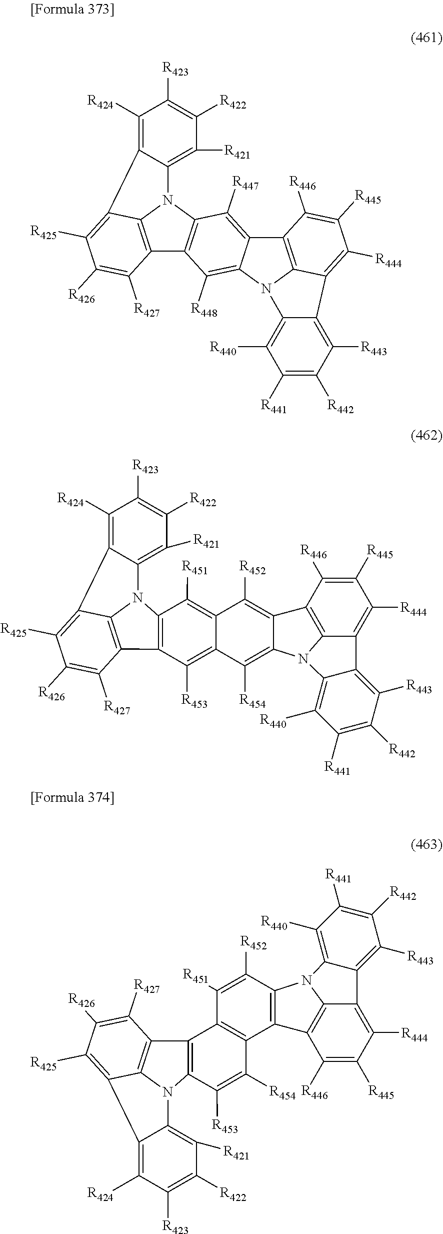

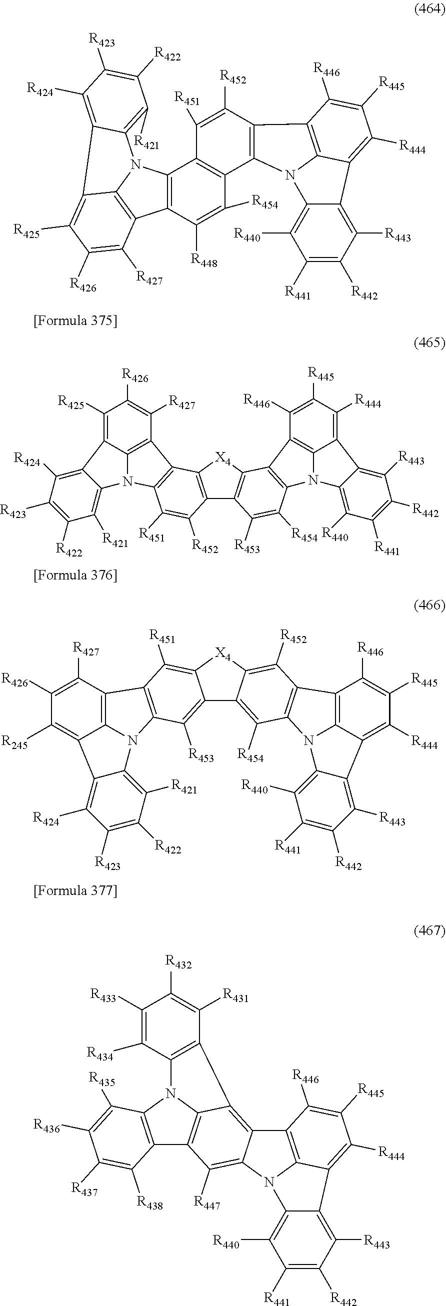

In the formulae (461), (462), (463), (464), (465), (466) and (467):
R₄₂₁ to R₄₂₇ each independently represent the same as R₄₂₁ to R₄₂₇ in the formula (4-1);
R₄₃₁ to R₄₃₈ each independently represent the same as R₄₃₁ to R₄₃₈ in the formula (4-2);
R₄₄₀ to R₄₄₈ and R₄₅₁ to R₄₅₄ each independently represent the same as R₄₀₁ to R₄₁₁ in the formula (42);
X₄ is an oxygen atom, NR₈₀₁, or C(R₈₀₂)(R₈₀₃);
R₈₀₁, R₈₀₂, and R₈₀₃ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
a substituted or unsubstituted alkyl group having 1 to 50 carbon atoms, or a substituted or unsubstituted aryl group having 6 to 50 ring carbon atoms;
when a plurality of R₈₀₁ are present, the plurality of R₈₀₁ are mutually the same or different;
when a plurality of R₈₀₂ are present, the plurality of R₈₀₂ are mutually the same or different; and
when a plurality of R₈₀₃ are present, the plurality of R₈₀₃ are mutually the same or different.

In an exemplary embodiment, in a compound represented by the formula (42), at least one combination of adjacent two or more of R₄₀₁ to R₄₁₁ are mutually bonded to form a substituted or unsubstituted monocyclic ring or a substituted or unsubstituted fused ring. The compound represented by the formula (42) in the exemplary embodiment is described in detail as a compound represented by a formula (45) below.

Compound Represented by Formula (45)

The compound represented by the formula (45) will be described.

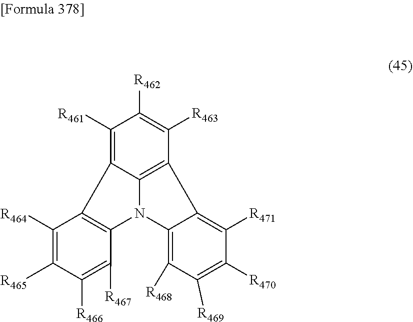

In the formula (45): two or more of combinations selected from the group consisting of a combination of R₄₆₁ and R₄₆₂, a combination of R₄₆₂ and R₄₆₃, a combination of R₄₆₄ and R₄₆₅, a combination of R₄₆₅ and R₄₆₆, a combination of R₄₆₆ and R₄₆₇, a combination of R₄₆₅ and R₄₆₉, a combination of R₄₆₉ and R₄₇₀, and a combination of R₄₇₀ and R₄₇₁ are mutually bonded to form a substituted or unsubstituted monocyclic ring or a substituted or unsubstituted fused ring.

However, the combination of R₄₆₁ and R₄₆₂ and the combination of R₄₆₂ and R₄₆₃; the combination of R₄₆₄ and R₄₆₅ and the combination of R₄₆₅ and R₄₆₆; the combination of R₄₆₅ and R₄₆₆ and the combination of R₄₆₆ and R₄₆₇; the combination of R₄₆₅ and R₄₆₉ and the combination of R₄₆₉ and R₄₇₀; and the combination of R₄₆₉ and R₄₇₀ and the combination of R₄₇₀ and R₄₇₁ do not form a ring at the same time.

At least two rings formed by R₄₆₁ to R₄₇₁ are mutually the same or different.

R₄₆₁ to R₄₇₁ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In the formula (45), Rₙ and Rₙ₊₁ (n being an integer selected from 461, 462, 464 to 466, and 468 to 470) are mutually bonded to form a substituted or unsubstituted monocyclic ring or fused ring together with two ring-forming carbon atoms bonded to Rₙ and Rₙ₊₁. The ring is preferably formed of atoms selected from the group consisting of a carbon atom, an oxygen atom, a sulfur atom, and a nitrogen atom, and is preferably made of 3 to 7, more preferably 5 or 6 atoms.

The number of the above cyclic structures in the compound represented by the formula (45) is, for instance, 2, 3, or 4. The two or more of the cyclic structures may be present on the same benzene ring on the basic skeleton represented by the formula (45) or may be present on different benzene rings. For instance, when three cyclic structures are present, each of the cyclic structures may be present on corresponding one of the three benzene rings of the formula (45).

Examples of the above cyclic structures in the compound represented by the formula (45) include structures represented by formulae (451) to (460) below.

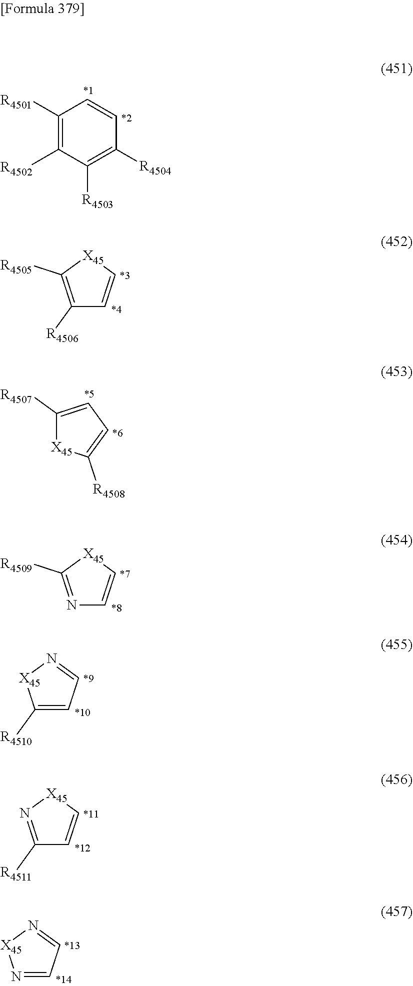

In the formulae (451) to (457):
each combination of *1 and *2, *3 and *4, *5 and *6, *7 and *8, *9 and *10, * 11 and *12, and *13 and *14 represent the two ring-forming carbon atoms bonded to Rₙ and Rₙ₊₁;
the ring-forming carbon atom bonded to Rₙ may be any one of the two ring-forming carbon atoms represented by *1 and *2, *3 and *4, *5 and *6, *7 and *8, *9 and *10, *11 and *12, and *13 and *14;
X₄₅ is C(R₄₅₁₂)(R₄₅₁₃), NR₄₅₁₄, an oxygen atom, or a sulfur atom;
at least one combination of adjacent two or more of R₄₅₀₁ to R₄₅₀₆ and R₄₅₁₂ to R₄₅₁₃ are mutually bonded to form a substituted or unsubstituted monocyclic ring, mutually bonded to form a substituted or unsubstituted fused ring, or not mutually bonded; and
R₄₅₀₁ to R₄₅₁₄ forming neither the substituted or unsubstituted monocyclic ring nor the substituted or unsubstituted fused ring each independently represent the same as R₄₆₁ to R₄₇₁ in the formula (45).

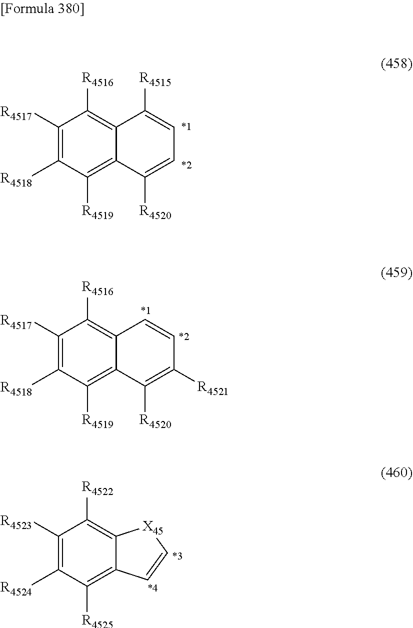

In the formulae (458) to (460):
each combination of *1 and *2, and *3 and *4 represent the two ring-forming carbon atoms bonded to Rₙ and Rₙ₊₁;
the ring-forming carbon atom bonded to Rₙ may be any one of the two ring-forming carbon atoms represented by *1 and *2, or *3 and *4;
X₄₅ is C(R₄₅₁₂)(R₄₅₁₃), NR₄₅₁₄, an oxygen atom, or a sulfur atom;
at least one combination of adjacent two or more of R₄₅₁₂ to R₄₅₁₃ and R₄₅₁₅ to R₄₅₂₅ are mutually bonded to form a substituted or unsubstituted monocyclic ring, mutually bonded to form a substituted or unsubstituted fused ring, or not mutually bonded; and
R₄₅₁₂ to R₄₅₁₃, R₄₅₁₅ to R₄₅₂₁ and R₄₅₂₂ to R₄₅₂₅ forming neither the substituted or unsubstituted monocyclic ring nor the substituted or unsubstituted fused ring, and R₄₅₁₄ each independently represent the same as R₄₆₁ to R₄₇₁ in the formula (45).

In the formula (45), it is preferable that at least one of R₄₆₂, R₄₆₄, R₄₆₅, R₄₇₀ or R₄₇₁ (preferably, at least one of R₄₆₂, R₄₆₅, or R₄₇₀, more preferably R₄₆₂) is a group forming no cyclic structure.
(i) In the formula (45), a substituent, if present, for a cyclic structure formed by Rₙ and Rₙ₊₁,
(ii) in the formula (45), R₄₆₁ to R₄₇₁ forming no cyclic structure, and
(iii) R₄₅₀₁ to R₄₅₁₄ and R₄₅₁₅ to R₄₅₂₅ in the formulae (451) to (460) are preferably each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —N(R₉₀₆)(R₉₀₇), a substituted or unsubstituted aryl group having 6 to 50 ring carbon atoms, a substituted or unsubstituted heterocyclic group having 5 to 50 ring atoms, or groups represented by formulae (461) to (464).

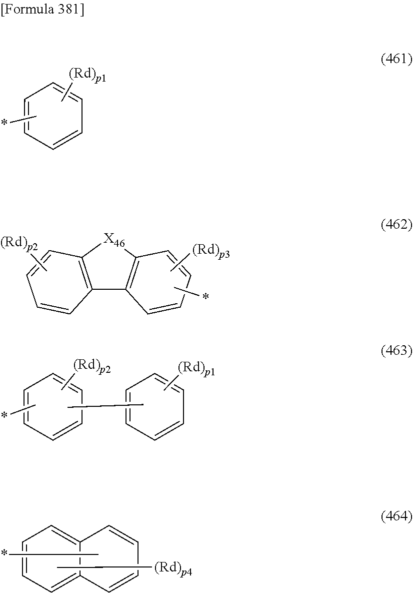

In the formulae (461) to (464):
R_{d} are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
X₄₆ is C(R₈₀₁)(R₈₀₂), NR₈₀₃, an oxygen atom, or a sulfur atom;
R₈₀₁, R₈₀₂, and R₈₀₃ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
a substituted or unsubstituted alkyl group having 1 to 50 carbon atoms, or a substituted or unsubstituted aryl group having 6 to 50 ring carbon atoms;
when a plurality of R₈₀₁ are present, the plurality of R₈₀₁ are mutually the same or different;
when a plurality of R₈₀₂ are present, the plurality of R₈₀₂ are mutually the same or different;
when a plurality of R₈₀₃ are present, the plurality of R₈₀₃ are mutually the same or different;
p1 is 5;
p2 is 4;
p3 is 3;
p4 is 7; and
* in the formulae (461) to (464) each independently represents a bonding position to a cyclic structure.

In the third and fourth compounds, R₉₀₁ to R₉₀₇ represent the same as those as described above.

In an exemplary embodiment, the compound represented by the formula (45) is represented by one of formulae (45-1) to (45-6) below.

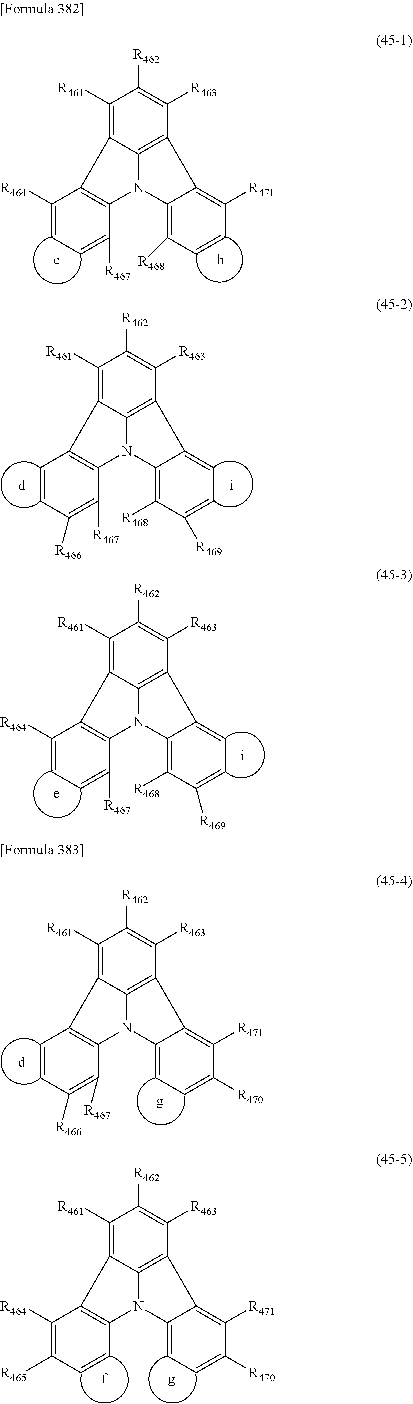

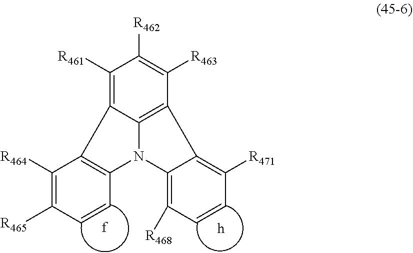

In the formulae (45-1) to (45-6):
rings d to i are each independently a substituted or unsubstituted monocyclic ring or a substituted or unsubstituted fused ring; and
R₄₆₁ to R₄₇₁ each independently represent the same as R₄₆₁ to R₄₇₁ in the formula (45).

In an exemplary embodiment, the compound represented by the formula (45) is represented by one of formulae (45-7) to (45-12) below.

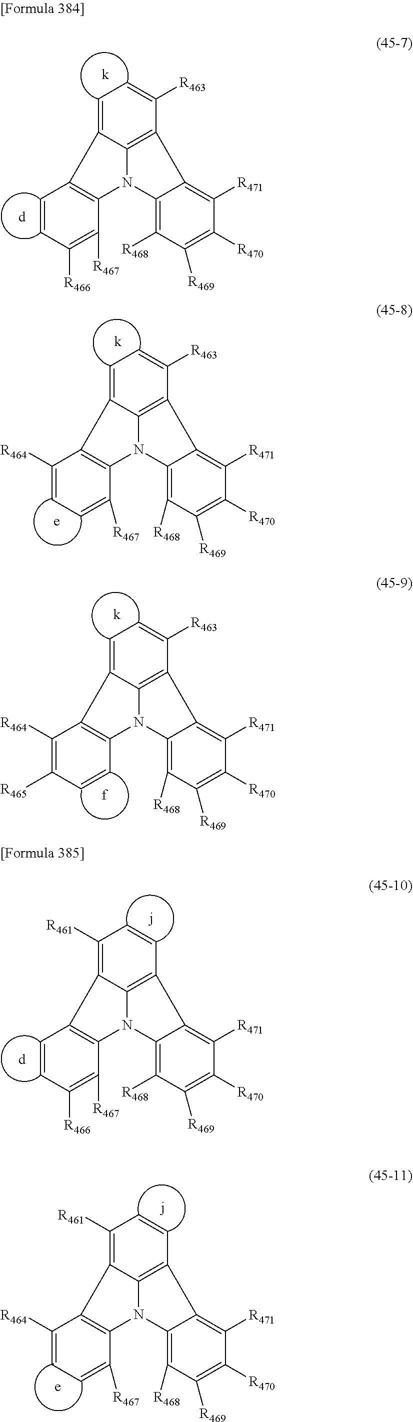

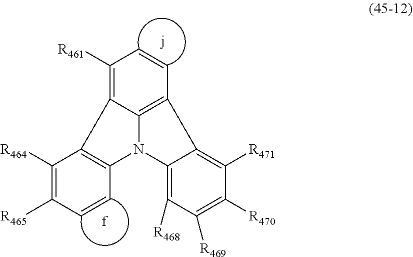

In the formulae (45-7) to (45-12):
rings d to f, k, and j are each independently a substituted or unsubstituted monocyclic ring or a substituted or unsubstituted fused ring; and
R₄₆₁ to R₄₇₁ each independently represent the same as R₄₆₁ to R₄₇₁ in the formula (45).

In an exemplary embodiment, the compound represented by the formula (45) is represented by one of formulae (45-13) to (45-21) below.

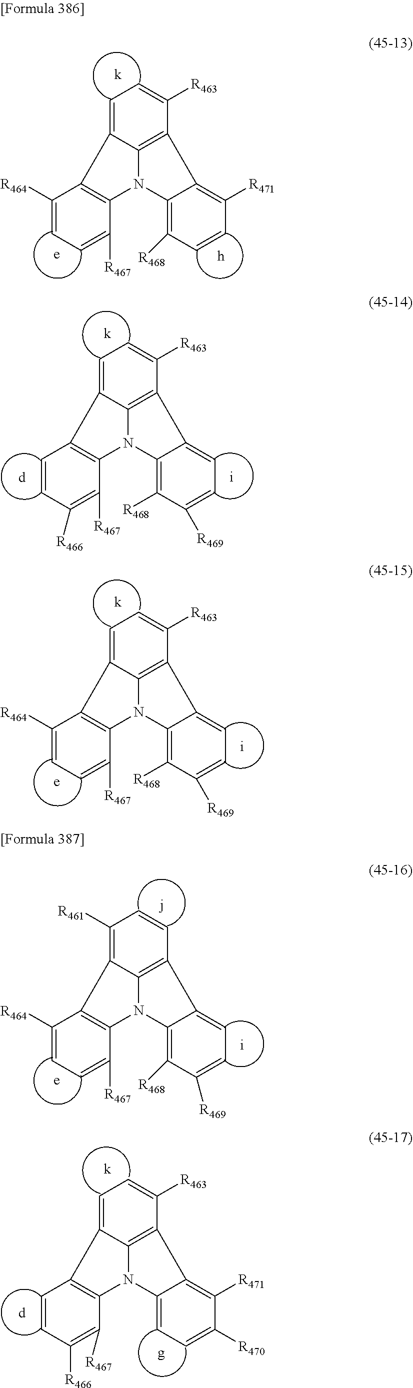

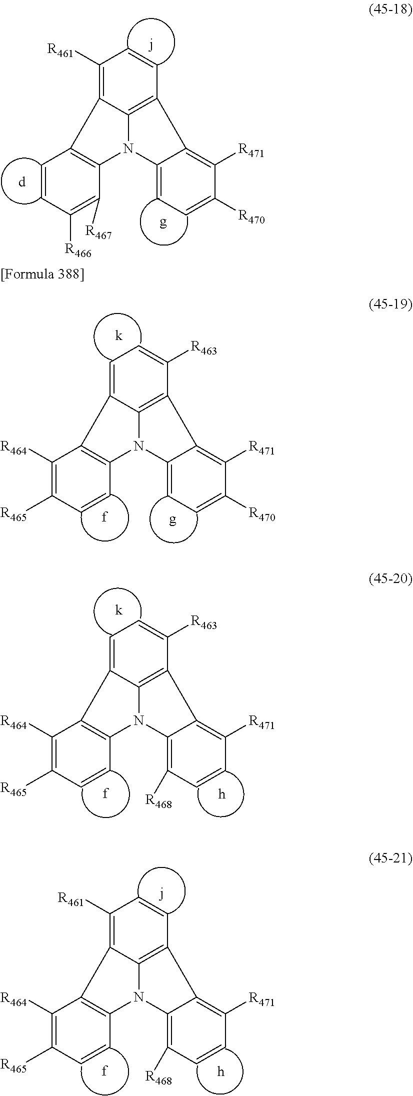

In the formulae (45-13) to (45-21):
rings d to k are each independently a substituted or unsubstituted monocyclic ring or a substituted or unsubstituted fused ring; and
R₄₆₁ to R₄₇₁ each independently represent the same as R₄₆₁ to R₄₇₁ in the formula (45).

When the ring g or the ring h further has a substituent, examples of the substituent include a substituted or unsubstituted alkyl group having 1 to 50 carbon atoms, a substituted or unsubstituted aryl group having 6 to 50 ring carbon atoms, a group represented by the formula (461), a group represented by the formula (463), and a group represented by the formula (464).

In an exemplary embodiment, the compound represented by the formula (45) is represented by one of formulae (45-22) to (45-25) below.

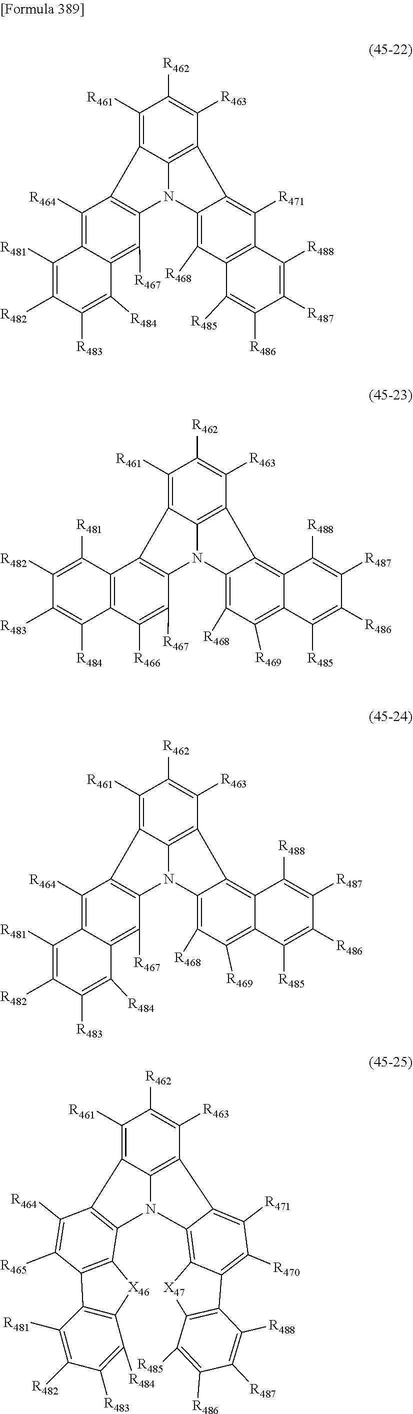

In the formulae (45-22) to (45-25):
X₄₆ and X₄₇ are each independently C(R₈₀₁)(R₈₀₂), NR₈₀₃, an oxygen atom, or a sulfur atom;
R₄₆₁ to R₄₇₁ and R₄₈₁ to R₄₈₈ each independently represent the same as R₄₆₁ to R₄₇₁ of the formula (45);
R₈₀₁, R₈₀₂, and R₈₀₃ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
a substituted or unsubstituted alkyl group having 1 to 50 carbon atoms, or a substituted or unsubstituted aryl group having 6 to 50 ring carbon atoms;
when a plurality of R₈₀₁ are present, the plurality of R₈₀₁ are mutually the same or different;
when a plurality of R₈₀₂ are present, the plurality of R₈₀₂ are mutually the same or different; and
when a plurality of R₈₀₃ are present, the plurality of R₈₀₃ are mutually the same or different.

In an exemplary embodiment, the compound represented by the formula (45) is represented by a formula (45-26) below.

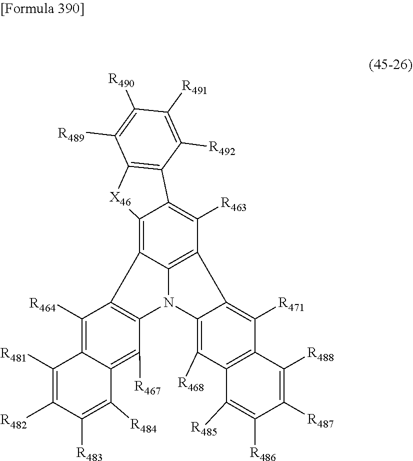

In the formula (45-26):
X₄₆ is C(R₈₀₁)(R₈₀₂), NR₈₀₃, an oxygen atom, or a sulfur atom;
R₄₆₃, R₄₆₄, R₄₆₇, R₄₆₅, R₄₇₁, and R₄₈₁ to R₄₉₂ each independently represent the same as R₄₆₁ to R₄₇₁ in the formula (45);
R₈₀₁, R₈₀₂, and R₈₀₃ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
a substituted or unsubstituted alkyl group having 1 to 50 carbon atoms, or a substituted or unsubstituted aryl group having 6 to 50 ring carbon atoms;
when a plurality of R₈₀₁ are present, the plurality of R₈₀₁ are mutually the same or different;
when a plurality of R₈₀₂ are present, the plurality of R₈₀₂ are mutually the same or different; and
when a plurality of R₈₀₃ are present, the plurality of R₈₀₃ are mutually the same or different.

Specific examples of the compound represented by the formula (4) include compounds shown below. In the specific examples below, Ph represents a phenyl group, and D represents a deuterium atom.

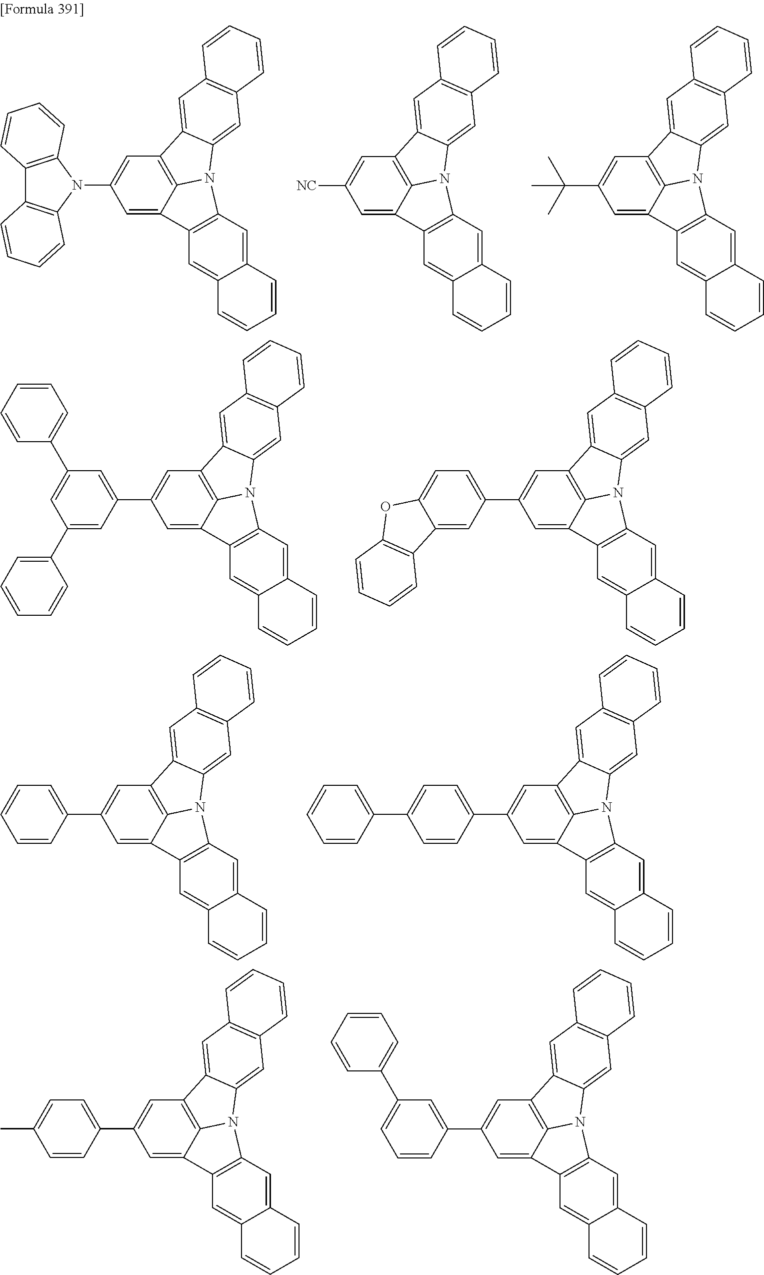

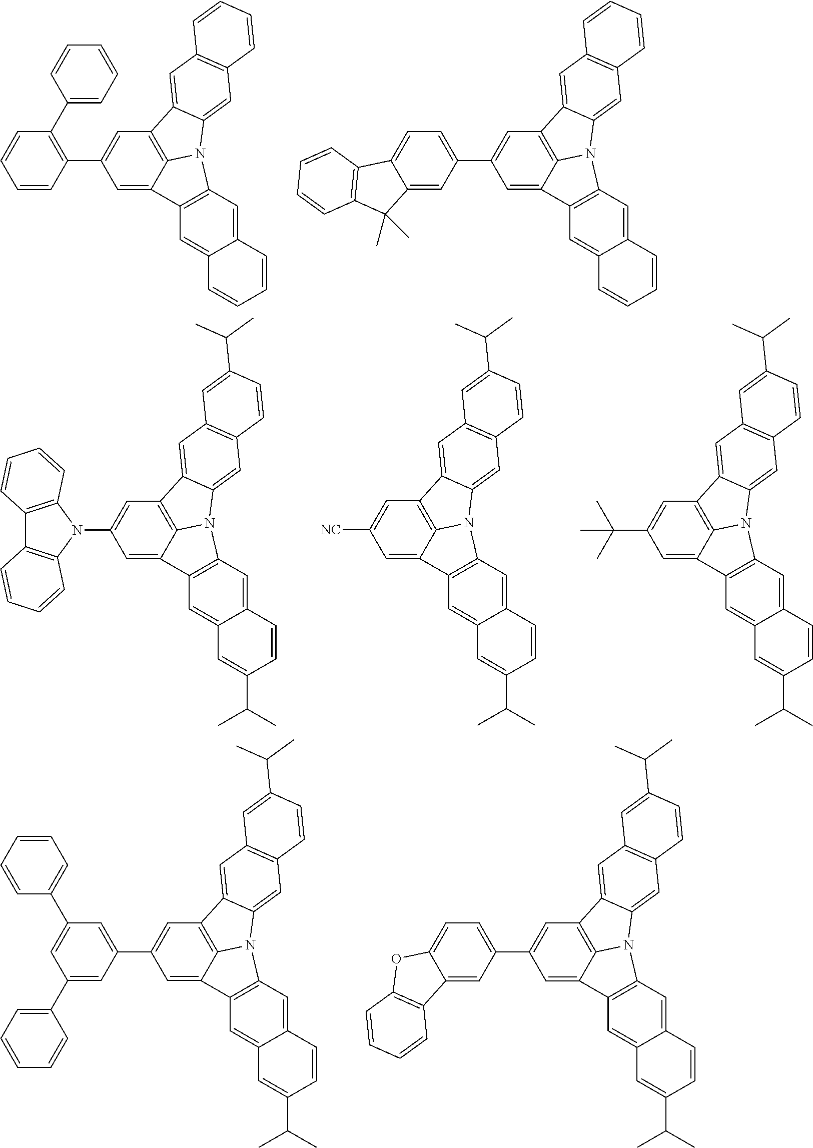

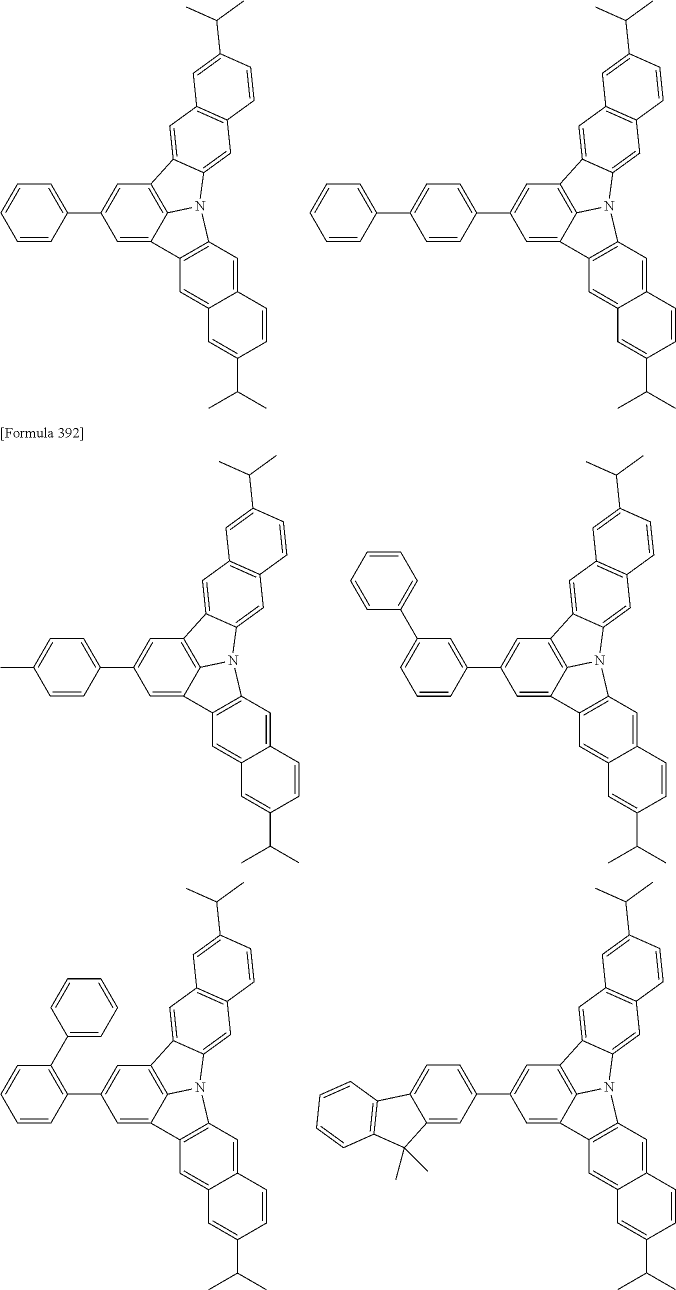

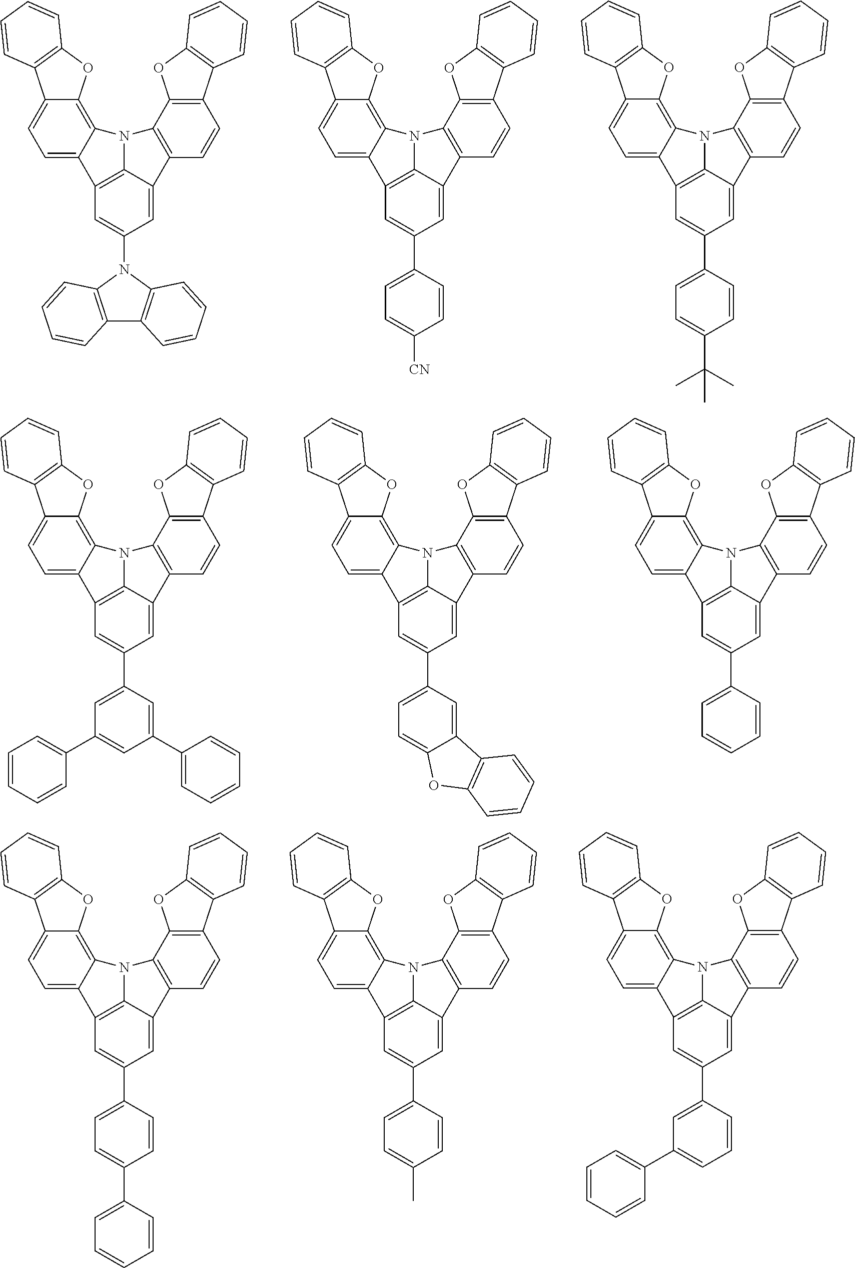

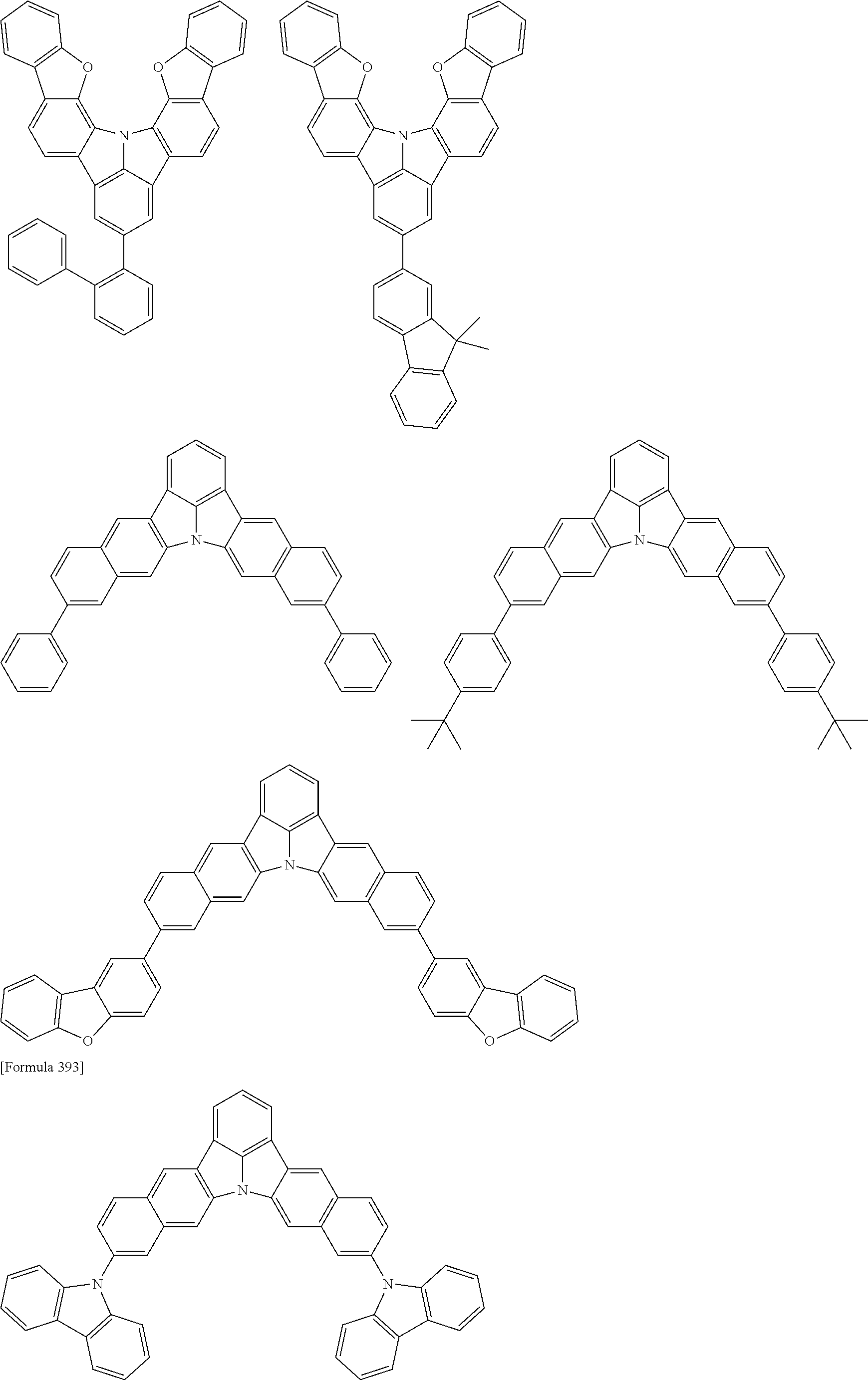

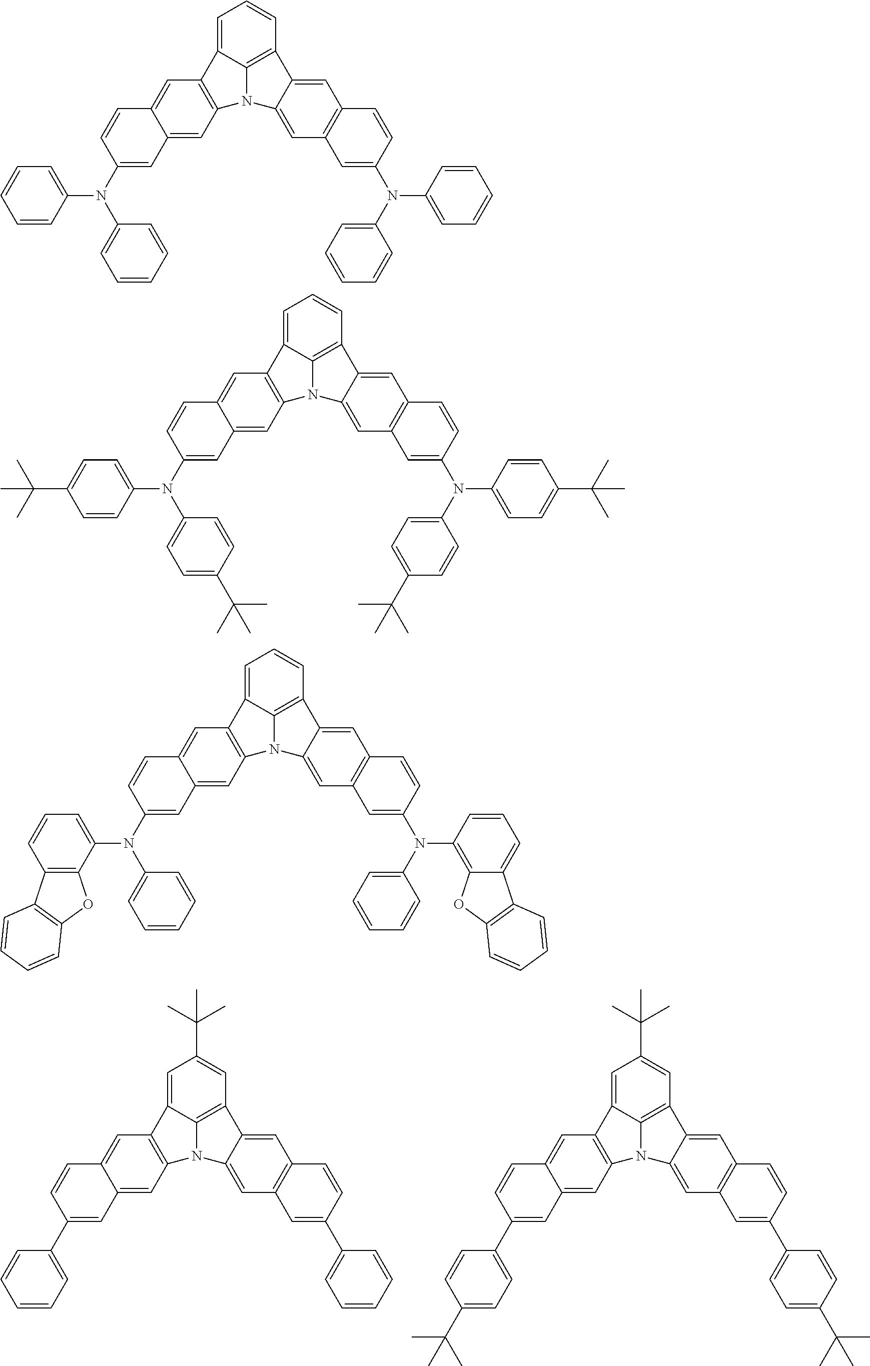

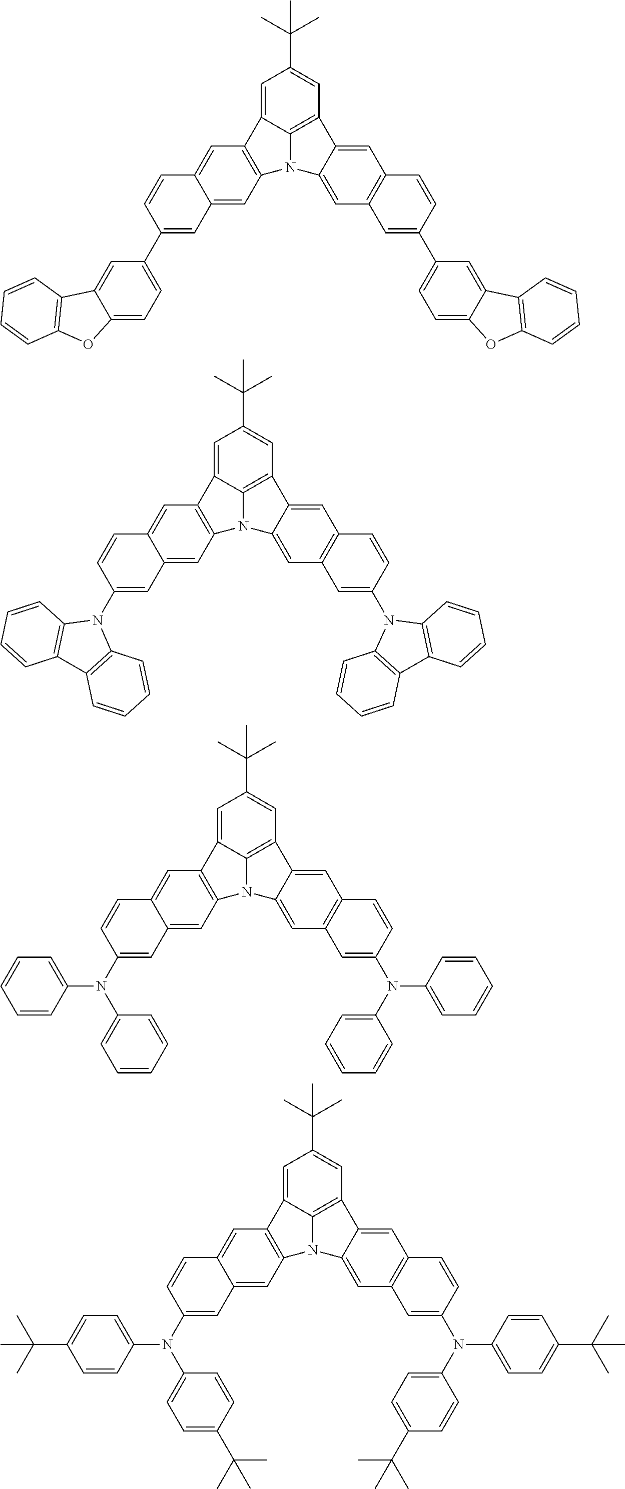

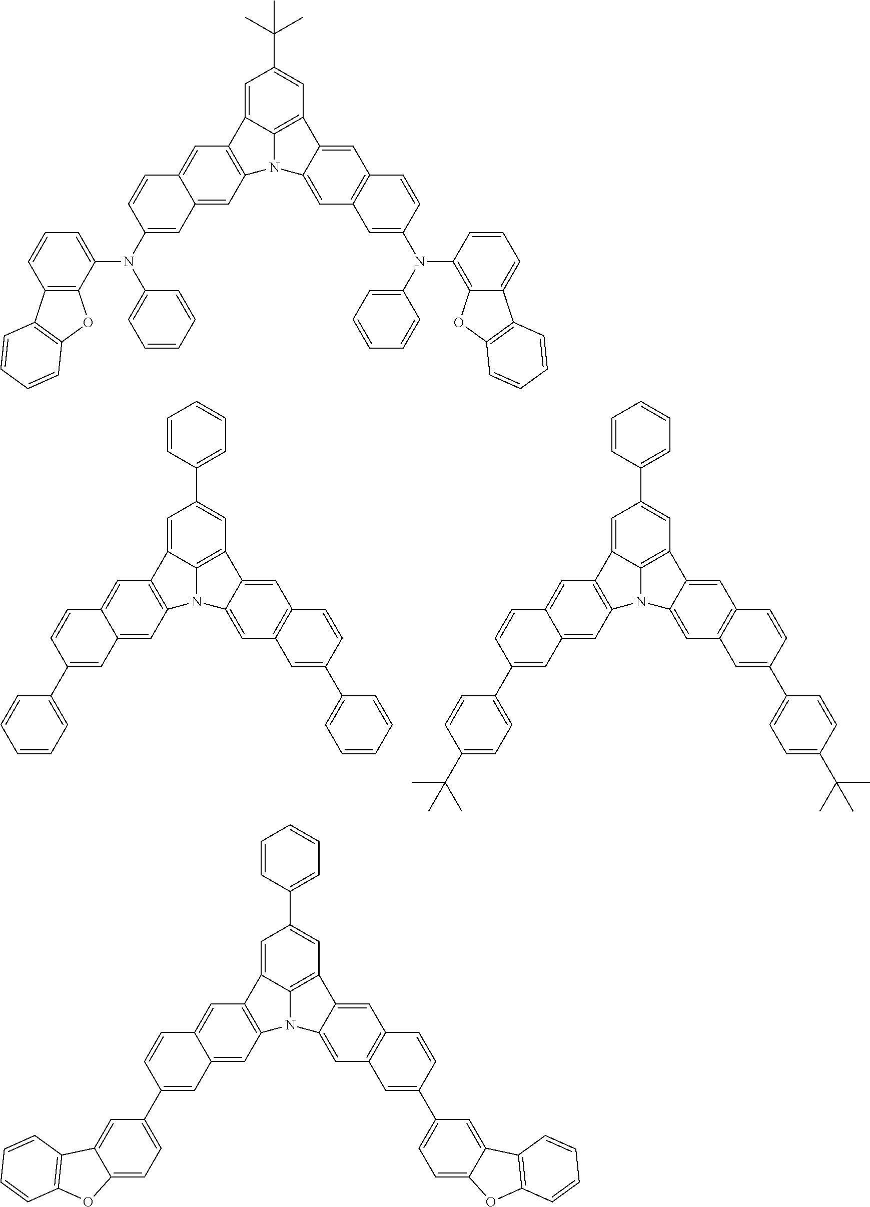

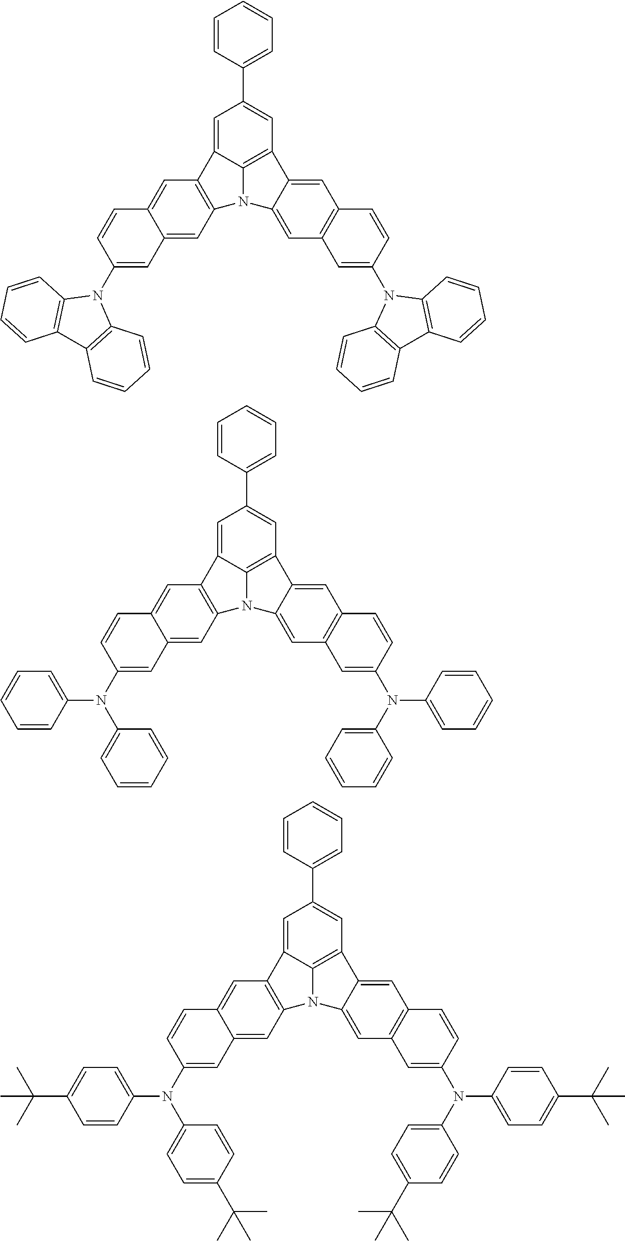

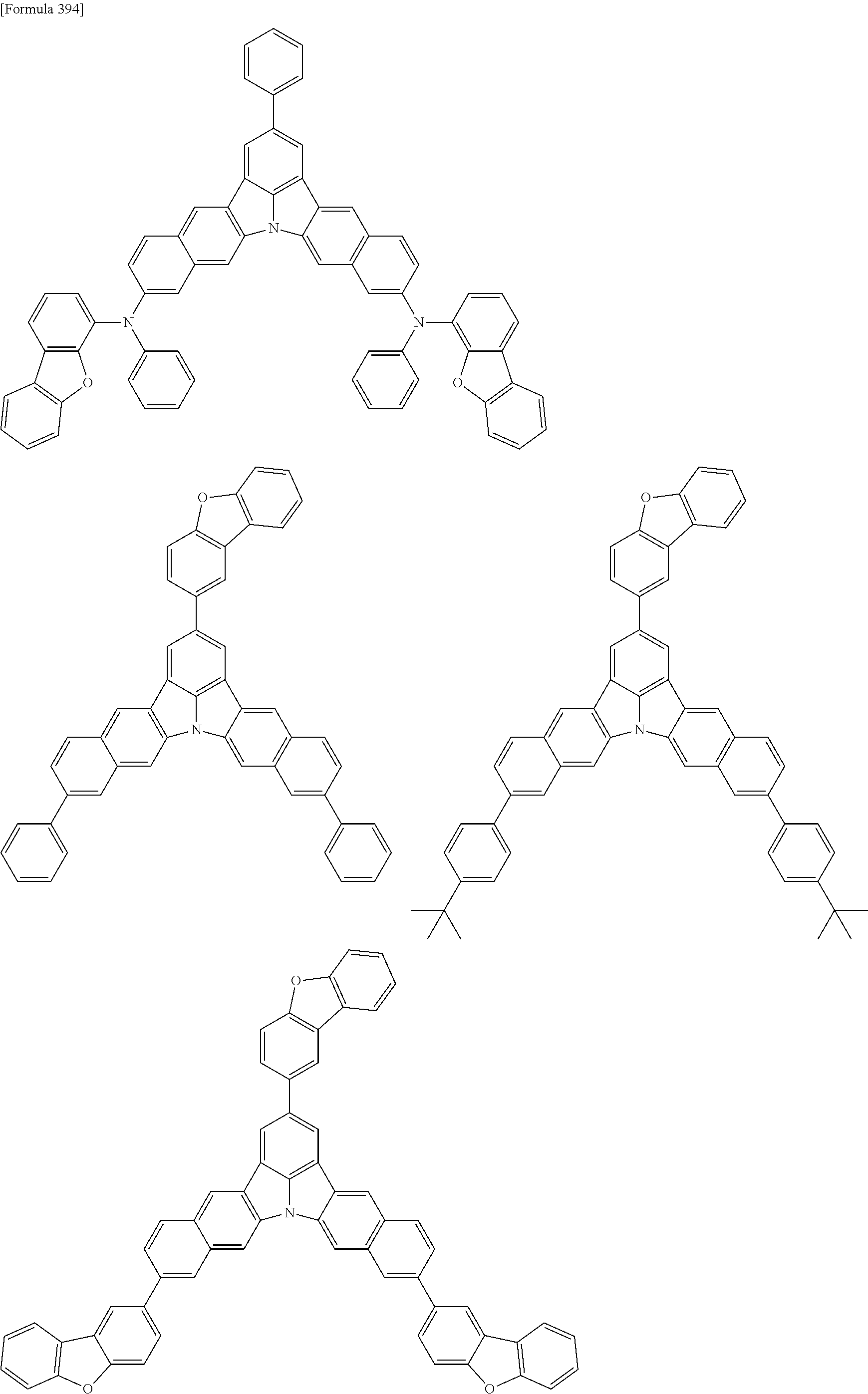

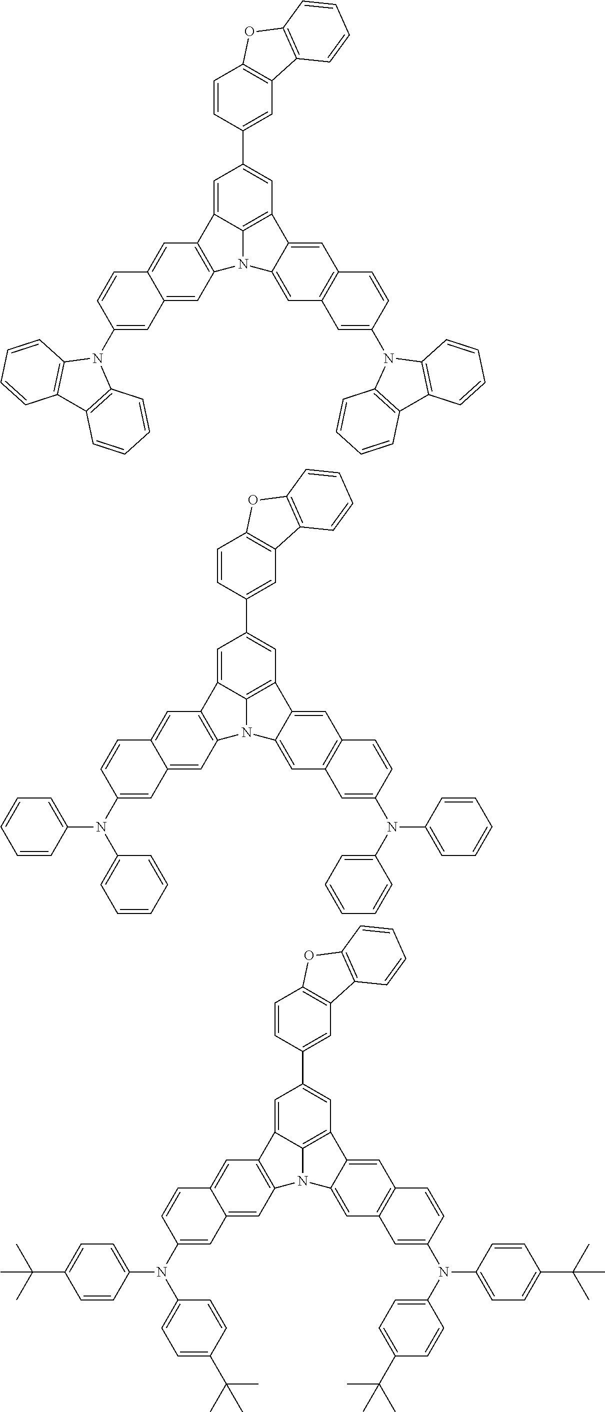

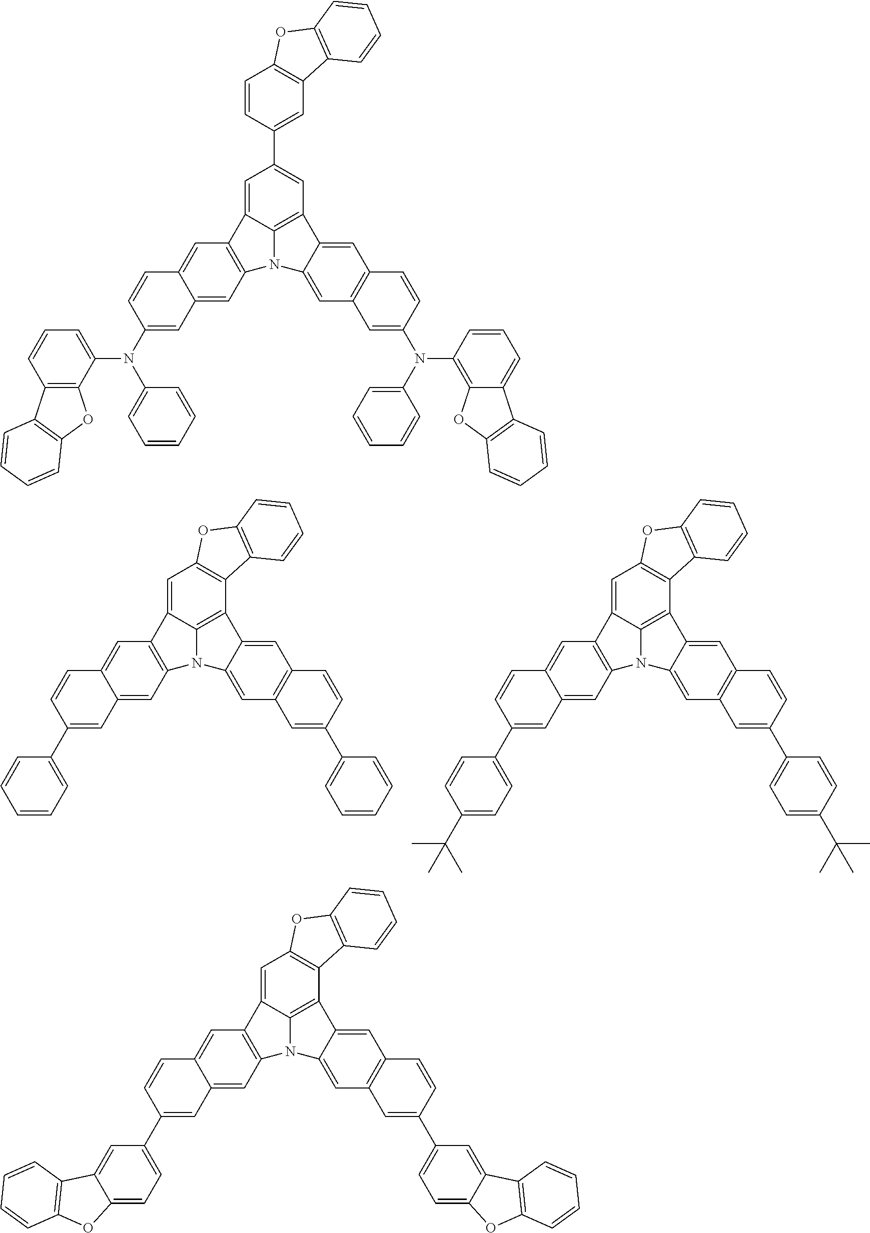

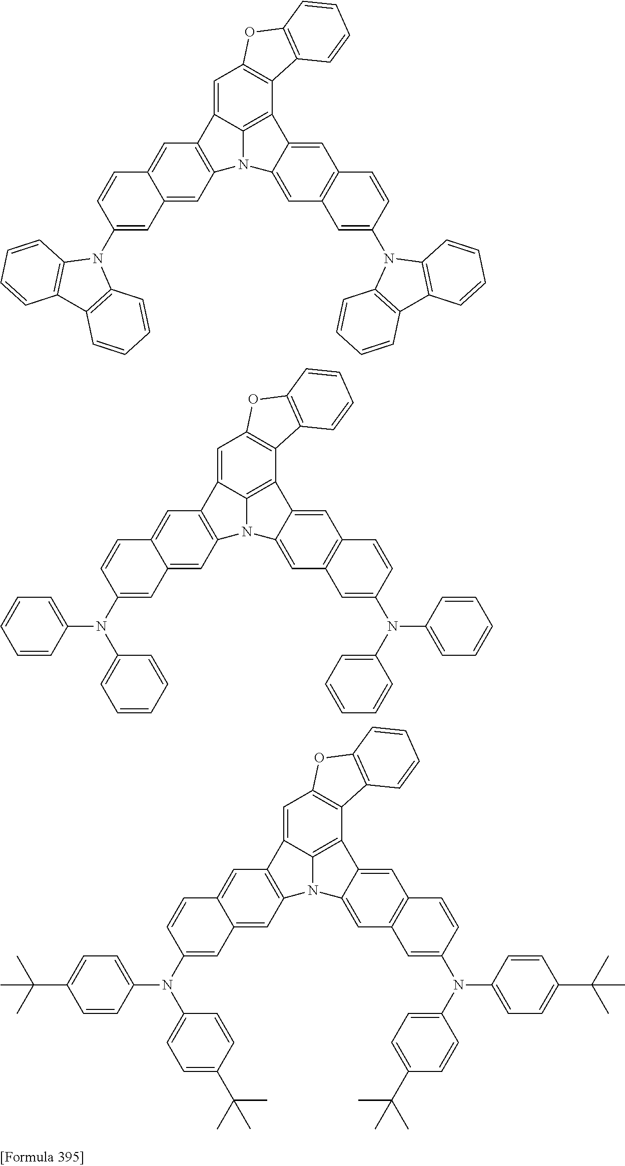

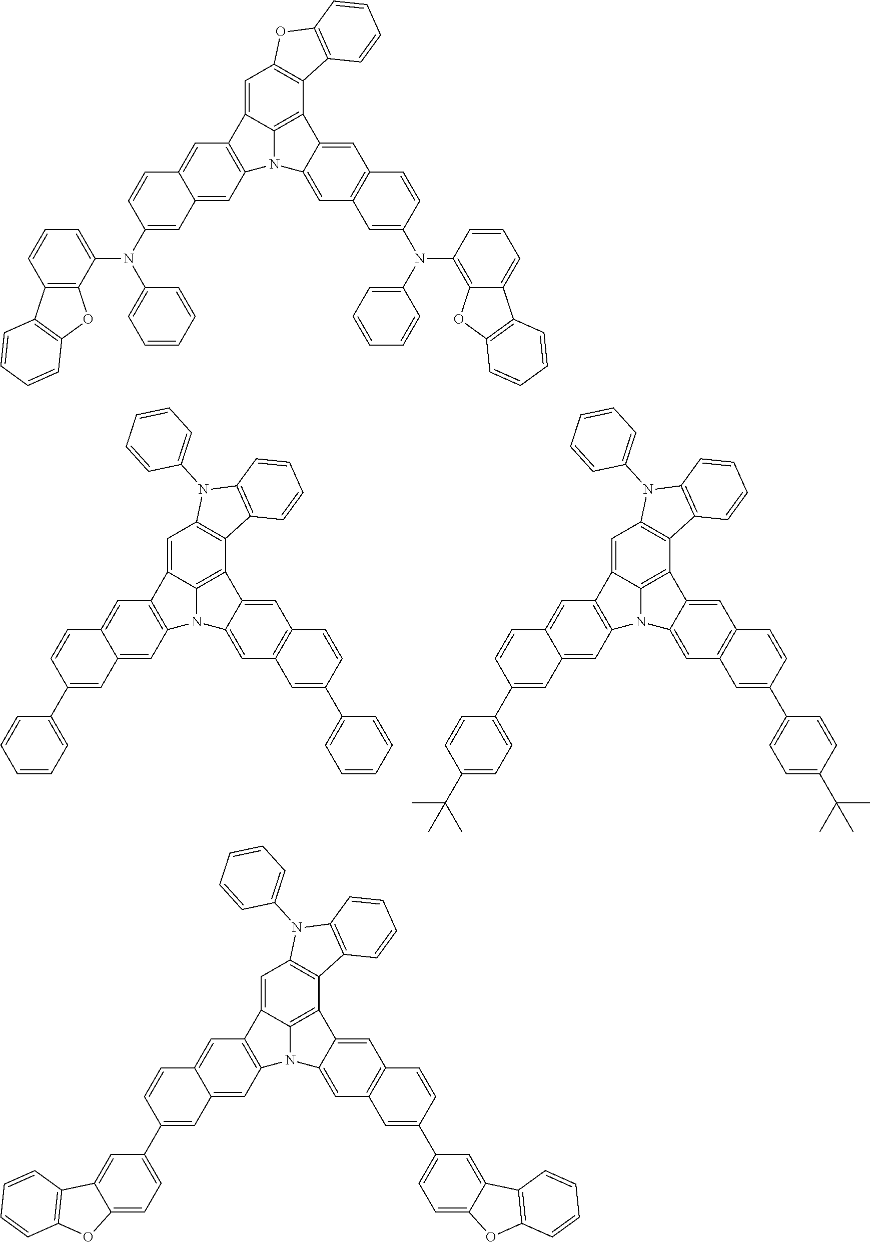

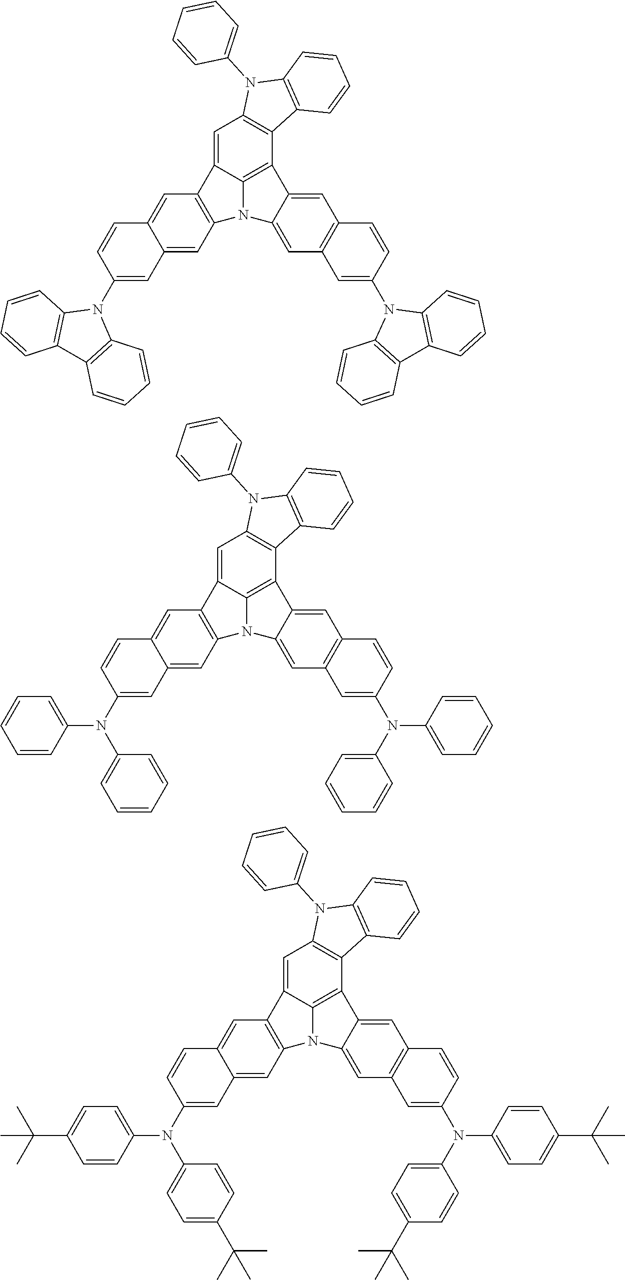 [SMILES: c1ccc(-n2c3ccccc3c3c4c5cc6ccc(-n7c8ccccc8c8ccccc87)cc6cc5n5c6cc7cc(-n8c9ccccc9c9ccccc98)ccc7cc6c(cc32)c45)cc1 ; c1ccc(N(c2ccccc2)c2ccc3cc4c5cc6c(c7ccccc7n6-c6ccccc6)c6c7cc8ccc(N(c9ccccc9)c9ccccc9)cc8cc7n(c4cc3c2)c56)cc1 ; CC(C)(C)c1ccc(N(c2ccc(C(C)(C)C)cc2)c2ccc3cc4c5cc6c(c7ccccc7n6-c6ccccc6)c6c7cc8ccc(N(c9ccc(C(C)(C)C)cc9)c9ccc(C(C)(C)C)cc9)cc8cc7n(c4cc3c2)c56)cc1]

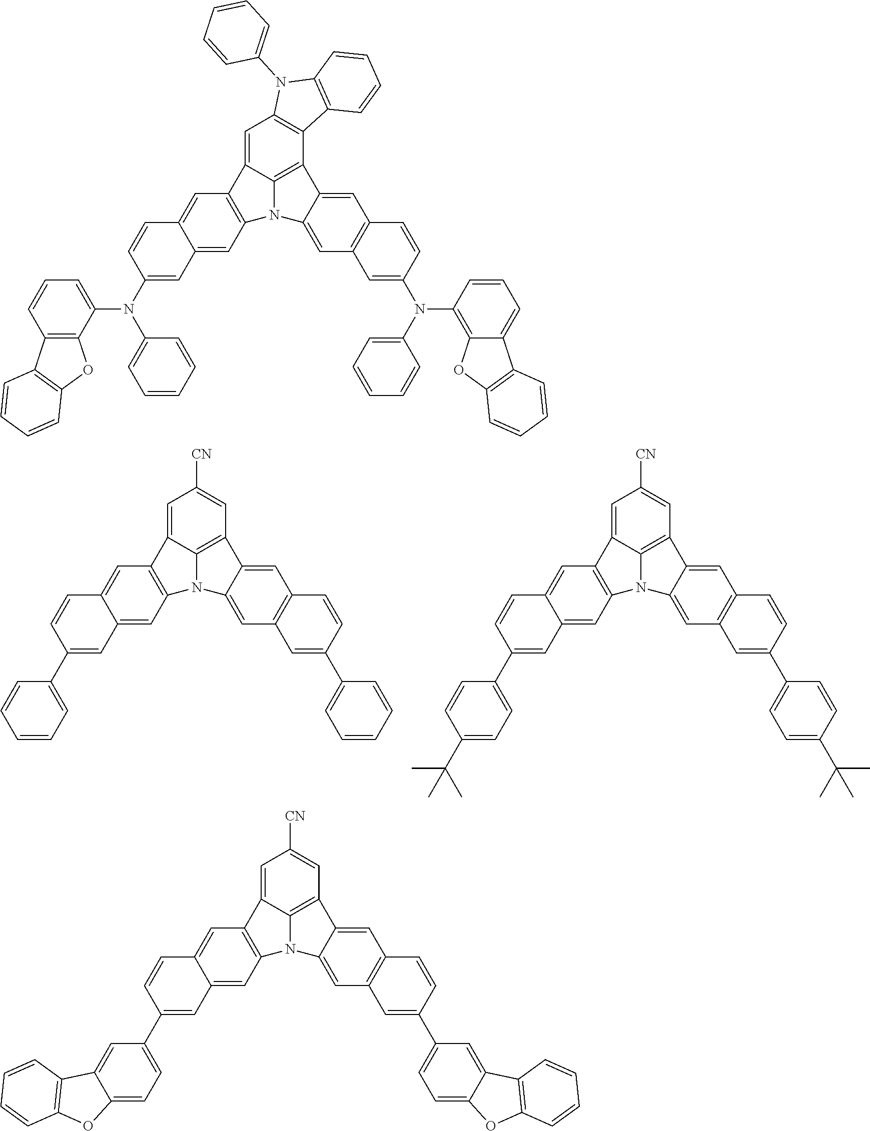

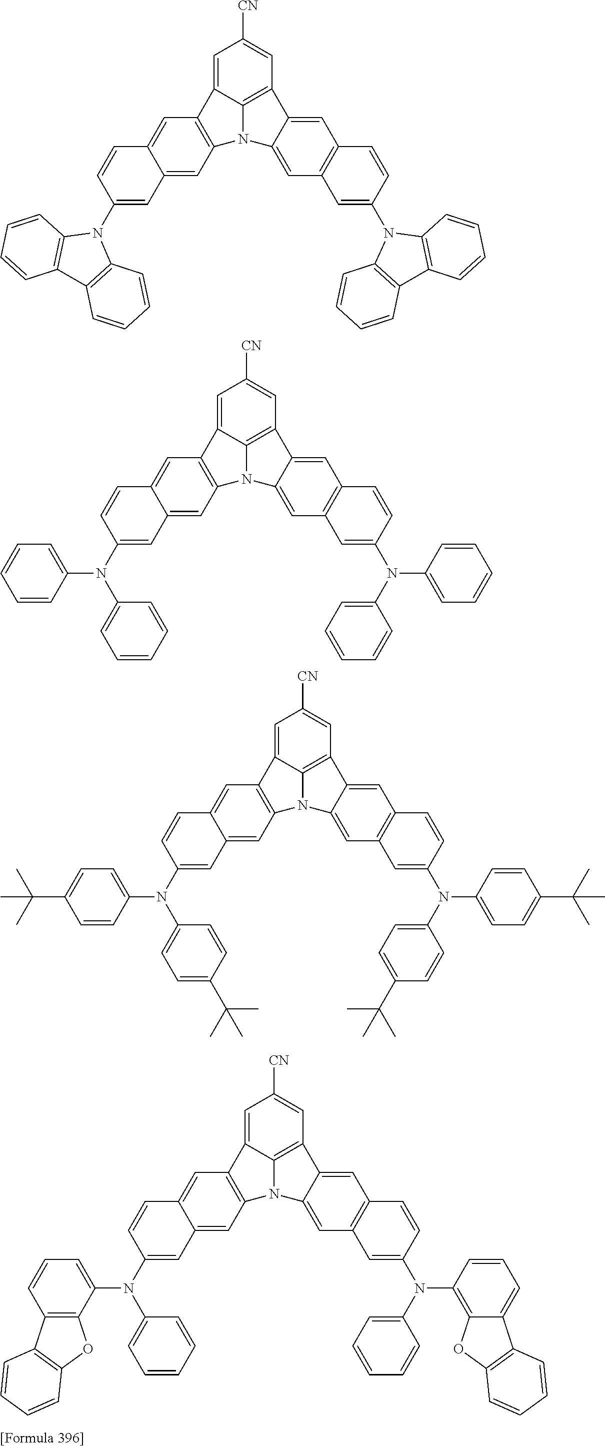

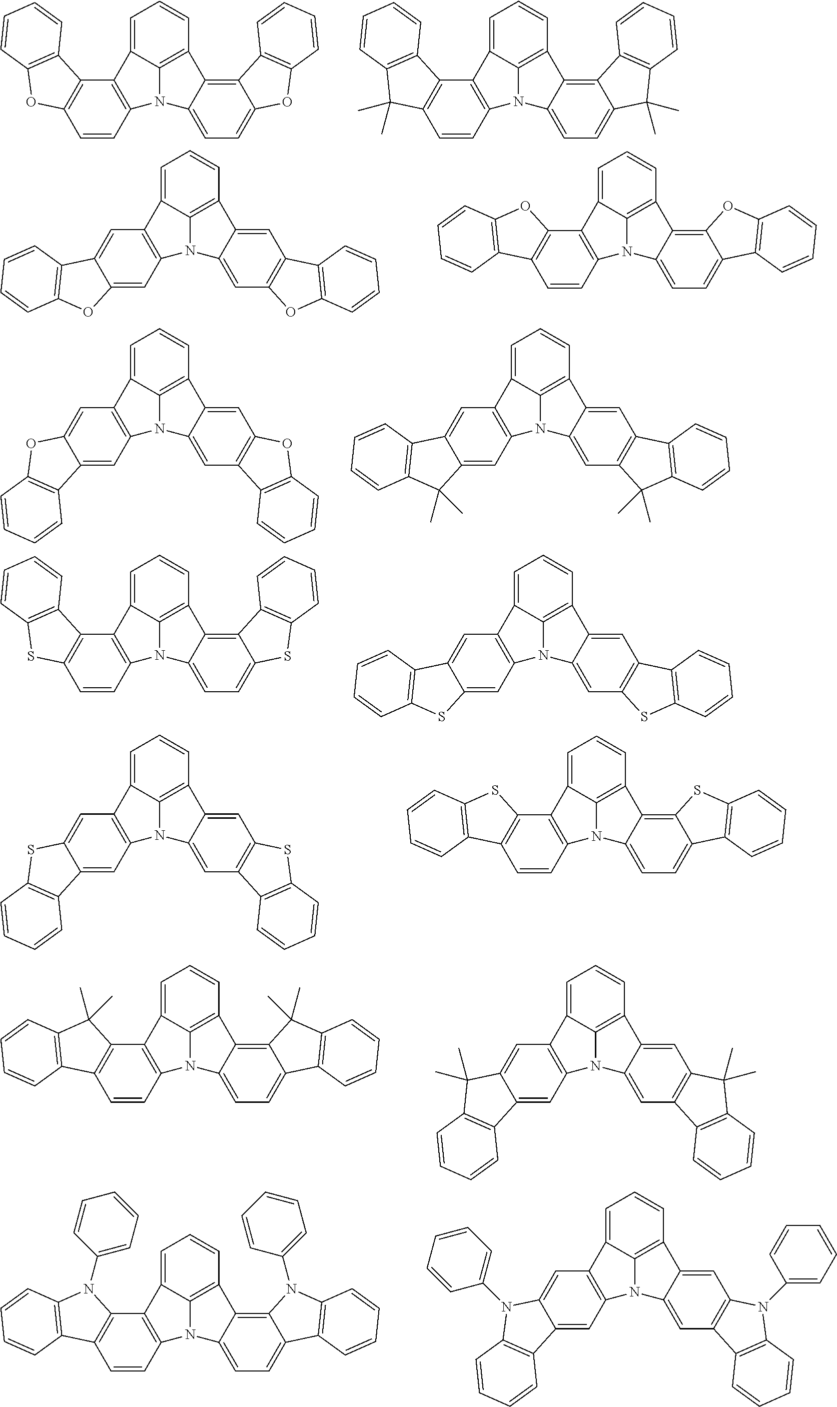

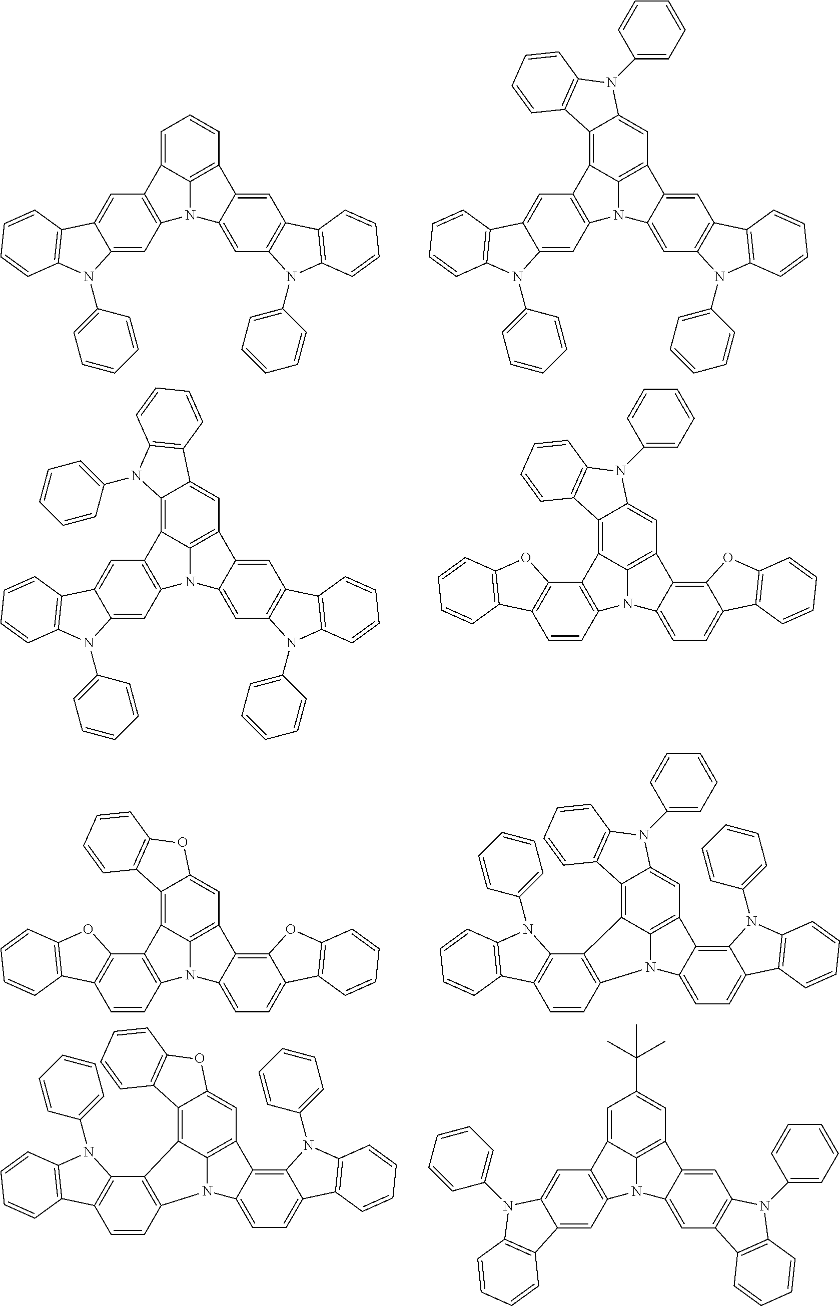

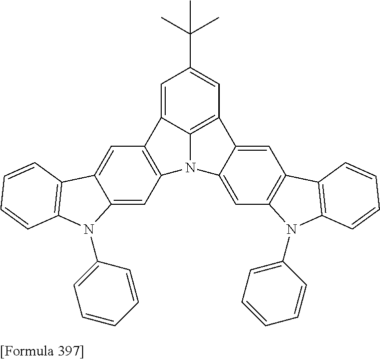 [SMILES: CC(C)(C)c1cc2c3cc4c5ccccc5n(-c5ccccc5)c4cc3n3c4cc5c(cc4c(c1)c23)c1ccccc1n5-c1ccccc1]

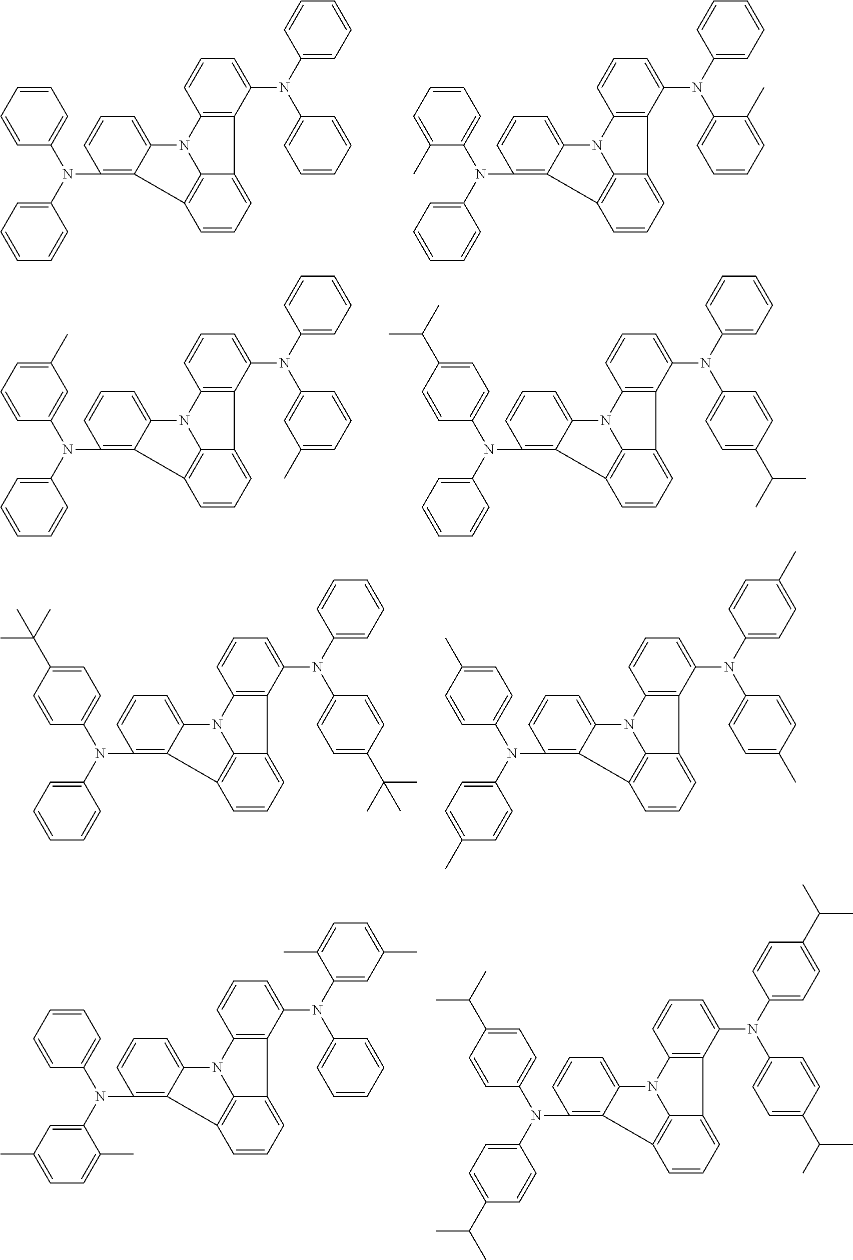

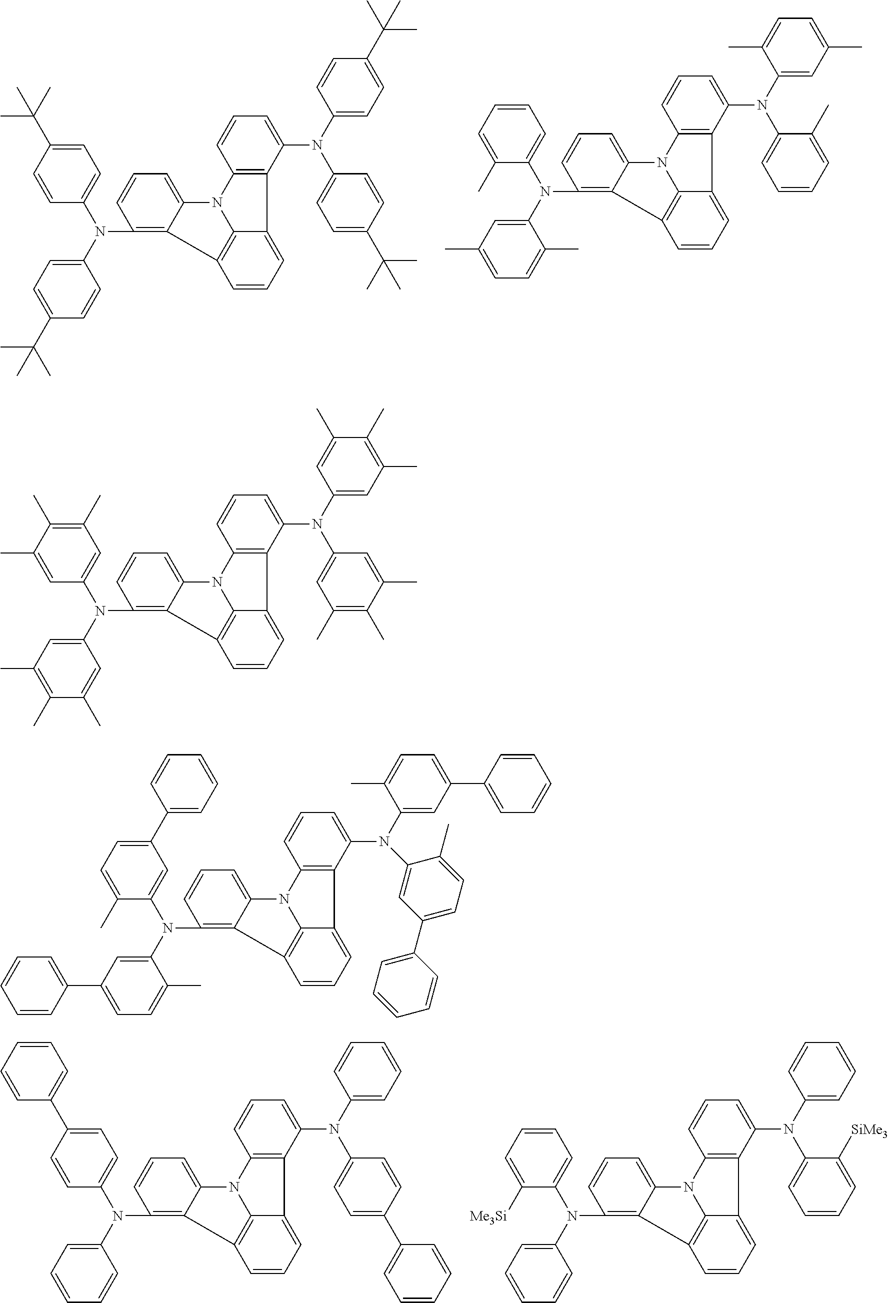

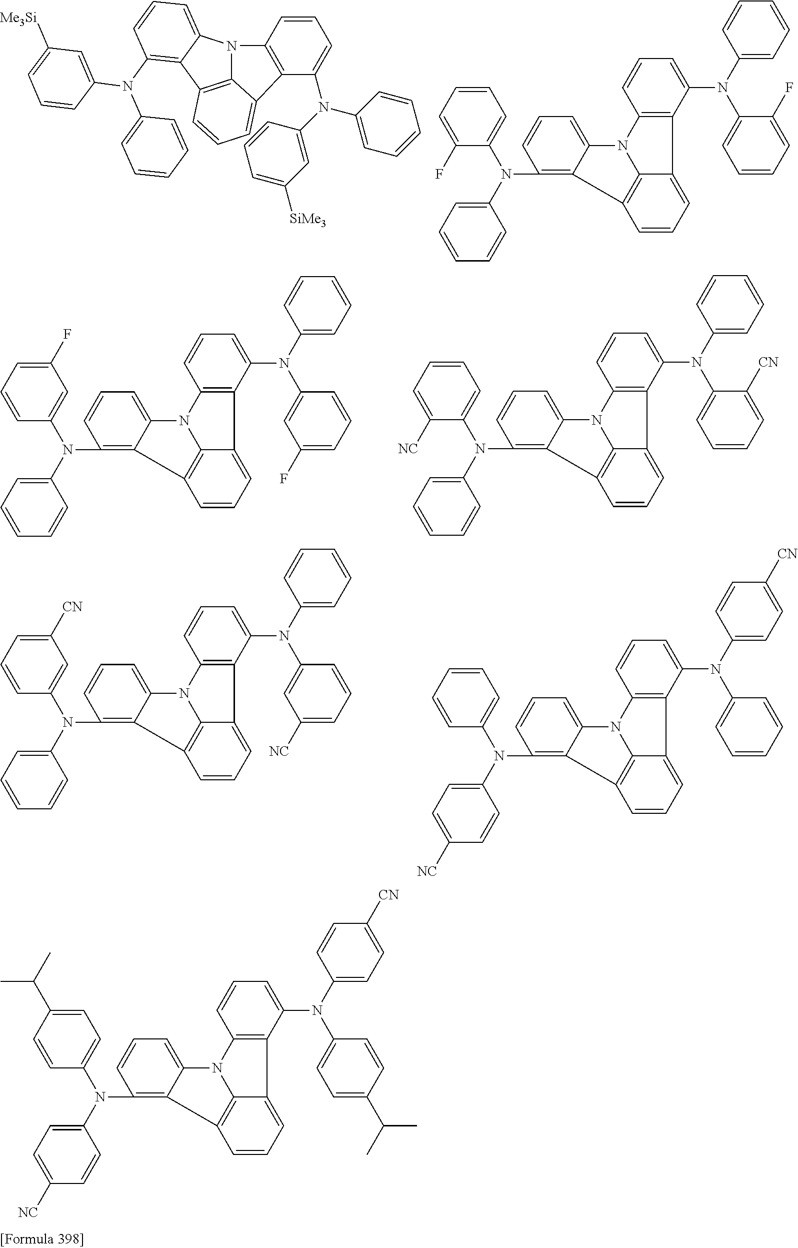

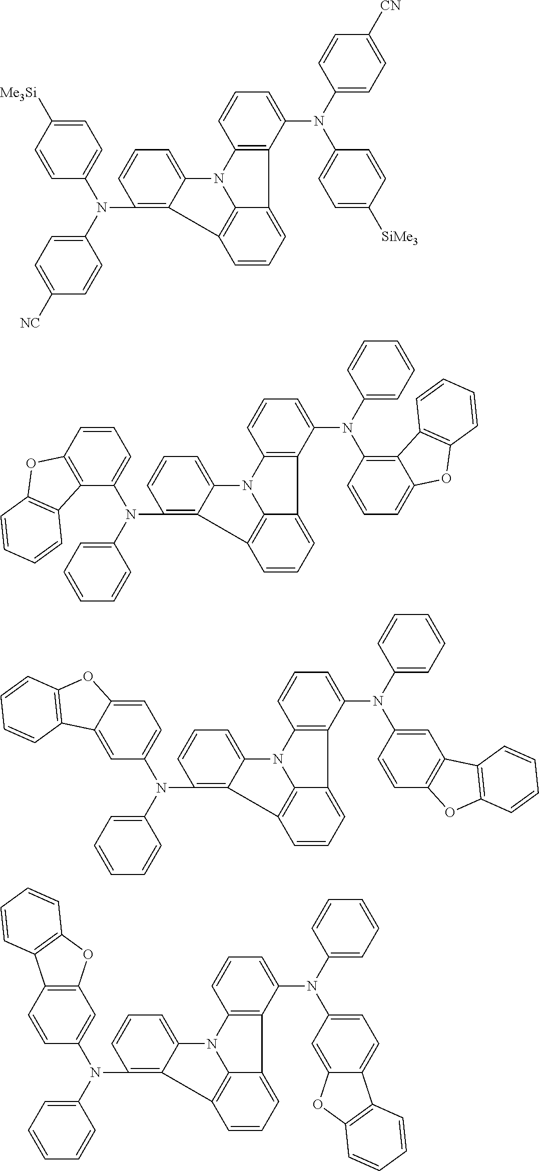 [SMILES: C[Si](C)(C)c1ccc(N(c2ccc(C#N)cc2)c2cccc3c2c2cccc4c5c(N(c6ccc(C#N)cc6)c6ccc([Si](C)(C)C)cc6)cccc5n3c24)cc1 ; c1ccc(N(c2cccc3oc4ccccc4c23)c2cccc3c2c2cccc4c5c(N(c6ccccc6)c6cccc7oc8ccccc8c67)cccc5n3c24)cc1 ; c1ccc(N(c2ccc3oc4ccccc4c3c2)c2cccc3c2c2cccc4c5c(N(c6ccccc6)c6ccc7oc8ccccc8c7c6)cccc5n3c24)cc1 ; c1ccc(N(c2ccc3c(c2)oc2ccccc23)c2cccc3c2c2cccc4c5c(N(c6ccccc6)c6ccc7c(c6)oc6ccccc67)cccc5n3c24)cc1]

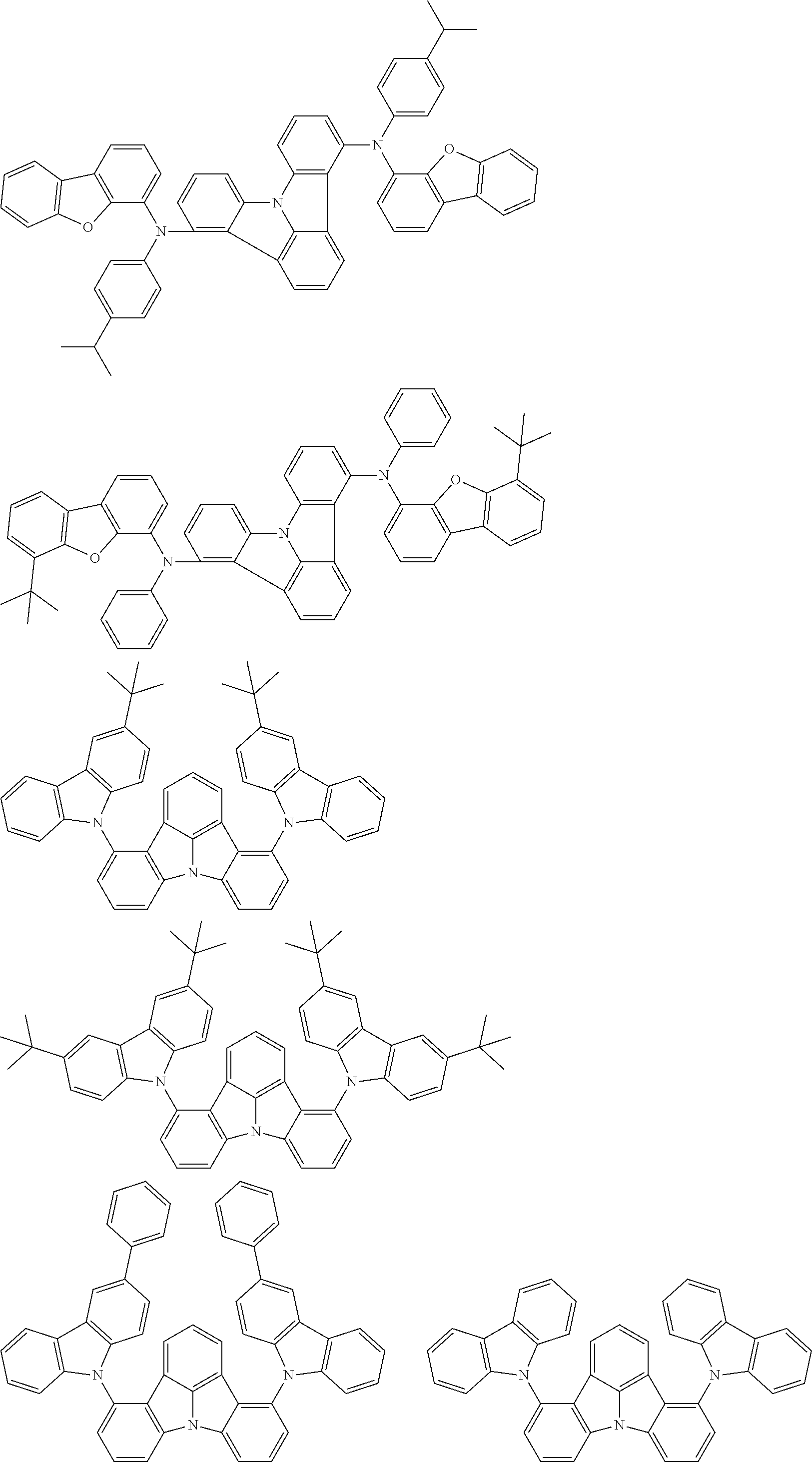

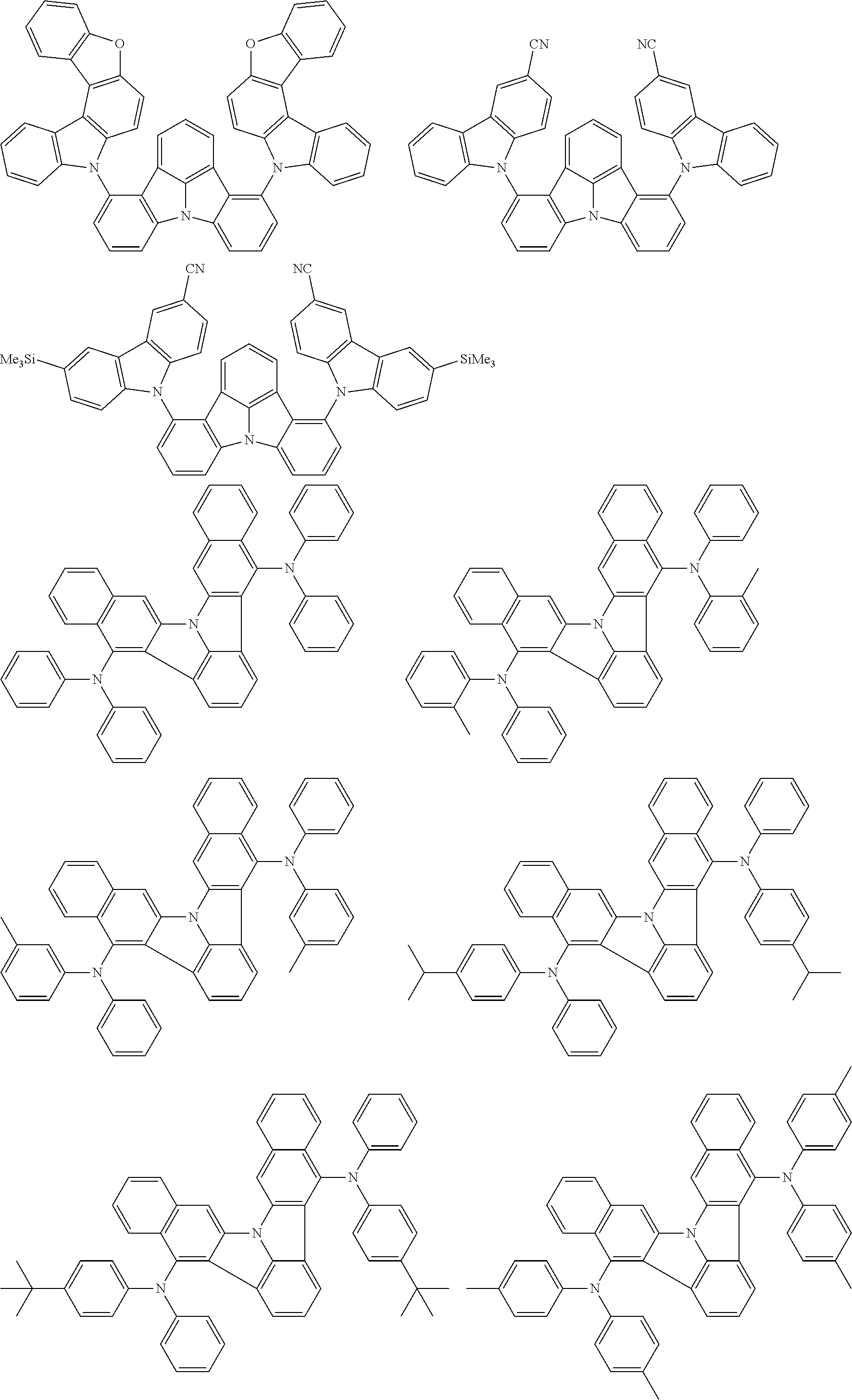

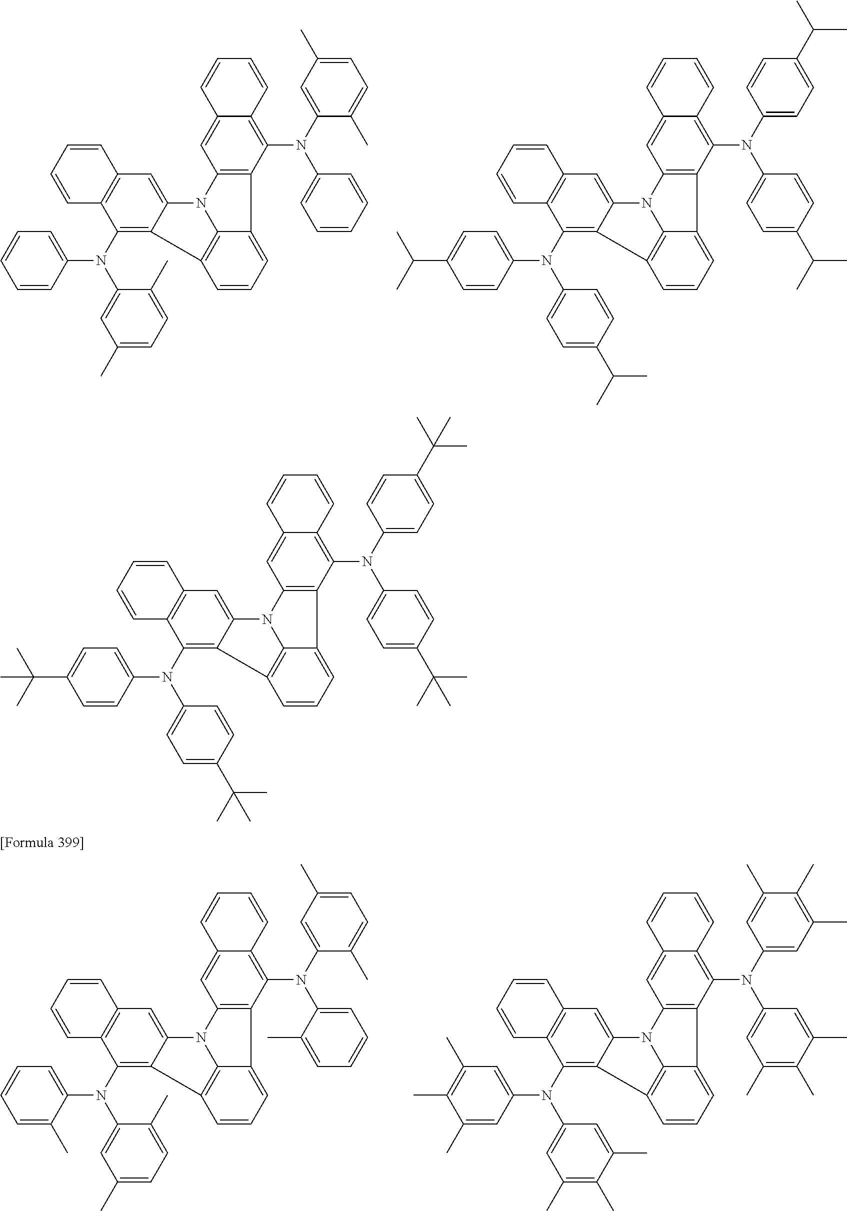

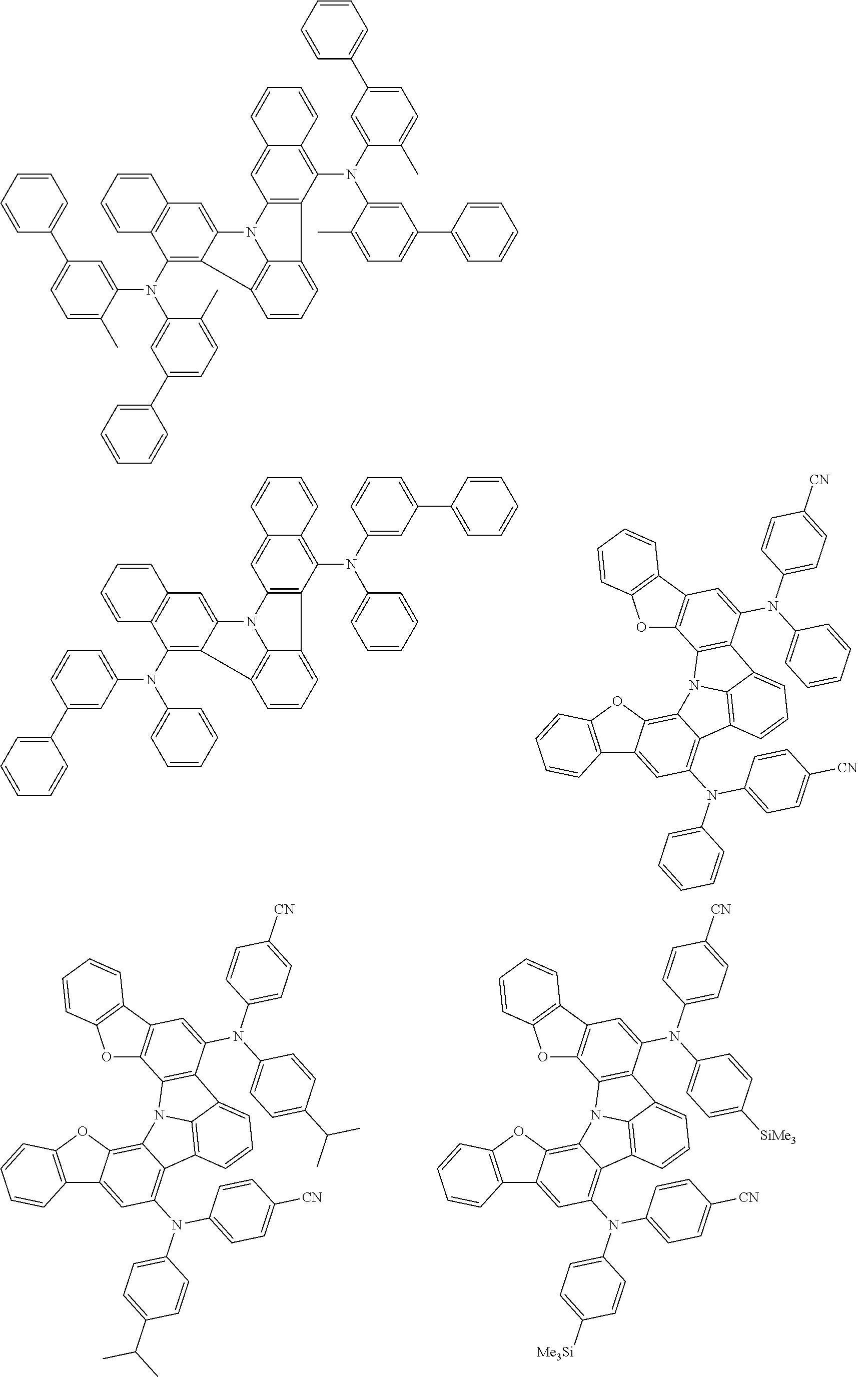

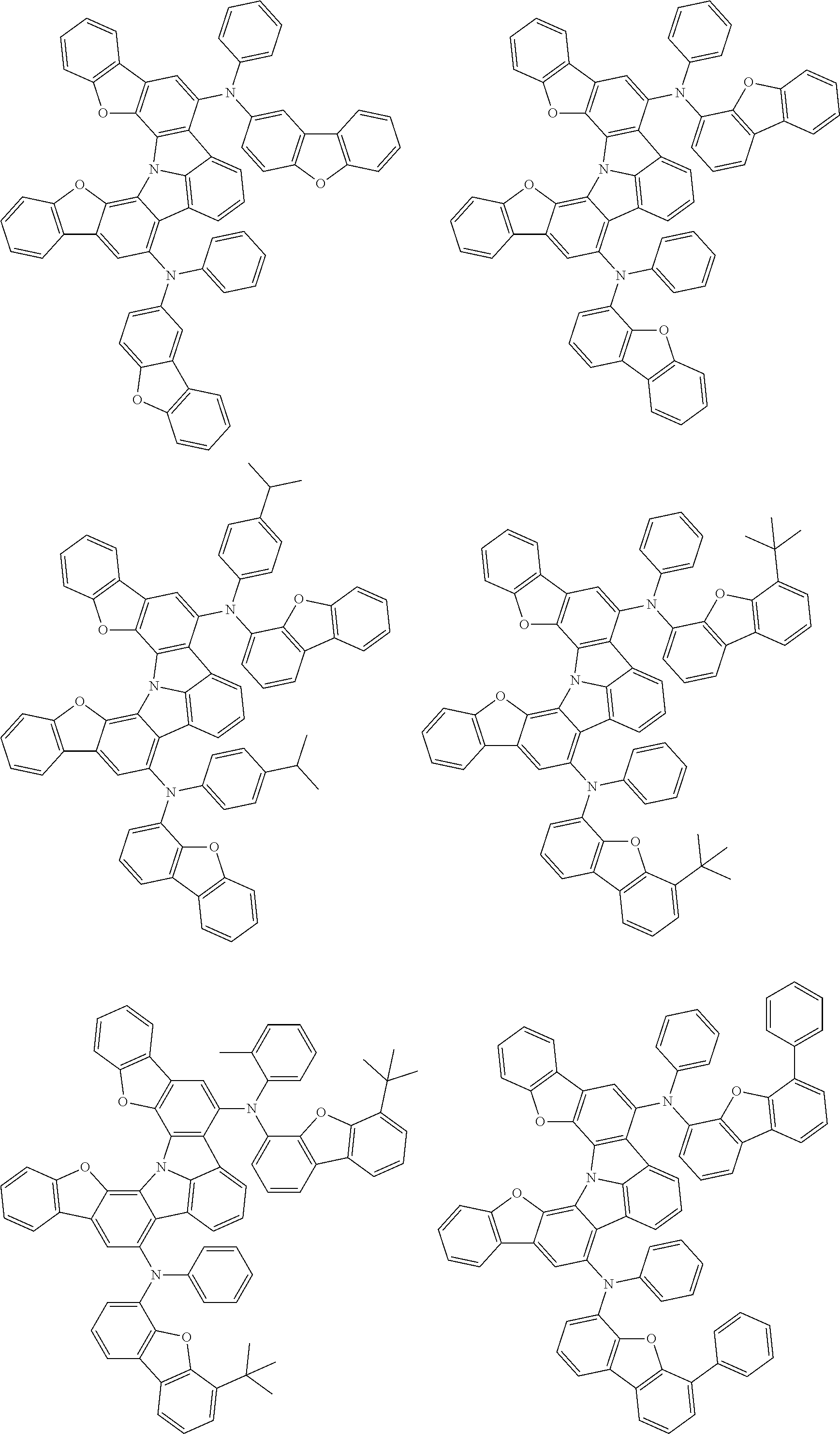

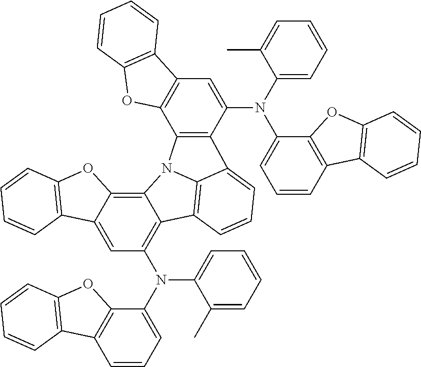 [SMILES: Cc1ccccc1N(c1cccc2c1oc1ccccc12)c1cc2c3ccccc3oc2c2c1c1cccc3c4c(N(c5ccccc5C)c5cccc6c5oc5ccccc56)cc5c6ccccc6oc5c4n2c13]

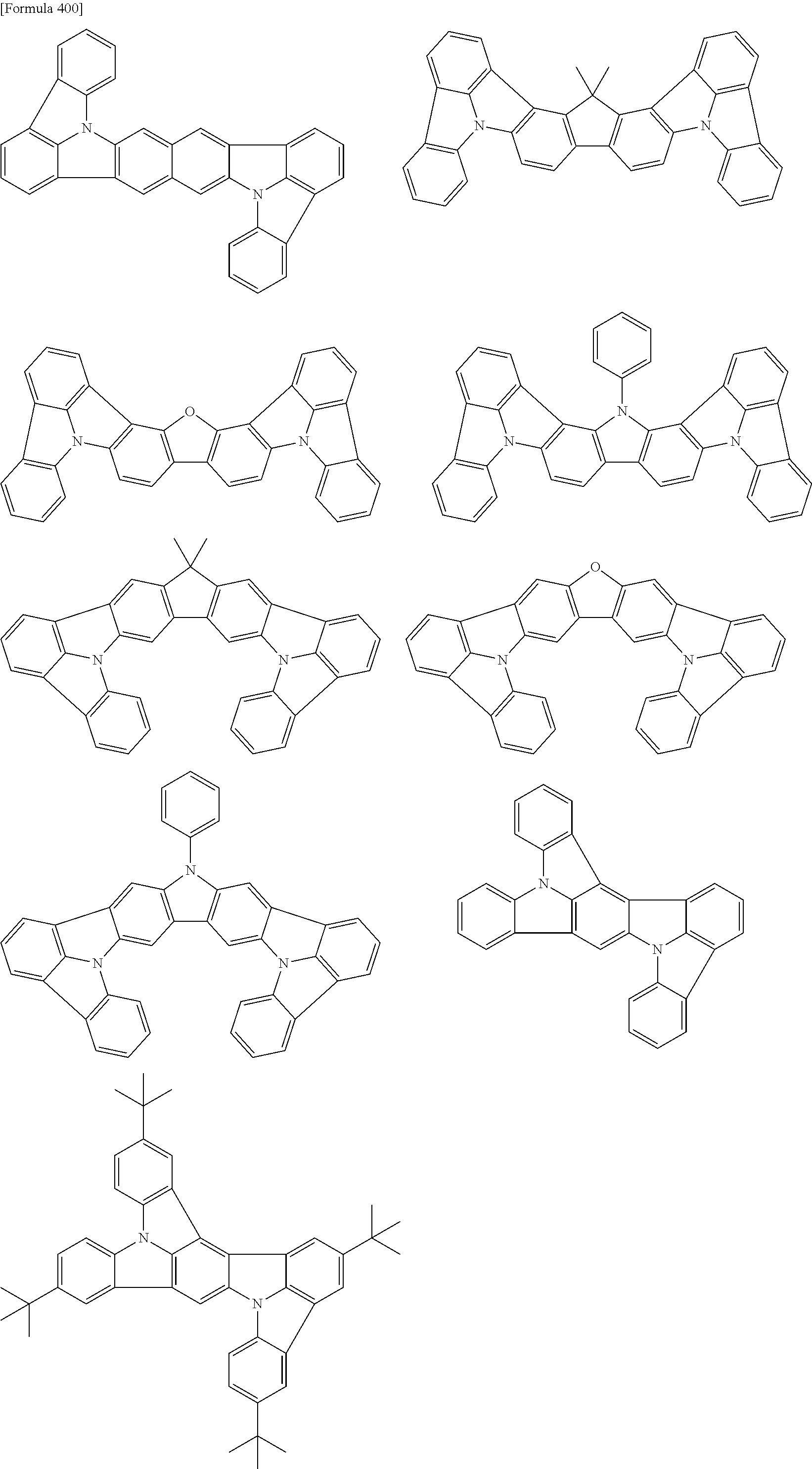

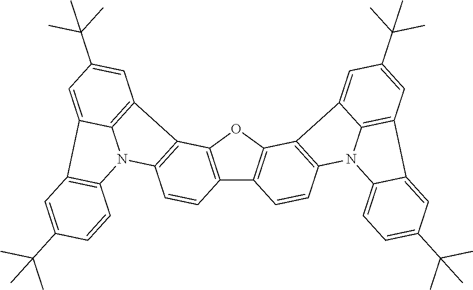 [SMILES: CC(C)(C)c1ccc2c(c1)c1cc(C(C)(C)C)cc3c4c5oc6c(ccc7c6c6cc(C(C)(C)C)cc8c9cc(C(C)(C)C)ccc9n7c86)c5ccc4n2c13]

Compound Represented by Formula (5)

The compound represented by the formula (5) will be described. The compound represented by the formula (5) corresponds to a compound represented by the formula (41-3).

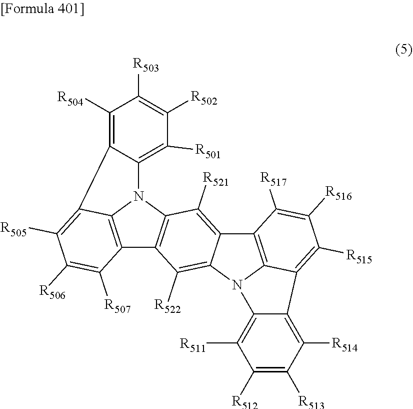

In the formula (5):
at least one combination of adjacent two or more of R₅₀₁ to R₅₀₇ and R₅₁₁ to R₅₁₇ are mutually bonded to form a substituted or unsubstituted monocyclic ring, mutually bonded to form a substituted or unsubstituted fused ring, or not mutually bonded;
R₅₀₁ to R₅₀₇ and R₅₁₁ to R₅₁₇ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group Represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms; and
R₅₂₁ and R₅₂₂ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group Represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

“A combination of adjacent two or more of R₅₀₁ to R₅₀₇ and R₅₁₁ to R₅₁₇” refers to, for instance, a combination of R₅₀₁ and R₅₀₂, a combination of R₅₀₂ and R₅₀₃, a combination of R₅₀₃ and R₅₀₄, a combination of R₅₀₅ and R₅₀₆, a combination of R₅₀₆ and R₅₀₇, and a combination of R₅₀₁, R₅₀₂, and R₅₀₃.

In an exemplary embodiment, at least one, preferably two of R₅₀₁ to R₅₀₇ and R₅₁₁ to R₅₁₇ are groups represented by —N(R₉₀₆)(R₉₀₇).

In an exemplary embodiment, R₅₀₁ to R₅₀₇ and R₅₁₁ to R₅₁₇ are each independently a hydrogen atom, a substituted or unsubstituted aryl group having 6 to 50 ring carbon atoms, or a substituted or unsubstituted heterocyclic group having 5 to 50 ring atoms.

In an exemplary embodiment, a compound represented by the formula (5) is a compound represented by a formula (52) below.

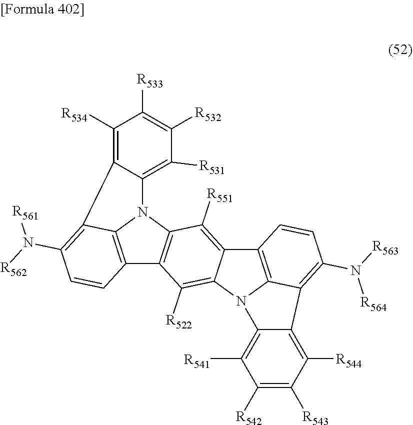

In the formula (52):
at least one combination of adjacent two or more of R₅₃₁ to R₅₃₄ and R₅₄₁ to R₅₄₄ are mutually bonded to form a substituted or unsubstituted monocyclic ring, mutually bonded to form a substituted or unsubstituted fused ring, or not mutually bonded;
R₅₃₁ to R₅₃₄ and R₅₄₁ to R₅₄₄ forming neither the substituted or unsubstituted monocyclic ring nor the substituted or unsubstituted fused ring, and R₅₅₁ and R₅₅₂ are each independently a hydrogen atom, a substituted or unsubstituted aryl group having 6 to 50 ring carbon atoms, or a substituted or unsubstituted heterocyclic group having 5 to 50 ring atoms; and
R₅₆₁ to R₅₆₄ are each independently a substituted or unsubstituted aryl group having 6 to 50 ring carbon atoms, or a substituted or unsubstituted heterocyclic group having 5 to 50 ring atoms.

In an exemplary embodiment, a compound represented by the formula (5) is a compound represented by a formula (53) below.

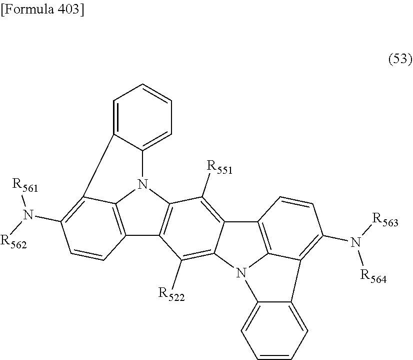

In the formula (53), R₅₅₁, R₅₅₂ and R₅₆₁ to R₅₆₄ each independently represent the same as R₅₅₁, R₅₅₂ and R₅₆₁ to R₅₆₄ in the formula (52).

In an exemplary embodiment, R₅₆₁ to R₅₆₄ in the formulae (52) and (53) are each independently a substituted or unsubstituted aryl group having 6 to 50 ring carbon atoms (preferably a phenyl group).

In an exemplary embodiment, R₅₂₁ and R₅₂₂ in the formula (5) and R₅₅₁ and R₅₅₂ in the formulae (52) and (53) are hydrogen atoms.

In an exemplary embodiment, the substituent for “substituted or unsubstituted” in the formulae (5), (52) and (53) is a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

Specific examples of a compound represented by the formula (5) include compounds shown below.

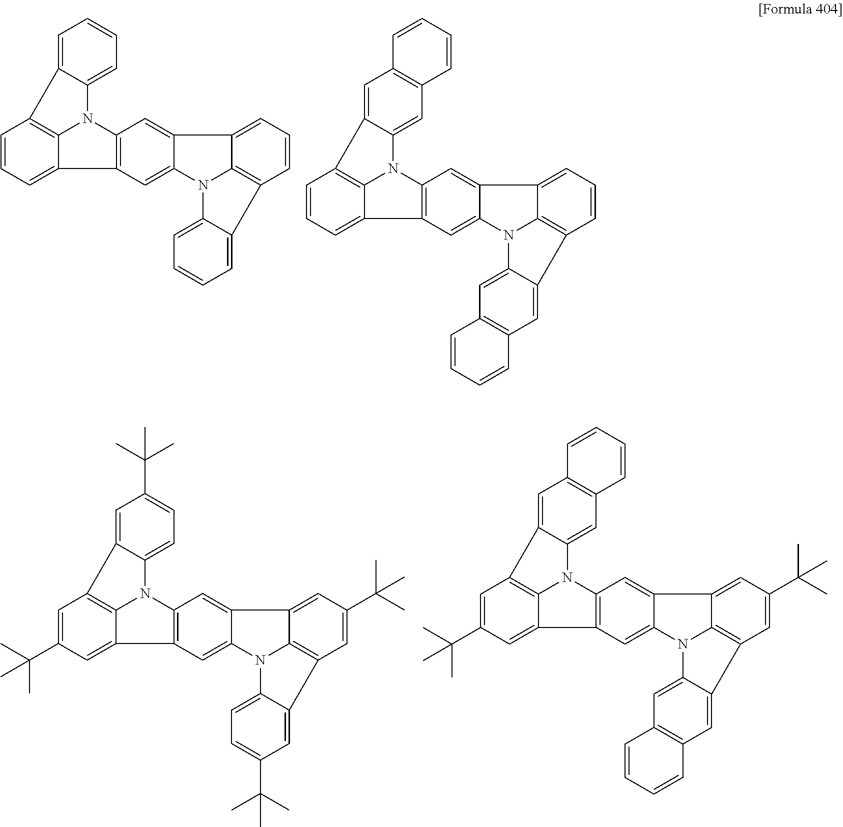

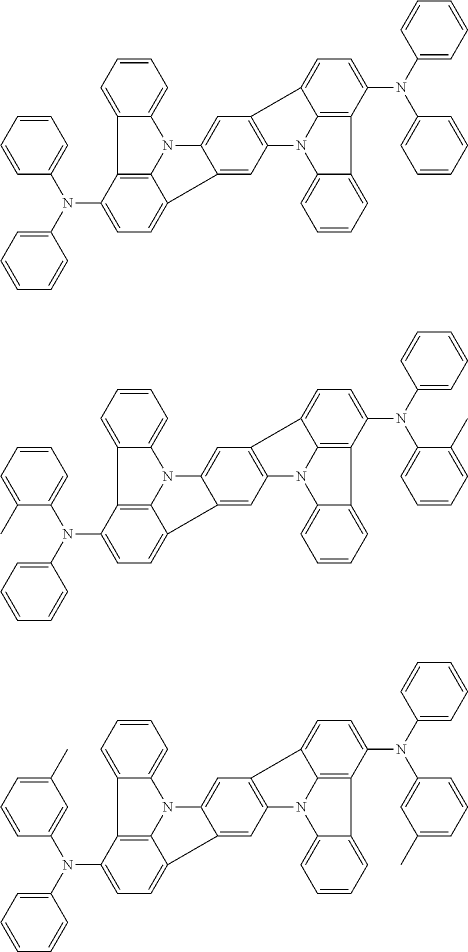 [SMILES: c1ccc(N(c2ccccc2)c2ccc3c4cc5c(cc4n4c6ccccc6c2c34)c2ccc(N(c3ccccc3)c3ccccc3)c3c4ccccc4n5c23)cc1 ; Cc1ccccc1N(c1ccccc1)c1ccc2c3cc4c(cc3n3c5ccccc5c1c23)c1ccc(N(c2ccccc2)c2ccccc2C)c2c3ccccc3n4c12 ; Cc1cccc(N(c2ccccc2)c2ccc3c4cc5c(cc4n4c6ccccc6c2c34)c2ccc(N(c3ccccc3)c3cccc(C)c3)c3c4ccccc4n5c23)c1]

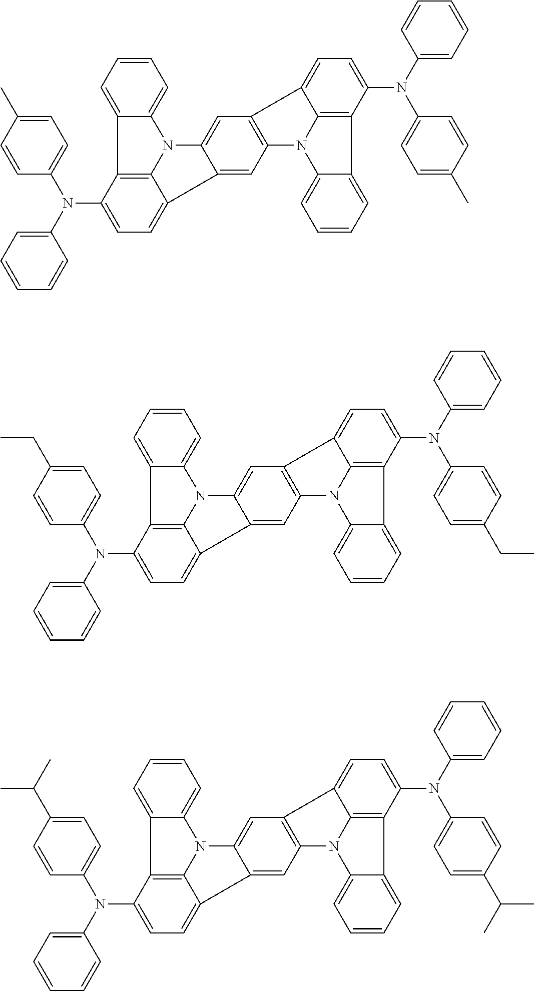

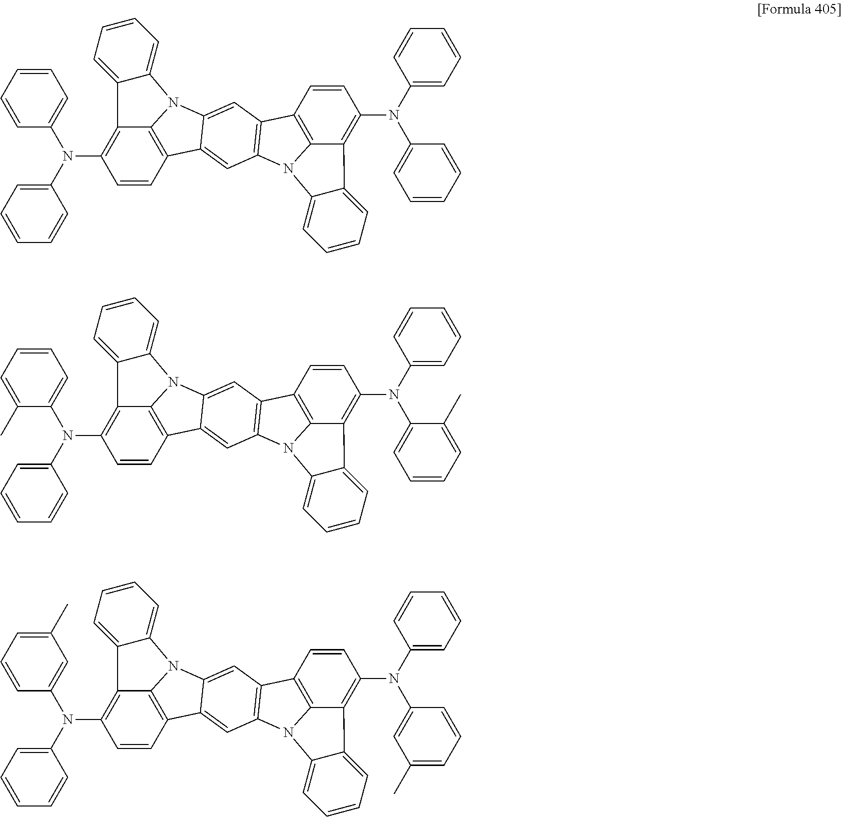 [SMILES: c1ccc(N(c2ccccc2)c2ccc3c4cc5c(cc4n4c6ccccc6c2c34)c2ccc(N(c3ccccc3)c3ccccc3)c3c4ccccc4n5c23)cc1 ; Cc1ccccc1N(c1ccccc1)c1ccc2c3cc4c(cc3n3c5ccccc5c1c23)c1ccc(N(c2ccccc2)c2ccccc2C)c2c3ccccc3n4c12 ; Cc1cccc(N(c2ccccc2)c2ccc3c4cc5c(cc4n4c6ccccc6c2c34)c2ccc(N(c3ccccc3)c3cccc(C)c3)c3c4ccccc4n5c23)c1]

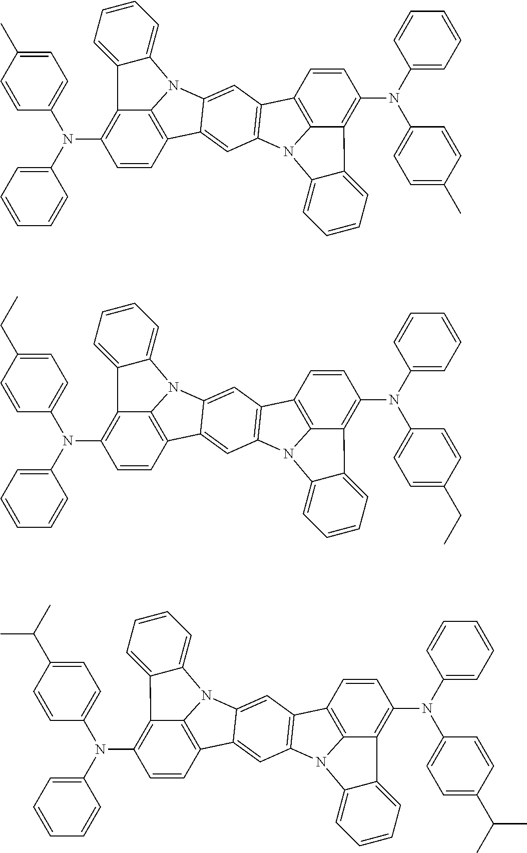

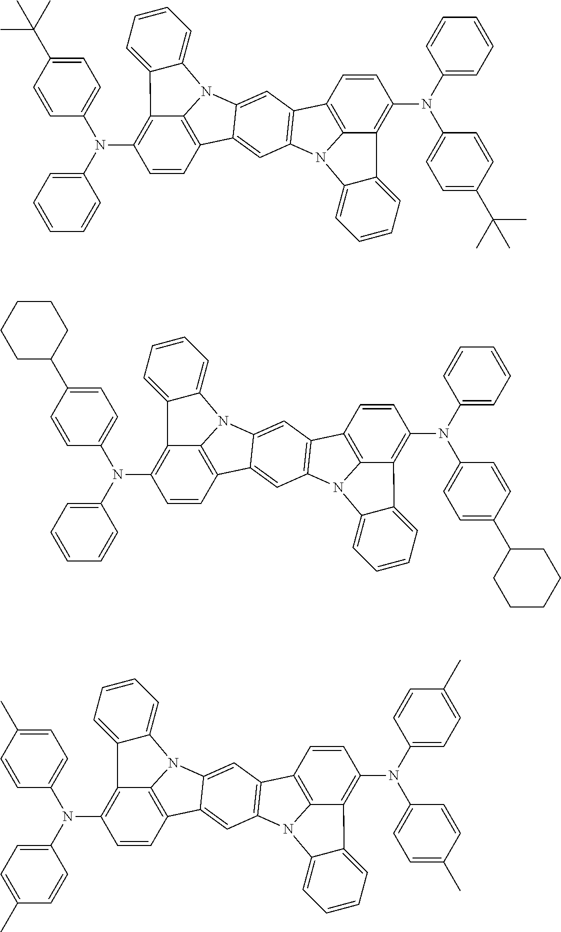 [SMILES: CC(C)(C)c1ccc(N(c2ccccc2)c2ccc3c4cc5c(cc4n4c6ccccc6c2c34)c2ccc(N(c3ccccc3)c3ccc(C(C)(C)C)cc3)c3c4ccccc4n5c23)cc1 ; c1ccc(N(c2ccc(C3CCCCC3)cc2)c2ccc3c4cc5c(cc4n4c6ccccc6c2c34)c2ccc(N(c3ccccc3)c3ccc(C4CCCCC4)cc3)c3c4ccccc4n5c23)cc1 ; Cc1ccc(N(c2ccc(C)cc2)c2ccc3c4cc5c(cc4n4c6ccccc6c2c34)c2ccc(N(c3ccc(C)cc3)c3ccc(C)cc3)c3c4ccccc4n5c23)cc1]

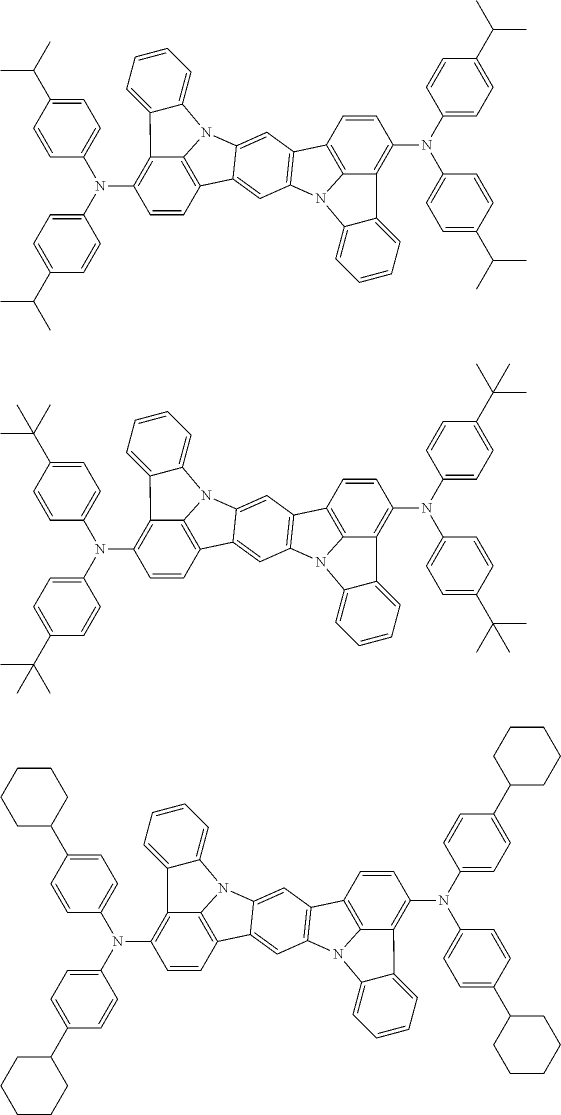

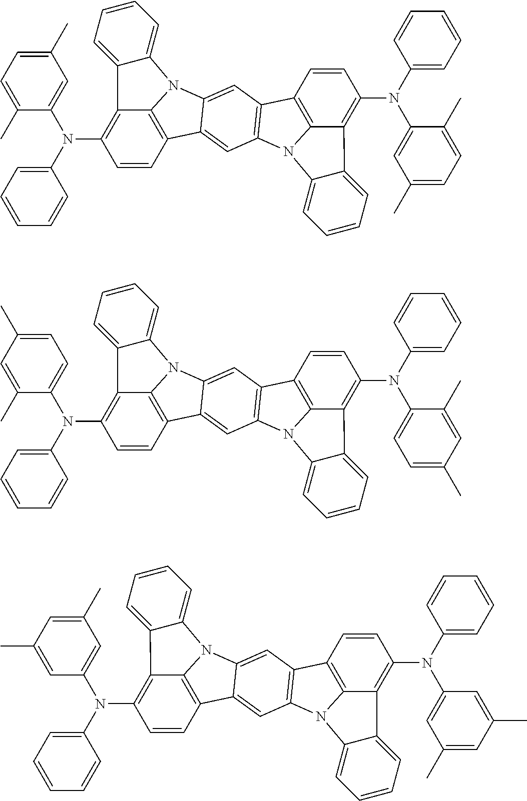

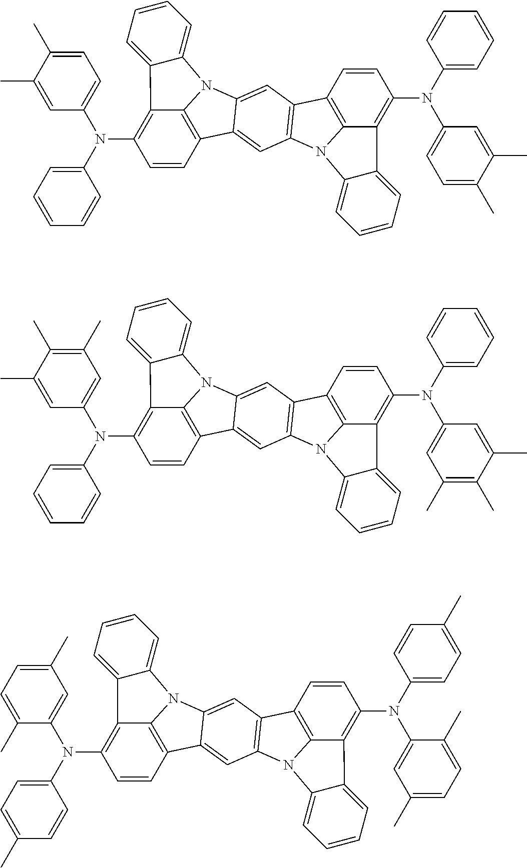

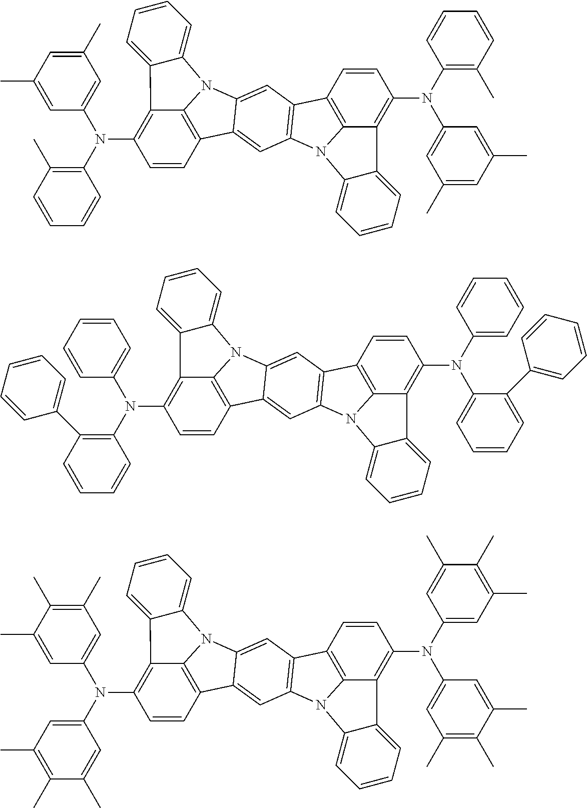

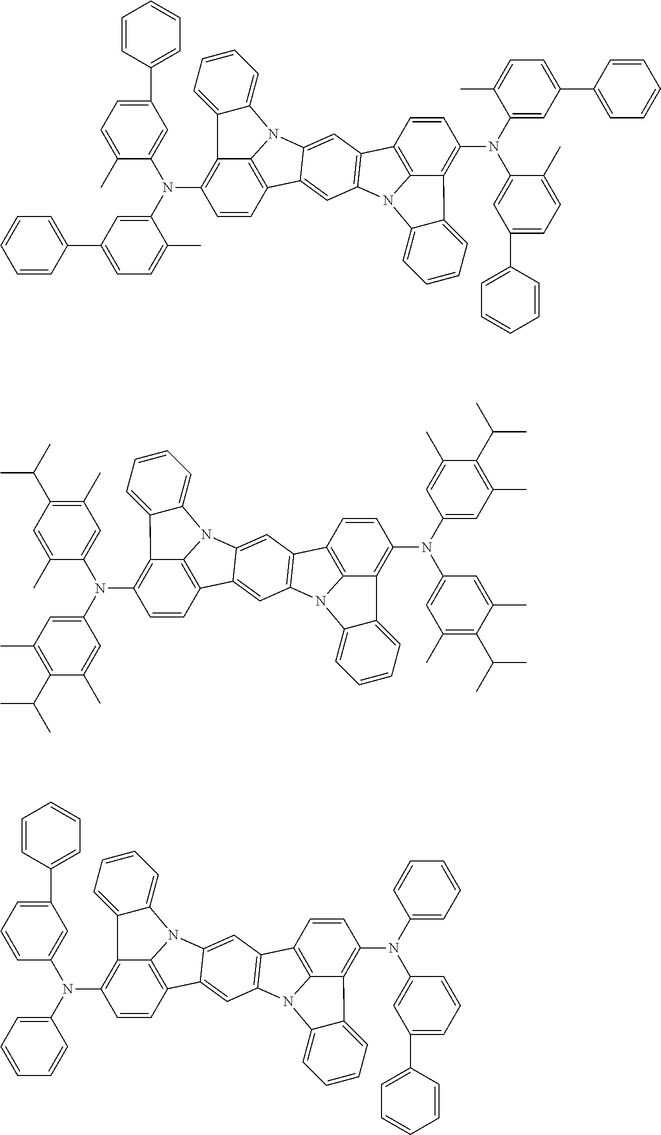 [SMILES: Cc1ccc(-c2ccccc2)cc1N(c1cc(-c2ccccc2)ccc1C)c1ccc2c3cc4c(cc3n3c5ccccc5c1c23)c1ccc(N(c2cc(-c3ccccc3)ccc2C)c2cc(-c3ccccc3)ccc2C)c2c3ccccc3n4c12 ; Cc1cc(N(c2cc(C)c(C(C)C)c(C)c2)c2ccc3c4cc5c(cc4n4c6ccccc6c2c34)c2ccc(N(c3cc(C)c(C(C)C)c(C)c3)c3cc(C)c(C(C)C)c(C)c3)c3c4ccccc4n5c23)c(C)cc1C(C)C ; c1ccc(-c2cccc(N(c3ccccc3)c3ccc4c5cc6c(cc5n5c7ccccc7c3c45)c3ccc(N(c4ccccc4)c4cccc(-c5ccccc5)c4)c4c5ccccc5n6c34)c2)cc1]

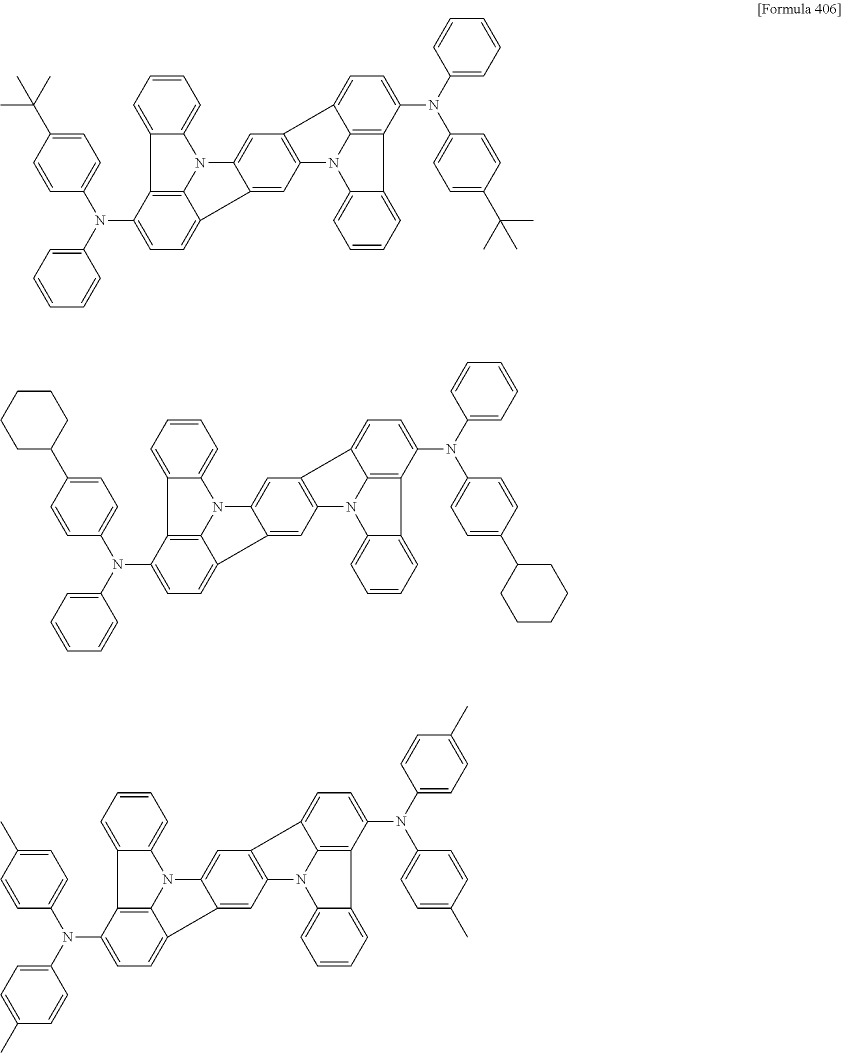 [SMILES: CC(C)(C)c1ccc(N(c2ccccc2)c2ccc3c4cc5c(cc4n4c6ccccc6c2c34)c2ccc(N(c3ccccc3)c3ccc(C(C)(C)C)cc3)c3c4ccccc4n5c23)cc1 ; c1ccc(N(c2ccc(C3CCCCC3)cc2)c2ccc3c4cc5c(cc4n4c6ccccc6c2c34)c2ccc(N(c3ccccc3)c3ccc(C4CCCCC4)cc3)c3c4ccccc4n5c23)cc1 ; Cc1ccc(N(c2ccc(C)cc2)c2ccc3c4cc5c(cc4n4c6ccccc6c2c34)c2ccc(N(c3ccc(C)cc3)c3ccc(C)cc3)c3c4ccccc4n5c23)cc1]

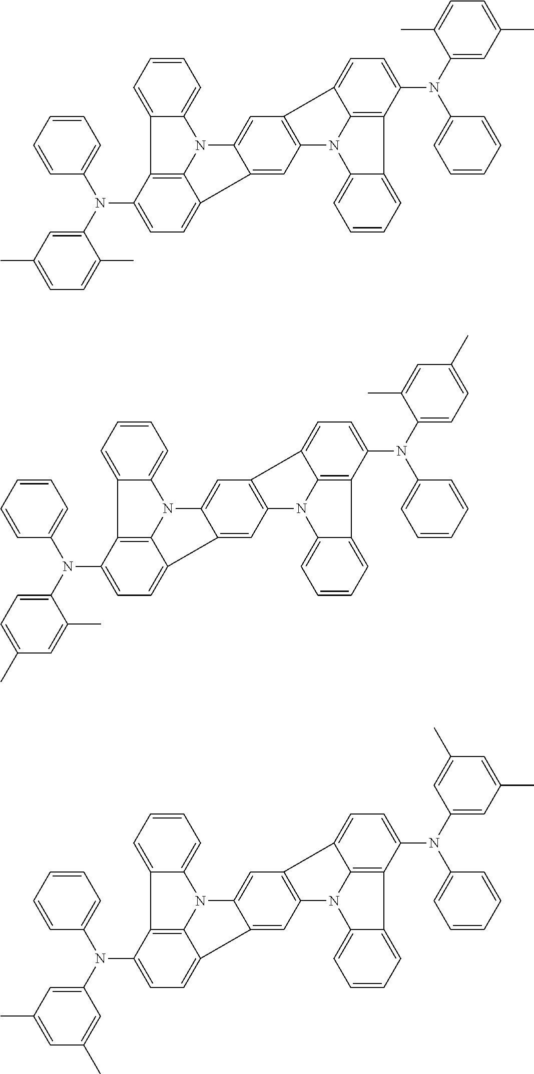

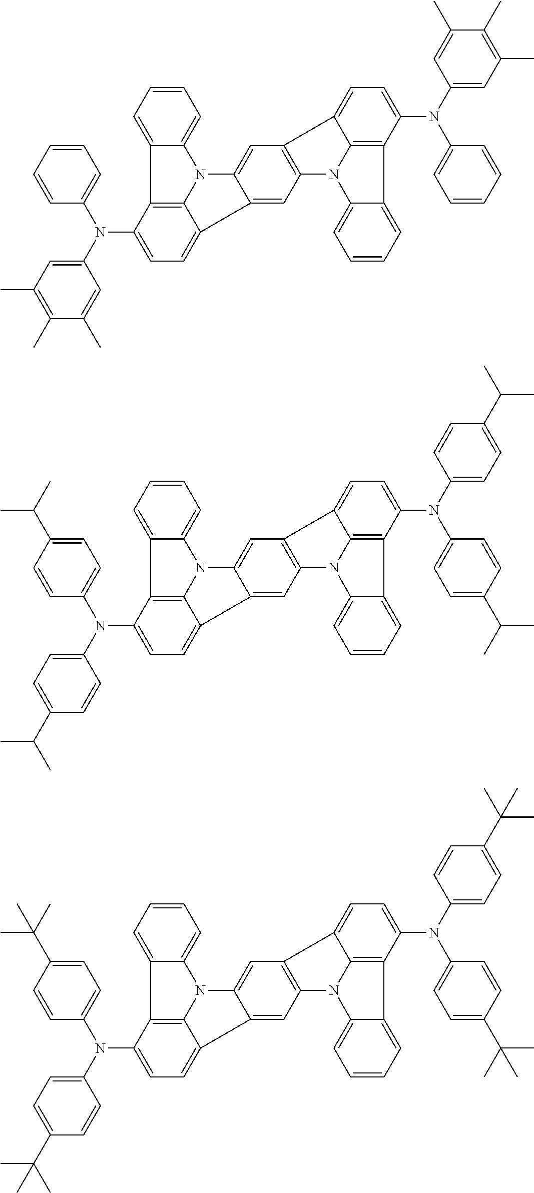

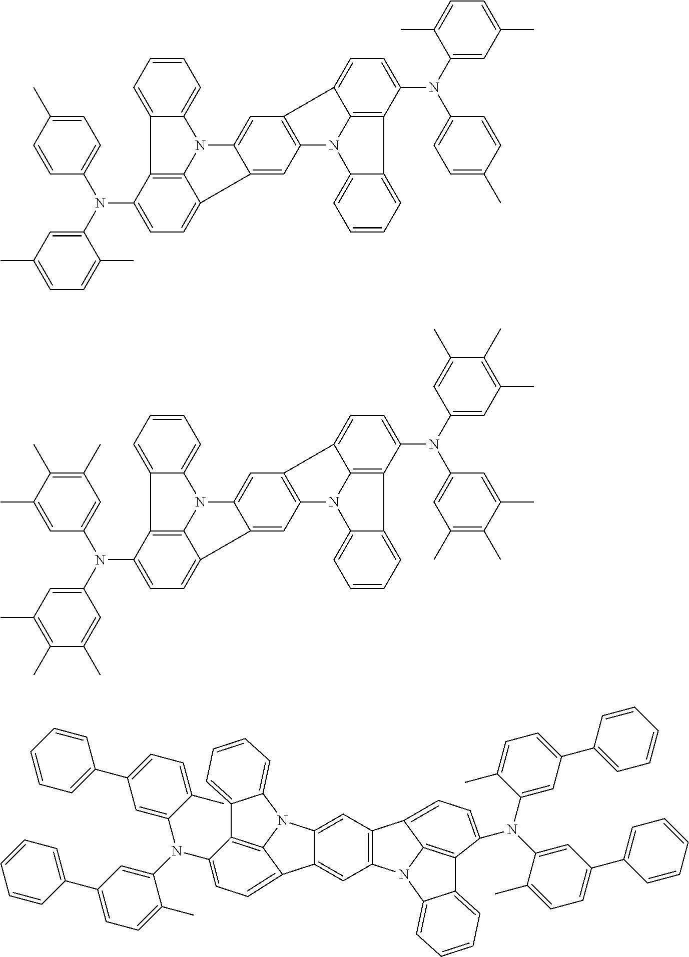

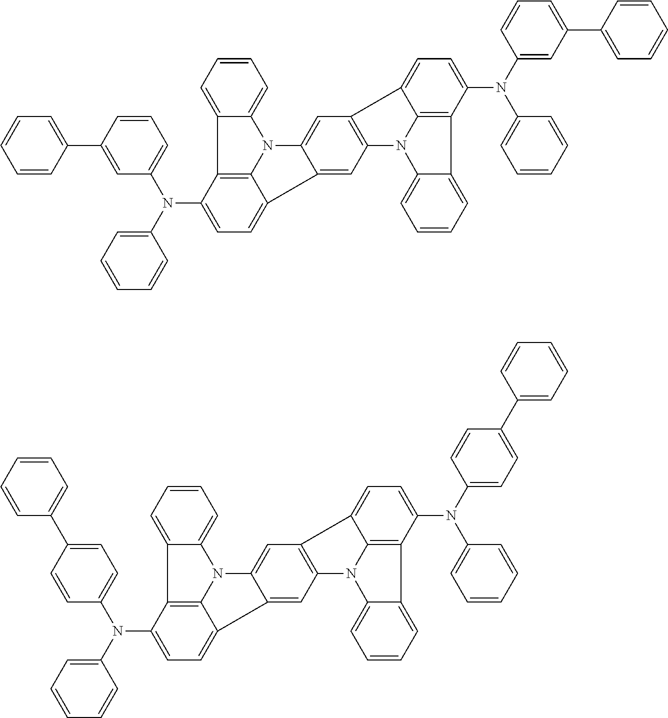 [SMILES: c1ccc(-c2cccc(N(c3ccccc3)c3ccc4c5cc6c(cc5n5c7ccccc7c3c45)c3ccc(N(c4ccccc4)c4cccc(-c5ccccc5)c4)c4c5ccccc5n6c34)c2)cc1 ; c1ccc(-c2ccc(N(c3ccccc3)c3ccc4c5cc6c(cc5n5c7ccccc7c3c45)c3ccc(N(c4ccccc4)c4ccc(-c5ccccc5)cc4)c4c5ccccc5n6c34)cc2)cc1]

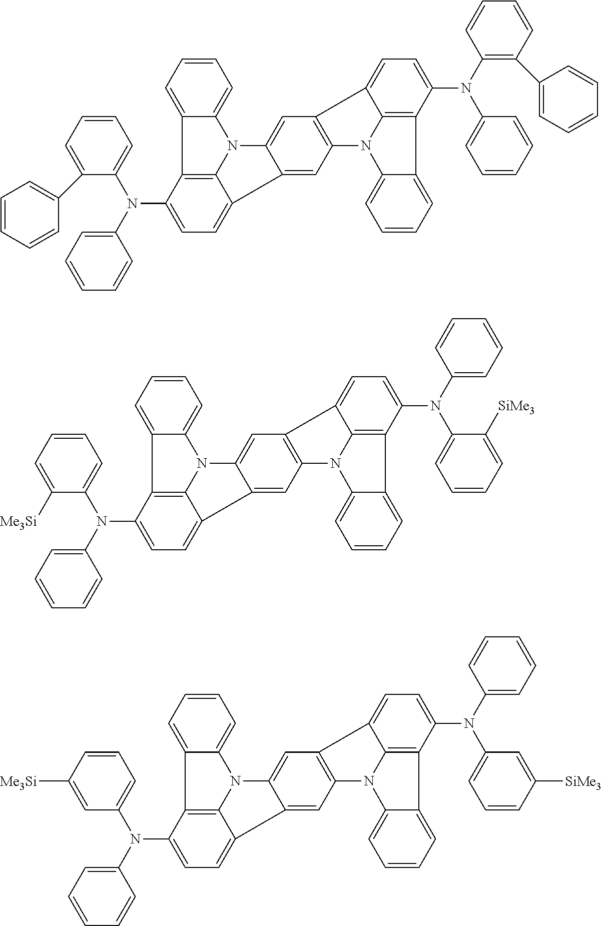 [SMILES: c1ccc(-c2ccccc2N(c2ccccc2)c2ccc3c4cc5c(cc4n4c6ccccc6c2c34)c2ccc(N(c3ccccc3)c3ccccc3-c3ccccc3)c3c4ccccc4n5c23)cc1 ; C[Si](C)(C)c1ccccc1N(c1ccccc1)c1ccc2c3cc4c(cc3n3c5ccccc5c1c23)c1ccc(N(c2ccccc2)c2ccccc2[Si](C)(C)C)c2c3ccccc3n4c12 ; C[Si](C)(C)c1cccc(N(c2ccccc2)c2ccc3c4cc5c(cc4n4c6ccccc6c2c34)c2ccc(N(c3ccccc3)c3cccc([Si](C)(C)C)c3)c3c4ccccc4n5c23)c1]

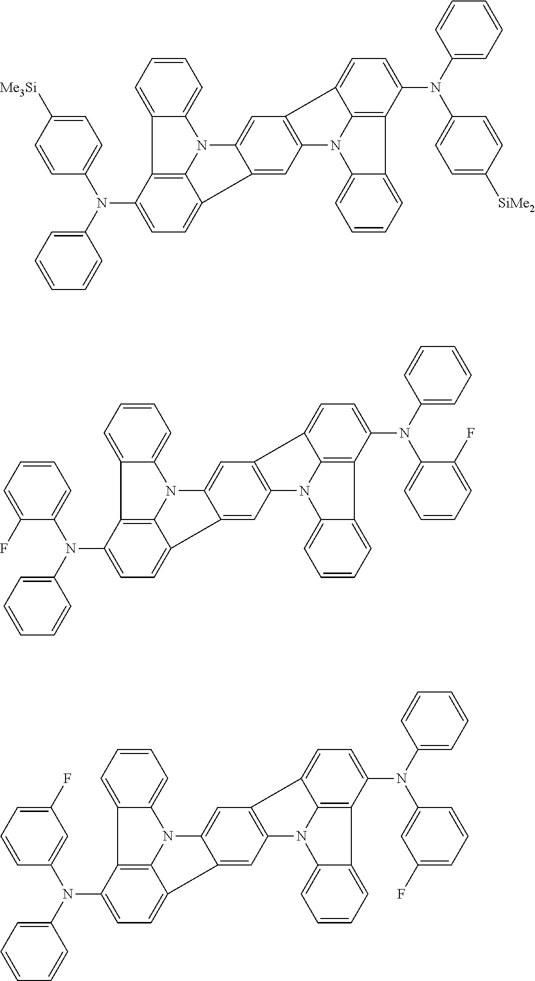

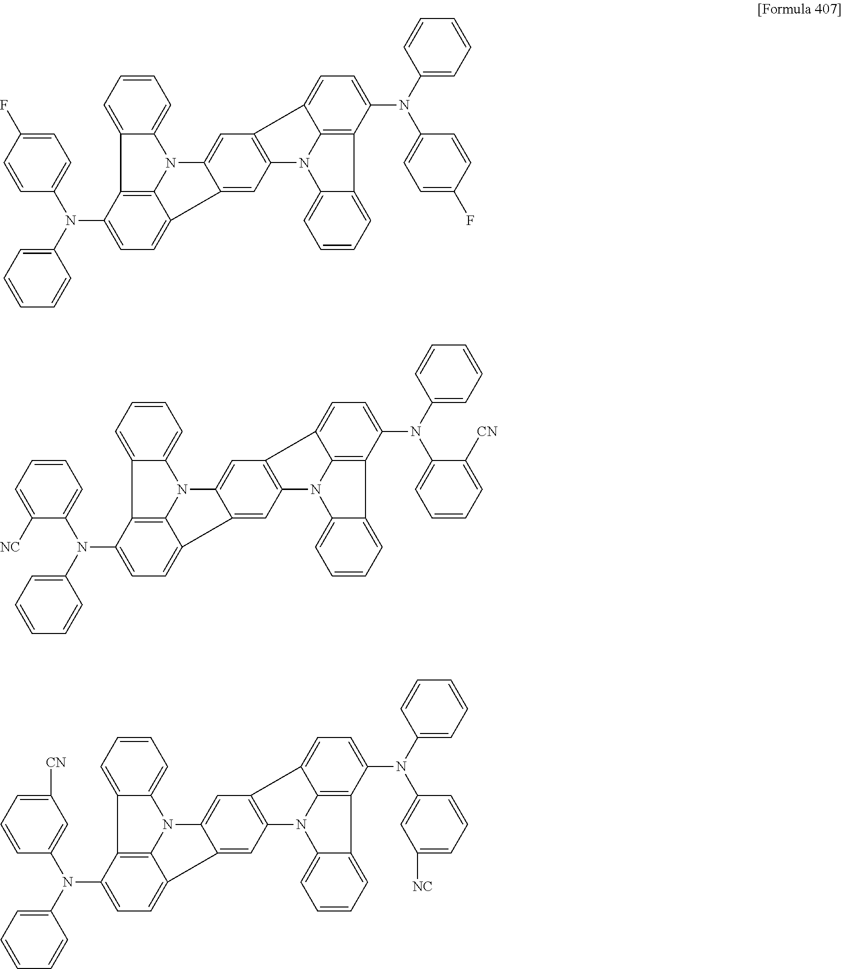

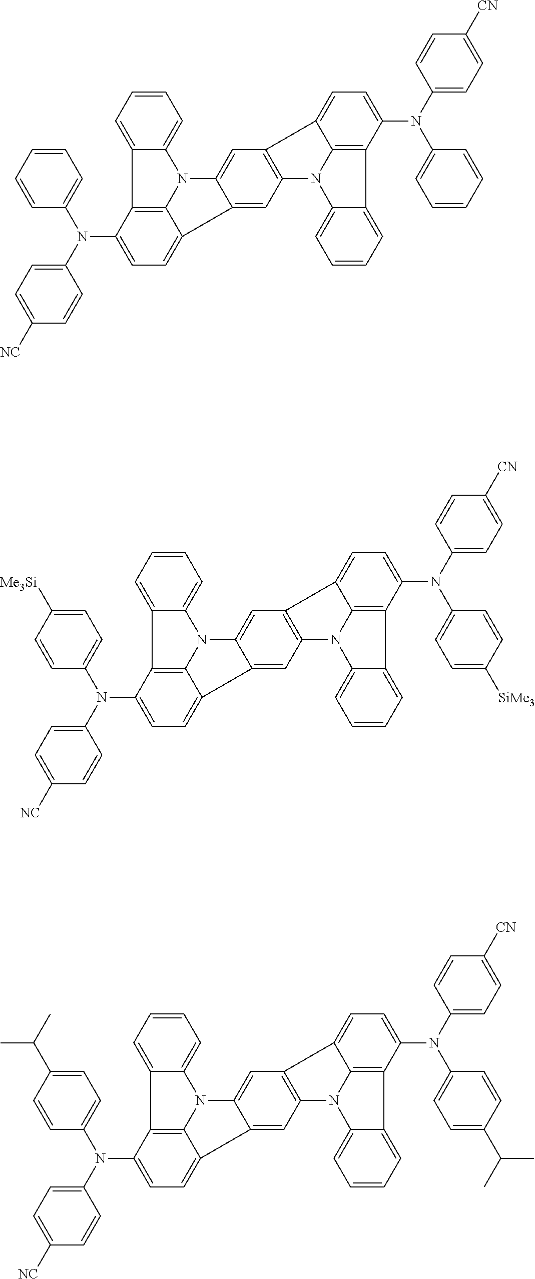 [SMILES: N#Cc1ccc(N(c2ccccc2)c2ccc3c4cc5c(cc4n4c6ccccc6c2c34)c2ccc(N(c3ccccc3)c3ccc(C#N)cc3)c3c4ccccc4n5c23)cc1 ; C[Si](C)(C)c1ccc(N(c2ccc(C#N)cc2)c2ccc3c4cc5c(cc4n4c6ccccc6c2c34)c2ccc(N(c3ccc(C#N)cc3)c3ccc([Si](C)(C)C)cc3)c3c4ccccc4n5c23)cc1 ; CC(C)c1ccc(N(c2ccc(C#N)cc2)c2ccc3c4cc5c(cc4n4c6ccccc6c2c34)c2ccc(N(c3ccc(C#N)cc3)c3ccc(C(C)C)cc3)c3c4ccccc4n5c23)cc1]

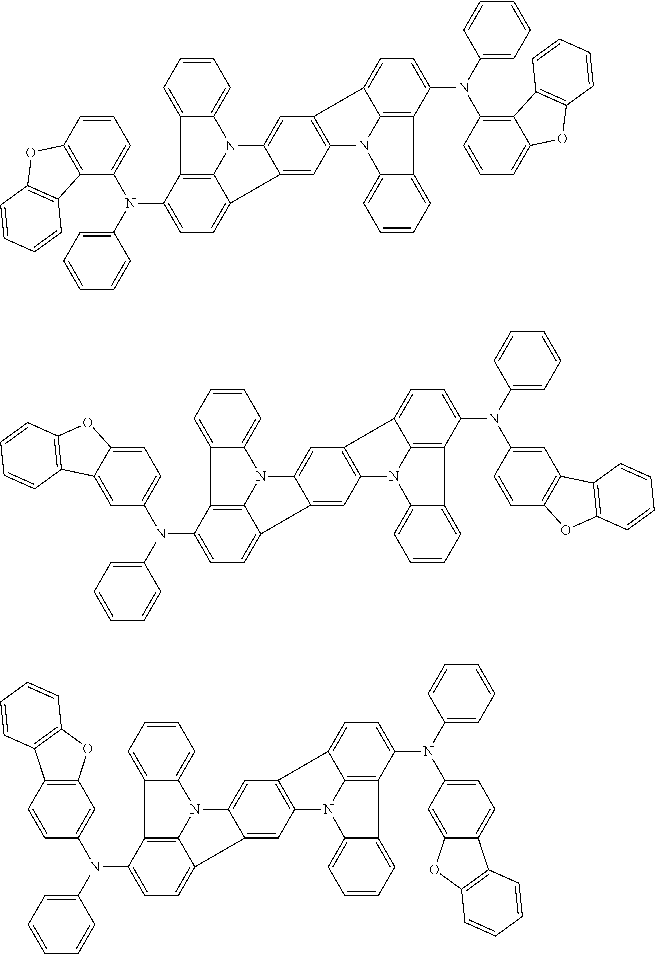 [SMILES: c1ccc(N(c2cccc3oc4ccccc4c23)c2ccc3c4cc5c(cc4n4c6ccccc6c2c34)c2ccc(N(c3ccccc3)c3cccc4oc6ccccc6c34)c3c4ccccc4n5c23)cc1 ; c1ccc(N(c2ccc3oc4ccccc4c3c2)c2ccc3c4cc5c(cc4n4c6ccccc6c2c34)c2ccc(N(c3ccccc3)c3ccc4oc6ccccc6c4c3)c3c4ccccc4n5c23)cc1 ; c1ccc(N(c2ccc3c(c2)oc2ccccc23)c2ccc3c4cc5c(cc4n4c6ccccc6c2c34)c2ccc(N(c3ccccc3)c3ccc4c(c3)oc3ccccc34)c3c4ccccc4n5c23)cc1]

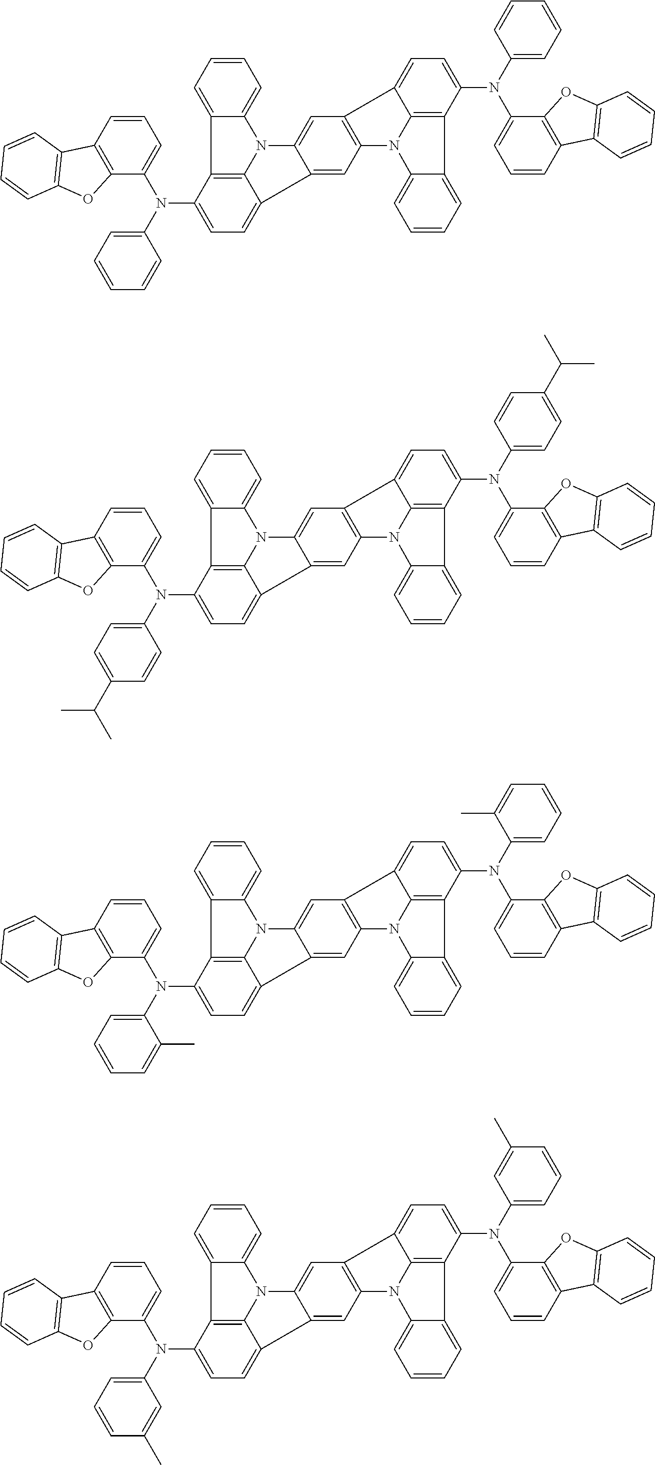 [SMILES: c1ccc(N(c2cccc3c2oc2ccccc23)c2ccc3c4cc5c(cc4n4c6ccccc6c2c34)c2ccc(N(c3ccccc3)c3cccc4c3oc3ccccc34)c3c4ccccc4n5c23)cc1 ; CC(C)c1ccc(N(c2cccc3c2oc2ccccc23)c2ccc3c4cc5c(cc4n4c6ccccc6c2c34)c2ccc(N(c3ccc(C(C)C)cc3)c3cccc4c3oc3ccccc34)c3c4ccccc4n5c23)cc1 ; Cc1ccccc1N(c1cccc2c1oc1ccccc12)c1ccc2c3cc4c(cc3n3c5ccccc5c1c23)c1ccc(N(c2ccccc2C)c2cccc3c2oc2ccccc23)c2c3ccccc3n4c12 ; Cc1cccc(N(c2cccc3c2oc2ccccc23)c2ccc3c4cc5c(cc4n4c6ccccc6c2c34)c2ccc(N(c3cccc(C)c3)c3cccc4c3oc3ccccc34)c3c4ccccc4n5c23)c1]

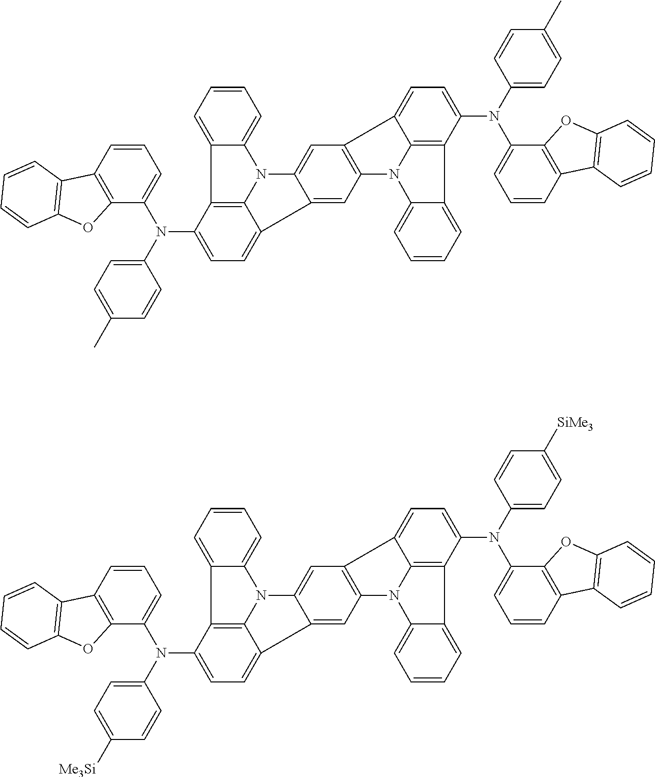 [SMILES: Cc1ccc(N(c2cccc3c2oc2ccccc23)c2ccc3c4cc5c(cc4n4c6ccccc6c2c34)c2ccc(N(c3ccc(C)cc3)c3cccc4c3oc3ccccc34)c3c4ccccc4n5c23)cc1 ; C[Si](C)(C)c1ccc(N(c2cccc3c2oc2ccccc23)c2ccc3c4cc5c(cc4n4c6ccccc6c2c34)c2ccc(N(c3ccc([Si](C)(C)C)cc3)c3cccc4c3oc3ccccc34)c3c4ccccc4n5c23)cc1]

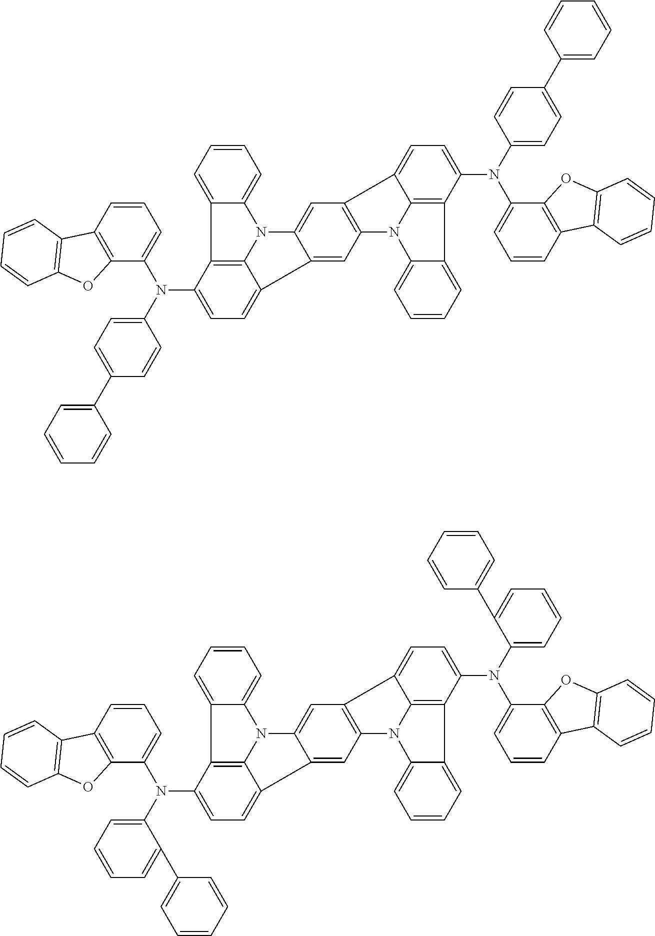 [SMILES: c1ccc(-c2ccc(N(c3cccc4c3oc3ccccc34)c3ccc4c5cc6c(cc5n5c7ccccc7c3c45)c3ccc(N(c4ccc(-c5ccccc5)cc4)c4cccc5c4oc4ccccc45)c4c5ccccc5n6c34)cc2)cc1 ; c1ccc(-c2ccccc2N(c2cccc3c2oc2ccccc23)c2ccc3c4cc5c(cc4n4c6ccccc6c2c34)c2ccc(N(c3ccccc3-c3ccccc3)c3cccc4c3oc3ccccc34)c3c4ccccc4n5c23)cc1]

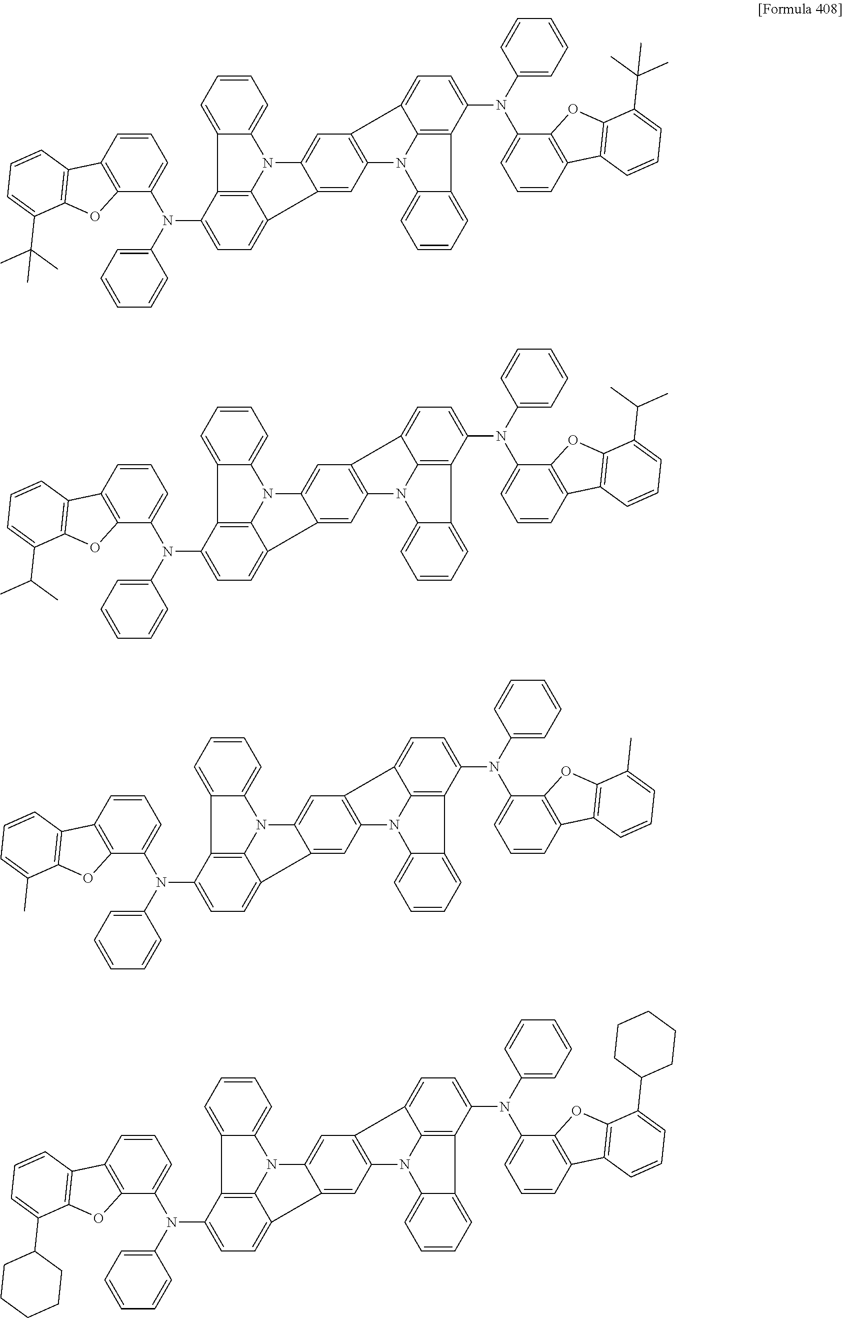

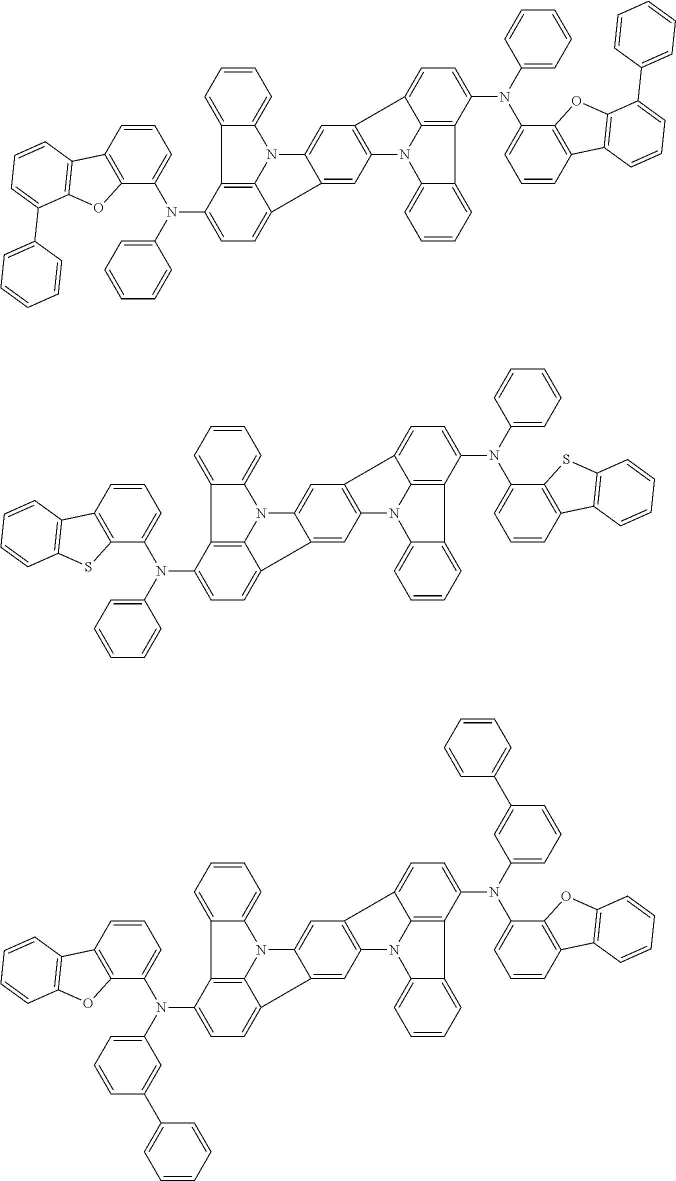 [SMILES: c1ccc(-c2cccc3c2oc2c(N(c4ccccc4)c4ccc5c6cc7c(cc6n6c8ccccc8c4c56)c4ccc(N(c5ccccc5)c5cccc6c5oc5c(-c8ccccc8)cccc56)c5c6ccccc6n7c45)cccc23)cc1 ; c1ccc(N(c2cccc3c2sc2ccccc23)c2ccc3c4cc5c(cc4n4c6ccccc6c2c34)c2ccc(N(c3ccccc3)c3cccc4c3sc3ccccc34)c3c4ccccc4n5c23)cc1 ; c1ccc(-c2cccc(N(c3cccc4c3oc3ccccc34)c3ccc4c5cc6c(cc5n5c7ccccc7c3c45)c3ccc(N(c4cccc(-c5ccccc5)c4)c4cccc5c4oc4ccccc45)c4c5ccccc5n6c34)c2)cc1]

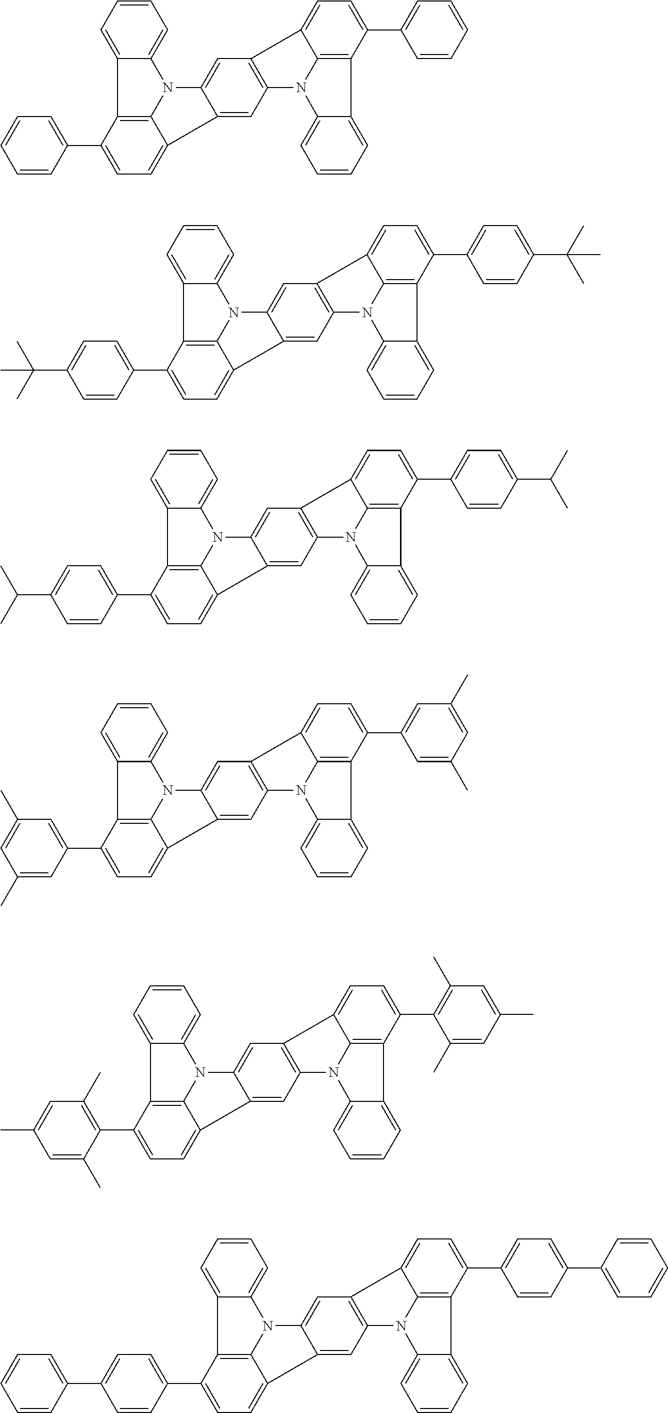

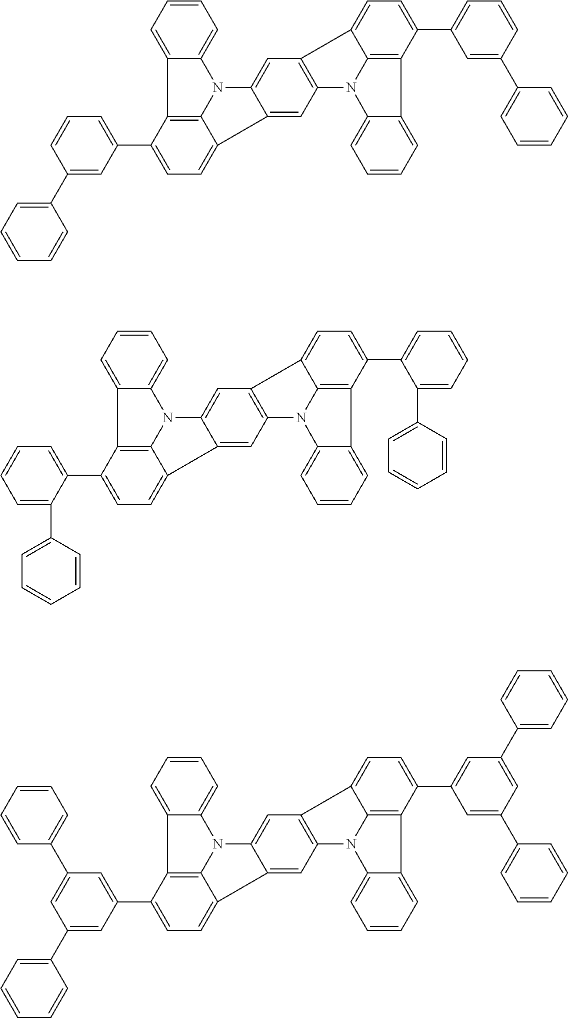 [SMILES: c1ccc(-c2cccc(-c3ccc4c5cc6c(cc5n5c7ccccc7c3c45)c3ccc(-c4cccc(-c5ccccc5)c4)c4c5ccccc5n6c34)c2)cc1 ; c1ccc(-c2ccccc2-c2ccc3c4cc5c(cc4n4c6ccccc6c2c34)c2ccc(-c3ccccc3-c3ccccc3)c3c4ccccc4n5c23)cc1 ; c1ccc(-c2cc(-c3ccccc3)cc(-c3ccc4c5cc6c(cc5n5c7ccccc7c3c45)c3ccc(-c4cc(-c5ccccc5)cc(-c5ccccc5)c4)c4c5ccccc5n6c34)c2)cc1]

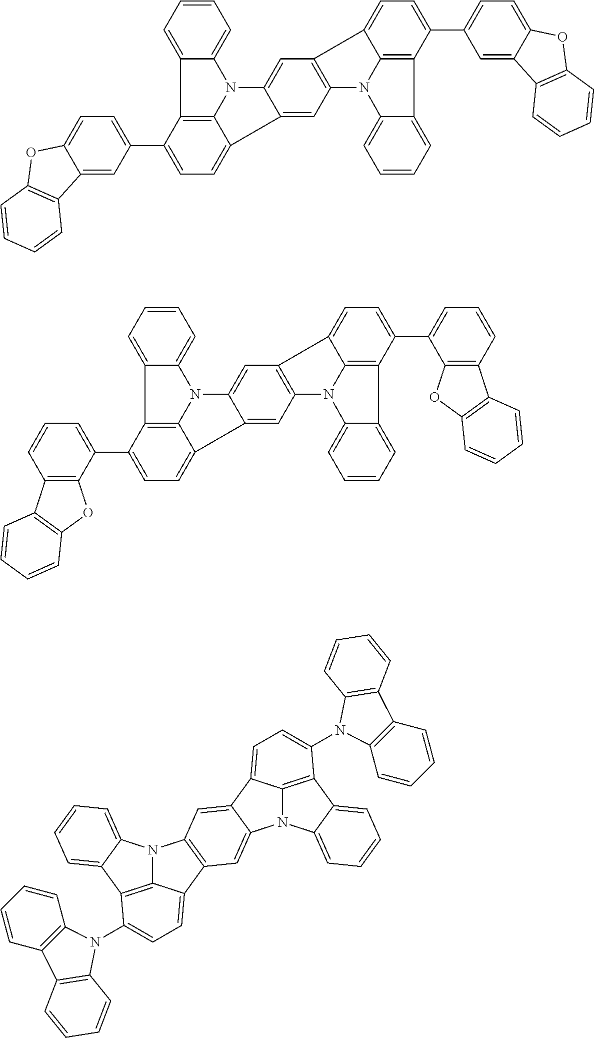 [SMILES: c1ccc2c(c1)oc1ccc(-c3ccc4c5cc6c(cc5n5c7ccccc7c3c45)c3ccc(-c4ccc5oc7ccccc7c5c4)c4c5ccccc5n6c34)cc12 ; c1ccc2c(c1)oc1c(-c3ccc4c5cc6c(cc5n5c7ccccc7c3c45)c3ccc(-c4cccc5c4oc4ccccc45)c4c5ccccc5n6c34)cccc12 ; c1ccc2c(c1)c1ccccc1n2-c1ccc2c3cc4c(cc3n3c5ccccc5c1c23)c1ccc(-n2c3ccccc3c3ccccc32)c2c3ccccc3n4c12]

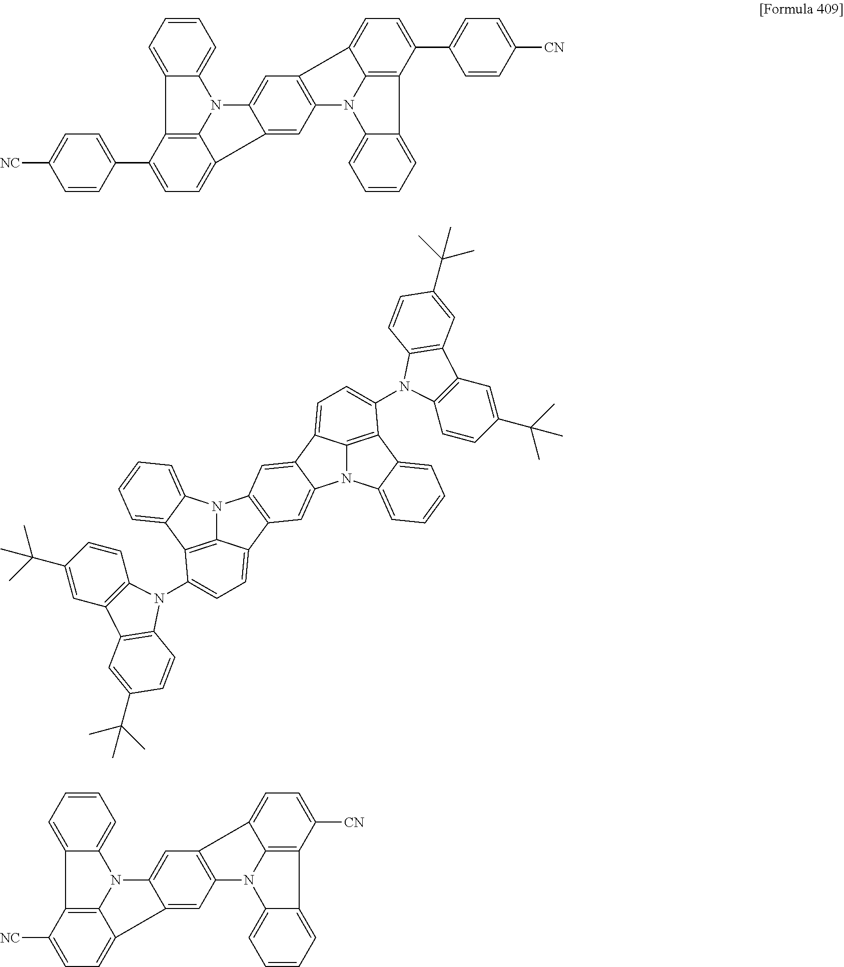

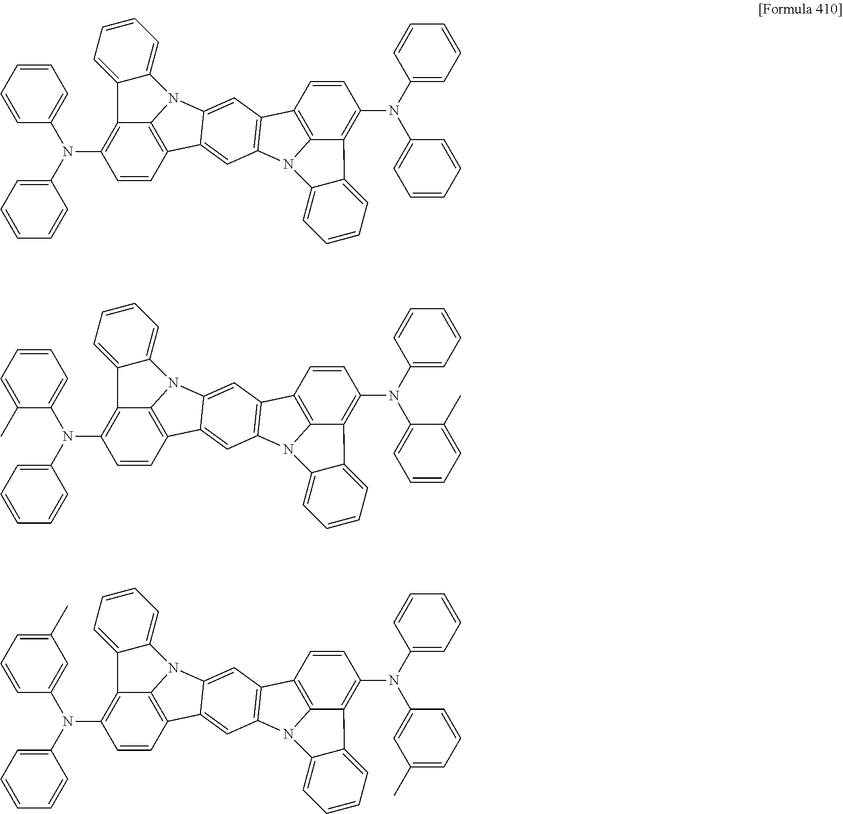 [SMILES: c1ccc(N(c2ccccc2)c2ccc3c4cc5c(cc4n4c6ccccc6c2c34)c2ccc(N(c3ccccc3)c3ccccc3)c3c4ccccc4n5c23)cc1 ; Cc1ccccc1N(c1ccccc1)c1ccc2c3cc4c(cc3n3c5ccccc5c1c23)c1ccc(N(c2ccccc2)c2ccccc2C)c2c3ccccc3n4c12 ; Cc1cccc(N(c2ccccc2)c2ccc3c4cc5c(cc4n4c6ccccc6c2c34)c2ccc(N(c3ccccc3)c3cccc(C)c3)c3c4ccccc4n5c23)c1]

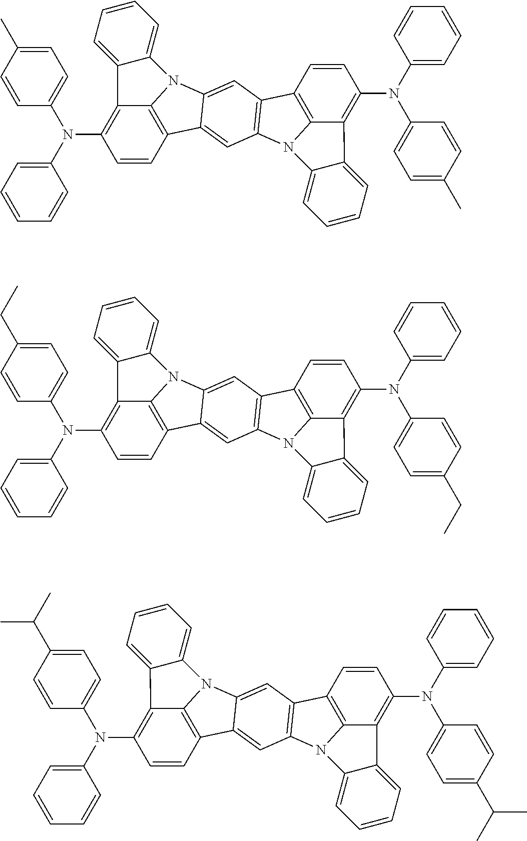

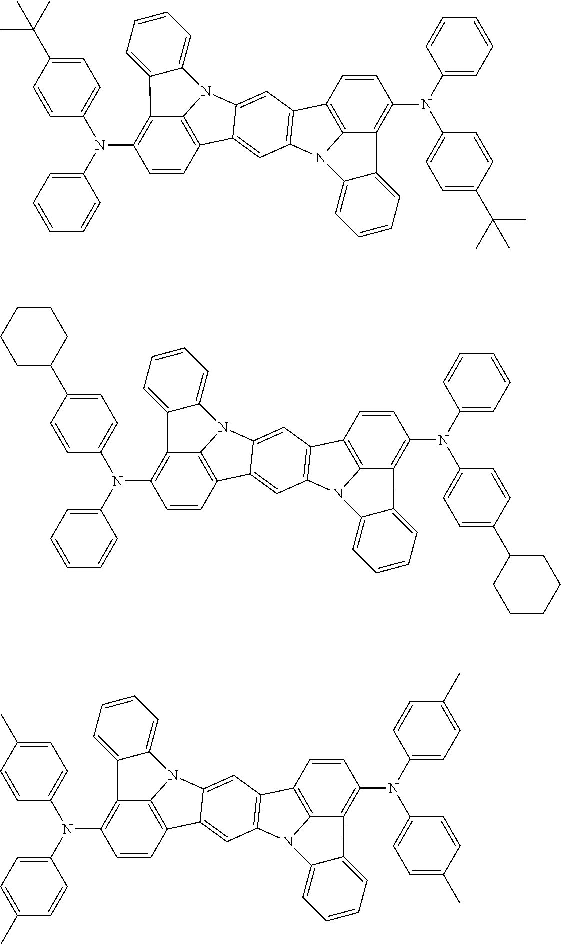 [SMILES: CC(C)(C)c1ccc(N(c2ccccc2)c2ccc3c4cc5c(cc4n4c6ccccc6c2c34)c2ccc(N(c3ccccc3)c3ccc(C(C)(C)C)cc3)c3c4ccccc4n5c23)cc1 ; c1ccc(N(c2ccc(C3CCCCC3)cc2)c2ccc3c4cc5c(cc4n4c6ccccc6c2c34)c2ccc(N(c3ccccc3)c3ccc(C4CCCCC4)cc3)c3c4ccccc4n5c23)cc1 ; Cc1ccc(N(c2ccc(C)cc2)c2ccc3c4cc5c(cc4n4c6ccccc6c2c34)c2ccc(N(c3ccc(C)cc3)c3ccc(C)cc3)c3c4ccccc4n5c23)cc1]

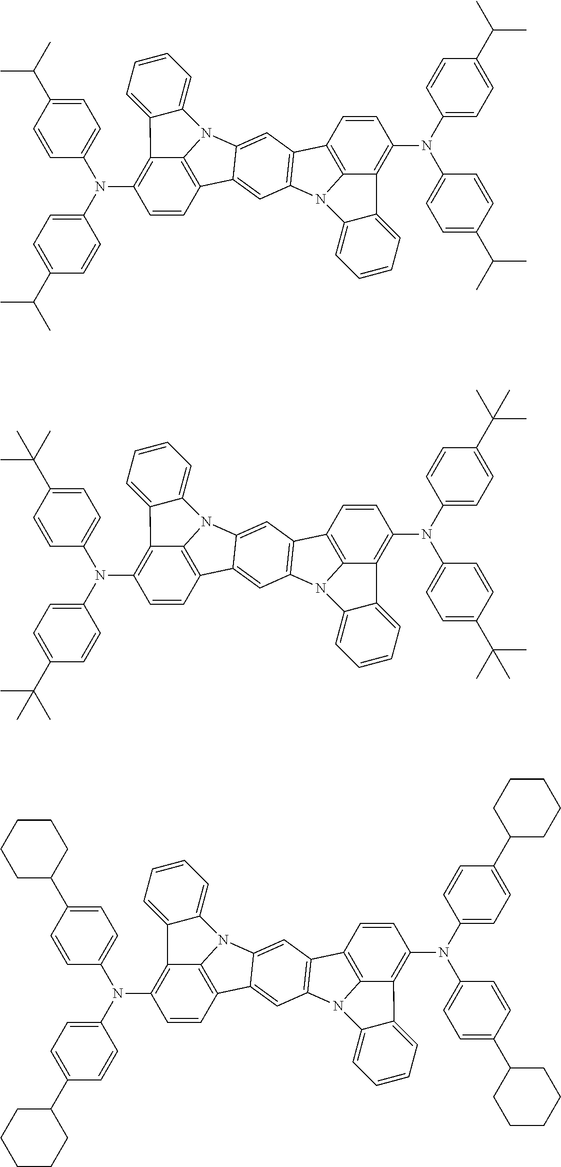

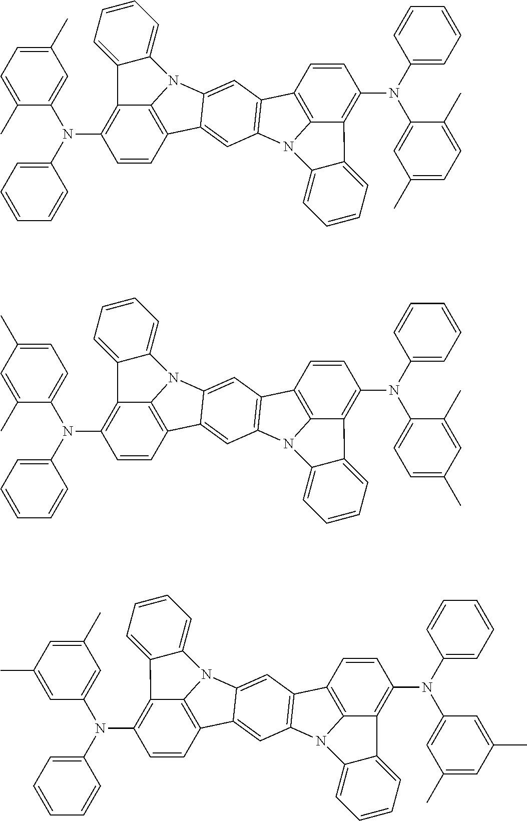

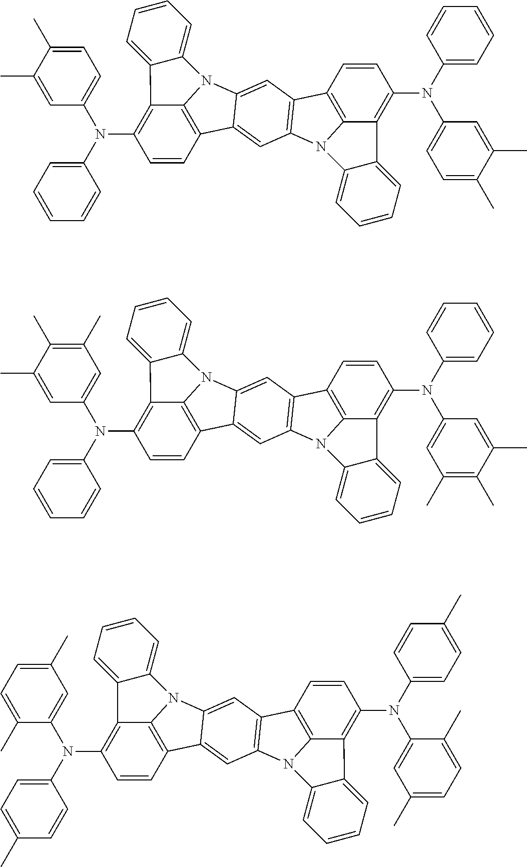

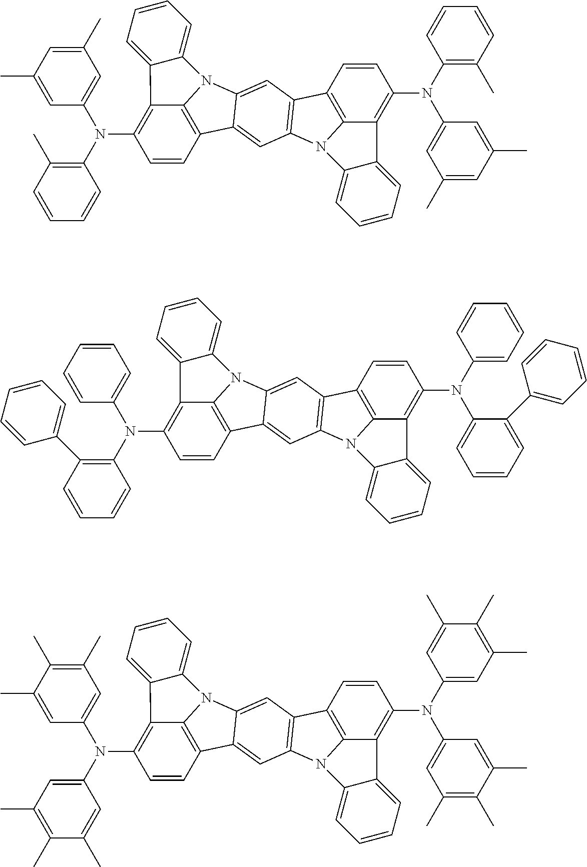

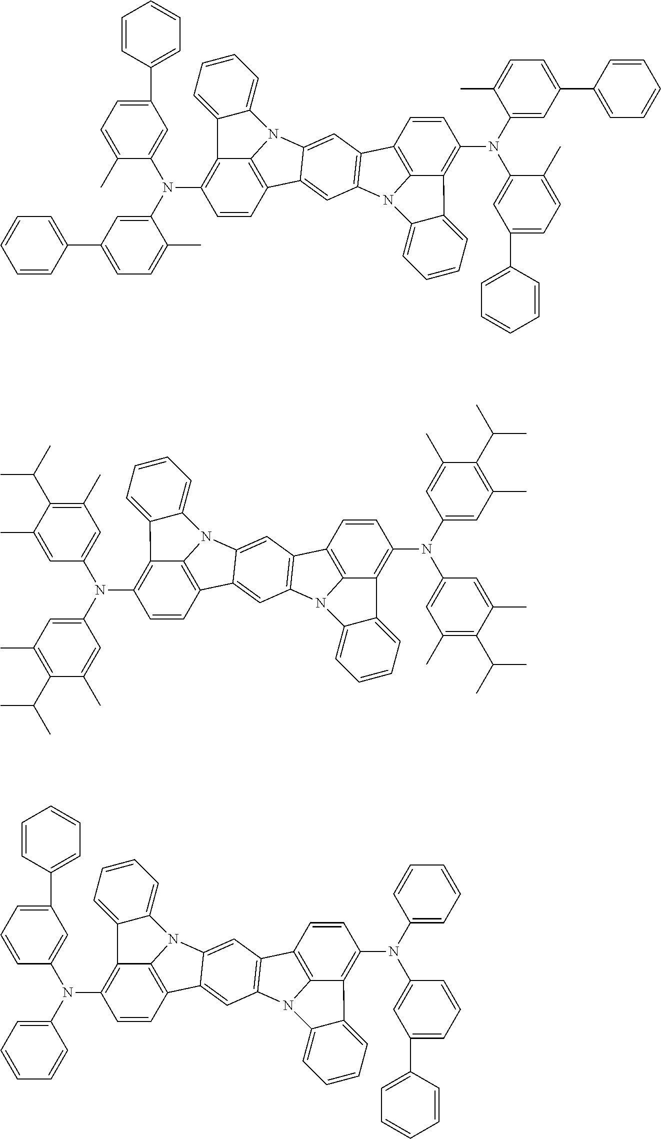 [SMILES: Cc1ccc(-c2ccccc2)cc1N(c1cc(-c2ccccc2)ccc1C)c1ccc2c3cc4c(cc3n3c5ccccc5c1c23)c1ccc(N(c2cc(-c3ccccc3)ccc2C)c2cc(-c3ccccc3)ccc2C)c2c3ccccc3n4c12 ; Cc1cc(N(c2cc(C)c(C(C)C)c(C)c2)c2ccc3c4cc5c(cc4n4c6ccccc6c2c34)c2ccc(N(c3cc(C)c(C(C)C)c(C)c3)c3cc(C)c(C(C)C)c(C)c3)c3c4ccccc4n5c23)cc(C)c1C(C)C ; c1ccc(-c2cccc(N(c3ccccc3)c3ccc4c5cc6c(cc5n5c7ccccc7c3c45)c3ccc(N(c4ccccc4)c4cccc(-c5ccccc5)c4)c4c5ccccc5n6c34)c2)cc1]

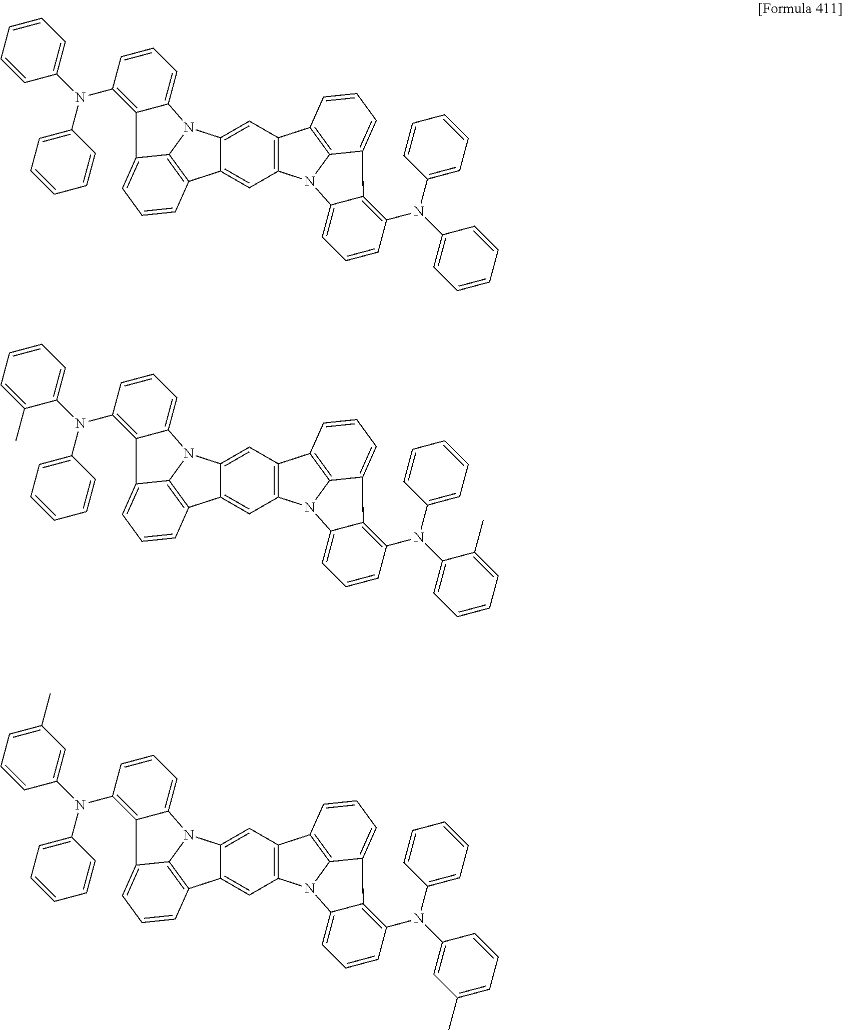 [SMILES: c1ccc(N(c2ccccc2)c2cccc3c2c2cccc4c5cc6c(cc5n3c42)c2cccc3c4c(N(c5ccccc5)c5ccccc5)cccc4n6c23)cc1 ; Cc1ccccc1N(c1ccccc1)c1cccc2c1c1cccc3c4cc5c(cc4n2c31)c1cccc2c3c(N(c4ccccc4)c4ccccc4C)cccc3n5c12 ; Cc1cccc(N(c2ccccc2)c2cccc3c2c2cccc4c5cc6c(cc5n3c42)c2cccc3c4c(N(c5ccccc5)c5cccc(C)c5)cccc4n6c23)c1]

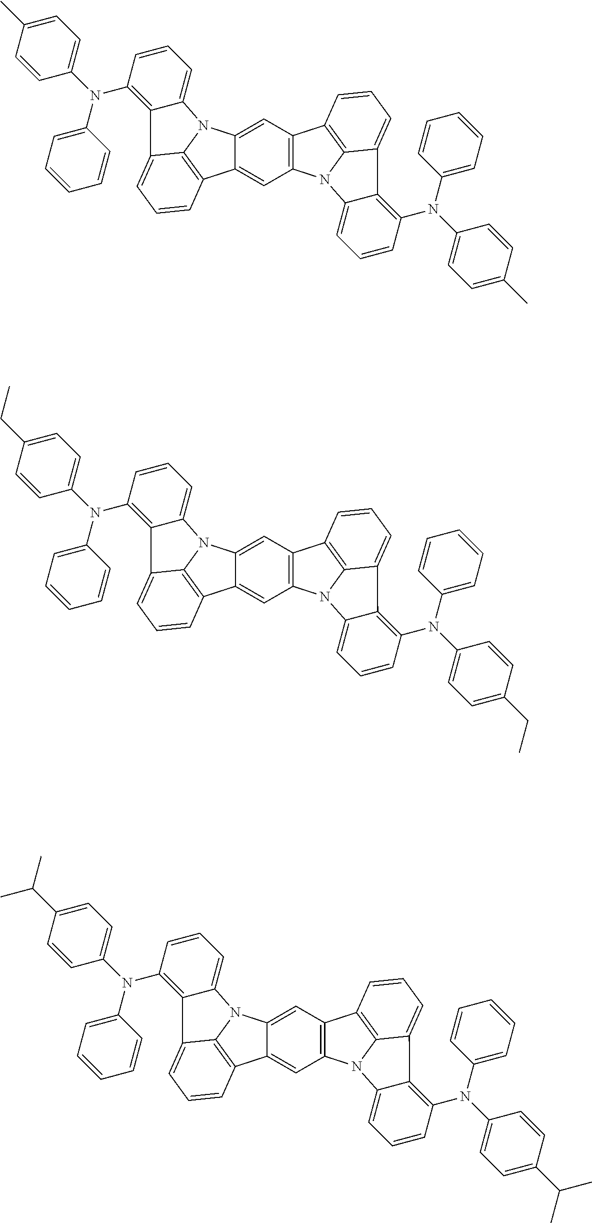

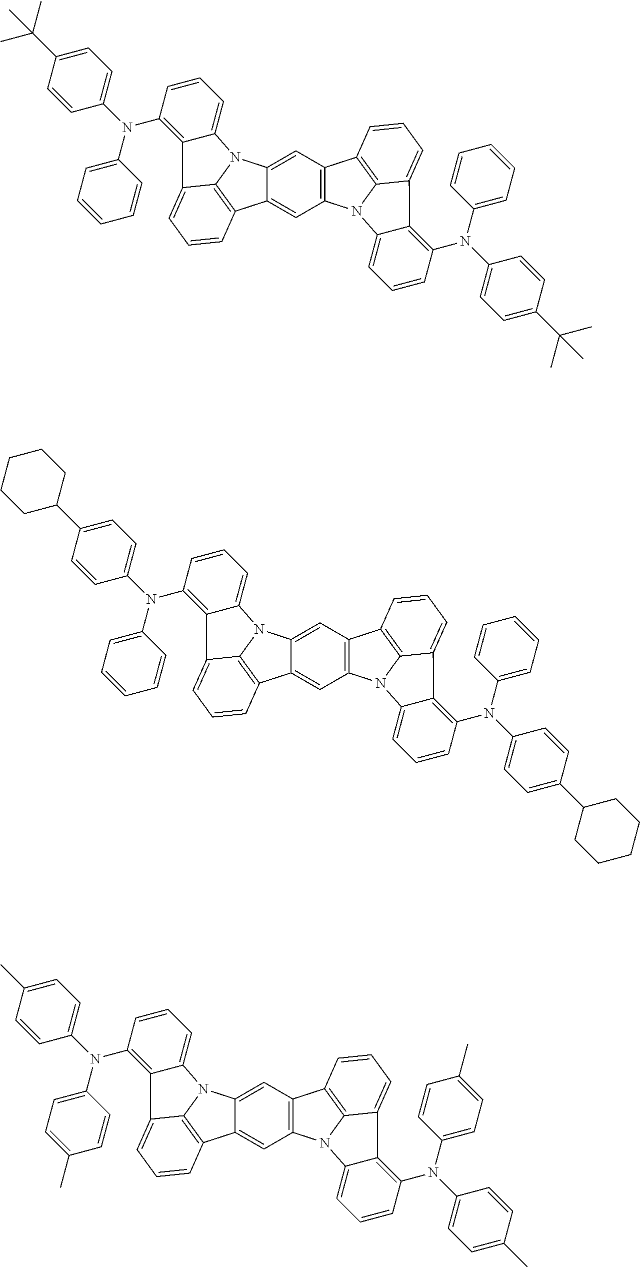

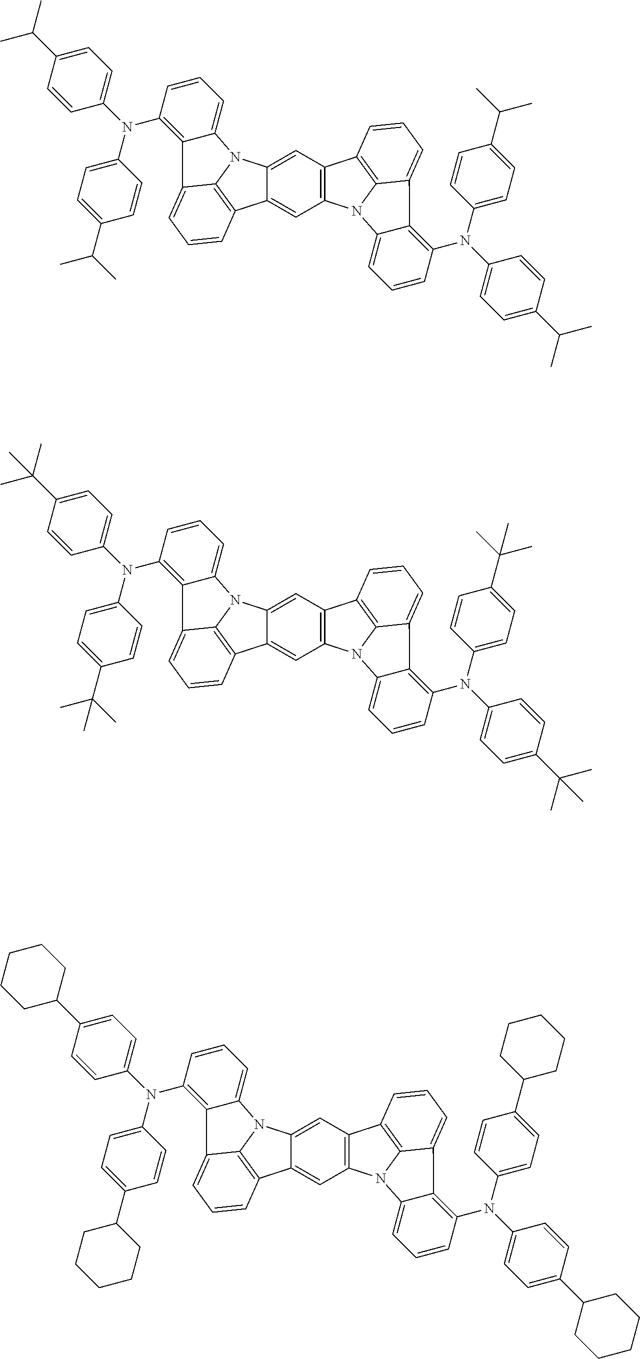

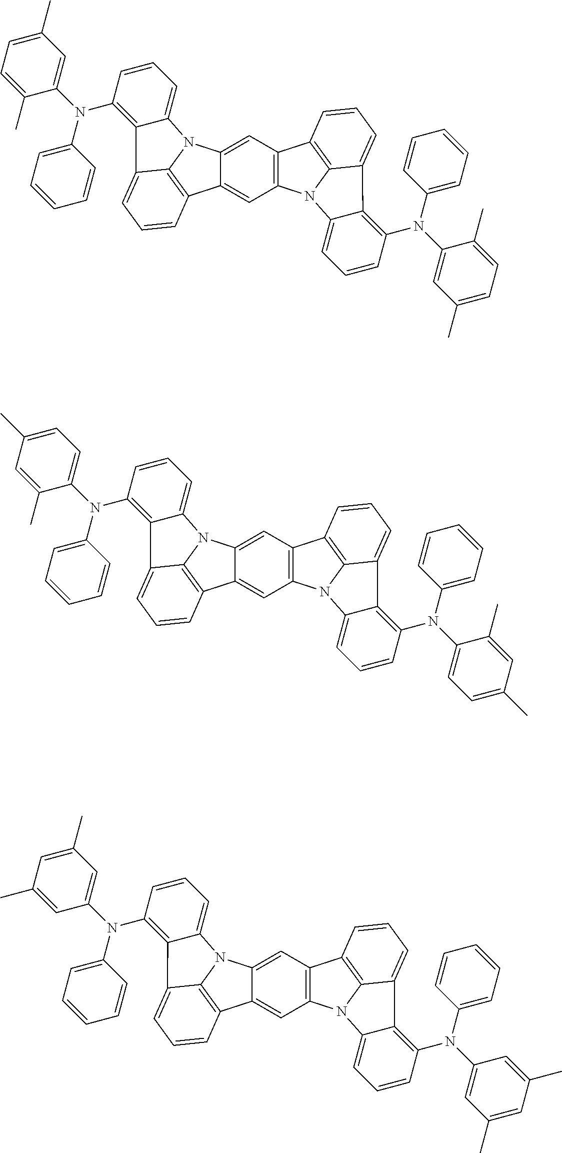

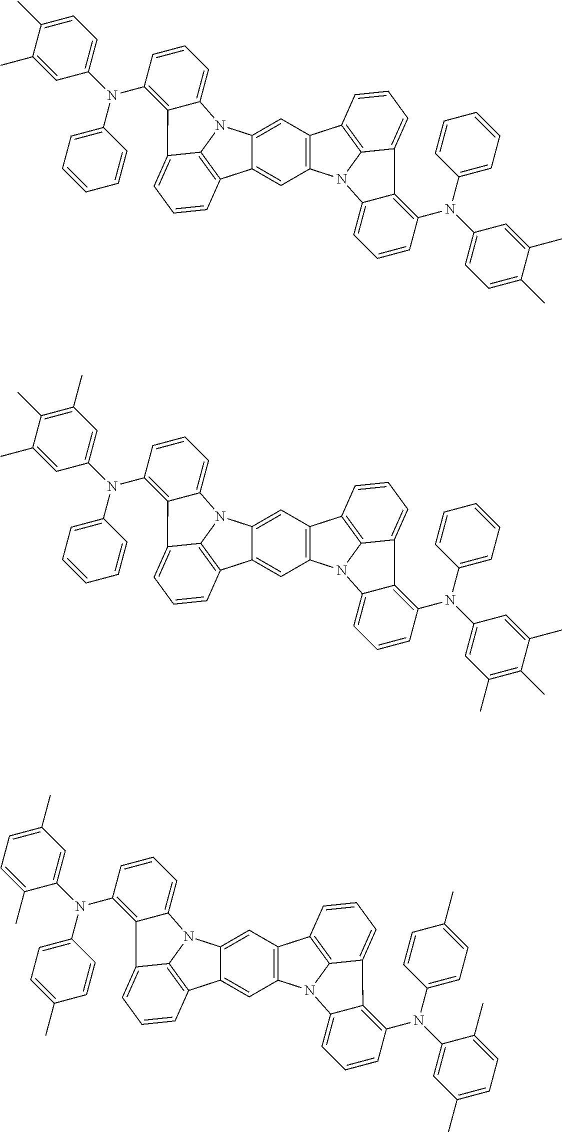

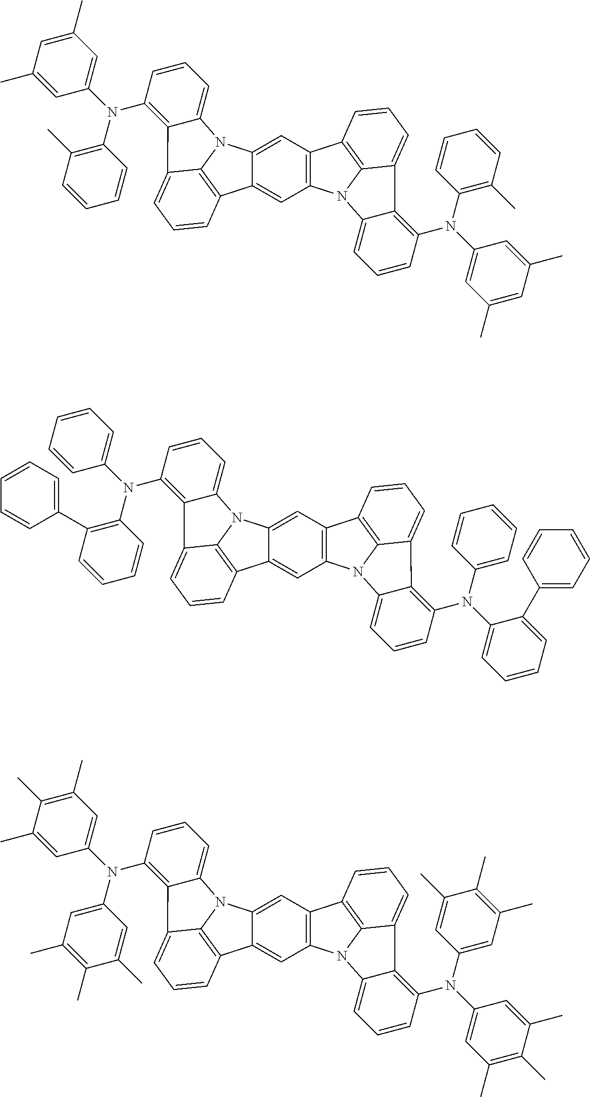

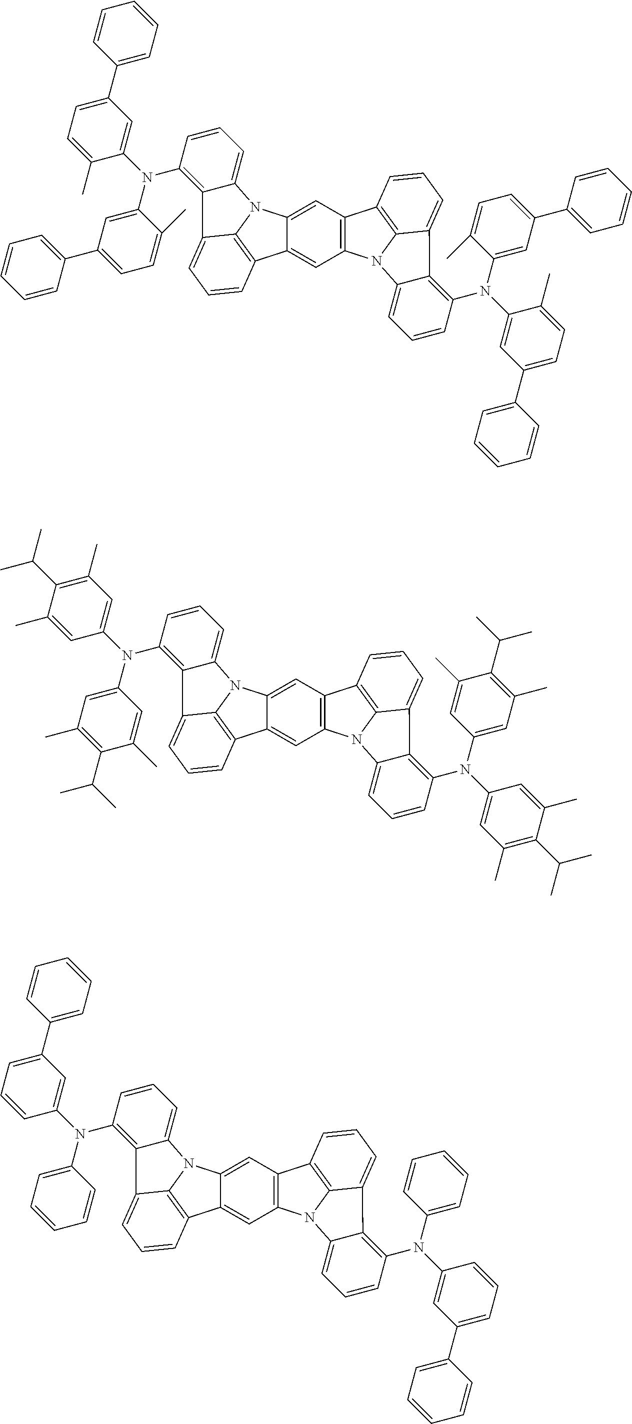 [SMILES: Cc1ccc(-c2ccccc2)cc1N(c1cc(-c2ccccc2)ccc1C)c1cccc2c1c1cccc3c4cc5c(cc4n2c31)c1cccc2c3c(N(c4cc(-c6ccccc6)ccc4C)c4cc(-c6ccccc6)ccc4C)cccc3n5c12 ; Cc1cc(N(c2cc(C)c(C(C)C)c(C)c2)c2cccc3c2c2cccc4c5cc6c(cc5n3c42)c2cccc3c4c(N(c5cc(C)c(C(C)C)c(C)c5)c5cc(C)c(C(C)C)c(C)c5)cccc4n6c23)cc(C)c1C(C)C ; c1ccc(-c2cccc(N(c3ccccc3)c3cccc4c3c3cccc5c6cc7c(cc6n4c53)c3cccc4c5c(N(c6ccccc6)c6cccc(-c8ccccc8)c6)cccc5n7c34)c2)cc1]

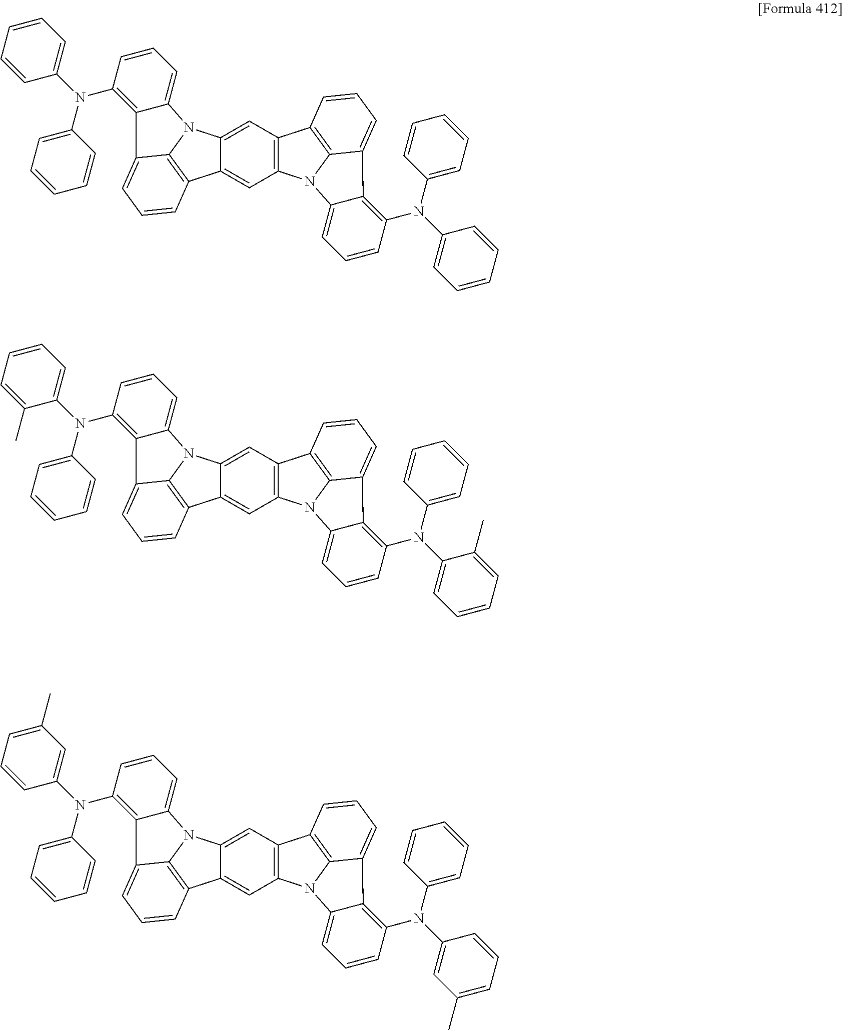 [SMILES: c1ccc(N(c2ccccc2)c2cccc3c2c2cccc4c5cc6c(cc5n3c42)c2cccc3c4c(N(c5ccccc5)c5ccccc5)cccc4n6c23)cc1 ; Cc1ccccc1N(c1ccccc1)c1cccc2c1c1cccc3c4cc5c(cc4n2c31)c1cccc2c3c(N(c4ccccc4)c4ccccc4C)cccc3n5c12 ; Cc1cccc(N(c2ccccc2)c2cccc3c2c2cccc4c5cc6c(cc5n3c42)c2cccc3c4c(N(c5ccccc5)c5cccc(C)c5)cccc4n6c23)c1]

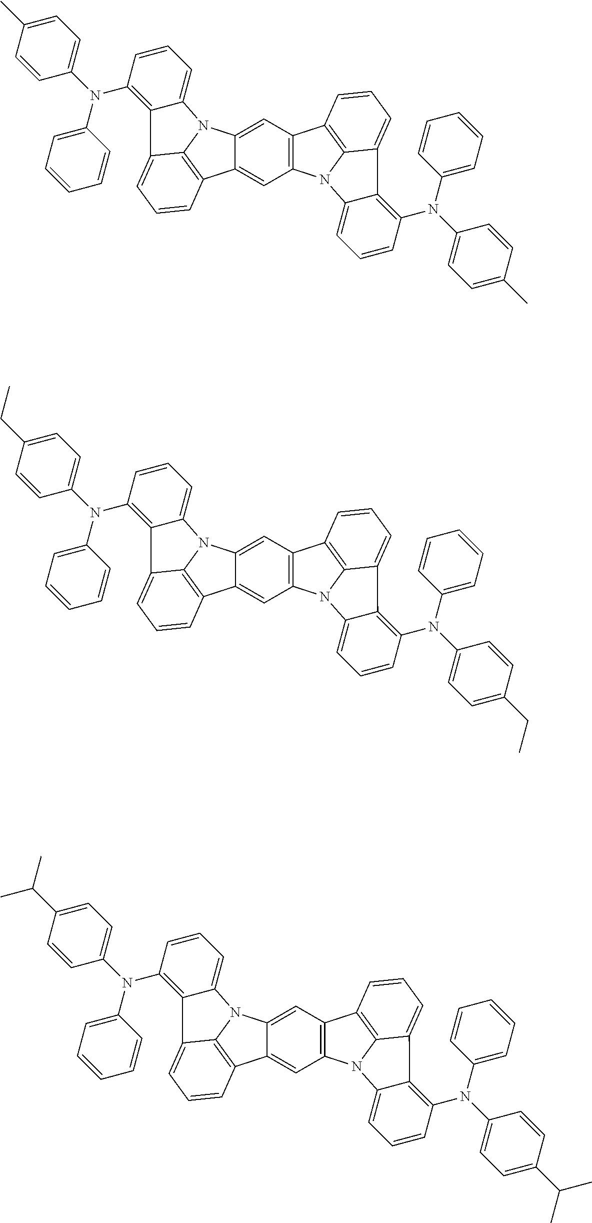

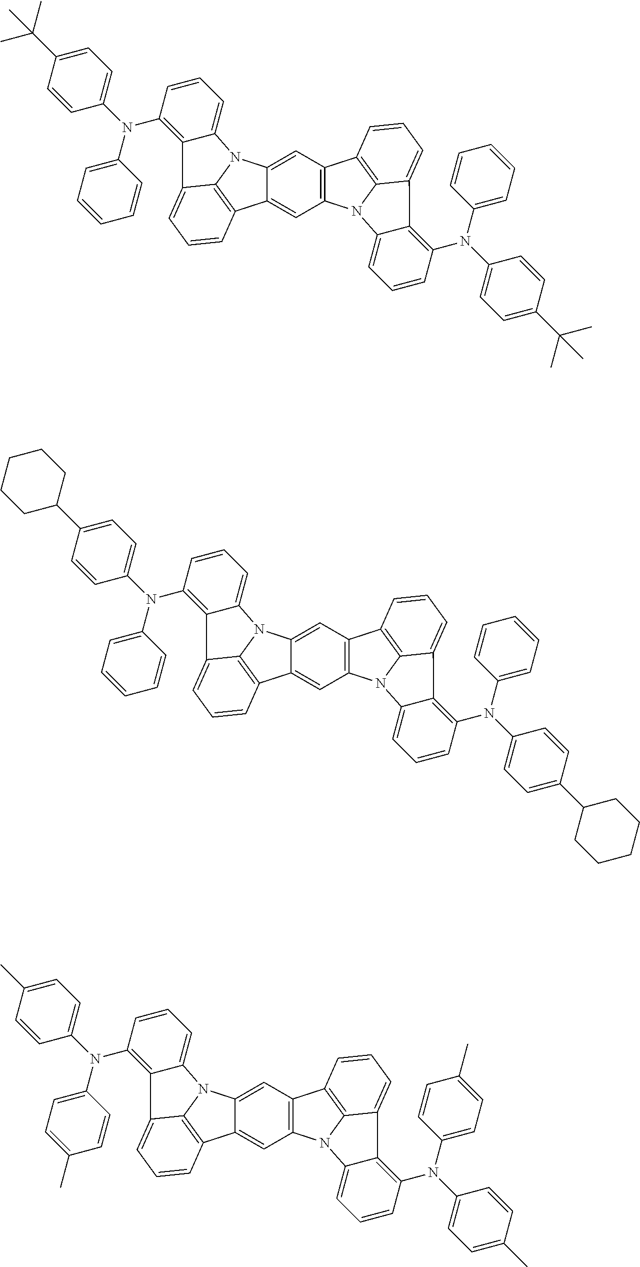

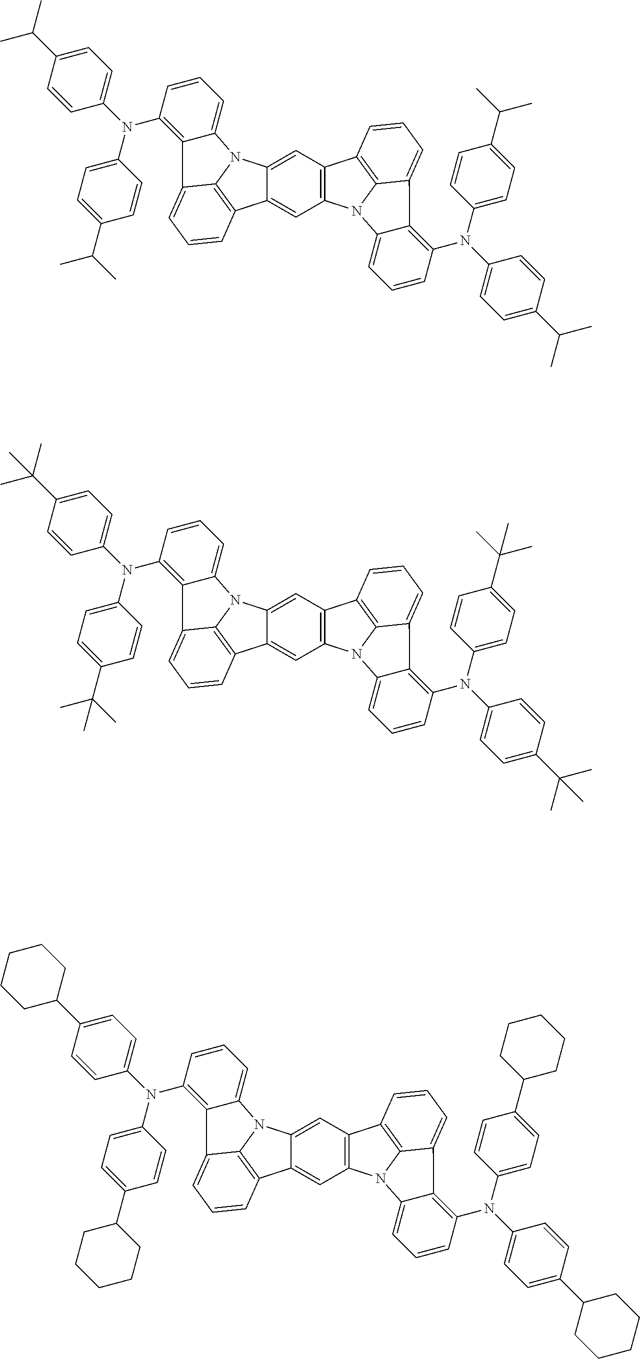

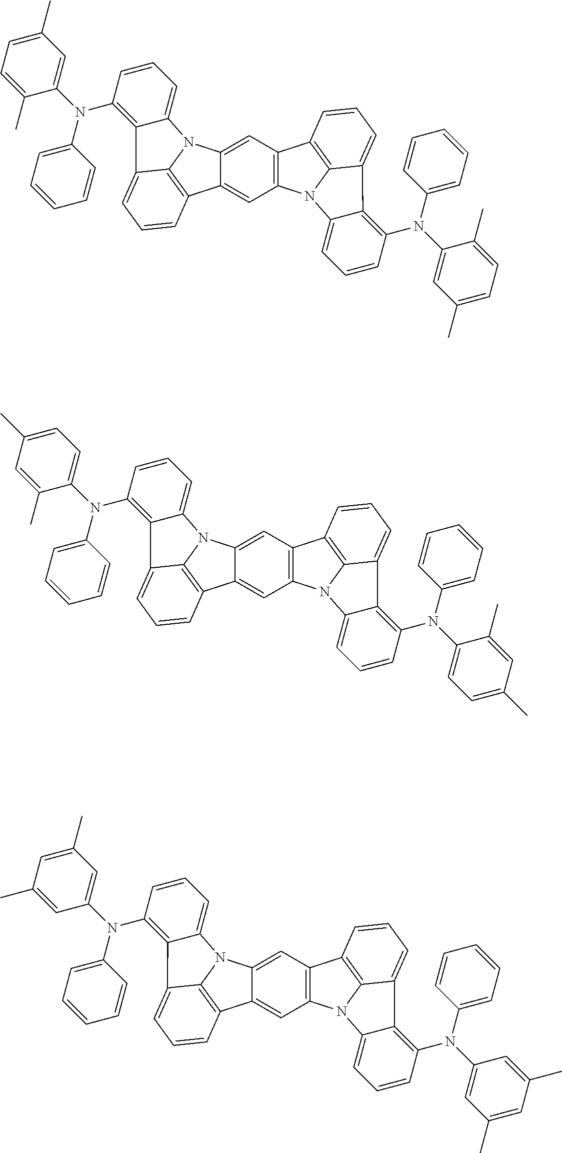

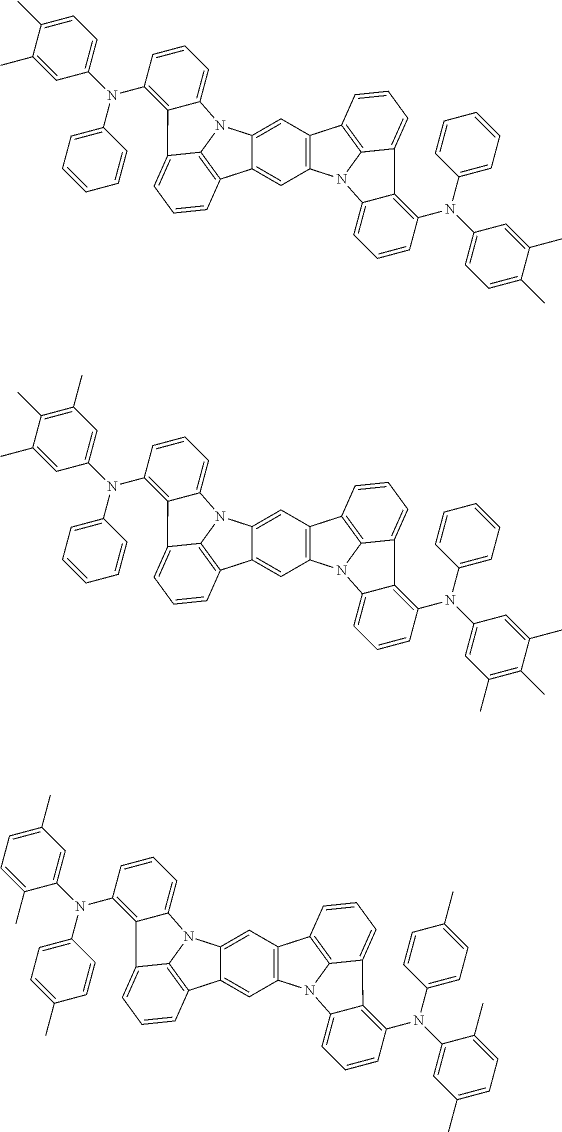

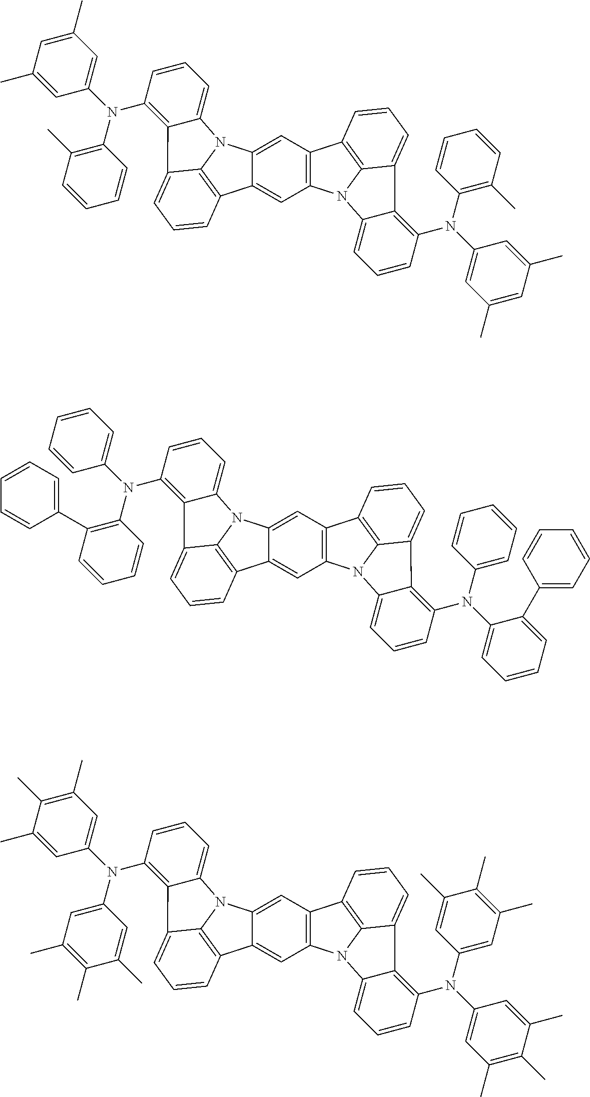

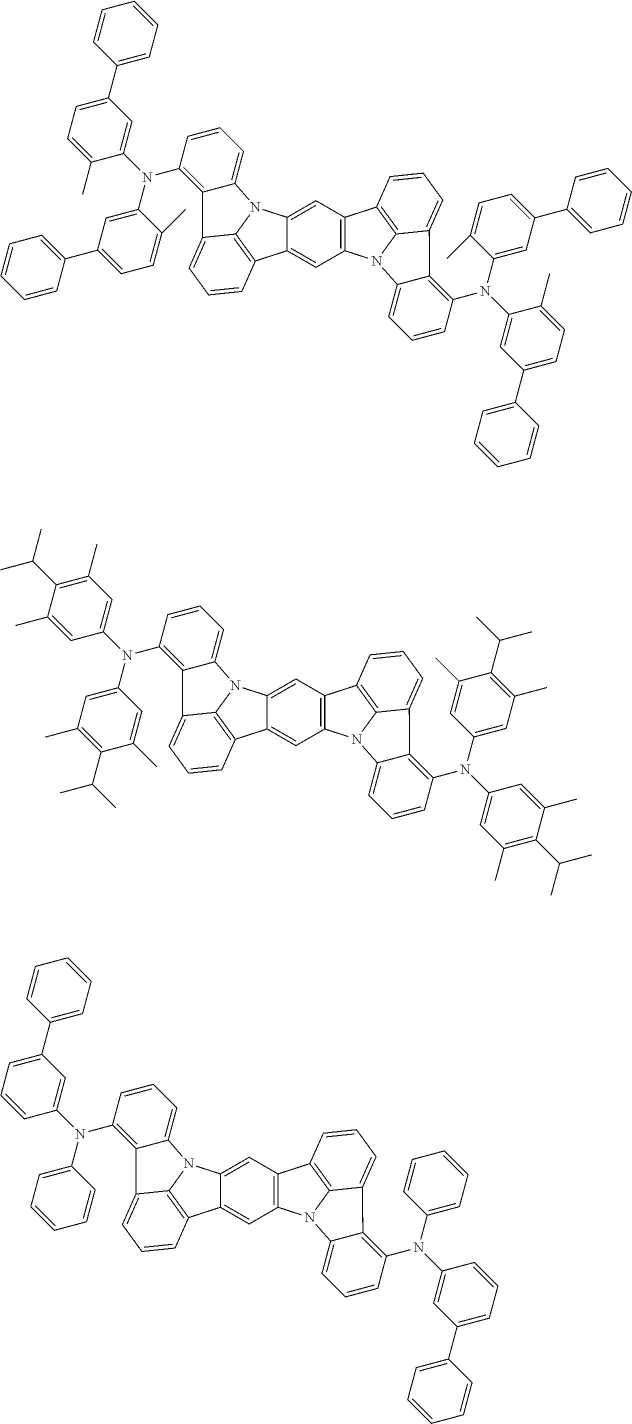 [SMILES: Cc1ccc(-c2ccccc2)cc1N(c1cc(-c2ccccc2)ccc1C)c1cccc2c1c1cccc3c4cc5c(cc4n2c31)c1cccc2c3c(N(c4cc(-c6ccccc6)ccc4C)c4cc(-c6ccccc6)ccc4C)cccc3n5c12 ; Cc1cc(N(c2cc(C)c(C(C)C)c(C)c2)c2cccc3c2c2cccc4c5cc6c(cc5n3c42)c2cccc3c4c(N(c5cc(C)c(C(C)C)c(C)c5)c5cc(C)c(C(C)C)c(C)c5)cccc4n6c23)cc(C)c1C(C)C ; c1ccc(-c2cccc(N(c3ccccc3)c3cccc4c3c3cccc5c6cc7c(cc6n4c53)c3cccc4c5c(N(c6ccccc6)c6cccc(-c8ccccc8)c6)cccc5n7c34)c2)cc1]

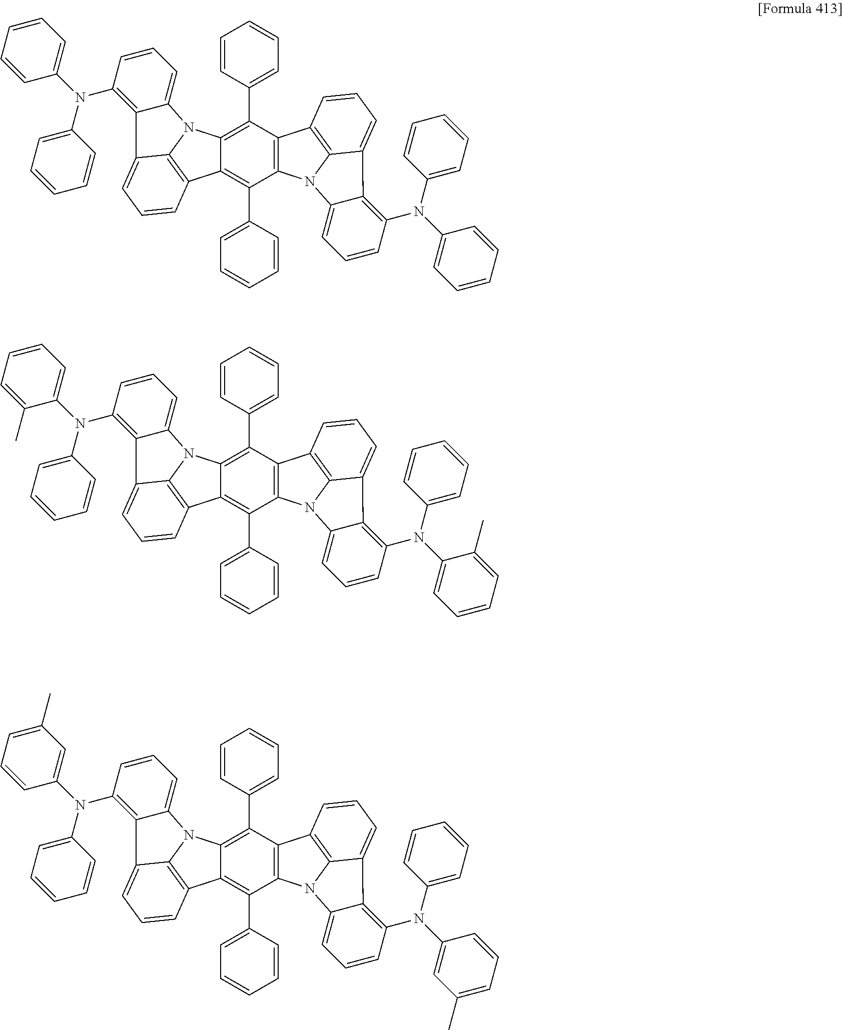 [SMILES: c1ccc(-c2c3c4cccc5c6c(N(c7ccccc7)c7ccccc7)cccc6n(c3c(-c3ccccc3)c3c6cccc7c8c(N(c9ccccc9)c9ccccc9)cccc8n(c23)c76)c54)cc1 ; Cc1ccccc1N(c1ccccc1)c1cccc2c1c1cccc3c4c(-c5ccccc5)c5c(c(-c6ccccc6)c4n2c13)c1cccc2c3c(N(c4ccccc4)c4ccccc4C)cccc3n5c21 ; Cc1cccc(N(c2ccccc2)c2cccc3c2c2cccc4c5c(-c6ccccc6)c6c(c(-c7ccccc7)c5n3c24)c2cccc3c4c(N(c5ccccc5)c5cccc(C)c5)cccc4n6c32)c1]

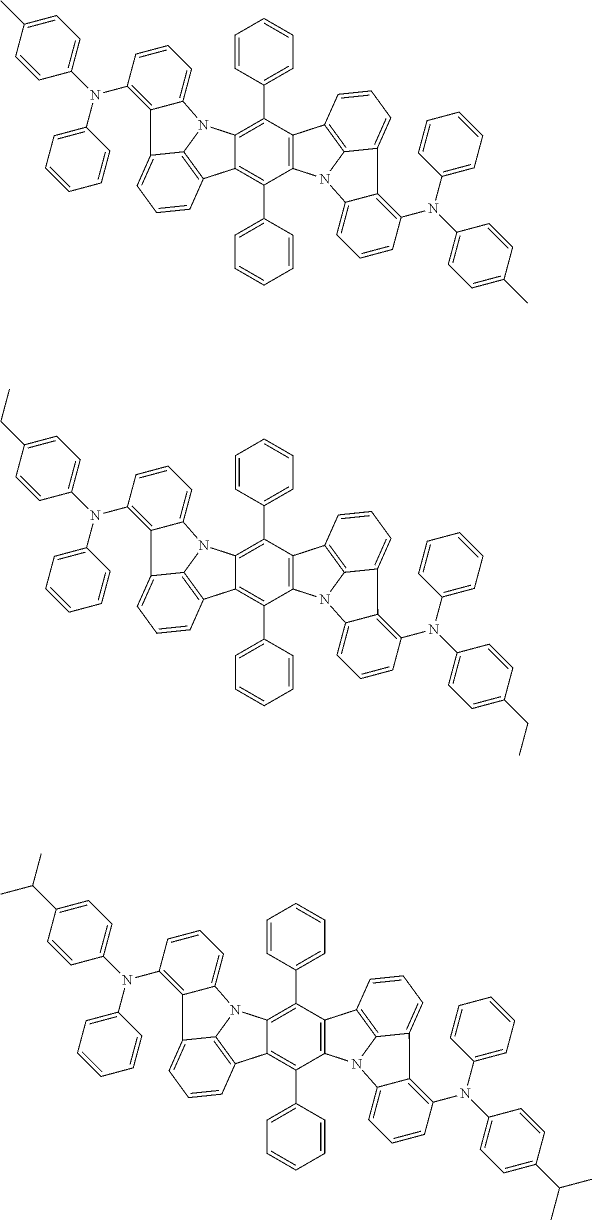

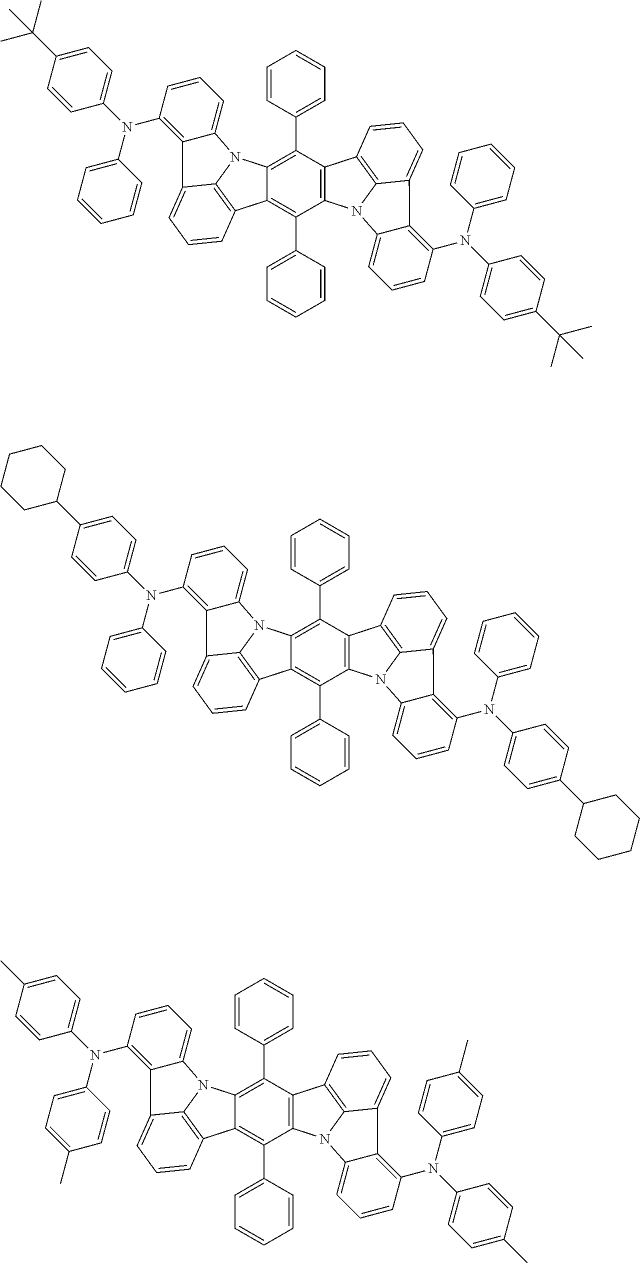

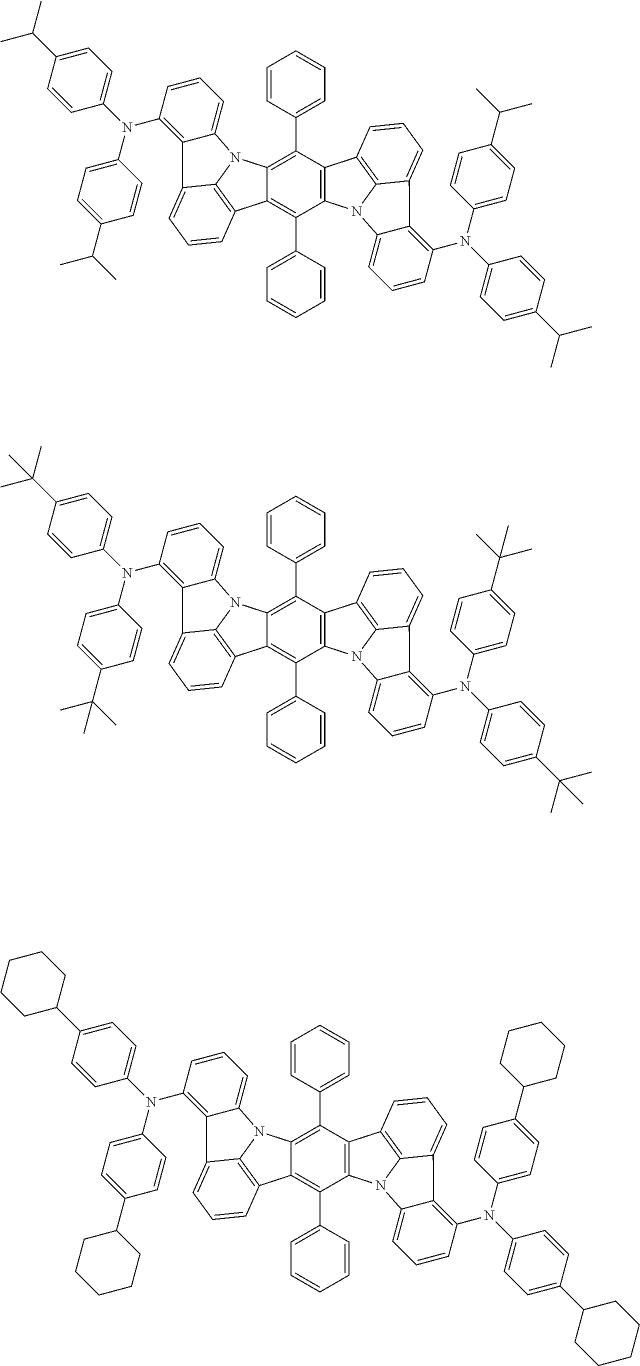

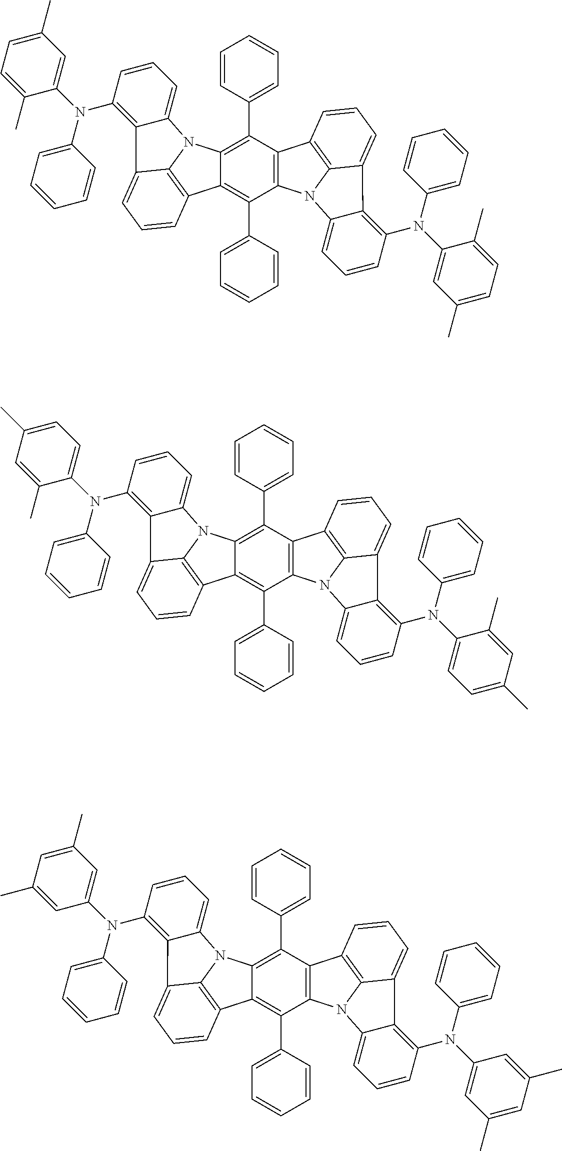

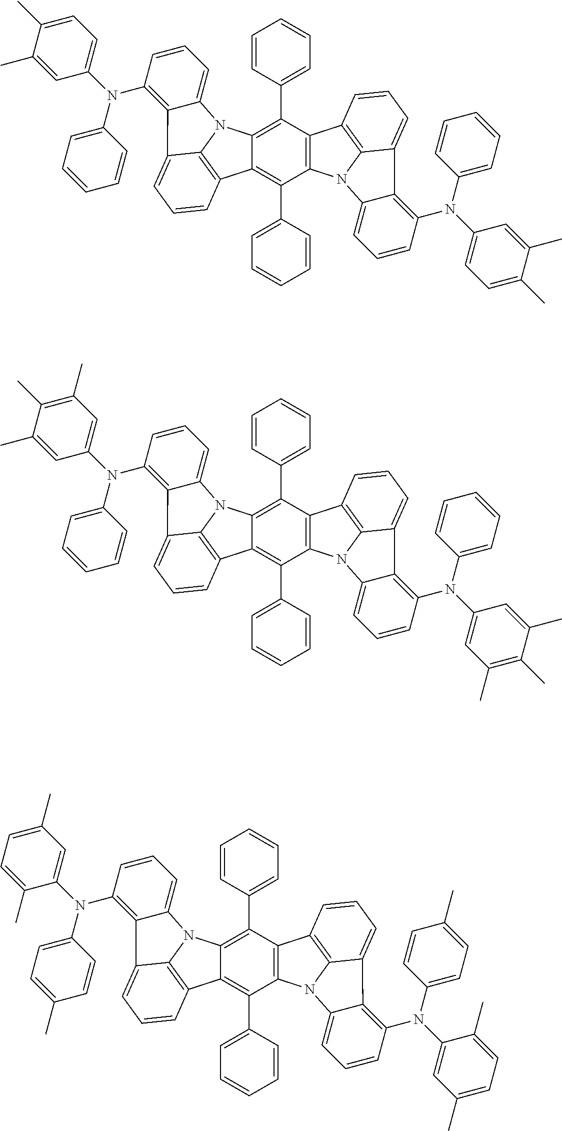

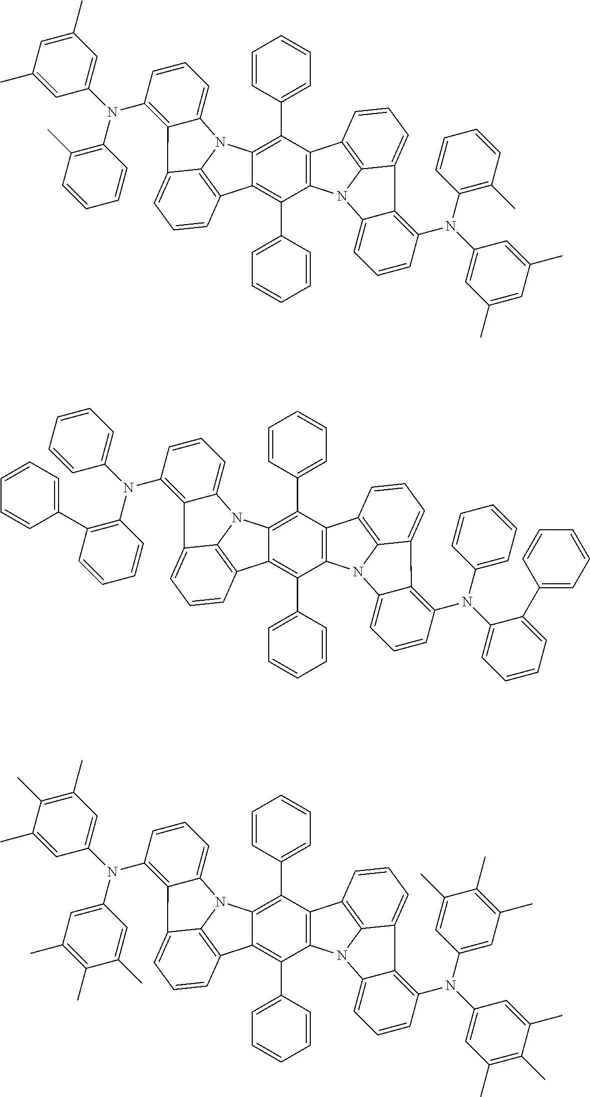

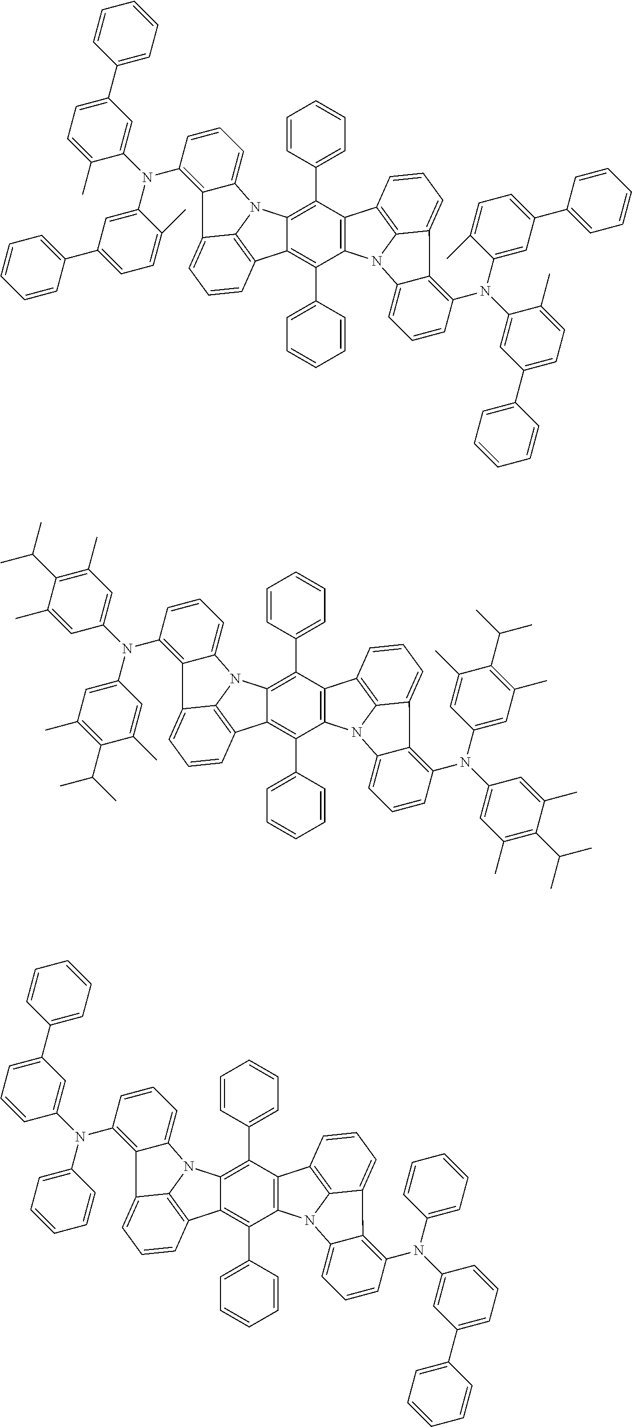 [SMILES: Cc1ccc(-c2ccccc2)cc1N(c1cc(-c2ccccc2)ccc1C)c1cccc2c1c1cccc3c4c(-c5ccccc5)c5c(c(-c6ccccc6)c4n2c13)c1cccc2c3c(N(c4cc(-c6ccccc6)ccc4C)c4cc(-c6ccccc6)ccc4C)cccc3n5c21 ; Cc1cc(N(c2cc(C)c(C(C)C)c(C)c2)c2cccc3c2c2cccc4c5c(-c6ccccc6)c6c(c(-c7ccccc7)c5n3c24)c2cccc3c4c(N(c5cc(C)c(C(C)C)c(C)c5)c5cc(C)c(C(C)C)c(C)c5)cccc4n6c32)cc(C)c1C(C)C ; c1ccc(-c2cccc(N(c3ccccc3)c3cccc4c3c3cccc5c6c(-c7ccccc7)c7c(c(-c8ccccc8)c6n4c35)c3cccc4c5c(N(c6ccccc6)c6cccc(-c8ccccc8)c6)cccc5n7c43)c2)cc1]

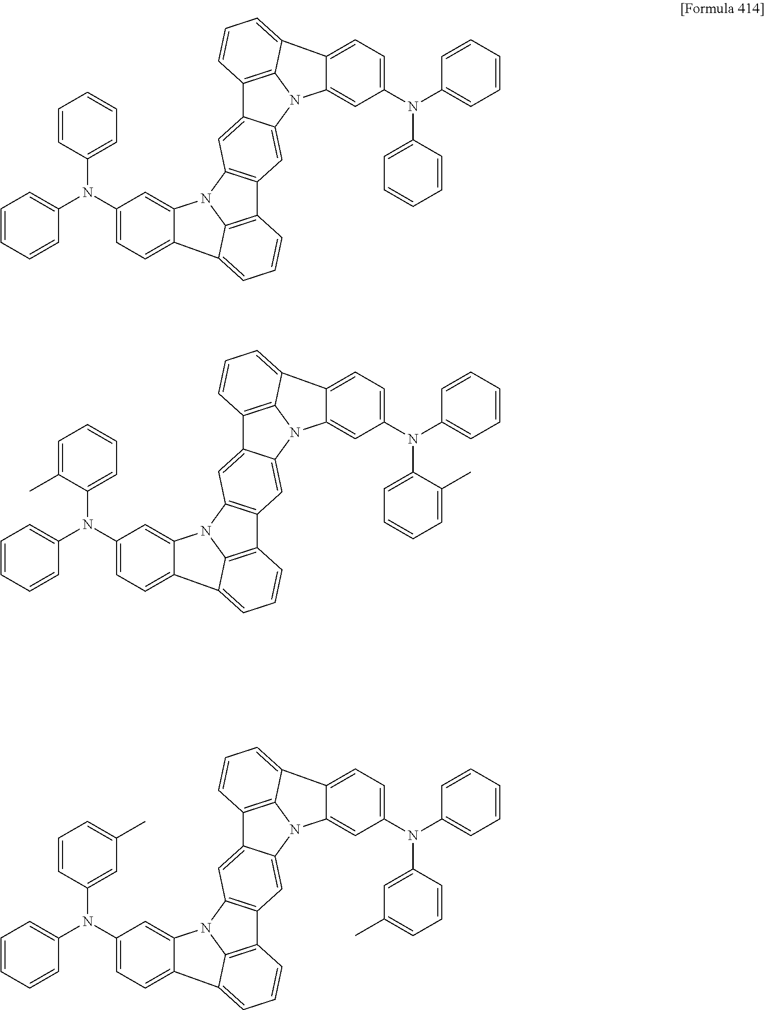 [SMILES: c1ccc(N(c2ccccc2)c2ccc3c4cccc5c6cc7c(cc6n(c3c2)c45)c2cccc3c4ccc(N(c5ccccc5)c5ccccc5)cc4n7c32)cc1 ; Cc1ccccc1N(c1ccccc1)c1ccc2c3cccc4c5cc6c(cc5n(c2c1)c34)c1cccc2c3ccc(N(c4ccccc4)c4ccccc4C)cc3n6c21 ; Cc1cccc(N(c2ccccc2)c2ccc3c4cccc5c6cc7c(cc6n(c3c2)c45)c2cccc3c4ccc(N(c5ccccc5)c5cccc(C)c5)cc4n7c32)c1]

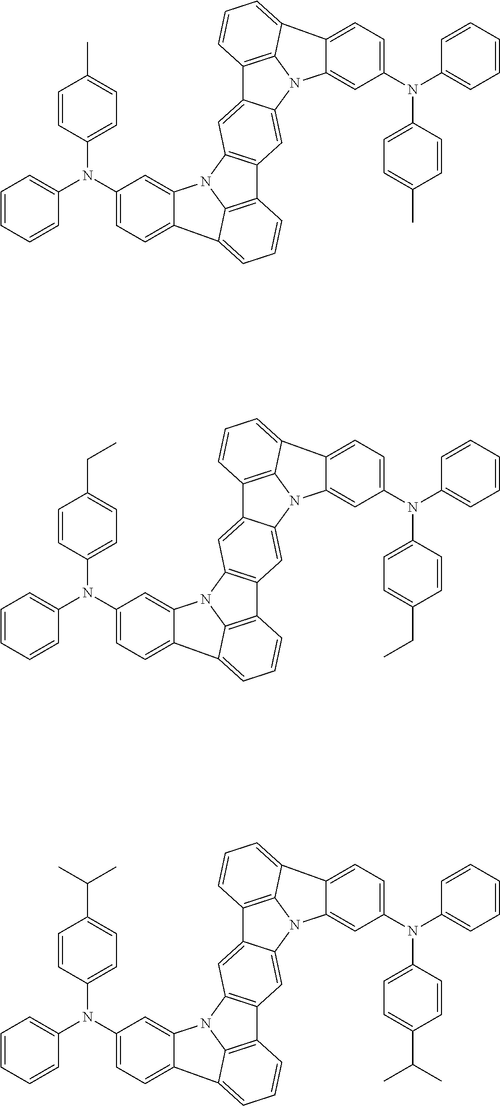 [SMILES: Cc1ccc(N(c2ccccc2)c2ccc3c4cccc5c6cc7c(cc6n(c3c2)c45)c2cccc3c4ccc(N(c5ccccc5)c5ccc(C)cc5)cc4n7c32)cc1 ; CCc1ccc(N(c2ccccc2)c2ccc3c4cccc5c6cc7c(cc6n(c3c2)c45)c2cccc3c4ccc(N(c5ccccc5)c5ccc(CC)cc5)cc4n7c32)cc1 ; CC(C)c1ccc(N(c2ccccc2)c2ccc3c4cccc5c6cc7c(cc6n(c3c2)c45)c2cccc3c4ccc(N(c5ccccc5)c5ccc(C(C)C)cc5)cc4n7c32)cc1]

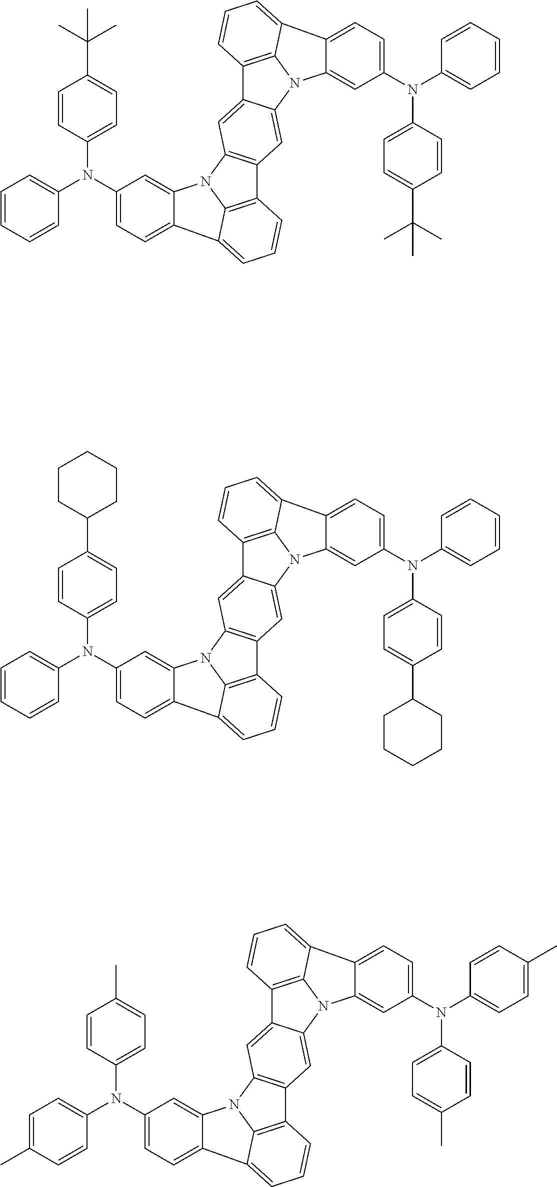

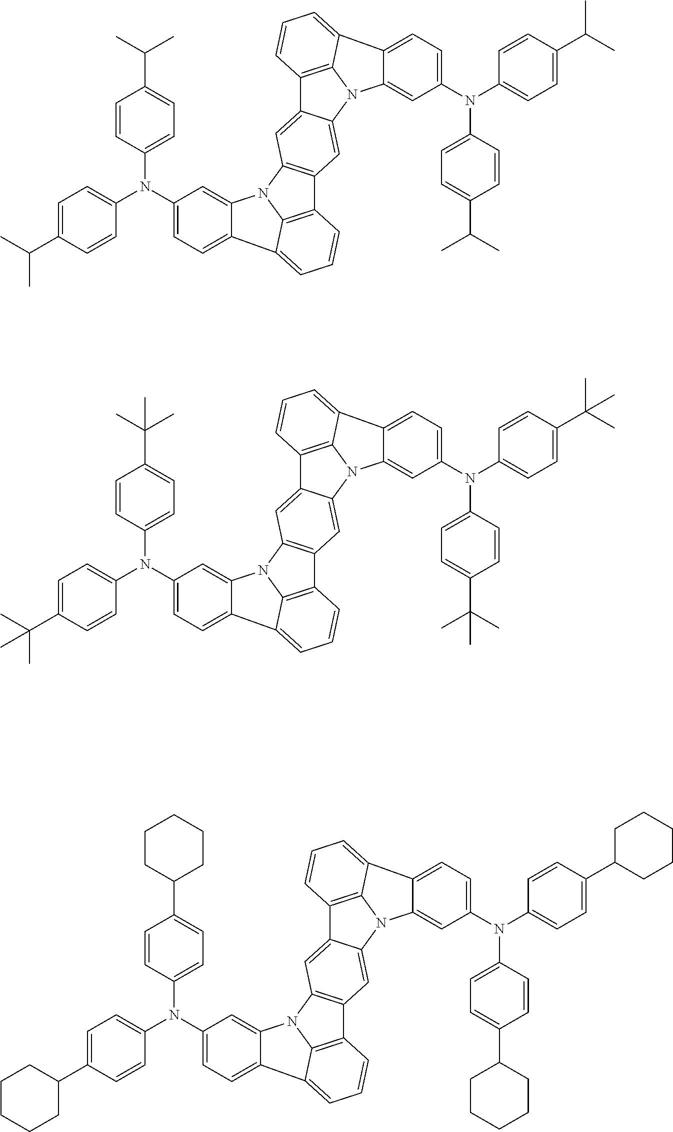

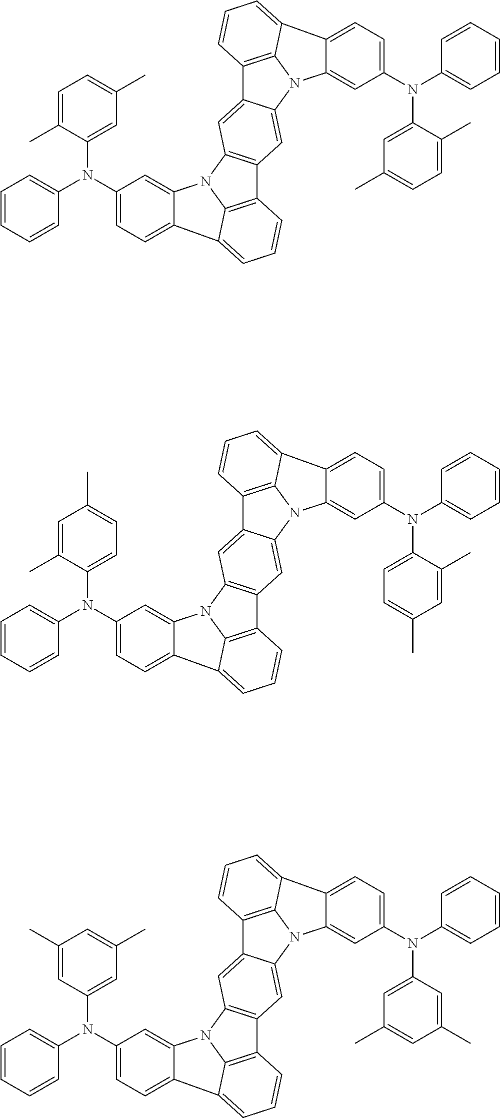 [SMILES: Cc1ccc(C)c(N(c2ccccc2)c2ccc3c4cccc5c6cc7c(cc6n(c3c2)c45)c2cccc3c4ccc(N(c5ccccc5)c5cc(C)ccc5C)cc4n7c32)c1 ; Cc1ccc(N(c2ccccc2)c2ccc3c4cccc5c6cc7c(cc6n(c3c2)c45)c2cccc3c4ccc(N(c5ccccc5)c5ccc(C)cc5C)cc4n7c32)c(C)c1 ; Cc1cc(C)cc(N(c2ccccc2)c2ccc3c4cccc5c6cc7c(cc6n(c3c2)c45)c2cccc3c4ccc(N(c5ccccc5)c5cc(C)cc(C)c5)cc4n7c32)c1]

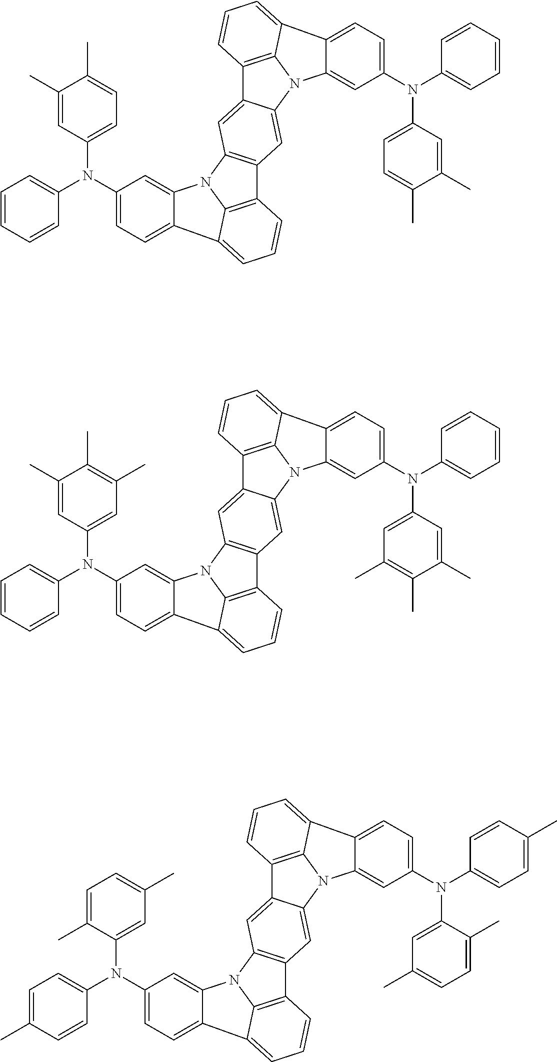

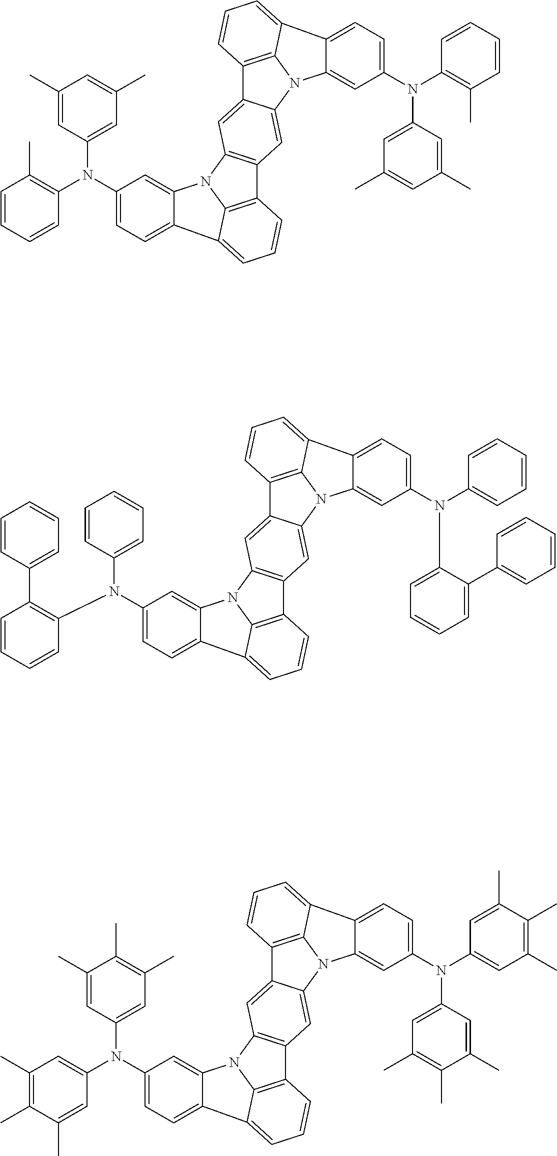

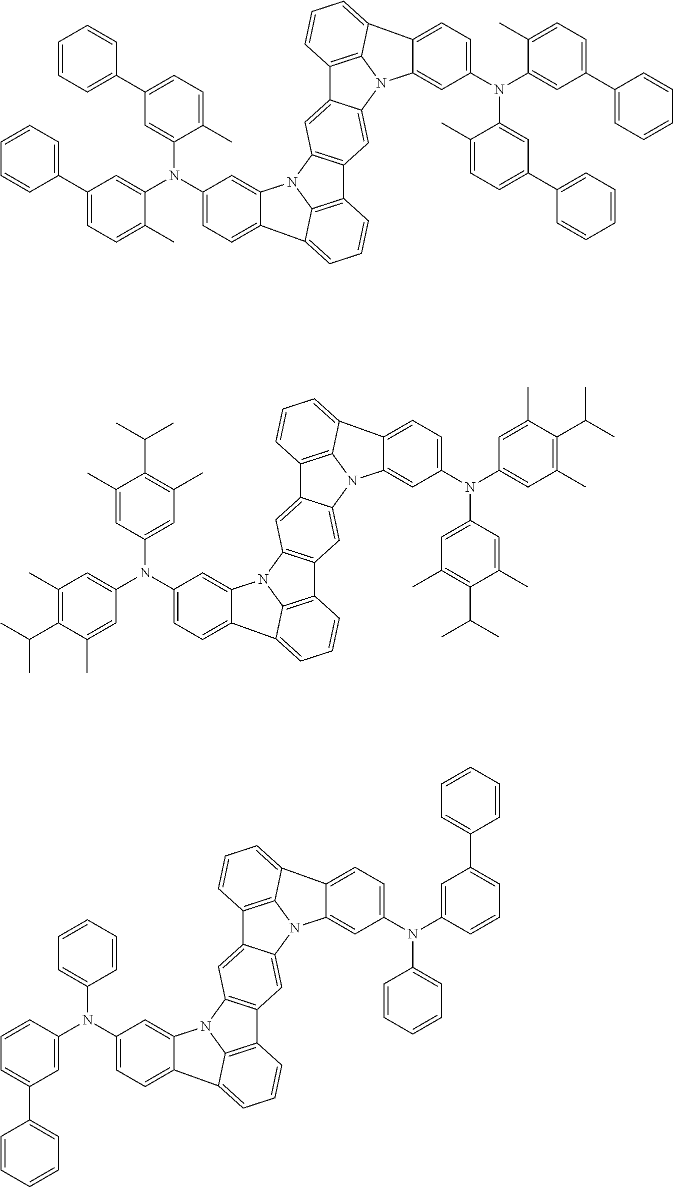 [SMILES: Cc1ccc(-c2ccccc2)cc1N(c1ccc2c3cccc4c5cc6c(cc5n(c2c1)c34)c1cccc2c3ccc(N(c4cc(-c5ccccc5)ccc4C)c4cc(-c5ccccc5)ccc4C)cc3n6c21)c1cc(-c2ccccc2)ccc1C ; Cc1cc(N(c2cc(C)c(C(C)C)c(C)c2)c2ccc3c4cccc5c6cc7c(cc6n(c3c2)c45)c2cccc3c4ccc(N(c5cc(C)c(C(C)C)c(C)c5)c5cc(C)c(C(C)C)c(C)c5)cc4n7c32)cc(C)c1C(C)C ; c1ccc(-c2cccc(N(c3ccccc3)c3ccc4c5cccc6c7cc8c(cc7n(c4c3)c56)c3cccc4c5ccc(N(c6ccccc6)c6cccc(-c7ccccc7)c6)cc5n8c43)c2)cc1]

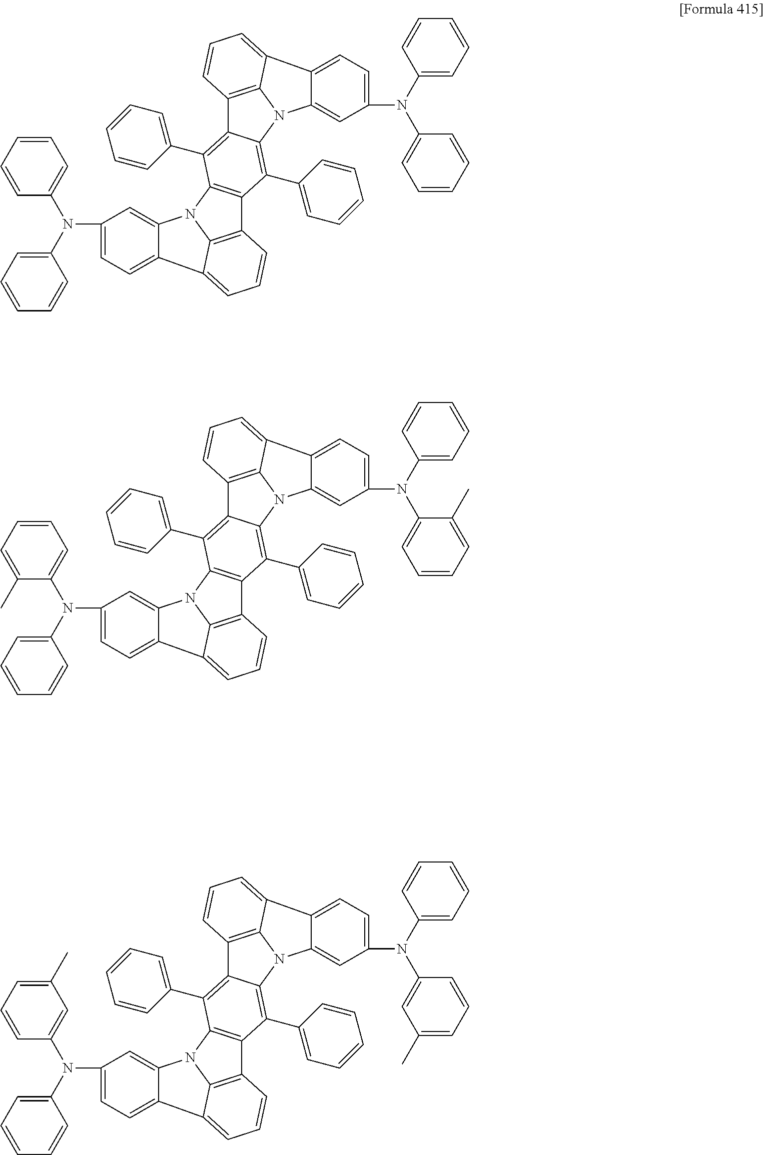 [SMILES: c1ccc(-c2c3c4cccc5c6ccc(N(c7ccccc7)c7ccccc7)cc6n(c3c(-c3ccccc3)c3c6cccc7c8ccc(N(c9ccccc9)c9ccccc9)cc8n(c23)c76)c54)cc1 ; Cc1ccccc1N(c1ccccc1)c1ccc2c3cccc4c5c(-c6ccccc6)c6c(c(-c7ccccc7)c5n(c2c1)c34)c1cccc2c3ccc(N(c4ccccc4)c4ccccc4C)cc3n6c21 ; Cc1cccc(N(c2ccccc2)c2ccc3c4cccc5c6c(-c7ccccc7)c7c(c(-c8ccccc8)c6n(c3c2)c45)c2cccc3c4ccc(N(c5ccccc5)c5cccc(C)c5)cc4n7c32)c1]

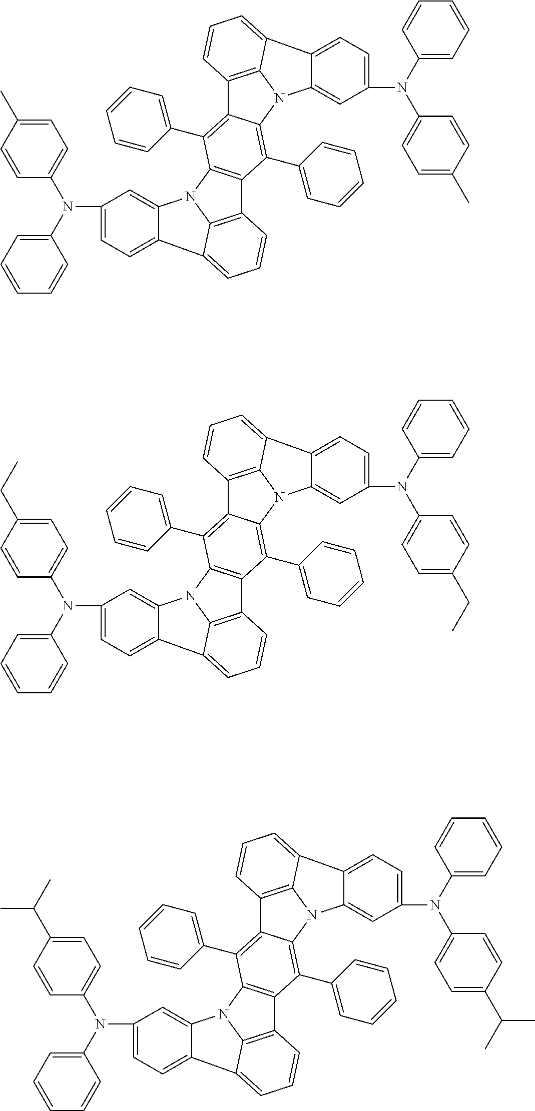

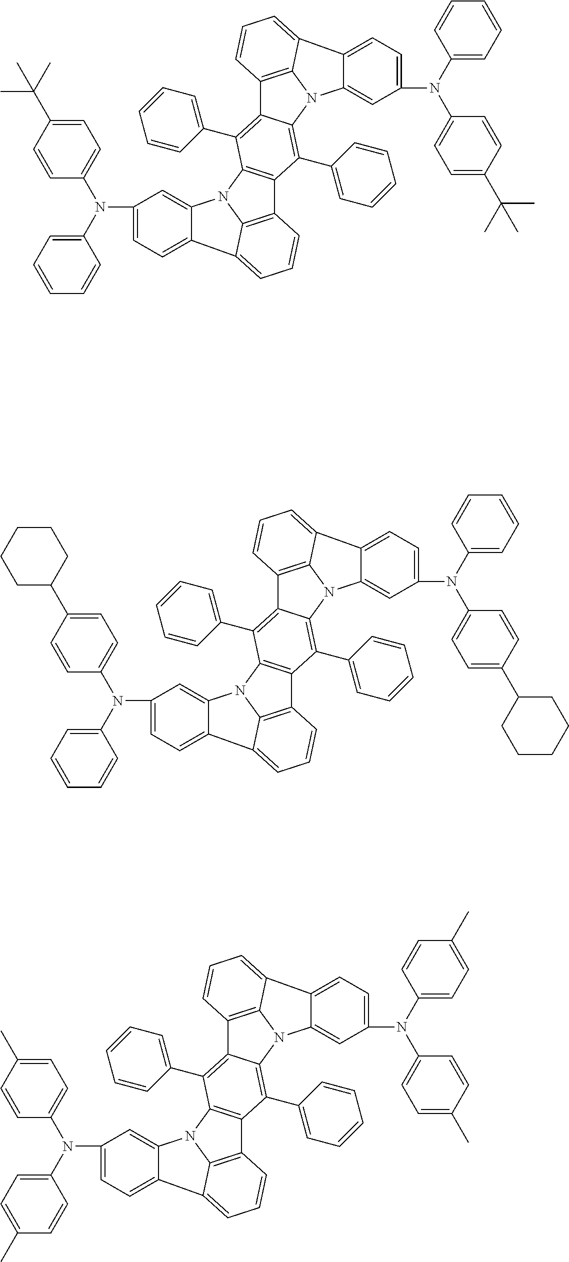 [SMILES: CC(C)(C)c1ccc(N(c2ccccc2)c2ccc3c4cccc5c6c(-c7ccccc7)c7c(c(-c8ccccc8)c6n(c3c2)c45)c2cccc3c4ccc(N(c5ccccc5)c5ccc(C(C)(C)C)cc5)cc4n7c32)cc1 ; c1ccc(-c2c3c4cccc5c6ccc(N(c7ccccc7)c7ccc(C8CCCCC8)cc7)cc6n(c3c(-c3ccccc3)c3c6cccc7c8ccc(N(c9ccccc9)c9ccc(C%10CCCCC%10)cc9)cc8n(c23)c76)c54)cc1 ; Cc1ccc(N(c2ccc(C)cc2)c2ccc3c4cccc5c6c(-c7ccccc7)c7c(c(-c8ccccc8)c6n(c3c2)c45)c2cccc3c4ccc(N(c5ccc(C)cc5)c5ccc(C)cc5)cc4n7c32)cc1]

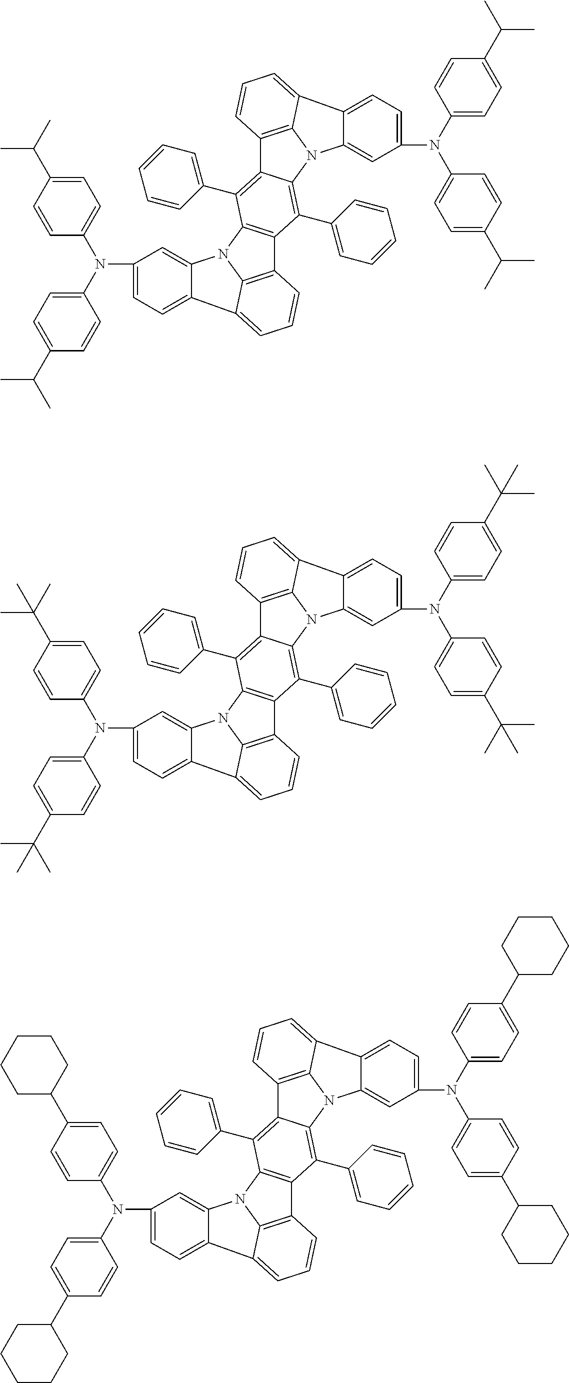

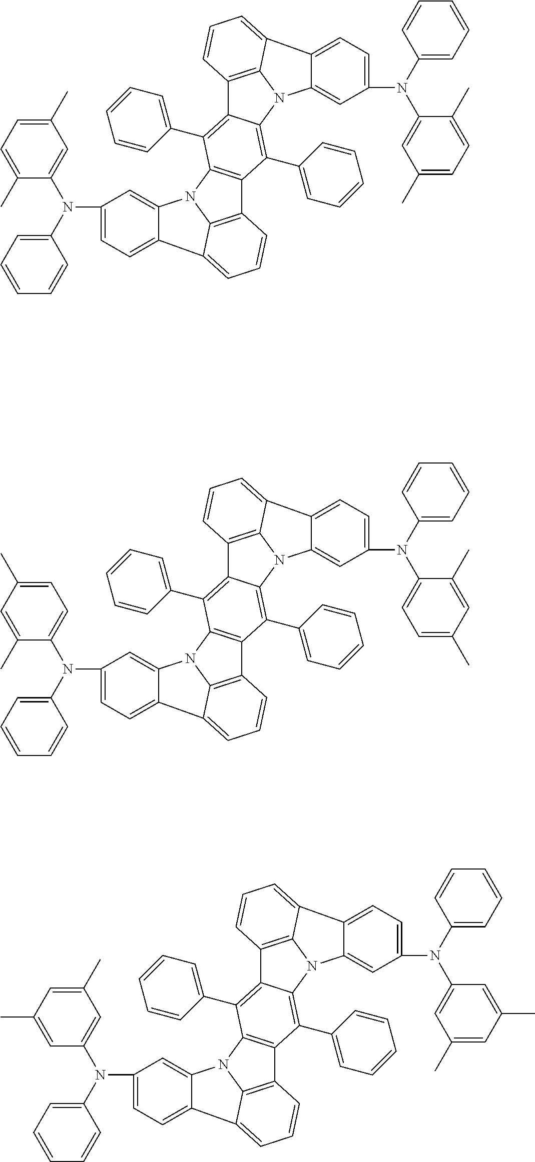

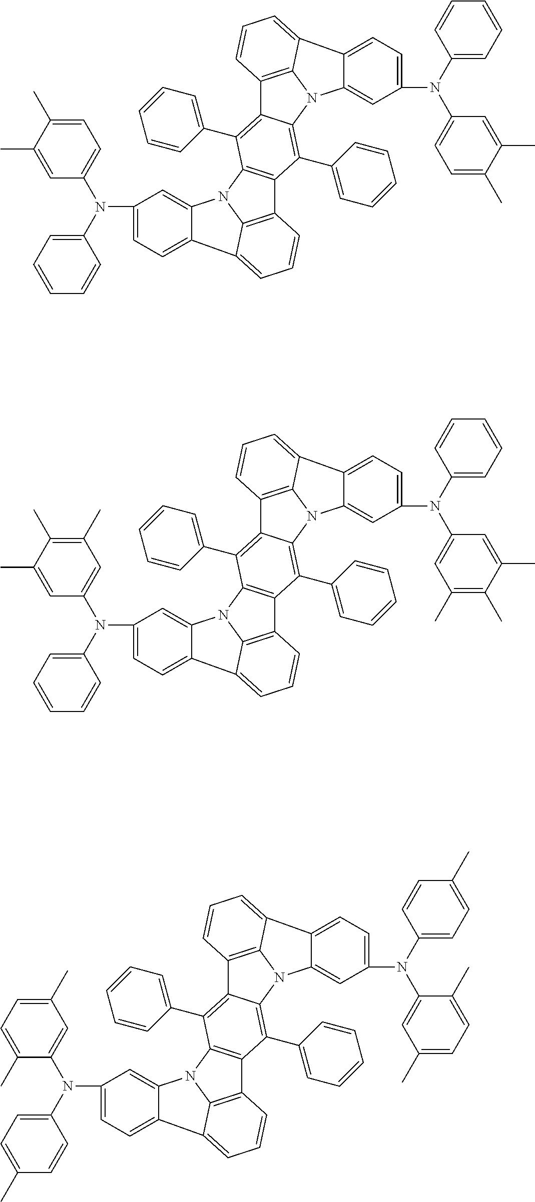

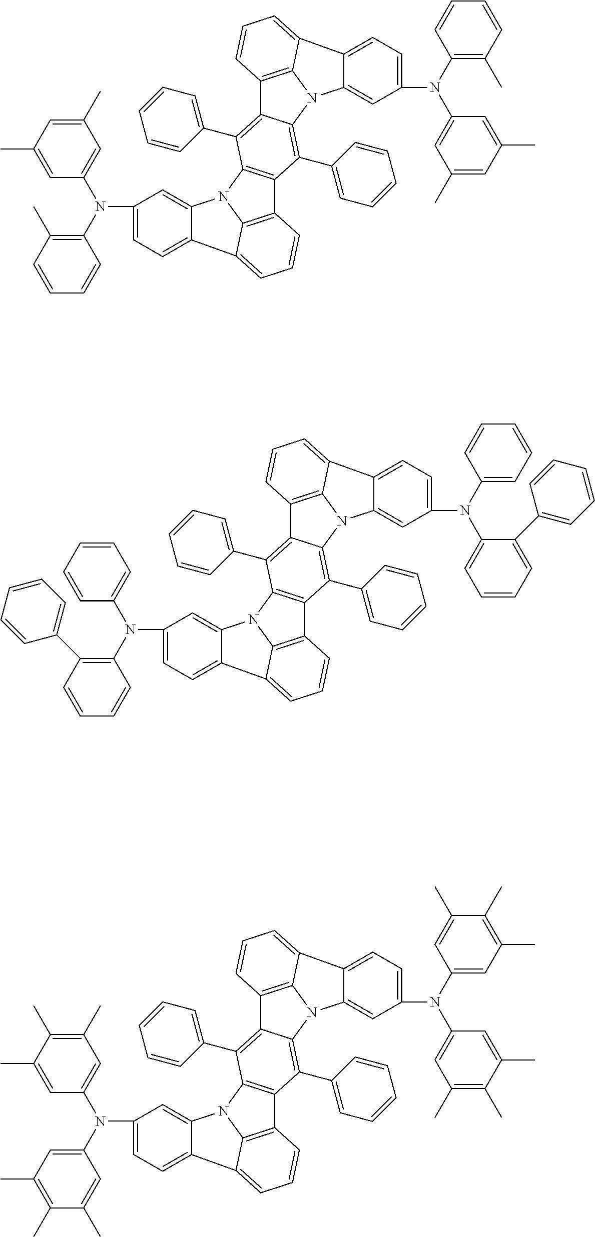

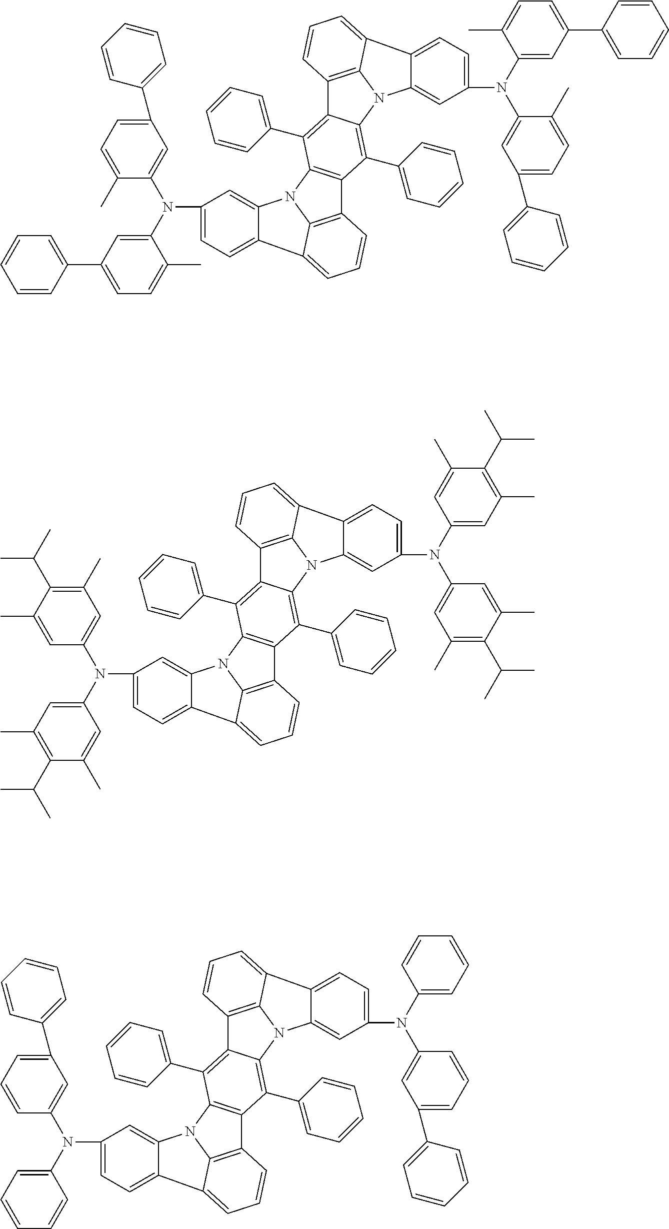 [SMILES: Cc1ccc(-c2ccccc2)cc1N(c1ccc2c3cccc4c5c(-c6ccccc6)c6c(c(-c7ccccc7)c5n(c2c1)c34)c1cccc2c3ccc(N(c4cc(-c5ccccc5)ccc4C)c4cc(-c5ccccc5)ccc4C)cc3n6c21)c1cc(-c2ccccc2)ccc1C ; Cc1cc(N(c2cc(C)c(C(C)C)c(C)c2)c2ccc3c4cccc5c6c(-c7ccccc7)c7c(c(-c8ccccc8)c6n(c3c2)c45)c2cccc3c4ccc(N(c5cc(C)c(C(C)C)c(C)c5)c5cc(C)c(C(C)C)c(C)c5)cc4n7c32)cc(C)c1C(C)C ; c1ccc(-c2cccc(N(c3ccccc3)c3ccc4c5cccc6c7c(-c8ccccc8)c8c(c(-c9ccccc9)c7n(c4c3)c56)c3cccc4c5ccc(N(c6ccccc6)c6cccc(-c7ccccc7)c6)cc5n8c43)c2)cc1]

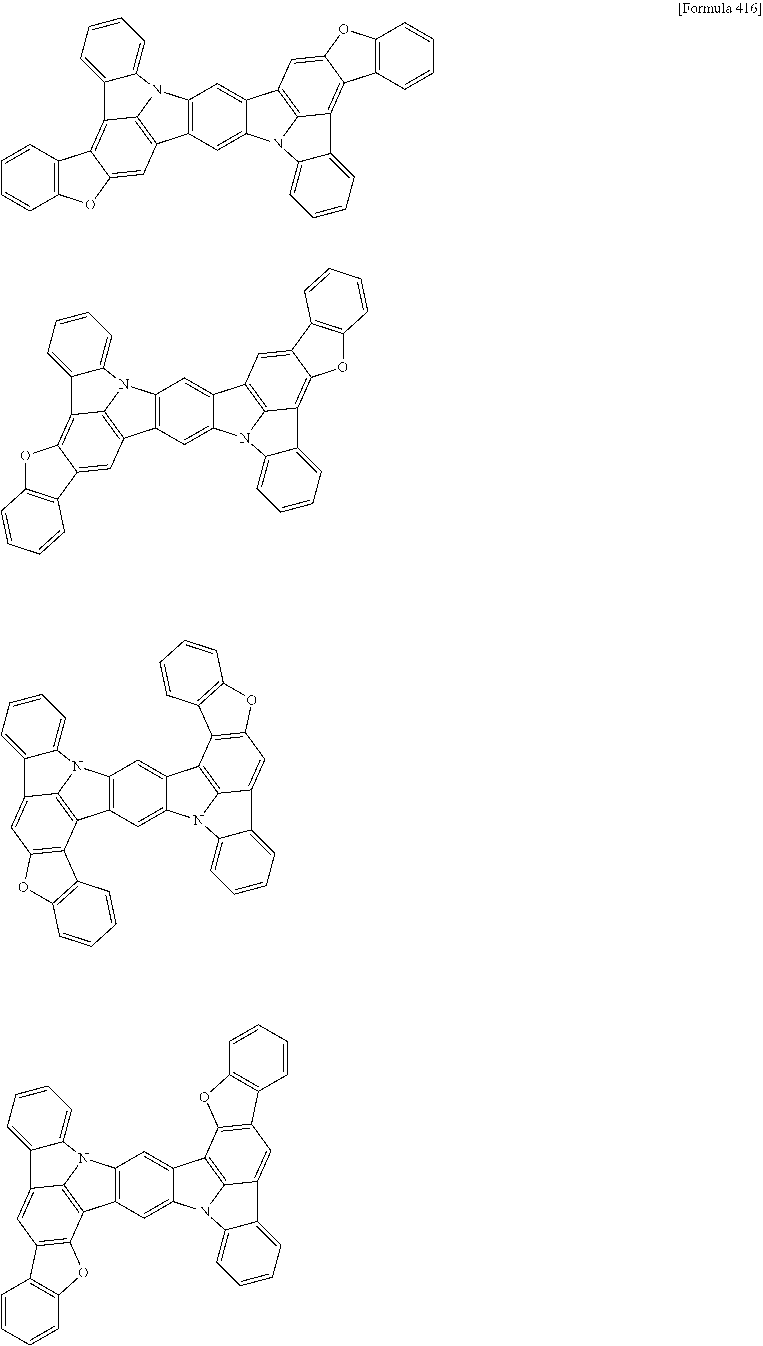 [SMILES: c1ccc2c(c1)oc1cc3c4cc5c(cc4n4c6ccccc6c(c12)c34)c1cc2oc3ccccc3c2c2c3ccccc3n5c12 ; c1ccc2c(c1)oc1c2cc2c3cc4c(cc3n3c5ccccc5c1c23)c1cc2c3ccccc3oc2c2c3ccccc3n4c12 ; c1ccc2c(c1)oc1cc3c4ccccc4n4c5cc6c7c8c(cc9c%10ccccc%10n(c6cc5c(c12)c34)c97)oc1ccccc18 ; c1ccc2c(c1)oc1c2cc2c3ccccc3n3c4cc5c6c7oc8ccccc8c7cc7c8ccccc8n(c5cc4c1c23)c76]

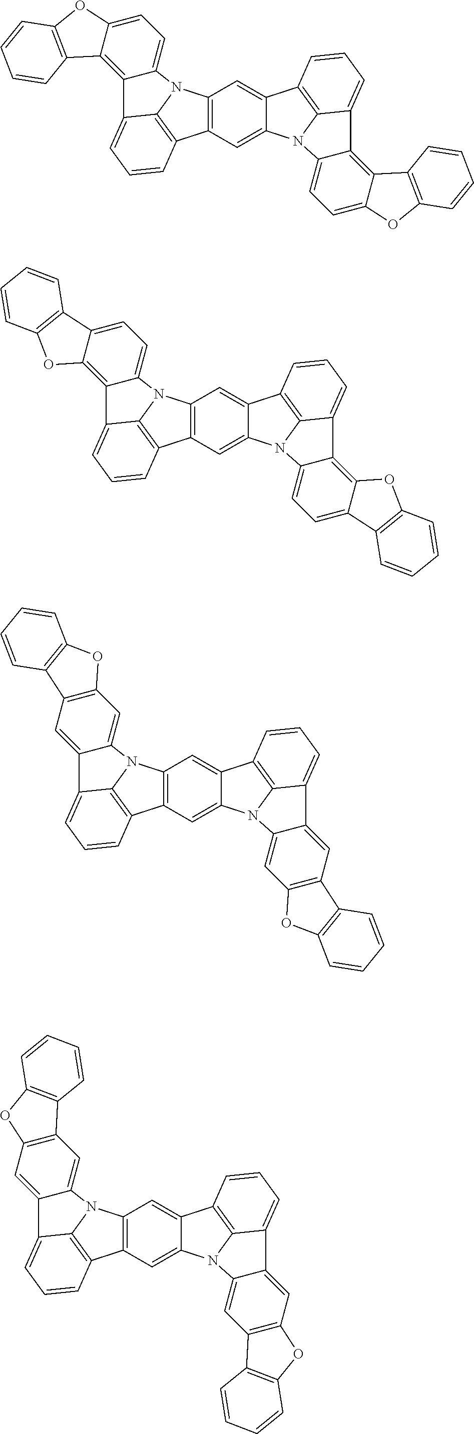 [SMILES: c1ccc2c(c1)oc1ccc3c(c4cccc5c6cc7c(cc6n3c54)c3cccc4c5c6c(ccc5n7c34)oc3ccccc36)c12 ; c1ccc2c(c1)oc1c2ccc2c1c1cccc3c4cc5c(cc4n2c31)c1cccc2c3c4oc6ccccc6c4ccc3n5c12 ; c1ccc2c(c1)oc1cc3c(cc12)c1cccc2c4cc5c(cc4n3c12)c1cccc2c3cc4c(cc3n5c21)oc1ccccc14 ; c1ccc2c(c1)oc1cc3c4cccc5c6cc7c(cc6n(c3cc12)c45)c1cccc2c3cc4oc5ccccc5c4cc3n7c21]

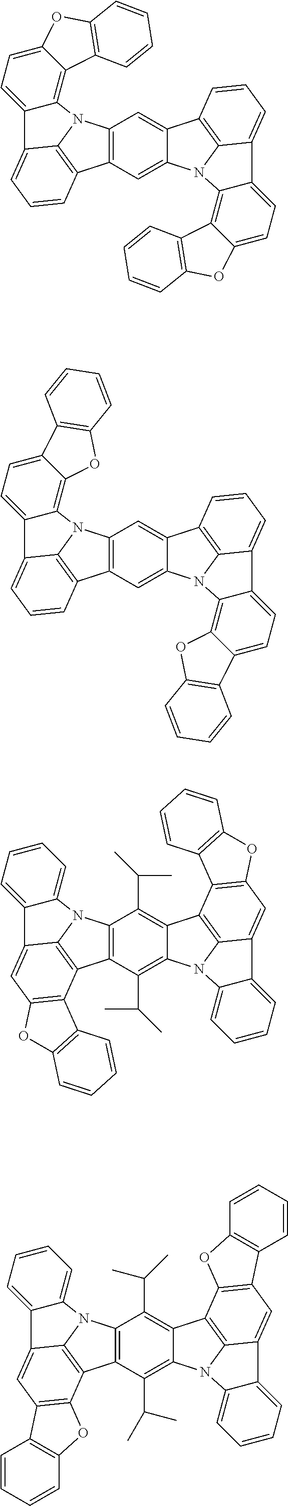 [SMILES: c1ccc2c(c1)oc1ccc3c4cccc5c6cc7c(cc6n(c54)c3c12)c1cccc2c3ccc4oc5ccccc5c4c3n7c12 ; c1ccc2c(c1)oc1c2ccc2c3cccc4c5cc6c(cc5n(c43)c21)c1cccc2c3ccc4c5ccccc5oc4c3n6c12 ; CC(C)c1c2c3c4c(cc5c6ccccc6n(c2c(C(C)C)c2c6c7c(cc8c9ccccc9n(c12)c86)oc1ccccc17)c53)oc1ccccc14 ; CC(C)c1c2c3c4oc5ccccc5c4cc4c5ccccc5n(c2c(C(C)C)c2c5c6oc7ccccc7c6cc6c7ccccc7n(c12)c65)c43]

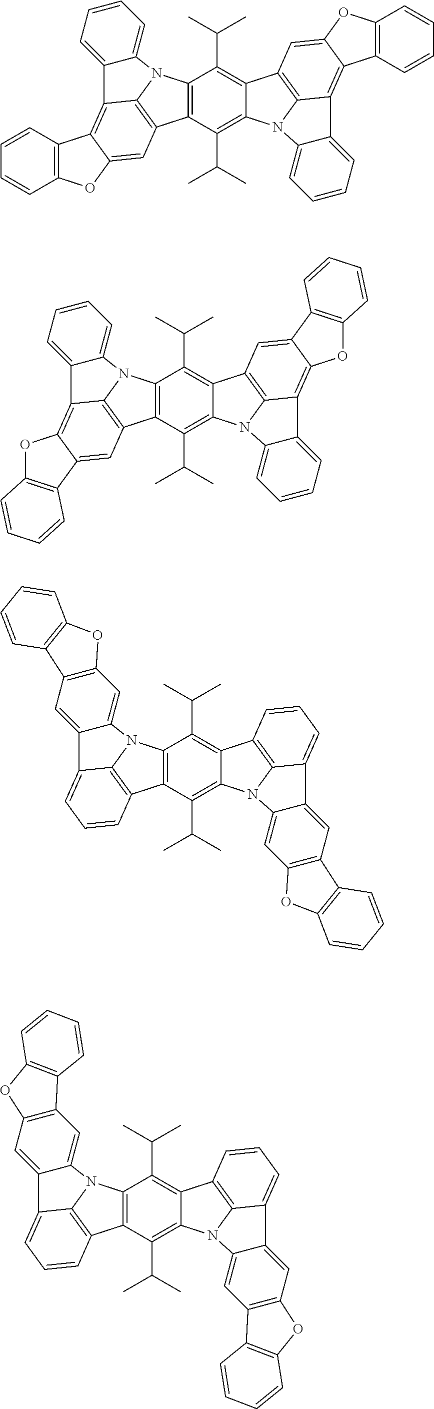 [SMILES: CC(C)c1c2c3cc4oc5ccccc5c4c4c5ccccc5n(c2c(C(C)C)c2c5cc6oc7ccccc7c6c6c7ccccc7n(c12)c56)c34 ; CC(C)c1c2c3cc4c5ccccc5oc4c4c5ccccc5n(c2c(C(C)C)c2c5cc6c7ccccc7oc6c6c7ccccc7n(c12)c56)c34 ; CC(C)c1c2c3cccc4c5cc6c(cc5n(c2c(C(C)C)c2c5cccc7c8cc9c(cc8n(c12)c75)oc1ccccc19)c43)oc1ccccc16 ; CC(C)c1c2c3cccc4c5cc6oc7ccccc7c6cc5n(c2c(C(C)C)c2c5cccc6c7cc8oc9ccccc9c8cc7n(c12)c65)c43]

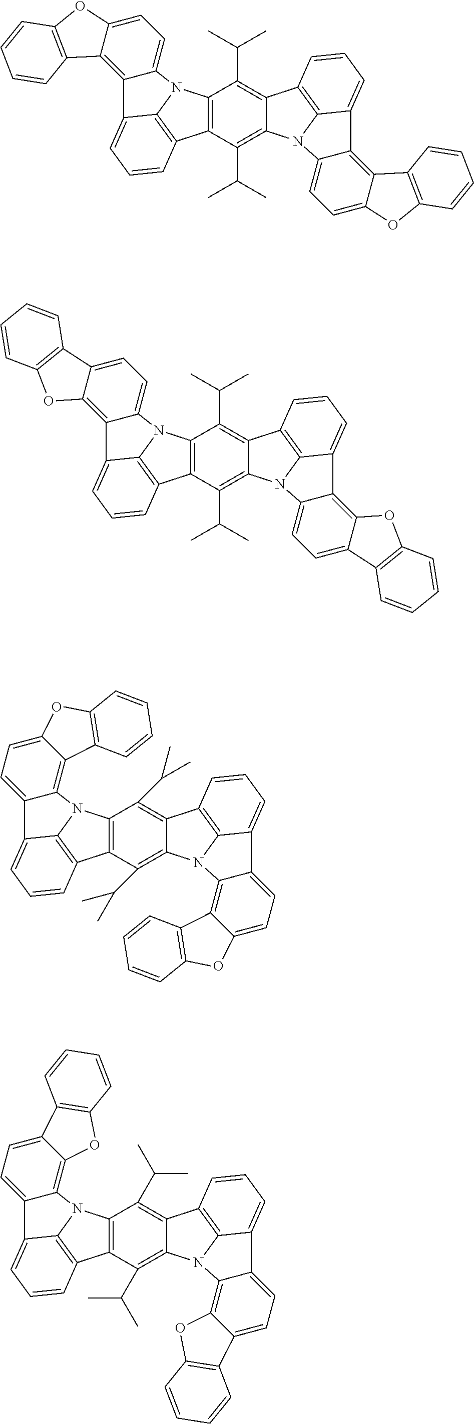 [SMILES: CC(C)c1c2c3cccc4c5c6c(ccc5n(c2c(C(C)C)c2c5cccc7c8c9c(ccc8n(c12)c57)oc1ccccc19)c34)oc1ccccc16 ; CC(C)c1c2c3cccc4c5c6oc7ccccc7c6ccc5n(c2c(C(C)C)c2c5cccc6c7c8oc9ccccc9c8ccc7n(c12)c56)c34 ; CC(C)c1c2c3cccc4c5ccc6oc7ccccc7c6c5n(c2c(C(C)C)c2c5cccc6c7ccc8oc9ccccc9c8c7n(c12)c65)c43 ; CC(C)c1c2c3cccc4c5ccc6c7ccccc7oc6c5n(c2c(C(C)C)c2c5cccc6c7ccc8c9ccccc9oc8c7n(c12)c65)c43]

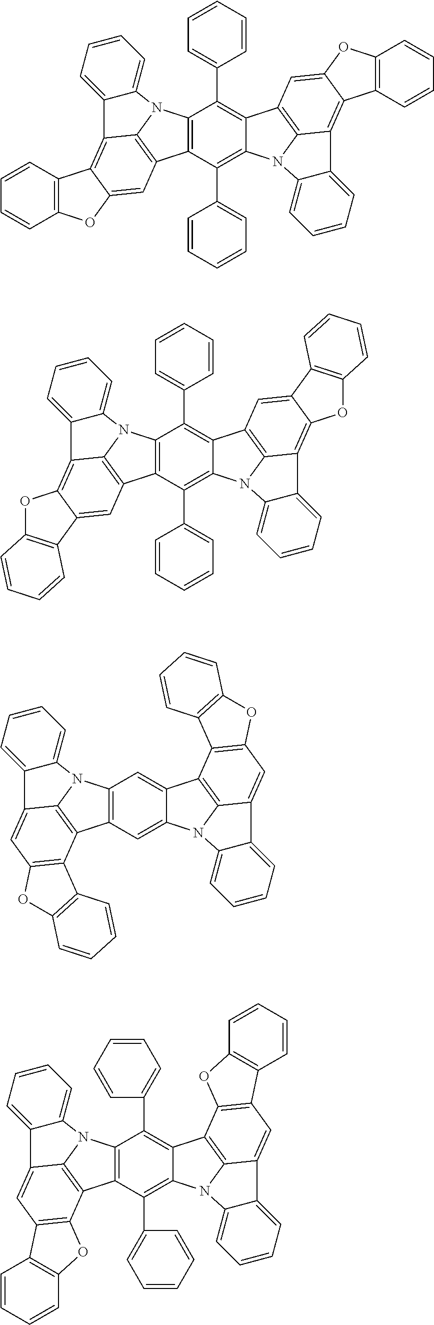 [SMILES: c1ccc(-c2c3c4cc5oc6ccccc6c5c5c6ccccc6n(c3c(-c3ccccc3)c3c6cc7oc8ccccc8c7c7c8ccccc8n(c23)c67)c45)cc1 ; c1ccc(-c2c3c4cc5c6ccccc6oc5c5c6ccccc6n(c3c(-c3ccccc3)c3c6cc7c8ccccc8oc7c7c8ccccc8n(c23)c67)c45)cc1 ; c1ccc2c(c1)oc1cc3c4ccccc4n4c5cc6c7c8c(cc9c%10ccccc%10n(c6cc5c(c12)c34)c97)oc1ccccc18 ; c1ccc(-c2c3c4c5oc6ccccc6c5cc5c6ccccc6n(c3c(-c3ccccc3)c3c6c7oc8ccccc8c7cc7c8ccccc8n(c23)c76)c54)cc1]

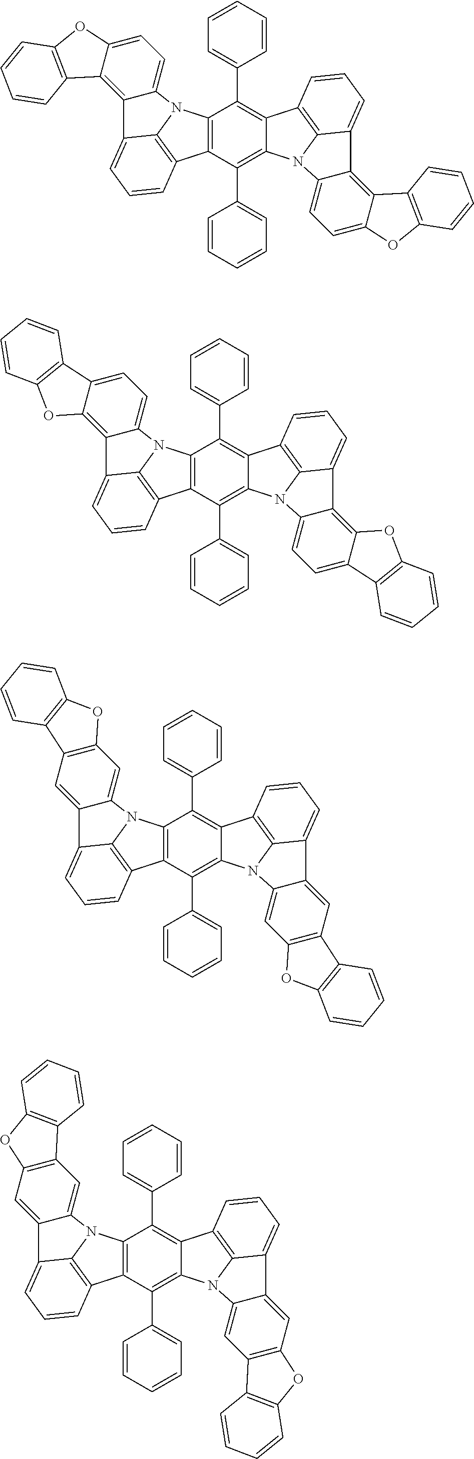 [SMILES: c1ccc(-c2c3c4cccc5c6c7c(ccc6n(c3c(-c3ccccc3)c3c6cccc8c9c%10c(ccc9n(c23)c68)oc2ccccc2%10)c45)oc2ccccc27)cc1 ; c1ccc(-c2c3c4cccc5c6c7oc8ccccc8c7ccc6n(c3c(-c3ccccc3)c3c6cccc7c8c9oc%10ccccc%10c9ccc8n(c23)c67)c45)cc1 ; c1ccc(-c2c3c4cccc5c6cc7c(cc6n(c3c(-c3ccccc3)c3c6cccc8c9cc%10c(cc9n(c23)c86)oc2ccccc2%10)c54)oc2ccccc27)cc1 ; c1ccc(-c2c3c4cccc5c6cc7oc8ccccc8c7cc6n(c3c(-c3ccccc3)c3c6cccc7c8cc9oc%10ccccc%10c9cc8n(c23)c76)c54)cc1]

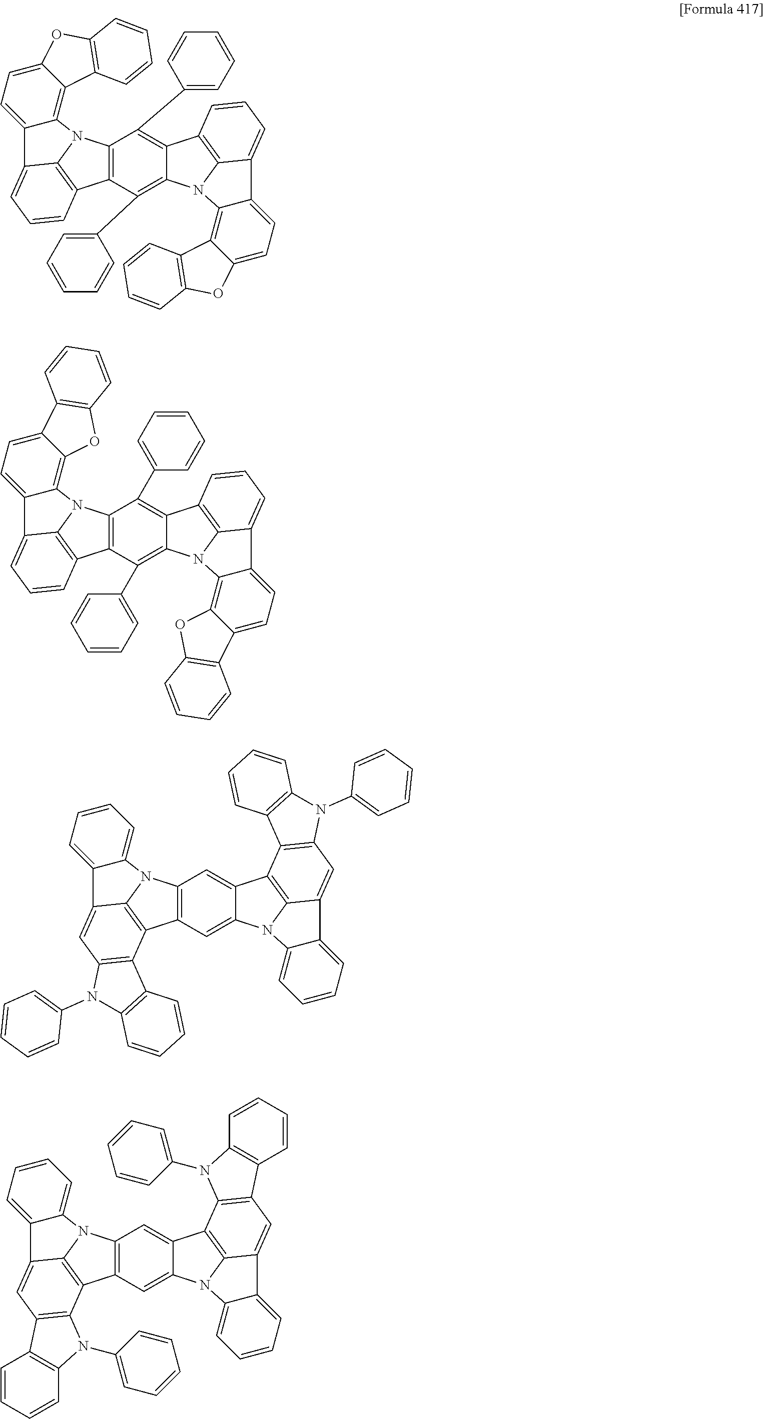 [SMILES: c1ccc(-c2c3c4cccc5c6ccc7oc8ccccc8c7c6n(c3c(-c3ccccc3)c3c6cccc7c8ccc9oc%10ccccc%10c9c8n(c23)c76)c54)cc1 ; c1ccc(-c2c3c4cccc5c6ccc7c8ccccc8oc7c6n(c3c(-c3ccccc3)c3c6cccc7c8ccc9c%10ccccc%10oc9c8n(c23)c76)c54)cc1 ; c1ccc(-n2c3ccccc3c3c4c5cc6c(cc5n5c7ccccc7c(cc32)c45)c2c3c4ccccc4n(-c4ccccc4)c3cc3c4ccccc4n6c32)cc1 ; c1ccc(-n2c3ccccc3c3cc4c5ccccc5n5c6cc7c8c9c(cc%10c%11ccccc%11n(c7cc6c(c32)c45)c%108)c2ccccc2n9-c2ccccc2)cc1]

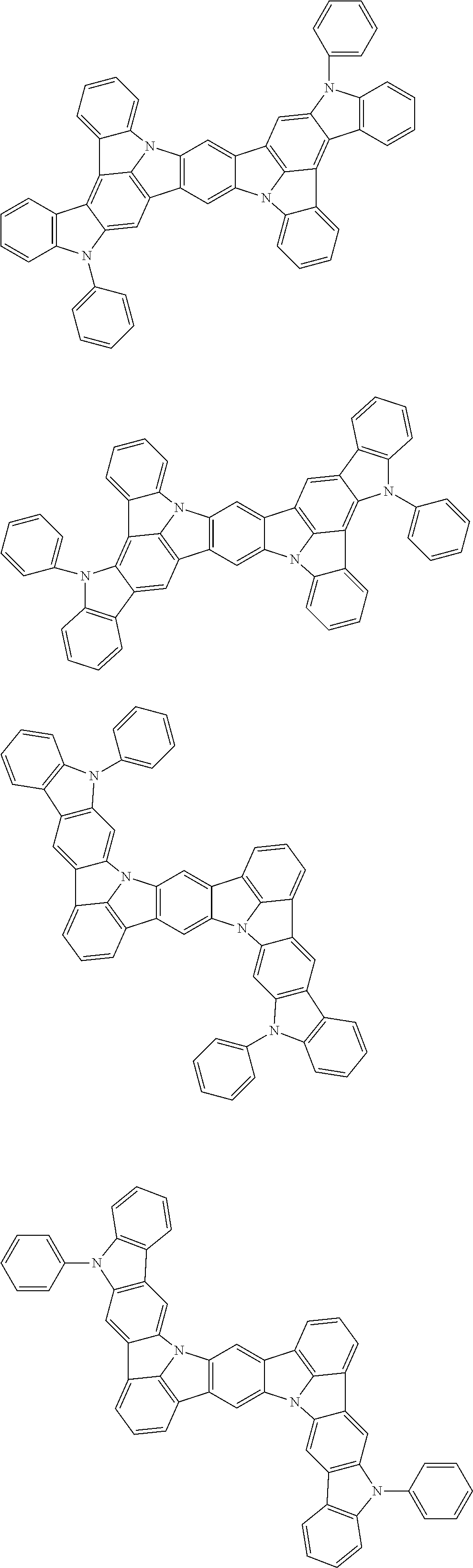 [SMILES: c1ccc(-n2c3ccccc3c3c4c5ccccc5n5c6cc7c8cc9c(c%10ccccc%10n9-c9ccccc9)c9c%10ccccc%10n(c7cc6c(cc32)c45)c89)cc1 ; c1ccc(-n2c3ccccc3c3cc4c5cc6c(cc5n5c7ccccc7c(c32)c45)c2cc3c4ccccc4n(-c4ccccc4)c3c3c4ccccc4n6c23)cc1 ; c1ccc(-n2c3ccccc3c3cc4c5cccc6c7cc8c(cc7n(c4cc32)c65)c2cccc3c4cc5c6ccccc6n(-c6ccccc6)c5cc4n8c23)cc1 ; c1ccc(-n2c3ccccc3c3cc4c(cc32)c2cccc3c5cc6c(cc5n4c23)c2cccc3c4cc5c(cc4n6c32)c2ccccc2n5-c2ccccc2)cc1]

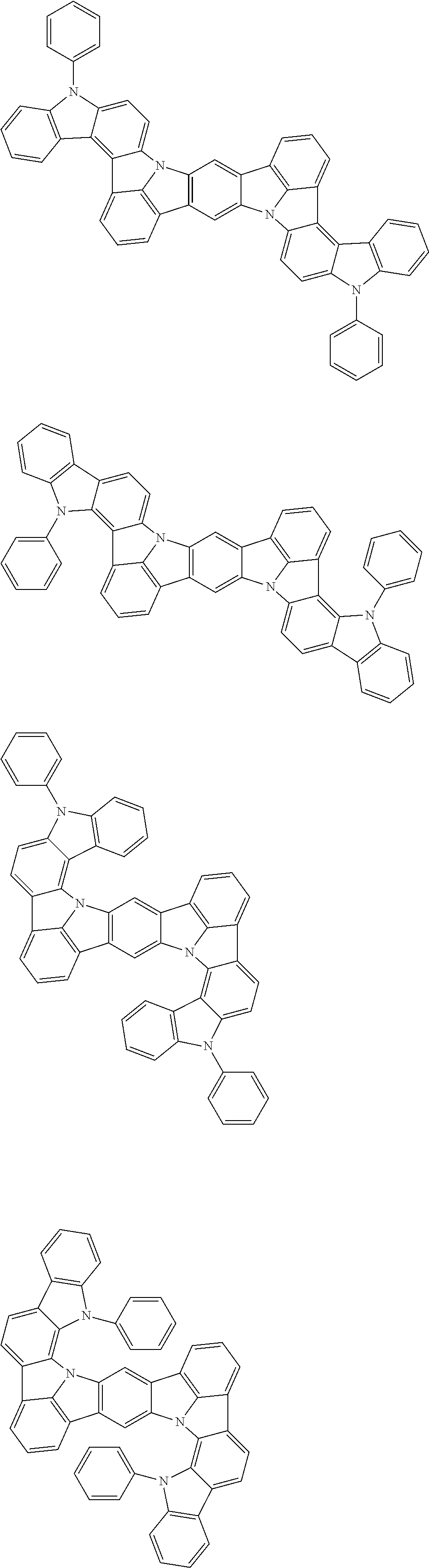 [SMILES: c1ccc(-n2c3ccccc3c3c4c5cccc6c7cc8c(cc7n(c4ccc32)c65)c2cccc3c4c5c6ccccc6n(-c6ccccc6)c5ccc4n8c23)cc1 ; c1ccc(-n2c3ccccc3c3ccc4c(c5cccc6c7cc8c(cc7n4c65)c4cccc5c6c7c(ccc6n8c45)c4ccccc4n7-c4ccccc4)c32)cc1 ; c1ccc(-n2c3ccccc3c3c2ccc2c4cccc5c6cc7c(cc6n(c54)c23)c2cccc3c4ccc5c(c6ccccc6n5-c5ccccc5)c4n7c23)cc1 ; c1ccc(-n2c3ccccc3c3ccc4c5cccc6c7cc8c(cc7n(c65)c4c32)c2cccc3c4ccc5c6ccccc6n(-c6ccccc6)c5c4n8c23)cc1]

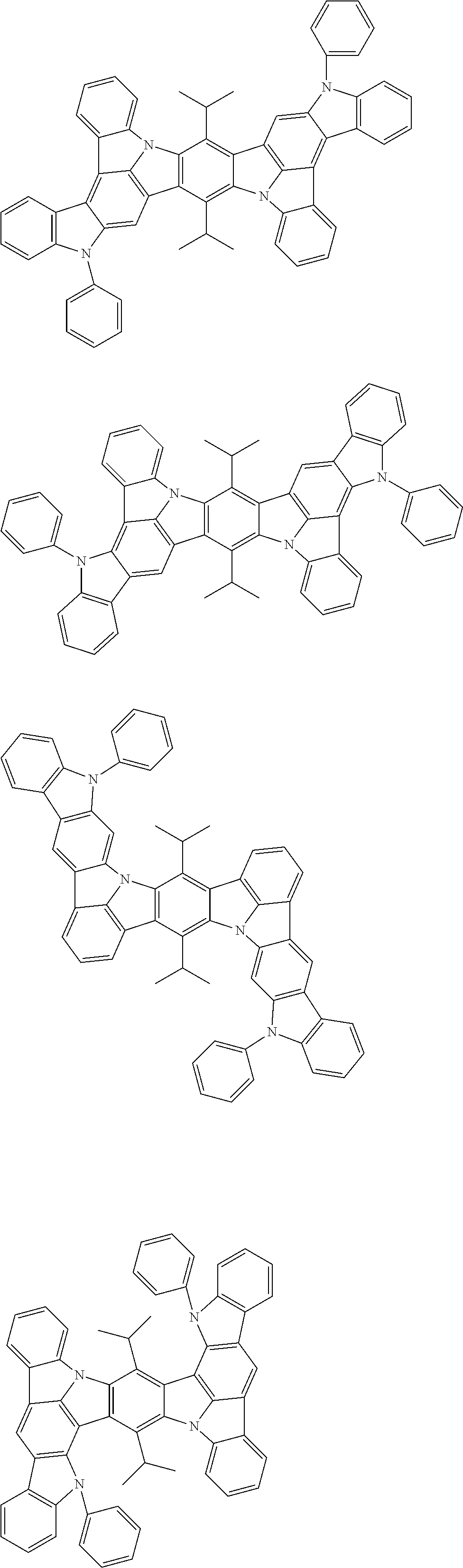 [SMILES: CC(C)c1c2c3cc4c(c5ccccc5n4-c4ccccc4)c4c5ccccc5n(c2c(C(C)C)c2c5cc6c(c7ccccc7n6-c6ccccc6)c6c7ccccc7n(c12)c56)c34 ; CC(C)c1c2c3cc4c5ccccc5n(-c5ccccc5)c4c4c5ccccc5n(c2c(C(C)C)c2c5cc6c7ccccc7n(-c7ccccc7)c6c6c7ccccc7n(c12)c56)c34 ; CC(C)c1c2c3cccc4c5cc6c7ccccc7n(-c7ccccc7)c6cc5n(c2c(C(C)C)c2c5cccc6c7cc8c9ccccc9n(-c9ccccc9)c8cc7n(c12)c65)c43 ; CC(C)c1c2c3c4c(cc5c6ccccc6n(c2c(C(C)C)c2c6c7c(cc8c9ccccc9n(c12)c86)c1ccccc1n7-c1ccccc1)c53)c1ccccc1n4-c1ccccc1]

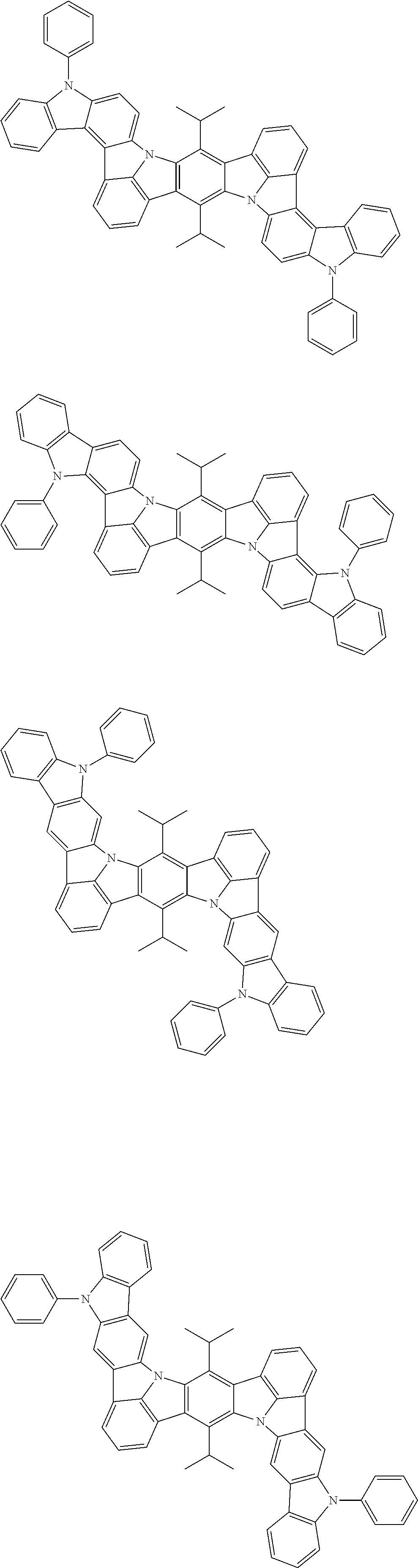 [SMILES: CC(C)c1c2c3cccc4c5c6c7ccccc7n(-c7ccccc7)c6ccc5n(c2c(C(C)C)c2c5cccc6c7c8c9ccccc9n(-c9ccccc9)c8ccc7n(c12)c56)c34 ; CC(C)c1c2c3cccc4c5c6c(ccc5n(c2c(C(C)C)c2c5cccc7c8c9c(ccc8n(c12)c57)c1ccccc1n9-c1ccccc1)c34)c1ccccc1n6-c1ccccc1 ; CC(C)c1c2c3cccc4c5cc6c7ccccc7n(-c7ccccc7)c6cc5n(c2c(C(C)C)c2c5cccc6c7cc8c9ccccc9n(-c9ccccc9)c8cc7n(c12)c65)c43 ; CC(C)c1c2c3cccc4c5cc6c(cc5n(c2c(C(C)C)c2c5cccc7c8cc9c(cc8n(c12)c75)c1ccccc1n9-c1ccccc1)c43)c1ccccc1n6-c1ccccc1]

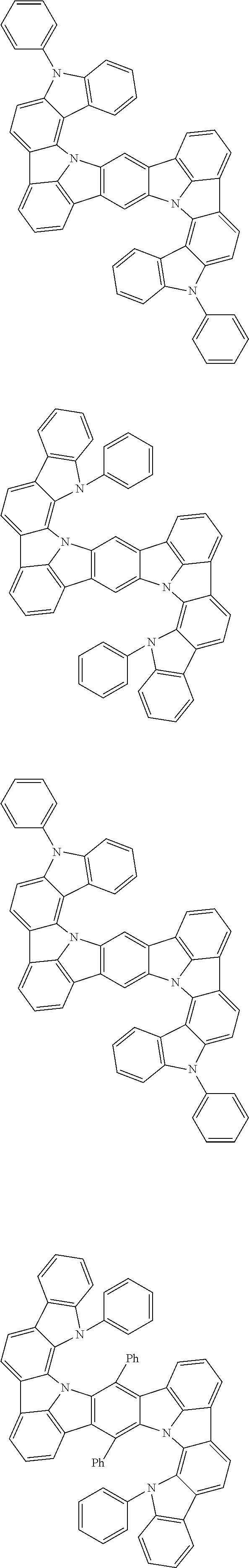 [SMILES: c1ccc(-n2c3ccccc3c3c2ccc2c4cccc5c6cc7c(cc6n(c54)c23)c2cccc3c4ccc5c(c6ccccc6n5-c5ccccc5)c4n7c23)cc1 ; c1ccc(-n2c3ccccc3c3ccc4c5cccc6c7cc8c(cc7n(c65)c4c32)c2cccc3c4ccc5c6ccccc6n(-c6ccccc6)c5c4n8c23)cc1 ; c1ccc(-n2c3ccccc3c3c2ccc2c4cccc5c6cc7c(cc6n(c54)c23)c2cccc3c4ccc5c(c6ccccc6n5-c5ccccc5)c4n7c23)cc1 ; c1ccc(-c2c3c4cccc5c6ccc7c8ccccc8n(-c8ccccc8)c7c6n(c3c(-c3ccccc3)c3c6cccc7c8ccc9c%10ccccc%10n(-c%10ccccc%10)c9c8n(c23)c76)c54)cc1]

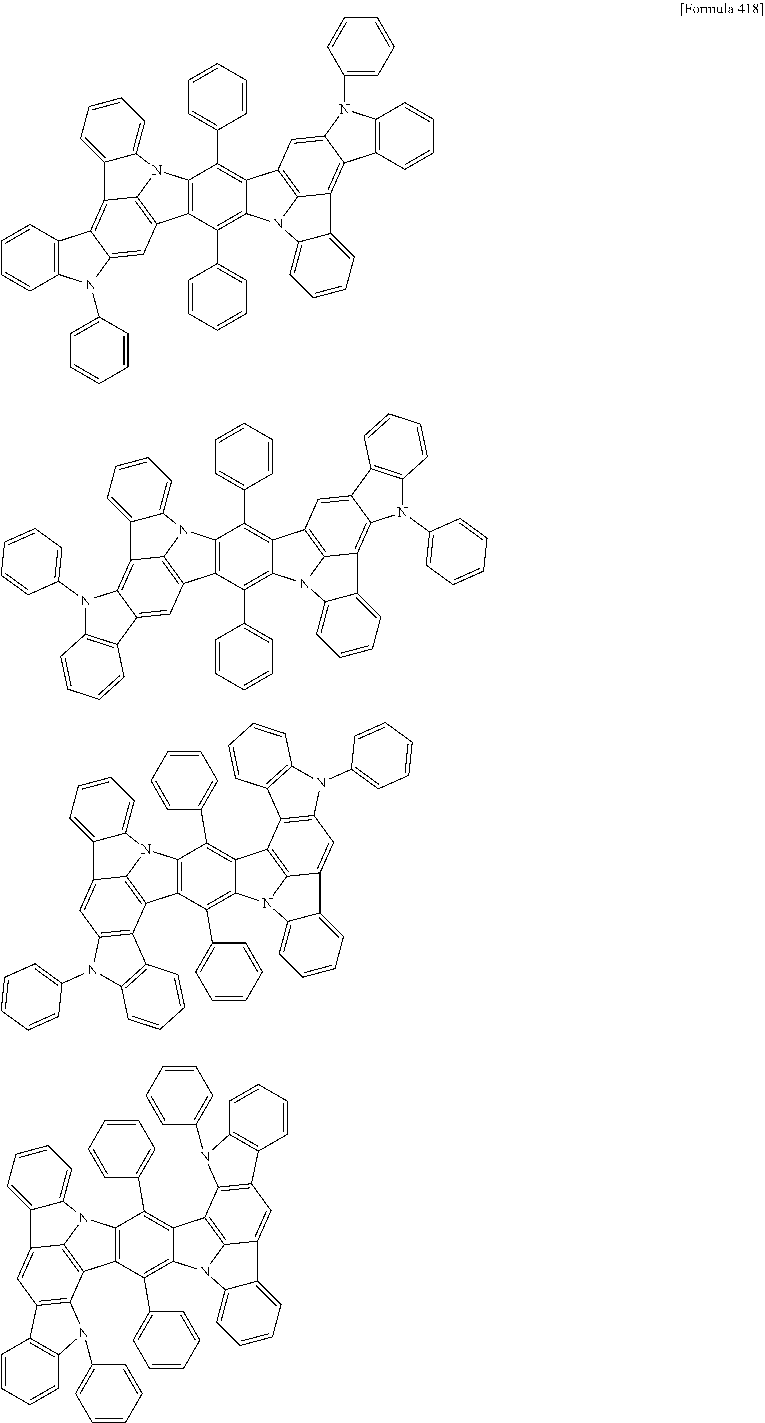

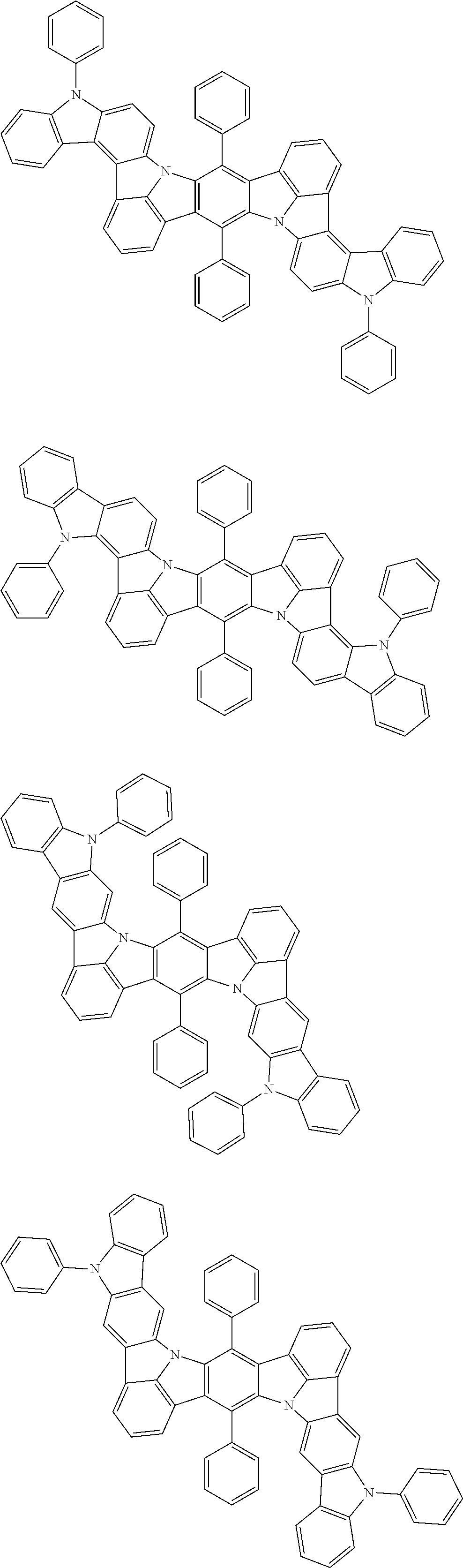

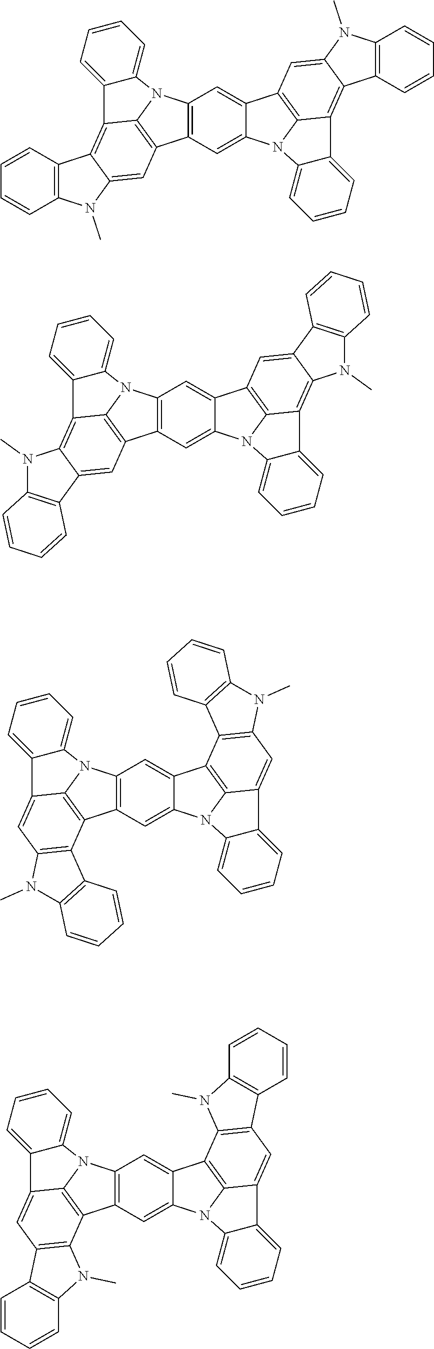

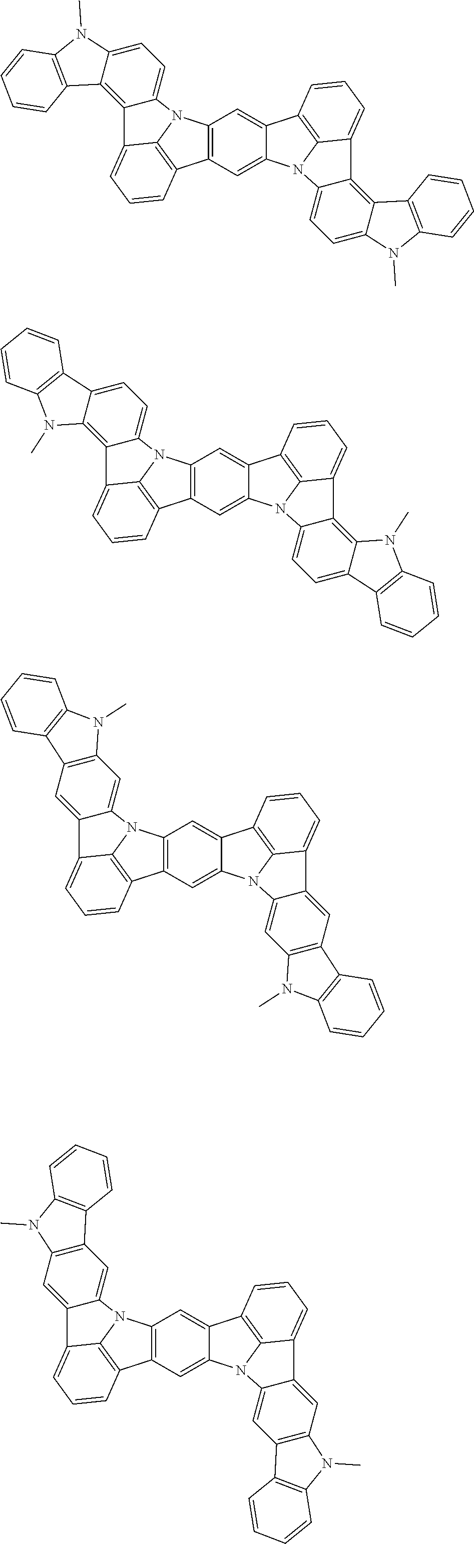

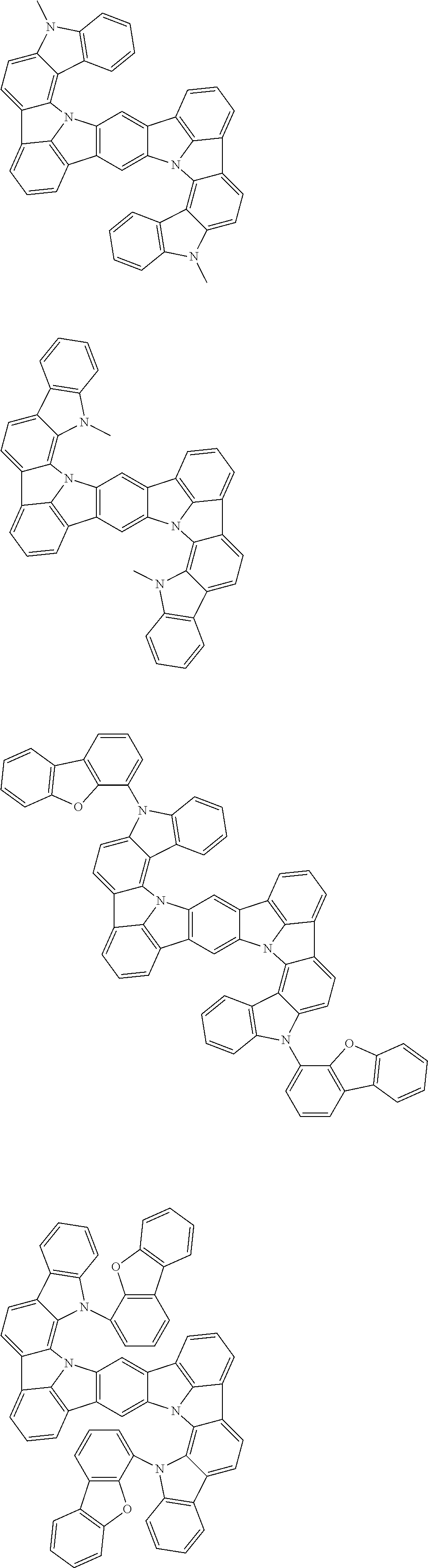 [SMILES: Cn1c2ccccc2c2c1ccc1c3cccc4c5cc6c(cc5n(c43)c12)c1cccc2c3ccc4c(c5ccccc5n4C)c3n6c12 ; Cn1c2ccccc2c2ccc3c4cccc5c6cc7c(cc6n(c54)c3c21)c1cccc2c3ccc4c5ccccc5n(C)c4c3n7c12 ; c1ccc2c(c1)oc1c(-n3c4ccccc4c4c3ccc3c5cccc6c7cc8c(cc7n(c65)c34)c3cccc4c5ccc6c(c7ccccc7n6-c6cccc7c6oc6ccccc67)c5n8c34)cccc12 ; c1ccc2c(c1)oc1c(-n3c4ccccc4c4ccc5c6cccc7c8cc9c(cc8n(c76)c5c43)c3cccc4c5ccc6c7ccccc7n(-c7cccc8c7oc7ccccc78)c6c5n9c34)cccc12]

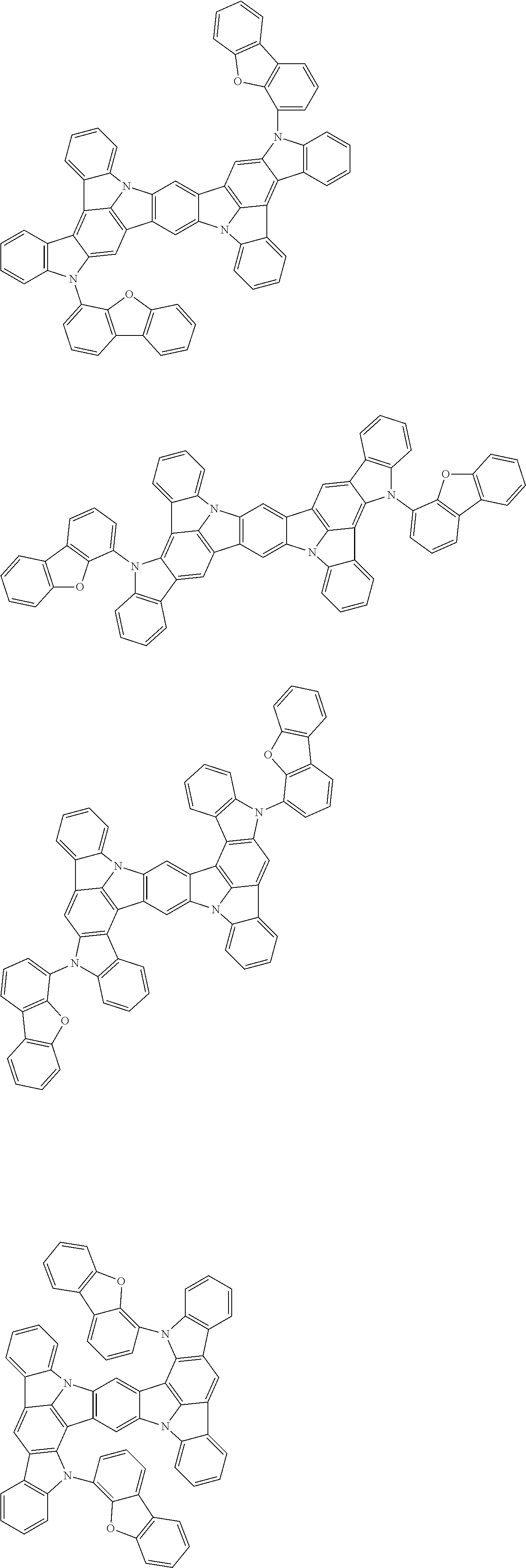

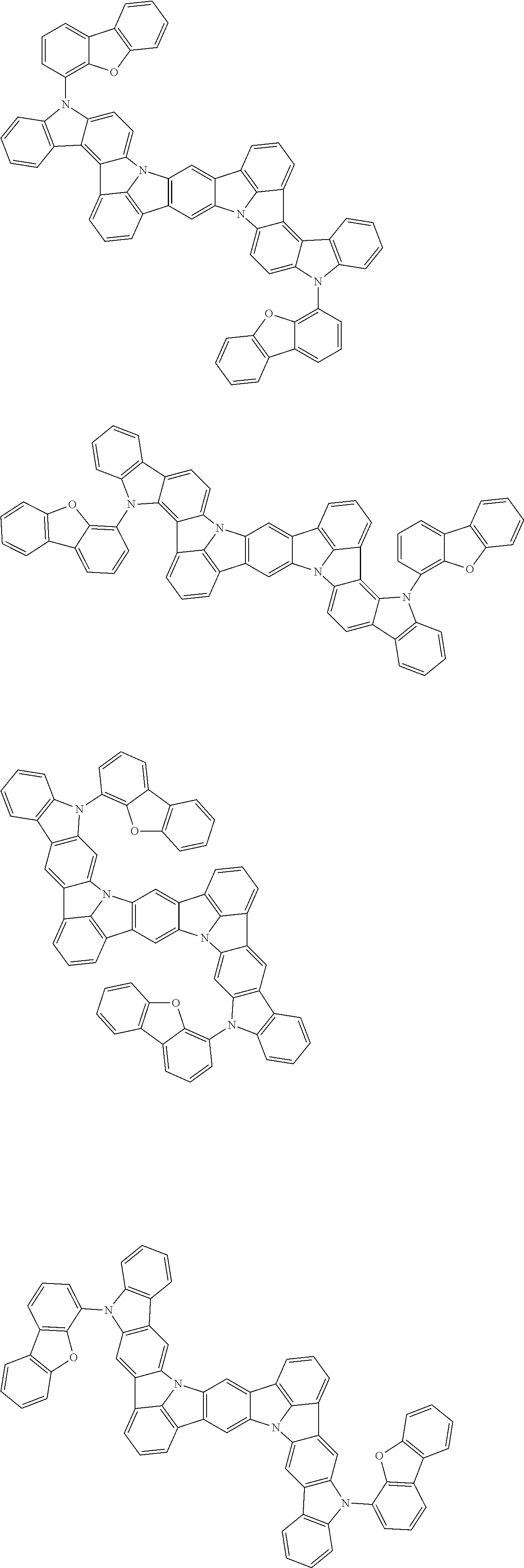 [SMILES: c1ccc2c(c1)oc1c(-n3c4ccccc4c4c5c6cccc7c8cc9c(cc8n(c5ccc43)c76)c3cccc4c5c6c7ccccc7n(-c7cccc8c7oc7ccccc78)c6ccc5n9c34)cccc12 ; c1ccc2c(c1)oc1c(-n3c4ccccc4c4ccc5c(c6cccc7c8cc9c(cc8n5c76)c5cccc6c7c8c(ccc7n9c56)c5ccccc5n8-c5cccc6c5oc5ccccc56)c43)cccc12 ; c1ccc2c(c1)oc1c(-n3c4ccccc4c4cc5c6cccc7c8cc9c(cc8n(c5cc43)c76)c3cccc4c5cc6c7ccccc7n(-c7cccc8c7oc7ccccc78)c6cc5n9c34)cccc12 ; c1ccc2c(c1)oc1c(-n3c4ccccc4c4cc5c(cc43)c3cccc4c6cc7c(cc6n5c34)c3cccc4c5cc6c(cc5n7c43)c3ccccc3n6-c3cccc4c3oc3ccccc34)cccc12]

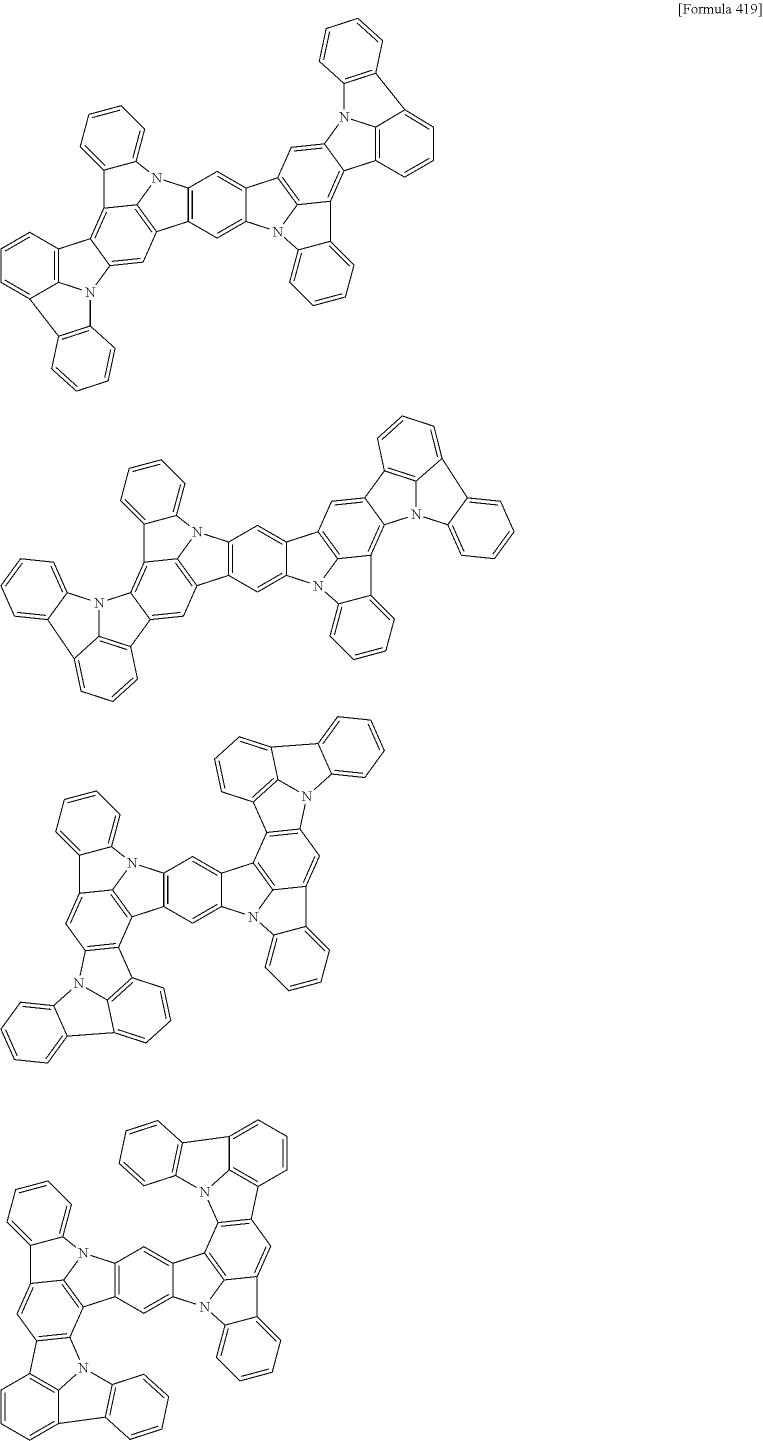 [SMILES: c1ccc2c(c1)c1cccc3c4c5c6ccccc6n6c7cc8c9cc%10c(c%11cccc%12c%13ccccc%13n%10c%12%11)c%10c%11ccccc%11n(c8cc7c(cc4n2c13)c56)c9%10 ; c1ccc2c(c1)c1c3c(cc4c5cccc6c7ccccc7n(c65)c41)c1cc4c(cc1n23)c1cc2c3cccc5c6ccccc6n(c53)c2c2c3ccccc3n4c12 ; c1ccc2c(c1)c1cccc3c4c5c6cc7c(cc6n6c8ccccc8c(cc4n2c13)c56)c1c2c3cccc4c5ccccc5n(c2cc2c5ccccc5n7c21)c43 ; c1ccc2c(c1)c1cc3c4cccc5c6ccccc6n(c54)c3c3c4cc5c(cc4n2c13)c1c2c(cc3c4cccc6c7ccccc7n(c64)c31)c1ccccc1n52]

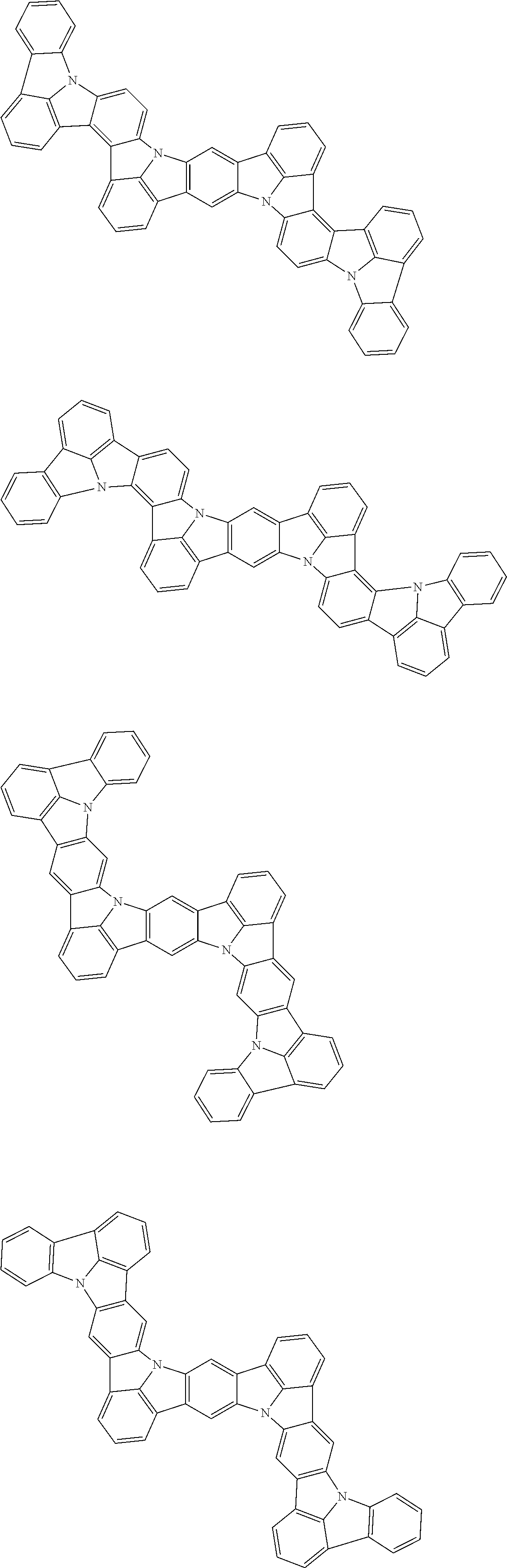 [SMILES: c1ccc2c(c1)c1cccc3c4c5c6cccc7c8cc9c(cc8n(c5ccc4n2c13)c76)c1cccc2c3c4c5cccc6c7ccccc7n(c4ccc3n9c12)c65 ; c1ccc2c(c1)c1cccc3c4ccc5c(c6cccc7c8cc9c(cc8n5c76)c5cccc6c7c(ccc8c%10cccc%11c%12ccccc%12n(c%11%10)c87)n9c56)c4n2c13 ; c1ccc2c(c1)c1cccc3c4cc5c6cccc7c8cc9c(cc8n(c5cc4n2c13)c76)c1cccc2c3cc4c5cccc6c7ccccc7n(c4cc3n9c12)c65 ; c1ccc2c(c1)c1cccc3c4cc5c(cc4n2c13)c1cccc2c3cc4c(cc3n5c12)c1cccc2c3cc5c(cc3n4c21)c1cccc2c3ccccc3n5c21]

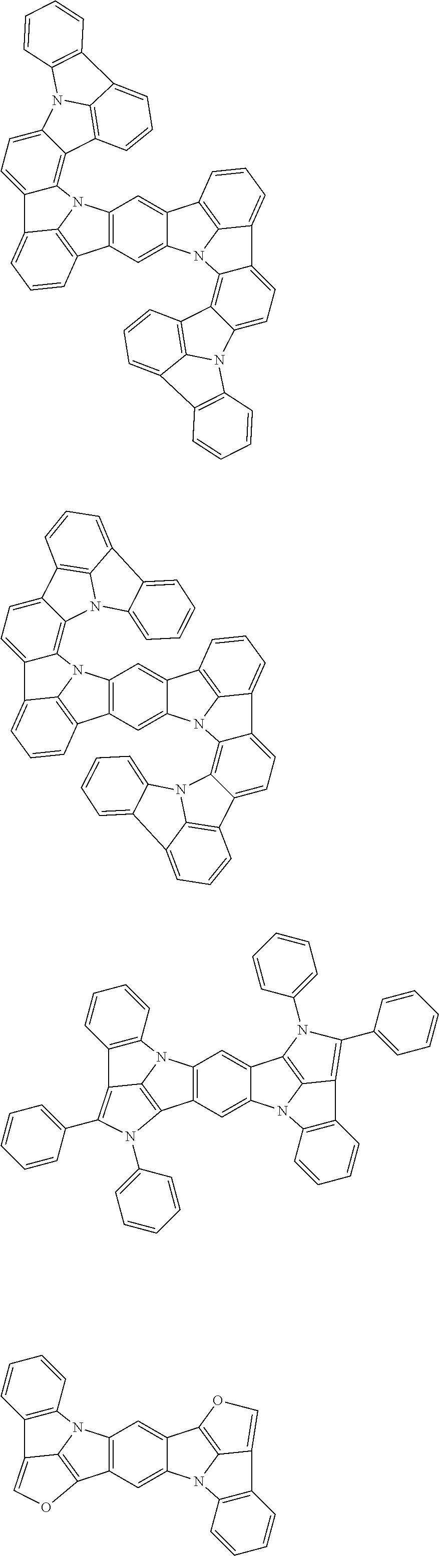 [SMILES: c1ccc2c(c1)c1cccc3c4c(ccc5c6cccc7c8cc9c(cc8n(c76)c54)c4cccc5c6ccc7c(c8cccc%10c%11ccccc%11n7c%108)c6n9c45)n2c13 ; c1ccc2c(c1)c1cccc3c4ccc5c6cccc7c8cc9c(cc8n(c76)c5c4n2c13)c1cccc2c3ccc4c5cccc6c7ccccc7n(c65)c4c3n9c12 ; c1ccc(-c2c3c4ccccc4n4c5cc6c7c8c(c(-c9ccccc9)n7-c7ccccc7)c7ccccc7n8c6cc5c(c34)n2-c2ccccc2)cc1 ; c1ccc2c(c1)c1coc3c4cc5c(cc4n2c13)c1occ2c3ccccc3n5c21]

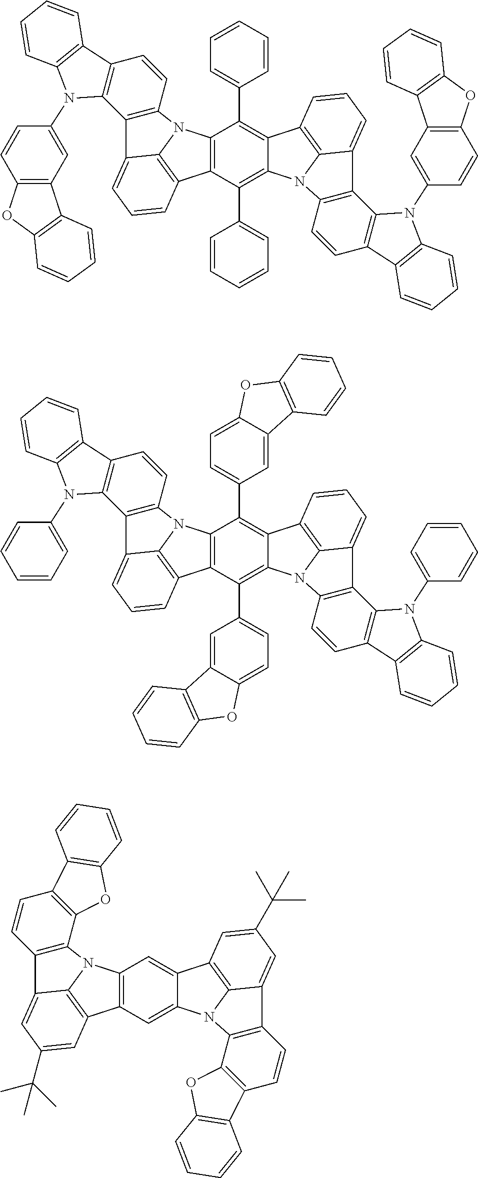 [SMILES: c1ccc(-c2c3c4cccc5c6c7c(ccc6n(c3c(-c3ccccc3)c3c6cccc8c9c%10c(ccc9n(c23)c68)c2ccccc2n%10-c2ccc3oc6ccccc6c3c2)c45)c2ccccc2n7-c2ccc3oc4ccccc4c3c2)cc1 ; c1ccc(-n2c3ccccc3c3ccc4c(c5cccc6c7c(-c8ccc9oc%10ccccc%10c9c8)c8c(c(-c9ccc%10oc%11ccccc%11c%10c9)c7n4c65)c4cccc5c6c7c(ccc6n8c45)c4ccccc4n7-c4ccccc4)c32)cc1 ; CC(C)(C)c1cc2c3cc4c(cc3n3c2c(c1)c1ccc2c5ccccc5oc2c13)c1cc(C(C)(C)C)cc2c3ccc5c6ccccc6oc5c3n4c12]

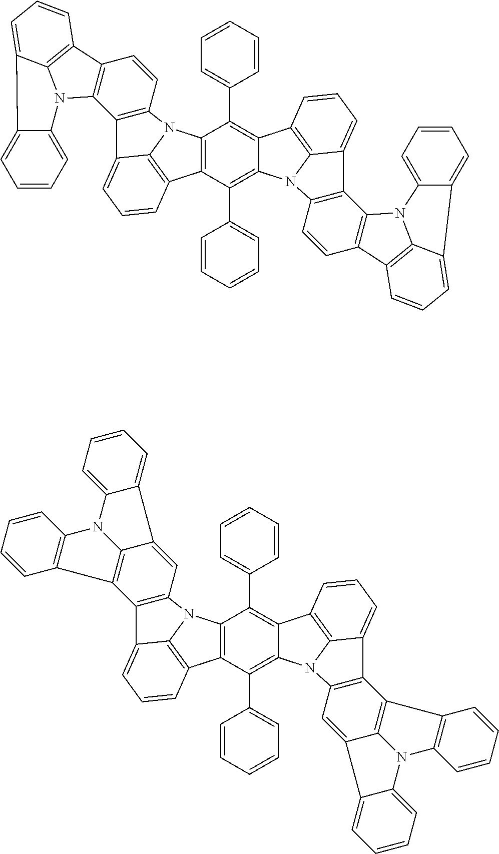 [SMILES: c1ccc(-c2c3c4cccc5c6c(ccc7c8cccc9c%10ccccc%10n(c98)c76)n(c3c(-c3ccccc3)c3c6cccc7c8c(ccc9c%10cccc%11c%12ccccc%12n(c%11%10)c98)n(c23)c67)c45)cc1 ; c1ccc(-c2c3c4cccc5c6c7c8ccccc8n8c9ccccc9c(cc6n(c3c(-c3ccccc3)c3c6cccc9c%10c%11c%12ccccc%12n%12c%13ccccc%13c(cc%10n(c23)c69)c%11%12)c45)c78)cc1]

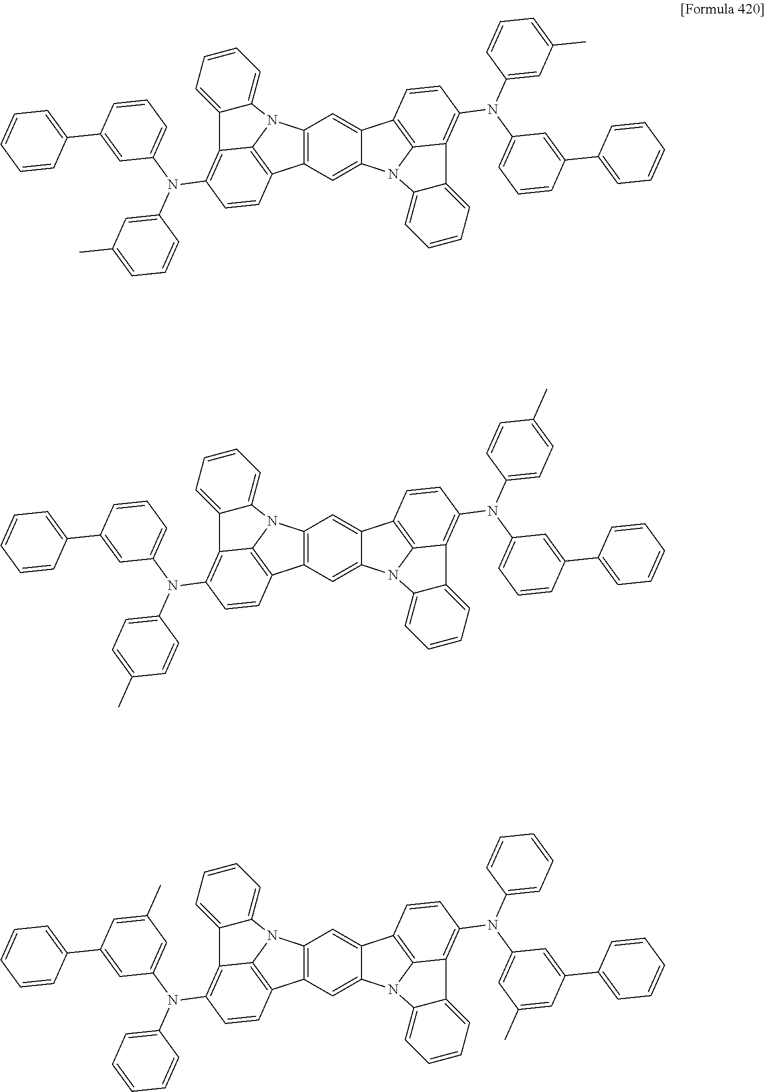 [SMILES: Cc1cccc(N(c2cccc(-c3ccccc3)c2)c2ccc3c4cc5c(cc4n4c6ccccc6c2c34)c2ccc(N(c3cccc(C)c3)c3cccc(-c4ccccc4)c3)c3c4ccccc4n5c23)c1 ; Cc1ccc(N(c2cccc(-c3ccccc3)c2)c2ccc3c4cc5c(cc4n4c6ccccc6c2c34)c2ccc(N(c3ccc(C)cc3)c3cccc(-c4ccccc4)c3)c3c4ccccc4n5c23)cc1 ; Cc1cc(-c2ccccc2)cc(N(c2ccccc2)c2ccc3c4cc5c(cc4n4c6ccccc6c2c34)c2ccc(N(c3ccccc3)c3cc(C)cc(-c4ccccc4)c3)c3c4ccccc4n5c23)c1]

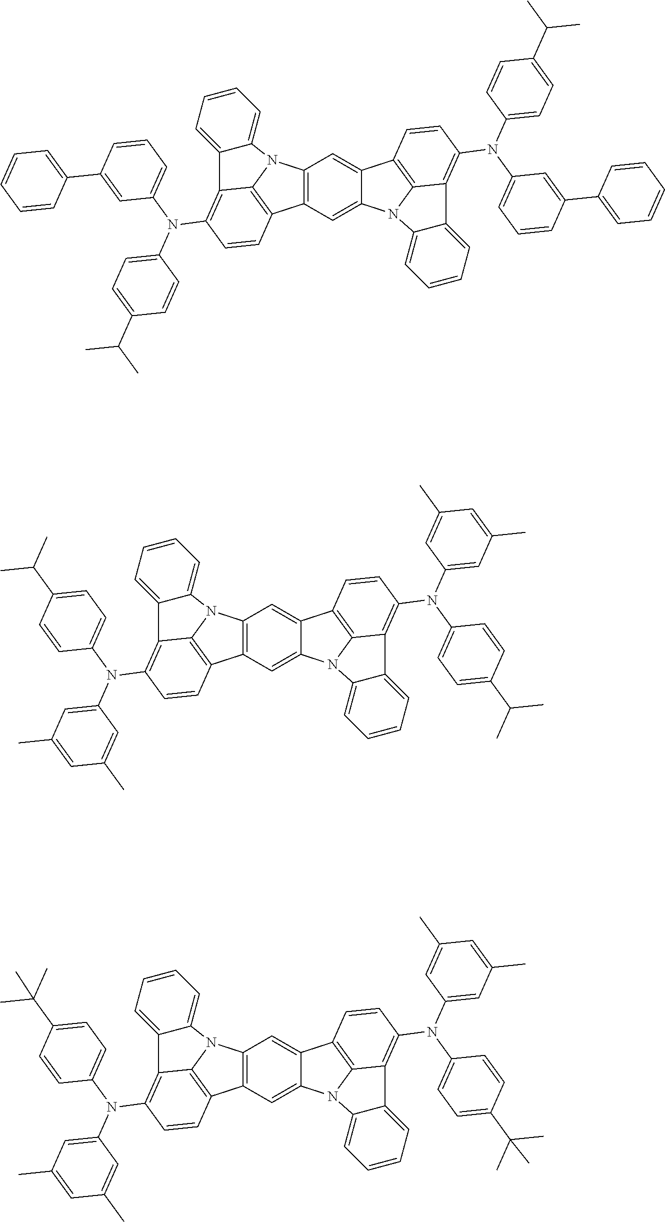

Compound Represented by Formula (6)

A compound represented by the formula (6) will be described.

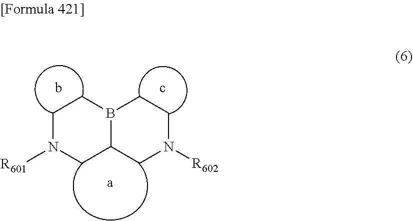

In the formula (6):
a ring, b ring and c ring are each independently a substituted or unsubstituted aromatic hydrocarbon ring having 6 to 50 ring carbon atoms, or a substituted or unsubstituted heterocycle having 5 to 50 ring atoms;
R₆₀₁ and R₆₀₂ are each independently bonded to the a ring, b ring or c ring to form a substituted or unsubstituted heterocycle, or not bonded thereto to form no substituted or unsubstituted heterocycle; and
R₆₀₁ and R₆₀₂ not forming the substituted or unsubstituted heterocycle are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

The a ring, b ring and c ring are each a ring (a substituted or unsubstituted aromatic hydrocarbon ring having 6 to 50 ring carbon atoms, or a substituted or unsubstituted heterocycle having 5 to 50 ring atoms) fused with a fused bicyclic structure formed of a boron atom and two nitrogen atoms at the center of the formula (6).

The “aromatic hydrocarbon ring” for the a, b, and c rings has the same structure as a compound formed by introducing a hydrogen atom to the “aryl group” described above.

Ring atoms of the “aromatic hydrocarbon ring” for the a ring include three carbon atoms on the fused bicyclic structure at the center of the formula (6).

Ring atoms of the “aromatic hydrocarbon ring” for the b ring and c ring include two carbon atoms on the fused bicyclic structure at the center of the formula (6).

Specific examples of the “substituted or unsubstituted aromatic hydrocarbon ring having 6 to 50 ring carbon atoms” include a compound formed by introducing a hydrogen atom to the “aryl group” described in the specific example group G1.

The “heterocycle” for the a, b, and c rings has the same structure as a compound formed by introducing a hydrogen atom to the “heterocyclic group” described above.

Ring atoms of the “heterocycle” for the a ring include three carbon atoms on the fused bicyclic structure at the center of the formula (6). Ring atoms of the “heterocycle” for the b ring and c ring include two carbon atoms on the fused bicyclic structure at the center of the formula (6). Specific examples of the “substituted or unsubstituted heterocycle having 5 to 50 ring atoms” include a compound formed by introducing a hydrogen atom to the “heterocyclic group” described in the specific example group G2.

R₆₀₁ and R₆₀₂ are optionally each independently bonded with the a ring, b ring, or c ring to form a substituted or unsubstituted heterocycle. The “heterocycle” in this arrangement includes a nitrogen atom on the fused bicyclic structure at the center of the formula (6). The heterocycle in the above arrangement optionally includes a hetero atom other than the nitrogen atom. R₆₀₁ and R₆₀₂ bonded with the a ring, b ring, or c ring specifically means that atoms forming R₆₀₁ and R₆₀₂ are bonded with atoms forming the a ring, b ring, or c ring. For instance, R₆₀₁ may be bonded with the a ring to form a bicyclic (or tri-or-more cyclic) fused nitrogen-containing heterocycle, in which the ring including R₆₀₁ and the a ring are fused. Specific examples of the nitrogen-containing heterocycle include a compound corresponding to a nitrogen-containing bi(or-more)cyclic fused heterocyclic group in the specific example group G2.

The same applies to R₆₀₁ bonded with the b ring, R₆₀₂ bonded with the a ring, and R₆₀₂ bonded with the c ring.

In an exemplary embodiment, the a ring, b ring and c ring in the formula (6) are each independently a substituted or unsubstituted aromatic hydrocarbon ring having 6 to 50 ring carbon atoms.

In an exemplary embodiment, the a ring, b ring and c ring in the formula (6) are each independently a substituted or unsubstituted benzene ring or a substituted or unsubstituted naphthalene ring.

In an exemplary embodiment, R₆₀₁ and R₆₀₂ in the formula (6) are each independently a substituted or unsubstituted aryl group having 6 to 50 ring carbon atoms, or a substituted or unsubstituted heterocyclic group having 5 to 50 ring atoms, preferably a substituted or unsubstituted aryl group having 6 to 50 ring carbon atoms.

In an exemplary embodiment, a compound represented by the formula (6) is a compound represented by a formula (62) below.

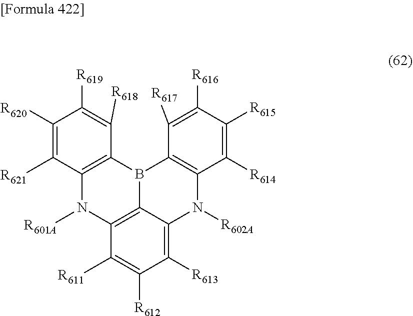

In the formula (62):
R_{601A} is bonded with at least one of R₆₁₁ or R₆₂₁ to form a substituted or unsubstituted heterocycle, or not bonded therewith to form no substituted or unsubstituted heterocycle;
R_{602A} is bonded with at least one of R₆₁₃ or R₆₁₄ to form a substituted or unsubstituted heterocycle, or not bonded therewith to form no substituted or unsubstituted heterocycle;
R_{601A} and R_{602A} not forming the substituted or unsubstituted heterocycle are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
at least one combination of adjacent two or more of R₆₁₁ to R₆₂₁ may be mutually bonded to form a substituted or unsubstituted monocyclic ring, mutually bonded to form a substituted or unsubstituted fused ring, or not mutually bonded; and
R₆₁₁ to R₆₂₁ not forming the substituted or unsubstituted heterocycle, the substituted or unsubstituted monocyclic ring, and the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group Represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.
R_{601A} and R_{602A} in the formula (62) are groups corresponding to R₆₀₁ and R₆₀₂ in the formula (6), respectively.

For instance, R_{601A} and R₆₁₁ are optionally bonded with each other to form a bicyclic (or tri-or-more cyclic) fused nitrogen-containing heterocycle, in which the ring including R_{601A} and R₆₁₁ and a benzene ring corresponding to the a ring are fused. Specific examples of the nitrogen-containing heterocycle include a compound corresponding to a nitrogen-containing bi(or-more)cyclic fused heterocyclic group in the specific example group G2. The same applies to R_{601A} bonded with R₆₂₁, R_{602A} bonded with R₆₁₃, and R_{602A} bonded with R₆₁₄.

At least one combination of adjacent two or more of R₆₁₁ to R₆₂₁ may be mutually bonded to form a substituted or unsubstituted monocyclic ring, or mutually bonded to form a substituted or unsubstituted fused ring.

For instance, R₆₁₁ and R₆₁₂ are optionally mutually bonded to form a structure in which a benzene ring, indole ring, pyrrole ring, benzofuran ring, benzothiophene ring or the like is fused to the six-membered ring bonded with R₆₁₁ and R₆₁₂, the resultant fused ring forming a naphthalene ring, carbazole ring, indole ring, dibenzofuran ring, or dibenzothiophene ring, respectively.

In an exemplary embodiment, R₆₁₁ to R₆₂₁ not contributing to ring formation are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In an exemplary embodiment, R₆₁₁ to R₆₂₁ not contributing to ring formation are each independently a hydrogen atom, a substituted or unsubstituted aryl group having 6 to 50 ring carbon atoms, or a substituted or unsubstituted heterocyclic group having 5 to 50 ring atoms.

In an exemplary embodiment, R₆₁₁ to R₆₂₁ not contributing to ring formation are each independently a hydrogen atom, or a substituted or unsubstituted alkyl group having 1 to 50 carbon atoms.

In an exemplary embodiment, R₆₁₁ to R₆₂₁ not contributing to ring formation are each independently a hydrogen atom, or a substituted or unsubstituted alkyl group having 1 to 50 carbon atoms, and
at least one of R₆₁₁ to R₆₂₁ is a substituted or unsubstituted alkyl group having 1 to 50 carbon atoms.

In an exemplary embodiment, a compound represented by the formula (62) is a compound represented by a formula (63) below.

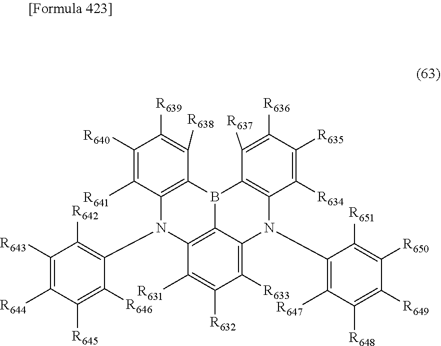

In the formula (63):
R₆₃₁ is bonded with R₆₄₆ to form a substituted or unsubstituted heterocycle, or not bonded therewith to form no substituted or unsubstituted heterocycle;
R₆₃₃ is bonded with R₆₄₇ to form a substituted or unsubstituted heterocycle, or not bonded therewith to form no substituted or unsubstituted heterocycle;
R₆₃₄ is bonded with R₆₅₁ to form a substituted or unsubstituted heterocycle, or not bonded therewith to form no substituted or unsubstituted heterocycle;
R₆₄₁ is bonded with R₆₄₂ to form a substituted or unsubstituted heterocycle, or not bonded therewith to form no substituted or unsubstituted heterocycle;
at least one combination of adjacent two or more of R₆₃₁ to R₆₅₁ are mutually bonded to form a substituted or unsubstituted monocyclic ring, mutually bonded to form a substituted or unsubstituted fused ring, or not mutually bonded;
R₆₃₁ to R₆₅₁ not forming the substituted or unsubstituted heterocycle, the substituted or unsubstituted monocyclic ring, and the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group Represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

R₆₃₁ are optionally bonded with R₆₄₆ to form a substituted or unsubstituted heterocycle. For instance, R₆₃₁ and R₆₄₆ are optionally bonded with each other to form a tri-or-more cyclic fused nitrogen-containing heterocycle, in which a benzene ring bonded with R₆₄₆, a ring including a nitrogen atom, and a benzene ring corresponding to the a ring are fused. Specific examples of the nitrogen-containing heterocycle include a compound corresponding to a nitrogen-containing tri(-or-more)cyclic fused heterocyclic group in the specific example group G2. The same applies to R₆₃₃ bonded with R₆₄₇, R₆₃₄ bonded with R₆₅₁, and R₆₄₁ bonded with R₆₄₂.

In an exemplary embodiment, R₆₃₁ to R₆₅₁ not contributing to ring formation are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In an exemplary embodiment, R₆₃₁ to R₆₅₁ not contributing to ring formation are each independently a hydrogen atom, a substituted or unsubstituted aryl group having 6 to 50 ring carbon atoms, or a substituted or unsubstituted heterocyclic group having 5 to 50 ring atoms.

In an exemplary embodiment, R₆₃₁ to R₆₅₁ not contributing to ring formation are each independently a hydrogen atom, or a substituted or unsubstituted alkyl group having 1 to 50 carbon atoms.

In an exemplary embodiment, R₆₃₁ to R₆₅₁ not contributing to ring formation are each independently a hydrogen atom, or a substituted or unsubstituted alkyl group having 1 to 50 carbon atoms, and
at least one of R₆₃₁ to R₆₅₁ is a substituted or unsubstituted alkyl group having 1 to 50 carbon atoms.

In an exemplary embodiment, a compound represented by the formula (63) is a compound represented by a formula (63A) below.

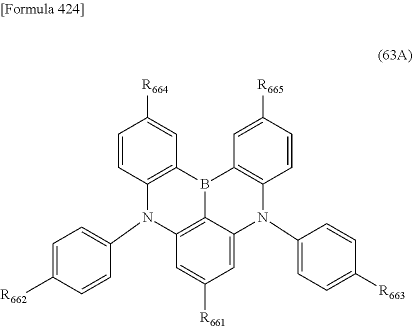

In the formula (63A):
R₆₆₁ is a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms, and
R₆₆₂ to R₆₆₅ are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms.

In an exemplary embodiment, R₆₆₁ to R₆₆₅ are each independently a substituted or unsubstituted alkyl group having 1 to 50 carbon atoms, or a substituted or unsubstituted aryl group having 6 to 50 ring carbon atoms.

In an exemplary embodiment, R₆₆₁ to R₆₆₅ are each independently a substituted or unsubstituted alkyl group having 1 to 50 carbon atoms.

In an exemplary embodiment, a compound represented by the formula (63) is a compound represented by a formula (63B) below.

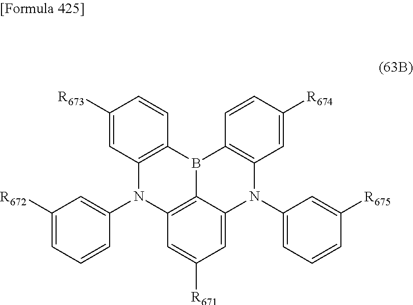

In the formula (63B):
R₆₇₁ and R₆₇₂ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —N(R₉₀₆)(R₉₀₇), or a substituted or unsubstituted aryl group having 6 to 50 ring carbon atoms; and
R₆₇₃ to R₆₇₅ are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —N(R₉₀₆)(R₉₀₇), or a substituted or unsubstituted aryl group having 6 to 50 ring carbon atoms.

In an exemplary embodiment, a compound represented by the formula (63) is a compound represented by a formula (63B′) below.

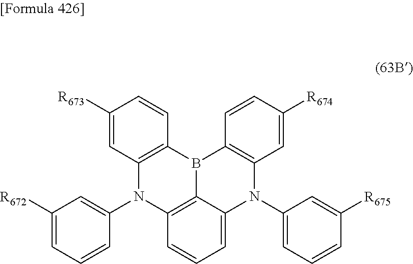

In the formula (63B′), R₆₇₂ to R₆₇₅ each independently represent the same as R₆₇₂ to R₆₇₅ in the formula (63B).

In an exemplary embodiment, at least one of R₆₇₁ to R₆₇₅ is a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —N(R₉₀₆)(R₉₀₇), or a substituted or unsubstituted aryl group having 6 to 50 ring carbon atoms.

In an exemplary embodiment: R₆₇₂ is a hydrogen atom, a substituted or unsubstituted alkyl group having 1 to 50 carbon atoms, a group represented by —N(R₉₀₆)(R₉₀₇), or a substituted or unsubstituted aryl group having 6 to 50 ring carbon atoms; and
R₆₇₁ and R₆₇₃ to R₆₇₅ are each independently a substituted or unsubstituted alkyl group having 1 to 50 carbon atoms, a group represented by —N(R₉₀₆)(R₉₀₇), or a substituted or unsubstituted aryl group having 6 to 50 ring carbon atoms.

In an exemplary embodiment, a compound represented by the formula (63) is a compound represented by a formula (63C) below.

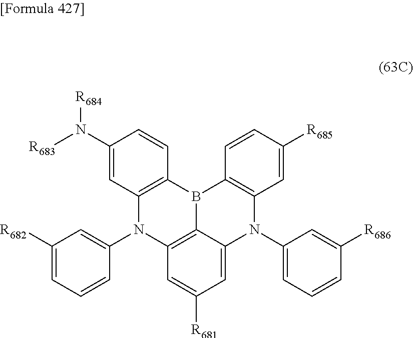

In the formula (63C): R₆₈₁ and R₆₈₂ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms; and
R₆₈₃ to R₆₈₆ are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms.

In an exemplary embodiment, a compound represented by the formula (63) is a compound represented by a formula (63C′) below.

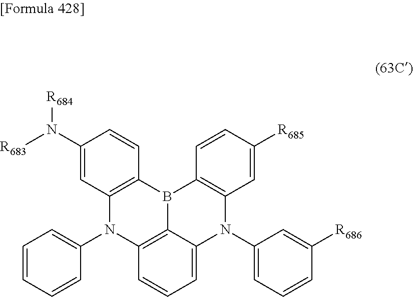

In the formula (63C′), R₆₈₃ to R₆₈₆ each independently represent the same as R₆₈₃ to R₆₈₆ in the formula (63C).

In an exemplary embodiment, R₆₈₁ to R₆₈₆ are each independently a substituted or unsubstituted alkyl group having 1 to 50 carbon atoms, or a substituted or unsubstituted aryl group having 6 to 50 ring carbon atoms.

In an exemplary embodiment, R₆₈₁ to R₆₈₆ are each independently a substituted or unsubstituted aryl group having 6 to 50 ring carbon atoms.

A compound represented by the formula (6) is producible by initially bonding the a ring, b ring and c ring with linking groups (a group including N—R₆₀₁ and a group including N—R₆₀₂) to form an intermediate (first reaction), and bonding the a ring, b ring and c ring with a linking group (a group including a boron atom) to form a final product (second reaction). In the first reaction, an amination reaction (e.g. Buchwald-Hartwig reaction) is applicable. In the second reaction, Tandem Hetero-Friedel-Crafts Reactions or the like is applicable.

Specific examples of a compound represented by the formula (6) are shown below. It should however be noted that these specific examples are merely exemplary and do not limit a compound represented by the formula (6).

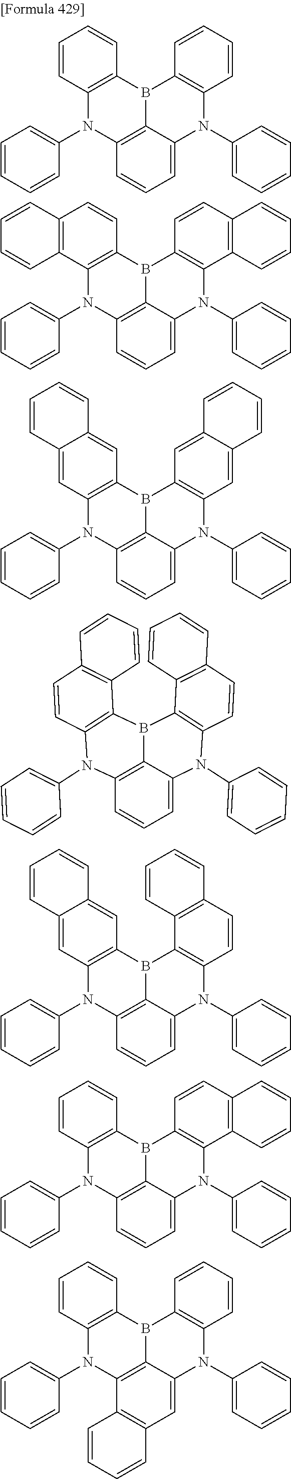

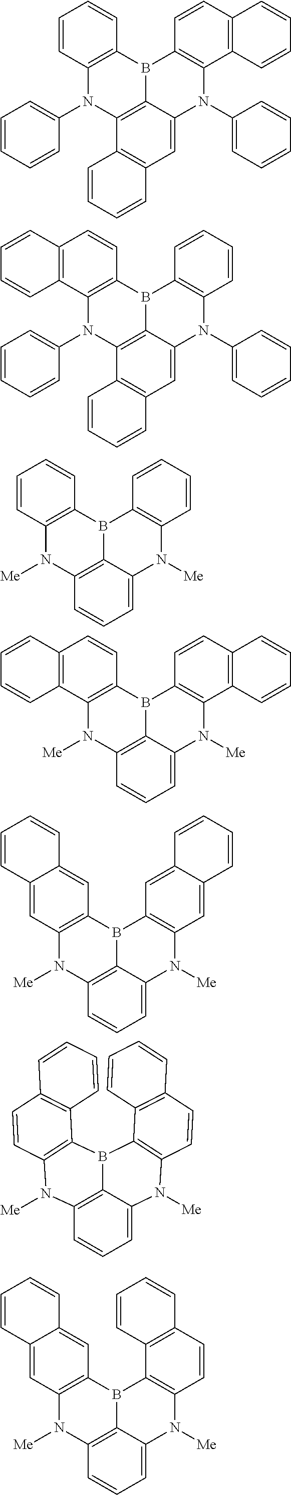

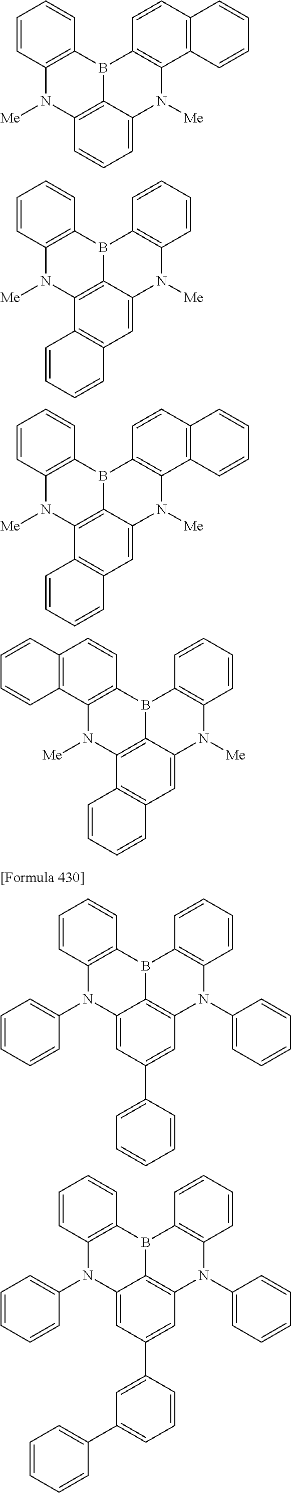

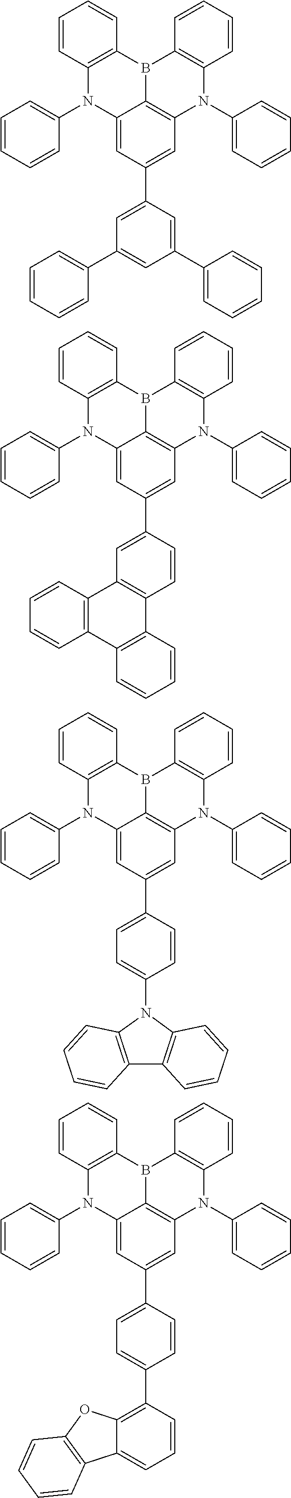

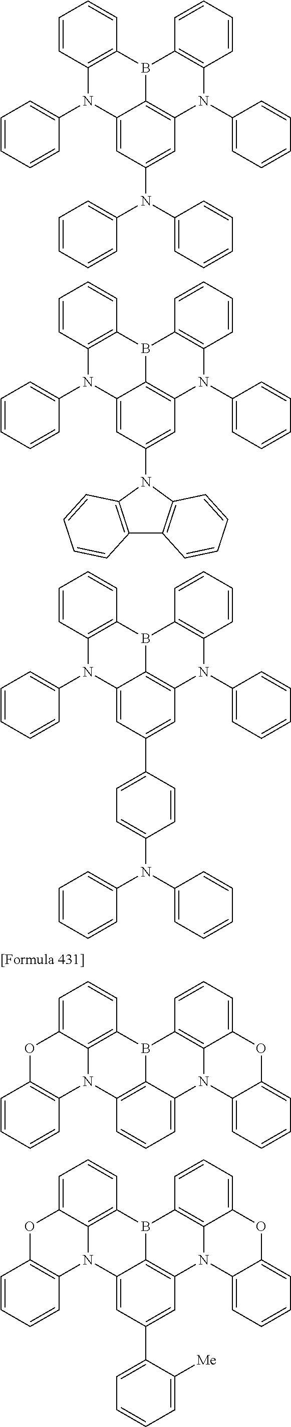

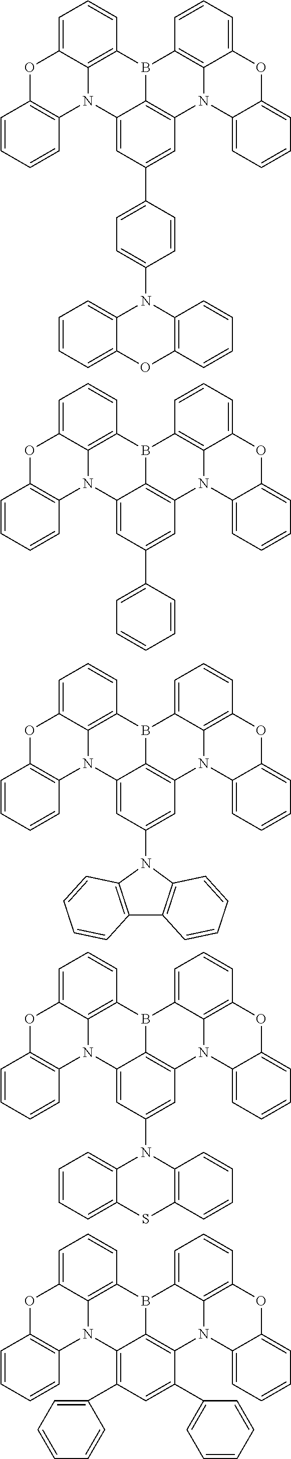

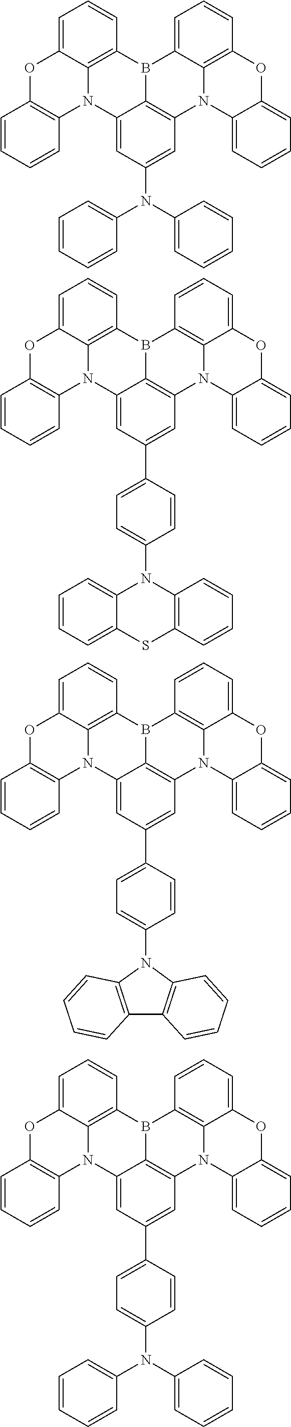

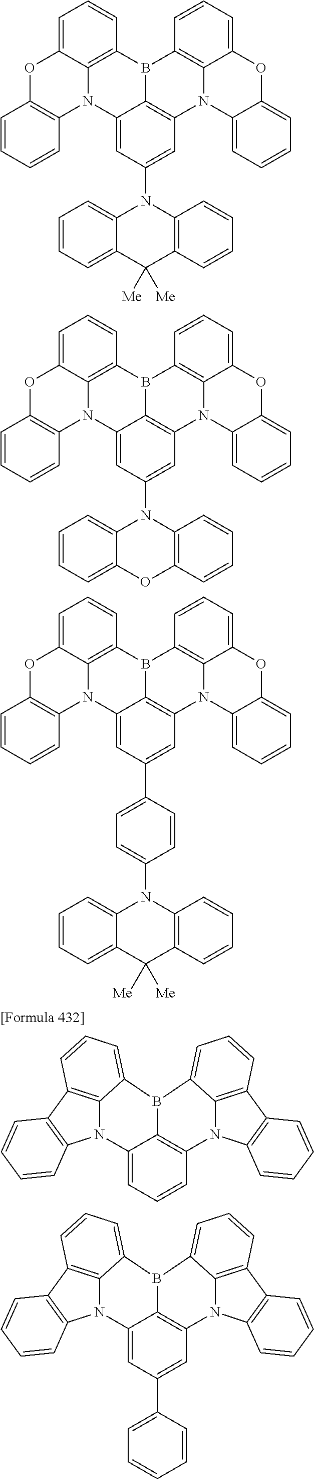

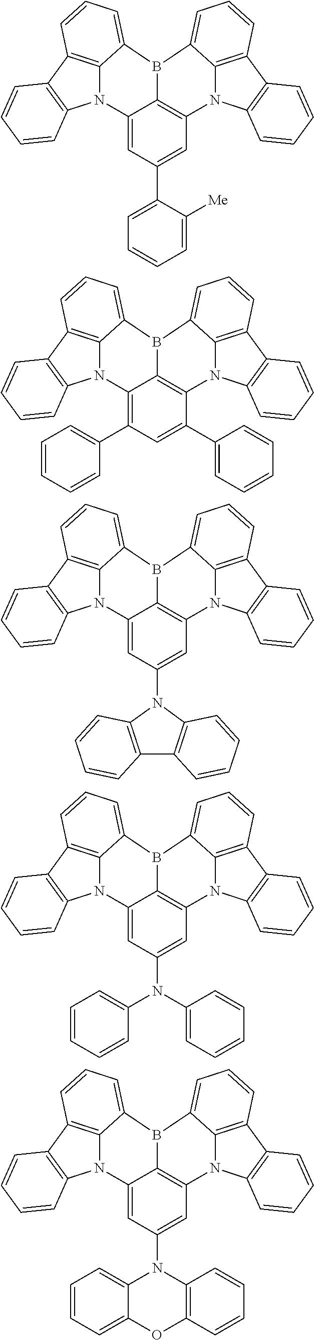

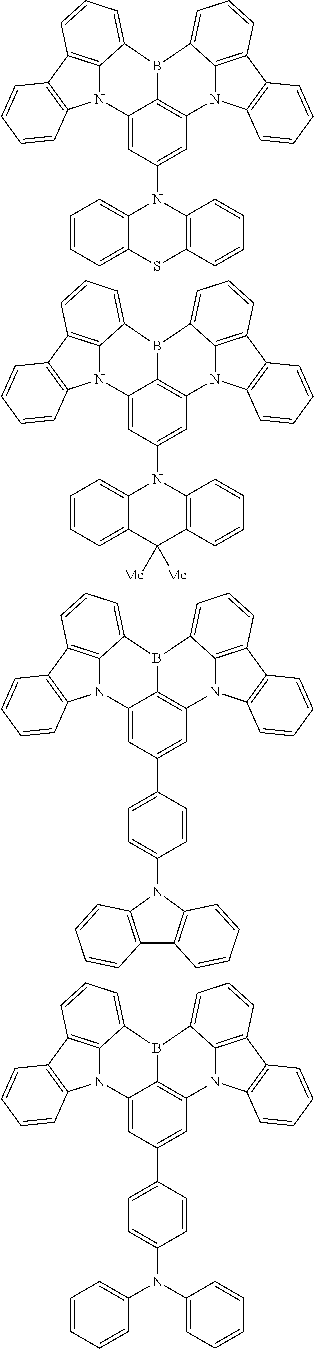

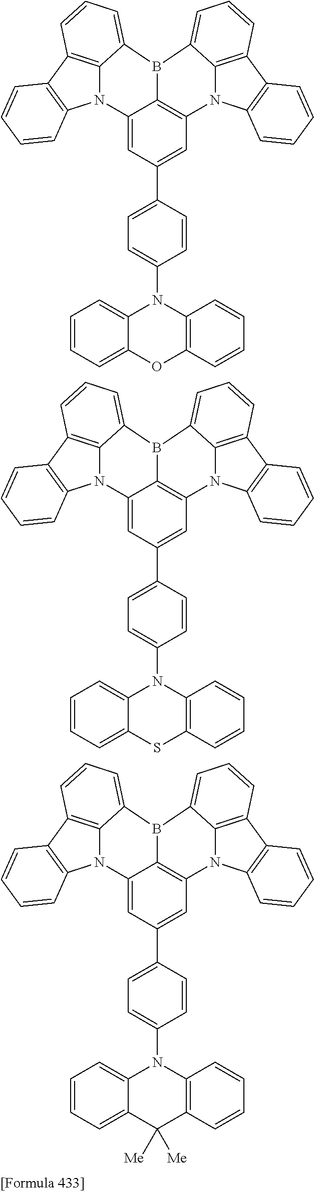

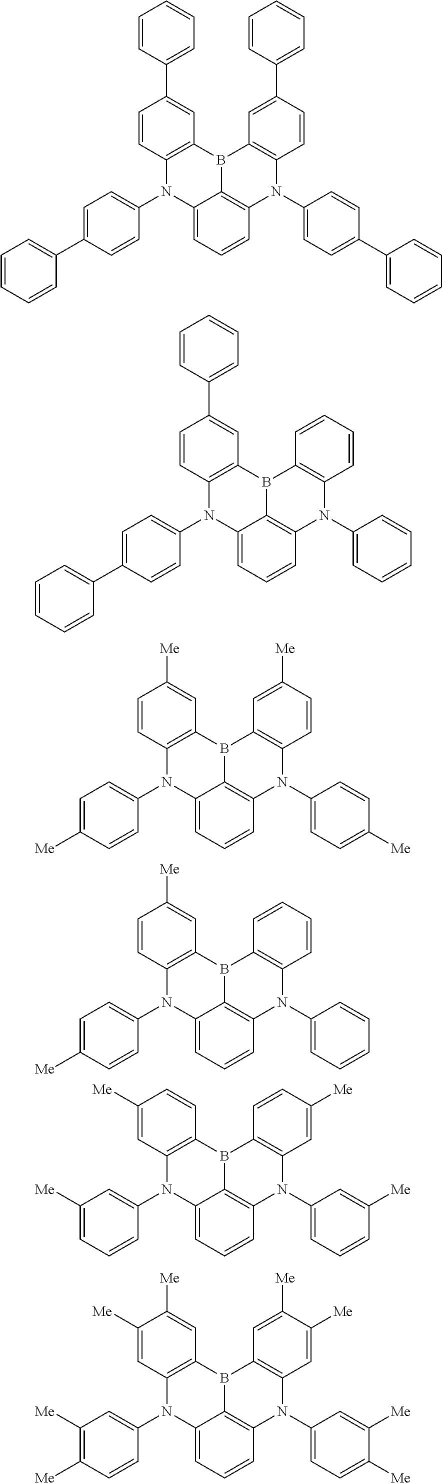

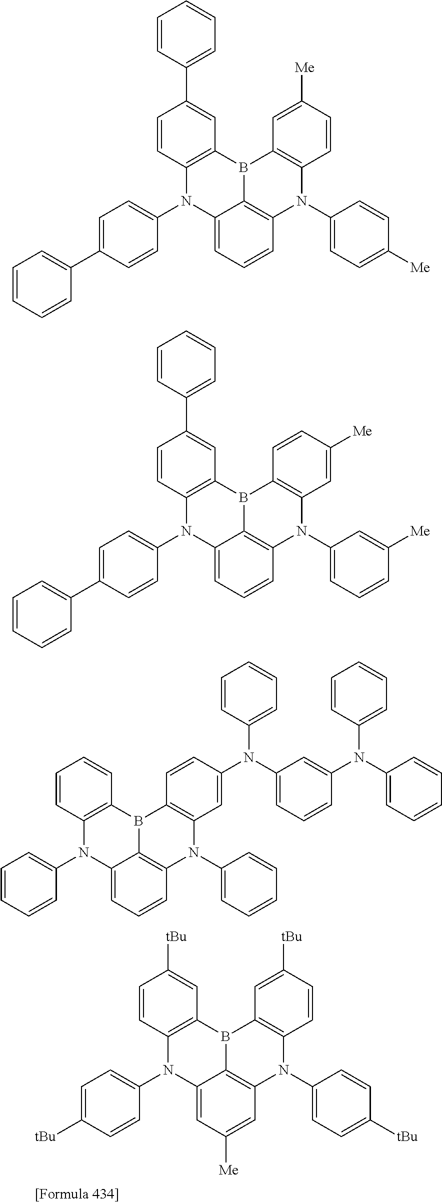

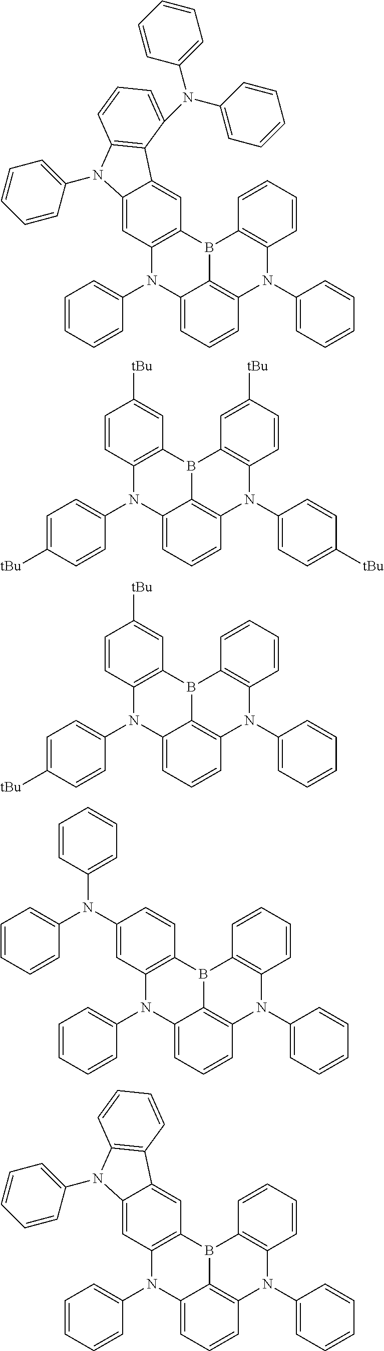

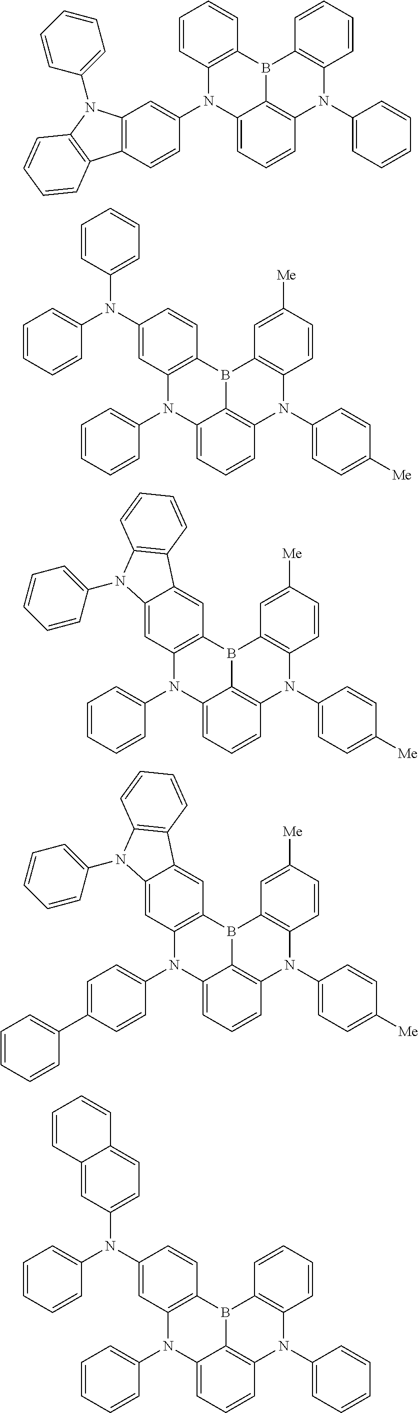

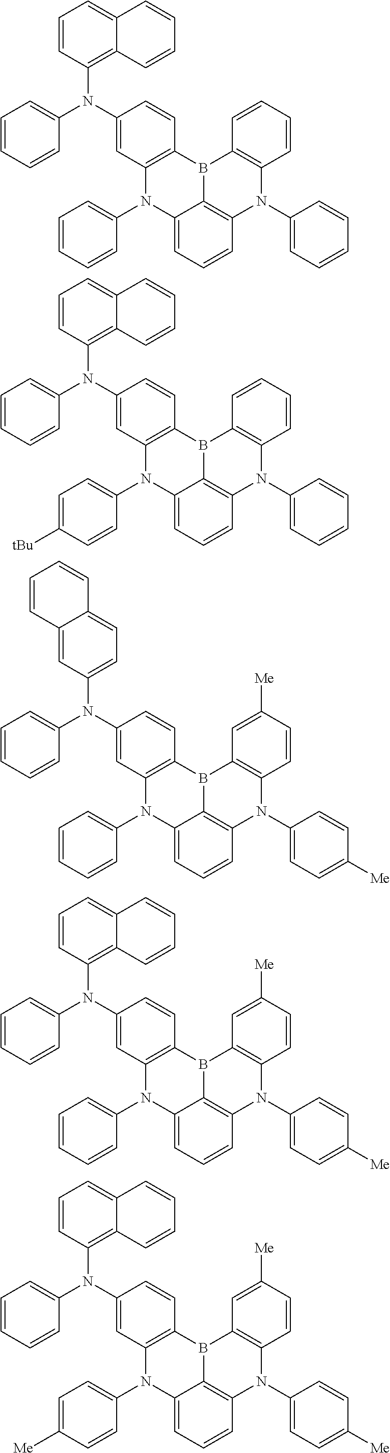

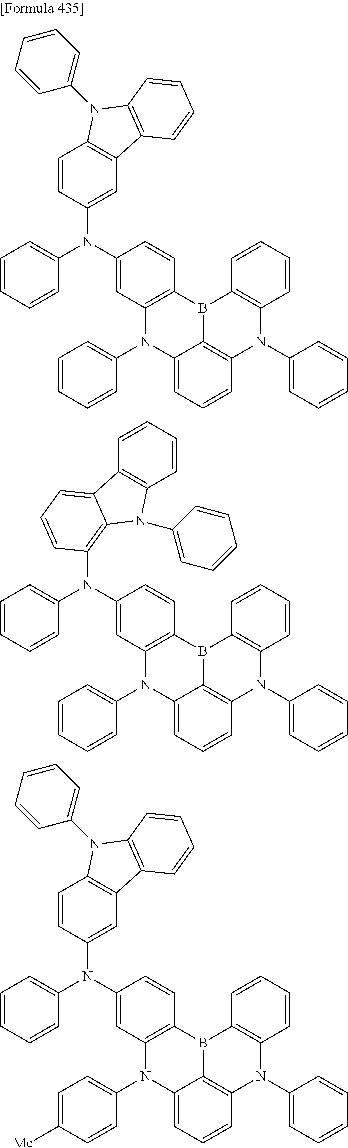

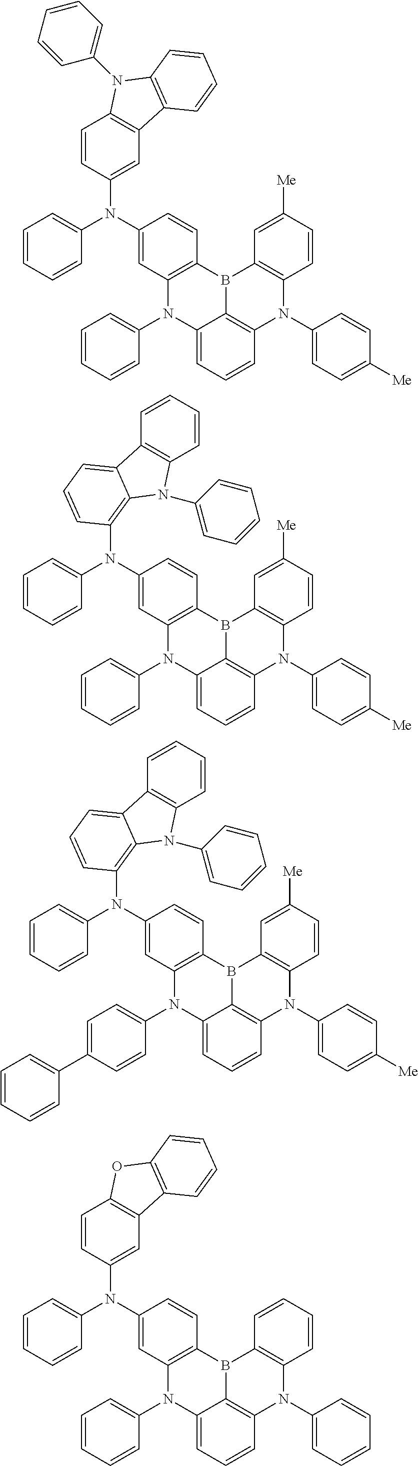

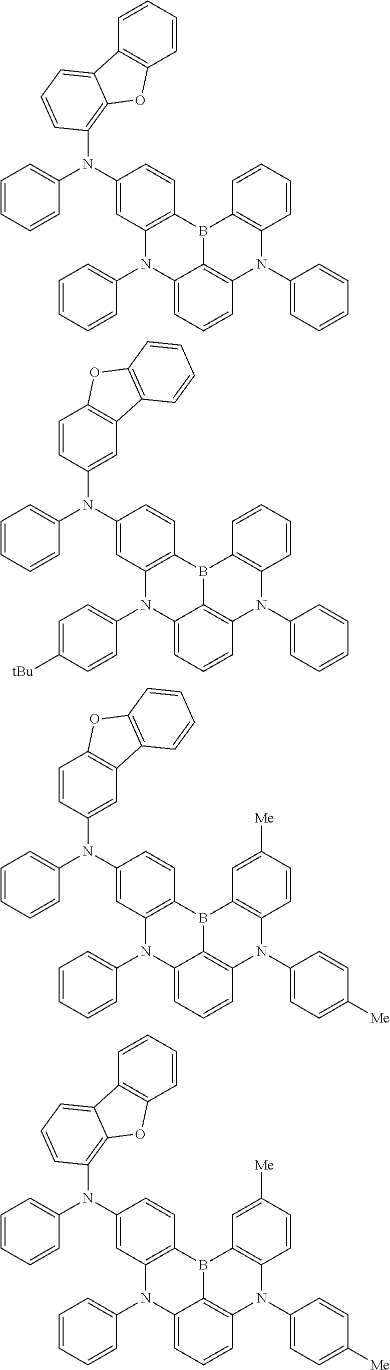

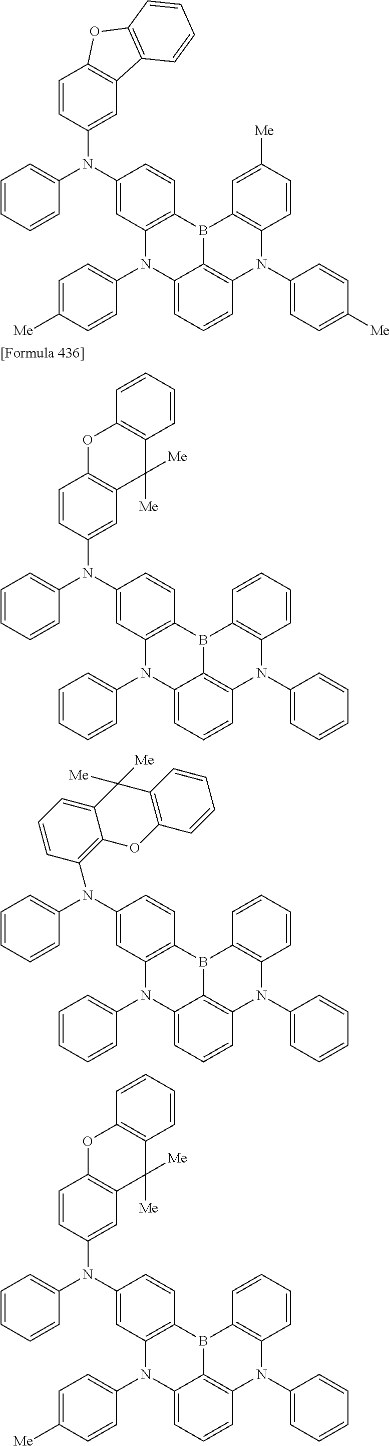

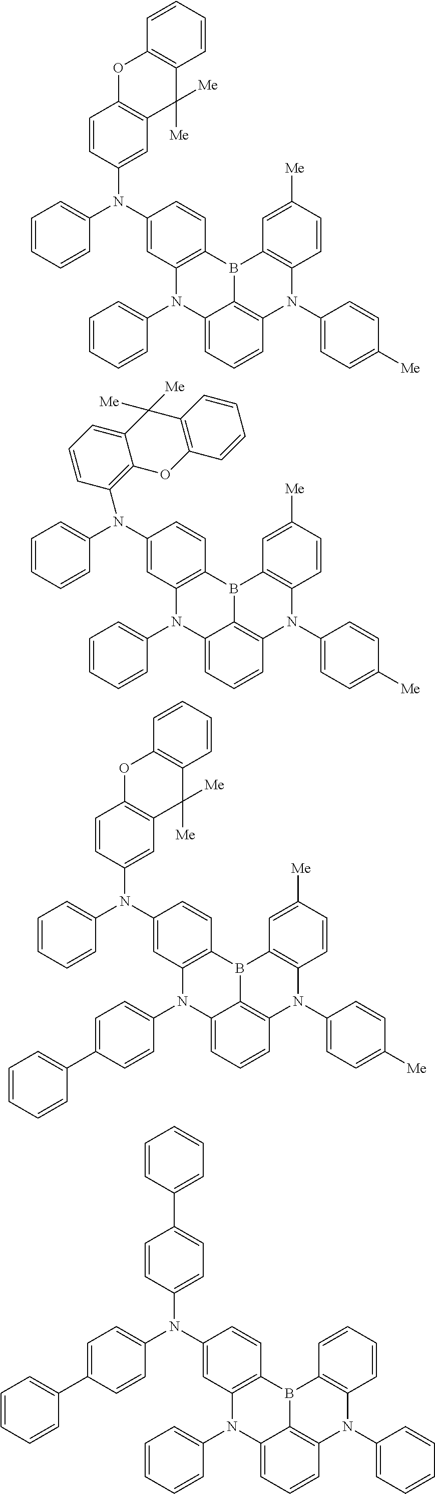

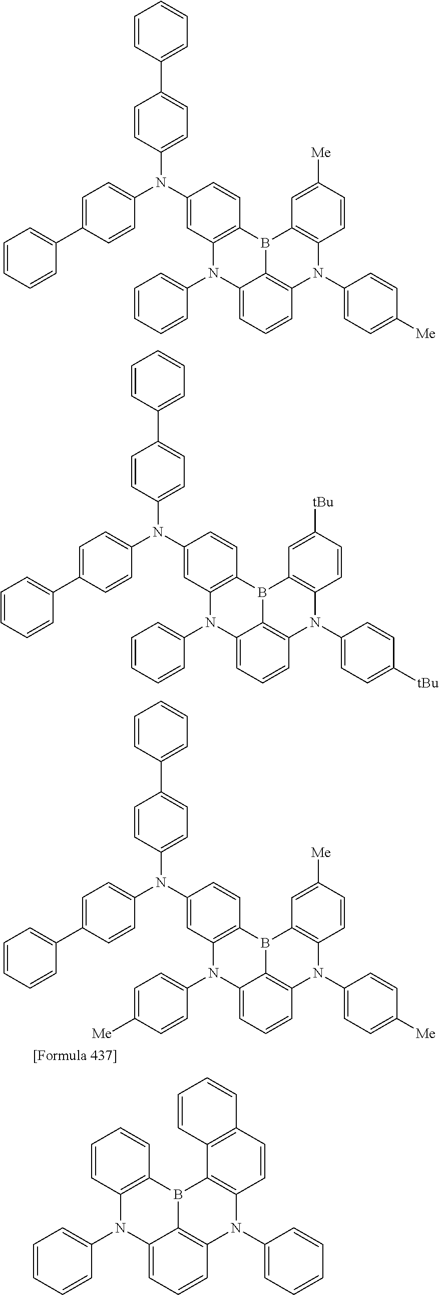

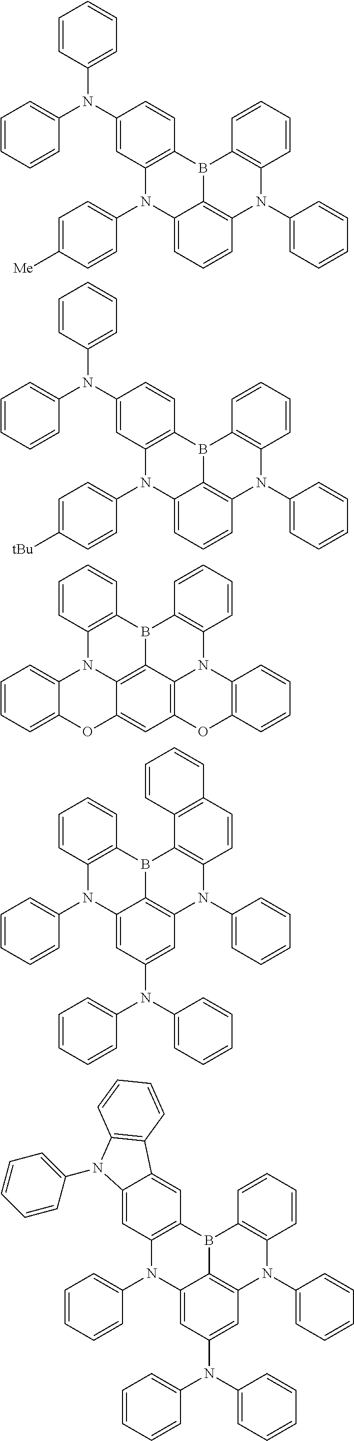

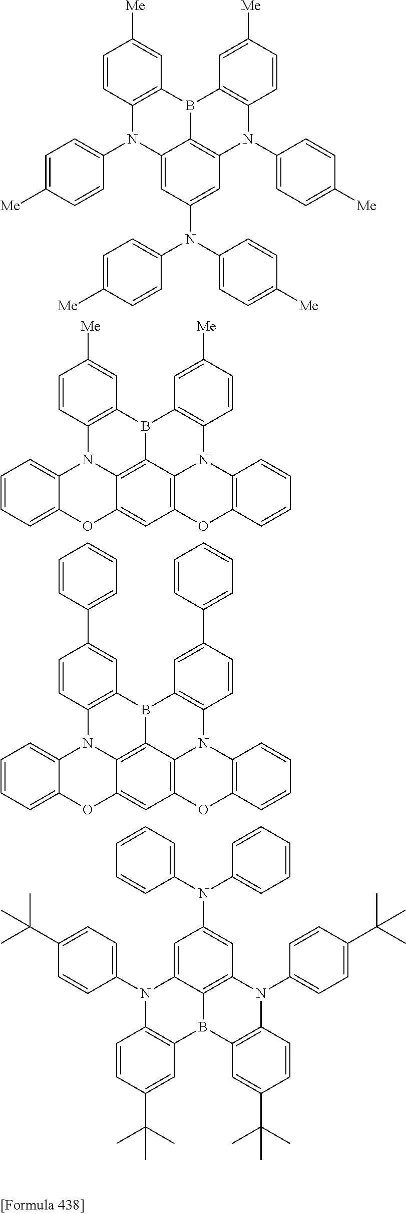

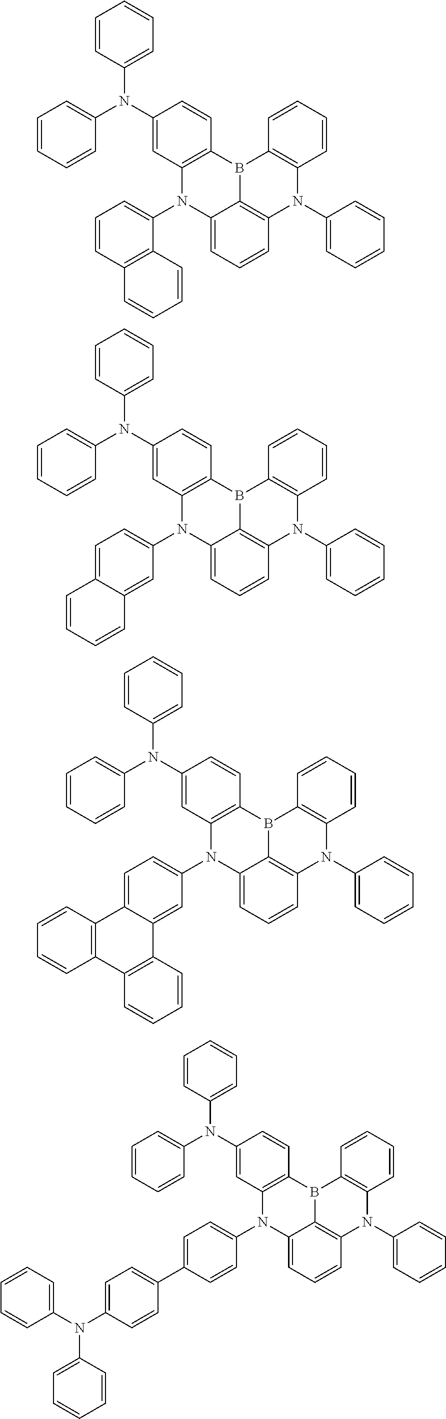

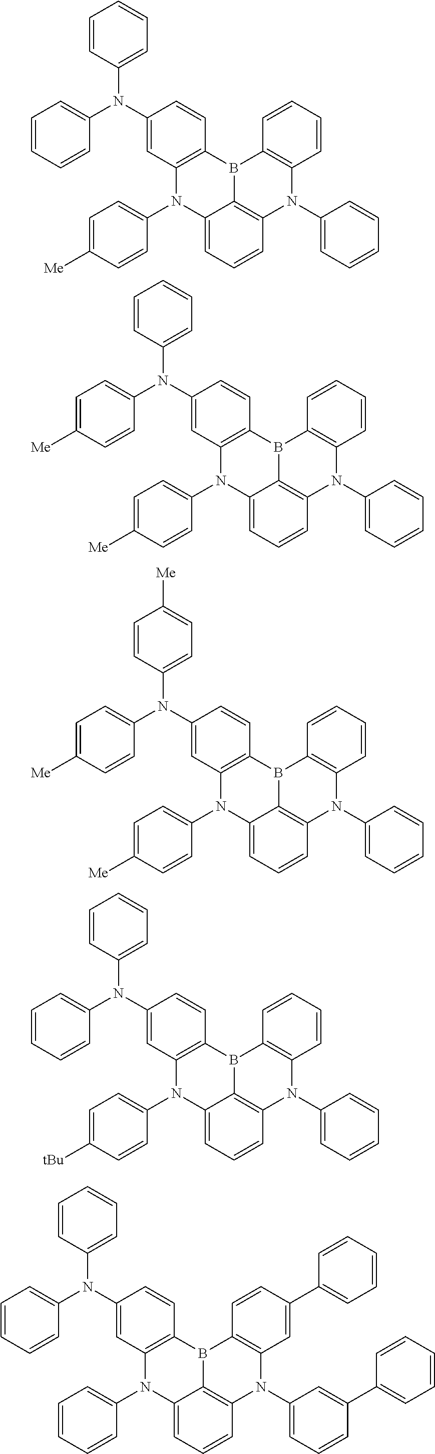

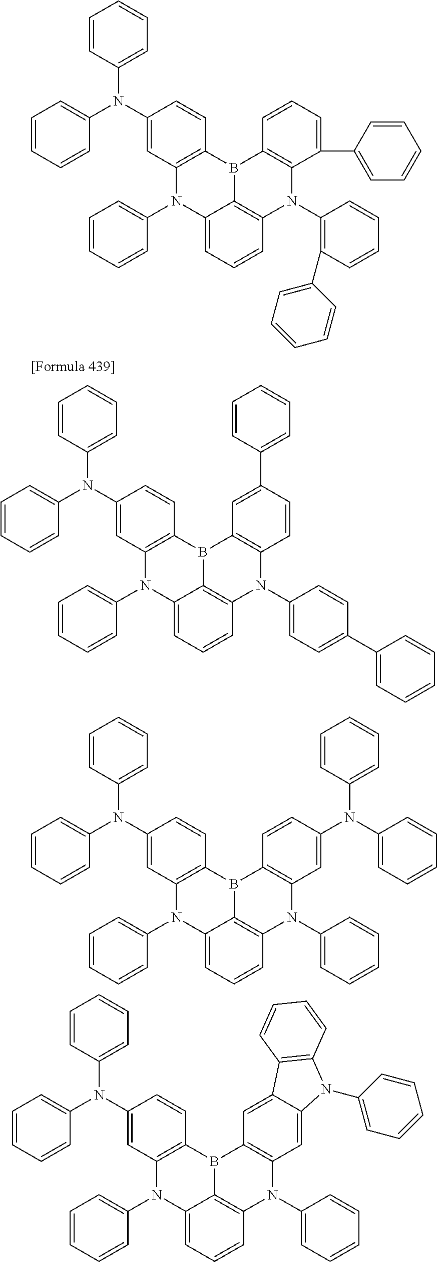

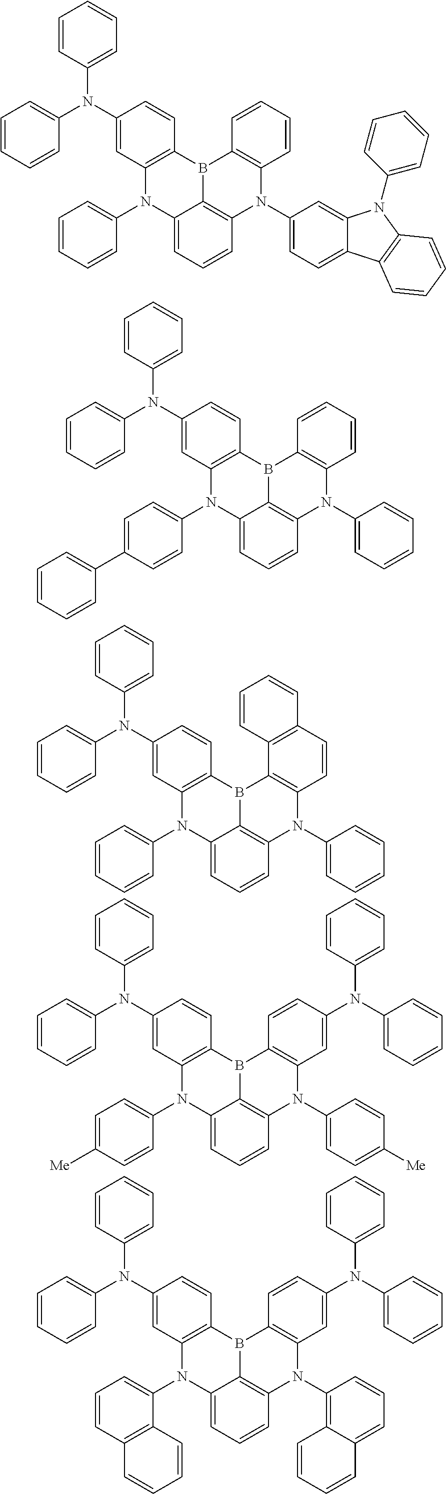

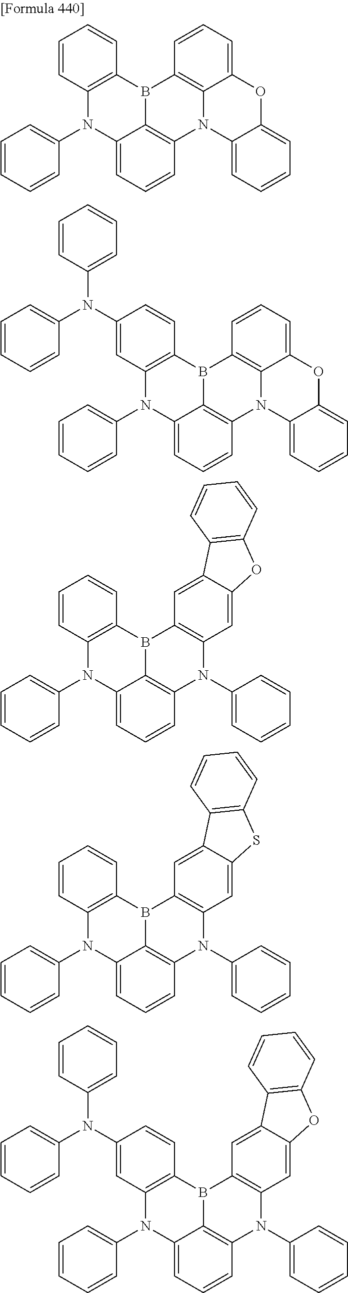

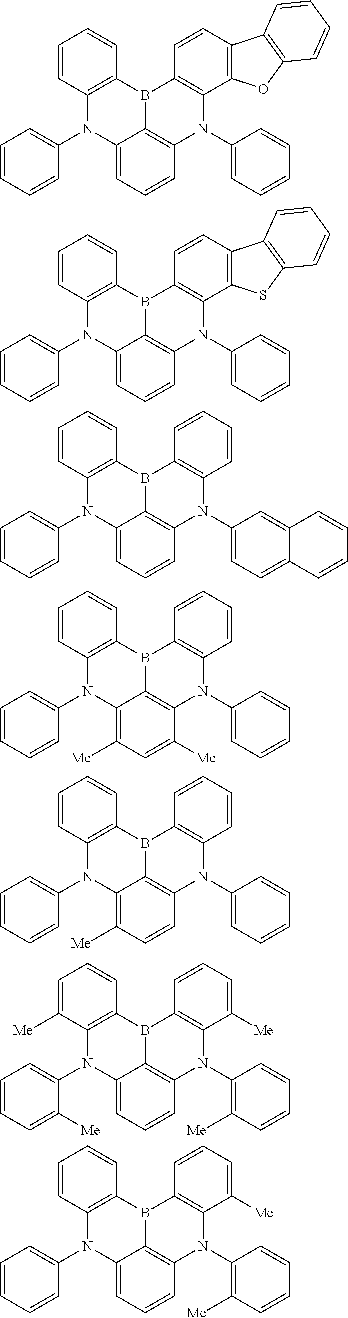

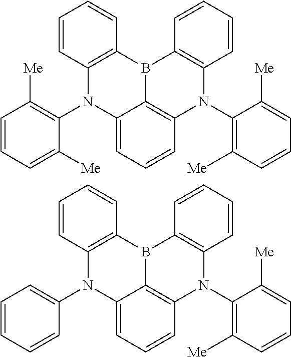

Compound Represented by Formula (7)

A compound represented by the formula (7) will be described below.

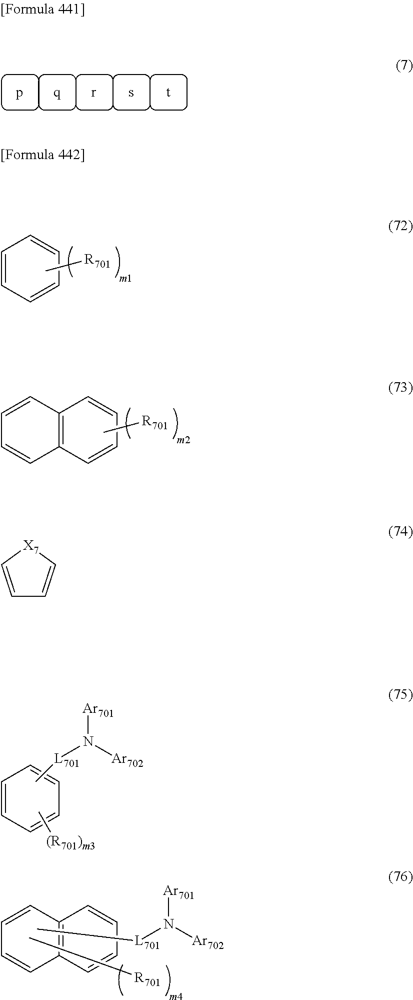

In the formula (7): r ring is a ring represented by the formula (72) or the formula (73), the r ring being fused with adjacent ring(s) at any position(s);
q ring and s ring are each independently a ring represented by the formula (74) and fused with adjacent ring(s) at any position(s);
p ring and t ring are each independently a structure represented by the formula (75) or the formula (76) and fused with adjacent ring(s) at any position(s);
X₇ is an oxygen atom, a sulfur atom, or NR₇₀₂;
when a plurality of R₇₀₁ are present, adjacent ones of the plurality of R₇₀₁ are mutually bonded to form a substituted or unsubstituted monocyclic ring, mutually bonded to form a substituted or unsubstituted fused ring, or not mutually bonded;
R₇₀₁ and R₇₀₂ forming neither the substituted or unsubstituted monocyclic ring nor the substituted or unsubstituted fused ring are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group Represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
Ar₇₀₁ and Ar₇₀₂ are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
L₇₀₁ is a substituted or unsubstituted alkylene group having 1 to 50 carbon atoms, a substituted or unsubstituted alkenylene group having 2 to 50 carbon atoms, a substituted or unsubstituted alkynylene group having 2 to 50 carbon atoms, a substituted or unsubstituted cycloalkylene group having 3 to 50 ring carbon atoms, a substituted or unsubstituted arylene group having 6 to 50 ring carbon atoms, or a substituted or unsubstituted divalent heterocyclic group having 5 to 50 ring atoms;
m1 is 0, 1, or 2;
m2 is 0, 1, 2, 3, or 4;
m3 is each independently 0, 1, 2, 3 or 3;
m4 is each independently 0, 1, 2, 3, 4, or 5;
when a plurality of R₇₀₁ are present, the plurality of R₇₀₁ are mutually the same or different;
when a plurality of X₇ are present, the plurality of X₇ are mutually the same or different;
when a plurality of R₇₀₂ are present, the plurality of R₇₀₂ are mutually the same or different;
when a plurality of Ar₇₀₁ are present, the plurality of Ar₇₀₁ are mutually the same or different;
when a plurality of Ar₇₀₂ are present, the plurality of Ar₇₀₂ are mutually the same or different; and
when a plurality of L₇₀₁ are present, the plurality of L₇₀₁ are mutually the same or different.

In the formula (7), each of the p ring, q ring, r ring, s ring, and t ring is fused with an adjacent ring(s) sharing two carbon atoms. The fused position and orientation are not limited but may be defined as required.

In an exemplary embodiment, in the formula (72) or the formula (73) representing the r ring, m1=0 or m2=0 is satisfied.

In an exemplary embodiment, a compound represented by the formula (7) is represented by any one of formulae (71-1) to (71-6) below.

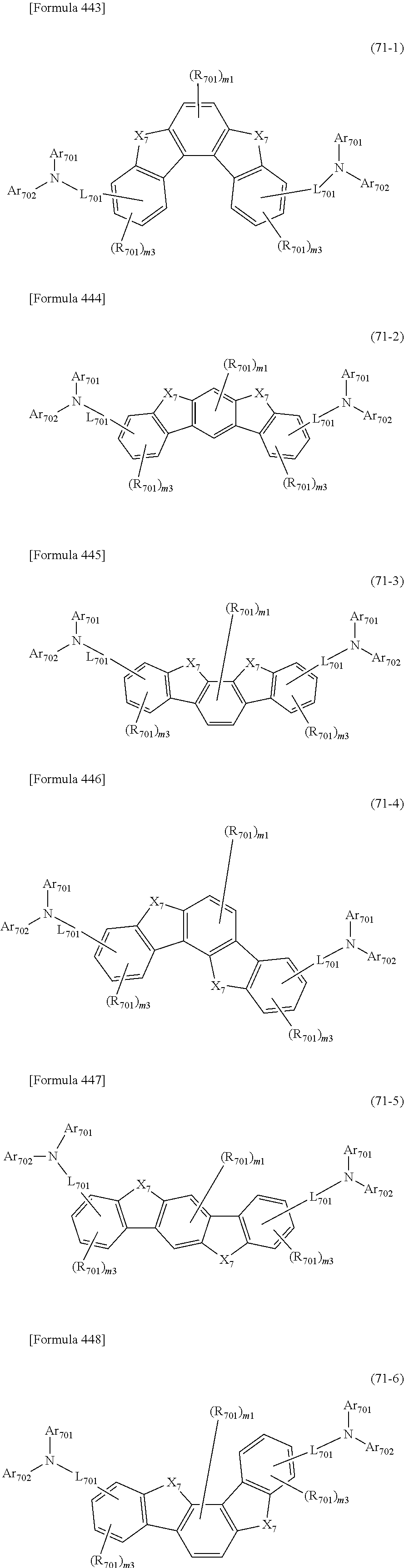

In the formulae (71-1) to (71-6), R₇₀₁, X₇, Ar₇₀₁, Ar₇₀₂, L₇₀₁, m1 and m3 respectively represent the same as R₇₀₁, X₇, Ar₇₀₁, Ar₇₀₂, L₇₀₁, m1 and m3 in the formula (7).

In an exemplary embodiment, a compound represented by the formula (7) is represented by any one of formulae (71-11) to (71-13) below.

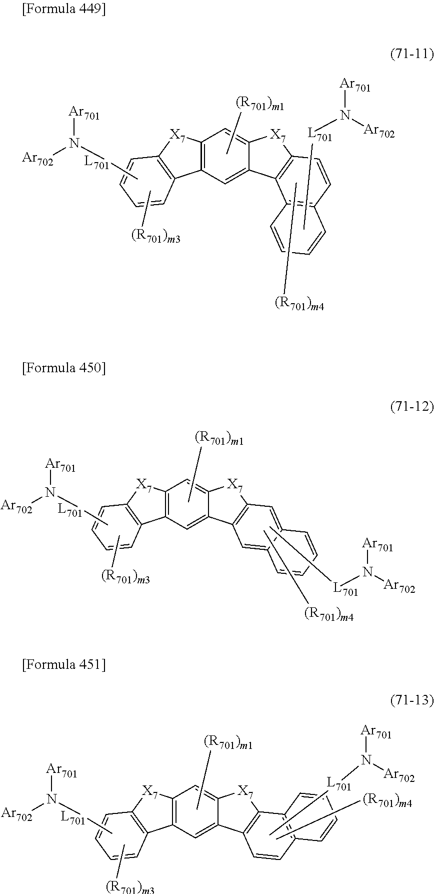

In the formulae (71-11) to (71-13), R₇₀₁, X₇, Ar₇₀₁, Ar₇₀₂, L₇₀₁, m1, m3 and m4 respectively represent the same as R₇₀₁, X₇, Ar₇₀₁, Ar₇₀₂, L₇₀₁, m1, m3 and m4 in the formula (7).

In an exemplary embodiment, a compound represented by the formula (7) is represented by any one of formulae (71-21) to (71-25) below.

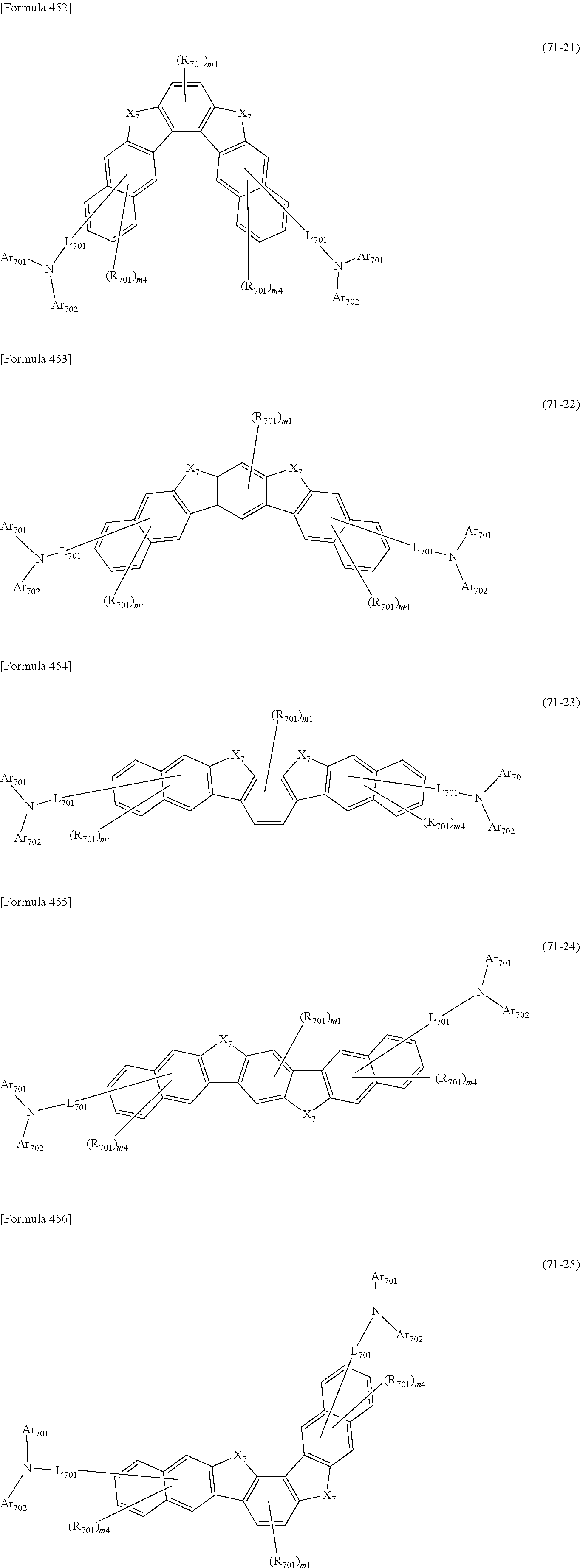

In the formulae (71-21) to (71-25), R₇₀₁, X₇, Ar₇₀₁, Ar₇₀₂, L₇₀₁, m1 and m4 respectively represent the same as R₇₀₁, X₇, Ar₇₀₁, Ar₇₀₂, L₇₀₁, m1 and m4 in the formula (7).

In an exemplary embodiment, a compound represented by the formula (7) is represented by any one of formulae (71-31) to (71-33) below.

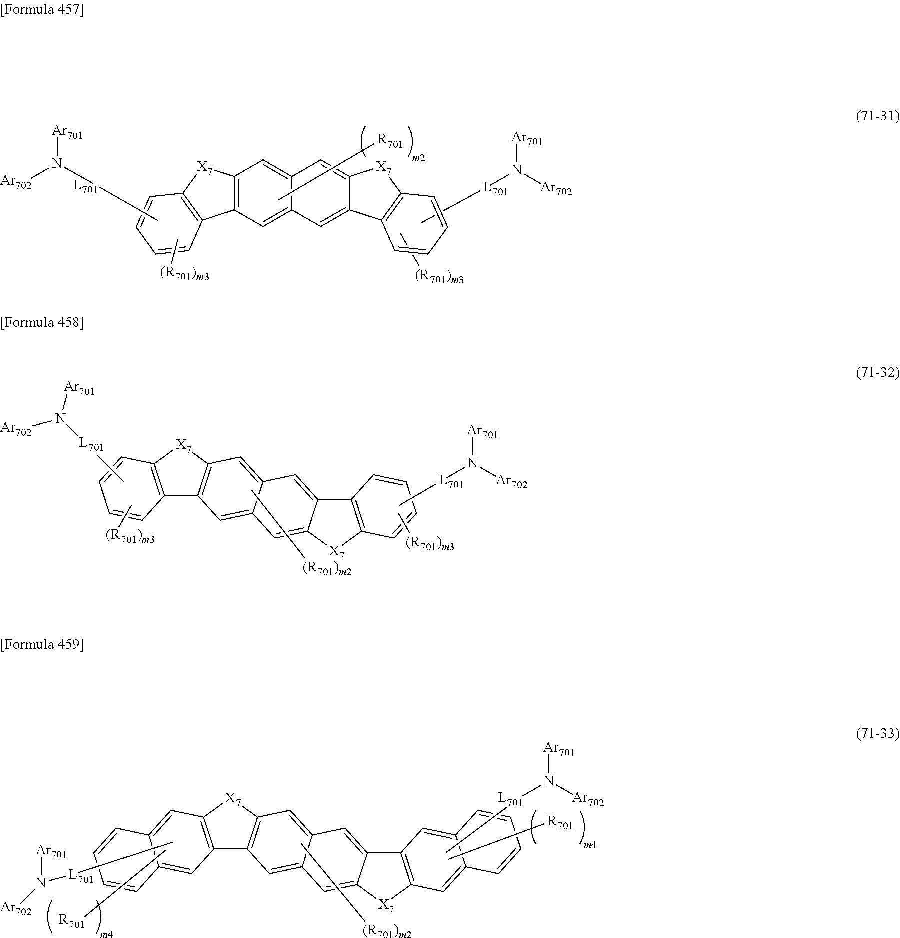

In the formulae (71-31) to (71-33), R₇₀₁, X₇, Ar₇₀₁, Ar₇₀₂, L₇₀₁, and m2 to m4 respectively represent the same as R₇₀₁, X₇, Ar₇₀₁, Ar₇₀₂, L₇₀₁, and m2 to m4 in the formula (7).

In an exemplary embodiment, Ar₇₀₁ and Ar₇₀₂ are each independently a substituted or unsubstituted aryl group having 6 to 50 ring carbon atoms.

In an exemplary embodiment, one of Ar₇₀₁ and Ar₇₀₂ is a substituted or unsubstituted aryl group having 6 to 50 ring carbon atoms, and the other of Ar₇₀₁ and Ar₇₀₂ is a substituted or unsubstituted heterocyclic group having 5 to 50 ring atoms.

Specific examples of a compound represented by the formula (7) include compounds shown below.

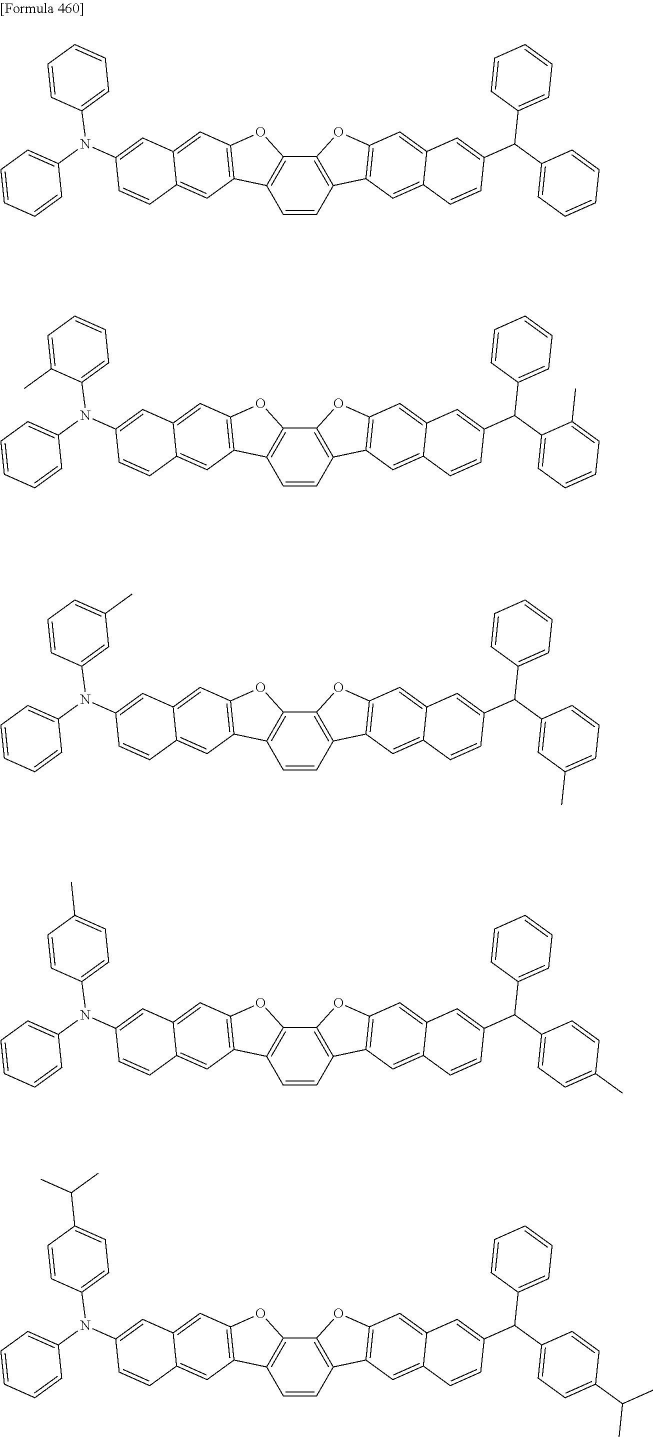 [SMILES: c1ccc(C(c2ccccc2)c2ccc3cc4c(cc3c2)oc2c4ccc3c4cc5ccc(N(c6ccccc6)c6ccccc6)cc5cc4oc32)cc1 ; Cc1ccccc1C(c1ccccc1)c1ccc2cc3c(cc2c1)oc1c3ccc2c3cc4ccc(N(c5ccccc5)c5ccccc5C)cc4cc3oc21 ; Cc1cccc(C(c2ccccc2)c2ccc3cc4c(cc3c2)oc2c4ccc3c4cc5ccc(N(c6ccccc6)c6cccc(C)c6)cc5cc4oc32)c1 ; Cc1ccc(C(c2ccccc2)c2ccc3cc4c(cc3c2)oc2c4ccc3c4cc5ccc(N(c6ccccc6)c6ccc(C)cc6)cc5cc4oc32)cc1 ; CC(C)c1ccc(C(c2ccccc2)c2ccc3cc4c(cc3c2)oc2c4ccc3c4cc5ccc(N(c6ccccc6)c6ccc(C(C)C)cc6)cc5cc4oc32)cc1]

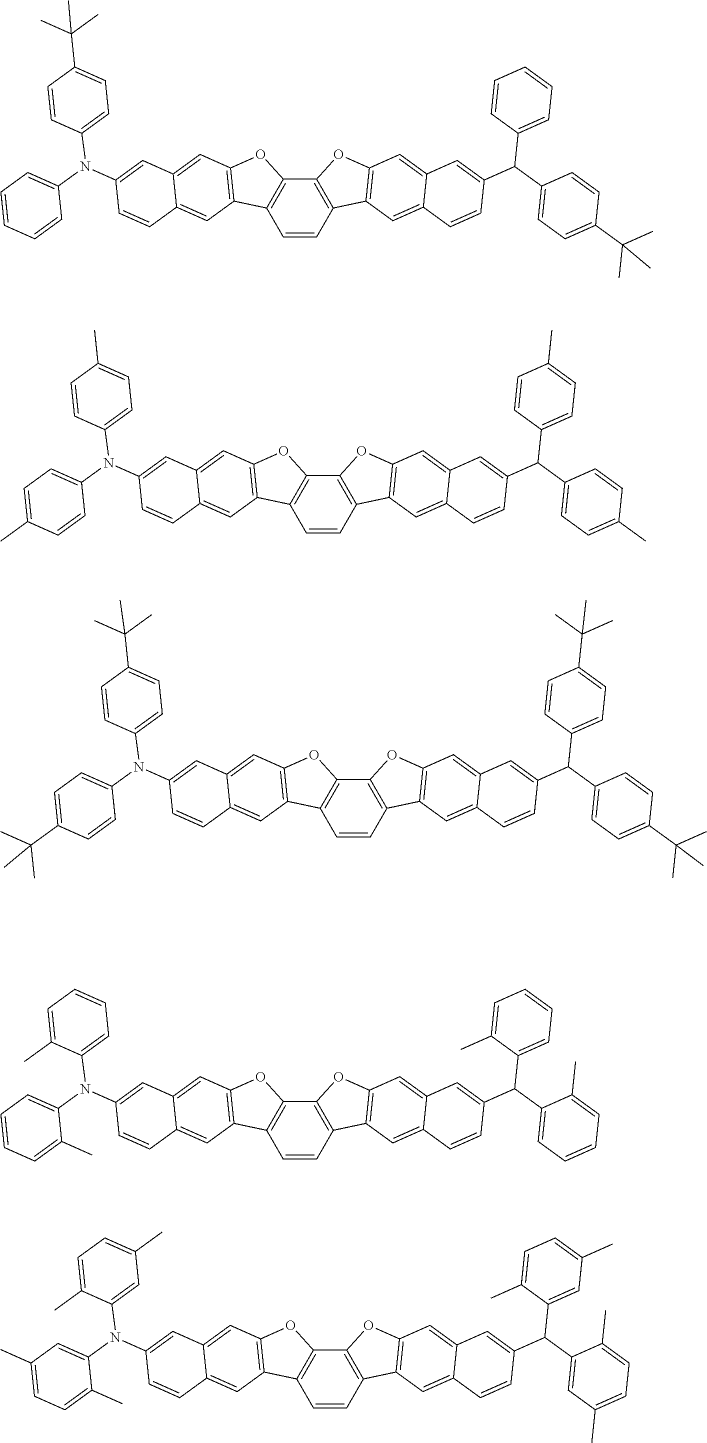

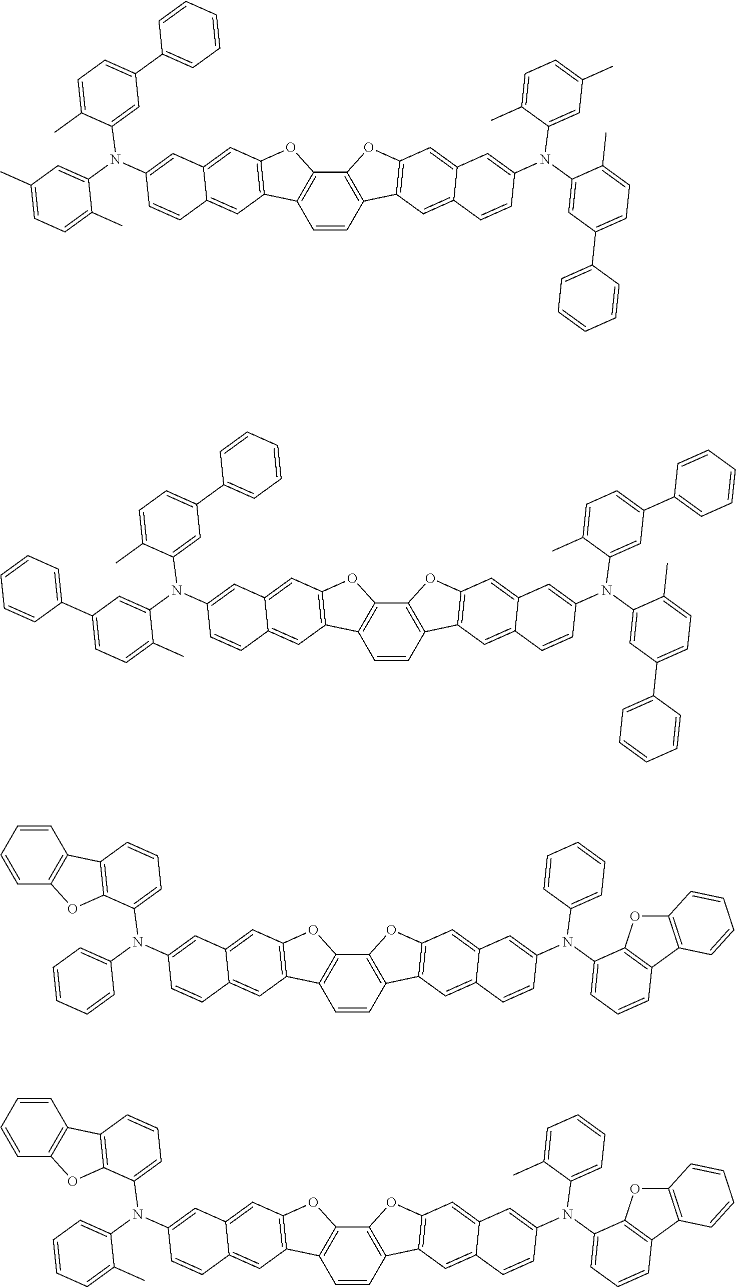 [SMILES: Cc1ccc(C)c(N(c2ccc3cc4c(cc3c2)oc2c4ccc3c4cc5ccc(N(c6cc(C)ccc6C)c6cc(-c7ccccc7)ccc6C)cc5cc4oc32)c2cc(-c3ccccc3)ccc2C)c1 ; Cc1ccc(-c2ccccc2)cc1N(c1ccc2cc3c(cc2c1)oc1c3ccc2c3cc4ccc(N(c5cc(-c6ccccc6)ccc5C)c5cc(-c6ccccc6)ccc5C)cc4cc3oc21)c1cc(-c2ccccc2)ccc1C ; c1ccc(N(c2ccc3cc4c(cc3c2)oc2c4ccc3c4cc5ccc(N(c6ccccc6)c6cccc7c6oc6ccccc67)cc5cc4oc32)c2cccc3c2oc2ccccc23)cc1 ; Cc1ccccc1N(c1ccc2cc3c(cc2c1)oc1c3ccc2c3cc4ccc(N(c5ccccc5C)c5cccc6c5oc5ccccc56)cc4cc3oc21)c1cccc2c1oc1ccccc12]

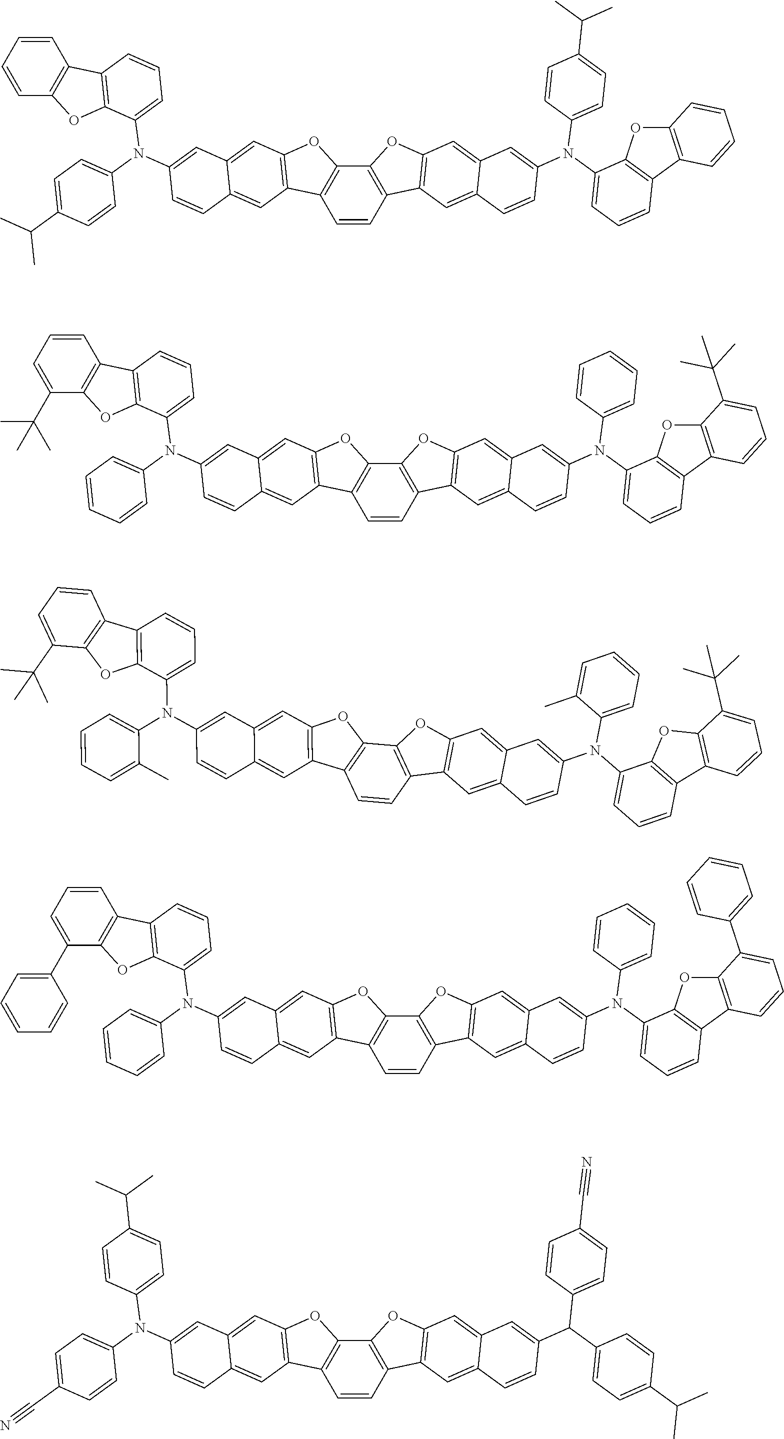

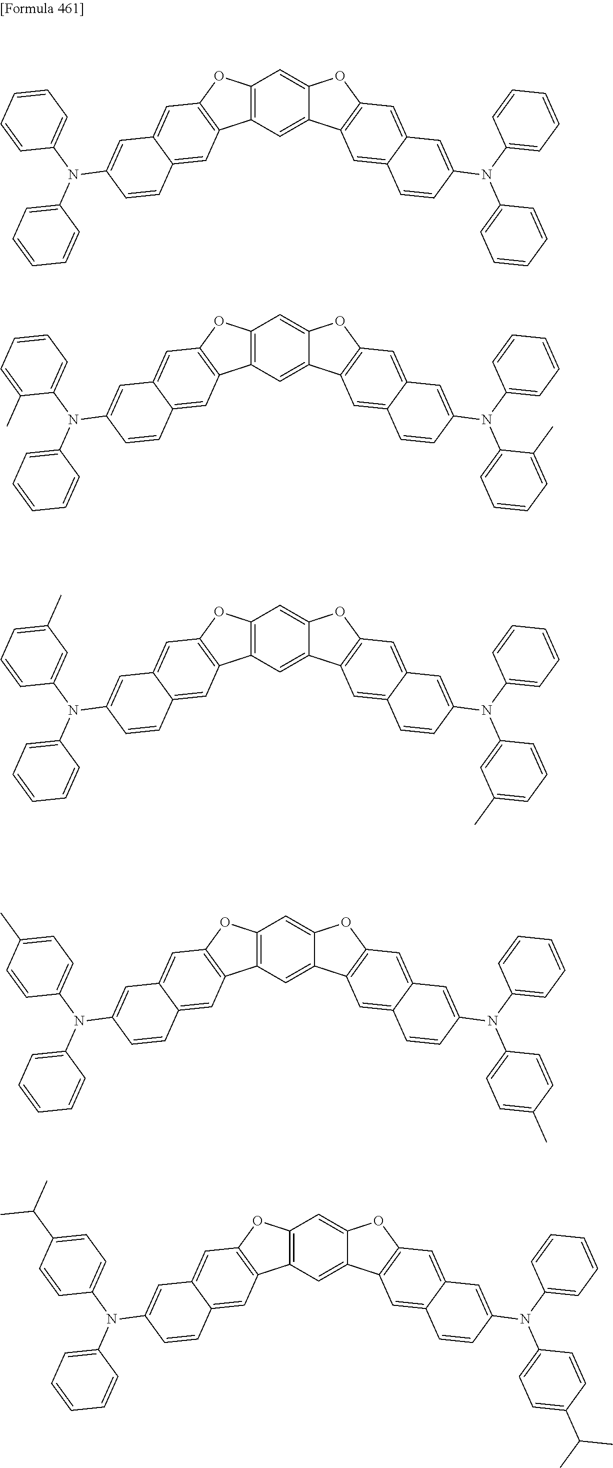 [SMILES: c1ccc(N(c2ccccc2)c2ccc3cc4c(cc3c2)oc2cc3oc5cc6cc(N(c7ccccc7)c7ccccc7)ccc6cc5c3cc24)cc1 ; Cc1ccccc1N(c1ccccc1)c1ccc2cc3c(cc2c1)oc1cc2oc4cc5cc(N(c6ccccc6)c6ccccc6C)ccc5cc4c2cc13 ; Cc1cccc(N(c2ccccc2)c2ccc3cc4c(cc3c2)oc2cc3oc5cc6cc(N(c7ccccc7)c7cccc(C)c7)ccc6cc5c3cc24)c1 ; Cc1ccc(N(c2ccccc2)c2ccc3cc4c(cc3c2)oc2cc3oc5cc6cc(N(c7ccccc7)c7ccc(C)cc7)ccc6cc5c3cc24)cc1 ; CC(C)c1ccc(N(c2ccccc2)c2ccc3cc4c(cc3c2)oc2cc3oc5cc6cc(N(c7ccccc7)c7ccc(C(C)C)cc7)ccc6cc5c3cc24)cc1]

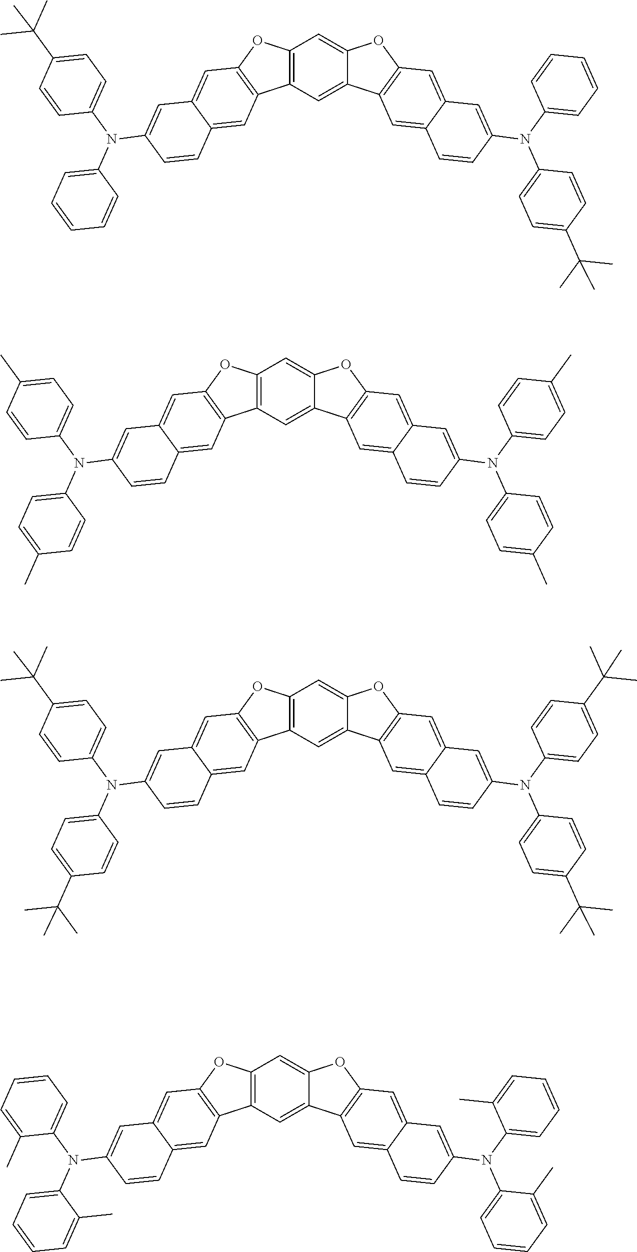

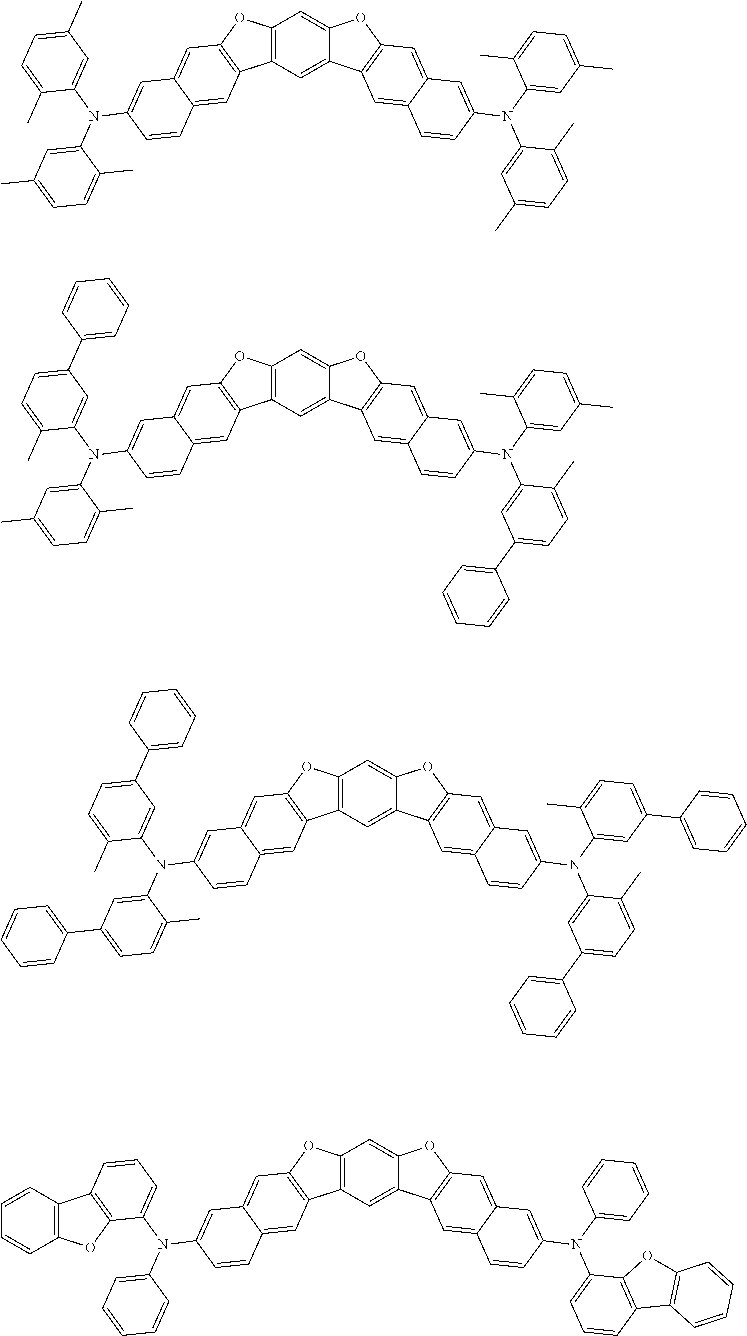

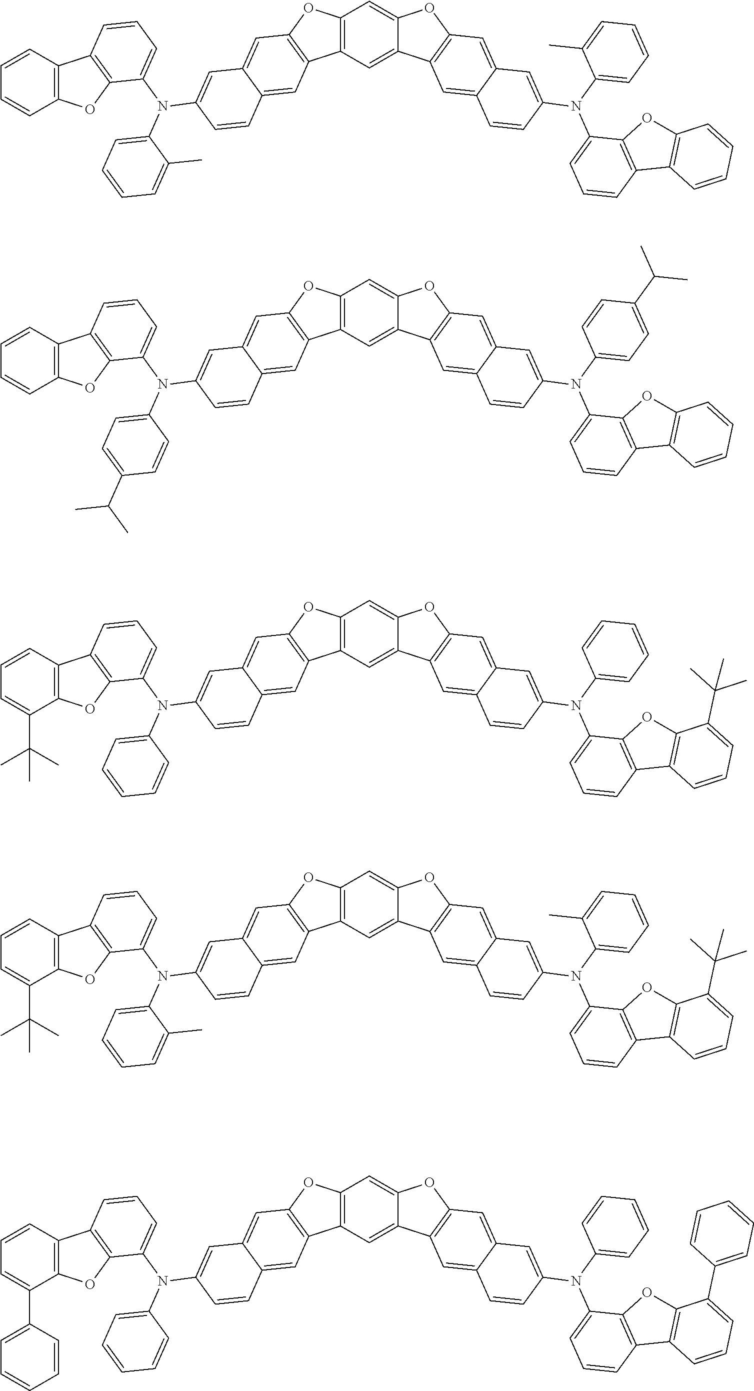

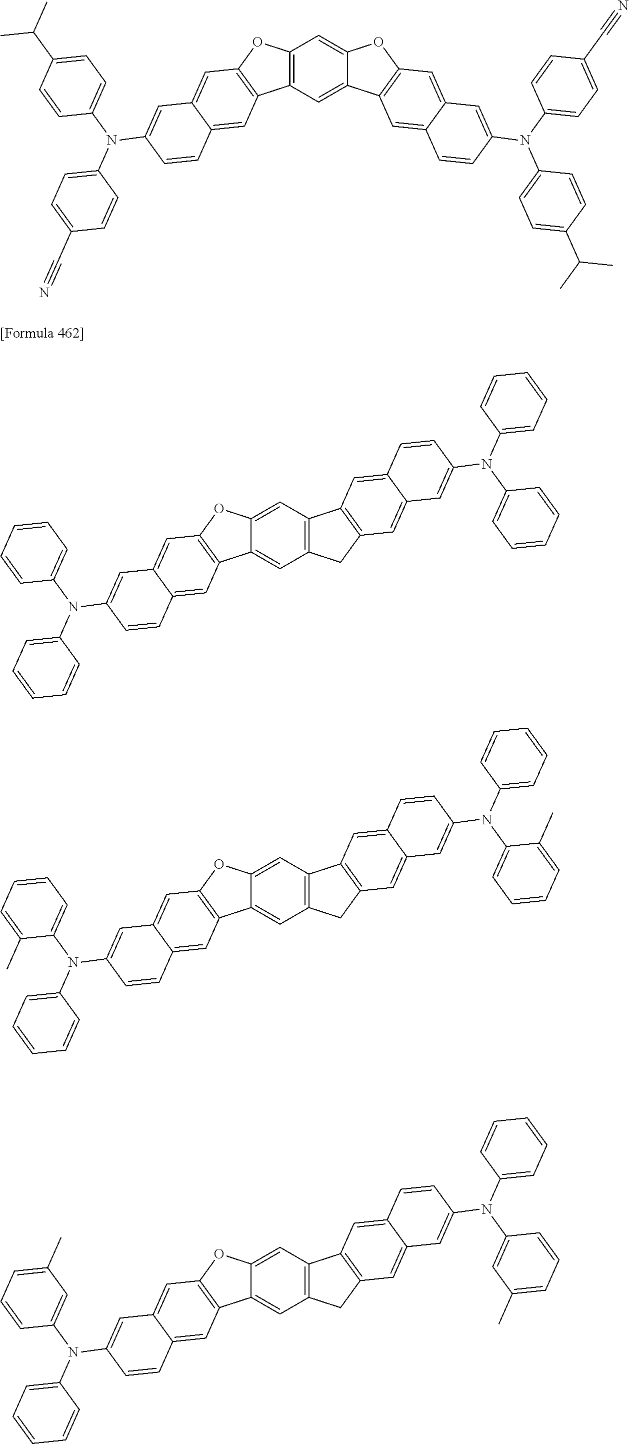 [SMILES: CC(C)c1ccc(N(c2ccc(C#N)cc2)c2ccc3cc4c(cc3c2)oc2cc3oc5cc6cc(N(c7ccc(C#N)cc7)c7ccc(C(C)C)cc7)ccc6cc5c3cc24)cc1 ; c1ccc(N(c2ccccc2)c2ccc3cc4c(cc3c2)Cc2cc3c(cc2-4)oc2cc4cc(N(c5ccccc5)c5ccccc5)ccc4cc23)cc1 ; Cc1ccccc1N(c1ccccc1)c1ccc2cc3c(cc2c1)Cc1cc2c(cc1-3)oc1cc3cc(N(c4ccccc4)c4ccccc4C)ccc3cc12 ; Cc1cccc(N(c2ccccc2)c2ccc3cc4c(cc3c2)Cc2cc3c(cc2-4)oc2cc4cc(N(c5ccccc5)c5cccc(C)c5)ccc4cc23)c1]

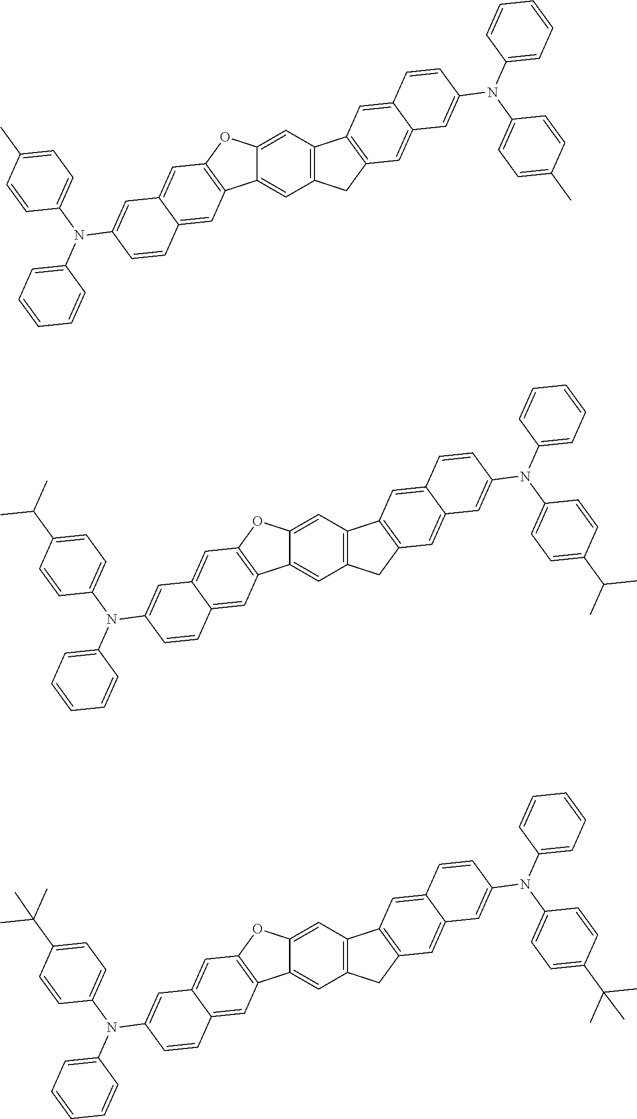

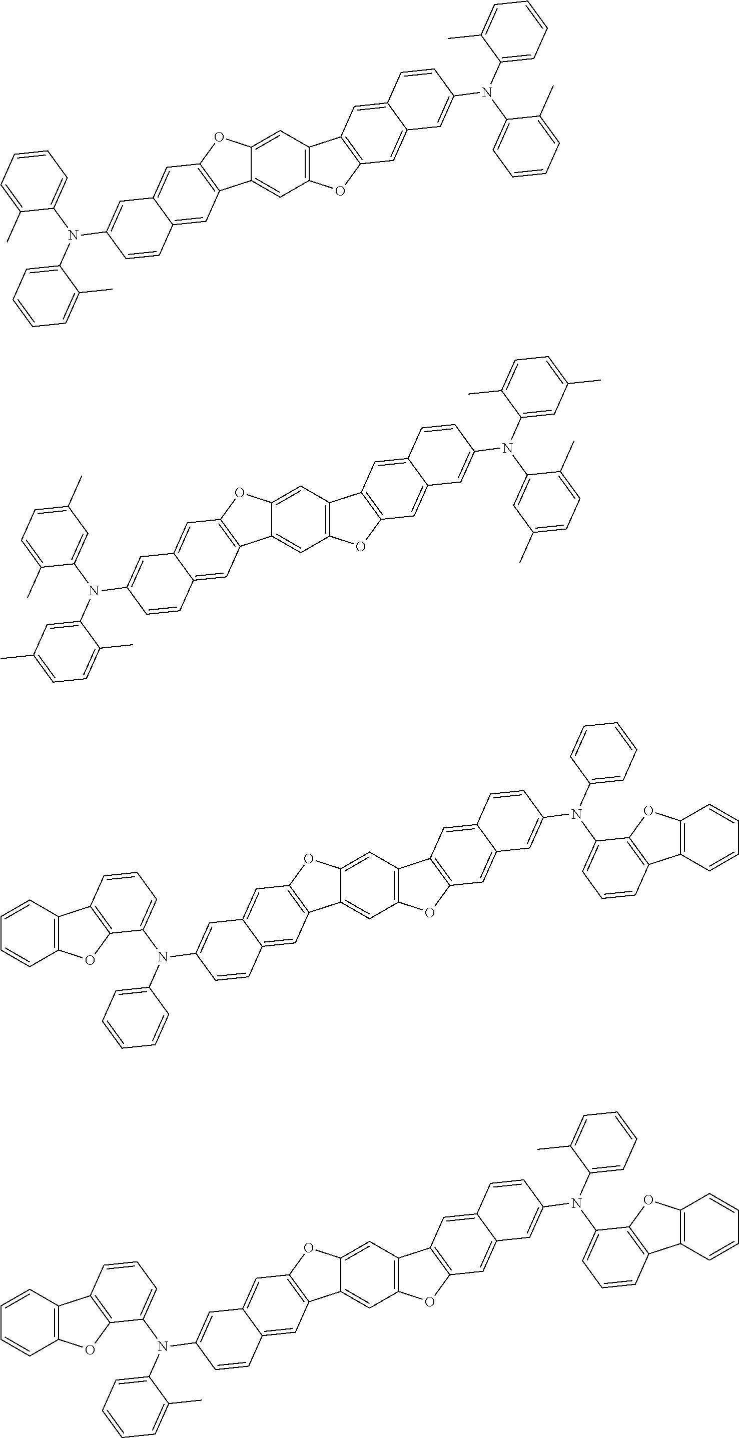

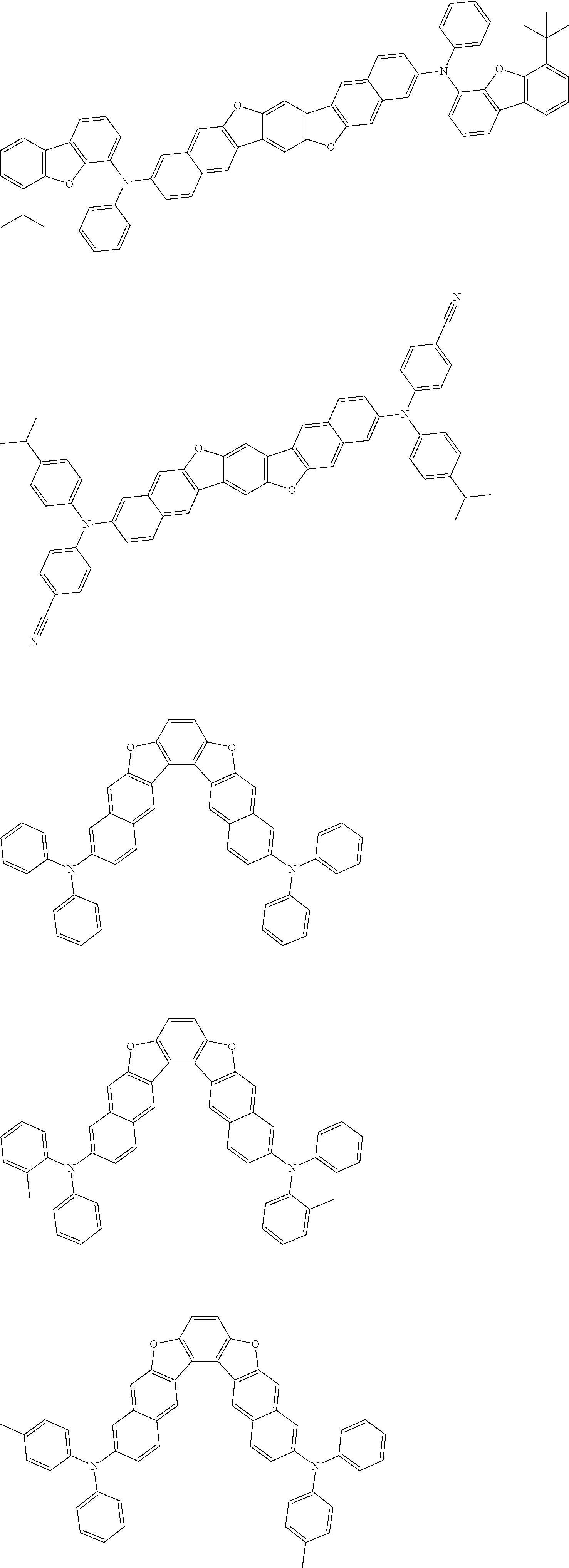

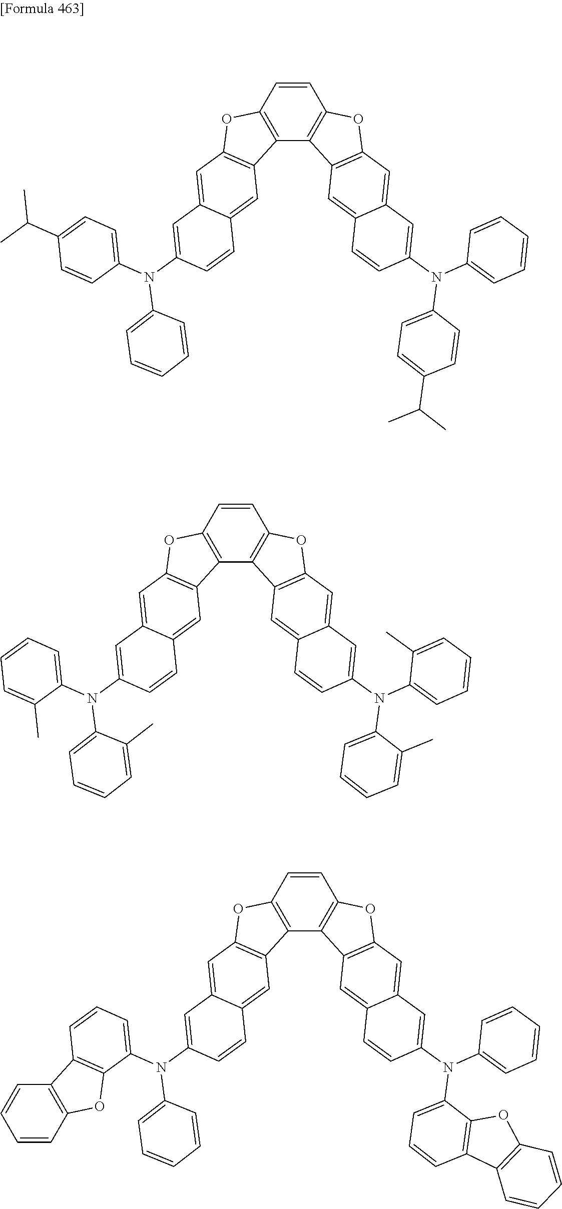 [SMILES: CC(C)c1ccc(N(c2ccccc2)c2ccc3cc4c(cc3c2)oc2ccc3oc5cc6cc(N(c7ccccc7)c7ccc(C(C)C)cc7)ccc6cc5c3c24)cc1 ; Cc1ccccc1N(c1ccc2cc3c(cc2c1)oc1ccc2oc4cc5cc(N(c6ccccc6C)c6ccccc6C)ccc5cc4c2c13)c1ccccc1C ; c1ccc(N(c2ccc3cc4c(cc3c2)oc2ccc3oc5cc6cc(N(c7ccccc7)c7cccc8c7oc7ccccc78)ccc6cc5c3c24)c2cccc3c2oc2ccccc23)cc1]

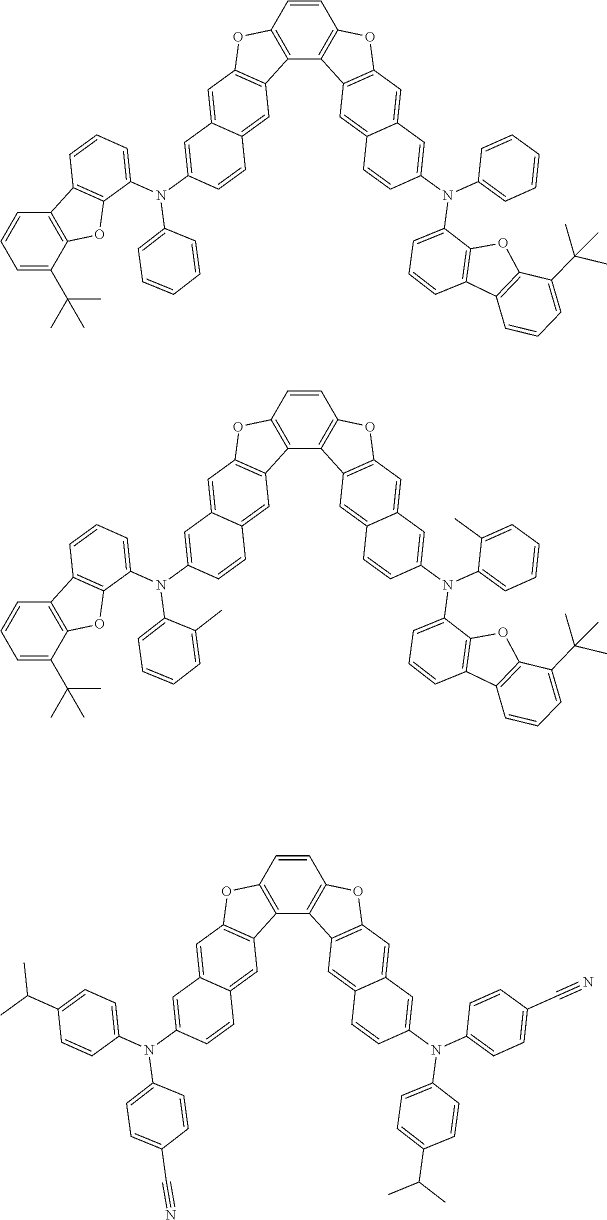

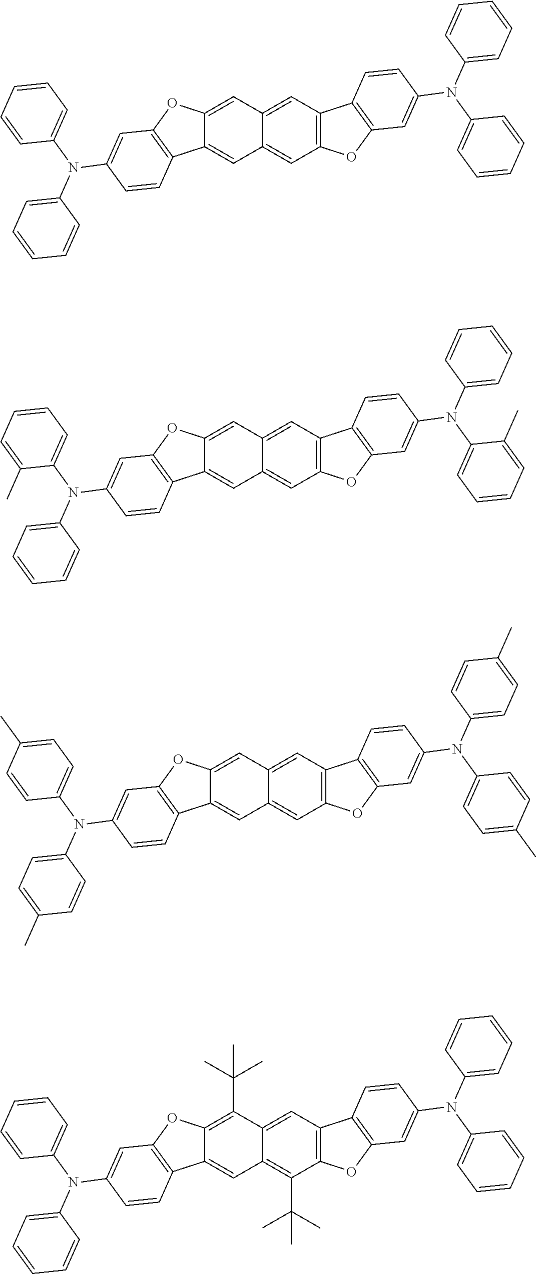

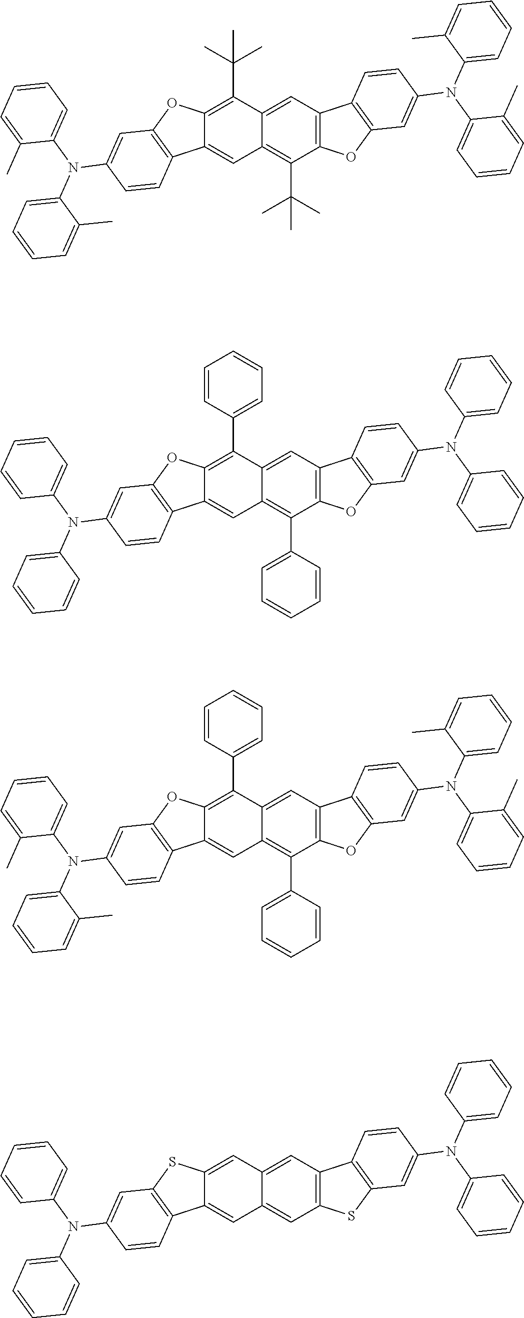 [SMILES: Cc1ccccc1N(c1ccc2c(c1)oc1c(C(C)(C)C)c3cc4c(oc5cc(N(c6ccccc6C)c6ccccc6C)ccc54)c(C(C)(C)C)c3cc12)c1ccccc1C ; c1ccc(-c2c3cc4c(oc5cc(N(c6ccccc6)c6ccccc6)ccc54)c(-c4ccccc4)c3cc3c2oc2cc(N(c4ccccc4)c4ccccc4)ccc23)cc1 ; Cc1ccccc1N(c1ccc2c(c1)oc1c(-c3ccccc3)c3cc4c(oc5cc(N(c6ccccc6C)c6ccccc6C)ccc54)c(-c4ccccc4)c3cc12)c1ccccc1C ; c1ccc(N(c2ccccc2)c2ccc3c(c2)sc2cc4cc5c(cc4cc23)sc2cc(N(c3ccccc3)c3ccccc3)ccc25)cc1]

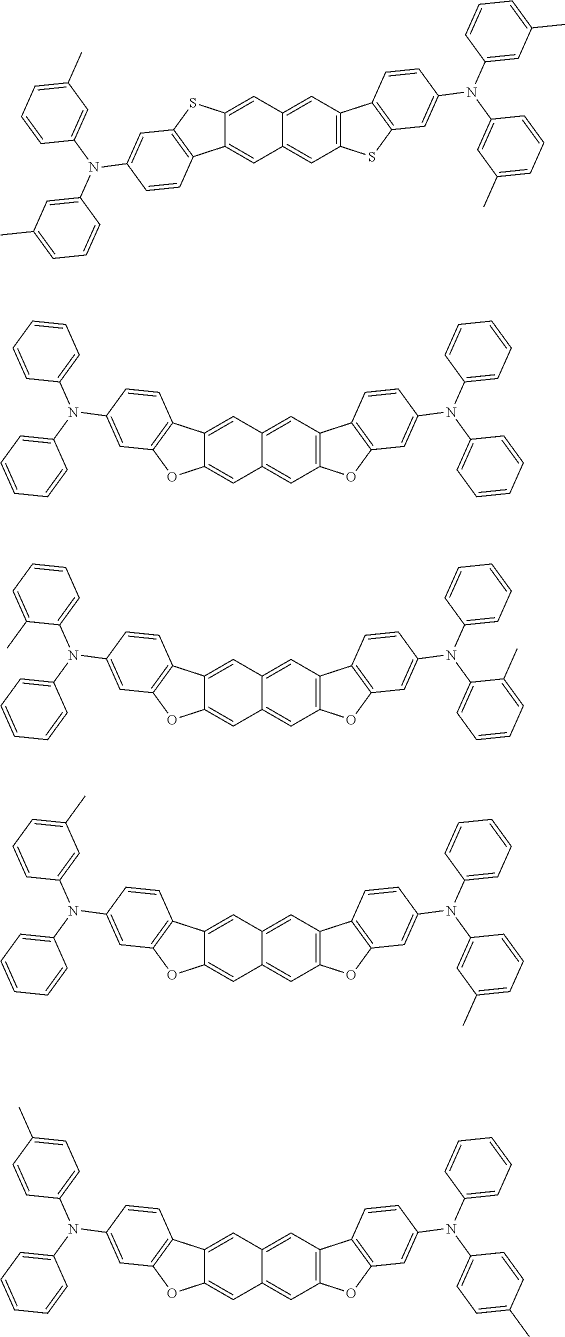

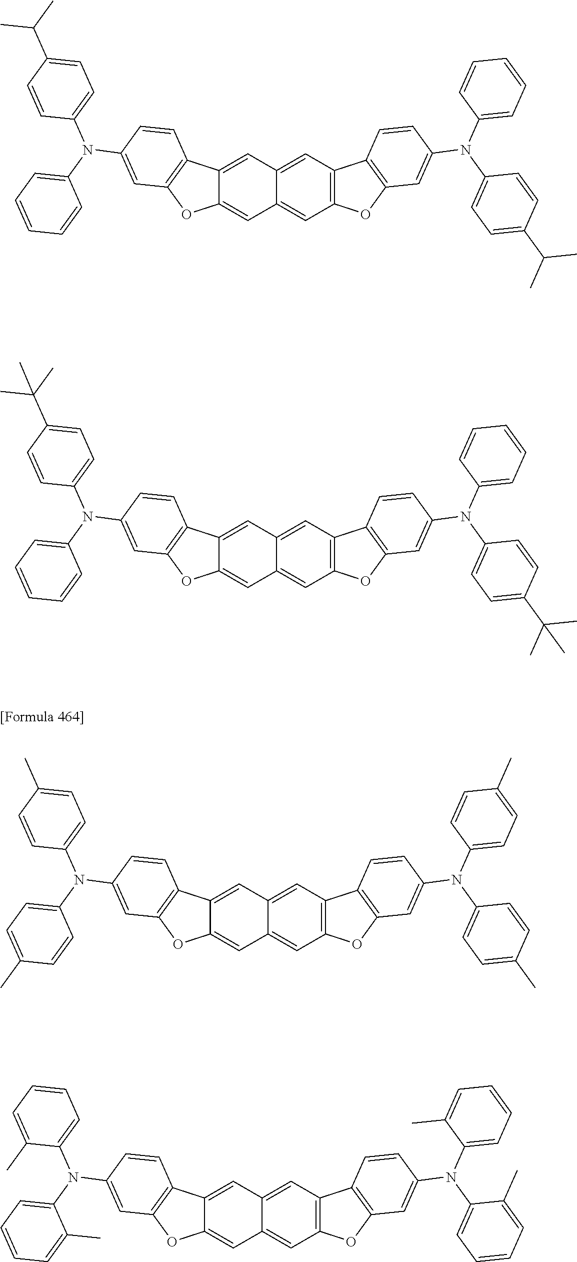

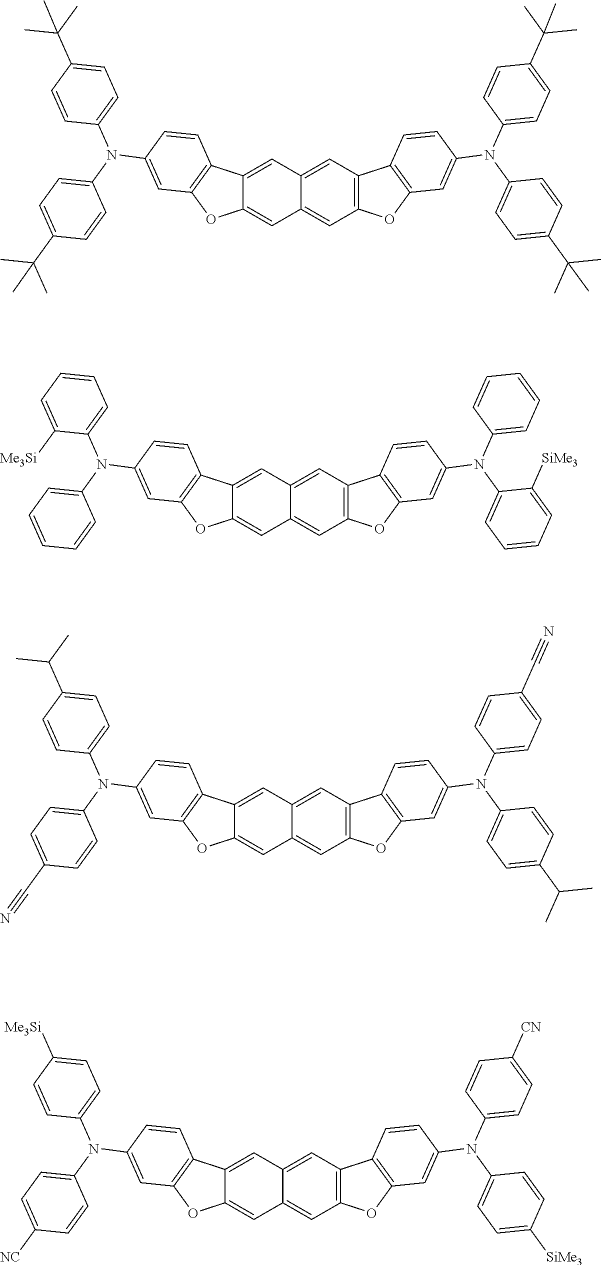 [SMILES: CC(C)(C)c1ccc(N(c2ccc(C(C)(C)C)cc2)c2ccc3c(c2)oc2cc4cc5oc6cc(N(c7ccc(C(C)(C)C)cc7)c7ccc(C(C)(C)C)cc7)ccc6c5cc4cc23)cc1 ; C[Si](C)(C)c1ccccc1N(c1ccccc1)c1ccc2c(c1)oc1cc3cc4oc5cc(N(c6ccccc6)c6ccccc6[Si](C)(C)C)ccc5c4cc3cc12 ; CC(C)c1ccc(N(c2ccc(C#N)cc2)c2ccc3c(c2)oc2cc4cc5oc6cc(N(c7ccc(C#N)cc7)c7ccc(C(C)C)cc7)ccc6c5cc4cc23)cc1 ; C[Si](C)(C)c1ccc(N(c2ccc(C#N)cc2)c2ccc3c(c2)oc2cc4cc5oc6cc(N(c7ccc(C#N)cc7)c7ccc([Si](C)(C)C)cc7)ccc6c5cc4cc23)cc1]

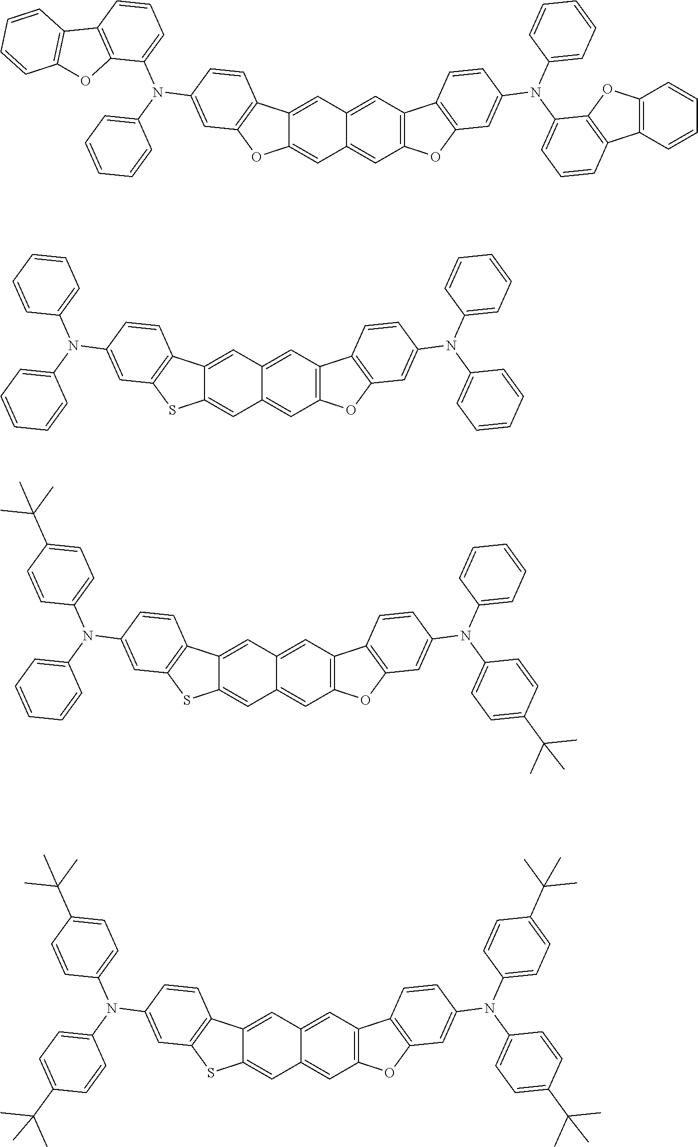

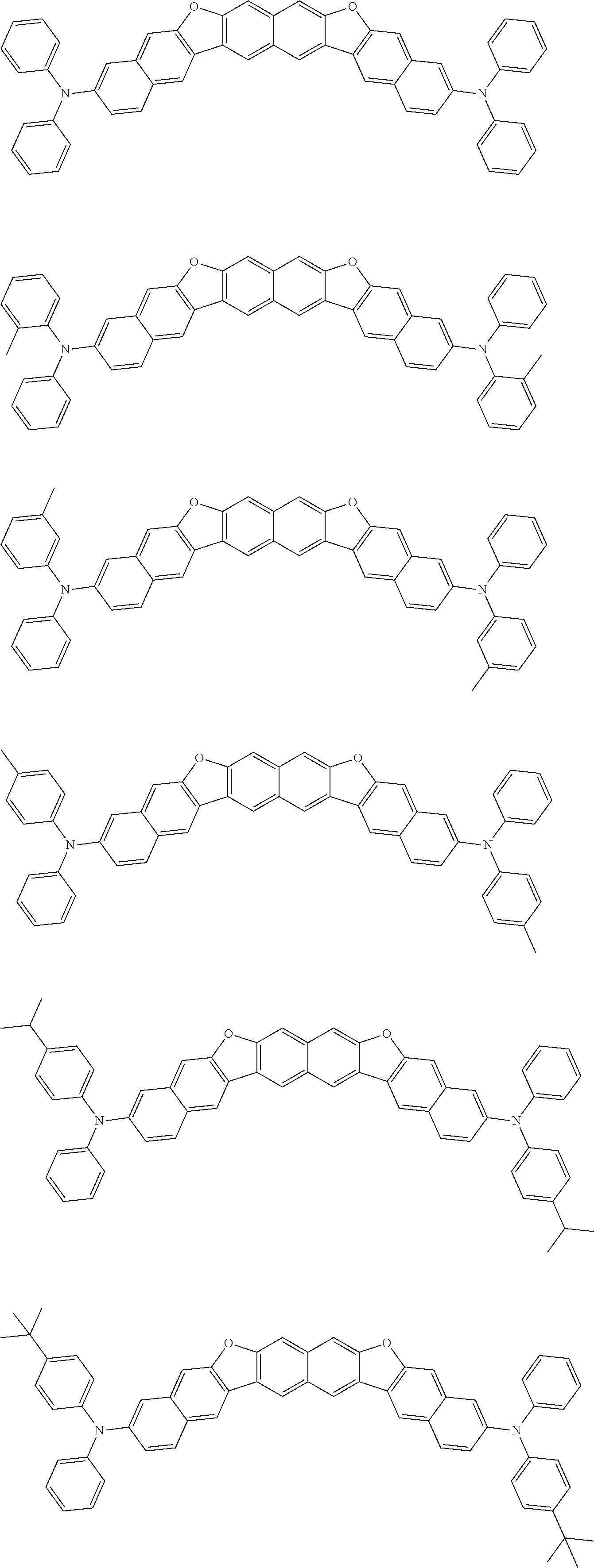

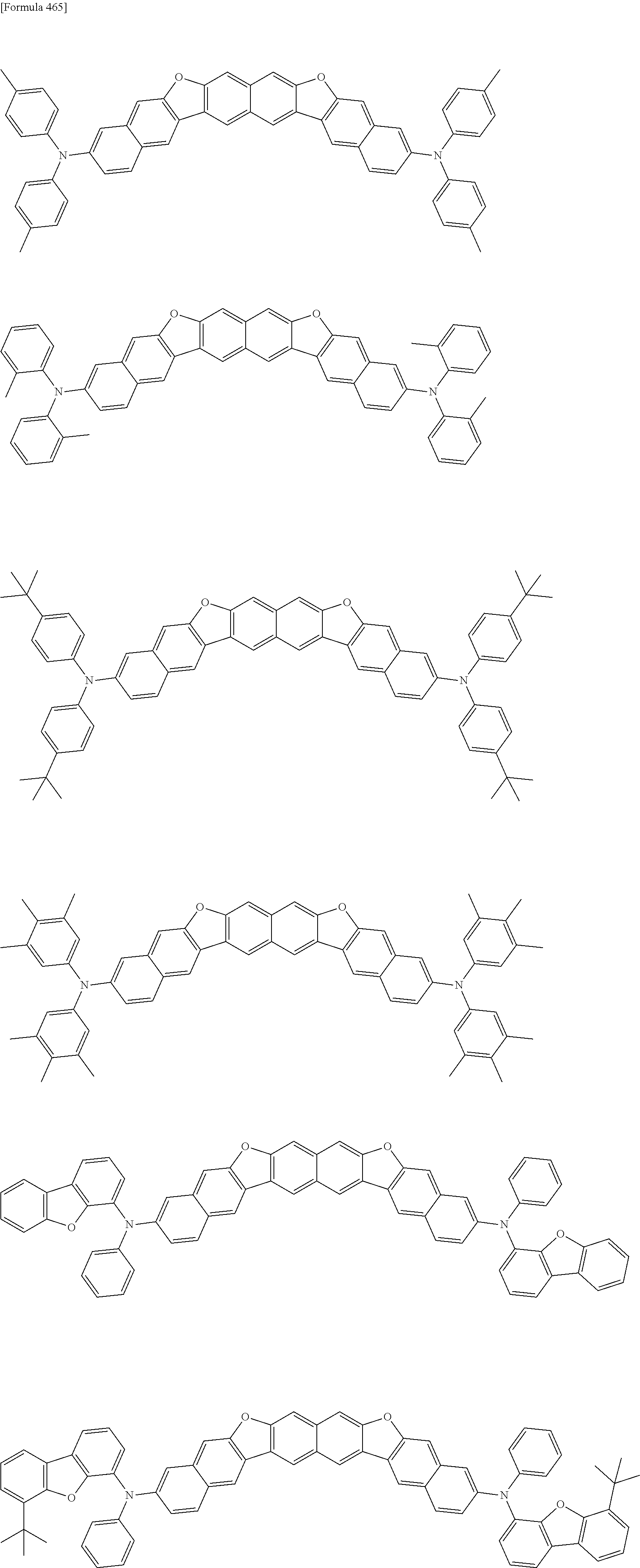

Compound Represented by Formula (8)

A compound represented by the formula (8) will be described below.

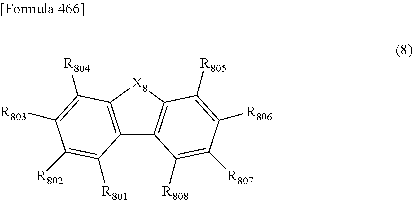

In the formula (8): at least one combination of R₈₀₁ and R₈₀₂, R₈₀₂ and R₈₀₃, or R₈₀₃ and R₈₀₄ are mutually bonded to form a divalent group represented by a formula (82) below; and
at least one combination of R₈₀₅ and R₈₀₆, R₈₀₆ and R₈₀₇, or R₈₀₇ and R₈₀₈ are mutually bonded to form a divalent group represented by a formula (83) below.

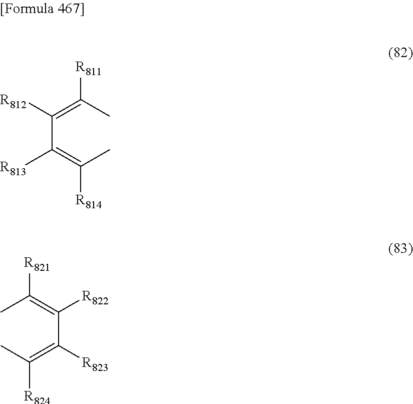

At least one of R₈₀₁ to R₈₀₄ not forming the divalent group represented by the formula (82) or R₈₁₁ to R₈₁₄ is a monovalent group represented by a formula (84) below;
at least one of R₈₀₅ to R₈₀₅ not forming the divalent group represented by the formula (83) or R₈₂₁ to R₈₂₄ is a monovalent group represented by a formula (84) below;
X₈ is an oxygen atom, a sulfur atom, or NR₈₀₉; and
R₈₀₁ to R₈₀₅ not forming the divalent group represented by the formula (82) or (83) and not being the monovalent group represented by the formula (84), R₈₁₁ to R₈₁₄ and R₈₂₁ to R₈₂₄ not being the monovalent group represented by the formula (84), and R₈₀₉ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group Represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

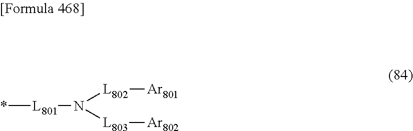

In the formula (84): Ar₈₀₁ and Ar₈₀₂ are each independently a substituted or unsubstituted aryl group having 6 to 50 ring carbon atoms, or a substituted or unsubstituted heterocyclic group having 5 to 50 ring atoms;
L₈₀₁ to L₈₀₃ are each independently a single bond, a substituted or unsubstituted arylene group having 6 to 30 ring carbon atoms, a substituted or unsubstituted divalent heterocyclic group having 5 to 30 ring atoms, or a divalent linking group formed by bonding two, three or four groups selected from the group consisting of a substituted or unsubstituted arylene group having 6 to 30 ring carbon atoms and a substituted or unsubstituted divalent heterocyclic group having 5 to 30 ring atoms; and
* in the formula (84) represents a bonding position to the cyclic structure represented by the formula (8), a group represented by the formula (82), or a group represented by the formula (83).

In the formula (8), the positions for the divalent group represented by the formula (82) and the divalent group represented by the formula (83) to be formed are not specifically limited but the divalent groups may be formed at any possible positions on R₈₀₁ to R₈₀₈.

In an exemplary embodiment, a compound represented by the formula (8) is represented by any one of formulae (81-1) to (81-6) below.

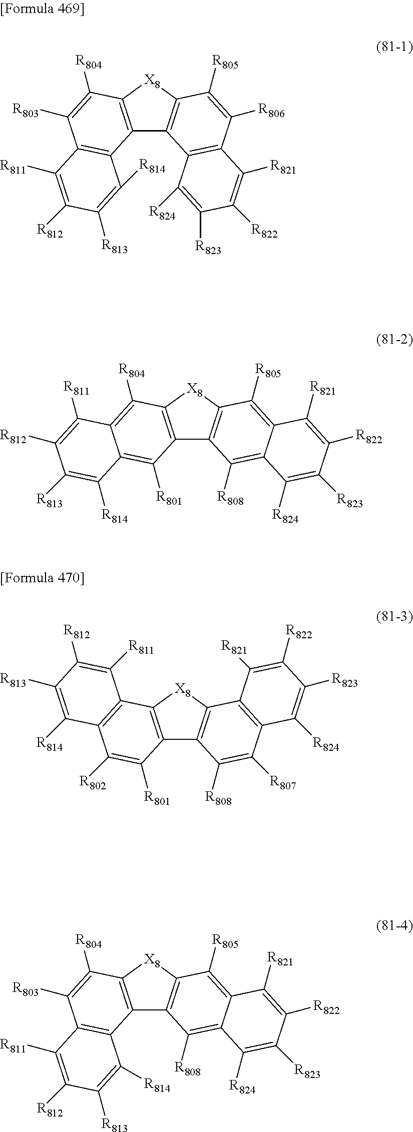

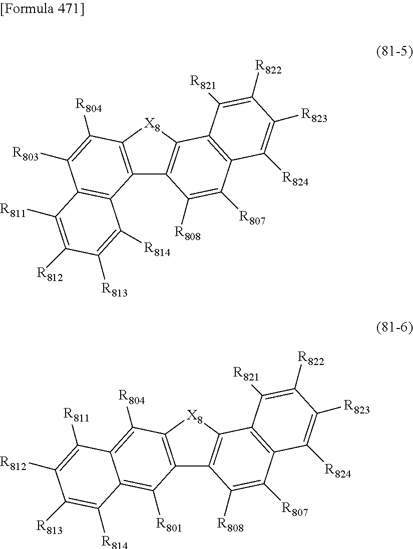

In the formulae (81-1) to (81-6):
X₈ represents the same as X₈ in the formula (8);
at least two of R₈₀₁ to R₈₂₄ are each a monovalent group represented by the formula (84); and
R₈₀₁ to R₈₂₄ that are not the monovalent group represented by the formula (84) are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group Represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In an exemplary embodiment, a compound represented by the formula (8) is represented by any one of formulae (81-7) to (81-18) below.

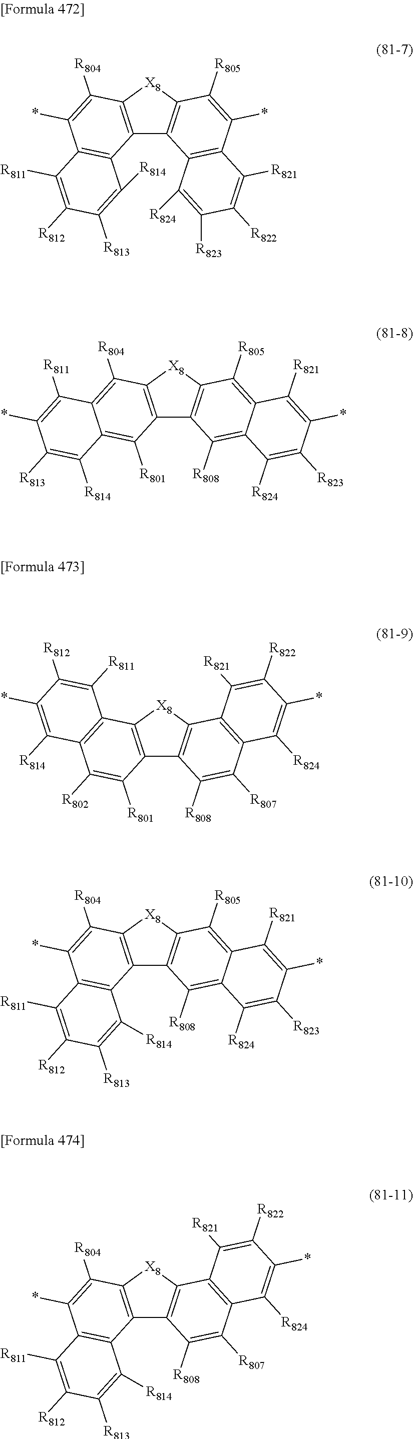

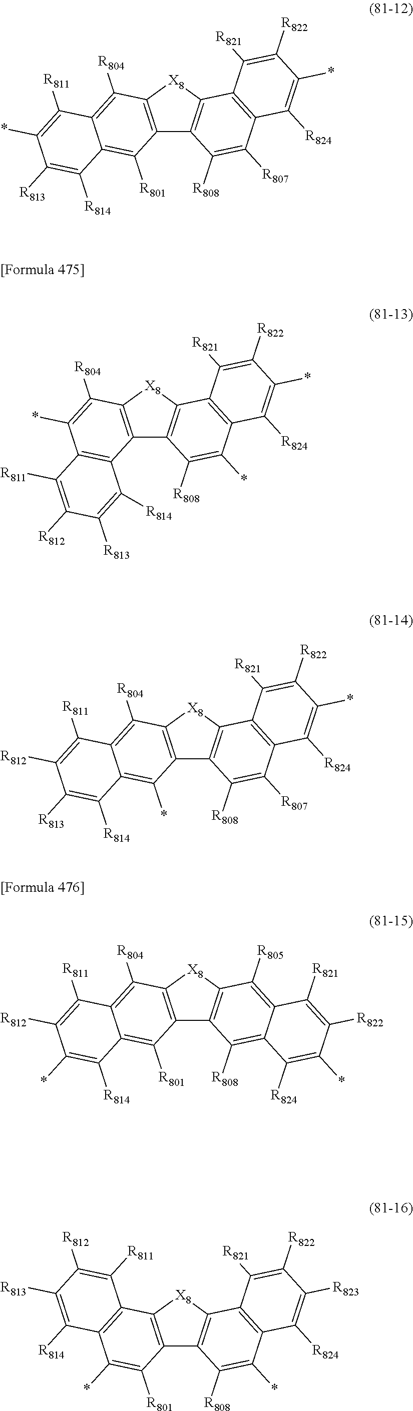

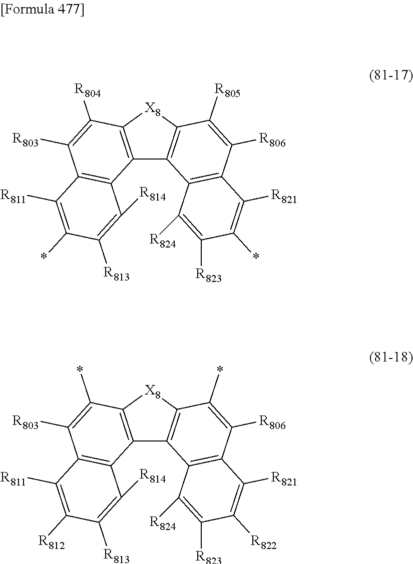

In the formulae (81-7) to (81-18):
X₈ represents the same as X₈ in the formula (8);
* is a single bond bonded to a monovalent group represented by the formula (84); and
R₈₀₁ to R₈₂₄ each independently represent the same as R₈₀₁ to R₈₂₄ that are each not a monovalent group represented by the formula (84) in the formulae (81-1) to (81-6).
R₈₀₁ to R₈₀₅ not forming the divalent group represented by the formula (82) or (83) and not being the monovalent group represented by the formula (84), and R₈₁₁ to R₈₁₄ and R₈₂₁ to R₈₂₄ not being the monovalent group represented by the formula (84) are preferably each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

The monovalent group represented by the formula (84) is preferably represented by a formula (85) or (86) below.

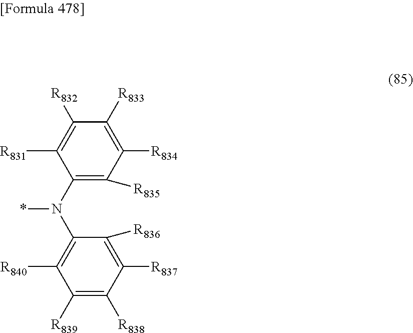

In the formula (85): R₈₃₁ to R₈₄₀ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group Represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms; and
* in the formula (85) represents the same as * in the formula (84).

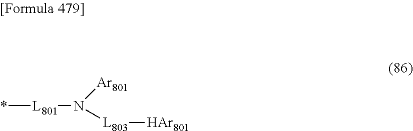

In the formula (86): Ar₈₀₁, L₈₀₁, and L₈₀₃ represent the same as Ar₈₀₁, L₈₀₁, and L₈₀₃ in the formula (84); and HAr₈₀₁ is a structure represented by a formula (87).

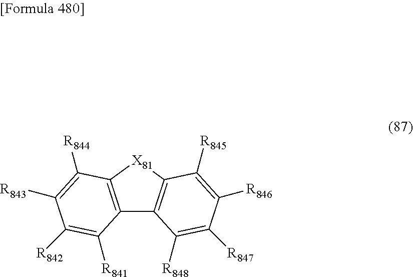

In the formula (87):
X₈₁ is an oxygen atom or a sulfur atom;
one of R₈₄₁ to R₈₄₈ is a single bond with L₈₀₃; and
R₈₄₁ to R₈₄₈ not being the single bond are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group Represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

Specific examples of a compound represented by the formula (8) include compounds shown below as well as the compounds disclosed in WO 2014/104144.

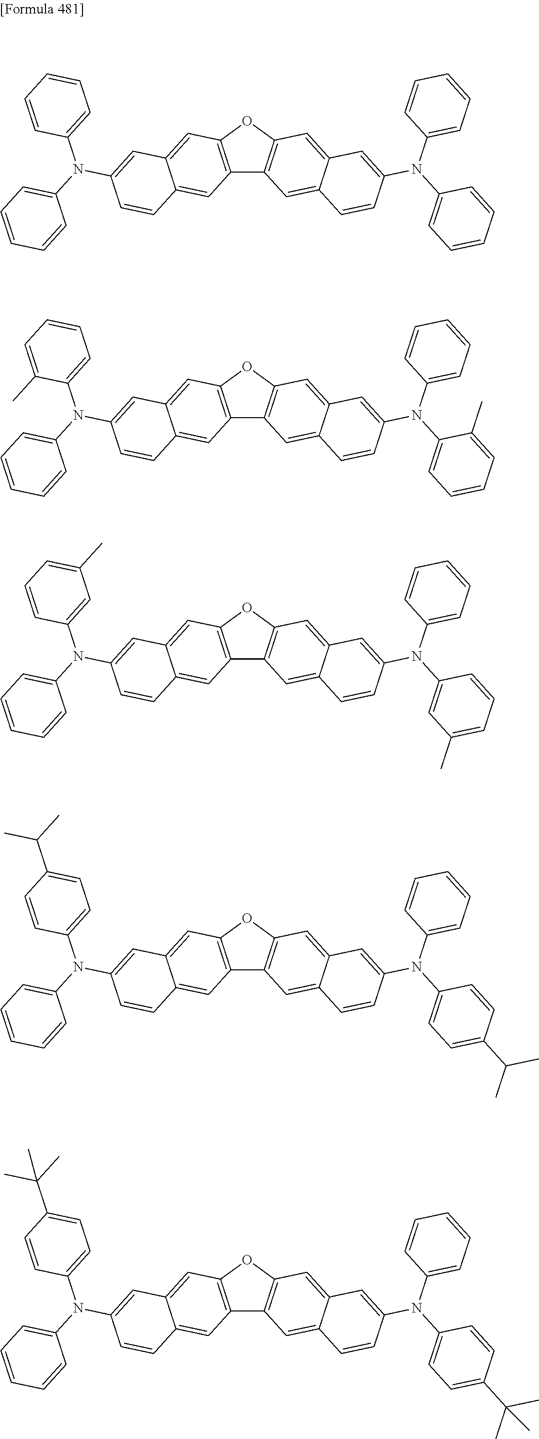 [SMILES: c1ccc(N(c2ccccc2)c2ccc3cc4c(cc3c2)oc2cc3cc(N(c5ccccc5)c5ccccc5)ccc3cc24)cc1 ; Cc1ccccc1N(c1ccccc1)c1ccc2cc3c(cc2c1)oc1cc2cc(N(c4ccccc4)c4ccccc4C)ccc2cc13 ; Cc1cccc(N(c2ccccc2)c2ccc3cc4c(cc3c2)oc2cc3cc(N(c5ccccc5)c5cccc(C)c5)ccc3cc24)c1 ; CC(C)c1ccc(N(c2ccccc2)c2ccc3cc4c(cc3c2)oc2cc3cc(N(c5ccccc5)c5ccc(C(C)C)cc5)ccc3cc24)cc1 ; CC(C)(C)c1ccc(N(c2ccccc2)c2ccc3cc4c(cc3c2)oc2cc3cc(N(c5ccccc5)c5ccc(C(C)(C)C)cc5)ccc3cc24)cc1]

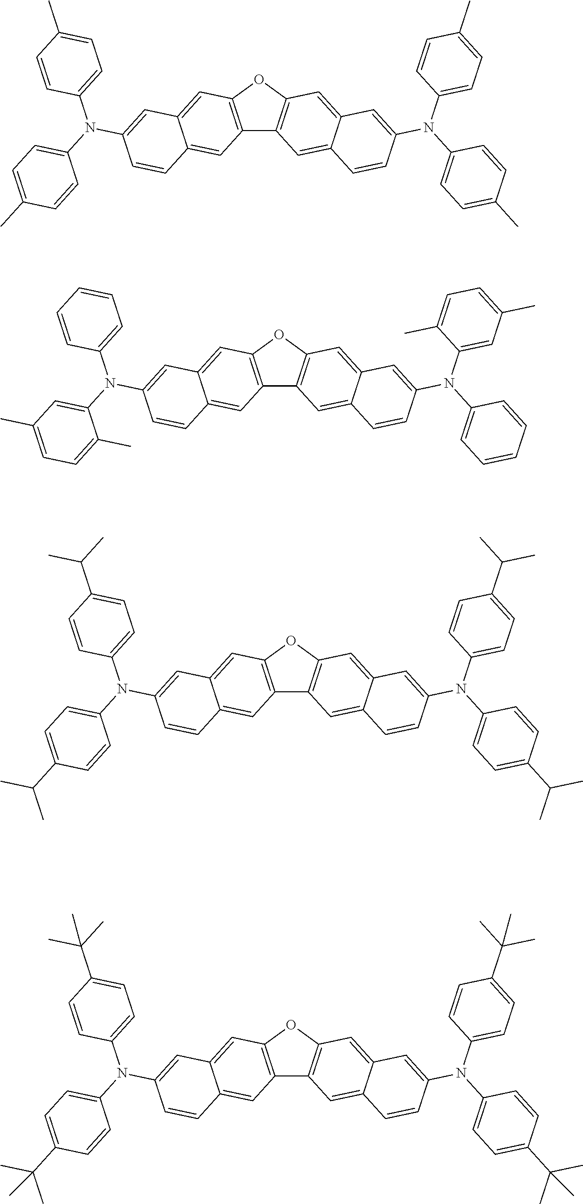

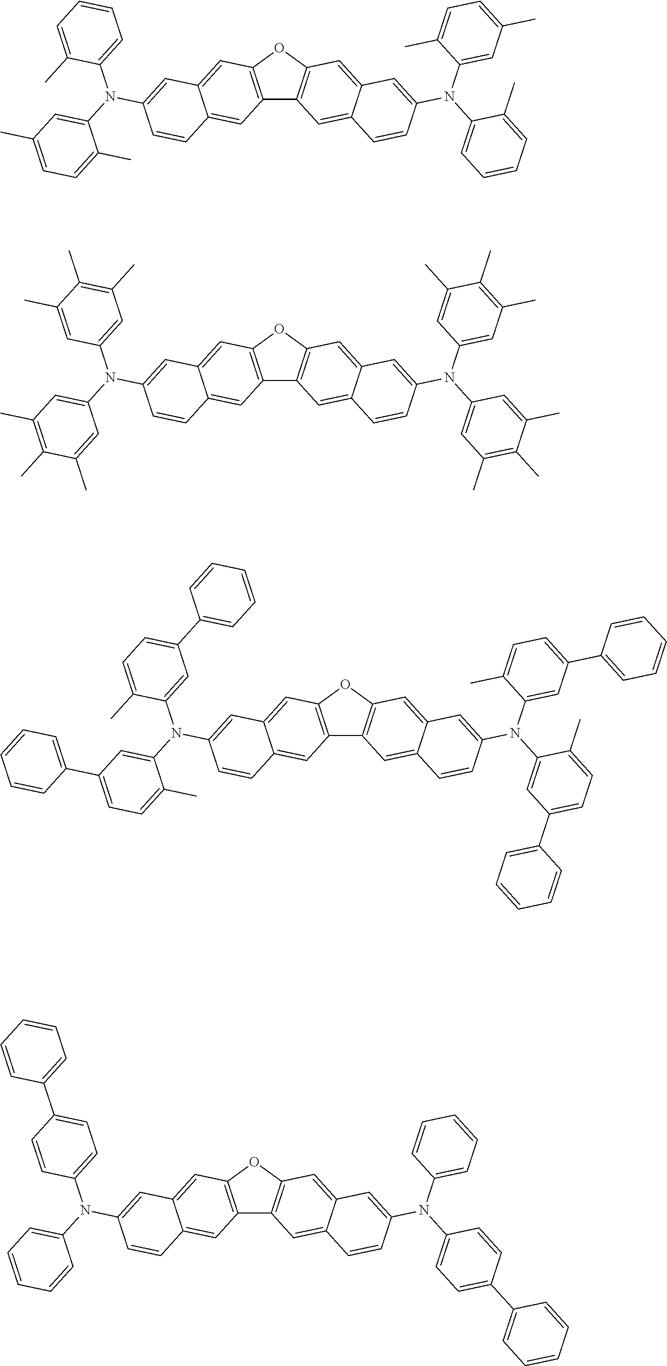

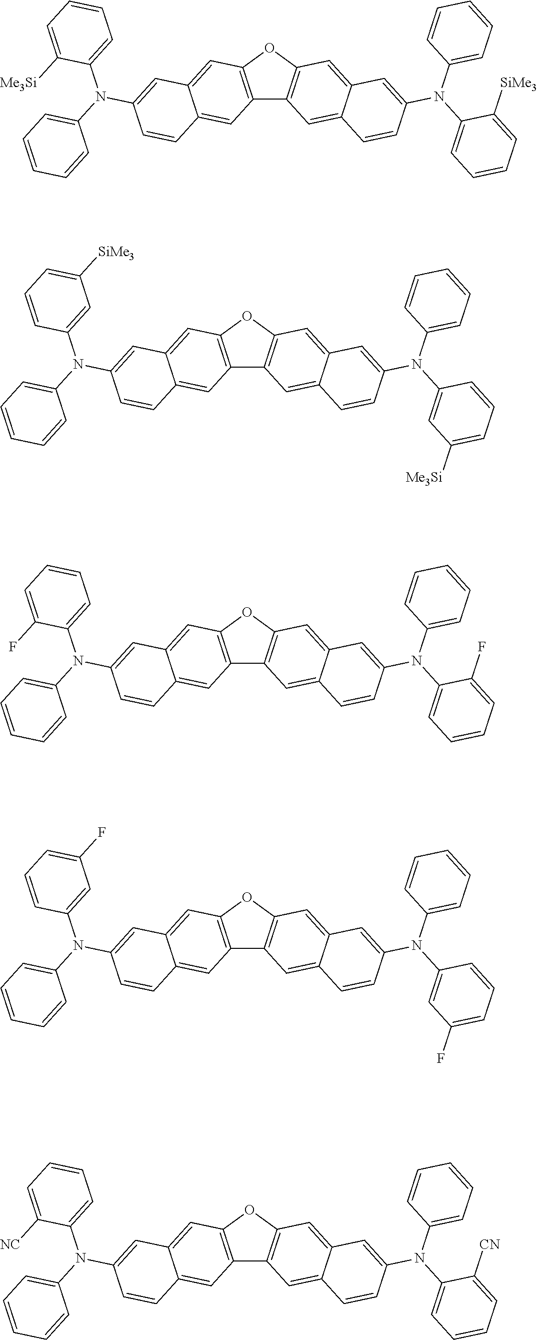 [SMILES: C[Si](C)(C)c1ccccc1N(c1ccccc1)c1ccc2cc3c(cc2c1)oc1cc2cc(N(c4ccccc4)c4ccccc4[Si](C)(C)C)ccc2cc13 ; C[Si](C)(C)c1cccc(N(c2ccccc2)c2ccc3cc4c(cc3c2)oc2cc3cc(N(c5ccccc5)c5cccc([Si](C)(C)C)c5)ccc3cc24)c1 ; Fc1ccccc1N(c1ccccc1)c1ccc2cc3c(cc2c1)oc1cc2cc(N(c4ccccc4)c4ccccc4F)ccc2cc13 ; Fc1cccc(N(c2ccccc2)c2ccc3cc4c(cc3c2)oc2cc3cc(N(c5ccccc5)c5cccc(F)c5)ccc3cc24)c1 ; N#Cc1ccccc1N(c1ccccc1)c1ccc2cc3c(cc2c1)oc1cc2cc(N(c4ccccc4)c4ccccc4C#N)ccc2cc13]

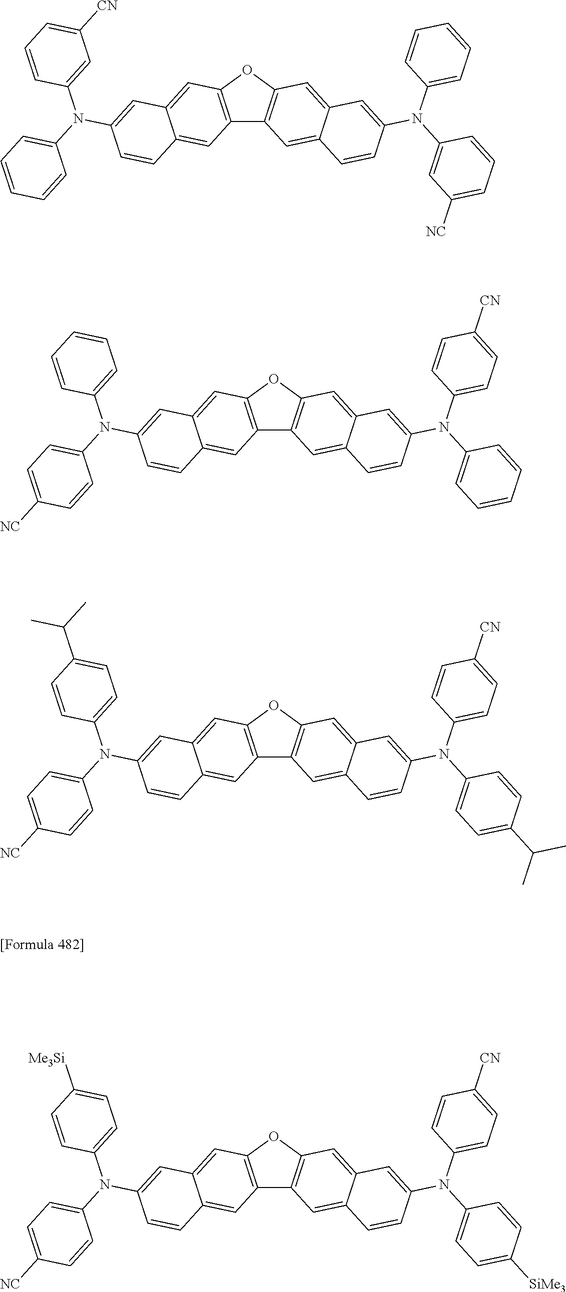 [SMILES: N#Cc1cccc(N(c2ccccc2)c2ccc3cc4c(cc3c2)oc2cc3cc(N(c5ccccc5)c5cccc(C#N)c5)ccc3cc24)c1 ; N#Cc1ccc(N(c2ccccc2)c2ccc3cc4c(cc3c2)oc2cc3cc(N(c5ccccc5)c5ccc(C#N)cc5)ccc3cc24)cc1 ; CC(C)c1ccc(N(c2ccc(C#N)cc2)c2ccc3cc4c(cc3c2)oc2cc3cc(N(c5ccc(C#N)cc5)c5ccc(C(C)C)cc5)ccc3cc24)cc1 ; C[Si](C)(C)c1ccc(N(c2ccc(C#N)cc2)c2ccc3cc4c(cc3c2)oc2cc3cc(N(c5ccc(C#N)cc5)c5ccc([Si](C)(C)C)cc5)ccc3cc24)cc1]

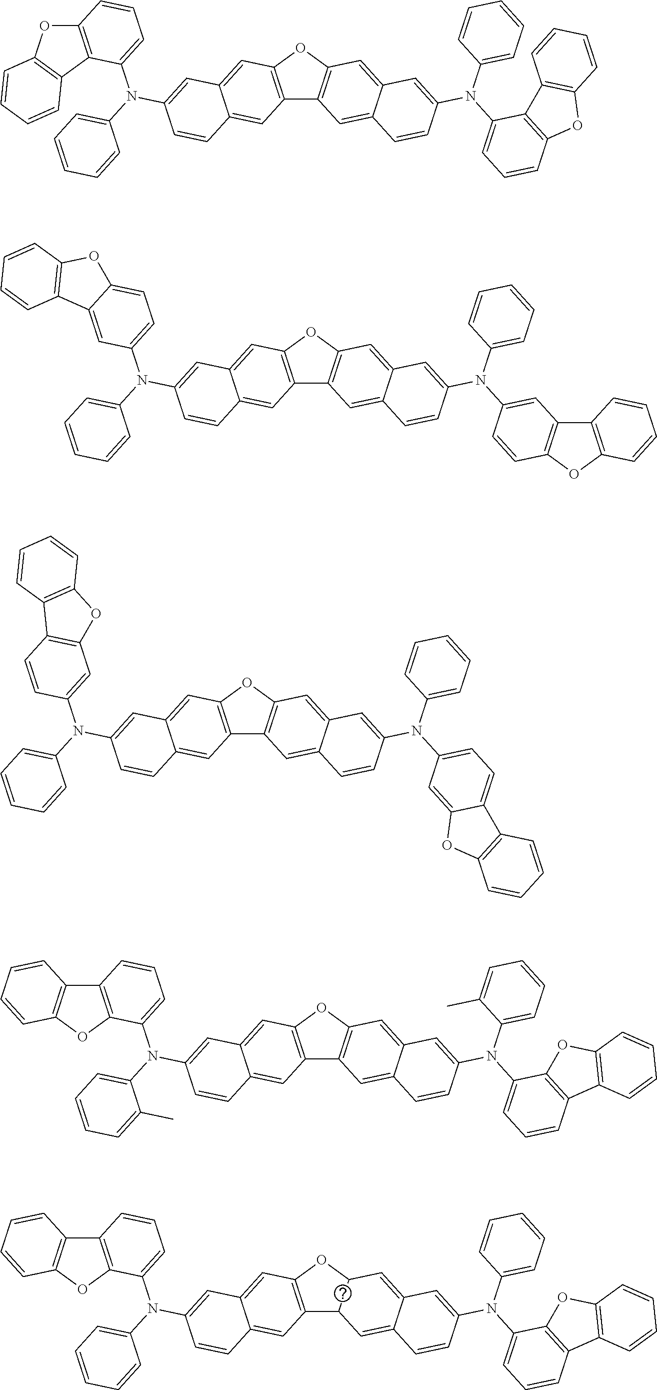

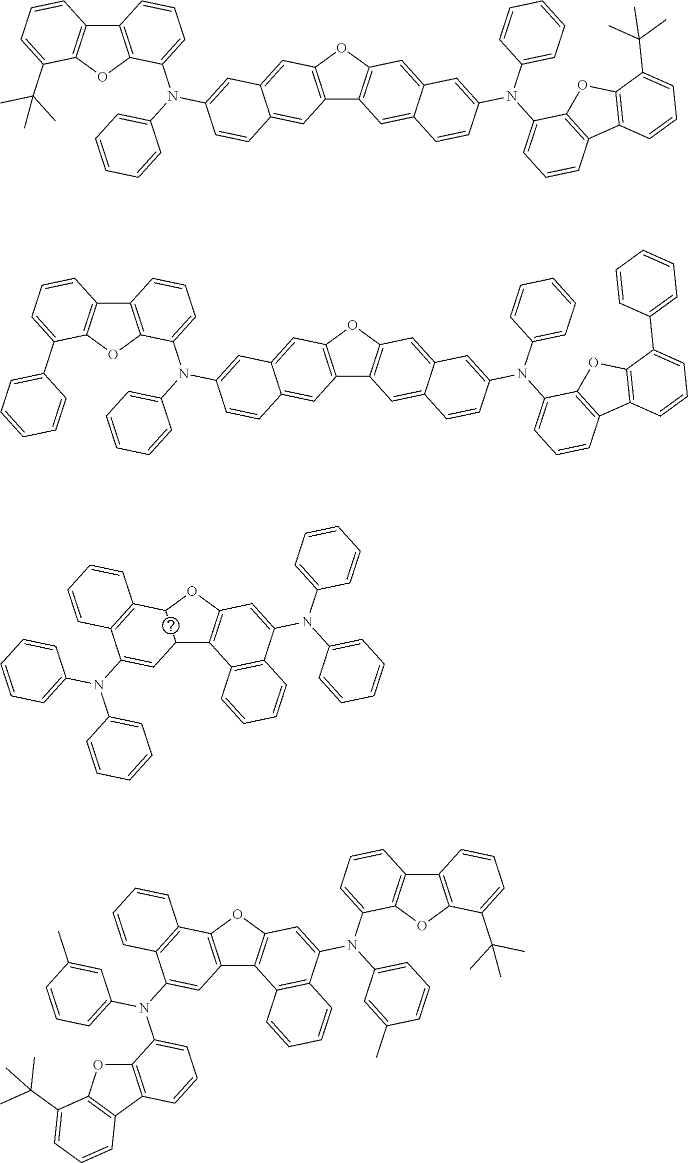 [SMILES: CC(C)(C)c1cccc2c1oc1c(N(c3ccccc3)c3ccc4cc5c(cc4c3)oc3cc4cc(N(c6ccccc6)c6cccc7c6oc6c(C(C)(C)C)cccc67)ccc4cc35)cccc12 ; c1ccc(-c2cccc3c2oc2c(N(c4ccccc4)c4ccc5cc6c(cc5c4)oc4cc5cc(N(c7ccccc7)c7cccc8c7oc7c(-c9ccccc9)cccc78)ccc5cc46)cccc23)cc1 ; C1=C(N(c2ccccc2)c2ccccc2)c2ccccc2C2Oc3cc(N(c4ccccc4)c4ccccc4)c4ccccc4c3C12 ; Cc1cccc(N(c2cc3c(oc4cc(N(c5cccc(C)c5)c5cccc6c5oc5c(C(C)(C)C)cccc56)c5ccccc5c43)c3ccccc23)c2cccc3c2oc2c(C(C)(C)C)cccc23)c1]

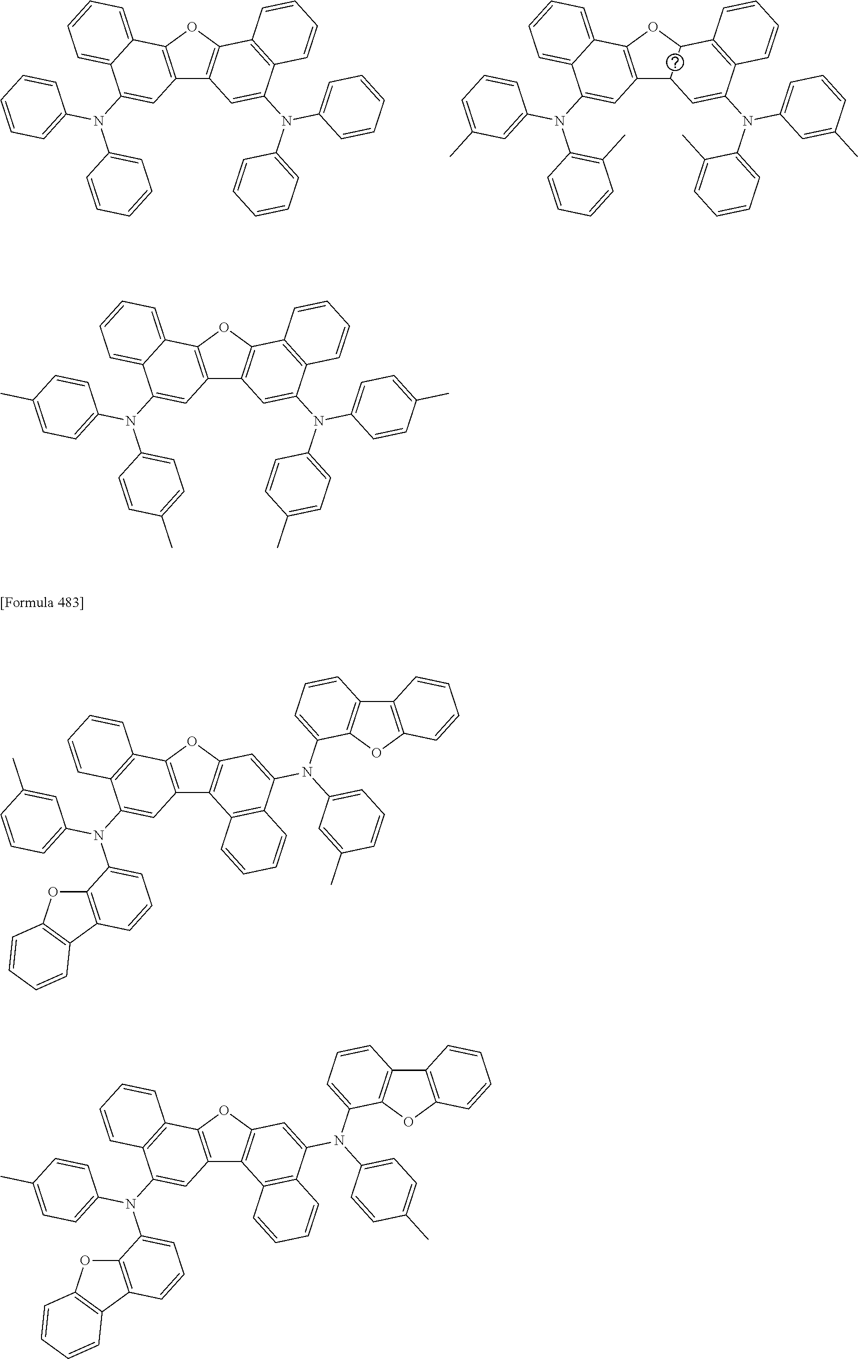

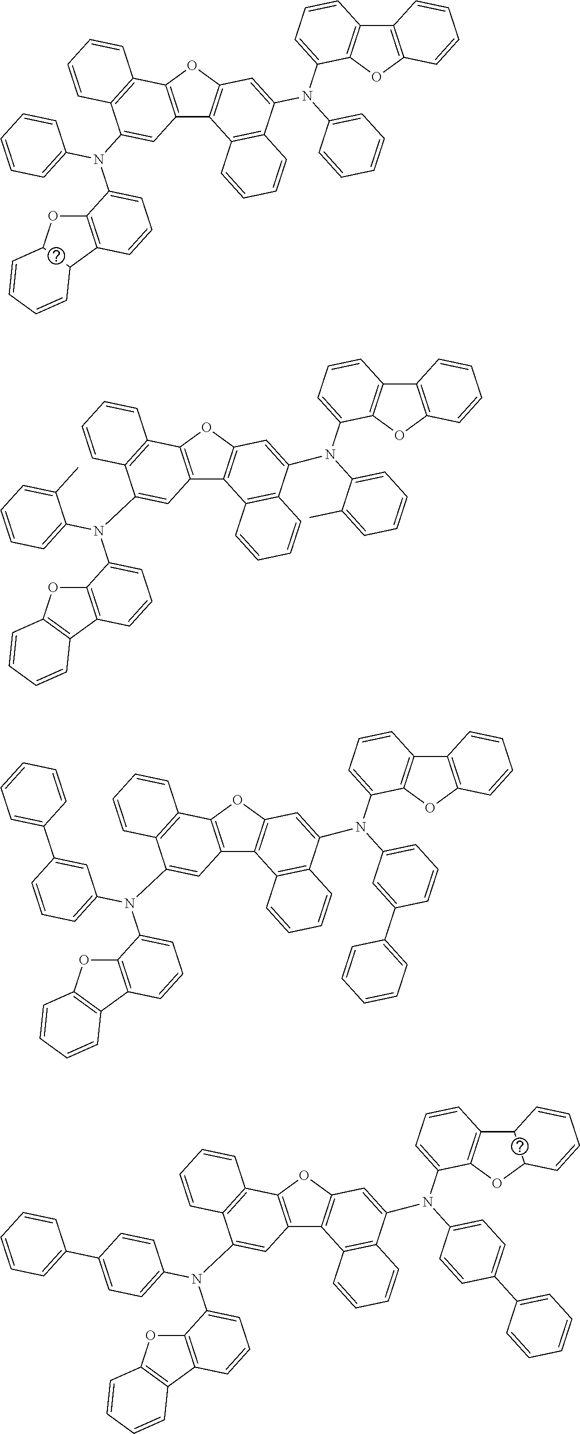 [SMILES: C1=CC2Oc3c(cccc3N(c3ccccc3)c3cc4c(oc5cc(N(c6ccccc6)c6cccc7c6oc6ccccc67)c6ccccc6c54)c4ccccc34)C2C=C1 ; Cc1ccccc1N(c1cc2c(oc3cc(N(c4ccccc4C)c4cccc5c4oc4ccccc45)c4ccccc4c32)c2ccccc12)c1cccc2c1oc1ccccc12 ; c1ccc(-c2cccc(N(c3cc4c(oc5cc(N(c6cccc(-c7ccccc7)c6)c6cccc7c6oc6ccccc67)c6ccccc6c54)c4ccccc34)c3cccc4c3oc3ccccc34)c2)cc1 ; C1=CC2Oc3c(cccc3N(c3ccc(-c4ccccc4)cc3)c3cc4oc5c6ccccc6c(N(c6ccc(-c7ccccc7)cc6)c6cccc7c6oc6ccccc67)cc5c4c4ccccc34)C2C=C1]

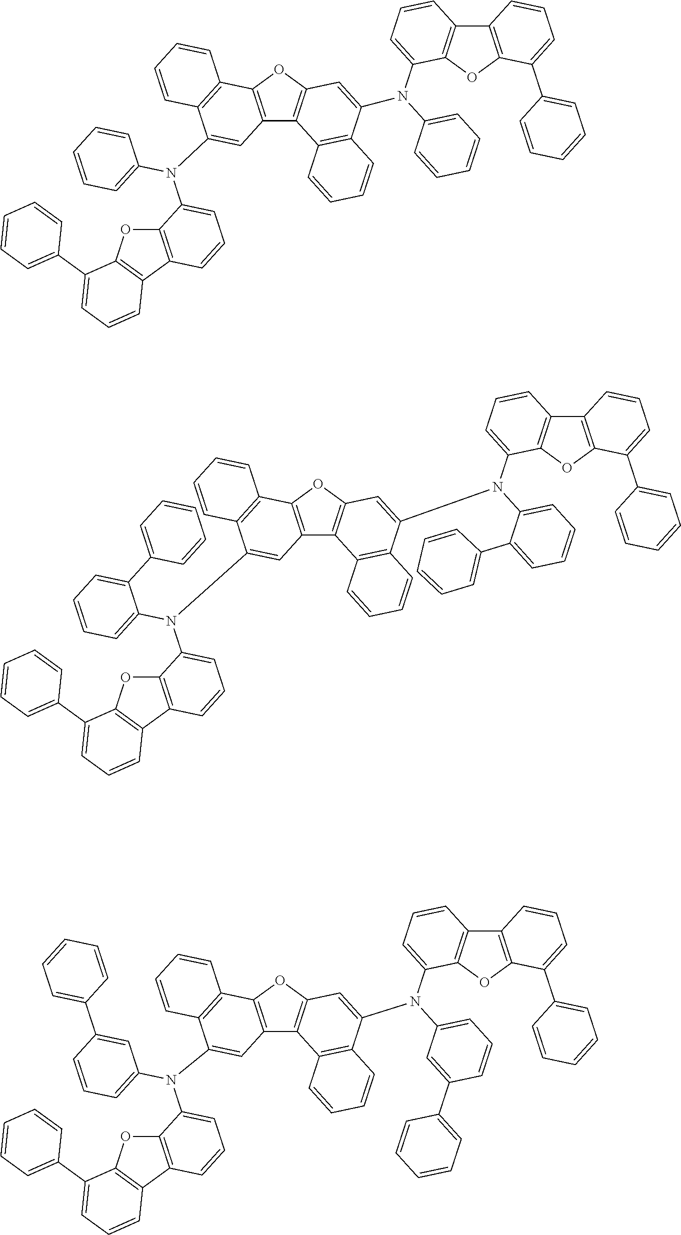 [SMILES: c1ccc(-c2cccc3c2oc2c(N(c4ccccc4)c4cc5c(oc6cc(N(c7ccccc7)c7cccc8c7oc7c(-c9ccccc9)cccc78)c7ccccc7c65)c5ccccc45)cccc23)cc1 ; c1ccc(-c2ccccc2N(c2cc3c(oc4cc(N(c5ccccc5-c5ccccc5)c5cccc6c5oc5c(-c7ccccc7)cccc56)c5ccccc5c43)c3ccccc23)c2cccc3c2oc2c(-c4ccccc4)cccc23)cc1 ; c1ccc(-c2cccc(N(c3cc4c(oc5cc(N(c6cccc(-c7ccccc7)c6)c6cccc7c6oc6c(-c8ccccc8)cccc67)c6ccccc6c54)c4ccccc34)c3cccc4c3oc3c(-c5ccccc5)cccc34)c2)cc1]

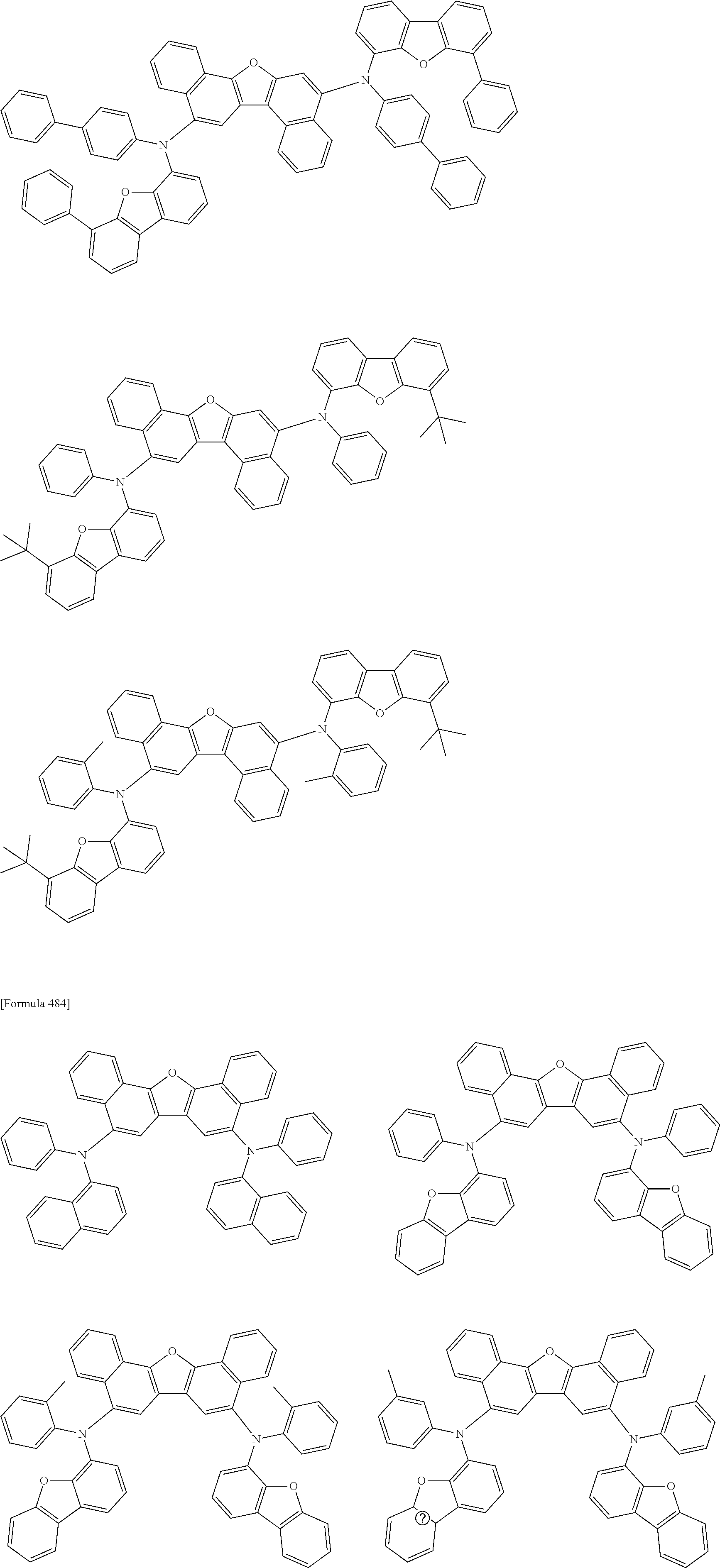

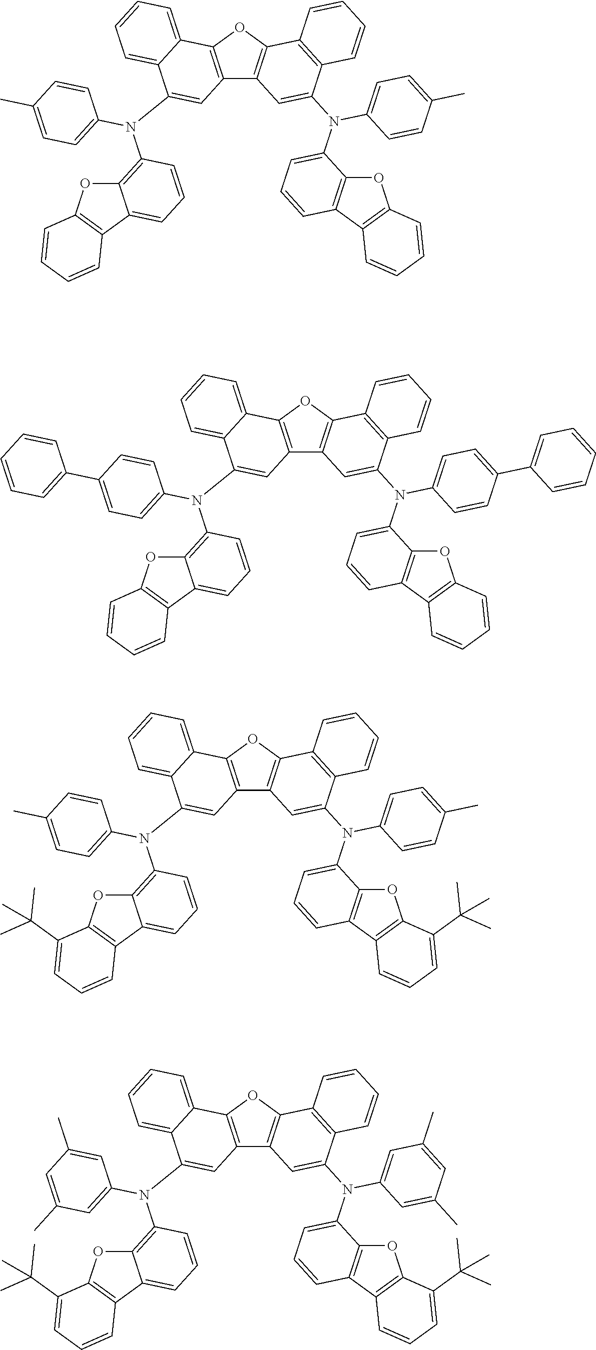

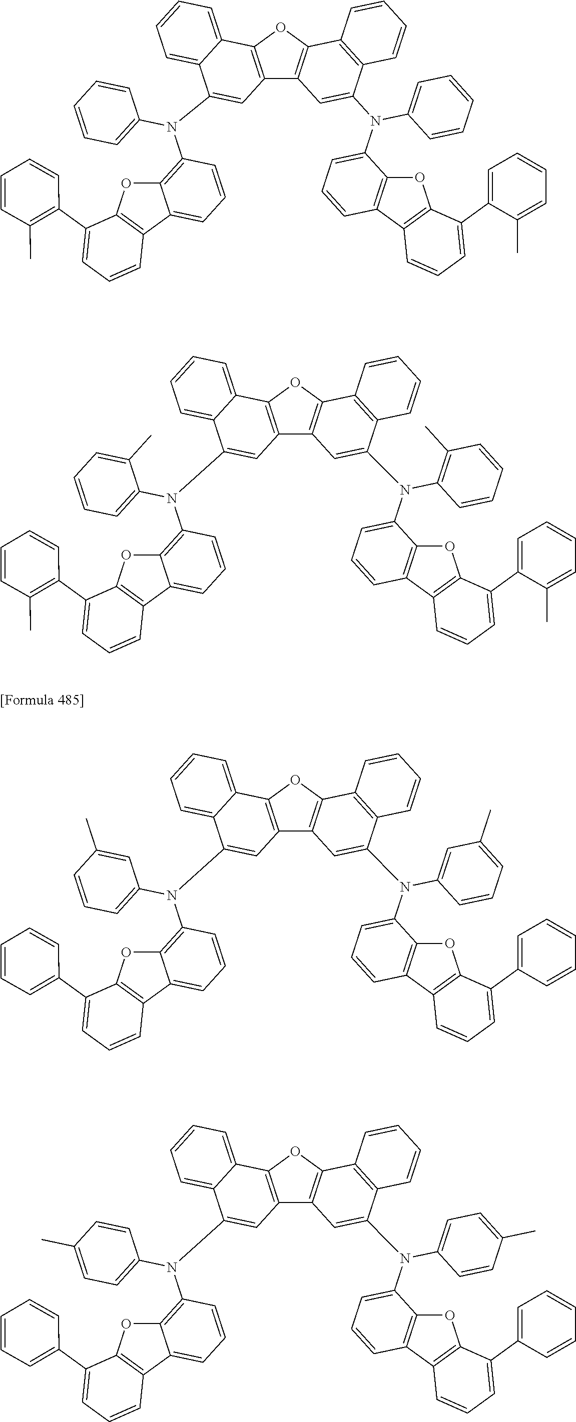 [SMILES: Cc1ccccc1-c1cccc2c1oc1c(N(c3ccccc3)c3cc4c5cc(N(c6ccccc6)c6cccc7c6oc6c(-c8ccccc8C)cccc67)c6ccccc6c5oc4c4ccccc34)cccc12 ; Cc1ccccc1-c1cccc2c1oc1c(N(c3ccccc3C)c3cc4c5cc(N(c6ccccc6C)c6cccc7c6oc6c(-c8ccccc8C)cccc67)c6ccccc6c5oc4c4ccccc34)cccc12 ; Cc1cccc(N(c2cc3c4cc(N(c5cccc(C)c5)c5cccc6c5oc5c(-c7ccccc7)cccc56)c5ccccc5c4oc3c3ccccc23)c2cccc3c2oc2c(-c4ccccc4)cccc23)c1 ; Cc1ccc(N(c2cc3c4cc(N(c5ccc(C)cc5)c5cccc6c5oc5c(-c7ccccc7)cccc56)c5ccccc5c4oc3c3ccccc23)c2cccc3c2oc2c(-c4ccccc4)cccc23)cc1]

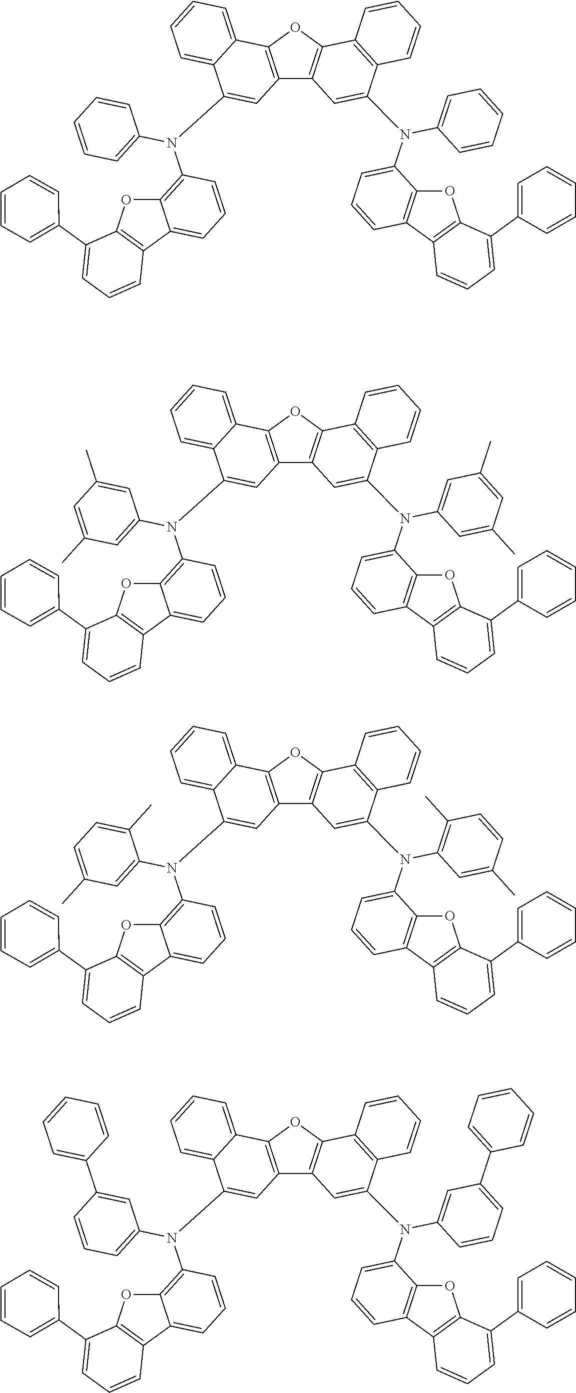

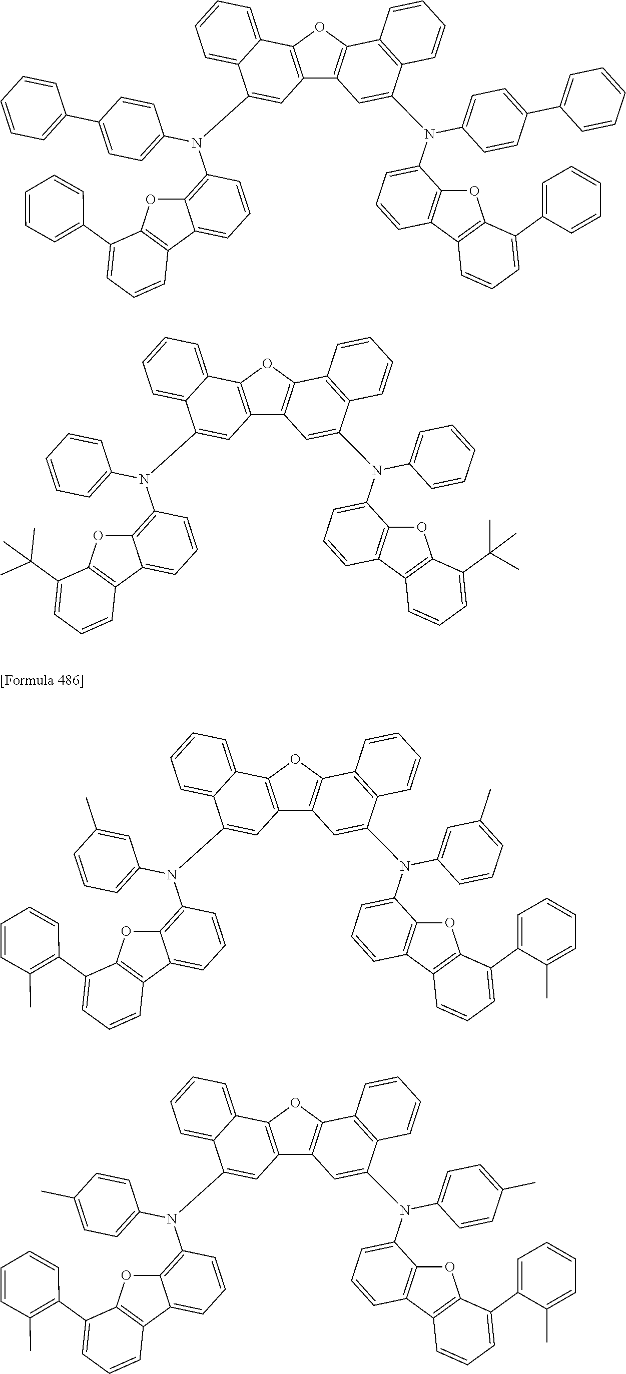

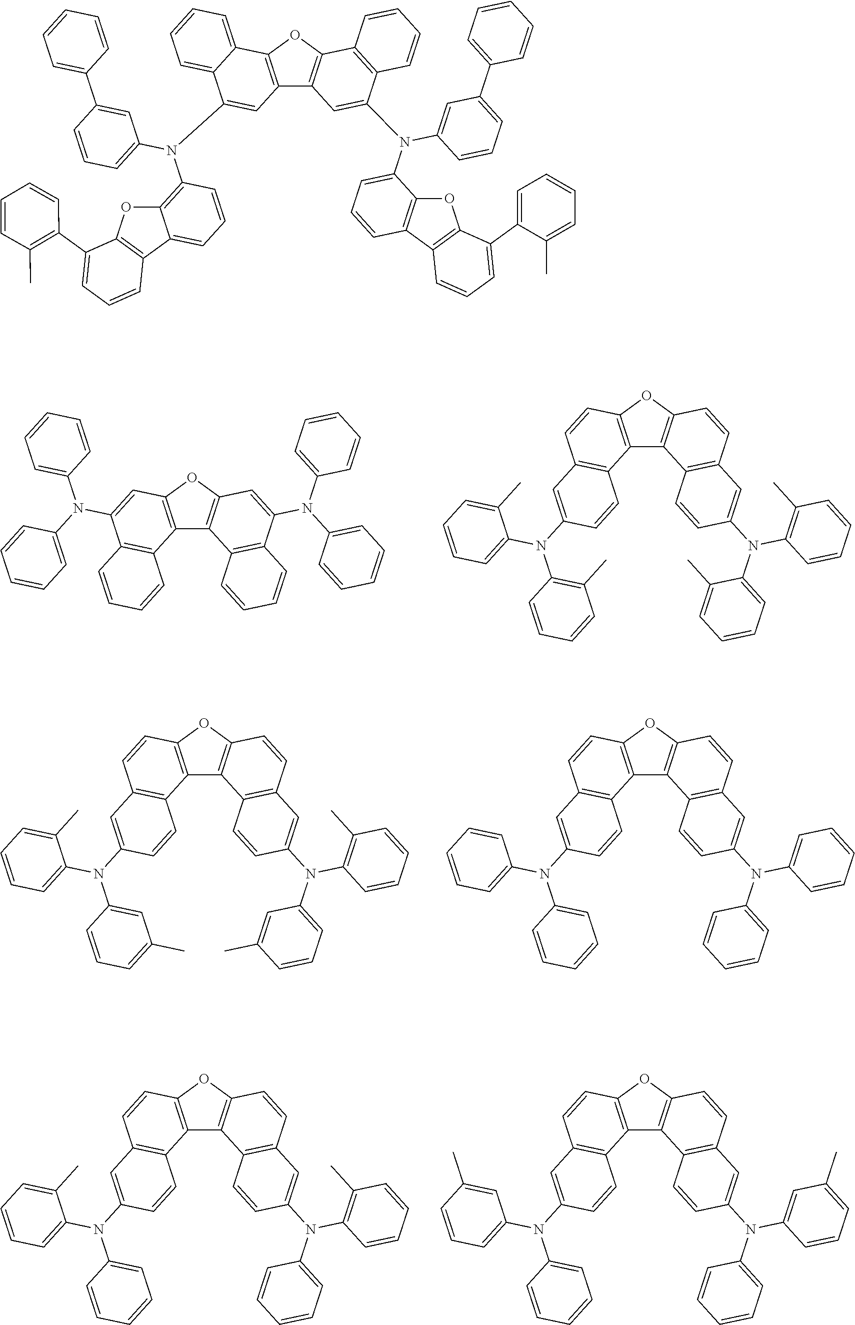

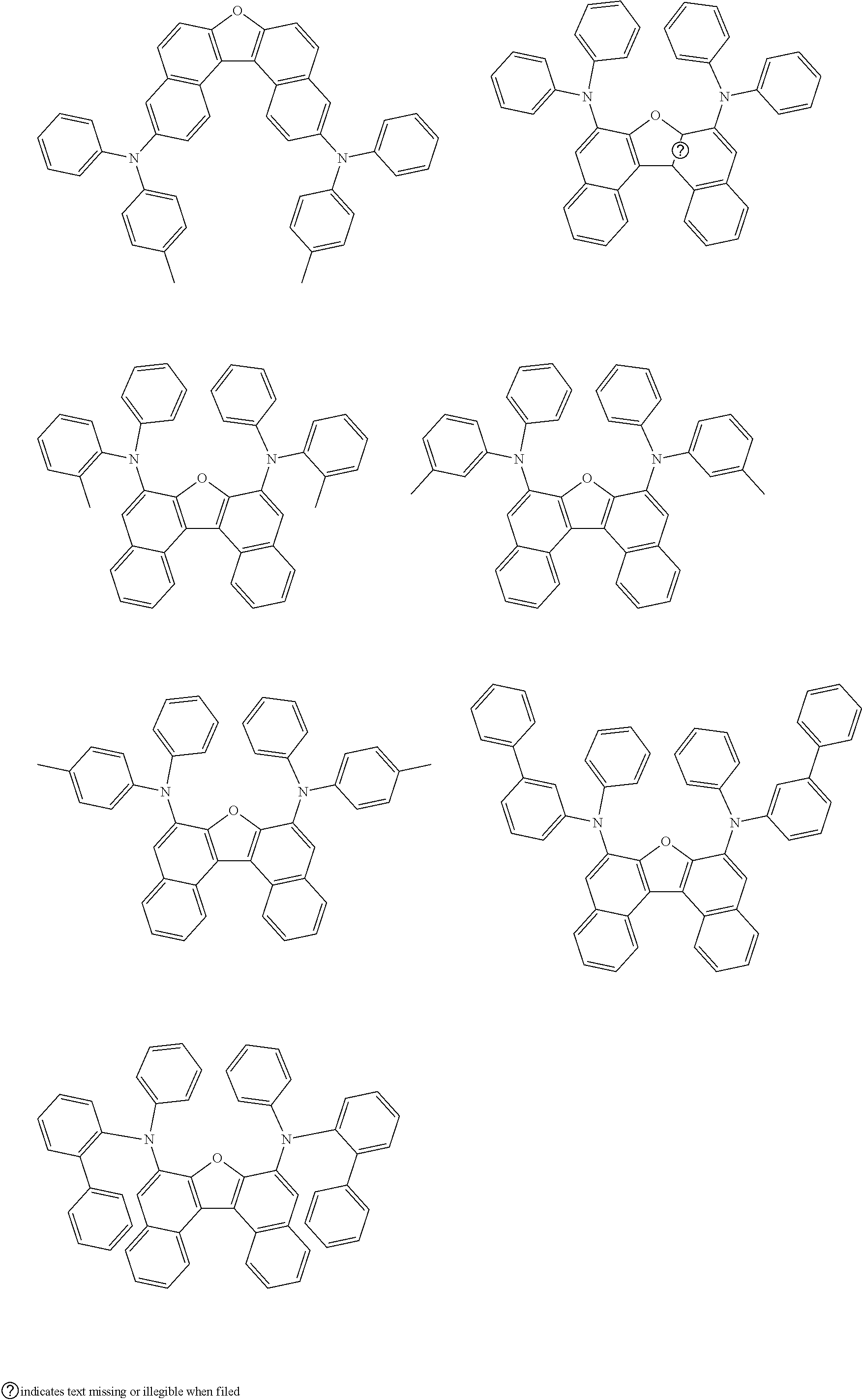

Compound Represented by Formula (9)

A compound represented by the formula (9) will be described below.

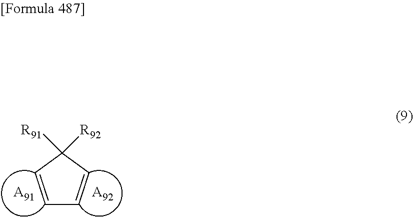

In the formula (9): A₉₁ ring and A₉₂ ring are each independently a substituted or unsubstituted aromatic hydrocarbon ring having 6 to 50 ring carbon atoms, or a substituted or unsubstituted heterocycle having 5 to 50 ring atoms; and at least one ring selected from the group consisting of A₉₁ ring and A₉₂ ring is bonded to * in a structure represented by a formula (92).

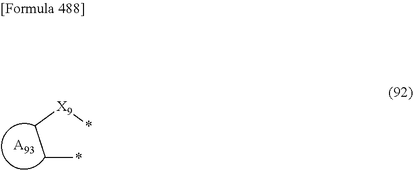

In the formula (92): A93 ring is a substituted or unsubstituted aromatic hydrocarbon ring having 6 to 50 ring carbon atoms, or a substituted or unsubstituted heterocycle having 5 to 50 ring atoms;
X₉ is NR₉₃, C(R₉₄)(R₉₅), Si(R₉₆)(R₉₇), Ge(R₉₈)(R₉₉), an oxygen atom, a sulfur atom, or a selenium atom;
R₉₁ and R₉₂ are mutually bonded to form a substituted or unsubstituted monocyclic ring, mutually bonded to form a substituted or unsubstituted fused ring, or not mutually bonded; and
R₉₁ and R₉₂ forming neither the substituted or unsubstituted monocyclic ring nor the substituted or unsubstituted fused ring, and R₉₃ to R₉₉ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group Represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

At least one ring selected from the group consisting of A₉₁ ring and A₉₂ ring is bonded to a bond * of a structure represented by the formula (92). In other words, the ring-forming carbon atoms of the aromatic hydrocarbon ring or the ring atoms of the heterocycle of the A₉₁ ring in an exemplary embodiment are bonded to the bonds * in a structure represented by the formula (92). Further, the ring-forming carbon atoms of the aromatic hydrocarbon ring or the ring atoms of the heterocycle of the A₉₂ ring in an exemplary embodiment are bonded to the bonds * in a structure represented by the formula (92).

In an exemplary embodiment, a group represented by a formula (93) below is bonded to one or both of the A₉₁ ring and A₉₂ ring.

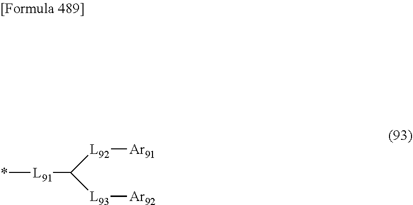

In the formula (93): Ar₉₁ and Ar₉₂ are each independently a substituted or unsubstituted aryl group having 6 to 50 ring carbon atoms, or a substituted or unsubstituted heterocyclic group having 5 to 50 ring atoms;
L₉₁ to L₉₃ are each independently a single bond, a substituted or unsubstituted arylene group having 6 to 30 ring carbon atoms, a substituted or unsubstituted divalent heterocyclic group having 5 to 30 ring atoms, or a divalent linking group formed by bonding two, three or four groups selected from the group consisting of a substituted or unsubstituted arylene group having 6 to 30 ring carbon atoms and a substituted or unsubstituted divalent heterocyclic group having 5 to 30 ring atoms; and
* in the formula (93) represents a bonding position to one of the A₉₁ ring and the A₉₂ ring.

In an exemplary embodiment, in addition to the A₉₁ ring, the ring-forming carbon atoms of the aromatic hydrocarbon ring or the ring atoms of the heterocycle of the A₉₂ ring are bonded to the bonds* in a structure represented by the formula (92). In this case, the moieties represented by the formula (92) may be mutually the same or different.

In an exemplary embodiment, R₉₁ and R₉₂ are each independently a substituted or unsubstituted aryl group having 6 to 50 ring carbon atoms.

In an exemplary embodiment, R₉₁ and R₉₂ are mutually bonded to form a fluorene structure.

In an exemplary embodiment, the rings A₉₁ and A₉₂ are each independently a substituted or unsubstituted aromatic hydrocarbon ring having 6 to 50 ring carbon atoms, example of which is a substituted or unsubstituted benzene ring.

In an exemplary embodiment, the ring A₉₃ is a substituted or unsubstituted aromatic hydrocarbon ring having 6 to 50 ring carbon atoms, example of which is a substituted or unsubstituted benzene ring.

In an exemplary embodiment, X₉ is an oxygen atom or a sulfur atom.

Specific examples of a compound represented by the formula (9) include compounds shown below.

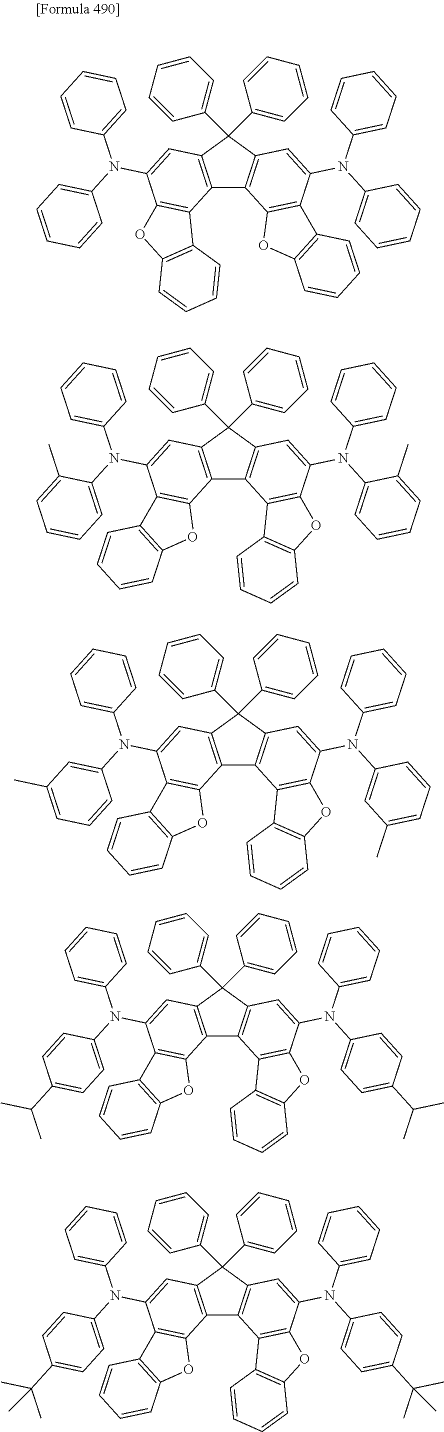

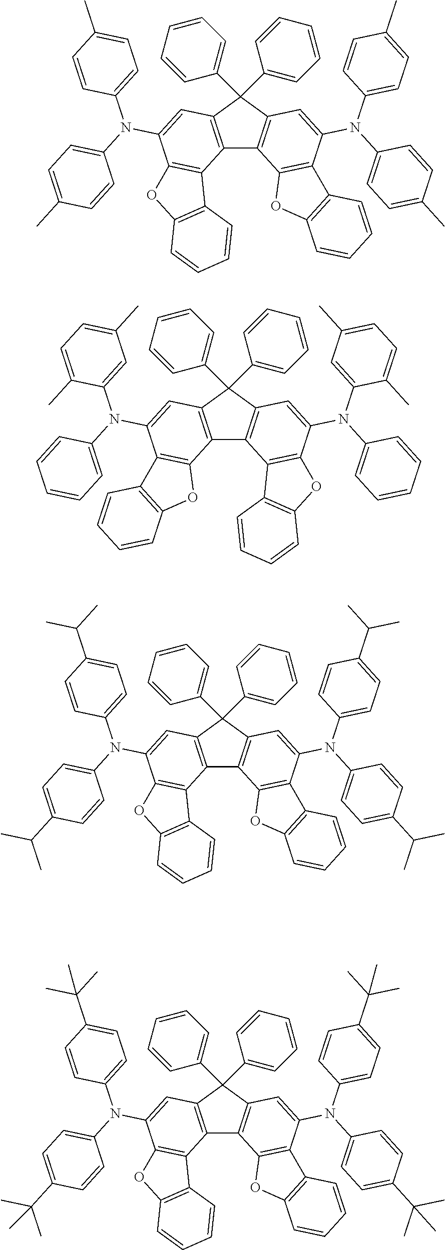

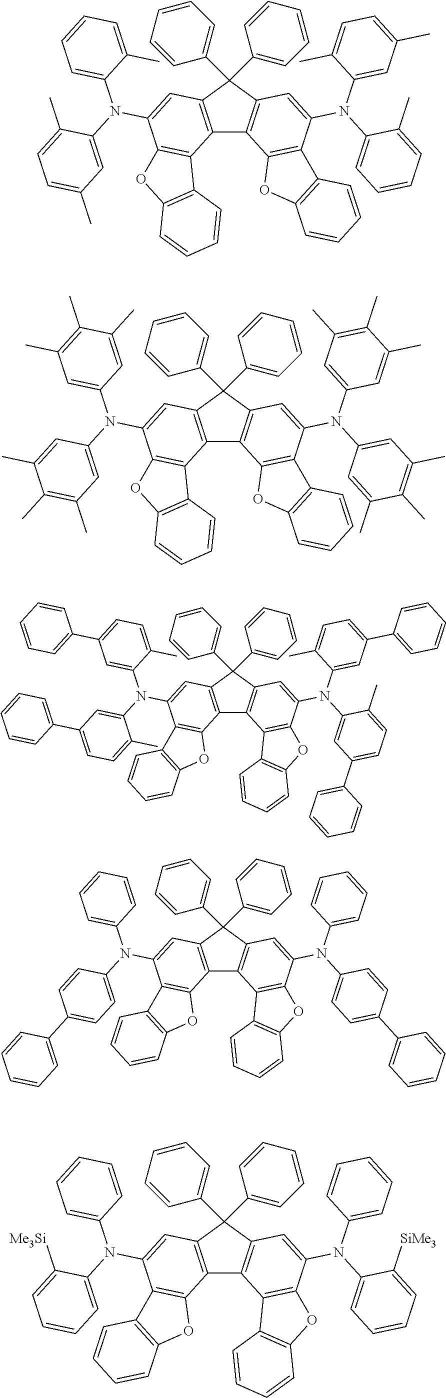

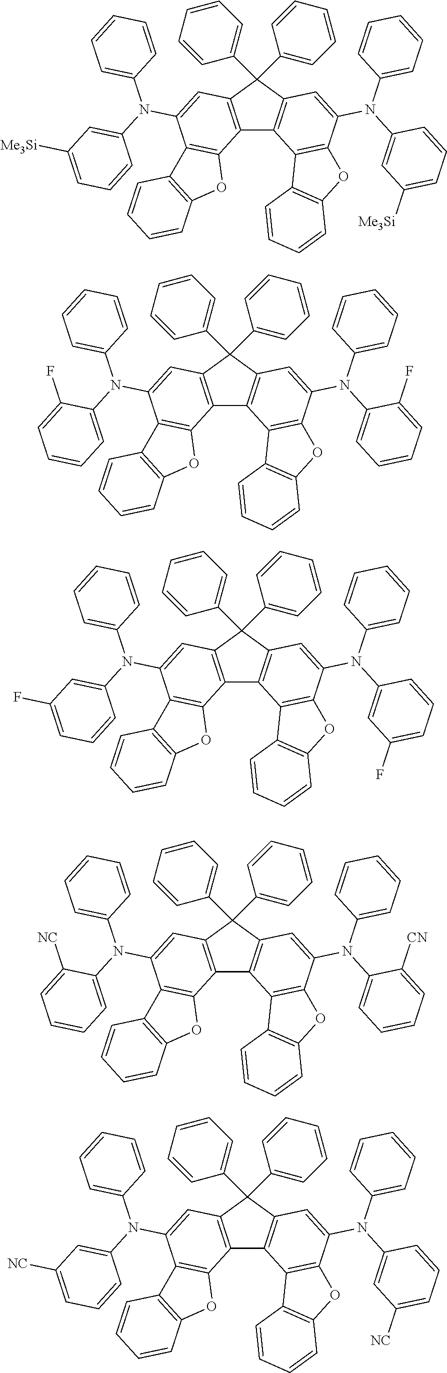

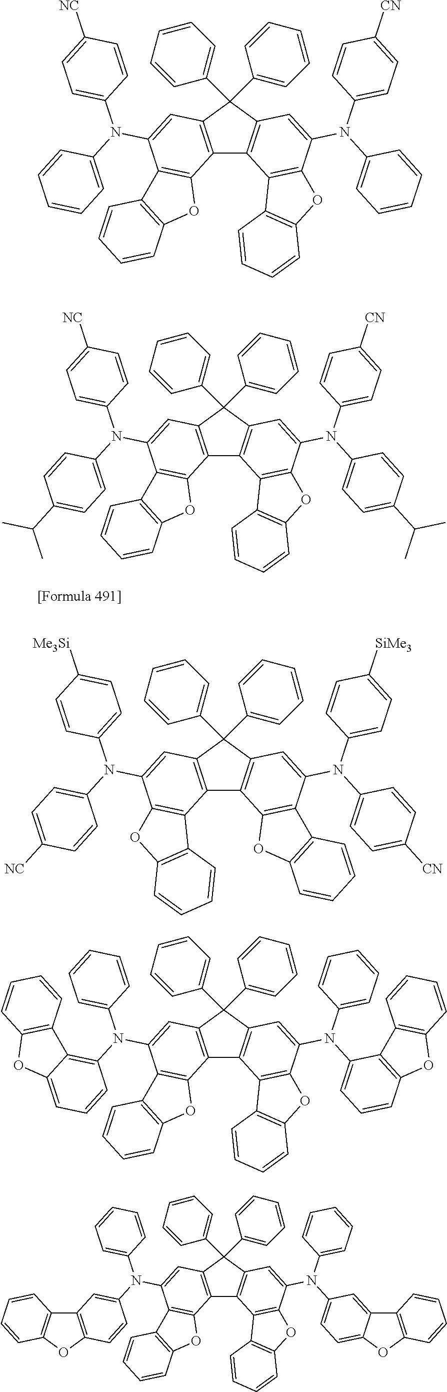

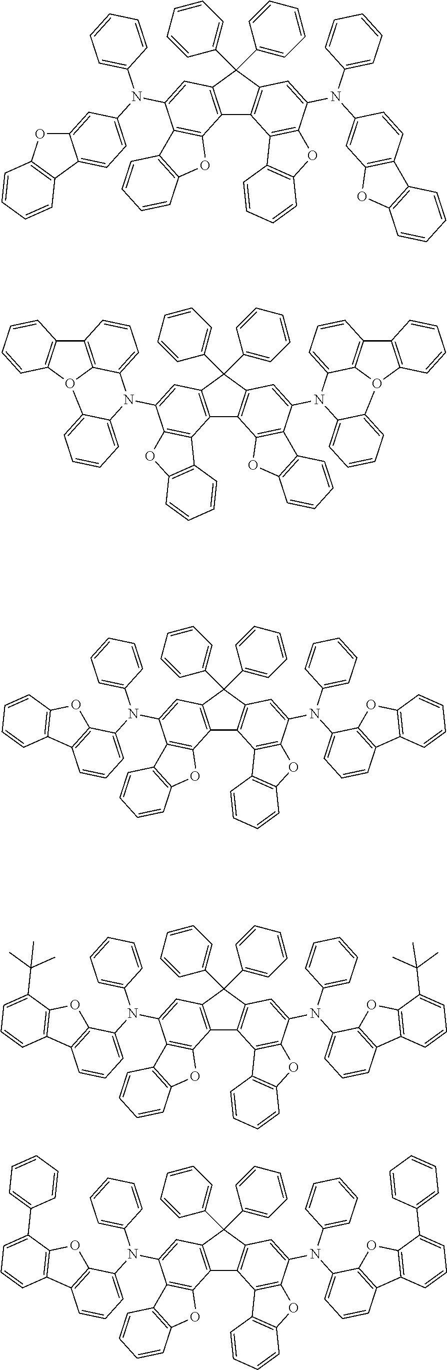

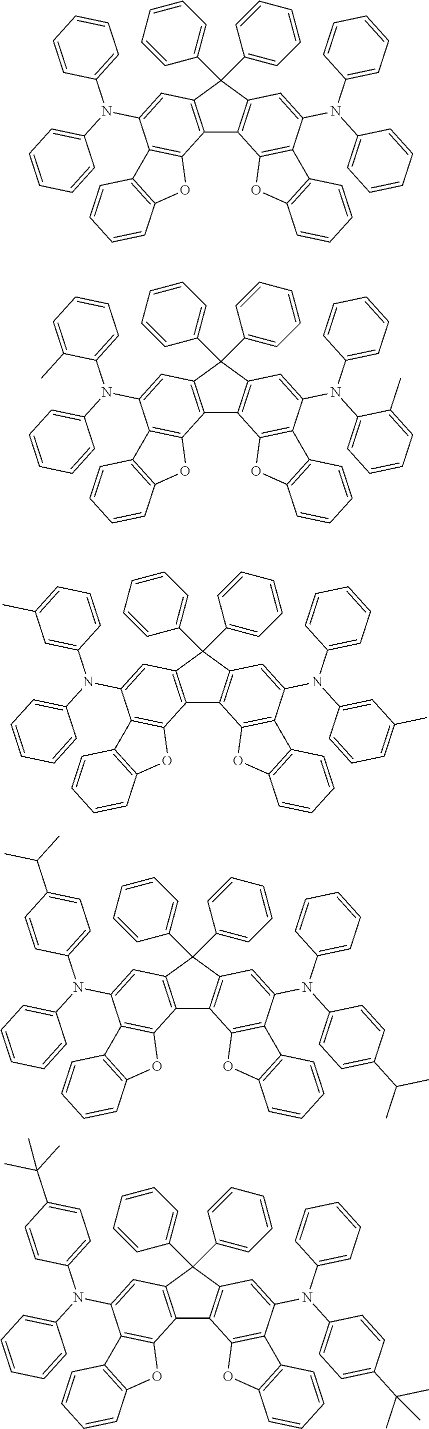

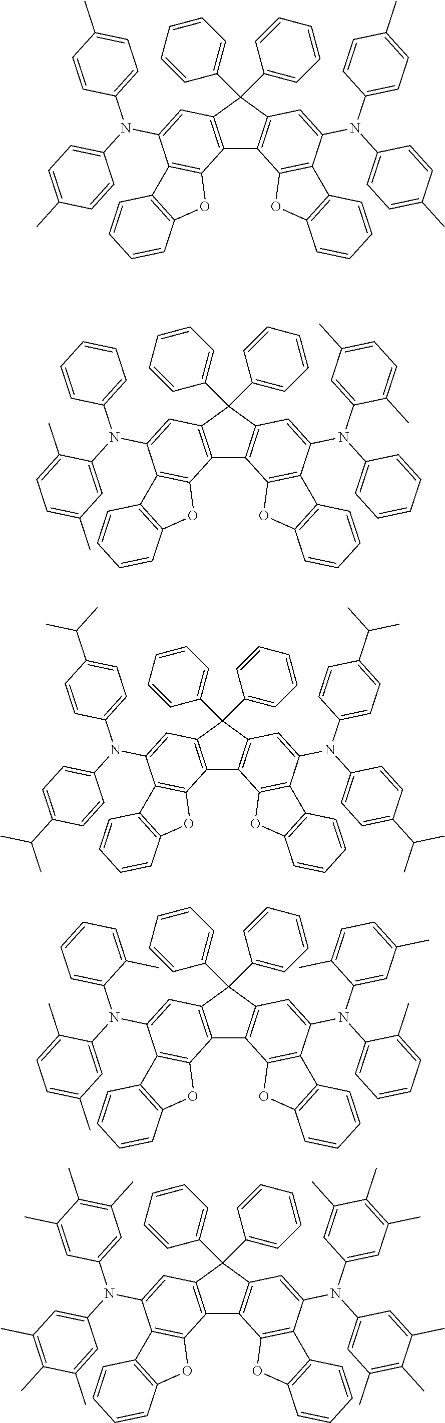

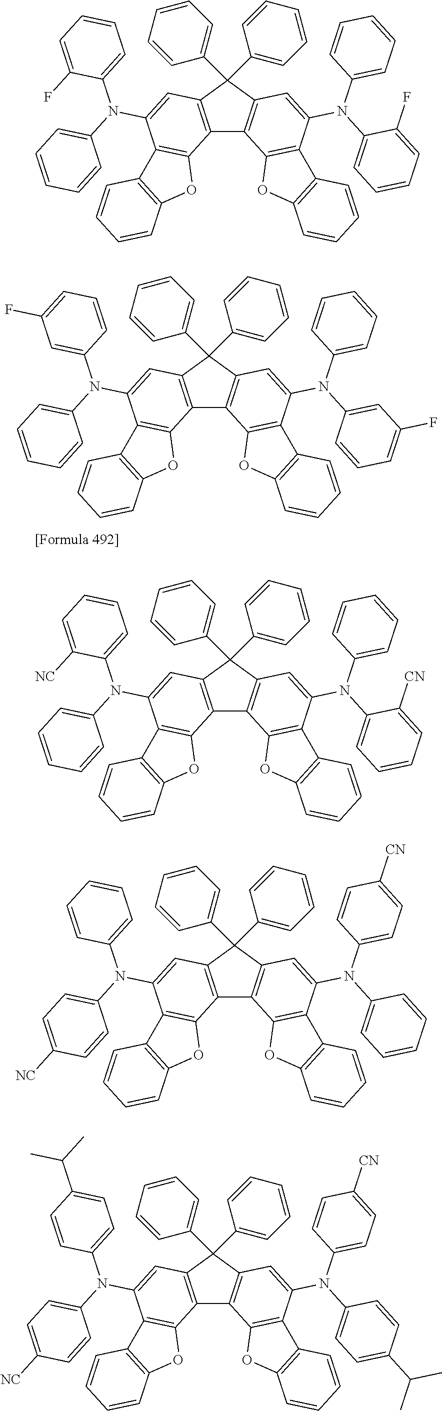

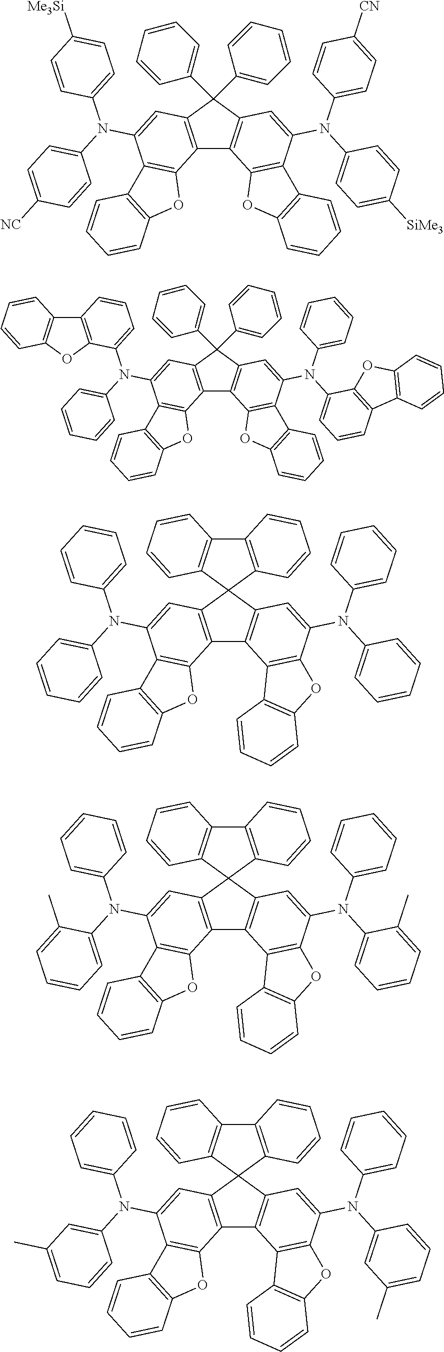

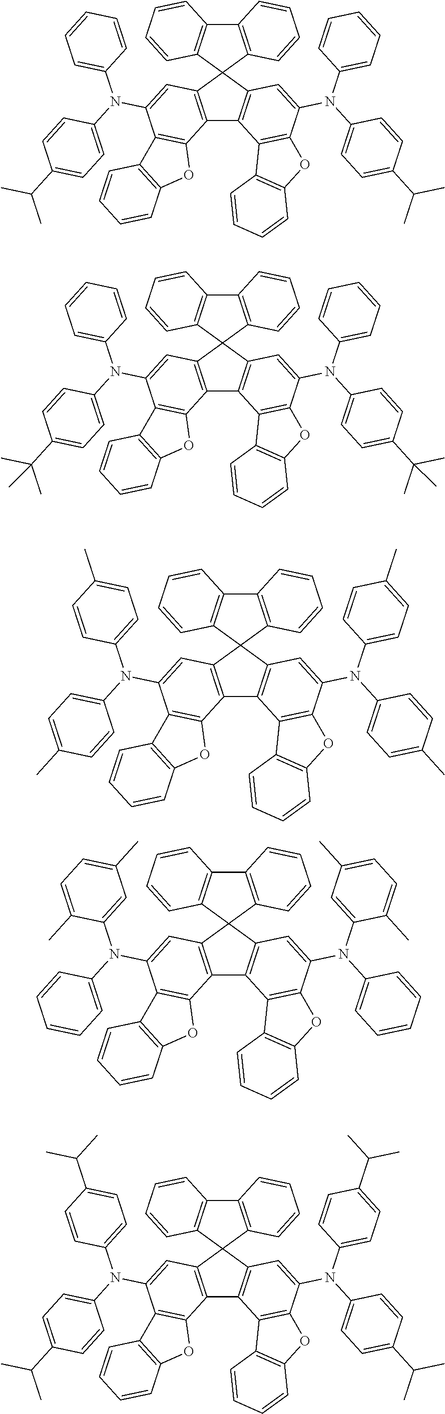

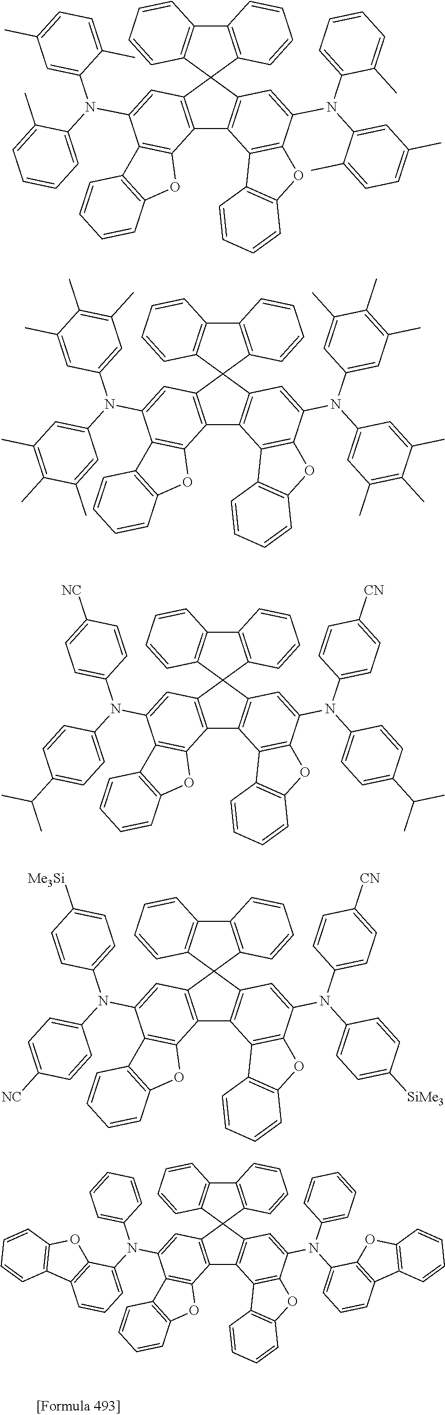

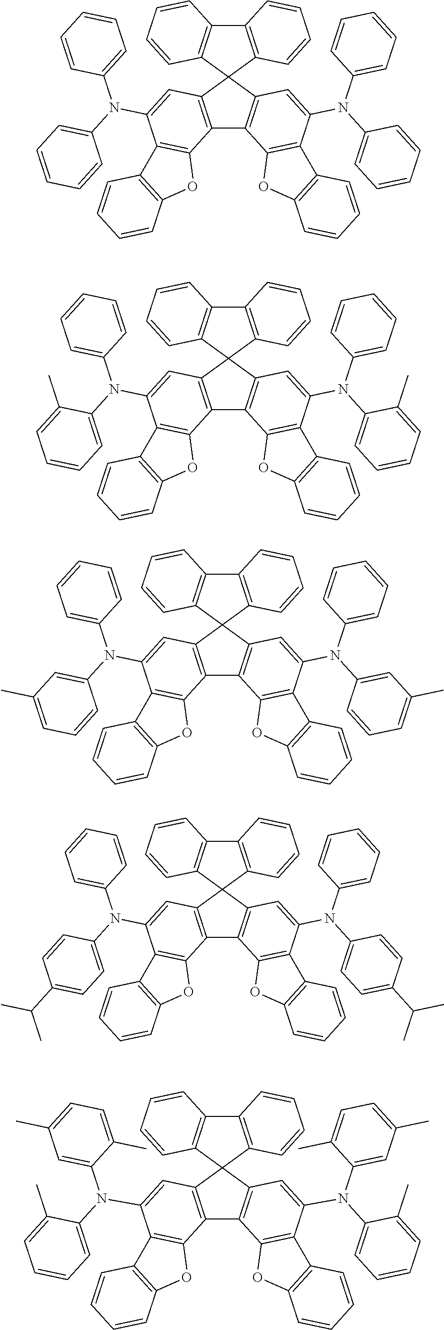

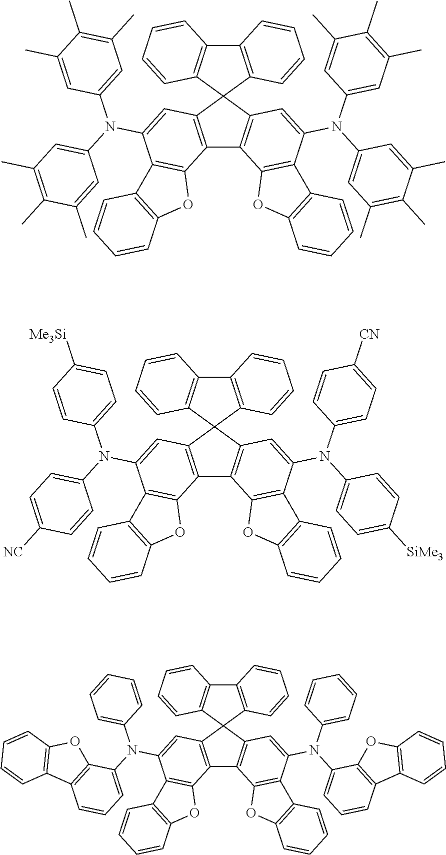

Compound Represented by Formula (10)

A compound represented by the formula (10) will be described below.

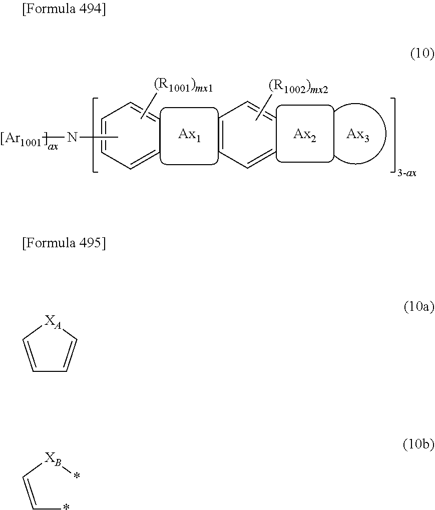

In the formula (10):
Ax₁ ring is a ring represented by the formula (10a) and fused with adjacent ring(s) at any position(s);
Ax₂ ring is a ring represented by the formula (10b) and fused with adjacent ring(s) at any position(s);
two * in the formula (10b) are bonded to Ax₃ ring at any position(s);
X_{A} and X_{B} are each independently C(R₁₀₀₃)(R₁₀₀₄), Si(R₁₀₀₅)(R₁₀₀₆), an oxygen atom or a sulfur atom;
Ax₃ ring is a substituted or unsubstituted aromatic hydrocarbon ring having 6 to 50 ring carbon atoms, or a substituted or unsubstituted heterocycle having 5 to 50 ring atoms;
Ar₁₀₀₁ is a substituted or unsubstituted aryl group having 6 to 50 ring carbon atoms, or a substituted or unsubstituted heterocyclic group having 5 to 50 ring atoms;
R₁₀₀₁ to R₁₀₀₆ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group Represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
mx1 is 3, mx2 is 2;
a plurality of R₁₀₀₁ are mutually the same or different;
a plurality of R₁₀₀₂ are mutually the same or different;
ax is 0, 1, or 2;
when ax is 0 or 1, the structures enclosed by brackets indicated by “3-ax” are mutually the same or different; and
when ax is 2, a plurality of Ar₁₀₀₁ are mutually the same or different.

In an exemplary embodiment, Ar₁₀₀₁ is a substituted or unsubstituted aryl group having 6 to 50 ring carbon atoms.

In an exemplary embodiment, Ax₃ ring is a substituted or unsubstituted aromatic hydrocarbon ring having 6 to 50 ring carbon atoms, example of which is a substituted or unsubstituted benzene ring, a substituted or unsubstituted naphthalene ring, or a substituted or unsubstituted anthracene ring.

In an exemplary embodiment, R₁₀₀₃ and R₁₀₀₄ are each independently a substituted or unsubstituted alkyl group having 1 to 50 carbon atoms.

In an exemplary embodiment, ax is 1.

Specific examples of a compound represented by the formula (10) include compounds shown below.

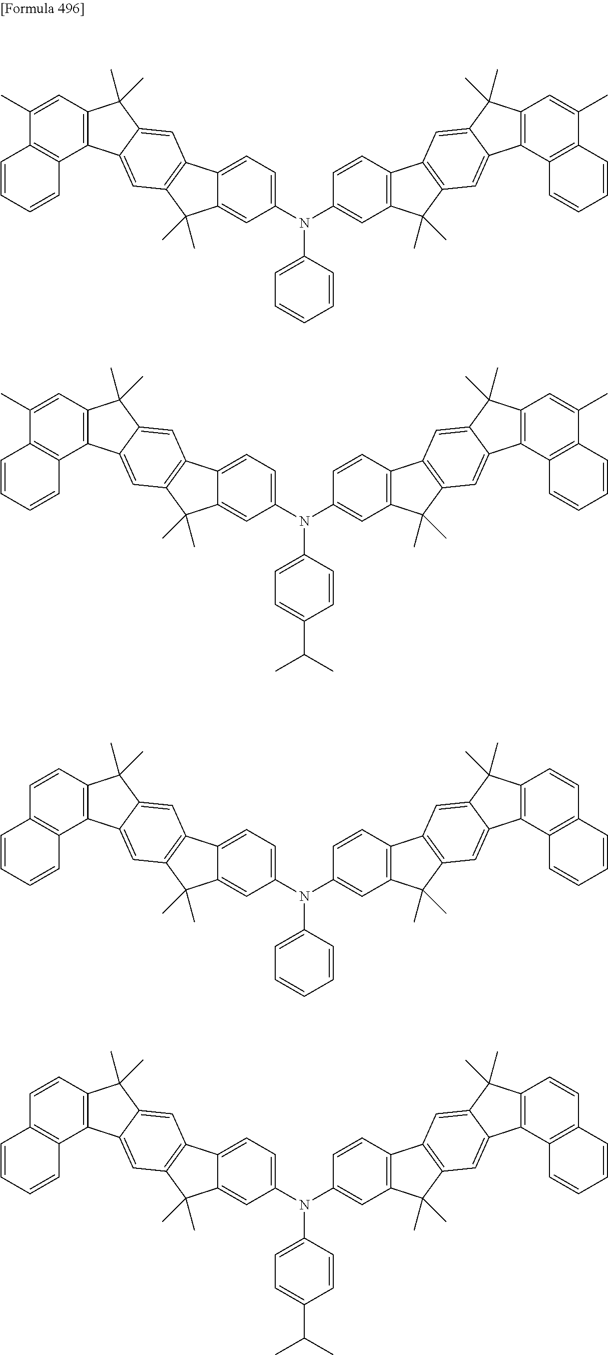

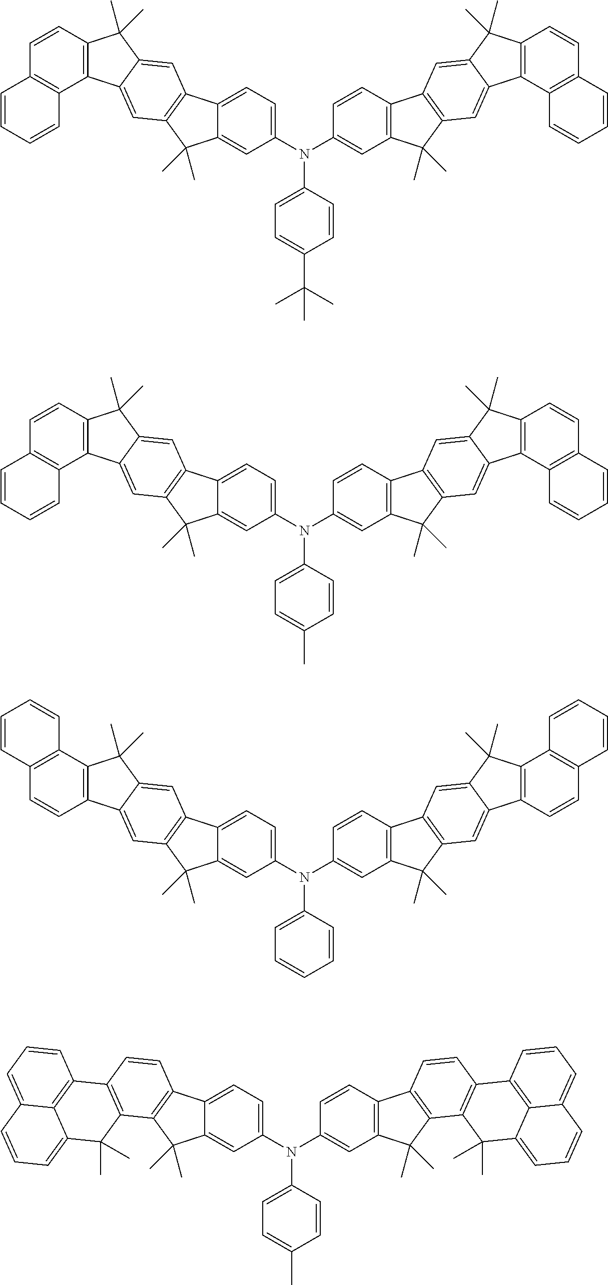 [SMILES: CC(C)(C)c1ccc(N(c2ccc3c(c2)C(C)(C)c2cc4c(cc2-3)C(C)(C)c2ccc3ccccc3c2-4)c2ccc3c(c2)C(C)(C)c2cc4c(cc2-3)C(C)(C)c2ccc3ccccc3c2-4)cc1 ; Cc1ccc(N(c2ccc3c(c2)C(C)(C)c2cc4c(cc2-3)C(C)(C)c2ccc3ccccc3c2-4)c2ccc3c(c2)C(C)(C)c2cc4c(cc2-3)C(C)(C)c2ccc3ccccc3c2-4)cc1 ; CC1(C)c2cc(N(c3ccccc3)c3ccc4c(c3)C(C)(C)c3cc5c(cc3-4)C(C)(C)c3c-5ccc4ccccc34)ccc2-c2cc3c(cc21)-c1ccc2ccccc2c1C3(C)C ; Cc1ccc(N(c2ccc3c(c2)C(C)(C)c2c-3ccc3c2C(C)(C)c2cccc4cccc-3c24)c2ccc3c(c2)C(C)(C)c2c-3ccc3c2C(C)(C)c2cccc4cccc-3c24)cc1]

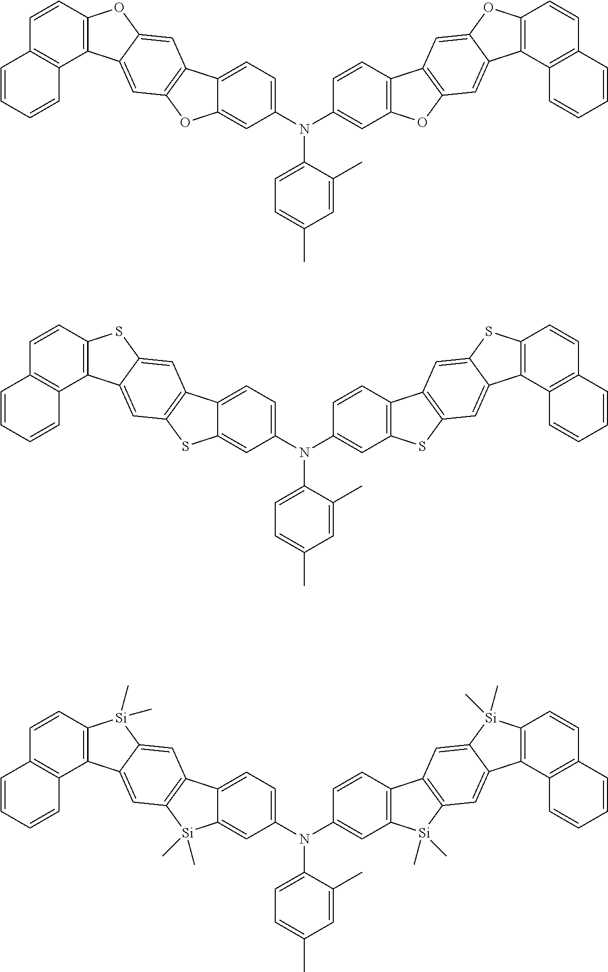 [SMILES: Cc1ccc(N(c2ccc3c(c2)oc2cc4c(cc23)oc2ccc3ccccc3c24)c2ccc3c(c2)oc2cc4c(cc23)oc2ccc3ccccc3c24)c(C)c1 ; Cc1ccc(N(c2ccc3c(c2)sc2cc4c(cc23)sc2ccc3ccccc3c24)c2ccc3c(c2)sc2cc4c(cc23)sc2ccc3ccccc3c24)c(C)c1 ; Cc1ccc(N(c2ccc3c(c2)[Si](C)(C)c2cc4c(cc2-3)[Si](C)(C)c2ccc3ccccc3c2-4)c2ccc3c(c2)[Si](C)(C)c2cc4c(cc2-3)[Si](C)(C)c2ccc3ccccc3c2-4)c(C)c1]

In an exemplary embodiment, the emitting layer contains, as at least one of the third compound or the fourth compound, at least one compound selected from the group consisting of a compound represented by the formula (4), a compound represented by the formula (5), a compound represented by the formula (7), a compound represented by the formula (8), a compound represented by the formula (9), and a compound represented by a formula (63a) below.

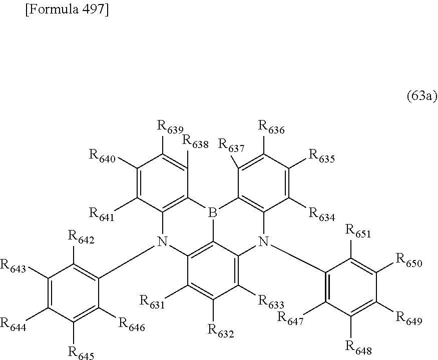

In the formula (63a):
R₆₃₁ is bonded with R₆₄₆ to form a substituted or unsubstituted heterocycle, or not bonded to form no substituted or unsubstituted heterocycle;
R₆₃₃ is bonded with R₆₄₇ to form a substituted or unsubstituted heterocycle, or not bonded to form no substituted or unsubstituted heterocycle;
R₆₃₄ is bonded with R₆₅₁ to form a substituted or unsubstituted heterocycle, or not bonded to form no substituted or unsubstituted heterocycle;
R₆₄₁ is bonded with R₆₄₂ to form a substituted or unsubstituted heterocycle, or not bonded to form no substituted or unsubstituted heterocycle;
at least one combination of adjacent two or more of R₆₃₁ to R₆₅₁ are mutually bonded to form a substituted or unsubstituted monocyclic ring, mutually bonded to form a substituted or unsubstituted fused ring, or not mutually bonded;
R₆₃₁ to R₆₅₁ not forming the substituted or unsubstituted heterocycle, the substituted or unsubstituted monocyclic ring, and the substituted or unsubstituted fused ring are each independently a hydrogen atom, a halogen atom, a cyano group, a nitro group, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group Represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms; and
at least one of R₆₃₁ to R₆₅₁ not forming the substituted or unsubstituted heterocycle, the substituted or unsubstituted monocyclic ring, and the substituted or unsubstituted fused ring is a halogen atom, a cyano group, a nitro group, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group Represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In an exemplary embodiment, a compound represented by the formula (4) is a compound represented by the formula (41-3), the formula (41-4), or the formula (41-5), the A1 ring in the formula (41-5) being a substituted or unsubstituted fused aromatic hydrocarbon ring having 10 to 50 ring carbon atoms, or a substituted or unsubstituted fused heterocycle having 8 to 50 ring atoms.

In an exemplary embodiment, the substituted or unsubstituted fused aromatic hydrocarbon ring having 10 to 50 ring carbon atoms in the formulae (41-3), (41-4) and (41-5) is a substituted or unsubstituted naphthalene ring, a substituted or unsubstituted anthracene ring, or a substituted or unsubstituted fluorene ring; and
the substituted or unsubstituted fused heterocycle having 8 to 50 ring atoms is a substituted or unsubstituted dibenzofuran ring, a substituted or unsubstituted carbazole ring, or a substituted or unsubstituted dibenzothiophene ring.

In an exemplary embodiment, the substituted or unsubstituted fused aromatic hydrocarbon ring having 10 to 50 ring carbon atoms in the formula (41-3), (41-4) or (41-5) is a substituted or unsubstituted naphthalene ring, or a substituted or unsubstituted fluorene ring; and
the substituted or unsubstituted fused heterocycle having 8 to 50 ring atoms is a substituted or unsubstituted dibenzofuran ring, a substituted or unsubstituted carbazole ring, or a substituted or unsubstituted dibenzothiophene ring.

In an exemplary embodiment, a compound represented by the formula (4) is selected from the group consisting of a compound represented by a formula (461) below, a compound represented by a formula (462) below, a compound represented by a formula (463) below, a compound represented by a formula (464) below, a compound represented by a formula (465) below, a compound represented by a formula (466) below, and a compound represented by a formula (467) below.

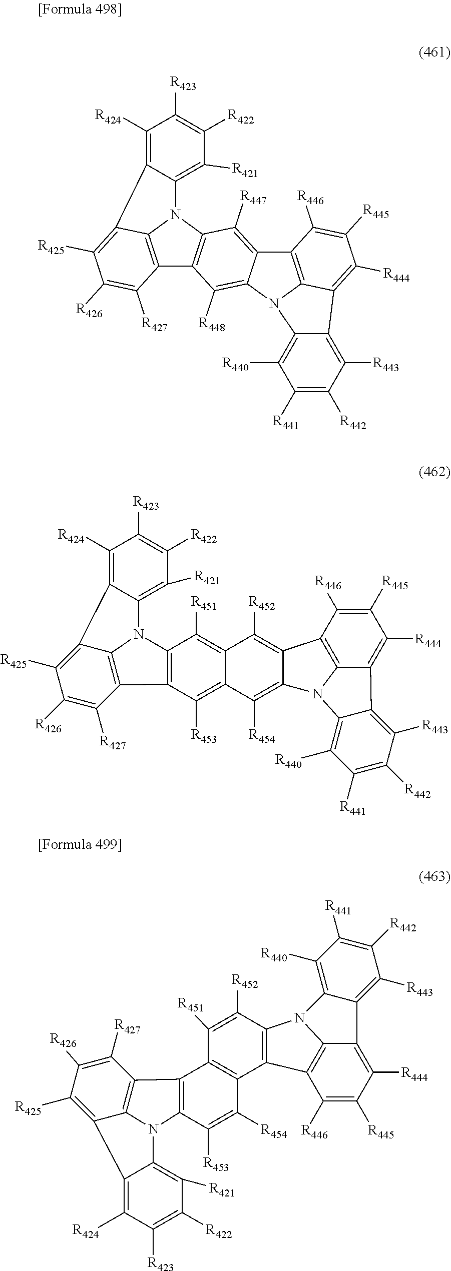

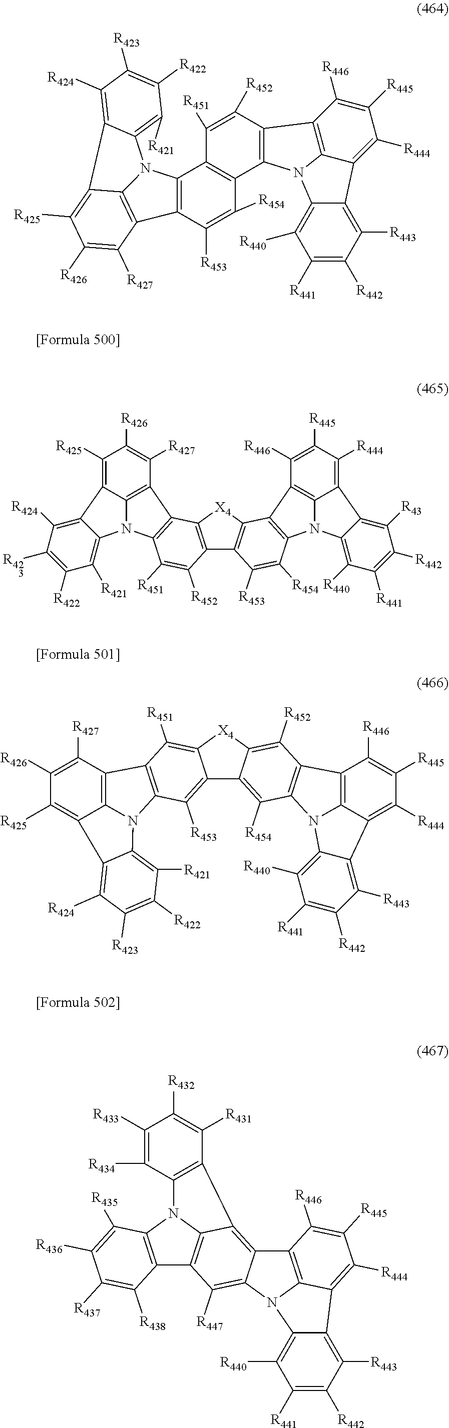

In the formulae (461) to (467): at least one combination of adjacent two or more of R₄₂₁ to R₄₂₇, R₄₃₁ to R₄₃₆, R₄₄₀ to R₄₄₅, and R₄₅₁ to R₄₅₄ are mutually bonded to form a substituted or unsubstituted monocyclic ring, mutually bonded to form a substituted or unsubstituted fused ring, or not mutually bonded;
R₄₂₁ to R₄₂₇, R₄₃₁ to R₄₃₆, R₄₄₀ to R₄₄₅ and R₄₅₁ to R₄₅₄ forming neither the substituted or unsubstituted monocyclic ring nor the substituted or unsubstituted fused ring, and R₄₃₇ and R₄₃₅ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group Represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
X₄ is an oxygen atom, NR₈₀₁, or C(R₈₀₂)(R₈₀₃);
R₈₀₁, R₈₀₂, and R₈₀₃ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
a substituted or unsubstituted alkyl group having 1 to 50 carbon atoms, or a substituted or unsubstituted aryl group having 6 to 50 ring carbon atoms;
when a plurality of R₈₀₁ are present, the plurality of R₈₀₁ are mutually the same or different;
when a plurality of R₈₀₂ are present, the plurality of R₈₀₂ are mutually the same or different; and
when a plurality of R₈₀₃ are present, the plurality of R₈₀₃ are mutually the same or different.

In an exemplary embodiment, R₄₂₁ to R₄₂₇ and R₄₄₀ to R₄₄₈ are each independently a hydrogen atom, a substituted or unsubstituted aryl group having 6 to 50 ring carbon atoms, or a substituted or unsubstituted heterocyclic group having 5 to 50 ring atoms.

In an exemplary embodiment, R₄₂₁ to R₄₂₇ and R₄₄₀ to R₄₄₇ are each independently selected from the group consisting of a hydrogen atom, a substituted or unsubstituted aryl group having 6 to 18 ring carbon atoms, and a substituted or unsubstituted heterocyclic group having 5 to 18 ring atoms.

In an exemplary embodiment, a compound represented by the formula (41-3) is a compound represented by a formula (41-3-1) below.

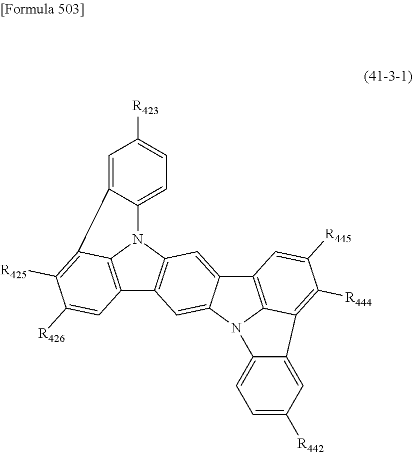

In the formula (41-3-1), R₄₂₃, R₄₂₅, R₄₂₆, R₄₄₂, R₄₄₄ and R₄₄₅ each independently represent the same as R₄₂₃, R₄₂₅, R₄₂₆, R₄₄₂, R₄₄₄ and R₄₄₅ in the formula (41-3).

In an exemplary embodiment, a compound represented by the formula (41-3) is a compound represented by a formula (41-3-2) below.

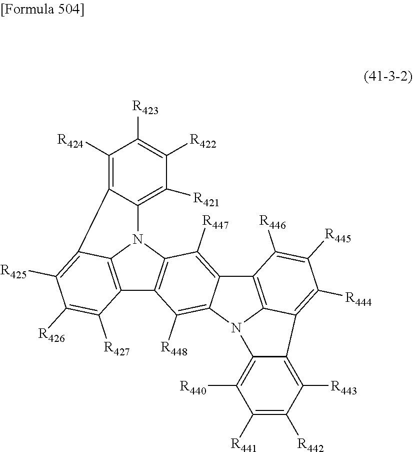

In the formula (41-3-2), R₄₂₁ to R₄₂₇ and R₄₄₀ to R₄₄₈ each independently represent the same as R₄₂₁ to R₄₂₇ and R₄₄₀ to R₄₄₈ in the formula (41-3); and
at least one of R₄₂₁ to R₄₂₇ or R₄₄₀ to R₄₄₆ is a group represented by —N(R₉₀₆)(R₉₀₇).

In an exemplary embodiment, two of R₄₂₁ to R₄₂₇ and R₄₄₀ to R₄₄₆ in the formula (41-3-2) are each a group represented by —N(R₉₀₆)(R₉₀₇).

In an exemplary embodiment, a compound represented by the formula (41-3-2) is a compound represented by a formula (41-3-3) below.

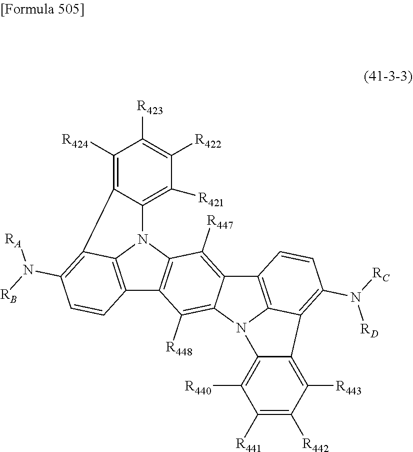

In the formula (41-3-3), R₄₂₁ to R₄₂₄, R₄₄₀ to R₄₄₃, R₄₄₇, and R₄₄₈ each independently represent the same as R₄₂₁ to R₄₂₄, R₄₄₀ to R₄₄₃, R₄₄₇, and R₄₄₈ in the formula (41-3); and
R_{A}, R_{B}, R_{C}, and R_{D} are each independently a substituted or unsubstituted aryl group having 6 to 18 ring carbon atoms, or a substituted or unsubstituted heterocyclic group having 5 to 18 ring atoms.

In an exemplary embodiment, a compound represented by the formula (41-3-3) is a compound represented by a formula (41-3-4) below.

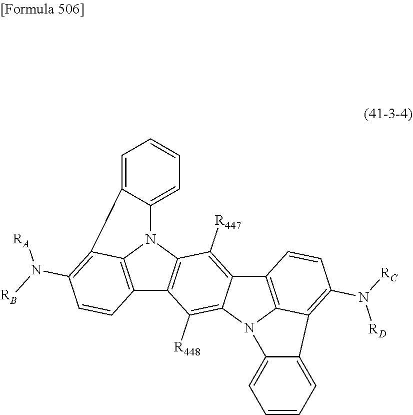

In the formula (41-3-4), R₄₄₇, R₄₄₈, R_{A}, R_{B}, R_{C} and R_{D} each independently represent the same as R₄₄₇, R₄₄₈, R_{A}, R_{B}, R_{C} and R_{D} in the formula (41-3-3).

In an exemplary embodiment, R_{A}, R_{B}, R_{C}, and R_{D} are each independently a substituted or unsubstituted aryl group having 6 to 18 ring carbon atoms.

In an exemplary embodiment, R_{A}, R_{B}, R_{C}, and R_{D} are each independently a substituted or unsubstituted phenyl group.

In an exemplary embodiment, R₄₄₇ and R₄₄₈ are each a hydrogen atom.

In an exemplary embodiment, a substituent for “substituted or unsubstituted” group in each of the formulae is an unsubstituted alkyl group having 1 to 50 carbon atoms, an unsubstituted alkenyl group having 2 to 50 carbon atoms, an unsubstituted alkynyl group having 2 to 50 carbon atoms, an unsubstituted cycloalkyl group having 3 to 50 ring carbon atoms, —Si(R₉₀₁ₐ)(R₉₀₂ₐ)(R₉₀₃ₐ), —O—(R₉₀₄ₐ), —S—(R₉₀₅ₐ), —N(R₉₀₆ₐ)(R₉₀₇ₐ), a halogen atom, a cyano group, a nitro group, an unsubstituted aryl group having 6 to 50 ring carbon atoms, or an unsubstituted heterocyclic group having 5 to 50 ring atoms;
R₉₀₁ₐ to R₉₀₇ₐ are each independently a hydrogen atom, an unsubstituted alkyl group having 1 to 50 carbon atoms, an unsubstituted aryl group having 6 to 50 ring carbon atoms, or an unsubstituted heterocyclic group having 5 to 50 ring atoms;
when two or more R₉₀₁ₐ are present, the two or more R₉₀₁ₐ are mutually the same or different;
when two or more R₉₀₂ₐ are present, the two or more R₉₀₂ₐ are mutually the same or different;
when two or more R₉₀₃ₐ are present, the two or more R₉₀₃ₐ are mutually the same or different;
when two or more R₉₀₄ₐ are present, the two or more R₉₀₄ₐ are mutually the same or different;
when two or more R₉₀₅ₐ are present, the two or more R₉₀₅ₐ are mutually the same or different;
when two or more R₉₀₅ₐ are present, the two or more R₉₀₅ₐ are mutually the same or different; and
when two or more R₉₀₇ₐ are present, the two or more R₉₀₇ₐ are mutually the same or different.

In an exemplary embodiment, a substituent for “substituted or unsubstituted” group in each of the formulae is an unsubstituted alkyl group having 1 to 50 carbon atoms, an unsubstituted aryl group having 6 to 50 ring carbon atoms, or an unsubstituted heterocyclic group having 5 to 50 ring atoms.

In an exemplary embodiment, a substituent for “substituted or unsubstituted” group in each of the formulae is an unsubstituted alkyl group having 1 to 18 carbon atoms, an unsubstituted aryl group having 6 to 18 ring carbon atoms, or an unsubstituted heterocyclic group having 5 to 18 ring atoms.

In the organic EL device according to the exemplary embodiment, it is preferable that the first emitting layer further contains the third compound that fluoresces and the third compound is a compound emitting a light having a main peak wavelength in a range from 430 nm to 480 nm.

In the organic EL device according to the exemplary embodiment, it is preferable that the second emitting layer further contains the fourth compound that fluoresces and the fourth compound is a compound emitting a light having a main peak wavelength in a range from 430 nm to 480 nm.

A measurement method of a main peak wavelength of the compound is as follows. A toluene solution of a measurement target compound at a concentration ranging from 10⁻⁶ mol/L to 10⁻⁵ mol/L is prepared and put in a quartz cell. An emission spectrum (ordinate axis: emission intensity, abscissa axis: wavelength) of the thus-obtained sample is measured at a normal temperature (300K). The emission spectrum is measurable using a spectrophotometer (machine name: F-7000) manufactured by Hitachi High-Tech Science Corporation. It should be noted that the machine for measuring the emission spectrum is not limited to the machine used herein.

A peak wavelength of the emission spectrum exhibiting the maximum luminous intensity is defined as a main peak wavelength. It should be noted that the main peak wavelength is sometimes referred to as a fluorescence main peak wavelength (FL-peak) herein.

In a case where the first emitting layer contains the first compound and the third compound in the organic EL device according to the exemplary embodiment, it is preferable that the first compound is a host material (sometimes referred to as a matrix material) and the third compound is a dopant material (sometimes referred to as a guest material, emitter, or luminescent material).

When the first emitting layer of the organic EL device according to the exemplary embodiment contains the first compound and the third compound, a singlet energy S₁(H1) of the first compound and a singlet energy S₁(D3) of the third compound preferably satisfy a relationship of a numerical formula (Numerical Formula 1) below.

S₁(H1)>S₁(D3) (Numerical Formula 1)

In a case where the second emitting layer contains the second compound and the fourth compound in the organic EL device according to the exemplary embodiment, it is preferable that the second compound is a host material (sometimes referred to as a matrix material) and the fourth compound is a dopant material (sometimes referred to as a guest material, emitter, or luminescent material).

When the second emitting layer of the organic EL device according to the exemplary embodiment contains the second compound and the fourth compound, a singlet energy S₁(H2) of the second compound and a singlet energy S₁(D4) of the fourth compound preferably satisfy a relationship of a numerical formula (Numerical Formula 2) below.

S₁(H2)>S₁(D4) (Numerical Formula 2)

Singlet Energy S₁

A method of measuring a singlet energy S₁ with use of a solution (sometimes referred to as a solution method) is exemplified by a method below.

A toluene solution of a measurement target compound at a concentration ranging from 10⁻⁵ mol/L to 10⁻⁴ mol/L is prepared and put in a quartz cell. An absorption spectrum (ordinate axis: absorption intensity, abscissa axis: wavelength) of the thus-obtained sample is measured at a normal temperature (300K). A tangent is drawn to the fall of the absorption spectrum close to the long-wavelength region, and a wavelength value λedge (nm) at an intersection of the tangent and the abscissa axis is assigned to a conversion equation (F2) below to calculate singlet energy.

S₁ [eV]=1239.85/λedge Conversion Equation (F2):

Any device for measuring absorption spectrum is usable. For instance, a spectrophotometer (U3310 manufactured by Hitachi, Ltd.) is usable.

The tangent to the fall of the absorption spectrum close to the long-wavelength region is drawn as follows. While moving on a curve of the absorption spectrum from the local maximum value closest to the long-wavelength region, among the local maximum values of the absorption spectrum, in a long-wavelength direction, a tangent at each point on the curve is checked. An inclination of the tangent is decreased and increased in a repeated manner as the curve falls (i.e., a value of the ordinate axis is decreased). A tangent drawn at a point where the inclination of the curve is the local minimum closest to the long-wavelength region (except when absorbance is 0.1 or less) is defined as the tangent to the fall of the absorption spectrum close to the long-wavelength region.

The local maximum absorbance of 0.2 or less is not counted as the above-mentioned local maximum absorbance closest to the long-wavelength region.

In the organic EL device according to the exemplary embodiment, an electron mobility μH1 of the first compound and an electron mobility μH2 of the second compound also preferably satisfy a relationship of a numerical formula (Numerical Formula 3) below.

μH2>μH1 (Numerical Formula 3)

When the first compound and the second compound satisfy the relationship of the numerical formula (Numerical Formula 3), a recombination ability of holes and electrons in the first emitting layer is improved.

The electron mobility can be measured according to impedance spectroscopy.

A measurement target layer having a thickness in a range from 100 nm to 200 nm is held between the anode and the cathode, to which a small alternating voltage of 100 mV or less is applied while a bias DC voltage is applied. A value of an alternating current (absolute value and phase) which flows at this time is measured. This measurement is performed while changing a frequency of the alternating voltage, and complex impedance (Z) is calculated from the current value and the voltage value. A frequency dependency of the imaginary part (ImM) of the modulus M=iωZ (i: imaginary unit, w: angular frequency) is obtained. The reciprocal number of a frequency ω at which the ImM becomes the maximum is defined as a response time of electrons carried in the measurement target layer. The electron mobility is calculated by the following equation.

Electron Mobility=(Film Thickness of Measurement Target Layer)²/(Response Time-Voltage)

The first emitting layer and the second emitting layer preferably do not contain a phosphorescent material (dopant material).

The first emitting layer and the second emitting layer preferably do not contain a heavy metal complex and a phosphorescent rare earth metal complex. Examples of the heavy-metal complex herein include iridium complex, osmium complex, and platinum complex.

Further, the first emitting layer and the second emitting layer also preferably do not contain a metal complex.

Film Thickness of Emitting Layer

A film thickness of the emitting layer of the organic EL device according to the exemplary embodiment is preferably in a range from 5 nm to 50 nm, more preferably in a range from 7 nm to 50 nm, further preferably in a range from 10 nm to 50 nm. When the film thickness of the emitting layer is 5 nm or more, the emitting layer is easily formable and chromaticity is easily adjustable. When the film thickness of the emitting layer is 50 nm or less, a rise in the drive voltage is easily reducible.

Content Ratios of Compounds in Emitting Layer

When the first emitting layer contains the first compound and the third compound, a content ratio of each of the first compound and the third compound in the first emitting layer preferably falls, for instance, within a range below.

The content ratio of the first compound is preferably in a range from 80 mass % to 99 mass %, more preferably in a range from 90 mass % to 99 mass %, further preferably in a range from 95 mass % to 99 mass %.

The content ratio of the third compound is preferably in a range from 1 mass % to 10 mass %, more preferably in a range from 1 mass % to 7 mass %, further preferably in a range from 1 mass % to 5 mass %.

The upper limit of the total of the content ratios of the first compound and the third compound in the first emitting layer is 100 mass %.

It is not excluded that the first emitting layer of the exemplary embodiment further contains a material(s) other than the first and third compounds.

The first emitting layer may contain a single type of the first compound or two or more types of the first compound. The first emitting layer may contain a single type of the third compound or two or more types of the third compound.

When the second emitting layer contains the second compound and the fourth compound, a content ratio of each of the second compound and the fourth compound in the second emitting layer preferably falls, for instance, within a range below.

The content ratio of the second compound is preferably in a range from 80 mass % to 99 mass %, more preferably in a range from 90 mass % to 99 mass %, further preferably in a range from 95 mass % to 99 mass %.

The content ratio of the fourth compound is preferably in a range from 1 mass % to 10 mass %, more preferably in a range from 1 mass % to 7 mass %, further preferably in a range from 1 mass % to 5 mass %.

The upper limit of the total of the content ratios of the second compound and the fourth compound in the second emitting layer is 100 mass %.

It is not excluded that the second emitting layer of the exemplary embodiment further contains a material(s) other than the second and fourth compounds.

The second emitting layer may contain a single type of the second compound or two or more types of the second compound. The second emitting layer may contain a single type of the fourth compound or two or more types of the fourth compound.

An arrangement of an organic EL device will be further described below. It should be noted that the reference numerals will be sometimes omitted below.

Substrate

The substrate is used as a support for the organic EL device. For instance, glass, quartz, plastics and the like are usable for the substrate. A flexible substrate is also usable. The flexible substrate is a bendable substrate, which is exemplified by a plastic substrate. Examples of the material for the plastic substrate include polycarbonate, polyarylate, polyethersulfone, polypropylene, polyester, polyvinyl fluoride, polyvinyl chloride, polyimide, and polyethylene naphthalate. Moreover, an inorganic vapor deposition film is also usable.

Anode

Metal, an alloy, an electrically conductive compound, a mixture thereof, or the like having a large work function (specifically, 4.0 eV or more) is preferably used as the anode formed on the substrate. Specific examples of the material include ITO (Indium Tin Oxide), indium oxide-tin oxide containing silicon or silicon oxide, indium oxide-zinc oxide, indium oxide containing tungsten oxide and zinc oxide, and graphene. In addition, gold (Au), platinum (Pt), nickel (Ni), tungsten (W), chrome (Cr), molybdenum (Mo), iron (Fe), cobalt (Co), copper (Cu), palladium (Pd), titanium (Ti), and nitrides of a metal material (e.g., titanium nitride) are usable.

The material is typically formed into a film by a sputtering method. For instance, the indium oxide-zinc oxide can be formed into a film by the sputtering method using a target in which zinc oxide in a range from 1 mass % to 10 mass % is added to indium oxide. Moreover, for instance, the indium oxide containing tungsten oxide and zinc oxide can be formed by the sputtering method using a target in which tungsten oxide in a range from 0.5 mass % to 5 mass % and zinc oxide in a range from 0.1 mass % to 1 mass % are added to indium oxide. In addition, the anode may be formed by a vacuum deposition method, a coating method, an inkjet method, a spin coating method or the like.

Among the organic layers formed on the anode, since the hole injecting layer adjacent to the anode is formed of a composite material into which holes are easily injectable irrespective of the work function of the anode, a material usable as an electrode material (e.g., metal, an alloy, an electroconductive compound, a mixture thereof, and the elements belonging to the group 1 or 2 of the periodic table) is also usable for the anode.

A material having a small work function such as elements belonging to Groups 1 and 2 in the periodic table of the elements, specifically, an alkali metal such as lithium (Li) and cesium (Cs), an alkaline earth metal such as magnesium (Mg), calcium (Ca) and strontium (Sr), alloys (e.g., MgAg and AILi) including the alkali metal or the alkaline earth metal, a rare earth metal such as europium (Eu) and ytterbium (Yb), alloys including the rare earth metal are also usable for the anode. It should be noted that the vacuum deposition method and the sputtering method are usable for forming the anode using the alkali metal, alkaline earth metal and the alloy thereof. Further, when a silver paste is used for the anode, the coating method and the inkjet method are usable.

Cathode

It is preferable to use metal, an alloy, an electroconductive compound, a mixture thereof, or the like having a small work function (specifically, 3.8 eV or less) for the cathode. Examples of the material for the cathode include elements belonging to Groups 1 and 2 in the periodic table of the elements, specifically, the alkali metal such as lithium (Li) and cesium (Cs), the alkaline earth metal such as magnesium (Mg), calcium (Ca) and strontium (Sr), alloys (e.g., MgAg and AILi) including the alkali metal or the alkaline earth metal, the rare earth metal such as europium (Eu) and ytterbium (Yb), and alloys including the rare earth metal.

It should be noted that the vacuum deposition method and the sputtering method are usable for forming the cathode using the alkali metal, alkaline earth metal and the alloy thereof. Further, when a silver paste is used for the cathode, the coating method and the inkjet method are usable.

By providing the electron injecting layer, various conductive materials such as Al, Ag, ITO, graphene, and indium oxide-tin oxide containing silicon or silicon oxide may be used for forming the cathode regardless of the work function. The conductive materials can be formed into a film using the sputtering method, inkjet method, spin coating method and the like.

Hole Injecting Layer

The hole injecting layer is a layer containing a substance exhibiting a high hole injectability. Examples of the substance exhibiting a high hole injectability include molybdenum oxide, titanium oxide, vanadium oxide, rhenium oxide, ruthenium oxide, chrome oxide, zirconium oxide, hafnium oxide, tantalum oxide, silver oxide, tungsten oxide, and manganese oxide.

In addition, the examples of the highly hole-injectable substance further include: an aromatic amine compound, which is a low-molecule organic compound, such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), 4,4′-bis(N-{4-[N′-(3-methylphenyl)-N′-phenylamino]phenyl}-N-phenylamino)biphenyl (abbreviation: DNTPD), 1,3,5-tris[N-(4-diphenylaminophenyl)-N-phenylamino]benzene (abbreviation: DPA3B), 3-[N-(9-phenylcarbazole-3-yl)-N-phenylamino]-9-phenylcarbazole (abbreviation: PCzPCA1), 3,6-bis[N-(9-phenylcarbazole-3-yl)-N-phenylamino]-9-phenylcarbazole (abbreviation: PCzPCA2), and 3-[N-(1-naphthyl)-N-(9-phenylcarbazole-3-yl)amino]-9-phenylcarbazole (abbreviation: PCzPCN1); and dipyrazino[2,3-f:20,30-h]quinoxaline-2,3,6,7,10,11-hexacarbonitrile (HAT-CN).

In addition, a high polymer compound (e.g., oligomer, dendrimer and polymer) is usable as the substance exhibiting a high hole injectability. Examples of the high-molecule compound include poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide](abbreviation: PTPDMA), and poly[N,N′-bis(4-butylphenyl)-N,N′-bis(phenyl)benzidine] (abbreviation: Poly-TPD). Moreover, an acid-added high polymer compound such as poly(3,4-ethylenedioxythiophene)/poly(styrene sulfonic acid) (PEDOT/PSS) and polyaniline/poly(styrene sulfonic acid) (PAni/PSS) are also usable.

Hole Transporting Layer

The hole transporting layer is a layer containing a highly hole-transporting substance. An aromatic amine compound, carbazole derivative, anthracene derivative and the like are usable for the hole transporting layer. Specific examples of a material for the hole transporting layer include 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4-phenyl-4′-(9-phenylfluorene-9-yl)triphenylamine (abbreviation: BAFLP), 4,4′-bis[N-(9,9-dimethylfluorene-2-yl)-N-phenylamino]biphenyl (abbreviation: DFLDPBi), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), and 4,4′-bis[N-(spiro-9,9′-bifluorene-2-yl)-N-phenylamino]biphenyl (abbreviation: BSPB). The above-described substances mostly have a hole mobility of 10⁻⁶ cm²/(V·s) or more.

For the hole transporting layer, a carbazole derivative such as CBP, 9-[4-(N-carbazolyl)]phenyl-10-phenylanthracene (CzPA), and 9-phenyl-3-[4-(10-phenyl-9-anthryl)phenyl]-9H-carbazole (PCzPA) and an anthracene derivative such as t-BuDNA, DNA, and DPAnth may be used. A high polymer compound such as poly(N-vinylcarbazole) (abbreviation: PVK) and poly(4-vinyltriphenylamine) (abbreviation: PVTPA) is also usable.

However, in addition to the above substances, any substance exhibiting a higher hole transportability than an electron transportability may be used. It should be noted that the layer containing the substance exhibiting a high hole transportability may be not only a single layer but also a laminate of two or more layers formed of the above substance(s).

Electron Transporting Layer

The electron transporting layer is a layer containing a highly electron-transporting substance. For the electron transporting layer, 1) a metal complex such as an aluminum complex, beryllium complex, and zinc complex, 2) a hetero aromatic compound such as imidazole derivative, benzimidazole derivative, azine derivative, carbazole derivative, and phenanthroline derivative, and 3) a high polymer compound are usable. Specifically, as a low-molecule organic compound, a metal complex such as Alq, tris(4-methyl-8-quinolinato)aluminum (abbreviation: Almq₃), bis(10-hydroxybenzo[h]quinolinato)beryllium (abbreviation: BeBq₂), BAlq, Znq, ZnPBO and ZnBTZ is usable. In addition to the metal complex, a heteroaromatic compound such as 2-(4-biphenylyl)-5-(4-tert-butylphenyl)-1,3,4-oxadiazole (abbreviation: PBD), 1,3-bis[5-(ptert-butylphenyl)-1,3,4-oxadiazole-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzoxazole-2-yl)stilbene (abbreviation: BzOs) is usable. In the exemplary embodiment, a benzimidazole compound is preferably usable. The above-described substances mostly have an electron mobility of 10⁻⁶ cm²Ns or more. It should be noted that any substance other than the above substance may be used for the electron transporting layer as long as the substance exhibits a higher electron transportability than the hole transportability. The electron transporting layer may be provided in the form of a single layer or a laminate of two or more layers of the above substance(s).

Specific examples of the compound usable for the electron transporting layer include compounds below. It should however be noted that the invention is not limited to the specific examples of the compound.

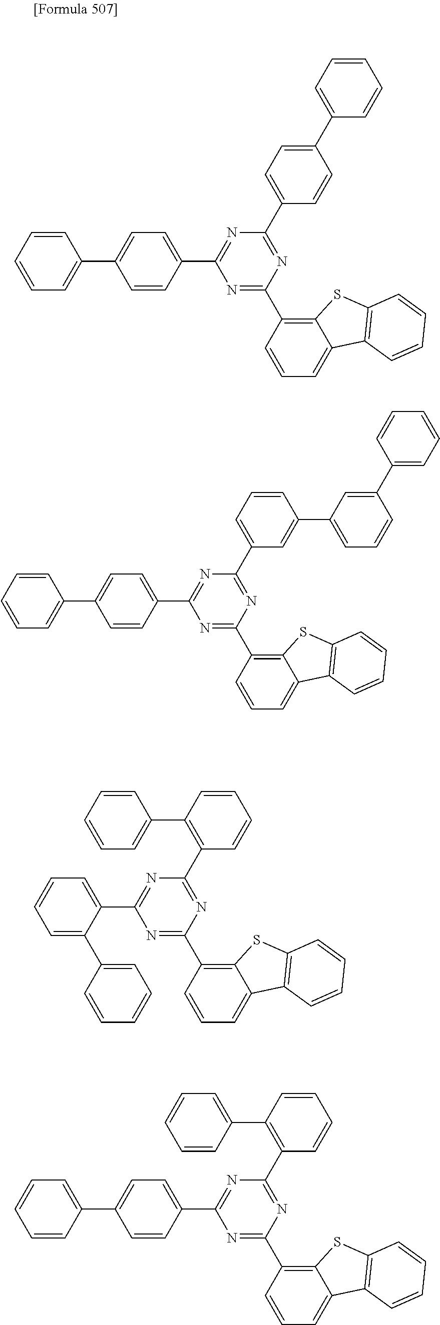 [SMILES: c1ccc(-c2ccc(-c3nc(-c4ccc(-c5ccccc5)cc4)nc(-c4cccc5c4sc4ccccc45)n3)cc2)cc1 ; c1ccc(-c2ccc(-c3nc(-c4cccc(-c5cccc(-c6ccccc6)c5)c4)nc(-c4cccc5c4sc4ccccc45)n3)cc2)cc1 ; c1ccc(-c2ccccc2-c2nc(-c3ccccc3-c3ccccc3)nc(-c3cccc4c3sc3ccccc34)n2)cc1 ; c1ccc(-c2ccc(-c3nc(-c4ccccc4-c4ccccc4)nc(-c4cccc5c4sc4ccccc45)n3)cc2)cc1]

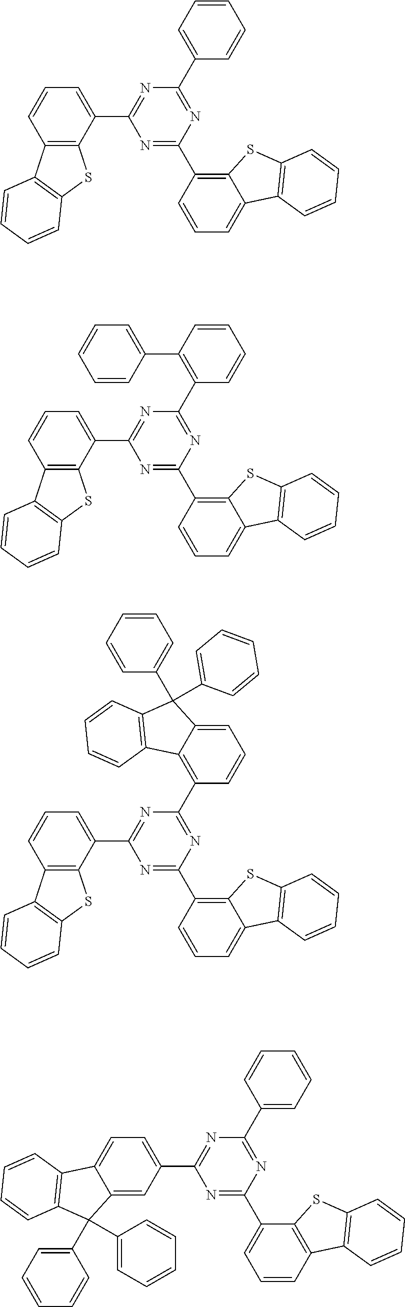 [SMILES: c1ccc(-c2nc(-c3cccc4c3sc3ccccc34)nc(-c3cccc4c3sc3ccccc34)n2)cc1 ; c1ccc(-c2ccccc2-c2nc(-c3cccc4c3sc3ccccc34)nc(-c3cccc4c3sc3ccccc34)n2)cc1 ; c1ccc(C2(c3ccccc3)c3ccccc3-c3c(-c4nc(-c5cccc6c5sc5ccccc56)nc(-c5cccc6c5sc5ccccc56)n4)cccc32)cc1 ; c1ccc(-c2nc(-c3ccc4c(c3)C(c3ccccc3)(c3ccccc3)c3ccccc3-4)nc(-c3cccc4c3sc3ccccc34)n2)cc1]

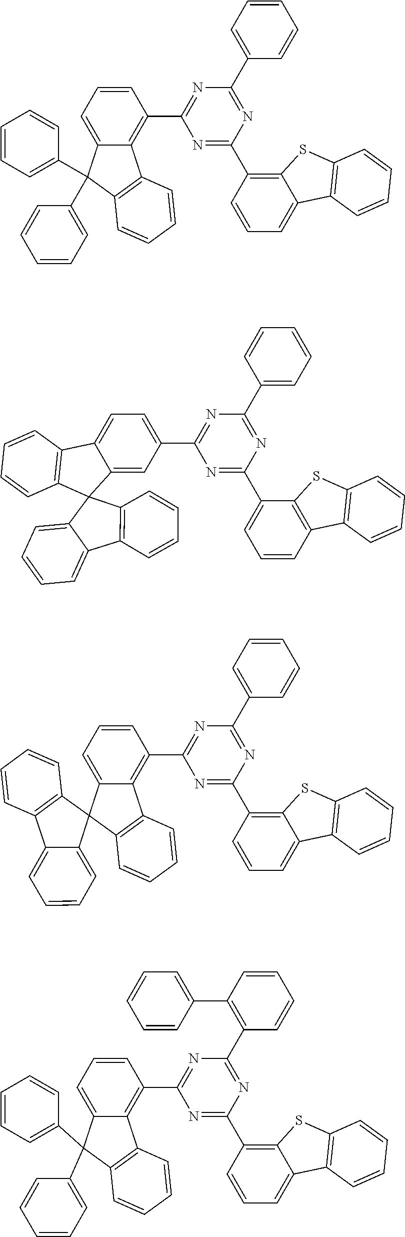 [SMILES: c1ccc(-c2nc(-c3cccc4c3-c3ccccc3C4(c3ccccc3)c3ccccc3)nc(-c3cccc4c3sc3ccccc34)n2)cc1 ; c1ccc(-c2nc(-c3ccc4c(c3)C3(c5ccccc5-c5ccccc53)c3ccccc3-4)nc(-c3cccc4c3sc3ccccc34)n2)cc1 ; c1ccc(-c2nc(-c3cccc4c3-c3ccccc3C43c4ccccc4-c4ccccc43)nc(-c3cccc4c3sc3ccccc34)n2)cc1 ; c1ccc(-c2ccccc2-c2nc(-c3cccc4c3-c3ccccc3C4(c3ccccc3)c3ccccc3)nc(-c3cccc4c3sc3ccccc34)n2)cc1]

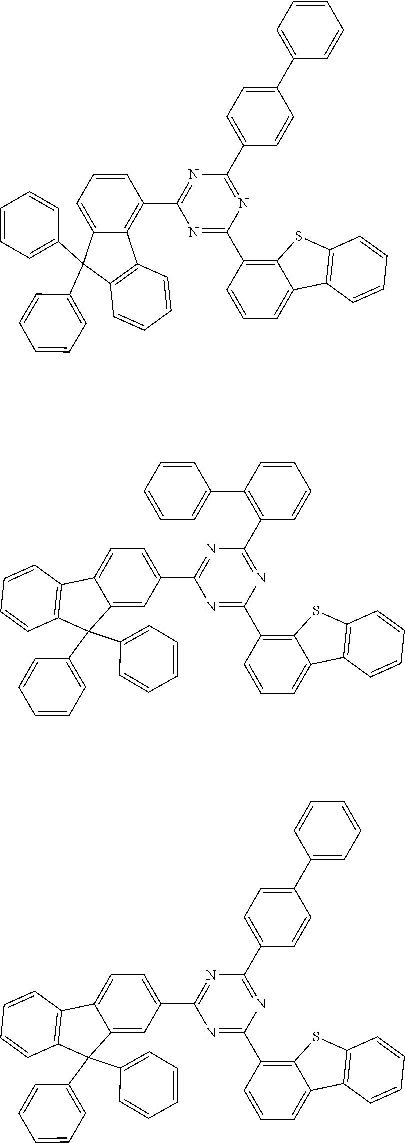 [SMILES: c1ccc(-c2ccc(-c3nc(-c4cccc5c4-c4ccccc4C5(c4ccccc4)c4ccccc4)nc(-c4cccc5c4sc4ccccc45)n3)cc2)cc1 ; c1ccc(-c2ccccc2-c2nc(-c3ccc4c(c3)C(c3ccccc3)(c3ccccc3)c3ccccc3-4)nc(-c3cccc4c3sc3ccccc34)n2)cc1 ; c1ccc(-c2ccc(-c3nc(-c4ccc5c(c4)C(c4ccccc4)(c4ccccc4)c4ccccc4-5)nc(-c4cccc5c4sc4ccccc45)n3)cc2)cc1]

Further, a high polymer compound is usable for the electron transporting layer. For instance, poly[(9,9-dihexylfluorene-2,7-diyl)-co-(pyridine-3,5-diyl)](abbreviation: PF-Py), poly[(9,9-dioctylfluorene-2,7-diyl)-co-(2,2′-bipyridine-6,6′-diyl)](abbreviation: PF-BPy) and the like are usable.

Electron Injecting Layer

The electron injecting layer is a layer containing a highly electron-injectable substance. Examples of a material for the electron injecting layer include an alkali metal, alkaline earth metal and a compound thereof, examples of which include lithium (Li), cesium (Cs), calcium (Ca), lithium fluoride (LiF), cesium fluoride (CsF), calcium fluoride (CaF₂), and lithium oxide (LiOx). In addition, the alkali metal, alkaline earth metal or the compound thereof may be added to the substance exhibiting the electron transportability in use. Specifically, for instance, magnesium (Mg) added to Alq may be used. In this case, the electrons can be more efficiently injected from the cathode.

Alternatively, the electron injecting layer may be provided by a composite material in a form of a mixture of the organic compound and the electron donor. Such a composite material exhibits excellent electron injectability and electron transportability since electrons are generated in the organic compound by the electron donor. In this case, the organic compound is preferably a material excellent in transporting the generated electrons. Specifically, the above examples (e.g., the metal complex and the hetero aromatic compound) of the substance forming the electron transporting layer are usable. As the electron donor, any substance exhibiting electron donating property to the organic compound is usable. Specifically, the electron donor is preferably alkali metal, alkaline earth metal and rare earth metal such as lithium, cesium, magnesium, calcium, erbium and ytterbium. The electron donor is also preferably alkali metal oxide and alkaline earth metal oxide such as lithium oxide, calcium oxide, and barium oxide. Moreover, a Lewis base such as magnesium oxide is usable. Further, the organic compound such as tetrathiafulvalene (abbreviation: TTF) is usable.

Layer Formation Method

A method for forming each layer of the organic EL device in the exemplary embodiment is subject to no limitation except for the above particular description. However, known methods of dry film-forming such as vacuum deposition, sputtering, plasma or ion plating and wet film-forming such as spin coating, dipping, flow coating or ink-jet are applicable.

Film Thickness

A film thickness of each of the organic layers of the organic EL device in the exemplary embodiment is not limited unless otherwise specified in the above. In general, the thickness preferably ranges from several nanometers to 1 μm because excessively small film thickness is likely to cause defects (e.g. pin holes) and excessively large thickness leads to the necessity of applying high voltage and consequent reduction in efficiency.

Emission Wavelength of Organic EL Device

The organic electroluminescence device according to the exemplary embodiment preferably emits light having a main peak wavelength in a range from 430 nm to 480 nm when the organic electroluminescence device is driven.

The main peak wavelength of the light emitted from the organic EL device when being driven is measured as follows. Voltage is applied on the organic EL devices such that a current density becomes 10 mA/cm², where spectral radiance spectrum is measured by a spectroradiometer CS-2000 (manufactured by Konica Minolta, Inc.). A peak wavelength of an emission spectrum, at which the luminous intensity of the resultant spectral radiance spectrum is at the maximum, is measured and defined as the main peak wavelength (unit: nm).

The organic EL device according to the exemplary embodiment can improve device performance. Moreover, in the organic EL device according to the exemplary embodiment in which a plurality of emitting layers are layered, since the emitting layer close to the anode (first emitting layer) contains the compound according to the first exemplary embodiment, a balance between a luminous efficiency and a lifetime can be expected to be favorable.

### Fourth Exemplary Embodiment

Electronic Device

An electronic device according to a fourth exemplary embodiment is installed with any one of the organic EL devices according to the above exemplary embodiments. Examples of the electronic device include a display device and a light-emitting unit. Examples of the display device include a display component (e.g., an organic EL panel module), TV, mobile phone, tablet and personal computer. Examples of the light-emitting unit include an illuminator and a vehicle light.

### Modification of Exemplary Embodiments

The scope of the invention is not limited by the above-described exemplary embodiments but includes any modification and improvement as long as such modification and improvement are compatible with the invention.

For instance, the emitting layer does not necessarily include a single layer or two emitting layers, but may include a plurality, i.e., exceeding two, of emitting layers layered. In a case where the organic EL device includes a plurality of emitting layers, it is only required that one or two of the emitting layers satisfy the conditions described in the above exemplary embodiments. For instance, the rest of the emitting layers may be a fluorescent emitting layer or a phosphorescent emitting layer with use of emission caused by electron transfer from the triplet excited state directly to the ground state.

When the organic EL device includes a plurality of emitting layers, these emitting layers may be mutually adjacently provided, or may form a so-called tandem organic EL device, in which a plurality of emitting units are layered via an intermediate layer.

For instance, a blocking layer may be provided adjacent to at least one of a side of the emitting layer close to the anode or a side of the emitting layer close to the cathode. The blocking layer is preferably provided in contact with the emitting layer to block at least any of holes, electrons, excitons or combinations thereof.

For instance, when the blocking layer is provided in contact with the side of the emitting layer close to the cathode, the blocking layer permits transport of electrons, and blocks holes from reaching a layer provided closer to the cathode (e.g., the electron transporting layer) beyond the blocking layer. When the organic EL device includes the electron transporting layer, the blocking layer is preferably interposed between the emitting layer and the electron transporting layer.

When the blocking layer is provided in contact with the side of the emitting layer close to the anode, the blocking layer permits transport of holes and blocks electrons from reaching a layer provided closer to the anode (e.g., the hole transporting layer) beyond the blocking layer. When the organic EL device includes the hole transporting layer, the blocking layer is preferably interposed between the emitting layer and the hole transporting layer.

Alternatively, the blocking layer may be provided adjacent to the emitting layer so that the excitation energy does not leak out from the emitting layer toward neighboring layer(s). The blocking layer blocks excitons generated in the emitting layer from being transferred to a layer(s) (e.g., the electron transporting layer and the hole transporting layer) closer to the electrode(s) beyond the blocking layer.

The emitting layer is preferably bonded with the blocking layer.

Specific structure, shape and the like of the components in the invention may be designed in any manner as long as an object of the invention can be achieved.

### EXAMPLES

The invention will be described in further detail with reference to Examples. It should be noted that the scope of the invention is by no means limited to Examples.

Compounds Structures of a compound represented by a formula (1000B) used for manufacturing organic EL devices in Examples 1 to 31 are shown below.

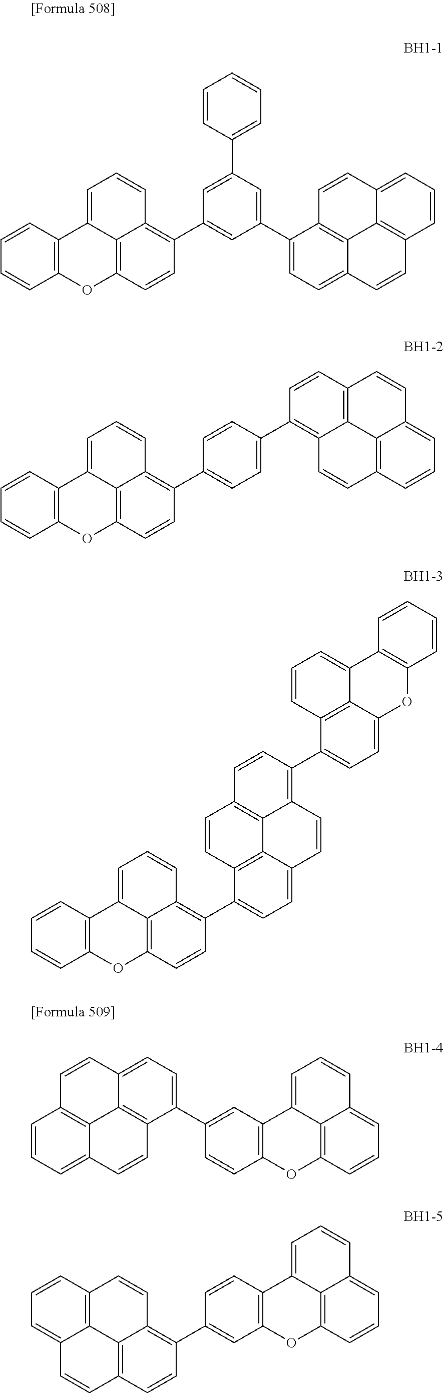 [SMILES: c1ccc(-c2cc(-c3ccc4c5c(cccc35)-c3ccccc3O4)cc(-c3ccc4ccc5cccc6ccc3c4c56)c2)cc1 ; c1ccc2c(c1)Oc1ccc(-c3ccc(-c4ccc5ccc6cccc7ccc4c5c67)cc3)c3cccc-2c13 ; c1ccc2c(c1)Oc1ccc(-c3ccc4ccc5c(-c6ccc7c8c(cccc68)-c6ccccc6O7)ccc6ccc3c4c65)c3cccc-2c13 ; c1cc2c3c(cccc3c1)-c1cc(-c3ccc4ccc5cccc6ccc3c4c56)ccc1O2 ; c1cc2c3c(cccc3c1)-c1ccc(-c3ccc4ccc5cccc6ccc3c4c56)cc1O2]

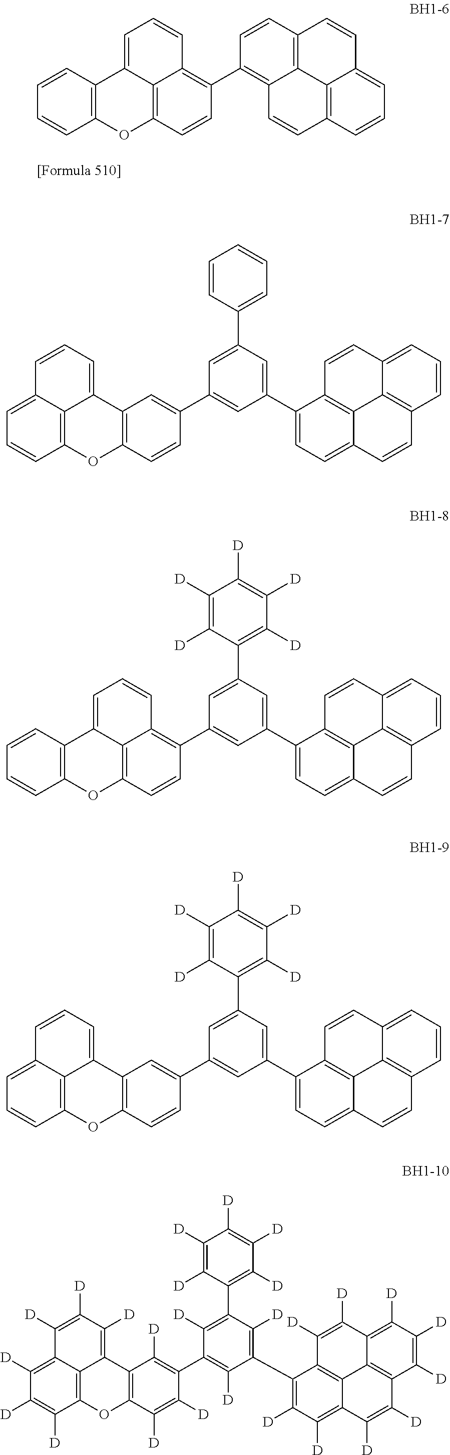

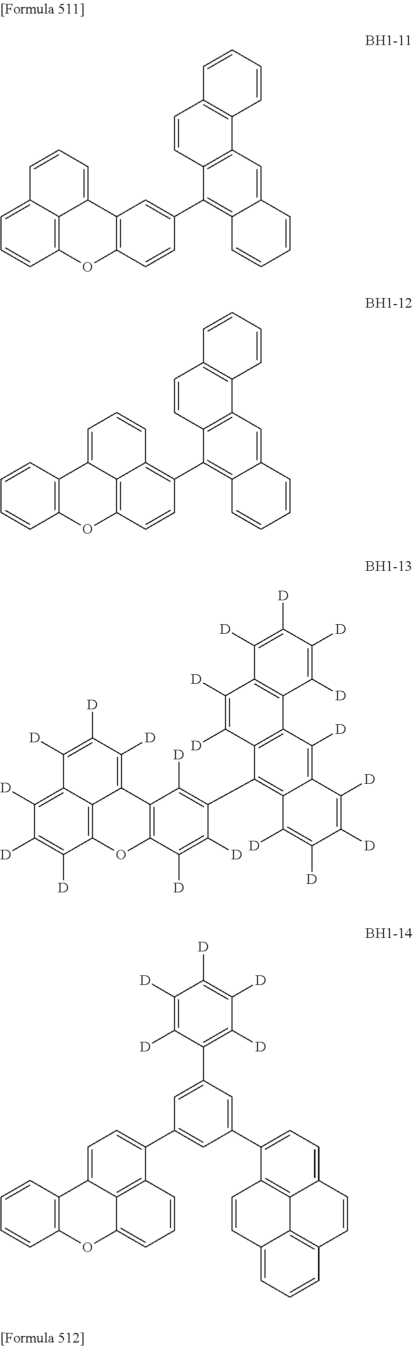 [SMILES: c1ccc2c(-c3ccc4c(c3)-c3cccc5cccc(c35)O4)c3ccc4ccccc4c3cc2c1 ; c1ccc2c(c1)Oc1ccc(-c3c4ccccc4cc4c3ccc3ccccc34)c3cccc-2c13 ; [2H]c1c([2H])c(-c2c3c([2H])c([2H])c([2H])c([2H])c3c([2H])c3c2c([2H])c([2H])c2c([2H])c([2H])c([2H])c([2H])c23)c([2H])c2c1Oc1c([2H])c([2H])c([2H])c3c([2H])c([2H])c([2H])c-2c13 ; [2H]c1c([2H])c([2H])c(-c2cc(-c3ccc4c5c(cccc35)Oc3ccccc3-4)cc(-c3ccc4ccc5cccc6ccc3c4c56)c2)c([2H])c1[2H]]

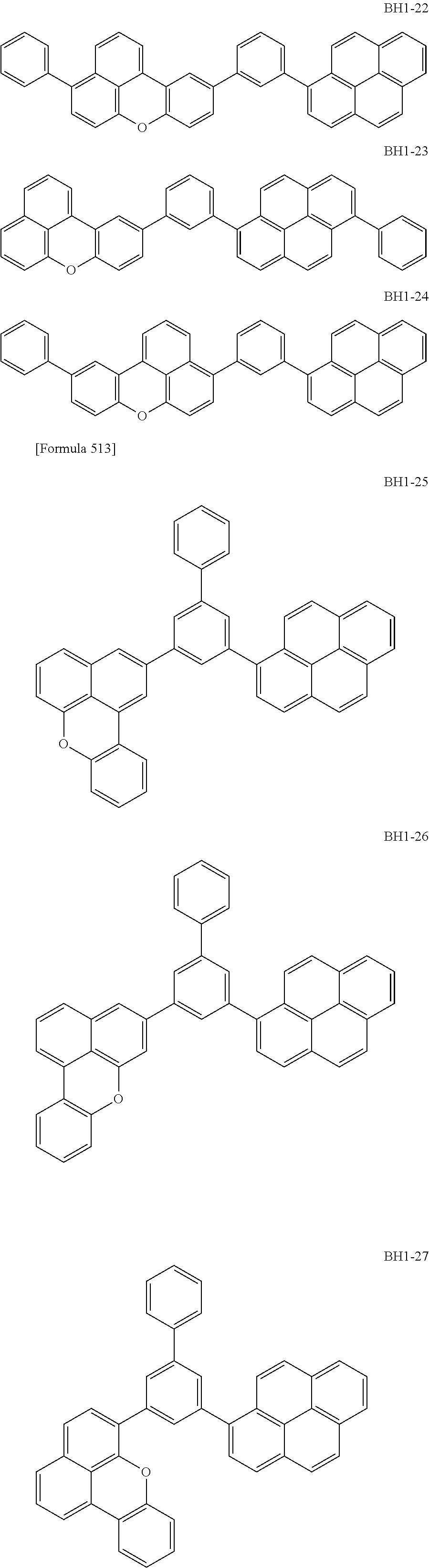 [SMILES: c1ccc(-c2ccc3c4c(cccc24)-c2cc(-c4cccc(-c5ccc6ccc7cccc8ccc5c6c78)c4)ccc2O3)cc1 ; c1ccc(-c2ccc3ccc4c(-c5cccc(-c6ccc7c(c6)-c6cccc8cccc(c68)O7)c5)ccc5ccc2c3c54)cc1 ; c1ccc(-c2ccc3c(c2)-c2cccc4c(-c5cccc(-c6ccc7ccc8cccc9ccc6c7c89)c5)ccc(c24)O3)cc1 ; c1ccc(-c2cc(-c3cc4c5c(cccc5c3)Oc3ccccc3-4)cc(-c3ccc4ccc5cccc6ccc3c4c56)c2)cc1 ; c1ccc(-c2cc(-c3cc4c5c(cccc5c3)-c3ccccc3O4)cc(-c3ccc4ccc5cccc6ccc3c4c56)c2)cc1 ; c1ccc(-c2cc(-c3ccc4cccc5c4c3Oc3ccccc3-5)cc(-c3ccc4ccc5cccc6ccc3c4c56)c2)cc1]

Structures of compounds used for manufacturing organic EL devices in Comparatives 4 to 7 are shown below.

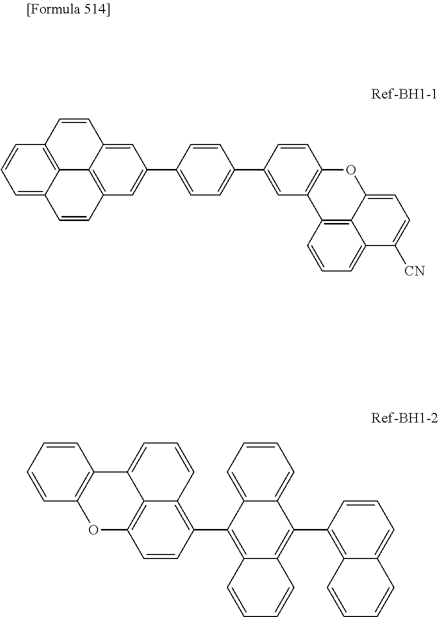 [SMILES: N#Cc1ccc2c3c(cccc13)-c1cc(-c3ccc(-c4cc5ccc6cccc7ccc(c4)c5c67)cc3)ccc1O2 ; c1ccc2c(c1)Oc1ccc(-c3c4ccccc4c(-c4cccc5ccccc45)c4ccccc34)c3cccc-2c13]

Structures of other compounds used for manufacturing organic EL devices in Examples 1 to 31 and Comparatives 1 to 9 are shown below.

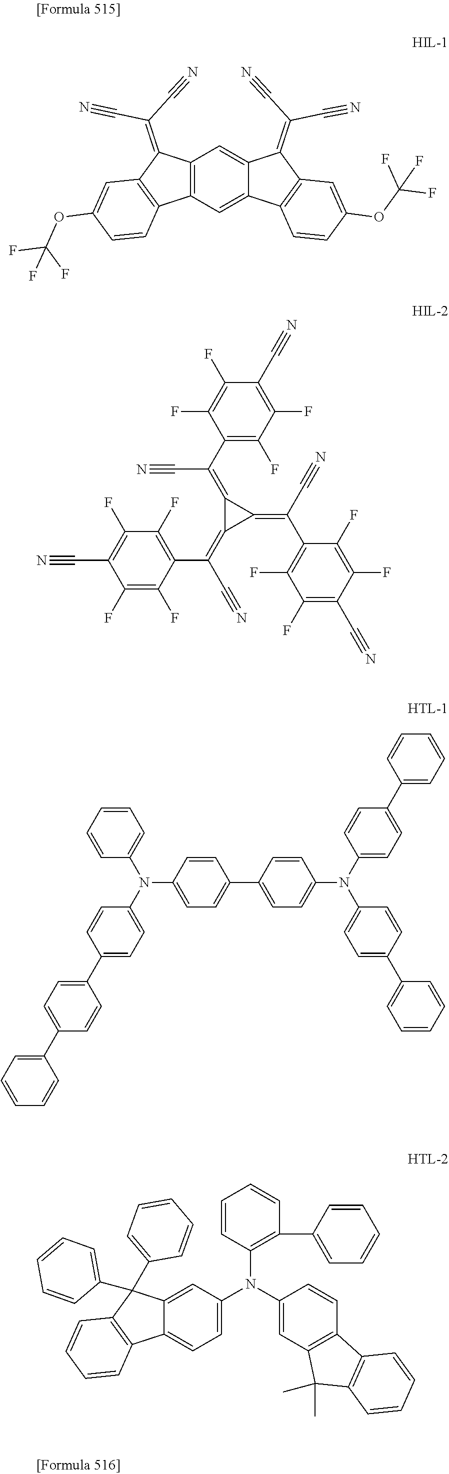 [SMILES: N#CC(C#N)=C1c2cc(OC(F)(F)F)ccc2-c2cc3c(cc21)C(=C(C#N)C#N)c1cc(OC(F)(F)F)ccc1-3 ; N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; CC1(C)c2ccccc2-c2ccc(N(c3ccc4c(c3)C(c3ccccc3)(c3ccccc3)c3ccccc3-4)c3ccccc3-c3ccccc3)cc21]

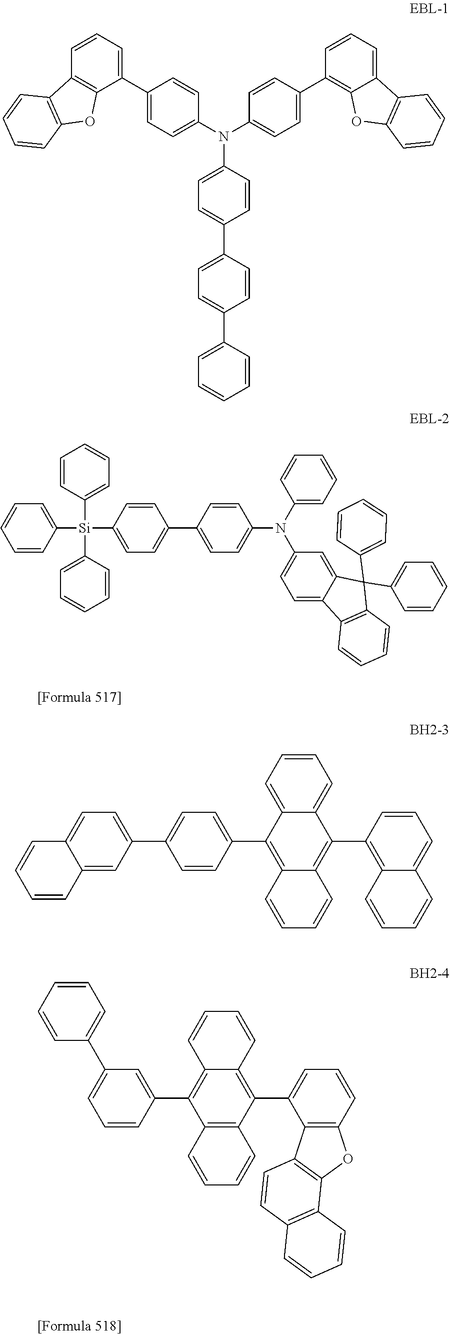

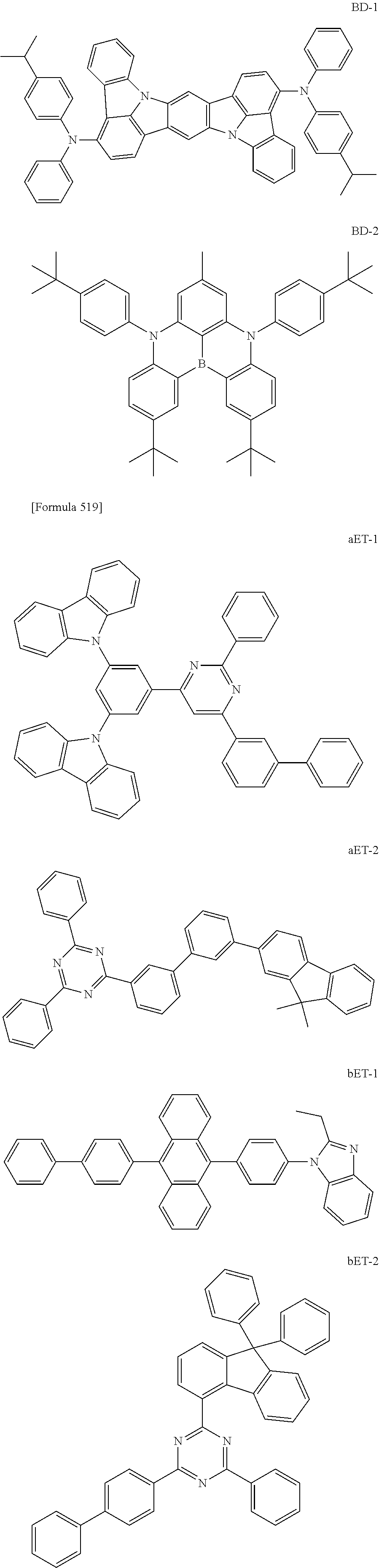

Preparation of Organic EL Device

The organic EL devices were manufactured and evaluated as follows.

### Example 1

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. A film thickness of the ITO transparent electrode was 130 nm.

The cleaned glass substrate having the transparent electrode line was attached to a substrate holder of a vacuum deposition apparatus. First, a compound HIL-1 was vapor-deposited on a surface of the glass substrate, where the transparent electrode line was provided, to cover the transparent electrode, thereby forming a 5-nm-thick hole injecting layer.

Subsequent to the formation of the hole injecting layer, a compound HTL-1 was vapor-deposited to form an 80-nm-thick first hole transporting layer.

Subsequent to the formation of the first hole transporting layer, a compound EBL-1 was vapor-deposited to form a 10-nm-thick second hole transporting layer (also referred to as an electron blocking layer).

A compound BH1-1 as the first compound and a compound BD-1 as the third compound were co-deposited on the second hole transporting layer such that a ratio of the compound BD-1 accounted for 2 mass %, thereby forming a 5-nm-thick first emitting layer.

A compound BH2-3 as the second compound and a compound BD-1 as the fourth compound were co-deposited on the first emitting layer such that a ratio of the compound BD-1 accounted for 2 mass %, thereby forming a 20-nm-thick second emitting layer.

A compound aET-1 was vapor-deposited on the second emitting layer to form a 10-nm-thick first electron transporting layer (also referred to as a hole blocking layer).

A compound bET-1 was vapor-deposited on the first electron transporting layer to form a 15-nm-thick second electron transporting layer.

LiF was vapor-deposited on the second electron transporting layer to form a 1-nm-thick electron injecting layer.

Metal (Al) was vapor-deposited on the electron injecting layer to form an 80-nm-thick cathode.

A device arrangement of the organic EL device in Example 1 is roughly shown as follows.
ITO(130)/HIL-1(5)/HTL-1(80)/EBL-1(10)/BH1-1:BD-1 (5.98%:2%)/BH2-3:BD-1 (20.98%:2%)/aET-1(10)/bET-1(15)/LiF(1)/Al(80)

The numerals in parentheses represent film thickness (unit: nm).

The numerals (98%:2%) represented by percentage in the same parentheses indicate a ratio (mass %) between the compound BH1-1 or BH2-3 and the compound BD-1 in the first emitting layer or the second emitting layer. Similar notations apply to the description below.

### Examples 2, 3, 4, 5, 6, 7, 8, 9, 10, 11, 12, 13, and 14

Each of organic EL devices of Examples 2, 3, 4, 5, 6, 7, 8, 9, 10, 11, 12, 13, and 14 was manufactured in the same manner as in Example 1 except that the first compound of the first emitting layer was replaced by compounds shown in Table 1.

### Comparative 1

An organic EL device of Comparative 1 was manufactured in the same manner as in Example 1 except that a 25-nm-thick second emitting layer was formed on the second hole transporting layer without forming the first emitting layer as shown in Table 1.

### Comparative 2

An organic EL device of Comparative 2 was manufactured in the same manner as in Example 1 except that a 25-nm-thick first emitting layer was formed as the emitting layer and the first electron transporting layer was formed on the first emitting layer without forming the second emitting layer as shown in Table 1.

### Comparatives 3, 4, and 5

Each of organic EL devices of Comparatives 3, 4, and 5 was manufactured in the same manner as in Comparative 2 except that the first compound of the first emitting layer was replaced by compounds shown in Table 1.

### Comparatives 6 and 7

Each of organic EL devices of Comparatives 6 and 7 was manufactured in the same manner as in Example 1 except that the first compound of the first emitting layer was replaced by compounds shown in Table 1.

TABLE 1

| | First Emitting Layer | | | Second Emitting Layer | | | | |
|---|---|---|---|---|---|---|---|---|
| | First | Third | Thickness | Second | Fourth | Thickness | EQE | LT95 |
| | Compound | Compound | [nm] | Compound | Compound | [nm] | [%] | [h] |
| Ex. 1 | BH1-1 | BD-1 | 5 | BH2-3 | BD-1 | 20 | 10.3 | 140 |
| Ex. 2 | BH1-2 | BD-1 | 5 | BH2-3 | BD-1 | 20 | 10.1 | 135 |
| Ex. 3 | BH1-3 | BD-1 | 5 | BH2-3 | BD-1 | 20 | 9.9 | 130 |
| Ex. 4 | BH1-4 | BD-1 | 5 | BH2-3 | BD-1 | 20 | 10.2 | 140 |
| Ex. 5 | BH1-5 | BD-1 | 5 | BH2-3 | BD-1 | 20 | 10.0 | 135 |
| Ex. 6 | BH1-6 | BD-1 | 5 | BH2-3 | BD-1 | 20 | 10.0 | 135 |
| Ex. 7 | BH1-7 | BD-1 | 5 | BH2-3 | BD-1 | 20 | 10.1 | 165 |
| Ex. 8 | BH1-8 | BD-1 | 5 | BH2-3 | BD-1 | 20 | 10.3 | 160 |
| Ex. 9 | BH1-9 | BD-1 | 5 | BH2-3 | BD-1 | 20 | 10.1 | 195 |
| Ex. 10 | BH1-10 | BD-1 | 5 | BH2-3 | BD-1 | 20 | 10.0 | 215 |
| Ex. 11 | BH1-11 | BD-1 | 5 | BH2-3 | BD-1 | 20 | 10.5 | 125 |
| Ex. 12 | BH1-12 | BD-1 | 5 | BH2-3 | BD-1 | 20 | 10.5 | 125 |
| Ex. 13 | BH1-13 | BD-1 | 5 | BH2-3 | BD-1 | 20 | 10.5 | 150 |
| Ex. 14 | BH1-14 | BD-1 | 5 | BH2-3 | BD-1 | 20 | 10.4 | 150 |
| Comp. 1 | — | — | — | BH2-3 | BD-1 | 25 | 9.8 | 130 |
| Comp. 2 | BH1-1 | BD-1 | 25 | — | — | — | 8.2 | 10 |
| Comp. 3 | BH1-3 | BD-1 | 25 | — | — | — | 7.9 | 8 |
| Comp. 4 | Ref-BH1-1 | BD-1 | 25 | — | — | — | 7.1 | 4 |
| Comp. 5 | Ref-BH1-2 | BD-1 | 25 | — | — | — | 8.1 | 55 |
| Comp. 6 | Ref-BH1-1 | BD-1 | 5 | BH2-3 | BD-1 | 20 | 7.4 | 25 |
| Comp. 7 | Ref-BH1-2 | BD-1 | 5 | BH2-3 | BD-1 | 20 | 8.2 | 75 |

Evaluation of Organic EL Device

The organic EL devices in Examples 1 to 31 and Comparatives 1 to 9 were evaluated as follows. Evaluation results are shown in Tables 1, 2, 3, and 4.

External Quantum Efficiency (EQE)

Voltage was applied on the organic EL devices so that a current density was 10 mA/cm², where spectral radiance spectrum was measured by a spectroradiometer 08-2000 (manufactured by Konica Minolta, Inc.). The external quantum efficiency EQE (unit: %) was calculated based on the obtained spectral-radiance spectra, assuming that the spectra was provided under a Lambertian radiation.

Lifetime (LT95)

Voltage was applied on the resultant organic EL devices such that a current density was 50 mA/cm², where a time (LT95 (unit: hr)) elapsed before a luminance intensity was reduced to 95% of the initial luminance intensity was measured.

### Example 15

An organic EL device of Example 15 was manufactured in the same manner as in Example 1 except that the third compound of the first emitting layer and the fourth compound of the second emitting layer were replaced by compounds shown in Table 2.

### Examples 16, 17, 18, 19, 20, 21, 22, 23, and 24

Each of organic EL devices of Examples 16, 17, 18, 19, 20, 21, 22, 23, and 24 was manufactured in the same manner as in Example 15 except that the first compound of the first emitting layer was replaced by compounds shown in Table 2.

### Comparative 8

An organic EL device of Comparative 8 was manufactured in the same manner as in Example 15 except that a 25-nm-thick second emitting layer was formed on the second hole transporting layer without forming the first emitting layer as shown in Table 2.

TABLE 2

| | First Emitting Layer | | | Second Emitting Layer | | | | |
|---|---|---|---|---|---|---|---|---|
| | First | Third | Thickness | Second | Fourth | Thickness | EQE | LT95 |
| | Compound | Compound | [nm] | Compound | Compound | [nm] | [%] | [h] |
| Ex. 15 | BH1-1 | BD-2 | 5 | BH2-3 | BD-2 | 20 | 9.5 | 140 |
| Ex. 16 | BH1-6 | BD-2 | 5 | BH2-3 | BD-2 | 20 | 9.4 | 135 |
| Ex. 17 | BH1-7 | BD-2 | 5 | BH2-3 | BD-2 | 20 | 9.3 | 165 |
| Ex. 18 | BH1-8 | BD-2 | 5 | BH2-3 | BD-2 | 20 | 9.5 | 160 |
| Ex. 19 | BH1-9 | BD-2 | 5 | BH2-3 | BD-2 | 20 | 9.3 | 190 |
| Ex. 20 | BH1-10 | BD-2 | 5 | BH2-3 | BD-2 | 20 | 9.2 | 210 |
| Ex. 21 | BH1-11 | BD-2 | 5 | BH2-3 | BD-2 | 20 | 9.7 | 120 |
| Ex. 22 | BH1-12 | BD-2 | 5 | BH2-3 | BD-2 | 20 | 9.7 | 115 |
| Ex. 23 | BH1-13 | BD-2 | 5 | BH2-3 | BD-2 | 20 | 9.7 | 145 |
| Ex. 24 | BH1-14 | BD-2 | 5 | BH2-3 | BD-2 | 20 | 9.6 | 150 |
| Comp. 8 | — | — | — | BH2-3 | BD-2 | 25 | 9.2 | 130 |

### Example 25

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. A film thickness of the ITO transparent electrode was 130 nm.

The cleaned glass substrate having the transparent electrode line was attached to a substrate holder of a vacuum deposition apparatus. First, a compound HTL-2 and a compound HIL-2 were co-deposited on a surface of the glass substrate, where the transparent electrode line was provided, to cover the transparent electrode, thereby forming a 10-nm-thick hole injecting layer. The compound HTL-2 and the compound HIL-2 were set at 90 mass % and 10 mass %, respectively, in ratio in the hole injecting layer.

Subsequent to the formation of the hole injecting layer, the compound HTL-2 was vapor-deposited to form an 85-nm-thick first hole transporting layer.

Subsequent to the formation of the first hole transporting layer, a compound EBL-2 was vapor-deposited to form a 5-nm-thick second hole transporting layer (also referred to as an electron blocking layer).

The compound BH1-1 as the first compound and a compound BD-2 as the third compound were co-deposited on the second hole transporting layer such that a ratio of the compound BD-2 accounted for 2 mass %, thereby forming a 5-nm-thick first emitting layer.

A compound BH2-4 as the second compound and the compound BD-2 as the fourth compound were co-deposited on the first emitting layer such that a ratio of the compound BD-2 accounted for 2 mass %, thereby forming a 15-nm-thick second emitting layer.

A compound aET-2 was vapor-deposited on the second emitting layer to form a 5-nm-thick first electron transporting layer (also referred to as a hole blocking layer).

A compound bET-2 and a compound Liq were co-deposited on the first electron transporting layer to form a 25-nm-thick second electron transporting layer. The compound bET-2 and the compound Liq were set at 50 mass % and 50 mass %, respectively, in ratio in the second electron transporting layer. Liq is an abbreviation of (8-quinolinolato)lithium ((8-Quinolinolato)lithium).

The compound Liq was vapor-deposited on the second electron transporting layer to form a 1-nm-thick electron injecting layer.

Metal (Al) was vapor-deposited on the electron injecting layer to form an 80-nm-thick cathode.

A device arrangement of the organic EL device of Example 25 is roughly shown as follows.
ITO(130)/HTL-2:HIL-2 (10.90%:10%)/HTL-2(85)/EBL-2(5)/BH1-1:BD-2 (5.98%:2%)/BH2-4:BD-2 (15.98%:2%)/aET-2(5)/bET-2:Liq(25.50%:50%)/Liq(1)/Al(80)

The numerals in parentheses represent film thickness (unit: nm).

The numerals (90%:10%) represented by percentage in the same parentheses indicate a ratio (mass %) between the compound HTL-2 and the compound HIL-2 in the hole injecting layer. The numerals (98%:2%) represented by percentage in the same parentheses indicate a ratio (mass %) between the compound BH1-1 or BH2-4 and the compound BD-2 in the first emitting layer or the second emitting layer. The numerals (50%:50%) represented by percentage in the same parentheses indicate a ratio (mass %) between the compound bET-2 and the compound Liq in the second electron transporting layer. Similar notations apply to the description below.

### Comparative 9

An organic EL device of Comparative 9 was manufactured in the same manner as in Example 25 except that a 20-nm-thick second emitting layer was formed on the second hole transporting layer without forming the first emitting layer as shown in Table 3.

TABLE 3

| | First Emitting Layer | | | Second Emitting Layer | | | | |
|---|---|---|---|---|---|---|---|---|
| | First | Third | Thickness | Second | Fourth | Thickness | EQE | LT95 |
| | Compound | Compound | [nm] | Compound | Compound | [nm] | [%] | [h] |
| Ex. 25 | BH1-1 | BD-2 | 5 | BH2-4 | BD-2 | 15 | 9.9 | 80 |
| Comp. 9 | — | — | — | BH2-4 | BD-2 | 20 | 9.9 | 40 |

### Example 26

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. A film thickness of the ITO transparent electrode was 130 nm.

The cleaned glass substrate having the transparent electrode line was attached to a substrate holder of a vacuum deposition apparatus. First, the compound HIL-1 was vapor-deposited on a surface of the glass substrate, where the transparent electrode line was provided, to cover the transparent electrode, thereby forming a 5-nm-thick hole injecting layer.

Subsequent to the formation of the hole injecting layer, the compound HTL-1 was vapor-deposited to form an 80-nm-thick first hole transporting layer.

Subsequent to the formation of the first hole transporting layer, the compound EBL-1 was vapor-deposited to form a 10-nm-thick second hole transporting layer (also referred to as the electron blocking layer).

A compound BH1-22 as the first compound and the compound BD-1 as the third compound were co-deposited on the second hole transporting layer such that a ratio of the compound BD-1 accounted for 2 mass %, thereby forming a 5-nm-thick first emitting layer.

A compound BH2-3 as the second compound and the compound BD-1 as the fourth compound were co-deposited on the first emitting layer such that a ratio of the compound BD-1 accounted for 2 mass %, thereby forming a 20-nm-thick second emitting layer.

A compound aET-1 was vapor-deposited on the second emitting layer to form a 10-nm-thick first electron transporting layer (also referred to as the hole blocking layer).

The compound bET-1 was vapor-deposited on the first electron transporting layer to form a 15-nm-thick second electron transporting layer.

LiF was vapor-deposited on the second electron transporting layer to form a 1-nm-thick electron injecting layer.

Metal (Al) was vapor-deposited on the electron injecting layer to form an 80-nm-thick cathode.

A device arrangement of the organic EL device of Example 26 is roughly shown as follows.
ITO(130)/HIL-1(5)/HTL-1(80)/EBL-1(10)/BH1-22:BD-1 (5.98%:2%)/BH2-3:BD-1 (20.98%:2%)/aET-1(10)/bET-1(15)/LiF(1)/Al(80)

The numerals in parentheses represent film thickness (unit: nm).

The numerals (98%:2%) represented by percentage in the same parentheses indicate a ratio (mass %) between the compound BH1-22 or BH2-3 and the compound BD-1 in the first emitting layer or the second emitting layer. Similar notations apply to the description below.

### Examples 27, 28, 29, 30, and 31

Each of organic EL devices of Examples 27, 28, 29, 30, and 31 was manufactured in the same manner as in Example 26 except that the first compound of the first emitting layer was replaced by compounds shown in Table 4.

TABLE 4

| | First Emitting Layer | | | Second Emitting Layer | | | | |
|---|---|---|---|---|---|---|---|---|
| | First | Third | Thickness | Second | Fourth | Thickness | EQE | LT95 |
| | Compound | Compound | [nm] | Compound | Compound | [nm] | [%] | [h] |
| Ex. 26 | BH1-22 | BD-1 | 5 | BH2-3 | BD-1 | 20 | 10.1 | 193 |
| Ex. 27 | BH1-23 | BD-1 | 5 | BH2-3 | BD-1 | 20 | 10.3 | 143 |
| Ex. 28 | BH1-24 | BD-1 | 5 | BH2-3 | BD-1 | 20 | 10.3 | 171 |
| Ex. 29 | BH1-25 | BD-1 | 5 | BH2-3 | BD-1 | 20 | 10.3 | 130 |
| Ex. 30 | BH1-26 | BD-1 | 5 | BH2-3 | BD-1 | 20 | 10.1 | 95 |
| Ex. 31 | BH1-27 | BD-1 | 5 | BH2-3 | BD-1 | 20 | 9.9 | 132 |

### Synthesis of Compounds

### Synthesis Example 1: Synthesis of Compound BH1-1

The compound BH1-1 was synthesized through a synthesis pathway below.

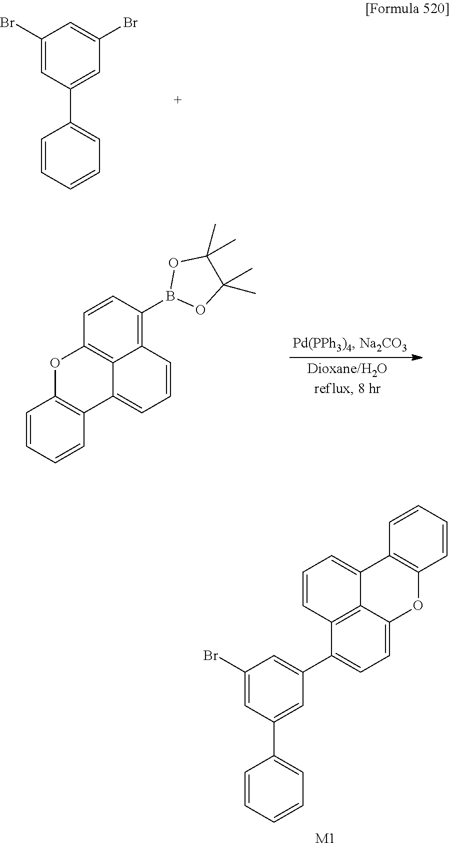

### Synthesis of Intermediate M1

Under argon atmosphere, 8.21 g of 3,5-dibromo-1,1′-biphenyl, 5.52 g of 4-(4,4,5,5-tetramethyl-1,3,2-dioxaborane-2-yl)benzo[kl]xanthene synthesized by a known method, 0.82 g of tetrakis(triphenylphosphine)palladium(0), 4.22 g of sodium carbonate, 150 mL of 1,4-dioxane, and 40 mL of water were put into a three-necked flask and refluxed for eight hours with stirring. After the reflux with stirring, the obtained solution was cooled to the room temperature. After the solution was cooled, the deposited solid was filtered and collected. The obtained solid was sequentially washed with water and methanol, and then repeatedly recrystallized with toluene to obtain 4.75 g of a light yellow solid (yield of 66%). The obtained compound was subjected to FD-MS (Field Desorption Mass Spectrometry) analysis and was identified as an intermediate M1.

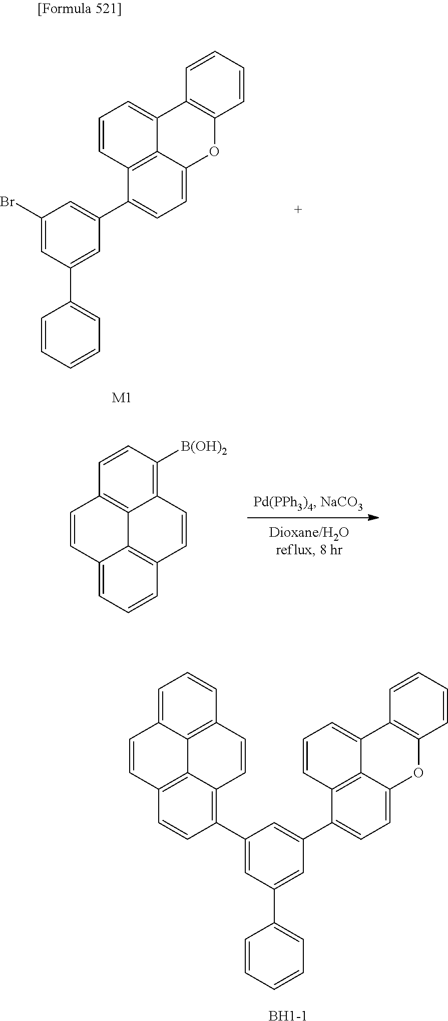

### Synthesis of Compound BH1-1

Under argon atmosphere, 13.21 g of the intermediate M1, 2.53 g of pyrene-1-ylboronic acid synthesized by a known method, 0.22 g of tetrakis(triphenylphosphine)palladium(0), 2.22 g of sodium carbonate, 50 mL of 1,4-dioxane, and 10 mL of water were put into a three-necked flask and refluxed for eight hours with stirring. After the reflux with stirring, the obtained solution was cooled to the room temperature. After the solution was cooled, the deposited solid was filtered and collected. The obtained solid was sequentially washed with water and methanol, and then repeatedly recrystallized with toluene to obtain 4.70 g of a light yellow solid (yield of 80%). As a result of FD-MS analysis, the light yellow solid was a compound BH1-1 and m/e was equal to 571 while a molecular weight was 570.69.

### Synthesis Example 2: Synthesis of Compound BH1-2

A compound BH1-2 was synthesized through a synthesis pathway below.

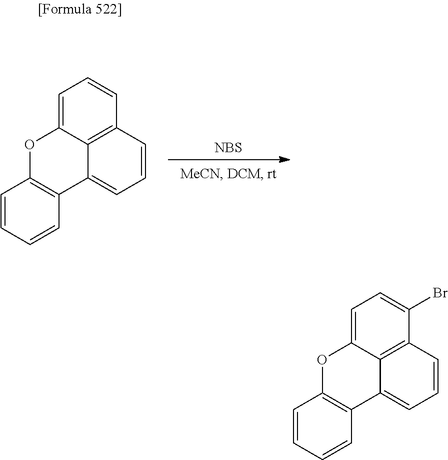

### Synthesis of 4-Bromobenzo[kl]xanthene

Under argon atmosphere, 13.5 g of N-bromosuccinimide was added to 400 mL of a mixed solution including 15.0 g of benzo[kl]xanthene in dichloromethane and acetonitrile (3:1). The obtained solution was stirred at the room temperature for ten minutes. After the solution was stirred, the deposited solid was filtered and collected. The obtained solid was washed with a mixed solvent of acetonitrile and methanol to obtain 15.6 g of a white solid (yield of 77%). As a result of FD-MS analysis, the white solid was a 4-bromobenzo[kl]xanthene compound and m/e was equal to 298 while a molecular weight was 297.15. DCM is the abbreviation for dichloromethane, MeCN is the abbreviation for acetonitrile, and NBS is the abbreviation for N-bromosuccinimide.

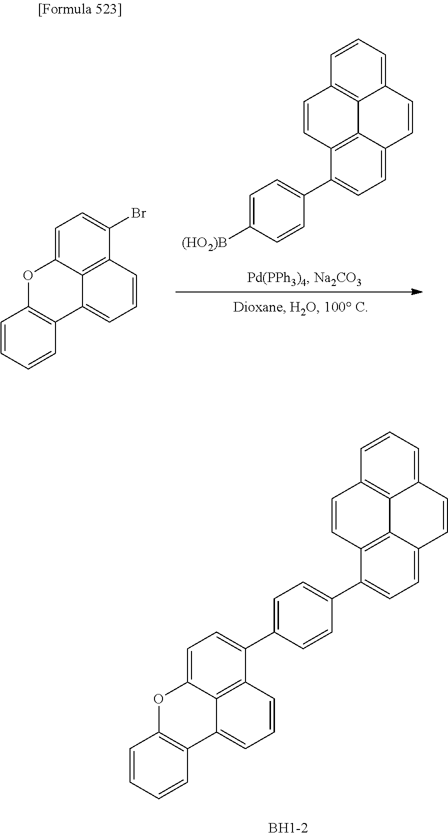

### Synthesis of Compound BH1-2

Under argon atmosphere, 3.00 g of 4-bromobenzo[kl]xanthene, 3.25 g of 4-(pyrene-1-yl)phenylboronic acid, 0.46 g of tetrakis(triphenylphosphine)palladium(0), 3.21 g of sodium carbonate, 50 mL of 1,4-dioxane, and 15 mL of ion-exchange water were put into a flask and refluxed for 18 hours with stirring. After the reflux with stirring, the obtained solution was cooled to the room temperature. After the solution was cooled, the deposited solid was filtered and collected. The obtained solid was sequentially washed with water and acetone, and then recrystallized with a mixed solvent of toluene and cyclohexane to obtain 2.34 of a light yellow solid (yield of 47%). As a result of FD-MS analysis, the light yellow solid was a compound BH1-2 and m/e was equal to 495 while a molecular weight was 494.59.

### Synthesis Example 3: Synthesis of Compound BH1-3

A compound BH1-3 was synthesized through a synthesis pathway below.

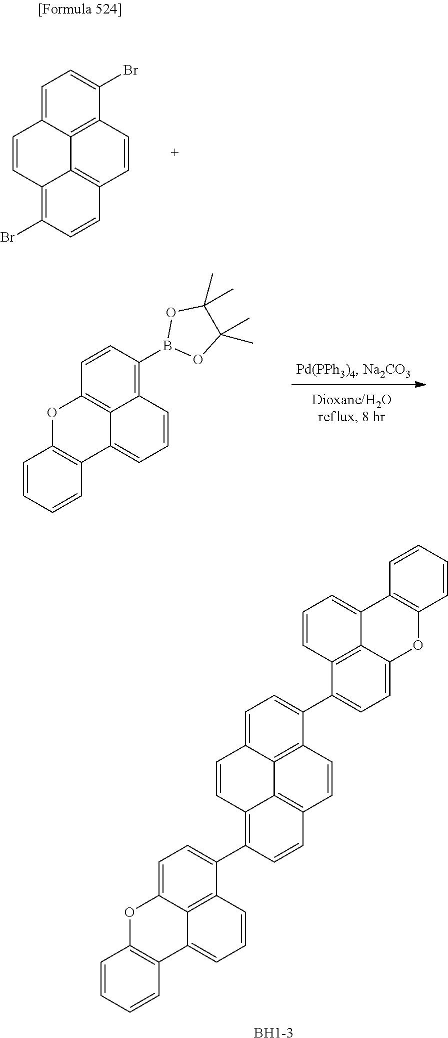 [SMILES: Brc1ccc2ccc3c(Br)ccc4ccc1c2c43 ; CC1(C)OB(c2ccc3c4c(cccc24)-c2ccccc2O3)OC1(C)C ; c1ccc2c(c1)Oc1ccc(-c3ccc4ccc5c(-c6ccc7c8c(cccc68)-c6ccccc6O7)ccc6ccc3c4c65)c3cccc-2c13 ; "BH1-3"]

### Synthesis of Compound BH1-3

Under argon atmosphere, 5.00 g of 1,6-dibromo pyrene, 9.59 g of 4-(4,4,5,5-tetramethyl-1,3,2-dioxaborane-2-yl)benzo[kl]xanthene synthesized by a known method, 0.32 g of tetrakis(triphenylphosphine)palladium(0), 2.88 g of sodium carbonate, 60 mL of 1,4-dioxane, and 9.9 mL of water were put into a three-necked flask and refluxed for eight hours with stirring. After the reflux with stirring, the obtained solution was cooled to the room temperature. After the solution was cooled, the deposited solid was filtered and collected. The obtained solid was sequentially washed with water and methanol, and then repeatedly recrystallized with toluene to obtain 6.59 g of a light yellow solid (yield of 75%). The obtained compound was subjected to FD-MS analysis and was identified as the compound BH1-3.

### Synthesis Example 4: Synthesis of Compound BH1-4

A compound BH1-4 was synthesized through a synthesis pathway below.

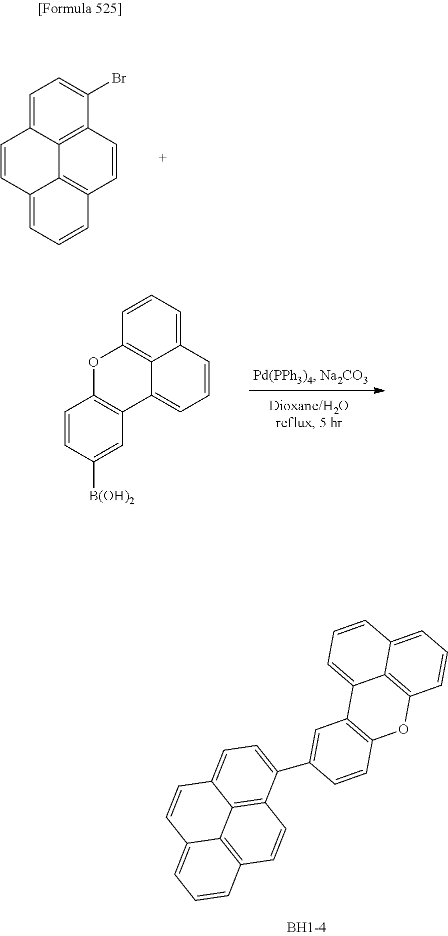 [SMILES: Brc1ccc2ccc3cccc4ccc1c2c34 ; OB(O)c1ccc2c(c1)-c1cccc3cccc(c13)O2 ; c1cc2c3c(cccc3c1)-c1cc(-c3ccc4ccc5cccc6ccc3c4c56)ccc1O2]

### Synthesis of Compound BH1-4

Under argon atmosphere, 4.00 g of 1-bromopyrene, 3.70 g of benzo[kl]xanthene-10-yl-boronic acid synthesized by a known method, 0.33 g of tetrakis(triphenylphosphine)palladium(0), 3.00 g of sodium carbonate, 61 mL of 1,4-dioxane, and 10.0 mL of water were put into a three-necked flask and refluxed for five hours with stirring. After the reflux with stirring, the obtained solution was cooled to the room temperature. After the solution was cooled, the deposited solid was filtered and collected. The obtained solid was sequentially washed with water and methanol, and then repeatedly recrystallized with toluene to obtain 3.50 g of a light yellow solid (yield of 59%). The obtained compound was subjected to FD-MS analysis and was identified as the compound BH1-4.

### Synthesis Example 5: Synthesis of Compound BH1-5

A compound BH1-5 was synthesized through a synthesis pathway below.

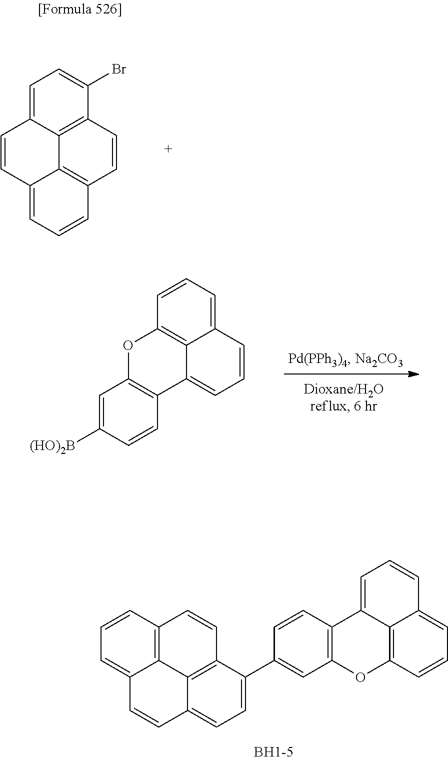 [SMILES: Brc1ccc2ccc3cccc4ccc1c2c34 ; OB(O)c1ccc2c(c1)Oc1cccc3cccc-2c13 ; c1cc2c3c(cccc3c1)-c1ccc(-c3ccc4ccc5cccc6ccc3c4c56)cc1O2]

### Synthesis of Compound BH1-5

Under argon atmosphere, 4.00 g of 1-bromopyrene, 3.83 g of benzo[kl]xanthene-9-yl-boronic acid synthesized by a known method, 0.65 g of tetrakis(triphenylphosphine)palladium(0), 2.42 g of sodium carbonate, 120 mL of 1,4-dioxane, and 10.0 mL of water were put into a three-necked flask and refluxed for six hours with stirring. After the reflux with stirring, the obtained solution was cooled to the room temperature. After the solution was cooled, the deposited solid was filtered and collected. The obtained solid was sequentially washed with water and methanol, and then repeatedly recrystallized with toluene to obtain 3.80 g of a light yellow solid (yield of 64%). The obtained compound was subjected to FD-MS analysis and was identified as the compound BH1-5.

### Synthesis Example 6: Synthesis of Compound BH1-6

A compound BH1-6 was synthesized through a synthesis pathway below.

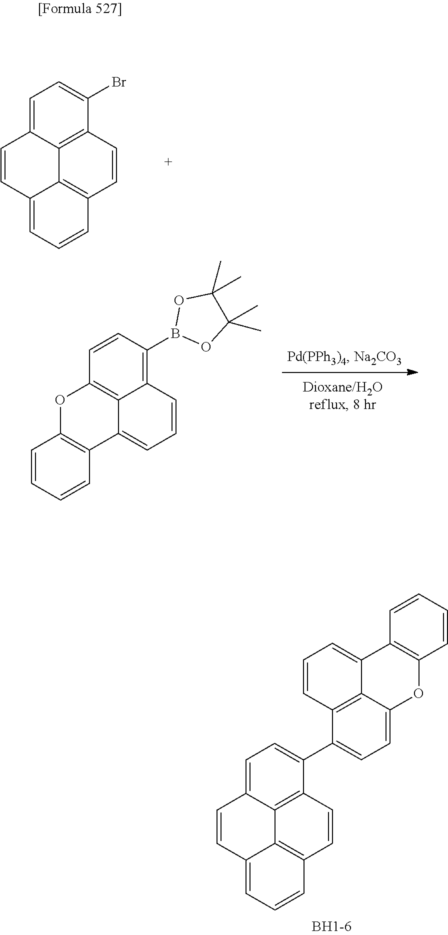 [SMILES: Brc1ccc2ccc3cccc4ccc1c2c34 ; CC1(C)OB(c2ccc3c4c(cccc24)-c2ccccc2O3)OC1(C)C ; c1ccc2c(c1)Oc1ccc(-c3ccc4ccc5cccc6ccc3c4c56)c3cccc-2c13]

### Synthesis of Compound BH1-6

Under argon atmosphere, 3.01 g of 1-bromopyrene, 3.70 g of 4-(4,4,5,5-tetramethyl-1,3,2-dioxaborane-2-yl)-benzo[kl]xanthene synthesized by a known method, 0.50 g of tetrakis(triphenylphosphine)palladium(0), 1.79 g of sodium carbonate, 100 mL of 1,4-dioxane, and 10 mL of water were put into a three-necked flask and refluxed for eight hours with stirring. After the reflux with stirring, the obtained solution was cooled to the room temperature. After the solution was cooled, the deposited solid was filtered and collected. The obtained solid was sequentially washed with water and methanol, and then repeatedly recrystallized with toluene to obtain 3.09 g of a light yellow solid (yield of 69%). The obtained compound was subjected to FD-MS analysis and was identified as the compound BH1-6.

### Synthesis Example 7: Synthesis of Compound BH1-7

An intermediate M2 was synthesized through a synthesis pathway below.

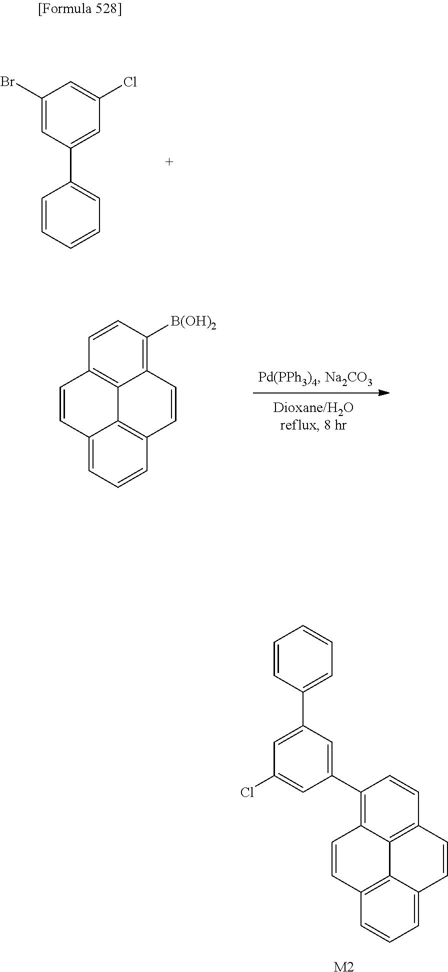

### Synthesis of Intermediate M2

Under argon atmosphere, 5.00 g of 3-chloro-5-bromo-1,1′-biphenyl, 4.60 g of pyrene-1-ylboronic acid, 0.648 g of tetrakis(triphenylphosphine)palladium(0), 3.96 g of sodium carbonate, 20 mL of 1,4-dioxane, and 20 mL of water were put into a three-necked flask and refluxed for eight hours with stirring. After the reflux with stirring, the obtained solution was cooled to the room temperature. After the solution was cooled, the deposited solid was filtered and collected. The obtained solid was sequentially washed with water and methanol, and then repeatedly recrystallized with toluene to obtain 7.16 g of a light yellow solid (yield of 99%). The obtained compound was subjected to FD-MS (Field Desorption Mass Spectrometry) analysis and was identified as the intermediate M2.

A compound BH1-7 was synthesized through a synthesis pathway below.

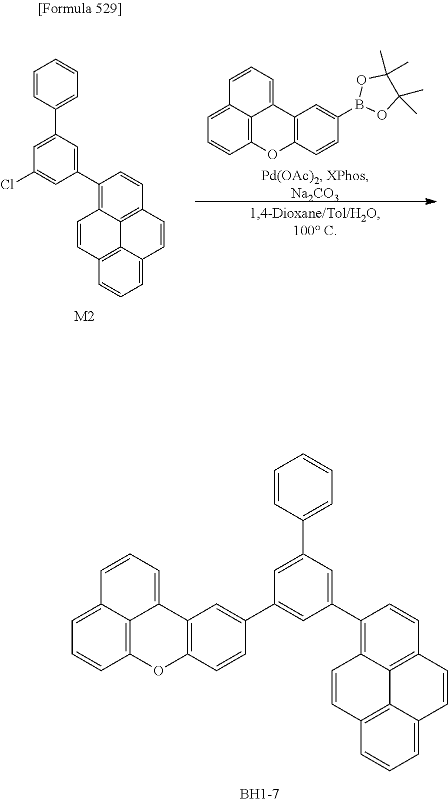 [SMILES: Clc1cc(-c2ccccc2)cc(-c2ccc3ccc4cccc5ccc2c3c45)c1 ; CC1(C)OB(c2ccc3c(c2)-c2cccc4cccc(c24)O3)OC1(C)C ; "M2" ; c1ccc(-c2cc(-c3ccc4c(c3)-c3cccc5cccc(c35)O4)cc(-c3ccc4ccc5cccc6ccc3c4c56)c2)cc1]

### Synthesis of Compound BH1-7

Under argon atmosphere, 60.0 g of the intermediate M2 (chloro intermediate), 55.8 g of a boronic acid pinacol intermediate synthesized by a known method, 1.05 g of palladium acetate(II), 32.7 g of sodium carbonate, 500 mL of toluene, 450 mL of 1,4-dioxane, and 200 mL of ion-exchange water were put into a flask and refluxed for six hours with stirring. After the reflux with stirring, the obtained solution was cooled to the room temperature. After the solution was cooled, the deposited solid was filtered and collected. The obtained solid was sequentially washed with water and acetone, and then recrystallized with a mixed solvent of toluene and hexane to obtain 64.3 g of a light yellow solid (yield of 72%). As a result of FD-MS analysis, the light yellow solid was the compound BH1-7 and m/e was equal to 571 while a molecular weight was 570.69.

### Synthesis Example 8: Synthesis of Compound BH1-8

An intermediate M3 was synthesized through a synthesis pathway below.

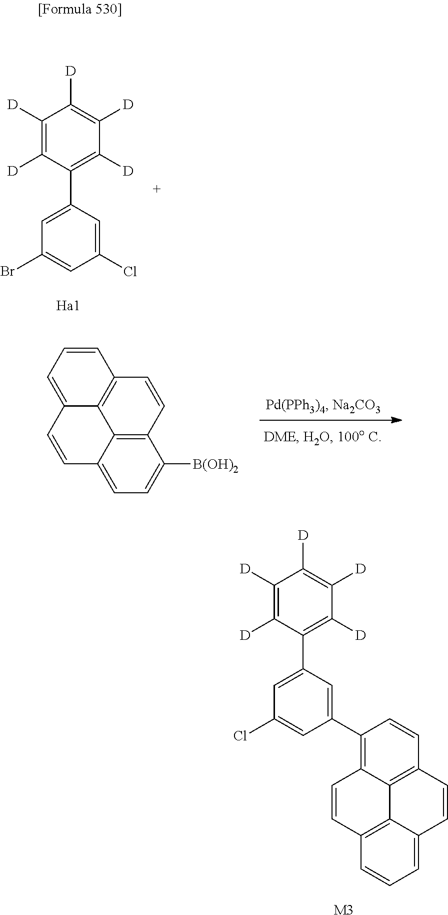

### Synthesis of Intermediate M3

Under argon atmosphere, 1.05 g of a halogen intermediate Hal, 0.95 g of pyrene-1-ylboronic acid synthesized by a known method, 0.13 g of tetrakis(triphenylphosphine)palladium(0) (Pd(PPh3)₄), 0.82 g of sodium carbonate, 8 mL of DME, and 4 mL of ion-exchange water were put into a flask and stirred for 18 hours at 100 degrees C. After being stirred, the obtained solution was cooled to the room temperature. After the solution was cooled, an organic layer was washed with saturated saline solution and then added with sodium sulfate to be dried. After the drying, filtration and concentration under reduced pressure were conducted. The crude product was purified by silica-gel chromatography using a mixed solvent of toluene and hexane to obtain 1.40 g of a light yellow solid (yield of 92%). As a result of FD-MS analysis, the light yellow solid was the intermediate M3 and m/e was equal to 394 while a molecular weight was 393.92.

An intermediate M4 was synthesized through a synthesis pathway below.

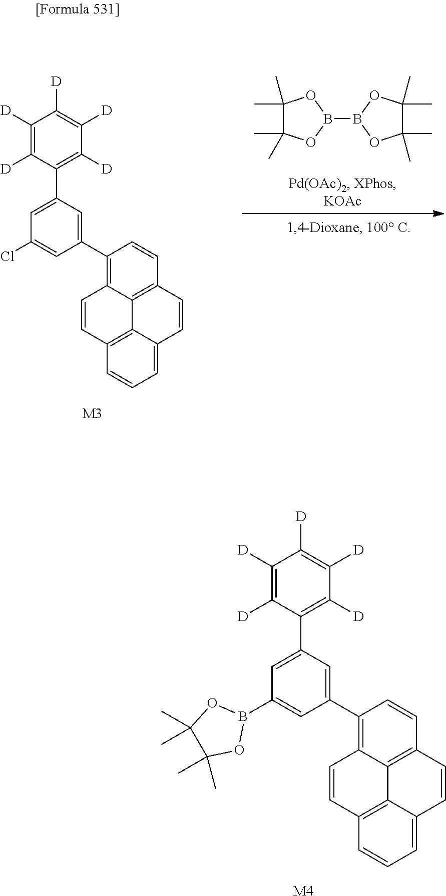

### Synthesis of Intermediate M4

Under argon atmosphere, 1.93 g of the intermediate M3 (chloro intermediate), 2.00 g of bis(pinacolato)diboron, 0.02 g of palladium acetate(II), 0.50 g of potassium acetate, and 6 mL of 1,4-dioxane were put into a flask and stirred for seven hours at 100 degrees C. After being stirred, the obtained solution was cooled to the room temperature. After the solution was cooled, an organic layer was washed with saturated saline solution and then added with sodium sulfate to be dried. After the drying, filtration and concentration under reduced pressure were conducted. The crude product was purified by silica-gel chromatography using toluene to obtain 1.00 g of a light yellow solid (yield of 81%). As a result of FD-MS analysis, the light yellow solid was the intermediate M4 and m/e was equal to 486 while a molecular weight was 485.44.

A compound BH1-8 was synthesized through a synthesis pathway below.

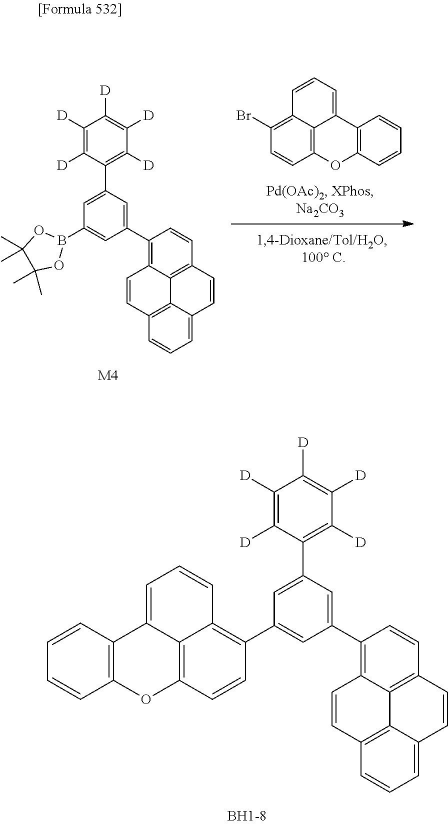 [SMILES: [2H]c1c([2H])c([2H])c(-c2cc(B3OC(C)(C)C(C)(C)O3)cc(-c3ccc4ccc5cccc6ccc3c4c56)c2)c([2H])c1[2H] ; Brc1ccc2c3c(cccc13)-c1ccccc1O2 ; "M4" ; [2H]c1c([2H])c([2H])c(-c2cc(-c3ccc4c5c(cccc35)-c3ccccc3O4)cc(-c3ccc4ccc5cccc6ccc3c4c56)c2)c([2H])c1[2H]]

### Synthesis of Compound BH1-8

Under argon atmosphere, 25.0 g of the intermediate M4 (boronic acid pinacol intermediate), 15.3 g of a bromo intermediate synthesized by a known method, 2.98 g of tetrakis(triphenylphosphine)palladium(0) (Pd(PPh3)₄), 13.8 g of sodium carbonate, 520 mL of 1,4-dioxane, and 200 mL of ion-exchange water were put into a flask and refluxed for 18 hours with stirring. After the reflux with stirring, the obtained solution was cooled to the room temperature. After the solution was cooled, the deposited solid was filtered and collected. The obtained solid was sequentially washed with water and acetone, and then recrystallized with a mixed solvent of toluene and hexane to obtain 11.0 g of a light yellow solid (yield of 37%). As a result of FD-MS analysis, the light yellow solid was the compound BH1-8 and m/e was equal to 575 while a molecular weight was 575.72.

### Synthesis Example 9: Synthesis of Compound BH1-9

A compound BH1-9 was synthesized through a synthesis pathway below.

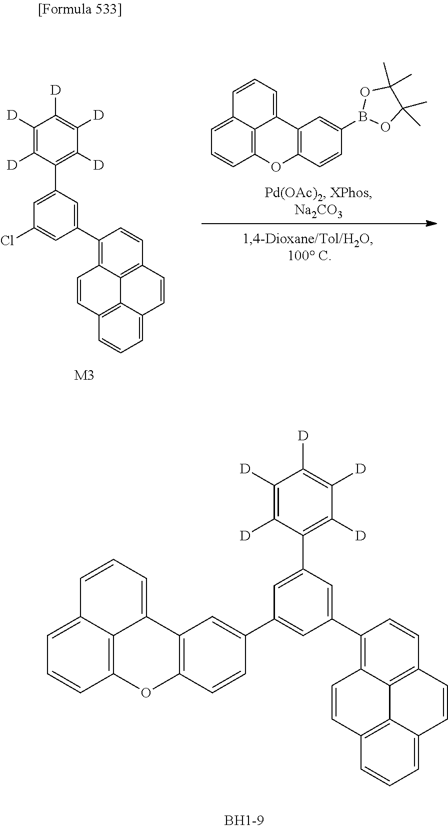 [SMILES: [2H]c1c([2H])c([2H])c(-c2cc(Cl)cc(-c3ccc4ccc5cccc6ccc3c4c56)c2)c([2H])c1[2H] ; CC1(C)OB(c2ccc3c(c2)-c2cccc4cccc(c24)O3)OC1(C)C ; "M3" ; [2H]c1c([2H])c([2H])c(-c2cc(-c3ccc4c(c3)-c3cccc5cccc(c35)O4)cc(-c3ccc4ccc5cccc6ccc3c4c56)c2)c([2H])c1[2H]]

### Synthesis of Compound BH1-9

Under argon atmosphere, 60.0 g of the intermediate M3 (chloro intermediate) synthesized in the same manner as in Synthesis Example 8, 55.1 g of a boronic acid pinacol intermediate synthesized by a known method, 1.03 g of palladium(II) acetate, 32.3 g of sodium carbonate, 450 mL of toluene, 450 mL of 1,4-dioxane, and 170 mL of ion-exchange water were put into a flask and refluxed for six hours with stirring. After the reflux with stirring, the obtained solution was cooled to the room temperature. After the solution was cooled, the deposited solid was filtered and collected. The obtained solid was sequentially washed with water and acetone, and then recrystallized with a mixed solvent of toluene and hexane to obtain 66.9 g of a light yellow solid (yield of 76%). As a result of FD-MS analysis, the light yellow solid was the compound BH1-9 and m/e was equal to 576 while a molecular weight was 575.72.

### Synthesis Example 10: Synthesis of Compound BH1-10

A compound BH1-10 was synthesized through a synthesis pathway below.

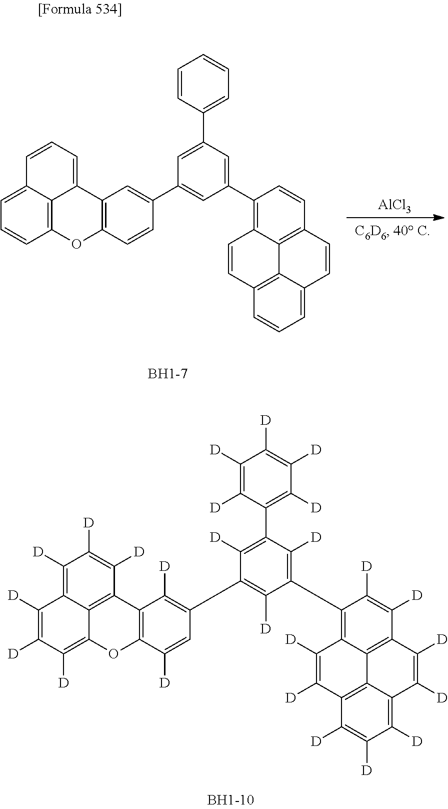 [SMILES: c1ccc(-c2cc(-c3ccc4c(c3)-c3cccc5cccc(c35)O4)cc(-c3ccc4ccc5cccc6ccc3c4c56)c2)cc1 ; "AlCl3" ; [2H]c1cc(Cc2c([2H])c(Cc3c([2H])c([2H])c4c([2H])c([2H])c5c([2H])c([2H])c([2H])c6c([2H])c([2H])c3c4c56)c([2H])c(-c3c([2H])c([2H])c([2H])c([2H])c3[2H])c2[2H])c([2H])c2c1Oc1c([2H])c([2H])c([2H])c3c([2H])c([2H])c([2H])c-2c13]

### Synthesis of Compound BH1-10 (Deuterium Form)

Under argon atmosphere, 20.0 g of the compound BH1-7 (protium form) as a non-deuterated aromatic compound, 4.67 g of aluminum chloride, 1200 mL of benzene d6 were put into a flask and refluxed at 40 degrees C. for 29 hours with stirring. After the reflux with stirring, the obtained solution was cooled to the room temperature. After the solution was cooled, the deposited solid was filtered and collected. The obtained solid was sequentially washed with water and acetone. The washed solid was recrystallized with a mixed solvent of toluene and hexane to obtain 12.7 g of an orange solid (yield of 61%). As a result of FD-MS analysis, the orange solid was the compound BH1-10 in a form of deuterium (deuterated aromatic compound) and m/e was equal to 595 while a molecular weight was 595.84.

### Synthesis Example 11: Synthesis of Compound BH1-11

A compound BH1-11 was synthesized through a synthesis pathway below.

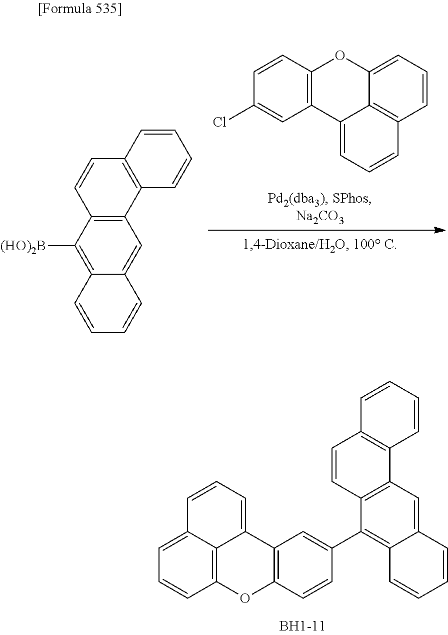 [SMILES: OB(O)c1c2ccccc2cc2c1ccc1ccccc12 ; Clc1ccc2c(c1)-c1cccc3cccc(c13)O2 ; c1ccc2c(-c3ccc4c(c3)-c3cccc5cccc(c35)O4)c3ccc4ccccc4c3cc2c1]

### Synthesis of Compound BH1-11

Under argon atmosphere, 3.79 g of 10-chlorobenzo[kl]xanthene, 8.16 g of tetraphene-7-ylboronic acid synthesized by a known method, 0.41 g of tris(dibenzylideneacetone)dipalladium(0), 1.48 g of 2-dicyclohexylphosphino-2′,6′-dimethoxy-1,1′-biphenyl, 4.77 g of sodium carbonate, 150 mL of 1,4-dioxane, and 20 mL of ion-exchange water were put into a flask and refluxed at 100 degrees C. for six hours with stirring. After the reflux with stirring, the obtained solution was cooled to the room temperature. After the solution was cooled, the deposited solid was filtered and collected. The obtained solid was sequentially washed with water and acetone, and then recrystallized with a mixed solvent of toluene and hexane to obtain 2.10 g of a light yellow solid (yield of 32%). As a result of FD-MS analysis, the light yellow solid was the compound BH1-11 and m/e was equal to 445 while a molecular weight was 444.53.

### Synthesis Example 12: Synthesis of Compound BH1-12

A compound BH- was synthesized through a synthesis pathway below.

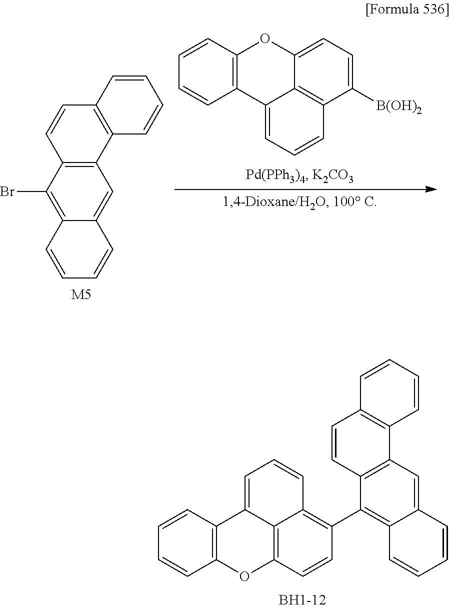

### Synthesis of Compound BH1-12

Under argon atmosphere, 2.40 g of an intermediate M5 (bromo intermediate), 2.37 g of a boronic acid pinacol intermediate synthesized by a known method, 0.18 g of tetrakis(triphenylphosphine)palladium(0), 2.16 g of potassium carbonate, 36 mL of 1,4-dioxane, and 6 mL of ion-exchange water were put into a flask and refluxed at 100 degrees C. for six hours with stirring. After the reflux with stirring, the obtained solution was cooled to the room temperature. After the solution was cooled, the deposited solid was filtered and collected. The obtained solid was sequentially washed with water and acetone, and then recrystallized with a mixed solvent of toluene and hexane to obtain 2.4 g of a light yellow solid (yield of 69%). As a result of FD-MS analysis, the light yellow solid was the compound BH1-12 and m/e was equal to 445 while a molecular weight was 444.53.

### Synthesis Example 13: Synthesis of Compound BH1-13

A compound BH1-13 was synthesized through a synthesis pathway below.

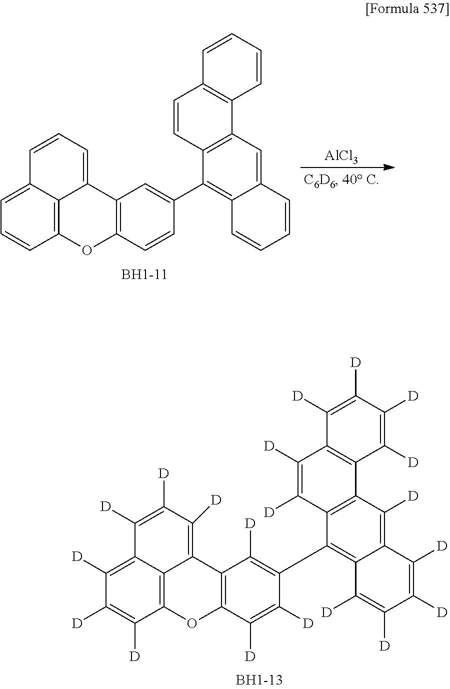

### Synthesis of Compound BH1-13

Under argon atmosphere, 19.0 g of the compound BH1-11 (protium form) as a non-deuterated aromatic compound, 4.6 g of aluminum chloride, and 1140 mL of benzene d₆ were put into a flask and refluxed at 40 degrees C. for 29 hours with stirring. After the reflux with stirring, the obtained solution was cooled to the room temperature. After the solution was cooled, the deposited solid was filtered and collected. The obtained solid was sequentially washed with water and acetone. The washed solid was recrystallized with a mixed solvent of toluene and hexane to obtain 14.0 g of an orange solid (yield of 71%). As a result of FD-MS analysis, the orange solid was the compound BH1-13 as a deuterated aromatic compound and m/e was equal to 463 while a molecular weight was 462.64.

### Synthesis Example 14: Synthesis of Compound BH1-14

A compound BH1-14 was synthesized through a synthesis pathway below.

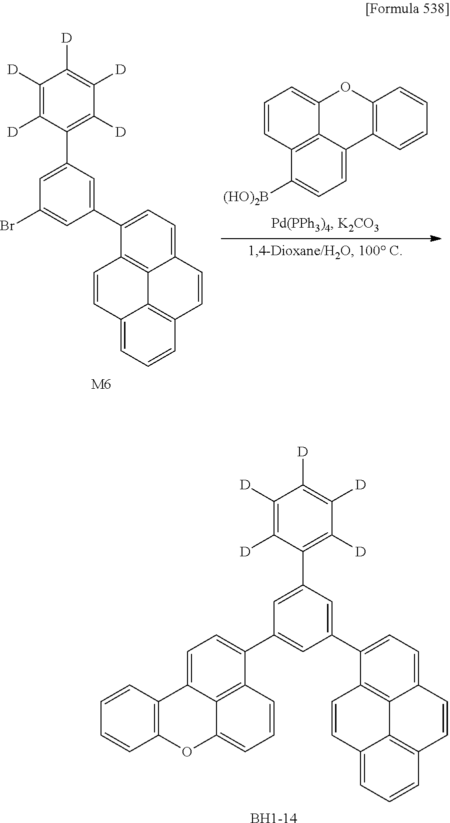

### Synthesis of Compound BH1-14

Under argon atmosphere, 2.60 g of an intermediate M6 (bromo intermediate), 1.55 g of a boronic acid intermediate synthesized by a known method, 0.14 g of tetrakis(triphenylphosphine)palladium(0), 1.64 g of potassium carbonate, 40 mL of 1,4-dioxane, and 10 mL of ion-exchange water were put into a flask and refluxed at 100 degrees C. for six hours with stirring. After the reflux with stirring, the obtained solution was cooled to the room temperature. After the solution was cooled, the deposited solid was filtered and collected. The obtained solid was sequentially washed with water and acetone, and then recrystallized with a mixed solvent of toluene and hexane to obtain 1.70 g of a light yellow solid (yield of 50%). As a result of FD-MS analysis, the light yellow solid was the compound BH1-14 and m/e was equal to 576 while a molecular weight was 575.72.

### Synthesis Example 15: Synthesis of Compound BH1-15

A compound BH1-15 was synthesized through a synthesis pathway below.

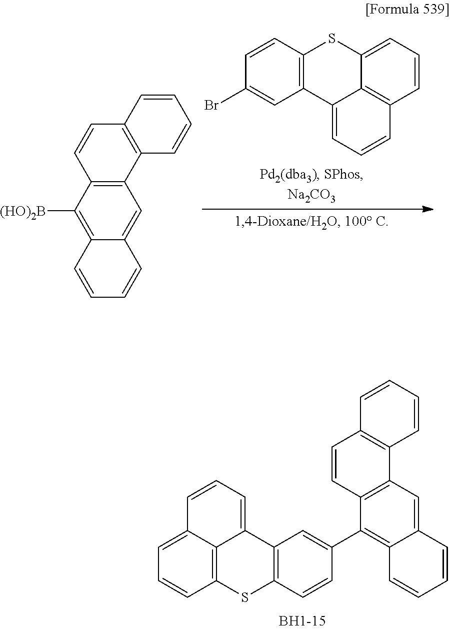

### Synthesis of Compound BH1-15

Under argon atmosphere, 4.62 g of 10-bromobenzo[kl]thioxanthene, 8.16 g of tetraphene-7-ylboronic acid synthesized by a known method, 0.52 g of tris(dibenzylideneacetone)dipalladium(0), 1.63 g of 2-dicyclohexylphosphino-2′,6′-dimethoxy-1,1′-biphenyl, 4.99 g of sodium carbonate, 150 mL of 1,4-dioxane, and 20 mL of ion-exchange water were put into a flask and refluxed at 100 degrees C. for six hours with stirring. After the reflux with stirring, the obtained solution was cooled to the room temperature. After the solution was cooled, the deposited solid was filtered and collected. The obtained solid was sequentially washed with water and acetone, and then recrystallized with a mixed solvent of toluene and hexane to obtain 3.26 g of a light yellow solid (yield of 48%). As a result of FD-MS analysis, the light yellow solid was the compound BH1-15 and m/e was equal to 461 while a molecular weight was 460.59.

### Synthesis Example 16: Synthesis of Compound BH1-16

A compound BH1-16 was synthesized through a synthesis pathway below.

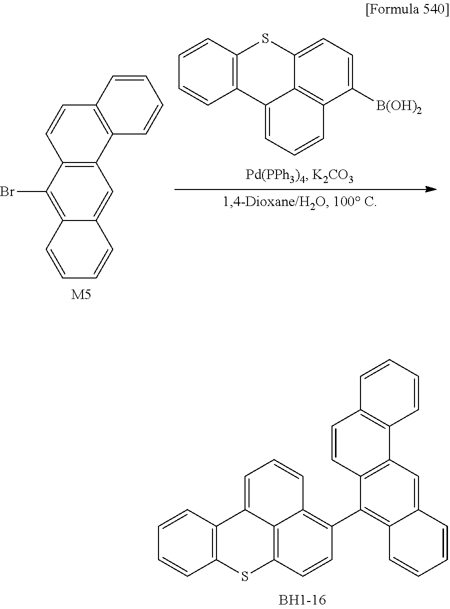

### Synthesis of Compound BH1-16

Under argon atmosphere, 2.40 g of the intermediate M5 (bromo intermediate), 2.37 g of a boronic acid intermediate synthesized by a known method, 0.21 g of tetrakis(triphenylphosphine)palladium(0), 2.33 g of potassium carbonate, 40 mL of 1,4-dioxane, and 10 mL of ion-exchange water were put into a flask and refluxed at 100 degrees C. for six hours with stirring. After the reflux with stirring, the obtained solution was cooled to the room temperature. After the solution was cooled, the deposited solid was filtered and collected. The obtained solid was sequentially washed with water and acetone, and then recrystallized with a mixed solvent of toluene and hexane to obtain 2.16 g of a light yellow solid (yield of 55%). As a result of FD-MS analysis, the light yellow solid was the compound BH1-16 and m/e was equal to 461 while a molecular weight was 460.59.

### Synthesis Example 17: Synthesis of Compound BH1-17

A compound BH1-17 was synthesized through a synthesis pathway below.

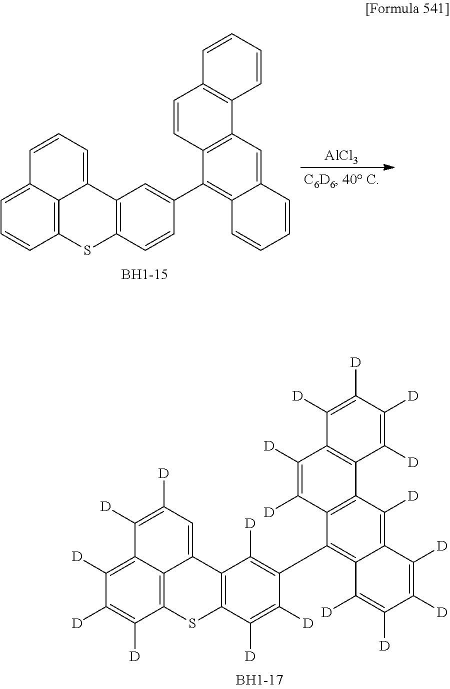

### Synthesis of Compound BH1-17

Under argon atmosphere, 19.0 g of the compound BH1-15 (protium form) as a non-deuterated aromatic compound, 4.6 g of aluminum chloride, and 1140 mL of benzene d6 were put into a flask and refluxed at 40 degrees C. for 29 hours with stirring. After the reflux with stirring, the obtained solution was cooled to the room temperature. After the solution was cooled, the deposited solid was filtered and collected. The obtained solid was sequentially washed with water and acetone. The washed solid was recrystallized with a mixed solvent of toluene and hexane to obtain 14.6 g of an orange solid (yield of 74%). As a result of FD-MS analysis, the orange solid was the compound BH1-17 as a deuterated aromatic compound and m/e was equal to 480 while a molecular weight was 479.71.

### Synthesis Example 18: Synthesis of Compound BH1-18

A compound BH1-18 was synthesized through a synthesis pathway below.

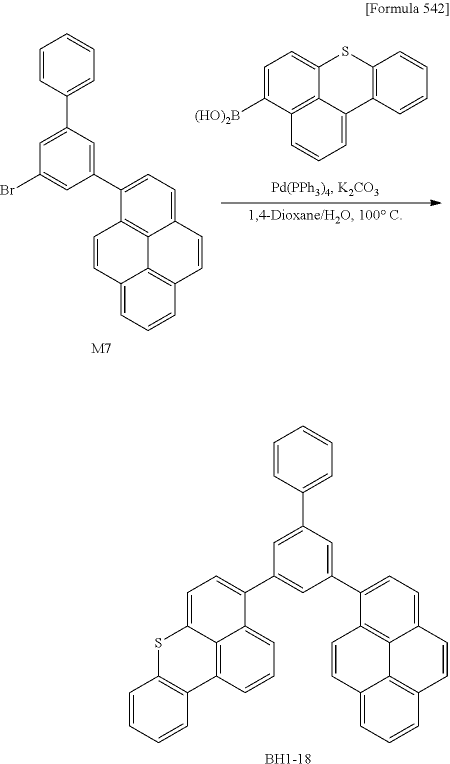

### Synthesis of Compound BH1-18

Under argon atmosphere, 2.60 g of an intermediate M7 (bromo intermediate), 2.05 g of a boronic acid intermediate synthesized by a known method, 0.22 g of tetrakis(triphenylphosphine)palladium(0), 1.68 g of potassium carbonate, 50 mL of 1,4-dioxane, and 10 mL of ion-exchange water were put into a flask and refluxed at 100 degrees C. for six hours with stirring. After the reflux with stirring, the obtained solution was cooled to the room temperature. After the solution was cooled, the deposited solid was filtered and collected. The obtained solid was sequentially washed with water and acetone, and then recrystallized with a mixed solvent of toluene and hexane to obtain 2.32 g of a light yellow solid (yield of 66%). As a result of FD-MS analysis, the light yellow solid was the compound BH1-18 and m/e was equal to 587 while a molecular weight was 586.75.

### Synthesis Example 19: Synthesis of Compound BH1-19

A compound BH1-19 was synthesized through a synthesis pathway below.

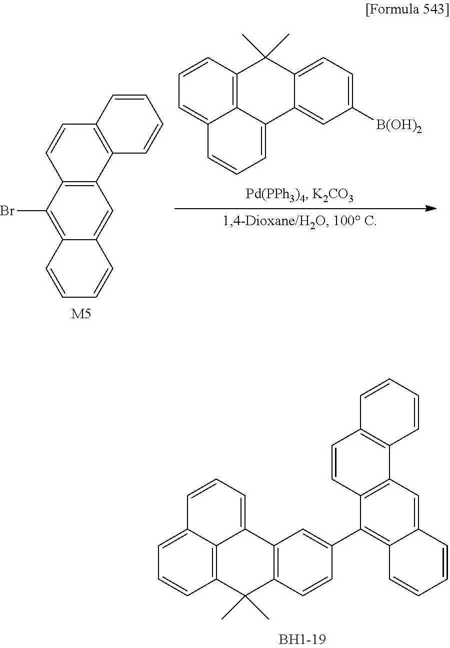

### Synthesis of Compound BH1-19

Under argon atmosphere, 2.30 g of the intermediate M5 (bromo intermediate), 2.37 g of a boronic acid intermediate synthesized by a known method, 0.23 g of tetrakis(triphenylphosphine)palladium(0), 2.23 g of potassium carbonate, 40 mL of 1,4-dioxane, and 10 mL of ion-exchange water were put into a flask and refluxed at 100 degrees C. for six hours with stirring. After the reflux with stirring, the obtained solution was cooled to the room temperature. After the solution was cooled, the deposited solid was filtered and collected. The obtained solid was sequentially washed with water and acetone, and then recrystallized with a mixed solvent of toluene and hexane to obtain 2.40 g of a light yellow solid (yield of 64%). As a result of FD-MS analysis, the light yellow solid was the compound BH1-19 and m/e was equal to 471 while a molecular weight was 470.62.

### Synthesis Example 20: Synthesis of Compound BH1-20

A compound BH1-20 was synthesized through a synthesis pathway below.

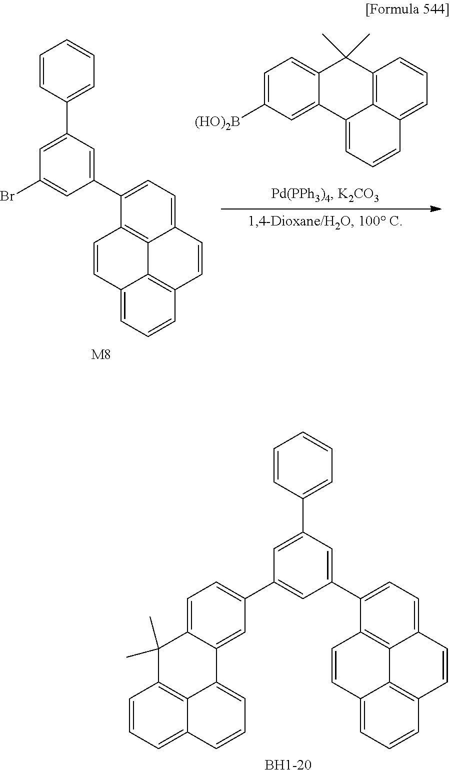

### Synthesis of Compound BH1-20

Under argon atmosphere, 2.60 g of an intermediate M8 (bromo intermediate), 2.02 g of a boronic acid intermediate synthesized by a known method, 0.25 g of tetrakis(triphenylphosphine)palladium(0), 1.67 g of potassium carbonate, 50 mL of 1,4-dioxane, and 10 mL of ion-exchange water were put into a flask and refluxed at 100 degrees C. for six hours with stirring. After the reflux with stirring, the obtained solution was cooled to the room temperature. After the solution was cooled, the deposited solid was filtered and collected. The obtained solid was sequentially washed with water and acetone, and then recrystallized with a mixed solvent of toluene and hexane to obtain 2.89 g of a light yellow solid (yield of 69%). As a result of FD-MS analysis, the light yellow solid was the compound BH1-20 and m/e was equal to 597 while a molecular weight was 596.77.

### Synthesis Example 21: Synthesis of Compound BH1-21

An intermediate M9 was synthesized through a synthesis pathway below.

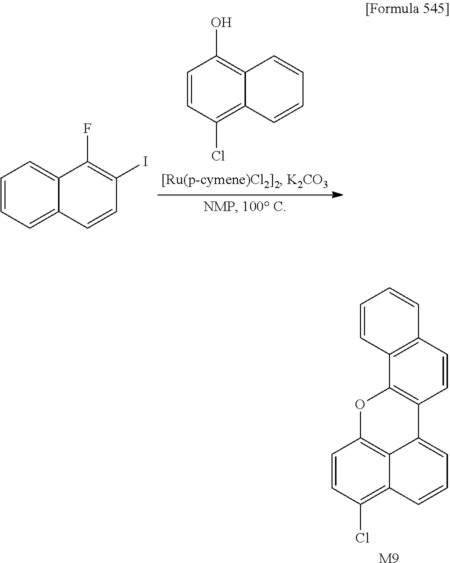

### Synthesis of Intermediate M9

Under argon atmosphere, 10.0 g of 1-fluoro-2-iodonaphthalene, 6.57 g of 4-chloro-1-naphthol, 0.90 g of dichloro(p-cymene)ruthenium(II) (dimer), 7.21 g of potassium carbonate, and 200 mL of N-methyl-2-pyrrolidone were put into a flask and refluxed at 100 degrees C. for six hours with stirring. After the reflux with stirring, the obtained solution was cooled to the room temperature. After the solution was cooled, the deposited solid was filtered and collected. The obtained solid was sequentially washed with water and acetone, and then recrystallized with a mixed solvent of toluene and hexane to obtain 1.20 g of a light yellow solid (yield of 11%). As a result of FD-MS analysis, the light yellow solid was the intermediate M9 and m/e was equal to 303.

A compound BH1-21 was synthesized through a synthesis pathway below.

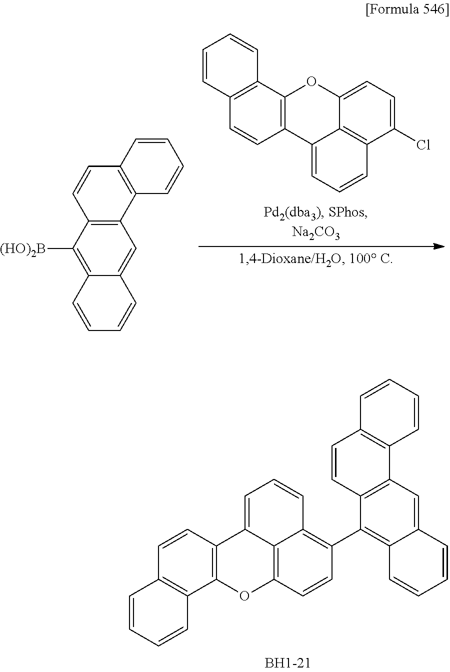 [SMILES: OB(O)c1c2ccccc2cc2c1ccc1ccccc12 ; Clc1ccc2c3c(cccc13)-c1ccc3ccccc3c1O2 ; c1ccc2c3c(ccc2c1)-c1cccc2c(-c4c5ccccc5cc5c4ccc4ccccc45)ccc(c12)O3]

### Synthesis of Compound BH1-21

Under argon atmosphere, 4.88 g of the intermediate M9, 8.16 g of tetraphene-7-yl boronic acid synthesized by a known method, 0.62 g of tris(dibenzylideneacetone)dipalladium(0), 1.77 g of 2-dicyclohexylphosphino-2′,6′-dimethoxy-1,1′-biphenyl, 5.11 g of sodium carbonate, 150 mL of 1,4-dioxane, and 20 mL of ion-exchange water were put into a flask and refluxed at 100 degrees C. for six hours with stirring. After the reflux with stirring, the obtained solution was cooled to the room temperature. After the solution was cooled, the deposited solid was filtered and collected. The obtained solid was sequentially washed with water and acetone, and then recrystallized with a mixed solvent of toluene and hexane to obtain 3.82 g of a light yellow solid (yield of 48%). As a result of FD-MS analysis, the light yellow solid was a compound BH1-21 and m/e was equal to 495 while a molecular weight was 494.59.

### Synthesis Example 22: Synthesis of Compound BH1-22

A compound X-1 was synthesized through a synthesis pathway below.

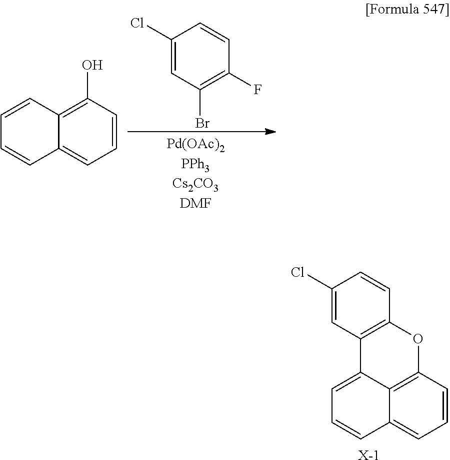

### Synthesis of Compound X-1

1-hydroxynaphthalene (1.4 g), 2-bromo-4-chloro-1-fluorobenzene (2.5 g), diacetoxy palladium (0.11 g), triphenylphosphine (0.53 g), and cesium carbonate (13 g) were added to dimethyl formamide (50 mL). Under argon atmosphere, the obtained solution was heated to 140 degrees C. and stirred for six hours. After the reaction solution was stirred, a solvent was distilled away from the reaction solution under reduced pressure. The obtained residue was subjected to silica-gel column chromatography to obtain 1.2 g of a white solid (yield of 49%). As a result of FD-MS analysis, the white solid was the compound X-1 and m/e was equal to 253 while a molecular weight was 252.70.

A compound X-2 was synthesized through a synthesis pathway below.

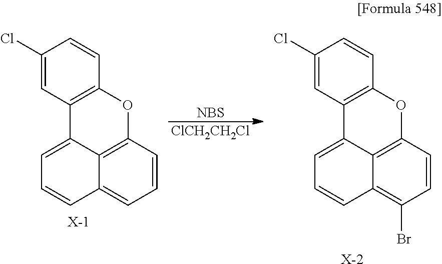

### Synthesis of Compound X-2

The compound X-1 (10 g) was dissolved in 1,2-dichloroethane (200 mL), to which N-bromosuccinimide (NBS) (6.7 g) was added. Under nitrogen atmosphere, the reaction solution was heated to 80 degrees C. and stirred for six hours. After the reaction solution was stirred, a sodium carbonate aqueous solution was added to the reaction solution. The deposited solid was collected by filtration and recrystallized with toluene to obtain 7.2 g of a light yellow solid (yield of 55%). As a result of FD-MS analysis, the light yellow solid was the compound X-2 and m/e was equal to 332 while a molecular weight was 331.59.

A compound X-3 was synthesized through a synthesis pathway below.

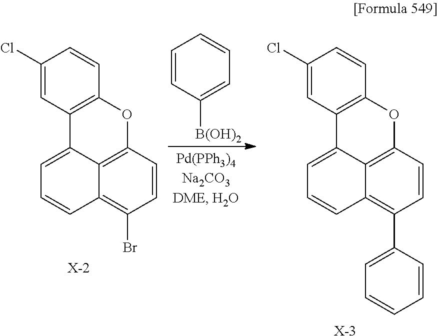

### Synthesis of Compound X-3

The compound X-2 (5.0 g), phenylboronic acid (2.8 g), and tetrakis(triphenylphosphine)palladium (0.5 g) were added to dimethoxyethane (150 mL), to which a sodium carbonate aqueous solution (2.0M, 19 mL) was added. The obtained solution was stirred overnight at 80 degrees C. under nitrogen atmosphere. After the solution was stirred, a solvent was distilled away from the solution under reduced pressure. The obtained residue was subjected to silica-gel column chromatography to obtain 3.9 g of a yellow solid (yield of 78%). As a result of FD-MS analysis, the yellow solid was the compound X-3 and m/e was equal to 329 while a molecular weight was 328.80.

A compound BH1-22 was synthesized through a synthesis pathway below.

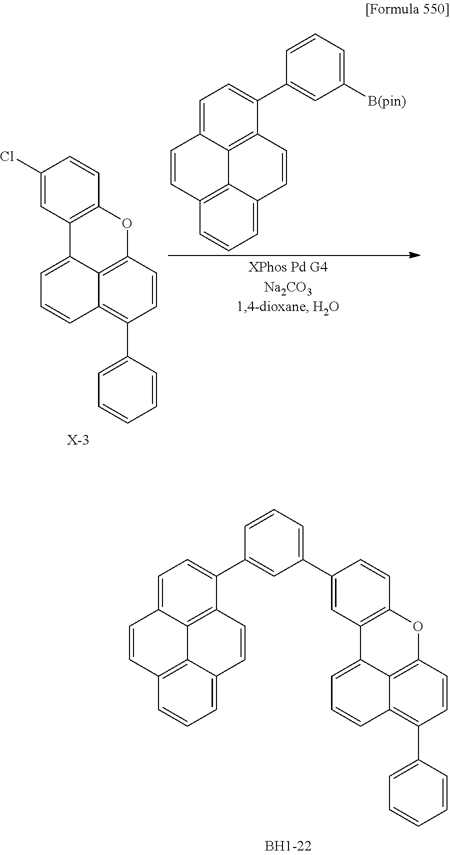

### Synthesis of Compound BH1-22

The compound X-3 (0.8 g), 4,4,5,5-tetramethyl-2-[3-(1-pyrenyl)phenyl]-1,3,2-dioxaborolane (1.0 g), and XPhos Pd G4 (0.1 g) were dissolved in 1,4-dioxane (13 mL), to which a sodium carbonate aqueous solution (2.0M, 3.8 mL) was added. Under argon atmosphere, the obtained solution was heated to 100 degrees C. and stirred for one hour.

After being stirred, the reaction solution was filtered to obtain a solid. The solid was recrystallized with 1,4-dioxane to obtain 1.0 g of a light yellow solid (yield of 71%). As a result of FD-MS analysis, the light yellow solid was the compound BH1-22 and m/e was equal to 571 while a molecular weight was 570.69.

### Synthesis Example 23: Synthesis of Compound BH1-23

A compound P-1 was synthesized through a synthesis pathway below.

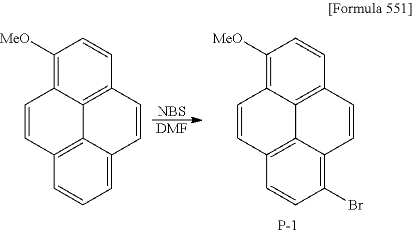

### Synthesis of Compound P-1

1-methoxypyrene (5.0 g) was dissolved in N,N-dimethylformamide (DMF) (100 mL) and cooled to 0 degree C. in an ice bath. N-bromosuccinimide (NBS) (3.6 g) was added to this solution. The obtained solution was stirred for two hours, and subsequently stirred for another two hours at the room temperature. After being stirred, the reaction solution was added with water to deposit a solid. The solid was collected by filtration to obtain 6.3 g of a colorless solid (yield of 94%). As a result of FD-MS analysis, the colorless solid was the compound P-1 and m/e was equal to 311 while a molecular weight was 311.18.

A compound P-2 was synthesized through a synthesis pathway below.

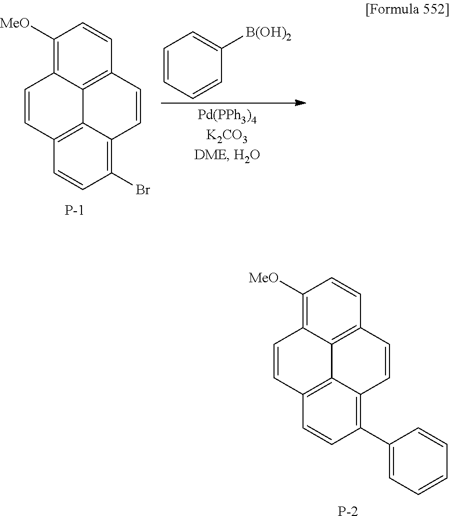

### Synthesis of Compound P-2

The compound P-1 (5.8 g), phenylboronic acid (2.95 g), and tetrakis(triphenylphosphine)palladium (0.64 g) were dissolved in dimethoxyethane (80 mL), to which an aqueous solution (16 mL) of potassium carbonate (5.2 g) was added. Under argon atmosphere, the reaction solution was stirred for eight hours while being heated to 80 degrees C. After the reaction solution was stirred, a solvent was distilled away from the reaction solution under reduced pressure. The obtained residue was subjected to gel filtration chromatography to obtain 4.6 g of a colorless solid (yield of 80%). As a result of FD-MS analysis, the colorless solid was the compound P-2 and m/e was equal to 308 while a molecular weight was 308.38.

A compound P-3 was synthesized through a synthesis pathway below.

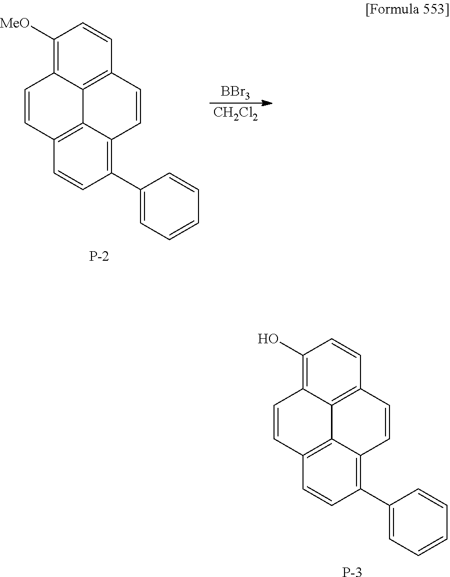

### Synthesis of Compound P-3

The compound P-2 (4.6 g) was dissolved in dichloromethane (100 mL), to which 60 mL of boron tribromide in dichloromethane (1.0 M) was added dropwise under ice-cooling. Subsequently, the obtained solution was stirred at the room temperature for two days. The stirred reaction solution was added to ice water and extracted with ethyl acetate. Subsequently, an organic layer from which a solvent was distilled away was subjected to silica-gel column chromatography to obtain 4.2 g of a colorless solid (yield of 95%). As a result of FD-MS analysis, the colorless solid was the compound P-3 and m/e was equal to 294 while a molecular weight was 294.35.

A compound P-4 was synthesized through a synthesis pathway below.

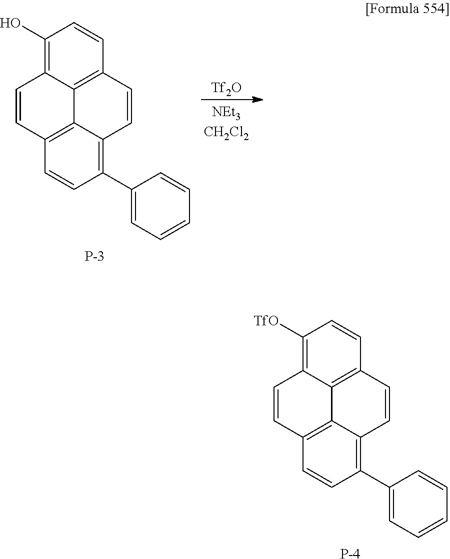

### Synthesis of Compound P-4

The compound P-3 (4.3 g) and trimethylamine (3.0 g) were dissolved in dichloromethane (50 mL), to which trifluoromethanesulfonic anhydride (5.0 g) was added while being cooled in an ice bath. After the reaction solution was stirred for two hours, a solvent was distilled away from the reaction solution under reduced pressure. The obtained residue was subjected to silica-gel column chromatography to obtain 4.3 g of a white solid (yield of 85%). As a result of FD-MS analysis, the white solid was the compound P-4 and m/e was equal to 426 while a molecular weight was 426.41.

A compound P-5 was synthesized through a synthesis pathway below.

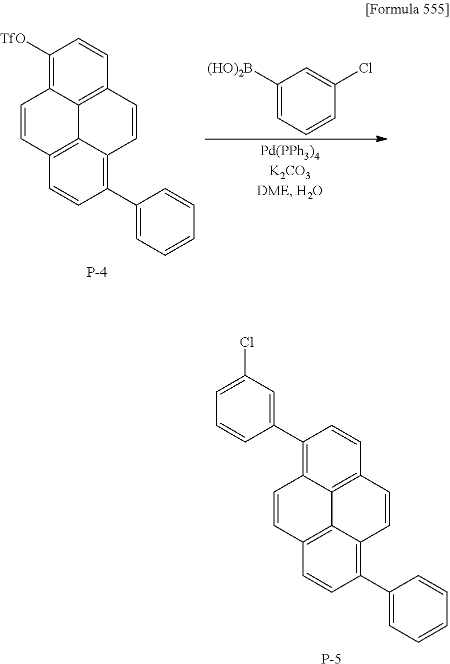

### Synthesis of Compound P-5

The compound P-4 (1.7 g), 3-chlorophenylboronic acid (0.69 g), and tetrakis(triphenylphosphine)palladium (0.23 g) were dissolved in dimethoxyethane (20 mL), to which an aqueous solution (4.0 mL) of potassium carbonate (1.1 g) was added. The obtained solution was stirred at 80 degrees C. for five hours under argon atmosphere. After the reaction solution was stirred, a solvent was distilled away from the reaction solution under reduced pressure. The obtained residue was subjected to gel filtration chromatography to obtain 1.2 g of a white solid (yield of 78%). As a result of FD-MS analysis, the white solid was the compound P-5 and m/e was equal to 389 while a molecular weight was 388.89.

A compound BH1-23 was synthesized through a synthesis pathway below.

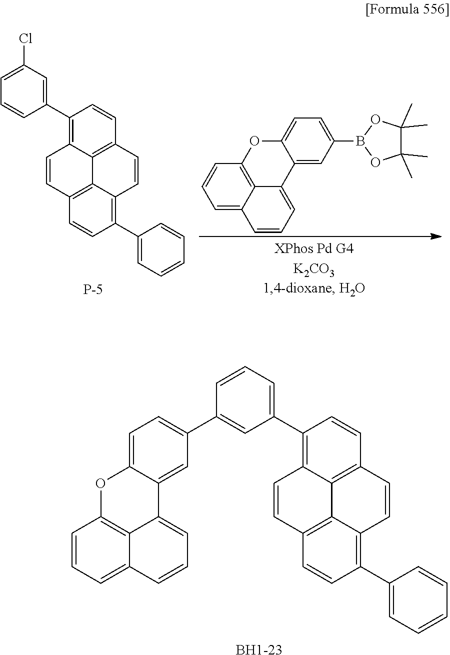

### Synthesis of Compound BH1-23

The compound P-5 (1.2 g), benzo[kl]xanthene-10-yl-boronic acid (3.70 g), and XPhos Pd G4 (0.13 g) were added to and dissolved in 1,4-dioxane (15 mL), to which an aqueous solution (3.0 mL) of potassium carbonate (1.3 g) was added. Under argon atmosphere, the obtained solution was heated for reflux with stirring for two hours. After being stirred, the reaction solution was filtered to obtain a solid. The solid was purified by silica-gel column chromatography to obtain 0.58 g of a white solid (yield of 33%). As a result of FD-MS analysis, the white solid was the compound BH1-23 and m/e was equal to 571 while a molecular weight was 570.69.

### Synthesis Example 24: Synthesis of Compound BH1-24

A compound X-4 was synthesized through a synthesis pathway below.

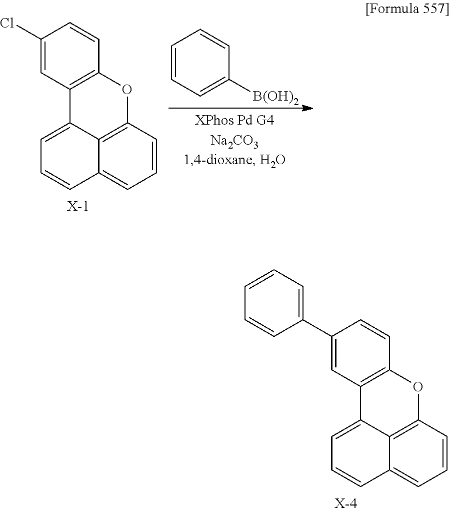

### Synthesis of Compound X-4

The compound X-1 (1.0 g), phenylboronic acid (0.9 g), and XPhos Pd G4 (0.17 g) were added to 1,4-dioxane (50 mL), to which a sodium carbonate aqueous solution (2.0M, 6.0 mL) was added. Under argon atmosphere, the obtained solution was heated at 100 degrees C. for six hours with stirring.

After the reaction solution was stirred, a solvent was distilled away from the reaction solution under reduced pressure. The obtained residue was subjected to silica-gel column chromatography to obtain 0.7 g of a light yellow solid (yield of 60%). As a result of FD-MS analysis, the light yellow solid was the compound X-4 and m/e was equal to 294 while a molecular weight was 294.35.

A compound X-5 was synthesized through a synthesis pathway below.

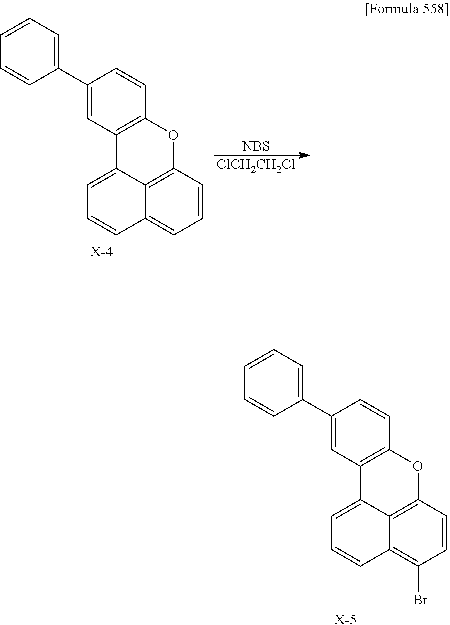

### Synthesis of Compound X-5

The compound X-4 (1.0 g) was dissolved in 1,2-dichloroethane (20 mL), to which N-bromosuccinimide (NBS) (0.61 g) was added. Under nitrogen atmosphere, the reaction solution was heated to 80 degrees C. and stirred for six hours. After the reaction solution was stirred, a sodium carbonate aqueous solution was added to the reaction solution. The deposited solid was collected by filtration and recrystallized with toluene to obtain 0.63 g of a yellow solid (yield of 50%). As a result of FD-MS analysis, the yellow solid was the compound X-5 and m/e was equal to 373 while a molecular weight was 373.25.

A compound BH1-24 was synthesized through a synthesis pathway below.

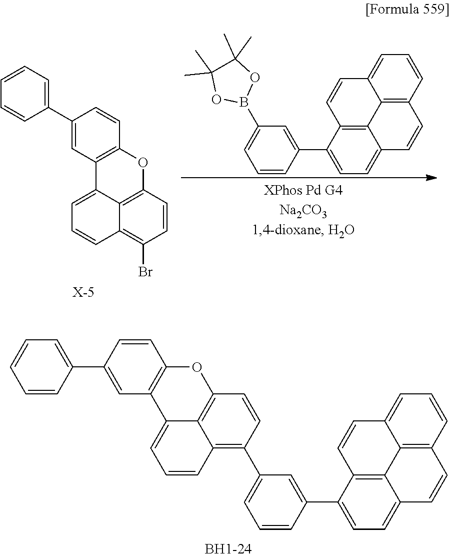 [SMILES: Brc1ccc2c3c(cccc13)-c1cc(-c3ccccc3)ccc1O2 ; CC1(C)OB(c2cccc(-c3ccc4ccc5cccc6ccc3c4c56)c2)OC1(C)C ; "X-5" ; c1ccc(-c2ccc3c(c2)-c2cccc4c(-c5cccc(-c6ccc7ccc8cccc9ccc6c7c89)c5)ccc(c24)O3)cc1]

### Synthesis of Compound BH1-24

The compound X-5 (1.0 g), 4,4,5,5-tetramethyl-2-[3-(1-pyrenyl)phenyl]-1,3,2-dioxaborolane (1.3 g), and XPhos Pd G4 (0.1 g) were dissolved in 1,4-dioxane (20 mL), to which a sodium carbonate aqueous solution (2.0M, 4.0 mL) was added. Under argon atmosphere, the obtained solution was heated to 100 degrees C. and stirred for one hour.

After being stirred, the reaction solution was filtered to obtain a solid. The solid was recrystallized with 1,4-dioxane to obtain 1.0 g of a light yellow solid (yield of 65%). As a result of FD-MS analysis, the light yellow solid was the compound BH1-24 and m/e was equal to 571 while a molecular weight was 570.69.

### Synthesis Example 25: Synthesis of Compound BH1-25

A compound X-6 was synthesized through a synthesis pathway below.

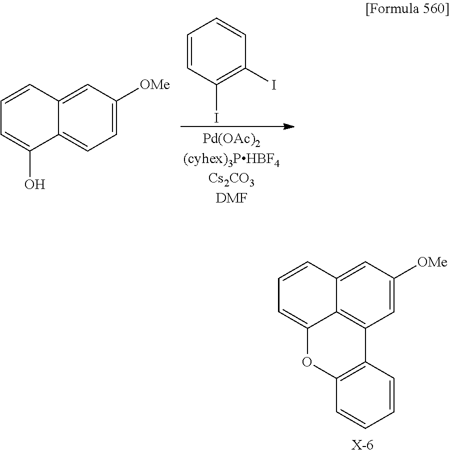

### Synthesis of Compound X-6

6-methoxy-1-naphthol (0.17 g), 1,2-diiodebenzene (0.40 g), palladium acetate (0.02 g), tricyclohexylphosphine tetrafluoroborate (0.07 g), and cesium carbonate (1.17 g) were added to N,N-dimethylformamide (DMF) (10 mL) and stirred at 140 degrees C. for four hours under argon atmosphere. After being stirred, the reaction solution was purified by silica-gel column chromatography to obtain 0.25 g of a colorless solid (yield of 72%). As a result of FD-MS analysis, the colorless solid was the compound X-6 and m/e was equal to 248 while a molecular weight was 248.28.

A compound X-7 was synthesized through a synthesis pathway below.

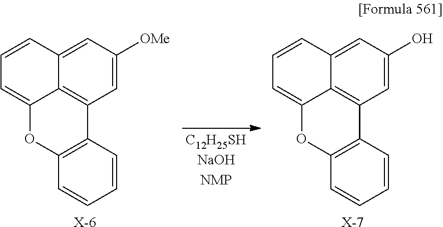

### Synthesis of Compound X-7

The compound X-6 (1.0 g), dodecanethiol (1.2 g), and sodium hydroxide (0.48 g) were added to N-methylpyrrolidone (NMP) (40 mL). The obtained solution was stirred overnight at 150 degrees C. under argon atmosphere. After being stirred, the reaction solution was purified by column chromatography. The obtained crude product was recrystallized with toluene to obtain 0.74 g of a colorless solid (yield of 78%). As a result of FD-MS analysis, the colorless solid was the compound X-7 and m/e was equal to 234 while a molecular weight was 234.25.

A compound X-8 was synthesized through a synthesis pathway below.

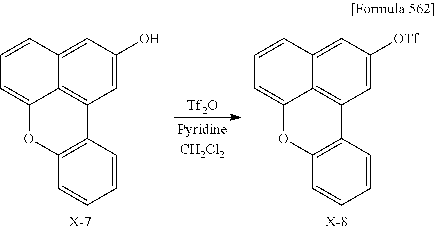

### Synthesis of Compound X-8

The compound X-7 (2.47 g), trifluoromethanesulfonic anhydride (3.57 g), and pyridine (1.00 g) were added to dichloromethane (75 mL) under ice-cooling. The obtained solution was stirred at the room temperature for seven hours under argon atmosphere. After being stirred, the reaction solution was purified by silica-gel column chromatography to obtain 3.47 g of a colorless solid (yield of 90%). As a result of FD-MS analysis, the colorless solid was the compound X-8 and m/e was equal to 366 while a molecular weight was 366.31.

A compound BH1-25 was synthesized through a synthesis pathway below.

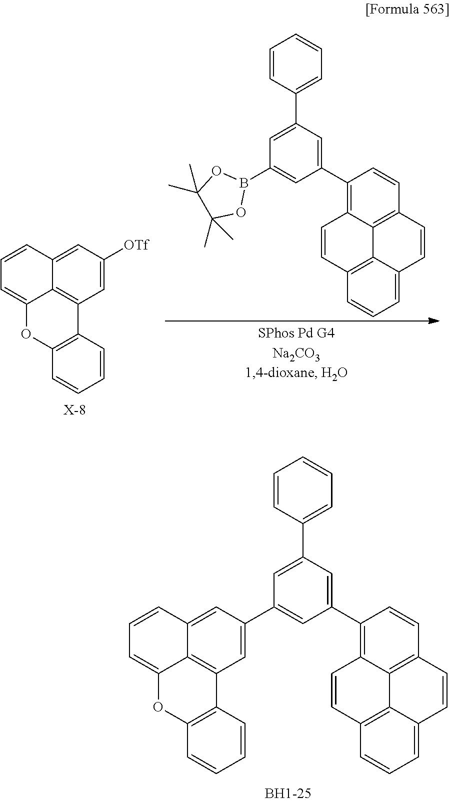

### Synthesis of Compound BH1-25

The compound X-8 (2.0 g), 4,4,5,5-tetramethyl-2-(5-(pyrene-1-yl)-[1,1′-biphenyl]-3-yl)-1,3,2-dioxaborolane (2.6 g), SPhos Pd G4 (0.22 g), and a sodium carbonate aqueous solution (2M, 8.2 mL) were added to 1,4-dioxane (55 mL). Under argon atmosphere, the obtained solution was stirred overnight at 90 degrees C. After the reaction solution was stirred, a solvent was distilled away from the reaction solution under reduced pressure. The obtained residue was recrystallized with toluene to obtain 2.2 g of a light yellow solid (yield of 70%). As a result of FD-MS analysis, the light yellow solid was the compound BH1-25 and m/e was equal to 571 while a molecular weight was 570.69.

### Synthesis Example 26: Synthesis of Compound BH1-26

A compound X-10 was synthesized through a synthesis pathway below.

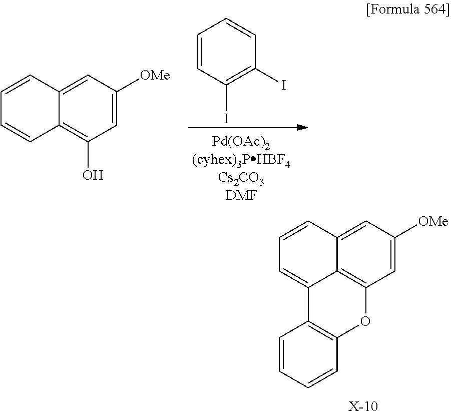

### Synthesis of Compound X-10

3-methoxy-1-naphthol (5.2 g), 1,2-diiodebenzene (12 g), palladium acetate (0.67 g), tricyclohexylphosphine tetrafluoroborate (2.2 g), and cesium carbonate (35 g) were added to N,N-dimethylformamide (DMF) (100 mL) and stirred at 140 degrees C. for six hours under argon atmosphere. After being stirred, the reaction solution was purified by silica-gel column chromatography to obtain 3.7 g of a colorless solid (yield of 50%). As a result of FD-MS analysis, the colorless solid was the compound X-10 and m/e was equal to 248 while a molecular weight was 248.28.

A compound X-11 was synthesized through a synthesis pathway below.

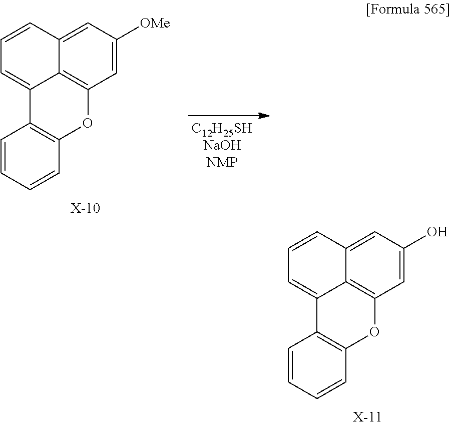

### Synthesis of Compound X-11

The compound X-10 (4.0 g), dodecanethiol (10 g), and sodium hydroxide (3.8 g) were added to N-methylpyrrolidone (NMP) (160 mL). The obtained solution was stirred at 140 degrees C. for five hours under argon atmosphere. After being stirred, the reaction solution was purified by silica-gel column chromatography to obtain 3.56 g of a colorless solid (yield of 95%). As a result of FD-MS analysis, the colorless solid was the compound X-11 and m/e was equal to 234 while a molecular weight was 234.25.

A compound X-12 was synthesized through a synthesis pathway below.

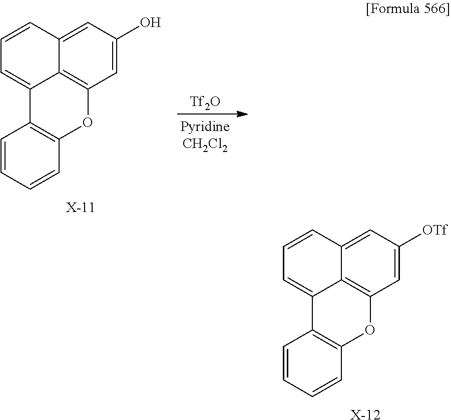

### Synthesis of Compound X-12

The compound X-11 (3.5 g), trifluoromethanesulfonic anhydride (5.2 g), and pyridine (1.4 g) were added to dichloromethane (150 mL) under ice-cooling. The obtained solution was stirred at the room temperature for three hours under argon atmosphere. After being stirred, the reaction solution was purified by silica-gel column chromatography to obtain 1.3 g of a colorless solid (yield of 24%). As a result of FD-MS analysis, the colorless solid was the compound X-12 and m/e was equal to 366 while a molecular weight was 366.31.

A compound BH1-26 was synthesized through a synthesis pathway below.

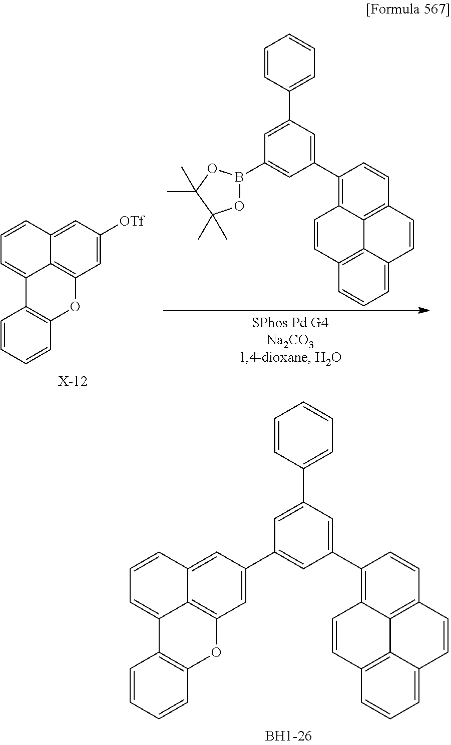

### Synthesis of Compound BH1-26

The compound X-12 (1.2 g), 4,4,5,5-tetramethyl-2-(5-(pyrene-1-yl)-[1,1′-biphenyl]-3-yl)-1,3,2-dioxaborolane (1.5 g), SPhos Pd G4 (0.12 g), and a sodium carbonate aqueous solution (2M, 3.8 mL) were added to 1,4-dioxane (30 mL). Under argon atmosphere, the obtained solution was stirred at 90 degrees C. for seven hours. After the reaction solution was stirred, a solvent was distilled away from the reaction solution under reduced pressure. The obtained residue was recrystallized with toluene to obtain 0.77 g of a light yellow solid (yield of 45%). As a result of FD-MS analysis, the light yellow solid was the compound BH1-26 and m/e was equal to 571 while a molecular weight was 570.69.

### Synthesis Example 27: Synthesis of Compound BH1-27

A compound X-14 was synthesized through a synthesis pathway below.

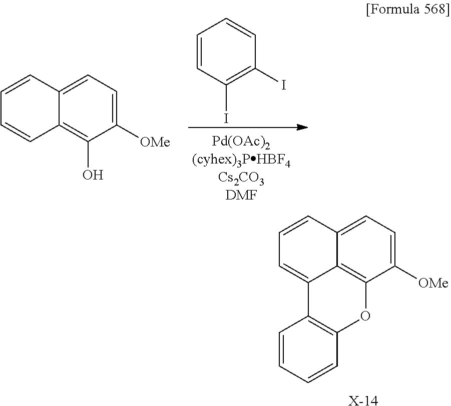

### Synthesis of Compound X-14

2-methoxy-1-naphthol (1.7 g), 1,2-diiodebenzene (4.0 g), palladium acetate (0.23 g), tricyclohexylphosphine tetrafluoroborate (0.74 g), and cesium carbonate (12 g) were added to N,N-dimethylformamide (DMF) (33 mL) and stirred at 140 degrees C. for seven hours under argon atmosphere. After being stirred, the reaction solution was purified by silica-gel column chromatography to obtain 2.2 g of a colorless solid (yield of 45%). As a result of FD-MS analysis, the colorless solid was the compound X-14 and m/e was equal to 248 while a molecular weight was 248.28.

A compound X-15 was synthesized through a synthesis pathway below.

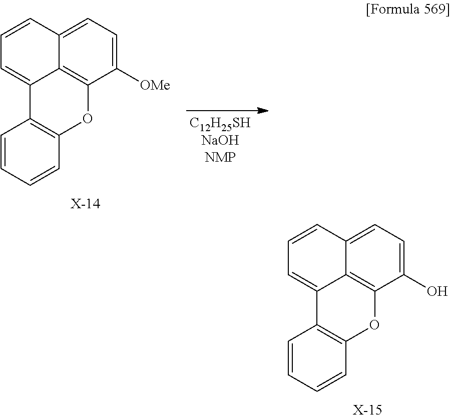

### Synthesis of Compound X-15

The compound X-14 (2.2 g), dodecanethiol (5.7 g), and sodium hydroxide (2.2 g) were added to N-methylpyrrolidone (NMP) (90 mL). The obtained solution was stirred at 140 degrees C. for five hours under argon atmosphere. After being stirred, the reaction solution was purified by silica-gel column chromatography to obtain 1.4 g of a colorless solid (yield of 70%). As a result of FD-MS analysis, the colorless solid was the compound X-15 and m/e was equal to 234 while a molecular weight was 234.25.

A compound X-16 was synthesized through a synthesis pathway below.

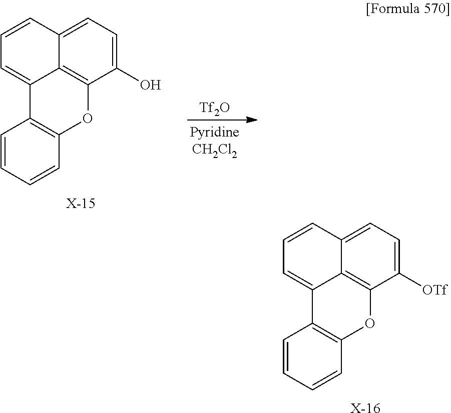

### Synthesis of Compound X-16

The compound X-15 (1.4 g), trifluoromethanesulfonic anhydride (2.1 g), and pyridine (0.57 g) were added to dichloromethane (60 mL) under ice-cooling. The obtained solution was stirred at the room temperature for four hours under argon atmosphere. After being stirred, the reaction solution was purified by silica-gel column chromatography to obtain 1.6 g of a colorless solid (yield of 71%). As a result of FD-MS analysis, the colorless solid was the compound X-16 and m/e was equal to 366 while a molecular weight was 366.31.

A compound BH1-27 was synthesized through a synthesis pathway below.

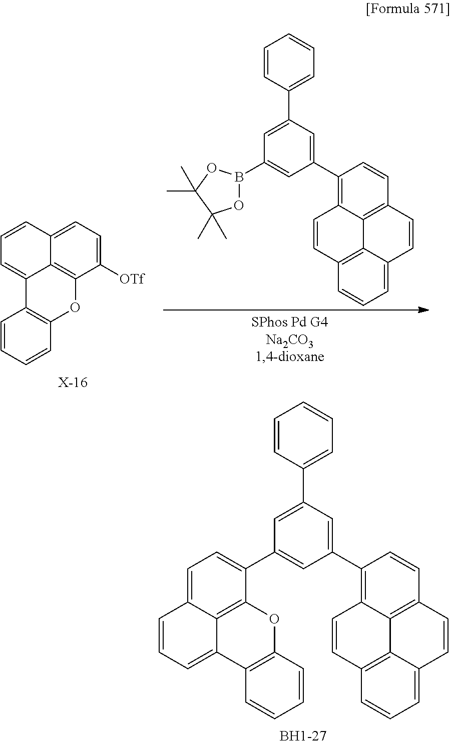

### Synthesis of Compound BH1-27

The compound X-16 (1.4 g), 4,4,5,5-tetramethyl-2-(5-(pyrene-1-yl)-[1,1′-biphenyl]-3-yl)-1,3,2-dioxaborolane (1.7 g), SPhos Pd G4 (0.28 g), and a sodium carbonate aqueous solution (2M, 4.4 mL) were added to 1,4-dioxane (55 mL). Under argon atmosphere, the obtained solution was stirred at 100 degrees C. for seven hours. After the reaction solution was stirred, a solvent was distilled away from the reaction solution under reduced pressure. The obtained residue was recrystallized with toluene to obtain 0.92 g of a light yellow solid (yield of 46%). As a result of FD-MS analysis, the light yellow solid was the compound BH1-27 and m/e was equal to 571 while a molecular weight was 570.69.

### Synthesis Example 28: Synthesis of Compound BH1-28

A compound X-9 was synthesized through a synthesis pathway below.

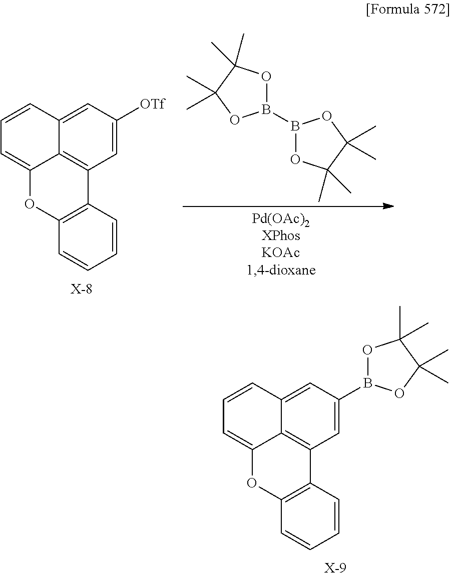

### Synthesis of Compound X-9

The compound X-8 (2.4 g), bis(pinacolato)diboron (3.5 g), palladium acetate (0.1 g), XPhos (0.4 g), and potassium acetate (2.7 g) were added to 1,4-dioxane (50 mL). Under argon atmosphere, the obtained solution was stirred at 100 degrees C. for six hours. After being stirred, the reaction solution was purified by silica-gel column chromatography to obtain 2.0 g of a colorless solid (yield of 89%). As a result of FD-MS analysis, the colorless solid was the compound X-9 and m/e was equal to 344 while a molecular weight was 344.22.

A compound BH1-28 was synthesized through a synthesis pathway below.

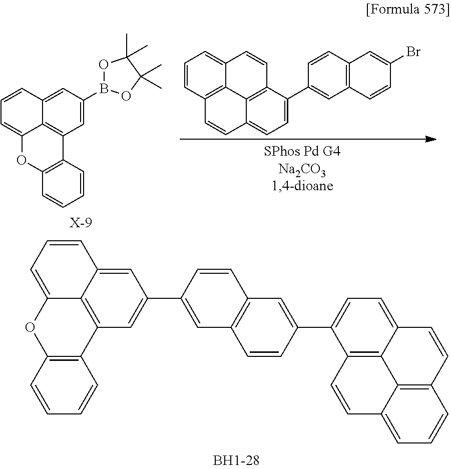

### Synthesis of Compound BH1-28

The compound X-9 (1.2 g), 1-(6-bromonaphthalene-2-yl)-pyrene (1.3 g), SPhos Pd G4 (0.15 g), and a sodium carbonate aqueous solution (2M, 5.2 mL) were added to 1,4-dioxane (40 mL). Under argon atmosphere, the obtained solution was stirred overnight at 90 degrees C. After the reaction solution was stirred, a solvent was distilled away from the reaction solution under reduced pressure. The obtained residue was recrystallized with toluene to obtain 0.85 g of a yellow solid (yield of 45%). As a result of FD-MS analysis, the yellow solid was the compound BH1-28 and m/e was equal to 545 while a molecular weight was 544.65.

### Synthesis Example 29: Synthesis of Compound BH1-29

A compound X-13 was synthesized through a synthesis pathway below.

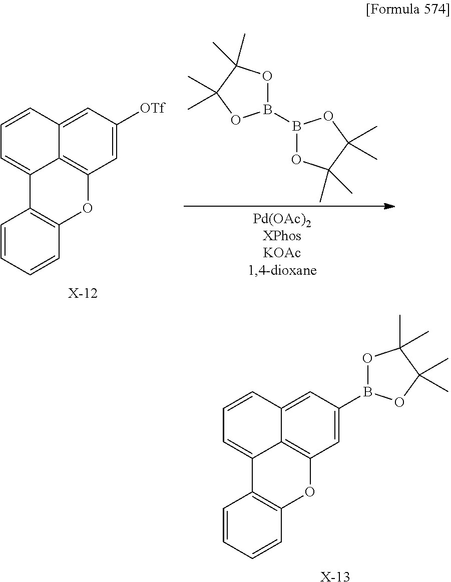

### Synthesis of Compound X-13

The compound X-12 (2.2 g), bis(pinacolato)diboron (3.1 g), palladium acetate (0.1 g), XPhos (0.3 g), and potassium acetate (1.8 g) were added to 1,4-dioxane (50 mL). Under argon atmosphere, the obtained solution was stirred at 100 degrees C. for six hours. After being stirred, the reaction solution was purified by silica-gel column chromatography to obtain 1.8 g of a colorless solid (yield of 87%). As a result of FD-MS analysis, the colorless solid was the compound X-13 and m/e was equal to 344 while a molecular weight was 344.22.

A compound BH1-29 was synthesized through a synthesis pathway below.

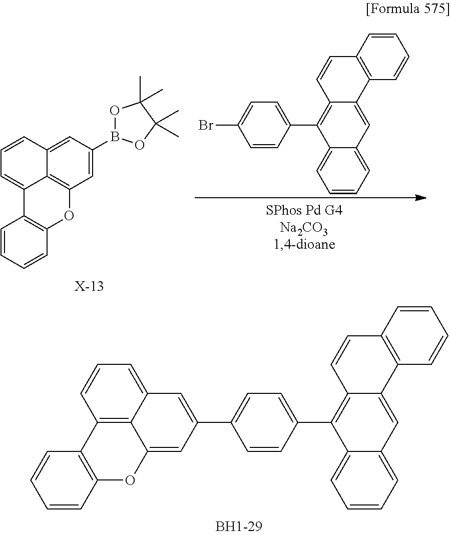

### Synthesis of Compound BH1-29

The compound X-13 (3.1 g), 7-(4-bromophenyl)-tetraphene (3.5 g), SPhos Pd G4 (0.32 g), and a sodium carbonate aqueous solution (2M, 15 mL) were added to 1,4-dioxane (100 mL). Under argon atmosphere, the obtained solution was stirred at 90 degrees C. for five hours. After the reaction solution was stirred, a solvent was distilled away from the reaction solution under reduced pressure. The obtained residue was recrystallized with toluene to obtain 3.1 g of a light yellow solid (yield of 66%). As a result of FD-MS analysis, the light yellow solid was the compound BH1-29 and m/e was equal to 521 while a molecular weight was 520.63.

### Synthesis Example 30: Synthesis of Compound BH1-30

A compound X-17 was synthesized through a synthesis pathway below.

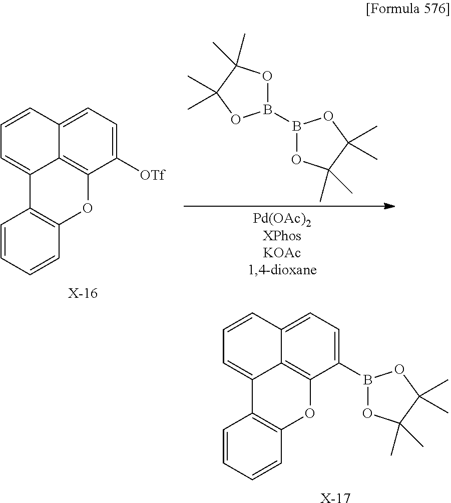

### Synthesis of Compound X-17

The compound X-16 (1.5 g), bis(pinacolato)diboron (2.1 g), palladium acetate (0.05 g), XPhos (0.2 g), and potassium acetate (1.2 g) were added to 1,4-dioxane (50 mL). Under argon atmosphere, the obtained solution was stirred at 100 degrees C. for six hours. After being stirred, the reaction solution was purified by silica-gel column chromatography to obtain 1.1 g of a colorless solid (yield of 78%). As a result of FD-MS analysis, the colorless solid was the compound X-17 and m/e was equal to 344 while a molecular weight was 344.22.

A compound BH1-30 was synthesized through a synthesis pathway below.

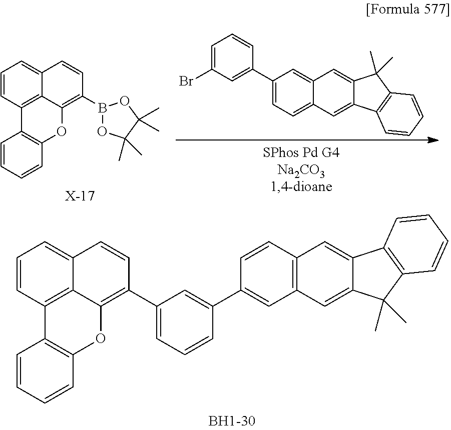 [SMILES: CC1(C)OB(c2ccc3cccc4c3c2Oc2ccccc2-4)OC1(C)C ; CC1(C)c2ccccc2-c2cc3ccc(-c4cccc(Br)c4)cc3cc21 ; "X-17" ; CC1(C)c2ccccc2-c2cc3ccc(-c4cccc(-c5ccc6cccc7c6c5Oc5ccccc5-7)c4)cc3cc21]

### Synthesis of Compound BH1-30

The compound X-17 (1.4 g), 8-(3-bromophenyl)-11,11-dimethyl-11H-benzo[b]fluorene (1.6 g), SPhos Pd G4 (0.06 g), and a sodium carbonate aqueous solution (2M, 6.1 mL) were added to 1,4-dioxane (40 mL). Under argon atmosphere, the obtained solution was stirred overnight at 90 degrees C. After the reaction solution was stirred, a solvent was distilled away from the reaction solution under reduced pressure. The obtained residue was recrystallized with toluene to obtain 1.9 g of a colorless solid (yield of 89%). As a result of FD-MS analysis, the colorless solid was the compound BH1-30 and m/e was equal to 537 while a molecular weight was 536.67.

### EXPLANATION OF CODE(S)

1 . . . organic EL device, 2 . . . substrate, 3 . . . anode, 4 . . . cathode, 51 . . . first emitting layer, 52 . . . second emitting layer, 6 . . . hole injecting layer, 7 . . . hole transporting layer, 8 . . . electron transporting layer, 9 . . . electron injecting layer

1: A compound comprising: at least one group represented by a formula (11) below; and a single benz[de]anthracene derivative skeleton represented by a formula (1000) below in a molecule, 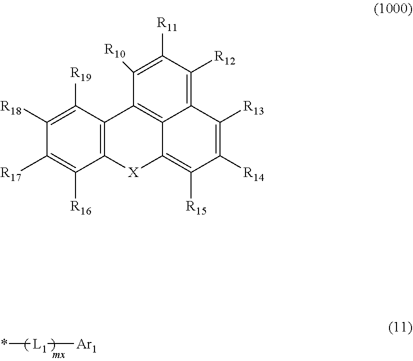
where: in the formula (1000):
X is an oxygen atom, a sulfur atom, C(R₂₀₀₁)(R₂₀₀₂), or Si(R₂₀₀₃)(R₂₀₀₄);
R₂₀₀₁ to R₂₀₀₄ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
at least one combination of adjacent two or more of R₁₀ to R₁₉ are mutually bonded to form a substituted or unsubstituted monocyclic ring, mutually bonded to form a substituted or unsubstituted fused ring, or not mutually bonded;
R₁₀ to R₁₉ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a nitro group, a substituted or unsubstituted aryl group having 6 to 50 ring carbon atoms, a substituted or unsubstituted heterocyclic group having 5 to 50 ring atoms, or a group represented by the formula (11);
at least one of R₁₀ to R₁₉ is a group represented by the formula (11);
when a plurality of groups represented by the formula (11) are present, the plurality of groups represented by the formula (11) are mutually the same or different;
L₁ is a substituted or unsubstituted arylene group having 6 to 15 ring carbon atoms, or a substituted or unsubstituted divalent heterocyclic group having 5 to 15 ring atoms;
mx is 1, 2, or 3;
when two or more L₁ are present, the two or more L₁ are mutually the same or different;
Ar₁ is a substituted or unsubstituted aryl group including four or more rings;
when two or more Ar₁ are present, the two or more Ar₁ are mutually the same or different;
* in the formula (11) represents a bonding position;
in a compound represented by the formula (1000), R₉₀₁, R₉₀₂, R₉₀₃, R₉₀₄, R₉₀₅, R₉₀₆, R₉₀₇, R₈₀₁, and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

2: The compound according to claim 1, wherein at least one combination of a combination of R₁₀ and R₁₁, a combination of R₁₁ and R₁₂, a combination of R₁₂ and R₁₃, a combination of R₁₃ and R₁₄, a combination of R₁₄ and R₁₅, a combination of R₁₆ and R₁₇, a combination of R₁₇ and R₁₈, a combination of R₁₈ and R₁₉, or a combination of R₁₉ and R₁₀ are mutually bonded to form a substituted or unsubstituted monocyclic ring, or mutually bonded to form a substituted or unsubstituted fused ring.

3: The compound according to claim 2, wherein one combination of the combination of R₁₃ and R₁₄, the combination of R₁₆ and R₁₇, and the combination of R₁₉ and R₁₀ are mutually bonded to form a substituted or unsubstituted benzene ring.

4: The compound according to claim 1, wherein the compound comprises: at least one group represented by the formula (11); and a single benzoxanthene ring represented by a formula (1) below in a molecule, 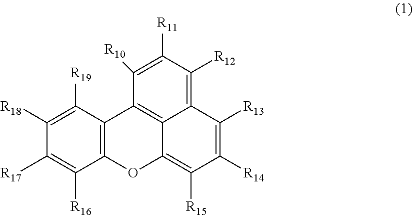
where: R₁₀ to R₁₉ each independently represent the same as R₁₀ to R₁₉ in the formula (1000);
none of combinations of adjacent two or more of R₁₀ to R₁₉ are mutually bonded; and
Ar₁, L₁, and mx respectively represent the same as Ar₁, L₁, and mx in the formula (11).

5: The compound according to claim 4, wherein the compound comprising at least one group represented by the formula (11) and a single benzoxanthene ring represented by the formula (1) in a molecule is represented by a formula (121) or (122), 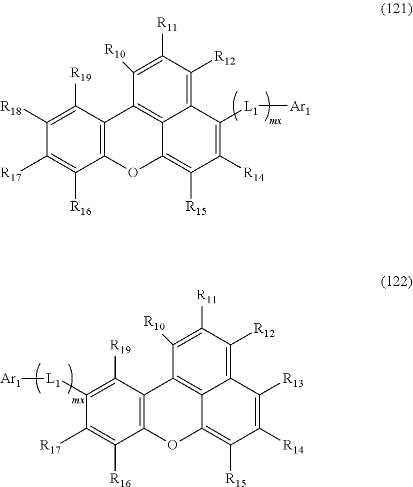
where: R₁₀ to R₁₉ each independently represent the same as R₁₀ to R₁₉ in the formula (1); and
Ar₁, L₁, and mx respectively represent the same as Ar₁, L₁, and mx in the formula (11).

6: The compound according to claim 1, wherein R₁₀ to R₁₉ not being a group represented by the formula (11) are each independently a hydrogen atom, a substituted or unsubstituted alkyl group having 1 to 50 carbon atoms, a substituted or unsubstituted cycloalkyl group having 3 to 50 ring carbon atoms, or a substituted or unsubstituted aryl group having 6 to 50 ring carbon atoms.

7: The compound according to claim 1, wherein Ar₁ is an aryl group in which at least four substituted or unsubstituted benzene rings are fused.

8: The compound according to claim 1, wherein the compound comprising at least one group represented by the formula (11) and a single benz[de]anthracene derivative skeleton represented by the formula (1000) in a molecule is represented by a formula (123), (124), (125), or (126) below, 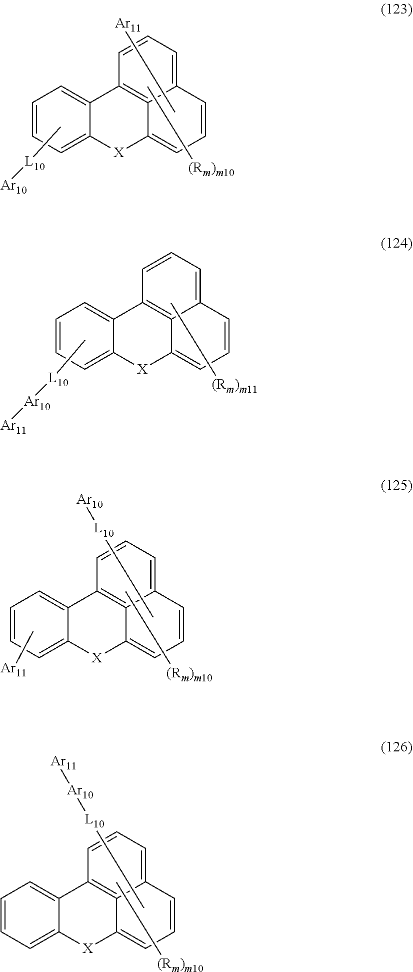
where: X is an oxygen atom, a sulfur atom, C(R₂₀₀₁)(R₂₀₀₂), or Si(R₂₀₀₃)(R₂₀₀₄);
R₂₀₀₁ to R₂₀₀₄ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
L₁₀ is a substituted or unsubstituted arylene group having 6 to 15 ring carbon atoms, or a substituted or unsubstituted divalent heterocyclic group having 5 to 15 ring atoms;
Ar₁₀ is an aryl group in which at least four substituted or unsubstituted benzene rings are fused;
Ar₁₁ is a substituted or unsubstituted aryl group having 6 to 14 ring carbon atoms;
Ar₁₁ is not a substituted or unsubstituted anthryl group;
m10 is 5;
m11 is 6;
Rm is each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a nitro group, a substituted or unsubstituted aryl group having 6 to 50 ring carbon atoms, or a substituted or unsubstituted heterocyclic group having 5 to 50 ring atoms;
a plurality of Rm are not mutually bonded;
R₉₀₁, R₉₀₂, R₉₀₃, R₉₀₄, R₉₀₅, R₉₀₆, R₉₀₇, R₈₀₁, and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

9: The compound according to claim 8, wherein a compound represented by the formula (123) is represented by a formula (1230) below, a compound represented by the formula (124) is represented by a formula (1240) below, a compound represented by the formula (125) is represented by a formula (1250) below, and a compound represented by the formula (126) is represented by a formula (1260) below, 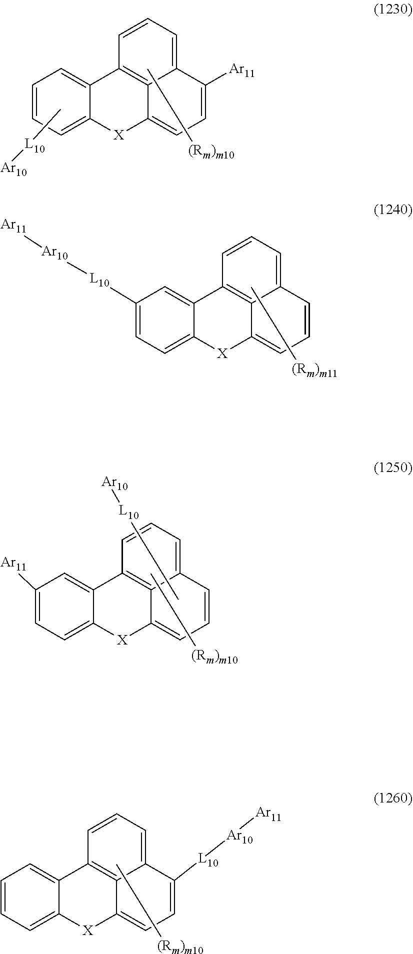
where: X, L₁₀, Ar₁₀, Ar₁₁, m10, m11, and Rm each independently represent the same as L₁₀, Ar₁₀, Ar₁₁, m10, m11, and Rm in the formulae (123), (124), (125), and (126).

10: The compound according to claim 8, wherein X is an oxygen atom.

11: A compound comprising: at least one group represented by a formula (110A) below; and a single benz[de]anthracene derivative skeleton represented by a formula (1000A) below in a molecule, 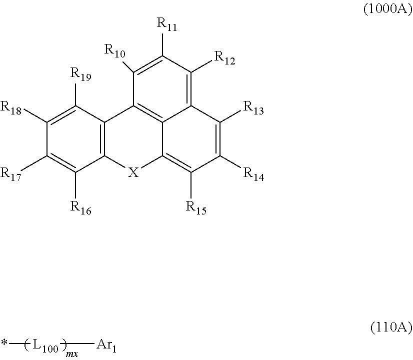
where in the formula (1000A):
X is an oxygen atom, a sulfur atom, C(R₂₀₀₁)(R₂₀₀₂), or Si(R₂₀₀₃)(R₂₀₀₄);
R₂₀₀₁ to R₂₀₀₄ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
at least one combination of adjacent two or more of R₁₀ to R₁₉ are mutually bonded to form a substituted or unsubstituted monocyclic ring, mutually bonded to form a substituted or unsubstituted fused ring, or not mutually bonded;
R₁₀ to R₁₉ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a nitro group, a substituted or unsubstituted aryl group having 6 to 50 ring carbon atoms, a substituted or unsubstituted heterocyclic group having 5 to 50 ring atoms, or a group represented by the formula (110A);
at least one of R₁₃ or R₁₈ is a group represented by the formula (110A);
when a plurality of groups represented by the formula (110A) are present, the plurality of groups represented by the formula (110A) are mutually the same or different;
L₁₀₀ is a single bond, a substituted or unsubstituted arylene group having 6 to 50 ring carbon atoms, or a substituted or unsubstituted divalent heterocyclic group having 5 to 50 ring atoms;
mx is 1, 2, or 3;
when two or more L₁₀₀ are present, the two or more L₁₀₀ are mutually the same or different;
Ar₁ is a substituted or unsubstituted aryl group including four or more rings;
when two or more Ar₁ are present, the two or more Ar₁ are mutually the same or different;
* in the formula (110A) represents a bonding position;
in a compound represented by the formula (1000A), R₉₀₁, R₉₀₂, R₉₀₃, R₉₀₄, R₉₀₅, R₉₀₆, R₉₀₇, R₈₀₁, and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

12: The compound according to claim 11, wherein at least one combination of a combination of R₁₀ and R₁₁, a combination of R₁ and R₁₂, a combination of R₁₂ and R₁₃, a combination of R₁₃ and R₁₄, a combination of R₁₄ and R₁₅, a combination of R₁₆ and R₁₇, a combination of R₁₇ and R₁₈, a combination of R₁₈ and R₁₉, or a combination of R₁₉ and R₁₀ are mutually bonded to form a substituted or unsubstituted monocyclic ring, or mutually bonded to form a substituted or unsubstituted fused ring.

13: The compound according to claim 12, wherein one combination of the combination of R₁₃ and R₁₄, the combination of R₁₆ and R₁₇, and the combination of R₁₉ and R₁₀ are mutually bonded to form a substituted or unsubstituted benzene ring.

14: The compound according to claim 11, wherein the compound comprises: at least one group represented by the formula (110A); and a single benzoxanthene ring represented by a formula (100A) below in a molecule, 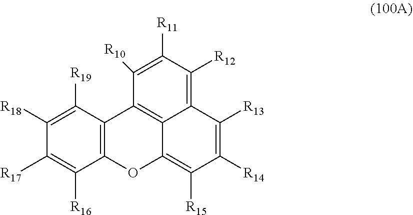
where: R₁₀ to R₁₉ each independently represent the same as R₁₀ to R₁₉ in the formula (1000A);
none of combinations of adjacent two or more of R₁₀ to R₁₉ are mutually bonded; and
Ar₁, L₁, and mx respectively represent the same as Ar₁, L₁, and mx in the formula (110A).

15: The compound according to claim 14, wherein the compound comprising at least one group represented by the formula (110A) and a single benzoxanthene ring represented by the formula (100A) below in a molecule is represented by a formula (121A) or (122A), 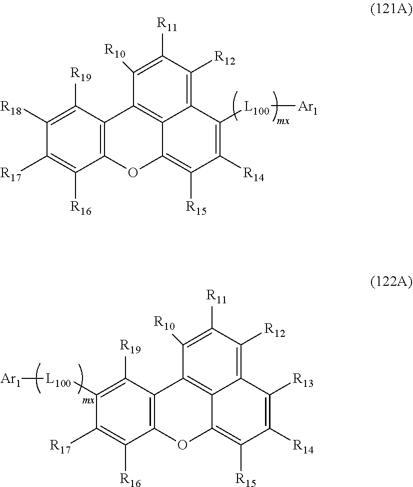
where: R₁₀ to R₁₉ each independently represent the same as R₁₀ to R₁₉ in the formula (100A), and
Ar₁, L₁₀₀, and mx respectively represent the same as Ar₁, L₁₀₀, and mx in the formula (110A).

16: The compound according to claim 11, wherein R₁₀ to R₁₉ not being a group represented by the formula (110A) are each independently a hydrogen atom, a substituted or unsubstituted alkyl group having 1 to 50 carbon atoms, a substituted or unsubstituted cycloalkyl group having 3 to 50 ring carbon atoms, or a substituted or unsubstituted aryl group having 6 to 50 ring carbon atoms.

17: The compound according to claim 1, wherein a group represented by -(L₁)ₘₓ- is a group represented by one of formulae (111) to (120) below, 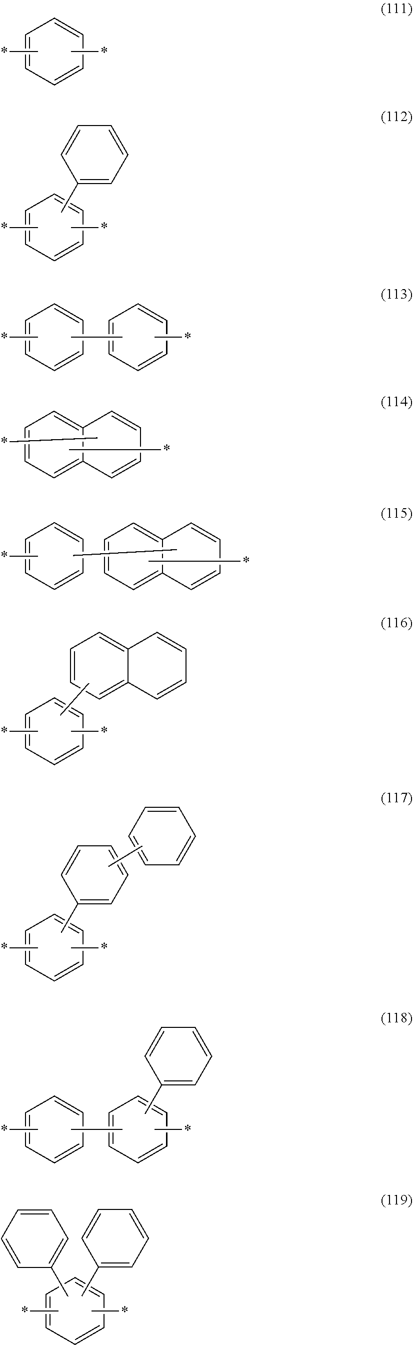 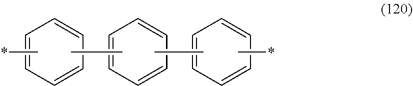
* in the formulae (111) to (120) represents a bonding position.

18: The compound according to claim 11, wherein Ar₁ is an aryl group in which at least four substituted or unsubstituted benzene rings are fused.

19: The compound according to claim 1, wherein Ar₁ is a group represented by a formula (1100), (1200), (1300), (1400), (1500), (1600), (1700), or (1800) below, 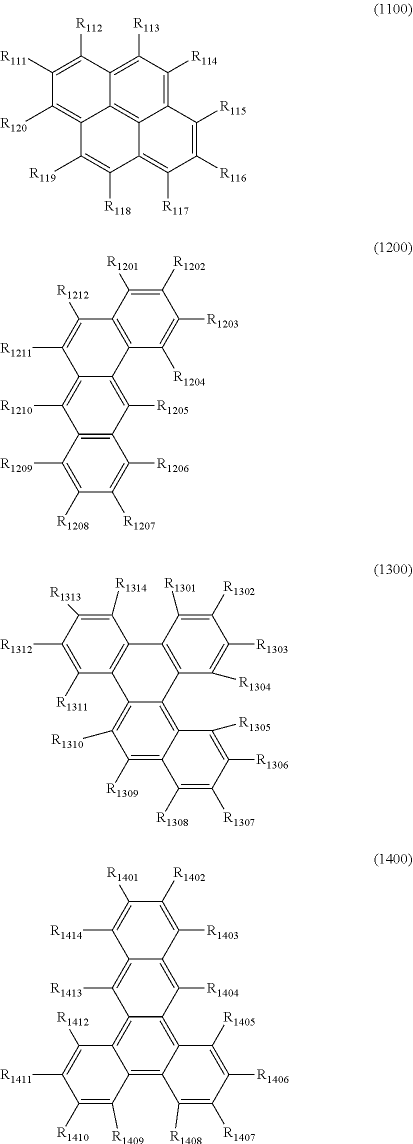 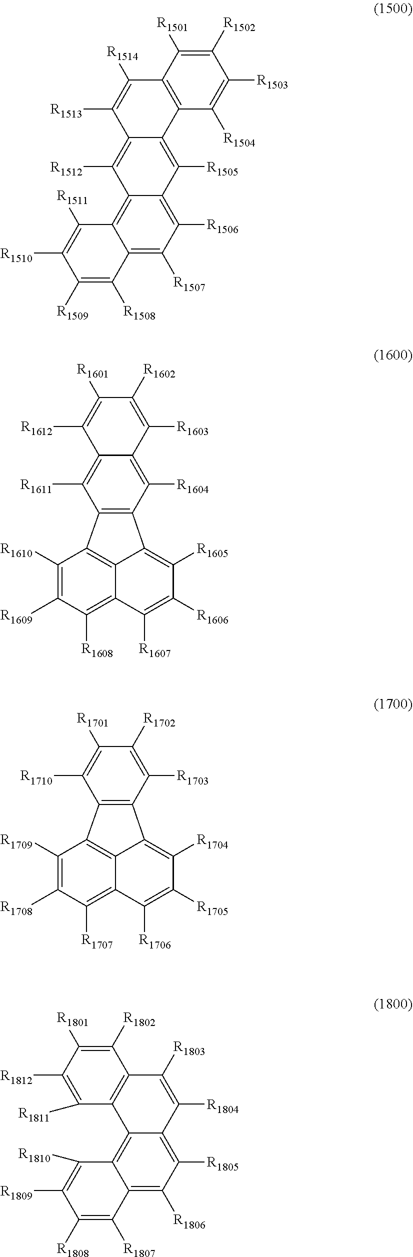
where: in the formula (1100), one of R₁₁₁ to R₁₂₀ is a bond;
in the formula (1200), one of R₁₂₀₁ to R₁₂₁₂ is a bond;
in the formula (1300), one of R₁₃₀₁ to R₁₃₁₄ is a bond;
in the formula (1400), one of R₁₄₀₁ to R₁₄₁₄ is a bond;
in the formula (1500), one of R₁₅₀₁ to R₁₅₁₄ is a bond;
in the formula (1600), one of R₁₆₀₁ to R₁₆₁₂ is a bond;
in the formula (1700), one of R₁₇₀₁ to R₁₇₁₀ is a bond;
in the formula (1800), one of R₁₈₀₁ to R₁₈₁₂ is a bond;
R₁₁ to R₁₂₀ not being a bond, R₁₂₀₁ to R₁₂₁₂ not being a bond, R₁₃₀₁ to R₁₃₁₄ not being a bond, R₁₄₀₁ to R₁₄₁₄ not being a bond, R₁₅₀₁ to R₁₅₁₄ not being a bond, R₁₆₀₁ to R₁₆₁₂ not being a bond, R₁₇₀₁ to R₁₇₁₀ not being a bond, and R₁₈₈₀ to R₁₈₁₂ not being a bond are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a nitro group, a substituted or unsubstituted aryl group having 6 to 50 ring carbon atoms, or a substituted or unsubstituted heterocyclic group having 5 to 50 ring atoms.

20: The compound according to claim 19, wherein R₁₁₁ to R₁₂₀ not being a bond, R₁₂₀₁ to R₁₂₁₂ not being a bond, R₁₃₀₁ to R₁₃₁₄ not being a bond, R₁₄₀₁ to R₁₄₁₄ not being a bond, R₁₅₀₁ to R₁₅₁₄ not being a bond, R₁₆₀₁ to R₁₆₁₂ not being a bond, R₁₇₀₁ to R₁₇₁₀ not being a bond, and R₁₈₀₁ to R₁₈₁₂ not being a bond are each independently a hydrogen atom, a substituted or unsubstituted alkyl group having 1 to 50 carbon atoms, a substituted or unsubstituted cycloalkyl group having 3 to 50 ring carbon atoms, or a substituted or unsubstituted aryl group having 6 to 50 ring carbon atoms.

21: The compound according to claim 1, wherein all of the “substituted or unsubstituted” groups are “unsubstituted” groups.

22: An organic electroluminescence device comprising:
an anode;
a cathode; and
at least one organic layer disposed between the anode and the cathode, wherein
the at least one organic layer comprises an emitting layer, and
at least one of the at least one organic layer comprises a compound according to claim 1.

23: The organic electroluminescence device according to claim 22, wherein the emitting layer comprises the compound.

24: An organic electroluminescence device comprising:
an anode;
a cathode; and
at least one emitting layer disposed between the anode and the cathode, wherein
the at least one emitting layer comprises a first emitting layer and a second emitting layer,
the first emitting layer comprises a first compound, and
the first compound is a compound represented by a formula (1000B) below and comprising at least one group represented by a formula (110) below, 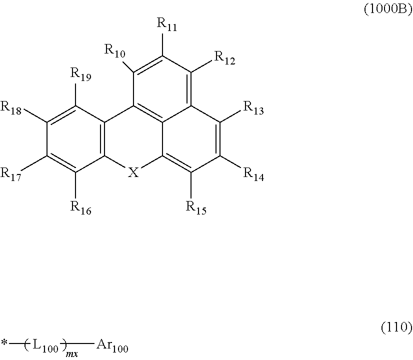
where: in the formula (1000B):
X is an oxygen atom, a sulfur atom, C(R₂₀₀₁)(R₂₀₀₂), or Si(R₂₀₀₃)(R₂₀₀₄);
R₂₀₀₁ to R₂₀₀₄ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R_{9O7}), a halogen atom, a cyano group, a nitro group, a substituted or unsubstituted aryl group having 6 to 50 ring carbon atoms, or a substituted or unsubstituted heterocyclic group having 5 to 50 ring atoms;
at least one combination of adjacent two or more of R₁₀ to R₁₉ are mutually bonded to form a substituted or unsubstituted monocyclic ring, mutually bonded to form a substituted or unsubstituted fused ring, or not mutually bonded;
R₁₀ to R₁₉ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a nitro group, a substituted or unsubstituted aryl group having 6 to 50 ring carbon atoms, a substituted or unsubstituted heterocyclic group having 5 to 50 ring atoms, or a group represented by the formula (110);
at least one of R₁₀ to R₁₉ is a group represented by the formula (110);
when a plurality of groups represented by the formula (110) are present, the plurality of groups represented by the formula (110) are mutually the same or different;
L₁₀₀ is a single bond, a substituted or unsubstituted arylene group having 6 to 50 ring carbon atoms, or a substituted or unsubstituted divalent heterocyclic group having 5 to 50 ring atoms;
mx is 1, 2, or 3;
when two or more L₁₀₀ are present, the two or more L₁₀₀ are mutually the same or different;
Ar₁₀₀ is a substituted or unsubstituted aryl group including three or more rings or a substituted or unsubstituted heterocyclic group including two or more aromatic rings and one or more heterocyclic rings;
Ar₁₀₀ does not include an anthracene ring;
when two or more Ar₁₀₀ are present, the two or more Ar₁₀₀ are mutually the same or different; and
* in the formula (110) represents a bonding position;
in the first compound represented by the formula (1000B), R₉₀₁, R₉₀₂, R₉₀₃, R₉₀₄, R₉₀₅, R₉₀₆, R₉₀₇, R₈₀₁, and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

25-43. (canceled)

44: The organic electroluminescence device according to claim 24, wherein
the second emitting layer further comprises a fourth compound that fluoresces, and
the fourth compound is a compound that emits light having a main peak wavelength in a range from 430 nm to 480 nm.

45: An electronic device comprising the organic electroluminescence device according to claim 23.
